(19) United States
(12) Patent Application Publication
(10) Pub. No.: US 2025/0276090 A1
(43) Pub. Date: Sep. 4, 2025

(54) Methods and Compositions for Genetically Modifying a Cell

(71) Applicant: Intellia Therapeutics, Inc., Cambridge, MA (US)

(72) Inventors: Birgit Schultes, Arlington, MA (US); Aaron Prodeus, Cambridge, MA (US); Özgün Kiliç, Cambridge, MA (US); Ruan Oliveira, Boston, MA (US); Christian Dombrowski, Auburndale, MA (US)

(73) Assignee: Intellia Therapeutics, Inc., Cambridge, MA (US)

(21) Appl. No.: 18/980,643

(22) Filed: Dec. 13, 2024

Related U.S. Application Data
(63) Continuation of application No. PCT/US23/68507, filed on Jun. 15, 2023.
(60) Provisional application No. 63/353,008, filed on Jun. 16, 2022.

(51) Int. Cl. A61K 48/00; C12N 9/12; C12N 9/22; C12N 15/11; C12N 15/88

(52) U.S. Cl. CPC A61K 48/005; C12N 9/1258; C12N 9/226; C12N 15/111; C12N 15/88; C07K 2319/80; C12N 2310/20; C12Y 207/07007

(57) ABSTRACT

Methods and compositions for genetically modifying a cell are provided.

### CROSS-REFERENCE TO RELATED APPLICATION

This application is a continuation of International Application No. PCT/US2023/068507, filed Jun. 15, 2023, which claims the benefit under 35 USC 119(e) of U.S. Provisional Application No. 63/353,008, filed Jun. 16, 2022, the content of each of which is herein incorporated by reference in its entirety.

### SEQUENCE LISTING

This application contains a sequence listing, which has been submitted electronically in XML file format and is hereby incorporated by reference in its entirety. Said XML file, created on Jun. 13, 2023, is named “01155-0060-00PCT_SL.xml” and is 2,718,632 bytes in size.

### INTRODUCTION AND SUMMARY

The ability to introduce multiple genetic edits into a cell is of interest for gene editing and clinical therapeutic applications. For example, adoptive cell therapy approaches using genetically modified immune cells have become an attractive modality to treat a variety of conditions and diseases, including cancers, to reconstitute cell lineages and immune system defense. However, the clinical application of cell product therapies has been challenging in part due to the complex genetic engineering requirements. The ability to engineer multiple attributes into a single cell depends on the ability to efficiently perform edits in multiple targeted genes, including knockouts and in locus insertions, while retaining viability and desired cell phenotypes.

CRISPR/Cas9 genome editing has been demonstrated to be highly efficient; however, simultaneous edits in different loci have been reported to result in poorer cell survival, increased translocations, which potentially impair the quality and safety of the cell product, and decreased gene editing efficiencies as the number of edits increase. Existing cell engineering technologies present limitations in providing the necessary cell quality and yield using a sequential editing process due to the cumulative toxicity to the cell.

Thus, there is a need for safer, more efficient processes for delivering multiple genome editing tools to a cell and for performing multiplexed gene editing, for example with fewer steps or within a shorter time period.

The methods provided herein comprise using at least two genome editing tools for multiplex genome editing applications, providing substantial advantages over traditional methods.

In some embodiments, the methods provided herein produce cells with greater survival and expansion, while maintaining high editing rates, thereby shortening the time required for manufacturing and increasing yield.

### BRIEF DESCRIPTION OF THE DRAWINGS

FIGS. 1A-1C show percent T cells lacking HLA-A surface expression following simultaneous insertion and base editing in 3 donors.

FIGS. 2A-2C show percent T cells lacking CD3 surface expression following simultaneous insertion and base editing in 3 donors.

FIGS. 3A-3C show percent T cells expressing transgenic T cell receptor following simultaneous insertion and base editing in 3 donors.

FIGS. 4A-4H show percent editing in T cells following simultaneous insertion and base editing in 3 donors.

FIG. 5A shows percent T cells showing full editing markers following simultaneous insertion and base editing using lipid nanoparticles in 4 donors.

FIG. 5B shows percent T cells lacking CD3 surface expression following simultaneous insertion and base editing using lipid nanoparticles in 4 donors.

FIG. 5C shows percent T cells lacking HLA-A2 surface expression, HLA-A3 surface expression, or both following simultaneous insertion and base editing using lipid nanoparticles in 4 donors.

FIG. 5D shows percent T cells lacking HLA-DP, DQ, DR surface expression following simultaneous insertion and base editing using lipid nanoparticles in 4 donors.

FIG. 5E shows percent T cells positive for surface expression the transgenic TCR following simultaneous insertion and base editing using lipid nanoparticles in 4 donors.

FIG. 6A shows percent editing at the albumin locus and relative luminescence in primary mouse hepatocytes.

FIG. 6B shows mean percent editing at the TTR locus in primary mouse hepatocytes.

FIG. 7A shows percent editing at the TTR locus in mouse liver.

FIG. 7B shows percent editing at the albumin locus in mouse liver.

FIG. 7C shows serum A1AT levels.

FIG. 8A shows percent GFP positive Donor 1 T cells following insertion at AAVS1.

FIG. 8B shows percent GFP positive Donor 2 T cells following insertion at AAVS1.

FIG. 9 shows the fold increase in cell population after the indicated days in expansion media.

FIGS. 10A-10B show the mean percent of full edited T cells with the CD4+ and CD8+ subpopulations, respectively

FIGS. 11A-11C show mean percent editing for TRAC, TRBC1, TRBC2 and CIITA loci after base editing.

FIG. 12A shows mean percent of CD8+ T cells scored as CD3− or Vb8+ by flow cytometry. FIG. 12B shows the mean percent of CD8+ T cells scored as negative for HLA-DP, DQ, DR, HLA-A2 OR HLA-A3 surface markers by flow cytometry.

FIG. 13A shows the mean percent of CD8+ engineered T cells displaying central memory stem cell phenotype. FIG. 13B shows the mean percent of CD8+ engineered T cells displaying markers for central memory cell phenotype. FIG. 13C shows the mean percent of CD8+ engineered T cells displaying markers for effector memory cell phenotype.

FIG. 14 shows mean percent target cell killing by engineered T cells.

FIG. 15 shows mean percent editing after treatment with 1.0 ug/ml or 0.5 ug·ml base editor mRNA.

FIG. 16 shows mean percent of T cells negative for indicated surface protein expression.

### BRIEF DESCRIPTION OF DISCLOSED SEQUENCES

| SEQ ID | |
|---|---|
| NO | Description |
| 1 | mRNA encoding SpyCas9 BC22n |
| 2 | Open reading frame for Sp BC22n |
| 3 | Amino acid sequence for Sp BC22n |
| 4 | mRNA encoding Sp BC22n with Hibit tag |
| 5 | Open reading frame for Sp BC22n with Hibit tag |
| 6 | Amino acid sequence for Sp BC22n with Hibit tag |
| 7 | mRNA encoding BE3 |
| 8 | Open reading frame for BE3 |
| 9 | Amino acid sequence for BE3 |
| 10 | mRNA encoding BE3 |
| 11 | Open reading frame for BE3 |
| 12 | Amino acid sequence for BE3 |
| 13 | mRNA encoding UGI |
| 14 | Open reading frame for UGI |
| 15 | Amino acid sequence for UGI |
| 16 | mRNA encoding Sp BC22 with 2x UGI |
| 17 | Open reading frame for Sp BC22 with 2x UGI |
| 18 | Amino acid sequence for Sp BC22 with 2x UGI |
| 19 | mRNA encoding BE4MAX protein |
| 20 | Open reading frame for BE4MAX protein |
| 21 | Amino acid sequence for BE4MAX protein |
| 22 | Amino acid sequence of H. sapiens APOBEC3A deaminase (A3A), see BC22 |
| 23 | |
| 24 | exemplary UGI |
| 25 | exemplary XTEN |
| 26 | exemplary XTEN |
| 27 | exemplary XTEN |
| 28 | amino acid sequence for exemplary linker |
| 29 | amino acid sequence for exemplary linker |
| 30 | amino acid sequence for exemplary linker |
| 31 | amino acid sequence for exemplary linker |
| 32 | amino acid sequence for exemplary linker |
| 33 | amino acid sequence for exemplary linker |
| 34 | amino acid sequence for exemplary linker |
| 35 | amino acid sequence for exemplary linker |
| 36 | amino acid sequence for exemplary linker |
| 37 | amino acid sequence for exemplary linker |
| 38 | amino acid sequence for exemplary linker |
| 39 | amino acid sequence for exemplary linker SGGS |
| 40 | amino acid acid sequence for SV40 NLS |
| 41 | Amino acid sequence of Sp Cas9 nickase (D10A) with 1x NLS as the C- |
| | terminal 7 amino acids |
| 42 | Sp Cas9 nickase (D10A) mRNA coding sequence using minimal uridine |
| | codons as listed in Table 3 (no start or stop codons; suitable for inclusion in |
| | fusion protein coding sequence) |
| 43 | Amino acid sequence of Sp Cas9 nickase (without NLS) |
| 44 | Cas9 nickase coding sequence encoding SEQ ID NO: 43 using minimal |
| | uridine codons as listed in Table 3 (no start or stop codons; suitable for |
| | inclusion in fusion protein coding sequence) |
| 45 | Amino acid sequence of Sp Cas9 nickase with two nuclear localization signals |
| | as the C-terminal amino acids |
| 46 | Sp Cas9 nickase coding sequence encoding SEQ ID NO: 45 using minimal |
| | uridine codons as listed in Table 3 (no start or stop codons; suitable for |
| | inclusion in fusion protein coding sequence) |
| 47 | Sp Cas9 nickase ORF using low A codons of Table 4, with start and stop |
| | codons |
| 48 | Sp Cas9 nickase ORF using low A codons of Table 4, with start and stop |
| | codons and no NLS |
| 49 | Sp Cas9 nickase ORF using low A codons of Table 4, with two C-terminal |
| | NLS sequences and start and stop codons |
| 50 | Sp Cas9 nickase ORF using low A/U codons of Table 4, with start and stop |
| | codons |
| 51 | Sp Cas9 nickase ORF using low A/U codons of Table 4, with two C-terminal |
| | NLS sequences and start and stop codons |
| 52 | Sp Cas9 nickase ORF using low A/U codons of Table 4, with start and stop |
| | codons and no NLS |
| 53 | Sp Cas9 nickase ORF using low A codons of Table 4 (no start or stop codons; |
| | suitable for inclusion in fusion protein coding sequence) |
| 54 | Sp Cas9 nickase ORF using low A codons of Table 4 (no NLS and no start or |
| | stop codons; suitable for inclusion in fusion protein coding sequence) |
| 55 | Sp Cas9 nickase ORF using low A codons of Table 4, with two C-terminal |
| | NLS sequences (no start or stop codons; suitable for inclusion in fusion |
| | protein coding sequence) |
| 56 | Sp Cas9 nickase ORF using low A/U codons of Table 4 (no start or stop |
| | codons; suitable for inclusion in fusion protein coding sequence) |
| 57 | Sp Cas9 nickase ORF using low A/U codons of Table 4, with two C-terminal |
| | NLS sequences (no start or stop codons; suitable for inclusion in fusion |
| | protein coding sequence) |
| 58 | Sp Cas9 nickase ORF using low A/U codons of Table 4 (no NLS and no start |
| | or stop codons; suitable for inclusion in fusion protein coding sequence) |
| 59 | Exemplary NLS 1 |
| 60 | Exemplary NLS 2 |
| 61 | Exemplary NLS 3 |
| 62 | Exemplary NLS 4 |
| 63 | Exemplary NLS 5 |
| 64 | Exemplary NLS 6 |
| 65 | Exemplary NLS 7 |
| 66 | Exemplary NLS 8 |
| 67 | Exemplary NLS 9 |
| 68 | Exemplary NLS 10 |
| 69 | Exemplary NLS 11 |
| 70 | Alternate SV40 NLS |
| 71 | Nucleoplasmin NLS |
| 72 | amino acid sequence for exemplary linker |
| 73 | amino acid sequence for exemplary linker |
| 74 | amino acid sequence for exemplary linker |
| 75 | amino acid sequence for exemplary linker |
| 76 | amino acid sequence for exemplary linker |
| 77 | amino acid sequence for exemplary linker |
| 78 | amino acid sequence for exemplary linker |
| 79 | amino acid sequence for exemplary linker |
| 80 | amino acid sequence for exemplary linker |
| 81 | amino acid sequence for exemplary linker |
| 82 | amino acid sequence for exemplary linker |
| 83 | amino acid sequence for exemplary linker |
| 84 | amino acid sequence for exemplary linker |
| 85 | amino acid sequence for exemplary linker |
| 86 | amino acid sequence for exemplary linker |
| 87 | amino acid sequence for exemplary linker |
| 88 | amino acid sequence for exemplary linker |
| 89 | amino acid sequence for exemplary linker |
| 90 | amino acid sequence for exemplary linker |
| 91 | amino acid sequence for exemplary linker |
| 92 | amino acid sequence for exemplary linker |
| 93 | amino acid sequence for exemplary linker |
| 94 | amino acid sequence for exemplary linker |
| 95 | amino acid sequence for exemplary linker |
| 96 | amino acid sequence for exemplary linker |
| 97 | amino acid sequence for exemplary linker |
| 98 | amino acid sequence for exemplary linker |
| 99 | amino acid sequence for exemplary linker |
| 100 | amino acid sequence for exemplary linker |
| 101 | amino acid sequence for exemplary linker |
| 102 | amino acid sequence for exemplary linker |
| 103 | amino acid sequence for exemplary linker |
| 104 | amino acid sequence for exemplary linker |
| 105 | amino acid sequence for exemplary linker |
| 106 | amino acid sequence for exemplary linker |
| 107 | amino acid sequence for exemplary linker |
| 108 | amino acid sequence for exemplary linker |
| 109 | amino acid sequence for exemplary linker |
| 110 | amino acid sequence for exemplary linker |
| 111 | amino acid sequence for exemplary linker |
| 112 | amino acid sequence for exemplary linker |
| 113 | amino acid sequence for exemplary linker |
| 114 | amino acid sequence for exemplary linker |
| 115 | amino acid sequence for exemplary linker |
| 116 | amino acid sequence for exemplary linker |
| 117 | amino acid sequence for exemplary linker |
| 118 | amino acid sequence for exemplary linker |
| 119 | amino acid sequence for exemplary linker |
| 120 | amino acid sequence for exemplary linker |
| 121 | amino acid sequence for exemplary linker |
| 122 | amino acid sequence for exemplary linker |
| 123 | amino acid sequence for exemplary linker |
| 124 | amino acid sequence for exemplary linker |
| 125 | amino acid sequence for exemplary linker |
| 126 | amino acid sequence for exemplary linker |
| 127 | amino acid sequence for exemplary linker |
| 128 | amino acid sequence for exemplary linker |
| 129 | amino acid sequence for exemplary linker |
| 130 | amino acid sequence for exemplary linker |
| 131 | amino acid sequence for exemplary linker |
| 132 | amino acid sequence for exemplary linker |
| 133 | amino acid sequence for exemplary linker |
| 134 | Exemplary mRNA encoding APOBEC3A-Nme2D16A |
| 135 | Exemplary open reading frame for APOBEC3A-Nme2D16A |
| 136 | Exemplary amino acid sequence for APOBEC3A-Nme2D16A |
| 137 | Exemplary mRNA encoding APOBEC3A-Nme2D16A |
| 138 | Exemplary open reading frame for APOBEC3A-Nme2D16A |
| 139 | Exemplary amino acid sequence for APOBEC3A-Nme2D16A |
| 140 | Exemplary mRNA encoding APOBEC3A-Nme2D16A |
| 141 | Exemplary open reading frame for APOBEC3A-Nme2D16A |
| 142 | Exemplary amino acid sequence for APOBEC3A-Nme2D16A |
| 143 | EXEMPLARY MRNA ENCODING APOBEC3A-NME2D16A |
| 144 | Exemplary open reading frame for APOBEC3A-Nme2D16A |
| 145 | Exemplary amino acid sequence for APOBEC3A-Nme2D16A |
| 146 | Exemplary amino acid sequence for NLS-NLS-APOBEC3A-L070- |
| | Nme2D16A |
| 147 | mRNA encoding BC22-Nme2D16A (Nme2 BC22n) |
| 148 | Amino acid sequence for base editor with linker L070 |
| 149 | amino acid sequence for D16A Nme2Cas9 nickase |
| 150 | coding sequence for D16A Nme2Cas9 nickase |
| 151 | coding sequence for D16A Nme2Cas9 nickase |
| 152 | coding sequence for D16A Nme2Cas9 nickase |
| 153 | open reading frame for D16A Nme2Cas9 nickase |
| 154 | open reading frame for D16A Nme2Cas9 nickase |
| 155 | open reading frame for D16A Nme2Cas9 nickase |
| 156 | Cas9 amino acid sequence |
| 157 | Amino acid sequence for Nme2Cas9 encoded by mRNA C |
| 158 | Amino acid sequence for Nme2Cas9 encoded by mRNA H |
| 159 | Amino acid sequence for Nme2Cas9 encoded by mRNA I |
| 160 | Amino acid sequence for Nme2Cas9 encoded by mRNA J |
| 161 | Amino acid sequence for Nme2Cas9 encoded by mRNA K |
| 162 | Amino acid sequence for Nme2Cas9 encoded by mRNA L |
| 163 | Amino acid sequence for Nme2Cas9 with HiBiT tag encoded by mRNA M |
| 164 | Amino acid sequence for Nme2Cas9 encoded by mRNA N |
| 165 | Amino acid sequence for Nme2Cas9 encoded by mRNA O |
| 166 | Amino acid sequence for Nme2Cas9 with HiBiT tag encoded by mRNA P |
| 167 | Amino acid sequence for Nme2Cas9 encoded by mRNA Q |
| 168 | mRNA C encoding Nme2Cas9 |
| 169 | mRNA H encoding Nme2Cas9 |
| 170 | mRNA I encoding Nme2Cas9 |
| 171 | mRNA J encoding Nme2Cas9 |
| 172 | mRNA K encoding Nme2Cas9 |
| 173 | mRNA L encoding Nme2Cas9 |
| 174 | mRNA M encoding Nme2Cas9 with HiBiT tag |
| 175 | mRNA N encoding Nme2Cas9 |
| 176 | mRNA O encoding Nme2Cas9 |
| 177 | mRNA P encoding Nme2Cas9 with HiBiT tag |
| 178 | mRNA Q encoding Nme2Cas9 |
| 179 | mRNA S encoding Nme2Cas9 base editor |
| 180 | Open reading frame for Nme2Cas9 encoded by mRNA C |
| 181 | Open reading frame for Nme2Cas9 encoded by mRNA H |
| 182 | Open reading frame for Nme2Cas9 encoded by mRNA I |
| 183 | Open reading frame for Nme2Cas9 encoded by mRNA J |
| 184 | Open reading frame for Nme2Cas9 encoded by mRNA K |
| 185 | Open reading frame for Nme2Cas9 encoded by mRNA L |
| 186 | Open reading frame for Nme2Cas9 with HiBiT tag encoded by mRNA M |
| 187 | Open reading frame for Nme2Cas9 encoded by mRNA N |
| 188 | Open reading frame for Nme2Cas9 encoded by mRNA O |
| 189 | Open reading frame for Nme2Cas9 with HiBiT tag encoded by mRNA P |
| 190 | Open reading frame for Nme2Cas9 encoded by mRNA Q |
| 191 | Exemplary amino acid sequence of Nme1Cas9 cleavase |
| 192 | Exemplary coding sequence encoding Nme1Cas9 cleavase |
| 193 | Exemplary coding sequence encoding Nme1Cas9 cleavase |
| 194 | Exemplary coding sequence encoding Nme1Cas9 cleavase |
| 195 | Exemplary open reading frame for Nme1Cas9 cleavase |
| 196 | Exemplary open reading frame for Nme1Cas9 cleavase |
| 197 | Exemplary open reading frame for Nme1Cas9 cleavase |
| 198 | Exemplary amino acid sequence of Nme1Cas9 HNH nickase |
| 199 | Exemplary coding sequence encoding Nme1Cas9 HNH nickase |
| 200 | Exemplary coding sequence encoding Nme1Cas9 HNH nickase |
| 201 | Exemplary coding sequence encoding Nme1Cas9 HNH nickase |
| 202 | Exemplary open reading frame for Nme1Cas9 HNH nickase |
| 203 | Exemplary open reading frame for Nme1Cas9 HNH nickase |
| 204 | Exemplary open reading frame for Nme1Cas9 HNH nickase |
| 205 | Exemplary amino acid sequence of Nme2Cas9 cleavase |
| 206 | Exemplary coding sequence encoding Nme2Cas9 cleavase |
| 207 | Exemplary coding sequence encoding Nme2Cas9 cleavase |
| 208 | Exemplary coding sequence encoding Nme2Cas9 cleavase |
| 209 | Exemplary open reading frame for Nme2Cas9 cleavase |
| 210 | Exemplary open reading frame for Nme2Cas9 cleavase |
| 211 | Exemplary open reading frame for Nme2Cas9 cleavase |
| 212 | Exemplary amino acid sequence of Nme3Cas9 cleavase |
| 213 | Exemplary coding sequence encoding Nme3Cas9 cleavase |
| 214 | Exemplary coding sequence encoding Nme3Cas9 cleavase |
| 215 | Exemplary coding sequence encoding Nme3Cas9 cleavase |
| 216 | Exemplary open reading frame for Nme3Cas9 cleavase |
| 217 | Exemplary open reading frame for Nme3Cas9 cleavase |
| 218 | Exemplary open reading frame for Nme3Cas9 cleavase |
| 219 | Exemplary amino acid sequence of Nme3Cas9 HNH nickase |
| 220 | Exemplary coding sequence encoding Nme3Cas9 HNH nickase |
| 221 | Exemplary coding sequence encoding Nme3Cas9 HNH nickase |
| 222 | Exemplary coding sequence encoding Nme3Cas9 HNH nickase |
| 223 | Exemplary open reading frame for Nme3Cas9 HNH nickase |
| 224 | Exemplary open reading frame for Nme3Cas9 HNH nickase |
| 225 | Exemplary open reading frame for Nme3Cas9 HNH nickase |
| 226 | Exemplary SpyCas9 sgRNA-1 |
| 227 | Exemplary nucleotide sequence following 3′ end of guide sequence |
| 228 | Exemplary modified SpyCas9 motif |
| 229 | Exemplary modified SpyCas9 conserved portion motif |
| 230 | Exemplary modified SpyCas9 conserved portion motif |
| 231 | Exemplary modified SpyCas9 conserved portion motif |
| 232 | Exemplary modified SpyCas9 conserved portion motif |
| 233 | Exemplary modified SpyCas9 conserved portion motif |
| 234 | Exemplary modified SpyCas9 conserved portion motif |
| 235 | Exemplary modified SpyCas9 conserved portion motif |
| 236 | Exemplary modified SpyCas9 conserved portion motif |
| 237 | Exemplary modified SpyCas9 conserved portion motif |
| 238 | Exemplary modified SpyCas9 conserved portion motif |
| 239 | Exemplary modified SpyCas9 conserved portion motif |
| 240 | Exemplary modified SpyCas9 conserved portion motif |
| 241 | Exemplary modified SpyCas9 conserved portion motif |
| 242 | Exemplary modified SpyCas9 conserved portion motif |
| 243 | Exemplary unmodified conserved portion nucleotide sequence |
| 244 | Exemplary unmodified conserved portion nucleotide sequence |
| 245 | Exemplary unmodified conserved portion nucleotide sequence |
| 246 | Exemplary modified conserved portion motif |
| 247 | Exemplary modified conserved portion motif |
| 248 | Exemplary modified conserved portion motif |
| 249 | Exemplary modified conserved portion motif |
| 250 | Exemplary modified conserved portion motif |
| 251 | G000562 |
| 252 | G013515 |
| 253 | G013519 |
| 254 | G013520 |
| 255 | G013523 |
| 256 | G013533 |
| 257 | G013543 |
| 258 | G013559 |
| 259 | G013562 |
| 260 | G013563 |
| 261 | G013564 |
| 262 | G013565 |
| 263 | G013582 |
| 264 | G013584 |
| 265 | G000562 (exemplary full sequence) |
| 266 | G013515 (exemplary full sequence) |
| 267 | G013519 (exemplary full sequence) |
| 268 | G013520 (exemplary full sequence) |
| 269 | G013523 (exemplary full sequence) |
| 270 | G013533 (exemplary full sequence) |
| 271 | G013543 (exemplary full sequence) |
| 272 | G013559 (exemplary full sequence) |
| 273 | G013562 (exemplary full sequence) |
| 274 | G013563 (exemplary full sequence) |
| 275 | G013564 (exemplary full sequence) |
| 276 | G013565 (exemplary full sequence) |
| 277 | G013582 (exemplary full sequence) |
| 278 | G013584 (exemplary full sequence) |
| 279 | G000562 (exemplary mod sequence) |
| 280 | G013515 (exemplary mod sequence) |
| 281 | G013519 (exemplary mod sequence) |
| 282 | G013520 (exemplary mod sequence) |
| 283 | G013523 (exemplary mod sequence) |
| 284 | G013533 (exemplary mod sequence) |
| 285 | G013543 (exemplary mod sequence) |
| 286 | G013559 (exemplary mod sequence) |
| 287 | G013562 (exemplary mod sequence) |
| 288 | G013563 (exemplary mod sequence) |
| 289 | G013564 (exemplary mod sequence) |
| 290 | G013565 (exemplary mod sequence) |
| 291 | G013582 (exemplary mod sequence) |
| 292 | G013584 (exemplary mod sequence) |
| 293 | Open reading frame for Cas9 |
| 294 | Amino acid sequence for Cas9 |
| 295 | Open reading frame for Cas9 |
| 296 | Amino acid sequence for Cas9-NLS |
| 297 | TCR insertion construct with homology arms flanking TRAC G013006 cut |
| | site - ITR included |
| 298 | bidirectional SERPINA insertion construct |
| 299 | Template A eGFP insertion construct with homology arms to mouse AAVS1 |
| 300 | Template B eGFP insertion construct with homology arms to mouse AAVS1 |
| 301 | Template C eGFP insertion construct with homology arms to mouse AAVS1 |
| 302 | Template D eGFP insertion construct with homology arms to mouse AAVS1 |
| 303 | Template OG eGFP insertion construct with homology arms to mouse |
| | AAVS1 |
| 304 | Bidirectional NanoLuc insertion Construct |
| 305 | Open reading frame for Nme2 Cas9 |
| 306 | Open reading frame for Sp Base Editor BC22n |
| 307 | Open reading frame for Sp Cas9 |
| 308 | Open reading frame for Nme2 Base Editor BC22n |
| 309 | Open reading frame for uracil glycosylase inhibitor (UGI) |
| 310 | Open reading frame encoding Nme2 base editor |
| 311 | Amino acid sequence of Nme2 base editor |
| 312 | Exemplary modified Nme guide sgRNA |
| 313 | Exemplary modified Nme guide sgRNA |
| 314 | Exemplary modified Nme guide sgRNA |
| 315-399 | Guide sequences or guide RNA full or modified sequences (see Table 21) |
| 400 | Exemplary NmeCas9 sgRNA-1 |

### DETAILED DESCRIPTION

The present disclosure provides, e.g., platform methods of contacting a cell with at least two genome editing tools and for multiplex genome editing. The methods provide, for example, multiplex genome editing in a cell without significant cellular side effects. The methods also provide delivering multiple genome editing tools to a cell in fewer steps, allowing for multi-editing within a shorter time period.

In some embodiments, the platform relates to manufacturing methods to prepare cells in vitro for subsequent therapeutic administration to a subject. In some embodiments, the platform relates to multiplex genome editing via simultaneous or sequential administration of lipid nanoparticles (LNPs) comprising at least two genome editing tools. The platform is relevant to any cell type but is particularly advantageous in preparing cells that require multiple genome edits for full therapeutic applicability, e.g., in primary immune cells. The methods may exhibit improved properties as compared to prior delivery technologies; for example, the methods provide efficient delivery of nucleic acids such as the at least two genome editing tools, while providing greater survival and expansion of the cells. As provided herein, the platform methods apply to “a cell” or to “a cell population” (or “population of cells”). When referring to delivery or gene editing methods for “a cell” herein, it is understood that the methods may be used for delivery or gene editing to “a cell population.”

In some embodiments, provided herein is a method of genetically modifying a cell, comprising: (a) contacting the cell with a first genome editing tool, wherein the first genome editing tool comprises a first genomic editor and at least one guide RNA (gRNA) that targets at least one genomic locus and that is cognate to the first genomic editor; and (b) contacting the cell with a second genome editing tool, wherein the second genome editing tool comprises a second genomic editor and at least one gRNA that targets at least one genomic locus and that is cognate to the second genomic editor, wherein the first genomic editor is orthogonal to the second genomic editor, thereby producing at least two genome edits in the cell.

In some embodiments, provided herein is a method of genetically modifying a cell, comprising: (a) contacting the cell with a first genome editing tool comprising a first genomic editor comprising a base editor, and at least one guide RNA (gRNA) that targets at least one genomic locus and that is cognate to the base editor; and (b) contacting the cell with a second genome editing tool comprising a second genomic editor comprising an RNA-guided cleavase, and at least one gRNA that targets at least one genomic locus and that is cognate to the RNA-guided cleavase, wherein the base editor is orthogonal to the RNA-guided cleavase, thereby producing at least two genome edits in the cell.

In some embodiments, provided herein is a method of producing a population of cells comprising edited cells, comprising: (a) contacting the cell with a first genome editing tool comprising a first genomic editor comprising a base editor and at least one guide RNA (gRNA) that targets at least one genomic locus and that is cognate to the base editor; (b) contacting the cell with a second genome editing tool comprising a second genomic editor comprising an RNA-guided cleavase and at least one gRNA that targets at least one genomic locus and that is cognate to the RNA-guided cleavase, wherein the base editor is orthogonal to the RNA-guided cleavase; and (c) culturing the cell, thereby producing the population of cells comprising edited cells comprising at least two genome edits per cell.

In some embodiments, provided herein is a composition, comprising: (a) a first genome editing tool, wherein the first genome editing tool comprises a first genomic editor, and at least one guide RNA (gRNA) that targets at least one genomic locus and that is cognate to the first genomic editor; and (b) a second genome editing tool, wherein the second genome editing tool comprises a second genomic editor, and at least one gRNA that targets at least one genomic locus and that is cognate to the second genomic editor, wherein the first genomic editor is orthogonal to the second genomic editor.

In some embodiments, provided herein is a composition, comprising: (a) a first genome editing tool, wherein the first genome editing tool comprises a first genomic editor comprising a base editor, and at least one guide RNA (gRNA) that targets at least one genomic locus and that is cognate to the base editor; and (b) a second genome editing tool comprising a second genomic editor comprising an RNA-guided cleavase, and at least one gRNA that targets at least one genomic locus and that is cognate to the RNA-guided cleavase, wherein the base editor is orthogonal to the RNA-guided cleavase.

In some embodiments, provided herein is a cell treated in vitro with any method or composition disclosed herein. In some embodiments, provided herein is a cell treated in vivo with any method or composition disclosed herein. In some embodiments, provided herein is a population of cells comprising any cell disclosed herein.

In some embodiments, provided herein is use of any cell, population of cells, or composition disclosed herein for treating cancer. In some embodiments, provided herein is use of any cell, population of cells, or composition disclosed herein for preparation of a medicament for treating cancer.

In some embodiments, provided herein is an engineered cell comprising at least three base edits in at least three genomic loci, and at least one exogenous gene.

In some embodiments, provided herein is a composition comprising: a. a gRNA comprising a guide sequence chosen from: i) SEQ ID NOs: 251-264; ii) at least 17, 18, 19, or 20 contiguous nucleotides of a sequence selected from SEQ ID NOs: 251-264; iii) a guide sequence at least 95%, 90%, or 85% identical to a sequence selected from SEQ ID NOs: 251-264; iv) a sequence that comprises 10 contiguous nucleotides±10 nucleotides of a genomic coordinate listed in Table 5; v) at least 17, 18, 19, or 20 contiguous nucleotides of a sequence from (iv); or vi) a guide sequence that is at least 95%, 90%, or 85% identical to a sequence selected from (v); or b. a nucleic acid encoding a gRNA of (a.).

In some embodiments, provided herein is a method of altering a DNA sequence within an AAVS1 gene, comprising delivering to a cell: a. a gRNA comprising a guide sequence chosen from: i) SEQ ID NOs: 251-264; ii) at least 17, 18, 19, or 20 contiguous nucleotides of a sequence selected from SEQ ID NOs: 251-264; iii) a guide sequence at least 95%, 90%, or 85% identical to a sequence selected from SEQ ID NOs: 251-264; iv) a sequence that comprises 10 contiguous nucleotides±10 nucleotides of a genomic coordinate listed in Table 5; v) at least 17, 18, 19, or 20 contiguous nucleotides of a sequence from (iv); or vi) a guide sequence that is at least 95%, 90%, or 85% identical to a sequence selected from (v); or b. a nucleic acid encoding a gRNA of (a.).

In some embodiments, provided herein is method of immunotherapy comprising administering a composition comprising an engineered cell to a subject, wherein the cell comprises a genomic modification in the AAVS1 gene, wherein the genetic modification comprises an insertion within the genomic coordinates selected from: chr19:55115695-55115715; chr19:55115588-55115608; chr19:55115616-55115636; chr19:55115623-55115643; chr19:55115637-55115657; chr19:55115691-55115711; chr19:55115755-55115775; chr19:55115823-55115843; chr19:55115834-55115854; chr19:55115835-55115855; chr19:55115836-55115856; chr19:55115850-55115870; chr19:55115951-55115971; and chr19:55115949-55115969; or wherein the cell is engineered by delivering to the cell: a. a gRNA comprising a guide sequence chosen from: i) SEQ ID NOs: 251-264; ii) at least 17, 18, 19, or 20 contiguous nucleotides of a sequence selected from SEQ ID NOs: 251-264; iii) a guide sequence at least 95%, 90%, or 85% identical to a sequence selected from SEQ ID NOs: 251-264; iv) a sequence that comprises 10 contiguous nucleotides±10 nucleotides of a genomic coordinate listed in Table 5; v) at least 17, 18, 19, or 20 contiguous nucleotides of a sequence from (iv); or vi) a guide sequence that is at least 95%, 90%, or 85% identical to a sequence selected from (v); or b. a nucleic acid encoding a gRNA of (a.).

In some embodiments, provided herein is an engineered cell comprising a genetic modification in the AAVS1 gene, wherein the genetic modification comprises an insertion within the genomic coordinates chosen from: chr19:55115695-55115715; chr19:55115588-55115608; chr19:55115616-55115636; chr19:55115623-55115643; chr19:55115637-55115657; chr19:55115691-55115711; chr19:55115755-55115775; chr19:55115823-55115843; chr19:55115834-55115854; chr19:55115835-55115855; chr19:55115836-55115856; chr19:55115850-55115870; chr19:55115951-55115971; and chr19:55115949-55115969.

Provided herein are the following numbered embodiments:

Embodiment 1 is a method of genetically modifying a cell, comprising:
(a) contacting the cell with a first genome editing tool, wherein the first genome editing tool comprises a first genomic editor and at least one guide RNA (gRNA) that targets at least one genomic locus and that is cognate to the first genomic editor; and
(b) contacting the cell with a second genome editing tool, wherein the second genome editing tool comprises a second genomic editor and at least one gRNA that targets at least one genomic locus and that is cognate to the second genomic editor, wherein the first genomic editor is orthogonal to the second genomic editor,
thereby producing at least two genome edits in the cell.
   Embodiment 2 is the method of embodiment 1, wherein the first genomic editor or the second genomic editor is delivered to the cell as at least one polypeptide or at least one polynucleotide that encodes the polypeptide.
   Embodiment 3 is the method of embodiment 2, wherein the at least one polynucleotide is at least one mRNA.
   Embodiment 4 is the method of any one of embodiments 1-3, wherein the at least one gRNA is delivered to the cell as at least one polynucleotide that encodes the gRNA.
   Embodiment 5 is the method of any one of embodiments 1-4, wherein the first genomic editor comprises a cleavase, a nickase, a catalytically inactive nuclease, a base editor, optionally a C to T base editor or an A to G base editor, or a fusion protein comprising a DNA polymerase and a nickase.
   Embodiment 6 is the method of any one of embodiments 1-5, wherein the second genomic editor comprises a cleavase, a nickase, a catalytically inactive nuclease, a base editor, optionally a C to T base editor or an A to G base editor, or a fusion protein comprising a DNA polymerase and a nickase.
   Embodiment 7 is the method of any one of embodiments 1-6, wherein one of the first genomic editor and the second genomic editor comprises a base editor, optionally a C to T base editor or an A to G base editor, and the other of the first genomic editor and the second genomic editor comprises a cleavase.
   Embodiment 8 is the method of embodiment 7, further comprising contacting the cell with a nucleic acid encoding an exogenous gene.
   Embodiment 9 is the method of any one of embodiments 1-6, wherein one of the first genomic editor and the second genomic editor comprises a C to T base editor, and the other of the first genomic editor and the second genomic editor comprises an A to G base editor.
   Embodiment 10 is the method of any one of embodiments 1-9, wherein one of the first genomic editor and second genomic editor comprises an N. meningitidis (Nine) RNA-guided nickase or cleavase, and the other of the first genomic editor and the second genomic editor comprises an S. pyogenes (Spy) RNA-guided nickase or cleavase.
   Embodiment 11 is the method of any one of embodiments 1-10, wherein the first genomic editor or the second genomic editor comprises a Cas nuclease.
   Embodiment 12 is the method of embodiment 11, wherein the Cas nuclease is a Class 2 Cas nuclease.
   Embodiment 13 is the method of embodiment 11, wherein the Cas nuclease is a Cas9.
   Embodiment 14 is the method of embodiment 13, wherein the Cas9 is S. pyogenes Cas9 (SpyCas9), S. aureus Cas9 (SauCas9), C. diphtheriae Cas9 (CdiCas9), Streptococcus thermophilus Cas9 (St1Cas9), A. cellulolyticus Cas9 (AceCas9), C. jejuni Cas9 (CjeCas9). R. palustris Cas9 (RpaCas9), R. rubrum Cas9 (RruCas9), A. naeslundii Cas9 (AnaCas9), Francisella novicida Cas9 (FnoCas9), or N. meningitidis (NmeCas9).
   Embodiment 15 is the method of embodiment 13 or embodiment 14, wherein the Cas9 is an Nme1Cas9, an Nme2Cas9, an Nme3Cas9, or SpyCas9.
   Embodiment 16 is a method of genetically modifying a cell, comprising:
(a) contacting the cell with a first genome editing tool comprising a first genomic editor comprising a base editor, and at least one guide RNA (gRNA) that targets at least one genomic locus and that is cognate to the base editor; and
(b) contacting the cell with a second genome editing tool comprising a second genomic editor comprising an RNA-guided cleavase, and at least one gRNA that targets at least one genomic locus and that is cognate to the RNA-guided cleavase, wherein the base editor is orthogonal to the RNA-guided cleavase,
thereby producing at least two genome edits in the cell.
   Embodiment 17 is a method of producing a population of cells comprising edited cells, comprising:
(a) contacting the cell with a first genome editing tool comprising a first genomic editor comprising a base editor and at least one guide RNA (gRNA) that targets at least one genomic locus and that is cognate to the base editor;
(b) contacting the cell with a second genome editing tool comprising a second genomic editor comprising an RNA-guided cleavase and at least one gRNA that targets at least one genomic locus and that is cognate to the RNA-guided cleavase, wherein the base editor is orthogonal to the RNA-guided cleavase; and
(c) culturing the cell, thereby producing the population of cells comprising edited cells comprising at least two genome edits per cell.
   Embodiment 18 is the method of embodiment 16 or 17, wherein the base editor is a C to T base editor, optionally comprising a cytidine deaminase, or is an A to G base editor, optionally comprising an adenosine deaminase.
   Embodiment 19 is the method of any one of embodiments 1-18, wherein one of the at least two genome edits comprises a double-stranded break, and another one of the at least two genome edits comprises a transition (e.g., A to G or C to T) Embodiment 20 is the method of any one of embodiments 1-19, wherein the first genome editing tool or the second genome editing tool is delivered to the cell via electroporation.
   Embodiment 21 is the method of any one of embodiments 1-20, wherein the first genome editing tool or the second genome editing tool is delivered to the cell via at least one lipid nanoparticle (LNP).
   Embodiment 22 is the method of any one of embodiments 1-21, wherein the first genome editing tool or the second genome editing tool is delivered to the cell on at least one vector.
   Embodiment 23 is the method of any one of embodiments 1-22, wherein the first genome editing tool or the second genome editing tool is delivered as at least one nucleic acid encoding the first genome editing tool or the second genome editing tool.
   Embodiment 24 is the method of embodiment 23, wherein the at least one nucleic acid comprises at least one mRNA.
   Embodiment 25 is the method of embodiments 1-24, wherein step (a) and step (b) are performed simultaneously.
   Embodiment 26 is the method of any one of embodiments 1-25, wherein step (a) and step (b) are performed in any order over a time period of about 5 minutes, 10 minutes, 15 minutes, 30 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours.
   Embodiment 27 is the method of any one of embodiments 1-26, wherein each of step (a) and step (b) is independently performed over a time period of about 5 minutes, 10 minutes, 15 minutes, 30 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours.
   Embodiment 28 is the method of any one of embodiments 16-27, wherein the first genome editing tool comprises a uracil glycosylase inhibitor (UGI), and the UGI and the base editor are comprised in a single polypeptide.
   Embodiment 29 is the method of any one of embodiments 16-27, wherein the first genome editing tool comprises a uracil glycosylase inhibitor (UGI), and the UGI and the base editor are comprised in different polypeptides.
   Embodiment 30 is the method of embodiment 28 or 29, wherein the base editor comprises a cytidine deaminase and an RNA-guided nickase.
   Embodiment 31 is the method of embodiment 30, wherein the cytidine deaminase, the RNA-guided nickase, and the UGI are comprised in a single polypeptide.
   Embodiment 32 is the method of embodiment 30, wherein the cytidine deaminase, the RNA-guided nickase, and the UGI are comprised in different polypeptides.
   Embodiment 33 is the method of embodiment 30, wherein the cytidine deaminase and the RNA-guided nickase are comprised in a single polypeptide, and wherein the UGI is comprised in a different polypeptide.
   Embodiment 34 is the method of any one of embodiments 1-33, wherein the first genomic editor comprises an amino acid sequence that is at least 80%, 85%, 90%, 95%, 98%, or 100% identical to SEQ ID NO: 3, 146, or 311.
   Embodiment 35 is the method of any one of embodiments 1-34, wherein the first genomic editor is delivered to the cell as a nucleic acid comprising a nucleotide sequence that is at least 80%, 85%, 90%, 95%, 98%, or 100% identical to SEQ ID NO: 1, and the second genomic editor is delivered to the cell as a nucleic acid comprising a nucleotide sequence that is at least 80%, 85%, 90%, 95%, 98%, or 100% identical to any one of SEQ ID NOs: 180-190.
   Embodiment 36 is the method of any one of embodiments 1-35, wherein the first genomic editor is delivered to the cell as a nucleic acid comprising a nucleotide sequence that is at least 80%, 85%, 90%, 95%, 98%, or 100% identical to SEQ ID NO: 147 or 310, and the second genomic editor is delivered to the cell as a nucleic acid comprising a nucleotide sequence that is at least 80%, 85%, 90%, 95%, 98%, or 100% identical to SEQ ID NO: 293 or 295.
   Embodiment 37 is the method of any one of embodiments 1-33, wherein the first genomic editor or the base editor comprises an amino acid sequence that is at least 80%, 85%, 90%, 95%, 98%, or 100% identical to any one of SEQ ID NOs: 9, 12, 18, and 21.
   Embodiment 38 is the method of any one of embodiments 1-37, wherein the first genomic editor or the base editor comprises a cytidine deaminase, and wherein the cytidine deaminase comprises an amino acid sequence that is at least 80%, 85%, 87%, 90%, 95%, 98%, 99%, or 100% identical to SEQ ID NO: 22.
   Embodiment 39 is the method of embodiment 38, wherein the cytidine deaminase comprises an APOBEC3A deaminase (A3A).
   Embodiment 40 is the method of embodiment 39, wherein the A3A comprises the amino acid sequence of SEQ ID NO: 22 or an amino acid sequence that is at least 87%, 90%, 95%, 98%, or 99% identical to SEQ ID NO: 22.
   Embodiment 41 is the method of embodiment 39 or 40, wherein the A3A is a human A3A.
   Embodiment 42 is the method of any one of embodiments 39-41, wherein the A3A is a wild-type A3A.
   Embodiment 43 is the method of any one of embodiments 1-42, wherein the first genomic editor or the base editor comprises a Cas9 nickase.
   Embodiment 44 is the method of any one of embodiments 1-43, wherein the first genomic editor or the base editor comprises an N. meningitidis (Nine) Cas9 nickase.
   Embodiment 45 is the method of any one of embodiments 1-44, wherein the first genomic editor or the base editor comprises a D16A NmeCas9 nickase, optionally a D16A Nme2Cas9.
   Embodiment 46 is the method of any one of embodiments 1-45, wherein the first genomic editor or the base editor comprises the amino acid sequence of SEQ ID NO: 149.
   Embodiment 47 is the method of any one of embodiments 1-46, wherein the first genomic editor or the base editor comprises a sequence that is at least 80%, 85%, 90%, 95%, 98%, 99% or 100% identical to the amino acid sequence of SEQ ID NO: 146.
   Embodiment 48 is the method of any one of embodiments 1-47, wherein the second genomic editor or the RNA-guided cleavase comprises a Cas9 cleavase.
   Embodiment 49 is the method of any one of embodiments 1-48, wherein the second genomic editor or the RNA-guided cleavase comprises an S. pyogenes (Spy) Cas9 cleavase.
   Embodiment 50 is the method of any one of embodiments 1-49, wherein the second genomic editor or the RNA-guided cleavase comprises an amino acid sequence that is at least 90% identical to SEQ ID NO: 156.
   Embodiment 51 is the method of any one of embodiments 1-50, wherein the second genomic editor or the RNA-guided cleavase comprises the amino acid sequence of SEQ ID NO: 156.
   Embodiment 52 is the method of any one of embodiments 1-43, wherein the first genomic editor or the base editor comprises an S. pyogenes (Spy) Cas9 nickase.
   Embodiment 53 is the method of any one of embodiments 1-43 and 52, wherein the first genomic editor or the base editor comprises a D10A SpyCas9 nickase.
   Embodiment 54 is the method of any one of embodiments 1-43, 52, and 53, wherein the first genomic editor or the base editor comprises the amino acid sequence of any one of SEQ ID NOs: 41, 43, and 45 or an amino acid sequence having at least 80%, 90%, 95%, 98%, or 99% identity to any one of SEQ ID NOs: 41, 43, and 45.
   Embodiment 55 is the method of any one of embodiments 1-43 and 52-54, wherein the first genomic editor or the base editor is delivered to the cell as a nucleic acid comprising the nucleotide sequence of any one of SEQ ID NOs: 42, 44, and 46 or a nucleotide sequence having at least 80%, 90%, 95%, 98%, or 99% identity to any one of SEQ ID NOs: 42, 44, and 46.
   Embodiment 56 is the method of any one of embodiments 1-43 and 52-54, wherein the first genomic editor or the base editor is delivered to the cell as a nucleic acid comprising the nucleotide sequence of any one of SEQ ID NOs: 42, 44, and 46-58.
   Embodiment 57 is the method of any one of embodiments 1-43 and 52-54, wherein the first genomic editor or the base editor is delivered to the cell as a nucleic acid comprising a nucleotide sequence that is at least 80%, 85%, 90%, 95%, 98% or 100% identical to SEQ ID NO: 1.
   Embodiment 58 is the method of any one of embodiments 1-43 and 52-54, wherein the first genomic editor or the base editor is delivered to the cell as a nucleic acid comprising a nucleotide sequence that is at least 80%, 85%, 90%, 95%, 98% or 100% identical to SEQ ID NO: 4.
   Embodiment 59 is the method of any one of embodiments 1-43 and 52-56, wherein the first genomic editor or the base editor comprises an amino acid sequence that is at least 80%, 85%, 90%, 95%, 98% or 100% identical to SEQ ID NO: 148.
   Embodiment 60 is the method of any one of embodiments 1-43 and 52-59, wherein the second genomic editor or the RNA-guided cleavase comprises an N. meningitidis (Nine) Cas9 cleavase.
   Embodiment 61 is the method of any one of embodiments 1-43 and 52-60, wherein the second genomic editor or the RNA-guided cleavase comprises an amino acid sequence that is at least 90%, 91%, 92%, 93%, 94%, 95%, 96%, 97%, 98%, or 99% identical to any one of SEQ ID NOs: 157-167, 191, 198, 205, 212, and 219.
   Embodiment 62 is the method of any one of embodiments 1-43 and 52-61, wherein the second genomic editor or the RNA-guided cleavase comprises the amino acid sequence of any one of SEQ ID NOs: 157-167, 191, 198, 205, 212, and 219.
   Embodiment 63 is the method of any one of embodiments 1-43 and 52-61, wherein the second genomic editor or the RNA-guided cleavase is delivered to the cell as a nucleic acid comprising a nucleotide sequence that is at least 90%, 91%, 92%, 93%, 94%, 95%, 96%, 97%, 98%, or 99% identical to any one of SEQ ID NOs: 168-190, 192-197, 199-204, 206-211, 213-218, and 220-225.
   Embodiment 64 is the method of any one of embodiments 1-43 and 52-61, wherein the second genomic editor or the RNA-guided cleavase is delivered to the cell as a nucleic acid comprising a nucleotide sequence of any one of SEQ ID NOs: 168-190, 192-197, 199-204, 206-211, 213-218, and 220-225.
   Embodiment 65 is the method of any one of embodiments 1-64, wherein at least one gRNA that is cognate to the first genomic editor or the base editor is non-cognate to the second genomic editor or the RNA-guided cleavase.
   Embodiment 66 is the method of any one of embodiments 1-65, wherein at least one gRNA that is cognate to the second genomic editor or the RNA-guided cleavase is non-cognate to the first genomic editor or the base editor.
   Embodiment 67 is the method of any one of embodiments 1-66, wherein the at least one gRNA comprises at least one single guide RNA (sgRNA).
   Embodiment 68 is the method of embodiment 67, wherein the at least one sgRNA comprises a short-single guide RNA (short-sgRNA) comprising a conserved portion of an sgRNA comprising a hairpin region, wherein the hairpin region lacks at least 5-10 nucleotides and wherein the short-sgRNA comprises a 5′ end modification or a 3′ end modification or both.
   Embodiment 69 is the method of any one of embodiments 1-68, wherein the at least one gRNA that is cognate to the first genomic editor or the base editor comprises at least two gRNAs that target at least two different genomic loci.
   Embodiment 70 is the method of any one of embodiments 1-69, wherein the at least one gRNA that is cognate to the second genomic editor or the RNA-guided cleavase comprises at least two gRNAs that target at least two different genomic loci.
   Embodiment 71 is the method of any one of embodiments 1-70, wherein the at least one gRNA that is cognate to the first genomic editor or the base editor comprises at least three gRNAs that target at least three different genomic loci.
   Embodiment 72 is the method of any one of embodiments 1-71, wherein the at least one gRNA that is cognate to the second genomic editor or the RNA-guided cleavase comprises at least three gRNAs that target at least three different genomic loci.
   Embodiment 73 is the method of any one of embodiments 1-72, wherein the at least one gRNA that is cognate to the first genomic editor or the base editor comprises at least four gRNAs that target at least four different genomic loci.
   Embodiment 74 is the method of any one of embodiments 1-73, wherein the at least one gRNA that is cognate to the second genomic editor or the RNA-guided cleavase comprises at least four gRNAs that target at least four different genomic loci.
   Embodiment 75 is the method of any one of embodiments 1-74, wherein the at least one gRNA that is cognate to the first genomic editor or the base editor comprises at least five gRNAs that target at least five different genomic loci.
   Embodiment 76 is the method of any one of embodiments 1-75, wherein the at least one gRNA that is cognate to the second genomic editor or the RNA-guided cleavase comprises at least five gRNAs that target at least five different genomic loci.
   Embodiment 77 is the method of any one of embodiments 1-76, wherein the at least one gRNA that is cognate to the first genomic editor or the base editor comprises at least six gRNAs that target at least six different genomic loci.
   Embodiment 78 is the method of any one of embodiments 1-77, wherein the at least one gRNA that is cognate to the second genomic editor or the RNA-guided cleavase comprises at least six gRNAs that target at least six different genomic loci.
   Embodiment 79 is the method of any one of embodiments 1-78, wherein the at least one gRNA that is cognate to the first genomic editor or the base editor targets one or more genomic loci chosen from the TRBC locus, the HLA-A locus, the HLA-B locus, the CIITA locus, the HLA-DR locus, the HLA-DQ locus, and the HLA-DP locus.
   Embodiment 80 is the method of any one of embodiments 1-79, wherein the at least one gRNA that is cognate to the second genomic editor or the RNA-guided cleavase targets one or more genomic loci chosen from the TRAC locus, the AAVS1 locus, and the CIITA locus.
   Embodiment 81 is the method of any one of embodiments 1-80, wherein
(i) the at least one gRNA that is cognate to the first genomic editor or the base editor comprises a gRNA that targets the HLA-A locus and a gRNA that targets the CIITA locus, and the at least one gRNA that is cognate to the second genomic editor or the RNA-guided cleavase comprises a gRNA that targets the TRAC locus;
(ii) the at least one gRNA that is cognate to the first genomic editor or the base editor comprises a gRNA that targets the TRBC locus, a gRNA that targets the HLA-A locus, and a gRNA that targets the CIITA locus, and the at least one gRNA that is cognate to the second genomic editor or the RNA-guided cleavase comprises a gRNA that targets the TRAC locus;
(iii) the at least one gRNA that is cognate to the first genomic editor or the base editor comprises a gRNA that targets the HLA-A locus, a gRNA that targets the HLA-B locus, and a gRNA that targets the CIITA locus, and the at least one gRNA that is cognate to the second genomic editor or the RNA-guided cleavase comprises a gRNA that targets the TRAC locus;
(iv) the at least one gRNA that is cognate to the first genomic editor or the base editor comprises a gRNA that targets the TRBC locus, a gRNA that targets the HLA-A locus, a gRNA that targets the HLA-B locus, and a gRNA that targets the CIITA locus, and the at least one gRNA that is cognate to the second genomic editor or the RNA-guided cleavase comprises a gRNA that targets the TRAC locus;
(v) the at least one gRNA that is cognate to the first genomic editor or the base editor comprises a gRNA that targets the HLA-A locus and a gRNA that targets the HLA-DR locus, the HLA-DQ locus, or the HLA-DP locus, and the at least one gRNA that is cognate to the second genomic editor or the RNA-guided cleavase comprises a gRNA that targets the TRAC locus;
(vi) the at least one gRNA that is cognate to the first genomic editor or the base editor comprises a gRNA that targets the TRBC locus, a gRNA that targets the HLA-A locus, and a gRNA that targets the HLA-DR locus, the HLA-DQ locus, or the HLA-DP locus, and the at least one gRNA that is cognate to the second genomic editor or the RNA-guided cleavase comprises a gRNA that targets the TRAC locus;
(vii) the at least one gRNA that is cognate to the first genomic editor or the base editor comprises a gRNA that targets the HLA-A locus, a gRNA that targets the HLA-B locus, and a gRNA that targets the HLA-DR locus, the HLA-DQ locus, or the HLA-DP locus, and the at least one gRNA that is cognate to the second genomic editor or the RNA-guided cleavase comprises a gRNA that targets the TRAC locus;
(viii) the at least one gRNA that is cognate to the first genomic editor or the base editor comprises a gRNA that targets the TRBC locus, a gRNA that targets the HLA-A locus, a gRNA that targets the HLA-B locus, and a gRNA that targets the HLA-DR locus, the HLA-DQ locus, or the HLA-DP locus, and the at least one gRNA that is cognate to the second genomic editor or the RNA-guided cleavase comprises a gRNA that targets the TRAC locus;
(ix) the at least one gRNA that is cognate to the first genomic editor or the base editor comprises a gRNA that targets the TRAC locus, a gRNA that targets the TRBC locus, a gRNA that targets the CIITA locus, and a gRNA that targets the HLA-A locus, and the at least one gRNA that is cognate to the second genomic editor or the RNA-guided cleavase comprises a gRNA that targets the TRAC locus;
(x) the at least one gRNA that is cognate to the first genomic editor or the base editor comprises a gRNA that targets the TRBC locus, a gRNA that targets the HLA-A locus, and a gRNA that targets the CIITA locus, and the at least one gRNA that is cognate to the second genomic editor or the RNA-guided cleavase comprises a gRNA that targets the AAVS1 locus;
(xi) the at least one gRNA that is cognate to the first genomic editor or the base editor comprises a gRNA that targets the TRBC locus, a gRNA that targets the HLA-A locus, a gRNA that targets the HLA-B locus, and a gRNA that targets the CIITA locus, and the at least one gRNA that is cognate to the second genomic editor or the RNA-guided cleavase comprises a gRNA that targets the AAVS1 locus;
(xii) the at least one gRNA that is cognate to the first genomic editor or the base editor comprises a gRNA that targets the TRBC locus, a gRNA that targets the HLA-A locus, and a gRNA that targets the HLA-DR locus, the HLA-DQ locus, or the HLA-DP locus, and the at least one gRNA that is cognate to the second genomic editor or the RNA-guided cleavase comprises a gRNA that targets the AAVS1 locus; or
(xiii) the at least one gRNA that is cognate to the first genomic editor or the base editor comprises a gRNA that targets the TRBC locus, a gRNA that targets the HLA-A locus, a gRNA that targets the HLA-B locus, and a gRNA that targets the HLA-DR locus, the HLA-DQ locus, or the HLA-DP locus, and the at least one gRNA that is cognate to the second genomic editor or the RNA-guided cleavase comprises a gRNA that targets the AAVS1 locus.
   Embodiment 82 is the method of any one of embodiments 1-81, further comprising contacting the cell with a nucleic acid encoding an exogenous gene for insertion into the TRAC or AAVS1 locus.
   Embodiment 83 is the method of embodiment 82, wherein in any one of subparts (i)-(ix), the at least one gRNA that is cognate to the second genomic editor or the RNA-guided cleavase comprises a further gRNA that targets the AAVS1 locus.
   Embodiment 84 is the method of embodiment 82, wherein in any one of subparts (x)-(xiii), the at least one gRNA that is cognate to the second genomic editor or the RNA-guided cleavase comprises a further gRNA that targets the TRAC locus.
   Embodiment 85 is the method of embodiment 84, wherein the cell is contacted with the further gRNA that targets the AAVS1 locus after the cell is contacted with the gRNA that targets the TRAC locus.
   Embodiment 86 is the method of embodiment 85, wherein the cell is contacted with the further gRNA that targets the TRAC locus after the cell is contacted with the gRNA that targets the AAVS1 locus.
   Embodiment 87 is a composition, comprising:
(a) a first genome editing tool, wherein the first genome editing tool comprises a first genomic editor, and at least one guide RNA (gRNA) that targets at least one genomic locus and that is cognate to the first genomic editor; and
(b) a second genome editing tool, wherein the second genome editing tool comprises a second genomic editor, and at least one gRNA that targets at least one genomic locus and that is cognate to the second genomic editor, wherein the first genomic editor is orthogonal to the second genomic editor.
   Embodiment 88 is the composition of embodiment 87, wherein the first genomic editor or the second genomic editor comprises at least one polypeptide or at least one mRNA.
   Embodiment 89 is the composition of embodiment 87 or 88, wherein the at least one gRNA comprises at least one polynucleotide that encodes the gRNA.
   Embodiment 90 is the composition of any one of embodiments 87-89, wherein the first genomic editor comprises a cleavase, a nickase, a catalytically inactive nuclease, a base editor, optionally a C to T base editor or an A to G base editor, or a fusion protein comprising a DNA polymerase and a nickase.
   Embodiment 91 is the composition of any one of embodiments 87-90, wherein the second genomic editor comprises a cleavase, a nickase, a catalytically inactive nuclease, a base editor, optionally a C to T base editor or an A to G base editor, or a fusion protein comprising a DNA polymerase and a nickase.
   Embodiment 92 is the composition of any one of embodiments 87-91, wherein one of the first genomic editor and the second genomic editor comprises a base editor, optionally a C to T base editor or an A to G base editor, and the other of the first genomic editor and the second genomic editor comprises a cleavase.
   Embodiment 93 is the composition of embodiment 92, further comprising a nucleic acid encoding an exogenous gene.
   Embodiment 94 is the composition of any one of embodiments 87-91, wherein one of the first genomic editor and the second genomic editor comprises a C to T base editor, and the other of the first genomic editor and the second genomic editor comprises an A to G base editor.
   Embodiment 95 is the composition of any one of embodiments 87-94, wherein one of the first genomic editor and the second genomic editor comprises an N. meningitidis (Nine) RNA-guided nickase, and the other of the first genomic editor and the second genomic editor comprises an S. pyogenes (Spy) RNA-guided nickase.
   Embodiment 96 is the composition of any one of embodiments 87-95, wherein the first genomic editor or the second genomic editor is a Cas nuclease.
   Embodiment 97 is the composition of embodiment 96, wherein the Cas nuclease is a Class 2 Cas nuclease.
   Embodiment 98 is the composition of embodiment 96, wherein the Cas nuclease is a Cas9.
   Embodiment 99 is the composition of embodiment 98, wherein the Cas9 is S. pyogenes Cas9 (SpyCas9), S. aureus Cas9 (SauCas9), C. diphtheriae Cas9 (CdiCas9), Streptococcus thermophilus Cas9 (St1Cas9), A. cellulolyticus Cas9 (AceCas9), C. jejuni Cas9 (CjeCas9). R. palustris Cas9 (RpaCas9), R. rubrum Cas9 (RruCas9), A. naeslundii Cas9 (AnaCas9), Francisella novicida Cas9 (FnoCas9), or N. meningitidis (NmeCas9).
   Embodiment 100 is the composition of embodiment 98 or 99, wherein the Cas9 is an Nme1Cas9, an Nme2Cas9, an Nme3Cas9, or SpyCas9.
   Embodiment 101 is a composition, comprising:
(a) a first genome editing tool, wherein the first genome editing tool comprises a first genomic editor comprising a base editor, and at least one guide RNA (gRNA) that targets at least one genomic locus and that is cognate to the base editor; and
(b) a second genome editing tool comprising a second genomic editor comprising an RNA-guided cleavase, and at least one gRNA that targets at least one genomic locus and that is cognate to the RNA-guided cleavase, wherein the base editor is orthogonal to the RNA-guided cleavase.
   Embodiment 102 is the composition of embodiment 101, wherein the base editor is a C to T base editor, optionally comprising a cytidine deaminase, or is an A to G base editor, optionally comprising an adenosine deaminase.
   Embodiment 103 is the composition of any one of embodiments 87-102, wherein the first genome editing tool or the second genome editing tool is delivered to a cell via electroporation.
   Embodiment 104 is the composition of any one of embodiments 87-103, wherein the first genome editing tool or the second genome editing tool is contained in at least one lipid nanoparticle (LNP).
   Embodiment 105 is the composition of any one of embodiments 87-104, wherein the first genome editing tool or the second genome editing tool comprises at least one vector.
   Embodiment 106 is the composition of any one of embodiments 87-105, wherein the first genome editing tool or the second genome editing tool comprises at least one polypeptide or at least one nucleic acid encoding the first genome editing tool or the second genome editing tool.
   Embodiment 107 is the composition of any one of embodiments 87-106, wherein the first genome editing tool comprises at least one polypeptide comprising the first genome editing tool or at least one nucleic acid encoding the first genome editing tool.
   Embodiment 108 is the composition of any one of embodiments 87-107, wherein the second genome editing tool comprises at least one polypeptide comprising the second genome editing tool or at least one nucleic acid encoding the second genome editing tool.
   Embodiment 109 is the composition of any one of embodiments 106-108, wherein the at least one nucleic acid comprises at least one mRNA.
   Embodiment 110 is the composition of any one of embodiments 101-109, wherein the first genome editing tool comprises a uracil glycosylase inhibitor (UGI), and the UGI and the base editor are comprised in a single polypeptide.
   Embodiment 111 is the composition of any one of embodiments 101-109, wherein the first genome editing tool comprises a uracil glycosylase inhibitor (UGI), and the UGI and the base editor are comprised in different polypeptides.
   Embodiment 112 is the composition of embodiment 110 or 111, wherein the base editor comprises a cytidine deaminase and an RNA-guided nickase.
   Embodiment 113 is the composition of embodiment 112, wherein the cytidine deaminase, the RNA-guided nickase, and the UGI are comprised in a single polypeptide.
   Embodiment 114 is the composition of embodiment 112, wherein the cytidine deaminase, the RNA-guided nickase, and the UGI are comprised in different polypeptides.
   Embodiment 115 is the composition of embodiment 112, wherein the cytidine deaminase and the RNA-guided nickase are comprised in a single polypeptide, and wherein the UGI is comprised in a different polypeptide.
   Embodiment 116 is the composition of any one of embodiments 87-115, wherein the first genomic editor comprises an amino acid sequence that is at least 80%, 85%, 90%, 95%, 98%, or 100% identical to SEQ ID NO: 3, 146, or 311.
   Embodiment 117 is the composition of any one of embodiments 87-116, wherein the first genomic editor is delivered to a cell as a nucleic acid comprising a nucleotide sequence that is at least 80%, 85%, 90%, 95%, 98%, or 100% identical to SEQ ID NO: 1, and the second genomic editor is delivered to a cell as a nucleic acid comprising a nucleotide sequence that is at least 80%, 85%, 90%, 95%, 98%, or 100% identical to any one of SEQ ID NOs: 180-190.
   Embodiment 118 is the composition of any one of embodiments 87-117, wherein the first genomic editor is delivered to a cell as a nucleic acid comprising a nucleotide sequence that is at least 80%, 85%, 90%, 95%, 98%, or 100% identical to SEQ ID NO: 147 or 310, and the second genomic editor is delivered to a cell as a nucleic acid comprising a nucleotide sequence that is at least 80%, 85%, 90%, 95%, 98%, or 100% identical to SEQ ID NO: 293 or 295.
   Embodiment 119 is the composition of any one of embodiments 87-115, wherein the first genomic editor or the base editor comprises an amino acid sequence that is at least 80%, 85%, 90%, 95%, 98%, or 100% identical to any one of SEQ ID NOs: 9, 12, 18, and 21.
   Embodiment 120 is the composition of any one of embodiments 87-119, wherein the first genomic editor or the base editor comprises a cytidine deaminase, and wherein the cytidine deaminase comprises an amino acid sequence that is at least 80%, 85%, 87%, 90%, 95%, 98%, 99%, or 100% identical to SEQ ID NO: 22.
   Embodiment 121 is the composition of embodiment 120, wherein the cytidine deaminase comprises an APOBEC3A deaminase (A3A).
   Embodiment 122 is the composition of embodiment 121, wherein the A3A comprises the amino acid sequence of SEQ ID NO: 22 or an amino acid sequence that is at least 87%, 90%, 95%, 98%, or 99% identical to SEQ ID NO: 22.
   Embodiment 123 is the composition of embodiment 121 or 122, wherein the A3A is a human A3A.
   Embodiment 124 is the composition of any one of embodiments 121-123, wherein the A3A is a wild-type A3A.
   Embodiment 125 is the composition of any one of embodiments 87-124, wherein the first genomic editor or the base editor comprises a Cas9 nickase.
   Embodiment 126 is the composition of any one of embodiments 87-125, wherein the first genomic editor or the base editor comprises an N. meningitidis (Nine) Cas9 nickase.
   Embodiment 127 is the composition of any one of embodiments 87-126, wherein the first genomic editor or the base editor comprises a D16A NmeCas9 nickase, optionally a D16A Nme2Cas9.
   Embodiment 128 is the composition of any one of embodiments 87-127, wherein the first genomic editor or the base editor comprises the amino acid sequence of SEQ ID NO: 149.
   Embodiment 129 is the composition of any one of embodiments 87-128, wherein the first genomic editor or the base editor comprises a sequence that is at least 80%, 85%, 90%, 95%, 98%, 99% or 100% identical to the amino acid sequence of SEQ ID NO: 146.
   Embodiment 130 is the composition of any one of embodiments 87-129, wherein the second genomic editor or the RNA-guided cleavase comprises a Cas9 cleavase.
   Embodiment 131 is the composition of any one of embodiments 87-130, wherein the second genomic editor or the RNA-guided cleavase comprises an S. pyogenes (Spy) Cas9 cleavase.
   Embodiment 132 is the composition of any one of embodiments 87-131, wherein the second genomic editor or the RNA-guided cleavase comprises an amino acid sequence that is at least 90% identical to SEQ ID NO: 156.
   Embodiment 133 is the composition of any one of embodiments 87-132, wherein the second genomic editor or the RNA-guided cleavase comprises the amino acid sequence of SEQ ID NO: 156.
   Embodiment 134 is the composition of any one of embodiments 87-125, wherein the first genomic editor or the base editor comprises an S. pyogenes (Spy) Cas9 nickase.
   Embodiment 135 is the composition of any one of embodiments 87-125 and 134, wherein the first genomic editor or the base editor comprises a D10A SpyCas9 nickase.
   Embodiment 136 is the composition of any one of embodiments 87-125, 134, and 135, wherein the first genomic editor or the base editor comprises the amino acid sequence of any one of SEQ ID NOs: 41, 43, and 45 or an amino acid sequence having at least 80%, 90%, 95%, 98%, or 99% identity to any one of SEQ ID NOs: 41, 43, and 45.
   Embodiment 137 is the composition of any one of embodiments 87-125 and 134-136, wherein the first genomic editor or the base editor is delivered to a cell as a nucleic acid comprising the nucleotide sequence of any one of SEQ ID NOs: 42, 44, and 46 or a nucleotide sequence having at least 80%, 90%, 95%, 98%, or 99% identity to any one of SEQ ID NOs: 42, 44, and 46.
   Embodiment 138 is the composition of any one of embodiments 87-125 and 134-137, wherein the first genomic editor or the base editor is delivered to a cell as a nucleic acid comprising the nucleotide sequence of any one of SEQ ID NOs: 42, 44, and 46-58.
   Embodiment 139 is the composition of any one of embodiments 87-125 and 134-138, wherein the first genomic editor or the base editor is delivered to a cell as a nucleic acid comprising a nucleotide sequence that is at least 80%, 85%, 90%, 95%, 98% or 100% identical to SEQ ID NO: 1.
   Embodiment 140 is the composition of any one of embodiments 87-125 and 134-138, wherein the first genomic editor or the base editor is delivered to a cell as a nucleic acid comprising a nucleotide sequence that is at least 80%, 85%, 90%, 95%, 98% or 100% identical to SEQ ID NO: 4.
   Embodiment 141 is the composition of any one of embodiments 87-125 and 134-138, wherein the first genomic editor or the base editor comprises an amino acid sequence that is at least 80%, 85%, 90%, 95%, 98% or 100% identical to SEQ ID NO: 148.
   Embodiment 142 is the composition of any one of embodiments 87-125 and 134-141, wherein the second genomic editor or the RNA-guided cleavase comprises an N. meningitidis (Nine) Cas9 cleavase.
   Embodiment 143 is the composition of any one of embodiments 87-125 and 134-142, wherein the second genomic editor or the RNA-guided cleavase comprises an amino acid sequence that is at least 90%, 91%, 92%, 93%, 94%, 95%, 96%, 97%, 98%, or 99% identical to any one of SEQ ID NOs: 157-167, 191, 198, 205, 212, and 219.
   Embodiment 144 is the composition of any one of embodiments 87-124 and 134-143, wherein the second genomic editor or the RNA-guided cleavase comprises the amino acid sequence of any one of SEQ ID NOs: 157-167, 191, 198, 205, 212, and 219.
   Embodiment 145 is the composition of any one of embodiments 87-124 and 134-144, wherein the second genomic editor or the RNA-guided cleavase is delivered to a cell as a nucleic acid comprising a nucleotide sequence that is at least 90%, 91%, 92%, 93%, 94%, 95%, 96%, 97%, 98%, or 99% identical to any one of SEQ ID NOs: 168-190, 192-197, 199-204, 206-211, 213-218, and 220-225.
   Embodiment 146 is the composition of any one of embodiments 87-124 and 134-144, wherein the second genomic editor or the RNA-guided cleavase is delivered to the cell as a nucleic acid comprising a nucleotide sequence of any one of SEQ ID NOs: 168-190, 192-197, 199-204, 206-211, 213-218, and 220-225.
   Embodiment 147 is the composition of any one of embodiments 87-146, wherein the at least one gRNA that is cognate to the first genomic editor or the base editor is non-cognate to the second genomic editor or the RNA-guided cleavase.
   Embodiment 148 is the composition of any one of embodiments 87-147, wherein the at least one gRNA that is cognate to the second genomic editor or the RNA-guided cleavase is non-cognate to the first genomic editor or the base editor.
   Embodiment 149 is the composition of any one of embodiments 87-148, wherein the at least one gRNA comprises at least one single guide RNA (sgRNA).
   Embodiment 150 is the composition of embodiment 149, wherein the at least one sgRNA comprises a short-single guide RNA (short-sgRNA) comprising a conserved portion of an sgRNA comprising a hairpin region, wherein the hairpin region lacks at least 5-10 nucleotides and wherein the short-sgRNA comprises a 5′ end modification or a 3′ end modification or both.
   Embodiment 151 is the composition of any one of embodiments 87-150, wherein the at least one gRNA that is cognate to the first genomic editor or the base editor comprises at least two gRNAs that target at least two different genomic loci.
   Embodiment 152 is the composition of any one of embodiments 87-151, wherein the at least one gRNA that is cognate to the second genomic editor or the RNA-guided cleavase comprises at least two gRNAs that target at least two different genomic loci.
   Embodiment 153 is the composition of any one of embodiments 87-152, wherein the at least one gRNA that is cognate to the first genomic editor or the base editor comprises at least three gRNAs that target at least three different genomic loci.
   Embodiment 154 is the composition of any one of embodiments 87-153, wherein the at least one gRNA that is cognate to the second genomic editor or the RNA-guided cleavase comprises at least three gRNAs that target at least three different genomic loci.
   Embodiment 155 is the composition of any one of embodiments 87-154, wherein the at least one gRNA that is cognate to the first genomic editor or the base editor comprises at least four gRNAs that target at least four different genomic loci.
   Embodiment 156 is the composition of any one of embodiments 87-155, wherein the at least one gRNA that is cognate to the second genomic editor or the RNA-guided cleavase comprises at least four gRNAs that target at least four different genomic loci.
   Embodiment 157 is the composition of any one of embodiments 87-156, wherein the at least one gRNA that is cognate to the first genomic editor or the base editor comprises at least five gRNAs that target at least five different genomic loci.
   Embodiment 158 is the composition of any one of embodiments 87-157, wherein the at least one gRNA that is cognate to the second genomic editor or the RNA-guided cleavase comprises at least five gRNAs that target at least five different genomic loci.
   Embodiment 159 is the composition of any one of embodiments 87-158, wherein the at least one gRNA that is cognate to the first genomic editor or the base editor comprises at least six gRNAs that target at least six different genomic loci.
   Embodiment 160 is the composition of any one of embodiments 87-159, wherein the at least one gRNA that is cognate to the second genomic editor or the RNA-guided cleavase comprises at least six gRNAs that target at least six different genomic loci.
   Embodiment 161 is the composition of any one of embodiments 151-160, wherein the first genomic editor and one, two, three, four, five, or six of the at least one gRNA that are cognate to the first genomic editor or the base editor and target different genomic loci are contained in a same lipid nanoparticle (LNP).
   Embodiment 162 is the composition of any one of embodiments 87-161, wherein the at least one gRNA that is cognate to the first genomic editor or the base editor targets one or more genomic loci chosen from the TRBC locus, the HLA-A locus, the HLA-B locus, the CIITA locus, the HLA-DR locus, the HLA-DQ locus, and the HLA-DP locus.
   Embodiment 163 is the composition of any one of embodiments 87-162, wherein the at least one gRNA that is cognate to the second genomic editor or the RNA-guided cleavase targets one or more genomic loci chosen from the TRAC locus, the AAVS1 locus, and the CIITA locus.
   Embodiment 164 is the composition of any one of the embodiments 87-163, wherein
(i) the at least one gRNA that is cognate to the first genomic editor or the base editor comprises a gRNA that targets the HLA-A locus and a gRNA that targets the CIITA locus, and the at least one gRNA that is cognate to the second genomic editor or the RNA-guided cleavase comprises a gRNA that targets the TRAC locus;
(ii) the at least one gRNA that is cognate to the first genomic editor or the base editor comprises a gRNA that targets the TRBC locus, a gRNA that targets the HLA-A locus, and a gRNA that targets the CIITA locus, and the at least one gRNA that is cognate to the second genomic editor or the RNA-guided cleavase comprises a gRNA that targets the TRAC locus;
(iii) the at least one gRNA that is cognate to the first genomic editor or the base editor comprises a gRNA that targets the HLA-A locus, a gRNA that targets the HLA-B locus, and a gRNA that targets the CIITA locus, and the at least one gRNA that is cognate to the second genomic editor or the RNA-guided cleavase comprises a gRNA that targets the TRAC locus;
(iv) the at least one gRNA that is cognate to the first genomic editor or the base editor comprises a gRNA that targets the TRBC locus, a gRNA that targets the HLA-A locus, a gRNA that targets the HLA-B locus, and a gRNA that targets the CIITA locus, and the at least one gRNA that is cognate to the second genomic editor or the RNA-guided cleavase comprises a gRNA that targets the TRAC locus;
(v) the at least one gRNA that is cognate to the first genomic editor or the base editor comprises a gRNA that targets the HLA-A locus and a gRNA that targets the HLA-DR locus, the HLA-DQ locus, or the HLA-DP locus, and the at least one gRNA that is cognate to the second genomic editor or the RNA-guided cleavase comprises a gRNA that targets the TRAC locus;
(vi) the at least one gRNA that is cognate to the first genomic editor or the base editor comprises a gRNA that targets the TRBC locus, a gRNA that targets the HLA-A locus, and a gRNA that targets the HLA-DR locus, the HLA-DQ locus, or the HLA-DP locus, and the at least one gRNA that is cognate to the second genomic editor or the RNA-guided cleavase comprises a gRNA that targets the TRAC locus;
(vii) the at least one gRNA that is cognate to the first genomic editor or the base editor comprises a gRNA that targets the HLA-A locus, a gRNA that targets the HLA-B locus, and a gRNA that targets the HLA-DR locus, the HLA-DQ locus, or the HLA-DP locus, and the at least one gRNA that is cognate to the second genomic editor or the RNA-guided cleavase comprises a gRNA that targets the TRAC locus;
(viii) the at least one gRNA that is cognate to the first genomic editor or the base editor comprises a gRNA that targets the TRBC locus, a gRNA that targets the HLA-A locus, a gRNA that targets the HLA-B locus, and a gRNA that targets the HLA-DR locus, the HLA-DQ locus, or the HLA-DP locus, and the at least one gRNA that is cognate to the second genomic editor or the RNA-guided cleavase comprises a gRNA that targets the TRAC locus;
(ix) the at least one gRNA that is cognate to the first genomic editor or the base editor comprises a gRNA that targets the TRAC locus, a gRNA that targets the TRBC locus, a gRNA that targets the CIITA locus, and a gRNA that targets the HLA-A locus, and the at least one gRNA that is cognate to the second genomic editor or the RNA-guided cleavase comprises a gRNA that targets the TRAC locus;
(x) the at least one gRNA that is cognate to the first genomic editor or the base editor comprises a gRNA that targets the TRBC locus, a gRNA that targets the HLA-A locus, and a gRNA that targets the CIITA locus, and the at least one gRNA that is cognate to the second genomic editor or the RNA-guided cleavase comprises a gRNA that targets the AAVS1 locus;
(xi) the at least one gRNA that is cognate to the first genomic editor or the base editor comprises a gRNA that targets the TRBC locus, a gRNA that targets the HLA-A locus, a gRNA that targets the HLA-B locus, and a gRNA that targets the CIITA locus, and the at least one gRNA that is cognate to the second genomic editor or the RNA-guided cleavase comprises a gRNA that targets the AAVS1 locus;
(xii) the at least one gRNA that is cognate to the first genomic editor or the base editor comprises a gRNA that targets the TRBC locus, a gRNA that targets the HLA-A locus, and a gRNA that targets the HLA-DR locus, the HLA-DQ locus, or the HLA-DP locus, and the at least one gRNA that is cognate to the second genomic editor or the RNA-guided cleavase comprises a gRNA that targets the AAVS1 locus; or
(xiii) the at least one gRNA that is cognate to the first genomic editor or the base editor comprises a gRNA that targets the TRBC locus, a gRNA that targets the HLA-A locus, a gRNA that targets the HLA-B locus, and a gRNA that targets the HLA-DR locus, the HLA-DQ locus, or the HLA-DP locus, and the at least one gRNA that is cognate to the second genomic editor or the RNA-guided cleavase comprises a gRNA that targets the AAVS1 locus.
   Embodiment 165 is the composition of any one of embodiments 87-164, further comprising a nucleic acid encoding an exogenous gene for insertion into the TRAC or AAVS1 locus.
   Embodiment 166 is the composition of embodiment 164, wherein in any one of subparts (i)-(ix), the at least one gRNA that is cognate to the second genomic editor or the RNA-guided cleavase comprises a further gRNA that targets the AAVS1 locus.
   Embodiment 167 is the composition of embodiment 164, wherein in any one of subparts (x)-(xiii), the at least one gRNA that is cognate to the second genomic editor or the RNA-guided cleavase comprises a further gRNA that targets the TRAC locus.
   Embodiment 168 is the method or composition of any one of embodiments 1, 16, 17, 87, and 101, wherein the first genome editing tool, the second genome editing tool, and the gRNAs are collectively contained in: (i) a first lipid nanoparticle (LNP) comprising the second genomic editor and a first gRNA, (ii) a second LNP comprising the first genomic editor or the base editor, (iii) a third LNP comprising a uracil glycosylase inhibitor (UGI), (iv) a fourth LNP comprising a second gRNA, (v) a fifth LNP comprising a third gRNA, and (vi) a sixth LNP comprising a fourth gRNA.
   Embodiment 169 is the method or composition of any one of embodiments 1, 16, 17, 87, and 101, wherein the first genome editing tool, the second genome editing tool, and the gRNAs are collectively contained in: (i) a first lipid nanoparticle (LNP) comprising the second genomic editor and a first gRNA, (ii) a second LNP comprising the first genomic editor or the base editor, (iii) a third LNP comprising a uracil glycosylase inhibitor (UGI), (iv) a fourth LNP comprising a second gRNA and a third gRNA, and (v) a fifth LNP comprising a fourth gRNA.
   Embodiment 170 is the method or composition of any one of embodiments 1, 16, 17, 87, and 101, wherein the first genome editing tool, the second genome editing tool, and the gRNAs are collectively contained in: (i) a first lipid nanoparticle (LNP) comprising the second genomic editor and a first gRNA, (ii) a second LNP comprising the first genomic editor or the base editor and comprising a uracil glycosylase inhibitor (UGI), (iii) a third LNP comprising a second gRNA, (iv) a fourth LNP comprising a third gRNA, and (v) a fifth LNP comprising a fourth gRNA.
   Embodiment 171 is the method or composition of any one of embodiments 1, 16, 17, 87, and 101, wherein the first genome editing tool, the second genome editing tool, and the gRNAs are collectively contained in: (i) a first lipid nanoparticle (LNP) comprising the second genomic editor and a first gRNA, (ii) a second LNP comprising the first genomic editor or the base editor and comprising a uracil glycosylase inhibitor (UGI), (iii) a third LNP comprising a second gRNA and a third gRNA, and (iv) a fourth LNP comprising a fourth gRNA.
   Embodiment 172 is the method or composition of any one of embodiments 1, 16, 17, 87, and 101, wherein the first genome editing tool, the second genome editing tool, and the gRNAs are collectively contained in: (i) a first lipid nanoparticle (LNP) comprising the second genomic editor and a first gRNA, (ii) a second LNP comprising the first genomic editor or the base editor, (iii) a third LNP comprising a uracil glycosylase inhibitor (UGI), (iv) a fourth LNP comprising a second gRNA, a third gRNA, and a fourth gRNA.
   Embodiment 173 is the method or composition of any one of embodiments 1, 16, 17, 87, and 101, wherein the first genome editing tool, the second genome editing tool, and the gRNAs are collectively contained in: (i) a first lipid nanoparticle (LNP) comprising the second genomic editor and a first gRNA, (ii) a second LNP comprising a uracil glycosylase inhibitor (UGI), (iii) a third LNP comprising the first genomic editor or the base editor and comprising a second gRNA, (iv) a fourth LNP comprising the first genomic editor or the base editor and comprising a third gRNA, and (v) a fifth LNP comprising the first genomic editor or the base editor and comprising a fourth gRNA.
   Embodiment 174 is the method or composition of any one of embodiments 1, 16, 17, 87, and 101, wherein the first genome editing tool, the second genome editing tool, and the gRNAs are collectively contained in: (i) a first lipid nanoparticle (LNP) comprising the second genomic editor and a first gRNA, (ii) a second LNP comprising a uracil glycosylase inhibitor (UGI), (iii) a third LNP comprising the first genomic editor or the base editor and comprising a second gRNA and a third gRNA, and (iv) a fourth LNP comprising the first genomic editor or the base editor and comprising a fourth gRNA.
   Embodiment 175 is the method or composition of any one of embodiments 168-174, wherein the first genome editing tool, the second genome editing tool, and the gRNAs are collectively contained in the first through fourth LNPs, the first through fifth LNPs, or the first through sixth LNPs, and in one or more additional LNP comprising a fifth gRNA.
   Embodiment 176 is the method or composition of embodiment 175, wherein the one or more additional LNP further comprises a sixth gRNA.
   Embodiment 177 is the method or composition of embodiment 176, wherein the one or more additional LNP further comprises a seventh gRNA.
   Embodiment 178 is the method or composition of embodiment 177, wherein the one or more additional LNP further comprises an eighth gRNA.
   Embodiment 179 is the method or composition of embodiment 178, wherein the one or more additional LNP further comprises a ninth gRNA.
   Embodiment 180 is the method or composition of embodiment 179, wherein the one or more additional LNP further comprises a tenth gRNA.
   Embodiment 181 is the method or composition of any one of embodiments 168-180, wherein the second genomic editor comprises an S. pyogenes (Spy) Cas9 cleavase, the first genomic editor or the base editor comprises an N. meningitidis (Nine) Cas9 nickase, the first gRNA targets the TRAC locus, the second gRNA targets the HLA-A locus, the third gRNA targets the CIITA locus, the fourth gRNA targets the HLA-B locus, the fifth gRNA targets the TRBC locus and the one or more additional gRNAs each targets a locus different from the TRAC locus, the HLA-A locus, the HLA-B locus, the CIITA locus, and the TRBC locus.
   Embodiment 182 is the method or composition of embodiment 181, wherein the first gRNA comprises the sequence of SEQ ID NO: 374 or 378 or a sequence at least 95%, 90%, or 85% identical to SEQ ID NO: 374 or 378, wherein the second gRNA comprises the sequence of SEQ ID NO: 366 or 370 or a sequence at least 95%, 90%, or 85% identical to SEQ ID NO: 366 or 370, wherein the third gRNA comprises the sequence of SEQ ID NO: 345 or 384 or a sequence at least 95%, 90%, or 85% identical to SEQ ID NO: 345 or 384, and wherein the fourth gRNA comprises the sequence of SEQ ID NO: 363 or a sequence at least 95%, 90%, or 85% identical to SEQ ID NO: 363.
   Embodiment 183 is the method or composition of any one of embodiments 1-167, wherein the first genome editing tool, the second genome editing tool, and the gRNAs are collectively contained in at least 2, 3, 4, 5, 6, 7, 8, 9, or 10 distinct lipid nanoparticles (LNP) each comprising a distinct nucleic acid component.
   Embodiment 184 is the method or composition of embodiment 183, wherein the first genome editing tool, the second genome editing tool, and the gRNAs are collectively contained in 4, 5, 6, or 7 distinct lipid nanoparticles (LNP) each comprising a distinct nucleic acid component.
   Embodiment 185 is the method or composition of embodiment 183, wherein the first genome editing tool, the second genome editing tool, and the gRNAs are collectively contained in 4 distinct LNPs each comprising a distinct nucleic acid component.
   Embodiment 186 is the method or composition of embodiment 183, wherein the first genome editing tool, the second genome editing tool, and the gRNAs are collectively contained in 5 distinct LNPs each comprising a distinct nucleic acid component.
   Embodiment 187 is the method or composition of embodiment 183, wherein the first genome editing tool, the second genome editing tool, and the gRNAs are collectively contained in 6 distinct LNPs each comprising a distinct nucleic acid component.
   Embodiment 188 is the method or composition of embodiment 183, wherein the first genome editing tool, the second genome editing tool, and the gRNAs are collectively contained in 7 distinct LNPs each comprising a distinct nucleic acid component.
   Embodiment 189 is the method or composition of any one of embodiments 1-167, wherein the at least one gRNA that is cognate to the first genomic editor or the base editor and the at least one gRNA that is cognate to the second genomic editor collectively comprise at least 2 gRNAs, and wherein 2 of the gRNAs that target different genomic loci are contained in a same lipid nanoparticle (LNP).
   Embodiment 190 is the method or composition of any one of embodiments 1-167 and 189, wherein the at least one gRNA that is cognate to the first genomic editor or the base editor and the at least one gRNA that is cognate to the second genomic editor collectively comprise at least 3 gRNAs, and wherein 3 of the gRNAs that target different genomic loci are contained in a same lipid nanoparticle.
   Embodiment 191 is the method or composition of any one of embodiments 1-167, 189, and 190, wherein the at least one gRNA that is cognate to the first genomic editor or the base editor and the at least one gRNA that is cognate to the second genomic editor collectively comprise at least 4 gRNAs, and wherein 4 of the gRNAs that target different genomic loci are contained in a same lipid nanoparticle.
   Embodiment 192 is the method or composition of any one of embodiments 189-191, wherein each of the other gRNAs is contained in a different LNP.
   Embodiment 193 is the method or composition of any one of embodiments 1-167, wherein each one of the gRNAs is contained in a different LNP.
   Embodiment 194 is the method or composition of any one of embodiments 1-167, wherein the at least one gRNA that is cognate to the first genomic editor or the base editor comprises more than one gRNAs that target different genomic loci, and the first genomic editor or the base editor is contained in a same LNP with at least one of the more than one gRNAs.
   Embodiment 195 is the method or composition of embodiment 194, wherein the first genomic editor or the base editor and one of the gRNAs are contained in a same LNP.
   Embodiment 196 is the method or composition of embodiment 194 or 195, wherein the first genomic editor or the base editor and 2 of the gRNAs are contained in a same LNP.
   Embodiment 197 is the method or composition of any one of embodiments 194-196, wherein the first genomic editor or the base editor and 3 of the gRNAs are contained in a same LNP.
   Embodiment 198 is the method or composition of any one of embodiments 194-197, wherein the first genomic editor or the base editor and 4 of the gRNAs are contained in a same LNP.
   Embodiment 199 is the method or composition of any one of embodiments 1-167, wherein the first genomic editor or the base editor is contained in a different LNP than each of the at least one gRNA that is cognate to the first genomic editor or the base editor.
   Embodiment 200 is the method or composition of any one of embodiments 1-167, wherein the at least one gRNA that is cognate to the first genomic editor or the base editor comprises more than one gRNAs that target different genomic loci, and each of the more than one gRNAs is contained in a different LNP.
   Embodiment 201 is the method or composition of embodiment 200, wherein each of the LNPs comprising one of the gRNAs cognate to the first genomic editor or the base editor further comprises the first genomic editor or the base editor.
   Embodiment 202 is the method or composition of any one of embodiments 1-167, wherein the second genomic editor and the at least one gRNA that is cognate to the second genomic editor are contained in a same LNP.
   Embodiment 203 is the method or composition of embodiment 202, wherein the second genomic editor is contained in a same LNP with one of the gRNAs.
   Embodiment 204 is the method or composition of any one of embodiments 1-167, wherein the first genome editing tool comprises a uracil glycosylase inhibitor (UGI), and the UGI is contained in a different LNP than each one of the gRNAs.
   Embodiment 205 is the method or composition of any one of embodiments 1-204, wherein the LNPs comprise a first group of distinct LNPs, and a second group of distinct LNPs, and optionally, a third group of distinct LNPs.
   Embodiment 206 is the method or composition of embodiment 205, wherein the first group of distinct LNPs comprises 2, 3, 4, or 5 LNPs, the second group of distinct LNPs comprises 2, 3, 4, or 5 LNPs, and the third group of distinct LNPs, when present, comprises 2, 3, 4, or 5 LNPs.
   Embodiment 207 is the method or composition of embodiment 205 or 206, wherein the first group of distinct LNPs comprises 3 or 4 LNPs, the second group of distinct LNPs comprises 3 or 4 LNPs.
   Embodiment 208 is the method or composition of any one of embodiments 205-207, wherein the first group of distinct LNPs, the second group of distinct LNPs, and the third group of distinct LNPs, when present, are delivered to the cell sequentially.
   Embodiment 209 is the method or composition of any one of embodiments 205-208, wherein the second group of distinct LNPs is delivered to the cell 1, 2, or 3 days after the first group of distinct LNPs is delivered to the cell, and wherein the third group of distinct LNPs, when present, is delivered to the cell 1, 2, or 3 days after the second group of distinct LNPs is delivered to the cell.
   Embodiment 210 is the method or composition of any one of embodiments 205-209, wherein the second group of distinct LNPs is delivered to the cell 1 day after the first group of distinct LNPs is delivered to the cell.
   Embodiment 211 is the method or composition of any one of embodiments 21-86 and 104-210, wherein the LNP has a diameter of 1-250 nm, 10-200 nm, 20-150 nm, about 35-150 nm, 50-150 nm, 50-100 nm, 50-120 nm, 60-100 nm, 75-150 nm, 75-120 nm, or 75-100 nm.
   Embodiment 212 is the method or composition of embodiment 211, wherein the LNP has a diameter of <100 nm.
   Embodiment 213 is the method or composition of any one of embodiments 21-86 and 104-211, wherein the LNP comprises an ionizable lipid.
   Embodiment 214 is the method or composition of embodiment 213, wherein the ionizable lipid comprises a biodegradable ionizable lipid.
   Embodiment 215 is the method or composition of embodiment 213 or 214, wherein the ionizable lipid has a PK value in the range of pKa in the range of from about 5.1 to about 7.4, such as from about 5.5 to about 6.6, from about 5.6 to about 6.4, from about 5.8 to about 6.2, or from about 5.8 to about 6.5.
   Embodiment 216 is the method or composition of any one of embodiments 213-215, wherein the ionizable lipid comprises an amine lipid.
   Embodiment 217 is the method or composition of embodiment 216, wherein the amine lipid is Lipid A or its acetal analog or Lipid D.
   Embodiment 218 is the method or composition of any one of embodiments any one of embodiments 21-86 and 104-217, wherein the LNP comprises a helper lipid.
   Embodiment 219 is the method or composition of any one of embodiments any one of embodiments 21-86 and 104-218, wherein the N/P ratio of the LNP is about 6.
   Embodiment 220 is the method or composition of any one of embodiments any one of embodiments 21-86 and 104-219, wherein the LNP comprises an amine lipid, a helper lipid, and a PEG lipid.
   Embodiment 221 is the method or composition of any one of embodiments any one of embodiments 21-86 and 104-220, wherein the LNP comprises an amine lipid, a helper lipid, a neutral lipid, and a PEG lipid.
   Embodiment 222 is the method or composition of any one of embodiments any one of embodiments 21-86 and 104-221, wherein the LNP comprises a lipid component and the lipid component comprises: about 50-60 mol % amine lipid such as Lipid A; about 8-10 mol % neutral lipid; and about 2.5-4 mol % stealth lipid (e.g., a PEG lipid), wherein the remainder of the lipid component is helper lipid, and wherein the N/P ratio of the lipid LNP is about 3-7.
   Embodiment 223 is the method or composition of any one of embodiments any one of embodiments 21-86 and 104-222, wherein the LNP comprises a lipid component and the lipid component comprises: about 25-45 mol % amine lipid, such as Lipid A; about 10-30 mol % neutral lipid; about 25-65 mol % helper lipid; and about 1.5-3.5 mol % stealth lipid (e.g., PEG lipid), and wherein the N/P ratio of the LNP is about 3-7.
   Embodiment 224 is the method or composition of embodiment 223, wherein the amount of the amine lipid is about 29-38 mol % of the lipid component; about 30-43 mol % of the lipid component; or about 25-34 mol % of the lipid component; optionally about 33 mol %, about 35 mol % of the lipid component, or about 38 mol % of the lipid component.
   Embodiment 225 is the method or composition of 223 or 224, wherein the amount of the neutral lipid is about 11-20 mol % of the lipid component, optionally about 15 mol % of the lipid component.
   Embodiment 226 is the method or composition of any one of embodiments 223-225, wherein the amount of the helper lipid is about 43-65 mol % of the lipid component; or about 43-55 mol % of the lipid component; optionally about 47.5 mol % of the lipid component or about 49 mol % of the lipid component.
   Embodiment 227 is the method or composition of any one of embodiments 223-226, wherein the amount of the PEG lipid is about 2.0-3.5 mol % of the lipid component; about 2.3-3.5 mol % of the lipid component; or about 2.3-2.7 mol % of the lipid component, optionally about 2.5 mol % of the lipid component or about 2.7 mol % of the lipid component.
   Embodiment 228 is the method or composition of any one of embodiments 223-237, wherein
a. the amount of the amine lipid is about 29-44 mol % of the lipid component; the amount of the neutral lipid is about 11-28 mol % of the lipid component; the amount of the helper lipid is about 28-55 mol % of the lipid component; and the amount of the PEG lipid is about 2.3-3.5 mol % of the lipid component
b. the amount of the amine lipid is about 29-38 mol % of the lipid component; the amount of the neutral lipid is about 11-20 mol % of the lipid component; the amount of the helper lipid is about 43-55 mol % of the lipid component; and the amount of the PEG lipid is about 2.3-2.7 mol % of the lipid component;
c. the amount of the amine lipid is about 25-34 mol % of the lipid component; the amount of the neutral lipid is about 10-20 mol % of the lipid component; the amount of the helper lipid is about 45-65 mol % of the lipid component; and the amount of the PEG lipid is about 2.5-3.5 mol % of the lipid component; or
d. the amount of the amine lipid is about 30-43 mol % of the lipid component; the amount of the neutral lipid is about 10-17 mol % of the lipid component; the amount of the helper lipid is about 43.5-56 mol % of the lipid component; and the amount of the PEG lipid is about 1.5-3 mol % of the lipid component.
   Embodiment 229 is the method or composition of any one of embodiments any one of embodiments 21-86 and 104-228, wherein the LNP comprises a lipid component and the lipid component comprises: about 25-50 mol % amine lipid, such as Lipid D; about 7-25 mol % neutral lipid; about 39-65 mol % helper lipid; and about 0.5-1.8 mol % stealth lipid (e.g., PEG lipid), and wherein the N/P ratio of the LNP is about 3-7.
   Embodiment 230 is the method or composition of embodiment 229, wherein the amount of the amine lipid is about 30-45 mol % of the lipid component; or about 30-40 mol % of the lipid component; optionally about 30 mol %, 40 mol %, or 50 mol % of the lipid component.
   Embodiment 231 is the method or composition of embodiment 229 or 230, wherein the amount of the neutral lipid is about 10-20 mol % of the lipid component; or about 10-15 mol % of the lipid component; optionally about 10 mol % or 15 mol % of the lipid component.
   Embodiment 232 is the method or composition of any one of embodiments 229-231, wherein the amount of the helper lipid is about 50-60 mol % of the lipid component; about 39-59 mol % of the lipid component; or about 43.5-59 mol % of the lipid component; optionally about 59 mol % of the lipid component; about 43.5 mol % of the lipid component; or about 39 mol % of the lipid component.
   Embodiment 233 is the method or composition of any one of embodiments 229-232, wherein the amount of the PEG lipid is about 0.9-1.6 mol % of the lipid component; or about 1-1.5 mol % of the lipid component; optionally about 1 mol % of the lipid component or about 1.5 mol % of the lipid component.
   Embodiment 234 is the method or composition of any one of embodiments 229-233, wherein:
a. the amount of the ionizable lipid is about 27-40 mol % of the lipid component; the amount of the neutral lipid is about 10-20 mol % of the lipid component; the amount of the helper lipid is about 50-60 mol % of the lipid component; and the amount of the PEG lipid is about 0.9-1.6 mol % of the lipid component;
b. the amount of the ionizable lipid is from about 30-45 mol % of the lipid component; the amount of the neutral lipid is from about 10-15 mol % of the lipid component; the amount of the helper lipid is from about 39-59 mol % of the lipid component; and the amount of the PEG lipid is from about 1-1.5 mol % of the lipid component;
c. the amount of the ionizable lipid is about 30 mol % of the lipid component; the amount of the neutral lipid is about 10 mol % of the lipid component; the amount of the helper lipid is about 59 mol % of the lipid component; and the amount of the PEG lipid is about 1 mol % of the lipid component;
d. the amount of the ionizable lipid is about 40 mol % of the lipid component; the amount of the neutral lipid is about 15 mol % of the lipid component; the amount of the helper lipid is about 43.5 mol % of the lipid component; and the amount of the PEG lipid is about 1.5 mol % of the lipid component; or
e. the amount of the ionizable lipid is about 50 mol % of the lipid component; the amount of the neutral lipid is about 10 mol % of the lipid component; the amount of the helper lipid is about 39 mol % of the lipid component; and the amount of the PEG lipid is about 1 mol % of the lipid component.
   Embodiment 235 is the method or composition of any one of embodiments 216-234, wherein the amine lipid is Lipid A.
   Embodiment 236 is the method or composition of any one of embodiments 216-234, wherein the amine lipid is Lipid D.
   Embodiment 237 is the method or composition of any one of embodiments 221-236, wherein the neutral lipid is DSPC.
   Embodiment 238 is the method or composition of any one of embodiments 222-237, wherein the stealth lipid is PEG-dimyristoylglycerol (PEG-DMG).
   Embodiment 239 is the method or composition of any one of embodiments 218-238, wherein the helper lipid is cholesterol.
   Embodiment 240 is the method or composition of any one of embodiments 21-86 and 104-239, wherein the LNP is pretreated with a serum factor before contacting the cell, optionally wherein the serum factor is a primate serum factor, optionally a human serum factor.
   Embodiment 241 is the method or composition of any one of embodiments 21-86 and 104-240, wherein the LNP is pretreated with a human serum before contacting the cell.
   Embodiment 242 is the method or composition of any one of embodiments 21-86 and 104-241, wherein the LNP is pretreated with an ApoE before contacting the cell, optionally wherein the ApoE is a human ApoE.
   Embodiment 243 is the method or composition of any one of embodiments 21-86 and 104-242, wherein the LNP is pretreated with a recombinant ApoE3 or ApoE4 before contacting the cell, optionally wherein the ApoE3 or ApoE4 is a human ApoE3 or ApoE4.
   Embodiment 244 is a cell, wherein the cell is treated in vitro with the method or composition of any one of embodiments 1-243.
   Embodiment 245 is a cell, wherein the cell is treated in vivo with the method or composition of any one of embodiments 1-243.
   Embodiment 246 is the cell of embodiment 244 or 245, wherein the cell is a human cell.
   Embodiment 247 is the cell of any one of embodiments 244-246, wherein the cell is selected from: a mesenchymal stem cell; a hematopoietic stem cell (HSC); a mononuclear cell; an endothelial progenitor cells (EPC); a neural stem cells (NSC); a limbal stem cell (LSC); a tissue-specific primary cell or a cell derived therefrom (TSC), an induced pluripotent stem cell (iPSC); an ocular stem cell; a pluripotent stem cell (PSC); an embryonic stem cell (ESC); and a cell for organ or tissue transplantation, and optionally a cell for use in ACT therapy.
   Embodiment 248 is the cell of any one of embodiments 244-247, wherein the cell is an immune cell.
   Embodiment 249 is the cell of embodiment 248, wherein the immune cell is selected from a lymphocyte (e.g., T cell, B cell, natural killer cell (“NK cell”, and NKT cell, or iNKT cell)), a monocyte, a macrophage, a mast cell, a dendritic cell, a granulocyte (e.g., neutrophil, eosinophil, and basophil), a primary immune cell, a CD3+ cell, a CD4+ cell, a CD8+ T cell, a regulatory T cell (Treg), a B cell, and a dendritic cell (DC)).
   Embodiment 250 is the cell of embodiment 248, wherein the immune cell is selected from a peripheral blood mononuclear cell (PBMC), a lymphocyte, a T cell, optionally a CD4+ cell, a CD8+ cell, a memory T cell, a naïve T cell, a stem-cell memory T cell; or a B cell, optionally a memory B cell, a naïve B cell; and a primary cell.
   Embodiment 251 is the cell of embodiment 250, wherein the cell is a T cell.
   Embodiment 252 is the cell of embodiment 251, wherein the T cell is selected from a tumor infiltrating lymphocyte (TIL), a T cell expressing an alpha-beta TCR, a T cell expressing a gamma-delta TCR, a regulatory T cell (Treg), a memory T cell, and an early stem cell memory T cell (Tscm, CD27+/CD45+).
   Embodiment 253 is the cell of any one of embodiments 244-252, wherein the cell is isolated from human donor PBMCs or leukopaks before editing.
   Embodiment 254 is the cell of any one of embodiments 244-253, wherein the cell is derived from a progenitor cell before editing.
   Embodiment 255 is a population of cells, comprising the cell of any one of embodiments 244-254.
   Embodiment 256 is the population of cells of embodiment 255, wherein the population comprises edited T cells, and wherein at least 30%, 40%, 50%, 55%, 60%, 65% of the cells of the population have a memory phenotype (CD27+, CD45RA+).
   Embodiment 257 is the population of cells of embodiment 255 or 256, wherein the cells are non-activated immune cells.
   Embodiment 258 is the population of cells of any one of embodiments 255-257, wherein the cells are activated immune cells.
   Embodiment 259 is the population of cells of any one of embodiments 255-258, wherein the cells are T cells and the cells are responsive to repeat stimulation after editing.
   Embodiment 260 is the population of cells of any one of embodiments 255-259, wherein the cells are cultured, expanded, or proliferated ex vivo.
   Embodiment 261 is the cell, the population of cells, or the composition of any one of embodiments 87-260, for use in treating cancer.
   Embodiment 262 Use of the cell, the population of cells, or the composition of any one of embodiments 87-261 for preparation of a medicament for treating cancer.
   Embodiment 263 is an engineered cell comprising at least three base edits in at least three genomic loci, and at least one exogenous gene.
   Embodiment 264 is a composition comprising:
a. a gRNA comprising a guide sequence chosen from: i) SEQ ID NOs: 251-264; ii) at least 17, 18, 19, or 20 contiguous nucleotides of a sequence selected from SEQ ID NOs: 251-264; iii) a guide sequence at least 95%, 90%, or 85% identical to a sequence selected from SEQ ID NOs: 251-264; iv) a sequence that comprises 10 contiguous nucleotides±10 nucleotides of a genomic coordinate listed in Table 5; v) at least 17, 18, 19, or 20 contiguous nucleotides of a sequence from (iv); or vi) a guide sequence that is at least 95%, 90%, or 85% identical to a sequence selected from (v); or
b. a nucleic acid encoding a gRNA of (a.).
   Embodiment 265 is a method of altering a DNA sequence within an AAVS1 gene, comprising delivering to a cell:
a. a gRNA comprising a guide sequence chosen from: i) SEQ ID NOs: 251-264; ii) at least 17, 18, 19, or 20 contiguous nucleotides of a sequence selected from SEQ ID NOs: 251-264; iii) a guide sequence at least 95%, 90%, or 85% identical to a sequence selected from SEQ ID NOs: 251-264; iv) a sequence that comprises 10 contiguous nucleotides±10 nucleotides of a genomic coordinate listed in Table 5; v) at least 17, 18, 19, or 20 contiguous nucleotides of a sequence from (iv); or vi) a guide sequence that is at least 95%, 90%, or 85% identical to a sequence selected from (v); or
b. a nucleic acid encoding a gRNA of (a.).
   Embodiment 266 is a method of immunotherapy comprising administering a composition comprising an engineered cell to a subject,
wherein the cell comprises a genomic modification in the AAVS1 gene, wherein the genetic modification comprises an insertion within the genomic coordinates selected from: chr19:55115695-55115715; chr19:55115588-55115608; chr19:55115616-55115636; chr19:55115623-55115643; chr19:55115637-55115657; chr19:55115691-55115711; chr19:55115755-55115775; chr19:55115823-55115843; chr19:55115834-55115854; chr19:55115835-55115855; chr19:55115836-55115856; chr19:55115850-55115870; chr19:55115951-55115971; and chr19:55115949-55115969; or
wherein the cell is engineered by delivering to the cell:
a. a gRNA comprising a guide sequence chosen from: i) SEQ ID NOs: 251-264; ii) at least 17, 18, 19, or 20 contiguous nucleotides of a sequence selected from SEQ ID NOs: 251-264; iii) a guide sequence at least 95%, 90%, or 85% identical to a sequence selected from SEQ ID NOs: 251-264; iv) a sequence that comprises 10 contiguous nucleotides±10 nucleotides of a genomic coordinate listed in Table 5; v) at least 17, 18, 19, or 20 contiguous nucleotides of a sequence from (iv); or vi) a guide sequence that is at least 95%, 90%, or 85% identical to a sequence selected from (v); or
b. a nucleic acid encoding a gRNA of (a.).
   Embodiment 267 is an engineered cell comprising a genetic modification in the AAVS1 gene, wherein the genetic modification comprises an insertion within the genomic coordinates chosen from: chr19:55115695-55115715; chr19:55115588-55115608; chr19:55115616-55115636; chr19:55115623-55115643; chr19:55115637-55115657; chr19:55115691-55115711; chr19:55115755-55115775; chr19:55115823-55115843; chr19:55115834-55115854; chr19:55115835-55115855; chr19:55115836-55115856; chr19:55115850-55115870; chr19:55115951-55115971; and chr19:55115949-55115969.
   Embodiment 268 is a method or composition of any one of embodiments 1, 16, 17, 87, and 101, wherein the first genome editing tool, the second genome editing tool, and the gRNAs are collectively contained in:
(a)
   (i) a first lipid nanoparticle (LNP) comprising a uracil glycosylase inhibitor (UGI); (ii) a second LNP comprising the first genomic editor or the base editor and comprising a second gRNA; (iii) a third LNP comprising the first genomic editor or the base editor and comprising a third gRNA; and (iv) a fourth LNP comprising the first genomic editor or the base editor and comprising a fourth gRNA; and
(b)
   (i) a fifth LNP comprising a uracil glycosylase inhibitor (UGI); (ii) a sixth LNP comprising the second genomic editor and a first gRNA; (iii) a nucleic acid encoding an exogenous gene for insertion at an editing site of the first gRNA; (iv) optionally an seventh LNP comprising the first genomic editor or the base editor and comprising a fifth gRNA; (v) optionally a eighth LNP comprising the first genomic editor or the base editor and comprising a sixth gRNA; (vi) optionally a ninth LNP comprising the first genomic editor or the base editor and comprising a seventh gRNA.

### I. DEFINITIONS

Unless stated otherwise, the following terms and phrases as used herein are intended to have the following meanings:

“Polynucleotide” and “nucleic acid” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy, 2′ halide, or 2′-O-(2-methoxyethyl) (2′-O-moe) substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). Nucleic acid includes “unlocked nucleic acid” enables the modulation of the thermodynamic stability and also provides nuclease stability. RNA and DNA have different sugar moieties and can differ by the presence of uracil or analogs thereof in RNA and thymine or analogs thereof in DNA.

“Polypeptide” as used herein refers to a multimeric compound comprising amino acid residues that can adopt a three-dimensional conformation. Polypeptides include but are not limited to enzymes, enzyme precursor proteins, regulatory proteins, structural proteins, receptors, nucleic acid binding proteins, antibodies, etc. Polypeptides may, but do not necessarily, comprise post-translational modifications, non-natural amino acids, prosthetic groups, and the like.

As used herein, “ribonucleoprotein” (RNP) or “RNP complex” refers to a guide RNA together with an RNA-guided DNA binding agent, such as a Cas nuclease, e.g., a Cas cleavase, Cas nickase, or dCas DNA binding agent (e.g., Cas9). In some embodiments, the guide RNA guides the RNA-guided DNA binding agent such as Cas9 to a target sequence, and the guide RNA hybridizes with the target sequence and the agent binds to the target sequence; in cases where the agent is a cleavase or nickase, binding can be followed by cleaving or nicking.

As used herein, an “RNA-guided DNA binding agent” means a polypeptide or complex of polypeptides having RNA and DNA binding activity, or a DNA-binding subunit of such a complex, wherein the DNA binding activity is sequence-specific and depends on the presence of a PAM and the sequence of the guide RNA. Exemplary RNA-guided DNA binding agents include Cas cleavases/nickases and inactivated forms thereof (“dCas DNA binding agents”). “Cas nuclease”, also called “Cas protein” as used herein, encompasses Cas cleavases, Cas nickases, and dCas DNA binding agents. Cas cleavases/nickases and dCas DNA binding agents include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. As used herein, a “Class 2 Cas nuclease” is a single-chain polypeptide with RNA-guided DNA binding activity. Class 2 Cas nucleases include Class 2 Cas cleavases/nickases (e.g., H840A, D10A, or N863A variants), which further have RNA-guided DNA cleavases or nickase activity, and Class 2 dCas DNA binding agents, in which cleavase/nickase activity is inactivated. Class 2 Cas nucleases include, for example, Cas9, Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9 (1.0) (e.g., K810A, K1003A, R1060A variants), and eSPCas9 (1.1) (e.g., K848A, K1003A, R1060A variants) proteins and modifications thereof. Cpf1 protein, Zetsche et al., Cell, 163: 1-13 (2015), is homologous to Cas9, and contains a RuvC-like nuclease domain. Cpf1 sequences of Zetsche are incorporated by reference in their entirety. See, e.g., Zetsche, Tables S1 and S3. See, e.g., Makarova et al., Nat Rev Microbiol, 13(11): 722-36 (2015); Shmakov et al., Molecular Cell, 60:385-397 (2015).

As used herein, the term “genomic editor” or “editor” refers to an agent comprising a polypeptide that is capable of making a modification within a nucleic acid sequence (e.g., DNA or RNA). In some embodiments, the editor is a cleavase, such as a Cas9 cleavase. In some embodiments, the editor is capable of deaminating a base within a nucleic acid, and it may be called a base editor. In some embodiments, the editor is capable of deaminating a base within a DNA molecule. In some embodiments, the editor is capable of deaminating a cytosine (C) in DNA. In some embodiments, the editor is a fusion protein comprising an RNA-guided nickase fused to a cytidine deaminase domain. In some embodiments, the editor is a combination of an RNA-guided nickase and a cytidine deaminase domain. In some embodiments, the editor is a fusion protein comprising an RNA-guided nickase fused to an APOBEC3A deaminase (A3A). In some embodiments, the editor comprises a Cas9 nickase fused to an APOBEC3A deaminase (A3A). In some embodiments, the editor is a fusion protein comprising an enzymatically inactive RNA-guided DNA-binding protein fused to a cytidine deaminase domain. In some embodiments, the editor is a nickase fused to a DNA polymerase.

As used herein, the term “genome editing tool” refers to an agent comprising a genomic editor and at least one guide RNA cognate to a nuclease or nickase component of the genomic editor.

A genomic editor, for example, may comprise a C to T base editor, and may or may not comprise a uracil glycosylase inhibitor (UGI). A genomic editor, for example, may comprise a cytidine deaminase, an RNA-guided nickase, and a UGI, wherein the cytidine deaminase, the RNA-guided nickase, and the UGI are comprised in a single polypeptide, wherein the cytidine deaminase, the RNA-guided nickase, and the UGI are comprised in different polypeptides, or wherein the deaminase and the RNA-guided nickase are comprised in a single polypeptide, and the UGI is comprised in a different polypeptide. In some embodiments, the deaminase comprises a cytidine deaminase.

As used herein, the term “orthogonal” refers to any two genomic editors (e.g., base editors, nucleases, nickases, or cleavases) where each is capable of recognizing its own target(s) via its cognate guide RNA(s) but not compatible with the guide RNA(s) cognate to the other genomic editor, e.g., each is not capable of recognizing the target(s) of the other genomic editor via the guide RNA(s) cognate to the other genomic editor. For example, an N. meningitidis Cas9 (NmeCas9) nickase may be capable of recognizing a genomic locus via a guide RNA cognate to the NmeCas9 nickase, and an S. pyogenes Cas9 (SpyCas9) cleavase may be capable of recognizing another genomic locus via a guide RNA cognate to the SpyCas9 cleavase. In this example, the NmeCas9 nickase and the SpyCas9 cleavase are orthogonal to each other. Genome editors or genome editing components may be engineered to be orthogonal. Although in this example, the NmeCas9 nickase and the SpyCas9 cleavase are derived from different organisms, two genomic editors need not be derived from different organisms to be orthogonal to each other.

As used herein, a “cytidine deaminase” means a polypeptide or complex of polypeptides that is capable of cytidine deaminase activity, that is catalyzing the hydrolytic deamination of cytidine or deoxycytidine, typically resulting in uridine or deoxyuridine. Cytidine deaminases encompass enzymes in the cytidine deaminase superfamily, and in particular, enzymes of the APOBEC family (APOBEC1, APOBEC2, APOBEC4, and APOBEC3 subgroups of enzymes), activation-induced cytidine deaminase (AID or AICDA) and CMP deaminases (see, e.g., Conticello et al., Mol. Biol. Evol. 22:367-77, 2005; Conticello, Genome Biol. 9:229, 2008; Muramatsu et al., J. Biol. Chem. 274: 18470-6, 1999); Carrington et al., Cells 9:1690 (2020)). In some embodiments, variants of any known cytidine deaminase or APOBEC protein are encompassed. Variants include proteins having a sequence that differs from wild-type protein by one or several mutations (i.e., substitutions, deletions, insertions), such as one or several single point substitutions. For instance, a shortened sequence could be used, e.g., by deleting N-terminal, C-terminal, or internal amino acids, preferably one to four amino acids at the C-terminus of the sequence. As used herein, the term “variant” refers to allelic variants, splicing variants, and natural or artificial mutants, which are homologous to a reference sequence. The variant is “functional” in that it shows a catalytic activity of DNA editing.

As used herein, the term “APOBEC3A” refers to a cytidine deaminase such as the protein expressed by the human A3A gene. The APOBEC3A may have catalytic DNA editing activity. An amino acid sequence of APOBEC3A has been described (UniPROT accession ID: p31941) and is included herein as SEQ ID NO: 22. In some embodiments, the APOBEC3A protein is a human APOBEC3A protein or a wild-type protein. Variants include proteins having a sequence that differs from wild-type APOBEC3A protein by one or several mutations (i.e., substitutions, deletions, insertions), such as one or several single point substitutions. For instance, a shortened APOBEC3A sequence could be used, e.g. by deleting N-terminal, C-terminal, or internal amino acids, preferably one to four amino acids at the C-terminus of the sequence. As used herein, the term “variant” refers to allelic variants, splicing variants, and natural or artificial mutants, which are homologous to an APOBEC3A reference sequence. The variant is “functional” in that it shows a catalytic activity of DNA editing. In some embodiments, an APOBEC3A (such as a human APOBEC3A) has a wild-type amino acid position 57 (as numbered in the wild-type sequence). In some embodiments, an APOBEC3A (such as a human APOBEC3A) has an asparagine at amino acid position 57 (as numbered in the wild-type sequence).

As used herein, a “nickase” is an enzyme that creates a single-strand break (also known as a “nick”) in double strand DNA, i.e., cuts one strand but not the other of the DNA double helix. As used herein, an “RNA-guided nickase” means a polypeptide or complex of polypeptides having DNA nickase activity, wherein the DNA nickase activity is sequence-specific and depends on the sequence of the RNA. Exemplary RNA-guided nickases include Cas nickases. Cas nickases include, but are not limited to, nickase forms of a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. Class 2 Cas nickases include, polypeptides in which either the HNH or RuvC catalytic domain is inactivated, for example, Cas9 (e.g., H840A, D10A, or N863A variants of SpyCas9 or D16A variant of NmeCas9). Exemplary amino acid substitutions in the HNH or HNH-like nuclease domain or RuvC or RuvC-like domains for N. meningitidis include Nme2Cas9D16A (HNH nickase) and Nme2Cas9H588A (RuvC nickase). Class 2 Cas nickases include, for example, Cas9 (e.g., H840A, D10A, or N863A variants of SpyCas9), Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9 (1.0) (e.g., K810A, K1003A, R1060A variants), and eSPCas9 (1.1) (e.g., K848A, K1003A, R1060A variants) proteins and modifications thereof. Cpf1 protein, Zetsche et al., Cell, 163: 1-13 (2015), is homologous to Cas9, and contains a RuvC-like protein domain. Cpf1 sequences of Zetsche are incorporated by reference in their entirety. See, e.g., Zetsche, Tables S1 and S3. “Cas9” encompasses S. pyogenes (Spy) Cas9, the variants of Cas9 listed herein, and equivalents thereof. See, e.g., Makarova et al., Nat Rev Microbiol, 13(11): 722-36 (2015); Shmakov et al., Molecular Cell, 60:385-397 (2015).

As used herein, the term “fusion protein” refers to a hybrid polypeptide which comprises polypeptides from at least two different proteins or sources. One polypeptide may be located at the amino-terminal (N-terminal) portion of the fusion protein or at the carboxy-terminal (C-terminal) protein thus forming an “amino-terminal fusion protein” or a “carboxy-terminal fusion protein,” respectively. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

The term “linker,” as used herein, refers to a chemical group or a molecule linking two adjacent molecules or moieties. Typically, the linker is positioned between, or flanked by, two groups, molecules, or other moieties and connected to each one via a covalent bond. In some embodiments, the linker is an amino acid or a plurality of amino acids (e.g., a peptide or protein) such as a 16-amino acid residue “XTEN” linker, or a variant thereof (See, e.g., the Examples; and Schellenberger et al. A recombinant polypeptide extends the in vivo half-life of peptides and proteins in a tunable manner. Nat. Biotechnol. 27, 1186-1190 (2009)). In some embodiments, the XTEN linker comprises the sequence SGSETPGTSESATPES (SEQ ID NO: 25), SGSETPGTSESA (SEQ ID NO: 26), or SGSETPGTSESATPEGGSGGS (SEQ ID NO: 27). In some embodiments, the linker comprises one or more sequences selected from SEQ ID NOs: 25-39 and 72-133.

As used herein, the term “uracil glycosylase inhibitor”, “uracil-DNA glycosylase inhibitor” or “UGI” refers to a protein that is capable of inhibiting a uracil-DNA glycosylase (UDG) base-excision repair enzyme (e.g., UniPROT ID: P14739; SEQ ID NO: 15; SEQ ID NO: 24).

As used herein, the terms “nuclear localization signal” (NLS) or “nuclear localization sequence” refers to an amino acid sequence which induces transport of molecules comprising such sequences or linked to such sequences into the nucleus of eukaryotic cells. The nuclear localization signal may form part of the molecule to be transported. In some embodiments, the NLS may be fused to the molecule by a covalent bond, hydrogen bonds or ionic interactions. In some embodiments, the NLS may be fused to the molecule via a linker.

As used herein, “open reading frame” or “ORF” of a gene refers to a sequence consisting of a series of codons that specify the amino acid sequence of the protein that the gene codes for. The ORF generally begins with a start codon (e.g., ATG in DNA or AUG in RNA) and ends with a stop codon, e.g., TAA, TAG or TGA in DNA or UAA, UAG, or UGA in RNA.

“Guide RNA”, “gRNA”, and “guide” are used herein interchangeably to refer to either a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” or “gRNA” refers to each type. The trRNA may be a naturally-occurring sequence, or a trRNA sequence with modifications or variations compared to naturally-occurring sequences.

As used herein, a “guide sequence” or “guide region” or “targeting sequence” or “spacer” or “spacer sequence” and the like refers to a sequence within a gRNA that is complementary to a target sequence and functions to direct a gRNA to a target sequence for binding or modification (e.g., cleavage) by an RNA-guided nickase. A guide sequence can be 20 nucleotides in length, e.g., in the case of Streptococcus pyogenes (i.e., Spy Cas9 (also referred to as SpCas9)) and related Cas9 homologs/orthologs. Shorter or longer sequences can also be used as guides, e.g., 15-, 16-, 17-, 18-, 19-, 21-, 22-, 23-, 24-, or 25-nucleotides in length. A guide sequence can be 20-25 nucleotides in length, e.g., in the case of Nine Cas9, e.g., 20-, 21-, 22-, 23-, 24- or 25-nucleotides in length. For example, a guide sequence of 24 nucleotides in length can be used with Nine Cas9, e.g., Nme2 Cas9.

In some embodiments, the target sequence is in a genomic locus or on a chromosome, for example, and is complementary to the guide sequence. In some embodiments, the degree of complementarity or identity between a guide sequence and its corresponding target sequence may be about 75%, 80%, 85%, 90%, 95%, or 100%. In some embodiments, the guide sequence and the target region may be 100% complementary or identical. In other embodiments, the guide sequence and the target region may contain at least one mismatch. For example, the guide sequence and the target sequence may contain 1, 2, 3, or 4 mismatches, where the total length of the target sequence is at least 17, 18, 19, 20 or more base pairs. In some embodiments, the guide sequence and the target region may contain 1-4 mismatches where the guide sequence comprises at least 17, 18, 19, 20 or more nucleotides. In some embodiments, the guide sequence and the target region may contain 1, 2, 3, or 4 mismatches where the guide sequence comprises 20 nucleotides. In some embodiments, the degree of complementarity or identity between a guide sequence and its corresponding target sequence is at least 80%, 85%, 90%, or 95%, for example when, the guide sequence comprises a sequence 24 contiguous nucleotides. In some embodiments, the guide sequence and the target region may be 100% complementary or identical. In other embodiments, the guide sequence and the target region may contain at least one mismatch, i.e., one nucleotide that is not identical or not complementary, depending on the reference sequence. For example, the guide sequence and the target sequence may contain 1-2, preferably no more than 1 mismatch, where the total length of the target sequence is 19, 20, 21, 22, 23, or 24, nucleotides, or more. In some embodiments, the guide sequence and the target region may contain 1-2 mismatches where the guide sequence comprises at least 24 nucleotides, or more. In some embodiments, the guide sequence and the target region may contain 1-2 mismatches where the guide sequence comprises 24 nucleotides.

As used herein, a “target sequence” or “genomic target sequence” refers to a sequence of nucleic acid in a target genomic locus, in either the positive or the negative strand, that has complementarity to the guide sequence of the gRNA, i.e., that is sufficiently complementary to the guide sequence of the gRNA to permit specific binding of the guide to the target sequence. The interaction of the target sequence and the guide sequence directs an RNA-guided DNA binding agent to bind, and potentially nick or cleave (depending on the activity of the agent), within the target sequence. The specific length of the target sequence and the number of mismatches possible between the target sequence and the guide sequence depend, for example, on the identity of the Cas9 nuclease being directed by the gRNA. Target sequences for Cas proteins include both the positive and negative strands of genomic DNA (i.e., the sequence given and the sequence's reverse complement), as a nucleic acid substrate for a Cas protein is a double stranded nucleic acid. Accordingly, where a guide sequence is said to be “complementary to a target sequence,” it is to be understood that the guide sequence may direct an RNA-guided DNA binding agent (e.g., dCas9 or impaired Cas9) to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server are generally appropriate.

“mRNA” is used herein to refer to a polynucleotide that is not DNA and comprises an open reading frame that can be translated into a polypeptide (i.e., can serve as a substrate for translation by a ribosome and amino-acylated tRNAs). mRNA can comprise one or more modifications, e.g. as provided below. In general, mRNAs do not contain a substantial quantity of thymidine residues (e.g., 0 residues or fewer than 30, 20, 10, 5, 4, 3, or 2 thymidine residues; or less than 10%, 9%, 8%, 7%, 6%, 5%, 4%, 4%, 3%, 2%, 1%, 0.5%, 0.2%, or 0.1% thymidine content). An mRNA can contain modified uridines at some or all of its uridine positions.

“Modified uridine” is used herein to refer to a nucleoside other than thymidine with the same hydrogen bond acceptors as uridine and one or more structural differences from uridine. In some embodiments, a modified uridine is a substituted uridine, i.e., a uridine in which one or more non-proton substituents (e.g., alkoxy, such as methoxy) takes the place of a proton. In some embodiments, a modified uridine is pseudouridine. In some embodiments, a modified uridine is a substituted pseudouridine, i.e., a pseudouridine in which one or more non-proton substituents (e.g., alkyl, such as methyl) takes the place of a proton. In some embodiments, a modified uridine is any of a substituted uridine, pseudouridine, or a substituted pseudouridine.

“Uridine position” as used herein refers to a position in a polynucleotide occupied by a uridine or a modified uridine. Thus, for example, a polynucleotide in which “100% of the uridine positions are modified uridines” contains a modified uridine at every position that would be a uridine in a conventional RNA (where all bases are standard A, U, C, or G bases) of the same sequence. Unless otherwise indicated, a U in a polynucleotide sequence of a sequence table or sequence listing in or accompanying this disclosure can be a uridine or a modified uridine.

As used herein, the “minimal uridine codon(s)” for a given amino acid is the codon(s) with the fewest uridines (usually 0 or 1 except for a codon for phenylalanine, where the minimal uridine codon has 2 uridines). Modified uridine residues are considered equivalent to uridines for the purpose of evaluating uridine content.

As used herein, the “uridine dinucleotide (UU) content” of an ORF can be expressed in absolute terms as the enumeration of UU dinucleotides in an ORF or on a rate basis as the percentage of positions occupied by the uridines of uridine dinucleotides (for example, AUUAU would have a uridine dinucleotide content of 40% because 2 of 5 positions are occupied by the uridines of a uridine dinucleotide). Modified uridine residues are considered equivalent to uridines for the purpose of evaluating uridine dinucleotide content.

As used herein, the “minimal adenine codon(s)” for a given amino acid is the codon(s) with the fewest adenines (usually 0 or 1 except for a codon for lysine and asparagine, where the minimal adenine codon has 2 adenines). Modified adenine residues are considered equivalent to adenines for the purpose of evaluating adenine content.

As used herein, the “adenine dinucleotide content” of an ORF can be expressed in absolute terms as the enumeration of AA dinucleotides in an ORF or on a rate basis as the percentage of positions occupied by the adenines of adenine dinucleotides (for example, UAAUA would have an adenine dinucleotide content of 40% because 2 of 5 positions are occupied by the adenines of an adenine dinucleotide). Modified adenine residues are considered equivalent to adenines for the purpose of evaluating adenine dinucleotide content.

As used herein, the term “genomic locus,” when used in the context of a genomic locus being targeted by a guide RNA, includes one or more parts of a genome, the targeting of which affects the expression of the gene that is associated with the locus. For example, a genomic locus may include a coding sequence of a gene, an intron sequence of a gene, a regulatory sequence, a transcriptional control sequence of a gene, a translational control sequence of a gene, a splicing site, or a non-coding sequence between genes (e.g., intergenic space).

As used herein, the term “contact” refers to providing at least one component so that the component physically contacts a cell, including physically contacting the cell surface, cytosol, and/or nucleus of the cell. “Contacting” a cell with a polypeptide encompasses, for example, contacting the cell with a nucleic acid that encodes the polypeptide and allowing the cell to express the polypeptide.

As used herein, the term “simultaneous,” when used in the context of contacting a cell with at least two genome editing tools (e.g., compositions, polypeptides, nucleic acids, or combinations thereof), refers to the contacting of the cell with one of the at least two genome editing tools being no more than 48 hours from the contacting of the cell with the other of the at least two genome editing tools. In some embodiments, the contacting of the cell with one of the at least two genome editing tools is no more than 36 hours from the contacting of the cell with the other of the at least two genome editing tools. In some embodiments, the contacting of the cell with one of the at least two genome editing tools is no more than 24 hours from the contacting of the cell with the other of the at least two genome editing tools. In some embodiments, the contacting of the cell with one of the at least two genome editing tools is no more than 18 hours from the contacting of the cell with the other of the at least two genome editing tools. In some embodiments, the contacting of the cell with one of the at least two genome editing tools is no more than 12 hours from the contacting of the cell with the other of the at least two genome editing tools. In some embodiments, the contacting of the cell with one of the at least two genome editing tools is no more than 6 hours from the contacting of the cell with the other of the at least two genome editing tools. In some embodiments, the contacting of the cell with one of the at least two genome editing tools is no more than 4 hours from the contacting of the cell with the other of the at least two genome editing tools. In some embodiments, the contacting of the cell with one of the at least two genome editing tools is no more than 3 hours from the contacting of the cell with the other of the at least two genome editing tools. In some embodiments, the contacting of the cell with one of the at least two genome editing tools is no more than 2 hours from the contacting of the cell with the other of the at least two genome editing tools. In some embodiments, the contacting of the cell with one of the at least two genome editing tools is no more than 1 hour from the contacting of the cell with the other of the at least two genome editing tools. In some embodiments, the contacting of the cell with one of the at least two genome editing tools is no more than 30 minutes from the contacting of the cell with the other of the at least two genome editing tools. In some embodiments, the contacting of the cell with one of the at least two genome editing tools is no more than 15 minutes from the contacting of the cell with the other of the at least two genome editing tools. In some embodiments, the contacting of the cell with one of the at least two genome editing tools is no more than 10 minutes from the contacting of the cell with the other of the at least two genome editing tools. In some embodiments, the contacting of the cell with one of the at least two genome editing tools is no more than 5 minutes from the contacting of the cell with the other of the at least two genome editing tools. In some embodiments, the contacting of the cell with one of the at least two genome editing tools is at the same time as the contacting of the cell with the other of the at least two genome editing tools. In some embodiments, the two genome editing tools are premixed prior to contacting the cell.

As used herein, “indel” refers to an insertion or deletion mutation consisting of a number of nucleotides that are either inserted, deleted, or inserted and deleted, e.g., at the site of double-stranded breaks (DSBs), in a target nucleic acid. As used herein, when indel formation results in an insertion, the insertion is a random insertion at the site of a DSB and is not generally directed by or based on a template sequence.

As used herein, “knockdown” refers to a decrease in expression of a particular gene product (e.g., protein, mRNA, or both). Knockdown of a protein can be measured either by detecting protein secreted by tissue or population of cells (e.g., in serum or cell media) or by detecting total cellular amount of the protein from a tissue or cell population of interest. Methods for measuring knockdown of mRNA are known and include sequencing of mRNA isolated from a tissue or cell population of interest. In some embodiments, “knockdown” may refer to some loss of expression of a particular gene product, for example a decrease in the amount of mRNA transcribed or a decrease in the amount of protein expressed or secreted by a population of cells (including in vivo populations such as those found in tissues).

As used herein, “knockout” refers to a loss of expression of a particular protein in a cell. Knockout can be measured either by detecting the amount of protein secretion from a tissue or population of cells (e.g., in serum or cell media) or by detecting total cellular amount of a protein a tissue or a population of cells. In some embodiments, the methods of the disclosure “knockout” a target protein one or more cells (e.g., in a population of cells including in vivo populations such as those found in tissues). In some embodiments, a knockout is not the formation of mutant of the target protein, for example, created by indels, but rather the complete loss of expression of the target protein in a cell, i.e., decrease of expression to below the level of detection of the assay used.

As used herein, a “cell population comprising edited cells,” or a “population of cells comprising edited cells,” or the like refers to a cell population that comprises edited cells, however not all cells in the population must be edited. A cell population comprising edited cells may also include non-edited cells. The percentage of edited cells within a cell population comprising edited cells may be determined by counting the number of cells within the population that are edited in the population as determined by standard cell counting methods. For example, in some embodiments, a cell population comprising edited cells comprising a single genome edit will have at least 20%, 30%, 40%, preferably at least 50%, 60%, 70%, 80%, 90%, 95%, or 99% of the cells in the population with the single edit. In some embodiments, a cell population comprising edited cells comprising at least two genome edits will have at least 20%, 30%, 40%, 50%, 60%, 70%, 80%, 90%, or 95% of the cells in the population with at least two genome edits.

“β2M” or “B2M,” as used herein, refers to nucleic acid sequence or protein sequence of “β-2 microglobulin;” the human gene has accession number NC_000015 (range 44711492 . . . 44718877), reference GRCh38.p13. The B2M protein is associated with MHC class I molecules as a heterodimer on the surface of nucleated cells and is required for MHC class I protein expression.

“CIITA” or “CIITA” or “C2TA,” as used herein, refers to the nucleic acid sequence or protein sequence of “class II major histocompatibility complex transactivator;” the human gene has accession number NC_000016.10 (range 10866208 . . . 10941562), reference GRCh38.p13. The CIITA protein in the nucleus acts as a positive regulator of MHC class II gene transcription and is required for MHC class II protein expression.

As used herein, “MHC” or “MHC molecule(s)” or “MHC protein” or “MHC complex(es),” refers to a major histocompatibility complex molecule (or plural), and includes, e.g., MHC class I and MHC class II molecules. In humans, MHC molecules are referred to as “human leukocyte antigen” complexes or “HLA molecules” or “HLA protein.” The use of terms “MHC” and “HLA” are not meant to be limiting; as used herein, the term “MHC” may be used to refer to human MHC molecules, i.e., HLA molecules. Therefore, the terms “MHC” and “HLA” are used interchangeably herein.

The term “HLA-A,” as used herein in the context of HLA-A protein, refers to the MHC class I protein molecule, which is a heterodimer consisting of a heavy chain (encoded by the HLA-A gene) and a light chain (i.e., beta-2 microglobulin). The term “HLA-A” or “HLA-A gene,” as used herein in the context of nucleic acids refers to the gene encoding the heavy chain of the HLA-A protein molecule. The HLA-A gene is also referred to as “HLA class I histocompatibility, A alpha chain;” the human gene has accession number NC_000006.12 (29942532 . . . 29945870). The HLA-A gene is known to have thousands of different versions (also referred to as “alleles”) across the population (and an individual may receive two different alleles of the HLA-A gene). A public database for HLA-A alleles, including sequence information, may be accessed at IPD-IMGT/HLA: https://www.ebi.ac.uk/ipd/imgt/hla/. All alleles of HLA-A are encompassed by the terms “HLA-A” and “HLA-A gene.”

“HLA-B” as used herein in the context of nucleic acids refers to the gene encoding the heavy chain of the HLA-B protein molecule. The HLA-B is also referred to as “HLA class I histocompatibility, B alpha chain;” the human gene has accession number NC_000006.12 (31353875 . . . 31357179).

“HLA-C” as used herein in the context of nucleic acids refers to the gene encoding the heavy chain of the HLA-C protein molecule. The HLA-C is also referred to as “HLA class I histocompatibility, C alpha chain;” the human gene has accession number NC_000006.12 (31268749 . . . 31272092).

“TRBC1” and “TRBC2” as used herein in the context of nucleic acids refer to two homologous genes encoding the T-cell receptor β-chain. “TRBC” or “TRBC1/2” is used herein to refer to TRBC1 and TRBC2. The human wild-type TRBC1 sequence is available at NCBI Gene ID: 28639; Ensembl: ENSG00000211751. T-cell receptor Beta Constant, V_segment Translation Product, BV05S1J2.2, TCRBC1, and TCRB are gene synonyms for TRBC1. The human wild-type TRBC2 sequence is available at NCBI Gene ID: 28638; Ensembl: ENSG00000211772. T-cell receptor Beta Constant, V_segment Translation Product, and TCRBC2 are gene synonyms for TRBC2.

“TRAC” is used to refer to the nucleic acid sequence or amino acid sequence of the “T cell receptor α chain”. A human wild-type TRAC sequence is available at NCBI Gene ID: 28755; Ensembl: ENSG00000277734. T-cell receptor Alpha Constant, TCRA, IMD7, TRCA and TRA are gene synonyms for TRAC.

“TRBC” is used to refer to the nucleic acid sequence or amino acid sequence of the “T-cell receptor β-chain”, e.g., TRBC1 and TRBC2. “TRBC1” and “TRBC2” refer to two homologous genes encoding the T-cell receptor β-chain, which are the gene products of the TRBC1 or TRBC2 genes.

A human wild-type TRBC1 sequence is available at NCBI Gene ID: 28639; Ensembl: ENSG00000211751. T-cell receptor Beta Constant, V_segment Translation Product, BV05S1J2.2, TCRBC1, and TCRB are gene synonyms for TRBC1.

A human wild-type TRBC2 sequence is available at NCBI Gene ID: 28638; Ensembl: ENSG00000211772. T-cell receptor Beta Constant, V_segment Translation Product, and TCRBC2 are gene synonyms for TRBC2.

As used herein, the term “homozygous” refers to having two identical alleles of a particular gene.

As used herein, “treatment” refers to any administration or application of a therapeutic for disease or disorder in a subject, and includes inhibiting the disease, arresting its development, relieving one or more symptoms of the disease, curing the disease, or preventing one or more symptoms of the disease, including reoccurrence of the symptom.

As used herein, “delivering” and “administering” are used interchangeably, and include ex vivo and in vivo applications.

Co-administration, as used herein, means that a plurality of substances are administered sufficiently close together in time so that the agents act together. Co-administration encompasses administering substances together in a single formulation and administering substances in separate formulations close enough in time so that the agents act together.

As used herein, the phrase “pharmaceutically acceptable” means that which is useful in preparing a pharmaceutical composition that is generally non-toxic and is not biologically undesirable and that are not otherwise unacceptable for pharmaceutical use. Pharmaceutically acceptable generally refers to substances that are non-pyrogenic. Pharmaceutically acceptable can refer to substances that are sterile, especially for pharmaceutical substances that are for injection or infusion.

As used herein, a “subject” refers to any member of the animal kingdom. In some embodiments, “subject” refers to humans. In some embodiments, “subject” refers to non-human animals. In some embodiments, “subject” refers to primates. In some embodiments, subjects include, but are not limited to, mammals, birds, reptiles, amphibians, fish, insects, or worms. In certain embodiments, the non-human subject is a mammal (e.g., a rodent, a mouse, a rat, a rabbit, a monkey, a dog, a cat, a sheep, cattle, a primate, or a pig). In some embodiments, a subject may be a transgenic animal, genetically-engineered animal, or a clone. In certain embodiments of the present invention the subject is an adult, an adolescent, or an infant. In some embodiments, terms “individual” or “patient” are used and are intended to be interchangeable with “subject”.

As used herein, “reduced or eliminated” expression of a protein on a cell refers to a partial or complete loss of expression of the protein relative to an unmodified cell. In some embodiments, the surface expression of a protein on a cell is measured by flow cytometry and has “reduced or eliminated” surface expression relative to an unmodified cell as evidenced by a reduction in fluorescence signal upon staining with the same antibody against the protein. A cell that has “reduced or eliminated” surface expression of a protein by flow cytometry relative to an unmodified cell may be referred to as “negative” for expression of that protein as evidenced by a fluorescence signal similar to a cell stained with an isotype control antibody. The “reduction or elimination” of protein expression can be measured by other known techniques in the field with appropriate controls known to those skilled in the art. As used herein, “eliminated” expression is understood as a reduction of expression to below the level of detection of the protein by the method used.

The term “about” or “approximately” means an acceptable error for a particular value as determined by one of ordinary skill in the art, which depends in part on how the value is measured or determined, or a degree of variation that does not substantially affect the properties of the described subject matter, or within the tolerances accepted in the art, e.g., within 10%, 5%, 2%, or 1% or within two standard deviations of a set of values. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

Reference will now be made in detail to certain embodiments of the invention, examples of which are illustrated in the accompanying drawings. While the invention is described in conjunction with the illustrated embodiments, it will be understood that they are not intended to limit the invention to those embodiments. On the contrary, the invention is intended to cover all alternatives, modifications, and equivalents, which may be included within the invention as defined by the appended claims and included embodiments.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The term “or” is used in an inclusive sense, i.e., equivalent to “and/or,” unless the context clearly indicates otherwise.

The section headings used herein are for organizational purposes only and are not to be construed as limiting the desired subject matter in any way. In the event that any material incorporated by reference contradicts any term defined in this specification or any other express content of this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### II. FIRST GENOME EDITING TOOL

In some embodiments, the first genome editing tool comprises a first genomic editor and at least one guide RNA (gRNA) that targets at least one genomic locus and that is cognate to the first genomic editor. In some embodiments, the first genome editing tool comprises a first genomic editor comprising a base editor, and at least one guide RNA (gRNA) that targets at least one genomic locus and that is cognate to the base editor.

In some embodiments, the first genomic editor is delivered to the cell as at least one polypeptide or at least one mRNA. In some embodiments, the first genomic editor comprises at least one polypeptide or at least one mRNA. In some embodiments, the first genomic editor comprises a cleavase, a nickase, a catalytically inactive nuclease, a base editor, optionally a C to T base editor or an A to G base editor, or a fusion protein comprising a DNA polymerase and a nickase.

In some embodiments, the first genomic editor comprises a Cas nuclease. In some embodiments, the Cas nuclease is a Cas9. In some embodiments, the Cas9 is Streptococcus pyogenes Cas9 (SpyCas9), S. aureus Cas9 (SauCas9), C. diphtheriae Cas9 (CdiCas9), Streptococcus thermophilus Cas9 (St1Cas9), A. cellulolyticus Cas9 (AceCas9), C. jejuni Cas9 (CjeCas9). R. palustris Cas9 (RpaCas9), R. rubrum Cas9 (RruCas9), A. naeslundii Cas9 (AnaCas9), Francisella novicida Cas9 (FnoCas9), or N. meningitidis (NmeCas9). In some embodiments, the Cas9 is an Nme1Cas9, an Nme2Cas9, an Nme3Cas9, or SpyCas9. In some embodiments, the Cas nuclease is a Class 2 Cas nuclease. In some embodiments, the Cas nuclease is a Cas12. In some embodiments, the Cas12 is Lachnospiraceae bacterium Cas12a (LbCas12a) or the Cas12 is Acidaminococcus sp. Cas12a (AsCas12a). In some embodiments, the Cas nuclease is an Eubacterium siraeum Cas13d (EsCas13d).

In some embodiments, the first genomic editor or the base editor comprises a cytidine deaminase (e.g., A3A). In some embodiments, the first genomic editor or the base editor comprises a cytidine deaminase (including any one of the cytidine deaminases disclosed herein, e.g., A3A), and an RNA-guided nickase (including any one of the RNA-guided nickases disclosed herein). In some embodiments, the base editor is a C to T base editor, optionally comprising a cytidine deaminase, or an A to G base editor, optionally comprising an adenosine deaminase.

In some embodiments, the first genomic editing tool may be combined with any second genomic editing tool disclosed herein.

### A. UGI

In some embodiments, the first genome editing tool comprises a uracil glycosylase inhibitor (UGI), and the UGI and the base editor are comprised in a single polypeptide. In some embodiments, the first genome editing tool comprises a UGI, and the UGI and the base editor are comprised in different polypeptides. In some embodiments, the base editor comprises a cytidine deaminase and an RNA-guided nickase. In some embodiments, the cytidine deaminase, the RNA-guided nickase, and the UGI are comprised in a single polypeptide. In some embodiments, the cytidine deaminase, the RNA-guided nickase, and the UGI are comprised in different polypeptides. In some embodiments, the cytidine deaminase and the RNA-guided nickase are comprised in a single polypeptide, and wherein the UGI is comprised in a different polypeptide.

Without being bound by any theory, providing a UGI together with a polypeptide comprising a deaminase may be helpful in the methods described herein by inhibiting cellular DNA repair machinery (e.g., UDG and downstream repair effectors) that recognize a uracil in DNA as a form of DNA damage or otherwise would excise or modify the uracil and/or surrounding nucleotides. It should be understood that the use of a UGI may increase the editing efficiency of an enzyme that is capable of deaminating C residues.

Suitable UGI protein and nucleotide sequences are provided herein and additional suitable UGI sequences are known to those in the art, and include, for example, those published in Wang et al., Uracil-DNA glycosylase inhibitor gene of bacteriophage PBS2 encodes a binding protein specific for uracil-DNA glycosylase. J. Biol. Chem. 264: 1163-1171(1989); Lundquist et al., Site-directed mutagenesis and characterization of uracil-DNA glycosylase inhibitor protein. Role of specific carboxylic amino acids in complex formation with Escherichia coli uracil-DNA glycosylase. J. Biol. Chem. 272:21408-21419(1997); Ravishankar et al., X-ray analysis of a complex of Escherichia coli uracil DNA glycosylase (EcUDG) with a proteinaceous inhibitor. The structure elucidation of a prokaryotic UDG. Nucleic Acids Res. 26:4880-4887(1998); and Putnam et al., Protein mimicry of DNA from crystal structures of the uracil-DNA glycosylase inhibitor protein and its complex with Escherichia coli uracil-DNA glycosylase. J. Mol. Biol. 287:331-346(1999), the entire contents of each are incorporated herein by reference. It should be appreciated that any proteins that are capable of inhibiting a uracil-DNA glycosylase base-excision repair enzyme are within the scope of the present disclosure. Additionally, any proteins that block or inhibit base-excision repair are also within the scope of this disclosure. In some embodiments, a uracil glycosylase inhibitor is a protein that binds uracil. In some embodiments, a uracil glycosylase inhibitor is a protein that binds uracil in DNA. In some embodiments, a uracil glycosylase inhibitor is a single-stranded binding protein. In some embodiments, a uracil glycosylase inhibitor is a catalytically inactive uracil DNA-glycosylase protein. In some embodiments, a uracil glycosylase inhibitor is a catalytically inactive uracil DNA-glycosylase protein that does not excise uracil from the DNA. In some embodiments, a uracil glycosylase inhibitor is a catalytically inactive UDG.

In some embodiments, a uracil glycosylase inhibitor (UGI) disclosed herein comprises an amino acid sequence with at least 80% to SEQ ID NO: 15 or 24. In some embodiments, any of the foregoing levels of identity is at least 90%, at least 95%, at least 98%, at least 99%, or 100%. In some embodiments, the UGI comprises an amino acid sequence with at least 90% identity to SEQ ID NO: 15 or 24. In some embodiments, the UGI comprises an amino acid sequence with at least 95% identity to SEQ ID NO: 15 or 24. In some embodiments, the UGI comprises an amino acid sequence with at least 98% identity to SEQ ID NO: 15 or 24. In some embodiments, the UGI comprises an amino acid sequence with at least 99% identity to SEQ ID NO: 15 or 24. In some embodiments, the UGI comprises the amino acid sequence of SEQ ID NO: 15 or 24.

### B. Cytidine Deaminase

Cytidine deaminases encompass enzymes in the cytidine deaminase superfamily, and in particular, enzymes of the APOBEC family (APOBEC1, APOBEC2, APOBEC4, and APOBEC3 subgroups of enzymes), activation-induced cytidine deaminase (AID or AICDA) and CMP deaminases (see, e.g., Conticello et al., Mol. Biol. Evol. 22:367-77, 2005; Conticello, Genome Biol. 9:229, 2008; Muramatsu et al., J. Biol. Chem. 274: 18470-6, 1999); and Carrington et al., Cells 9:1690 (2020)).

In some embodiments, the cytidine deaminase disclosed herein is an enzyme of APOBEC family. In some embodiments, the cytidine deaminase disclosed herein is an enzyme of APOBEC1, APOBEC2, APOBEC4, and APOBEC3 subgroups. In some embodiments, the cytidine deaminase disclosed herein is an enzyme of APOBEC3 subgroup. In some embodiments, the cytidine deaminase disclosed herein is an APOBEC3A deaminase (A3A).

In some embodiments, the cytidine deaminase is a cytidine deaminase comprising an amino acid sequence having at least 80%, 85% 87%, 90%, 95%, 98%, 99%, or 100% identity to SEQ ID NO: 22.

### 1. APOBEC3A Deaminase

In some embodiments, an APOBEC3A deaminase (A3A) disclosed herein is a human A3A. In some embodiments, the A3A is a wild-type A3A.

In some embodiment, the A3A is an A3A variant. A3A variants share homology to wild-type A3A, or a fragment thereof. In some embodiments, a A3A variant has at least about 80% identity, at least about 85% identity, at least about 90% identity, at least about 95% identity, at least about 96% identity, at least about 97% identity, at least about 98% identity, at least about 99% identity, at least about 99.5% identity, or at least about 99.9% identity to a wild type A3A. In some embodiments, the A3A variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a wild type A3A. In some embodiments, the A3A variant comprises a fragment of an A3A, such that the fragment has at least about 80% identity, at least about 90% identity, at least about 95% identity, at least about 96% identity, at least about 97% identity, at least about 98% identity, at least about 99% identity, at least about 99.5% identity, or at least about 99.9% identity to the corresponding fragment of a wild-type A3A.

In some embodiments, an A3A variant is a protein having a sequence that differs from a wild-type A3A protein by one or several mutations, such as substitutions, deletions, insertions, one or several single point substitutions. In some embodiments, a shortened A3A sequence could be used, e.g., by deleting N-terminal, C-terminal, or internal amino acids. In some embodiments, a shortened A3A sequence is used where one to four amino acids at the C-terminus of the sequence is deleted. In some embodiments, an APOBEC3A (such as a human APOBEC3A) has a wild-type amino acid position 57 (as numbered in the wild-type sequence). In some embodiments, an APOBEC3A (such as a human APOBEC3A) has an asparagine at amino acid position 57 (as numbered in the wild-type sequence).

In some embodiments, the wild-type A3A is a human A3A (UniPROT accession ID: p319411, SEQ ID NO: 22).

In some embodiments, the A3A disclosed herein comprises an amino acid sequence having at least 80% identity to SEQ ID NO: 22. In some embodiments, the level of identity is at least 85%, at least 87%, at least 90%, at least 95%, at least 98%, at least 99%, or 100%. In some embodiments, the A3A comprises an amino acid sequence having at least 87% identity to SEQ ID NO: 22. In some embodiments, the A3A comprises an amino acid sequence with at least 90% identity to SEQ ID NO: 22. In some embodiments, the A3A comprises an amino acid sequence with at least 95% identity to SEQ ID NO: 22. In some embodiments, the A3A comprises an amino acid sequence with at least 98% identity to SEQ ID NO: 22. In some embodiments, the A3A comprises an amino acid sequence with at least 99% identity to A3A SEQ ID NO: 22. In some embodiments, the A3A comprises the amino acid sequence of SEQ ID NO: 22.

### C. Linkers

In some embodiments, the first genomic editor or the base editor described herein further comprises a linker that connects the deaminase and the RNA-guided nickase. In some embodiments, the linker is an organic molecule, polymer, or chemical moiety. In some embodiments, the linker is a peptide linker. In some embodiments, the nucleic acid encoding the polypeptide comprising the deaminase and the RNA-guided nickase further comprises a sequence encoding the peptide linker. mRNAs encoding the deaminase-linker-RNA-guided nickase fusion protein are provided.

In some embodiments, the peptide linker is any stretch of amino acids having at least 1, at least 2, at least 3, at least 4, at least 5, at least 6, at least 7, at least 8, at least 9, at least 10, at least 15, at least 20, at least 25, at least 30, at least 40, at least 50, or more amino acids.

In some embodiments, the peptide linker is the 16 residue “XTEN” linker, or a variant thereof (See, e.g., Schellenberger et al. A recombinant polypeptide extends the in vivo half-life of peptides and proteins in a tunable manner. Nat. Biotechnol. 27, 1186-1190 (2009)). In some embodiments, the XTEN linker comprises a sequence that is any one of SGSETPGTSESATPES (SEQ ID NO: 25), SGSETPGTSESA (SEQ ID NO: 26), or SGSETPGTSESATPEGGSGGS (SEQ ID NO: 27). In some embodiments, the XTEN linker consists of the sequence SGSETPGTSESATPES (SEQ ID NO: 25), SGSETPGTSESA (SEQ ID NO: 26), or SGSETPGTSESATPEGGSGGS (SEQ ID NO: 27).

In some embodiments, the peptide linker comprises a (GGGGS)ₙ (e.g., SEQ ID NOs: 73, 77, 82, 101), a (G)ₙ, an (EAAAK)ₙ (e.g., SEQ ID NOs: 74, 80, 128), a (GGS)ₙ, an SGSETPGTSESATPES (SEQ ID NO: 25) motif (see, e.g., Guilinger J P, Thompson D B, Liu D R. Fusion of catalytically inactive Cas9 to FokI nuclease improves the specificity of genome modification. Nat. Biotechnol. 2014; 32(6): 577-82; the entire contents are incorporated herein by reference), or an (XP)ₙ motif (SEQ ID NO: 407), or a combination of any of these, wherein n is independently an integer between 1 and 30. See, WO2015089406, e.g., paragraph [0012], the entire content of which is incorporated herein by reference.

In some embodiments, the peptide linker comprises one or more sequences selected from SEQ ID NOs: 25-39 and 72-133. In some embodiments, the peptide linker comprises one or more sequences selected from SEQ ID NO: 25, SEQ ID NO: 26, SEQ ID NO: 27, SEQ ID NO: 129, SEQ ID NO: 130, SEQ ID NO: 131. SEQ ID NO: 132 and SEQ ID NO: 133. In some embodiments, the peptide linker comprises a sequence of SEQ ID NO: 129.

### D. RNA-Guided Nickase

In some embodiments, an RNA-guided nickase disclosed herein is a Cas nickase. In some embodiments, an RNA-guided nickase is from a specific Cas nuclease with its catalytic domain(s) being inactivated. In some embodiments, the RNA-guided nickase is a Class 2 Cas nickase, such as a Cas9 nickase or a Cpf1 nickase. In some embodiments, the RNA-guided nickase is an S. pyogenes Cas9 nickase. In some embodiments, the RNA-guided nickase is Neisseria meningitidis Cas9 nickase.

In some embodiments, the RNA-guided nickase is a modified Class 2 Cas protein or derived from a Class 2 Cas protein. In some embodiments, the RNA-guided nickase is modified or derived from a Cas protein, such as a Class 2 Cas nuclease (which may be, e.g., a Cas nuclease of Type II, V, or VI). Class 2 Cas nuclease include, for example, Cas9, Cpf1 (Cas12a), C2c1, C2c2, and C2c3 proteins and modifications thereof. Examples of Cas9 nucleases include those of the type II CRISPR systems of S. pyogenes, S. aureus, and other prokaryotes (see, e.g., the list in the next paragraph), and modified (e.g., engineered or mutant) versions thereof. See, e.g., US2016/0312198 A1; US 2016/0312199 A1, which is incorporated by reference in its entirety. Other examples of Cas nucleases include a Csm or Cmr complex of a type III CRISPR system or the Cas10, Csm1, or Cmr2 subunit thereof, and a Cascade complex of a type I CRISPR system, or the Cas3 subunit thereof. In some embodiments, the Cas nuclease may be from a Type-IIA, Type-IIB, or Type-IIC system. For discussion of various CRISPR systems and Cas nucleases, see, e.g., Makarova et al., NAT. REV. MICROBIOL. 9:467-477 (2011); Makarova et al., NAT. REV. MICROBIOL, 13: 722-36 (2015); Shmakov et al., MOLECULAR CELL, 60:385-397 (2015).

A Cas nickase described herein may be a nickase form of a Cas nuclease from the species including, but not limited to, Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gammaproteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, Acidaminococcus sp., Lachnospiraceae bacterium ND2006, or Acaryochloris marina.

In some embodiments, the Cas nickase is a nickase form of the Cas9 nuclease from Streptococcus pyogenes. In some embodiments, the Cas nickase is a nickase form of the Cas9 nuclease from Streptococcus thermophilus. In some embodiments, the Cas nickase is a nickase form of the Cas9 nuclease from Neisseria meningitidis. See e.g., WO/2020081568, describing an Nme2Cas9 D16A nickase. In some embodiments, the Cas nickase is a nickase form of the Cas9 nuclease from Staphylococcus aureus. In some embodiments, the Cas nickase is a nickase form of the Cpf1 nuclease from Francisella novicida. In some embodiments, the Cas nickase is a nickase form of the Cpf1 nuclease from Acidaminococcus sp. In some embodiments, the Cas nickase is a nickase form of the Cpf1 nuclease from Lachnospiraceae bacterium ND2006. In further embodiments, the Cas nickase is a nickase form of the Cpf1 nuclease from Francisella tularensis, Lachnospiraceae bacterium, Butyrivibrio proteoclasticus, Peregrinibacteria bacterium, Parcubacteria bacterium, Smithella, Acidaminococcus, Candidatus Methanoplasma termitum, Eubacterium eligens, Moraxella bovoculi, Leptospira inadai, Porphyromonas crevioricanis, Prevotella disiens, or Porphyromonas macacae. In certain embodiments, the Cas nickase is a nickase form of a Cpf1 nuclease from an Acidaminococcus or Lachnospiraceae. As discussed elsewhere, a nickase may be derived from (i.e., related to) a specific Cas nuclease in that the nickase is a form of the nuclease in which one of its two catalytic domains is inactivated, e.g., by mutating an active site residue essential for nucleolysis, such as D10, H840, or N863 in Spy Cas9. One skilled in the art will be familiar with techniques for easily identifying corresponding residues in other Cas proteins, such as sequence alignment and structural alignment, which is discussed in detail below.

In other embodiments, the Cas nickase may relate to a Type-I CRISPR/Cas system. In some embodiments, the Cas nickase may be a component of the Cascade complex of a Type-I CRISPR/Cas system. In some embodiments, the Cas nickase may be a Cas3 protein. In some embodiments, the Cas nickase may be from a Type-III CRISPR/Cas system.

In some embodiments, a Cas nickase is a nickase form of a Cas nuclease or a modified Cas nuclease in which an endonucleolytic active site is inactivated, e.g., by one or more alterations (e.g., point mutations) in a catalytic domain. See, e.g., U.S. Pat. No. 8,889,356 for discussion of Cas nickases and exemplary catalytic domain alterations.

Wild type S. pyogenes Cas9 has two catalytic domains: RuvC and HNH. The RuvC domain cleaves the non-target DNA strand, and the HNH domain cleaves the target strand of DNA. In some embodiments, a Cas nuclease may comprise an amino acid substitution in the RuvC or RuvC-like nuclease domain. Exemplary amino acid substitutions in the RuvC or RuvC-like nuclease domain include D10A (based on the S. pyogenes Cas9 protein). See, e.g., Zetsche et al. (2015) Cell Oct. 22:163(3): 759-771. In some embodiments, the Cas nuclease may comprise an amino acid substitution in the HNH or HNH-like nuclease domain. Exemplary amino acid substitutions in the HNH or HNH-like nuclease domain include E762A, H840A, N863A, H983A, and D986A (based on the S. pyogenes Cas9 protein). See, e.g., Zetsche et al. (2015). Exemplary amino acid substitutions in the HNH or HNH-like nuclease domain or RuvC or RuvC-like domains for N. meningitidis include Nme2Cas9D16A (HNH nickase) and Nme2Cas9H588A (RuvC nickase). Further exemplary amino acid substitutions include D917A, E1006A, and D1255A (based on the Francisella novicida U112 Cpf1 (FnCpf1) sequence (UniProtKB-AOQ7Q2 (CPF1_FRATN)).

In some embodiments, a Cas nickase such as a Cas9 nickase has an inactivated RuvC or HNH domain. In some embodiments, a nickase is used having a RuvC domain with reduced activity. In some embodiments, a nickase is used having an inactive RuvC domain.

In some embodiments, a nickase is used having an HNH domain with reduced activity. In some embodiments, a nickase is used having an inactive HNH domain.

In some embodiments, a Cas9 nickase has an active HNH nuclease domain and is able to cleave the non-targeted strand of DNA, i.e., the strand bound by the gRNA and has an inactive RuvC nuclease domain and is not able to cleave the targeted strand of the DNA, i.e., the strand where base editing by deaminase is desired.

An exemplary Cas9 nickase amino acid sequence is provided as SEQ ID NO: 41. An exemplary Cas9 nickase mRNA coding sequence, suitable for inclusion in a fusion protein, is provided as SEQ ID NO: 42.

In some embodiments, the RNA-guided nickase is a Class 2 Cas nickase described herein. In some embodiments, the RNA-guided nickase is a Cas9 nickase described herein.

In some embodiments, the RNA-guided nickase is an S. pyogenes Cas9 nickase described herein.

In some embodiments, the RNA-guided nickase is a D10A SpyCas9 nickase described herein. In some embodiments, the RNA-guided nickase comprises an amino acid sequence having at least 80%, 90%, 95%, 98%, or 99% identity to any one of SEQ ID NO: 41, 43, or 45. In some embodiments, the RNA-guided nickase comprises the amino acid sequence of SEQ ID NO: 41.

In some embodiments, the nucleic acid or the first ORF encoding the polypeptide comprises a nucleotide sequence having at least 80%, 90%, 95%, 98%, 99% or 100% identity to the nucleotide sequence of any one of SEQ ID NOs: 42, 44, or 46. In some embodiments, the nucleic acid or the first ORF encoding the polypeptide comprises a nucleotide sequence having at least 80%, 90%, 95%, 98%, 99% or 100% identity to the nucleotide sequence of any one of SEQ ID NOs: 42, 44, and 46-58. In some embodiments, the level of identity is at least 90%. In some embodiments, the level of identity is at least 95%. In some embodiments, the level of identity is at least 98%. In some embodiments, the level of identity is at least 99%. In some embodiments, the level of identity is at least 100%. In some embodiments, the sequence encoding the RNA-guided nickase comprises the nucleotide sequence of any one of SEQ ID NOs: 42, 44, and 46.

In some embodiments, the RNA-guided nickase is Neisseria meningitidis (Nine) Cas9 nickase described herein.

In some embodiments, the RNA-guided nickase is a D16A NmeCas9 nickase described herein. In some embodiments, the D16A NmeCas9 nickase is a D16A Nme2Cas9 nickase. In some embodiments, the D16A Nme2Cas9 nickase comprises an amino acid sequence at least 80%, 90%, 95%, 98%, 99% or 100% identical to SEQ ID NO: 149. In some embodiments, the sequence encoding the D16A Nme2Cas9 comprises a nucleotide sequence at least 80%, 90%, 95%, 98%, 99% or 100% identical to any one of SEQ ID NOs: 150-155.

### E. Compositions Comprising a Cytidine Deaminase and an RNA-Guided Nickase

In some embodiments, the first genome editing tool comprises a first genomic editor and at least one guide RNA (gRNA) that targets at least one genomic locus and that is cognate to the first genomic editor. In some embodiments, the first genome editing tool comprises a first genomic editor comprising a base editor, and at least one guide RNA (gRNA) that targets at least one genomic locus and that is cognate to the base editor.

In some embodiments, the first genome editing tool comprises a uracil glycosylase inhibitor (UGI), and the UGI and the base editor are comprised in a single polypeptide. In some embodiments, the first genome editing tool comprises a UGI, and the UGI and the base editor are comprised in different polypeptides. In some embodiments, the base editor comprises a cytidine deaminase and an RNA-guided nickase. In some embodiments, the cytidine deaminase, the RNA-guided nickase, and the UGI are comprised in a single polypeptide. In some embodiments, the cytidine deaminase, the RNA-guided nickase, and the UGI are comprised in different polypeptides. In some embodiments, the cytidine deaminase and the RNA-guided nickase are comprised in a single polypeptide, and wherein the UGI is comprised in a different polypeptide.

### 1. Exemplary Compositions

In some embodiments, a first genomic editor (e.g., base editor) comprising a deaminase (e.g., a cytidine deaminase) and an RNA-guided nickase is provided. In some embodiments, an enzyme of APOBEC family and an RNA-guided nickase is provided. In some embodiments, the first genomic editor comprises an enzyme of APOBEC1 subgroup and an RNA-guided nickase. In some embodiments, the first genomic editor comprises an enzyme of APOBEC2 subgroup and an RNA-guided nickase. In some embodiments, the first genomic editor comprises an enzyme of APOBEC4 subgroup and an RNA-guided nickase. In some embodiments, the first genomic editor comprises an enzyme of APOBEC3 subgroup and an RNA-guided nickase.

In some embodiments, a first genomic editor or a base editor comprising a deaminase (e.g., a cytidine deaminase) and an RNA-guided nickase is provided. In some embodiments, an enzyme of APOBEC family and a D10A SpyCas9 nickase is provided. In some embodiments, the first genomic editor comprises an enzyme of APOBEC1 subgroup and a D10A SpyCas9 nickase. In some embodiments, the first genomic editor comprises an enzyme of APOBEC2 subgroup and a D10A SpyCas9 nickase. In some embodiments, the first genomic editor comprises an enzyme of APOBEC4 subgroup and a D10A SpyCas9 nickase. In some embodiments, the first genomic editor comprises an enzyme of APOBEC3 subgroup and a D10A SpyCas9 nickase.

In some embodiments, a first genomic editor or a base editor comprising a deaminase (e.g., a cytidine deaminase) and an RNA-guided nickase is provided. In some embodiments, an enzyme of APOBEC family and a D16A NmeCas9 nickase is provided. In some embodiments, an enzyme of APOBEC family and a D16A Nme2Cas9 nickase is provided. In some embodiments, the first genomic editor comprises an enzyme of APOBEC1 subgroup and a D16A Nme2Cas9 nickase. In some embodiments, the first genomic editor comprises an enzyme of APOBEC2 subgroup and a D16A Nme2Cas9 nickase. In some embodiments, the first genomic editor comprises an enzyme of APOBEC4 subgroup and a D16A Nme2Cas9 nickase. In some embodiments, the first genomic editor comprises an enzyme of APOBEC3 subgroup and a D16A Nme2Cas9 nickase.

In some embodiments, the first genomic editor lacks a UGI. In some embodiments, the first genomic editor contains one or more UGIs.

In some embodiments, the cytidine deaminase and the RNA-guided nickase are linked via a linker. In some embodiments, the cytidine deaminase and the RNA-guided nickase are linked via a peptide linker. In some embodiments, the peptide linker comprises one or more sequences selected from SEQ ID NOs: 25-39 and 72-133.

In some embodiments, the first genomic editor further comprises one or more additional heterologous functional domains. In some embodiments, the first genomic editor further comprises one or more nuclear localization sequences (NLSs) (described herein) at the C-terminal of the polypeptide or the N-terminal of the polypeptide.

In some embodiments, a first genomic editor or a base editor comprising a deaminase (e.g., a cytidine deaminase) and an RNA-guided nickase is provided. In some embodiments, an enzyme of APOBEC family and an RNA-guided nickase is provided. In some embodiments, the first genomic editor comprises an enzyme of APOBEC1 subgroup and an RNA-guided nickase. In some embodiments, the first genomic editor comprises an enzyme of APOBEC2 subgroup and an RNA-guided nickase. In some embodiments, the first genomic editor comprises an enzyme of APOBEC4 subgroup and an RNA-guided nickase. In some embodiments, the first genomic editor comprises an enzyme of APOBEC3 subgroup and an RNA-guided nickase.

In some embodiments, a first genomic editor or a base editor comprising a deaminase (e.g., a cytidine deaminase) and an RNA-guided nickase is provided. In some embodiments, an enzyme of APOBEC family and a D10A SpyCas9 nickase, wherein the enzyme of APOBEC family and the D10A SpyCas9 nickase are fused via a linker. In some embodiments, the first genomic editor comprises an enzyme of APOBEC family and a D10A SpyCas9 nickase, and a nuclear localization sequence (NLS) at the C-terminus of the fused polypeptide. In some embodiments, the first genomic editor comprises an enzyme of APOBEC family and a D10A SpyCas9 nickase, and a NLS at the N-terminus of the fused polypeptide. In some embodiments, the first genomic editor comprises an enzyme of APOBEC family and a D10A SpyCas9 nickase, wherein the enzyme of APOBEC family and the D10A SpyCas9 nickase are fused via a linker, and a NLS fused to the C-terminus of the D10A SpyCas9 nickase, optionally via a linker. In some embodiments, the first genomic editor comprises an enzyme of APOBEC family and a D10A SpyCas9 nickase, wherein the enzyme of APOBEC family and the D10A SpyCas9 nickase are fused via a linker, and a NLS fused to the C-terminus of the D10A SpyCas9 nickase, optionally via a linker.

In some embodiments, the first genomic editor comprises an enzyme of APOBEC family and a D16A NmeCas9 nickase, wherein the enzyme of APOBEC family and the D16A NmeCas9 nickase are fused via a linker. In some embodiments, the first genomic editor comprises an enzyme of APOBEC family and a D16A Nme2Cas9 nickase, wherein the enzyme of APOBEC family and the D16A Nme2Cas9 nickase are fused via a linker. In some embodiments, the first genomic editor comprises an enzyme of APOBEC family and a D16A Nme2Cas9 nickase, and a nuclear localization sequence (NLS) at the C-terminus of the fused polypeptide. In some embodiments, the first genomic editor comprises an enzyme of APOBEC family and a D16A Nme2Cas9 nickase, and a NLS at the N-terminus of the fused polypeptide. In some embodiments, the first genomic editor comprises an enzyme of APOBEC family and a D16A Nme2Cas9 nickase, wherein the enzyme of APOBEC family and the D16A Nme2Cas9 nickase are fused via a linker, and a NLS fused to the C-terminus of the D16A Nme2Cas9 nickase, optionally via a linker. In some embodiments, the first genomic editor comprises an enzyme of APOBEC family and a D16A Nme2Cas9 nickase, wherein the enzyme of APOBEC family and the D16A Nme2Cas9 nickase are fused via a linker, and a NLS fused to the C-terminus of the D16A Nme2Cas9 nickase, optionally via a linker.

In some embodiments, the first genomic editor comprises an enzyme of APOBEC1 subgroup and a D10A SpyCas9 nickase, wherein the enzyme of APOBEC1 subgroup and the D10A SpyCas9 nickase are fused via a linker. In some embodiments, the first genomic editor comprises an enzyme of APOBEC1 subgroup and a D10A SpyCas9 nickase, and a nuclear localization sequence (NLS) at the C-terminus of the fused polypeptide. In some embodiments, the first genomic editor comprises an enzyme of APOBEC1 subgroup and a D10A SpyCas9 nickase, and a NLS at the N-terminus of the fused polypeptide. In some embodiments, the first genomic editor comprises an enzyme of APOBEC1 subgroup and a D10A SpyCas9 nickase, wherein the enzyme of APOBEC1 subgroup and the D10A SpyCas9 nickase are fused via a linker, and a NLS fused to the C-terminus of the D10A SpyCas9 nickase, optionally via a linker. In some embodiments, the first genomic editor comprises an enzyme of APOBEC1 subgroup and a D10A SpyCas9 nickase, wherein the enzyme of APOBEC1 subgroup and the D10A SpyCas9 nickase are fused via a linker, and a NLS fused to the C-terminus of the D10A SpyCas9 nickase, optionally via a linker.

In some embodiments, the first genomic editor comprises an enzyme of APOBEC1 subgroup and a D16A Nme2Cas9 nickase, wherein the enzyme of APOBEC1 subgroup and the D16A Nme2Cas9 nickase are fused via a linker. In some embodiments, the first genomic editor comprises an enzyme of APOBEC1 subgroup and a D16A Nme2Cas9 nickase, wherein the enzyme of APOBEC1 subgroup and the D16A Nme2Cas9 nickase are fused via a linker. In some embodiments, the first genomic editor comprises an enzyme of APOBEC1 subgroup and a D16A Nme2Cas9 nickase, and a nuclear localization sequence (NLS) at the C-terminus of the fused polypeptide. In some embodiments, the first genomic editor comprises an enzyme of APOBEC1 subgroup and a D16A Nme2Cas9 nickase, and a NLS at the N-terminus of the fused polypeptide. In some embodiments, the first genomic editor comprises an enzyme of APOBEC1 subgroup and a D16A Nme2Cas9 nickase, wherein the enzyme of APOBEC1 subgroup and the D16A Nme2Cas9 nickase are fused via a linker, and a NLS fused to the C-terminus of the D16A Nme2Cas9 nickase, optionally via a linker. In some embodiments, the first genomic editor comprises an enzyme of APOBEC1 subgroup and a D16A Nme2Cas9 nickase, wherein the enzyme of APOBEC1 subgroup and the D16A Nme2Cas9 nickase are fused via a linker, and a NLS fused to the C-terminus of the D16A Nme2Cas9 nickase, optionally via a linker.

In some embodiments, the first genomic editor comprises an enzyme of APOBEC3 subgroup and a D10A SpyCas9 nickase, wherein the enzyme of APOBEC3 subgroup and the D10A SpyCas9 nickase are fused via a linker. In some embodiments, the first genomic editor comprises an enzyme of APOBEC3 subgroup and a D10A SpyCas9 nickase, and a nuclear localization sequence (NLS) at the C-terminus of the fused polypeptide. In some embodiments, the first genomic editor comprises an enzyme of APOBEC3 subgroup and a D10A SpyCas9 nickase, and a NLS at the N-terminus of the fused polypeptide. In some embodiments, the first genomic editor comprises an enzyme of APOBEC3 subgroup and a D10A SpyCas9 nickase, wherein the enzyme of APOBEC3 subgroup and the D10A SpyCas9 nickase are fused via a linker, and a NLS fused to the C-terminus of the D10A SpyCas9 nickase, optionally via a linker. In some embodiments, the first genomic editor comprises an enzyme of APOBEC3 subgroup and a D10A SpyCas9 nickase, wherein the enzyme of APOBEC3 subgroup and the D10A SpyCas9 nickase are fused via a linker, and a NLS fused to the C-terminus of the D10A SpyCas9 nickase, optionally via a linker.

In some embodiments, the first genomic editor comprises an enzyme of APOBEC3 subgroup and a D16A Nme2Cas9 nickase, wherein the enzyme of APOBEC3 subgroup and the D16A Nme2Cas9 nickase are fused via a linker. In some embodiments, the first genomic editor comprises an enzyme of APOBEC3 subgroup and a D16A Nme2Cas9 nickase, wherein the enzyme of APOBEC3 subgroup and the D16A Nme2Cas9 nickase are fused via a linker. In some embodiments, the first genomic editor comprises an enzyme of APOBEC3 subgroup and a D16A Nme2Cas9 nickase, and a nuclear localization sequence (NLS) at the C-terminus of the fused polypeptide. In some embodiments, the first genomic editor comprises an enzyme of APOBEC3 subgroup and a D16A Nme2Cas9 nickase, and a NLS at the N-terminus of the fused polypeptide. In some embodiments, the first genomic editor comprises an enzyme of APOBEC3 subgroup and a D16A Nme2Cas9 nickase, wherein the enzyme of APOBEC3 subgroup and the D16A Nme2Cas9 nickase are fused via a linker, and a NLS fused to the C-terminus of the D16A Nme2Cas9 nickase, optionally via a linker. In some embodiments, the first genomic editor comprises an enzyme of APOBEC3 subgroup and a D16A Nme2Cas9 nickase, wherein the enzyme of APOBEC3 subgroup and the D16A Nme2Cas9 nickase are fused via a linker, and a NLS fused to the C-terminus of the D16A Nme2Cas9 nickase, optionally via a linker.

In some embodiments, the first genomic editor comprises a D10A SpyCas9 nickase, a linker comprising the amino acid sequence of SEQ ID NO: 129, and a cytidine deaminase comprising an amino acid sequence that is at least 85% identical to SEQ ID NO: 22. In some embodiments, the first genomic editor comprises a D10A SpyCas9 nickase, a linker comprising the amino acid sequence of SEQ ID NO: 130, and a cytidine deaminase comprising an amino acid sequence that is at least 85% identical to SEQ ID NO: 22. In some embodiments, the first genomic editor comprises a D10A SpyCas9 nickase, a linker comprising the amino acid sequence of SEQ ID NO: 131, and a cytidine deaminase comprising an amino acid sequence that is at least 85% identical to SEQ ID NO: 22. In some embodiments, the first genomic editor comprises a D10A SpyCas9 nickase, a linker comprising the amino acid sequence of SEQ ID NO: 132, and a cytidine deaminase comprising an amino acid sequence that is at least 85% identical to SEQ ID NO: 22. In some embodiments, the first genomic editor comprises a D10A SpyCas9 nickase, a linker comprising the amino acid sequence of SEQ ID NO: 133, and a cytidine deaminase comprising an amino acid sequence that is at least 85% identical to SEQ ID NO: 22. In any of the foregoing embodiments, the D10A SpyCas9 nickase may comprise an amino acid sequence that is at least 85%, at least 90%, at least 95%, at least 98%, at least 99%, or 100% identical to any one of SEQ ID NOs: 41, 43, and 45.

In some embodiments, the first genomic editor comprises a D16A Nme2Cas9 nickase, a linker comprising the amino acid sequence of SEQ ID NO: 129, and a cytidine deaminase comprising an amino acid sequence that is at least 85% identical to SEQ ID NO: 22. In some embodiments, the first genomic editor comprises a D16A Nme2Cas9 nickase, a linker comprising the amino acid sequence of SEQ ID NO: 130, and a cytidine deaminase comprising an amino acid sequence that is at least 85% identical to SEQ ID NO: 22. In some embodiments, the first genomic editor comprises a D16A Nme2Cas9 nickase, a linker comprising the amino acid sequence of SEQ ID NO: 131, and a cytidine deaminase comprising an amino acid sequence that is at least 85% identical to SEQ ID NO: 22. In some embodiments, the first genomic editor comprises a D16A Nme2Cas9 nickase, a linker comprising the amino acid sequence of SEQ ID NO: 132, and a cytidine deaminase comprising an amino acid sequence that is at least 85% identical SEQ ID NO: 22. In some embodiments, the first genomic editor comprises a D16A Nme2Cas9 nickase, a linker comprising the amino acid sequence of SEQ ID NO: 133, and a cytidine deaminase comprising an amino acid sequence that is at least 85% identical to SEQ ID NO: 22. In any of the foregoing embodiments, the D16A Nme2Cas9 nickase may comprise an amino acid sequence that is at least 85%, at least 90%, at least 95%, at least 98%, at least 99%, or 100% identical to SEQ ID NO: 149.

In some embodiments, the first genomic editor comprises a D10A SpyCas9 nickase, a linker comprising the amino acid sequence of SEQ ID NO: 129, and a cytidine deaminase comprising the amino acid sequence of SEQ ID NO: 22. In some embodiments, the first genomic editor comprises a D10A SpyCas9 nickase, a linker comprising the amino acid sequence of SEQ ID NO: 130, and a cytidine deaminase comprising the amino acid sequence of SEQ ID NO: 22. In some embodiments, the first genomic editor comprises a D10A SpyCas9 nickase, a linker comprising the amino acid sequence of SEQ ID NO: 131, and a cytidine deaminase comprising the amino acid sequence of SEQ ID NO: 22. In some embodiments, the first genomic editor comprises a D10A SpyCas9 nickase, a linker comprising the amino acid sequence of SEQ ID NO: 132, and a cytidine deaminase comprising the amino acid sequence of SEQ ID NO: 22. In some embodiments, the first genomic editor comprises a D10A SpyCas9 nickase, a linker comprising the amino acid sequence of SEQ ID NO: 133, and a cytidine deaminase comprising the amino acid sequence of SEQ ID NO: 22. In any of the foregoing embodiments, the D10A SpyCas9 comprises an amino acid sequence that is at least 85%, at least 90%, at least 95%, at least 98%, at least 99%, or 100% identical to any one of SEQ ID NOs: 41, 43, and 45.

In some embodiments, the first genomic editor comprises a D16A Nme2Cas9 nickase, a linker comprising the amino acid sequence of SEQ ID NO: 129, and a cytidine deaminase comprising the amino acid sequence of SEQ ID NO: 22. In some embodiments, the first genomic editor comprises a D16A Nme2Cas9 nickase, a linker comprising the amino acid sequence of SEQ ID NO: 130, and a cytidine deaminase comprising the amino acid sequence of SEQ ID NO: 22. In some embodiments, the first genomic editor comprises a D16A Nme2Cas9 nickase, a linker comprising the amino acid sequence of SEQ ID NO: 131, and a cytidine deaminase comprising the amino acid sequence of SEQ ID NO: 22. In some embodiments, the first genomic editor comprises a D16A Nme2Cas9 nickase, a linker comprising the amino acid sequence of SEQ ID NO: 132, and a cytidine deaminase comprising the amino acid sequence of SEQ ID NO: 22. In some embodiments, the first genomic editor comprises a D16A Nme2Cas9 nickase, a linker comprising the amino acid sequence of SEQ ID NO: 133, and a cytidine deaminase comprising the amino acid sequence of SEQ ID NO: 22. In any of the foregoing embodiments, the D16A Nme2Cas9 nickase comprises an amino acid sequence that is at least 85%, at least 90%, at least 95%, at least 98%, at least 99%, or 100% identical to SEQ ID NO: 149.

The first genomic editor may be organized in any number of ways to form a single chain. The NLS can be N- or C-terminal, or both N- and C-terminals, and the cytidine deaminase can be N- or C-terminal as compared the RNA-guided nickase. In some embodiments, the first genomic editor comprises, from N to C terminus, a cytidine deaminase, an optional linker, an RNA-guided nickase, and an optional NLS. In some embodiments, the first genomic editor comprises, from N to C terminus, an RNA-guided nickase, an optional linker, a cytidine deaminase, and an optional NLS. In some embodiments, the first genomic editor comprises, from N to C terminus, an optional NLS, an RNA-guided nickase, an optional linker, and a cytidine deaminase. In some embodiments, the first genomic editor comprises, from N to C terminus, an optional NLS, an RNA-guided nickase, an optional linker, and a cytidine deaminase, and an optional NLS.

In some embodiments, the first genomic editor comprises, from N to C terminus, an optional NLS, an enzyme of APOBEC family, an optional linker, an RNA-guided nickase, and an optional NLS. In some embodiments, the first genomic editor comprises, from N to C terminus, an optional NLS, an RNA-guided nickase, an optional linker, an enzyme of APOBEC family and an optional NLS. In some embodiments, the first genomic editor comprises, from N to C terminus, an optional NLS, an RNA-guided nickase, an optional linker, an enzyme of APOBEC family, and an optional NLS. In some embodiments, the first genomic editor comprises, from N to C terminus, an optional NLS, an RNA-guided nickase, an optional linker, an enzyme of APOBEC family, and an optional NLS.

In some embodiments, the first genomic editor comprises, from N to C terminus, an optional NLS, an enzyme of APOBEC3 subgroup, an optional linker, an RNA-guided nickase, and an optional NLS. In some embodiments, the first genomic editor comprises, from N to C terminus, an optional NLS, an RNA-guided nickase, an optional linker, an enzyme of APOBEC3 subgroup and an optional NLS. In some embodiments, the first genomic editor comprises, from N to C terminus, an optional NLS, an RNA-guided nickase, an optional linker, an enzyme of APOBEC3 subgroup, and an optional NLS. In some embodiments, the first genomic editor comprises, from N to C terminus, an optional NLS, an RNA-guided nickase, an optional linker, an enzyme of APOBEC3 subgroup, and an optional NLS.

In some embodiments, the first genomic editor comprises, from N to C terminus, an optional NLS, an enzyme of APOBEC family, an optional linker, a D10A SpyCas9 nickase or a D16A Nme2Cas9 nickase, and an optional NLS. In some embodiments, the first genomic editor comprises, from N to C terminus, an optional NLS, a DTOA SpyCas9 nickase or a D16A Nme2Cas9 nickase, an optional linker, an enzyme of APOBEC family and an optional NLS. In some embodiments, the first genomic editor comprises, from N to C terminus, an optional NLS, a DTOA SpyCas9 nickase or a D16A Nme2Cas9 nickase, an optional linker, an enzyme of APOBEC family, and an optional NLS. In some embodiments, the first genomic editor comprises, from N to C terminus, an optional NLS, a DTOA SpyCas9 nickase or a D16A Nme2Cas9 nickase, an optional linker, and an enzyme of APOBEC family, and an optional NLS.

In some embodiments, the first genomic editor comprises, from N to C terminus, an optional NLS, an enzyme of APOBEC3 subgroup, an optional linker, a D10A SpyCas9 nickase or a D16A Nme2Cas9 nickase, and an optional NLS. In some embodiments, the first genomic editor comprises, from N to C terminus, an optional NLS, a DTOA SpyCas9 nickase or a D16A Nme2Cas9 nickase, an optional linker, an enzyme of APOBEC3 subgroup and an optional NLS. In some embodiments, the first genomic editor comprises, from N to C terminus, an optional NLS, a DTOA SpyCas9 nickase or a D16A Nme2Cas9 nickase, an optional linker, an enzyme of APOBEC3 subgroup, and an optional NLS. In some embodiments, the first genomic editor comprises, from N to C terminus, an optional NLS, a D10A SpyCas9 nickase or a D16A Nme2Cas9 nickase, an optional linker, and an enzyme of APOBEC3 subgroup, and an optional NLS.

In some embodiments, the first genomic editor comprises, from N to C terminus, an optional NLS, an enzyme of APOBEC3 subgroup, an optional linker, a D16A Nme2Cas9 nickase.

In some embodiments, the first genomic editor comprises, from N to C terminus, (i) an optional NLS; (ii) a cytidine deaminase comprising an amino acid sequence that is at least 80% identical to SEQ ID NOs: 22; (iii) a linker comprising one or more sequences selected from SEQ ID NOs: 25-38, 39 and 72-133, (iv) a D10A SpyCas9 nickase or a D16A Nme2Cas9 nickase, and (v) an optional NLS.

In some embodiments, the first genomic editor comprises, from N to C terminus, (i) an optional NLS, (ii) a D10A SpyCas9 nickase or a D16A Nme2Cas9 nickase, (iii) a linker comprising one or more sequences selected from SEQ ID NOs: 25-38, 39 and 72-133, (iv) a cytidine deaminase comprising an amino acid sequence that is at least 80% identical to SEQ ID NOs: 22, and (v) an optional NLS.

In some embodiments, the first genomic editor comprises, from N to C terminus, (i) an optional NLS, (ii) a D10A SpyCas9 nickase or a D16A Nme2Cas9 nickase, (iii) a linker comprising one or more sequences selected from SEQ ID NOs: 25-38, 39 and 72-133, (iv) a cytidine deaminase comprising an amino acid sequence that is at least 80% identical to SEQ ID NOs: 22, and (v) an optional NLS.

In some embodiments, the first genomic editor comprises, from N to C terminus, (i) an optional NLS, (ii) a D10A SpyCas9 nickase or a D16A Nme2Cas9 nickase, (iii) a linker comprising one or more sequences selected from SEQ ID NOs: 25-38, 39 and 72-133, (iv) cytidine deaminase comprising an amino acid sequence that is at least 80% identical to SEQ ID NOs: 22, and (v) an optional NLS.

In some embodiments, the first genomic editor comprises, from N to C terminus, (i) an optional NLS, (ii) a cytidine deaminase comprising an amino acid sequence that is at least 80% identical to SEQ ID NOs: 22; (iii) a linker comprising one or more sequences selected from SEQ ID NOs: 25-38, 39 and 72-133, (iv) a D10A SpyCas9 nickase or a D16A Nme2Cas9 nickase, and (v) an optional NLS.

In some embodiments, the first genomic editor comprises, from N to C terminus, (i) an optional NLS, (ii) a D10A SpyCas9 nickase or a D16A Nme2Cas9 nickase, (iii) a linker comprising one or more sequences selected from SEQ ID NOs: 25-38, 39 and 72-133, (iv) a cytidine deaminase comprising an amino acid sequence that is at least 80% identical to SEQ ID NOs: 22, and (v) an optional NLS.

In some embodiments, the first genomic editor comprises, from N to C terminus, (i) an optional NLS, (ii) a D10A SpyCas9 nickase or a D16A Nme2Cas9 nickase, (iii) a linker comprising one or more sequences selected from SEQ ID NOs: 25-38, 39 and 72-133, (iv) a cytidine deaminase comprising an amino acid sequence that is at least 80% identical to SEQ ID NOs: 22, and (v) an optional NLS.

In some embodiments, the first genomic editor comprises, from N to C terminus, (i) an optional NLS, (ii) a D10A SpyCas9 nickase or a D16A Nme2Cas9 nickase, (iii) a linker comprising one or more sequences selected from SEQ ID NOs: 25-38, 39 and 72-133, and (iv) cytidine deaminase comprising an amino acid sequence that is at least 80% identical to SEQ ID NOs: 22, and (v) an optional NLS.

### 2. Compositions Comprising an APOBEC3A Deaminase and an RNA-Guided Nickase

In some embodiments, a first genome editing tool comprising a first genomic editor is provided. In some embodiments, the first genomic editor comprises a base editor. In some embodiments, the first genomic editor or the base editor comprises a human A3A and an RNA-guided nickase. In some embodiments, the first genomic editor or the base editor comprises a wild-type A3A and an RNA-guided nickase. In some embodiments, the first genomic editor or the base editor comprises an A3A variant and an RNA-guided nickase. In some embodiments, the first genomic editor or the base editor comprises an A3A and a Cas9 nickase. In some embodiments, the first genomic editor or the base editor comprises an A3A and a D10A SpyCas9 nickase. In some embodiments, the first genomic editor or the base editor comprises a human A3A and a D10A SpyCas9 nickase. In some embodiments, the first genomic editor or the base editor comprises an A3A variant and a D10A SpyCas9 nickase. In some embodiments, the first genomic editor or the base editor lacks a UGI. In some embodiments, the first genomic editor or the base editor comprises one or more UGIs. In some embodiments, the first genomic editor or the base editor comprises two UGIs. In some embodiments, the A3A and the RNA-guided nickase are linked via a linker. In some embodiments, the first genomic editor or the base editor further comprises one or more additional heterologous functional domains. In some embodiments, the first genomic editor or the base editor further comprises a nuclear localization sequence (NLS) (described herein) at the C-terminal of the polypeptide or the N-terminal of the polypeptide.

In some embodiments, the first genomic editor or the base editor comprises a human A3A and a D10A SpyCas9 nickase, wherein the human A3A and the D10A SpyCas9 nickase are fused via a linker. In some embodiments, the first genomic editor or the base editor comprises a human A3A and a D10A SpyCas9 nickase, and a nuclear localization sequence (NLS) at the C-terminus of the fused polypeptide. In some embodiments, the first genomic editor or the base editor comprises a human A3A and a D10A SpyCas9 nickase, and a NLS at the N-terminus of the fused polypeptide. In some embodiments, the first genomic editor or the base editor comprises a human A3A and a D10A SpyCas9 nickase, wherein the human A3A and the D10A SpyCas9 nickase are fused via a linker, and a NLS fused to the C-terminus of the D10A SpyCas9 nickase, optionally via a linker. In some embodiments, the first genomic editor or the base editor comprises a human A3A and a D10A SpyCas9 nickase, wherein the human A3A and the D10A SpyCas9 nickase are fused via a linker, and a NLS fused to the C-terminus of the D10A SpyCas9 nickase, optionally via a linker.

In some embodiments, the first genomic editor or the base editor comprises a human A3A and a D16A NmeCas9 nickase, wherein the human A3A and the D16A NmeCas9 nickase are fused via a linker. In some embodiments, the first genomic editor or the base editor comprises a human A3A and a D16A NmeCas9 nickase, and a nuclear localization sequence (NLS) at the C-terminus of the fused polypeptide. In some embodiments, the first genomic editor or the base editor comprises a human A3A and a D16A NmeCas9 nickase, and a NLS at the N-terminus of the fused polypeptide. In some embodiments, the first genomic editor or the base editor comprises a human A3A and a D16A NmeCas9 nickase, wherein the human A3A and the D16A NmeCas9 nickase are fused via a linker, and a NLS fused to the C-terminus of the D16A NmeCas9 nickase, optionally via a linker. In some embodiments, the first genomic editor or the base editor comprises a human A3A and a D16A NmeCas9 nickase, wherein the human A3A and the D16A NmeCas9 nickase are fused via a linker, and a NLS fused to the C-terminus of the D16A NmeCas9 nickase, optionally via a linker.

The first genomic editor or the base editor may be organized in any number of ways to form a single chain. The NLS can be N- or C-terminal, or both N- and C-terminals. and the A3A can be N- or C-terminal as compared the RNA-guided nickase. In some embodiments, the first genomic editor or the base editor comprises, from N to C terminus, an A3A, an optional linker, an RNA-guided nickase, and an optional NLS. In some first genomic editor or the base editor, the polypeptide comprises, from N to C terminus, an RNA-guided nickase, an optional linker, an A3A, and an optional NLS. In some first genomic editor or the base editor, the polypeptide comprises, from N to C terminus, an optional NLS, an RNA-guided nickase, an optional linker, and an A3A. In some embodiments, the first genomic editor or the base editor comprises, from N to C terminus, an optional NLS, an RNA-guided nickase, an optional linker, and an A3A, and an optional NLS.

In any of the foregoing embodiments, the first genomic editor or the base editor may comprise an amino acid sequence having at least 80% identity to SEQ ID NO: 3, 6, or 146. In some embodiments, any of the foregoing levels of identity is at least 85%, at least 90%, at least 95%, at least 98%, at least 99%, or 100%. In some embodiments, the first genomic editor or the base editor disclosed herein may comprise an amino acid sequence with at least 90% identity to SEQ ID NO: 3, 6, or 146. In some embodiments, the first genomic editor or the base editor disclosed herein may comprise an amino acid sequence with at least 95% identity to SEQ ID NO: 3, 6, or 146. In some embodiments, the first genomic editor or the base editor disclosed herein may comprise an amino acid sequence with at least 98% identity to SEQ ID NO: 3, 6, or 146. In some embodiments, the first genomic editor or the base editor disclosed herein may comprise an amino acid sequence with at least 99% identity to SEQ ID NO: 3, 6, or 146. In some embodiments, the first genomic editor or the base editor disclosed herein may comprise an amino acid sequence of SEQ ID NO: 3, 6, or 146.

In any of the foregoing embodiments, a nucleic acid or ORF encoding the first genomic editor or the base editor disclosed herein may comprise a nucleic acid sequence having at least 80% identity to SEQ ID NO: 2, 5, or 147. In some embodiments, any of the foregoing levels of identity is at least 85%, at least 90%, at least 95%, at least 98%, at least 99%, or 100%.

In any of the foregoing embodiments, a nucleic acid or ORF encoding the first genomic editor or the base editor disclosed herein may comprise a nucleic acid sequence having at least 80% identity to SEQ ID NO: 1 or 4. In some embodiments, any of the foregoing levels of identity is at least 85%, at least 90%, at least 95%, at least 98%, at least 99%, or 100%.

In any of the foregoing embodiments, the first genomic editor or the base editor may comprise an amino acid sequence having at least 80% identity to any one of SEQ ID NOs: 9, 12, 18, and 21. In some embodiments, any of the foregoing levels of identity is at least 85%, at least 90%, at least 95%, at least 98%, at least 99%, or 100%. In some embodiments, the first genomic editor or the base editor disclosed herein may comprise an amino acid sequence with at least 90% identity to any one of SEQ ID NOs: 9, 12, 18, and 21. In some embodiments, the first genomic editor or the base editor disclosed herein may comprise an amino acid sequence with at least 95% identity to any one of SEQ ID NOs: 9, 12, 18, and 21. In some embodiments, the first genomic editor or the base editor disclosed herein may comprise an amino acid sequence with at least 98% identity to any one of SEQ ID NOs: 9, 12, 18, and 21. In some embodiments, the first genomic editor or the base editor disclosed herein may comprise an amino acid sequence with at least 99% identity to any one of SEQ ID NOs: 9, 12, 18, and 21. In some embodiments, the first genomic editor or the base editor disclosed herein may comprise an amino acid sequence of any one of SEQ ID NOs: 9, 12, 18, and 21.

In any of the foregoing embodiments, a nucleic acid or ORF encoding the first genomic editor or the base editor disclosed herein may comprise a nucleic acid sequence having at least 80% identity to any one of SEQ ID NOs: 8, 11, 17, and 20. In some embodiments, any of the foregoing levels of identity is at least 85%, at least 90%, at least 95%, at least 98%, at least 99%, or 100%.

In any of the foregoing embodiments, a nucleic acid or ORF encoding the first genomic editor or the base editor disclosed herein may comprise a nucleic acid sequence having at least 80% identity to any one of SEQ ID NOs: 7, 10, 16, and 19. In some embodiments, any of the foregoing levels of identity is at least 85%, at least 90%, at least 95%, at least 98%, at least 99%, or 100%.

In any of the foregoing embodiments, the first genomic editor or the base editor may comprise an amino acid sequence having at least 80%, at least 90%, at least 95%, at least 98%, at least 99%, or 100% identity to SEQ ID NO: 136, 139, 142, or 145. In some embodiments, the first genomic editor or the base editor disclosed herein may comprise an amino acid sequence of SEQ ID NO: 136, 139, 142, or 145. In any of the foregoing embodiments, a nucleic acid or ORF encoding the first genomic editor or the base editor disclosed herein may comprise a nucleic acid sequence having at least 80%, at least 90%, at least 95%, at least 98%, at least 99%, or 100% identity to SEQ ID NOs: SEQ ID NO: 135, 138, 141, or 144. In some embodiments, a nucleic acid or ORF encoding the first genomic editor or the base editor disclosed herein comprises a nucleic acid sequence of SEQ ID NOs: SEQ ID NO: 135, 138, 141, or 144. In any of the foregoing embodiments, a nucleic acid or ORF encoding the first genomic editor or the base editor disclosed herein may comprise a nucleic acid sequence having at least 80%, at least 90%, at least 95%, at least 98%, at least 99%, or 100% identity to SEQ ID NO: 134, 137, 140, or 143. In any of the foregoing embodiments, a nucleic acid or ORF encoding the first genomic editor or the base editor disclosed herein may comprise a nucleic acid sequence of SEQ ID NO: 134, 137, 140, or 143.

In any of the foregoing embodiments, the A3A may comprise an amino acid sequence having at least 80% identity to SEQ ID NO: 22. In some embodiments, the level of identity is at least 85%, at least 87%, at least 90%, at least 95%, at least 98%, at least 99%, or 100%. In some embodiments, the A3A comprises an amino acid sequence of SEQ ID NO: 22.

In any of the foregoing embodiments, the RNA-guided nickase may comprise an amino acid sequence having at least 80%, 90%, 95%, 98%, or 99% identity to any one of SEQ ID NO: 41, 43, or 45. In some embodiments, the level of identity is at least 85%, at least 87%, at least 90%, at least 95%, at least 98%, at least 99%, or 100%. In some embodiments, the RNA-guided nickase comprises the amino acid sequence of SEQ ID NO: 41. In some embodiments, the RNA-guided nickase comprises the amino acid sequence of SEQ ID NO: 43. In some embodiments, the RNA-guided nickase comprises the amino acid sequence of SEQ ID NO: 45.

In any of the foregoing embodiments, the A3A may comprise an amino acid sequence having at least 80% identity to SEQ ID NO: 22 and the RNA-guided nickase may comprise an amino acid sequence having at least 80%, 90%, 95%, 98%, or 99% identity to any one of SEQ ID NO: 41, 43, or 45. In some embodiments, the A3A comprises an amino acid sequence of SEQ ID NO: 22 and the RNA-guided nickase comprises an amino acid sequence of SEQ ID NO: 41.

In any of the foregoing embodiments, the a nucleic acid of ORF encoding the first genomic editor or the base editor comprises a nucleotide sequence that is at least 80%, 85%, 90%, 95%, 98%, or 100% identical to SEQ ID NO: 1. In any of the foregoing embodiments, a nucleic acid of ORF encoding the first genomic editor or the base editor comprises a nucleotide sequence that is at least 80%, 85%, 90%, 95%, 98%, or 100% identical to SEQ ID NO: 147. In any of the foregoing embodiments, a nucleic acid of ORF encoding the first genomic editor or the base editor comprises a nucleotide sequence that is at least 80%, 85%, 90%, 95%, 98%, or 100% identical to SEQ ID NO: 310.

### III. SECOND GENOME EDITING TOOL

In some embodiments, the second genome editing tool comprises a second genomic editor and at least one gRNA that targets at least one genomic locus and that is cognate to the second genomic editor, wherein the first genomic editor is orthogonal to the second genomic editor. In some embodiments, the second genome editing tool comprises a second genomic editor comprising an RNA-guided cleavase, and at least one gRNA that targets at least one genomic locus and that is cognate to the RNA-guided cleavase, wherein the base editor is orthogonal to the RNA-guided cleavase.

In some embodiments, the second genomic editor is delivered to the cell as at least one polypeptide or at least one mRNA. In some embodiments, the second genomic editor comprises at least one polypeptide or at least one mRNA. In some embodiments, the second genomic editor comprises a cleavase, a nickase, a catalytically inactive nuclease, a base editor, optionally a C to T base editor or an A to G base editor, or a fusion protein comprising a DNA polymerase and a nickase.

In some embodiments, one of the first genomic editor and the second genomic editor comprises a base editor, optionally a C to T base editor or an A to G base editor, and the other of the first genomic editor and the second genomic editor comprises a cleavase. In some embodiments, one of the first genomic editor and the second genomic editor comprises a C to T base editor, and the other of the first genomic editor and the second genomic editor comprises an A to G base editor. In some embodiments, one of the first genomic editor and second genomic editor comprises an N. meningitidis (Nine) RNA-guided nickase or cleavase, and the other of the first genomic editor and the second genomic editor comprises an S. pyogenes (Spy) RNA-guided nickase or cleavase.

In some embodiments, the second genomic editor or the RNA-guided cleavase is a Cas nuclease. In some embodiments, the Cas nuclease is a Cas9. In some embodiments, the Cas9 is Streptococcus pyogenes Cas9 (SpyCas9), S. aureus Cas9 (SauCas9), C. diphtheriae Cas9 (CdiCas9), Streptococcus thermophilus Cas9 (St1Cas9), A. cellulolyticus Cas9 (AceCas9), C. jejuni Cas9 (CjeCas9). R. palustris Cas9 (RpaCas9), R. rubrum Cas9 (RruCas9), A. naeslundii Cas9 (AnaCas9), Francisella novicida Cas9 (FnoCas9), or N. meningitidis (NmeCas9). In some embodiments, the Cas9 is an Nme1Cas9, an Nme2Cas9, an Nme3Cas9, or SpyCas9. In some embodiments, the Cas nuclease is a Class 2 Cas nuclease. In some embodiments, the Cas nuclease is a Cas12. In some embodiments, the Cas12 is Lachnospiraceae bacterium Cas12a (LbCas12a) or the Cas12 is Acidaminococcus sp. Cas12a (AsCas12a). In some embodiments, the Cas nuclease is an Eubacterium siraeum Cas13d (EsCas13d).

In some embodiments, the second genomic editor or the RNA-guided cleavase is a Cas9 cleavase. In some embodiments, the second genomic editor or the RNA-guided cleavase is Streptococcus pyogenes Cas9 (SpyCas9) cleavase. In some embodiments, the SpyCas9 cleavase comprises an amino acid sequence that is at least 90% identical to SEQ ID NO: 156. In some embodiments, the SpyCas9 cleavase comprises the amino acid sequence of SEQ ID NO: 156.

In some embodiments, the second genomic editor or the RNA-guided cleavase is a Cas9 cleavase. In some embodiments, the second genomic editor or the RNA-guided cleavase is N. meningitidis Cas9 (NmeCas9) cleavase. In some embodiments, the NmeCas9 cleavase comprises an amino acid sequence that is at least 90%, 91%, 92%, 93%, 94%, 95%, 96%, 97%, 98%, 99%, or 100% identical to any one of SEQ ID NOs: 157, 158-167, 191, 198, 205, 212, and 219. In some embodiments, the NmeCas9 cleavase comprises the amino acid sequence of any one of SEQ ID NOs: 157, 158-167, 191, 198, 205, 212, and 219.

In some embodiments, the second genome editing tool, the nucleic acid encoding the RNA-guided cleavase, the second nucleic acid comprising the second ORF, or the second ORF comprises a polynucleotide sequence that is at least 90%, 91%, 92%, 93%, 94%, 95%, 96%, 97%, 98%, 99%, or 100% identical to any one of SEQ ID NOs: 168, 169-178, 180, 181-190, 192-197, 199-204, 206-211, 213-218, and 220-225. In some embodiments, the second genome editing tool, the nucleic acid encoding the RNA-guided cleavase, the second nucleic acid comprising the second ORF, or the second ORF comprises the polynucleotide sequence of any one of SEQ ID NOs: 168, 169-178, 180, 181-190, 192-197, 199-204, 206-211, 213-218, and 220-225.

In some embodiments, the second genome editing tool comprises an RNA-guided cleavase. In some embodiments, the RNA-guided cleavase, when used with the at least one gRNA cognate to the cleavase, provides for simultaneous knock-out of the genomic locus targeted by the at least one gRNA and knock-in of an exogeneous gene.

In some embodiments, the second genome editing tool comprises a fusion protein comprising a DNA polymerase and a nickase. In some embodiments, the fusion protein comprising a DNA polymerase and a nickase, when used with the at least one gRNA cognate to the nickase, provides for targeted knock-in of an exogeneous nucleic acid.

In some embodiments, the second genome editing tool may be combined with any first genome editing tool disclosed herein. In some embodiments, the second nucleic acid comprising any second ORF may be combined with any first nucleic acid comprising any first ORF disclosed herein. Use of a Cas9 nickase and a Cas9 cleavase that are orthologous to each other in the first genome editing tool and the second genome editing tool may prevent cross-utilization.

In some embodiments, the first genome editing tool comprises a first genomic editor or a base editor comprising a deaminase (e.g., a cytidine deaminase) of the APOBEC family and a D16A NmeCas9 nickase, and at least one gRNA that targets at least one genomic locus and that is cognate to the nickase. In some embodiments, the first genomic editor or the base editor comprises one or more UGIs. In some embodiments, the second genome editing tool comprises an S. pyogenes Cas9 (SpyCas9) cleavase, and at least one gRNA that targets at least one genomic locus and that is cognate to the SpyCas9 cleavase.

In some embodiments, the first genome editing tool comprises a first genomic editor or a base editor comprising a deaminase (e.g., a cytidine deaminase) of the APOBEC family and a D16A NmeCas9 nickase, and at least one gRNA that targets at least one genomic locus and that is cognate to the nickase. In some embodiments, the first genomic editor or the base editor does not comprise any UGIs. In some embodiments, the first genome editing tool further comprises at least one UGI in a polypeptide different from the first genomic editor or the base editor. In some embodiments, the second genome editing tool comprises an S. pyogenes Cas9 (SpyCas9) cleavase, and at least one gRNA that targets at least one genomic locus and that is cognate to the SpyCas9cleavase.

In some embodiments, the first genome editing tool comprises a first genomic editor or a base editor comprising a deaminase (e.g., a cytidine deaminase) of the APOBEC family and a D10A SpyCas9 nickase, and at least one gRNA that targets at least one genomic locus and that is cognate to the nickase. In some embodiments, the first genomic editor or the base editor comprises one or more UGIs. In some embodiments, the second genome editing tool comprises an NmeCas9 cleavase, and at least one gRNA that targets at least one genomic locus and that is cognate to the NmeCas9 cleavase.

In some embodiments, the first genome editing tool comprises a first genomic editor or a base editor comprising a deaminase (e.g., a cytidine deaminase) of the APOBEC family and a D10A SpyCas9 nickase, and at least one gRNA that targets at least one genomic locus and that is cognate to the nickase. In some embodiments, the first genomic editor or the base editor does not comprise any UGIs. In some embodiments, the first genome editing tool further comprises at least one UGI in a polypeptide different from the first genomic editor or the base editor. In some embodiments, the second genome editing tool comprises an NmeCas9 cleavase, and at least one gRNA that targets at least one genomic locus and that is cognate to the NmeCas9 cleavase.

### IV. ADDITIONAL FEATURES

The following section provides additional features of the first genomic editor, the base editor, the second genomic editor, and the nucleic acid encoding the same. In any of the embodiments set forth herein, the nucleic acid may be an expression construct comprising a promoter operably linked to an ORF encoding the first genomic editor, the base editor, or the second genomic editor disclosed herein.

### A. Codon-optimization

In some embodiments, the nucleic acid encoding the first genomic editor, the base editor, or the second genomic editor comprises an ORF comprising a codon optimized nucleic acid sequence. In some embodiment, the codon optimized nucleic acid sequence comprises minimal adenine codons and/or minimal uridine codons.

A given ORF can be reduced in uridine content or uridine dinucleotide content, for example, by using minimal uridine codons in a sufficient fraction of the ORF. For example, an amino acid sequence for the first genomic editor, the base editor, or the second genomic editor described herein can be back-translated into an ORF sequence by converting amino acids to codons, wherein some or all of the ORF uses the exemplary minimal uridine codons shown below. In some embodiments, at least about 50%, 55%, 60%, 65%, 70%, 75%, 80%, 85%, 90%, 95%, 98%, 99%, or 100% of the codons in the ORF are codons listed in Table 1.

TABLE 1
Exemplary minimal uridine codons

| | Amino Acid | Minimal uridine codon |
|---|---|---|
| A | Alanine | GCA or GCC or GCG |
| G | Glycine | GGA or GGC or GGG |
| V | Valine | GUC or GUA or GUG |
| D | Aspartic acid | GAC |
| E | Glutamic acid | GAA or GAG |
| I | Isoleucine | AUC or AUA |
| T | Threonine | ACA or ACC or ACG |
| N | Asparagine | AAC |
| K | Lysine | AAG or AAA |
| S | Serine | AGC |
| R | Arginine | AGA or AGG |
| L | Leucine | CUG or CUA or CUC |
| P | Proline | CCG or CCA or CCC |
| H | Histidine | CAC |
| Q | Glutamine | CAG or CAA |
| F | Phenylalanine | UUC |
| Y | Tyrosine | UAC |
| C | Cysteine | UGC |
| W | Tryptophan | UGG |
| M | Methionine | AUG |

In some embodiments, the ORF may consist of a set of codons of which at least about 75%, 80%, 85%, 90%, 95%, 98%, 99%, or 100% of the codons are codons listed in Table 1.

A given ORF can be reduced in adenine content or adenine dinucleotide content, for example, by using minimal adenine codons in a sufficient fraction of the ORF. For example, an amino acid sequence for the first genomic editor, the base editor, or the second genomic editor described herein can be back-translated into an ORF sequence by converting amino acids to codons, wherein some or all of the ORF uses the exemplary minimal adenine codons shown below. In some embodiments, at least about 50%, 55%, 60%, 65%, 70%, 75%, 80%, 85%, 90%, 95%, 98%, 99%, or 100% of the codons in the ORF are codons listed in Table 2.

TABLE 2
Exemplary minimal adenine codons

| | Amino Acid | Minimal adenine codon |
|---|---|---|
| A | Alanine | GCU or GCC or GCG |
| G | Glycine | GGU or GGC or GGG |
| V | Valine | GUC or GUU or GUG |
| D | Aspartic acid | GAC or GAU |
| E | Glutamic acid | GAG |
| I | Isoleucine | AUC or AUU |
| T | Threonine | ACU or ACC or ACG |
| N | Asparagine | AAC or AAU |
| K | Lysine | AAG |
| S | Serine | UCU or UCC or UCG |
| R | Arginine | CGU or CGC or CGG |
| L | Leucine | CUG or CUC or CUU |
| P | Proline | CCG or CCU or CCC |
| H | Histidine | CAC or CAU |
| Q | Glutamine | CAG |
| F | Phenylalanine | UUC or UUU |
| Y | Tyrosine | UAC or UAU |
| C | Cysteine | UGC or UGU |
| W | Tryptophan | UGG |
| M | Methionine | AUG |

In some embodiments, the ORF may consist of a set of codons of which at least about 75%, 80%, 85%, 90%, 95%, 98%, 99%, or 100% of the codons are codons listed in Table 2.

To the extent feasible, any of the features described above with respect to low adenine content can be combined with any of the features described above with respect to low uridine content. So too for uridine and adenine dinucleotides. Similarly, the content of uridine nucleotides and adenine dinucleotides in the ORF may be as set forth above. Similarly, the content of uridine dinucleotides and adenine nucleotides in the ORF may be as set forth above.

A given ORF can be reduced in uridine and adenine nucleotide or dinucleotide content, for example, by using minimal uridine and adenine codons in a sufficient fraction of the ORF. For example, an amino acid sequence for the polypeptide, the second genomic editor, or the RNA-guided cleavase described herein can be back-translated into an ORF sequence by converting amino acids to codons, wherein some or all of the ORF uses the exemplary minimal uridine and adenine codons shown below. In some embodiments, at least about 50%, 55%, 60%, 65%, 70%, 75%, 80%, 85%, 90%, 95%, 98%, 99%, or 100% of the codons in the ORF are codons listed in Table 3.

TABLE 3
Exemplary minimal uridine and adenine codons

| | | Minimal uridine and adenine |
|---|---|---|
| | Amino Acid | codon |
| A | Alanine | GCC or GCG |
| G | Glycine | GGC or GGG |
| V | Valine | GUC or GUG |
| D | Aspartic acid | GAC |
| E | Glutamic acid | GAG |
| I | Isoleucine | AUC |
| T | Threonine | ACC or ACG |
| N | Asparagine | AAC |
| K | Lysine | AAG |
| S | Serine | AGC or UCC or UCG |
| R | Arginine | CGC or CGG |
| L | Leucine | CUG or CUC |
| P | Proline | CCG or CCC |
| H | Histidine | CAC |
| Q | Glutamine | CAG |
| F | Phenylalanine | UUC |
| Y | Tyrosine | UAC |
| C | Cysteine | UGC |
| W | Tryptophan | UGG |
| M | Methionine | AUG |

In some embodiments, the ORF may consist of a set of codons of which at least about 75%, 80%, 85%, 90%, 95%, 98%, 99%, or 100% of the codons are codons listed in Table 3. As can be seen in Table 3, each of the three listed serine codons contains either one A or one U. In some embodiments, uridine minimization is prioritized by using AGC codons for serine. In some embodiments, adenine minimization is prioritized by using UCC or UCG codons for serine.

In some embodiments, the ORF may have codons that increase translation in a mammal, such as a human. In further embodiments, ORF is an mRNA and comprises codons that increase translation in an organ, such as the liver, of the mammal, e.g., a human. In further embodiments, the ORF may have codons that increase translation in a cell type, such as a hepatocyte, of the mammal, e.g., a human. An increase in translation in a mammal, cell type, organ of a mammal, human, organ of a human, etc., can be determined relative to the extent of translation wild-type sequence of the ORF, or relative to an ORF having a codon distribution matching the codon distribution of the organism from which the ORF was derived or the organism that contains the most similar ORF at the amino acid level. Alternatively, in some embodiments, an increase in translation for a Cas9 sequence in a mammal, cell type, organ of a mammal, human, organ of a human, etc., is determined relative to translation of an ORF with the sequence of SEQ ID NO: 2 or 5 with all else equal, including any applicable point mutations, heterologous domains, and the like. In some embodiments, at least about 75%, 80%, 85%, 90%, 95%, 96%, 97%, 98%, 99%, or 100% of the codons in an ORF are codons corresponding to highly expressed tRNAs (e.g., the highest-expressed tRNA for each amino acid) in a mammal, such as a human. In some embodiments, at least 75%, 80%, 85%, 90%, 95%, 96%, 97%, 98%, 99%, or 100% of the codons in an ORF are codons corresponding to highly expressed tRNAs (e.g., the highest-expressed tRNA for each amino acid) in a mammalian organ, such as a human organ.

Alternatively, codons corresponding to highly expressed tRNAs in an organism (e.g., human) in general may be used.

Any of the foregoing approaches to codon selection can be combined with the minimal uridine or adenine codons shown above, e.g., by starting with the codons of Table 1, Table 2, or Table 3, and then where more than one option is available, using the codon that corresponds to a more highly-expressed tRNA, either in the organism (e.g., human) in general, or in an organ or cell type of interest (e.g., human liver or human hepatocytes).

In some embodiments, at least 75%, 80%, 85%, 90%, 95%, 96%, 97%, 98%, 99%, or 100% of the codons in an ORF are codons from a codon set shown in Table 4 (e.g., the low U 1, low A, or low A/U codon set). The codons in the low U 1, low G, low A, and low A/U sets use codons that minimize the indicated nucleotides while also using codons corresponding to highly expressed tRNAs where more than one option is available. In some embodiments, at least 75%, 80%, 85%, 90%, 95%, 96%, 97%, 98%, 99%, or 100% of the codons in an ORF are codons from the low U 1 codon set shown in Table 4. In some embodiments, at least 75%, 80%, 85%, 90%, 95%, 96%, 97%, 98%, 99%, or 100% of the codons in an ORF are codons from the low A codon set shown in Table 4. In some embodiments, at least 75%, 80%, 85%, 90%, 95%, 96%, 97%, 98%, 99%, or 100% of the codons in an ORF are codons from the low A/U codon set shown in Table 4.

TABLE 4
Exemplary Codon Sets.

| Amino Acid | Low U 1 | Low U 2 | Low A | Low A/U |
|---|---|---|---|---|
| Gly | GGC | GGG | GGC | GGC |
| Glu | GAG | GAA | GAG | GAG |
| Asp | GAC | GAC | GAC | GAC |
| Val | GTG | GTA | GTG | GTG |
| Ala | GCC | GCG | GCC | GCC |
| Arg | AGA | CGA | CGG | CGG |
| Ser | AGC | AGC | TCC | AGC |
| Lys | AAG | AAA | AAG | AAG |
| Asn | AAC | AAC | AAC | AAC |
| Met | ATG | ATG | ATG | ATG |
| Ile | ATC | ATA | ATC | ATC |
| Thr | ACC | ACG | ACC | ACC |
| Trp | TGG | TGG | TGG | TGG |
| Cys | TGC | TGC | TGC | TGC |
| Tyr | TAC | TAC | TAC | TAC |
| Leu | CTG | CTA | CTG | CTG |
| Phe | TTC | TTC | TTC | TTC |
| Gln | CAG | CAA | CAG | CAG |
| His | CAC | CAC | CAC | CAC |

### B. Heterologous Functional Domains; Nuclear Localization Signals (NLS)

In some embodiments, the first genomic editor, the base editor, or the second genomic editor disclosed herein further comprises one or more additional heterologous functional domains (e.g., is or comprises a ternary or higher-order fusion polypeptide).

In some embodiments, the heterologous functional domain may facilitate transport of the first genomic editor, the base editor, or the second genomic editor into the nucleus of a cell. For example, the heterologous functional domain may be a nuclear localization signal (NLS). In some embodiments, the first genomic editor, the base editor, or the second genomic editor may be fused with 1-10 NLS(s). In some embodiments, the first genomic editor, the base editor, or the second genomic editor may be fused with 1-5 NLS(s). In some embodiments, the first genomic editor, the base editor, or the second genomic editor may be fused with one NLS. Where one NLS is used, the NLS may be fused at the N-terminus or the C-terminus of first genomic editor, the base editor, or the second genomic editor sequence. In some embodiments, the first genomic editor, the base editor, or the second genomic editor may be fused C-terminally to at least one NLS. An NLS may also be inserted within the polypeptide, the second genomic editor, or the RNA-guided cleavase sequence. In other embodiments, the first genomic editor, the base editor, or the second genomic editor may be fused with more than one NLS. In some embodiments, the first genomic editor, the base editor, or the second genomic editor may be fused with 2, 3, 4, or 5 NLSs. In some embodiments, the first genomic editor, the base editor, or the second genomic editor may be fused with two NLSs. In certain circumstances, the two NLSs may be the same (e.g., two SV40 NLSs) or different. In some embodiments, the first genomic editor, the base editor, or the second genomic editor is fused to two SV40 NLS sequences at the carboxy terminus. In some embodiments, the first genomic editor, the base editor, or the second genomic editor may be fused with two NLSs, one at the N-terminus and one at the C-terminus. In some embodiments, the first genomic editor, the base editor, or the second genomic editor may be fused with 3 NLSs. In some embodiments, the first genomic editor, the base editor, or the second genomic editor may be fused with no NLS. In some embodiments, the NLS may be a monopartite sequence, such as, e.g., the SV40 NLS, PKKKRKV (SEQ ID NO: 40) or PKKKRRV (SEQ ID NO: 70). In some embodiments, the NLS may be a bipartite sequence, such as the NLS of nucleoplasmin, KRPAATKKAGQAKKKK (SEQ ID NO: 71). In a specific embodiment, a single PKKKRKV (SEQ ID NO: 40) NLS may be fused at the C-terminus of the first genomic editor, the base editor, or the second genomic editor. One or more linkers are optionally included at the fusion site (e.g., between the first genomic editor, the base editor, or the second genomic editor and NLS). In some embodiments, one or more NLS(s) according to any of the foregoing embodiments are present in the first genomic editor, the base editor, or the second genomic editor in combination with one or more additional heterologous functional domains, such as any of the heterologous functional domains described below.

In some embodiments, the cytidine deaminase (e.g., A3A) is located N-terminal to the RNA-guided nickase in the first genomic editor or the base editor. In some embodiments, the RNA-guided nickase comprises a nuclear localization signal (NLS). In some embodiments, the NLS is fused to the C-terminus of the RNA-guided nickase. In some embodiments, the NLS is fused to the C-terminus of the RNA-guided nickase via a linker. In some embodiments, the NLS is fused to the N-terminus of the RNA-guided nickase. In some embodiments, the NLS is fused to the N-terminus of the RNA-guided nickase via a linker (e.g., SEQ ID NO: 39). In some embodiments, the NLS comprises a sequence having at least 80%, 85%, 90%, or 95% identity to any one of SEQ ID NOs: 40 and 59-71. In some embodiments, the NLS comprises the sequence of any one of SEQ ID NOs: 40 and 59-71. In some embodiments, the NLS is encoded by a sequence having at least 80%, 85%, 90%, 95%, 98% or 100% identity to the sequence of any one of SEQ ID NOs: 40 and 59-71.

In some embodiments, the heterologous functional domain may be capable of modifying the intracellular half-life of the A3A or the RNA-guided nickase in the first genomic editor or the base editor. In some embodiments, the half-life of the A3A or the RNA-guided nickase in the polypeptide may be increased. In some embodiments, the half-life of the A3A or the RNA-guided nickase in the first genomic editor or the base editor may be reduced. In some embodiments, the heterologous functional domain may be capable of increasing the stability of the A3A or the RNA-guided nickase in the first genomic editor or the base editor. In some embodiments, the heterologous functional domain may be capable of reducing the stability of the A3A or the RNA-guided nickase in the first genomic editor or the base editor. In some embodiments, the heterologous functional domain may act as a signal peptide for protein degradation. In some embodiments, the protein degradation may be mediated by proteolytic enzymes, such as, for example, proteasomes, lysosomal proteases, or calpain proteases. In some embodiments, the heterologous functional domain may comprise a PEST sequence. In some embodiments, the polypeptide may be modified by addition of ubiquitin or a polyubiquitin chain. In some embodiments, the ubiquitin may be a ubiquitin-like protein (UBL). Non-limiting examples of ubiquitin-like proteins include small ubiquitin-like modifier (SUMO), ubiquitin cross-reactive protein (UCRP, also known as interferon-stimulated gene-15 (ISG15)), ubiquitin-related modifier-1 (URM1), neuronal-precursor-cell-expressed developmentally downregulated protein-8 (NEDD8, also called Rubl in S. cerevisiae), human leukocyte antigen F-associated (FAT10), autophagy-8 (ATG8) and −12 (ATG12), Fau ubiquitin-like protein (FUB1), membrane-anchored UBL (MUB), ubiquitin fold-modifier-1 (UFM1), and ubiquitin-like protein-5 (UBL5).

In some embodiments, the heterologous functional domain may be a marker domain. Non-limiting examples of marker domains include fluorescent proteins, purification tags, epitope tags, and reporter gene sequences. In some embodiments, the marker domain may be a fluorescent protein. Any known fluorescent proteins may be used as the marker domain such as GFP, YFP, EBFP, ECFP, DsRed or any other suitable fluorescent protein. In some embodiments, the marker domain may be a purification tag or an epitope tag. Non-limiting exemplary tags include glutathione-S-transferase (GST), chitin binding protein (CBP), maltose binding protein (MBP), thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6xHis (SEQ ID NO: 401), 8xHis (SEQ ID NO: 402), biotin carboxyl carrier protein (BCCP), poly-His, and calmodulin. In some embodiments, the marker domain may be a reporter gene. Non-limiting exemplary reporter genes include glutathione-S-transferase (GST), horseradish peroxidase (HRP), chloramphenicol acetyltransferase (CAT), beta-galactosidase, beta-glucuronidase, luciferase, or fluorescent proteins.

In additional embodiments, the heterologous functional domain may target the first genomic editor, the base editor, or the second genomic editor to a specific organelle, cell type, tissue, or organ. In some embodiments, the heterologous functional domain may target the first genomic editor, the base editor, or the second genomic editor to mitochondria.

### C. UTRs; Kozak Sequences

In some embodiments, the nucleic acid (e.g., mRNA) disclosed herein comprises a 5′ UTR, 3′ UTR, or 5′ and 3′ UTRs from Hydroxysteroid 17-Beta Dehydrogenase 4 (HSD17B4 or HSD) or globin such as human alpha globin (HBA), human beta globin (HBB), Xenopus laevis beta globin (XBG), bovine growth hormone, cytomegalovirus (CMV), mouse Hba-al, heat shock protein 90 (Hsp90), glyceraldehyde 3-phosphate dehydrogenase (GAPDH), beta-actin, alpha-tubulin, tumor protein (p53), or epidermal growth factor receptor (EGFR).

In some embodiments, the nucleic acid described herein does not comprise a 5′ UTR, e.g., there are no additional nucleotides between the 5′ cap and the start codon. In some embodiments, the nucleic acid comprises a Kozak sequence (described below) between the 5′ cap and the start codon, but does not have any additional 5′ UTR. In some embodiments, the nucleic acid does not comprise a 3′ UTR, e.g., there are no additional nucleotides between the stop codon and the poly-A tail.

In some embodiments, the nucleic acid herein comprises a Kozak sequence. The Kozak sequence can affect translation initiation and the overall yield of a polypeptide translated from an mRNA. A Kozak sequence includes a methionine codon that can function as the start codon. A minimal Kozak sequence is NNNRUGN wherein at least one of the following is true: the first N is A or G and the second N is G. In the context of a nucleotide sequence, R means a purine (A or G). In some embodiments, the Kozak sequence is RNNRUGN, NNNRUGG, RNNRUGG, RNNAUGN, NNNAUGG, RNNAUGG, or GCCACCAUG.

### D. Poly-A Tail

In some embodiments, the nucleic acid disclosed herein further comprises a poly-adenylated (poly-A) tail. The poly-A tails may comprise at least 8 consecutive adenine nucleotides, but also comprise one or more non-adenine nucleotide. As used herein, “non-adenine nucleotides” refer to any natural or non-natural nucleotides that do not comprise adenine. Guanine, thymine, and cytosine nucleotides are exemplary non-adenine nucleotides. Thus, the poly-A tails on the nucleic acid described herein may comprise consecutive adenine nucleotides located 3′ to nucleotides encoding a polypeptide of interest. In some instances, the poly-A tails on the nucleic acid comprise non-consecutive adenine nucleotides located 3′ to nucleotides encoding the polypeptide, wherein non-adenine nucleotides interrupt the adenine nucleotides at regular or irregularly spaced intervals.

In some embodiments, the poly-A tail is encoded in a plasmid used for in vitro transcription of an mRNA and becomes part of the transcript. The poly-A sequence encoded in the plasmid, i.e., the number of consecutive adenine nucleotides in the poly-A sequence, may not be exact, e.g., a 100 poly-A sequence (SEQ ID NO: 403) in the plasmid may not result in a precisely 100 poly-A sequence (SEQ ID NO: 403) in the transcribed mRNA. In some embodiments, the poly-A tail is not encoded in the plasmid, and is added by PCR tailing or enzymatic tailing, e.g., using E. coli poly(A) polymerase.

In some embodiments, the one or more non-adenine nucleotides are positioned to interrupt the consecutive adenine nucleotides so that a poly(A) binding protein can bind to a stretch of consecutive adenine nucleotides. In some embodiments, one or more non-adenine nucleotide(s) is located after at least 8, 9, 10, 11, or 12 consecutive adenine nucleotides (SEQ ID NO: 404). In some embodiments, the one or more non-adenine nucleotide is located after 8-50 consecutive adenine nucleotides (SEQ ID NO: 405). In some embodiments, the one or more non-adenine nucleotide is located after 8-100 consecutive adenine nucleotides (SEQ ID NO: 406).

In some embodiments, the poly-A tail comprises or contains one non-adenine nucleotide or one consecutive stretch of 2-10 non-adenine nucleotides.

In some embodiments, the non-adenine nucleotide is guanine, cytosine, or thymine. In some instances, where more than one non-adenine nucleotide is present, the non-adenine nucleotide may be selected from: a) guanine and thymine nucleotides; b) guanine and cytosine nucleotides; c) thymine and cytosine nucleotides; or d) guanine, thymine and cytosine nucleotides.

### E. Modified Nucleotides

In some embodiments, the nucleic acid disclosed herein comprises a modified uridine at some or all uridine positions. In some embodiments, the modified uridine is a uridine modified at the 5 position, e.g., with a halogen or C1-C3 alkoxy. In some embodiments, the modified uridine is a pseudouridine modified at the 1 position, e.g., with a C1-C3 alkyl. The modified uridine can be, for example, pseudouridine, N1-methyl-pseudouridine, 5-methoxyuridine, 5-iodouridine, or a combination thereof.

In some embodiments, at least 10%, 15%, 20%, 25%, 30%, 35%, 40%, 45%, 50%, 55%, 60%, 65%, 70%, 75%, 80%, 85%, 90%, 95%, 98%, 99%, or 100% of the uridine positions in the nucleic acid disclosed herein are modified uridines. In some embodiments, 10%-25%, 15-25%, 25-35%, 35-45%, 45-55%, 55-65%, 65-75%, 75-85%, 85-95%, or 90-100% of the uridine positions in an mRNA disclosed herein are modified uridines, e.g., 5-methoxyuridine, 5-iodouridine, N1-methyl pseudouridine, pseudouridine, or a combination thereof.

In some embodiments, at least 10% of the uridine is substituted with a modified uridine. In some embodiments, 15% to 45% of the uridine is substituted with the modified uridine. In some embodiments, at least 20%, at least 30%, at least 40%, at least 50%, at least 60%, at least 70%, at least 80%, at least 90%, or 100% of the uridine is substituted with the modified uridine.

### F. 5′ Cap

In some embodiments, the nucleic acid disclosed herein comprises a 5′ cap, such as a Cap0, Cap1, or Cap2. A 5′ cap is generally a 7-methylguanine ribonucleotide (which may be further modified, as discussed below e.g., with respect to ARCA) linked through a 5′-triphosphate to the 5′ position of the first nucleotide of the 5′-to-3′ chain of the nucleic acid, i.e., the first cap-proximal nucleotide. In Cap0, the riboses of the first and second cap-proximal nucleotides of the mRNA both comprise a 2′-hydroxyl. In Cap1, the riboses of the first and second transcribed nucleotides of the nucleic acid comprise a 2′-methoxy and a 2′-hydroxyl, respectively. In Cap2, the riboses of the first and second cap-proximal nucleotides of the nucleic acid both comprise a 2′-methoxy. See, e.g., Katibah et al. (2014) Proc Natl Acad Sci USA 111(33):12025-30; Abbas et al. (2017) Proc Natl Acad Sci USA 114(11):E2106-E2115. Most endogenous higher eukaryotic nucleic acids, including mammalian nucleic acids such as human nucleic acids, comprise Cap1 or Cap2. Cap0 and other cap structures differing from Cap1 and Cap2 may be immunogenic in mammals, such as humans, due to recognition as “non-self” by components of the innate immune system such as IFIT-1 and IFIT-5, which can result in elevated cytokine levels including type I interferon. Components of the innate immune system such as IFIT-1 and IFIT-5 may also compete with eIF4E for binding of a nucleic acids with a cap other than Cap1 or Cap2, potentially inhibiting translation of the nucleic acid.

A cap can be included co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap or a Cap0-like cap in which the 2′ position of the first cap-proximal nucleotide is hydroxyl. See, e.g., Stepinski et al., (2001) “Synthesis and properties of mRNAs containing the novel ‘anti-reverse’ cap analogs 7-methyl(3′-O-methyl)GpppG and 7-methyl(3′deoxy)GpppG,” RNA 7: 1486-1495. The ARCA structure is shown below.

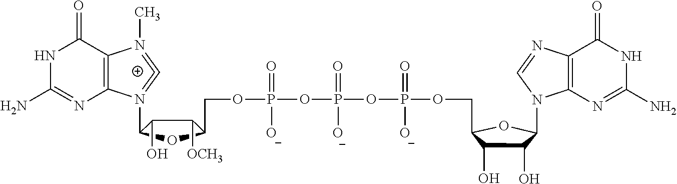

CleanCap™ AG (m7G(5′)ppp(5′)(2′OMeA)pG; TriLink Biotechnologies Cat. No. N7113) or CleanCap™ GG (m7G(5′)ppp(5′)(2′OMeG)pG; TriLink Biotechnologies Cat. No. N-7133) can be used to provide a Cap1 structure co-transcriptionally. 3′-O-methylated versions of CleanCap™ AG and CleanCap™ GG are also available from TriLink Biotechnologies as Cat. Nos. N-7413 and N-7433, respectively. The CleanCap™ AG structure is shown below. CleanCap™ structures are sometimes referred to herein using the last three digits of the catalog numbers listed above (e.g., “CleanCap™ 113” for TriLink Biotechnologies Cat. No. N-7113).

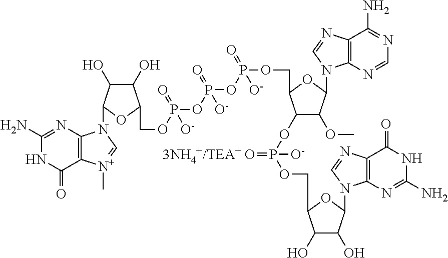 [SMILES: COC1C(OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(N)ncnc21]

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its D1 subunit, and guanine methyltransferase, provided by its D12 subunit. As such, it can add a 7-methylguanine to an RNA, so as to give Cap0, in the presence of S-adenosyl methionine and GTP. See, e.g., Guo, P. and Moss, B. (1990) Proc. Natl. Acad. Sci. USA 87, 4023-4027; Mao, X. and Shuman, S. (1994) J. Biol. Chem. 269, 24472-24479. For additional discussion of caps and capping approaches, see, e.g., WO2017/053297 and Ishikawa et al., Nucl. Acids. Symp. Ser. (2009) No. 53, 129-130.

### V. CELLS

In some embodiments, a cell contacted with the first genome editing tool or the second genome editing tool is a human cell.

In some embodiments, a cell is contacted with (a) a first genome editing tool, wherein the first genome editing tool comprises a first genomic editor and at least one guide RNA (gRNA) that targets at least one genomic locus and that is cognate to the first genomic editor; and (b) a second genome editing tool, wherein the second genome editing tool comprises a second genomic editor and at least one gRNA that targets at least one genomic locus and that is cognate to the second genomic editor, wherein the first genomic editor is orthogonal to the second genomic editor, thereby producing at least two genome edits in the cell.

In some embodiments, a cell is contacted with (a) with a first genome editing tool comprising a first genomic editor comprising a base editor, and at least one guide RNA (gRNA) that targets at least one genomic locus and that is cognate to the base editor; and (b) with a second genome editing tool comprising a second genomic editor comprising an RNA-guided cleavase, and at least one gRNA that targets at least one genomic locus and that is cognate to the RNA-guided cleavase, wherein the base editor is orthogonal to the RNA-guided cleavase, thereby producing at least two genome edits in the cell.

In some embodiments, a cell is contacted with (a) with a first genome editing tool comprising a first genomic editor comprising a base editor, and at least one guide RNA (gRNA) that targets at least one genomic locus and that is cognate to the base editor; and (b) with a second genome editing tool comprising a second genomic editor comprising an RNA-guided cleavase, and at least one gRNA that targets at least one genomic locus and that is cognate to the RNA-guided cleavase, wherein the base editor is orthogonal to the RNA-guided cleavase; in some embodiments, the cell is (c) cultured, thereby producing a population of cells comprising edited cells comprising at least two genome edits per cell.

In some embodiments, a cell is treated in vitro with any method or composition disclosed herein. In some embodiments, a cell is treated in vivo with any method or composition disclosed herein.

In some embodiments, the cell in any of the embodiments provided herein is engineered by a first genome editing tool and a second genome editing tool. In some embodiment, the first genome editing tool comprises a C to T base editor or an A to G base editor. In some embodiments, the first genome editing tool comprises a first genomic editor comprising a cytidine deaminase and an RNA-guided nickase, or a nucleic acid encoding the polypeptide. In some embodiments, the cytidine deaminase is APOBEC3A deaminase (A3A). In some embodiments, the first genomic editor comprises an amino acid sequence that is at least 80%, 85%, 90%, 95%, 98%, or 100% identical to SEQ ID NO: 3, SEQ ID NO: 146 or SEQ ID NO: 311. In some embodiments, the nucleic acid encoding the first genomic editor comprises a sequence that is at least 80%, 85%, 90%, 95%, 98%, or 100% identical to SEQ ID NO: 1, SEQ ID NO: 147, or SEQ ID NO: 310. In some embodiments, the first genomic editor comprises an amino acid sequence that is at least 80%, 85%, 90%, 95%, 98%, or 100% identical to any one of SEQ ID NOs: 9, 12, 18, and 21.

In some embodiments, the first genome editing tool or the second genome editing tool is delivered to the cell via electroporation. In some embodiments, the first genome editing tool or the second genome editing tool is delivered to the cell via at least one lipid nanoparticle (LNP). In some embodiments, the first genome editing tool or the second genome editing tool is contained in at least one LNP. In some embodiments, the first genome editing tool or the second genome editing tool is delivered to the cell on at least one vector. In some embodiments, the first genome editing tool or the second genome editing tool comprises at least one vector. In some embodiments, the first genome editing tool or the second genome editing tool is delivered as at least one nucleic acid encoding the first genome editing tool or the second genome editing tool. In some embodiments, the first genome editing tool or the second genome editing tool comprises at least one nucleic acid encoding the first genome editing tool or the second genome editing tool. In some embodiments, the first genome editing tool comprises at least one polypeptide comprising the first genome editing tool or at least one nucleic acid encoding the first genome editing tool. In some embodiments, the second genome editing tool comprises at least one polypeptide comprising the second genome editing tool or at least one nucleic acid encoding the second genome editing tool. In some embodiments, the at least one nucleic acid comprises at least one mRNA. In some embodiments, the first genomic editor or the second genomic editor is delivered to the cell as at least one polypeptide or at least one mRNA. In some embodiments, the first genomic editor or the second genomic editor comprises at least one polypeptide or at least one mRNA. In some embodiments, the at least one gRNA is delivered to the cell as at least one polynucleotide that encodes the gRNA. In some embodiments, the cell is contacted with a nucleic acid encoding an exogenous gene for insertion into a genomic locus. In some embodiments, the cell is contacted with a nucleic acid encoding an exogenous gene for insertion into the TRAC or AAVS1 locus

In some embodiments, in any of the methods disclosed herein, step (a) and step (b) of contacting the cell are performed simultaneously. In some embodiments, step (a) and step (b) of contacting the cell are performed in any order over a time period of about 5 minutes, 10 minutes, 15 minutes, 30 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 30 hours, 36 hours, or 48 hours. In some embodiments, each of step (a) and step (b) is independently performed over a time period of about 5 minutes, 10 minutes, 15 minutes, 30 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 30 hours, 36 hours, or 48 hours.

In some embodiments, the cell is an immune cell. As used herein, “immune cell” refers to a cell of the immune system, including e.g., a lymphocyte (e.g., T cell, B cell, natural killer cell (“NK cell”, and NKT cell, or iNKT cell)), monocyte, macrophage, mast cell, dendritic cell, or granulocyte (e.g., neutrophil, eosinophil, and basophil). In some embodiments, the cell is a primary immune cell. In some embodiments, the immune system cell may be selected from CD3⁺, CD4⁺ and CD8⁺ T cells, regulatory T cells (Tregs), B cells, NK cells, and dendritic cells (DC). In some embodiments, the immune cell is allogeneic.

In some embodiments, the cell is a lymphocyte. In some embodiments, the cell is an adaptive immune cell. In some embodiments, the cell is a T cell. In some embodiments, the cell is a B cell. In some embodiments, the cell is a NK cell.

As used herein, a T cell can be defined as a cell that expresses a T cell receptor (“TCR” or “αβ TCR” or “γδ TCR”), however in some embodiments, the TCR of a T cell may be genetically modified to reduce its expression (e.g., by genetic modification to the TRAC or TRBC genes), therefore expression of the protein CD3 may be used as a marker to identify a T cell by standard flow cytometry methods. CD3 is a multi-subunit signaling complex that associates with the TCR. Thus, a T cell may be referred to as CD3+. In some embodiments, a T cell is a cell that expresses a CD3+ marker and either a CD4+ or CD8+ marker.

In some embodiments, the T cell expresses the glycoprotein CD8 and therefore is CD8+ by standard flow cytometry methods and may be referred to as a “cytotoxic” T cell. In some embodiments, the T cell expresses the glycoprotein CD4 and therefore is CD4+ by standard flow cytometry methods and may be referred to as a “helper” T cell. CD4+ T cells can differentiate into subsets and may be referred to as a Th1 cell, Th2 cell, Th9 cell, Th17 cell, Th22 cell, T regulatory (“Treg”) cell, or T follicular helper cells (“Tfh”). Each CD4+ subset releases specific cytokines that can have either proinflammatory or anti-inflammatory functions, survival or protective functions. A T cell may be isolated from a subject by CD4+ or CD8+ selection methods.

In some embodiments, the T cell is a memory T cell. In the body, a memory T cell has encountered antigen. A memory T cell can be located in the secondary lymphoid organs (central memory T cells) or in recently infected tissue (effector memory T cells). A memory T cell may be a CD8+ T cell. A memory T cell may be a CD4+ T cell.

As used herein, a “central memory T cell” can be defined as an antigen-experienced T cell, and for example, may express CD62L and CD45RO. A central memory T cell may be detected as CD62L+ and CD45RO+ by central memory T cells also express CCR7, therefore may be detected as CCR7+ by standard flow cytometry methods.

As used herein, an “early stem-cell memory T cell” (or “Tscm”) can be defined as a T cell that expresses CD27 and CD45RA, and therefore is CD27+ and CD45RA+ by standard flow cytometry methods. A Tscm does not express the CD45 isoform CD45RO, therefore a Tscm will further be CD45RO− if stained for this isoform by standard flow cytometry methods. A CD45RO− CD27+ cell is therefore also an early stem-cell memory T cell. Tscm cells further express CD62L and CCR7, therefore may be detected as CD62L+ and CCR7+ by standard flow cytometry methods. Early stem-cell memory T cells have been shown to correlate with increased persistence and therapeutic efficacy of cell therapy products.

In some embodiments, the cell is a B cell. As used herein, a “B cell” can be defined as a cell that expresses CD19 or CD20, or B cell mature antigen (“BCMA”), and therefore a B cell is CD19+, or CD20+, or BCMA+ by standard flow cytometry methods. A B cell is further negative for CD3 and CD56 by standard flow cytometry methods. The B cell may be a plasma cell. The B cell may be a memory B cell. The B cell may be a naïve B cell. The B cell may be IgM+ or has a class-switched B cell receptor (e.g., IgG+, or IgA+).

In some embodiments, the cell is a mononuclear cell, such as from bone marrow or peripheral blood. In some embodiments, the cell is a peripheral blood mononuclear cell (“PBMC”). In some embodiments, the cell is a PBMC, e.g. a lymphocyte or monocyte. In some embodiments, the cell is a peripheral blood lymphocyte (“PBL”).

In some embodiments, the cell is derived from a progenitor cell before editing. In some embodiments, the cell is an induced pluripotent stem cell (iPSC).

Cells used in ACT therapy are included, such as mesenchymal stem cells (e.g., isolated from bone marrow (BM), peripheral blood (PB), placenta, umbilical cord (UC) or adipose); hematopoietic stem cells (HSCs; e.g. isolated from BM); mononuclear cells (e.g., isolated from BM or PB); endothelial progenitor cells (EPCs; isolated from BM, PB, and UC); neural stem cells (NSCs); limbal stem cells (LSCs); or tissue-specific primary cells or cells derived therefrom (TSCs). Cells used in ACT therapy further include induced pluripotent stem cells (iPSCs; see e.g., Mahla, International J. Cell Biol. 2016 (Article ID 6940283): 1-24 (2016)) that may be induced to differentiate into other cell types including e.g., islet cells, neurons, and blood cells; ocular stem cells; pluripotent stem cells (PSCs); embryonic stem cells (ESCs); cells for organ or tissue transplantations such as islet cells, cardiomyocytes, thyroid cells, thymocytes, neuronal cells, skin cells, retinal cells, chondrocytes, myocytes, and keratinocytes.

In some embodiments, the cell is a human cell, such as a cell from a subject. In some embodiments, the cell is isolated from a human subject. In some embodiments, the cell is isolated from a patient. In some embodiments, the cell is isolated from a donor. In some embodiments, the cell is isolated from human donor PBMCs or leukopaks. In some embodiments, the cell is from a subject with a condition, disorder, or disease. In some embodiments, the cell is from a human donor with Epstein Barr Virus (“EBV”).

In some embodiments, the cell is homozygous for HLA-B and homozygous for HLA-C. In some embodiments, the cell contains a genetic modification in the HLA-A gene and is homozygous for HLA-B and homozygous for HLA-C. In some embodiments, the cell is homozygous for HLA-A and homozygous for HLA-C. In some embodiments, the cell contains a genetic modification in the HLA-B gene and is homozygous for HLA-A and homozygous for HLA-C. In some embodiments, the cell is homozygous for HLA-C. In some embodiments, the cell contains a genetic modification in the HLA-A gene and a genetic modification in the HLA-B gene and is homozygous for HLA-C.

In some embodiments, the methods disclosed herein are carried out ex vivo. As used herein, “ex vivo” refers to an in vitro method wherein the cell is capable of being transferred into a subject, e.g. as an ACT therapy. In some embodiments, an ex vivo method is an in vitro method involving an ACT therapy cell or cell population.

In some embodiments, the cell is maintained in culture. In some embodiments, the cell is transplanted into a patient. In some embodiments, the cell is removed from a subject, genetically modified ex vivo, and then administered back to the same patient. In some embodiments, the cell is removed from a subject, genetically modified ex vivo, and then administered to a subject other than the subject from which it was removed.

In some embodiments, the cell is from a cell line. In some embodiments, the cell line is derived from a human subject. In some embodiments, the cell line is a lymphoblastoid cell line (“LCL”). The cell may be cryopreserved and thawed. The cell may not have been previously cryopreserved.

In some embodiments, the cell is from a cell bank. In some embodiments, the cell is genetically modified and then transferred into a cell bank. In some embodiments the cell is removed from a subject, genetically modified ex vivo, and transferred into a cell bank. In some embodiments, a genetically modified population of cells is transferred into a cell bank. In some embodiments, a genetically modified population of immune cells is transferred into a cell bank. In some embodiments, a genetically modified population of immune cells comprising a first and second subpopulations, wherein the first and second sub-populations have at least one common genetic modification and at least one different genetic modification are transferred into a cell bank.

In some embodiments, a population of cells comprises any cell edited using any method or composition disclosed herein.

In some embodiments, a population of cells comprises edited T cells, and wherein at least 30%, 40%, 50%, 55%, 60%, 65% of the cells of the population have a memory phenotype (CD27+, CD45RA+).

In some embodiments, a population of cells comprises non-activated immune cells. In some embodiments, the population of cells comprises activated immune cells.

In some embodiments, a population of cells comprises T cells and is responsive to repeat stimulation after editing. In some embodiments, the population of cells is cultured, expanded, differentiated, or proliferated ex vivo.

### VI. GUIDE RNAS AND DONOR NUCLEIC ACIDS

In some embodiments, the first genome editing tool comprises a first genomic editor and at least one guide RNA (gRNA) that targets at least one genomic locus and that is cognate to the first genomic editor. In some embodiments, the first genome editing tool comprises a first genomic editor comprising a base editor, and at least one guide RNA (gRNA) that targets at least one genomic locus and that is cognate to the base editor.

In some embodiments, the second genome editing tool comprises a comprises a second genomic editor and at least one gRNA that targets at least one genomic locus and that is cognate to the second genomic editor, wherein the first genomic editor is orthogonal to the second genomic editor. In some embodiments, the second genome editing tool comprises a second genomic editor comprising an RNA-guided cleavase, and at least one gRNA that targets at least one genomic locus and that is cognate to the RNA-guided cleavase, wherein the base editor is orthogonal to the RNA-guided cleavase.

In some embodiments, the at least one gRNA that is cognate to the first genomic editor or the base editor is non-cognate to the second genomic editor or the RNA-guided cleavase. In some embodiments, the at least one gRNA that is cognate to the second genomic editor or the RNA-guided cleavase is non-cognate to the first genomic editor or the base editor.

In some embodiments, the at least one gRNA that is cognate to the first genomic editor or the base editor comprises at least two gRNAs that target at least two different genomic loci. In some embodiments, the at least one gRNA that is cognate to the second genomic editor or the RNA-guided cleavase comprises at least two gRNAs that target at least two different genomic loci. In some embodiments, the at least one gRNA that is cognate to the first genomic editor or the base editor comprises at least three gRNAs that target at least three different genomic loci. In some embodiments, the at least one gRNA that is cognate to the second genomic editor or the RNA-guided cleavase comprises at least three gRNAs that target at least three different genomic loci. In some embodiments, the at least one gRNA that is cognate to the first genomic editor or the base editor comprises at least four gRNAs that target at least four different genomic loci. In some embodiments, the at least one gRNA that is cognate to the second genomic editor or the RNA-guided cleavase comprises at least four gRNAs that target at least four different genomic loci. In some embodiments, the at least one gRNA that is cognate to the first genomic editor or the base editor comprises at least five gRNAs that target at least five different genomic loci. In some embodiments, the at least one gRNA that is cognate to the second genomic editor or the RNA-guided cleavase comprises at least five gRNAs that target at least five different genomic loci. In some embodiments, the at least one gRNA that is cognate to the first genomic editor or the base editor comprises at least six gRNAs that target at least six different genomic loci. In some embodiments, the first genomic editor and one, two, three, four, five, or six of the at least one gRNA that are cognate to the first genomic editor or the base editor and target different genomic loci are contained in a same lipid nanoparticle (LNP). In some embodiments, the base editor or the at least one gRNA that is cognate to the second genomic editor or the RNA-guided cleavase comprises at least six gRNAs that target at least six different genomic loci.

### A. Target Sequences and Genes

In some embodiments, the methods and compositions of the present disclosure utilize a CRISPR/Cas system to cleave a target sequence of at least one genomic loci targeted by a guide RNA. For example, a target sequence may be recognized and cleaved by a Cas nuclease. In some embodiments, a target sequence for a Cas nuclease is located near the nuclease's cognate PAM sequence. In some embodiments, a Class 2 Cas nuclease may be directed by a gRNA to a target sequence of a gene, where the gRNA hybridizes with and the Class 2 Cas protein cleaves the target sequence. In some embodiments, the guide RNA hybridizes with and a Class 2 Cas nuclease cleaves the target sequence adjacent to or comprising its cognate PAM. In some embodiments, the target sequence may be complementary to a targeting sequence of the guide RNA. In some embodiments, the degree of complementarity between a targeting sequence of a guide RNA and the portion of the corresponding target sequence that hybridizes to the guide RNA may be about 80%, 85%, 90%, 95%, 97%, 98%, 99%, or 100%. In some embodiments, the percent identity between a targeting sequence of a guide RNA and the portion of the corresponding target sequence that hybridizes to the guide RNA may be about 50%, 55%, 60%, 65%, 70%, 75%, 80%, 85%, 90%, 95%, 97%, 98%, 99%, or 100%. In some embodiments, the homology region of the target is adjacent to a cognate PAM sequence. In some embodiments, the target sequence may comprise a sequence 100% complementary with the targeting sequence of the guide RNA. In other embodiments, the target sequence may comprise at least one mismatch, deletion, or insertion, as compared to the targeting sequence of the guide RNA.

The length of the target sequence may depend on the nuclease system used. For example, the targeting sequence of a guide RNA for a CRISPR/Cas system may comprise 10, 11, 12, 13, 14, 15, 16, 17, 18, 19, 20, 21, 22, 23, 24, 25, 26, 27, 28, 29, 30, 35, 40, 45, 50, or more than 50 nucleotides in length and the target sequence is a corresponding length, optionally adjacent to a PAM sequence. In some embodiments, the target sequence may comprise 15-24 nucleotides in length. In some embodiments, the target sequence may comprise 17-21 nucleotides in length. In some embodiments, the target sequence may comprise 20 nucleotides in length. In some embodiments, the target sequence may comprise 24 nucleotides in length. When nickases are used, the target sequence may comprise a pair of target sequences recognized by a pair of nickases that cleave opposite strands of the DNA molecule. In some embodiments, the target sequence may comprise a pair of target sequences recognized by a pair of nickases that cleave the same strands of the DNA molecule. In some embodiments, the target sequence may comprise a part of target sequences recognized by one or more Cas nucleases.

The target nucleic acid molecule may be any DNA or RNA molecule that is endogenous or exogenous to a cell. In some embodiments, the target nucleic acid molecule may be an episomal DNA, a plasmid, a genomic DNA, viral genome, or chromosomal DNA. In some embodiments, the target sequence of the gene may be a genomic sequence from a cell or in a cell, including a human cell.

In further embodiments, the target sequence may be a viral sequence. In further embodiments, the target sequence may be a pathogen sequence. In yet other embodiments, the target sequence may be a synthesized sequence. In further embodiments, the target sequence may be a chromosomal sequence. In certain embodiments, the target sequence may comprise a translocation junction, e.g., a translocation associated with a cancer. In some embodiments, the target sequence may be on a eukaryotic chromosome, such as a human chromosome.

In some embodiments, the target sequence may be located in a genomic locus; for example, the target sequence may be located in a coding sequence of a gene, an intron sequence of a gene, a regulatory sequence, a transcriptional control sequence of a gene, a translational control sequence of a gene, a splicing site, or a non-coding sequence between genes (e.g., intergenic space). In some embodiments, the gene may be a protein coding gene. In other embodiments, the gene may be a non-coding RNA gene. In some embodiments, the target sequence may comprise all or a portion of a disease-associated gene. In some embodiments, the target sequence may be located in a non-genic functional site in the genome, for example a site that controls aspects of chromatin organization, such as a scaffold site or locus control region.

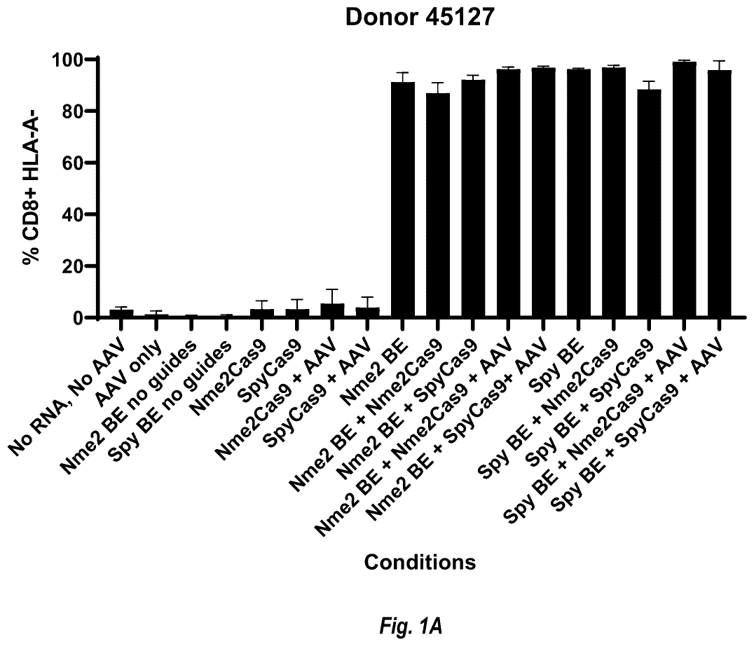

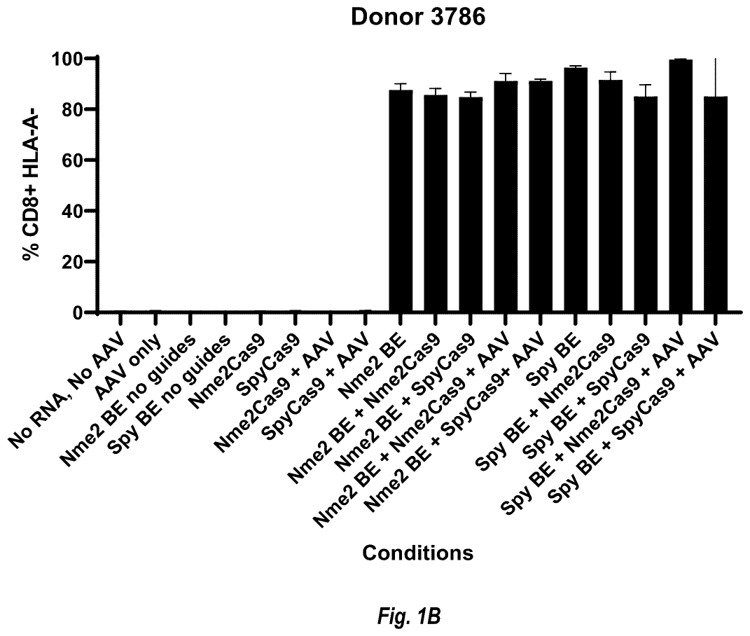

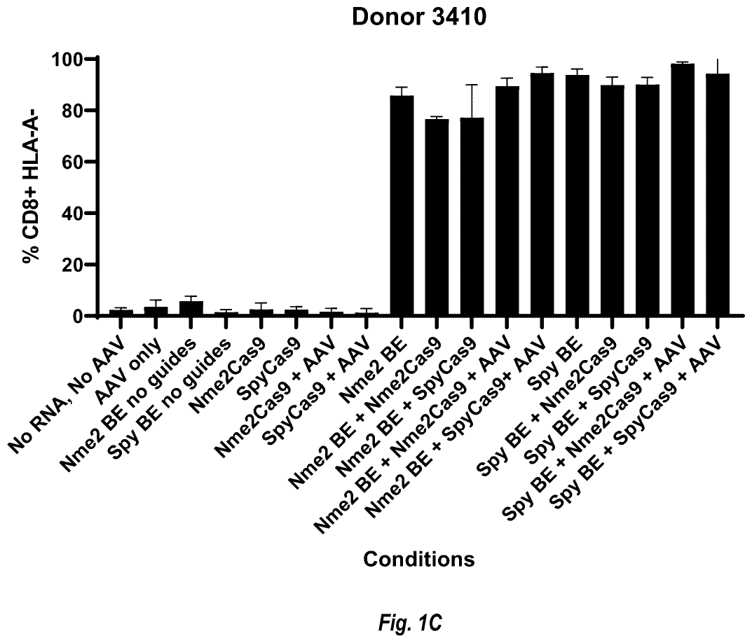

In some embodiments involving a Cas nuclease, such as a Class 2 Cas nuclease, the target sequence may be adjacent to a protospacer adjacent motif (“PAM”). In some embodiments, the PAM may be adjacent to or within 1, 2, 3, or 4, nucleotides of the 3′ end of the target sequence. The length and the sequence of the PAM may depend on the Cas protein used. For example, the PAM may be selected from a consensus or a particular PAM sequence for a specific Spy Cas9 protein or Spy Cas9 ortholog, including those disclosed in FIG. 1 of Ran et al., Nature, 520: 186-191 (2015), and Figure S5 of Zetsche 2015, the relevant disclosure of each of which is incorporated herein by reference. In some embodiments, the PAM may be 2, 3, 4, 5, 6, 7, 8, 9, or 10 nucleotides in length. Non-limiting exemplary PAM sequences include NGG, NGGNG, NG, NAAAAN, NNAAAAW, NNNNACA, GNNNCNNA, TTN, and NNNNGATT (wherein N is defined as any nucleotide, and W is defined as either A or T). In some embodiments, the PAM sequence may be NGG. In some embodiments, the PAM sequence may be NGGNG. In some embodiments, the PAM sequence may be TTN. In some embodiments, the PAM sequence may be NNAAAAW.

In some embodiments, the PAM may be selected from a consensus or a particular PAM sequence for a specific Nine Cas9 protein or Nine Cas9 ortholog (Edraki et al., 2019). In some embodiments, the Nine Cas9 PAM may comprise 2, 3, 4, 5, 6, 7, 8, 9, or 10 nucleotides in length. Non-limiting exemplary PAM sequences include NCC, N4GAYW, N4GYTT, N4GTCT, NNNNCC (a), NNNNCAAA (wherein N is defined as any nucleotide, W is defined as either A or T, and R is defined as either A or G; and (a) is a preferred, but not required, A after the second C)). In some embodiments, the PAM sequence may be NCC.

In some embodiments, the at least one gRNA that is cognate to the first genomic editor or the base editor or the at least one gRNA that is cognate to the second genomic editor or the RNA-guided cleavase comprises at least one single guide RNA (sgRNA). In some embodiments, the at least one gRNA that is cognate to the first genomic editor or the base editor or the at least one gRNA that is cognate to the second genomic editor or the RNA-guided cleavase is a short-single guide RNA (short-sgRNA) comprising a conserved portion of an sgRNA comprising a hairpin region, wherein the hairpin region lacks at least 5-10 nucleotides and wherein the short-sgRNA comprises a 5′ end modification or a 3′ end modification or both.

In some embodiments, the at least one gRNA that is cognate to the first genomic editor or the base editor targets one or more genes chosen from the TRBC locus, the HLA-A locus, the HLA-B locus, the CIITA locus, the HLA-DR locus, the HLA-DQ locus, and the HLA-DP locus. In some embodiments, the at least one gRNA that is cognate to the second genomic editor or the RNA-guided cleavase targets one or more genomic loci chosen from the TRAC locus, the AAVS1 locus, and the CIITA locus.

In some embodiments, (i) the at least one gRNA that is cognate to the first genomic editor or the base editor comprises a gRNA that targets the HLA-A locus and a gRNA that targets the CIITA locus, and the at least one gRNA that is cognate to the second genomic editor or the RNA-guided cleavase comprises a gRNA that targets the TRAC locus; (ii) the at least one gRNA that is cognate to the first genomic editor or the base editor comprises a gRNA that targets the TRBC locus, a gRNA that targets the HLA-A locus, and a gRNA that targets the CIITA locus, and the at least one gRNA that is cognate to the second genomic editor or the RNA-guided cleavase comprises a gRNA that targets the TRAC locus; (iii) the at least one gRNA that is cognate to the first genomic editor or the base editor comprises a gRNA that targets the HLA-A locus, a gRNA that targets the HLA-B locus, and a gRNA that targets the CIITA locus, and the at least one gRNA that is cognate to the second genomic editor or the RNA-guided cleavase comprises a gRNA that targets the TRAC locus; (iv) the at least one gRNA that is cognate to the first genomic editor or the base editor comprises a gRNA that targets the TRBC locus, a gRNA that targets the HLA-A locus, a gRNA that targets the HLA-B locus, and a gRNA that targets the CIITA locus, and the at least one gRNA that is cognate to the second genomic editor or the RNA-guided cleavase comprises a gRNA that targets the TRAC locus; (v) the at least one gRNA that is cognate to the first genomic editor or the base editor comprises a gRNA that targets the HLA-A locus and a gRNA that targets the HLA-DR locus, the HLA-DQ locus, or the HLA-DP locus, and the at least one gRNA that is cognate to the second genomic editor or the RNA-guided cleavase comprises a gRNA that targets the TRAC locus; (vi) the at least one gRNA that is cognate to the first genomic editor or the base editor comprises a gRNA that targets the TRBC locus, a gRNA that targets the HLA-A locus, and a gRNA that targets the HLA-DR locus, the HLA-DQ locus, or the HLA-DP locus, and the at least one gRNA that is cognate to the second genomic editor or the RNA-guided cleavase comprises a gRNA that targets the TRAC locus; (vii) the at least one gRNA that is cognate to the first genomic editor or the base editor comprises a gRNA that targets the HLA-A locus, a gRNA that targets the HLA-B locus, and a gRNA that targets the HLA-DR locus, the HLA-DQ locus, or the HLA-DP locus, and the at least one gRNA that is cognate to the second genomic editor or the RNA-guided cleavase comprises a gRNA that targets the TRAC locus; (viii) the at least one gRNA that is cognate to the first genomic editor or the base editor comprises a gRNA that targets the TRBC locus, a gRNA that targets the HLA-A locus, a gRNA that targets the HLA-B locus, and a gRNA that targets the HLA-DR locus, the HLA-DQ locus, or the HLA-DP locus, and the at least one gRNA that is cognate to the second genomic editor or the RNA-guided cleavase comprises a gRNA that targets the TRAC locus; (ix) the at least one gRNA that is cognate to the first genomic editor or the base editor comprises a gRNA that targets the TRAC locus, a gRNA that targets the TRBC locus, a gRNA that targets the CIITA locus, and a gRNA that targets the HLA-A locus, and the at least one gRNA that is cognate to the second genomic editor or the RNA-guided cleavase comprises a gRNA that targets the TRAC locus; (x) the at least one gRNA that is cognate to the first genomic editor or the base editor comprises a gRNA that targets the TRBC locus, a gRNA that targets the HLA-A locus, and a gRNA that targets the CIITA locus, and the at least one gRNA that is cognate to the second genomic editor or the RNA-guided cleavase comprises a gRNA that targets the AAVS1 locus; (xi) the at least one gRNA that is cognate to the first genomic editor or the base editor comprises a gRNA that targets the TRBC locus, a gRNA that targets the HLA-A locus, a gRNA that targets the HLA-B locus, and a gRNA that targets the CIITA locus, and the at least one gRNA that is cognate to the second genomic editor or the RNA-guided cleavase comprises a gRNA that targets the AAVS1 locus; (xii) the at least one gRNA that is cognate to the first genomic editor or the base editor comprises a gRNA that targets the TRBC locus, a gRNA that targets the HLA-A locus, and a gRNA that targets the HLA-DR locus, the HLA-DQ locus, or the HLA-DP locus, and the at least one gRNA that is cognate to the second genomic editor or the RNA-guided cleavase comprises a gRNA that targets the AAVS1 locus; (xiii) the at least one gRNA that is cognate to the first genomic editor or the base editor comprises a gRNA that targets the TRBC locus, a gRNA that targets the HLA-A locus, a gRNA that targets the HLA-B locus, and a gRNA that targets the HLA-DR locus, the HLA-DQ locus, or the HLA-DP locus, and the at least one gRNA that is cognate to the second genomic editor or the RNA-guided cleavase comprises a gRNA that targets the AAVS1 locus.

In some embodiments, in any one of subparts (i)-(ix) above, the at least one gRNA that is cognate to the second genomic editor or the RNA-guided cleavase comprises a further gRNA that targets the AAVS1 locus. In some embodiments, in any one of subparts (x)-(xiii) above, the at least one gRNA that is cognate to the second genomic editor or the RNA-guided cleavase comprises a further gRNA that targets the TRAC locus. In some embodiments, the cell is contacted with the further gRNA that targets the AAVS1 locus after the cell is contacted with the gRNA that targets the TRAC locus. In some embodiments, the cell is contacted with the further gRNA that targets the TRAC locus after the cell is contacted with the gRNA that targets the AAVS1 locus.

B. Modified gRNAs

In the case of a sgRNA, the above guide sequences may further comprise additional nucleotides to form a sgRNA, e.g., with the following exemplary nucleotide sequence following the 3′ end of the guide sequence: GUUUUAGAGCUAGAAAUAGCAAGUUAAAAUAAGGCUAGUCCGUUAUCAACUU GAAAAAGUGGCACCGAGUCGGUGC (SEQ ID NO: 226) in 5′ to 3′ orientation.

In the case of a sgRNA, the above guide sequences may further comprise additional nucleotides to form a sgRNA, e.g., with the following exemplary nucleotide sequence following the 3′ end of the guide sequence: GUUUUAGAGCUAGAAAUAGCAAGUUAAAAUAAGGCUAGUCCGUUAUCAACUU GAAAAAGUGGCACCGAGUCGGUGCUUUU (SEQ ID NO: 227) in 5′ to 3′ orientation.

In the case of a sgRNA, the guide sequences may be integrated into the following modified motif: mN*mN*m*NNNNNNNNNNNNNNGUUUUAGAmGmCmUmAmGmAmAmAmU mAmGmCAAGUUAAAAUAAGGCUAGUCCGUUAUCAmAmCmUmUmGmAmAmAm AmAmGmUmGmGmCmAmCmCmGmAmGmUmCmGmGmUmGmCmU*mU*mU*mU (SEQ ID NO: 228), where “N” may be any natural or non-natural nucleotide, preferably an RNA nucleotide; sugar moieties of the nucleotide can be ribose, deoxyribose, or similar compounds with substitutions; m is a 2′-O-methyl modified nucleotide, and * is a phosphorothioate linkage to the adjacent nucleotide residue; and wherein the N's are collectively the nucleotide sequence of a guide sequence. In the context of a modified sequence, unless otherwise indicated, A, C, G, N, and U are an unmodified RNA nucleotide, i.e., a 2′-OH sugar moiety with a phosphodiesterase linkage to the adjacent nucleotide residue, or a 5′-terminal P04.

In the case of a sgRNA, the guide sequences may further comprise a SpyCas9 sgRNA sequence. An example of a SpyCas9 sgRNA sequence is shown in Table YY (SEQ ID NO: 226: GUUUUAGAGC UAGAAAUAGC AAGUUAAAAU AAGGCUAGUC CGUUAUCAAC UUGAAAAAGU GGCACCGAGU CGGUGC—“Exemplary SpyCas9 sgRNA-1”), included at the 3′ end of the guide sequence, and provided with the domains as shown in Table YY below. LS is lower stem. B is bulge. US is upper stem. H1 and H2 are hairpin 1 and hairpin 2, respectively. Collectively H1 and H2 are referred to as the hairpin region. A model of the structure is provided in FIG. 10A of WO2019237069 which is incorporated herein by reference.

The nucleotide sequence of Exemplary SpyCas9 sgRNA-1 may serve as a template sequence for specific chemical modifications, sequence substitutions and truncations.

In certain embodiments, the gRNA is an sgRNA or a dgRNA, for example, and it optionally comprises a chemical modification. In some embodiments, the modified sgRNA comprises a guide sequence and a SpyCas9 sgRNA sequence, e.g., Exemplary SpyCas9 sgRNA-1. A gRNA, such as an sgRNA, may include modifications on the 5′ end of the guide sequence or on the 3′ end of the SpyCas9 sgRNA sequence, such as, e.g., Exemplary SpyCas9 sgRNA-1 at one or more of the terminal nucleotides, e.g., at 1, 2, 3, or 4 of the nucleotides at the 3′ end or at the 5′ end. In certain embodiments, the modified nucleotide is selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, or an inverted abasic modified nucleotide; or a combination thereof. In certain embodiments, the modified nucleotide includes a 2′-OMe modified nucleotide. In certain embodiments, the modified nucleotide includes a PS linkage. In certain embodiments, the modified nucleotide includes a 2′-OMe modified nucleotide and a PS linkage.

In certain embodiments, using SEQ ID NO: 226 (“Exemplary SpyCas9 sgRNA-1”) as an example, the Exemplary SpyCas9 sgRNA-1 further includes one or more of: (A) a shortened hairpin 1 region, or a substituted and optionally shortened hairpin 1 region, wherein (1) at least one of the following pairs of nucleotides are substituted in hairpin 1 with Watson-Crick pairing nucleotides: H1-1 and H1-12, H1-2 and H1-11, H1-3 and H1-10, or H1-4 and H1-9, and the hairpin 1 region optionally lacks (a) any one or two of H1-5 through H1-8, (b) one, two, or three of the following pairs of nucleotides: H1-1 and H1-12, H1-2 and H1-11, H1-3 and H1-10, and H1-4 and H1-9, or (c) 1-8 nucleotides of hairpin 1 region; or (2) the shortened hairpin 1 region lacks 4-8 nucleotides, preferably 4-6 nucleotides, and (a) one or more of positions H1-1, H1-2, or H1-3 is deleted or substituted relative to Exemplary SpyCas9 sgRNA-1 (SEQ ID NO: 226), or (b) one or more of positions H1-6 through H1-10 is substituted relative to Exemplary SpyCas9 sgRNA-1 (SEQ ID NO: 226); or (3) the shortened hairpin 1 region lacks 5-10 nucleotides, preferably 5-6 nucleotides, and one or more of positions N18, H1-12, or n is substituted relative to Exemplary SpyCas9 sgRNA-1 (SEQ ID NO: 226); or (B) a shortened upper stem region, wherein the shortened upper stem region lacks 1-6 nucleotides and wherein the 6, 7, 8, 9, 10, or 11 nucleotides of the shortened upper stem region include less than or equal to 4 substitutions relative to Exemplary SpyCas9 sgRNA-1 (SEQ ID NO: 226); or (C) a substitution relative to Exemplary SpyCas9 sgRNA-1 (SEQ ID NO: 226) at any one or more of LS6, LS7, US3, US10, B3, N7, N15, N17, H2-2 and H2-14, wherein the substituent nucleotide is neither a pyrimidine that is followed by an adenine, nor an adenine that is preceded by a pyrimidine; or (D) an Exemplary SpyCas9 sgRNA-1 (SEQ ID NO: 226) with an upper stem region, wherein the upper stem modification comprises a modification to any one or more of US1-US12 in the upper stem region, wherein (1) the modified nucleotide is optionally selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or a combination thereof, or (2) the modified nucleotide optionally includes a 2′-OMe modified nucleotide.

In some embodiments, the sgRNA comprises a modified motif disclosed herein, including the modified motif of any one of SEQ ID NOs: 228-242 and 246-250, 312-314 or any other modified motif shown in the Table of Sequences, where “N” may be any natural or non-natural nucleotide, preferably an RNA nucleotide; sugar moieties of the nucleotide can be ribose, deoxyribose, or similar compounds with substitutions; m is a 2′-O-methyl modified nucleotide, and * is a phosphorothioate linkage to the adjacent nucleotide residue; and wherein the N's are collectively the nucleotide sequence of a guide sequence.

In certain embodiments, using SEQ ID NO: 400 (“Exemplary NmeCas9 sgRNA-1” as shown in Table 20) as an example, the Exemplary NmeCas9 sgRNA-1 includes: (A) A guide RNA (gRNA) comprising a guide region and a conserved region, the conserved region comprising one or more of: (a) a shortened repeat/anti-repeat region, wherein the shortened repeat/anti-repeat region lacks 2-24 nucleotides, wherein (i) one or more of nucleotides 37-48 and 53-64 is deleted and optionally one or more of nucleotides 37-64 is substituted relative to SEQ ID NO: 400; and (ii) nucleotide 36 is linked to nucleotide 65 by at least 2 nucleotides; or (b) a shortened hairpin 1 region, wherein the shortened hairpin 1 lacks 2-10, optionally 2-8 nucleotides, wherein (i) one or more of nucleotides 82-86 and 91-95 is deleted and optionally one or more of positions 82-96 is substituted relative to SEQ ID NO: 400; and (ii) nucleotide 81 is linked to nucleotide 96 by at least 4 nucleotides; or (c) a shortened hairpin 2 region, wherein the shortened hairpin 2 lacks 2-18, optionally 2-16 nucleotides, wherein (i) one or more of nucleotides 113-121 and 126-134 is deleted and optionally one or more of nucleotides 113-134 is substituted relative to SEQ ID NO: 400; and (ii) nucleotide 112 is linked to nucleotide 135 by at least 4 nucleotides; wherein one or both nucleotides 144-145 are optionally deleted relative to SEQ ID NO: 400; wherein optionally at least 10 nucleotides are modified nucleotides.

Exemplary unmodified conserved portion nucleotide sequences include:

(SEQ ID NO: 243)
GUUGUAGCUCCCUUUCUCAUUUCGGAAACGAAAUGAGAACCGUUGCUAC
AAUAAGGCCGUCUGAAAAGAUGUGCCGCAACGCUCUGCCCCUUAAAGCU
UCUGCUUUAAGGGGCAUCGUUUA;
(SEQ ID NO: 244)
GUUGUAGCUCCCUGAAACCGUUGCUACAAUAAGGCCGUCGAAAGAUGUG
CCGCAACGCUCUGCCUUCUGGCAUCGUU,
and
(SEQ ID NO: 245)
GUUGUAGCUCCCUGGAAACCCGUUGCUACAAUAAGGCCGUCGAAAGAUG
UGCCGCAACGCUCUGCCUUCUGGCAUCGUUUAUU.

In the case of a sgRNA, the guide sequences may be integrated into one of the following exemplary modified conserved portion motifs:

(SEQ ID NO: 246)
GUUGmUmAmGmCUCCCmUmGmAmAmAmCmCGUUmGmCUAmCAAU*AAGm
GmCCmGmUmCmGmAmAmAmGmAmUGUGCmCGCmAmAmCmGCUCUmGmCC
mUmUmCmUGmGCmAmUC*mG*mU*mU
and
(SEQ ID NO: 247)
GUUGmUmAmGmCUCCCmUmGmAmAmAmCmCGUUmGmCUAmCAAU*AAGm
GmCCmGmUmCmGmAmAmAmGmAmUGUGCmCGmCAAmCGCUCUmGmCCmU
mUmCmUGGCAUCG*mU*mU.

In certain embodiments, the guide sequence is 20-25 nucleotides in length ((N)20-25), wherein each nucleotide may be independently modified. In certain embodiments, each of nucleotides 1-3 of the 5′ end of the guide is independently modified. In certain embodiments, each of nucleotides 1-3 of the 5′ end of the guide is independently modified with a 2′-OMe modification. In certain embodiments, each of nucleotides 1-3 of the 5′ end of the guide is independently modified with a phosphorothioate linkage to the adjacent nucleotide residue. In certain embodiments, each of nucleotides 1-3 of the 5′ end of the guide is independently modified with a 2′-OMe modification and a phosphorothioate linkage to the adjacent nucleotide residue.

In the case of a sgRNA, modified guide sequences may be integrated into one of the following exemplary modified conserved portion motifs:

(SEQ ID NO: 248)
mN*mNNNNNNNNmNNNmNNNNNNNNNNNNmGUUGmUmAmGmCUCCCmUm
GmAmAmAmCmCGUUmGmCUAmCAAU*AAGmGmCCmGmUmCmGmAmAmAm
GmAmUGUGCmCGCmAmAmCmGCUCUmGmCCmUmUmCmUGmGCmAmUC*m
G*mU*mU;
(SEQ ID NO: 249)
(N)₂₀₋₂₅ GUUGmUmAmGmCUCCCmUmGmAmAmAmCmCGUUmGmCUAmCA
AU*AAGmGmCCmGmUmCmGmAmAmAmGmAmUGUGCmCGCmAmAmCmGCU
CUmGmCCmUmUmCmUGmGCmAmUC*mG*mU*mU;
(SEQ ID NO: 250)
mN*mN*mN*mNmNNNmNmNNmNNmNNNNNmNNNNmNNNmGUUGmUmAmG
mCUCCCmUmGmAmAmAmCmCGUUmGmCUAmCAAU*AAGmGmCCmGmUmC
mGmAmAmAmGmAmUGUGCmCGmCAAmCGCUCUmGmCCmUmUmCmUGGCA
UCG*mU*mU;
or
any one of
(SEQ ID NO: 312)
mN*mN*mN*mNmNNNmNmNNmNNmNNNNNmNNNNmNNNmGUUGmUmAmG
mCUCCCmUmGmAmAmAmCmCGUUmGmCUAmCAAUAAGmGmCCmGmUmCm
GmAmAmAmGmAmUGUGCmCGmCAAmCGCUCUmGmCCmUmUmCmUGGCAU
CG*mU*mU,
(SEQ ID NO: 313)
mN*mN*mN*mNmNNNmNmNNmNNmNNNNNmNNNNmNNNmGUUGmUmAmG
mCUCCCmUmGmAmAmAmCmCGUUmGmCUAmCAAU*AAGmGmCCmGmUmC
mGmAmAmAmGmAmUGUGCmCGmCAAmCGmCmUmCmUmGmCCmUmUmCmU
GGCAUCG*mU*mU;
(SEQ ID NO: 314)
mN*mN*mN*mNmNNNmNmNNmNNmNNNNNmNNNNmNNNmGUUGmUmAmG
mCUCCCmUmGmAmAmAmCmCGUUmGmCUAmCAAUAAGmGmCCmGmUmCm
GmAmAmAmGmAmUGUGCmCGmCAAmCGmCmUmCmUmGmCCmUmUmCmUG
GCAUCG*mU*mU.

In certain embodiments, Exemplary SpyCas9 sgRNA-1, or an sgRNA, such as an sgRNA comprising an Exemplary SpyCas9 sgRNA-1, further includes a 3′ tail, e.g., a 3′ tail of 1, 2, 3, 4, or more nucleotides. In certain embodiments, the tail includes one or more modified nucleotides. In certain embodiments, the modified nucleotide is selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide; or a combination thereof. In certain embodiments, the modified nucleotide includes a 2′-OMe modified nucleotide. In certain embodiments, the modified nucleotide includes a PS linkage between nucleotides. In certain embodiments, the modified nucleotide includes a 2′-OMe modified nucleotide and a PS linkage between nucleotides.

In certain embodiments, the hairpin region includes one or more modified nucleotides. In certain embodiments, the modified nucleotide is selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide; or a combination thereof. In certain embodiments, the modified nucleotide includes a 2′-OMe modified nucleotide.

In certain embodiments, the upper stem region includes one or more modified nucleotides. In certain embodiments, the modified nucleotide selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide; or a combination thereof. In certain embodiments, the modified nucleotide includes a 2′-OMe modified nucleotide.

In certain embodiments, the Exemplary SpyCas9 sgRNA-1 comprises one or more YA dinucleotides, wherein Y is a pyrimidine, wherein the YA dinucleotide includes a modified nucleotide. In certain embodiments, the modified nucleotide selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or a combination thereof. In certain embodiments, the modified nucleotide includes a 2′-OMe modified nucleotide.

In certain embodiments, the Exemplary SpyCas9 sgRNA-1 comprises one or more YA dinucleotides, wherein Y is a pyrimidine, wherein the YA dinucleotide includes a sequence substituted nucleotide, wherein the pyrimidine is substituted for a purine. In certain embodiments, when the pyrimidine forms a Watson-Crick base pair in the single guide, the Watson-Crick based nucleotide of the sequence substituted pyrimidine nucleotide is substituted to maintain Watson-Crick base pairing.

In some embodiments, the gRNA is chemically modified. A gRNA comprising one or more modified nucleosides or nucleotides is called a “modified” gRNA or “chemically modified” gRNA, to describe the presence of one or more non-naturally or naturally occurring components or configurations that are used instead of or in addition to the canonical A, G, C, and U residues. In some embodiments, a modified gRNA is synthesized with a non-canonical nucleoside or nucleotide, is here called “modified.” Modified nucleosides and nucleotides can include one or more of: (i) alteration, e.g., replacement, of one or both of the non-linking phosphate oxygens or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) modification or replacement of a naturally occurring nucleobase, including with a non-canonical nucleobase (an exemplary base modification); and (iv) modification of the 3′ end or 5′ end of the oligonucleotide to provide exonuclease stability, e.g., with 2′ O-me, 2′ halide, or 2′ deoxy substituted ribose; or inverted abasic terminal nucleotide, or replacement of phosphodiester with phosphothioate.

Chemical modifications such as those listed above can be combined to provide modified gRNAs or mRNAs comprising nucleosides and nucleotides (collectively “residues”) that can have two, three, four, or more modifications. For example, a modified residue can have a modified sugar and a modified nucleobase. In certain embodiments, all, or substantially all, of the phosphate groups of a gRNA molecule are replaced with phosphorothioate groups. In some embodiments, modified gRNAs comprise at least one modified residue at or near the 5′ end of the RNA. In some embodiments, modified gRNAs comprise at least one modified residue at or near the 3′ end of the RNA.

In some embodiments, the gRNA comprises one, two, three or more modified residues. In some embodiments, at least 5% (e.g., at least 5%, 10%, 15%, preferably at least 20%, 25%, 30%, 35%, 40%, 45%, or 50%) of the positions in a modified gRNA are modified nucleosides or nucleotides. In some embodiments, at least 5% of the positions in the modified guide RNA are modified nucleotides or nucleosides. In some embodiments, at least 10% of the positions in the modified guide RNA are modified nucleotides or nucleosides. In some embodiments at least 15% of the positions in the modified gRNA are modified nucleotides or nucleosides. In some embodiments preferably at least 20% of the positions in the modified gRNA are modified nucleotides or nucleosides. In some embodiments, no more than 65% of the positions in the modified gRNA are modified nucleotides. In some embodiments, no more than 55% of the positions in the modified gRNA are modified nucleotides. In some embodiments, no more than 50% of the positions in the modified gRNA are modified nucleotides. In some embodiments, 10-70% of the positions in the modified gRNA are modified nucleotides. In some embodiments, 20-70% of the positions in the modified gRNA are modified nucleotides. In some embodiments, 20-50% of the positions in the modified gRNA are modified nucleotides and the nuclease is a SpyCas9 nuclease. In some embodiments, 30-70% of the positions in the modified gRNA are modified nucleotides and the nuclease is an NmeCas9 nuclease.

Unmodified nucleic acids can be prone to degradation by, e.g., intracellular nucleases or those found in serum. For example, nucleases can hydrolyze nucleic acid phosphodiester bonds. Accordingly, in one aspect the gRNAs described herein can contain one or more modified nucleosides or nucleotides, e.g., to introduce stability toward intracellular or serum-based nucleases. In some embodiments, the modified gRNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly the interferons, and cell death.

In some embodiments of a backbone modification, the phosphate group of a modified residue can be modified by replacing one or more of the oxygens with a different substituent. Further, the modified residue, e.g., modified residue present in a modified nucleic acid, can include the replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include, phosphorothioate, borano phosphate esters, methyl phosphonates, phosphoroamidates, phosphodithioate, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications, e.g., an amide linkage. In some embodiments, the charged phosphate group can be replaced by a neutral moiety. Examples of moieties which can replace the phosphate group can include, without limitation, e.g., methyl phosphonate, carboxymethyl, carbamate, amide, thioether. Further examples of moieties which can replace the phosphate group can include, without limitation, e.g., ethylene oxide linker, sulfonate, sulfonamide, thioformacetal, formacetal, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group, i.e. at sugar modification. For example, the 2′ hydroxyl group (OH) can be modified, e.g. replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH2CH2O)nCH2CH2OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the 2′ hydroxyl group modification can be 2′-O-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a C1-6 alkylene or C1-6 heteroalkylene bridge, to the 4′ carbon of the same ribose sugar, where exemplary bridges can include methylene, propylene, ether, or amino bridges; O-amino (wherein amino can be, e.g., NH2; alkylamino, dialkylamino, heterocyclyl, arylamino, diarylamino, heteroarylamino, or diheteroarylamino, ethylenediamine, or polyamino) and aminoalkoxy, O(CH2)n-amino, (wherein amino can be, e.g., NH2; alkylamino, dialkylamino, heterocyclyl, arylamino, diarylamino, heteroarylamino, or diheteroarylamino, ethylenediamine, or polyamino). In some embodiments, the 2′ hydroxyl group modification can include “unlocked” nucleic acids (UNA) in which the ribose ring lacks the C2′-C3′ bond. In some embodiments, the 2′ hydroxyl group modification can include the methoxyethyl group (MOE), (OCH2CH2OCH3, e.g., a PEG derivative). 2′ modifications can include hydrogen (i.e. deoxyribose sugars); halo (e.g., bromo, chloro, fluoro, or iodo); amino (wherein amino can be, e.g., NH2; alkylamino, dialkylamino, heterocyclyl, arylamino, diarylamino, heteroarylamino, diheteroarylamino, or amino acid); NH(CH2CH2NH)nCH2CH2-amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g. L-nucleosides. As used herein, a single abasic sugar is not understood to result in a discontinuity of a duplex.

In certain embodiments, 2′ modifications, include, for example, modifications include 2′-OMe, 2′-F, 2′-H, optionally 2′-O-Me.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA or tracr RNA. In embodiments comprising an sgRNA, one or more residues at one or both ends of the sgRNA may be chemically modified, or internal nucleosides may be modified, or the sgRNA may be chemically modified throughout. Certain embodiments comprise a 5′ end modification. Certain embodiments comprise a 3′ end modification. Certain embodiments comprise a 5′ end modification and a 3′ end modification.

In some embodiments, the guide RNAs disclosed herein comprise one of the modification patterns disclosed in WO2018/107028, the contents of which are hereby incorporated by reference in their entirety. In some embodiments, the guide RNAs disclosed herein comprise one of the structures/modification patterns disclosed in US20170114334, the contents of which are hereby incorporated by reference in their entirety. In some embodiments, the guide RNAs disclosed herein comprise one of the structures/modification patterns disclosed in WO2017/136794, the contents of which are hereby incorporated by reference in their entirety. In some embodiments, the guide RNAs disclosed herein comprise one of the structures/modification patterns disclosed in WO2019/237069, the contents of which are hereby incorporated by reference in their entirety. In some embodiments, the guide RNAs disclosed herein comprise one of the structures/modification patterns disclosed in WO2021/119275, the contents of which are hereby incorporated by reference in their entirety. In some embodiments, the guide RNAs disclosed herein comprise one of the structures/modification patterns disclosed in U.S. Application No. 63/275,426, the contents of which are hereby incorporated by reference in their entirety.

### C. Exemplary Guide RNAs, Compositions, Methods, and Engineered Cells for AAVS1 Editing

The disclosure provides a guide RNA that target the AAVS1 locus. Guide sequences targeting the AAVS1 locus are shown in Table 5 at SEQ ID NOs: 251-264.

In some embodiments, the guide sequences are complementary to the corresponding genomic region shown in the Table 5 below, according to coordinates from human reference genome hg38. Guide sequences of further embodiments may be complementary to sequences in the close vicinity of the genomic coordinate listed in Table 5. For example, guide sequences of further embodiments may be complementary to sequences that comprise 15 consecutive nucleotides±10 nucleotides of a genomic coordinate listed in Table 5.

In some embodiments, the guide sequences may further comprise additional nucleotides to form a sgRNA, e.g., with the following exemplary nucleotide sequence following the 3′ end of the guide sequence: GUUUUAGAGCUAGAAAUAGCAAGUUAAAAUAAGGCUAGUCCGUUAUCAACUU GAAAAAGUGGCACCGAGUCGGUGCUUUU (SEQ ID NO: 227) in 5′ to 3′ orientation. The guide sequences may further comprise additional nucleotides to form a sgRNA.

In some embodiments, the sgRNA comprises the modification pattern shown below in SEQ ID NO: 141, where N is any natural or non-natural nucleotide, and where the totality of the N's comprise a guide sequence as described herein and the modified sgRNA comprises the following sequence: mN*mN*m*NNNNNNNNNNNNNNGUUUUAGAmGmCmUmAmGmAmAmAmU mAmGmCAAGUUAAAAUAAGGCUAGUCCGUUAUCAmAmCmUmUmGmAmAmAm AmAmGmUmGmGmCmAmCmCmGmAmGmUmCmGmGmUmGmCmU*mU*mU*mU (SEQ ID NO: 228), where “N” may be any natural or non-natural nucleotide. For example, encompassed herein is SEQ ID NO: 228, where the N's are replaced with any of the guide sequences disclosed herein. The modifications remain as shown in SEQ ID NO: 141 despite the substitution of N's for the nucleotides of a guide. That is, although the nucleotides of the guide replace the “N's”, the first three nucleotides are 2′OMe modified and there are phosphorothioate linkages between the first and second nucleotides, the second and third nucleotides and the third and fourth nucleotides.

In some embodiments, the gRNA targeting TRAC comprises a guide sequence chosen from: i) SEQ ID NOs: 251-264; ii) at least 17, 18, 19, or 20 contiguous nucleotides of a sequence selected from SEQ ID NOs: 251-264; iii) a guide sequence at least 95%, 90%, or 85% identical to a sequence selected from SEQ ID NOs: 251-264; iv) a sequence that comprises 10 contiguous nucleotides±10 nucleotides of a genomic coordinate listed in Table 5; v) at least 17, 18, 19, or 20 contiguous nucleotides of a sequence from (iv); or vi) a guide sequence that is at least 95%, 90%, or 85% identical to a sequence selected from (v).

In some embodiments, the guide sequence comprises SEQ ID NO: 251. In some embodiments, the guide sequence comprises SEQ ID NO: 252. In some embodiments, the guide sequence comprises SEQ ID NO: 253. In some embodiments, the guide sequence comprises SEQ ID NO: 254. In some embodiments, the guide sequence comprises SEQ ID NO: 255. In some embodiments, the guide sequence comprises SEQ ID NO: 256. In some embodiments, the guide sequence comprises SEQ ID NO: 257. In some embodiments, the guide sequence comprises SEQ ID NO: 258. In some embodiments, the guide sequence comprises SEQ ID NO: 259. In some embodiments, the guide sequence comprises SEQ ID NO: 260. In some embodiments, the guide sequence comprises SEQ ID NO: 261. In some embodiments, the guide sequence comprises SEQ ID NO: 262. In some embodiments, the guide sequence comprises SEQ ID NO: 263. In some embodiments, the guide sequence comprises SEQ ID NO: 264. In some embodiments, the guide sequence comprises SEQ ID NO: 265. In some embodiments, the guide sequence comprises SEQ ID NO: 266. In some embodiments, the guide sequence comprises SEQ ID NO: 267. In some embodiments, the guide sequence comprises SEQ ID NO: 268. In some embodiments, the guide sequence comprises SEQ ID NO: 269. In some embodiments, the guide sequence comprises SEQ ID NO: 270. In some embodiments, the guide sequence comprises SEQ ID NO: 271. In some embodiments, the guide sequence comprises SEQ ID NO: 272. In some embodiments, the guide sequence comprises SEQ ID NO: 273. In some embodiments, the guide sequence comprises SEQ ID NO: 274. In some embodiments, the guide sequence comprises SEQ ID NO: 275. In some embodiments, the guide sequence comprises SEQ ID NO: 276. In some embodiments, the guide sequence comprises SEQ ID NO: 277. In some embodiments, the guide sequence comprises SEQ ID NO: 278. In some embodiments, the guide sequence comprises SEQ ID NO: 279. In some embodiments, the guide sequence comprises SEQ ID NO: 280. In some embodiments, the guide sequence comprises SEQ ID NO: 281. In some embodiments, the guide sequence comprises SEQ ID NO: 282. In some embodiments, the guide sequence comprises SEQ ID NO: 283. In some embodiments, the guide sequence comprises SEQ ID NO: 284. In some embodiments, the guide sequence comprises SEQ ID NO: 285. In some embodiments, the guide sequence comprises SEQ ID NO: 286. In some embodiments, the guide sequence comprises SEQ ID NO: 287. In some embodiments, the guide sequence comprises SEQ ID NO: 288. In some embodiments, the guide sequence comprises SEQ ID NO: 289. In some embodiments, the guide sequence comprises SEQ ID NO: 290. In some embodiments, the guide sequence comprises SEQ ID NO: 291. In some embodiments, the guide sequence comprises SEQ ID NO: 292.

TABLE 5
AAVS1 guide sequences, guide RNA sequences, and
chromosomal coordinates

| Guide | SEQ ID | | Exemplary | | |
|---|---|---|---|---|---|
| ID | NO to the | | Full Sequence | | Genomic |
| | Guide | Guide | (SEQ ID NOS: | Exemplary Mod Sequence | Coordinates |
| | Sequence | Sequence | 265-278) | (SEQ ID NOS: 279-292) | (hg38) |
| G000562 | 251 | CCAAUA | CCAAUAUCAGG | mC*mC*mA*AUAUCAGGA | chr19: |
| | | UCAGGA | AGACUAGGAGU | GACUAGGAGUUUUAGAm | 55115695- |
| | | GACUAG | UUUAGAGCUAG | GmCmUmAmGmAmAmAm | 55115715 |
| | | GA | AAAUAGCAAGU | UmAmGmCAAGUUAAAAU | |
| | | | UAAAAUAAGGC | AAGGCUAGUCCGUUAUC | |
| | | | UAGUCCGUUAU | AmAmCmUmUmGmAmAm | |
| | | | CAACUUGAAAA | AmAmAmGmUmGmGmCm | |
| | | | AGUGGCACCGA | AmCmCmGmAmGmUmCm | |
| | | | GUCGGUGCUUU | GmGmUmGmCmU*mU*mU | |
| | | | U | *mU | |
| G013515 | 252 | CCAUCG | CCAUCGUAAGC | mC*mC*mA*UCGUAAGCA | chr19: |
| | | UAAGCA | AAACCUUAGGU | AACCUUAGGUUUUAGAm | 55115588- |
| | | AACCUU | UUUAGAGCUAG | GmCmUmAmGmAmAmAm | 55115608 |
| | | AG | AAAUAGCAAGU | UmAmGmCAAGUUAAAAU | |
| | | | UAAAAUAAGGC | AAGGCUAGUCCGUUAUC | |
| | | | UAGUCCGUUAU | AmAmCmUmUmGmAmAm | |
| | | | CAACUUGAAAA | AmAmAmGmUmGmGmCm | |
| | | | AGUGGCACCGA | AmCmCmGmAmGmUmCm | |
| | | | GUCGGUGCUUU | GmGmUmGmCmU*mU*mU | |
| | | | U | *mU | |
| G013519 | 253 | GCAAGG | GCAAGGAGAGA | mG*mC*mA*AGGAGAGA | chr19: |
| | | AGAGA | GAUGGCUCCGU | GAUGGCUCCGUUUUAGA | 55115616- |
| | | GAUGGC | UUUAGAGCUAG | mGmCmUmAmGmAmAmA | 55115636 |
| | | UCC | AAAUAGCAAGU | mUmAmGmCAAGUUAAA | |
| | | | UAAAAUAAGGC | AUAAGGCUAGUCCGUUA | |
| | | | UAGUCCGUUAU | UCAmAmCmUmUmGmAm | |
| | | | CAACUUGAAAA | AmAmAmAmGmUmGmGm | |
| | | | AGUGGCACCGA | CmAmCmCmGmAmGmUm | |
| | | | GUCGGUGCUUU | CmGmGmUmGmCmU*mU* | |
| | | | U | mU*mU | |
| G013520 | 254 | GAGAG | GAGAGAUGGCU | mG*mA*mG*AGAUGGCUC | chr19: |
| | | AUGGCU | CCAGGAAAUGU | CAGGAAAUGUUUUAGAm | 55115623- |
| | | CCAGGA | UUUAGAGCUAG | GmCmUmAmGmAmAmAm | 55115643 |
| | | AAU | AAAUAGCAAGU | UmAmGmCAAGUUAAAAU | |
| | | | UAAAAUAAGGC | AAGGCUAGUCCGUUAUC | |
| | | | UAGUCCGUUAU | AmAmCmUmUmGmAmAm | |
| | | | CAACUUGAAAA | AmAmAmGmUmGmGmCm | |
| | | | AGUGGCACCGA | AmCmCmGmAmGmUmCm | |
| | | | GUCGGUGCUUU | GmGmUmGmCmU*mU*mU | |
| | | | U | *mU | |
| G013523 | 255 | GGUGAC | GGUGACACACC | mG*mG*mU*GACACACCC | chr19: |
| | | ACACCC | CCCAUUUCCGU | CCAUUUCCGUUUUAGAm | 55115637- |
| | | CCAUUU | UUUAGAGCUAG | GmCmUmAmGmAmAmAm | 55115657 |
| | | CC | AAAUAGCAAGU | UmAmGmCAAGUUAAAAU | |
| | | | UAAAAUAAGGC | AAGGCUAGUCCGUUAUC | |
| | | | UAGUCCGUUAU | AmAmCmUmUmGmAmAm | |
| | | | CAACUUGAAAA | AmAmAmGmUmGmGmCm | |
| | | | AGUGGCACCGA | AmCmCmGmAmGmUmCm | |
| | | | GUCGGUGCUUU | GmGmUmGmCmU*mU*mU | |
| | | | U | *mU | |
| G013533 | 256 | AGACCC | AGACCCAAUAU | mA*mG*mA*CCCAAUAUC | chr19: |
| | | AAUAUC | CAGGAGACUGU | AGGAGACUGUUUUAGAm | 55115691- |
| | | AGGAG | UUUAGAGCUAG | GmCmUmAmGmAmAmAm | 55115711 |
| | | ACU | AAAUAGCAAGU | UmAmGmCAAGUUAAAAU | |
| | | | UAAAAUAAGGC | AAGGCUAGUCCGUUAUC | |
| | | | UAGUCCGUUAU | AmAmCmUmUmGmAmAm | |
| | | | CAACUUGAAAA | AmAmAmGmUmGmGmCm | |
| | | | AGUGGCACCGA | AmCmCmGmAmGmUmCm | |
| | | | GUCGGUGCUUU | GmGmUmGmCmU*mU*mU | |
| | | | U | *mU | |
| G013543 | 257 | UGUCCC | UGUCCCUAGUG | mU*mG*mU*CCCUAGUGG | chr19: |
| | | UAGUG | GCCCCACUGGU | CCCCACUGGUUUUAGAm | 55115755- |
| | | GCCCCA | UUUAGAGCUAG | GmCmUmAmGmAmAmAm | 55115775 |
| | | CUG | AAAUAGCAAGU | UmAmGmCAAGUUAAAAU | |
| | | | UAAAAUAAGGC | AAGGCUAGUCCGUUAUC | |
| | | | UAGUCCGUUAU | AmAmCmUmUmGmAmAm | |
| | | | CAACUUGAAAA | AmAmAmGmUmGmGmCm | |
| | | | AGUGGCACCGA | AmCmCmGmAmGmUmCm | |
| | | | GUCGGUGCUUU | GmGmUmGmCmU*mU*mU | |
| | | | U | *mU | |
| G013559 | 258 | CCGGCC | CCGGCCCUGGG | mC*mC*mG*GCCCUGGGA | chr19: |
| | | CUGGGA | AAUAUAAGGGU | AUAUAAGGGUUUUAGAm | 55115823- |
| | | AUAUA | UUUAGAGCUAG | GmCmUmAmGmAmAmAm | 55115843 |
| | | AGG | AAAUAGCAAGU | UmAmGmCAAGUUAAAAU | |
| | | | UAAAAUAAGGC | AAGGCUAGUCCGUUAUC | |
| | | | UAGUCCGUUAU | AmAmCmUmUmGmAmAm | |
| | | | CAACUUGAAAA | AmAmAmGmUmGmGmCm | |
| | | | AGUGGCACCGA | AmCmCmGmAmGmUmCm | |
| | | | GUCGGUGCUUU | GmGmUmGmCmU*mU*mU | |
| | | | U | *mU | |
| G013562 | 259 | AAUAU | AAUAUAAGGUG | mA*mA*mU*AUAAGGUG | chr19: |
| | | AAGGU | GUCCCAGCUGU | GUCCCAGCUGUUUUAGA | 55115834- |
| | | GGUCCC | UUUAGAGCUAG | mGmCmUmAmGmAmAmA | 55115854 |
| | | AGCU | AAAUAGCAAGU | mUmAmGmCAAGUUAAA | |
| | | | UAAAAUAAGGC | AUAAGGCUAGUCCGUUA | |
| | | | UAGUCCGUUAU | UCAmAmCmUmUmGmAm | |
| | | | CAACUUGAAAA | AmAmAmAmGmUmGmGm | |
| | | | AGUGGCACCGA | CmAmCmCmGmAmGmUm | |
| | | | GUCGGUGCUUU | CmGmGmUmGmCmU*mU* | |
| | | | U | mU*mU | |
| G013563 | 260 | AUAUA | AUAUAAGGUGG | mA*mU*mA*UAAGGUGG | chr19: |
| | | AGGUG | UCCCAGCUCGU | UCCCAGCUCGUUUUAGA | 55115835- |
| | | GUCCCA | UUUAGAGCUAG | mGmCmUmAmGmAmAmA | 55115855 |
| | | GCUC | AAAUAGCAAGU | mUmAmGmCAAGUUAAA | |
| | | | UAAAAUAAGGC | AUAAGGCUAGUCCGUUA | |
| | | | UAGUCCGUUAU | UCAmAmCmUmUmGmAm | |
| | | | CAACUUGAAAA | AmAmAmAmGmUmGmGm | |
| | | | AGUGGCACCGA | CmAmCmCmGmAmGmUm | |
| | | | GUCGGUGCUUU | CmGmGmUmGmCmU*mU* | |
| | | | U | mU*mU | |
| G013564 | 261 | UAUAA | UAUAAGGUGGU | mU*mA*mU*AAGGUGGU | chr19: |
| | | GGUGG | CCCAGCUCGGU | CCCAGCUCGGUUUUAGA | 55115836- |
| | | UCCCAG | UUUAGAGCUAG | mGmCmUmAmGmAmAmA | 55115856 |
| | | CUCG | AAAUAGCAAGU | mUmAmGmCAAGUUAAA | |
| | | | UAAAAUAAGGC | AUAAGGCUAGUCCGUUA | |
| | | | UAGUCCGUUAU | UCAmAmCmUmUmGmAm | |
| | | | CAACUUGAAAA | AmAmAmAmGmUmGmGm | |
| | | | AGUGGCACCGA | CmAmCmCmGmAmGmUm | |
| | | | GUCGGUGCUUU | CmGmGmUmGmCmU*mU* | |
| | | | U | mU*mU | |
| G013565 | 262 | GGAUCC | GGAUCCUGUGU | mG*mG*mA*UCCUGUGUC | chr19: |
| | | UGUGUC | CCCCGAGCUGU | CCCGAGCUGUUUUAGAm | 55115850- |
| | | CCCGAG | UUUAGAGCUAG | GmCmUmAmGmAmAmAm | 55115870 |
| | | CU | AAAUAGCAAGU | UmAmGmCAAGUUAAAAU | |
| | | | UAAAAUAAGGC | AAGGCUAGUCCGUUAUC | |
| | | | UAGUCCGUUAU | AmAmCmUmUmGmAmAm | |
| | | | CAACUUGAAAA | AmAmAmGmUmGmGmCm | |
| | | | AGUGGCACCGA | AmCmCmGmAmGmUmCm | |
| | | | GUCGGUGCUUU | GmGmUmGmCmU*mU*mU | |
| | | | U | *mU | |
| G013582 | 263 | CCUGUC | CCUGUCAUGGC | mC*mC*mU*GUCAUGGCA | chr19: |
| | | AUGGCA | AUCUUCCAGGU | UCUUCCAGGUUUUAGAm | 55115951- |
| | | UCUUCC | UUUAGAGCUAG | GmCmUmAmGmAmAmAm | 55115971 |
| | | AG | AAAUAGCAAGU | UmAmGmCAAGUUAAAAU | |
| | | | UAAAAUAAGGC | AAGGCUAGUCCGUUAUC | |
| | | | UAGUCCGUUAU | AmAmCmUmUmGmAmAm | |
| | | | CAACUUGAAAA | AmAmAmGmUmGmGmCm | |
| | | | AGUGGCACCGA | AmCmCmGmAmGmUmCm | |
| | | | GUCGGUGCUUU | GmGmUmGmCmU*mU*mU | |
| | | | U | *mU | |
| G013584 | 264 | CCCUGG | CCCUGGAAGAU | mC*mC*mC*UGGAAGAUG | chr19: |
| | | AAGAU | GCCAUGACAGU | CCAUGACAGUUUUAGAm | 55115949- |
| | | GCCAUG | UUUAGAGCUAG | GmCmUmAmGmAmAmAm | 55115969 |
| | | ACA | AAAUAGCAAGU | UmAmGmCAAGUUAAAAU | |
| | | | UAAAAUAAGGC | AAGGCUAGUCCGUUAUC | |
| | | | UAGUCCGUUAU | AmAmCmUmUmGmAmAm | |
| | | | CAACUUGAAAA | AmAmAmGmUmGmGmCm | |
| | | | AGUGGCACCGA | AmCmCmGmAmGmUmCm | |
| | | | GUCGGUGCUUU | GmGmUmGmCmU*mU*mU | |
| | | | U | *mU | |

As used herein, the terms “mA,” “mC,” “mU,” or “mG” denote a nucleotide that has been modified with 2′-O-Me; “*” denote a PS modification; the terms A*, C*, U*, or G* denote a nucleotide that is linked to the next (e.g., 3′) nucleotide with a PS bond.

In some embodiments, provided herein is a composition comprising: a. a gRNA comprising a guide sequence chosen from: i) SEQ ID NOs: 251-264; ii) at least 17, 18, 19, or 20 contiguous nucleotides of a sequence selected from SEQ ID NOs: 251-264; iii) a guide sequence at least 95%, 90%, or 85% identical to a sequence selected from SEQ ID NOs: 251-264; iv) a sequence that comprises 10 contiguous nucleotides±10 nucleotides of a genomic coordinate listed in Table 5; v) at least 17, 18, 19, or 20 contiguous nucleotides of a sequence from (iv); or vi) a guide sequence that is at least 95%, 90%, or 85% identical to a sequence selected from (v); or b. a nucleic acid encoding a gRNA of (a.).

In some embodiments, provided herein is a method of altering a DNA sequence within an AAVS1 gene, comprising delivering to a cell: a. a gRNA comprising a guide sequence chosen from: i) SEQ ID NOs: 251-264; ii) at least 17, 18, 19, or 20 contiguous nucleotides of a sequence selected from SEQ ID NOs: 251-264; iii) a guide sequence at least 95%, 90%, or 85% identical to a sequence selected from SEQ ID NOs: 251-264; iv) a sequence that comprises 10 contiguous nucleotides±10 nucleotides of a genomic coordinate listed in Table 5; v) at least 17, 18, 19, or 20 contiguous nucleotides of a sequence from (iv); or vi) a guide sequence that is at least 95%, 90%, or 85% identical to a sequence selected from (v); or b. a nucleic acid encoding a gRNA of (a.).

In some embodiments, provided herein is method of immunotherapy comprising administering a composition comprising an engineered cell to a subject, wherein the cell comprises a genomic modification in the AAVS1 gene, wherein the genetic modification comprises an insertion within the genomic coordinates selected from: chr19:55115695-55115715; chr19:55115588-55115608; chr19:55115616-55115636; chr19:55115623-55115643; chr19:55115637-55115657; chr19:55115691-55115711; chr19:55115755-55115775; chr19:55115823-55115843; chr19:55115834-55115854; chr19:55115835-55115855; chr19:55115836-55115856; chr19:55115850-55115870; chr19:55115951-55115971; and chr19:55115949-55115969; or wherein the cell is engineered by delivering to the cell: a. a gRNA comprising a guide sequence chosen from: i) SEQ ID NOs: 251-264; ii) at least 17, 18, 19, or 20 contiguous nucleotides of a sequence selected from SEQ ID NOs: 251-264; iii) a guide sequence at least 95%, 90%, or 85% identical to a sequence selected from SEQ ID NOs: 251-264; iv) a sequence that comprises 10 contiguous nucleotides±10 nucleotides of a genomic coordinate listed in Table 5; v) at least 17, 18, 19, or 20 contiguous nucleotides of a sequence from (iv); or vi) a guide sequence that is at least 95%, 90%, or 85% identical to a sequence selected from (v); or b. a nucleic acid encoding a gRNA of (a.).

In some embodiments, provided herein is an engineered cell comprising a genetic modification in the AAVS1 gene, wherein the genetic modification comprises an insertion within the genomic coordinates chosen from: chr19:55115695-55115715; chr19:55115588-55115608; chr19:55115616-55115636; chr19:55115623-55115643; chr19:55115637-55115657; chr19:55115691-55115711; chr19:55115755-55115775; chr19:55115823-55115843; chr19:55115834-55115854; chr19:55115835-55115855; chr19:55115836-55115856; chr19:55115850-55115870; chr19:55115951-55115971; and chr19:55115949-55115969.

### D. Donor Nucleic Acid

The compositions and methods disclosed herein may include a donor nucleic acid, i.e., a template nucleic acid, encoding an exogenous gene. The donor/template nucleic acid may be used to alter or insert the exogenous gene at or near a target site for a Cas nuclease, such as at a genetic locus. In some embodiments, the methods comprise introducing a template to the cell. In some embodiments, a single template may be provided.

In other embodiments, two or more templates may be provided such that editing may occur at two or more target sites. For example, different templates may be provided to edit a single gene in a cell, or two different genes in a cell. In some embodiments, the compositions and methods disclosed herein include a template nucleic acid encoding an exogenous gene for insertion into the TRAC, AAVS1, or CIITA locus.

In some embodiments, the template may be used in homologous recombination. In some embodiments, the homologous recombination may result in the integration of the template sequence or a portion of the template sequence into a target sequence. In other embodiments, the template may be used in homology-directed repair, which involves DNA strand invasion at the site of the cleavage in a target sequence. In some embodiments, the homology-directed repair may result in including the template sequence in an edited target sequence. In yet other embodiments, the template may be used in gene editing mediated by non-homologous end joining. In some embodiments, the template sequence has no similarity to a target sequence near the cleavage site. In some embodiments, the template or a portion of the template sequence is incorporated. In some embodiments, the template includes flanking inverted terminal repeat (ITR) sequences.

In some embodiments, the template may comprise a first homology arm and a second homology arm (also called a first and second nucleotide sequence) that are complementary to sequences located upstream and downstream of the cleavage site, respectively. Where a template contains two homology arms, each arm can be the same length or different lengths, and the sequence between the homology arms can be substantially similar or identical to the target sequence between the homology arms, or it can be entirely unrelated. In some embodiments, the degree of complementarity or percent identity between a first nucleotide sequence on the template and the sequence upstream of the cleavage site, and between a second nucleotide sequence on the template and the sequence downstream of the cleavage site, may permit homologous recombination, such as, e.g., high-fidelity homologous recombination, between the template and the target nucleic acid molecule. In some embodiments, the degree of complementarity may be about 50%, 55%, 60%, 65%, 70%, 75%, 80%, 85%, 90%, 95%, 97%, 98%, 99%, or 100%. In some embodiments, the degree of complementarity may be about 95%, 97%, 98%, 99%, or 100%. In some embodiments, the degree of complementarity may be at least 98%, 99%, or 100%. In some embodiments, the degree of complementarity may be 100%. In some embodiments, the percent identity may be about 50%, 55%, 60%, 65%, 70%, 75%, 80%, 85%, 90%, 95%, 97%, 98%, 99%, or 100%. In some embodiments, the percent identity may be about 95%, 97%, 98%, 99%, or 100%. In some embodiments, the percent identity may be at least 98%, 99%, or 100%. In some embodiments, the percent identity may be 100%.

In some embodiments, the template sequence may correspond to, comprise, or consist of an endogenous sequence of a target cell. It may also or alternatively correspond to, comprise, or consist of an exogenous sequence of a target cell. As used herein, the term “endogenous sequence” refers to a sequence that is native to the cell. The term “exogenous sequence” refers to a sequence that is not native to a cell, or a sequence whose native location in the genome of the cell is in a different location. In some embodiments, the endogenous sequence may be a genomic sequence of the cell. In some embodiments, the endogenous sequence may be a chromosomal or extrachromosomal sequence. In some embodiments, the endogenous sequence may be a plasmid sequence of the cell. In some embodiments, the template sequence may be substantially identical to a portion of the endogenous sequence in a cell at or near the cleavage site, but comprise at least one nucleotide change. In some embodiments, editing the cleaved target sequence with the template may result in a mutation comprising an insertion, deletion, or substitution of one or more nucleotides of the target sequence. In some embodiments, the mutation may result in one or more amino acid changes in a protein expressed from a gene comprising the target sequence.

In some embodiments, the mutation may result in one or more nucleotide changes in an RNA expressed from the target insertion site. In some embodiments, the mutation may alter the expression level of a target gene. In some embodiments, the mutation may result in increased or decreased expression of the target gene. In some embodiments, the mutation may result in gene knock-down. In some embodiments, the mutation may result in gene knock-out. In some embodiments, the mutation may result in restored gene function. In some embodiments, editing of the cleaved target nucleic acid molecule with the template may result in a change in an exon sequence, an intron sequence, a regulatory sequence, a transcriptional control sequence, a translational control sequence, a splicing site, or a non-coding sequence of the target nucleic acid molecule, such as DNA.

In other embodiments, the template sequence may comprise an exogenous sequence. In some embodiments, the exogenous sequence may comprise a coding sequence. In some embodiments, the exogenous sequence may comprise a protein or RNA coding sequence (e.g., an ORF) operably linked to an exogenous promoter sequence such that, upon integration of the exogenous sequence into the target sequence, the cell is capable of expressing the protein or RNA encoded by the integrated sequence. In other embodiments, upon integration of the exogenous sequence into the target nucleic acid molecule, the expression of the integrated sequence may be regulated by an endogenous promoter sequence. In some embodiments, the exogenous sequence may provide a cDNA sequence encoding a protein or a portion of the protein. In yet other embodiments, the exogenous sequence may comprise or consist of an exon sequence, an intron sequence, a regulatory sequence, a transcriptional control sequence, a translational control sequence, a splicing site, or a non-coding sequence. In some embodiments, the integration of the exogenous sequence may result in restored gene function. In some embodiments, the integration of the exogenous sequence may result in a gene knock-in. In some embodiments, the integration of the exogenous sequence may result in a gene knock-out.

The template may be of any suitable length. In some embodiments, the template may comprise 10, 15, 20, 25, 50, 75, 100, 150, 200, 500, 1000, 1500, 2000, 2500, 3000, 3500, 4000, 4500, 5000, 5500, 6000, or more nucleotides in length. The template may be a single-stranded nucleic acid. The template can be double-stranded or partially double-stranded nucleic acid. In some embodiments, the single stranded template is 20, 30, 40, 50, 75, 100, 125, 150, 175, or 200 nucleotides in length. In some embodiments, the template may comprise a nucleotide sequence that is complementary to a portion of the target sequence comprising the target sequence (i.e., a “homology arm”). In some embodiments, the template may comprise a homology arm that is complementary to the sequence located upstream or downstream of the cleavage site on the target sequence.

In some embodiments, the template contains ssDNA or dsDNA containing flanking invert-terminal repeat (ITR) sequences. In some embodiments, the template is provided as a vector, plasmid, minicircle, nanocircle, or PCR product.

### VII. LIPID NUCLEIC ACID ASSEMBLIES

The following section provides additional features of lipid-based delivery compositions, including lipid nanoparticles (LNPs) and lipoplexes, for the first genome editing tool, the second genome editing tool, or a nucleic acid encoding the same. In some embodiments, the first genome editing tool, the second genome editing tool, or a nucleic acid encoding the same is delivered to the cell via at least one lipid nanoparticle (LNP). In some embodiments, the first genome editing tool, the second genome editing tool, or a nucleic acid encoding the same is contained in at least one LNP.

In some embodiments, LNP refers to lipid nanoparticles with a diameter of <100 nm, or a population of LNP with an average diameter of <100 nm, as measured by dynamic light scattering. In some embodiments, the particle size is a number average. In some embodiments, the particle size is a Z-average. In certain embodiments, an LNP has a diameter of about 1-250 nm, 10-200 nm, about 20-150 nm, about 35-150 nm, about 50-150 nm, about 50-100 nm, about 50-120 nm, about 60-100 nm, about 75-150 nm, about 75-120 nm, or about 75-100 nm, or a population of the LNP with an average diameter, as measured by dynamic light scattering, of about 10-200 nm, about 20-150 nm, about 35-150 nm, about 50-150 nm, about 50-100 nm, about 50-120 nm, about 60-100 nm, about 75-150 nm, about 75-120 nm, or about 75-100 nm. In preferred embodiments, an LNP composition has a diameter of 75-150 nm.

LNPs are formed by precise mixing a lipid component (e.g., in ethanol) with an aqueous nucleic acid component and LNPs are uniform in size. Lipoplexes are particles formed by bulk mixing the lipid and nucleic acid components and are between about 100 nm and 1 micron in size. In certain embodiments the lipid nucleic acid assemblies are LNPs. As used herein, a “lipid nucleic acid assembly” comprises a plurality of (i.e., more than one) lipid molecules physically associated with each other by intermolecular forces. A lipid nucleic acid assembly may comprise a bioavailable lipid having a pKa value of <7.5 or <7. The lipid nucleic acid assemblies are formed by mixing an aqueous nucleic acid-containing solution with an organic solvent-based lipid solution, e.g., 100% ethanol. Suitable solutions or solvents include or may contain: water, PBS, Tris buffer, NaCl, citrate buffer, ethanol, chloroform, diethylether, cyclohexane, tetrahydrofuran, methanol, isopropanol. A pharmaceutically acceptable buffer may optionally be comprised in a pharmaceutical formulation comprising the lipid nucleic acid assemblies, e.g., for an ex vivo ACT therapy. In some embodiments, the aqueous solution comprises an RNA, such as an mRNA or a gRNA. In some embodiments, the aqueous solution comprises an mRNA encoding an RNA-guided DNA binding agent, such as Cas9.

In some embodiments, the lipid nucleic acid assembly formulations include an “amine lipid” (sometimes herein or elsewhere described as an “ionizable lipid” or a “biodegradable lipid”), together with an optional “helper lipid”, a “neutral lipid”, and a stealth lipid such as a PEG lipid. In some embodiments, the amine lipids or ionizable lipids are cationic depending on the pH.

### A. Amine Lipids

In some embodiments, LNPs comprise an “amine lipid,” which is, for example an ionizable lipid such as Lipid A, or Lipid D or their equivalents, including acetal analogs of Lipid A or Lipid D.

In some embodiments, the amine lipid is Lipid A, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. Lipid A can be depicted as:

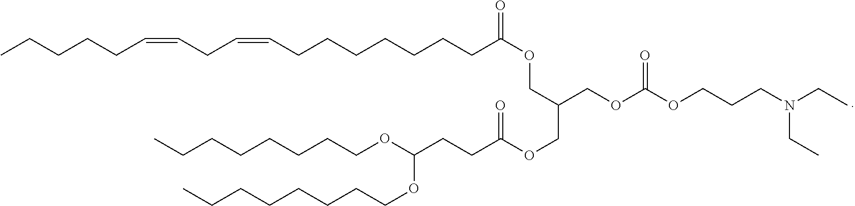 [SMILES: CCCCC/C=C\C/C=C\CCCCCCCC(=O)OCC(COC(=O)CCC(OCCCCCCCC)OCCCCCCCC)COC(=O)OCCCN(CC)CC]

Lipid A may be synthesized according to WO2015/095340 (e.g., pp. 84-86). In some embodiments, the amine lipid is Lipid A, or an amine lipid provided in WO2020/219876, which is hereby incorporated by reference.

In some embodiments, an amine lipid is an analog of Lipid A. In some embodiments, a Lipid A analog is an acetal analog of Lipid A. In particular LNPs, the acetal analog is a C4-C12 acetal analog. In some embodiments, the acetal analog is a C5-C12 acetal analog. In additional embodiments, the acetal analog is a C5-C10 acetal analog. In further embodiments, the acetal analog is chosen from a C4, C5, C6, C7, C9, C10, C11, and C12 acetal analog.

In some embodiments, the amine lipid is a compound having a structure of Formula IA

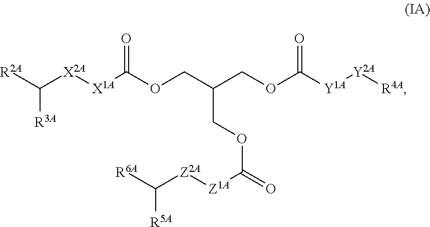

wherein
X1A is O, NH, or a direct bond;
X2A is C2-3 alkylene;
R3A is C1-3 alkyl;
R2A is C1-3 alkyl, or
R2A taken together with the nitrogen atom to which it is attached and 2-3 carbon atoms of X2A form a 5- or 6-membered ring, or
R2A taken together with R3A and the nitrogen atom to which they are attached form a 5-membered ring;
Y1A is C6-10 alkylene;
Y2A is selected from

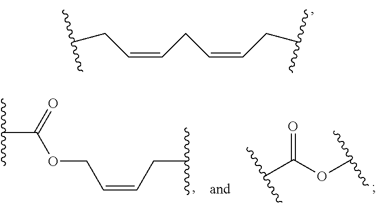 [SMILES: *C/C=C\C/C=C\C* ; *C/C=C\COC(*)=O ; *OC(*)=O]

R4A is C4-11 alkyl;
Z1A is C2-5 alkylene;
Z2A is

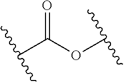 [SMILES: *OC(*)=O]

or absent;
R5A is C6-8 alkyl or C6-8 alkoxy; and
R6A is C6-8 alkyl or C6-8 alkoxy
or a salt thereof.

In some embodiments, the amine lipid is a compound of Formula (IIA)

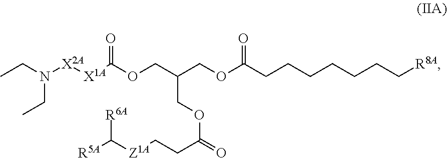

wherein
X1A is O, NH, or a direct bond;
X2A is C2-3 alkylene;
Z1A is C3 alkylene and R5A and R6A are each C6 alkyl, or Z1A is a direct bond and R5A and R6A are each C8 alkoxy; and
R8A is

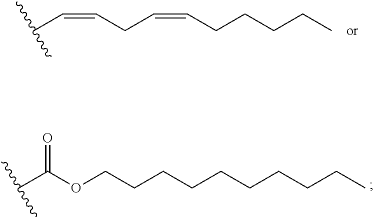 [SMILES: */C=C\C/C=C\CCCCC ; *C(=O)OCCCCCCCCCC]

or a salt thereof.

In certain embodiments, X1A is O. In other embodiments, X1A is NH. In still other embodiments, X1A is a direct bond.

In certain embodiments, X2A is C3 alkylene. In particular embodiments, X2A is C2 alkylene.

In certain embodiments, Z1A is a direct bond and R5A and R6A are each C8 alkoxy. In other embodiments, Z1A is C3 alkylene and R5A and R6A are each C6 alkyl.

In certain embodiments, R8A is

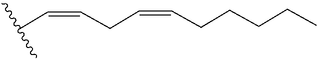 [SMILES: */C=C\C/C=C\CCCCC]

In other embodiments, R8A is

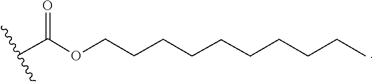 [SMILES: *C(=O)OCCCCCCCCCC]

In certain embodiments, the amine lipid is a salt.

Representative compounds of Formula (IA) include:

| Com- | |
|---|---|
| pound | |
| Num- | |
| ber | Compound |
| 1A | 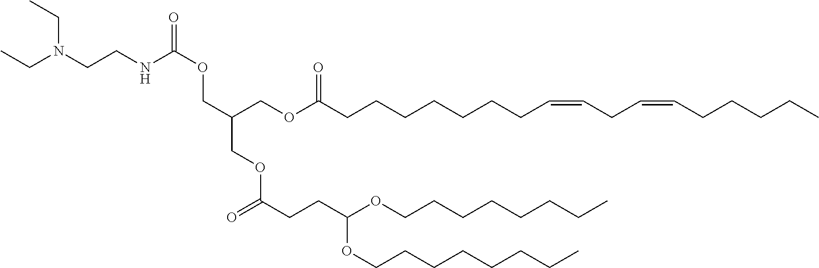 [SMILES: CCCCC/C=C\C/C=C\CCCCCCCC(=O)OCC(COC(=O)CCC(OCCCCCCCC)OCCCCCCCC)COC(=O)NCCN(CC)CC] |
| 2A | 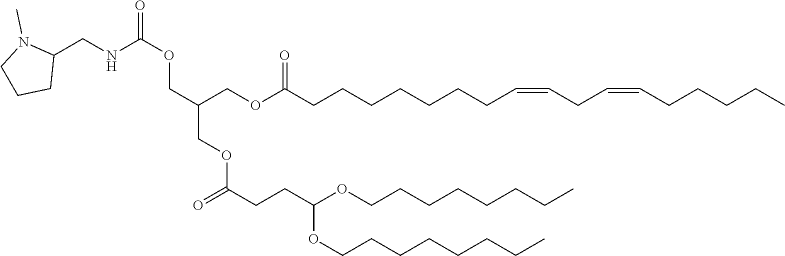 [SMILES: CCCCC/C=C\C/C=C\CCCCCCCC(=O)OCC(COC(=O)CCC(OCCCCCCCC)OCCCCCCCC)COC(=O)NCC1CCCN1C] |
| 3A | 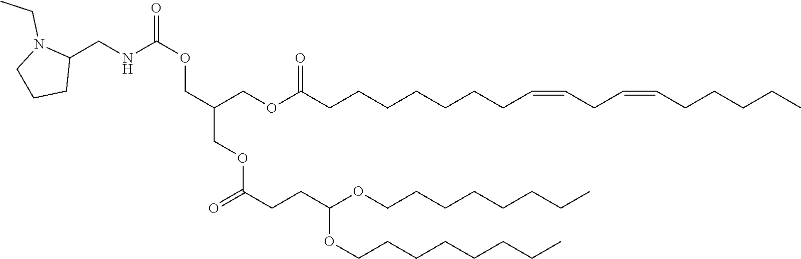 [SMILES: CCCCC/C=C\C/C=C\CCCCCCCC(=O)OCC(COC(=O)CCC(OCCCCCCCC)OCCCCCCCC)COC(=O)NCC1CCCN1CC] |
| 4A | 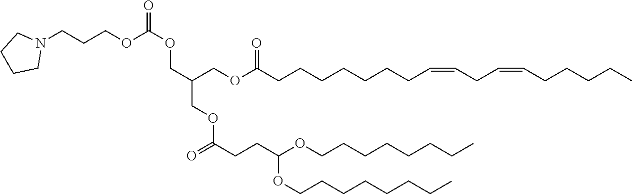 [SMILES: CCCCC/C=C\C/C=C\CCCCCCCC(=O)OCC(COC(=O)CCC(OCCCCCCCC)OCCCCCCCC)COC(=O)OCCCN1CCCC1] |
| 5A | 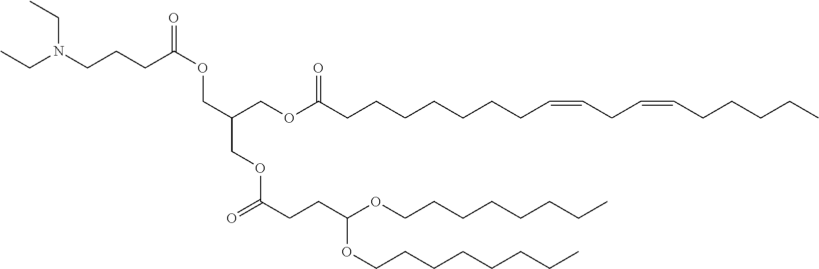 [SMILES: CCCCC/C=C\C/C=C\CCCCCCCC(=O)OCC(COC(=O)CCCN(CC)CC)COC(=O)CCC(OCCCCCCCC)OCCCCCCCC] |
| 6A | 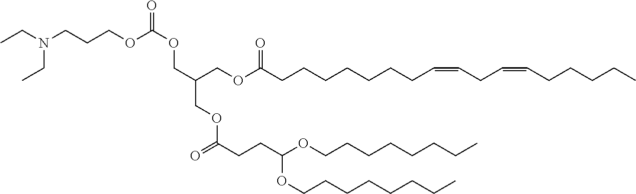 [SMILES: CCCCC/C=C\C/C=C\CCCCCCCC(=O)OCC(COC(=O)CCC(OCCCCCCCC)OCCCCCCCC)COC(=O)OCCCN(CC)CC] |
| 7A | 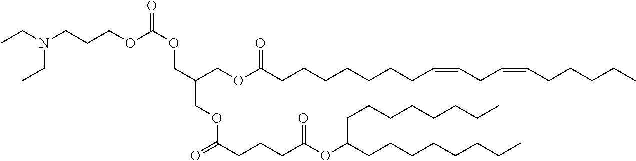 [SMILES: CCCCC/C=C\C/C=C\CCCCCCCC(=O)OCC(COC(=O)CCCC(=O)OC(CCCCCCCC)CCCCCCCC)COC(=O)OCCCN(CC)CC] |
| 8A | 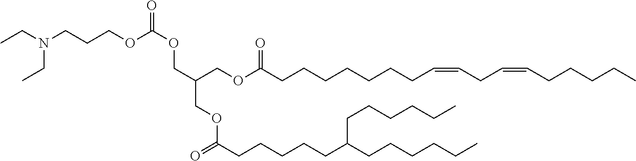 [SMILES: CCCCC/C=C\C/C=C\CCCCCCCC(=O)OCC(COC(=O)CCCCCC(CCCCCC)CCCCCC)COC(=O)OCCCN(CC)CC] |
| 9A | 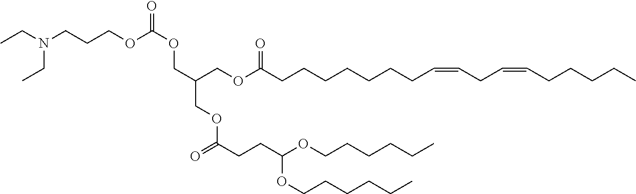 [SMILES: CCCCC/C=C\C/C=C\CCCCCCCC(=O)OCC(COC(=O)CCC(OCCCCCC)OCCCCCC)COC(=O)OCCCN(CC)CC] |
| 10A | 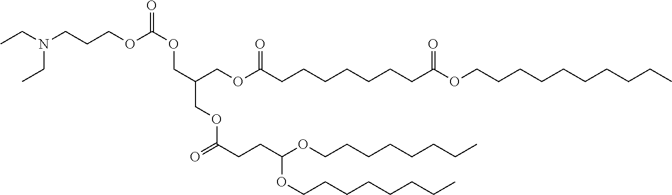 [SMILES: CCCCCCCCCCOC(=O)CCCCCCCC(=O)OCC(COC(=O)CCC(OCCCCCCCC)OCCCCCCCC)COC(=O)OCCCN(CC)CC] |
| 11A | 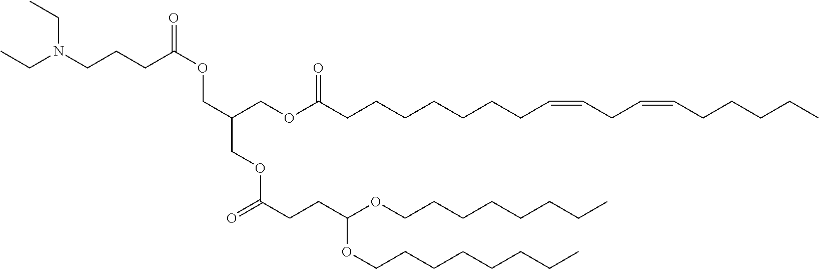 [SMILES: CCCCC/C=C\C/C=C\CCCCCCCC(=O)OCC(COC(=O)CCCN(CC)CC)COC(=O)CCC(OCCCCCCCC)OCCCCCCCC] |
| 12A | 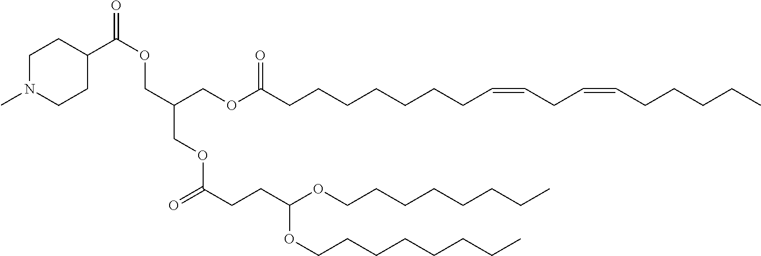 [SMILES: CCCCC/C=C\C/C=C\CCCCCCCC(=O)OCC(COC(=O)CCC(OCCCCCCCC)OCCCCCCCC)COC(=O)C1CCN(C)CC1] |
| 13A | 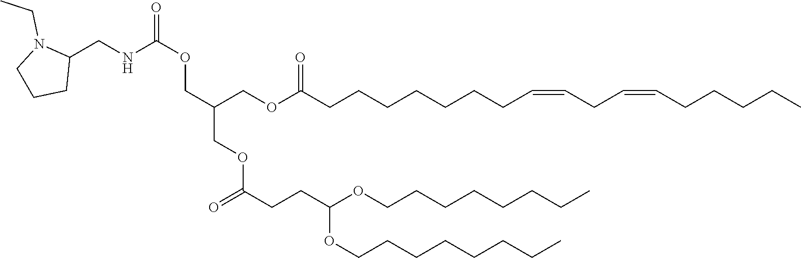 [SMILES: CCCCC/C=C\C/C=C\CCCCCCCC(=O)OCC(COC(=O)CCC(OCCCCCCCC)OCCCCCCCC)COC(=O)NCC1CCCN1CC] |
| 14A | 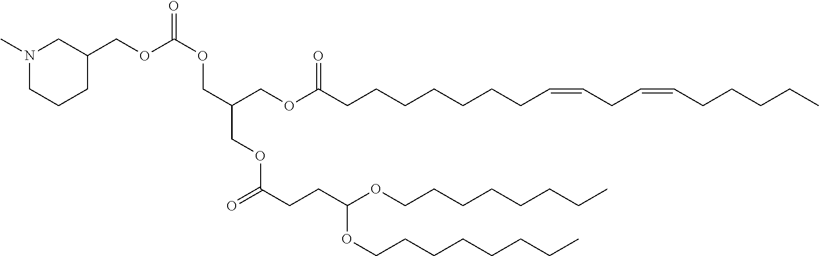 [SMILES: CCCCC/C=C\C/C=C\CCCCCCCC(=O)OCC(COC(=O)CCC(OCCCCCCCC)OCCCCCCCC)COC(=O)OCC1CCCN(C)C1] |
| 15A | 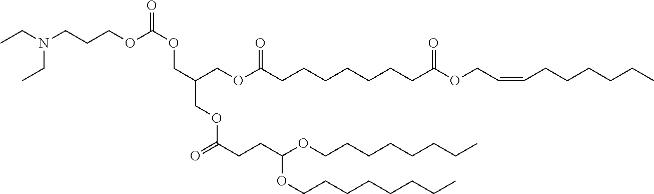 [SMILES: CCCCCC/C=C\COC(=O)CCCCCCCC(=O)OCC(COC(=O)CCC(OCCCCCCCC)OCCCCCCCC)COC(=O)OCCCN(CC)CC] |
| 16A | 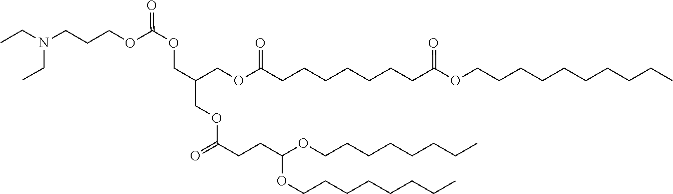 [SMILES: CCCCCCCCCCOC(=O)CCCCCCCC(=O)OCC(COC(=O)CCC(OCCCCCCCC)OCCCCCCCC)COC(=O)OCCCN(CC)CC] |
| 17A | 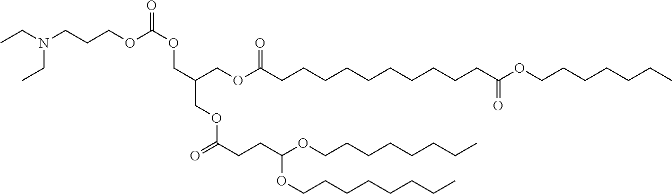 [SMILES: CCCCCCCCOC(CCC(=O)OCC(COC(=O)CCCCCCCCCCC(=O)OCCCCCCC)COC(=O)OCCCN(CC)CC)OCCCCCCCC] |
| 18A | 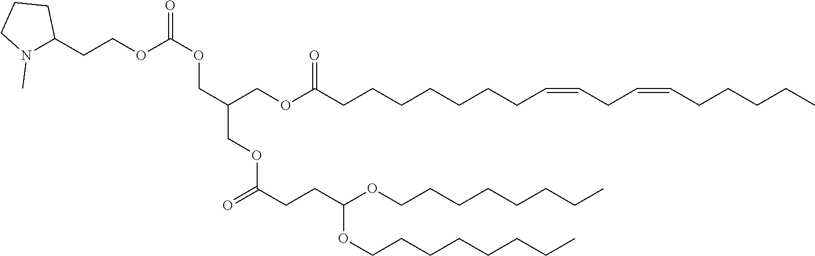 [SMILES: CCCCC/C=C\C/C=C\CCCCCCCC(=O)OCC(COC(=O)CCC(OCCCCCCCC)OCCCCCCCC)COC(=O)OCCC1CCCN1C] |
| 19A | 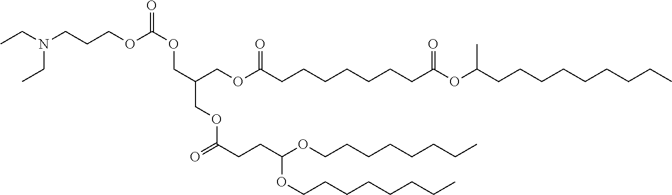 [SMILES: CCCCCCCCCC(C)OC(=O)CCCCCCCC(=O)OCC(COC(=O)CCC(OCCCCCCCC)OCCCCCCCC)COC(=O)OCCCN(CC)CC] |

or a salt thereof, such as a pharmaceutically acceptable salt thereof.

In some embodiments, the amine lipid is Lipid D, which is nonyl 8-((7,7-bis(octyloxy)heptyl)(2-hydroxyethyl)amino)octanoate:

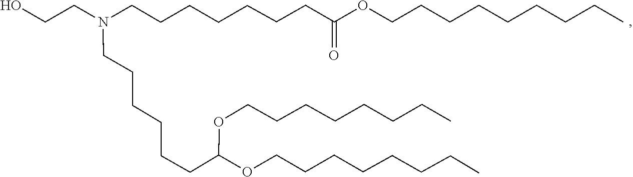 [SMILES: CCCCCCCCCOC(=O)CCCCCCCN(CCO)CCCCCCC(OCCCCCCCC)OCCCCCCCC]

or a salt thereof.

Lipid D may be synthesized according to WO2020072605 and Mol. Ther. 2018, 26(6), 1509-1519 (“Sabnis”), which are incorporated by reference in their entireties. In some embodiments, the amine lipid Lipid D, or an amine lipid provided in WO2020072605, which is hereby incorporated by reference.

In some embodiments, the amine lipid is a compound having a structure of Formula IB:

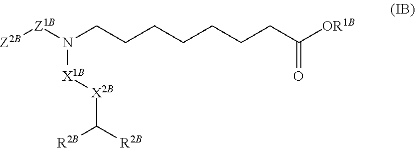

wherein
X^{1B} is C₆₋₇ alkylene;
X^{2B} is

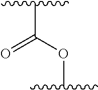 [SMILES: *OC(*)=O]

or absent, provided that if X^{2B} is

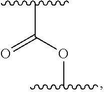 [SMILES: *OC(*)=O]

R^{2B} is not alkoxy;
Z^{1B} is C₂₋₃ alkylene;
Z^{2B} is selected from —OH, —NHC(═O)OCH₃, and —NHS(═O)₂CH₃;
R^{1B} is C₇₋₉ unbranched alkyl; and
each R^{2B} is independently C₈ alkyl or C₈ alkoxy;
or a salt thereof

In some embodiments, the amine lipid is a compound of Formula (IIB)

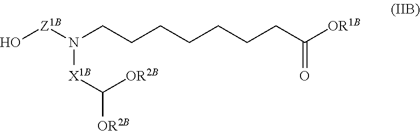

wherein
X^{1B} is C₆₋₇ alkylene;
Z^{1B} is C₂₋₃ alkylene;
R^{1B} is C₇₋₉ unbranched alkyl; and
each R^{2B} is C₈ alkyl;
or a salt thereof.

In certain embodiments, X^{1B} is C6 alkylene. In other embodiments, X^{1B} is C₇ alkylene.

In certain embodiments, Z^{1B} is a direct bond and R^{5B} and R^{6B} are each C₈ alkoxy. In other embodiments, Z^{1B} is C₃ alkylene and R^{5B} and R^{6B} are each C₆ alkyl.

In certain embodiments, X^{2B} is

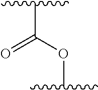 [SMILES: *OC(*)=O]

and R^{2B} is not alkoxy. In other embodiments, X^{2B} is absent.

In certain embodiments, Z^{1B} is C₂ alkylene; In other embodiments, Z^{1B} is C₃ alkylene.

In certain embodiments, Z^{2B} is —OH. In other embodiments, Z^{2B} is —NHC(═O)OCH₃. In other embodiments, Z^{2B} is —NHS(═O)₂CH₃.

In certain embodiments, R^{1B} is C₇ unbranched alkylene. In other embodiments, R^{1B} is C₈ branched or unbranched alkylene. In other embodiments, R^{1B} is C₉ branched or unbranched alkylene.

In certain embodiments, the amine lipid is a salt.

Representative compounds of Formula (IB) include:

| Compound | |
|---|---|
| Number | Compound |
| 1B | 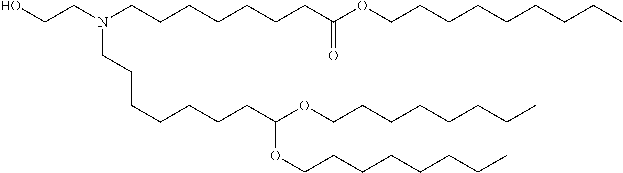 [SMILES: CCCCCCCCCOC(=O)CCCCCCCN(CCO)CCCCCCCC(OCCCCCCCC)OCCCCCCCC] |
| 2B | 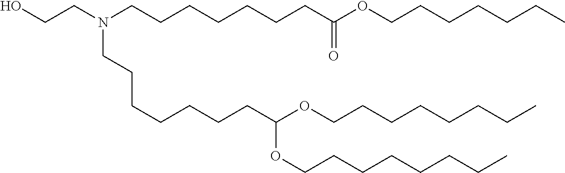 [SMILES: CCCCCCCCOC(CCCCCCCN(CCO)CCCCCCCC(=O)OCCCCCCC)OCCCCCCCC] |
| 3B | 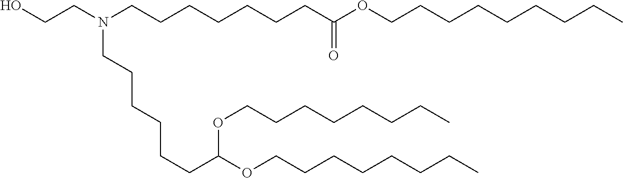 [SMILES: CCCCCCCCCOC(=O)CCCCCCCN(CCO)CCCCCCC(OCCCCCCCC)OCCCCCCCC] |
| 4B | 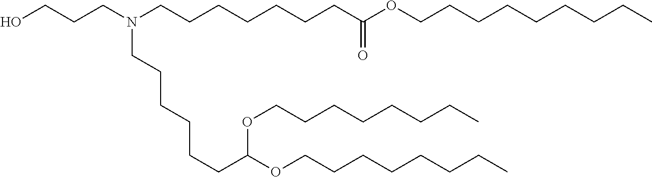 [SMILES: CCCCCCCCCOC(=O)CCCCCCCN(CCCO)CCCCCCC(OCCCCCCCC)OCCCCCCCC] |
| 5B | 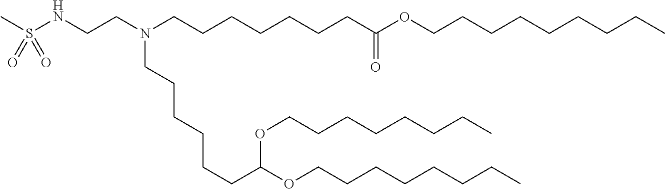 [SMILES: CCCCCCCCCOC(=O)CCCCCCCN(CCCCCCC(OCCCCCCCC)OCCCCCCCC)CCNS(C)(=O)=O] |
| 6B | 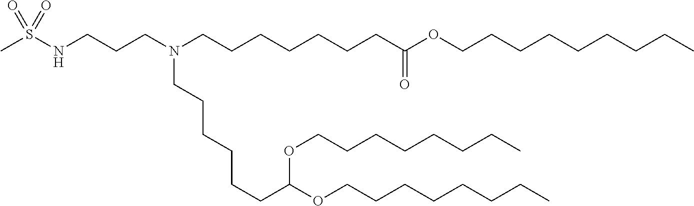 [SMILES: CCCCCCCCCOC(=O)CCCCCCCN(CCCCCCC(OCCCCCCCC)OCCCCCCCC)CCCNS(C)(=O)=O] |
| 7B | 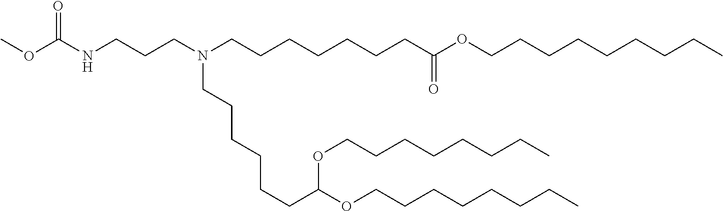 [SMILES: CCCCCCCCCOC(=O)CCCCCCCN(CCCCCCC(OCCCCCCCC)OCCCCCCCC)CCCNC(=O)OC] |

or a salt thereof, such as a pharmaceutically acceptable salt thereof.

Amine lipids and other “biodegradable lipids” suitable for use in the lipid nucleic acid assemblies described herein are biodegradable in vivo or ex vivo. The amine lipids have low toxicity (e.g., are tolerated in animal models without adverse effect in amounts of greater than or equal to 10 mg/kg). In some embodiments, lipid nucleic acid assemblies comprising an amine lipid include those where at least 75% of the amine lipid is cleared from the plasma or the engineered cell within 8, 10, 12, 24, or 48 hours, or 3, 4, 5, 6, 7, or 10 days. In some embodiments, lipid nucleic acid assemblies comprising an amine lipid include those where at least 50% of the nucleic acid, e.g., mRNA or gRNA, is cleared from the plasma within 8, 10, 12, 24, or 48 hours, or 3, 4, 5, 6, 7, or 10 days. In some embodiments, lipid nucleic acid assemblies comprising an amine lipid include those where at least 50% of the lipid nucleic acid assembly is cleared from the plasma within 8, 10, 12, 24, or 48 hours, or 3, 4, 5, 6, 7, or 10 days, for example by measuring a lipid (e.g., an amine lipid), nucleic acid, e.g., RNA/mRNA, or other component. In some embodiments, lipid-encapsulated versus free lipid, RNA, or nucleic acid component of the lipid nucleic acid assembly is measured.

Biodegradable lipids include, for example the biodegradable lipids of WO 2020/219876 (e.g., at pp. 13-33, 66-87), WO 2020/118041, WO 2020/072605 (e.g., at pp. 5-12, 21-29, 61-68, WO 2019/067992, WO 2017/173054, WO 2015/095340, and WO 2014/136086, and LNPs include LNP compositions described therein, the lipids and compositions of which are hereby incorporated by reference.

Lipid clearance may be measured as described in literature. See Maier, M. A., et al. Biodegradable Lipids Enabling Rapidly Eliminated Lipid Nanoparticles for Systemic Delivery of RNAi Therapeutics. Mol. Ther. 2013, 21(8), 1570-78 (“Maier”). For example, in Maier, LNP-siRNA systems containing luciferases-targeting siRNA were administered to six- to eight-week old male C57Bl/6 mice at 0.3 mg/kg by intravenous bolus injection via the lateral tail vein. Blood, liver, and spleen samples were collected at 0.083, 0.25, 0.5, 1, 2, 4, 8, 24, 48, 96, and 168 hours post-dose. Mice were perfused with saline before tissue collection and blood samples were processed to obtain plasma. All samples were processed and analyzed by LC-MS. Further, Maier describes a procedure for assessing toxicity after administration of LNP-siRNA formulations. For example, a luciferase-targeting siRNA was administered at 0, 1, 3, 5, and 10 mg/kg (5 animals/group) via single intravenous bolus injection at a dose volume of 5 mL/kg to male Sprague-Dawley rats. After 24 hours, about 1 mL of blood was obtained from the jugular vein of conscious animals and the serum was isolated. At 72 hours post-dose, all animals were euthanized for necropsy. Assessments of clinical signs, body weight, serum chemistry, organ weights and histopathology were performed. Although Maier describes methods for assessing siRNA-LNP formulations, these methods may be applied to assess clearance, pharmacokinetics, and toxicity of administration of LNPs of the present disclosure.

Ionizable and bioavailable lipids for LNP delivery of nucleic acids known in the art are suitable. Lipids may be ionizable depending upon the pH of the medium they are in. For example, in a slightly acidic medium, the lipid, such as an amine lipid, may be protonated and thus bear a positive charge. Conversely, in a slightly basic medium, such as, for example, blood where pH is approximately 7.35, the lipid, such as an amine lipid, may not be protonated and thus bear no charge.

The ability of a lipid to bear a charge is related to its intrinsic pKa. In some embodiments, the amine lipids of the present disclosure may each, independently, have a pKa in the range of from about 5.1 to about 7.4. In some embodiments, the bioavailable lipids of the present disclosure may each, independently, have a pKa in the range of from about 5.1 to about 7.4, such as from about 5.5 to about 6.6, from about 5.6 to about 6.4, from about 5.8 to about 6.2, or from about 5.8 to about 6.5. For example, the amine lipids of the present disclosure may each, independently, have a pKa in the range of from about 5.8 to about 6.5. Lipids with a pKa ranging from about 5.1 to about 7.4 are effective for delivery of cargo in vivo, e.g. to the liver. Further, it has been found that lipids with a pKa ranging from about 5.3 to about 6.4 are effective for delivery in vivo, e.g. to tumors. See, e.g., WO2014/136086.

### B. Additional Lipids

“Neutral lipids” suitable for use in a lipid composition of the disclosure include, for example, a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine (DSPC), pohsphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In one embodiment, the neutral phospholipid may be selected from the group consisting of distearoylphosphatidylcholine (DSPC) and dimyristoyl phosphatidyl ethanolamine (DMPE). In another embodiment, the neutral phospholipid may be distearoylphosphatidylcholine (DSPC).

“Helper lipids” include steroids, sterols, and alkyl resorcinols. Helper lipids suitable for use in the present disclosure include, but are not limited to, cholesterol, 5-heptadecylresorcinol, and cholesterol hemisuccinate. In one embodiment, the helper lipid may be cholesterol. In one embodiment, the helper lipid may be cholesterol hemisuccinate.

“Stealth lipids” are lipids that alter the length of time the nanoparticles can exist in vivo (e.g., in the blood). Stealth lipids may assist in the formulation process by, for example, reducing particle aggregation and controlling particle size. Stealth lipids used herein may modulate pharmacokinetic properties of the lipid nucleic acid assembly or aid in stability of the nanoparticle ex vivo. Stealth lipids suitable for use in a lipid composition of the disclosure include, but are not limited to, stealth lipids having a hydrophilic head group linked to a lipid moiety. Stealth lipids suitable for use in a lipid composition of the present disclosure and information about the biochemistry of such lipids can be found in Romberg et al., Pharmaceutical Research, Vol. 25, No. 1, 2008, pg. 55-71 and Hoekstra et al., Biochimica et Biophysica Acta 1660 (2004) 41-52. Additional suitable PEG lipids are disclosed, e.g., in WO 2006/007712.

In one embodiment, the hydrophilic head group of stealth lipid comprises a polymer moiety selected from polymers based on PEG. Stealth lipids may comprise a lipid moiety. In some embodiments, the stealth lipid is a PEG lipid.

In one embodiment, a stealth lipid comprises a polymer moiety selected from polymers based on PEG (sometimes referred to as poly(ethylene oxide)), poly(oxazoline), poly(vinyl alcohol), poly(glycerol), poly(N-vinylpyrrolidone), polyaminoacids and poly[N-(2-hydroxypropyl)methacrylamide].

In one embodiment, the PEG lipid comprises a polymer moiety based on PEG (sometimes referred to as poly(ethylene oxide)).

The PEG lipid further comprises a lipid moiety. In some embodiments, the lipid moiety may be derived from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. In some embodiments, the alkyl chain length comprises about C10 to C20. The dialkylglycerol or dialkylglycamide group can further comprise one or more substituted alkyl groups. The chain lengths may be symmetrical or asymmetrical.

Unless otherwise indicated, the term “PEG” as used herein means any polyethylene glycol or other polyalkylene ether polymer. In one embodiment, PEG is an optionally substituted linear or branched polymer of ethylene glycol or ethylene oxide. In one embodiment, PEG is unsubstituted. In one embodiment, the PEG is substituted, e.g., by one or more alkyl, alkoxy, acyl, hydroxy, or aryl groups. In one embodiment, the term includes PEG copolymers such as PEG-polyurethane or PEG-polypropylene (see, e.g., J. Milton Harris, Poly(ethylene glycol) chemistry: biotechnical and biomedical applications (1992)); in another embodiment, the term does not include PEG copolymers. In one embodiment, the PEG has a molecular weight of from about 130 to about 50,000, in a sub-embodiment, about 150 to about 30,000, in a sub-embodiment, about 150 to about 20,000, in a sub-embodiment about 150 to about 15,000, in a sub-embodiment, about 150 to about 10,000, in a sub-embodiment, about 150 to about 6,000, in a sub-embodiment, about 150 to about 5,000, in a sub-embodiment, about 150 to about 4,000, in a sub-embodiment, about 150 to about 3,000, in a sub-embodiment, about 300 to about 3,000, in a sub-embodiment, about 1,000 to about 3,000, and in a sub-embodiment, about 1,500 to about 2,500.

In some embodiments, the PEG (e.g., conjugated to a lipid moiety or lipid, such as a stealth lipid), is a “PEG-2K,” also termed “PEG 2000,” which has an average molecular weight of about 2,000 Daltons. PEG-2K is represented herein by the following formula (IV), wherein n is 45, meaning that the number averaged degree of polymerization comprises about 45 subunits

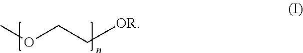

However, other PEG embodiments known in the art may be used, including, e.g., those where the number-averaged degree of polymerization comprises about 23 subunits (n=23), or 68 subunits (n=68). In some embodiments, n may range from about 30 to about 60. In some embodiments, n may range from about 35 to about 55. In some embodiments, n may range from about 40 to about 50. In some embodiments, n may range from about 42 to about 48. In some embodiments, n may be 45. In some embodiments, R may be selected from H, substituted alkyl, and unsubstituted alkyl. In some embodiments, R may be unsubstituted alkyl. In some embodiments, R may be methyl.

In any of the embodiments described herein, the PEG lipid may be selected from PEG-dilauroylglycerol, PEG-dimyristoylglycerol (PEG-DMG catalog #GM-020 from NOF, Tokyo, Japan), such as e.g., 1,2-dimyristoyl-rac-glycero-3-methylpolyoxyethylene glycol 2000 (PEG2k-DMG), PEG-dipalmitoylglycerol, PEG-distearoylglycerol (PEG-DSPE) (catalog #DSPE-020CN, NOF, Tokyo, Japan), PEG-dilaurylglycamide, PEG-dimyristylglycamide, PEG-dipalmitoylglycamide, and PEG-distearoylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol)ether), 1,2-dimyristoyl-sn-glycero-3-phosphoethanolamine-N-[methoxy(polyethylene glycol)-2000](PEG2k-DMPE) (cat. #880150P from Avanti Polar Lipids, Alabaster, Alabama, USA), 1,2-distearoyl-sn-glycero-3-phosphoethanolamine-N-[methoxy(polyethylene glycol)-2000](PEG2k-DSPE) (cat. #880120C from Avanti Polar Lipids, Alabaster, Alabama, USA), 1,2-distearoyl-sn-glycerol, methoxypolyethylene glycol (PEG2k-DSG; GS-020, NOF Tokyo, Japan), poly(ethylene glycol)-2000-dimethacrylate (PEG2k-DMA), and 1,2-distearyloxypropyl-3-amine-N-[methoxy(polyethylene glycol)-(PEG2k-DSA). In one embodiment, the PEG lipid may be 1,2-dimyristoyl-rac-glycero-3-methylpolyoxyethylene glycol 2000. In one embodiment, the PEG lipid may be PEG2k-DMG. In some embodiments, the PEG lipid may be PEG2k-DSG. In one embodiment, the PEG lipid may be PEG2k-DSPE. In one embodiment, the PEG lipid may be PEG2k-DMA. In one embodiment, the PEG lipid may be PEG2k-C-DMA. In one embodiment, the PEG lipid may be compound S027, disclosed in WO2016/010840 (paragraphs [00240] to [00244]). In one embodiment, the PEG lipid may be PEG2k-DSA. In one embodiment, the PEG lipid may be PEG2k-C11. In some embodiments, the PEG lipid may be PEG2k-C14. In some embodiments, the PEG lipid may be PEG2k-C16. In some embodiments, the PEG lipid may be PEG2k-C18.

### C. Lipid Nanoparticles (LNPs)

The LNP may contain (i) a biodegradable lipid, (ii) an optional neutral lipid, (iii) a helper lipid, and (iv) a stealth lipid, such as a PEG lipid. The lipid nucleic acid assembly may contain a biodegradable lipid and one or more of a neutral lipid, a helper lipid, and a stealth lipid, such as a PEG lipid.

The lipid nucleic acid assembly may contain (i) an amine lipid for encapsulation and for endosomal escape, (ii) a neutral lipid for stabilization, (iii) a helper lipid, also for stabilization, and (iv) a stealth lipid, such as a PEG lipid. The lipid nucleic acid assembly may contain an amine lipid and one or more of a neutral lipid, a helper lipid, also for stabilization, and a stealth lipid, such as a PEG lipid.

An LNP may comprise a nucleic acid, e.g., an RNA, component that includes one or more of an RNA-guided DNA-binding agent, a Cas nuclease mRNA, a Class 2 Cas nuclease mRNA, a Cas9 mRNA, and a gRNA. In some embodiments, a LNP may include a Class 2 Cas nuclease and a gRNA as the RNA component. In some embodiments, n LNP may comprise the RNA component, an amine lipid, a helper lipid, a neutral lipid, and a stealth lipid. In certain LNPs, the helper lipid is cholesterol. In other compositions, the neutral lipid is DSPC. In additional embodiments, the stealth lipid is PEG2k-DMG or PEG2k-C11. In some embodiments, the LNP comprises Lipid A or an equivalent of Lipid A; a helper lipid; a neutral lipid; a stealth lipid; and an RNA such as a gRNA. In some embodiments, the LNP comprises Lipid A or an equivalent of Lipid A; a helper lipid; a stealth lipid; and an RNA such as a gRNA. In some compositions, the amine lipid is Lipid A. In some compositions, the amine lipid is Lipid A or an acetal analog thereof, the helper lipid is cholesterol; the neutral lipid is DSPC; and the stealth lipid is PEG2k-DMG.

In some embodiments, lipid compositions are described according to the respective molar ratios of the component lipids in the formulation. Embodiments of the present disclosure provide lipid compositions described according to the respective molar ratios of the component lipids in the formulation. In one embodiment, the mol % of the amine lipid may be from about 30 mol % to about 60 mol %. In one embodiment, the mol % of the amine lipid may be from about 40 mol % to about 60 mol %. In one embodiment, the mol % of the amine lipid may be from about 45 mol % to about 60 mol %. In one embodiment, the mol % of the amine lipid may be from about 50 mol % to about 60 mol %. In one embodiment, the mol % of the amine lipid may be from about 55 mol % to about 60 mol %. In one embodiment, the mol % of the amine lipid may be from about 50 mol % to about 55 mol %. In one embodiment, the mol % of the amine lipid may be about 50 mol %. In one embodiment, the mol % of the amine lipid may be about 55 mol %. In some embodiments, the amine lipid mol % of the lipid nucleic acid assembly batch will be ±30%, ±25%, ±20%, ±15%, ±10%, ±5%, or ±2.5% of the target mol %. In some embodiments, the amine lipid mol % of the lipid nucleic acid assembly batch will be ±4 mol %, ±3 mol %, ±2 mol %, ±1.5 mol %, ±1 mol %, ±0.5 mol %, or ±0.25 mol % of the target mol %. All mol % numbers are given as a fraction of the lipid component of the LNPs. In some embodiments, lipid nucleic acid assembly inter-lot variability of the amine lipid mol % will be less than 15%, less than 10% or less than 5%.

In one embodiment, the mol % of the neutral lipid may be from about 5 mol % to about 15 mol %. In one embodiment, the mol % of the neutral lipid may be from about 7 mol % to about 12 mol %. In one embodiment, the mol % of the neutral lipid may be about 9 mol %. In some embodiments, the neutral lipid mol % of the lipid nucleic acid assembly batch will be ±30%, ±25%, ±20%, ±15%, ±10%, ±5%, or ±2.5% of the target neutral lipid mol %. In some embodiments, lipid nucleic acid assembly inter-lot variability will be less than 15%, less than 10% or less than 5%.

In one embodiment, the mol % of the helper lipid may be from about 20 mol % to about 60 mol %. In one embodiment, the mol % of the helper lipid may be from about 25 mol % to about 55 mol %. In one embodiment, the mol % of the helper lipid may be from about 25 mol % to about 50 mol %. In one embodiment, the mol % of the helper lipid may be from about 25 mol % to about 40 mol %. In one embodiment, the mol % of the helper lipid may be from about 30 mol % to about 50 mol %. In one embodiment, the mol % of the helper lipid may be from about 30 mol % to about 40 mol %. In one embodiment, the mol % of the helper lipid is adjusted based on amine lipid, neutral lipid, and PEG lipid concentrations to bring the lipid component to 100 mol %. In some embodiments, the helper mol % of the lipid nucleic acid assembly batch will be ±30%, ±25%, ±20%, ±15%, ±10%, ±5%, or ±2.5% of the target mol %. In some embodiments, lipid nucleic acid assembly inter-lot variability will be less than 15%, less than 10% or less than 5%.

In one embodiment, the mol % of the PEG lipid may be from about 1 mol % to about 10 mol %. In one embodiment, the mol % of the PEG lipid may be from about 2 mol % to about 10 mol %. In one embodiment, the mol % of the PEG lipid may be from about 1 mol % to about 3 mol %. In one embodiment, the mol % of the PEG lipid may be from about 2 mol % to about 4 mol %. In one embodiment, the mol % of the PEG lipid may be from about 1.5 mol % to about 2 mol %. In one embodiment, the mol % of the PEG lipid may be from about 2.5 mol % to about 4 mol %. In one embodiment, the mol % of the PEG lipid may be about 3 mol %. In one embodiment, the mol % of the PEG lipid may be about 2.5 mol %. In one embodiment, the mol % of the PEG lipid may be about 2 mol %. In one embodiment, the mol % of the PEG lipid may be about 1.5 mol %. In some embodiments, the PEG lipid mol % of the lipid nucleic acid assembly batch will be ±30%, ±25%, ±20%, ±15%, ±10%, ±5%, or ±2.5% of the target PEG lipid mol %. In some embodiments, LNP, e.g. the LNP composition, inter-lot variability will be less than 15%, less than 10% or less than 5%.

Embodiments of the present disclosure provide LNP compositions, for example, LNP compositions comprising an ionizable lipid (e.g., Lipid A or one of its analogs), a helper lipid, a helper lipid, and a PEG lipid, described according to the respective molar ratios of the component lipids in the formulation. In certain embodiments, the amount of the ionizable lipid is from about 25 mol % to about 45 mol %; the amount of the neutral lipid is from about 10 mol % to about 30 mol %; the amount of the helper lipid is from about 25 mol % to about 65 mol %; and the amount of the PEG lipid is from about 1.5 mol % to about 3.5 mol %. In certain embodiments, the amount of the ionizable lipid is from about 29-44 mol % of the lipid component; the amount of the neutral lipid is from about 11-28 mol % of the lipid component; the amount of the helper lipid is from about 28-55 mol % of the lipid component; and the amount of the PEG lipid is from about 2.3-3.5 mol % of the lipid component. In certain embodiments, the amount of the ionizable lipid is from about 29-38 mol % of the lipid component; the amount of the neutral lipid is from about 11-20 mol % of the lipid component; the amount of the helper lipid is from about 43-55 mol % of the lipid component; and the amount of the PEG lipid is from about 2.3-2.7 mol % of the lipid component. In certain embodiments, the amount of the ionizable lipid is from about 25-34 mol % of the lipid component; the amount of the neutral lipid is from about 10-20 mol % of the lipid component; the amount of the helper lipid is from about 45-65 mol % of the lipid component; and the amount of the PEG lipid is from about 2.5-3.5 mol % of the lipid component. In certain embodiments, the ionizable lipid is about 30-43 mol % of the lipid component; the amount of the neutral lipid is about 10-17 mol % of the lipid component; the amount of the helper lipid is about 43.5-56 mol % of the lipid component; and the amount of the PEG lipid is about 1.5-3 mol % of the lipid component. In certain embodiments, the ionizable lipid is about 33 mol % of the lipid component; the amount of the neutral lipid is about 15 mol % of the lipid component; the amount of the helper lipid is about 49 mol % of the lipid component; and the amount of the PEG lipid is about 3 mol % of the lipid component. In certain embodiments, the amount of the ionizable lipid is about 32.9 mol % of the lipid component; the amount of the neutral lipid is about 15.2 mol % of the lipid component; the amount of the helper lipid is about 49.2 mol % of the lipid component; and the amount of the PEG lipid is about 2.7 mol % of the lipid component.

In certain embodiments, the amount of the ionizable lipid (e.g., Lipid A or one of its analogs) is about 20-50 mol %, about 25-34 mol %, about 25-38 mol %, about 25-45 mol %, about 29-38 mol %, about 29-43 mol %, about 29-34 mol %, about 30-34 mol %, about 30-38 mol %, about 30-43 mol %, about 30-43 mol %, or about 33 mol %. In certain embodiments, the amount of the neutral lipid is about 10-30 mol %, about 11-30 mol %, about 11-20 mol %, about 13-17 mol %, or about 15 mol %. In certain embodiments, the amount of the helper lipid is about 35-50 mol %, about 35-65 mol %, about 35-55 mol %, about 38-50 mol %, about 38-55 mol %, about 38-65 mol %, about 40-50 mol %, about 40-65 mol %, about 43-65 mol %, about 43-55 mol %, or about 49 mol %. In certain embodiments, the amount of the PEG lipid is about 1.5-3.5 mol %, about 2.0-2.7 mol %, about 2.0-3.5 mol %, about 2.3-3.5 mol %, about 2.3-2.7 mol %, about 2.5-3.5 mol %, about 2.5-2.7 mol %, about 2.9-3.5 mol %, or about 2.7 mol %.

Other embodiments of the present disclosure provide LNP compositions, for example, LNP compositions comprising an ionizable lipid (e.g., Lipid D or one of its analogs), a helper lipid, a helper lipid, and a PEG lipid, described according to the respective molar ratios of the component lipids in the formulation. In certain embodiments, the amount of the ionizable lipid is from about 25 mol % to about 50 mol %; the amount of the neutral lipid is from about 7 mol % to about 25 mol %; the amount of the helper lipid is from about 39 mol % to about 65 mol %; and the amount of the PEG lipid is from about 0.5 mol % to about 1.8 mol %. In certain embodiments, the amount of the ionizable lipid is from about 27-40 mol % of the lipid component; the amount of the neutral lipid is from about 10-20 mol % of the lipid component; the amount of the helper lipid is from about 50-60 mol % of the lipid component; and the amount of the PEG lipid is from about 0.9-1.6 mol % of the lipid component. In certain embodiments, the amount of the ionizable lipid is from about 30-45 mol % of the lipid component; the amount of the neutral lipid is from about 10-15 mol % of the lipid component; the amount of the helper lipid is from about 39-59 mol % of the lipid component; and the amount of the PEG lipid is from about 1-1.5 mol % of the lipid component. In certain embodiments, the amount of the ionizable lipid is from about 30-45 mol % of the lipid component; the amount of the neutral lipid is from about 10-15 mol % of the lipid component; the amount of the helper lipid is from about 39-59 mol % of the lipid component; and the amount of the PEG lipid is from about 1-1.5 mol % of the lipid component. In certain embodiments, the ionizable lipid is about 30 mol % of the lipid component; the amount of the neutral lipid is about 10 mol % of the lipid component; the amount of the helper lipid is about 59 mol % of the lipid component; and the amount of the PEG lipid is about 1-1.5 mol % of the lipid component. In certain embodiments, the amount of the ionizable lipid is about 40 mol % of the lipid component; the amount of the neutral lipid is about 15 mol % of the lipid component; the amount of the helper lipid is about 43.5 mol % of the lipid component; and the amount of the PEG lipid is about 1.5 mol % of the lipid component. In certain embodiments, the amount of the ionizable lipid is about 50 mol % of the lipid component; the amount of the neutral lipid is about 10 mol % of the lipid component; the amount of the helper lipid is about 39 mol % of the lipid component; and the amount of the PEG lipid is about 1 mol % of the lipid component.

In certain embodiments, the amount of the ionizable lipid (e.g., Lipid D or one of its analogs) is about 20-55 mol %, about 20-45 mol %, about 20-40 mol %, about 27-40 mol %, about 27-45 mol %, about 27-55 mol %, about 30-40 mol %, about 30-45 mol %, about 30-55 mol %, about 30 mol %, about 40 mol %, or about 50 mol %. In certain embodiments, the amount of the neutral lipid is about 7-25 mol %, about 10-25 mol %, about 10-20 mol %, about 15-20 mol %, about 8-15 mol %, about 10-15 mol %, about 10 mol %, or about 15 mol %. In certain embodiments, the amount of the helper lipid is about 39-65 mol %, about 39-59 mol %, about 40-60 mol %, about 40-65 mol %, about 40-59 mol %, about 43-65 mol %, about 43-60 mol %, about 43-59 mol %, or about 50-65 mol %, about 50-59 mol %, about 59 mol %, or about 43.5 mol %. In certain embodiments, the amount of the PEG lipid is about 0.5-1.8 mol %, about 0.8-1.6 mol %, about 0.8-1.5 mol %, 0.9-1.8 mol %, about 0.9-1.6 mol %, about 0.9-1.5 mol %, 1-1.8 mol %, about 1-1.6 mol %, about 1-1.5 mol %, about 1 mol %, or about 1.5 mol %.

In some embodiments, the cargo includes an mRNA encoding an RNA-guided DNA-binding agent (e.g. a Cas nuclease, a Class 2 Cas nuclease, or Cas9), or a gRNA or a nucleic acid encoding a gRNA, or a combination of mRNA and gRNA. In one embodiment, a LNP may comprise a Lipid A or its equivalents, or an amine lipid as provided in WO2020219876; or Lipid D or an amine lipid provided in WO2020/072605. In some aspects, the amine lipid is Lipid A, or Lipid D. In some aspects, the amine lipid is a Lipid A equivalent, e.g. an analog of Lipid A, or an amine lipid provided in WO2020/219876. In certain aspects, the amine lipid is an acetal analog of Lipid A, optionally, an amine lipid provided in WO2020/219876. In some aspects, the amine lipid is a Lipid D or an amine lipid found in in W2020072605. In various embodiments, a LNP comprises an amine lipid, a neutral lipid, a helper lipid, and a PEG lipid. In some embodiments, the helper lipid is cholesterol. In some embodiments, the neutral lipid is DSPC. In specific embodiments, PEG lipid is PEG2k-DMG. In some embodiments, a LNP may comprise a Lipid A, a helper lipid, a neutral lipid, and a PEG lipid. In some embodiments, a LNP comprises an amine lipid, DSPC, cholesterol, and a PEG lipid. In some embodiments, the LNP comprises a PEG lipid comprising DMG. In some embodiments, the amine lipid is selected from Lipid A, and an equivalent of Lipid A, including an acetal analog of Lipid A, or an amine lipid provided in WO2020/219876; or Lipid D or an amine lipid provided in WO2020/072605. In additional embodiments, a LNP comprises Lipid A, cholesterol, DSPC, and PEG2k-DMG. In additional embodiments, a LNP comprises Lipid D, cholesterol, DSPC, and PEG2k-DMG.

Embodiments of the present disclosure also provide lipid compositions described according to the molar ratio between the positively charged amine groups of the amine lipid (N) and the negatively charged phosphate groups (P) of the nucleic acid to be encapsulated. This may be mathematically represented by the equation N/P. In some embodiments, a LNP may comprise a lipid component that comprises an amine lipid, a helper lipid, a neutral lipid, and a helper lipid; and a nucleic acid component, wherein the N/P ratio is about 3 to 10. In some embodiments, the LNPs comprise molar ratios of an amine lipid to RNA/DNA phosphate (N:P) of about 4.5, 5.0, 5.5, 6.0, or 6.5. In some embodiments, a LNP may comprise a lipid component that comprises an amine lipid, a helper lipid, a neutral lipid, and a helper lipid; and an RNA component, wherein the N/P ratio is about 3 to 10. In one embodiment, the N/P ratio may about 5-7. In one embodiment, the N/P ratio may about 4.5-8. In one embodiment, the N/P ratio may about 6. In one embodiment, the N/P ratio may be 6±1. In one embodiment, the N/P ratio may about 6±0.5. In some embodiments, the N/P ratio will be ±30%, 25%, ±20%, 15%, ±10%, 5%, or ±2.5% of the target N/P ratio. In some embodiments, lipid nucleic acid assembly inter-lot variability will be less than 15%, less than 10% or less than 5%.

In some embodiments, the lipid nucleic acid assembly comprises an RNA component, which may comprise an mRNA, such as an mRNA encoding a Cas nuclease. In one embodiment, RNA component may comprise a Cas9 mRNA. In some compositions comprising an mRNA encoding a Cas nuclease, the lipid nucleic acid assembly further comprises a gRNA nucleic acid, such as a gRNA. In some embodiments, the RNA component comprises a Cas nuclease mRNA and a gRNA. In some embodiments, the RNA component comprises a Class 2 Cas nuclease mRNA and a gRNA.

In some embodiments, a LNP may comprise an mRNA encoding a Cas nuclease such as a Class 2 Cas nuclease, an amine lipid, a helper lipid, a neutral lipid, and a PEG lipid. In certain LNPs comprising an mRNA encoding a Cas nuclease such as a Class 2 Cas nuclease, the helper lipid is cholesterol. In other compositions comprising an mRNA encoding a Cas nuclease such as a Class 2 Cas nuclease, the neutral lipid is DSPC. In additional embodiments comprising an mRNA encoding a Cas nuclease such as a Class 2 Cas nuclease, the PEG lipid is PEG2k-DMG or PEG2k-C11. In specific compositions comprising an mRNA encoding a Cas nuclease such as a Class 2 Cas nuclease, the amine lipid is selected from Lipid A and its equivalents, such as an acetal analog of Lipid A, or amine lipids provided in WO2020/219876; or Lipid D and amine lipids provided in WO2020/072605.

In some embodiments, a LNP may comprise a gRNA. In some embodiments, a LNP may comprise an amine lipid, a gRNA, a helper lipid, a neutral lipid, and a PEG lipid. In certain LNPs comprising a gRNA, the helper lipid is cholesterol. In some compositions comprising a gRNA, the neutral lipid is DSPC. In additional embodiments comprising a gRNA, the PEG lipid is PEG2k-DMG or PEG2k-C11. In some embodiments, the amine lipid is selected from Lipid A and its equivalents, such as an acetal analog of Lipid A, or amine lipids provided in WO2020/219876 and their equivalents; or Lipid D and amine lipids provided in WO2020/072605 and their equivalents.

In one embodiment, a LNP may comprise an sgRNA. In one embodiment, a LNP may comprise a Cas9 sgRNA. In one embodiment, a LNP may comprise a Cpf1 sgRNA. In some compositions comprising an sgRNA, the lipid nucleic acid assembly includes an amine lipid, a helper lipid, a neutral lipid, and a PEG lipid. In certain compositions comprising an sgRNA, the helper lipid is cholesterol. In other compositions comprising an sgRNA, the neutral lipid is DSPC. In additional embodiments comprising an sgRNA, the PEG lipid is PEG2k-DMG or PEG2k-C11. In some embodiments, the amine lipid is selected from Lipid A and its equivalents, such as acetal analogs of Lipid A, or amine lipids provided in WO2020/219876; or Lipid D and amine lipids provided in WO2020/072605.

In some embodiments, a LNP comprises an mRNA encoding a Cas nuclease and a gRNA, which may be an sgRNA. In one embodiment, a LNP may comprise an amine lipid, an mRNA encoding a Cas nuclease, a gRNA, a helper lipid, a neutral lipid, and a PEG lipid. In certain compositions comprising an mRNA encoding a Cas nuclease and a gRNA, the helper lipid is cholesterol. In some compositions comprising an mRNA encoding a Cas nuclease and a gRNA, the neutral lipid is DSPC. In additional embodiments comprising an mRNA encoding a Cas nuclease and a gRNA, the PEG lipid is PEG2k-DMG or PEG2k-C11. In some embodiments, the amine lipid is selected from Lipid A and its equivalents, such as acetal analogs of Lipid A, or amine lipids provided in WO2020/219876; or Lipid D and amine lipids provided in WO2020/072605.

In some embodiments, the LNPs include a Cas nuclease mRNA, such as a Class 2 Cas mRNA and at least one gRNA. In some embodiments, the LNP includes a ratio of gRNA to Cas nuclease mRNA, such as Class 2 Cas nuclease mRNA from about 25:1 to about 1:25 wt/wt. In some embodiments, the lipid nucleic acid assembly formulation includes a ratio of gRNA to Cas nuclease mRNA, such as Class 2 Cas nuclease mRNA from about 10:1 to about 1:10. In some embodiments, the lipid nucleic acid assembly formulation includes a ratio of gRNA to Cas nuclease mRNA, such as Class 2 Cas nuclease mRNA from about 8:1 to about 1:8. As measured herein, the ratios are by weight. In some embodiments, the lipid nucleic acid assembly formulation includes a ratio of gRNA to Cas nuclease mRNA, such as Class 2 Cas mRNA from about 5:1 to about 1:5. In some embodiments, ratio range is about 3:1 to 1:3, about 2:1 to 1:2, about 5:1 to 1:2, about 5:1 to 1:1, about 3:1 to 1:2, about 3:1 to 1:1, about 3:1, about 2:1 to 1:1. In some embodiments, the gRNA to mRNA ratio is about 3:1 or about 2:1. In some embodiments the ratio of gRNA to Cas nuclease mRNA, such as Class 2 Cas nuclease is about 1:1. In some embodiments the ratio of gRNA to Cas nuclease mRNA, such as Class 2 Cas nuclease is about 1:2. The ratio may be about 25:1, 10:1, 5:1, 3:1, 2:1, 1:1, 1:2, 1:3, 1:5, 1:10, or 1:25.

The LNPs disclosed herein may include a template nucleic acid. The template nucleic acid may be co-formulated with an mRNA encoding a Cas nuclease, such as a Class 2 Cas nuclease mRNA. In some embodiments, the template nucleic acid may be co-formulated with a guide RNA. In some embodiments, the template nucleic acid may be co-formulated with both an mRNA encoding a Cas nuclease and a guide RNA. In some embodiments, the template nucleic acid may be formulated separately from an mRNA encoding a Cas nuclease or a guide RNA. The template nucleic acid may be delivered with, or separately from the LNPs. In some embodiments, the template nucleic acid may be single- or double-stranded, depending on the desired repair mechanism. The template may have regions of homology to the target DNA, or to sequences adjacent to the target DNA.

In some embodiments, lipid nucleic acid assemblies are formed by mixing an aqueous RNA solution with an organic solvent-based lipid solution, e.g., 100% ethanol. Suitable solutions or solvents include or may contain: water, PBS, Tris buffer, NaCl, citrate buffer, ethanol, chloroform, diethylether, cyclohexane, tetrahydrofuran, methanol, isopropanol. A pharmaceutically acceptable buffer, e.g., for in vivo administration of lipid nucleic acid assemblies, may be used. In some embodiments, a buffer is used to maintain the pH of the composition comprising lipid nucleic acid assemblies at or above pH 6.5. In some embodiments, a buffer is used to maintain the pH of the composition comprising lipid nucleic acid assemblies at or above pH 7.0. In some embodiments, the composition has a pH ranging from about 7.2 to about 7.7. In additional embodiments, the composition has a pH ranging from about 7.3 to about 7.7 or ranging from about 7.4 to about 7.6. In further embodiments, the composition has a pH of about 7.2, 7.3, 7.4, 7.5, 7.6, or 7.7. The pH of a composition may be measured with a micro pH probe. In some embodiments, a cryoprotectant is included in the composition. Non-limiting examples of cryoprotectants include sucrose, trehalose, glycerol, DMSO, and ethylene glycol. Exemplary compositions may include up to 10% cryoprotectant, such as, for example, sucrose. In some embodiments, the LNP may include about 1, 2, 3, 4, 5, 6, 7, 8, 9, or 10% cryoprotectant. In some embodiments, the LNP may include about 1, 2, 3, 4, 5, 6, 7, 8, 9, or 10% sucrose. In some embodiments, the LNP may include a buffer. In some embodiments, the buffer may comprise a phosphate buffer (PBS), a Tris buffer, a citrate buffer, and mixtures thereof. In some exemplary embodiments, the buffer comprises NaCl. In some embodiments, NaCl is omitted. Exemplary amounts of NaCl may range from about 20 mM to about 45 mM. Exemplary amounts of NaCl may range from about 40 mM to about 50 mM. In some embodiments, the amount of NaCl is about 45 mM. In some embodiments, the buffer is a Tris buffer. Exemplary amounts of Tris may range from about 20 mM to about 60 mM. Exemplary amounts of Tris may range from about 40 mM to about 60 mM. In some embodiments, the amount of Tris is about 50 mM. In some embodiments, the buffer comprises NaCl and Tris. Certain exemplary embodiments of the LNPs contain 5% sucrose and 45 mM NaCl in Tris buffer. In other exemplary embodiments, compositions contain sucrose in an amount of about 5% w/v, about 45 mM NaCl, and about 50 mM Tris at pH 7.5. The salt, buffer, and cryoprotectant amounts may be varied such that the osmolality of the overall formulation is maintained. For example, the final osmolality may be maintained at less than 450 mOsm/L. In further embodiments, the osmolality is between 350 and 250 mOsm/L. Certain embodiments have a final osmolality of 300+/−20 mOsm/L.

In some embodiments, microfluidic mixing, T-mixing, or cross-mixing is used. In certain aspects, flow rates, junction size, junction geometry, junction shape, tube diameter, solutions, or RNA and lipid concentrations may be varied. Lipid nucleic acid assemblies or LNPs may be concentrated or purified, e.g., via dialysis, tangential flow filtration, or chromatography. The lipid nucleic acid assemblies may be stored as a suspension, an emulsion, or a lyophilized powder, for example. In some embodiments, a LNP is stored at 2-8° C., in certain aspects, the LNPs are stored at room temperature. In additional embodiments, a LNP is stored frozen, for example at −20° C. or −80° C. In other embodiments, a LNP is stored at a temperature ranging from about 0° C. to about −80° C. Frozen LNPs may be thawed before use, for example on ice, at 4° C., at room temperature, or at 25° C. Frozen LNPs may be maintained at various temperatures, for example on ice, at 4° C., at room temperature, at 25° C., or at 37° C.

In some embodiments, the concentration of the LNPs in the LNP composition is about 1-10 ug/mL, about 2-10 ug/mL, about 2.5-10 ug/mL, about 1-5 ug/mL, about 2-5 ug/mL, about 2.5-5 ug/mL, about 0.04 ug/mL, about 0.08 ug/mL, about 0.16 ug/mL, about 0.25 ug/mL, about 0.63 ug/mL, about 1.25 ug/mL, about 2.5 ug/mL, or about 5 ug/mL.

In some embodiments, the LNP comprises a stealth lipid, optionally wherein:
(i) the LNP comprises a lipid component and the lipid component comprises: about 50-60 mol % amine lipid such as Lipid A or Lipid D, about 8-10 mol % neutral lipid; and about 2.5-4 mol % stealth lipid (e.g., a PEG lipid), wherein the remainder of the lipid component is helper lipid, and wherein the N/P ratio of the LNP is about 6;
(ii) the LNP comprises about 50-60 mol % amine lipid such as Lipid A or Lipid D; about 27-39.5 mol % helper lipid; about 8-10 mol % neutral lipid; and about 2.5-4 mol % stealth lipid (e.g., a PEG lipid), wherein the N/P ratio of the LNP is about 5-7 (e.g., about 6);
(iii) the LNP comprises a lipid component and the lipid component comprises: about 50-60 mol % amine lipid such as Lipid A or Lipid D; about 5-15 mol % neutral lipid; and about 2.5-4 mol % stealth lipid (e.g., a PEG lipid), wherein the remainder of the lipid component is helper lipid, and wherein the N/P ratio of the LNP is about 3-10;
(iv) the LNP comprises a lipid component and the lipid component comprises: about 40-60 mol % amine lipid such as Lipid A or Lipid D; about 5-15 mol % neutral lipid; and about 2.5-4 mol % stealth lipid (e.g., a PEG lipid), wherein the remainder of the lipid component is helper lipid, and wherein the N/P ratio of the LNP is about 6;
(v) the LNP comprises a lipid component and the lipid component comprises: about 50-60 mol % amine lipid such as Lipid A or Lipid D; about 5-15 mol % neutral lipid; and about 1.5-10 mol % stealth lipid (e.g., a PEG lipid), wherein the remainder of the lipid component is helper lipid, and wherein the N/P ratio of the LNP is about 6;
(vi) the LNP comprises a lipid component and the lipid component comprises: about 40-60 mol % amine lipid such as Lipid A or Lipid D; about 0-10 mol % neutral lipid; and about 1.5-10 mol % stealth lipid (e.g., a PEG lipid), wherein the remainder of the lipid component is helper lipid, and wherein the N/P ratio of the LNP is about 3-10;
(vii) the LNP comprises a lipid component and the lipid component comprises: about 40-60 mol % amine lipid such as Lipid A or Lipid D; less than about 1 mol % neutral lipid; and about 1.5-10 mol % stealth lipid (e.g., a PEG lipid), wherein the remainder of the lipid component is helper lipid, and wherein the N/P ratio of the LNP is about 3-10;
(viii) the LNP comprises a lipid component and the lipid component comprises: about 40-60 mol % amine lipid such as Lipid A or Lipid D; and about 1.5-10 mol % stealth lipid (e.g., a PEG lipid), wherein the remainder of the lipid component is helper lipid, wherein the N/P ratio of the LNP composition is about 3-10, and wherein the LNP is essentially free of or free of neutral phospholipid; or
(ix) the LNP comprises a lipid component and the lipid component comprises: about 50-60 mol % amine lipid such as Lipid A or Lipid D; about 8-10 mol-% neutral lipid; and about 2.5-4 mol % stealth lipid (e.g., a PEG lipid), wherein the remainder of the lipid component is helper lipid, and wherein the N/P ratio of the LNP is about 3-7.

In some embodiments, the LNP comprises a lipid component and the lipid component comprises: about 50 mol % amine lipid such as Lipid A or Lipid D; about 9 mol % neutral lipid such as DSPC; about 3 mol % of stealth lipid such as a PEG lipid, such as PEG2k-DMG, and the remainder of the lipid component is helper lipid such as cholesterol wherein the N/P ratio of the LNP is about 6.

In some embodiments, the LNP comprises a lipid component and the lipid component comprises: about 50 mol % Lipid A; about 9 mol % DSPC; about 3 mol % of PEG2k-DMG, and the remainder of the lipid component is cholesterol wherein the N/P ratio of the LNP is about 6.

In some embodiments, the LNP comprises a lipid component and the lipid component comprises: about 35 mol % Lipid A; about 15 mol % neutral lipid; about 47.5 mol % helper lipid; and about 2.5 mol % stealth lipid (e.g., PEG lipid), and wherein the N/P ratio of the LNP composition is about 3-7.

In some embodiments, the LNP comprises a lipid component and the lipid component comprises: about 35 mol % Lipid D; about 15 mol % neutral lipid; about 47.5 mol % helper lipid; and about 2.5 mol % stealth lipid (e.g., PEG lipid), and wherein the N/P ratio of the LNP composition is about 3-7.

In some embodiments, the LNP comprises a lipid component and the lipid component comprises: about 25-45 mol % amine lipid, such as Lipid A; about 10-30 mol % neutral lipid; about 25-65 mol % helper lipid; and about 1.5-3.5 mol % stealth lipid (e.g., PEG lipid), and wherein the N/P ratio of the LNP composition is about 3-7.

In some embodiments, the LNP comprises a lipid component, wherein:
a. the amount of the amine lipid is about 29-44 mol % of the lipid component; the amount of the neutral lipid is about 11-28 mol % of the lipid component; the amount of the helper lipid is about 28-55 mol % of the lipid component; and the amount of the PEG lipid is about 2.3-3.5 mol % of the lipid component
b. the amount of the amine lipid is about 29-38 mol % of the lipid component; the amount of the neutral lipid is about 11-20 mol % of the lipid component; the amount of the helper lipid is about 43-55 mol % of the lipid component; and the amount of the PEG lipid is about 2.3-2.7 mol % of the lipid component;
c. the amount of the amine lipid is about 25-34 mol % of the lipid component; the amount of the neutral lipid is about 10-20 mol % of the lipid component; the amount of the helper lipid is about 45-65 mol % of the lipid component; and the amount of the PEG lipid is about 2.5-3.5 mol % of the lipid component; or
d. the amount of the amine lipid is about 30-43 mol % of the lipid component; the amount of the neutral lipid is about 10-17 mol % of the lipid component; the amount of the helper lipid is about 43.5-56 mol % of the lipid component; and the amount of the PEG lipid is about 1.5-3 mol % of the lipid component.

In some embodiments, the LNP comprises a lipid component and the lipid component comprises: about 25-50 mol % amine lipid, such as Lipid D; about 7-25 mol % neutral lipid; about 39-65 mol % helper lipid; and about 0.5-1.8 mol % stealth lipid (e.g., PEG lipid), and wherein the N/P ratio of the LNP composition is about 3-7.

In some embodiments, the LNP comprises a lipid component wherein the amount of the amine lipid is about 30-45 mol % of the lipid component; or about 30-40 mol % of the lipid component; optionally about 30 mol %, 40 mol %, or 50 mol % of the lipid component. In some embodiments, the LNP comprises a lipid component wherein the amount of the neutral lipid is about 10-20 mol % of the lipid component; or about 10-15 mol % of the lipid component; optionally about 10 mol % or 15 mol % of the lipid component. In some embodiments, the LNP comprises a lipid component wherein the amount of the helper lipid is about 50-60 mol % of the lipid component; about 39-59 mol % of the lipid component; or about 43.5-59 mol % of the lipid component; optionally about 59 mol % of the lipid component; about 43.5 mol % of the lipid component; or about 39 mol % of the lipid component. In some embodiments, the LNP comprises a lipid component wherein the amount of the PEG lipid is about 0.9-1.6 mol % of the lipid component; or about 1-1.5 mol % of the lipid component; optionally about 1 mol % of the lipid component or about 1.5 mol % of the lipid component

In some embodiments, the LNP comprises a lipid component, wherein:
a. the amount of the ionizable lipid is about 27-40 mol % of the lipid component; the amount of the neutral lipid is about 10-20 mol % of the lipid component; the amount of the helper lipid is about 50-60 mol % of the lipid component; and the amount of the PEG lipid is about 0.9-1.6 mol % of the lipid component;
b. the amount of the ionizable lipid is from about 30-45 mol % of the lipid component; the amount of the neutral lipid is from about 10-15 mol % of the lipid component; the amount of the helper lipid is from about 39-59 mol % of the lipid component; and the amount of the PEG lipid is from about 1-1.5 mol % of the lipid component;
c. the amount of the ionizable lipid is about 30 mol % of the lipid component; the amount of the neutral lipid is about 10 mol % of the lipid component; the amount of the helper lipid is about 59 mol % of the lipid component; and the amount of the PEG lipid is about 1-1.5 mol % of the lipid component;
d. the amount of the ionizable lipid is about 40 mol % of the lipid component; the amount of the neutral lipid is about 15 mol % of the lipid component; the amount of the helper lipid is about 43.5 mol % of the lipid component; and the amount of the PEG lipid is about 1.5 mol % of the lipid component; or
e. the amount of the ionizable lipid is about 50 mol % of the lipid component; the amount of the neutral lipid is about 10 mol % of the lipid component; the amount of the helper lipid is about 39 mol % of the lipid component; and the amount of the PEG lipid is about 1 mol % of the lipid component.

In some embodiments, the LNP has a diameter of about 1-250 nm, 10-200 nm, about 20-150 nm, about 50-150 nm, about 50-100 nm, about 50-120 nm, about 60-100 nm, about 75-150 nm, about 75-120 nm, or about 75-100 nm. In some embodiments, the LNP has a diameter of less than 100 nm. In some embodiments, the LNP composition comprises a population of the LNP with an average diameter of about 10-200 nm, about 20-150 nm, about 50-150 nm, about 50-100 nm, about 50-120 nm, about 60-100 nm, about 75-150 nm, about 75-120 nm, or about 75-100 nm. In some embodiments, the LNP has an average diameter of less than 100 nm.

In some embodiments, the LNP comprises: about 40-60 mol-% amine lipid; about 5-15 mol-% neutral lipid; and about 1.5-10 mol-% PEG lipid, wherein the remainder of the lipid component is helper lipid, and wherein the N/P ratio of the LNP composition is about 3-10. In some embodiments, the LNP comprises: about 50-60 mol-% amine lipid; about 8-10 mol-% neutral lipid; and about 2.5-4 mol-% PEG lipid, wherein the remainder of the lipid component is helper lipid, and wherein the N/P ratio of the LNP composition is about 3-8. In some embodiments, the LNP comprises: about 50-60 mol-% amine lipid; about 5-15 mol-% DSPC; and about 2.5-4 mol-% PEG lipid, wherein the remainder of the lipid component is cholesterol, and wherein the N/P ratio of the LNP composition is 3-8±0.2.

In embodiments, the average diameter is a Z-average diameter. In certain embodiments, the Z-average diameter is measured by dynamic light scattering (DLS) using methods known in the art. For example, average particle size and polydispersity can be measured by dynamic light scattering (DLS) using a Malvern Zetasizer DLS instrument. LNP samples are diluted with PBS buffer prior to being measured by DLS. Z-average diameter and number average diameter along with a polydispersity index (pdi) can be determined. The Z average is the intensity weighted mean hydrodynamic size of the ensemble collection of particles. The number average is the particle number weighted mean hydrodynamic size of the ensemble collection of particles. A Malvern Zetasizer instrument can also be used to measure the zeta potential of the LNP using methods known in the art.

### D. Arrangement of Components in LNPs

In some embodiments, the first genome editing tool, the second genome editing tool, or the at least one gRNA is contained in at least one LNP. In some embodiments, the first genome editing tool, the second genome editing tool, and the gRNAs are collectively contained in: (i) a first lipid nanoparticle (LNP) comprising the second genomic editor and a first gRNA, (ii) a second LNP comprising the first genomic editor or the base editor, (iii) a third LNP comprising a uracil glycosylase inhibitor (UGI), (iv) a fourth LNP comprising a second gRNA, (v) a fifth LNP comprising a third gRNA, and (vi) a sixth LNP comprising a fourth gRNA. In some embodiments, the first genome editing tool, the second genome editing tool, and the gRNAs are collectively contained in: (i) a first lipid nanoparticle (LNP) comprising the second genomic editor and a first gRNA, (ii) a second LNP comprising the first genomic editor or the base editor, (iii) a third LNP comprising a uracil glycosylase inhibitor (UGI), (iv) a fourth LNP comprising a second gRNA and a third gRNA, and (v) a fifth LNP comprising a fourth gRNA. In some embodiments, the first genome editing tool, the second genome editing tool, and the gRNAs are collectively contained in: (i) a first lipid nanoparticle (LNP) comprising the second genomic editor and a first gRNA, (ii) a second LNP comprising the first genomic editor or the base editor and comprising a uracil glycosylase inhibitor (UGI), (iii) a third LNP comprising a second gRNA, (iv) a fourth LNP comprising a third gRNA, and (v) a fifth LNP comprising a fourth gRNA. In some embodiments, the first genome editing tool, the second genome editing tool, and the gRNAs are collectively contained in: (i) a first lipid nanoparticle (LNP) comprising the second genomic editor and a first gRNA, (ii) a second LNP comprising the first genomic editor or the base editor and comprising a uracil glycosylase inhibitor (UGI), (iii) a third LNP comprising a second gRNA and a third gRNA, and (iv) a fourth LNP comprising a fourth gRNA. In some embodiments, the first genome editing tool, the second genome editing tool, and the gRNAs are collectively contained in: (i) a first lipid nanoparticle (LNP) comprising the second genomic editor and a first gRNA, (ii) a second LNP comprising the first genomic editor or the base editor, (iii) a third LNP comprising a uracil glycosylase inhibitor (UGI), (iv) a fourth LNP comprising a second gRNA, a third gRNA, and a fourth gRNA. In some embodiments, the first genome editing tool, the second genome editing tool, and the gRNAs are collectively contained in: (i) a first lipid nanoparticle (LNP) comprising the second genomic editor and a first gRNA, (ii) a second LNP comprising a uracil glycosylase inhibitor (UGI), (iii) a third LNP comprising the first genomic editor or the base editor and comprising a second gRNA, (iv) a fourth LNP comprising the first genomic editor or the base editor and comprising a third gRNA, and (v) a fifth LNP comprising the first genomic editor or the base editor and comprising a fourth gRNA. In some embodiments, the first genome editing tool, the second genome editing tool, and the gRNAs are collectively contained in: (i) a first lipid nanoparticle (LNP) comprising the second genomic editor and a first gRNA, (ii) a second LNP comprising a uracil glycosylase inhibitor (UGI), (iii) a third LNP comprising the first genomic editor or the base editor and comprising a second gRNA and a third gRNA, and (iv) a fourth LNP comprising the first genomic editor or the base editor and comprising a fourth gRNA. In some embodiments, the first genome editing tool, the second genome editing tool, and the gRNAs are collectively contained in the first through fourth LNPs, the first through fifth LNPs, or the first through sixth LNPs, and in one or more additional LNP comprising a fifth gRNA. In some embodiments, the one or more additional LNP further comprises a sixth gRNA. In some embodiments, the one or more additional LNP further comprises a seventh gRNA. In some embodiments, the one or more additional LNP further comprises an eighth gRNA. In some embodiments, the one or more additional LNP further comprises a ninth gRNA. In some embodiments, the one or more additional LNP further comprises a tenth gRNA.

In some embodiments, the second genomic editor comprises an S. pyogenes (Spy) Cas9 cleavase, the first genomic editor or the base editor comprises an N. meningitidis (Nine) Cas9 nickase, the first gRNA targets the TRAC locus, the second gRNA targets the HLA-A locus, the third gRNA targets the CIITA locus, the fourth gRNA targets the HLA-B locus, the fifth gRNA targets the TRBC locus and the one or more additional gRNAs each targets a locus different from the TRAC locus, the HLA-A locus, the HLA-B locus, the CIITA locus, and the TRBC locus.

In some embodiments, the second genomic editor comprises an S. pyogenes (Spy) Cas9 cleavase, the first genomic editor or the base editor comprises an N. meningitidis (Nine) Cas9 nickase, the first gRNA targets the TRAC locus, the second gRNA targets the HLA-A locus, the third gRNA targets the CIITA locus, and the fourth gRNA targets the HLA-B locus, and the one or more additional gRNAs each targets a locus different from the TRAC locus, the HLA-A locus, the HLA-B locus, and the CIITA locus.

In some embodiments, the first gRNA comprises the sequence of SEQ ID NO: 374 or 378 or a sequence at least 95%, 90%, or 85% identical to SEQ ID NO: 374 or 378, wherein the second gRNA comprises the sequence of SEQ ID NO: 366 or 370 or a sequence at least 95%, 90%, or 85% identical to SEQ ID NO: 366 or 370, wherein the third gRNA comprises the sequence of SEQ ID NO: 345 or 384 or a sequence at least 95%, 90%, or 85% identical to SEQ ID NO: 345 or 384, and wherein the fourth gRNA comprises the sequence of SEQ ID NO: 363 or a sequence at least 95%, 90%, or 85% identical to SEQ ID NO: 363.

In some embodiments, the first genome editing tool, the second genome editing tool, and the gRNAs are collectively contained in at least 2, 3, 4, 5, 6, 7, 8, 9, or 10 distinct lipid nanoparticles (LNP) each comprising a distinct nucleic acid component. In some embodiments, the first genome editing tool, the second genome editing tool, and the gRNAs are collectively contained in 4, 5, 6, or 7 distinct lipid nanoparticles (LNP) each comprising a distinct nucleic acid component. In some embodiments, the first genome editing tool, the second genome editing tool, and the gRNAs are collectively contained in 4 distinct LNPs each comprising a distinct nucleic acid component. In some embodiments, the first genome editing tool, the second genome editing tool, and the gRNAs are collectively contained in 5 distinct LNPs each comprising a distinct nucleic acid component. In some embodiments, the first genome editing tool, the second genome editing tool, and the gRNAs are collectively contained in 6 distinct LNPs each comprising a distinct nucleic acid component. In some embodiments, the first genome editing tool, the second genome editing tool, and the gRNAs are collectively contained in 7 distinct LNPs each comprising a distinct nucleic acid component.

In some embodiments, the at least one gRNA that is cognate to the first genomic editor or the base editor and the at least one gRNA that is cognate to the second genomic editor collectively comprise at least 2 gRNAs, and wherein 2 of the gRNAs that target different genomic loci are contained in a same lipid nanoparticle (LNP). In some embodiments, the at least one gRNA that is cognate to the first genomic editor or the base editor and the at least one gRNA that is cognate to the second genomic editor collectively comprise at least 3 gRNAs, and wherein 3 of the gRNAs that target different genomic loci are contained in a same lipid nanoparticle. In some embodiments, the at least one gRNA that is cognate to the first genomic editor or the base editor and the at least one gRNA that is cognate to the second genomic editor collectively comprise at least 4 gRNAs, and wherein 4 of the gRNAs that target different genomic loci are contained in a same lipid nanoparticle.

In some embodiments, each of the other gRNAs is contained in a different LNP. In some embodiments, each one of the gRNAs is contained in a different LNP.

In some embodiments, the at least one gRNA that is cognate to the first genomic editor or the base editor comprises more than one gRNAs that target different genomic loci, and the first genomic editor or the base editor is contained in a same LNP with at least one of the more than one gRNAs. In some embodiments, the first genomic editor or the base editor and one of the gRNAs are contained in a same LNP. In some embodiments, the first genomic editor or the base editor and 2 of the gRNAs are contained in a same LNP.

In some embodiments, the first genomic editor or the base editor and 3 of the gRNAs are contained in a same LNP. In some embodiments, the first genomic editor or the base editor and 4 of the gRNAs are contained in a same LNP.

In some embodiments, the first genomic editor or the base editor is contained in a different LNP than each of the at least one gRNA that is cognate to the first genomic editor or the base editor.

In some embodiments, the at least one gRNA that is cognate to the first genomic editor or the base editor comprises more than one gRNAs that target different genomic loci, and each of the more than one gRNAs is contained in a different LNP.

In some embodiments, each of the LNPs comprising one of the gRNAs cognate to the first genomic editor or the base editor further comprises the first genomic editor or the base editor.

In some embodiments, the second genomic editor and the at least one gRNA that is cognate to the second genomic editor are contained in a same LNP. In some embodiments, the second genomic editor is contained in a same LNP with one of the gRNAs.

In some embodiments, the first genome editing tool comprises a uracil glycosylase inhibitor (UGI), and the UGI is contained in a different LNP than each one of the gRNAs.

In some embodiments, the LNPs comprise a first group of distinct LNPs, and a second group of distinct LNPs, and optionally, a third group of distinct LNPs. In some embodiments, the first group of distinct LNPs comprises 2, 3, 4, or 5 LNPs, the second group of distinct LNPs comprises 2, 3, 4, or 5 LNPs, and the third group of distinct LNPs, when present, comprises 2, 3, 4, or 5 LNPs. In some embodiments, the first group of distinct LNPs comprises 3 or 4 LNPs, the second group of distinct LNPs comprises 3 or 4 LNPs. In some embodiments, the first group of distinct LNPs, the second group of distinct LNPs, and the third group of distinct LNPs, when present, are delivered to the cell sequentially. In some embodiments, the second group of distinct LNPs is delivered to the cell 1, 2, or 3 days after the first group of distinct LNPs is delivered to the cell, and wherein the third group of distinct LNPs, when present, is delivered to the cell 1, 2, or 3 days after the second group of distinct LNPs is delivered to the cell.

In some embodiments, the first genome editing tool, the second genome editing tool, and the gRNAs are collectively contained in: (a) (i) a first lipid nanoparticle (LNP) comprising a uracil glycosylase inhibitor (UGI); (ii) a second LNP comprising the first genomic editor or the base editor and comprising a second gRNA; (iii) a third LNP comprising the first genomic editor or the base editor and comprising a third gRNA; and (iv) a fourth LNP comprising the first genomic editor or the base editor and comprising a fourth gRNA; and (b) (i) a fifth LNP comprising a uracil glycosylase inhibitor (UGI); (ii) a sixth LNP comprising the second genomic editor and a first gRNA; (iii) a nucleic acid encoding an exogenous gene for insertion at an editing site of the first gRNA; (iv) optionally a seventh LNP comprising the first genomic editor or the base editor and comprising a fifth gRNA; (v) optionally an eighth LNP comprising the first genomic editor or the base editor and comprising a sixth gRNA; (vi) optionally a ninth LNP comprising the first genomic editor or the base editor and comprising a seventh gRNA.

E. Contacting Cells with LNP

In some embodiments, the LNP is pretreated with a serum factor before contacting the cell. In some embodiments, the LNP is pretreated with a primate serum factor before contacting the cell. In some embodiments, the LNP is pretreated with a human serum factor before contacting the cell. In some embodiments, the LNP is pretreated with ApoE before contacting the cell. In some embodiments, the LNP is pretreated with a recombinant ApoE3 or ApoE4 before contacting the cell. In some embodiments, the cell is serum-starved prior to contact with the LNP.

In some embodiments, the multiplex methods comprise preincubating a serum factor and the LNP for about 30 seconds to overnight. In some embodiments, the preincubation step comprises preincubating a serum factor and the LNP for about 1 minute to 1 hour. In some embodiments, it comprises preincubating for about 1-30 minutes. In other embodiments, it comprises preincubating for about 1-10

In some embodiments, the LNP compositions are administered sequentially. In some embodiments, the LNP compositions are administered simultaneously. In some embodiments, the population of cells is contacted with 2-12 LNP compositions. In some embodiments, the population of cells is contacted with 2-8 LNP compositions. In some embodiments, the population of cells is contacted with 2-6 LNP compositions. In some embodiments, the population of cells is contacted with 3-8 LNP compositions. In some embodiments, the population of cells is contacted with 3-6 LNP compositions. In some embodiments, the population of cells is contacted with 4-6 LNP compositions. In some embodiments, the population of cells is contacted with 6-12 LNP compositions. In some embodiments, the population of cells is contacted with 3 LNP compositions. In some embodiments, the population of cells is contacted with 4 LNP compositions. In some embodiments, the population of cells is contacted with 6 LNP compositions. In some embodiments, the population of cells is contacted with 3 LNP compositions. In some embodiments, the population of cells is contacted with the LNP compositions simultaneously.

In some embodiments, the population of cells is contacted with no more than 6 LNP compositions simultaneously. In some embodiments, the population of cells is contacted with no more than 2 LNP compositions simultaneously.

In some embodiments, the cells are frozen between sequential contacting or editing steps.

In some embodiments, the LNP is pretreated with a serum factor before contacting the cell. In some embodiments, the LNP is pretreated with a human serum before contacting the cell. In some embodiments, the LNP is pretreated with a serum replacement, e.g., a commercially available serum replacement, preferably wherein the serum replacement is appropriate for ex vivo use. In some embodiments, the LNP is pretreated with ApoE before contacting the cell. In some embodiments, the LNP is pretreated with a recombinant ApoE3 or ApoE4 before contacting the cell. In some embodiments, the cell is serum-starved prior to contact with the LNP.

In some embodiments, the multiplex methods comprise preincubating a serum factor and the LNP for about 30 seconds to overnight. In some embodiments, the preincubation step comprises preincubating a serum factor and the LNP for about 1 minute to 1 hour. In some embodiments, it comprises preincubating for about 1-30 minutes. In other embodiments, it comprises preincubating for about 1-10 minutes. Still further embodiments comprise preincubating for about 5 minutes.

In some embodiments, the preincubating step occurs at about 4° C. In some embodiments, the preincubating step occurs at about 25° C. In some embodiments, the preincubating step occurs at about 37° C. The preincubating step may comprise a buffer such as sodium bicarbonate or HEPES.

In some embodiments, a LNP is provided to a “non-activated” cell. A “non-activated” cell refers to a cell that has not been stimulated in vitro. In some embodiments, a “non-activated” T cell may have been stimulated in vivo (e.g., by antigen) while in the body, however said cell may be referred to as non-activated herein if said cell has not been stimulated in vitro in culture. An “activated” cell is also useful in the methods disclosed herein and can refer to a cell that has been stimulated in vitro. Agents for activating cells in vitro are provided herein and are known in the art, particularly for activation of T cells or B cells.

In some embodiments, a T cell is cultured in culture medium prior to contact with a LNP. In some embodiments, the T cell is cultured with one or more proliferative cytokines, for example one or more or all of IL-2, IL-15, IL-7, and IL-21, or one or more agents that provides activation through CD3 or CD28.

In some embodiments, the T cell is activated prior to contact with a LNP, is activated in between contact with LNPs, or is activated after contact with a LNP.

In some embodiments, the cell is a T cell and the method further comprises an activation step between a first and a second contacting step. In some embodiments, a non-activated T cell is contacted with one, two, or three nucleic acid assembly compositions. In some embodiments, an activated T cell is contacted with one to 8 LNPs, optionally 1 to 4 LNPs. In some embodiments, the T cell is contacted with at least 6 LNPs. In some embodiments, the T cell is contacted with no more than 12 LNPs. In some embodiments, the T cell is contacted with 2-12 LNPs. In some embodiments, the T cell is contacted with 2-8 LNPs. In some embodiments, the T cell is contacted with 2-6 LNPs. In some embodiments, the T cell is contacted with 3-8 LNPs. In some embodiments, the T cell is contacted with 3-6 LNPs. In some embodiments, the T cell is contacted with 4-6 LNPs. In some embodiments, the T cell is contacted with 4-12 LNPs. In some embodiments, the T cell is contacted with 4-8 LNPs. In some embodiments, the T cell is contacted with 6-12 LNPs. In some embodiments, the T cell is contacted with 3, 4, 5, or 6 LNPs. In some embodiments, the T cell is contacted with no more than 8 LNPs simultaneously. In some embodiments, the T cell is contacted with no more than 6 LNPs simultaneously. In some embodiments, the activated T cell is contacted with at least 6 LNPs. In some embodiments, the activated T cell is contacted with no more than 12 LNPs. In some embodiments, the activated T cell is contacted with 2-12 LNPs. In some embodiments, the activated T cell is contacted with 4-12 LNPs. In some embodiments, the activated T cell is contacted with 4-8 LNPs. In some embodiments, the activated T cell is contacted with no more than 8 LNPs simultaneously. In some embodiments, the activated T cell is contacted with no more than 6 LNPs simultaneously.

### VIII. FURTHER EXEMPLARY EMBODIMENTS

While the invention is described in conjunction with the illustrated embodiments, it is understood that they are not intended to limit the invention to those embodiments. On the contrary, the invention is intended to cover all alternatives, modifications, and equivalents, including equivalents of specific features, which may be included within the invention as defined by the appended claims.

Both the foregoing general description and detailed description, as well as the following examples, are exemplary and explanatory only and are not restrictive of the teachings. The section headings used herein are for organizational purposes only and are not to be construed as limiting the desired subject matter in any way. In the event that any literature incorporated by reference contradicts any term defined in this specification, this specification controls. All ranges given in the application encompass the endpoints unless stated otherwise.

### IX. EXAMPLES

### Example 1. Materials and Methods

### Example 1.1. Next-Generation Sequencing (“NGS”) and Analysis for On-Target Cleavage Efficiency

Genomic DNA was extracted using QuickExtract™ DNA Extraction Solution (Lucigen, Cat. No. QE09050) according to manufacturer's protocol.

To quantitatively determine the efficiency of editing at the target location in the genome, deep sequencing was utilized to identify the presence of insertions, deletions, and substitution introduced by gene editing. PCR primers were designed around the target site within the gene of interest (e.g., HLA-A) and the genomic area of interest was amplified. Primer sequence design was done as is standard in the field.

Additional PCR was performed according to the manufacturer's protocols (Illumina) to add chemistry for sequencing. The amplicons were sequenced on an Illumina MiSeq instrument. The reads were aligned to the human reference genome (e.g., hg38) after eliminating those having low quality scores. Reads that overlapped the target region of interest were re-aligned to the local genome sequence to improve the alignment. Then the number of wild type reads versus the number of reads which contain C-to-T mutations, C-to-A/G mutations or indels was calculated. Insertions and deletions were scored in a 20 bp region centered on the predicted Cas9 cleavage site. Indel percentage is defined as the total number of sequencing reads with one or more base inserted or deleted within the 20 bp scoring region divided by the total number of sequencing reads, including wild type. C-to-T mutations or C-to-A/G mutations were scored in a 40 bp region including 10 bp upstream and 10 bp downstream of the 20 bp sgRNA target sequence. The C-to-T editing percentage is defined as the total number of sequencing reads with either one or more C-to-T mutations within the 40 bp region divided by the total number of sequencing reads, including wild type. The percentage of C-to-A/G mutations are calculated similarly.

### Example 1.2. Preparation of Lipid Nanoparticles

The lipid components were dissolved in 100% ethanol at various molar ratios. The RNA cargos (e.g., Cas9 mRNA and sgRNA) were dissolved in 25 mM citrate buffer, 100 mM NaCl, pH 5.0, resulting in a concentration of RNA cargo of approximately 0.45 mg/mL.

The lipid nucleic acid assemblies contained ionizable Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate), cholesterol, DSPC, and PEG2k-DMG in molar ratios listed in examples below. The lipid nucleic acid assemblies were formulated with a lipid amine to RNA phosphate (N:P) molar ratio of about 6, and a ratio of gRNA to mRNA of 1:1 or 1:2 by weight.

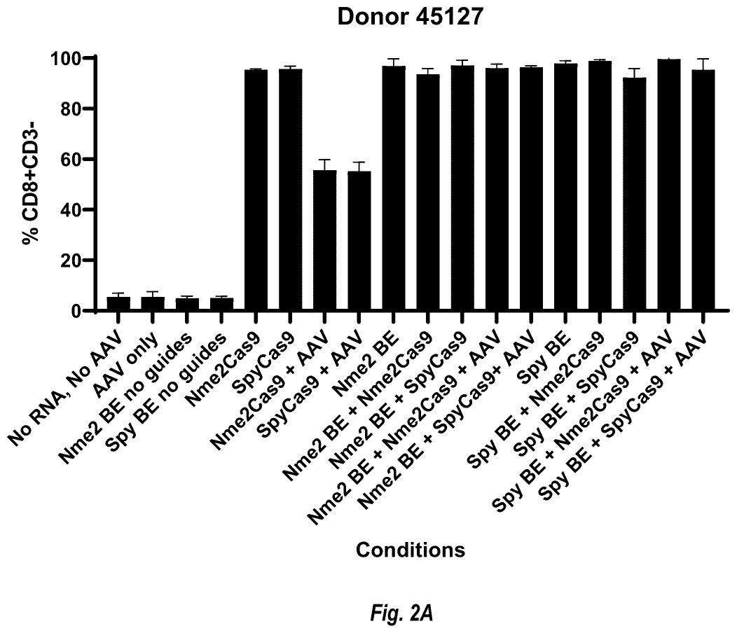

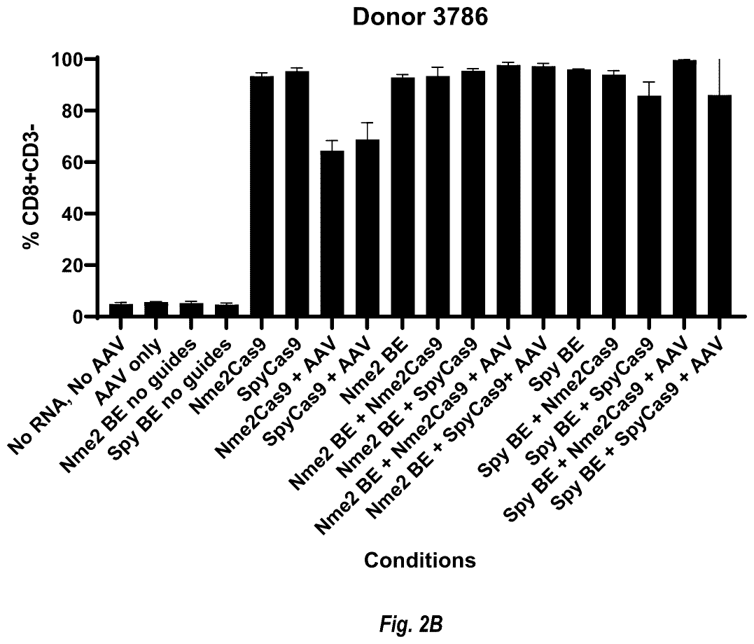

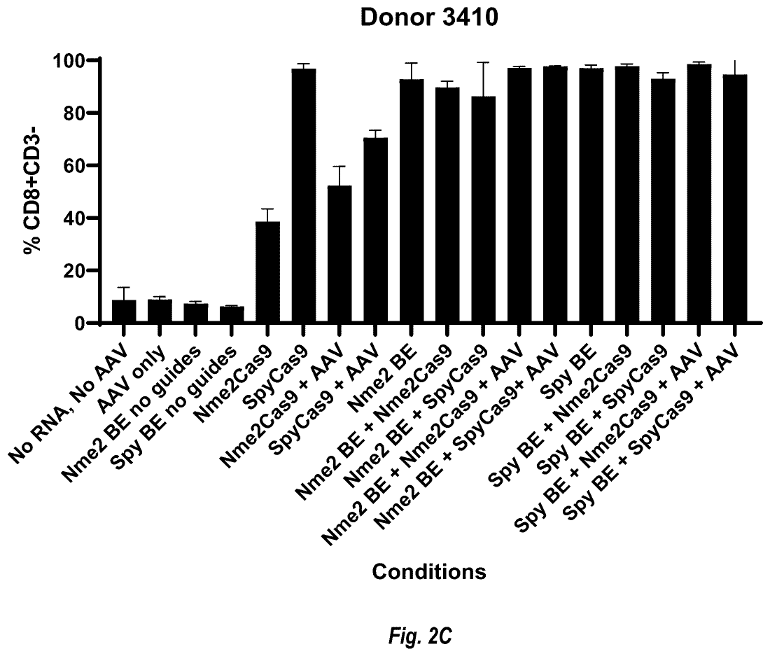

Lipid nanoparticles (LNP compositions) were prepared using a cross-flow technique utilizing impinging jet mixing of the lipid in ethanol with two volumes of RNA solutions and one volume of water. The lipids in ethanol were mixed through a mixing cross with the two volumes of RNA solution. A fourth stream of water was mixed with the outlet stream of the cross through an inline tee (See WO2016010840, FIG. 2). The LNP compositions were held for 1 hour at room temperature (RT), and further diluted with water (approximately 1:1 v/v). LNP compositions were concentrated using tangential flow filtration on a flat sheet cartridge (Sartorius, 100 kD MWCO) and buffer exchanged using PD-10 desalting columns (GE) into 50 mM Tris, 45 mM NaCl, 5% (w/v) sucrose, pH 7.5 (TSS). Alternatively, the LNP's were optionally concentrated using 100 kDa Amicon spin filter and buffer exchanged using PD-10 desalting columns (GE) into TSS. The resulting mixture was then filtered using a 0.2 μm sterile filter. The final LNP was stored at 4° C. or −80° C. until further use.

### Example 1.3. In Vitro Transcription (“IVT”) of mRNA

Capped and polyadenylated mRNA containing N1-methyl pseudo-U was generated by in vitro transcription using a linearized plasmid DNA template and T7 RNA polymerase. Plasmid DNA containing a T7 promoter, a sequence for transcription, and a polyadenylation sequence was linearized by incubating at 37° C. for 2 hours with XbaI with the following conditions: 200 ng/μL plasmid, 2 U/μL XbaI (NEB), and 1× reaction buffer. The XbaI was inactivated by heating the reaction at 65° C. for 20 minutes. The linearized plasmid was purified from enzyme and buffer salts. The IVT reaction to generate modified mRNA was performed by incubating at 37° C. for 1.5-4 hours in the following conditions: 50 ng/μL linearized plasmid; 2-5 mM each of GTP, ATP, CTP, and N1-methyl pseudo-UTP (Trilink); 10-25 mM ARCA (Trilink); 5 U/μL T7 RNA polymerase (NEB); 1 U/μL Murine RNase inhibitor (NEB); 0.004 U/μL Inorganic E. coli pyrophosphatase (NEB); and 1× reaction buffer. TURBO DNase (ThermoFisher) was added to a final concentration of 0.01 U/μL, and the reaction was incubated for an additional 30 minutes to remove the DNA template. The mRNA was purified using a MegaClear Transcription Clean-up kit (ThermoFisher) or a RNeasy Maxi kit (Qiagen) per the manufacturers' protocols. Alternatively, the mRNA was purified through a precipitation protocol, which in some cases was followed by HPLC-based purification. Briefly, after the DNase digestion, mRNA is purified using LiCl precipitation, ammonium acetate precipitation and sodium acetate precipitation. For HPLC purified mRNA, after the LiCl precipitation and reconstitution, the mRNA was purified by RP-IP HPLC (see, e.g., Kariko, et al. Nucleic Acids Research, 2011, Vol. 39, No. 21 e142). The fractions chosen for pooling were combined and desalted by sodium acetate/ethanol precipitation as described above. In a further alternative method, mRNA was purified with a LiCl precipitation method followed by further purification by tangential flow filtration. RNA concentrations were determined by measuring the light absorbance at 260 nm (Nanodrop), and transcripts were analyzed by capillary electrophoresis by Bioanlayzer (Agilent).

Streptococcus pyogenes (“Spy”) Cas9 mRNA was generated from plasmid DNA encoding an open reading frame according to SEQ ID NO: 307 (see sequences in Table of Sequences). Sp BC22n mRNA was generated from plasmid DNA encoding an open reading frame according to SEQ ID NO: 306. UGI mRNA was generated from plasmid DNA encoding an open reading frame according to SEQ ID NOs: 309. Nesseria meningitidis (Nme2) Cas9 mRNA was generated from plasmid DNA encoding an open reading frame according to SEQ ID NO: 305. Nme2 BC22n mRNA was generated from plasmid DNA encoding an open reading frame according to SEQ ID NO: 308. With respect to RNAs, it is understood that Ts should be replaced with Us (which were N1-methyl pseudouridines as described above). Messenger RNAs used in the Examples include a 5′ cap and a 3′ polyadenylation region, e.g., up to 100 nts, and are described, for example, in SEQ ID NO: 147 in Table of Sequences. Guide RNAs were chemically synthesized by methods known in the art.

### Example 2. One Pot Methods Using Electroporation

A solution containing a mixture of corresponding mRNAs encoding either SpBC22n (SEQ ID NO: 306) and UGI (SEQ ID NO: 309) or Nme2BC22n (SEQ ID NO: 308) and UGI (SEQ ID NO: 309) with or without Spy Cas9 (SEQ ID NO: 307) or Nme2 Cas9 (SEQ ID NO: 305) mRNAs was prepared in P3 buffer. Each guide used in this study was initially heat denatured at 95° C. for 2 minutes followed by 5 minute incubation at room temperature and cooled on ice. Healthy human donor apheresis was obtained commercially (Hemacare). T cells were isolated by negative selection using the EasySep Human T cell Isolation Kit (Stemcell Technology, Cat. 17951) following manufacturer's instruction. T cells were cryopreserved in Cryostor CS10 freezing media (Stemcell, Cat. 07930) for future use. Beginning of this study (Day 0), cryopreserved T cells were thawed and cultured overnight in 1× cytokine T cell growth media consisting of CTS OpTmizer SFM (Gibco, A3705001) with IL-15 (5 ng/mL), IL-7 (5 ng/mL), and IL-2 (200 U/mL). Following day, T cells were activated through Transact (Miltenyi, Cat. 130-111-160).

Forty-eight hours post activation, T cells were harvested, centrifuged, and resuspended in P3 electroporation buffer (Lonza). For each well to be electroporated, 1×10\hat{ }5 T cells were mixed reagents as indicated in Tables 6 and 7. Where indicated, samples received 160 ng of mRNA encoding a Cas9 or base editor (BE), 160 ng of mRNA encoding UGI and 2 uM of each sgRNA in a final volume of 20 μL of P3 electroporation buffer. This mixture was electroporated using the manufacturer's pulse code. Electroporated T cells were immediately rested in T cell basal media without cytokines for 10 minutes before being washed and resuspended in 100 μL of T cell basal media with with IL15 (5 ng/mL), IL7 (5 ng/mL), and IL2 (200 U/mL) and with 0.5 uM Compound 1.

Compound 1 is a small molecule inhibitor of DNA-dependent protein kinase. The inhibitor is 9-(4,4-difluorocyclohexyl)-7-methyl-2-((7-methyl-[1,2,4]triazolo[1,5-a]pyridin-6-yl)amino)-7,9-dihydro-8H-purin-8-one, also depicted as:

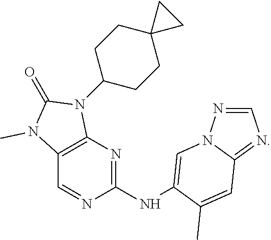 [SMILES: Cc1cc2ncnn2cc1Nc1ncc2c(n1)n(C1CCC3(CC1)CC3)c(=O)n2C]

DNAPKI Compound 1 was prepared as follows:

### General Information

All reagents and solvents were purchased and used as received from commercial vendors or synthesized according to cited procedures. All intermediates and final compounds were purified using flash column chromatography on silica gel. NMR spectra were recorded on a Bruker or Varian 400 MHz spectrometer, and NMR data were collected in CDCl3 at ambient temperature. Chemical shifts are reported in parts per million (ppm) relative to CDCl3 (7.26). Data for 1H NMR are reported as follows: chemical shift, multiplicity (br=broad, s=singlet, d=doublet, t=triplet, q=quartet, dd=doublet of doublets, dt=doublet of triplets m=multiplet), coupling constant, and integration. MS data were recorded on a Waters SQD2 mass spectrometer with an electrospray ionization (ESI) source. Purity of the final compounds was determined by UPLC-MS-ELS using a Waters Acquity H-Class liquid chromatography instrument equipped with SQD2 mass spectrometer with photodiode array (PDA) and evaporative light scattering (ELS) detectors.

### Intermediate 1a: (E)-N,N-dimethyl-N′-(4-methyl-5-nitropyridin-2-yl)formimidamide

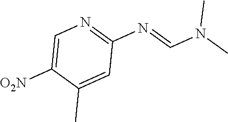

To a solution of 4-methyl-5-nitro-pyridin-2-amine (5 g, 1.0 equiv.) in toluene (0.3 M) was added DMF-DMA (3.0 equiv.). The mixture was stirred at 110° C. for 2 h. The reaction mixture was concentrated under reduced pressure to give a residue and purified by column chromatography to afford product as a yellow solid (59%). ¹H NMR (400 MHz, (CD₃)₂SO) δ 8.82 (s, 1H), 8.63 (s, 1H), 6.74 (s, 1H), 3.21 (m, 6H).

### Intermediate 1b: (E)-N-hydroxy-N′-(4-methyl-5-nitropyridin-2-yl)formimidamide

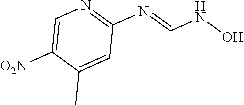

To a solution of Intermediate 1a (4 g, 1.0 equiv.) in MeOH (0.2 M) was added NH₂OH·HCl (2.0 equiv.). The reaction mixture was stirred at 80° C. for 1 h. The reaction mixture was filtered, and the filtrate was concentrated under reduced pressure to give a residue. The residue was partitioned between H₂O and EtOAc, followed by 2× extraction with EtOAc. The organic phases were concentrated under reduced pressure to give a residue and purified by column chromatography to afford product as a white solid (66%). 1H NMR (400 MHz, (CD₃)₂SO) δ 10.52 (d, J=3.8 Hz, 1H), 10.08 (dd, J=9.9, 3.7 Hz, 1H), 8.84 (d, J=3.8 Hz, 1H), 7.85 (dd, J=9.7, 3.8 Hz, 1H), 7.01 (d, J=3.9 Hz, 1H), 3.36 (s, 3H).

### Intermediate 1c: 7-methyl-6-nitro-[1,2,4]triazolo[1,5-a]pyridine

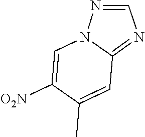

To a solution of Intermediate 1b (2.5 g, 1.0 equiv.) in THF (0.4 M) was added trifluoroacetic anhydride (1.0 equiv.) at 0° C. The mixture was stirred at 25° C. for 18 h. The reaction mixture was filtered, and the filtrate was concentrated under reduced pressure to give a residue. The residue was purified by column chromatography to afford product as a white solid (44%). ¹H NMR (400 MHz, CDCl3) δ 9.53 (s, 1H), 8.49 (s, 1H), 7.69 (s, 1H), 2.78 (d, J=1.0 Hz, 3H).

### Intermediate 1d: 7-methyl-[1,2,4]triazolo[1,5-a]pyridin-6-amine

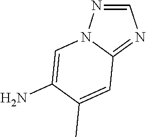

To a mixture of Pd/C (10% w/w, 0.2 equiv.) in EtOH (0.1 M) was added Intermediate 1c (1.0 equiv. and ammonium formate (5.0 equiv.). The mixture was heated at 105° C. for 2 h. The reaction mixture was filtered, and the filtrate was concentrated under reduced pressure to give a residue. The residue was purified by column chromatography to afford product as a pale brown solid. ¹H NMR (400 MHz, (CD₃)₂SO) δ 8.41 (s, 2H), 8.07 (d, J=9.0 Hz, 2H), 7.43 (s, 1H), 2.22 (s, 3H).

### Intermediate 1e: 8-methylene-1,4-dioxaspiro[4.5]decane

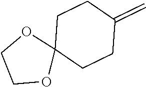

To a solution of methyl(triphenyl)phosphonium bromide (1.15 equiv.) in THF (0.6 M) was added n-BuLi (1.1 equiv.) at −78° C. dropwise, and the mixture was stirred at 0° C. for 1 h. Then, 1,4-dioxaspiro[4.5]decan-8-one (50 g, 1.0 equiv.) was added to the reaction mixture. The mixture was stirred at 25° C. for 12 h. The reaction mixture was poured into aq. NH₄Cl at 0° C., diluted with H₂O, and extracted 3× with EtOAc. The combined organic layers were concentrated under reduced pressure to give a residue and purified by column chromatography to afford product as a colorless oil (51%). ¹H NMR (400 MHz, CDCl₃) δ 4.67 (s, 1H), 3.96 (s, 4H), 2.82 (t, J=6.4 Hz, 4H), 1.70 (t, J=6.4 Hz, 4H).

### Intermediate 1f: 7,10-dioxadispiro[2.2.4⁶.2³]dodecane

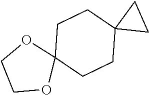 [SMILES: C1COC2(CCC3(CC3)CC2)O1]

To a solution of Intermediate 4a (5 g, 1.0 equiv.) in toluene (3 M) was added ZnEt2 (2.57 equiv.) dropwise at −40° C. and the mixture was stirred at −40° C. for 1 h. Then diiodomethane (6.0 equiv.) was added dropwise to the mixture at −40° C. under N2. The mixture was then stirred at 20° C. for 17 h under N₂ atmosphere. The reaction mixture was poured into aq. NH₄Cl at 0° C. and extracted 2× with EtOAc. The combined organic phases were washed with brine (20 mL), dried with anhydrous Na₂SO₄, filtered, and the filtrate was concentrated in vacuum. The residue was purified by column chromatography to afford product as a pale yellow oil (73%).

### Intermediate 1g: spiro[2.5]octan-6-one

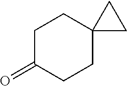

To a solution of Intermediate 4b (4 g, 1.0 equiv.) in 1:1 THF/H₂O (1.0 M) was added TFA (3.0 equiv.). The mixture was stirred at 20° C. for 2 h under N₂ atmosphere. The reaction mixture was concentrated under reduced pressure to remove THF, and the residue adjusted pH to 7 with 2 M NaOH (aq.). The mixture was poured into water and 3× extracted with EtOAc. The combined organic phase was washed with brine, dried with anhydrous Na₂SO₄, filtered, and the filtrate was concentrated in vacuum. The residue was purified by column chromatography to afford product as a pale yellow oil (68%). ¹H NMR (400 MHz, CDCl₃) δ 2.35 (t, J=6.6 Hz, 4H), 1.62 (t, J=6.6 Hz, 4H), 0.42 (s, 4H).

### Intermediate 1h: N-(4-methoxybenzyl)spiro[2.5]octan-6-amine

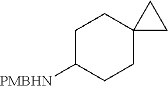

To a mixture of Intermediate 4c (2 g, 1.0 equiv.) and (4-methoxyphenyl)methanamine (1.1 equiv.) in DCM (0.3 M) was added AcOH (1.3 equiv.). The mixture was stirred at 20° C. for 1 h under N₂ atmosphere. Then, NaBH(OAc)₃ (3.3 equiv.) was added to the mixture at 0° C., and the mixture was stirred at 20° C. for 17 h under N₂ atmosphere. The reaction mixture was concentrated under reduced pressure to remove DCM, and the resulting residue was diluted with H₂O and extracted 3× with EtOAc. The combined organic layers were washed with brine, dried over Na₂SO₄, filtered, and the filtrate was concentrated under reduced pressure to give a residue. The residue was purified by column chromatography to afford product as a gray solid (51%). ¹H NMR (400 MHz, (CD₃)₂SO) δ 7.15-7.07 (m, 2H), 6.77-6.68 (m, 2H), 3.58 (s, 3H), 3.54 (s, 2H), 2.30 (ddt, J=10.1, 7.3, 3.7 Hz, 1H), 1.69-1.62 (m, 2H), 1.37 (td, J=12.6, 3.5 Hz, 2H), 1.12-1.02 (m, 2H), 0.87-0.78 (m, 2H), 0.13-0.04 (m, 2H).

### Intermediate 1i: spiro[2.5]octan-6-amine

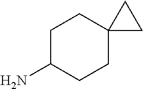

To a suspension of Pd/C (10% w/w, 1.0 equiv.) in MeOH (0.25 M) was added Intermediate 4d (2 g, 1.0 equiv.) and the mixture was stirred at 80° C. at 50 Psi for 24 h under H₂ atmosphere. The reaction mixture was filtered, and the filtrate was concentrated under reduced pressure to give a residue that was purified by column chromatography to afford product as a white solid. ¹H NMR (400 MHz, (CD₃)₂SO) δ 2.61 (tt, J=10.8, 3.9 Hz, 1H), 1.63 (ddd, J=9.6, 5.1, 2.2 Hz, 2H), 1.47 (td, J=12.8, 3.5 Hz, 2H), 1.21-1.06 (m, 2H), 0.82-0.72 (m, 2H), 0.14-0.05 (m, 2H).

### Intermediate 1j: ethyl 2-chloro-4-(spiro[2.5]octan-6-ylamino)pyrimidine-5-carboxylate

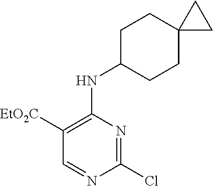 [SMILES: CCOC(=O)c1cnc(Cl)nc1NC1CCC2(CC1)CC2]

To a mixture of ethyl 2,4-dichloropyrimidine-5-carboxylate (2.7 g, 1.0 equiv.) and Intermediate 1i (1.0 equiv.) in ACN (0.5-0.6 M) was added K₂CO₃ (2.5 equiv.) in one portion under N₂. The mixture was stirred at 20° C. for 12 h. The reaction mixture was filtered, and the filtrate was concentrated under reduced pressure to give a residue. The residue was purified by column chromatography to afford product as a white solid (54%). ¹H NMR (400 MHz, (CD₃)₂SO) δ 8.64 (s, 1H), 8.41 (d, J=7.9 Hz, 1H), 4.33 (q, J=7.1 Hz, 2H), 4.08 (d, J=9.8 Hz, 1H), 1.90 (dd, J=12.7, 4.8 Hz, 2H), 1.64 (t, J=12.3 Hz, 2H), 1.52 (q, J=10.7, 9.1 Hz, 2H), 1.33 (t, J=7.1 Hz, 3H), 1.12 (d, J=13.0 Hz, 2H), 0.40-0.21 (m, 4H).

### Intermediate 1k: 2-chloro-4-(spiro[2.5]octan-6-ylamino)pyrimidine-5-carboxylic acid

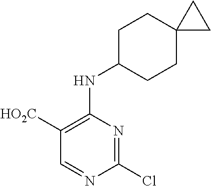 [SMILES: O=C(O)c1cnc(Cl)nc1NC1CCC2(CC1)CC2]

To a solution of Intermediate 1j (2 g, 1.0 equiv.) in 1:1 THF/H₂O (0.3 M) was added LiOH (2.0 equiv.). The mixture was stirred at 20° C. for 12 h. The reaction mixture was filtered, and the filtrate was concentrated under reduced pressure to give a residue. The residue was adjusted to pH 2 with 2 M HCl, and the precipitate was collected by filtration, washed with water, and tried under vacuum. Product was used directly in the next step without additional purification (82%). ¹H NMR (400 MHz, (CD₃)₂SO) δ 13.54 (s, 1H), 8.38 (d, J=8.0 Hz, 1H), 8.35 (s, 1H), 3.82 (qt, J=8.2, 3.7 Hz, 1H), 1.66 (dq, J=12.8, 4.1 Hz, 2H), 1.47-1.34 (m, 2H), 1.33-1.20 (m, 2H), 0.86 (dt, J=13.6, 4.2 Hz, 2H), 0.08 (dd, J=8.3, 4.8 Hz, 4H).

### Intermediate 1l: 2-chloro-9-(spiro[2.5]octan-6-yl)-7,9-dihydro-8H-purin-8-one

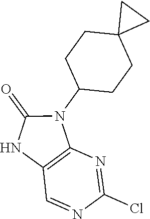 [SMILES: O=c1[nH]c2cnc(Cl)nc2n1C1CCC2(CC1)CC2]

To a mixture of Intermediate 1k (1.5 g, 1.0 equiv.) and Et₃N (1.0 equiv.) in DMF (0.3 M) was added DPPA (1.0 equiv.). The mixture was stirred at 120° C. for 8 h under N₂ atmosphere. The reaction mixture was poured into water. The precipitate was collected by filtration, washed with water, and dried under vacuum to give a residue that was used directly in the next step without additional purification (67%). ¹H NMR (400 MHz, (CD₃)₂SO) δ 11.68 (s, 1H), 8.18 (s, 1H), 4.26 (ddt, J=12.3, 7.5, 3.7 Hz, 1H), 2.42 (qd, J=12.6, 3.7 Hz, 2H), 1.95 (td, J=13.3, 3.5 Hz, 2H), 1.82-1.69 (m, 2H), 1.08-0.95 (m, 2H), 0.39 (tdq, J=11.6, 8.7, 4.2, 3.5 Hz, 4H).

### Intermediate 1m: 2-chloro-7-methyl-9-(spiro[2.5]octan-6-yl)-7,9-dihydro-8H-purin-8-one

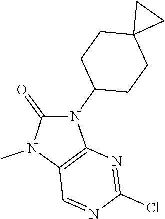 [SMILES: Cn1c(=O)n(C2CCC3(CC2)CC3)c2nc(Cl)ncc21]

To a mixture of Intermediate 1l (1.0 g, 1.0 equiv.) and NaOH (5.0 equiv.) in 1:1 THF/H₂O (0.3-0.5 M) was added Mel (2.0 equiv.). The mixture was stirred at 20° C. for 12 h under N₂ atmosphere. The reaction mixture was concentrated under reduced pressure to afford a residue that was purified by column chromatography to afford product as a pale yellow solid (67%). ¹H NMR (400 MHz, CDCl₃) δ 7.57 (s, 1H), 4.03 (tt, J=12.5, 3.9 Hz, 1H), 3.03 (s, 3H), 2.17 (qd, J=12.6, 3.8 Hz, 2H), 1.60 (td, J=13.4, 3.6 Hz, 2H), 1.47-1.34 (m, 2H), 1.07 (s, 1H), 0.63 (dp, J=14.0, 2.5 Hz, 2H), −0.05 (s, 4H).

### DNAPKI Compound 4: 7-methyl-2-((7-methyl-[1,2,4]triazolo[1,5-a]pyridin-6-yl)amino)-9-(spiro[2.5]octan-6-yl)-7,9-dihydro-8H-purin-8-one

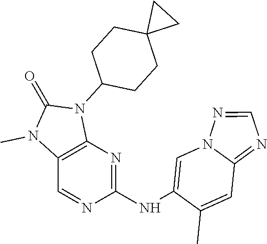 [SMILES: Cc1cc2ncnn2cc1Nc1ncc2c(n1)n(C1CCC3(CC1)CC3)c(=O)n2C]

To a mixture of Intermediate 1m (1.0 equiv.) and Intermediate 1d (1.0 equiv.), Pd(dppf)Cl₂ (0.2 equiv.), XantPhos (0.4 equiv.), and Cs₂CO₃ (2.0 equiv.) in DMF (0.2-0.3 M) was degassed and purged 3× with N₂, and the mixture was stirred at 130° C. for 12 h under N₂ atmosphere. The mixture was then poured into water and extracted 3× with DCM. The combined organic phase was washed with brine, dried over Na₂SO₄, filtered, and the filtrate was concentrated in vacuum. The residue was purified by column chromatography to afford product as an off-white solid. ¹H NMR (400 MHz, (CD₃)₂SO) δ 9.09 (s, 1H), 8.73 (s, 1H), 8.44 (s, 1H), 8.16 (s, 1H), 7.78 (s, 1H), 4.21 (t, J=12.5 Hz, 1H), 3.36 (s, 3H), 2.43 (s, 3H), 2.34 (dt, J=13.0, 6.5 Hz, 2H), 1.93-1.77 (m, 2H), 1.77-1.62 (m, 2H), 0.91 (d, J=13.2 Hz, 2H), 0.31 (t, J=7.1 Hz, 2H). MS: 405.5 m/z [M+H].

Cells treated with AAV received 3×10\hat{ }5 multiplicity of infection (MOI) of AAV6 encoding a TCR flanked by homology arms designed to the SpCas9 G013006 cut site (SEQ ID NO: 297). On the following day, an additional 100 μL of T cell basal media with cytokines were added to the T cells. Electroporated T cells were subsequently cultured for 4 additional days and cell pellets were collected for NGS sequencing as described in Example 1. On day 10 post-thaw, T cells were phenotyped by flow cytometry to determine if editing resulted in loss of cell surface proteins.

TABLE 6
Editing treatments

| Treatment | sgRNA | mRNA |
|---|---|---|
| Nme2 Cas9 | G021469 TRAC | SEQ ID No: 305 |
| Sp Cas9 | G013006 TRAC | SEQ ID No.: 307 |
| Nme2 Base | G028935 TRAC | Base editor SEQ ID No: 308 |
| Editor | G028986 TRBC | UGI SEQ ID No: 309 |
| | G028907 HLA-A | |
| Sp Base | G023520 TRAC | Base editor SEQ ID No: 306 |
| Editor | G023524 TRBC | UGI SSEQ ID No: 309 |
| | G023523 HLA-A | |

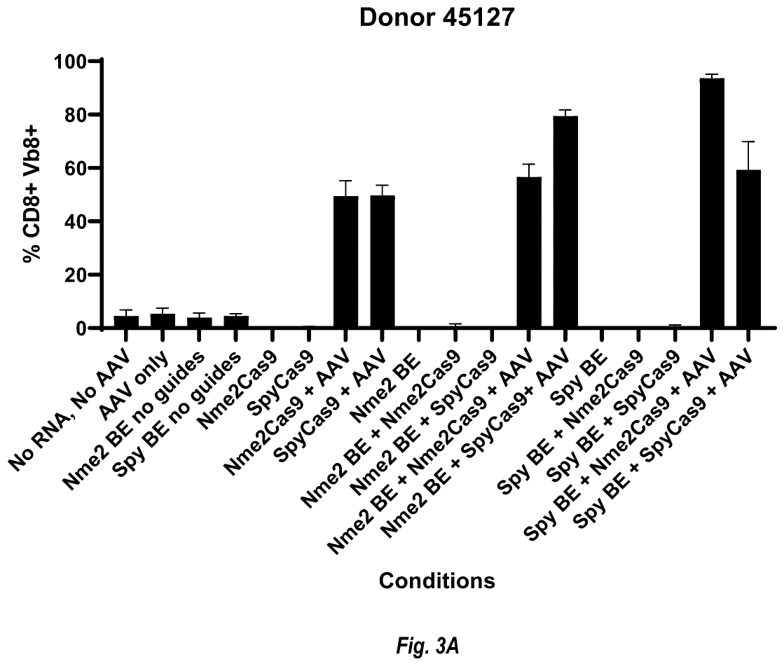

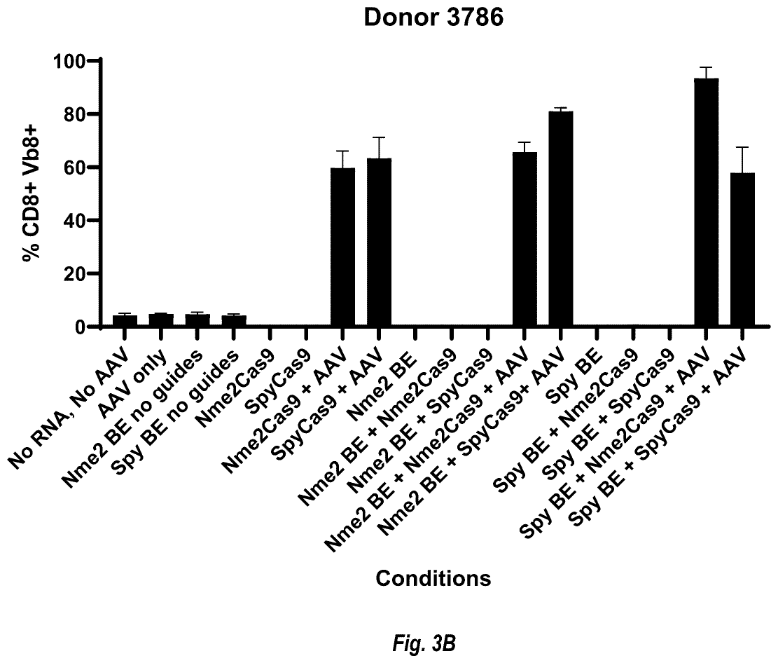

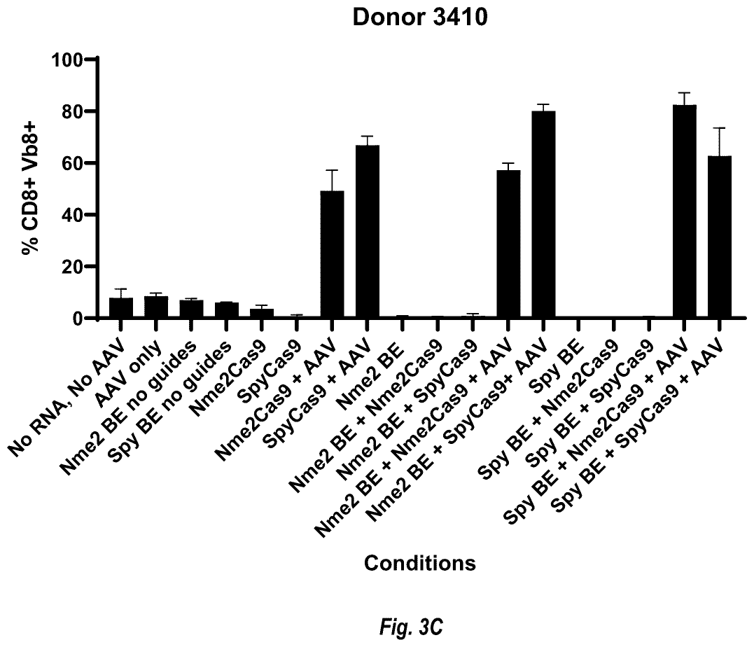

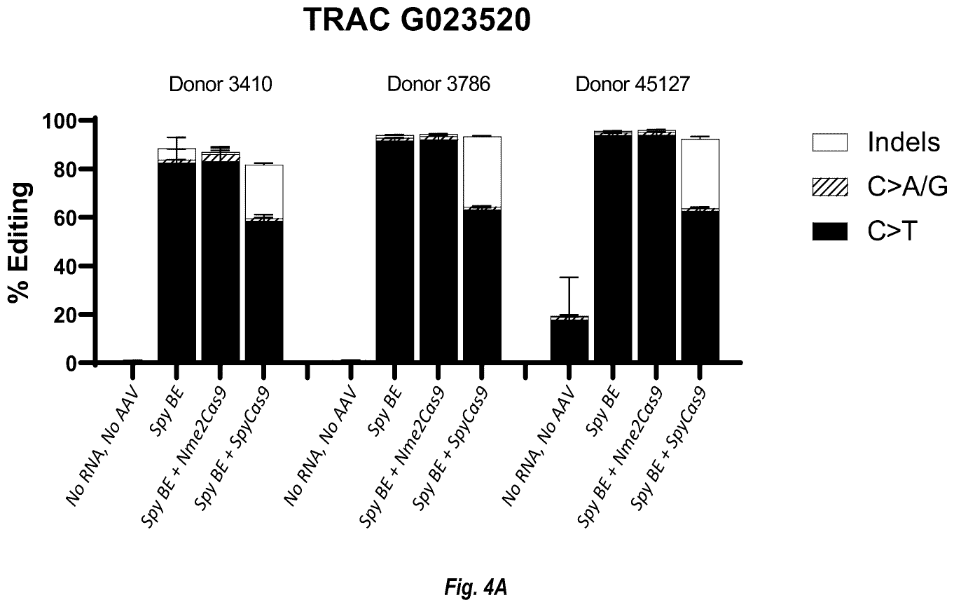

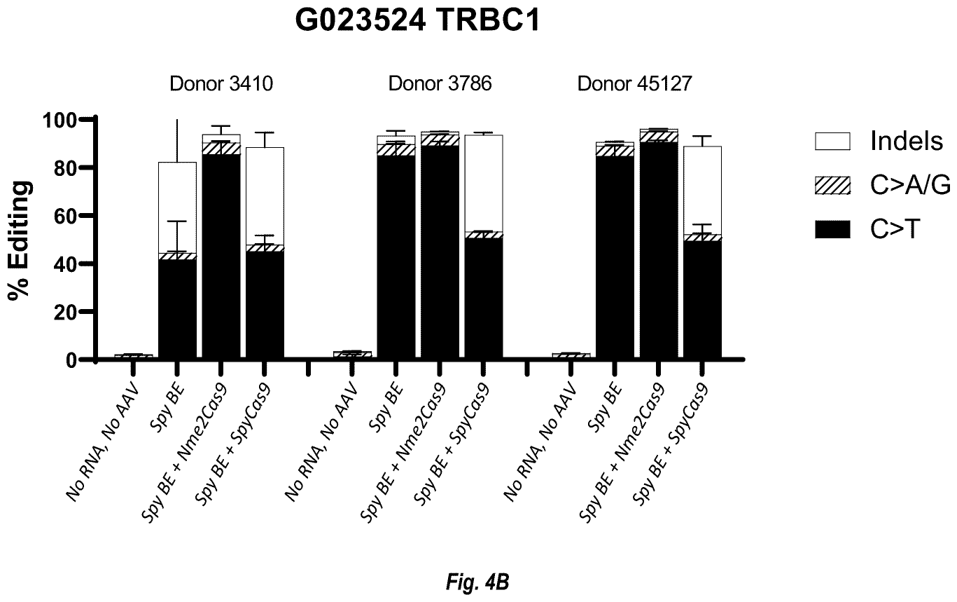

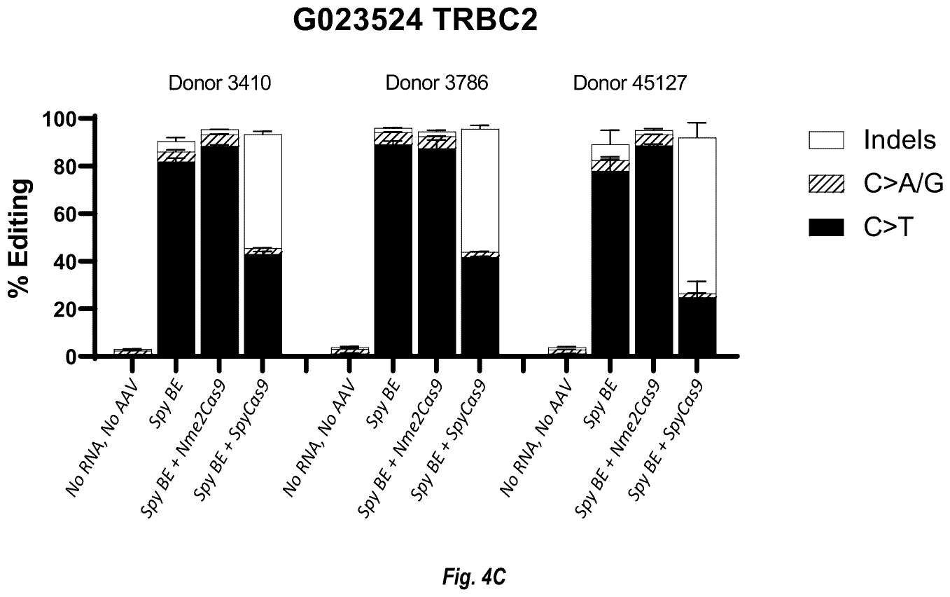

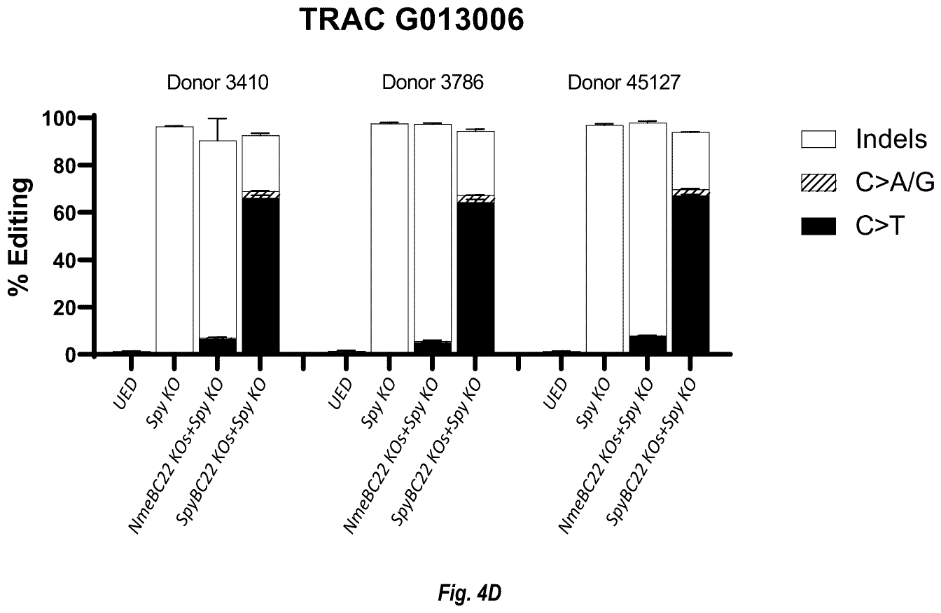

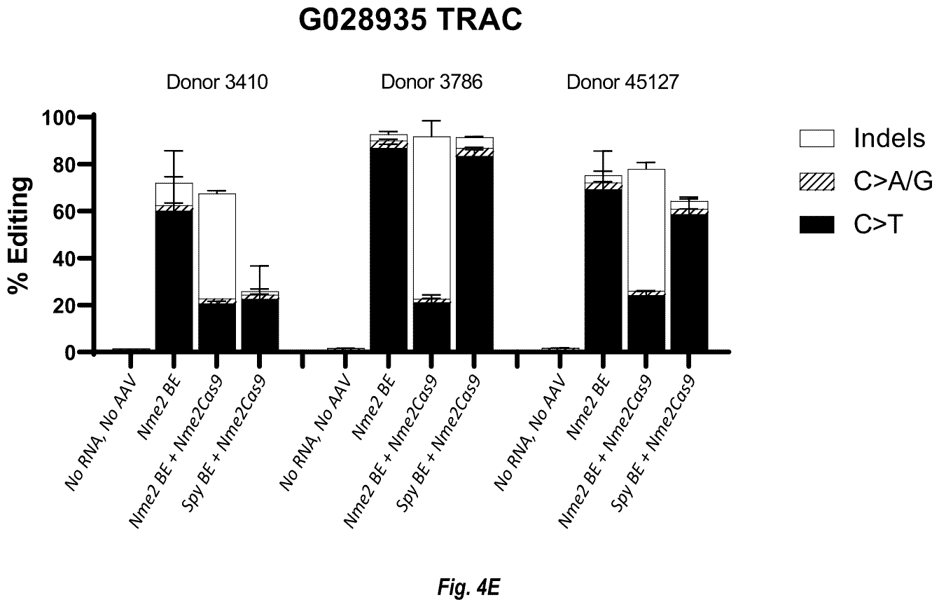

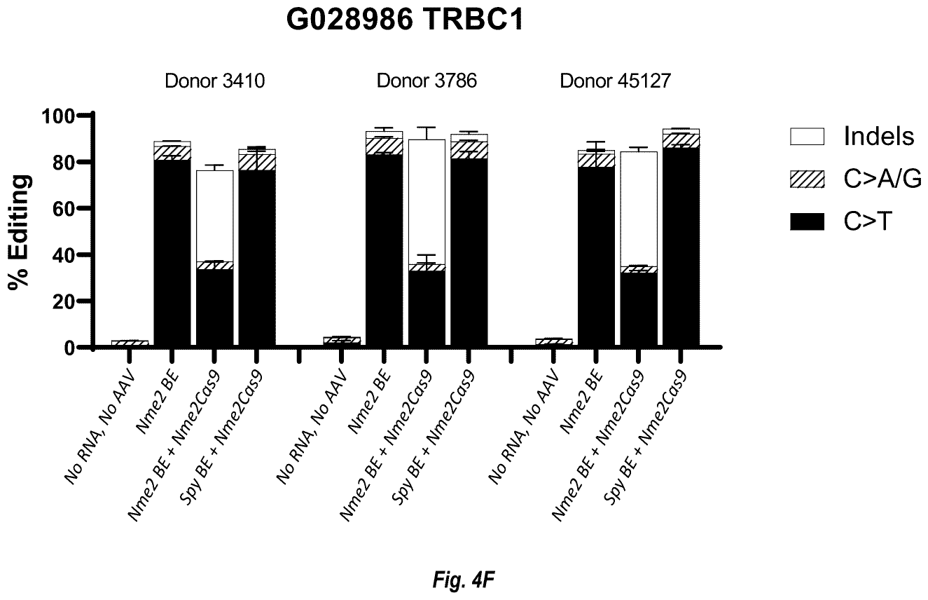

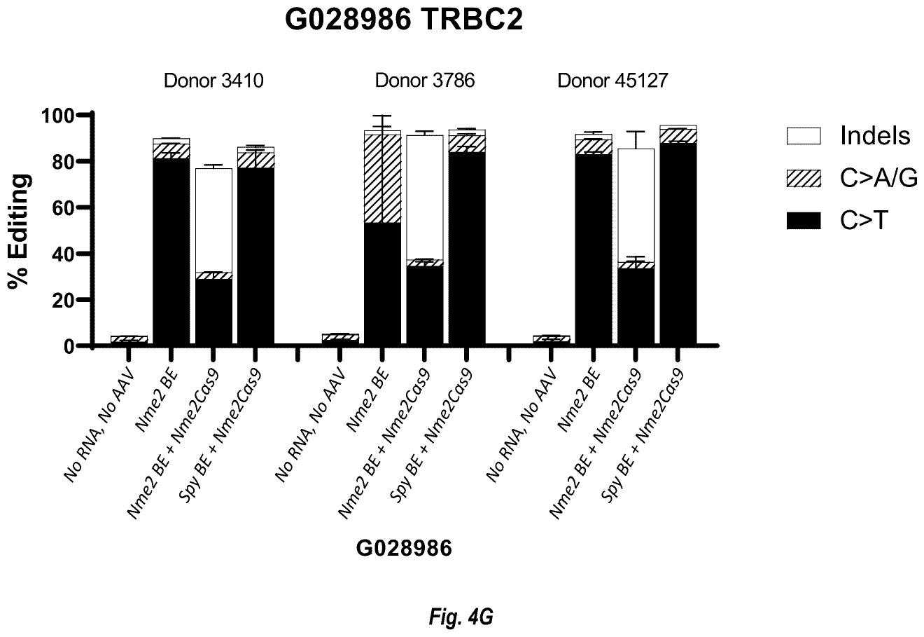

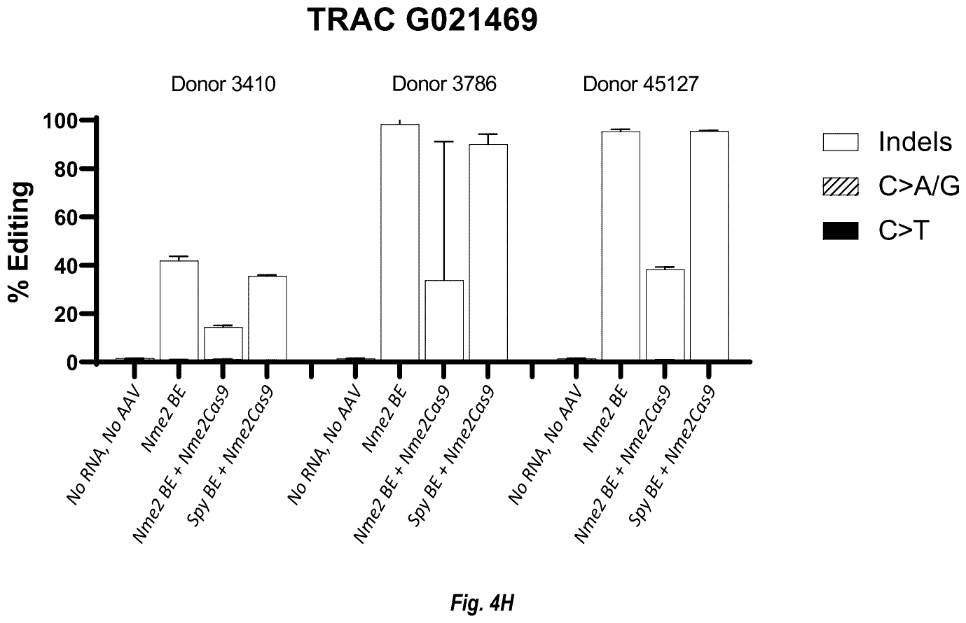

For flow cytometric analysis, cells were washed in FACS buffer (PBS+2% FBS+2 mM EDTA). Engineered T cells were incubated in a cocktail of antibodies targeting HLA-A2 (Biolegend, 343320), CD3 (Biolegend, 300430), CD4 (Biolegend, 317434), CD8 (Biolegend, 301046), Vb8 (Biolegend, 348104) and ViaKrome 808 Fixable Viability Dye (Beckman Coulter, C36628). T cells were subsequently washed and analyzed on a Cytoflex instrument (Beckman Coulter). Data analysis was performed using FlowJo software package (v.10.6.1). T cells were gated on size, viability, CD4 or CD8 expression, and expression of markers indicated in Table 7. Flow cytometry data in Table 7 and FIGS. 1A-2C show all cells with disrupted endogenous TRAC, TRBC1 or TRBC2 loci, represented as the percentage of cells that are not CD₃+Vb8−. Similarly, flow cytometry data in Table 7 and FIGS. 2A-2C show base editing that has disrupted HLA-A2 expression. The percentage of cells expressing the transgenic TCR (Vb8+) are shown in Table 7 and FIGS. 3A-3C. The results of the NGS data shown in Table 8 and FIGS. 4A-4H also show editing in TRAC, TRBC1 and TRBC2 loci when orthogonal Cas9 species were used.

TABLE 7
Mean percent of T cells displaying cell surface phenotype

| | | Donor 45127 | | Donor 3410 | | Donor 3786 | |
|---|---|---|---|---|---|---|---|
| Marker | Treatment | Mean | SD | Mean | SD | Mean | SD |
| HLA-A- | No RNA, No AAV | 3.11 | 1.01 | 2.41 | 0.77 | 0.31 | 0.32 |
| | AAV only | 1.28 | 1.33 | 3.52 | 2.66 | 0.50 | 0.18 |
| | Nme2 BE no guides | 0.58 | 0.39 | 5.73 | 2.02 | 0.18 | 0.16 |
| | Spy BE no guides | 0.64 | 0.47 | 1.46 | 1.04 | 0.19 | 0.16 |
| | Nme2Cas9 | 3.32 | 3.22 | 2.55 | 2.51 | 0.35 | 0.31 |
| | SpyCas9 | 3.28 | 3.75 | 2.43 | 1.16 | 0.37 | 0.31 |
| | Nme2Cas9 + AAV | 5.48 | 5.45 | 1.62 | 1.31 | 0.00 | 0.00 |
| | SpyCas9 + AAV | 3.94 | 4.05 | 1.26 | 1.64 | 0.28 | 0.49 |
| | Nme2 BE | 91.18 | 3.72 | 85.81 | 3.25 | 87.66 | 2.35 |
| | Nme2 BE + Nme2Cas9 | 86.95 | 4.04 | 76.65 | 0.97 | 85.61 | 2.58 |
| | Nme2 BE + SpyCas9 | 92.14 | 1.76 | 77.24 | 12.68 | 84.80 | 1.98 |
| | Nme2 BE + Nme2Cas9 + AAV | 96.17 | 0.86 | 89.47 | 3.07 | 91.14 | 2.99 |
| | Nme2 BE + SpyCas9 + AAV | 96.83 | 0.52 | 94.57 | 2.28 | 91.23 | 0.66 |
| | Spy BE | 96.17 | 0.35 | 93.77 | 2.33 | 96.37 | 0.72 |
| | Spy BE + Nme2Cas9 | 96.85 | 0.86 | 89.84 | 3.15 | 91.58 | 3.17 |
| | Spy BE + SpyCas9 | 88.39 | 3.19 | 90.08 | 2.81 | 85.05 | 4.59 |
| | Spy BE + Nme2Cas9 + AAV | 99.13 | 0.60 | 98.18 | 0.70 | 99.52 | 0.28 |
| | Spy BE + SpyCas9 + AAV | 95.90 | 3.57 | 94.33 | 6.33 | 85.08 | 15.10 |
| 100− | No RNA, No AAV | 5.43 | 1.57 | 8.77 | 4.76 | 4.90 | 0.53 |
| (CD3+ | AAV only | 5.47 | 2.10 | 8.93 | 1.12 | 5.59 | 0.19 |
| Vb8−) | Nme2 BE no guides | 4.95 | 0.87 | 7.37 | 0.87 | 5.20 | 0.80 |
| | Spy BE no guides | 5.06 | 0.68 | 6.30 | 0.28 | 4.60 | 0.71 |
| | Nme2Cas9 | 95.40 | 0.33 | 38.61 | 4.83 | 93.38 | 1.33 |
| | SpyCas9 | 95.69 | 1.07 | 96.87 | 1.86 | 95.40 | 1.21 |
| | Nme2Cas9 + AAV | 55.60 | 4.17 | 52.36 | 7.23 | 64.49 | 3.87 |
| | SpyCas9 + AAV | 55.21 | 3.59 | 70.55 | 2.78 | 68.82 | 6.50 |
| | Nme2 BE | 96.86 | 2.85 | 92.80 | 6.16 | 92.87 | 1.17 |
| | Nme2 BE + Nme2Cas9 | 93.62 | 2.26 | 89.74 | 2.27 | 93.45 | 3.39 |
| | Nme2 BE + SpyCas9 | 97.11 | 2.00 | 86.25 | 12.98 | 95.45 | 0.87 |
| | Nme2 BE + Nme2Cas9 + AAV | 96.07 | 1.53 | 97.14 | 0.56 | 97.75 | 1.08 |
| | Nme2 BE + SpyCas9 + AAV | 96.37 | 0.59 | 97.69 | 0.18 | 97.27 | 1.15 |
| | Spy BE | 97.84 | 1.13 | 97.01 | 1.20 | 96.03 | 0.20 |
| | Spy BE + Nme2Cas9 | 98.85 | 0.51 | 97.79 | 0.82 | 93.99 | 1.57 |
| | Spy BE + SpyCas9 | 92.26 | 3.62 | 92.97 | 2.36 | 85.78 | 5.33 |
| | Spy BE + Nme2Cas9 + AAV | 99.66 | 0.59 | 98.58 | 0.80 | 99.63 | 0.21 |
| | Spy BE + SpyCas9 + AAV | 95.39 | 4.32 | 94.63 | 6.43 | 86.04 | 14.58 |
| Vb8+ | No RNA, No AAV | 4.52 | 2.28 | 7.85 | 3.41 | 4.30 | 0.76 |
| | AAV only | 5.37 | 2.12 | 8.57 | 1.15 | 4.81 | 0.24 |
| | Nme2 BE no guides | 3.94 | 1.70 | 6.93 | 0.70 | 4.69 | 0.74 |
| | Spy BE no guides | 4.58 | 0.81 | 6.02 | 0.18 | 4.21 | 0.62 |
| | Nme2Cas9 | 0.28 | 0.16 | 3.59 | 1.46 | 0.23 | 0.23 |
| | SpyCas9 | 0.34 | 0.31 | 0.48 | 0.83 | 0.14 | 0.09 |
| | Nme2Cas9 + AAV | 49.49 | 5.74 | 49.23 | 8.00 | 59.70 | 6.46 |
| | SpyCas9 + AAV | 49.72 | 3.83 | 66.90 | 3.45 | 63.34 | 7.86 |
| | Nme2 BE | 0.05 | 0.09 | 0.35 | 0.61 | 0.13 | 0.07 |
| | Nme2 BE + Nme2Cas9 | 0.60 | 1.05 | 0.26 | 0.46 | 0.12 | 0.15 |
| | Nme2 BE + SpyCas9 | 0.22 | 0.19 | 0.90 | 0.90 | 0.05 | 0.04 |
| | Nme2 BE + Nme2Cas9 + AAV | 56.65 | 4.77 | 57.19 | 2.78 | 65.74 | 3.66 |
| | Nme2 BE + Spy Cas9 + AAV | 79.48 | 2.27 | 80.11 | 2.56 | 81.06 | 1.35 |
| | Spy BE | 0.06 | 0.05 | 0.33 | 0.13 | 0.09 | 0.02 |
| | Spy BE + Nme2Cas9 | 0.05 | 0.08 | 0.38 | 0.05 | 0.26 | 0.34 |
| | Spy BE + SpyCas9 | 0.58 | 0.60 | 0.42 | 0.26 | 0.11 | 0.19 |
| | Spy BE + Nme2Cas9 + AAV | 93.65 | 1.49 | 82.46 | 4.70 | 93.43 | 4.20 |
| | Spy BE + SpyCas9 + AAV | 59.29 | 10.60 | 62.74 | 10.82 | 57.84 | 9.70 |

TABLE 8
Mean percent editing in T cells

| | | | C to T | | | C to A/G | | | Indels | | |
|---|---|---|---|---|---|---|---|---|---|---|---|
| Locus | Donor | Treatment | Mean | SD | n | Mean | SD | n | Mean | SD | n |
| Spy BE | Donor | No RNA, No AAV | 0.22 | 0.21 | 3 | 0.47 | 0.41 | 3 | 0.14 | 0.13 | 3 |
| TRAC | 3410 | Spy BE | 82.54 | 5.70 | 3 | 1.18 | 0.07 | 3 | 4.71 | 4.56 | 3 |
| G023520 | | Spy BE + Nme2Cas9 | 83.08 | 6.03 | 3 | 2.92 | 2.64 | 3 | 0.89 | 0.78 | 3 |
| | | Spy BE + SpyCas9 | 58.58 | 2.58 | 3 | 1.04 | 0.27 | 3 | 22.05 | 0.76 | 3 |
| | Donor | No RNA, No AAV | 0.31 | 0.19 | 3 | 0.63 | 0.00 | 3 | 0.19 | 0.02 | 3 |
| | 3786 | Spy BE | 91.66 | 0.95 | 3 | 1.11 | 0.08 | 3 | 1.18 | 0.20 | 3 |
| | | Spy BE + Nme2Cas9 | 92.07 | 2.22 | 3 | 1.37 | 0.19 | 3 | 0.94 | 0.15 | 3 |
| | | Spy BE + SpyCas9 | 63.28 | 0.72 | 3 | 1.11 | 0.28 | 3 | 28.98 | 0.27 | 3 |
| | Donor | No RNA, No AAV | 17.82 | 17.45 | 3 | 1.00 | 0.29 | 3 | 0.55 | 0.48 | 3 |
| | 45127 | Spy BE | 93.89 | 0.94 | 3 | 1.08 | 0.12 | 3 | 0.63 | 0.11 | 3 |
| | | Spy BE + Nme2Cas9 | 94.05 | 1.12 | 3 | 1.14 | 0.09 | 3 | 0.71 | 0.24 | 3 |
| | | Spy BE + SpyCas9 | 62.68 | 1.62 | 3 | 1.00 | 0.09 | 3 | 28.64 | 0.96 | 3 |
| Spy BE | Donor | No RNA, No AAV | 0.30 | 0.02 | 3 | 1.58 | 0.17 | 3 | 0.32 | 0.12 | 3 |
| TRBC1 | 3410 | Spy BE | 41.64 | 16.02 | 3 | 2.78 | 0.72 | 3 | 37.83 | 26.97 | 3 |
| G023524 | | Spy BE + Nme2Cas9 | 85.43 | 5.60 | 3 | 4.94 | 0.40 | 3 | 3.36 | 3.54 | 3 |
| | | Spy BE + SpyCas9 | 45.07 | 6.66 | 3 | 2.77 | 0.33 | 3 | 40.63 | 6.14 | 3 |
| | Donor | No RNA, No AAV | 1.31 | 0.93 | 3 | 1.70 | 0.10 | 3 | 0.53 | 0.07 | 3 |
| | 3786 | Spy BE | 84.95 | 5.87 | 2 | 4.76 | 0.25 | 2 | 3.50 | 2.12 | 2 |
| | | Spy BE + Nme2Cas9 | 89.15 | 1.66 | 3 | 4.56 | 0.16 | 3 | 1.26 | 0.10 | 3 |
| | | Spy BE + SpyCas9 | 50.70 | 2.37 | 3 | 2.66 | 0.17 | 3 | 40.24 | 0.94 | 3 |
| | Donor | No RNA, No AAV | 0.59 | 0.05 | 3 | 1.58 | 0.28 | 3 | 0.51 | 0.09 | 3 |
| | 45127 | Spy BE | 84.82 | 4.62 | 3 | 4.20 | 0.45 | 3 | 1.55 | 0.23 | 3 |
| | | Spy BE + Nme2Cas9 | 90.59 | 0.74 | 3 | 4.36 | 0.30 | 3 | 1.12 | 0.08 | 3 |
| | | Spy BE + SpyCas9 | 49.44 | 6.79 | 3 | 2.74 | 0.43 | 3 | 36.66 | 4.32 | 3 |
| Spy BE | Donor | No RNA, No AAV | 1.82 | 0.56 | 3 | 2.22 | 0.18 | 3 | 0.33 | 0.02 | 3 |
| TRBC2 | 3410 | Nme2 BE | 81.25 | 2.37 | 3 | 6.25 | 0.27 | 3 | 2.38 | 0.17 | 3 |
| G023524 | | Nme2 BE + Nme2Cas9 | 28.93 | 2.87 | 3 | 2.90 | 0.16 | 3 | 45.04 | 1.57 | 3 |
| | | Spy BE + Nme2Cas9 | 77.18 | 7.55 | 3 | 6.58 | 1.17 | 3 | 2.37 | 0.68 | 3 |
| | Donor | No RNA, No AAV | 2.58 | 0.26 | 2 | 2.23 | 0.00 | 2 | 0.39 | 0.05 | 2 |
| | 3786 | Nme2 BE | 53.46 | 46.30 | 3 | 38.01 | 53.69 | 3 | 1.83 | 1.70 | 3 |
| | | Nme2 BE + Nme2Cas9 | 34.59 | 1.87 | 3 | 2.77 | 0.25 | 3 | 53.90 | 1.70 | 3 |
| | | Spy BE + Nme2Cas9 | 84.02 | 2.41 | 3 | 7.17 | 0.61 | 3 | 2.43 | 0.51 | 3 |
| | Donor | No RNA, No AAV | 1.89 | 0.86 | 3 | 2.28 | 0.09 | 3 | 0.29 | 0.10 | 3 |
| | 45127 | Nme2 BE | 83.00 | 0.98 | 3 | 6.36 | 0.29 | 3 | 2.39 | 0.94 | 3 |
| | | Nme2 BE + Nme2Cas9 | 33.53 | 5.09 | 3 | 2.78 | 0.32 | 3 | 49.17 | 7.44 | 3 |
| | | Spy BE + Nme2Cas9 | 87.88 | 0.75 | 3 | 5.97 | 0.28 | 3 | 1.77 | 0.04 | 3 |
| SpyCas9 | Donor | No RNA, No AAV | 0.16 | 0.03 | 3 | 0.73 | 0.06 | 3 | 0.44 | 0.11 | 3 |
| TRAC | 3410 | SpyCas9 | 0.01 | 0.01 | 3 | 0.13 | 0.03 | 3 | 96.11 | 0.27 | 3 |
| G013006 | | Nme2 BE + SpyCas9 | 6.54 | 0.68 | 3 | 0.45 | 0.13 | 3 | 83.37 | 9.38 | 3 |
| | | Spy BE + SpyCas9 | 66.13 | 1.10 | 3 | 2.77 | 0.23 | 3 | 23.59 | 0.94 | 3 |
| | Donor | No RNA, No AAV | 0.18 | 0.04 | 3 | 0.79 | 0.04 | 3 | 0.46 | 0.21 | 3 |
| | 3786 | SpyCas9 | 0.03 | 0.01 | 3 | 0.06 | 0.01 | 3 | 97.47 | 0.41 | 3 |
| | | Nme2 BE + SpyCas9 | 5.19 | 0.80 | 3 | 0.30 | 0.03 | 3 | 91.86 | 0.43 | 3 |
| | | Spy BE + SpyCas9 | 64.38 | 1.16 | 3 | 2.78 | 0.18 | 3 | 27.24 | 0.77 | 3 |
| | Donor | No RNA, No AAV | 0.22 | 0.04 | 3 | 0.76 | 0.16 | 3 | 0.32 | 0.08 | 3 |
| | 45127 | SpyCas9 | 0.03 | 0.02 | 3 | 0.11 | 0.03 | 3 | 96.72 | 0.54 | 3 |
| | | Nme2 BE + SpyCas9 | 7.57 | 0.50 | 3 | 0.33 | 0.01 | 3 | 89.96 | 0.66 | 3 |
| | | Spy BE + SpyCas9 | 67.08 | 0.48 | 3 | 2.69 | 0.33 | 3 | 24.13 | 0.13 | 3 |
| Nme2 BE | Donor | No RNA, No AAV | 0.24 | 0.00 | 1 | 1.18 | 0.00 | 1 | 0.13 | 0.00 | 1 |
| TRAC | 3410 | Nme2 BE | 60.14 | 14.46 | 3 | 2.24 | 1.07 | 3 | 9.63 | 13.65 | 3 |
| G028935 | | Nme2 BE + Nme2Cas9 | 20.74 | 0.91 | 2 | 2.03 | 0.01 | 2 | 44.67 | 1.31 | 2 |
| | | Spy BE + Nme2Cas9 | 22.62 | 14.01 | 3 | 1.78 | 0.32 | 3 | 1.45 | 0.99 | 3 |
| | Donor | No RNA, No AAV | 0.30 | 0.05 | 3 | 1.19 | 0.17 | 3 | 0.15 | 0.02 | 3 |
| | 3786 | Nme2 BE | 86.81 | 1.57 | 3 | 3.18 | 0.55 | 3 | 2.69 | 1.25 | 3 |
| | | Nme2 BE + Nme2Cas9 | 21.13 | 3.19 | 3 | 1.55 | 0.25 | 3 | 69.11 | 6.71 | 3 |
| | | Spy BE + Nme2Cas9 | 83.36 | 2.74 | 3 | 3.51 | 0.26 | 3 | 4.50 | 0.36 | 3 |
| | Donor | No RNA, No AAV | 0.34 | 0.04 | 2 | 1.28 | 0.13 | 2 | 0.10 | 0.01 | 2 |
| | 45127 | Nme2 BE | 69.26 | 16.30 | 3 | 2.86 | 0.43 | 3 | 3.04 | 1.83 | 3 |
| | | Nme2 BE + Nme2Cas9 | 24.18 | 1.65 | 3 | 1.85 | 0.16 | 3 | 51.95 | 2.75 | 3 |
| | | Spy BE + Nme2Cas9 | 58.64 | 6.57 | 3 | 2.29 | 0.06 | 3 | 3.31 | 1.68 | 3 |
| Nme2 BE | Donor | No RNA, No AAV | 0.67 | 0.02 | 3 | 2.14 | 0.06 | 3 | 0.19 | 0.02 | 3 |
| TRBC1 | 3410 | Nme2 BE | 80.76 | 1.87 | 3 | 5.96 | 0.12 | 3 | 2.19 | 0.06 | 3 |
| G028986 | | Nme2 BE + Nme2Cas9 | 33.74 | 3.05 | 3 | 3.25 | 0.22 | 3 | 39.30 | 2.37 | 3 |
| | | Spy BE + Nme2Cas9 | 76.48 | 10.05 | 3 | 6.76 | 1.27 | 3 | 2.18 | 0.38 | 3 |
| | Donor | No RNA, No AAV | 2.08 | 0.88 | 3 | 2.10 | 0.26 | 3 | 0.29 | 0.15 | 3 |
| | 3786 | Nme2 BE | 83.13 | 0.88 | 3 | 7.15 | 0.50 | 3 | 2.89 | 1.48 | 3 |
| | | Nme2 BE + Nme2Cas9 | 32.96 | 6.88 | 3 | 2.87 | 0.62 | 3 | 53.89 | 5.17 | 3 |
| | | Spy BE + Nme2Cas9 | 81.49 | 2.92 | 3 | 7.34 | 0.52 | 3 | 3.14 | 1.18 | 3 |
| | Donor | No RNA, No AAV | 1.30 | 0.17 | 3 | 2.23 | 0.24 | 3 | 0.23 | 0.01 | 3 |
| | 45127 | Nme2 BE | 77.77 | 10.90 | 3 | 5.68 | 1.10 | 3 | 1.60 | 0.30 | 3 |
| | | Nme2 BE + Nme2Cas9 | 32.22 | 0.92 | 3 | 2.76 | 0.30 | 3 | 49.49 | 1.83 | 3 |
| | | Spy BE + Nme2Cas9 | 86.16 | 1.21 | 3 | 5.91 | 0.29 | 3 | 2.21 | 0.14 | 3 |
| Nme2 BE | Donor | No RNA, No AAV | 1.82 | 0.56 | 3 | 2.22 | 0.18 | 3 | 0.33 | 0.02 | 3 |
| TRBC2 | 3410 | Nme2 BE | 81.25 | 2.37 | 3 | 6.25 | 0.27 | 3 | 2.38 | 0.17 | 3 |
| G028986 | | Nme2 BE + Nme2Cas9 | 28.93 | 2.87 | 3 | 2.90 | 0.16 | 3 | 45.04 | 1.57 | 3 |
| | | Spy BE + Nme2Cas9 | 77.18 | 7.55 | 3 | 6.58 | 1.17 | 3 | 2.37 | 0.68 | 3 |
| | Donor | No RNA, No AAV | 2.58 | 0.26 | 2 | 2.23 | 0.00 | 2 | 0.39 | 0.05 | 2 |
| | 3786 | Nme2 BE | 53.46 | 46.30 | 3 | 38.01 | 53.69 | 3 | 1.83 | 1.70 | 3 |
| | | Nme2 BE + Nme2Cas9 | 34.59 | 1.87 | 3 | 2.77 | 0.25 | 3 | 53.90 | 1.70 | 3 |
| | | Spy BE + Nme2Cas9 | 84.02 | 2.41 | 3 | 7.17 | 0.61 | 3 | 2.43 | 0.51 | 3 |
| | Donor | No RNA, No AAV | 1.89 | 0.86 | 3 | 2.28 | 0.09 | 3 | 0.29 | 0.10 | 3 |
| | 45127 | Nme2 BE | 83.00 | 0.98 | 3 | 6.36 | 0.29 | 3 | 2.39 | 0.94 | 3 |
| | | Nme2 BE + Nme2Cas9 | 33.53 | 5.09 | 3 | 2.78 | 0.32 | 3 | 49.17 | 7.44 | 3 |
| | | Spy BE + Nme2Cas9 | 87.88 | 0.75 | 3 | 5.97 | 0.28 | 3 | 1.77 | 0.04 | 3 |
| Nme2Cas9 | Donor | No RNA, No AAV | 0.17 | 0.03 | 3 | 0.75 | 0.14 | 3 | 0.57 | 0.09 | 3 |
| TRAC | 3410 | Nme2 BE | 0.10 | 0.01 | 3 | 0.63 | 0.23 | 3 | 41.21 | 1.82 | 3 |
| G021469 | | Nme2 BE + Nme2Cas9 | 0.37 | 0.02 | 3 | 0.73 | 0.07 | 3 | 13.32 | 0.64 | 3 |
| | | Spy BE + Nme2Cas9 | 0.10 | 0.02 | 3 | 0.55 | 0.04 | 3 | 34.91 | 0.39 | 3 |
| | Donor | No RNA, No AAV | 0.19 | 0.08 | 3 | 0.72 | 0.05 | 3 | 0.47 | 0.21 | 3 |
| | 3786 | Nme2 BE | 0.02 | 0.04 | 3 | 0.04 | 0.04 | 3 | 98.31 | 2.79 | 3 |
| | | Nme2 BE + Nme2Cas9 | 0.00 | 0.00 | 3 | 0.00 | 0.00 | 3 | 33.83 | 57.30 | 3 |
| | | Spy BE + Nme2Cas9 | 0.05 | 0.02 | 3 | 0.14 | 0.05 | 3 | 89.85 | 4.22 | 3 |
| | Donor | No RNA, No AAV | 0.21 | 0.05 | 3 | 0.78 | 0.08 | 3 | 0.41 | 0.10 | 3 |
| | 45127 | Nme2 BE | 0.03 | 0.01 | 3 | 0.08 | 0.01 | 3 | 95.24 | 0.85 | 3 |
| | | Nme2 BE + Nme2Cas9 | 0.27 | 0.07 | 3 | 0.50 | 0.07 | 3 | 37.49 | 1.05 | 3 |
| | | Spy BE + Nme2Cas9 | 0.04 | 0.01 | 3 | 0.11 | 0.02 | 3 | 95.29 | 0.22 | 3 |

### Example 3. One Pot Methods Using Lipid Nanoparticles

### Example 3.1. T Cell Preparation

Healthy human donor apheresis was obtained commercially (Hemacare), and cells were washed, re-suspended in CliniMACS® PBS/EDTA buffer (Miltenyi Biotec Cat. 130-070-525) and processed in a MultiMACS™ Cell 24 Separator Plus device (Miltenyi Biotec). T cells were isolated via positive selection using a Straight from Leukopak® CD4/CD8 MicroBead kit, human (Miltenyi Biotec Cat. 130-122-352). T cells were aliquoted and cryopreserved for future use in Cryostor® CS10 (StemCell Technologies Cat. 07930).

Upon thaw, T cells were plated at a density of 1.0×10\hat{ }6 cells/mL in T cell growth media (TCGM) composed of CTS OpTmizer T Cell Expansion SFM and T Cell Expansion Supplement (ThermoFisher Cat. A1048501), 5% human AB serum (GeminiBio, Cat. 100-512) 1× Penicillin-Streptomycin, 1× Glutamax, 10 mM HEPES, 200 U/mL recombinant human interleukin-2 (Peprotech, Cat. 200-02), 5 ng/mL recombinant human interleukin 7 (Peprotech, Cat. 200-07), and 5 ng/mL recombinant human interleukin 15 (Peprotech, Cat. 200-15). T cells were rested in this media for 24 hours, at which time they were activated with T Cell TransAct™, human reagent (Miltenyi, Cat. 130-111-160) added at a 1:100 ratio by volume. T cells were activated for 48 hours prior to LNP treatment.

### Example 3.2. T Cell Treatment and Expansion

Forty-eight hours post-activation, T cells were harvested, centrifuged at 500 g for 5 min, and resuspended at a concentration of 1×10\hat{ }6 T cells/mL in T cell plating media (TCPM): a serum-free version of TCGM containing 400 U/mL recombinant human interleukin-2 (Peprotech, Cat. 200-02), 10 ng/ml recombinant human interleukin 7 (Peprotech, Cat. 200-07), and 10 ng/ml recombinant human interleukin 15 (Peprotech, Cat. 200-15). T cells in TCPM (were seeded at 5×10\hat{ }4 cells per well in flat-bottom 96-well plates.

LNPs were generated as described in Example 1 at a molar ratio of 35 Lipid A/47.5 cholesterol/15 DSPC/2.5 PEG2k-DMG. Messenger RNA sequences are as described in Example 1. Prior to T cell treatments, different mixtures of LNPs were prepared in T cell treatment media (TC™): a version of TCGM containing 20 ug/mL rhApoE3 in the absence of interleukins 2, 7 or 15. The final concentration of each LNPs in every treatment group is shown in Table 9. LNP mixtures were incubated at 37° C. for 15 minutes and then added to 5×10\hat{ }4 T cells that were previously seeded in 96-well plates.

Next, Compound 1 and a repair template in the form of an adeno-associated virus (AAV) encoding a TCR (SEQ ID NO: 297) were diluted in TCTM and added to T cells at the final concentrations of 0.5 μM and 3×10\hat{ }5 genome copies/cell, respectively. T cells were incubated at 37° C. for 24 hours, at which time they were centrifuged at 500 g for 5 mi, resuspended in fresh TCGM and returned to the incubator. On day 4 post-treatment, T cells were sub-cultured at a 1:4 ratio (v/v) in TCGM. On day 7 post-treatment, flow cytometry was performed to assess the knockout efficiency of different surface receptors encoded by genes targeted by Sp base editor and the insertion efficiency of TCR in the TRAC locus by SpCas9 or Nme2 Cas9.

TABLE 9
Compositions of LNP mixtures in each treatment group.
The final concentration of each LNP is shown in μg/mL

| | Treatment groups | | | | | | |
|---|---|---|---|---|---|---|---|
| LNP | | AAV | SpBC22n | SpCas9 | Nme2 Cas9 | SpBC22n + | SpBC22n + |
| composition | Untreated | only | only | only | only | SpCas9 | Nme2 Cas9 |
| SpBC22n | — | — | 0.83 | — | — | 0.83 | 0.83 |
| mRNA | | | | | | | |
| UGI mRNA | — | — | 0.4175 | — | — | 0.4175 | 0.4175 |
| TRAC G023520 | — | — | 0.15 | — | — | 0.15 | 0.15 |
| SpBC22n guide | | | | | | | |
| TRBC1/2 | — | — | 0.29 | — | — | 0.29 | 0.29 |
| G023524 | | | | | | | |
| SpBC22n guide | | | | | | | |
| CIITA G023521 | — | — | 0.715 | — | — | 0.715 | 0.715 |
| SpBC22n guide | | | | | | | |
| HLA-A | — | — | 2.185 | — | — | 2.185 | 2.185 |
| G023524 | | | | | | | |
| SpBC22n guide | | | | | | | |
| SpCas9 mRNA | — | — | — | 0.4175 | — | 0.4175 | — |
| TRAC G013006 | — | — | — | 0.83 | — | 0.83 | — |
| Spy Cas9 guide | | | | | | | |
| Nme2 Cas9 | — | — | — | — | 0.4175 | — | 0.4175 |
| mRNA | | | | | | | |
| TRAC G021469 | — | — | — | — | 0.83 | — | 0.83 |
| Nme2 Cas9 | | | | | | | |
| guide | | | | | | | |
| HD1 TCR AAV | − | + | − | + | + | + | + |

### Example 3.3. Flow Cytometry

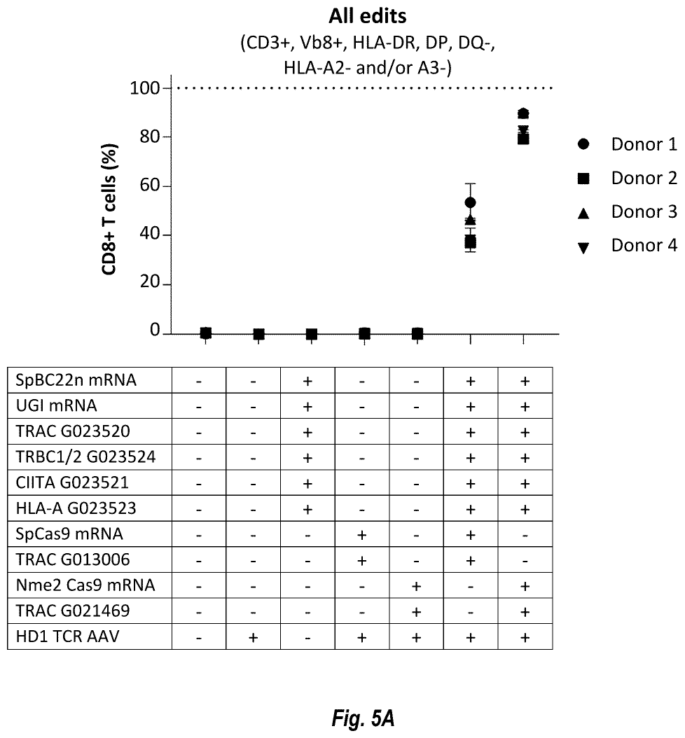

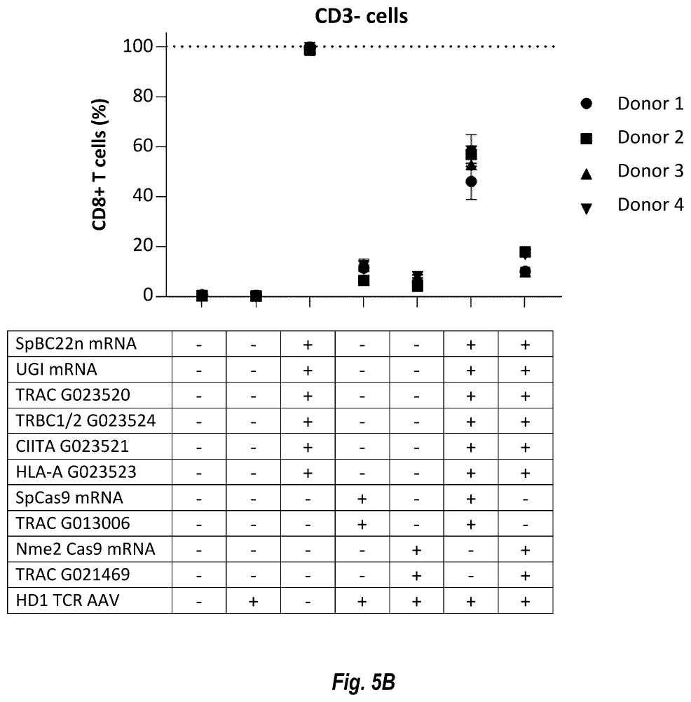

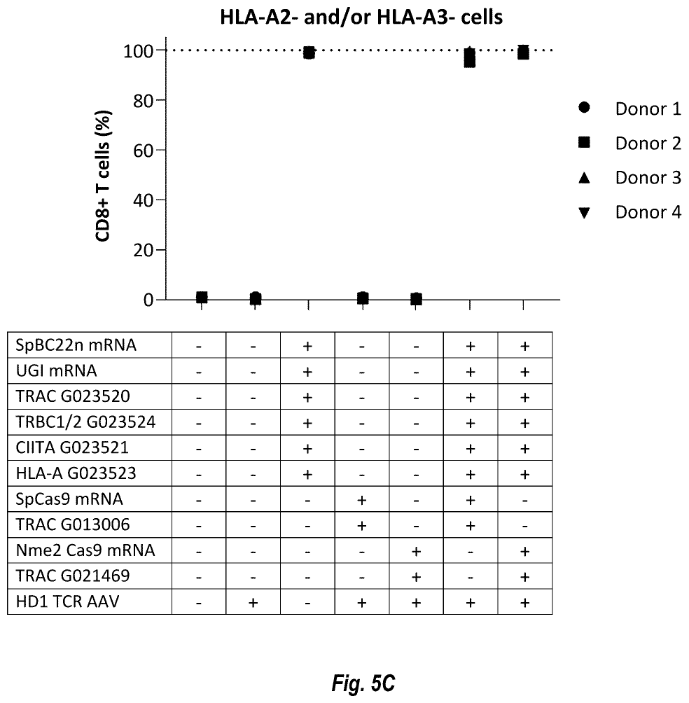

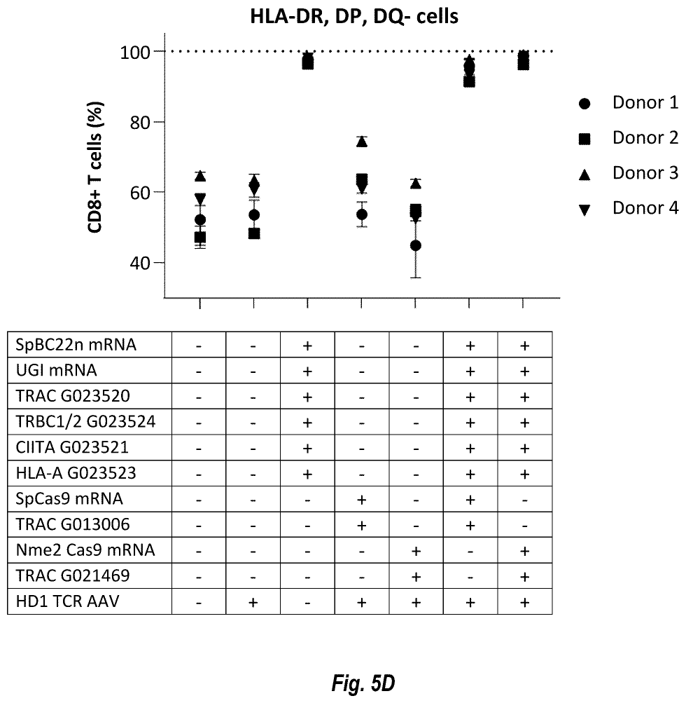

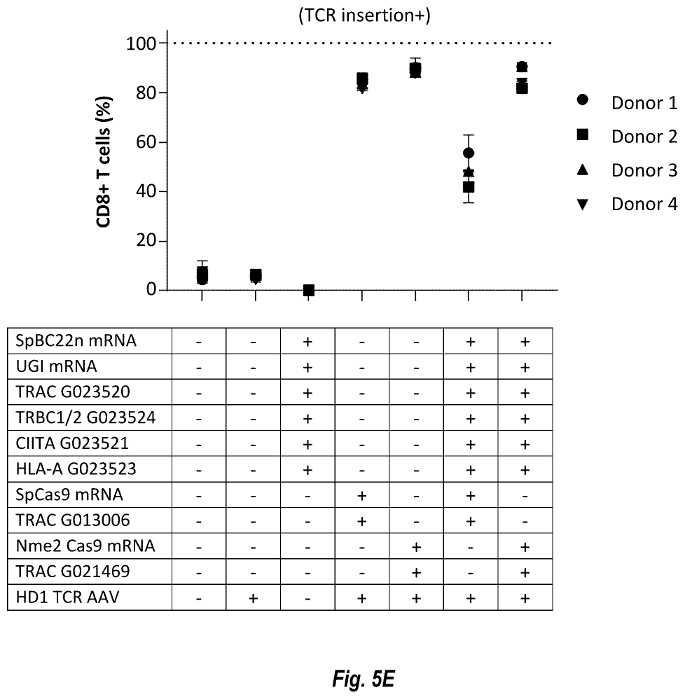

On day 7 post-LNP treatment, T cells were transferred to U-bottom 96-well plates and spun down for 5 minutes at 500 g. The supernatant was discarded, and cells were resuspended in FACS buffer containing Viakrome 808 (Beckman Coulter, Cat. C36628) (1:100), PC5.5 anti-human CD3 (Biolegend, Cat. 300430) (1:100), BV421 anti-human CD4 (Biolegend, Cat. 317434) (1:100), BV785 anti-human CD8 (Biolegend, Cat. 301046) (1:100), APC/Fire 750 anti-human HLA-DR, DP, DQ (Biolegend, Cat. 361712) (1:50), BV510 anti-human HLA-A2 (Biolegend, Cat. 343320) (1:100), FITC anti-human HLA-A3 (eBioscience Cat. 11-5754-42) (1:100), and PE anti-human TCR V08 (Biolegend, Cat. 348104) (1:100). T cells were stained for 30 minutes at 4° C. in the dark. T cells were washed once, resuspended in FACS buffer, and processed on a Cytoflex LX flow cytometer (Beckman Coulter). Flow cytometry data was processed on FlowJo version 10.8.1 (BD Biosciences). All T cells were gated on size, singularity, and viability and CD8+ expression. Percentages of CD8+ T cells negative for specific antigens and/or positive for TCR insertion are shown in Table 10 and FIGS. 5A-5E.

TABLE 10
Percentages of T cells negative for different antigens and/or positive for HD1 TCR insertion.

| | | Donor 1 | | | Donor 2 | | | Donor 3 | | | Donor 4 | | |
|---|---|---|---|---|---|---|---|---|---|---|---|---|---|
| Marker | Treatment group | Mean | SD | n | Mean | SD | n | Mean | SD | n | Mean | SD | n |
| CD3− | Untreated | 0.7 | 0.2 | 3 | 0.3 | 0.1 | 3 | 0.3 | 0.1 | 3 | 0.3 | 0.0 | 3 |
| | AAV only | 0.5 | 0.2 | 3 | 0.2 | 0.1 | 3 | 0.1 | 0.1 | 3 | 0.2 | 0.1 | 3 |
| | SpBC22n only | 99.7 | 0.0 | 3 | 98.5 | 0.2 | 3 | 99.4 | 0.1 | 3 | 99.4 | 0.1 | 2 |
| | SpCas9 only | 11.3 | 3.6 | 3 | 6.4 | 1.0 | 3 | 12.1 | 0.2 | 3 | 12.3 | 0.4 | 3 |
| | Nme2 Cas9 only | 5.8 | 3.1 | 3 | 4.1 | 0.1 | 3 | 7.0 | 0.6 | 3 | 7.9 | 0.6 | 3 |
| | SpBC22n + SpCas9 | 46.0 | 7.3 | 3 | 56.8 | 1.4 | 3 | 52.6 | 0.5 | 3 | 58.2 | 6.4 | 3 |
| | SpBC22n + Nme2 Cas9 | 10.1 | 1.5 | 3 | 17.9 | 0.8 | 3 | 9.8 | 0.3 | 3 | 16.8 | 1.0 | 3 |
| HLA-DR, | Untreated | 52.2 | 7.3 | 3 | 47.2 | 3.2 | 3 | 64.7 | 1.0 | 3 | 57.8 | 1.6 | 3 |
| DP, DQ− | AAV only | 53.6 | 4.1 | 3 | 48.3 | 1.3 | 3 | 63.4 | 1.7 | 3 | 60.6 | 2.1 | 3 |
| | SpBC22n only | 97.8 | 0.4 | 3 | 96.4 | 0.4 | 3 | 98.2 | 0.1 | 3 | 98.0 | 0.4 | 2 |
| | SpCas9 only | 53.7 | 3.5 | 3 | 63.7 | 0.7 | 3 | 74.5 | 1.3 | 3 | 60.9 | 1.2 | 3 |
| | Nme2 Cas9 only | 44.9 | 9.2 | 3 | 54.8 | 1.8 | 3 | 62.6 | 1.1 | 3 | 52.5 | 0.6 | 3 |
| | SpBC22n + SpCas9 | 95.7 | 2.3 | 3 | 91.4 | 0.3 | 3 | 97.7 | 0.1 | 3 | 93.3 | 2.7 | 3 |
| | SpBC22n + Nme2 Cas9 | 98.7 | 0.1 | 3 | 96.2 | 0.6 | 3 | 99.0 | 0.2 | 3 | 98.0 | 0.3 | 3 |
| Union of | Untreated | 1.2 | 0.2 | 3 | 0.8 | 0.2 | 3 | 1.2 | 0.4 | 3 | 1.1 | 0.2 | 3 |
| HLA-A2− | AAV only | 1.2 | 0.1 | 3 | 0.2 | 0.1 | 3 | 0.2 | 0.1 | 3 | 0.4 | 0.2 | 3 |
| and HLA- | SpBC22n only | 98.6 | 0.2 | 3 | 98.8 | 0.2 | 3 | 99.4 | 0.1 | 3 | 99.3 | 0.4 | 2 |
| A3− | SpCas9 only | 1.2 | 0.2 | 3 | 0.6 | 0.2 | 3 | 0.5 | 0.2 | 3 | 0.5 | 0.2 | 3 |
| | Nme2 Cas9 only | 0.7 | 0.2 | 3 | 0.1 | 0.0 | 3 | 0.2 | 0.0 | 3 | 0.4 | 0.3 | 3 |
| | SpBC22n + SpCas9 | 98.7 | 0.6 | 3 | 95.2 | 1.2 | 3 | 99.5 | 0.1 | 3 | 98.5 | 0.8 | 3 |
| | SpBC22n + Nme2 Cas9 | 99.9 | 0.1 | 3 | 98.5 | 0.2 | 3 | 99.9 | 0.0 | 3 | 99.9 | 0.0 | 3 |
| CD3+ Vb8+ | Untreated | 4.4 | 0.5 | 3 | 6.6 | 1.1 | 3 | 7.3 | 0.6 | 3 | 7.5 | 4.5 | 3 |
| | AAV only | 5.8 | 1.5 | 3 | 6.5 | 0.3 | 3 | 6.2 | 0.6 | 3 | 4.4 | 1.0 | 3 |
| | SpBC22n only | 0.0 | 0.0 | 3 | 0.1 | 0.0 | 3 | 0.0 | 0.0 | 3 | 0.0 | 0.0 | 2 |
| | SpCas9 only | 84.2 | 3.3 | 3 | 85.8 | 0.6 | 3 | 83.9 | 0.4 | 3 | 81.6 | 0.2 | 3 |
| | Nme2 Cas9 only | 90.0 | 3.9 | 3 | 89.8 | 1.2 | 3 | 88.3 | 1.0 | 3 | 87.8 | 1.1 | 3 |
| | SpBC22n + SpCas9 | 55.6 | 7.2 | 3 | 41.9 | 1.1 | 3 | 48.2 | 0.7 | 3 | 41.8 | 6.3 | 3 |
| | SpBC22n + Nme2 Cas9 | 90.5 | 1.5 | 3 | 81.8 | 0.6 | 3 | 90.5 | 0.2 | 3 | 83.7 | 0.9 | 3 |
| CD3+, | Untreated | 0.1 | 0.2 | 3 | 0.5 | 0.1 | 3 | 0.8 | 0.4 | 3 | 0.6 | 0.2 | 3 |
| Vb8+, | AAV only | 0.1 | 0.1 | 3 | 0.0 | 0.0 | 3 | 0.0 | 0.0 | 3 | 0.0 | 0.0 | 3 |
| HLA-DR, | SpBC22n only | 0.0 | 0.0 | 3 | 0.0 | 0.0 | 3 | 0.0 | 0.0 | 3 | 0.0 | 0.0 | 3 |
| DP, DQ−, | SpCas9 only | 0.6 | 0.2 | 3 | 0.1 | 0.0 | 3 | 0.2 | 0.1 | 3 | 0.2 | 0.1 | 3 |
| [HLA-A2−/ | Nme2 Cas9 only | 0.5 | 0.1 | 3 | 0.1 | 0.0 | 3 | 0.1 | 0.0 | 3 | 0.2 | 0.1 | 3 |
| HLA-A | SpBC22n + SpCas9 | 53.5 | 7.6 | 3 | 37.1 | 0.4 | 3 | 46.7 | 0.4 | 3 | 38.2 | 4.8 | 3 |
| A3−] | SpBC22n + Nme2 Cas9 | 89.6 | 1.3 | 3 | 79.2 | 0.6 | 3 | 89.8 | 0.2 | 3 | 82.5 | 0.6 | 3 |

### Example 4. Simultaneous Multi-Edit with Nme2Cas9 and SpBC22n in Primary Mouse Hepatocytes

Primary mouse hepatocytes (PMH) were edited simultaneously at the albumin locus using NmeCas9 and at the TTR locus using SpCas9 or SpBC22n base editor. PMH (Gibco, Lot MC931) were thawed and resuspended in hepatocyte thawing medium with plating supplements (William's E Medium (Gibco, Cat. A12176-01)) with dexamethasone+cocktail supplement (Gibco, Cat. A15563, Lot 2019842) and Plating Supplements with FBS content (Gibco, Cat. A13450, Lot 1970698) followed by centrifugation. The supernatant was discarded, and the pelleted cells resuspended in hepatocyte plating medium plus supplement pack (Invitrogen, Cat. A1217601 and Gibco, Cat. CM3000). Cells were counted and plated on Bio-coat collagen I coated 96-well plates (ThermoFisher, Cat. 877272) at a concentration of 20,000 cells/well. Plated cells were allowed to settle and adhere for 4-6 hours in a tissue culture incubator at 37° C. and 5% CO2 atmosphere. After incubation cells were checked for monolayer formation and were washed once with hepatocyte maintenance medium (Invitrogen, Cat. A1217601 and Gibco, Cat. CM4000). Each condition was tested with technical duplicate samples. Cellartis PowerHEP Medium (Takara Bio, Y20020) was added for a total volume of 100 uL for each plate.

LNPs were generally prepared as described in Example 1 with either a single RNA species as cargo, or a co-formulation of gRNA and mRNA. The LNPs were prepared with a molar ratio of 50 Lipid A:38 cholesterol:9 The LNPs were prepared with a molar ratio of 50 Lipid A:38 cholesterol:9 DSPC: 3 PEG2k-DMG. Messenger RNA sequences are as described in Example 1. The LNPs were formulated with a lipid amine to RNA phosphate (N:P) molar ratio of about 6. For Nme2Cas9 mRNA and albumin guide coformulation, LNPs were prepared with a ratio of 2:1 by weight of gRNA to editor mRNA cargo. For SpyCas9 and SpyCas9 base editor mRNA and TTR guide single formulations, LNPs were prepared with a single RNA species as cargo and LNPs were mixed at a ratio of 1:2 by weight of gRNA cargo to editor mRNA cargo before treatment.

Cells were treated with LNPs at the dosages indicated in Table 11. Dilution series indicates an 8-point, three-fold dilution series of doses were used starting at a high concentration of 300 ng combined guide and editor mRNA. Each sample was treated with an additional 5 ng LNP containing UGI mRNA. Insertion efficiency was tested in cells treated with an AAV vector encoding a NanoLuc template (SEQ ID No: 304) at a multiplicity of infection (MOI) of 5E5, and quantified via expression of NanoLuc template using the Nano-Glo Luciferase assay. Editing efficiency was tested in cells that did not receive AAV treatment. The total volume of all components delivered was 100 uL/well.

TABLE 11

| Group | Treatment | mRNA | gRNA | Dosage |
|---|---|---|---|---|
| Nme2Cas9 | LNP | Nme2Cas9 | G023805 | Dilution series |
| insertion only | | | (albumin) | |
| SpCas9 | LNP mix | SpCas9 | G025427 | Dilution Series |
| insertion only | | | (TTR) | |
| Spy Base Edit | LNP mix | Sp BE | G025427 | Dilution Series |
| only | | | (TTR) | |
| SpCas9 + | LNP | Nme2Cas9 | G023805 | 100 ng combined |
| Fixed A1AT | | | (albumin) | RNA |
| Insertion | LNP mix | SpCas9 | G025427 | Dilution Series |
| | | | (TTR) | |
| Spy Base edit + | LNP | Nme2Cas9 | G023805 | 100 ng combined |
| Fixed A1AT | | | (albumin) | RNA |
| Insertion | LNP mix | Sp BE | G025427 | Dilution Series |
| | | | (TTR) | |
| Insertion + | LNP | Nme2Cas9 | G023805 | Dilution Series |
| Fixed SpCas9 | | | (albumin) | |
| | LNP mix | SpCas9 | G025427 | 100 ng combined |
| | | | (TTR) | RNA |
| Insertion + | LNP | Nme2Cas9 | G023805 | Dilution Series |
| Fixed Base | | | (albumin) | |
| Edit | LNP mix | Sp BE | G025427 | 100 ng combined |
| | | | (TTR) | RNA |

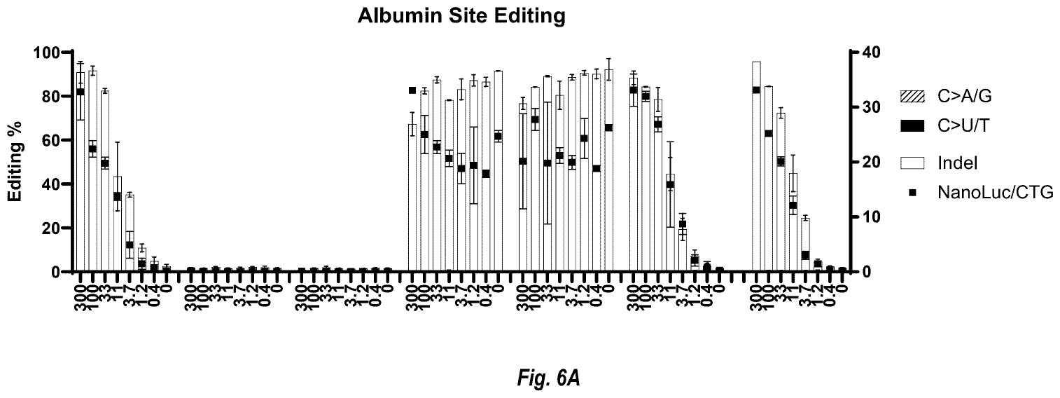

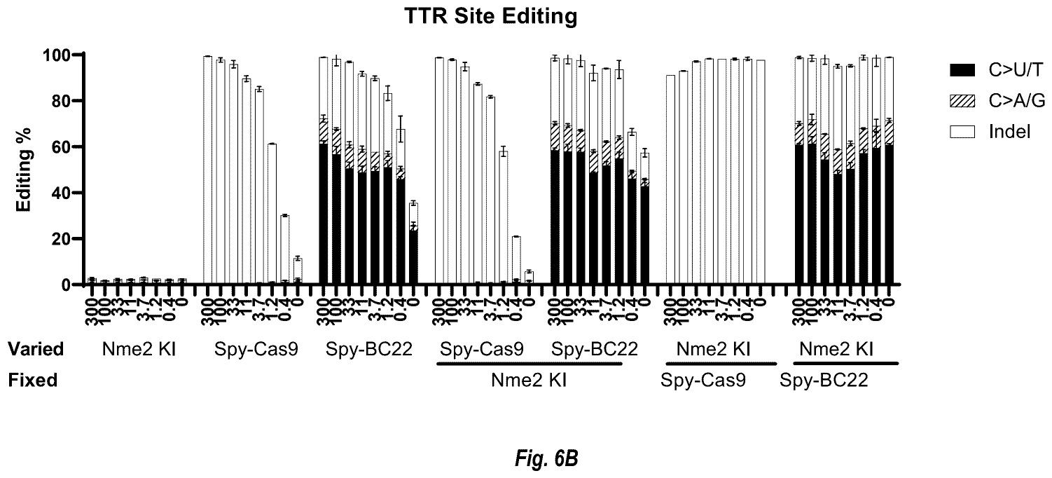

Seventy-two hours post treatment, transfection plates for editing readout were subjected to lysis, PCR amplification of the TTR and Albumin locus, and subsequent NGS analysis, as described in Example 1. All experiments were performed in biological duplicates. Table 12 and FIG. 6B show mean percent editing at the TTR locus.

TABLE 12
Mean percent editing at the TTR locus following
treatment with SpCas9 or Sp base editor.

| | Editor + | % C to T | | % C to A/G | | % Indel | |
|---|---|---|---|---|---|---|---|
| Treatment | gRNA (ng) | Mean | SD | Mean | SD | Mean | SD |
| Insertion | 300.0 | 0.78 | 0.20 | 1.32 | 0.39 | 0.73 | 0.33 |
| only | 100.0 | 0.54 | 0.16 | 0.91 | 0.11 | 0.36 | 0.09 |
| | 33.0 | 0.73 | 0.24 | 1.02 | 0.01 | 0.66 | 0.28 |
| | 11.0 | 0.90 | 0.05 | 1.04 | 0.18 | 0.50 | 0.03 |
| | 3.7 | 0.87 | 0.17 | 1.22 | 0.08 | 0.98 | 0.18 |
| | 1.2 | 0.98 | 0.57 | 0.92 | 0.10 | 0.66 | 0.00 |
| | 0.4 | 0.83 | 0.27 | 1.00 | 0.03 | 0.50 | 0.04 |
| | 0.0 | 0.80 | 0.19 | 1.20 | 0.34 | 0.53 | 0.06 |
| SpCas9 | 300.0 | 0.02 | 0.01 | 0.14 | 0.00 | 99.24 | 0.08 |
| | 100.0 | 0.02 | 0.01 | 0.06 | 0.02 | 97.71 | 0.97 |
| | 33.0 | 0.13 | 0.14 | 0.11 | 0.01 | 95.67 | 1.62 |
| | 11.0 | 0.31 | 0.14 | 0.28 | 0.04 | 89.06 | 1.26 |
| | 3.7 | 0.53 | 0.22 | 0.27 | 0.10 | 84.35 | 1.10 |
| | 1.2 | 0.70 | 0.23 | 0.46 | 0.02 | 60.17 | 0.15 |
| | 0.4 | 1.14 | 0.62 | 0.70 | 0.01 | 28.28 | 0.45 |
| | 0.0 | 1.09 | 0.12 | 1.27 | 0.54 | 9.10 | 0.90 |
| Base Edit | 300.0 | 61.27 | 1.27 | 11.00 | 1.52 | 26.68 | 0.06 |
| only | 100.0 | 56.74 | 3.40 | 10.98 | 0.58 | 30.52 | 3.01 |
| | 33.0 | 50.56 | 2.71 | 10.34 | 1.34 | 36.02 | 0.31 |
| | 11.0 | 48.79 | 2.79 | 10.21 | 1.28 | 32.73 | 1.03 |
| | 3.7 | 49.46 | 1.84 | 8.27 | 0.01 | 32.07 | 0.98 |
| | 1.2 | 51.11 | 2.84 | 5.88 | 1.07 | 26.28 | 3.16 |
| | 0.4 | 45.89 | 1.17 | 4.56 | 0.98 | 17.27 | 5.70 |
| | 0.0 | 23.53 | 3.66 | 2.10 | 0.13 | 9.93 | 0.98 |
| SpCas9 + | 300.0 | 0.02 | 0.01 | 0.14 | 0.17 | 98.60 | 0.13 |
| Fixed | 100.0 | 0.06 | 0.04 | 0.21 | 0.21 | 97.61 | 0.33 |
| Insertion | 33.0 | 0.10 | 0.12 | 0.09 | 0.01 | 94.67 | 1.82 |
| | 11.0 | 0.36 | 0.26 | 0.44 | 0.35 | 86.53 | 0.54 |
| | 3.7 | 0.46 | 0.08 | 0.24 | 0.07 | 81.06 | 0.50 |
| | 1.2 | 0.65 | 0.14 | 0.60 | 0.11 | 56.78 | 2.21 |
| | 0.4 | 1.25 | 0.36 | 1.03 | 0.18 | 18.66 | 0.18 |
| | 0.0 | 0.71 | 0.00 | 0.98 | 0.21 | 4.06 | 0.59 |
| Base edit + | 300.0 | 58.44 | 1.05 | 11.80 | 0.70 | 28.44 | 1.27 |
| Fixed | 100.0 | 57.96 | 3.20 | 11.30 | 0.78 | 29.11 | 2.28 |
| Insertion | 33.0 | 57.80 | 1.62 | 9.39 | 0.35 | 30.47 | 2.81 |
| | 11.0 | 48.69 | 0.37 | 9.39 | 0.64 | 34.05 | 3.43 |
| | 3.7 | 51.81 | 1.75 | 10.39 | 0.33 | 31.84 | 0.18 |
| | 1.2 | 54.91 | 2.76 | 9.05 | 0.78 | 29.69 | 3.90 |
| | 0.4 | 46.13 | 2.51 | 3.35 | 0.29 | 16.98 | 1.49 |
| | 0.0 | 42.78 | 1.66 | 3.08 | 0.32 | 11.53 | 1.87 |
| Insertion + | 300.0 | 0.07 | 0.04 | 0.17 | 0.01 | 90.88 | 0.01 |
| Fixed | 100.0 | 0.05 | 0.00 | 0.11 | 0.04 | 92.86 | 0.14 |
| SpCas9 | 33.0 | 0.02 | 0.01 | 0.14 | 0.00 | 96.98 | 0.27 |
| | 11.0 | 0.03 | 0.00 | 0.08 | 0.02 | 98.17 | 0.10 |
| | 3.7 | 0.04 | 0.01 | 0.10 | 0.01 | 97.99 | 0.02 |
| | 1.2 | 0.06 | 0.02 | 0.11 | 0.02 | 98.02 | 0.36 |
| | 0.4 | 0.03 | 0.03 | 0.10 | 0.03 | 98.12 | 0.67 |
| | 0.0 | 0.04 | 0.00 | 0.12 | 0.06 | 97.56 | 0.01 |
| Insertion + | 300.0 | 60.82 | 0.62 | 9.31 | 0.81 | 28.69 | 0.45 |
| Fixed Base | 100.0 | 61.26 | 3.13 | 10.58 | 2.28 | 26.73 | 1.34 |
| Edit | 33.0 | 54.48 | 2.86 | 11.08 | 0.12 | 32.86 | 2.55 |
| | 11.0 | 48.15 | 1.58 | 10.69 | 0.21 | 36.16 | 0.78 |
| | 3.7 | 50.37 | 2.72 | 11.11 | 1.00 | 33.73 | 0.52 |
| | 1.2 | 57.19 | 1.63 | 10.77 | 0.29 | 30.94 | 1.10 |
| | 0.4 | 59.47 | 7.16 | 9.58 | 2.93 | 29.58 | 3.70 |
| | 0.0 | 60.91 | 0.45 | 10.56 | 0.89 | 27.46 | 0.06 |

For transfection plates for insertion readout, 50 uL of media from each well was added to equal amounts of prepared Nano-Glo Luciferase assay reagent (Promega N1110) following manufacturer's instructions to quantify NanoLuc signal and read in a Biotek Neo2 plate reader. The remaining media was aspirated from the insertion readout plates, and 100 uL of prepared Cell TiterGlo reagent (Promega G9241) to quantify cell viability, and read in a Biotek Neo2 plate reader. NanoLuc Signal was normalized via cell viability by dividing the NanoLuc signal by the cell viability signal. Table 13 and FIG. 6A show mean percent indels at the albumin locus as assayed in the no-AAV samples. Table 13 and FIG. 6A also show mean fluorescence normalized by cell viability as a read out of relative insertion efficiency across the population.

TABLE 13
Insertion of NanoLuc at the albumin locus expressed
as luminescence (RLU) normalized to cell viability

| | Editor + | | | | Insertion | | |
|---|---|---|---|---|---|---|---|
| | gRNA | % Indel | | | (RLU/cell viability) | | |
| Treatment | (ng) | Mean | SD | n | Mean | SD | n |
| Insertion | 300 | 90.66 | 4.91 | 2 | 32.80 | 5.15 | 2 |
| only | 100 | 91.46 | 2.06 | 2 | 22.40 | 1.51 | 2 |
| | 33 | 82.17 | 1.06 | 2 | 19.80 | 1.10 | 2 |
| | 11 | 43.08 | 15.62 | 2 | 13.73 | 0.70 | 2 |
| | 3.7 | 34.69 | 1.10 | 2 | 4.91 | 2.43 | 2 |
| | 1.2 | 10.32 | 1.81 | 2 | 1.47 | 1.01 | 2 |
| | 0.4 | 4.26 | 1.78 | 2 | 0.74 | 0.59 | 2 |
| | 0 | 1.91 | 0.88 | 2 | 0.20 | 0.00 | 2 |
| SpCas9 | 300 | 1.18 | 0.25 | 2 | 0.22 | 0.00 | 2 |
| | 100 | 0.93 | 0.18 | 2 | 0.10 | 0.04 | 2 |
| | 33 | 1.55 | 0.26 | 2 | 0.08 | 0.01 | 2 |
| | 11 | 0.93 | 0.31 | 2 | 0.07 | 0.01 | 2 |
| | 3.7 | 1.07 | 0.50 | 2 | 0.10 | 0.00 | 2 |
| | 1.2 | 1.40 | 0.34 | 2 | 0.09 | 0.01 | 2 |
| | 0.4 | 1.30 | 0.64 | 2 | 0.06 | 0.02 | 2 |
| | 0 | 0.94 | 0.13 | 2 | 0.06 | 0.01 | 2 |
| Base Edit | 300 | 0.40 | 0.30 | 2 | 0.12 | 0.03 | 2 |
| only | 100 | 0.95 | 0.17 | 2 | 0.09 | 0.01 | 2 |
| | 33 | 1.03 | 0.59 | 2 | 0.07 | 0.01 | 2 |
| | 11 | 0.73 | 0.28 | 2 | 0.05 | 0.02 | 2 |
| | 3.7 | 0.80 | 0.00 | 2 | 0.06 | 0.02 | 2 |
| | 1.2 | 0.70 | 0.10 | 2 | 0.07 | 0.02 | 2 |
| | 0.4 | 1.03 | 0.14 | 2 | 0.07 | 0.02 | 2 |
| | 0 | 0.88 | 0.18 | 2 | 0.07 | 0.01 | 2 |
| SpCas9 + | 300 | 67.04 | 5.35 | 2 | 33.10 | 0.00 | 1 |
| Fixed | 100 | 82.16 | 1.43 | 2 | 25.01 | 3.47 | 2 |
| Insertion | 33 | 87.32 | 1.43 | 2 | 22.74 | 1.18 | 2 |
| | 11 | 77.77 | 0.17 | 2 | 20.69 | 1.50 | 2 |
| | 3.7 | 82.93 | 4.74 | 2 | 18.82 | 2.77 | 2 |
| | 1.2 | 86.97 | 2.58 | 2 | 19.41 | 6.97 | 2 |
| | 0.4 | 86.17 | 2.09 | 2 | 17.86 | 0.65 | 2 |
| | 0 | 91.25 | 0.08 | 2 | 24.70 | 1.05 | 2 |
| Base edit + | 300 | 76.37 | 2.74 | 2 | 20.16 | 8.66 | 2 |
| Fixed | 100 | 83.83 | 0.07 | 2 | 27.76 | 2.00 | 2 |
| Insertion | 33 | 88.91 | 0.28 | 2 | 19.82 | 11.09 | 2 |
| | 11 | 80.21 | 6.44 | 2 | 21.19 | 1.43 | 2 |
| | 3.7 | 88.42 | 1.23 | 2 | 19.94 | 1.25 | 2 |
| | 1.2 | 90.44 | 1.06 | 2 | 24.31 | 3.64 | 2 |
| | 0.4 | 89.96 | 2.22 | 2 | 18.80 | 0.53 | 2 |
| | 0 | 92.06 | 4.89 | 2 | 26.27 | 0.57 | 2 |
| Insertion + | 300 | 88.20 | 3.05 | 2 | 33.10 | 2.95 | 2 |
| Fixed | 100 | 84.11 | 0.16 | 2 | 31.97 | 0.94 | 2 |
| SpCas9 | 33 | 78.36 | 5.41 | 2 | 26.88 | 1.38 | 2 |
| | 11 | 44.14 | 7.61 | 2 | 15.93 | 7.78 | 2 |
| | 3.7 | 18.85 | 5.05 | 2 | 8.73 | 1.91 | 2 |
| | 1.2 | 6.70 | 2.71 | 2 | 2.09 | 1.04 | 2 |
| | 0.4 | 2.15 | 1.27 | 2 | 0.82 | 1.06 | 2 |
| | 0 | 1.11 | 0.06 | 2 | 0.30 | 0.08 | 2 |
| Insertion + | 300 | 95.57 | 0.00 | 2 | 33.11 | 0.00 | 1 |
| Fixed Base | 100 | 84.00 | 0.10 | 2 | 25.21 | 0.51 | 2 |
| Edit | 33 | 71.87 | 2.41 | 2 | 20.12 | 0.83 | 2 |
| | 11 | 44.47 | 8.35 | 2 | 12.13 | 1.68 | 2 |
| | 3.7 | 24.06 | 1.23 | 2 | 3.01 | 0.75 | 2 |
| | 1.2 | 4.01 | 1.13 | 2 | 1.45 | 0.53 | 2 |
| | 0.4 | 1.74 | 0.38 | 2 | 0.38 | 0.01 | 2 |
| | 0 | 1.16 | 0.22 | 2 | 0.17 | 0.01 | 2 |

### Example 5. In Vivo Orthogonal Editing and Insertion

The sgRNAs tested in Example 4 were evaluated in vivo to assess the efficiency of simultaneous editing at the TTR locus using NmeCas9 with SpCas9 or Sp base editor and insertion of SERPINA1 encoding A1AT protein into the albumin locus in the mouse model following LNP delivery.

The LNPs used in this experiment were formulated and prepared as described in Example 4 and contained a single RNA species as cargo, or a co-formulation of gRNA and mRNA. Messenger RNAs used are those described in Example 1. The LNPs formulated were dosed with the sgRNA and mRNA as shown in Table 14. LNPs were delivered with 100 uL of AAV (SEQ ID NO: 298) diluted in 1× phosphate buffered saline+0.0001% PF-68.

TABLE 14
LNP formulations delivered in vivo

| | | Cargo ratio | | | Dose |
|---|---|---|---|---|---|
| Group | LNP Cargo | (sgRNA:mRNA) | Guide ID | mRNA | (mg/kg) |
| 1 | TSS | | | | |
| 2 | co-formulation | 2:1 | G23805 | Nme2 Cas9 | 0.50 |
| 3 | Single cargo | | G25427 | | 0.10 |
| | | | | Sp BC22n BE | 0.20 |
| | | | | UGI | 0.10 |
| 4 | co-formulation | 2:1 | G23805 | Nme2 Ca9 | 0.50 |
| | Single cargo | | G25427 | | 0.10 |
| | | | | Sp BC22n BE | 0.20 |
| | | | | UGI | 0.10 |
| 5 | co-formulation | 1:2 | G25427 | | 0.10 |
| | | | | Sp Cas9 | 0.20 |
| 6 | co-formulation | 1:2 | G23805 | Nme2 Cas9 | 0.50 |
| | Single cargo | | G25427 | | 0.10 |
| | | | | Sp Cas9 | 0.20 |
| | | | | UGI | 0.10 |

C5BL/6 male mice, ranging 6-10 weeks of age were used in each study involving mice (n=5 per group, except TSS control n=4). LNPs and AAVs were administered intravenously via tail vein injection at the doses shown in Table 14. Animals were periodically observed for adverse effects for at least 24 hours post-dose. Interim in-life tail bleeds were performed at 1, 2, and 4 weeks post dose to quantify hA1AT protein in mouse serum by ELISA analysis. Briefly, the hA1AT serum levels were determined using an Aviva Alpha 1-antiTrypsin ELISA kit, Human (Catalog #OKIA00048) according to the manufacturer's protocol. Mouse serum was diluted to a final dilution of 10,000-fold with 1× assay diluent. This was done by carrying out two sequential 50-fold dilutions resulting in a 2500-fold dilution. A final 4-fold dilution step was carried out for a total sample dilution of 10,000-fold. Both standard curve dilutions (100 μL each) and diluted serum samples were added to each well of the ELISA plate pre-coated with capture antibody. The plate was incubated at room temperature for 30 minutes before washing. Enzyme-antibody conjugate (100 μL per well) was added for a 20-minute incubation. Unbound antibody conjugate was removed and the plate was washed again before the addition of the chromogenic substrate solution. The plate was incubated for 10 minutes before adding 100 μL of the stop solution, e.g., sulfuric acid (approximately 0.3 M). The plate was read on a SpectraMax M5 or Clariostar plate reader at an absorbance of 450 nm. Serum TTR levels were calculated by SoftMax Pro software ver. 6.4.2 or Mars software ver. 3.31 using a four-parameter logistic curve fit off the standard curve. Final serum values were adjusted for the assay dilution. Percent protein knockdown (% KD) values were determined relative to controls, which generally were animals sham-treated with vehicle (TSS) unless otherwise indicated.

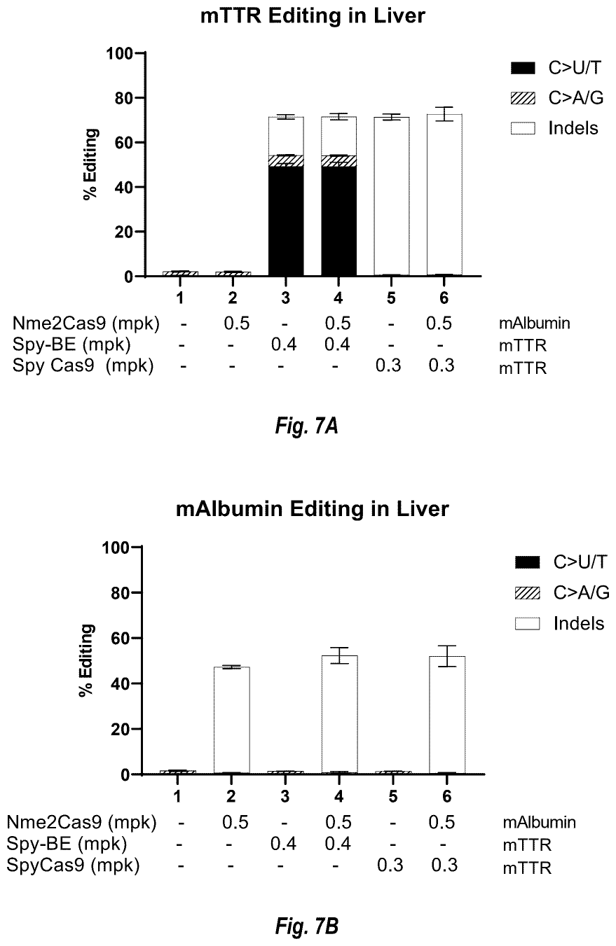

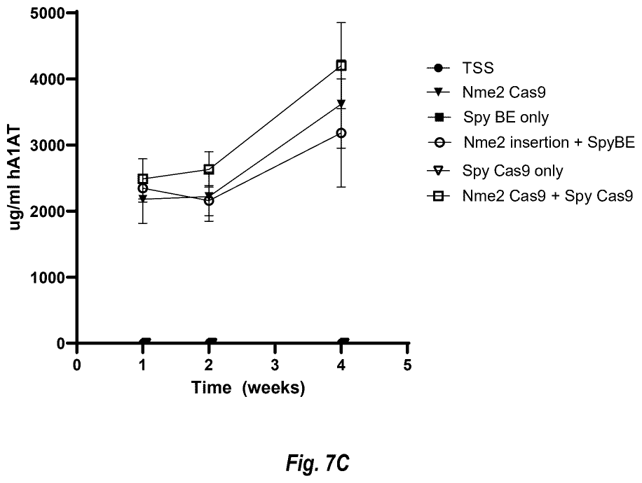

Animals were euthanized five weeks post-injection by cardiac puncture under isoflurane anesthesia; liver tissue were collected for downstream analysis. Liver punches weighing between 5 and 15 mg were collected for isolation of genomic DNA and total RNA. Genomic DNA was extracted using a DNA isolation kit (ZymoResearch, D3010) and samples were analyzed with NGS sequencing as described in Example 1. The TTR editing efficiency for LNPs containing the indicated gRNAs are shown in Table 15 and FIG. 7A. Albumin editing efficiency is shown in Table 16 and FIG. 71B. Serum protein levels of hA1AT are shown in Table 17 and FIG. 7C.

TABLE 15
Mean percent TTR editing in mouse liver.

| | C-to-T % | | C-to-A/G % | | Indel % | | |
|---|---|---|---|---|---|---|---|
| | Mean | SD | Mean | SD | Mean | SD | n |
| TSS | 0.12 | 0.25 | 1.97 | 0.16 | 0.10 | 0.10 | 5 |
| Nme2Cas9 insertion | 24.67 | 0.05 | 1.93 | 0.25 | 0.09 | 0.12 | 5 |
| only | | | | | | | |
| Spy BE only | 49.24 | 1.16 | 4.90 | 0.22 | 17.29 | 0.88 | 5 |
| Nme2Cas9 insertion + | 21.86 | 1.68 | 4.94 | 0.22 | 17.41 | 1.23 | 4 |
| Spy BE | | | | | | | |
| SpyCas9 only | 0.03 | 0.01 | 0.62 | 0.08 | 70.75 | 1.23 | 5 |
| Nme2Cas9 insertion + | 0.05 | 0.03 | 0.67 | 0.15 | 71.99 | 2.77 | 5 |
| SpyCas9 | | | | | | | |

TABLE 16
Mean percent albumin editing in mouse liver.

| | C-to-T % | | | C-to-A/G % | | | Indel % | | |
|---|---|---|---|---|---|---|---|---|---|
| | Mean | SD | n | Mean | SD | n | Mean | SD | n |
| TSS | 0.05 | 0.04 | 5 | 1.49 | 0.22 | 5 | 0.13 | 0.21 | 5 |
| Nme2Cas9 insertion | 0.02 | 0.01 | 5 | 0.65 | 0.08 | 5 | 46.58 | 0.60 | 5 |
| only | | | | | | | | | |
| Spy BE only | 0.06 | 0.02 | 5 | 1.29 | 0.07 | 5 | 0.07 | 0.04 | 5 |
| Nme2Cas9 insertion + | 0.03 | 0.03 | 5 | 0.85 | 0.37 | 5 | 51.42 | 3.02 | 4 |
| Spy BE | | | | | | | | | |
| SpyCas9 only | 0.07 | 0.02 | 5 | 1.30 | 0.08 | 5 | 0.03 | 0.02 | 5 |
| Nme2Cas9 insertion + | 0.01 | 0.01 | 3 | 0.57 | 0.16 | 3 | 51.44 | 3.75 | 3 |
| SpyCas9 | | | | | | | | | |

TABLE 17
Serum protein levels of hA1AT.

| | Weeks | Mean | SD | n |
|---|---|---|---|---|
| TSS | 1 | 0 | 0 | 5 |
| | 2 | 0 | 0 | 5 |
| | 4 | 0 | 0 | 5 |
| Nme2Cas9 insertion | 1 | 2179.22 | 326.30 | 5 |
| only | 2 | 2220.62 | 333.81 | 5 |
| | 4 | 3619.96 | 598.05 | 5 |
| Spy BE only | 1 | 0 | 0 | 5 |
| | 2 | 0 | 0 | 5 |
| | 4 | 0 | 0 | 5 |
| Nme2Cas9 insertion + | 1 | 2347.69 | 188.48 | 5 |
| Spy BE | 2 | 2158.60 | 196.80 | 4 |
| | 4 | 3183.90 | 709.25 | 4 |
| SpyCas9 only | 1 | 0 | 0 | 5 |
| | 2 | 0 | 0 | 5 |
| | 4 | 0 | 0 | 5 |
| Nme2Cas9 insertion + | 1 | 2486.46 | 271.82 | 5 |
| SpyCas9 | 2 | 2629.86 | 238.45 | 5 |
| | 4 | 4201.42 | 582.27 | 5 |

### Example 6. Screening of Insertion Guide RNAs with Gapped AAV Templates and SpyCas9

AAVS1 guide RNAs were designed and screened to identify insertion SpyCas9 guides using a series of gapped AAV GFP templates (A, B, C, D, OG) each designed for a subset of the guide RNAs. Insertion efficacy in T cells was examined by assessing GFP expression by flow cytometry. The percentage of T cells positive for green fluorescent protein (“% GFP+”) was assayed by flow cytometry, following AAVS1 editing by mRNA and AAV delivery.

### Example 6.1. T Cell Preparation

Healthy human donor apheresis was obtained commercially (Hemacare) from two donors (#110042863 and #110040377) and cells were washed and resuspended in MACS Buffer containing 2 mM EDTA and 0.5% Fetal Bovine Serum (FBS) in PBS. Cells were washed twice by centrifugation followed by CD3 negative selection using Easy Sep Human T Cell Isolation Kit (Stemcell, Cat. 100-0695) and separated using Easy Sep Magnets (Stemcell, Cat.18000). T cells were aliquoted and cryopreserved for future use in Cryostor® CS10 (StemCell Technologies Cat. 07930).

### Example 6.2. RNP Electroporation of T Cells

AAVS1 guide RNAs were assessed for insertion efficacy in T cells by assessing GFP expression by flow cytometry. The percentage of T cells positive for green fluorescent protein (“% GFP+”) was assayed by flow cytometry, following AAVS1 editing by mRNA and AAV delivery.

AAVS1 targeting sgRNAs corresponding to the flanking homology regions of Gap Templates A, B, C, D, & OG (SEQ ID NOs: 299-303) were removed from their storage plates and denatured for 2 minutes at 95° C. before cooling at room temperature for 10 minutes. RNP mixture of 20 μM sgRNA and 10 μM Cas9-NLS protein (SEQ ID NO: 296) was prepared and incubated at 25° C. for 10 minutes. 2.5 μL of RNP mixture was combined with 250,000 cells in 20 μL P3 electroporation Buffer (Lonza). 22 μL of RNP/cell mix was transferred to the corresponding wells of a Lonza shuttle 96-well electroporation plate. Cells were electroporated in duplicate with the manufacturer's pulse code. T cell media described above without any cytokines was added to the cells immediately post electroporation. T cells were rested for 10 minutes at 37° C. Gap Template AAVs were prepared in 48 well plates (Corning, Cat.353078)) with T cell media described above containing 2× cytokines, 400 U/mL recombinant human interleukin-2 (Peprotech, Cat. 200-02), 10 ng/ml recombinant human interleukin 7 (Peprotech, Cat. 200-07), and 10 ng/ml recombinant human interleukin 15 (Peprotech, Cat. 200-15) cytokines. The multiplicity of infection (MOI) of AAVs was 3×10\hat{ }⁵. AAVs were added to the T cells within 15 minutes post electroporation and incubated for 48 hours at 37° C. AAV-6 vectors encoded AAVS1 Template A, B, C, D or OG. Two days post-electroporation, cells were split 1:2 in 48 well plates replenished with T cell media with 1× cytokines as described above. sgRNAs were tested each with AAV constructs and gapped template.

### Example 6.3. Flow Cytometry

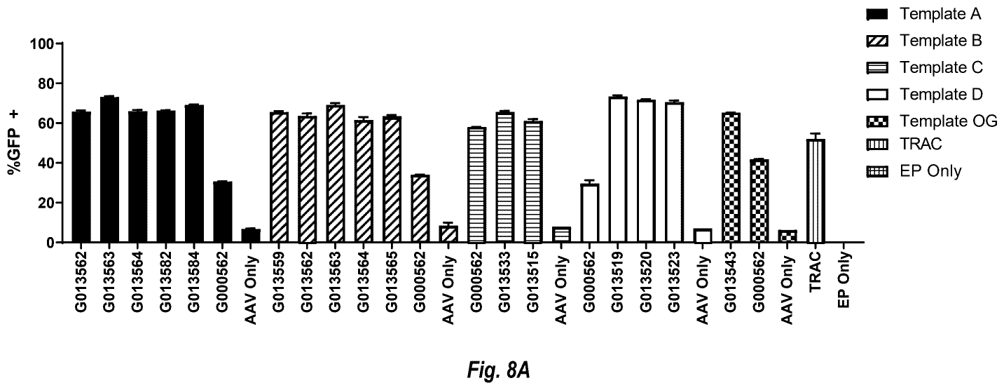

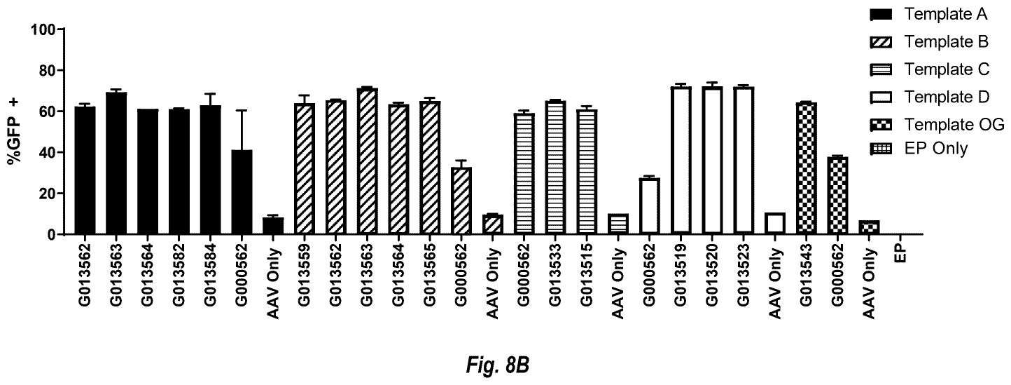

On day 7 post-editing, cells were phenotyped by flow cytometry to determine GFP expression to confirm integration of AAV at their GAP template site. Briefly, T cells were washed in FACS buffer containing 2 mM EDTA (Invitrogen, Cat.15575020) and 1% FBS in PBS followed by re-suspending in FACS buffer containing 1:10,000 dilution of DAPI (Biolegend, Cat.422801) nuclear stain. Cells were then processed on a Cytoflex flow cytometer (Beckman Coulter) and analyzed using the FlowJo software package. T cells were gated based on size, shape, viability, and GFP expression. Values that were not determined by the flow cytometer were denoted as “ND”. Table 18 and FIGS. 8A-8B show mean percentage of T cells positive for GFP expression.

TABLE 18
Mean percentage of T cells positive for GFP expression
following genomic editing of AAVS1 with SpyCas9 and AAV

| | Guide | Donor 1 | | | Donor 2 | | |
|---|---|---|---|---|---|---|---|
| | ID | Mean | SD | N | Mean | SD | N |
| Controls | EP Only | 0.03 | ND | 1 | 0.04 | ND | 1 |
| | AAV | 6.81 | 0.18 | 2 | 8.31 | 0.785 | 2 |
| | Only | | | | | | |
| | TRAC | 52.05 | 1.95 | 2 | ND | ND | ND |
| Template A | G000562 | 30.60 | 0.10 | 2 | 41.20 | 13.6 | 2 |
| | G013562 | 65.75 | 0.45 | 2 | 62.35 | 0.95 | 2 |
| | G013563 | 73.15 | 0.25 | 2 | 69.40 | 0.9 | 2 |
| | G013564 | 65.95 | 0.45 | 2 | 61.20 | 0 | 2 |
| | G013582 | 66.30 | 0.10 | 2 | 61.00 | 0.3 | 2 |
| | G013584 | 69.15 | 0.15 | 2 | 62.95 | 3.95 | 2 |
| Template B | AAV | 8.44 | 1.07 | 2 | 9.74 | 0.23 | 2 |
| | Only | | | | | | |
| | G000562 | 34.00 | 0.10 | 2 | 32.75 | 2.35 | 2 |
| | G013559 | 65.55 | 0.35 | 2 | 64.05 | 2.65 | 2 |
| | G013562 | 63.60 | 0.90 | 2 | 65.40 | 0.2 | 2 |
| | G013563 | 69.15 | 0.65 | 2 | 71.35 | 0.35 | 2 |
| | G013564 | 61.50 | 1.10 | 2 | 63.45 | 0.55 | 2 |
| | G013565 | 63.50 | 0.40 | 2 | 65.00 | 1.1 | 2 |
| Template C | AAV | 7.98 | ND | 1 | 10.10 | ND | 1 |
| | Only | | | | | | |
| | G000562 | 57.95 | 0.15 | 2 | 59.10 | 0.9 | 2 |
| | G013515 | 61.15 | 0.65 | 2 | 60.95 | 1.15 | 2 |
| | G013533 | 65.65 | 0.35 | 2 | 65.15 | 0.25 | 2 |
| Template D | AAV | 7.12 | ND | 1 | 10.70 | ND | 1 |
| | Only | | | | | | |
| | G000562 | 29.65 | 1.15 | 2 | 27.50 | 0.7 | 2 |
| | G013519 | 73.35 | 0.45 | 2 | 72.10 | 0.9 | 2 |
| | G013520 | 71.80 | 0.20 | 2 | 72.20 | 1.3 | 2 |
| | G013523 | 70.50 | 0.60 | 2 | 72.05 | 0.55 | 2 |
| Template | AAV | 6.32 | ND | 1 | 6.87 | ND | 1 |
| OG | Only | | | | | | |
| | G000562 | 41.75 | 0.15 | 2 | 37.80 | 0.4 | 2 |
| | G013543 | 65.25 | 0.05 | 2 | 64.40 | 0.2 | 2 |

### Example 7. Orthogonal or Non-Orthogonal Multi-Edits

To assess the editing profile and cell behavior of cells undergoing simultaneous multiple edits using orthogonal editors or other editing schemes, T cells were treated with lipid nanoparticles (LNP) and analyzed for cell expansion, editing, and surface protein expression.

### Example 7.1. T Cell Preparation

Isolated, cryopreserved T cells were thawed on Day 0 in a water bath and plated at a density of 1.5×10\hat{ }6 cells/mL in TCAM media containing CTS OpTmizer T Cell Expansion SFM and T Cell Expansion Supplement (ThermoFisher Cat. A1048501), 1× Penicillin-Streptomycin, 1× Glutamax, 10 mM HEPES, 200 U/mL recombinant human interleukin-2 (Peprotech, Cat. 200-02), 5 ng/ml recombinant human interleukin 7 (Peprotech, Cat. 200-07), and 5 ng/ml recombinant human interleukin 15 (Peprotech, Cat. 200-15) and 2.5% human AB serum (GeminiBio, Cat. 100-512). Biological replicates were performed using isolated T cells from 3 donors.

### Example 7.2. LNP Treatment and Expansion of T Cells

LNPs were generally prepared as described in Example 1. Lipid nanoparticles in this example were prepared with molar ratios of 35 Lipid A: 47.5 cholesterol: 15 DSPC: 2.5 PEG2k-DMG. LNPs were made with a lipid amine to RNA phosphate (N:P) molar ratio of about 6. LNPs were formulated with a single RNA cargo or coformulated with multiple RNA species as described in Table 22. LNPs were delivered to T cells in TCAM media containing ApoE3 (Peprotech, Cat. 350-02) as described in Table 23 and below.

TABLE 22
Lipid Nanoparticles. Cargo mass ratios are listed respectively
to the order in the Cargo column. Dose is measured
as mass of total RNA cargo per unit volume.

| LNP | Dose (ug/ml) | Cargo | Cargo Mass Ratio |
|---|---|---|---|
| 1 | 0.84 | SpBC22n mRNA | n/a |
| 2 | 0.42 | UGI mRNA | n/a |
| 3 | 1.67 | TRAC G023520 + | 1:1.9:14.5:4.8 |
| | | TRBC G023524 + | |
| | | HLA-A G023523 + | |
| | | CIITA G023521 | |
| 4 | 1.25 | Nme2Cas9 mRNA + | 1:2 |
| | | TRAC G021469 | |
| 5 | 0.65 | SpCas9 mRNA + | 1:1 |
| | | CIITA G013675 | |
| 6 | 1.00 | SpCas9 mRNA + | 1:1 |
| | | TRAC G013006 | |
| 7 | 0.65 | SpCas9 mRNA + | 1:1 |
| | | HLA-A G018995 | |
| 8 | 1.00 | SpCas9 mRNA + | 1:1 |
| | | TRBC G016239 | |

Twenty-four hours post thawing (Day 1), cells were harvested and activated with TransAct™ (1:100 dilution, Miltenyi Biotec). LNPs were applied at the doses listed in Table 22 on the schedule provided in Table 23. Between treatments, cells were incubated at 37° C. As indicated in Table 23, on Day 3 Group C, Group D, and Group E were treated with 3×10⁵ GC/cell of AAV to deliver a homology directed repair template encoding a transgenic T cell receptor simultaneous with LNP treatments and with 0.25 uM of Compound 1. T cells were seeded at a density of 1E6 cells/mL for activation (Day 1) and sustained at a density of 0.5×10⁶/cells/mL throughout editing on Days 3-5.

TABLE 23
Order of editing for T cell engineering

| Treatment | Day 1 | Day 3 | Day 4 | Day 5 |
|---|---|---|---|---|
| Group A | none | none | none | none |
| Unedited | | | | |
| Group B | none | LNP1 | none | none |
| Simultaneous | none | LNP2 | none | none |
| Sp Base Editor | none | LNP3 | none | none |
| Group C | none | LNP1 | none | none |
| Simultaneous | none | LNP2 | none | none |
| Sp Base Editor + | none | LNP3 | none | none |
| Nme2Cas9 | none | LNP4 | none | none |
| insertion | none | AVV: TCR | none | none |
| Group D | none | LNP5 | none | none |
| Simultaneous | none | LNP6 | none | none |
| SpCas9 | none | LNP7 | none | none |
| | none | LNP8 | none | none |
| | none | AAV: TCR | none | none |
| Group E | LNP5 | LNP6 | LNP7 | LNP8 |
| Sequential | none | AAV: TCR | none | none |
| SpCas9 | | | | |

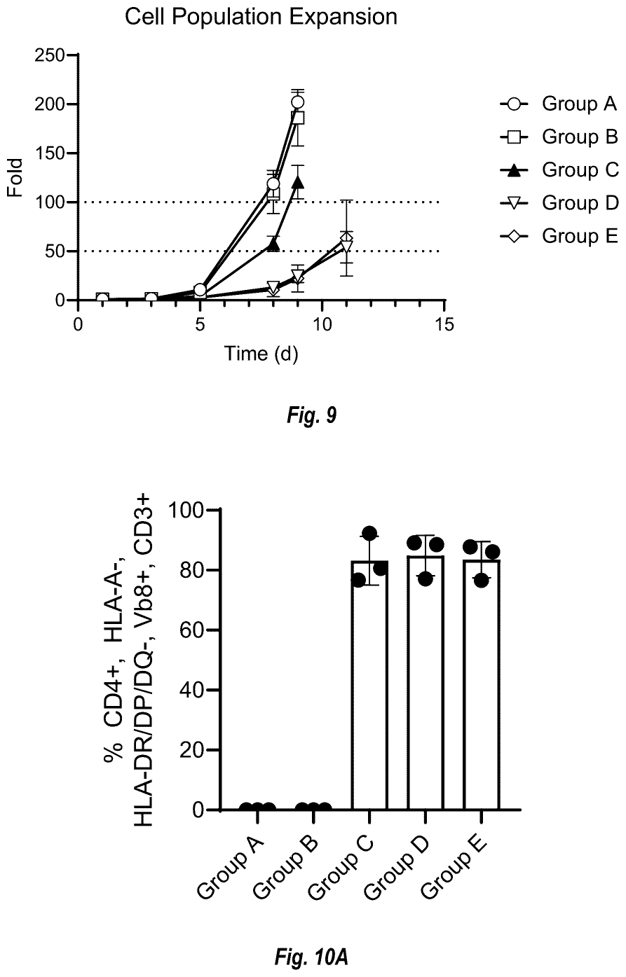

On Day 5 for Groups A, B, C, and D and on Day 6 for Group E cells were washed and transferred to 6 well GREX plates (Wilson Wolf). Media was refreshed on days 7 and 10. Cells were counted using a Cellaca MX (Nexcelom) and fold expansion was calculated by dividing cell yield by the starting material activated on Day 1. Table 24 and FIG. 9 show cell expansion after activation. After 9 days of expansion, Group C with four gene disruptions via base edit and a single DNA cleavage for insertion showed about five-fold better cell expansion compared to the simultaneous multi-cleavage Group D and the sequential multi-cleavage Group E.

TABLE 24
Cell population expansion after indicated growth period in expansion media.

| | Group A | | Group B | | Group C | | Group D | | Group E | |
|---|---|---|---|---|---|---|---|---|---|---|
| Days | Mean | SD | Mean | SD | Mean | SD | Mean | SD | Mean | SD |
| 1 | 1.0 | 0.0 | 1.0 | 0.0 | 1.0 | 0.0 | 1.0 | 0.0 | 1.0 | 0.0 |
| 3 | 1.5 | 0.1 | 1.5 | 0.1 | 1.5 | 0.2 | 1.4 | 0.0 | 1.2 | 0.1 |
| 5 | 10.8 | 1.2 | 8.0 | 0.8 | 4.0 | 0.7 | 2.7 | 0.5 | 2.9 | 0.6 |
| 8 | 118.8 | 13.6 | 108.4 | 20.2 | 57.6 | 7.6 | 13.1 | 4.2 | 10.6 | 7.0 |
| 9 | 202.2 | 9.9 | 186.2 | 28.9 | 120.4 | 17.2 | 24.4 | 6.6 | 22.1 | 13.9 |
| 11 | | | | | | | 54.0 | 16.1 | 63.4 | 38.9 |

On Day 9 or Day 11 of expansion growth, cells were harvested and analyzed by flow cytometry. For flow cytometric analysis, cells were washed in FACS buffer (PBS+2% FBS+2 mM EDTA). Engineered T cells were incubated in a cocktail of antibodies targeting CD4 (Biolegend 317434), CD8 (Biolegend 301046), CD3 (Biolegend 300430), Vb8 (Biolegend 348104), HLA-A2 (Biolegend 343320) or HLA-A3 (Fisher 50-112-3136) and HLA-DR, DP, DQ (Biolegend 361712). T cells were subsequently washed and analyzed on a Cytoflex instrument (Beckman Coulter). Data analysis was performed using FlowJo software package (v.10.6.1). T cells were gated on size, single cells, CD4 or CD8 expression, and expression of markers indicated in Table 25. Flow cytometry data shown in Table 25 and FIGS. 10A-B. Vb8+ levels indicate insertion and expression of the transgenic TCR in Groups C, D, and E. Efficient CIITA disruption in Groups B, C, D, and E is indicated by the increase in HLA-DR, DP, DQ-cells compared to Group A, as CIITA is a transcription factor that controls the expression of these surface proteins. Efficient HLA-A disruption is indicated by the increased HLA-A-population in in Groups B, C, D, and E compared to Group A. Efficient disruption of the TRAC and TRBC loci is indicated by the decrease in CD3+Vb8− in Groups B, C, D, and E compared to Group A. The percent of fully edited product was gated as HLA-A-, HLA-DR/DP/DQ-, CD3+, Vb8+.

TABLE 25
Mean percent of T cells displaying cell surface phenotype

| T cell | Additional | Group A | | Group B | | Group C | | Group D | | Group E | |
|---|---|---|---|---|---|---|---|---|---|---|---|
| type | Markers | Mean | SD | Mean | SD | Mean | SD | Mean | SD | Mean | SD |
| CD4+ | HLA-A− | 0.3 | 0.3 | 99.5 | 0.3 | 99.9 | 0.2 | 95.0 | 5.1 | 95.4 | 4.7 |
| | HLA- | 50.5 | 15.2 | 97.6 | 1.0 | 98.7 | 0.8 | 97.8 | 0.4 | 98.9 | 0.4 |
| | DR/DP/DQ− | | | | | | | | | | |
| | CD3 + Vb8− | 93.7 | 0.9 | 0.4 | 0.5 | 0.1 | 0.1 | 0.2 | 0.1 | 0.2 | 0.3 |
| | Vb8+ | 6.0 | 1.0 | 0.0 | 0.0 | 83.9 | 7.9 | 90.6 | 1.7 | 88.0 | 1.9 |
| | HLA-A−, | 0.0 | 0.0 | 0.0 | 0.0 | 83.2 | 8.1 | 84.9 | 6.8 | 83.5 | 6.0 |
| | HLA- | | | | | | | | | | |
| | DR/DP/DQ−, | | | | | | | | | | |
| | Vb8+, CD3+ | | | | | | | | | | |
| CD8+ | HLA-A− | 0.3 | 0.5 | 99.3 | 0.3 | 99.7 | 0.5 | 93.6 | 9.1 | 95.7 | 6.1 |
| | HLA- | 23.8 | 15.3 | 94.7 | 3.3 | 96.6 | 3.0 | 94.5 | 2.2 | 97.8 | 0.8 |
| | DR/DP/DQ− | | | | | | | | | | |
| | CD3+ Vb8− | 94.7 | 1.5 | 0.8 | 0.6 | 0.1 | 0.1 | 0.2 | 0.3 | 0.0 | 0.1 |
| | Vb8+ | 5.1 | 1.4 | 0.1 | 0.1 | 82.7 | 8.4 | 90.2 | 1.8 | 87.1 | 4.3 |
| | HLA-A−, | 0.0 | 0.0 | 0.0 | 0.1 | 80.2 | 9.4 | 81.2 | 7.1 | 82.2 | 8.6 |
| | HLA- | | | | | | | | | | |
| | DR/DP/DQ−, | | | | | | | | | | |
| | Vb8+, CD3+ | | | | | | | | | | |

### Example 8. Functional Characterization of Orthogonally Engineered T Cells

To assess the functionality of cells engineered with orthogonal editors, cells were analyzed for editing, surface protein expression, and cytotoxicity.

### Example 8.1. T Cell Preparation

Isolated, cryopreserved T cells were thawed in a water bath and plated at a density of 1.5×10⁶ cells/mL in TCAM media containing CTS OpTmizer T Cell Expansion SFM and T Cell Expansion Supplement (ThermoFisher Cat. A1048501), 1× Penicillin-Streptomycin, 1× Glutamax, 10 mM HEPES, 200 U/mL recombinant human interleukin-2 (Peprotech, Cat. 200-02), 5 ng/ml recombinant human interleukin 7 (Peprotech, Cat. 200-07), and 5 ng/ml recombinant human interleukin 15 (Peprotech, Cat. 200-15) and 2.5% human AB serum (GeminiBio, Cat. 100-512).

### Example 8.2. T Cell Engineering

LNPs were generally prepared as described in Example 1. Lipid nanoparticles in this example were prepared with molar ratios of 35 Lipid A: 47.5 cholesterol: 15 DSPC: 2.5 PEG2k-DMG. LNPs were made with a lipid amine to RNA phosphate (N:P) molar ratio of about 6. LNPs were formulated with a single RNA species or coformulated with multiple RNA species as described in Table 26. LNPs were delivered to T cells in TCAM media containing ApoE3 (Peprotech, Cat. 350-02) as described in Table 27 and below.

TABLE 26
Lipid Nanoparticles. Cargo mass ratios are listed respectively
to the order in the Cargo column. Dose is measured
as mass of total RNA cargo per unit volume.

| LNP | Dose (ug/ml) | Cargo | Cargo MassRatio |
|---|---|---|---|
| 1 | 0.25 | Spy BC22 mRNA | n/a |
| 2 | 0.5 | UGI mRNA | n/a |
| 3 | 1.67 | TRAC G027891 | 4.5/8.7/21.4/65.4 |
| | | TRBC G027904 | |
| | | CIITA G028535 | |
| | | HLA-A G028536 | |
| 4 | 1.25 | Nme2Cas9 mRNA + TRAC | 1:2 |
| | | G021469 | |

On Day 1 (e.g. 24 hours post thaw) cells were harvested and activated with TransAct™ (1:100 dilution, Miltenyi Biotec). LNPs were applied at the doses listed in Table 26 on Day 3 to the treatment groups as listed in Table 27. Group C samples were treated with 3E+5 GC/mL of AAV encoding a transgenic T cell receptor (AAV 1760 with HD1 Insert) and with 0.5 uM of Compound 1 simultaneous with LNP treatments.

TABLE 27
Editing regime for T cell engineering.

| Treatment | Day 3 |
|---|---|
| Group A | none |
| Unedited | |
| Group B | LNP1 |
| Sp Base Editor | LNP2 |
| | LNP3 |
| Group C | LNP1 |
| Sp Base Editor + | LNP2 |
| Nme2 Cas9 | LNP3 |
| insertion | LNP4 |
| | AAV |

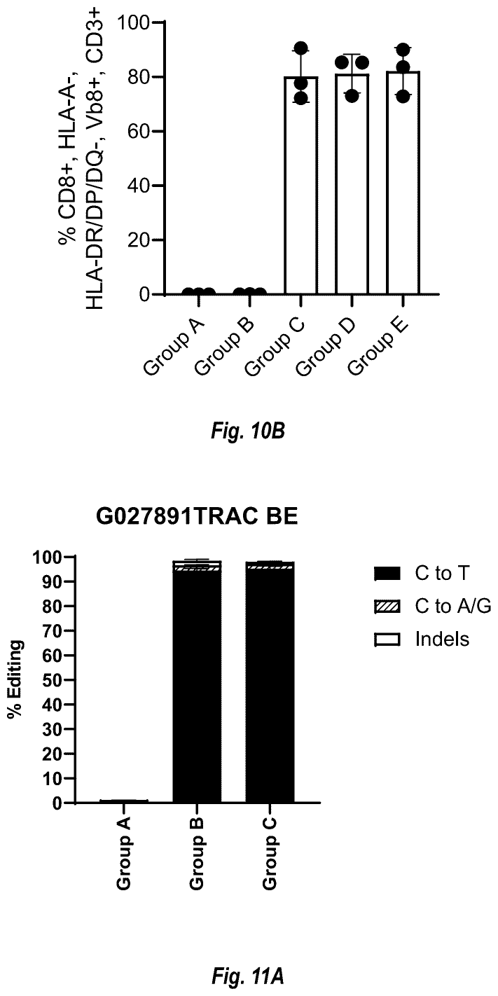

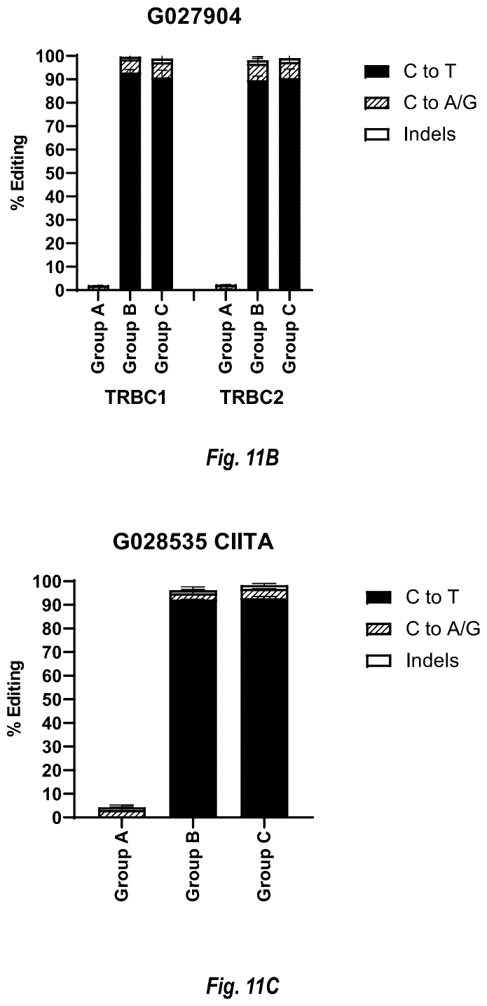

On Day 4, cells were transferred to 6 well GREX plates (Wilson Wolf) with T cell expansion media (TCEM): CTS OpTmizer (Thermofisher, Cat. A3705001) supplemented with 5% human AB serum, 1× GlutaMAX (Thermofisher, Cat. 35050061), 10 mM HEPES, 200 U/mL IL-2 (Peprotech, Cat. 200-02), IL-7 (Peprotech, Cat. 200-07), and IL-15 (Peprotech, Cat. 200-15). Media was refreshed regularly. On Day 7, a portion of cells were harvested for sequencing analysis at TRBC1, TRBC2 and CIITA loci. NGS analysis was performed with technical triplicates as described in Example 1. Table 28 and FIG. 11A-11C show mean percent editing for these cells.

TABLE 28
Mean percent editing. “Cas9” indicates guides specific
to Nme2Cas9. “BE” indicates a guide designed for SpyBC22n
base editor. “n/a” indicates standard deviation is not applicable.

| | | C to T | | | C to A/G | | | Indels | | |
|---|---|---|---|---|---|---|---|---|---|---|
| Edit | Treatment | Mean | SD | n | Mean | SD | n | Mean | SD | n |
| G027891 | Group A | 0.3 | 0.1 | 5 | 0.7 | 0.1 | 5 | 0.2 | 0.0 | 5 |
| TRAC BE | Group B | 94.5 | 0.8 | 5 | 2.2 | 0.2 | 5 | 1.8 | 0.5 | 5 |
| | Group C | 95.3 | 1.0 | 6 | 1.9 | 0.4 | 6 | 0.9 | 0.2 | 6 |
| G027904 | Group A | 0.3 | 0.1 | 3 | 1.6 | 0.1 | 3 | 0.2 | 0.0 | 3 |
| TRBC1 | Group B | 92.9 | 1.2 | 3 | 5.6 | 0.5 | 3 | 1.2 | 0.7 | 3 |
| BE | Group C | 90.8 | 3.1 | 4 | 6.5 | 3.1 | 4 | 1.6 | 1.9 | 4 |
| G027904 | Group A | 0.3 | 0.3 | 4 | 1.9 | 0.2 | 3 | 0.2 | 0.0 | 3 |
| TRBC2 | Group B | 89.6 | 1.8 | 4 | 7.0 | 3.0 | 4 | 1.5 | 0.8 | 4 |
| BE | Group C | 90.5 | 3.9 | 5 | 6.9 | 2.8 | 5 | 1.6 | 1.7 | 5 |
| G028535 | Group A | 0.3 | n/a | 1 | 2.8 | 1.4 | 3 | 1.2 | 1.0 | 3 |
| CIITA | Group B | 92.3 | 0.1 | 2 | 2.6 | 1.6 | 4 | 1.3 | 1.4 | 4 |
| BE | Group C | 92.9 | 0.6 | 3 | 3.9 | 0.1 | 3 | 1.6 | 0.6 | 3 |

On Day 11, cells were counted using a Cellaca MX (Nexcelom) in technical triplicates and fold expansion was calculated by dividing cell yield by the amount of edited cells on Day 3. Table 29 shows fold cell expansion.

TABLE 29
Fold expansion of cell population.

| Treatment | Fold | SD | n |
|---|---|---|---|
| Group A | 218 | 12 | 6 |
| Group B | 173 | 9 | 6 |
| Group C | 110 | 4 | 6 |

### Example 8.3 Flow Cytometry

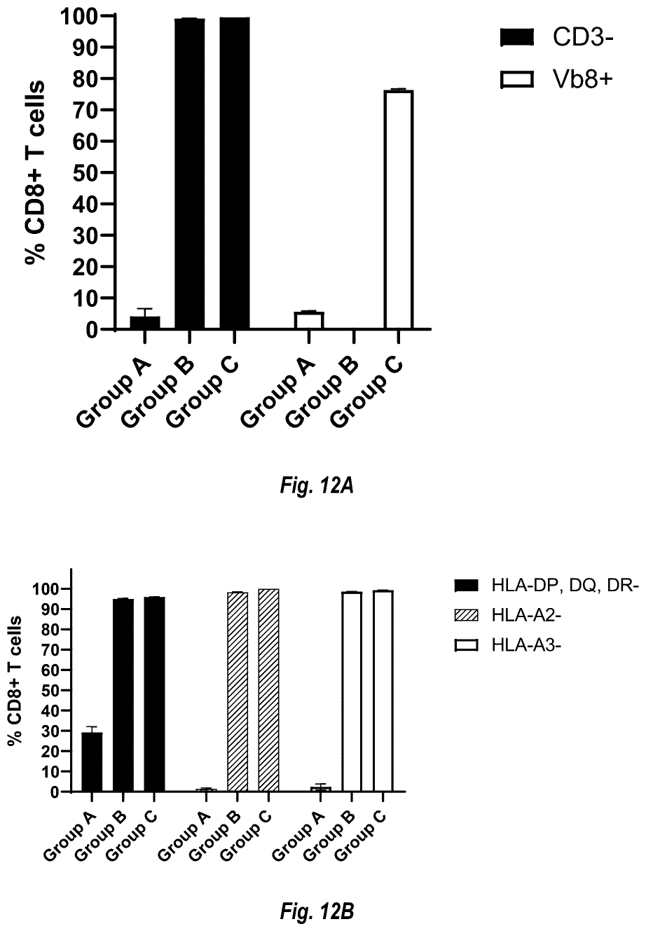

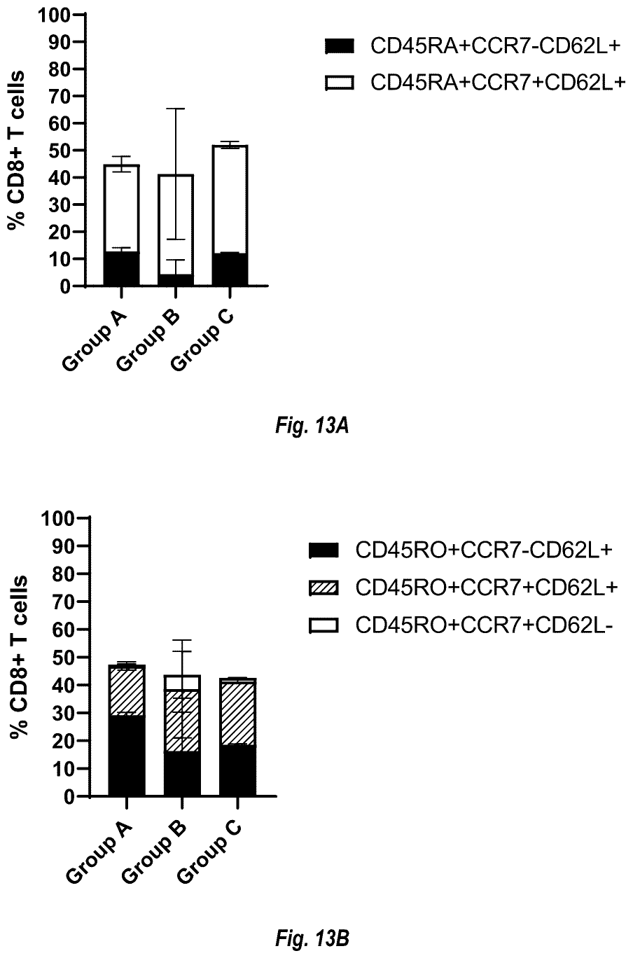

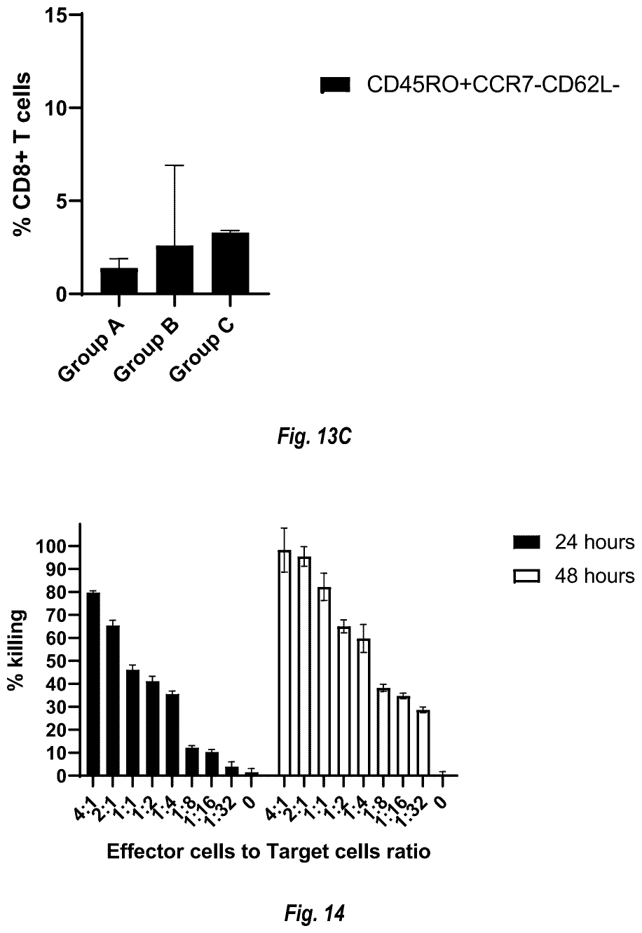

On Day 11, cells were harvested for analysis by flow cytometry (technical triplicates). For flow cytometric analysis, cells were washed in FACS buffer (PBS+2% FBS+2 mM EDTA). Engineered T cells were incubated in a cocktail of antibodies targeting CD4 (Biolegend 317434), CD8 (Biolegend 301046), CD3 (Biolegend 300430), Vb8 (Biolegend 348104), HLA-A2 (Biolegend 343320), HLA-A3 (Thermo Fisher Scientific, 501122136), HLA-DR, DP, DQ (Biolegend 361712), CD45RA (Biolegend, 304134), CD45RO (Biolegend, 304230), CD62L (Biolegend, 304820), CCR7 (Biolegend, 353214) and ViaKrome 808 Fixable Viablility Dye (Beckman Coulter, C36628). T cells were subsequently washed and analyzed on a Cytoflex instrument (Beckman Coulter). Data analysis was performed using FlowJo software package (v.10.6.1). T cells were gated on size, viability, CD4 or CD8 expression, and expression of markers indicated in Table 30. Flow cytometry data for CD8+ cells are shown in Table 30 and FIGS. 12A-12B and 13A-13C. Results for CD4+ cells were similar to CD8+ cells. HLA-A2-, HLA-A3- and HLA-DP, DQ, DQ-cell populations indicate efficient disruption of HLA-A locus, and CIITA locus, respectively in Group B and Group C compared to unedited control Group A. To determine endogenous TCR disruption resulting from TRAC and TRBC loci editing (referred to as “CD3−” in Table 30 and FIG. 12A) the % of cells expressing endogenous TCR (CD3+Vb8−) was subtracted from 1000. The increase in the Vb8+ cell population in Group C compared to Group A and Group B indicates effective insertion of the transgenic TCR. The similarity in CD45RA+ and CD45RO+ phenotypes between Group A and Group C shown in Table 30 and FIGS. 13A-13C indicates that the central memory cell, central memory stem cell, and effector memory phenotypes are intact for the engineered cells.

TABLE 30
Mean percent of CD8+ T cells displaying cell surface phenotype.

| Phenotype | Treatment | Mean | SD | n |
|---|---|---|---|---|
| “CD3−” | Group A | 4.1 | 2.5 | 6 |
| (100%-CD3+Vb8−) | Group B | 99.1 | 0.1 | 6 |
| | Group C | 99.5 | 0.0 | 6 |
| HLA-DP, DQ, DR− | Group A | 29.2 | 2.8 | 6 |
| | Group B | 94.9 | 0.3 | 6 |
| | Group C | 95.9 | 0.1 | 6 |
| HLA-A2− | Group A | 1.5 | 0.4 | 6 |
| | Group B | 98.2 | 0.2 | 6 |
| | Group C | 100.0 | 0.0 | 6 |
| HLA-A3− | Group A | 2.4 | 1.5 | 6 |
| | Group B | 98.5 | 0.1 | 6 |
| | Group C | 99.2 | 0.1 | 6 |
| Vb8+ | Group A | 5.6 | 0.3 | 6 |
| | Group B | 0.0 | 0.0 | 6 |
| | Group C | 76.4 | 0.3 | 6 |
| CD45RA+CCR7−CD62L+ | Group A | 12.8 | 1.3 | 6 |
| | Group B | 4.4 | 5.2 | 6 |
| | Group C | 12.1 | 0.3 | 6 |
| CD45RA+CCR7+CD62L+ | Group A | 32.1 | 2.9 | 6 |
| | Group B | 36.9 | 24.1 | 6 |
| | Group C | 39.9 | 1.3 | 6 |
| CD45RO+CCR7−CD62L+ | Group A | 29.2 | 1.0 | 6 |
| | Group B | 16.3 | 14.0 | 6 |
| | Group C | 18.5 | 0.4 | 6 |
| CD45RO+CCR7+CD62L+ | Group A | 17.7 | 1.5 | 6 |
| | Group B | 22.3 | 17.6 | 6 |
| | Group C | 22.8 | 0.7 | 6 |
| CD45RO+CCR7+CD62L− | Group A | 0.5 | 0.2 | 6 |
| | Group B | 5.1 | 8.4 | 6 |
| | Group C | 1.3 | 0.1 | 6 |
| CD45RO+CCR7−CD62L− | Group A | 1.4 | 0.5 | 6 |
| | Group B | 2.6 | 4.3 | 6 |
| | Group C | 3.3 | 0.1 | 6 |

### Example 8.4. Luciferase-Based Cytotoxicity Analysis

Treatment Group C cells were thawed and cultured overnight. Cells were co-cultured at an effector-to-target ratios indicated in Table 31 with 697 Luc GFP+ cells. Co-cultures were performed in a cytokine-free media composed of CTS OpTmizer T Cell Expansion SFM and T Cell Expansion Supplement (ThermoFisher Cat. A1048501), 2.5% human AB serum (GeminiBio, Cat. 100-512) 1× Penicillin-Streptomycin, 1× Glutamax, and 10 mM HEPES.

After 24 and 48 hours, the amount of luciferase enzyme produced by live 697 cells, which is inversely proportional to engineered T cytotoxicity, was measured by the Bright-Glo assay (Promega Cat. E2620) following the manufacturer's instructions. Luminescence was measured using a CLARIOstar Plus (BMG LabTech Sr. No. 430-4346) plate reader. The percentage specific killing was calculated as 100%−(100*experimental well luminescence/average target only well luminescence). Table 31 and FIG. 14 show mean percent cell killing at various effector to target ratios.

TABLE 31
Mean percent target cell killing by engineered T cells.

| | 24 hours | | | 48 hours | | |
|---|---|---|---|---|---|---|
| E:T | Mean | SD | n | Mean | SD | n |
| 4:1 | 79.7 | 0.7 | 3 | 98.2 | 9.6 | 3 |
| 2:1 | 65.4 | 2.2 | 3 | 95.4 | 4.3 | 3 |
| 1:1 | 46.1 | 2.1 | 3 | 82.2 | 5.9 | 3 |
| 1:2 | 41.1 | 2.2 | 3 | 65.0 | 2.8 | 3 |
| 1:4 | 35.6 | 1.2 | 3 | 59.7 | 6.1 | 3 |
| 1:8 | 12.2 | 0.9 | 3 | 38.2 | 1.6 | 3 |
| 1:16 | 10.3 | 1.1 | 3 | 34.7 | 1.2 | 3 |
| 1:32 | 3.9 | 2.1 | 3 | 28.7 | 1.3 | 3 |
| Target only | 1.5 | 1.7 | 3 | −0.3 | 2.0 | 3 |

### Example 9. Editing with Select Guides Using SpCas9 and Nme2 Base Editor

To assess editing with select guides using SpyCas9 and Nme2 base editor, engineered cells were evaluated by flow cytometry and NGS. This example includes selected guide concentrations and Nme2 BC22 mRNA concentrations.

### Example 9.1. T Cell Preparation

Isolated, cryopreserved T cells were thawed in a water bath and plated at a density of 1×10⁶ cells/mL in TCAM media containing CTS OpTmizer T Cell Expansion SFM and T Cell Expansion Supplement (ThermoFisher Cat. A1048501), 1× Penicillin-Streptomycin, 1× Glutamax, 10 mM HEPES, 200 U/mL recombinant human interleukin-2 (Peprotech, Cat. 200-02), 5 ng/mL recombinant human interleukin 7 (Peprotech, Cat. 200-07), and 5 ng/mL recombinant human interleukin 15 (Peprotech, Cat. 200-15) and 2.5% human AB serum (GeminiBio, Cat. 100-512). Technical replicates were prepared using isolated T cells from multiple donors. Data shown is from a selected donor.

### Example 9.2. T Cell Engineering

LNPs were generally prepared as described in Example 1. Lipid nanoparticles in this example were prepared with molar ratios of 35 Lipid A: 47.5 cholesterol: 15 DSPC: 2.5 PEG2k-DMG. LNPs were made with a lipid amine to RNA phosphate (N:P) molar ratio of about 6. LNPs were formulated with a single RNA species or coformulated with multiple RNA species as described in Table 32. LNPs were delivered to T cells in TCAM media containing ApoE3 (Peprotech, Cat. 350-02) as described in Table 32.

TABLE 32
Lipid Nanoparticles. Cargo mass ratios are listed respectively
to the order in the Cargo column. Dose is measured
as mass of total RNA cargo per unit volume.

| LNP | Dose (ug/ml) | Cargo | Cargo Mass Ratio |
|---|---|---|---|
| 1 | — | NmeBC22n mRNA | n/a |
| 2 | 0.5 | UGI mRNA | n/a |
| 3 | 0.32 | G028986(TRBC) | n/a |
| 4 | 0.68 | G026584 (CIITA) | n/a |
| 5 | 0.21 | G028907 (HLA-A/ | n/a |
| | | HLA-B) | |
| 6 | 1 | Spy Cas9 mRNA + | 1:1 |
| | | G013006 (TRAC) | |

On Day 1 (e.g., about 24 hours post thaw) cells were harvested and activated with TransAct™ (1:100 dilution, Miltenyi Biotec). LNPs were applied at the doses listed in Table 32 on Day 3 along with 0.5 uM of Compound 1.

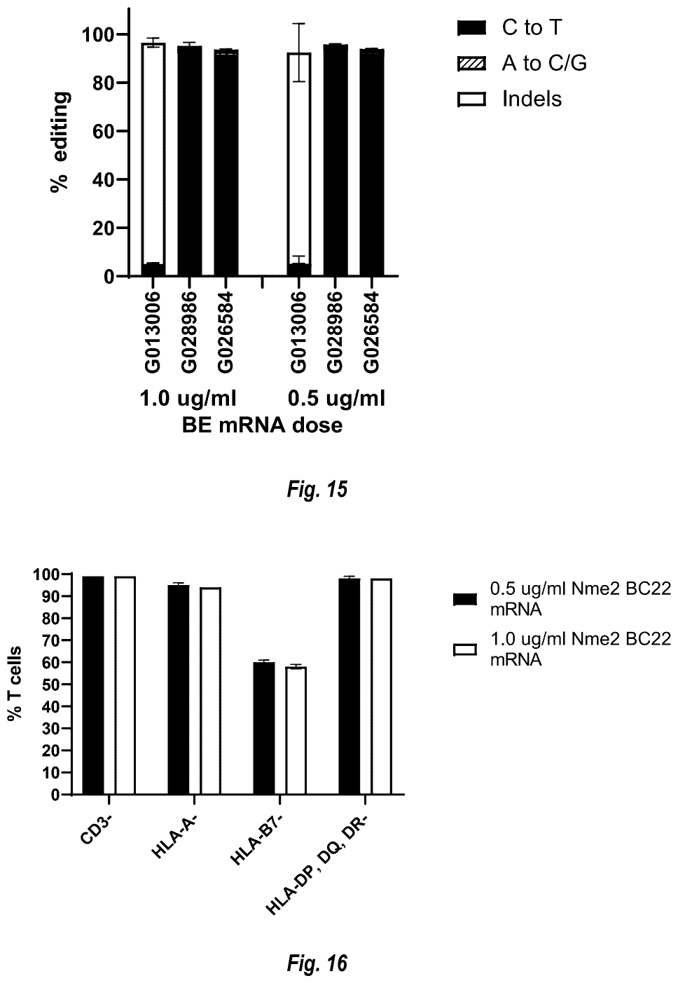

Beginning on Day 4, cells were split and media refreshed regularly. On Day 7, a portion of cells were harvested for sequencing analysis at TRAC, TRBC1, TRBC2 and CIITA loci. NGS analysis was performed as described in Example 1. Table 33 and FIG. 15 show mean percent editing for these cells.

TABLE 33
Mean percent editing.

| | | C to T | | | C to A/G | | | Indels | | |
|---|---|---|---|---|---|---|---|---|---|---|
| Guide | BE mRNA | Mean | SD | n | Mean | SD | n | Mean | SD | n |
| G013006 | 1.0 ug/mL | 4.9 | 0.6 | 3 | 0.2 | 0.1 | 3 | 91.4 | 1.9 | 3 |
| TRAC Cas9 | 0.5 ug/mL | 5.0 | 3.4 | 3 | 0.2 | 0.1 | 3 | 87.2 | 12.0 | 3 |
| G028986 | 1.0 ug/mL | 93.3 | 1.5 | 3 | 0.9 | 0.5 | 3 | 1.0 | 1.4 | 3 |
| TRBC1 BE | 0.5 ug/mL | 94.4 | 0.2 | 3 | 1.0 | 0.6 | 3 | 0.3 | 0.3 | 3 |
| G026584 | 1.0 ug/mL | 91.6 | 0.8 | 3 | 1.4 | 0.2 | 3 | 0.6 | 0.3 | 3 |
| CIITA BE | 0.5 ug/mL | 91.8 | 1.3 | 3 | 1.2 | 0.3 | 3 | 0.9 | 0.2 | 3 |

### Example 9.3 Flow Cytometry

On Day 9, cells were harvested for analysis by flow cytometry. For flow cytometric analysis, cells were washed in FACS buffer (PBS+2% FBS+2 mM EDTA). Engineered T cells were incubated in a cocktail of antibodies targeting CD4 (Biolegend 317434), CD8 (Biolegend 301046), CD3 (Biolegend 300430), HLA-A2 (Biolegend 343320), HLA-B7 (Miltenyi Biotec, 130-120-234), HLA-DR, DP, DQ (Biolegend 361712), and ViaKrome 808 Fixable Viablility Dye (Beckman Coulter, C36628). T cells were subsequently washed and analyzed on a Cytoflex instrument (Beckman Coulter). Data analysis was performed using FlowJo software package (v.10.6.1). T cells were gated on live single cells, CD8+ expression, and expression of markers indicated in Table 34. Flow cytometry data for CD8+ cells are shown in Table 34 and FIG. 16. CD3-, HLA-A2-, HLA-B7-, and HLA-DP, DQ, DQ-cell populations indicate efficient disruption of TRAC, TRBC1, and TRBC2 loci, HLA-A locus, HLA-B locus, and CIITA locus, respectively.

TABLE 34
Mean percent of CD8+ T cells
negative for surface protein expression.

| | 0.5 ug/ml Nme2 | | | 1.0 ug/ml Nme2 | | |
|---|---|---|---|---|---|---|
| | BC22 mRNA | | | BC22 mRNA | | |
| Phenotype | Mean | SD | n | Mean | SD | n |
| CD3− | 99 | 0 | 3 | 99 | 0 | 3 |
| HLA-A− | 95 | 1 | 3 | 94 | 0 | 3 |
| HLA-B7− | 60 | 1 | 3 | 58 | 1 | 3 |
| HLA-DP, DQ, DR− | 98 | 1 | 3 | 98 | 0 | 3 |

### Table of Sequences

In the following table, the terms “mA,” “mC,” “mU,” or “mG” are used to denote a nucleotide that has been modified with 2′-O-Me.

In the following table, each “N” is used to independently denote any nucleotide (e.g., A, U, T, C, G). In certain embodiments, the nucleotide is an unmodified RNA nucleotide residue, i.e., a ribose sugar and a phosphodiester backbone.

In the following table, a “*” is used to denote a PS modification. In this application, the terms A*, C*, U*, or G* may be used to denote a nucleotide that is linked to the next (e.g., 3′) nucleotide with a PS bond.

It is understood that if a DNA sequence (comprising Ts) is referenced with respect to an RNA, then Ts should be replaced with Us (which may be modified or unmodified depending on the context), and vice versa.

In the following table, single amino acid letter code is used to provide peptide sequences.

| SEQ | | |
|---|---|---|
| ID | | |
| NO | Description | Sequence |
| 1 | mRNA encoding | GGGAAGCUCAGAAUAAACGCUCAACUUUGGCCGGAUCUGCCACCA |
| | SpyCas9 BC22n | UGGAGGCCUCCCCCGCCUCCGGCCCCCGGCACCUGAUGGACCCCC |
| | | ACAUCUUCACCUCCAACUUCAACAACGGCAUCGGCCGGCACAAGA |
| | | CCUACCUGUGCUACGAGGUGGAGCGGCUGGACAACGGCACCUCCG |
| | | UGAAGAUGGACCAGCACCGGGGCUUCCUGCACAACCAGGCCAAGA |
| | | ACCUGCUGUGCGGCUUCUACGGCCGGCACGCCGAGCUGCGGUUCC |
| | | UGGACCUGGUGCCCUCCCUGCAGCUGGACCCCGCCCAGAUCUACC |
| | | GGGUGACCUGGUUCAUCUCCUGGUCCCCCUGCUUCUCCUGGGGCU |
| | | GCGCCGGCGAGGUGCGGGCCUUCCUGCAGGAGAACACCCACGUGC |
| | | GGCUGCGGAUCUUCGCCGCCCGGAUCUACGACUACGACCCCCUGU |
| | | ACAAGGAGGCCCUGCAGAUGCUGCGGGACGCCGGCGCCCAGGUGU |
| | | CCAUCAUGACCUACGACGAGUUCAAGCACUGCUGGGACACCUUCG |
| | | UGGACCACCAGGGCUGCCCCUUCCAGCCCUGGGACGGCCUGGACG |
| | | AGCACUCCCAGGCCCUGUCCGGCCGGCUGCGGGCCAUCCUGCAGA |
| | | ACCAGGGCAACUCCGGCUCCGAGACCCCCGGCACCUCCGAGUCCG |
| | | CCACCCCCGAGUCCGACAAGAAGUACUCCAUCGGCCUGGCCAUCG |
| | | GCACCAACUCCGUGGGCUGGGCCGUGAUCACCGACGAGUACAAGG |
| | | UGCCCUCCAAGAAGUUCAAGGUGCUGGGCAACACCGACCGGCACU |
| | | CCAUCAAGAAGAACCUGAUCGGCGCCCUGCUGUUCGACUCCGGCG |
| | | AGACCGCCGAGGCCACCCGGCUGAAGCGGACCGCCCGGCGGCGGU |
| | | ACACCCGGCGGAAGAACCGGAUCUGCUACCUGCAGGAGAUCUUCU |
| | | CCAACGAGAUGGCCAAGGUGGACGACUCCUUCUUCCACCGGCUGG |
| | | AGGAGUCCUUCCUGGUGGAGGAGGACAAGAAGCACGAGCGGCAC |
| | | CCCAUCUUCGGCAACAUCGUGGACGAGGUGGCCUACCACGAGAAG |
| | | UACCCCACCAUCUACCACCUGCGGAAGAAGCUGGUGGACUCCACC |
| | | GACAAGGCCGACCUGCGGCUGAUCUACCUGGCCCUGGCCCACAUG |
| | | AUCAAGUUCCGGGGCCACUUCCUGAUCGAGGGCGACCUGAACCCC |
| | | GACAACUCCGACGUGGACAAGCUGUUCAUCCAGCUGGUGCAGACC |
| | | UACAACCAGCUGUUCGAGGAGAACCCCAUCAACGCCUCCGGCGUG |
| | | GACGCCAAGGCCAUCCUGUCCGCCCGGCUGUCCAAGUCCCGGCGG |
| | | CUGGAGAACCUGAUCGCCCAGCUGCCCGGCGAGAAGAAGAACGGC |
| | | CUGUUCGGCAACCUGAUCGCCCUGUCCCUGGGCCUGACCCCCAAC |
| | | UUCAAGUCCAACUUCGACCUGGCCGAGGACGCCAAGCUGCAGCUG |
| | | UCCAAGGACACCUACGACGACGACCUGGACAACCUGCUGGCCCAG |
| | | AUCGGCGACCAGUACGCCGACCUGUUCCUGGCCGCCAAGAACCUG |
| | | UCCGACGCCAUCCUGCUGUCCGACAUCCUGCGGGUGAACACCGAG |
| | | AUCACCAAGGCCCCCCUGUCCGCCUCCAUGAUCAAGCGGUACGAC |
| | | GAGCACCACCAGGACCUGACCCUGCUGAAGGCCCUGGUGCGGCAG |
| | | CAGCUGCCCGAGAAGUACAAGGAGAUCUUCUUCGACCAGUCCAAG |
| | | AACGGCUACGCCGGCUACAUCGACGGCGGCGCCUCCCAGGAGGAG |
| | | UUCUACAAGUUCAUCAAGCCCAUCCUGGAGAAGAUGGACGGCACC |
| | | GAGGAGCUGCUGGUGAAGCUGAACCGGGAGGACCUGCUGCGGAA |
| | | GCAGCGGACCUUCGACAACGGCUCCAUCCCCCACCAGAUCCACCU |
| | | GGGCGAGCUGCACGCCAUCCUGCGGCGGCAGGAGGACUUCUACCC |
| | | CUUCCUGAAGGACAACCGGGAGAAGAUCGAGAAGAUCCUGACCU |
| | | UCCGGAUCCCCUACUACGUGGGCCCCCUGGCCCGGGGCAACUCCC |
| | | GGUUCGCCUGGAUGACCCGGAAGUCCGAGGAGACCAUCACCCCCU |
| | | GGAACUUCGAGGAGGUGGUGGACAAGGGCGCCUCCGCCCAGUCCU |
| | | UCAUCGAGCGGAUGACCAACUUCGACAAGAACCUGCCCAACGAGA |
| | | AGGUGCUGCCCAAGCACUCCCUGCUGUACGAGUACUUCACCGUGU |
| | | ACAACGAGCUGACCAAGGUGAAGUACGUGACCGAGGGCAUGCGG |
| | | AAGCCCGCCUUCCUGUCCGGCGAGCAGAAGAAGGCCAUCGUGGAC |
| | | CUGCUGUUCAAGACCAACCGGAAGGUGACCGUGAAGCAGCUGAA |
| | | GGAGGACUACUUCAAGAAGAUCGAGUGCUUCGACUCCGUGGAGA |
| | | UCUCCGGCGUGGAGGACCGGUUCAACGCCUCCCUGGGCACCUACC |
| | | ACGACCUGCUGAAGAUCAUCAAGGACAAGGACUUCCUGGACAAC |
| | | GAGGAGAACGAGGACAUCCUGGAGGACAUCGUGCUGACCCUGAC |
| | | CCUGUUCGAGGACCGGGAGAUGAUCGAGGAGCGGCUGAAGACCU |
| | | ACGCCCACCUGUUCGACGACAAGGUGAUGAAGCAGCUGAAGCGGC |
| | | GGCGGUACACCGGCUGGGGCCGGCUGUCCCGGAAGCUGAUCAACG |
| | | GCAUCCGGGACAAGCAGUCCGGCAAGACCAUCCUGGACUUCCUGA |
| | | AGUCCGACGGCUUCGCCAACCGGAACUUCAUGCAGCUGAUCCACG |
| | | ACGACUCCCUGACCUUCAAGGAGGACAUCCAGAAGGCCCAGGUGU |
| | | CCGGCCAGGGCGACUCCCUGCACGAGCACAUCGCCAACCUGGCCG |
| | | GCUCCCCCGCCAUCAAGAAGGGCAUCCUGCAGACCGUGAAGGUGG |
| | | UGGACGAGCUGGUGAAGGUGAUGGGCCGGCACAAGCCCGAGAAC |
| | | AUCGUGAUCGAGAUGGCCCGGGAGAACCAGACCACCCAGAAGGGC |
| | | CAGAAGAACUCCCGGGAGCGGAUGAAGCGGAUCGAGGAGGGCAU |
| | | CAAGGAGCUGGGCUCCCAGAUCCUGAAGGAGCACCCCGUGGAGAA |
| | | CACCCAGCUGCAGAACGAGAAGCUGUACCUGUACUACCUGCAGAA |
| | | CGGCCGGGACAUGUACGUGGACCAGGAGCUGGACAUCAACCGGCU |
| | | GUCCGACUACGACGUGGACCACAUCGUGCCCCAGUCCUUCCUGAA |
| | | GGACGACUCCAUCGACAACAAGGUGCUGACCCGGUCCGACAAGAA |
| | | CCGGGGCAAGUCCGACAACGUGCCCUCCGAGGAGGUGGUGAAGA |
| | | AGAUGAAGAACUACUGGCGGCAGCUGCUGAACGCCAAGCUGAUC |
| | | ACCCAGCGGAAGUUCGACAACCUGACCAAGGCCGAGCGGGGCGGC |
| | | CUGUCCGAGCUGGACAAGGCCGGCUUCAUCAAGCGGCAGCUGGUG |
| | | GAGACCCGGCAGAUCACCAAGCACGUGGCCCAGAUCCUGGACUCC |
| | | CGGAUGAACACCAAGUACGACGAGAACGACAAGCUGAUCCGGGA |
| | | GGUGAAGGUGAUCACCCUGAAGUCCAAGCUGGUGUCCGACUUCC |
| | | GGAAGGACUUCCAGUUCUACAAGGUGCGGGAGAUCAACAACUAC |
| | | CACCACGCCCACGACGCCUACCUGAACGCCGUGGUGGGCACCGCC |
| | | CUGAUCAAGAAGUACCCCAAGCUGGAGUCCGAGUUCGUGUACGG |
| | | CGACUACAAGGUGUACGACGUGCGGAAGAUGAUCGCCAAGUCCG |
| | | AGCAGGAGAUCGGCAAGGCCACCGCCAAGUACUUCUUCUACUCCA |
| | | ACAUCAUGAACUUCUUCAAGACCGAGAUCACCCUGGCCAACGGCG |
| | | AGAUCCGGAAGCGGCCCCUGAUCGAGACCAACGGCGAGACCGGCG |
| | | AGAUCGUGUGGGACAAGGGCCGGGACUUCGCCACCGUGCGGAAG |
| | | GUGCUGUCCAUGCCCCAGGUGAACAUCGUGAAGAAGACCGAGGU |
| | | GCAGACCGGCGGCUUCUCCAAGGAGUCCAUCCUGCCCAAGCGGAA |
| | | CUCCGACAAGCUGAUCGCCCGGAAGAAGGACUGGGACCCCAAGAA |
| | | GUACGGCGGCUUCGACUCCCCCACCGUGGCCUACUCCGUGCUGGU |
| | | GGUGGCCAAGGUGGAGAAGGGCAAGUCCAAGAAGCUGAAGUCCG |
| | | UGAAGGAGCUGCUGGGCAUCACCAUCAUGGAGCGGUCCUCCUUCG |
| | | AGAAGAACCCCAUCGACUUCCUGGAGGCCAAGGGCUACAAGGAG |
| | | GUGAAGAAGGACCUGAUCAUCAAGCUGCCCAAGUACUCCCUGUUC |
| | | GAGCUGGAGAACGGCCGGAAGCGGAUGCUGGCCUCCGCCGGCGAG |
| | | CUGCAGAAGGGCAACGAGCUGGCCCUGCCCUCCAAGUACGUGAAC |
| | | UUCCUGUACCUGGCCUCCCACUACGAGAAGCUGAAGGGCUCCCCC |
| | | GAGGACAACGAGCAGAAGCAGCUGUUCGUGGAGCAGCACAAGCA |
| | | CUACCUGGACGAGAUCAUCGAGCAGAUCUCCGAGUUCUCCAAGCG |
| | | GGUGAUCCUGGCCGACGCCAACCUGGACAAGGUGCUGUCCGCCUA |
| | | CAACAAGCACCGGGACAAGCCCAUCCGGGAGCAGGCCGAGAACAU |
| | | CAUCCACCUGUUCACCCUGACCAACCUGGGCGCCCCCGCCGCCUU |
| | | CAAGUACUUCGACACCACCAUCGACCGGAAGCGGUACACCUCCAC |
| | | CAAGGAGGUGCUGGACGCCACCCUGAUCCACCAGUCCAUCACCGG |
| | | CCUGUACGAGACCCGGAUCGACCUGUCCCAGCUGGGCGGCGACGG |
| | | CGGCGGCUCCCCCAAGAAGAAGCGGAAGGUGUGACUAGCACCAGC |
| | | CUCAAGAACACCCGAAUGGAGUCUCUAAGCUACAUAAUACCAACU |
| | | UACACUUUACAAAAUGUUGUCCCCCAAAAUGUAGCCAUUCGUAU |
| | | CUGCUCCUAAUAAAAAGAAAGUUUCUUCACAUUCUCUCGAGAAA |
| | | AAAAAAAAAUGGAAAAAAAAAAAACGGAAAAAAAAAAAAGGUAA |
| | | AAAAAAAAAAUAUAAAAAAAAAAAACAUAAAAAAAAAAAACGAA |
| | | AAAAAAAAAACGUAAAAAAAAAAAACUCAAAAAAAAAAAAGAUA |
| | | AAAAAAAAAAACCUAAAAAAAAAAAAUGUAAAAAAAAAAAAGGG |
| | | AAAAAAAAAAAACGCAAAAAAAAAAAACACAAAAAAAAAAAAUG |
| | | CAAAAAAAAAAAAUCGAAAAAAAAAAAAUCUAAAAAAAAAAAAC |
| | | GAAAAAAAAAAAACCCAAAAAAAAAAAAGACAAAAAAAAAAAAU |
| | | AGAAAAAAAAAAAAGUUAAAAAAAAAAAACUGAAAAAAAAAAAA |
| | | UUUAAAAAAAAAAAAUCUAG |
| 2 | Open reading | AUGGAGGCCUCCCCCGCCUCCGGCCCCCGGCACCUGAUGGACCCC |
| | frame for BC22n | CACAUCUUCACCUCCAACUUCAACAACGGCAUCGGCCGGCACAAG |
| | | ACCUACCUGUGCUACGAGGUGGAGCGGCUGGACAACGGCACCUCC |
| | | GUGAAGAUGGACCAGCACCGGGGCUUCCUGCACAACCAGGCCAAG |
| | | AACCUGCUGUGCGGCUUCUACGGCCGGCACGCCGAGCUGCGGUUC |
| | | CUGGACCUGGUGCCCUCCCUGCAGCUGGACCCCGCCCAGAUCUAC |
| | | CGGGUGACCUGGUUCAUCUCCUGGUCCCCCUGCUUCUCCUGGGGC |
| | | UGCGCCGGCGAGGUGCGGGCCUUCCUGCAGGAGAACACCCACGUG |
| | | CGGCUGCGGAUCUUCGCCGCCCGGAUCUACGACUACGACCCCCUG |
| | | UACAAGGAGGCCCUGCAGAUGCUGCGGGACGCCGGCGCCCAGGUG |
| | | UCCAUCAUGACCUACGACGAGUUCAAGCACUGCUGGGACACCUUC |
| | | GUGGACCACCAGGGCUGCCCCUUCCAGCCCUGGGACGGCCUGGAC |
| | | GAGCACUCCCAGGCCCUGUCCGGCCGGCUGCGGGCCAUCCUGCAG |
| | | AACCAGGGCAACUCCGGCUCCGAGACCCCCGGCACCUCCGAGUCC |
| | | GCCACCCCCGAGUCCGACAAGAAGUACUCCAUCGGCCUGGCCAUC |
| | | GGCACCAACUCCGUGGGCUGGGCCGUGAUCACCGACGAGUACAAG |
| | | GUGCCCUCCAAGAAGUUCAAGGUGCUGGGCAACACCGACCGGCAC |
| | | UCCAUCAAGAAGAACCUGAUCGGCGCCCUGCUGUUCGACUCCGGC |
| | | GAGACCGCCGAGGCCACCCGGCUGAAGCGGACCGCCCGGCGGCGG |
| | | UACACCCGGCGGAAGAACCGGAUCUGCUACCUGCAGGAGAUCUUC |
| | | UCCAACGAGAUGGCCAAGGUGGACGACUCCUUCUUCCACCGGCUG |
| | | GAGGAGUCCUUCCUGGUGGAGGAGGACAAGAAGCACGAGCGGCA |
| | | CCCCAUCUUCGGCAACAUCGUGGACGAGGUGGCCUACCACGAGAA |
| | | GUACCCCACCAUCUACCACCUGCGGAAGAAGCUGGUGGACUCCAC |
| | | CGACAAGGCCGACCUGCGGCUGAUCUACCUGGCCCUGGCCCACAU |
| | | GAUCAAGUUCCGGGGCCACUUCCUGAUCGAGGGCGACCUGAACCC |
| | | CGACAACUCCGACGUGGACAAGCUGUUCAUCCAGCUGGUGCAGAC |
| | | CUACAACCAGCUGUUCGAGGAGAACCCCAUCAACGCCUCCGGCGU |
| | | GGACGCCAAGGCCAUCCUGUCCGCCCGGCUGUCCAAGUCCCGGCG |
| | | GCUGGAGAACCUGAUCGCCCAGCUGCCCGGCGAGAAGAAGAACGG |
| | | CCUGUUCGGCAACCUGAUCGCCCUGUCCCUGGGCCUGACCCCCAA |
| | | CUUCAAGUCCAACUUCGACCUGGCCGAGGACGCCAAGCUGCAGCU |
| | | GUCCAAGGACACCUACGACGACGACCUGGACAACCUGCUGGCCCA |
| | | GAUCGGCGACCAGUACGCCGACCUGUUCCUGGCCGCCAAGAACCU |
| | | GUCCGACGCCAUCCUGCUGUCCGACAUCCUGCGGGUGAACACCGA |
| | | GAUCACCAAGGCCCCCCUGUCCGCCUCCAUGAUCAAGCGGUACGA |
| | | CGAGCACCACCAGGACCUGACCCUGCUGAAGGCCCUGGUGCGGCA |
| | | GCAGCUGCCCGAGAAGUACAAGGAGAUCUUCUUCGACCAGUCCAA |
| | | GAACGGCUACGCCGGCUACAUCGACGGCGGCGCCUCCCAGGAGGA |
| | | GUUCUACAAGUUCAUCAAGCCCAUCCUGGAGAAGAUGGACGGCA |
| | | CCGAGGAGCUGCUGGUGAAGCUGAACCGGGAGGACCUGCUGCGG |
| | | AAGCAGCGGACCUUCGACAACGGCUCCAUCCCCCACCAGAUCCAC |
| | | CUGGGCGAGCUGCACGCCAUCCUGCGGCGGCAGGAGGACUUCUAC |
| | | CCCUUCCUGAAGGACAACCGGGAGAAGAUCGAGAAGAUCCUGACC |
| | | UUCCGGAUCCCCUACUACGUGGGCCCCCUGGCCCGGGGCAACUCC |
| | | CGGUUCGCCUGGAUGACCCGGAAGUCCGAGGAGACCAUCACCCCC |
| | | UGGAACUUCGAGGAGGUGGUGGACAAGGGCGCCUCCGCCCAGUCC |
| | | UUCAUCGAGCGGAUGACCAACUUCGACAAGAACCUGCCCAACGAG |
| | | AAGGUGCUGCCCAAGCACUCCCUGCUGUACGAGUACUUCACCGUG |
| | | UACAACGAGCUGACCAAGGUGAAGUACGUGACCGAGGGCAUGCG |
| | | GAAGCCCGCCUUCCUGUCCGGCGAGCAGAAGAAGGCCAUCGUGGA |
| | | CCUGCUGUUCAAGACCAACCGGAAGGUGACCGUGAAGCAGCUGA |
| | | AGGAGGACUACUUCAAGAAGAUCGAGUGCUUCGACUCCGUGGAG |
| | | AUCUCCGGCGUGGAGGACCGGUUCAACGCCUCCCUGGGCACCUAC |
| | | CACGACCUGCUGAAGAUCAUCAAGGACAAGGACUUCCUGGACAAC |
| | | GAGGAGAACGAGGACAUCCUGGAGGACAUCGUGCUGACCCUGAC |
| | | CCUGUUCGAGGACCGGGAGAUGAUCGAGGAGCGGCUGAAGACCU |
| | | ACGCCCACCUGUUCGACGACAAGGUGAUGAAGCAGCUGAAGCGGC |
| | | GGCGGUACACCGGCUGGGGCCGGCUGUCCCGGAAGCUGAUCAACG |
| | | GCAUCCGGGACAAGCAGUCCGGCAAGACCAUCCUGGACUUCCUGA |
| | | AGUCCGACGGCUUCGCCAACCGGAACUUCAUGCAGCUGAUCCACG |
| | | ACGACUCCCUGACCUUCAAGGAGGACAUCCAGAAGGCCCAGGUGU |
| | | CCGGCCAGGGCGACUCCCUGCACGAGCACAUCGCCAACCUGGCCG |
| | | GCUCCCCCGCCAUCAAGAAGGGCAUCCUGCAGACCGUGAAGGUGG |
| | | UGGACGAGCUGGUGAAGGUGAUGGGCCGGCACAAGCCCGAGAAC |
| | | AUCGUGAUCGAGAUGGCCCGGGAGAACCAGACCACCCAGAAGGGC |
| | | CAGAAGAACUCCCGGGAGCGGAUGAAGCGGAUCGAGGAGGGCAU |
| | | CAAGGAGCUGGGCUCCCAGAUCCUGAAGGAGCACCCCGUGGAGAA |
| | | CACCCAGCUGCAGAACGAGAAGCUGUACCUGUACUACCUGCAGAA |
| | | CGGCCGGGACAUGUACGUGGACCAGGAGCUGGACAUCAACCGGCU |
| | | GUCCGACUACGACGUGGACCACAUCGUGCCCCAGUCCUUCCUGAA |
| | | GGACGACUCCAUCGACAACAAGGUGCUGACCCGGUCCGACAAGAA |
| | | CCGGGGCAAGUCCGACAACGUGCCCUCCGAGGAGGUGGUGAAGA |
| | | AGAUGAAGAACUACUGGCGGCAGCUGCUGAACGCCAAGCUGAUC |
| | | ACCCAGCGGAAGUUCGACAACCUGACCAAGGCCGAGCGGGGCGGC |
| | | CUGUCCGAGCUGGACAAGGCCGGCUUCAUCAAGCGGCAGCUGGUG |
| | | GAGACCCGGCAGAUCACCAAGCACGUGGCCCAGAUCCUGGACUCC |
| | | CGGAUGAACACCAAGUACGACGAGAACGACAAGCUGAUCCGGGA |
| | | GGUGAAGGUGAUCACCCUGAAGUCCAAGCUGGUGUCCGACUUCC |
| | | GGAAGGACUUCCAGUUCUACAAGGUGCGGGAGAUCAACAACUAC |
| | | CACCACGCCCACGACGCCUACCUGAACGCCGUGGUGGGCACCGCC |
| | | CUGAUCAAGAAGUACCCCAAGCUGGAGUCCGAGUUCGUGUACGG |
| | | CGACUACAAGGUGUACGACGUGCGGAAGAUGAUCGCCAAGUCCG |
| | | AGCAGGAGAUCGGCAAGGCCACCGCCAAGUACUUCUUCUACUCCA |
| | | ACAUCAUGAACUUCUUCAAGACCGAGAUCACCCUGGCCAACGGCG |
| | | AGAUCCGGAAGCGGCCCCUGAUCGAGACCAACGGCGAGACCGGCG |
| | | AGAUCGUGUGGGACAAGGGCCGGGACUUCGCCACCGUGCGGAAG |
| | | GUGCUGUCCAUGCCCCAGGUGAACAUCGUGAAGAAGACCGAGGU |
| | | GCAGACCGGCGGCUUCUCCAAGGAGUCCAUCCUGCCCAAGCGGAA |
| | | CUCCGACAAGCUGAUCGCCCGGAAGAAGGACUGGGACCCCAAGAA |
| | | GUACGGCGGCUUCGACUCCCCCACCGUGGCCUACUCCGUGCUGGU |
| | | GGUGGCCAAGGUGGAGAAGGGCAAGUCCAAGAAGCUGAAGUCCG |
| | | UGAAGGAGCUGCUGGGCAUCACCAUCAUGGAGCGGUCCUCCUUCG |
| | | AGAAGAACCCCAUCGACUUCCUGGAGGCCAAGGGCUACAAGGAG |
| | | GUGAAGAAGGACCUGAUCAUCAAGCUGCCCAAGUACUCCCUGUUC |
| | | GAGCUGGAGAACGGCCGGAAGCGGAUGCUGGCCUCCGCCGGCGAG |
| | | CUGCAGAAGGGCAACGAGCUGGCCCUGCCCUCCAAGUACGUGAAC |
| | | UUCCUGUACCUGGCCUCCCACUACGAGAAGCUGAAGGGCUCCCCC |
| | | GAGGACAACGAGCAGAAGCAGCUGUUCGUGGAGCAGCACAAGCA |
| | | CUACCUGGACGAGAUCAUCGAGCAGAUCUCCGAGUUCUCCAAGCG |
| | | GGUGAUCCUGGCCGACGCCAACCUGGACAAGGUGCUGUCCGCCUA |
| | | CAACAAGCACCGGGACAAGCCCAUCCGGGAGCAGGCCGAGAACAU |
| | | CAUCCACCUGUUCACCCUGACCAACCUGGGCGCCCCCGCCGCCUU |
| | | CAAGUACUUCGACACCACCAUCGACCGGAAGCGGUACACCUCCAC |
| | | CAAGGAGGUGCUGGACGCCACCCUGAUCCACCAGUCCAUCACCGG |
| | | CCUGUACGAGACCCGGAUCGACCUGUCCCAGCUGGGCGGCGACGG |
| | | CGGCGGCUCCCCCAAGAAGAAGCGGAAGGUGUGA |
| 3 | Amino acid | MEASPASGPRHLMDPHIFTSNFNNGIGRHKTYLCYEVERLDNGTSVKM |
| | sequence for | DQHRGFLHNQAKNLLCGFYGRHAELRFLDLVPSLQLDPAQIYRVTWFI |
| | BC22n | SWSPCFSWGCAGEVRAFLQENTHVRLRIFAARIYDYDPLYKEALQMLR |
| | | DAGAQVSIMTYDEFKHCWDTFVDHQGCPFQPWDGLDEHSQALSGRL |
| | | RAILQNQGNSGSETPGTSESATPESDKKYSIGLAIGTNSVGWAVITDEY |
| | | KVPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGNIV |
| | | DEVAYHEKYPTIYHLRKKLVDSTDKADLRLIYLALAHMIKFRGHFLIE |
| | | GDLNPDNSDVDKLFIQLVQTYNQLFEENPINASGVDAKAILSARLSKSR |
| | | RLENLIAQLPGEKKNGLFGNLIALSLGLTPNFKSNFDLAEDAKLQLSKD |
| | | TYDDDLDNLLAQIGDQYADLFLAAKNLSDAILLSDILRVNTEITKAPLS |
| | | ASMIKRYDEHHQDLTLLKALVRQQLPEKYKEIFFDQSKNGYAGYIDGG |
| | | ASQEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAWM |
| | | TRKSEETITPWNFEEVVDKGASAQSFIERMTNFDKNLPNEKVLPKHSLL |
| | | YEYFTVYNELTKVKYVTEGMRKPAFLSGEQKKAIVDLLFKTNRKVTV |
| | | KQLKEDYFKKIECFDSVEISGVEDRFNASLGTYHDLLKIIKDKDFLDNE |
| | | ENEDILEDIVLTLTLFEDREMIEERLKTYAHLFDDKVMKQLKRRRYTG |
| | | WGRLSRKLINGIRDKQSGKTILDFLKSDGFANRNFMQLIHDDSLTFKED |
| | | IQKAQVSGQGDSLHEHIANLAGSPAIKKGILQTVKVVDELVKVMGRHK |
| | | PENIVIEMARENQTTQKGQKNSRERMKRIEEGIKELGSQILKEHPVENT |
| | | QLQNEKLYLYYLQNGRDMYVDQELDINRLSDYDVDHIVPQSFLKDDSI |
| | | DNKVLTRSDKNRGKSDNVPSEEVVKKMKNYWRQLLNAKLITQRKFD |
| | | NLTKAERGGLSELDKAGFIKRQLVETRQITKHVAQILDSRMNTKYDEN |
| | | DKLIREVKVITLKSKLVSDFRKDFQFYKVREINNYHHAHDAYLNAVVG |
| | | TALIKKYPKLESEFVYGDYKVYDVRKMIAKSEQEIGKATAKYFFYSNI |
| | | MNFFKTEITLANGEIRKRPLIETNGETGEIVWDKGRDFATVRKVLSMPQ |
| | | VNIVKKTEVQTGGFSKESILPKRNSDKLIARKKDWDPKKYGGFDSPTV |
| | | AYSVLVVAKVEKGKSKKLKSVKELLGITIMERSSFEKNPIDFLEAKGYK |
| | | EVKKDLIIKLPKYSLFELENGRKRMLASAGELQKGNELALPSKYVNFL |
| | | YLASHYEKLKGSPEDNEQKQLFVEQHKHYLDEIIEQISEFSKRVILADA |
| | | NLDKVLSAYNKHRDKPIREQAENIIHLFTLTNLGAPAAFKYFDTTIDRK |
| | | RYTSTKEVLDATLIHQSITGLYETRIDLSQLGGDGGGSPKKKRKV |
| 4 | mRNA encoding | GGGAAGCUCAGAAUAAACGCUCAACUUUGGCCGGAUCUGCCACCA |
| | BC22n with Hibit | UGGAGGCCUCCCCCGCCUCCGGCCCCCGGCACCUGAUGGACCCCC |
| | tag | ACAUCUUCACCUCCAACUUCAACAACGGCAUCGGCCGGCACAAGA |
| | | CCUACCUGUGCUACGAGGUGGAGCGGCUGGACAACGGCACCUCCG |
| | | UGAAGAUGGACCAGCACCGGGGCUUCCUGCACAACCAGGCCAAGA |
| | | ACCUGCUGUGCGGCUUCUACGGCCGGCACGCCGAGCUGCGGUUCC |
| | | UGGACCUGGUGCCCUCCCUGCAGCUGGACCCCGCCCAGAUCUACC |
| | | GGGUGACCUGGUUCAUCUCCUGGUCCCCCUGCUUCUCCUGGGGCU |
| | | GCGCCGGCGAGGUGCGGGCCUUCCUGCAGGAGAACACCCACGUGC |
| | | GGCUGCGGAUCUUCGCCGCCCGGAUCUACGACUACGACCCCCUGU |
| | | ACAAGGAGGCCCUGCAGAUGCUGCGGGACGCCGGCGCCCAGGUGU |
| | | CCAUCAUGACCUACGACGAGUUCAAGCACUGCUGGGACACCUUCG |
| | | UGGACCACCAGGGCUGCCCCUUCCAGCCCUGGGACGGCCUGGACG |
| | | AGCACUCCCAGGCCCUGUCCGGCCGGCUGCGGGCCAUCCUGCAGA |
| | | ACCAGGGCAACUCCGGCUCCGAGACCCCCGGCACCUCCGAGUCCG |
| | | CCACCCCCGAGUCCGACAAGAAGUACUCCAUCGGCCUGGCCAUCG |
| | | GCACCAACUCCGUGGGCUGGGCCGUGAUCACCGACGAGUACAAGG |
| | | UGCCCUCCAAGAAGUUCAAGGUGCUGGGCAACACCGACCGGCACU |
| | | CCAUCAAGAAGAACCUGAUCGGCGCCCUGCUGUUCGACUCCGGCG |
| | | AGACCGCCGAGGCCACCCGGCUGAAGCGGACCGCCCGGCGGCGGU |
| | | ACACCCGGCGGAAGAACCGGAUCUGCUACCUGCAGGAGAUCUUCU |
| | | CCAACGAGAUGGCCAAGGUGGACGACUCCUUCUUCCACCGGCUGG |
| | | AGGAGUCCUUCCUGGUGGAGGAGGACAAGAAGCACGAGCGGCAC |
| | | CCCAUCUUCGGCAACAUCGUGGACGAGGUGGCCUACCACGAGAAG |
| | | UACCCCACCAUCUACCACCUGCGGAAGAAGCUGGUGGACUCCACC |
| | | GACAAGGCCGACCUGCGGCUGAUCUACCUGGCCCUGGCCCACAUG |
| | | AUCAAGUUCCGGGGCCACUUCCUGAUCGAGGGCGACCUGAACCCC |
| | | GACAACUCCGACGUGGACAAGCUGUUCAUCCAGCUGGUGCAGACC |
| | | UACAACCAGCUGUUCGAGGAGAACCCCAUCAACGCCUCCGGCGUG |
| | | GACGCCAAGGCCAUCCUGUCCGCCCGGCUGUCCAAGUCCCGGCGG |
| | | CUGGAGAACCUGAUCGCCCAGCUGCCCGGCGAGAAGAAGAACGGC |
| | | CUGUUCGGCAACCUGAUCGCCCUGUCCCUGGGCCUGACCCCCAAC |
| | | UUCAAGUCCAACUUCGACCUGGCCGAGGACGCCAAGCUGCAGCUG |
| | | UCCAAGGACACCUACGACGACGACCUGGACAACCUGCUGGCCCAG |
| | | AUCGGCGACCAGUACGCCGACCUGUUCCUGGCCGCCAAGAACCUG |
| | | UCCGACGCCAUCCUGCUGUCCGACAUCCUGCGGGUGAACACCGAG |
| | | AUCACCAAGGCCCCCCUGUCCGCCUCCAUGAUCAAGCGGUACGAC |
| | | GAGCACCACCAGGACCUGACCCUGCUGAAGGCCCUGGUGCGGCAG |
| | | CAGCUGCCCGAGAAGUACAAGGAGAUCUUCUUCGACCAGUCCAAG |
| | | AACGGCUACGCCGGCUACAUCGACGGCGGCGCCUCCCAGGAGGAG |
| | | UUCUACAAGUUCAUCAAGCCCAUCCUGGAGAAGAUGGACGGCACC |
| | | GAGGAGCUGCUGGUGAAGCUGAACCGGGAGGACCUGCUGCGGAA |
| | | GCAGCGGACCUUCGACAACGGCUCCAUCCCCCACCAGAUCCACCU |
| | | GGGCGAGCUGCACGCCAUCCUGCGGCGGCAGGAGGACUUCUACCC |
| | | CUUCCUGAAGGACAACCGGGAGAAGAUCGAGAAGAUCCUGACCU |
| | | UCCGGAUCCCCUACUACGUGGGCCCCCUGGCCCGGGGCAACUCCC |
| | | GGUUCGCCUGGAUGACCCGGAAGUCCGAGGAGACCAUCACCCCCU |
| | | GGAACUUCGAGGAGGUGGUGGACAAGGGCGCCUCCGCCCAGUCCU |
| | | UCAUCGAGCGGAUGACCAACUUCGACAAGAACCUGCCCAACGAGA |
| | | AGGUGCUGCCCAAGCACUCCCUGCUGUACGAGUACUUCACCGUGU |
| | | ACAACGAGCUGACCAAGGUGAAGUACGUGACCGAGGGCAUGCGG |
| | | AAGCCCGCCUUCCUGUCCGGCGAGCAGAAGAAGGCCAUCGUGGAC |
| | | CUGCUGUUCAAGACCAACCGGAAGGUGACCGUGAAGCAGCUGAA |
| | | GGAGGACUACUUCAAGAAGAUCGAGUGCUUCGACUCCGUGGAGA |
| | | UCUCCGGCGUGGAGGACCGGUUCAACGCCUCCCUGGGCACCUACC |
| | | ACGACCUGCUGAAGAUCAUCAAGGACAAGGACUUCCUGGACAAC |
| | | GAGGAGAACGAGGACAUCCUGGAGGACAUCGUGCUGACCCUGAC |
| | | CCUGUUCGAGGACCGGGAGAUGAUCGAGGAGCGGCUGAAGACCU |
| | | ACGCCCACCUGUUCGACGACAAGGUGAUGAAGCAGCUGAAGCGGC |
| | | GGCGGUACACCGGCUGGGGCCGGCUGUCCCGGAAGCUGAUCAACG |
| | | GCAUCCGGGACAAGCAGUCCGGCAAGACCAUCCUGGACUUCCUGA |
| | | AGUCCGACGGCUUCGCCAACCGGAACUUCAUGCAGCUGAUCCACG |
| | | ACGACUCCCUGACCUUCAAGGAGGACAUCCAGAAGGCCCAGGUGU |
| | | CCGGCCAGGGCGACUCCCUGCACGAGCACAUCGCCAACCUGGCCG |
| | | GCUCCCCCGCCAUCAAGAAGGGCAUCCUGCAGACCGUGAAGGUGG |
| | | UGGACGAGCUGGUGAAGGUGAUGGGCCGGCACAAGCCCGAGAAC |
| | | AUCGUGAUCGAGAUGGCCCGGGAGAACCAGACCACCCAGAAGGGC |
| | | CAGAAGAACUCCCGGGAGCGGAUGAAGCGGAUCGAGGAGGGCAU |
| | | CAAGGAGCUGGGCUCCCAGAUCCUGAAGGAGCACCCCGUGGAGAA |
| | | CACCCAGCUGCAGAACGAGAAGCUGUACCUGUACUACCUGCAGAA |
| | | CGGCCGGGACAUGUACGUGGACCAGGAGCUGGACAUCAACCGGCU |
| | | GUCCGACUACGACGUGGACCACAUCGUGCCCCAGUCCUUCCUGAA |
| | | GGACGACUCCAUCGACAACAAGGUGCUGACCCGGUCCGACAAGAA |
| | | CCGGGGCAAGUCCGACAACGUGCCCUCCGAGGAGGUGGUGAAGA |
| | | AGAUGAAGAACUACUGGCGGCAGCUGCUGAACGCCAAGCUGAUC |
| | | ACCCAGCGGAAGUUCGACAACCUGACCAAGGCCGAGCGGGGCGGC |
| | | CUGUCCGAGCUGGACAAGGCCGGCUUCAUCAAGCGGCAGCUGGUG |
| | | GAGACCCGGCAGAUCACCAAGCACGUGGCCCAGAUCCUGGACUCC |
| | | CGGAUGAACACCAAGUACGACGAGAACGACAAGCUGAUCCGGGA |
| | | GGUGAAGGUGAUCACCCUGAAGUCCAAGCUGGUGUCCGACUUCC |
| | | GGAAGGACUUCCAGUUCUACAAGGUGCGGGAGAUCAACAACUAC |
| | | CACCACGCCCACGACGCCUACCUGAACGCCGUGGUGGGCACCGCC |
| | | CUGAUCAAGAAGUACCCCAAGCUGGAGUCCGAGUUCGUGUACGG |
| | | CGACUACAAGGUGUACGACGUGCGGAAGAUGAUCGCCAAGUCCG |
| | | AGCAGGAGAUCGGCAAGGCCACCGCCAAGUACUUCUUCUACUCCA |
| | | ACAUCAUGAACUUCUUCAAGACCGAGAUCACCCUGGCCAACGGCG |
| | | AGAUCCGGAAGCGGCCCCUGAUCGAGACCAACGGCGAGACCGGCG |
| | | AGAUCGUGUGGGACAAGGGCCGGGACUUCGCCACCGUGCGGAAG |
| | | GUGCUGUCCAUGCCCCAGGUGAACAUCGUGAAGAAGACCGAGGU |
| | | GCAGACCGGCGGCUUCUCCAAGGAGUCCAUCCUGCCCAAGCGGAA |
| | | CUCCGACAAGCUGAUCGCCCGGAAGAAGGACUGGGACCCCAAGAA |
| | | GUACGGCGGCUUCGACUCCCCCACCGUGGCCUACUCCGUGCUGGU |
| | | GGUGGCCAAGGUGGAGAAGGGCAAGUCCAAGAAGCUGAAGUCCG |
| | | UGAAGGAGCUGCUGGGCAUCACCAUCAUGGAGCGGUCCUCCUUCG |
| | | AGAAGAACCCCAUCGACUUCCUGGAGGCCAAGGGCUACAAGGAG |
| | | GUGAAGAAGGACCUGAUCAUCAAGCUGCCCAAGUACUCCCUGUUC |
| | | GAGCUGGAGAACGGCCGGAAGCGGAUGCUGGCCUCCGCCGGCGAG |
| | | CUGCAGAAGGGCAACGAGCUGGCCCUGCCCUCCAAGUACGUGAAC |
| | | UUCCUGUACCUGGCCUCCCACUACGAGAAGCUGAAGGGCUCCCCC |
| | | GAGGACAACGAGCAGAAGCAGCUGUUCGUGGAGCAGCACAAGCA |
| | | CUACCUGGACGAGAUCAUCGAGCAGAUCUCCGAGUUCUCCAAGCG |
| | | GGUGAUCCUGGCCGACGCCAACCUGGACAAGGUGCUGUCCGCCUA |
| | | CAACAAGCACCGGGACAAGCCCAUCCGGGAGCAGGCCGAGAACAU |
| | | CAUCCACCUGUUCACCCUGACCAACCUGGGCGCCCCCGCCGCCUU |
| | | CAAGUACUUCGACACCACCAUCGACCGGAAGCGGUACACCUCCAC |
| | | CAAGGAGGUGCUGGACGCCACCCUGAUCCACCAGUCCAUCACCGG |
| | | CCUGUACGAGACCCGGAUCGACCUGUCCCAGCUGGGCGGCGACGG |
| | | CGGCGGCUCCCCCAAGAAGAAGCGGAAGGUGUCCGAGUCCGCCAC |
| | | CCCCGAGUCCGUGUCCGGCUGGCGGCUGUUCAAGAAGAUCUCCUG |
| | | ACUAGCACCAGCCUCAAGAACACCCGAAUGGAGUCUCUAAGCUAC |
| | | AUAAUACCAACUUACACUUUACAAAAUGUUGUCCCCCAAAAUGU |
| | | AGCCAUUCGUAUCUGCUCCUAAUAAAAAGAAAGUUUCUUCACAU |
| | | UCUCUCGAGAAAAAAAAAAAAUGGAAAAAAAAAAAACGGAAAAA |
| | | AAAAAAAGGUAAAAAAAAAAAAUAUAAAAAAAAAAAACAUAAAA |
| | | AAAAAAAACGAAAAAAAAAAAACGUAAAAAAAAAAAACUCAAAA |
| | | AAAAAAAAGAUAAAAAAAAAAAACCUAAAAAAAAAAAAUGUAAA |
| | | AAAAAAAAAGGGAAAAAAAAAAAACGCAAAAAAAAAAAACACAA |
| | | AAAAAAAAAAUGCAAAAAAAAAAAAUCGAAAAAAAAAAAAUCUA |
| | | AAAAAAAAAAACGAAAAAAAAAAAACCCAAAAAAAAAAAAGACA |
| | | AAAAAAAAAAAUAGAAAAAAAAAAAAGUUAAAAAAAAAAAACUG |
| | | AAAAAAAAAAAAUUUAAAAAAAAAAAAUCUAG |
| 5 | Open reading | AUGGAGGCCUCCCCCGCCUCCGGCCCCCGGCACCUGAUGGACCCC |
| | frame for BC22n | CACAUCUUCACCUCCAACUUCAACAACGGCAUCGGCCGGCACAAG |
| | with Hibit tag | ACCUACCUGUGCUACGAGGUGGAGCGGCUGGACAACGGCACCUCC |
| | | GUGAAGAUGGACCAGCACCGGGGCUUCCUGCACAACCAGGCCAAG |
| | | AACCUGCUGUGCGGCUUCUACGGCCGGCACGCCGAGCUGCGGUUC |
| | | CUGGACCUGGUGCCCUCCCUGCAGCUGGACCCCGCCCAGAUCUAC |
| | | CGGGUGACCUGGUUCAUCUCCUGGUCCCCCUGCUUCUCCUGGGGC |
| | | UGCGCCGGCGAGGUGCGGGCCUUCCUGCAGGAGAACACCCACGUG |
| | | CGGCUGCGGAUCUUCGCCGCCCGGAUCUACGACUACGACCCCCUG |
| | | UACAAGGAGGCCCUGCAGAUGCUGCGGGACGCCGGCGCCCAGGUG |
| | | UCCAUCAUGACCUACGACGAGUUCAAGCACUGCUGGGACACCUUC |
| | | GUGGACCACCAGGGCUGCCCCUUCCAGCCCUGGGACGGCCUGGAC |
| | | GAGCACUCCCAGGCCCUGUCCGGCCGGCUGCGGGCCAUCCUGCAG |
| | | AACCAGGGCAACUCCGGCUCCGAGACCCCCGGCACCUCCGAGUCC |
| | | GCCACCCCCGAGUCCGACAAGAAGUACUCCAUCGGCCUGGCCAUC |
| | | GGCACCAACUCCGUGGGCUGGGCCGUGAUCACCGACGAGUACAAG |
| | | GUGCCCUCCAAGAAGUUCAAGGUGCUGGGCAACACCGACCGGCAC |
| | | UCCAUCAAGAAGAACCUGAUCGGCGCCCUGCUGUUCGACUCCGGC |
| | | GAGACCGCCGAGGCCACCCGGCUGAAGCGGACCGCCCGGCGGCGG |
| | | UACACCCGGCGGAAGAACCGGAUCUGCUACCUGCAGGAGAUCUUC |
| | | UCCAACGAGAUGGCCAAGGUGGACGACUCCUUCUUCCACCGGCUG |
| | | GAGGAGUCCUUCCUGGUGGAGGAGGACAAGAAGCACGAGCGGCA |
| | | CCCCAUCUUCGGCAACAUCGUGGACGAGGUGGCCUACCACGAGAA |
| | | GUACCCCACCAUCUACCACCUGCGGAAGAAGCUGGUGGACUCCAC |
| | | CGACAAGGCCGACCUGCGGCUGAUCUACCUGGCCCUGGCCCACAU |
| | | GAUCAAGUUCCGGGGCCACUUCCUGAUCGAGGGCGACCUGAACCC |
| | | CGACAACUCCGACGUGGACAAGCUGUUCAUCCAGCUGGUGCAGAC |
| | | CUACAACCAGCUGUUCGAGGAGAACCCCAUCAACGCCUCCGGCGU |
| | | GGACGCCAAGGCCAUCCUGUCCGCCCGGCUGUCCAAGUCCCGGCG |
| | | GCUGGAGAACCUGAUCGCCCAGCUGCCCGGCGAGAAGAAGAACGG |
| | | CCUGUUCGGCAACCUGAUCGCCCUGUCCCUGGGCCUGACCCCCAA |
| | | CUUCAAGUCCAACUUCGACCUGGCCGAGGACGCCAAGCUGCAGCU |
| | | GUCCAAGGACACCUACGACGACGACCUGGACAACCUGCUGGCCCA |
| | | GAUCGGCGACCAGUACGCCGACCUGUUCCUGGCCGCCAAGAACCU |
| | | GUCCGACGCCAUCCUGCUGUCCGACAUCCUGCGGGUGAACACCGA |
| | | GAUCACCAAGGCCCCCCUGUCCGCCUCCAUGAUCAAGCGGUACGA |
| | | CGAGCACCACCAGGACCUGACCCUGCUGAAGGCCCUGGUGCGGCA |
| | | GCAGCUGCCCGAGAAGUACAAGGAGAUCUUCUUCGACCAGUCCAA |
| | | GAACGGCUACGCCGGCUACAUCGACGGCGGCGCCUCCCAGGAGGA |
| | | GUUCUACAAGUUCAUCAAGCCCAUCCUGGAGAAGAUGGACGGCA |
| | | CCGAGGAGCUGCUGGUGAAGCUGAACCGGGAGGACCUGCUGCGG |
| | | AAGCAGCGGACCUUCGACAACGGCUCCAUCCCCCACCAGAUCCAC |
| | | CUGGGCGAGCUGCACGCCAUCCUGCGGCGGCAGGAGGACUUCUAC |
| | | CCCUUCCUGAAGGACAACCGGGAGAAGAUCGAGAAGAUCCUGACC |
| | | UUCCGGAUCCCCUACUACGUGGGCCCCCUGGCCCGGGGCAACUCC |
| | | CGGUUCGCCUGGAUGACCCGGAAGUCCGAGGAGACCAUCACCCCC |
| | | UGGAACUUCGAGGAGGUGGUGGACAAGGGCGCCUCCGCCCAGUCC |
| | | UUCAUCGAGCGGAUGACCAACUUCGACAAGAACCUGCCCAACGAG |
| | | AAGGUGCUGCCCAAGCACUCCCUGCUGUACGAGUACUUCACCGUG |
| | | UACAACGAGCUGACCAAGGUGAAGUACGUGACCGAGGGCAUGCG |
| | | GAAGCCCGCCUUCCUGUCCGGCGAGCAGAAGAAGGCCAUCGUGGA |
| | | CCUGCUGUUCAAGACCAACCGGAAGGUGACCGUGAAGCAGCUGA |
| | | AGGAGGACUACUUCAAGAAGAUCGAGUGCUUCGACUCCGUGGAG |
| | | AUCUCCGGCGUGGAGGACCGGUUCAACGCCUCCCUGGGCACCUAC |
| | | CACGACCUGCUGAAGAUCAUCAAGGACAAGGACUUCCUGGACAAC |
| | | GAGGAGAACGAGGACAUCCUGGAGGACAUCGUGCUGACCCUGAC |
| | | CCUGUUCGAGGACCGGGAGAUGAUCGAGGAGCGGCUGAAGACCU |
| | | ACGCCCACCUGUUCGACGACAAGGUGAUGAAGCAGCUGAAGCGGC |
| | | GGCGGUACACCGGCUGGGGCCGGCUGUCCCGGAAGCUGAUCAACG |
| | | GCAUCCGGGACAAGCAGUCCGGCAAGACCAUCCUGGACUUCCUGA |
| | | AGUCCGACGGCUUCGCCAACCGGAACUUCAUGCAGCUGAUCCACG |
| | | ACGACUCCCUGACCUUCAAGGAGGACAUCCAGAAGGCCCAGGUGU |
| | | CCGGCCAGGGCGACUCCCUGCACGAGCACAUCGCCAACCUGGCCG |
| | | GCUCCCCCGCCAUCAAGAAGGGCAUCCUGCAGACCGUGAAGGUGG |
| | | UGGACGAGCUGGUGAAGGUGAUGGGCCGGCACAAGCCCGAGAAC |
| | | AUCGUGAUCGAGAUGGCCCGGGAGAACCAGACCACCCAGAAGGGC |
| | | CAGAAGAACUCCCGGGAGCGGAUGAAGCGGAUCGAGGAGGGCAU |
| | | CAAGGAGCUGGGCUCCCAGAUCCUGAAGGAGCACCCCGUGGAGAA |
| | | CACCCAGCUGCAGAACGAGAAGCUGUACCUGUACUACCUGCAGAA |
| | | CGGCCGGGACAUGUACGUGGACCAGGAGCUGGACAUCAACCGGCU |
| | | GUCCGACUACGACGUGGACCACAUCGUGCCCCAGUCCUUCCUGAA |
| | | GGACGACUCCAUCGACAACAAGGUGCUGACCCGGUCCGACAAGAA |
| | | CCGGGGCAAGUCCGACAACGUGCCCUCCGAGGAGGUGGUGAAGA |
| | | AGAUGAAGAACUACUGGCGGCAGCUGCUGAACGCCAAGCUGAUC |
| | | ACCCAGCGGAAGUUCGACAACCUGACCAAGGCCGAGCGGGGCGGC |
| | | CUGUCCGAGCUGGACAAGGCCGGCUUCAUCAAGCGGCAGCUGGUG |
| | | GAGACCCGGCAGAUCACCAAGCACGUGGCCCAGAUCCUGGACUCC |
| | | CGGAUGAACACCAAGUACGACGAGAACGACAAGCUGAUCCGGGA |
| | | GGUGAAGGUGAUCACCCUGAAGUCCAAGCUGGUGUCCGACUUCC |
| | | GGAAGGACUUCCAGUUCUACAAGGUGCGGGAGAUCAACAACUAC |
| | | CACCACGCCCACGACGCCUACCUGAACGCCGUGGUGGGCACCGCC |
| | | CUGAUCAAGAAGUACCCCAAGCUGGAGUCCGAGUUCGUGUACGG |
| | | CGACUACAAGGUGUACGACGUGCGGAAGAUGAUCGCCAAGUCCG |
| | | AGCAGGAGAUCGGCAAGGCCACCGCCAAGUACUUCUUCUACUCCA |
| | | ACAUCAUGAACUUCUUCAAGACCGAGAUCACCCUGGCCAACGGCG |
| | | AGAUCCGGAAGCGGCCCCUGAUCGAGACCAACGGCGAGACCGGCG |
| | | AGAUCGUGUGGGACAAGGGCCGGGACUUCGCCACCGUGCGGAAG |
| | | GUGCUGUCCAUGCCCCAGGUGAACAUCGUGAAGAAGACCGAGGU |
| | | GCAGACCGGCGGCUUCUCCAAGGAGUCCAUCCUGCCCAAGCGGAA |
| | | CUCCGACAAGCUGAUCGCCCGGAAGAAGGACUGGGACCCCAAGAA |
| | | GUACGGCGGCUUCGACUCCCCCACCGUGGCCUACUCCGUGCUGGU |
| | | GGUGGCCAAGGUGGAGAAGGGCAAGUCCAAGAAGCUGAAGUCCG |
| | | UGAAGGAGCUGCUGGGCAUCACCAUCAUGGAGCGGUCCUCCUUCG |
| | | AGAAGAACCCCAUCGACUUCCUGGAGGCCAAGGGCUACAAGGAG |
| | | GUGAAGAAGGACCUGAUCAUCAAGCUGCCCAAGUACUCCCUGUUC |
| | | GAGCUGGAGAACGGCCGGAAGCGGAUGCUGGCCUCCGCCGGCGAG |
| | | CUGCAGAAGGGCAACGAGCUGGCCCUGCCCUCCAAGUACGUGAAC |
| | | UUCCUGUACCUGGCCUCCCACUACGAGAAGCUGAAGGGCUCCCCC |
| | | GAGGACAACGAGCAGAAGCAGCUGUUCGUGGAGCAGCACAAGCA |
| | | CUACCUGGACGAGAUCAUCGAGCAGAUCUCCGAGUUCUCCAAGCG |
| | | GGUGAUCCUGGCCGACGCCAACCUGGACAAGGUGCUGUCCGCCUA |
| | | CAACAAGCACCGGGACAAGCCCAUCCGGGAGCAGGCCGAGAACAU |
| | | CAUCCACCUGUUCACCCUGACCAACCUGGGCGCCCCCGCCGCCUU |
| | | CAAGUACUUCGACACCACCAUCGACCGGAAGCGGUACACCUCCAC |
| | | CAAGGAGGUGCUGGACGCCACCCUGAUCCACCAGUCCAUCACCGG |
| | | CCUGUACGAGACCCGGAUCGACCUGUCCCAGCUGGGCGGCGACGG |
| | | CGGCGGCUCCCCCAAGAAGAAGCGGAAGGUGUCCGAGUCCGCCAC |
| | | CCCCGAGUCCGUGUCCGGCUGGCGGCUGUUCAAGAAGAUCUCCUG |
| | | A |
| 6 | Amino acid | MEASPASGPRHLMDPHIFTSNFNNGIGRHKTYLCYEVERLDNGTSVKM |
| | sequence for | DQHRGFLHNQAKNLLCGFYGRHAELRFLDLVPSLQLDPAQIYRVTWFI |
| | BC22n with Hibit | SWSPCFSWGCAGEVRAFLQENTHVRLRIFAARIYDYDPLYKEALQMLR |
| | tag | DAGAQVSIMTYDEFKHCWDTFVDHQGCPFQPWDGLDEHSQALSGRL |
| | | RAILQNQGNSGSETPGTSESATPESDKKYSIGLAIGTNSVGWAVITDEY |
| | | KVPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGNIV |
| | | DEVAYHEKYPTIYHLRKKLVDSTDKADLRLIYLALAHMIKFRGHFLIE |
| | | GDLNPDNSDVDKLFIQLVQTYNQLFEENPINASGVDAKAILSARLSKSR |
| | | RLENLIAQLPGEKKNGLFGNLIALSLGLTPNFKSNFDLAEDAKLQLSKD |
| | | TYDDDLDNLLAQIGDQYADLFLAAKNLSDAILLSDILRVNTEITKAPLS |
| | | ASMIKRYDEHHQDLTLLKALVRQQLPEKYKEIFFDQSKNGYAGYIDGG |
| | | ASQEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAWM |
| | | TRKSEETITPWNFEEVVDKGASAQSFIERMTNFDKNLPNEKVLPKHSLL |
| | | YEYFTVYNELTKVKYVTEGMRKPAFLSGEQKKAIVDLLFKTNRKVTV |
| | | KQLKEDYFKKIECFDSVEISGVEDRFNASLGTYHDLLKIIKDKDFLDNE |
| | | ENEDILEDIVLTLTLFEDREMIEERLKTYAHLFDDKVMKQLKRRRYTG |
| | | WGRLSRKLINGIRDKQSGKTILDFLKSDGFANRNFMQLIHDDSLTFKED |
| | | IQKAQVSGQGDSLHEHIANLAGSPAIKKGILQTVKVVDELVKVMGRHK |
| | | PENIVIEMARENQTTQKGQKNSRERMKRIEEGIKELGSQILKEHPVENT |
| | | QLQNEKLYLYYLQNGRDMYVDQELDINRLSDYDVDHIVPQSFLKDDSI |
| | | DNKVLTRSDKNRGKSDNVPSEEVVKKMKNYWRQLLNAKLITQRKFD |
| | | NLTKAERGGLSELDKAGFIKRQLVETRQITKHVAQILDSRMNTKYDEN |
| | | DKLIREVKVITLKSKLVSDFRKDFQFYKVREINNYHHAHDAYLNAVVG |
| | | TALIKKYPKLESEFVYGDYKVYDVRKMIAKSEQEIGKATAKYFFYSNI |
| | | MNFFKTEITLANGEIRKRPLIETNGETGEIVWDKGRDFATVRKVLSMPQ |
| | | VNIVKKTEVQTGGFSKESILPKRNSDKLIARKKDWDPKKYGGFDSPTV |
| | | AYSVLVVAKVEKGKSKKLKSVKELLGITIMERSSFEKNPIDFLEAKGYK |
| | | EVKKDLIIKLPKYSLFELENGRKRMLASAGELQKGNELALPSKYVNFL |
| | | YLASHYEKLKGSPEDNEQKQLFVEQHKHYLDEIIEQISEFSKRVILADA |
| | | NLDKVLSAYNKHRDKPIREQAENIIHLFTLTNLGAPAAFKYFDTTIDRK |
| | | RYTSTKEVLDATLIHQSITGLYETRIDLSQLGGDGGGSPKKKRKVSESA |
| | | TPESVSGWRLFKKIS |
| 7 | mRNA encoding | GGGAGACCCAAGCUGGCUAGCGUUUAAACUUAAGCUUUCCCGCA |
| | BE3 | GUCGGCGUCCAGCGGCUCUGCUUGUUCGUGUGUGUGUCGUUGCA |
| | | GGCCUUAUUCGGAUCCGCCACCAUGAGCAGCGAAACAGGACCGGU |
| | | CGCAGUCGACCCGACACUGAGAAGAAGAAUCGAACCGCACGAAUU |
| | | CGAAGUCUUCUUCGACCCGAGAGAACUGAGAAAGGAAACAUGCC |
| | | UGCUGUACGAAAUCAACUGGGGAGGAAGACACAGCAUCUGGAGA |
| | | CACACAAGCCAGAACACAAACAAGCACGUCGAAGUCAACUUCAUC |
| | | GAAAAGUUCACAACAGAAAGAUACUUCUGCCCGAACACAAGAUG |
| | | CAGCAUCACAUGGUUCCUGAGCUGGAGCCCGUGCGGAGAAUGCA |
| | | GCAGAGCAAUCACAGAAUUCCUGAGCAGAUACCCGCACGUCACAC |
| | | UGUUCAUCUACAUCGCAAGACUGUACCACCACGCAGACCCGAGAA |
| | | ACAGACAGGGACUGAGAGACCUGAUCAGCAGCGGAGUCACAAUC |
| | | CAGAUCAUGACAGAACAGGAAAGCGGAUACUGCUGGAGAAACUU |
| | | CGUCAACUACAGCCCGAGCAACGAAGCACACUGGCCGAGAUACCC |
| | | GCACCUGUGGGUCAGACUGUACGUCCUGGAACUGUACUGCAUCA |
| | | UCCUGGGACUGCCGCCGUGCCUGAACAUCCUGAGAAGAAAGCAGC |
| | | CGCAGCUGACAUUCUUCACAAUCGCACUGCAGAGCUGCCACUACC |
| | | AGAGACUGCCGCCGCACAUCCUGUGGGCAACAGGACUGAAGAGCG |
| | | GAAGCGAAACACCGGGAACAAGCGAAAGCGCAACACCGGAAAGC |
| | | GACAAGAAGUACAGCAUCGGACUGGCCAUCGGAACAAACAGCGU |
| | | CGGAUGGGCAGUCAUCACAGACGAAUACAAGGUCCCGAGCAAGA |
| | | AGUUCAAGGUCCUGGGAAACACAGACAGACACAGCAUCAAGAAG |
| | | AACCUGAUCGGAGCACUGCUGUUCGACAGCGGAGAAACAGCAGA |
| | | AGCAACAAGACUGAAGAGAACAGCAAGAAGAAGAUACACAAGAA |
| | | GAAAGAACAGAAUCUGCUACCUGCAGGAAAUCUUCAGCAACGAA |
| | | AUGGCAAAGGUCGACGACAGCUUCUUCCACAGACUGGAAGAAAG |
| | | CUUCCUGGUCGAAGAAGACAAGAAGCACGAAAGACACCCGAUCU |
| | | UCGGAAACAUCGUCGACGAAGUCGCAUACCACGAAAAGUACCCGA |
| | | CAAUCUACCACCUGAGAAAGAAGCUGGUCGACAGCACAGACAAG |
| | | GCAGACCUGAGACUGAUCUACCUGGCACUGGCACACAUGAUCAAG |
| | | UUCAGAGGACACUUCCUGAUCGAAGGAGACCUGAACCCGGACAAC |
| | | AGCGACGUCGACAAGCUGUUCAUCCAGCUGGUCCAGACAUACAAC |
| | | CAGCUGUUCGAAGAAAACCCGAUCAACGCAAGCGGAGUCGACGCA |
| | | AAGGCAAUCCUGAGCGCAAGACUGAGCAAGAGCAGAAGACUGGA |
| | | AAACCUGAUCGCACAGCUGCCGGGAGAAAAGAAGAACGGACUGU |
| | | UCGGAAACCUGAUCGCACUGAGCCUGGGACUGACACCGAACUUCA |
| | | AGAGCAACUUCGACCUGGCAGAAGACGCAAAGCUGCAGCUGAGC |
| | | AAGGACACAUACGACGACGACCUGGACAACCUGCUGGCACAGAUC |
| | | GGAGACCAGUACGCAGACCUGUUCCUGGCAGCAAAGAACCUGAGC |
| | | GACGCAAUCCUGCUGAGCGACAUCCUGAGAGUCAACACAGAAAUC |
| | | ACAAAGGCACCGCUGAGCGCAAGCAUGAUCAAGAGAUACGACGA |
| | | ACACCACCAGGACCUGACACUGCUGAAGGCACUGGUCAGACAGCA |
| | | GCUGCCGGAAAAGUACAAGGAAAUCUUCUUCGACCAGAGCAAGA |
| | | ACGGAUACGCAGGAUACAUCGACGGAGGAGCAAGCCAGGAAGAA |
| | | UUCUACAAGUUCAUCAAGCCGAUCCUGGAAAAGAUGGACGGAAC |
| | | AGAAGAACUGCUGGUCAAGCUGAACAGAGAAGACCUGCUGAGAA |
| | | AGCAGAGAACAUUCGACAACGGAAGCAUCCCGCACCAGAUCCACC |
| | | UGGGAGAACUGCACGCAAUCCUGAGAAGACAGGAAGACUUCUAC |
| | | CCGUUCCUGAAGGACAACAGAGAAAAGAUCGAAAAGAUCCUGAC |
| | | AUUCAGAAUCCCGUACUACGUCGGACCGCUGGCAAGAGGAAACA |
| | | GCAGAUUCGCAUGGAUGACAAGAAAGAGCGAAGAAACAAUCACA |
| | | CCGUGGAACUUCGAAGAAGUCGUCGACAAGGGAGCAAGCGCACA |
| | | GAGCUUCAUCGAAAGAAUGACAAACUUCGACAAGAACCUGCCGA |
| | | ACGAAAAGGUCCUGCCGAAGCACAGCCUGCUGUACGAAUACUUCA |
| | | CAGUCUACAACGAACUGACAAAGGUCAAGUACGUCACAGAAGGA |
| | | AUGAGAAAGCCGGCAUUCCUGAGCGGAGAACAGAAGAAGGCAAU |
| | | CGUCGACCUGCUGUUCAAGACAAACAGAAAGGUCACAGUCAAGC |
| | | AGCUGAAGGAAGACUACUUCAAGAAGAUCGAAUGCUUCGACAGC |
| | | GUCGAAAUCAGCGGAGUCGAAGACAGAUUCAACGCAAGCCUGGG |
| | | AACAUACCACGACCUGCUGAAGAUCAUCAAGGACAAGGACUUCCU |
| | | GGACAACGAAGAAAACGAAGACAUCCUGGAAGACAUCGUCCUGA |
| | | CACUGACACUGUUCGAAGACAGAGAAAUGAUCGAAGAAAGACUG |
| | | AAGACAUACGCACACCUGUUCGACGACAAGGUCAUGAAGCAGCU |
| | | GAAGAGAAGAAGAUACACAGGAUGGGGAAGACUGAGCAGAAAGC |
| | | UGAUCAACGGAAUCAGAGACAAGCAGAGCGGAAAGACAAUCCUG |
| | | GACUUCCUGAAGAGCGACGGAUUCGCAAACAGAAACUUCAUGCA |
| | | GCUGAUCCACGACGACAGCCUGACAUUCAAGGAAGACAUCCAGAA |
| | | GGCACAGGUCAGCGGACAGGGAGACAGCCUGCACGAACACAUCGC |
| | | AAACCUGGCAGGAAGCCCGGCAAUCAAGAAGGGAAUCCUGCAGA |
| | | CAGUCAAGGUCGUCGACGAACUGGUCAAGGUCAUGGGAAGACAC |
| | | AAGCCGGAAAACAUCGUCAUCGAAAUGGCAAGAGAAAACCAGAC |
| | | AACACAGAAGGGACAGAAGAACAGCAGAGAAAGAAUGAAGAGAA |
| | | UCGAAGAAGGAAUCAAGGAACUGGGAAGCCAGAUCCUGAAGGAA |
| | | CACCCGGUCGAAAACACACAGCUGCAGAACGAAAAGCUGUACCUG |
| | | UACUACCUGCAGAACGGAAGAGACAUGUACGUCGACCAGGAACU |
| | | GGACAUCAACAGACUGAGCGACUACGACGUCGACCACAUCGUCCC |
| | | GCAGAGCUUCCUGAAGGACGACAGCAUCGACAACAAGGUCCUGAC |
| | | AAGAAGCGACAAGAACAGAGGAAAGAGCGACAACGUCCCGAGCG |
| | | AAGAAGUCGUCAAGAAGAUGAAGAACUACUGGAGACAGCUGCUG |
| | | AACGCAAAGCUGAUCACACAGAGAAAGUUCGACAACCUGACAAA |
| | | GGCAGAGAGAGGAGGACUGAGCGAACUGGACAAGGCAGGAUUCA |
| | | UCAAGAGACAGCUGGUCGAAACAAGACAGAUCACAAAGCACGUC |
| | | GCACAGAUCCUGGACAGCAGAAUGAACACAAAGUACGACGAAAA |
| | | CGACAAGCUGAUCAGAGAAGUCAAGGUCAUCACACUGAAGAGCA |
| | | AGCUGGUCAGCGACUUCAGAAAGGACUUCCAGUUCUACAAGGUC |
| | | AGAGAAAUCAACAACUACCACCACGCACACGACGCAUACCUGAAC |
| | | GCAGUCGUCGGAACAGCACUGAUCAAGAAGUACCCGAAGCUGGA |
| | | AAGCGAAUUCGUCUACGGAGACUACAAGGUCUACGACGUCAGAA |
| | | AGAUGAUCGCAAAGAGCGAACAGGAAAUCGGAAAGGCAACAGCA |
| | | AAGUACUUCUUCUACAGCAACAUCAUGAACUUCUUCAAGACAGA |
| | | AAUCACACUGGCAAACGGAGAAAUCAGAAAGAGACCGCUGAUCG |
| | | AAACAAACGGAGAAACAGGAGAAAUCGUCUGGGACAAGGGAAGA |
| | | GACUUCGCAACAGUCAGAAAGGUCCUGAGCAUGCCGCAGGUCAAC |
| | | AUCGUCAAGAAGACAGAAGUCCAGACAGGAGGAUUCAGCAAGGA |
| | | AAGCAUCCUGCCGAAGAGAAACAGCGACAAGCUGAUCGCAAGAA |
| | | AGAAGGACUGGGACCCGAAGAAGUACGGAGGAUUCGACAGCCCG |
| | | ACAGUCGCAUACAGCGUCCUGGUCGUCGCAAAGGUCGAAAAGGG |
| | | AAAGAGCAAGAAGCUGAAGAGCGUCAAGGAACUGCUGGGAAUCA |
| | | CAAUCAUGGAAAGAAGCAGCUUCGAAAAGAACCCGAUCGACUUC |
| | | CUGGAAGCAAAGGGAUACAAGGAAGUCAAGAAGGACCUGAUCAU |
| | | CAAGCUGCCGAAGUACAGCCUGUUCGAACUGGAAAACGGAAGAA |
| | | AGAGAAUGCUGGCAAGCGCAGGAGAACUGCAGAAGGGAAACGAA |
| | | CUGGCACUGCCGAGCAAGUACGUCAACUUCCUGUACCUGGCAAGC |
| | | CACUACGAAAAGCUGAAGGGAAGCCCGGAAGACAACGAACAGAA |
| | | GCAGCUGUUCGUCGAACAGCACAAGCACUACCUGGACGAAAUCAU |
| | | CGAACAGAUCAGCGAAUUCAGCAAGAGAGUCAUCCUGGCAGACG |
| | | CAAACCUGGACAAGGUCCUGAGCGCAUACAACAAGCACAGAGACA |
| | | AGCCGAUCAGAGAACAGGCAGAAAACAUCAUCCACCUGUUCACAC |
| | | UGACAAACCUGGGAGCACCGGCAGCAUUCAAGUACUUCGACACAA |
| | | CAAUCGACAGAAAGAGAUACACAAGCACAAAGGAAGUCCUGGAC |
| | | GCAACACUGAUCCACCAGAGCAUCACAGGACUGUACGAAACAAGA |
| | | AUCGAUCUGAGCCAGCUGGGAGGAGACAGCGGAGGAAGCACAAA |
| | | CCUGAGCGACAUCAUCGAAAAGGAAACAGGAAAGCAGCUGGUCA |
| | | UCCAGGAAAGCAUCCUGAUGCUGCCGGAAGAAGUCGAAGAAGUC |
| | | AUCGGAAACAAGCCGGAAAGCGACAUCCUGGUCCACACAGCAUAC |
| | | GACGAAAGCACAGACGAAAACGUCAUGCUGCUGACAAGCGACGC |
| | | ACCGGAAUACAAGCCGUGGGCACUGGUCAUCCAGGACAGCAACGG |
| | | AGAAAACAAGAUCAAGAUGCUGAGCGGAGGAAGCCCGAAGAAGA |
| | | AGAGAAAGGUCUAAUAGUCUAGACAUCACAUUUAAAAGCAUCUC |
| | | AGCCUACCAUGAGAAUAAGAGAAAGAAAAUGAAGAUCAAUAGCU |
| | | UAUUCAUCUCUUUUUCUUUUUCGUUGGUGUAAAGCCAACACCCU |
| | | GUCUAAAAAACAUAAAUUUCUUUAAUCAUUUUGCCUCUUUUCUC |
| | | UGUGCUUCAAUUAAUAAAAAAUGGAAAGAACCUCGAGAAAAAAA |
| | | AAAAAAAAAAAAAAAAAAAAAAAGCGAAAAAAAAAAAAAAAAAA |
| | | AAAAAAAAAAAACCGAAAAAAAAAAAAAAAAAAAAAAAAAAAAA |
| | | AAAAAAAAAAU |
| 8 | Open reading | AUGAGCAGCGAAACAGGACCGGUCGCAGUCGACCCGACACUGAGA |
| | frame for BE3 | AGAAGAAUCGAACCGCACGAAUUCGAAGUCUUCUUCGACCCGAG |
| | | AGAACUGAGAAAGGAAACAUGCCUGCUGUACGAAAUCAACUGGG |
| | | GAGGAAGACACAGCAUCUGGAGACACACAAGCCAGAACACAAAC |
| | | AAGCACGUCGAAGUCAACUUCAUCGAAAAGUUCACAACAGAAAG |
| | | AUACUUCUGCCCGAACACAAGAUGCAGCAUCACAUGGUUCCUGAG |
| | | CUGGAGCCCGUGCGGAGAAUGCAGCAGAGCAAUCACAGAAUUCC |
| | | UGAGCAGAUACCCGCACGUCACACUGUUCAUCUACAUCGCAAGAC |
| | | UGUACCACCACGCAGACCCGAGAAACAGACAGGGACUGAGAGACC |
| | | UGAUCAGCAGCGGAGUCACAAUCCAGAUCAUGACAGAACAGGAA |
| | | AGCGGAUACUGCUGGAGAAACUUCGUCAACUACAGCCCGAGCAAC |
| | | GAAGCACACUGGCCGAGAUACCCGCACCUGUGGGUCAGACUGUAC |
| | | GUCCUGGAACUGUACUGCAUCAUCCUGGGACUGCCGCCGUGCCUG |
| | | AACAUCCUGAGAAGAAAGCAGCCGCAGCUGACAUUCUUCACAAUC |
| | | GCACUGCAGAGCUGCCACUACCAGAGACUGCCGCCGCACAUCCUG |
| | | UGGGCAACAGGACUGAAGAGCGGAAGCGAAACACCGGGAACAAG |
| | | CGAAAGCGCAACACCGGAAAGCGACAAGAAGUACAGCAUCGGAC |
| | | UGGCCAUCGGAACAAACAGCGUCGGAUGGGCAGUCAUCACAGAC |
| | | GAAUACAAGGUCCCGAGCAAGAAGUUCAAGGUCCUGGGAAACAC |
| | | AGACAGACACAGCAUCAAGAAGAACCUGAUCGGAGCACUGCUGU |
| | | UCGACAGCGGAGAAACAGCAGAAGCAACAAGACUGAAGAGAACA |
| | | GCAAGAAGAAGAUACACAAGAAGAAAGAACAGAAUCUGCUACCU |
| | | GCAGGAAAUCUUCAGCAACGAAAUGGCAAAGGUCGACGACAGCU |
| | | UCUUCCACAGACUGGAAGAAAGCUUCCUGGUCGAAGAAGACAAG |
| | | AAGCACGAAAGACACCCGAUCUUCGGAAACAUCGUCGACGAAGUC |
| | | GCAUACCACGAAAAGUACCCGACAAUCUACCACCUGAGAAAGAAG |
| | | CUGGUCGACAGCACAGACAAGGCAGACCUGAGACUGAUCUACCUG |
| | | GCACUGGCACACAUGAUCAAGUUCAGAGGACACUUCCUGAUCGA |
| | | AGGAGACCUGAACCCGGACAACAGCGACGUCGACAAGCUGUUCAU |
| | | CCAGCUGGUCCAGACAUACAACCAGCUGUUCGAAGAAAACCCGAU |
| | | CAACGCAAGCGGAGUCGACGCAAAGGCAAUCCUGAGCGCAAGACU |
| | | GAGCAAGAGCAGAAGACUGGAAAACCUGAUCGCACAGCUGCCGG |
| | | GAGAAAAGAAGAACGGACUGUUCGGAAACCUGAUCGCACUGAGC |
| | | CUGGGACUGACACCGAACUUCAAGAGCAACUUCGACCUGGCAGAA |
| | | GACGCAAAGCUGCAGCUGAGCAAGGACACAUACGACGACGACCUG |
| | | GACAACCUGCUGGCACAGAUCGGAGACCAGUACGCAGACCUGUUC |
| | | CUGGCAGCAAAGAACCUGAGCGACGCAAUCCUGCUGAGCGACAUC |
| | | CUGAGAGUCAACACAGAAAUCACAAAGGCACCGCUGAGCGCAAGC |
| | | AUGAUCAAGAGAUACGACGAACACCACCAGGACCUGACACUGCUG |
| | | AAGGCACUGGUCAGACAGCAGCUGCCGGAAAAGUACAAGGAAAU |
| | | CUUCUUCGACCAGAGCAAGAACGGAUACGCAGGAUACAUCGACG |
| | | GAGGAGCAAGCCAGGAAGAAUUCUACAAGUUCAUCAAGCCGAUC |
| | | CUGGAAAAGAUGGACGGAACAGAAGAACUGCUGGUCAAGCUGAA |
| | | CAGAGAAGACCUGCUGAGAAAGCAGAGAACAUUCGACAACGGAA |
| | | GCAUCCCGCACCAGAUCCACCUGGGAGAACUGCACGCAAUCCUGA |
| | | GAAGACAGGAAGACUUCUACCCGUUCCUGAAGGACAACAGAGAA |
| | | AAGAUCGAAAAGAUCCUGACAUUCAGAAUCCCGUACUACGUCGG |
| | | ACCGCUGGCAAGAGGAAACAGCAGAUUCGCAUGGAUGACAAGAA |
| | | AGAGCGAAGAAACAAUCACACCGUGGAACUUCGAAGAAGUCGUC |
| | | GACAAGGGAGCAAGCGCACAGAGCUUCAUCGAAAGAAUGACAAA |
| | | CUUCGACAAGAACCUGCCGAACGAAAAGGUCCUGCCGAAGCACAG |
| | | CCUGCUGUACGAAUACUUCACAGUCUACAACGAACUGACAAAGG |
| | | UCAAGUACGUCACAGAAGGAAUGAGAAAGCCGGCAUUCCUGAGC |
| | | GGAGAACAGAAGAAGGCAAUCGUCGACCUGCUGUUCAAGACAAA |
| | | CAGAAAGGUCACAGUCAAGCAGCUGAAGGAAGACUACUUCAAGA |
| | | AGAUCGAAUGCUUCGACAGCGUCGAAAUCAGCGGAGUCGAAGAC |
| | | AGAUUCAACGCAAGCCUGGGAACAUACCACGACCUGCUGAAGAUC |
| | | AUCAAGGACAAGGACUUCCUGGACAACGAAGAAAACGAAGACAU |
| | | CCUGGAAGACAUCGUCCUGACACUGACACUGUUCGAAGACAGAG |
| | | AAAUGAUCGAAGAAAGACUGAAGACAUACGCACACCUGUUCGAC |
| | | GACAAGGUCAUGAAGCAGCUGAAGAGAAGAAGAUACACAGGAUG |
| | | GGGAAGACUGAGCAGAAAGCUGAUCAACGGAAUCAGAGACAAGC |
| | | AGAGCGGAAAGACAAUCCUGGACUUCCUGAAGAGCGACGGAUUC |
| | | GCAAACAGAAACUUCAUGCAGCUGAUCCACGACGACAGCCUGACA |
| | | UUCAAGGAAGACAUCCAGAAGGCACAGGUCAGCGGACAGGGAGA |
| | | CAGCCUGCACGAACACAUCGCAAACCUGGCAGGAAGCCCGGCAAU |
| | | CAAGAAGGGAAUCCUGCAGACAGUCAAGGUCGUCGACGAACUGG |
| | | UCAAGGUCAUGGGAAGACACAAGCCGGAAAACAUCGUCAUCGAA |
| | | AUGGCAAGAGAAAACCAGACAACACAGAAGGGACAGAAGAACAG |
| | | CAGAGAAAGAAUGAAGAGAAUCGAAGAAGGAAUCAAGGAACUGG |
| | | GAAGCCAGAUCCUGAAGGAACACCCGGUCGAAAACACACAGCUGC |
| | | AGAACGAAAAGCUGUACCUGUACUACCUGCAGAACGGAAGAGAC |
| | | AUGUACGUCGACCAGGAACUGGACAUCAACAGACUGAGCGACUA |
| | | CGACGUCGACCACAUCGUCCCGCAGAGCUUCCUGAAGGACGACAG |
| | | CAUCGACAACAAGGUCCUGACAAGAAGCGACAAGAACAGAGGAA |
| | | AGAGCGACAACGUCCCGAGCGAAGAAGUCGUCAAGAAGAUGAAG |
| | | AACUACUGGAGACAGCUGCUGAACGCAAAGCUGAUCACACAGAG |
| | | AAAGUUCGACAACCUGACAAAGGCAGAGAGAGGAGGACUGAGCG |
| | | AACUGGACAAGGCAGGAUUCAUCAAGAGACAGCUGGUCGAAACA |
| | | AGACAGAUCACAAAGCACGUCGCACAGAUCCUGGACAGCAGAAU |
| | | GAACACAAAGUACGACGAAAACGACAAGCUGAUCAGAGAAGUCA |
| | | AGGUCAUCACACUGAAGAGCAAGCUGGUCAGCGACUUCAGAAAG |
| | | GACUUCCAGUUCUACAAGGUCAGAGAAAUCAACAACUACCACCAC |
| | | GCACACGACGCAUACCUGAACGCAGUCGUCGGAACAGCACUGAUC |
| | | AAGAAGUACCCGAAGCUGGAAAGCGAAUUCGUCUACGGAGACUA |
| | | CAAGGUCUACGACGUCAGAAAGAUGAUCGCAAAGAGCGAACAGG |
| | | AAAUCGGAAAGGCAACAGCAAAGUACUUCUUCUACAGCAACAUC |
| | | AUGAACUUCUUCAAGACAGAAAUCACACUGGCAAACGGAGAAAU |
| | | CAGAAAGAGACCGCUGAUCGAAACAAACGGAGAAACAGGAGAAA |
| | | UCGUCUGGGACAAGGGAAGAGACUUCGCAACAGUCAGAAAGGUC |
| | | CUGAGCAUGCCGCAGGUCAACAUCGUCAAGAAGACAGAAGUCCA |
| | | GACAGGAGGAUUCAGCAAGGAAAGCAUCCUGCCGAAGAGAAACA |
| | | GCGACAAGCUGAUCGCAAGAAAGAAGGACUGGGACCCGAAGAAG |
| | | UACGGAGGAUUCGACAGCCCGACAGUCGCAUACAGCGUCCUGGUC |
| | | GUCGCAAAGGUCGAAAAGGGAAAGAGCAAGAAGCUGAAGAGCGU |
| | | CAAGGAACUGCUGGGAAUCACAAUCAUGGAAAGAAGCAGCUUCG |
| | | AAAAGAACCCGAUCGACUUCCUGGAAGCAAAGGGAUACAAGGAA |
| | | GUCAAGAAGGACCUGAUCAUCAAGCUGCCGAAGUACAGCCUGUU |
| | | CGAACUGGAAAACGGAAGAAAGAGAAUGCUGGCAAGCGCAGGAG |
| | | AACUGCAGAAGGGAAACGAACUGGCACUGCCGAGCAAGUACGUC |
| | | AACUUCCUGUACCUGGCAAGCCACUACGAAAAGCUGAAGGGAAG |
| | | CCCGGAAGACAACGAACAGAAGCAGCUGUUCGUCGAACAGCACAA |
| | | GCACUACCUGGACGAAAUCAUCGAACAGAUCAGCGAAUUCAGCA |
| | | AGAGAGUCAUCCUGGCAGACGCAAACCUGGACAAGGUCCUGAGC |
| | | GCAUACAACAAGCACAGAGACAAGCCGAUCAGAGAACAGGCAGA |
| | | AAACAUCAUCCACCUGUUCACACUGACAAACCUGGGAGCACCGGC |
| | | AGCAUUCAAGUACUUCGACACAACAAUCGACAGAAAGAGAUACA |
| | | CAAGCACAAAGGAAGUCCUGGACGCAACACUGAUCCACCAGAGCA |
| | | UCACAGGACUGUACGAAACAAGAAUCGAUCUGAGCCAGCUGGGA |
| | | GGAGACAGCGGAGGAAGCACAAACCUGAGCGACAUCAUCGAAAA |
| | | GGAAACAGGAAAGCAGCUGGUCAUCCAGGAAAGCAUCCUGAUGC |
| | | UGCCGGAAGAAGUCGAAGAAGUCAUCGGAAACAAGCCGGAAAGC |
| | | GACAUCCUGGUCCACACAGCAUACGACGAAAGCACAGACGAAAAC |
| | | GUCAUGCUGCUGACAAGCGACGCACCGGAAUACAAGCCGUGGGCA |
| | | CUGGUCAUCCAGGACAGCAACGGAGAAAACAAGAUCAAGAUGCU |
| | | GAGCGGAGGAAGCCCGAAGAAGAAGAGAAAGGUC |
| 9 | Amino acid | MSSETGPVAVDPTLRRRIEPHEFEVFFDPRELRKETCLLYEINWGGRHSI |
| | sequence for BE3 | WRHTSQNTNKHVEVNFIEKFTTERYFCPNTRCSITWFLSWSPCGECSRA |
| | | ITEFLSRYPHVTLFIYIARLYHHADPRNRQGLRDLISSGVTIQIMTEQESG |
| | | YCWRNFVNYSPSNEAHWPRYPHLWVRLYVLELYCIILGLPPCLNILRR |
| | | KQPQLTFFTIALQSCHYQRLPPHILWATGLKSGSETPGTSESATPESDKK |
| | | YSIGLAIGTNSVGWAVITDEYKVPSKKFKVLGNTDRHSIKKNLIGALLF |
| | | DSGETAEATRLKRTARRRYTRRKNRICYLQEIFSNEMAKVDDSFFHRL |
| | | EESFLVEEDKKHERHPIFGNIVDEVAYHEKYPTIYHLRKKLVDSTDKAD |
| | | LRLIYLALAHMIKFRGHFLIEGDLNPDNSDVDKLFIQLVQTYNQLFEEN |
| | | PINASGVDAKAILSARLSKSRRLENLIAQLPGEKKNGLFGNLIALSLGLT |
| | | PNFKSNFDLAEDAKLQLSKDTYDDDLDNLLAQIGDQYADLFLAAKNL |
| | | SDAILLSDILRVNTEITKAPLSASMIKRYDEHHQDLTLLKALVRQQLPE |
| | | KYKEIFFDQSKNGYAGYIDGGASQEEFYKFIKPILEKMDGTEELLVKLN |
| | | REDLLRKQRTFDNGSIPHQIHLGELHAILRRQEDFYPFLKDNREKIEKIL |
| | | TFRIPYYVGPLARGNSRFAWMTRKSEETITPWNFEEVVDKGASAQSFIE |
| | | RMTNFDKNLPNEKVLPKHSLLYEYFTVYNELTKVKYVTEGMRKPAFL |
| | | SGEQKKAIVDLLFKTNRKVTVKQLKEDYFKKIECFDSVEISGVEDRFNA |
| | | SLGTYHDLLKIIKDKDFLDNEENEDILEDIVLTLTLFEDREMIEERLKTY |
| | | AHLFDDKVMKQLKRRRYTGWGRLSRKLINGIRDKQSGKTILDFLKSD |
| | | GFANRNFMQLIHDDSLTFKEDIQKAQVSGQGDSLHEHIANLAGSPAIK |
| | | KGILQTVKVVDELVKVMGRHKPENIVIEMARENQTTQKGQKNSRERM |
| | | KRIEEGIKELGSQILKEHPVENTQLQNEKLYLYYLQNGRDMYVDQELD |
| | | INRLSDYDVDHIVPQSFLKDDSIDNKVLTRSDKNRGKSDNVPSEEVVK |
| | | KMKNYWRQLLNAKLITQRKFDNLTKAERGGLSELDKAGFIKRQLVET |
| | | RQITKHVAQILDSRMNTKYDENDKLIREVKVITLKSKLVSDFRKDFQFY |
| | | KVREINNYHHAHDAYLNAVVGTALIKKYPKLESEFVYGDYKVYDVRK |
| | | MIAKSEQEIGKATAKYFFYSNIMNFFKTEITLANGEIRKRPLIETNGETG |
| | | EIVWDKGRDFATVRKVLSMPQVNIVKKTEVQTGGFSKESILPKRNSDK |
| | | LIARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRMLA |
| | | SAGELQKGNELALPSKYVNFLYLASHYEKLKGSPEDNEQKQLFVEQH |
| | | KHYLDEIIEQISEFSKRVILADANLDKVLSAYNKHRDKPIREQAENIIHL |
| | | FTLTNLGAPAAFKYFDTTIDRKRYTSTKEVLDATLIHQSITGLYETRIDL |
| | | SQLGGDSGGSTNLSDIIEKETGKQLVIQESILMLPEEVEEVIGNKPESDIL |
| | | VHTAYDESTDENVMLLTSDAPEYKPWALVIQDSNGENKIKMLSGGSP |
| | | KKKRKV |
| 10 | mRNA encoding | GGGUCCCGCAGUCGGCGUCCAGCGGCUCUGCUUGUUCGUGUGUGU |
| | BE3 | GUCGUUGCAGGCCUUAUUCGGAUCCACCAUGAGCUCAGAGACUG |
| | | GCCCAGUGGCUGUGGACCCCACAUUGAGACGGCGGAUCGAGCCCC |
| | | AUGAGUUUGAGGUAUUCUUCGAUCCGAGAGAGCUCCGCAAGGAG |
| | | ACCUGCCUGCUUUACGAAAUUAAUUGGGGGGGCCGGCACUCCAU |
| | | UUGGCGACAUACAUCACAGAACACUAACAAGCACGUCGAAGUCA |
| | | ACUUCAUCGAGAAGUUCACGACAGAAAGAUAUUUCUGUCCGAAC |
| | | ACAAGGUGCAGCAUUACCUGGUUUCUCAGCUGGAGCCCAUGCGGC |
| | | GAAUGUAGUAGGGCCAUCACUGAAUUCCUGUCAAGGUAUCCCCA |
| | | CGUCACUCUGUUUAUUUACAUCGCAAGGCUGUACCACCACGCUGA |
| | | CCCCCGCAAUCGACAAGGCCUGCGGGAUUUGAUCUCUUCAGGUGU |
| | | GACUAUCCAAAUUAUGACUGAGCAGGAGUCAGGAUACUGCUGGA |
| | | GAAACUUUGUGAAUUAUAGCCCGAGUAAUGAAGCCCACUGGCCU |
| | | AGGUAUCCCCAUCUGUGGGUACGACUGUACGUUCUUGAACUGUA |
| | | CUGCAUCAUACUGGGCCUGCCUCCUUGUCUCAACAUUCUGAGAAG |
| | | GAAGCAGCCACAGCUGACAUUCUUUACCAUCGCUCUUCAGUCUUG |
| | | UCAUUACCAGCGACUGCCCCCACACAUUCUCUGGGCCACCGGGUU |
| | | GAAAAGCGGCAGCGAGACUCCGGGCACCUCAGAGUCCGCCACACC |
| | | CGAAAGUGAUAAGAAGUACUCAAUCGGGCUGGCCAUCGGAACUA |
| | | AUUCCGUGGGUUGGGCAGUGAUCACGGAUGAAUACAAAGUGCCG |
| | | UCCAAGAAGUUCAAGGUCCUGGGGAACACCGAUAGACACAGCAU |
| | | CAAGAAAAAUCUCAUCGGAGCCCUGCUGUUUGACUCCGGCGAAAC |
| | | CGCAGAAGCGACCCGGCUCAAACGUACCGCGAGGCGACGCUACAC |
| | | CCGGCGGAAGAAUCGCAUCUGCUAUCUGCAAGAGAUCUUUUCGA |
| | | ACGAAAUGGCAAAGGUCGACGACAGCUUCUUCCACCGCCUGGAAG |
| | | AAUCUUUCCUGGUGGAGGAGGACAAGAAGCAUGAACGGCAUCCU |
| | | AUCUUUGGAAACAUCGUCGACGAAGUGGCGUACCACGAAAAGUA |
| | | CCCGACCAUCUACCAUCUGCGGAAGAAGUUGGUUGACUCAACUGA |
| | | CAAGGCCGACCUCAGAUUGAUCUACUUGGCCCUCGCCCAUAUGAU |
| | | CAAAUUCCGCGGACACUUCCUGAUCGAAGGCGAUCUGAACCCUGA |
| | | UAACUCCGACGUGGAUAAGCUUUUCAUUCAACUGGUGCAGACCU |
| | | ACAACCAACUGUUCGAAGAAAACCCAAUCAAUGCUAGCGGCGUCG |
| | | AUGCCAAGGCCAUCCUGUCCGCCCGGCUGUCGAAGUCGCGGCGCC |
| | | UCGAAAACCUGAUCGCACAGCUGCCGGGAGAGAAAAAGAACGGA |
| | | CUUUUCGGCAACUUGAUCGCUCUCUCACUGGGACUCACUCCCAAU |
| | | UUCAAGUCCAAUUUUGACCUGGCCGAGGACGCGAAGCUGCAACUC |
| | | UCAAAGGACACCUACGACGACGACUUGGACAAUUUGCUGGCACA |
| | | AAUUGGCGAUCAGUACGCGGAUCUGUUCCUUGCCGCUAAGAACC |
| | | UUUCGGACGCAAUCUUGCUGUCCGAUAUCCUGCGCGUGAACACCG |
| | | AAAUAACCAAAGCGCCGCUUAGCGCCUCGAUGAUUAAGCGGUAC |
| | | GACGAGCAUCACCAGGAUCUCACGCUGCUCAAAGCGCUCGUGAGA |
| | | CAGCAACUGCCUGAAAAGUACAAGGAGAUCUUCUUCGACCAGUCC |
| | | AAGAAUGGGUACGCAGGGUACAUCGAUGGAGGCGCUAGCCAGGA |
| | | AGAGUUCUAUAAGUUCAUCAAGCCAAUCCUGGAAAAGAUGGACG |
| | | GAACCGAAGAACUGCUGGUCAAGCUGAACAGGGAGGAUCUGCUC |
| | | CGGAAACAGAGAACCUUUGACAACGGAUCCAUUCCCCACCAGAUC |
| | | CAUCUGGGUGAGCUGCACGCCAUCUUGCGGCGCCAGGAGGACUUU |
| | | UACCCAUUCCUCAAGGACAACCGGGAAAAGAUCGAGAAAAUUCU |
| | | GACGUUCCGCAUCCCGUAUUACGUGGGCCCACUGGCGCGCGGCAA |
| | | UUCGCGCUUCGCGUGGAUGACUAGAAAAUCAGAGGAAACCAUCA |
| | | CUCCUUGGAAUUUCGAGGAAGUUGUGGAUAAGGGAGCUUCGGCA |
| | | CAAAGCUUCAUCGAACGAAUGACCAACUUCGACAAGAAUCUCCCA |
| | | AACGAGAAGGUGCUUCCUAAGCACAGCCUCCUUUACGAAUACUUC |
| | | ACUGUCUACAACGAACUGACUAAAGUGAAAUACGUUACUGAAGG |
| | | AAUGAGGAAGCCGGCCUUUCUGUCCGGAGAACAGAAGAAAGCAA |
| | | UUGUCGAUCUGCUGUUCAAGACCAACCGCAAGGUGACCGUCAAGC |
| | | AGCUUAAAGAGGACUACUUCAAGAAGAUCGAGUGUUUCGACUCA |
| | | GUGGAAAUCAGCGGGGUGGAGGACAGAUUCAACGCUUCGCUGGG |
| | | AACCUAUCAUGAUCUCCUGAAGAUCAUCAAGGACAAGGACUUCC |
| | | UUGACAACGAGGAGAACGAGGACAUCCUGGAAGAUAUCGUCCUG |
| | | ACCUUGACCCUUUUCGAGGAUCGCGAGAUGAUCGAGGAGAGGCU |
| | | UAAGACCUACGCUCAUCUCUUCGACGAUAAGGUCAUGAAACAAC |
| | | UCAAGCGCCGCCGGUACACUGGUUGGGGCCGCCUCUCCCGCAAGC |
| | | UGAUCAACGGUAUUCGCGAUAAACAGAGCGGUAAAACUAUCCUG |
| | | GAUUUCCUCAAAUCGGAUGGCUUCGCUAAUCGUAACUUCAUGCA |
| | | AUUGAUCCACGACGACAGCCUGACCUUUAAGGAGGACAUCCAAA |
| | | AAGCACAAGUGUCCGGACAGGGAGACUCACUCCAUGAACACAUCG |
| | | CGAAUCUGGCCGGUUCGCCGGCGAUUAAGAAGGGAAUUCUGCAA |
| | | ACUGUGAAGGUGGUCGACGAGCUGGUGAAGGUCAUGGGACGGCA |
| | | CAAACCGGAGAAUAUCGUGAUUGAAAUGGCCCGAGAAAACCAGA |
| | | CUACCCAGAAGGGCCAGAAAAACUCCCGCGAAAGGAUGAAGCGG |
| | | AUCGAAGAAGGAAUCAAGGAGCUGGGCAGCCAGAUCCUGAAAGA |
| | | GCACCCGGUGGAAAACACGCAGCUGCAGAACGAGAAGCUCUACCU |
| | | GUACUAUUUGCAAAAUGGACGGGACAUGUACGUGGACCAAGAGC |
| | | UGGACAUCAAUCGGUUGUCUGAUUACGACGUGGACCACAUCGUU |
| | | CCACAGUCCUUUCUGAAGGAUGACUCGAUCGAUAACAAGGUGUU |
| | | GACUCGCAGCGACAAGAACAGAGGGAAGUCAGAUAAUGUGCCAU |
| | | CGGAGGAGGUCGUGAAGAAGAUGAAGAAUUACUGGCGGCAGCUC |
| | | CUGAAUGCGAAGCUGAUUACCCAGAGAAAGUUUGACAAUCUCAC |
| | | UAAAGCCGAGCGCGGCGGACUCUCAGAGCUGGAUAAGGCUGGAU |
| | | UCAUCAAACGGCAGCUGGUCGAGACUCGGCAGAUUACCAAGCACG |
| | | UGGCGCAGAUCUUGGACUCCCGCAUGAACACUAAAUACGACGAG |
| | | AACGAUAAGCUCAUCCGGGAAGUGAAGGUGAUUACCCUGAAAAG |
| | | CAAACUUGUGUCGGACUUUCGGAAGGACUUUCAGUUUUACAAAG |
| | | UGAGAGAAAUCAACAACUACCAUCACGCGCAUGACGCAUACCUCA |
| | | ACGCUGUGGUCGGUACCGCCCUGAUCAAAAAGUACCCUAAACUUG |
| | | AAUCGGAGUUUGUGUACGGAGACUACAAGGUCUACGACGUGAGG |
| | | AAGAUGAUAGCCAAGUCCGAACAGGAAAUCGGGAAAGCAACUGC |
| | | GAAAUACUUCUUUUACUCAAACAUCAUGAACUUUUUCAAGACUG |
| | | AAAUUACGCUGGCCAAUGGAGAAAUCAGGAAGAGGCCACUGAUC |
| | | GAAACUAACGGAGAAACGGGCGAAAUCGUGUGGGACAAGGGCAG |
| | | GGACUUCGCAACUGUUCGCAAAGUGCUCUCUAUGCCGCAAGUCAA |
| | | UAUUGUGAAGAAAACCGAAGUGCAAACCGGCGGAUUUUCAAAGG |
| | | AAUCGAUCCUCCCAAAGAGAAAUAGCGACAAGCUCAUUGCACGCA |
| | | AGAAAGACUGGGACCCGAAGAAGUACGGAGGAUUCGAUUCGCCG |
| | | ACUGUCGCAUACUCCGUCCUCGUGGUGGCCAAGGUGGAGAAGGG |
| | | AAAGAGCAAAAAGCUCAAAUCCGUCAAAGAGCUGCUGGGGAUUA |
| | | CCAUCAUGGAACGAUCCUCGUUCGAGAAGAACCCGAUUGAUUUCC |
| | | UCGAGGCGAAGGGUUACAAGGAGGUGAAGAAGGAUCUGAUCAUC |
| | | AAACUCCCCAAGUACUCACUGUUCGAACUGGAAAAUGGUCGGAA |
| | | GCGCAUGCUGGCUUCGGCCGGAGAACUCCAAAAAGGAAAUGAGC |
| | | UGGCCUUGCCUAGCAAGUACGUCAACUUCCUCUAUCUUGCUUCGC |
| | | ACUACGAAAAACUCAAAGGGUCACCGGAAGAUAACGAACAGAAG |
| | | CAGCUUUUCGUGGAGCAGCACAAGCAUUAUCUGGAUGAAAUCAU |
| | | CGAACAAAUCUCCGAGUUUUCAAAGCGCGUGAUCCUCGCCGACGC |
| | | CAACCUCGACAAAGUCCUGUCGGCCUACAAUAAGCAUAGAGAUA |
| | | AGCCGAUCAGAGAACAGGCCGAGAACAUUAUCCACUUGUUCACCC |
| | | UGACUAACCUGGGAGCCCCAGCCGCCUUCAAGUACUUCGAUACUA |
| | | CUAUCGAUCGCAAAAGAUACACGUCCACCAAGGAAGUUCUGGAC |
| | | GCGACCCUGAUCCACCAAAGCAUCACUGGACUCUACGAAACUAGG |
| | | AUCGAUCUGUCGCAGCUGGGUGGCGAUUCUGGUGGUUCUACUAA |
| | | UCUGUCAGAUAUUAUUGAAAAGGAGACCGGUAAGCAACUGGUUA |
| | | UCCAGGAAUCCAUCCUCAUGCUCCCAGAGGAGGUGGAAGAAGUC |
| | | AUUGGGAACAAGCCGGAAAGCGAUAUACUCGUGCACACCGCCUAC |
| | | GACGAGAGCACCGACGAGAAUGUCAUGCUUCUGACUAGCGACGCC |
| | | CCUGAAUACAAGCCUUGGGCUCUGGUCAUACAGGAUAGCAACGG |
| | | UGAGAACAAGAUUAAGAUGCUCUCUGGUGGUUCUCCCAAGAAGA |
| | | AGAGGAAAGUCUAAUAGUCUAGCCAUCACAUUUAAAAGCAUCUC |
| | | AGCCUACCAUGAGAAUAAGAGAAAGAAAAUGAAGAUCAAUAGCU |
| | | UAUUCAUCUCUUUUUCUUUUUCGUUGGUGUAAAGCCAACACCCU |
| | | GUCUAAAAAACAUAAAUUUCUUUAAUCAUUUUGCCUCUUUUCUC |
| | | UGUGCUUCAAUUAAUAAAAAAUGGAAAGAACCUCGAGAAAAAAA |
| | | AAAAAAAAAAAAAAAAAAAAAAAGCGAAAAAAAAAAAAAAAAAA |
| | | AAAAAAAAAAAACCGAAAAAAAAAAAAAAAAAAAAAAAAAAAAA |
| | | AAAAAAAAAAU |
| 11 | Open reading | AUGAGCUCAGAGACUGGCCCAGUGGCUGUGGACCCCACAUUGAG |
| | frame for BE3 | ACGGCGGAUCGAGCCCCAUGAGUUUGAGGUAUUCUUCGAUCCGA |
| | | GAGAGCUCCGCAAGGAGACCUGCCUGCUUUACGAAAUUAAUUGG |
| | | GGGGGCCGGCACUCCAUUUGGCGACAUACAUCACAGAACACUAAC |
| | | AAGCACGUCGAAGUCAACUUCAUCGAGAAGUUCACGACAGAAAG |
| | | AUAUUUCUGUCCGAACACAAGGUGCAGCAUUACCUGGUUUCUCA |
| | | GCUGGAGCCCAUGCGGCGAAUGUAGUAGGGCCAUCACUGAAUUC |
| | | CUGUCAAGGUAUCCCCACGUCACUCUGUUUAUUUACAUCGCAAGG |
| | | CUGUACCACCACGCUGACCCCCGCAAUCGACAAGGCCUGCGGGAU |
| | | UUGAUCUCUUCAGGUGUGACUAUCCAAAUUAUGACUGAGCAGGA |
| | | GUCAGGAUACUGCUGGAGAAACUUUGUGAAUUAUAGCCCGAGUA |
| | | AUGAAGCCCACUGGCCUAGGUAUCCCCAUCUGUGGGUACGACUGU |
| | | ACGUUCUUGAACUGUACUGCAUCAUACUGGGCCUGCCUCCUUGUC |
| | | UCAACAUUCUGAGAAGGAAGCAGCCACAGCUGACAUUCUUUACC |
| | | AUCGCUCUUCAGUCUUGUCAUUACCAGCGACUGCCCCCACACAUU |
| | | CUCUGGGCCACCGGGUUGAAAAGCGGCAGCGAGACUCCGGGCACC |
| | | UCAGAGUCCGCCACACCCGAAAGUGAUAAGAAGUACUCAAUCGG |
| | | GCUGGCCAUCGGAACUAAUUCCGUGGGUUGGGCAGUGAUCACGG |
| | | AUGAAUACAAAGUGCCGUCCAAGAAGUUCAAGGUCCUGGGGAAC |
| | | ACCGAUAGACACAGCAUCAAGAAAAAUCUCAUCGGAGCCCUGCUG |
| | | UUUGACUCCGGCGAAACCGCAGAAGCGACCCGGCUCAAACGUACC |
| | | GCGAGGCGACGCUACACCCGGCGGAAGAAUCGCAUCUGCUAUCUG |
| | | CAAGAGAUCUUUUCGAACGAAAUGGCAAAGGUCGACGACAGCUU |
| | | CUUCCACCGCCUGGAAGAAUCUUUCCUGGUGGAGGAGGACAAGA |
| | | AGCAUGAACGGCAUCCUAUCUUUGGAAACAUCGUCGACGAAGUG |
| | | GCGUACCACGAAAAGUACCCGACCAUCUACCAUCUGCGGAAGAAG |
| | | UUGGUUGACUCAACUGACAAGGCCGACCUCAGAUUGAUCUACUU |
| | | GGCCCUCGCCCAUAUGAUCAAAUUCCGCGGACACUUCCUGAUCGA |
| | | AGGCGAUCUGAACCCUGAUAACUCCGACGUGGAUAAGCUUUUCA |
| | | UUCAACUGGUGCAGACCUACAACCAACUGUUCGAAGAAAACCCAA |
| | | UCAAUGCUAGCGGCGUCGAUGCCAAGGCCAUCCUGUCCGCCCGGC |
| | | UGUCGAAGUCGCGGCGCCUCGAAAACCUGAUCGCACAGCUGCCGG |
| | | GAGAGAAAAAGAACGGACUUUUCGGCAACUUGAUCGCUCUCUCA |
| | | CUGGGACUCACUCCCAAUUUCAAGUCCAAUUUUGACCUGGCCGAG |
| | | GACGCGAAGCUGCAACUCUCAAAGGACACCUACGACGACGACUUG |
| | | GACAAUUUGCUGGCACAAAUUGGCGAUCAGUACGCGGAUCUGUU |
| | | CCUUGCCGCUAAGAACCUUUCGGACGCAAUCUUGCUGUCCGAUAU |
| | | CCUGCGCGUGAACACCGAAAUAACCAAAGCGCCGCUUAGCGCCUC |
| | | GAUGAUUAAGCGGUACGACGAGCAUCACCAGGAUCUCACGCUGC |
| | | UCAAAGCGCUCGUGAGACAGCAACUGCCUGAAAAGUACAAGGAG |
| | | AUCUUCUUCGACCAGUCCAAGAAUGGGUACGCAGGGUACAUCGA |
| | | UGGAGGCGCUAGCCAGGAAGAGUUCUAUAAGUUCAUCAAGCCAA |
| | | UCCUGGAAAAGAUGGACGGAACCGAAGAACUGCUGGUCAAGCUG |
| | | AACAGGGAGGAUCUGCUCCGGAAACAGAGAACCUUUGACAACGG |
| | | AUCCAUUCCCCACCAGAUCCAUCUGGGUGAGCUGCACGCCAUCUU |
| | | GCGGCGCCAGGAGGACUUUUACCCAUUCCUCAAGGACAACCGGGA |
| | | AAAGAUCGAGAAAAUUCUGACGUUCCGCAUCCCGUAUUACGUGG |
| | | GCCCACUGGCGCGCGGCAAUUCGCGCUUCGCGUGGAUGACUAGAA |
| | | AAUCAGAGGAAACCAUCACUCCUUGGAAUUUCGAGGAAGUUGUG |
| | | GAUAAGGGAGCUUCGGCACAAAGCUUCAUCGAACGAAUGACCAA |
| | | CUUCGACAAGAAUCUCCCAAACGAGAAGGUGCUUCCUAAGCACAG |
| | | CCUCCUUUACGAAUACUUCACUGUCUACAACGAACUGACUAAAGU |
| | | GAAAUACGUUACUGAAGGAAUGAGGAAGCCGGCCUUUCUGUCCG |
| | | GAGAACAGAAGAAAGCAAUUGUCGAUCUGCUGUUCAAGACCAAC |
| | | CGCAAGGUGACCGUCAAGCAGCUUAAAGAGGACUACUUCAAGAA |
| | | GAUCGAGUGUUUCGACUCAGUGGAAAUCAGCGGGGUGGAGGACA |
| | | GAUUCAACGCUUCGCUGGGAACCUAUCAUGAUCUCCUGAAGAUC |
| | | AUCAAGGACAAGGACUUCCUUGACAACGAGGAGAACGAGGACAU |
| | | CCUGGAAGAUAUCGUCCUGACCUUGACCCUUUUCGAGGAUCGCGA |
| | | GAUGAUCGAGGAGAGGCUUAAGACCUACGCUCAUCUCUUCGACG |
| | | AUAAGGUCAUGAAACAACUCAAGCGCCGCCGGUACACUGGUUGG |
| | | GGCCGCCUCUCCCGCAAGCUGAUCAACGGUAUUCGCGAUAAACAG |
| | | AGCGGUAAAACUAUCCUGGAUUUCCUCAAAUCGGAUGGCUUCGC |
| | | UAAUCGUAACUUCAUGCAAUUGAUCCACGACGACAGCCUGACCUU |
| | | UAAGGAGGACAUCCAAAAAGCACAAGUGUCCGGACAGGGAGACU |
| | | CACUCCAUGAACACAUCGCGAAUCUGGCCGGUUCGCCGGCGAUUA |
| | | AGAAGGGAAUUCUGCAAACUGUGAAGGUGGUCGACGAGCUGGUG |
| | | AAGGUCAUGGGACGGCACAAACCGGAGAAUAUCGUGAUUGAAAU |
| | | GGCCCGAGAAAACCAGACUACCCAGAAGGGCCAGAAAAACUCCCG |
| | | CGAAAGGAUGAAGCGGAUCGAAGAAGGAAUCAAGGAGCUGGGCA |
| | | GCCAGAUCCUGAAAGAGCACCCGGUGGAAAACACGCAGCUGCAGA |
| | | ACGAGAAGCUCUACCUGUACUAUUUGCAAAAUGGACGGGACAUG |
| | | UACGUGGACCAAGAGCUGGACAUCAAUCGGUUGUCUGAUUACGA |
| | | CGUGGACCACAUCGUUCCACAGUCCUUUCUGAAGGAUGACUCGAU |
| | | CGAUAACAAGGUGUUGACUCGCAGCGACAAGAACAGAGGGAAGU |
| | | CAGAUAAUGUGCCAUCGGAGGAGGUCGUGAAGAAGAUGAAGAAU |
| | | UACUGGCGGCAGCUCCUGAAUGCGAAGCUGAUUACCCAGAGAAA |
| | | GUUUGACAAUCUCACUAAAGCCGAGCGCGGCGGACUCUCAGAGCU |
| | | GGAUAAGGCUGGAUUCAUCAAACGGCAGCUGGUCGAGACUCGGC |
| | | AGAUUACCAAGCACGUGGCGCAGAUCUUGGACUCCCGCAUGAACA |
| | | CUAAAUACGACGAGAACGAUAAGCUCAUCCGGGAAGUGAAGGUG |
| | | AUUACCCUGAAAAGCAAACUUGUGUCGGACUUUCGGAAGGACUU |
| | | UCAGUUUUACAAAGUGAGAGAAAUCAACAACUACCAUCACGCGC |
| | | AUGACGCAUACCUCAACGCUGUGGUCGGUACCGCCCUGAUCAAAA |
| | | AGUACCCUAAACUUGAAUCGGAGUUUGUGUACGGAGACUACAAG |
| | | GUCUACGACGUGAGGAAGAUGAUAGCCAAGUCCGAACAGGAAAU |
| | | CGGGAAAGCAACUGCGAAAUACUUCUUUUACUCAAACAUCAUGA |
| | | ACUUUUUCAAGACUGAAAUUACGCUGGCCAAUGGAGAAAUCAGG |
| | | AAGAGGCCACUGAUCGAAACUAACGGAGAAACGGGCGAAAUCGU |
| | | GUGGGACAAGGGCAGGGACUUCGCAACUGUUCGCAAAGUGCUCU |
| | | CUAUGCCGCAAGUCAAUAUUGUGAAGAAAACCGAAGUGCAAACC |
| | | GGCGGAUUUUCAAAGGAAUCGAUCCUCCCAAAGAGAAAUAGCGA |
| | | CAAGCUCAUUGCACGCAAGAAAGACUGGGACCCGAAGAAGUACG |
| | | GAGGAUUCGAUUCGCCGACUGUCGCAUACUCCGUCCUCGUGGUGG |
| | | CCAAGGUGGAGAAGGGAAAGAGCAAAAAGCUCAAAUCCGUCAAA |
| | | GAGCUGCUGGGGAUUACCAUCAUGGAACGAUCCUCGUUCGAGAA |
| | | GAACCCGAUUGAUUUCCUCGAGGCGAAGGGUUACAAGGAGGUGA |
| | | AGAAGGAUCUGAUCAUCAAACUCCCCAAGUACUCACUGUUCGAAC |
| | | UGGAAAAUGGUCGGAAGCGCAUGCUGGCUUCGGCCGGAGAACUC |
| | | CAAAAAGGAAAUGAGCUGGCCUUGCCUAGCAAGUACGUCAACUU |
| | | CCUCUAUCUUGCUUCGCACUACGAAAAACUCAAAGGGUCACCGGA |
| | | AGAUAACGAACAGAAGCAGCUUUUCGUGGAGCAGCACAAGCAUU |
| | | AUCUGGAUGAAAUCAUCGAACAAAUCUCCGAGUUUUCAAAGCGC |
| | | GUGAUCCUCGCCGACGCCAACCUCGACAAAGUCCUGUCGGCCUAC |
| | | AAUAAGCAUAGAGAUAAGCCGAUCAGAGAACAGGCCGAGAACAU |
| | | UAUCCACUUGUUCACCCUGACUAACCUGGGAGCCCCAGCCGCCUU |
| | | CAAGUACUUCGAUACUACUAUCGAUCGCAAAAGAUACACGUCCAC |
| | | CAAGGAAGUUCUGGACGCGACCCUGAUCCACCAAAGCAUCACUGG |
| | | ACUCUACGAAACUAGGAUCGAUCUGUCGCAGCUGGGUGGCGAUU |
| | | CUGGUGGUUCUACUAAUCUGUCAGAUAUUAUUGAAAAGGAGACC |
| | | GGUAAGCAACUGGUUAUCCAGGAAUCCAUCCUCAUGCUCCCAGAG |
| | | GAGGUGGAAGAAGUCAUUGGGAACAAGCCGGAAAGCGAUAUACU |
| | | CGUGCACACCGCCUACGACGAGAGCACCGACGAGAAUGUCAUGCU |
| | | UCUGACUAGCGACGCCCCUGAAUACAAGCCUUGGGCUCUGGUCAU |
| | | ACAGGAUAGCAACGGUGAGAACAAGAUUAAGAUGCUCUCUGGUG |
| | | GUUCUCCCAAGAAGAAGAGGAAAGUCUAA |
| 12 | Amino acid | MSSETGPVAVDPTLRRRIEPHEFEVFFDPRELRKETCLLYEINWGGRHSI |
| | sequence for BE3 | WRHTSQNTNKHVEVNFIEKFTTERYFCPNTRCSITWFLSWSPCGECSRA |
| | | ITEFLSRYPHVTLFIYIARLYHHADPRNRQGLRDLISSGVTIQIMTEQESG |
| | | YCWRNFVNYSPSNEAHWPRYPHLWVRLYVLELYCIILGLPPCLNILRR |
| | | KQPQLTFFTIALQSCHYQRLPPHILWATGLKSGSETPGTSESATPESDKK |
| | | YSIGLAIGTNSVGWAVITDEYKVPSKKFKVLGNTDRHSIKKNLIGALLF |
| | | DSGETAEATRLKRTARRRYTRRKNRICYLQEIFSNEMAKVDDSFFHRL |
| | | EESFLVEEDKKHERHPIFGNIVDEVAYHEKYPTIYHLRKKLVDSTDKAD |
| | | LRLIYLALAHMIKFRGHFLIEGDLNPDNSDVDKLFIQLVQTYNQLFEEN |
| | | PINASGVDAKAILSARLSKSRRLENLIAQLPGEKKNGLFGNLIALSLGLT |
| | | PNFKSNFDLAEDAKLQLSKDTYDDDLDNLLAQIGDQYADLFLAAKNL |
| | | SDAILLSDILRVNTEITKAPLSASMIKRYDEHHQDLTLLKALVRQQLPE |
| | | KYKEIFFDQSKNGYAGYIDGGASQEEFYKFIKPILEKMDGTEELLVKLN |
| | | REDLLRKQRTFDNGSIPHQIHLGELHAILRRQEDFYPFLKDNREKIEKIL |
| | | TFRIPYYVGPLARGNSRFAWMTRKSEETITPWNFEEVVDKGASAQSFIE |
| | | RMTNFDKNLPNEKVLPKHSLLYEYFTVYNELTKVKYVTEGMRKPAFL |
| | | SGEQKKAIVDLLFKTNRKVTVKQLKEDYFKKIECFDSVEISGVEDRFNA |
| | | SLGTYHDLLKIIKDKDFLDNEENEDILEDIVLTLTLFEDREMIEERLKTY |
| | | AHLFDDKVMKQLKRRRYTGWGRLSRKLINGIRDKQSGKTILDFLKSD |
| | | GFANRNFMQLIHDDSLTFKEDIQKAQVSGQGDSLHEHIANLAGSPAIK |
| | | KGILQTVKVVDELVKVMGRHKPENIVIEMARENQTTQKGQKNSRERM |
| | | KRIEEGIKELGSQILKEHPVENTQLQNEKLYLYYLQNGRDMYVDQELD |
| | | INRLSDYDVDHIVPQSFLKDDSIDNKVLTRSDKNRGKSDNVPSEEVVK |
| | | KMKNYWRQLLNAKLITQRKFDNLTKAERGGLSELDKAGFIKRQLVET |
| | | RQITKHVAQILDSRMNTKYDENDKLIREVKVITLKSKLVSDFRKDFQFY |
| | | KVREINNYHHAHDAYLNAVVGTALIKKYPKLESEFVYGDYKVYDVRK |
| | | MIAKSEQEIGKATAKYFFYSNIMNFFKTEITLANGEIRKRPLIETNGETG |
| | | EIVWDKGRDFATVRKVLSMPQVNIVKKTEVQTGGFSKESILPKRNSDK |
| | | LIARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRMLA |
| | | SAGELQKGNELALPSKYVNFLYLASHYEKLKGSPEDNEQKQLFVEQH |
| | | KHYLDEIIEQISEFSKRVILADANLDKVLSAYNKHRDKPIREQAENIIHL |
| | | FTLTNLGAPAAFKYFDTTIDRKRYTSTKEVLDATLIHQSITGLYETRIDL |
| | | SQLGGDSGGSTNLSDIIEKETGKQLVIQESILMLPEEVEEVIGNKPESDIL |
| | | VHTAYDESTDENVMLLTSDAPEYKPWALVIQDSNGENKIKMLSGGSP |
| | | KKKRKV |
| 13 | mRNA encoding | GGGAGACCCAAGCUGGCUAGCUCCCGCAGUCGGCGUCCAGCGGCU |
| | UGI | CUGCUUGUUCGUGUGUGUGUCGUUGCAGGCCUUAUUCGGAUCCG |
| | | CCACCAUGGGACCGAAGAAGAAGAGAAAGGUCGGAGGAGGAAGC |
| | | ACAAACCUGUCGGACAUCAUCGAAAAGGAAACAGGAAAGCAGCU |
| | | GGUCAUCCAGGAAUCGAUCCUGAUGCUGCCGGAAGAAGUCGAAG |
| | | AAGUCAUCGGAAACAAGCCGGAAUCGGACAUCCUGGUCCACACAG |
| | | CAUACGACGAAUCGACAGACGAAAACGUCAUGCUGCUGACAUCG |
| | | GACGCACCGGAAUACAAGCCGUGGGCACUGGUCAUCCAGGACUCG |
| | | AACGGAGAAAACAAGAUCAAGAUGCUGUGAUAGUCUAGACAUCA |
| | | CAUUUAAAAGCAUCUCAGCCUACCAUGAGAAUAAGAGAAAGAAA |
| | | AUGAAGAUCAAUAGCUUAUUCAUCUCUUUUUCUUUUUCGUUGGU |
| | | GUAAAGCCAACACCCUGUCUAAAAAACAUAAAUUUCUUUAAUCA |
| | | UUUUGCCUCUUUUCUCUGUGCUUCAAUUAAUAAAAAAUGGAAAG |
| | | AACCUCGAGUCUAG |
| 14 | Open reading | AUGGGACCGAAGAAGAAGAGAAAGGUCGGAGGAGGAAGCACAAA |
| | frame for UGI | CCUGUCGGACAUCAUCGAAAAGGAAACAGGAAAGCAGCUGGUCA |
| | | UCCAGGAAUCGAUCCUGAUGCUGCCGGAAGAAGUCGAAGAAGUC |
| | | AUCGGAAACAAGCCGGAAUCGGACAUCCUGGUCCACACAGCAUAC |
| | | GACGAAUCGACAGACGAAAACGUCAUGCUGCUGACAUCGGACGC |
| | | ACCGGAAUACAAGCCGUGGGCACUGGUCAUCCAGGACUCGAACGG |
| | | AGAAAACAAGAUCAAGAUGCUGUGA |
| 15 | Amino acid | MTNLSDIIEKETGKQLVIQESILMLPEEVEEVIGNKPESDILVHTAYDES |
| | sequence for UGI | TDENVMLLTSDAPEYKPWALVIQDSNGENKIKMLSGGSKRTADGSEFE |
| | | SPKKKRKVE |
| 16 | mRNA encoding | GGGAAGCUCAGAAUAAACGCUCAACUUUGGCCGGAUCUGCCACCA |
| | BC22 with 2x | UGGAGGCCUCCCCCGCCUCCGGCCCCCGGCACCUGAUGGACCCCC |
| | UGI | ACAUCUUCACCUCCAACUUCAACAACGGCAUCGGCCGGCACAAGA |
| | | CCUACCUGUGCUACGAGGUGGAGCGGCUGGACAACGGCACCUCCG |
| | | UGAAGAUGGACCAGCACCGGGGCUUCCUGCACAACCAGGCCAAGA |
| | | ACCUGCUGUGCGGCUUCUACGGCCGGCACGCCGAGCUGCGGUUCC |
| | | UGGACCUGGUGCCCUCCCUGCAGCUGGACCCCGCCCAGAUCUACC |
| | | GGGUGACCUGGUUCAUCUCCUGGUCCCCCUGCUUCUCCUGGGGCU |
| | | GCGCCGGCGAGGUGCGGGCCUUCCUGCAGGAGAACACCCACGUGC |
| | | GGCUGCGGAUCUUCGCCGCCCGGAUCUACGACUACGACCCCCUGU |
| | | ACAAGGAGGCCCUGCAGAUGCUGCGGGACGCCGGCGCCCAGGUGU |
| | | CCAUCAUGACCUACGACGAGUUCAAGCACUGCUGGGACACCUUCG |
| | | UGGACCACCAGGGCUGCCCCUUCCAGCCCUGGGACGGCCUGGACG |
| | | AGCACUCCCAGGCCCUGUCCGGCCGGCUGCGGGCCAUCCUGCAGA |
| | | ACCAGGGCAACUCCGGCUCCGAGACCCCCGGCACCUCCGAGUCCG |
| | | CCACCCCCGAGUCCGACAAGAAGUACUCCAUCGGCCUGGCCAUCG |
| | | GCACCAACUCCGUGGGCUGGGCCGUGAUCACCGACGAGUACAAGG |
| | | UGCCCUCCAAGAAGUUCAAGGUGCUGGGCAACACCGACCGGCACU |
| | | CCAUCAAGAAGAACCUGAUCGGCGCCCUGCUGUUCGACUCCGGCG |
| | | AGACCGCCGAGGCCACCCGGCUGAAGCGGACCGCCCGGCGGCGGU |
| | | ACACCCGGCGGAAGAACCGGAUCUGCUACCUGCAGGAGAUCUUCU |
| | | CCAACGAGAUGGCCAAGGUGGACGACUCCUUCUUCCACCGGCUGG |
| | | AGGAGUCCUUCCUGGUGGAGGAGGACAAGAAGCACGAGCGGCAC |
| | | CCCAUCUUCGGCAACAUCGUGGACGAGGUGGCCUACCACGAGAAG |
| | | UACCCCACCAUCUACCACCUGCGGAAGAAGCUGGUGGACUCCACC |
| | | GACAAGGCCGACCUGCGGCUGAUCUACCUGGCCCUGGCCCACAUG |
| | | AUCAAGUUCCGGGGCCACUUCCUGAUCGAGGGCGACCUGAACCCC |
| | | GACAACUCCGACGUGGACAAGCUGUUCAUCCAGCUGGUGCAGACC |
| | | UACAACCAGCUGUUCGAGGAGAACCCCAUCAACGCCUCCGGCGUG |
| | | GACGCCAAGGCCAUCCUGUCCGCCCGGCUGUCCAAGUCCCGGCGG |
| | | CUGGAGAACCUGAUCGCCCAGCUGCCCGGCGAGAAGAAGAACGGC |
| | | CUGUUCGGCAACCUGAUCGCCCUGUCCCUGGGCCUGACCCCCAAC |
| | | UUCAAGUCCAACUUCGACCUGGCCGAGGACGCCAAGCUGCAGCUG |
| | | UCCAAGGACACCUACGACGACGACCUGGACAACCUGCUGGCCCAG |
| | | AUCGGCGACCAGUACGCCGACCUGUUCCUGGCCGCCAAGAACCUG |
| | | UCCGACGCCAUCCUGCUGUCCGACAUCCUGCGGGUGAACACCGAG |
| | | AUCACCAAGGCCCCCCUGUCCGCCUCCAUGAUCAAGCGGUACGAC |
| | | GAGCACCACCAGGACCUGACCCUGCUGAAGGCCCUGGUGCGGCAG |
| | | CAGCUGCCCGAGAAGUACAAGGAGAUCUUCUUCGACCAGUCCAAG |
| | | AACGGCUACGCCGGCUACAUCGACGGCGGCGCCUCCCAGGAGGAG |
| | | UUCUACAAGUUCAUCAAGCCCAUCCUGGAGAAGAUGGACGGCACC |
| | | GAGGAGCUGCUGGUGAAGCUGAACCGGGAGGACCUGCUGCGGAA |
| | | GCAGCGGACCUUCGACAACGGCUCCAUCCCCCACCAGAUCCACCU |
| | | GGGCGAGCUGCACGCCAUCCUGCGGCGGCAGGAGGACUUCUACCC |
| | | CUUCCUGAAGGACAACCGGGAGAAGAUCGAGAAGAUCCUGACCU |
| | | UCCGGAUCCCCUACUACGUGGGCCCCCUGGCCCGGGGCAACUCCC |
| | | GGUUCGCCUGGAUGACCCGGAAGUCCGAGGAGACCAUCACCCCCU |
| | | GGAACUUCGAGGAGGUGGUGGACAAGGGCGCCUCCGCCCAGUCCU |
| | | UCAUCGAGCGGAUGACCAACUUCGACAAGAACCUGCCCAACGAGA |
| | | AGGUGCUGCCCAAGCACUCCCUGCUGUACGAGUACUUCACCGUGU |
| | | ACAACGAGCUGACCAAGGUGAAGUACGUGACCGAGGGCAUGCGG |
| | | AAGCCCGCCUUCCUGUCCGGCGAGCAGAAGAAGGCCAUCGUGGAC |
| | | CUGCUGUUCAAGACCAACCGGAAGGUGACCGUGAAGCAGCUGAA |
| | | GGAGGACUACUUCAAGAAGAUCGAGUGCUUCGACUCCGUGGAGA |
| | | UCUCCGGCGUGGAGGACCGGUUCAACGCCUCCCUGGGCACCUACC |
| | | ACGACCUGCUGAAGAUCAUCAAGGACAAGGACUUCCUGGACAAC |
| | | GAGGAGAACGAGGACAUCCUGGAGGACAUCGUGCUGACCCUGAC |
| | | CCUGUUCGAGGACCGGGAGAUGAUCGAGGAGCGGCUGAAGACCU |
| | | ACGCCCACCUGUUCGACGACAAGGUGAUGAAGCAGCUGAAGCGGC |
| | | GGCGGUACACCGGCUGGGGCCGGCUGUCCCGGAAGCUGAUCAACG |
| | | GCAUCCGGGACAAGCAGUCCGGCAAGACCAUCCUGGACUUCCUGA |
| | | AGUCCGACGGCUUCGCCAACCGGAACUUCAUGCAGCUGAUCCACG |
| | | ACGACUCCCUGACCUUCAAGGAGGACAUCCAGAAGGCCCAGGUGU |
| | | CCGGCCAGGGCGACUCCCUGCACGAGCACAUCGCCAACCUGGCCG |
| | | GCUCCCCCGCCAUCAAGAAGGGCAUCCUGCAGACCGUGAAGGUGG |
| | | UGGACGAGCUGGUGAAGGUGAUGGGCCGGCACAAGCCCGAGAAC |
| | | AUCGUGAUCGAGAUGGCCCGGGAGAACCAGACCACCCAGAAGGGC |
| | | CAGAAGAACUCCCGGGAGCGGAUGAAGCGGAUCGAGGAGGGCAU |
| | | CAAGGAGCUGGGCUCCCAGAUCCUGAAGGAGCACCCCGUGGAGAA |
| | | CACCCAGCUGCAGAACGAGAAGCUGUACCUGUACUACCUGCAGAA |
| | | CGGCCGGGACAUGUACGUGGACCAGGAGCUGGACAUCAACCGGCU |
| | | GUCCGACUACGACGUGGACCACAUCGUGCCCCAGUCCUUCCUGAA |
| | | GGACGACUCCAUCGACAACAAGGUGCUGACCCGGUCCGACAAGAA |
| | | CCGGGGCAAGUCCGACAACGUGCCCUCCGAGGAGGUGGUGAAGA |
| | | AGAUGAAGAACUACUGGCGGCAGCUGCUGAACGCCAAGCUGAUC |
| | | ACCCAGCGGAAGUUCGACAACCUGACCAAGGCCGAGCGGGGCGGC |
| | | CUGUCCGAGCUGGACAAGGCCGGCUUCAUCAAGCGGCAGCUGGUG |
| | | GAGACCCGGCAGAUCACCAAGCACGUGGCCCAGAUCCUGGACUCC |
| | | CGGAUGAACACCAAGUACGACGAGAACGACAAGCUGAUCCGGGA |
| | | GGUGAAGGUGAUCACCCUGAAGUCCAAGCUGGUGUCCGACUUCC |
| | | GGAAGGACUUCCAGUUCUACAAGGUGCGGGAGAUCAACAACUAC |
| | | CACCACGCCCACGACGCCUACCUGAACGCCGUGGUGGGCACCGCC |
| | | CUGAUCAAGAAGUACCCCAAGCUGGAGUCCGAGUUCGUGUACGG |
| | | CGACUACAAGGUGUACGACGUGCGGAAGAUGAUCGCCAAGUCCG |
| | | AGCAGGAGAUCGGCAAGGCCACCGCCAAGUACUUCUUCUACUCCA |
| | | ACAUCAUGAACUUCUUCAAGACCGAGAUCACCCUGGCCAACGGCG |
| | | AGAUCCGGAAGCGGCCCCUGAUCGAGACCAACGGCGAGACCGGCG |
| | | AGAUCGUGUGGGACAAGGGCCGGGACUUCGCCACCGUGCGGAAG |
| | | GUGCUGUCCAUGCCCCAGGUGAACAUCGUGAAGAAGACCGAGGU |
| | | GCAGACCGGCGGCUUCUCCAAGGAGUCCAUCCUGCCCAAGCGGAA |
| | | CUCCGACAAGCUGAUCGCCCGGAAGAAGGACUGGGACCCCAAGAA |
| | | GUACGGCGGCUUCGACUCCCCCACCGUGGCCUACUCCGUGCUGGU |
| | | GGUGGCCAAGGUGGAGAAGGGCAAGUCCAAGAAGCUGAAGUCCG |
| | | UGAAGGAGCUGCUGGGCAUCACCAUCAUGGAGCGGUCCUCCUUCG |
| | | AGAAGAACCCCAUCGACUUCCUGGAGGCCAAGGGCUACAAGGAG |
| | | GUGAAGAAGGACCUGAUCAUCAAGCUGCCCAAGUACUCCCUGUUC |
| | | GAGCUGGAGAACGGCCGGAAGCGGAUGCUGGCCUCCGCCGGCGAG |
| | | CUGCAGAAGGGCAACGAGCUGGCCCUGCCCUCCAAGUACGUGAAC |
| | | UUCCUGUACCUGGCCUCCCACUACGAGAAGCUGAAGGGCUCCCCC |
| | | GAGGACAACGAGCAGAAGCAGCUGUUCGUGGAGCAGCACAAGCA |
| | | CUACCUGGACGAGAUCAUCGAGCAGAUCUCCGAGUUCUCCAAGCG |
| | | GGUGAUCCUGGCCGACGCCAACCUGGACAAGGUGCUGUCCGCCUA |
| | | CAACAAGCACCGGGACAAGCCCAUCCGGGAGCAGGCCGAGAACAU |
| | | CAUCCACCUGUUCACCCUGACCAACCUGGGCGCCCCCGCCGCCUU |
| | | CAAGUACUUCGACACCACCAUCGACCGGAAGCGGUACACCUCCAC |
| | | CAAGGAGGUGCUGGACGCCACCCUGAUCCACCAGUCCAUCACCGG |
| | | CCUGUACGAGACCCGGAUCGACCUGUCCCAGCUGGGCGGCGACUC |
| | | CGGCGGCUCCGGCGGCUCCGGCGGCUCCACCAACCUGUCCGACAU |
| | | CAUCGAGAAGGAGACCGGCAAGCAGCUGGUGAUCCAGGAGUCCA |
| | | UCCUGAUGCUGCCCGAGGAGGUGGAGGAGGUGAUCGGCAACAAG |
| | | CCCGAGUCCGACAUCCUGGUGCACACCGCCUACGACGAGUCCACC |
| | | GACGAGAACGUGAUGCUGCUGACCUCCGACGCCCCCGAGUACAAG |
| | | CCCUGGGCCCUGGUGAUCCAGGACUCCAACGGCGAGAACAAGAUC |
| | | AAGAUGCUGUCCGGCGGCUCCGGCGGCUCCGGCGGCUCCACCAAC |
| | | CUGUCCGACAUCAUCGAGAAGGAGACCGGCAAGCAGCUGGUGAU |
| | | CCAGGAGUCCAUCCUGAUGCUGCCCGAGGAGGUGGAGGAGGUGA |
| | | UCGGCAACAAGCCCGAGUCCGACAUCCUGGUGCACACCGCCUACG |
| | | ACGAGUCCACCGACGAGAACGUGAUGCUGCUGACCUCCGACGCCC |
| | | CCGAGUACAAGCCCUGGGCCCUGGUGAUCCAGGACUCCAACGGCG |
| | | AGAACAAGAUCAAGAUGCUGUCCGGCGGCUCCAAGCGGACCGCCG |
| | | ACGGCUCCGAGUUCGAGCCCAAGAAGAAGCGGAAGGUGUGAUAG |
| | | CUAGCACCAGCCUCAAGAACACCCGAAUGGAGUCUCUAAGCUACA |
| | | UAAUACCAACUUACACUUUACAAAAUGUUGUCCCCCAAAAUGUA |
| | | GCCAUUCGUAUCUGCUCCUAAUAAAAAGAAAGUUUCUUCACAUU |
| | | CUCUCGAGAAAAAAAAAAAAUGGAAAAAAAAAAAACGGAAAAAA |
| | | AAAAAAGGUAAAAAAAAAAAAUAUAAAAAAAAAAAACAUAAAAA |
| | | AAAAAAACGAAAAAAAAAAAACGUAAAAAAAAAAAACUCAAAAA |
| | | AAAAAAAGAUAAAAAAAAAAAACCUAAAAAAAAAAAAUGUAAAA |
| | | AAAAAAAAGGGAAAAAAAAAAAACGCAAAAAAAAAAAACACAAA |
| | | AAAAAAAAAUGCAAAAAAAAAAAAUCGAAAAAAAAAAAAUCUAA |
| | | AAAAAAAAAACGAAAAAAAAAAAACCCAAAAAAAAAAAAGACAA |
| | | AAAAAAAAAAUAGAAAAAAAAAAAAGUUAAAAAAAAAAAACUGA |
| | | AAAAAAAAAAAUUUAAAAAAAAAAAAUCUAG |
| 17 | Open reading | AUGGAGGCCUCCCCCGCCUCCGGCCCCCGGCACCUGAUGGACCCC |
| | frame for BC22 | CACAUCUUCACCUCCAACUUCAACAACGGCAUCGGCCGGCACAAG |
| | with 2x UGI | ACCUACCUGUGCUACGAGGUGGAGCGGCUGGACAACGGCACCUCC |
| | | GUGAAGAUGGACCAGCACCGGGGCUUCCUGCACAACCAGGCCAAG |
| | | AACCUGCUGUGCGGCUUCUACGGCCGGCACGCCGAGCUGCGGUUC |
| | | CUGGACCUGGUGCCCUCCCUGCAGCUGGACCCCGCCCAGAUCUAC |
| | | CGGGUGACCUGGUUCAUCUCCUGGUCCCCCUGCUUCUCCUGGGGC |
| | | UGCGCCGGCGAGGUGCGGGCCUUCCUGCAGGAGAACACCCACGUG |
| | | CGGCUGCGGAUCUUCGCCGCCCGGAUCUACGACUACGACCCCCUG |
| | | UACAAGGAGGCCCUGCAGAUGCUGCGGGACGCCGGCGCCCAGGUG |
| | | UCCAUCAUGACCUACGACGAGUUCAAGCACUGCUGGGACACCUUC |
| | | GUGGACCACCAGGGCUGCCCCUUCCAGCCCUGGGACGGCCUGGAC |
| | | GAGCACUCCCAGGCCCUGUCCGGCCGGCUGCGGGCCAUCCUGCAG |
| | | AACCAGGGCAACUCCGGCUCCGAGACCCCCGGCACCUCCGAGUCC |
| | | GCCACCCCCGAGUCCGACAAGAAGUACUCCAUCGGCCUGGCCAUC |
| | | GGCACCAACUCCGUGGGCUGGGCCGUGAUCACCGACGAGUACAAG |
| | | GUGCCCUCCAAGAAGUUCAAGGUGCUGGGCAACACCGACCGGCAC |
| | | UCCAUCAAGAAGAACCUGAUCGGCGCCCUGCUGUUCGACUCCGGC |
| | | GAGACCGCCGAGGCCACCCGGCUGAAGCGGACCGCCCGGCGGCGG |
| | | UACACCCGGCGGAAGAACCGGAUCUGCUACCUGCAGGAGAUCUUC |
| | | UCCAACGAGAUGGCCAAGGUGGACGACUCCUUCUUCCACCGGCUG |
| | | GAGGAGUCCUUCCUGGUGGAGGAGGACAAGAAGCACGAGCGGCA |
| | | CCCCAUCUUCGGCAACAUCGUGGACGAGGUGGCCUACCACGAGAA |
| | | GUACCCCACCAUCUACCACCUGCGGAAGAAGCUGGUGGACUCCAC |
| | | CGACAAGGCCGACCUGCGGCUGAUCUACCUGGCCCUGGCCCACAU |
| | | GAUCAAGUUCCGGGGCCACUUCCUGAUCGAGGGCGACCUGAACCC |
| | | CGACAACUCCGACGUGGACAAGCUGUUCAUCCAGCUGGUGCAGAC |
| | | CUACAACCAGCUGUUCGAGGAGAACCCCAUCAACGCCUCCGGCGU |
| | | GGACGCCAAGGCCAUCCUGUCCGCCCGGCUGUCCAAGUCCCGGCG |
| | | GCUGGAGAACCUGAUCGCCCAGCUGCCCGGCGAGAAGAAGAACGG |
| | | CCUGUUCGGCAACCUGAUCGCCCUGUCCCUGGGCCUGACCCCCAA |
| | | CUUCAAGUCCAACUUCGACCUGGCCGAGGACGCCAAGCUGCAGCU |
| | | GUCCAAGGACACCUACGACGACGACCUGGACAACCUGCUGGCCCA |
| | | GAUCGGCGACCAGUACGCCGACCUGUUCCUGGCCGCCAAGAACCU |
| | | GUCCGACGCCAUCCUGCUGUCCGACAUCCUGCGGGUGAACACCGA |
| | | GAUCACCAAGGCCCCCCUGUCCGCCUCCAUGAUCAAGCGGUACGA |
| | | CGAGCACCACCAGGACCUGACCCUGCUGAAGGCCCUGGUGCGGCA |
| | | GCAGCUGCCCGAGAAGUACAAGGAGAUCUUCUUCGACCAGUCCAA |
| | | GAACGGCUACGCCGGCUACAUCGACGGCGGCGCCUCCCAGGAGGA |
| | | GUUCUACAAGUUCAUCAAGCCCAUCCUGGAGAAGAUGGACGGCA |
| | | CCGAGGAGCUGCUGGUGAAGCUGAACCGGGAGGACCUGCUGCGG |
| | | AAGCAGCGGACCUUCGACAACGGCUCCAUCCCCCACCAGAUCCAC |
| | | CUGGGCGAGCUGCACGCCAUCCUGCGGCGGCAGGAGGACUUCUAC |
| | | CCCUUCCUGAAGGACAACCGGGAGAAGAUCGAGAAGAUCCUGACC |
| | | UUCCGGAUCCCCUACUACGUGGGCCCCCUGGCCCGGGGCAACUCC |
| | | CGGUUCGCCUGGAUGACCCGGAAGUCCGAGGAGACCAUCACCCCC |
| | | UGGAACUUCGAGGAGGUGGUGGACAAGGGCGCCUCCGCCCAGUCC |
| | | UUCAUCGAGCGGAUGACCAACUUCGACAAGAACCUGCCCAACGAG |
| | | AAGGUGCUGCCCAAGCACUCCCUGCUGUACGAGUACUUCACCGUG |
| | | UACAACGAGCUGACCAAGGUGAAGUACGUGACCGAGGGCAUGCG |
| | | GAAGCCCGCCUUCCUGUCCGGCGAGCAGAAGAAGGCCAUCGUGGA |
| | | CCUGCUGUUCAAGACCAACCGGAAGGUGACCGUGAAGCAGCUGA |
| | | AGGAGGACUACUUCAAGAAGAUCGAGUGCUUCGACUCCGUGGAG |
| | | AUCUCCGGCGUGGAGGACCGGUUCAACGCCUCCCUGGGCACCUAC |
| | | CACGACCUGCUGAAGAUCAUCAAGGACAAGGACUUCCUGGACAAC |
| | | GAGGAGAACGAGGACAUCCUGGAGGACAUCGUGCUGACCCUGAC |
| | | CCUGUUCGAGGACCGGGAGAUGAUCGAGGAGCGGCUGAAGACCU |
| | | ACGCCCACCUGUUCGACGACAAGGUGAUGAAGCAGCUGAAGCGGC |
| | | GGCGGUACACCGGCUGGGGCCGGCUGUCCCGGAAGCUGAUCAACG |
| | | GCAUCCGGGACAAGCAGUCCGGCAAGACCAUCCUGGACUUCCUGA |
| | | AGUCCGACGGCUUCGCCAACCGGAACUUCAUGCAGCUGAUCCACG |
| | | ACGACUCCCUGACCUUCAAGGAGGACAUCCAGAAGGCCCAGGUGU |
| | | CCGGCCAGGGCGACUCCCUGCACGAGCACAUCGCCAACCUGGCCG |
| | | GCUCCCCCGCCAUCAAGAAGGGCAUCCUGCAGACCGUGAAGGUGG |
| | | UGGACGAGCUGGUGAAGGUGAUGGGCCGGCACAAGCCCGAGAAC |
| | | AUCGUGAUCGAGAUGGCCCGGGAGAACCAGACCACCCAGAAGGGC |
| | | CAGAAGAACUCCCGGGAGCGGAUGAAGCGGAUCGAGGAGGGCAU |
| | | CAAGGAGCUGGGCUCCCAGAUCCUGAAGGAGCACCCCGUGGAGAA |
| | | CACCCAGCUGCAGAACGAGAAGCUGUACCUGUACUACCUGCAGAA |
| | | CGGCCGGGACAUGUACGUGGACCAGGAGCUGGACAUCAACCGGCU |
| | | GUCCGACUACGACGUGGACCACAUCGUGCCCCAGUCCUUCCUGAA |
| | | GGACGACUCCAUCGACAACAAGGUGCUGACCCGGUCCGACAAGAA |
| | | CCGGGGCAAGUCCGACAACGUGCCCUCCGAGGAGGUGGUGAAGA |
| | | AGAUGAAGAACUACUGGCGGCAGCUGCUGAACGCCAAGCUGAUC |
| | | ACCCAGCGGAAGUUCGACAACCUGACCAAGGCCGAGCGGGGCGGC |
| | | CUGUCCGAGCUGGACAAGGCCGGCUUCAUCAAGCGGCAGCUGGUG |
| | | GAGACCCGGCAGAUCACCAAGCACGUGGCCCAGAUCCUGGACUCC |
| | | CGGAUGAACACCAAGUACGACGAGAACGACAAGCUGAUCCGGGA |
| | | GGUGAAGGUGAUCACCCUGAAGUCCAAGCUGGUGUCCGACUUCC |
| | | GGAAGGACUUCCAGUUCUACAAGGUGCGGGAGAUCAACAACUAC |
| | | CACCACGCCCACGACGCCUACCUGAACGCCGUGGUGGGCACCGCC |
| | | CUGAUCAAGAAGUACCCCAAGCUGGAGUCCGAGUUCGUGUACGG |
| | | CGACUACAAGGUGUACGACGUGCGGAAGAUGAUCGCCAAGUCCG |
| | | AGCAGGAGAUCGGCAAGGCCACCGCCAAGUACUUCUUCUACUCCA |
| | | ACAUCAUGAACUUCUUCAAGACCGAGAUCACCCUGGCCAACGGCG |
| | | AGAUCCGGAAGCGGCCCCUGAUCGAGACCAACGGCGAGACCGGCG |
| | | AGAUCGUGUGGGACAAGGGCCGGGACUUCGCCACCGUGCGGAAG |
| | | GUGCUGUCCAUGCCCCAGGUGAACAUCGUGAAGAAGACCGAGGU |
| | | GCAGACCGGCGGCUUCUCCAAGGAGUCCAUCCUGCCCAAGCGGAA |
| | | CUCCGACAAGCUGAUCGCCCGGAAGAAGGACUGGGACCCCAAGAA |
| | | GUACGGCGGCUUCGACUCCCCCACCGUGGCCUACUCCGUGCUGGU |
| | | GGUGGCCAAGGUGGAGAAGGGCAAGUCCAAGAAGCUGAAGUCCG |
| | | UGAAGGAGCUGCUGGGCAUCACCAUCAUGGAGCGGUCCUCCUUCG |
| | | AGAAGAACCCCAUCGACUUCCUGGAGGCCAAGGGCUACAAGGAG |
| | | GUGAAGAAGGACCUGAUCAUCAAGCUGCCCAAGUACUCCCUGUUC |
| | | GAGCUGGAGAACGGCCGGAAGCGGAUGCUGGCCUCCGCCGGCGAG |
| | | CUGCAGAAGGGCAACGAGCUGGCCCUGCCCUCCAAGUACGUGAAC |
| | | UUCCUGUACCUGGCCUCCCACUACGAGAAGCUGAAGGGCUCCCCC |
| | | GAGGACAACGAGCAGAAGCAGCUGUUCGUGGAGCAGCACAAGCA |
| | | CUACCUGGACGAGAUCAUCGAGCAGAUCUCCGAGUUCUCCAAGCG |
| | | GGUGAUCCUGGCCGACGCCAACCUGGACAAGGUGCUGUCCGCCUA |
| | | CAACAAGCACCGGGACAAGCCCAUCCGGGAGCAGGCCGAGAACAU |
| | | CAUCCACCUGUUCACCCUGACCAACCUGGGCGCCCCCGCCGCCUU |
| | | CAAGUACUUCGACACCACCAUCGACCGGAAGCGGUACACCUCCAC |
| | | CAAGGAGGUGCUGGACGCCACCCUGAUCCACCAGUCCAUCACCGG |
| | | CCUGUACGAGACCCGGAUCGACCUGUCCCAGCUGGGCGGCGACUC |
| | | CGGCGGCUCCGGCGGCUCCGGCGGCUCCACCAACCUGUCCGACAU |
| | | CAUCGAGAAGGAGACCGGCAAGCAGCUGGUGAUCCAGGAGUCCA |
| | | UCCUGAUGCUGCCCGAGGAGGUGGAGGAGGUGAUCGGCAACAAG |
| | | CCCGAGUCCGACAUCCUGGUGCACACCGCCUACGACGAGUCCACC |
| | | GACGAGAACGUGAUGCUGCUGACCUCCGACGCCCCCGAGUACAAG |
| | | CCCUGGGCCCUGGUGAUCCAGGACUCCAACGGCGAGAACAAGAUC |
| | | AAGAUGCUGUCCGGCGGCUCCGGCGGCUCCGGCGGCUCCACCAAC |
| | | CUGUCCGACAUCAUCGAGAAGGAGACCGGCAAGCAGCUGGUGAU |
| | | CCAGGAGUCCAUCCUGAUGCUGCCCGAGGAGGUGGAGGAGGUGA |
| | | UCGGCAACAAGCCCGAGUCCGACAUCCUGGUGCACACCGCCUACG |
| | | ACGAGUCCACCGACGAGAACGUGAUGCUGCUGACCUCCGACGCCC |
| | | CCGAGUACAAGCCCUGGGCCCUGGUGAUCCAGGACUCCAACGGCG |
| | | AGAACAAGAUCAAGAUGCUGUCCGGCGGCUCCAAGCGGACCGCCG |
| | | ACGGCUCCGAGUUCGAGCCCAAGAAGAAGCGGAAGGUGUGAUAG |
| 18 | Amino acid | MEASPASGPRHLMDPHIFTSNFNNGIGRHKTYLCYEVERLDNGTSVKM |
| | sequence for | DQHRGFLHNQAKNLLCGFYGRHAELRFLDLVPSLQLDPAQIYRVTWFI |
| | BC22 with 2x | SWSPCFSWGCAGEVRAFLQENTHVRLRIFAARIYDYDPLYKEALQMLR |
| | UGI | DAGAQVSIMTYDEFKHCWDTFVDHQGCPFQPWDGLDEHSQALSGRL |
| | | RAILQNQGNSGSETPGTSESATPESDKKYSIGLAIGTNSVGWAVITDEY |
| | | KVPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGNIV |
| | | DEVAYHEKYPTIYHLRKKLVDSTDKADLRLIYLALAHMIKFRGHFLIE |
| | | GDLNPDNSDVDKLFIQLVQTYNQLFEENPINASGVDAKAILSARLSKSR |
| | | RLENLIAQLPGEKKNGLFGNLIALSLGLTPNFKSNFDLAEDAKLQLSKD |
| | | TYDDDLDNLLAQIGDQYADLFLAAKNLSDAILLSDILRVNTEITKAPLS |
| | | ASMIKRYDEHHQDLTLLKALVRQQLPEKYKEIFFDQSKNGYAGYIDGG |
| | | ASQEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAWM |
| | | TRKSEETITPWNFEEVVDKGASAQSFIERMTNFDKNLPNEKVLPKHSLL |
| | | YEYFTVYNELTKVKYVTEGMRKPAFLSGEQKKAIVDLLFKTNRKVTV |
| | | KQLKEDYFKKIECFDSVEISGVEDRFNASLGTYHDLLKIIKDKDFLDNE |
| | | ENEDILEDIVLTLTLFEDREMIEERLKTYAHLFDDKVMKQLKRRRYTG |
| | | WGRLSRKLINGIRDKQSGKTILDFLKSDGFANRNFMQLIHDDSLTFKED |
| | | IQKAQVSGQGDSLHEHIANLAGSPAIKKGILQTVKVVDELVKVMGRHK |
| | | PENIVIEMARENQTTQKGQKNSRERMKRIEEGIKELGSQILKEHPVENT |
| | | QLQNEKLYLYYLQNGRDMYVDQELDINRLSDYDVDHIVPQSFLKDDSI |
| | | DNKVLTRSDKNRGKSDNVPSEEVVKKMKNYWRQLLNAKLITQRKFD |
| | | NLTKAERGGLSELDKAGFIKRQLVETRQITKHVAQILDSRMNTKYDEN |
| | | DKLIREVKVITLKSKLVSDFRKDFQFYKVREINNYHHAHDAYLNAVVG |
| | | TALIKKYPKLESEFVYGDYKVYDVRKMIAKSEQEIGKATAKYFFYSNI |
| | | MNFFKTEITLANGEIRKRPLIETNGETGEIVWDKGRDFATVRKVLSMPQ |
| | | VNIVKKTEVQTGGFSKESILPKRNSDKLIARKKDWDPKKYGGFDSPTV |
| | | AYSVLVVAKVEKGKSKKLKSVKELLGITIMERSSFEKNPIDFLEAKGYK |
| | | EVKKDLIIKLPKYSLFELENGRKRMLASAGELQKGNELALPSKYVNFL |
| | | YLASHYEKLKGSPEDNEQKQLFVEQHKHYLDEIIEQISEFSKRVILADA |
| | | NLDKVLSAYNKHRDKPIREQAENIIHLFTLTNLGAPAAFKYFDTTIDRK |
| | | RYTSTKEVLDATLIHQSITGLYETRIDLSQLGGDSGGSGGSGGSTNLSDI |
| | | IEKETGKQLVIQESILMLPEEVEEVIGNKPESDILVHTAYDESTDENVML |
| | | LTSDAPEYKPWALVIQDSNGENKIKMLSGGSGGSGGSTNLSDIIEKETG |
| | | KQLVIQESILMLPEEVEEVIGNKPESDILVHTAYDESTDENVMLLTSDA |
| | | PEYKPWALVIQDSNGENKIKMLSGGSKRTADGSEFEPKKKRKV |
| 19 | mRNA encoding | GGGAAGCUCAGAAUAAACGCUCAACUUUGGCCGGAUCUGCCACCA |
| | BE4MAX protein | UGAAGCGGACCGCCGACGGCUCCGAGUUCGAGUCCCCCAAGAAGA |
| | | AGCGGAAGGUGUCCUCCGAGACCGGCCCCGUGGCCGUGGACCCCA |
| | | CCCUGCGGCGGCGGAUCGAGCCCCACGAGUUCGAGGUGUUCUUCG |
| | | ACCCCCGGGAGCUGCGGAAGGAGACCUGCCUGCUGUACGAGAUCA |
| | | ACUGGGGCGGCCGGCACUCCAUCUGGCGGCACACCUCCCAGAACA |
| | | CCAACAAGCACGUGGAGGUGAACUUCAUCGAGAAGUUCACCACCG |
| | | AGCGGUACUUCUGCCCCAACACCCGGUGCUCCAUCACCUGGUUCC |
| | | UGUCCUGGUCCCCCUGCGGCGAGUGCUCCCGGGCCAUCACCGAGU |
| | | UCCUGUCCCGGUACCCCCACGUGACCCUGUUCAUCUACAUCGCCC |
| | | GGCUGUACCACCACGCCGACCCCCGGAACCGGCAGGGCCUGCGGG |
| | | ACCUGAUCUCCUCCGGCGUGACCAUCCAGAUCAUGACCGAGCAGG |
| | | AGUCCGGCUACUGCUGGCGGAACUUCGUGAACUACUCCCCCUCCA |
| | | ACGAGGCCCACUGGCCCCGGUACCCCCACCUGUGGGUGCGGCUGU |
| | | ACGUGCUGGAGCUGUACUGCAUCAUCCUGGGCCUGCCCCCCUGCC |
| | | UGAACAUCCUGCGGCGGAAGCAGCCCCAGCUGACCUUCUUCACCA |
| | | UCGCCCUGCAGUCCUGCCACUACCAGCGGCUGCCCCCCCACAUCC |
| | | UGUGGGCCACCGGCCUGAAGUCCGGCGGCUCCUCCGGCGGCUCCU |
| | | CCGGCUCCGAGACCCCCGGCACCUCCGAGUCCGCCACCCCCGAGU |
| | | CCUCCGGCGGCUCCUCCGGCGGCUCCGACAAGAAGUACUCCAUCG |
| | | GCCUGGCCAUCGGCACCAACUCCGUGGGCUGGGCCGUGAUCACCG |
| | | ACGAGUACAAGGUGCCCUCCAAGAAGUUCAAGGUGCUGGGCAAC |
| | | ACCGACCGGCACUCCAUCAAGAAGAACCUGAUCGGCGCCCUGCUG |
| | | UUCGACUCCGGCGAGACCGCCGAGGCCACCCGGCUGAAGCGGACC |
| | | GCCCGGCGGCGGUACACCCGGCGGAAGAACCGGAUCUGCUACCUG |
| | | CAGGAGAUCUUCUCCAACGAGAUGGCCAAGGUGGACGACUCCUUC |
| | | UUCCACCGGCUGGAGGAGUCCUUCCUGGUGGAGGAGGACAAGAA |
| | | GCACGAGCGGCACCCCAUCUUCGGCAACAUCGUGGACGAGGUGGC |
| | | CUACCACGAGAAGUACCCCACCAUCUACCACCUGCGGAAGAAGCU |
| | | GGUGGACUCCACCGACAAGGCCGACCUGCGGCUGAUCUACCUGGC |
| | | CCUGGCCCACAUGAUCAAGUUCCGGGGCCACUUCCUGAUCGAGGG |
| | | CGACCUGAACCCCGACAACUCCGACGUGGACAAGCUGUUCAUCCA |
| | | GCUGGUGCAGACCUACAACCAGCUGUUCGAGGAGAACCCCAUCAA |
| | | CGCCUCCGGCGUGGACGCCAAGGCCAUCCUGUCCGCCCGGCUGUC |
| | | CAAGUCCCGGCGGCUGGAGAACCUGAUCGCCCAGCUGCCCGGCGA |
| | | GAAGAAGAACGGCCUGUUCGGCAACCUGAUCGCCCUGUCCCUGGG |
| | | CCUGACCCCCAACUUCAAGUCCAACUUCGACCUGGCCGAGGACGC |
| | | CAAGCUGCAGCUGUCCAAGGACACCUACGACGACGACCUGGACAA |
| | | CCUGCUGGCCCAGAUCGGCGACCAGUACGCCGACCUGUUCCUGGC |
| | | CGCCAAGAACCUGUCCGACGCCAUCCUGCUGUCCGACAUCCUGCG |
| | | GGUGAACACCGAGAUCACCAAGGCCCCCCUGUCCGCCUCCAUGAU |
| | | CAAGCGGUACGACGAGCACCACCAGGACCUGACCCUGCUGAAGGC |
| | | CCUGGUGCGGCAGCAGCUGCCCGAGAAGUACAAGGAGAUCUUCU |
| | | UCGACCAGUCCAAGAACGGCUACGCCGGCUACAUCGACGGCGGCG |
| | | CCUCCCAGGAGGAGUUCUACAAGUUCAUCAAGCCCAUCCUGGAGA |
| | | AGAUGGACGGCACCGAGGAGCUGCUGGUGAAGCUGAACCGGGAG |
| | | GACCUGCUGCGGAAGCAGCGGACCUUCGACAACGGCUCCAUCCCC |
| | | CACCAGAUCCACCUGGGCGAGCUGCACGCCAUCCUGCGGCGGCAG |
| | | GAGGACUUCUACCCCUUCCUGAAGGACAACCGGGAGAAGAUCGA |
| | | GAAGAUCCUGACCUUCCGGAUCCCCUACUACGUGGGCCCCCUGGC |
| | | CCGGGGCAACUCCCGGUUCGCCUGGAUGACCCGGAAGUCCGAGGA |
| | | GACCAUCACCCCCUGGAACUUCGAGGAGGUGGUGGACAAGGGCGC |
| | | CUCCGCCCAGUCCUUCAUCGAGCGGAUGACCAACUUCGACAAGAA |
| | | CCUGCCCAACGAGAAGGUGCUGCCCAAGCACUCCCUGCUGUACGA |
| | | GUACUUCACCGUGUACAACGAGCUGACCAAGGUGAAGUACGUGA |
| | | CCGAGGGCAUGCGGAAGCCCGCCUUCCUGUCCGGCGAGCAGAAGA |
| | | AGGCCAUCGUGGACCUGCUGUUCAAGACCAACCGGAAGGUGACCG |
| | | UGAAGCAGCUGAAGGAGGACUACUUCAAGAAGAUCGAGUGCUUC |
| | | GACUCCGUGGAGAUCUCCGGCGUGGAGGACCGGUUCAACGCCUCC |
| | | CUGGGCACCUACCACGACCUGCUGAAGAUCAUCAAGGACAAGGAC |
| | | UUCCUGGACAACGAGGAGAACGAGGACAUCCUGGAGGACAUCGU |
| | | GCUGACCCUGACCCUGUUCGAGGACCGGGAGAUGAUCGAGGAGC |
| | | GGCUGAAGACCUACGCCCACCUGUUCGACGACAAGGUGAUGAAGC |
| | | AGCUGAAGCGGCGGCGGUACACCGGCUGGGGCCGGCUGUCCCGGA |
| | | AGCUGAUCAACGGCAUCCGGGACAAGCAGUCCGGCAAGACCAUCC |
| | | UGGACUUCCUGAAGUCCGACGGCUUCGCCAACCGGAACUUCAUGC |
| | | AGCUGAUCCACGACGACUCCCUGACCUUCAAGGAGGACAUCCAGA |
| | | AGGCCCAGGUGUCCGGCCAGGGCGACUCCCUGCACGAGCACAUCG |
| | | CCAACCUGGCCGGCUCCCCCGCCAUCAAGAAGGGCAUCCUGCAGA |
| | | CCGUGAAGGUGGUGGACGAGCUGGUGAAGGUGAUGGGCCGGCAC |
| | | AAGCCCGAGAACAUCGUGAUCGAGAUGGCCCGGGAGAACCAGACC |
| | | ACCCAGAAGGGCCAGAAGAACUCCCGGGAGCGGAUGAAGCGGAU |
| | | CGAGGAGGGCAUCAAGGAGCUGGGCUCCCAGAUCCUGAAGGAGC |
| | | ACCCCGUGGAGAACACCCAGCUGCAGAACGAGAAGCUGUACCUGU |
| | | ACUACCUGCAGAACGGCCGGGACAUGUACGUGGACCAGGAGCUG |
| | | GACAUCAACCGGCUGUCCGACUACGACGUGGACCACAUCGUGCCC |
| | | CAGUCCUUCCUGAAGGACGACUCCAUCGACAACAAGGUGCUGACC |
| | | CGGUCCGACAAGAACCGGGGCAAGUCCGACAACGUGCCCUCCGAG |
| | | GAGGUGGUGAAGAAGAUGAAGAACUACUGGCGGCAGCUGCUGAA |
| | | CGCCAAGCUGAUCACCCAGCGGAAGUUCGACAACCUGACCAAGGC |
| | | CGAGCGGGGCGGCCUGUCCGAGCUGGACAAGGCCGGCUUCAUCAA |
| | | GCGGCAGCUGGUGGAGACCCGGCAGAUCACCAAGCACGUGGCCCA |
| | | GAUCCUGGACUCCCGGAUGAACACCAAGUACGACGAGAACGACAA |
| | | GCUGAUCCGGGAGGUGAAGGUGAUCACCCUGAAGUCCAAGCUGG |
| | | UGUCCGACUUCCGGAAGGACUUCCAGUUCUACAAGGUGCGGGAG |
| | | AUCAACAACUACCACCACGCCCACGACGCCUACCUGAACGCCGUG |
| | | GUGGGCACCGCCCUGAUCAAGAAGUACCCCAAGCUGGAGUCCGAG |
| | | UUCGUGUACGGCGACUACAAGGUGUACGACGUGCGGAAGAUGAU |
| | | CGCCAAGUCCGAGCAGGAGAUCGGCAAGGCCACCGCCAAGUACUU |
| | | CUUCUACUCCAACAUCAUGAACUUCUUCAAGACCGAGAUCACCCU |
| | | GGCCAACGGCGAGAUCCGGAAGCGGCCCCUGAUCGAGACCAACGG |
| | | CGAGACCGGCGAGAUCGUGUGGGACAAGGGCCGGGACUUCGCCAC |
| | | CGUGCGGAAGGUGCUGUCCAUGCCCCAGGUGAACAUCGUGAAGA |
| | | AGACCGAGGUGCAGACCGGCGGCUUCUCCAAGGAGUCCAUCCUGC |
| | | CCAAGCGGAACUCCGACAAGCUGAUCGCCCGGAAGAAGGACUGGG |
| | | ACCCCAAGAAGUACGGCGGCUUCGACUCCCCCACCGUGGCCUACU |
| | | CCGUGCUGGUGGUGGCCAAGGUGGAGAAGGGCAAGUCCAAGAAG |
| | | CUGAAGUCCGUGAAGGAGCUGCUGGGCAUCACCAUCAUGGAGCG |
| | | GUCCUCCUUCGAGAAGAACCCCAUCGACUUCCUGGAGGCCAAGGG |
| | | CUACAAGGAGGUGAAGAAGGACCUGAUCAUCAAGCUGCCCAAGU |
| | | ACUCCCUGUUCGAGCUGGAGAACGGCCGGAAGCGGAUGCUGGCCU |
| | | CCGCCGGCGAGCUGCAGAAGGGCAACGAGCUGGCCCUGCCCUCCA |
| | | AGUACGUGAACUUCCUGUACCUGGCCUCCCACUACGAGAAGCUGA |
| | | AGGGCUCCCCCGAGGACAACGAGCAGAAGCAGCUGUUCGUGGAGC |
| | | AGCACAAGCACUACCUGGACGAGAUCAUCGAGCAGAUCUCCGAGU |
| | | UCUCCAAGCGGGUGAUCCUGGCCGACGCCAACCUGGACAAGGUGC |
| | | UGUCCGCCUACAACAAGCACCGGGACAAGCCCAUCCGGGAGCAGG |
| | | CCGAGAACAUCAUCCACCUGUUCACCCUGACCAACCUGGGCGCCC |
| | | CCGCCGCCUUCAAGUACUUCGACACCACCAUCGACCGGAAGCGGU |
| | | ACACCUCCACCAAGGAGGUGCUGGACGCCACCCUGAUCCACCAGU |
| | | CCAUCACCGGCCUGUACGAGACCCGGAUCGACCUGUCCCAGCUGG |
| | | GCGGCGACUCCGGCGGCUCCGGCGGCUCCGGCGGCUCCACCAACC |
| | | UGUCCGACAUCAUCGAGAAGGAGACCGGCAAGCAGCUGGUGAUC |
| | | CAGGAGUCCAUCCUGAUGCUGCCCGAGGAGGUGGAGGAGGUGAU |
| | | CGGCAACAAGCCCGAGUCCGACAUCCUGGUGCACACCGCCUACGA |
| | | CGAGUCCACCGACGAGAACGUGAUGCUGCUGACCUCCGACGCCCC |
| | | CGAGUACAAGCCCUGGGCCCUGGUGAUCCAGGACUCCAACGGCGA |
| | | GAACAAGAUCAAGAUGCUGUCCGGCGGCUCCGGCGGCUCCGGCGG |
| | | CUCCACCAACCUGUCCGACAUCAUCGAGAAGGAGACCGGCAAGCA |
| | | GCUGGUGAUCCAGGAGUCCAUCCUGAUGCUGCCCGAGGAGGUGG |
| | | AGGAGGUGAUCGGCAACAAGCCCGAGUCCGACAUCCUGGUGCACA |
| | | CCGCCUACGACGAGUCCACCGACGAGAACGUGAUGCUGCUGACCU |
| | | CCGACGCCCCCGAGUACAAGCCCUGGGCCCUGGUGAUCCAGGACU |
| | | CCAACGGCGAGAACAAGAUCAAGAUGCUGUCCGGCGGCUCCAAGC |
| | | GGACCGCCGACGGCUCCGAGUUCGAGCCCAAGAAGAAGCGGAAGG |
| | | UGUGAUAGCUAGCACCAGCCUCAAGAACACCCGAAUGGAGUCUCU |
| | | AAGCUACAUAAUACCAACUUACACUUUACAAAAUGUUGUCCCCCA |
| | | AAAUGUAGCCAUUCGUAUCUGCUCCUAAUAAAAAGAAAGUUUCU |
| | | UCACAUUCUCUCGAGAAAAAAAAAAAAUGGAAAAAAAAAAAACG |
| | | GAAAAAAAAAAAAGGUAAAAAAAAAAAAUAUAAAAAAAAAAAAC |
| | | AUAAAAAAAAAAAACGAAAAAAAAAAAACGUAAAAAAAAAAAAC |
| | | UCAAAAAAAAAAAAGAUAAAAAAAAAAAACCUAAAAAAAAAAAA |
| | | UGUAAAAAAAAAAAAGGGAAAAAAAAAAAACGCAAAAAAAAAAA |
| | | ACACAAAAAAAAAAAAUGCAAAAAAAAAAAAUCGAAAAAAAAAA |
| | | AAUCUAAAAAAAAAAAACGAAAAAAAAAAAACCCAAAAAAAAAA |
| | | AAGACAAAAAAAAAAAAUAGAAAAAAAAAAAAGUUAAAAAAAAA |
| | | AAACUGAAAAAAAAAAAAUUUAAAAAAAAAAAAUCUAG |
| 20 | Open reading | AUGAAGCGGACCGCCGACGGCUCCGAGUUCGAGUCCCCCAAGAAG |
| | frame for | AAGCGGAAGGUGUCCUCCGAGACCGGCCCCGUGGCCGUGGACCCC |
| | BE4MAX protein | ACCCUGCGGCGGCGGAUCGAGCCCCACGAGUUCGAGGUGUUCUUC |
| | | GACCCCCGGGAGCUGCGGAAGGAGACCUGCCUGCUGUACGAGAUC |
| | | AACUGGGGCGGCCGGCACUCCAUCUGGCGGCACACCUCCCAGAAC |
| | | ACCAACAAGCACGUGGAGGUGAACUUCAUCGAGAAGUUCACCACC |
| | | GAGCGGUACUUCUGCCCCAACACCCGGUGCUCCAUCACCUGGUUC |
| | | CUGUCCUGGUCCCCCUGCGGCGAGUGCUCCCGGGCCAUCACCGAG |
| | | UUCCUGUCCCGGUACCCCCACGUGACCCUGUUCAUCUACAUCGCC |
| | | CGGCUGUACCACCACGCCGACCCCCGGAACCGGCAGGGCCUGCGG |
| | | GACCUGAUCUCCUCCGGCGUGACCAUCCAGAUCAUGACCGAGCAG |
| | | GAGUCCGGCUACUGCUGGCGGAACUUCGUGAACUACUCCCCCUCC |
| | | AACGAGGCCCACUGGCCCCGGUACCCCCACCUGUGGGUGCGGCUG |
| | | UACGUGCUGGAGCUGUACUGCAUCAUCCUGGGCCUGCCCCCCUGC |
| | | CUGAACAUCCUGCGGCGGAAGCAGCCCCAGCUGACCUUCUUCACC |
| | | AUCGCCCUGCAGUCCUGCCACUACCAGCGGCUGCCCCCCCACAUC |
| | | CUGUGGGCCACCGGCCUGAAGUCCGGCGGCUCCUCCGGCGGCUCC |
| | | UCCGGCUCCGAGACCCCCGGCACCUCCGAGUCCGCCACCCCCGAG |
| | | UCCUCCGGCGGCUCCUCCGGCGGCUCCGACAAGAAGUACUCCAUC |
| | | GGCCUGGCCAUCGGCACCAACUCCGUGGGCUGGGCCGUGAUCACC |
| | | GACGAGUACAAGGUGCCCUCCAAGAAGUUCAAGGUGCUGGGCAA |
| | | CACCGACCGGCACUCCAUCAAGAAGAACCUGAUCGGCGCCCUGCU |
| | | GUUCGACUCCGGCGAGACCGCCGAGGCCACCCGGCUGAAGCGGAC |
| | | CGCCCGGCGGCGGUACACCCGGCGGAAGAACCGGAUCUGCUACCU |
| | | GCAGGAGAUCUUCUCCAACGAGAUGGCCAAGGUGGACGACUCCU |
| | | UCUUCCACCGGCUGGAGGAGUCCUUCCUGGUGGAGGAGGACAAG |
| | | AAGCACGAGCGGCACCCCAUCUUCGGCAACAUCGUGGACGAGGUG |
| | | GCCUACCACGAGAAGUACCCCACCAUCUACCACCUGCGGAAGAAG |
| | | CUGGUGGACUCCACCGACAAGGCCGACCUGCGGCUGAUCUACCUG |
| | | GCCCUGGCCCACAUGAUCAAGUUCCGGGGCCACUUCCUGAUCGAG |
| | | GGCGACCUGAACCCCGACAACUCCGACGUGGACAAGCUGUUCAUC |
| | | CAGCUGGUGCAGACCUACAACCAGCUGUUCGAGGAGAACCCCAUC |
| | | AACGCCUCCGGCGUGGACGCCAAGGCCAUCCUGUCCGCCCGGCUG |
| | | UCCAAGUCCCGGCGGCUGGAGAACCUGAUCGCCCAGCUGCCCGGC |
| | | GAGAAGAAGAACGGCCUGUUCGGCAACCUGAUCGCCCUGUCCCUG |
| | | GGCCUGACCCCCAACUUCAAGUCCAACUUCGACCUGGCCGAGGAC |
| | | GCCAAGCUGCAGCUGUCCAAGGACACCUACGACGACGACCUGGAC |
| | | AACCUGCUGGCCCAGAUCGGCGACCAGUACGCCGACCUGUUCCUG |
| | | GCCGCCAAGAACCUGUCCGACGCCAUCCUGCUGUCCGACAUCCUG |
| | | CGGGUGAACACCGAGAUCACCAAGGCCCCCCUGUCCGCCUCCAUG |
| | | AUCAAGCGGUACGACGAGCACCACCAGGACCUGACCCUGCUGAAG |
| | | GCCCUGGUGCGGCAGCAGCUGCCCGAGAAGUACAAGGAGAUCUUC |
| | | UUCGACCAGUCCAAGAACGGCUACGCCGGCUACAUCGACGGCGGC |
| | | GCCUCCCAGGAGGAGUUCUACAAGUUCAUCAAGCCCAUCCUGGAG |
| | | AAGAUGGACGGCACCGAGGAGCUGCUGGUGAAGCUGAACCGGGA |
| | | GGACCUGCUGCGGAAGCAGCGGACCUUCGACAACGGCUCCAUCCC |
| | | CCACCAGAUCCACCUGGGCGAGCUGCACGCCAUCCUGCGGCGGCA |
| | | GGAGGACUUCUACCCCUUCCUGAAGGACAACCGGGAGAAGAUCG |
| | | AGAAGAUCCUGACCUUCCGGAUCCCCUACUACGUGGGCCCCCUGG |
| | | CCCGGGGCAACUCCCGGUUCGCCUGGAUGACCCGGAAGUCCGAGG |
| | | AGACCAUCACCCCCUGGAACUUCGAGGAGGUGGUGGACAAGGGC |
| | | GCCUCCGCCCAGUCCUUCAUCGAGCGGAUGACCAACUUCGACAAG |
| | | AACCUGCCCAACGAGAAGGUGCUGCCCAAGCACUCCCUGCUGUAC |
| | | GAGUACUUCACCGUGUACAACGAGCUGACCAAGGUGAAGUACGU |
| | | GACCGAGGGCAUGCGGAAGCCCGCCUUCCUGUCCGGCGAGCAGAA |
| | | GAAGGCCAUCGUGGACCUGCUGUUCAAGACCAACCGGAAGGUGA |
| | | CCGUGAAGCAGCUGAAGGAGGACUACUUCAAGAAGAUCGAGUGC |
| | | UUCGACUCCGUGGAGAUCUCCGGCGUGGAGGACCGGUUCAACGCC |
| | | UCCCUGGGCACCUACCACGACCUGCUGAAGAUCAUCAAGGACAAG |
| | | GACUUCCUGGACAACGAGGAGAACGAGGACAUCCUGGAGGACAU |
| | | CGUGCUGACCCUGACCCUGUUCGAGGACCGGGAGAUGAUCGAGG |
| | | AGCGGCUGAAGACCUACGCCCACCUGUUCGACGACAAGGUGAUGA |
| | | AGCAGCUGAAGCGGCGGCGGUACACCGGCUGGGGCCGGCUGUCCC |
| | | GGAAGCUGAUCAACGGCAUCCGGGACAAGCAGUCCGGCAAGACCA |
| | | UCCUGGACUUCCUGAAGUCCGACGGCUUCGCCAACCGGAACUUCA |
| | | UGCAGCUGAUCCACGACGACUCCCUGACCUUCAAGGAGGACAUCC |
| | | AGAAGGCCCAGGUGUCCGGCCAGGGCGACUCCCUGCACGAGCACA |
| | | UCGCCAACCUGGCCGGCUCCCCCGCCAUCAAGAAGGGCAUCCUGC |
| | | AGACCGUGAAGGUGGUGGACGAGCUGGUGAAGGUGAUGGGCCGG |
| | | CACAAGCCCGAGAACAUCGUGAUCGAGAUGGCCCGGGAGAACCAG |
| | | ACCACCCAGAAGGGCCAGAAGAACUCCCGGGAGCGGAUGAAGCGG |
| | | AUCGAGGAGGGCAUCAAGGAGCUGGGCUCCCAGAUCCUGAAGGA |
| | | GCACCCCGUGGAGAACACCCAGCUGCAGAACGAGAAGCUGUACCU |
| | | GUACUACCUGCAGAACGGCCGGGACAUGUACGUGGACCAGGAGC |
| | | UGGACAUCAACCGGCUGUCCGACUACGACGUGGACCACAUCGUGC |
| | | CCCAGUCCUUCCUGAAGGACGACUCCAUCGACAACAAGGUGCUGA |
| | | CCCGGUCCGACAAGAACCGGGGCAAGUCCGACAACGUGCCCUCCG |
| | | AGGAGGUGGUGAAGAAGAUGAAGAACUACUGGCGGCAGCUGCUG |
| | | AACGCCAAGCUGAUCACCCAGCGGAAGUUCGACAACCUGACCAAG |
| | | GCCGAGCGGGGCGGCCUGUCCGAGCUGGACAAGGCCGGCUUCAUC |
| | | AAGCGGCAGCUGGUGGAGACCCGGCAGAUCACCAAGCACGUGGCC |
| | | CAGAUCCUGGACUCCCGGAUGAACACCAAGUACGACGAGAACGAC |
| | | AAGCUGAUCCGGGAGGUGAAGGUGAUCACCCUGAAGUCCAAGCU |
| | | GGUGUCCGACUUCCGGAAGGACUUCCAGUUCUACAAGGUGCGGG |
| | | AGAUCAACAACUACCACCACGCCCACGACGCCUACCUGAACGCCG |
| | | UGGUGGGCACCGCCCUGAUCAAGAAGUACCCCAAGCUGGAGUCCG |
| | | AGUUCGUGUACGGCGACUACAAGGUGUACGACGUGCGGAAGAUG |
| | | AUCGCCAAGUCCGAGCAGGAGAUCGGCAAGGCCACCGCCAAGUAC |
| | | UUCUUCUACUCCAACAUCAUGAACUUCUUCAAGACCGAGAUCACC |
| | | CUGGCCAACGGCGAGAUCCGGAAGCGGCCCCUGAUCGAGACCAAC |
| | | GGCGAGACCGGCGAGAUCGUGUGGGACAAGGGCCGGGACUUCGC |
| | | CACCGUGCGGAAGGUGCUGUCCAUGCCCCAGGUGAACAUCGUGAA |
| | | GAAGACCGAGGUGCAGACCGGCGGCUUCUCCAAGGAGUCCAUCCU |
| | | GCCCAAGCGGAACUCCGACAAGCUGAUCGCCCGGAAGAAGGACUG |
| | | GGACCCCAAGAAGUACGGCGGCUUCGACUCCCCCACCGUGGCCUA |
| | | CUCCGUGCUGGUGGUGGCCAAGGUGGAGAAGGGCAAGUCCAAGA |
| | | AGCUGAAGUCCGUGAAGGAGCUGCUGGGCAUCACCAUCAUGGAG |
| | | CGGUCCUCCUUCGAGAAGAACCCCAUCGACUUCCUGGAGGCCAAG |
| | | GGCUACAAGGAGGUGAAGAAGGACCUGAUCAUCAAGCUGCCCAA |
| | | GUACUCCCUGUUCGAGCUGGAGAACGGCCGGAAGCGGAUGCUGG |
| | | CCUCCGCCGGCGAGCUGCAGAAGGGCAACGAGCUGGCCCUGCCCU |
| | | CCAAGUACGUGAACUUCCUGUACCUGGCCUCCCACUACGAGAAGC |
| | | UGAAGGGCUCCCCCGAGGACAACGAGCAGAAGCAGCUGUUCGUG |
| | | GAGCAGCACAAGCACUACCUGGACGAGAUCAUCGAGCAGAUCUCC |
| | | GAGUUCUCCAAGCGGGUGAUCCUGGCCGACGCCAACCUGGACAAG |
| | | GUGCUGUCCGCCUACAACAAGCACCGGGACAAGCCCAUCCGGGAG |
| | | CAGGCCGAGAACAUCAUCCACCUGUUCACCCUGACCAACCUGGGC |
| | | GCCCCCGCCGCCUUCAAGUACUUCGACACCACCAUCGACCGGAAG |
| | | CGGUACACCUCCACCAAGGAGGUGCUGGACGCCACCCUGAUCCAC |
| | | CAGUCCAUCACCGGCCUGUACGAGACCCGGAUCGACCUGUCCCAG |
| | | CUGGGCGGCGACUCCGGCGGCUCCGGCGGCUCCGGCGGCUCCACC |
| | | AACCUGUCCGACAUCAUCGAGAAGGAGACCGGCAAGCAGCUGGU |
| | | GAUCCAGGAGUCCAUCCUGAUGCUGCCCGAGGAGGUGGAGGAGG |
| | | UGAUCGGCAACAAGCCCGAGUCCGACAUCCUGGUGCACACCGCCU |
| | | ACGACGAGUCCACCGACGAGAACGUGAUGCUGCUGACCUCCGACG |
| | | CCCCCGAGUACAAGCCCUGGGCCCUGGUGAUCCAGGACUCCAACG |
| | | GCGAGAACAAGAUCAAGAUGCUGUCCGGCGGCUCCGGCGGCUCCG |
| | | GCGGCUCCACCAACCUGUCCGACAUCAUCGAGAAGGAGACCGGCA |
| | | AGCAGCUGGUGAUCCAGGAGUCCAUCCUGAUGCUGCCCGAGGAG |
| | | GUGGAGGAGGUGAUCGGCAACAAGCCCGAGUCCGACAUCCUGGU |
| | | GCACACCGCCUACGACGAGUCCACCGACGAGAACGUGAUGCUGCU |
| | | GACCUCCGACGCCCCCGAGUACAAGCCCUGGGCCCUGGUGAUCCA |
| | | GGACUCCAACGGCGAGAACAAGAUCAAGAUGCUGUCCGGCGGCUC |
| | | CAAGCGGACCGCCGACGGCUCCGAGUUCGAGCCCAAGAAGAAGCG |
| | | GAAGGUGUGAUAG |
| 21 | Amino acid | MKRTADGSEFESPKKKRKVSSETGPVAVDPTLRRRIEPHEFEVFFDPRE |
| | sequence for | LRKETCLLYEINWGGRHSIWRHTSQNTNKHVEVNFIEKFTTERYFCPNT |
| | BE4MAX protein | RCSITWFLSWSPCGECSRAITEFLSRYPHVTLFIYIARLYHHADPRNRQG |
| | | LRDLISSGVTIQIMTEQESGYCWRNFVNYSPSNEAHWPRYPHLWVRLY |
| | | VLELYCIILGLPPCLNILRRKQPQLTFFTIALQSCHYQRLPPHILWATGLK |
| | | SGGSSGGSSGSETPGTSESATPESSGGSSGGSDKKYSIGLAIGTNSVGW |
| | | AVITDEYKVPSKKFKVLGNTDRHSIKKNLIGALLFDSGETAEATRLKRT |
| | | ARRRYTRRKNRICYLQEIFSNEMAKVDDSFFHRLEESFLVEEDKKHER |
| | | HPIFGNIVDEVAYHEKYPTIYHLRKKLVDSTDKADLRLIYLALAHMIKF |
| | | RGHFLIEGDLNPDNSDVDKLFIQLVQTYNQLFEENPINASGVDAKAILS |
| | | ARLSKSRRLENLIAQLPGEKKNGLFGNLIALSLGLTPNFKSNFDLAEDA |
| | | KLQLSKDTYDDDLDNLLAQIGDQYADLFLAAKNLSDAILLSDILRVNT |
| | | EITKAPLSASMIKRYDEHHQDLTLLKALVRQQLPEKYKEIFFDQSKNGY |
| | | AGYIDGGASQEEFYKFIKPILEKMDGTEELLVKLNREDLLRKQRTFDN |
| | | GSIPHQIHLGELHAILRRQEDFYPFLKDNREKIEKILTFRIPYYVGPLARG |
| | | NSRFAWMTRKSEETITPWNFEEVVDKGASAQSFIERMTNFDKNLPNEK |
| | | VLPKHSLLYEYFTVYNELTKVKYVTEGMRKPAFLSGEQKKAIVDLLFK |
| | | TNRKVTVKQLKEDYFKKIECFDSVEISGVEDRFNASLGTYHDLLKIIKD |
| | | KDFLDNEENEDILEDIVLTLTLFEDREMIEERLKTYAHLFDDKVMKQL |
| | | KRRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDELV |
| | | KVMGRHKPENIVIEMARENQTTQKGQKNSRERMKRIEEGIKELGSQIL |
| | | KEHPVENTQLQNEKLYLYYLQNGRDMYVDQELDINRLSDYDVDHIVP |
| | | QSFLKDDSIDNKVLTRSDKNRGKSDNVPSEEVVKKMKNYWRQLLNA |
| | | KLITQRKFDNLTKAERGGLSELDKAGFIKRQLVETRQITKHVAQILDSR |
| | | MNTKYDENDKLIREVKVITLKSKLVSDFRKDFQFYKVREINNYHHAHD |
| | | AYLNAVVGTALIKKYPKLESEFVYGDYKVYDVRKMIAKSEQEIGKAT |
| | | AKYFFYSNIMNFFKTEITLANGEIRKRPLIETNGETGEIVWDKGRDFAT |
| | | VRKVLSMPQVNIVKKTEVQTGGFSKESILPKRNSDKLIARKKDWDPKK |
| | | YGGFDSPTVAYSVLVVAKVEKGKSKKLKSVKELLGITIMERSSFEKNPI |
| | | DFLEAKGYKEVKKDLIIKLPKYSLFELENGRKRMLASAGELQKGNELA |
| | | LPSKYVNFLYLASHYEKLKGSPEDNEQKQLFVEQHKHYLDEIIEQISEF |
| | | SKRVILADANLDKVLSAYNKHRDKPIREQAENIIHLFTLTNLGAPAAFK |
| | | YFDTTIDRKRYTSTKEVLDATLIHQSITGLYETRIDLSQLGGDSGGSGGS |
| | | GGSTNLSDIIEKETGKQLVIQESILMLPEEVEEVIGNKPESDILVHTAYDE |
| | | STDENVMLLTSDAPEYKPWALVIQDSNGENKIKMLSGGSGGSGGSTNL |
| | | SDIIEKETGKQLVIQESILMLPEEVEEVIGNKPESDILVHTAYDESTDENV |
| | | MLLTSDAPEYKPWALVIQDSNGENKIKMLSGGSKRTADGSEFEPKKKR |
| | | KV** |
| 22 | Amino acid | MEASPASGPRHLMDPHIFTSNFNNGIGRHKTYLCYEVERLDNGTSVKM |
| | sequence of H. | DQHRGFLHNQAKNLLCGFYGRHAELRFLDLVPSLQLDPAQIYRVTWFI |
| | sapiens | SWSPCFSWGCAGEVRAFLQENTHVRLRIFAARIYDYDPLYKEALQMLR |
| | APOBEC3A | DAGAQVSIMTYDEFKHCWDTFVDHQGCPFQPWDGLDEHSQALSGRL |
| | deaminase (A3A), | RAILQNQGN |
| | see BC22 | |
| 23 | | NOT USED |
| 24 | exemplary UGI | TNLSDIIEKETGKQLVIQESILMLPEEVEEVIGNKPESDILVHTAYDESTD |
| | | ENVMLLTSDAPEYKPWALVIQDSNGENKIKML |
| 25 | exemplary XTEN | SGSETPGTSESATPES |
| 26 | exemplary XTEN | SGSETPGTSESA |
| 27 | exemplary XTEN | SGSETPGTSESATPEGGSGGS |
| 28 | amino acid | GGGGSEAAAKEAAAK |
| | sequence for | |
| | exemplary linker | |
| 29 | amino acid | EAAAKGGGGSGGGGS |
| | sequence for | |
| | exemplary linker | |
| 30 | amino acid | EAAAKEAAAKEAAAK |
| | sequence for | |
| | exemplary linker | |
| 31 | amino acid | GGGGSGGGGSGGGGSGGGGS |
| | sequence for | |
| | exemplary linker | |
| 32 | amino acid | GGGGSGGGGSEAAAKEAAAK |
| | sequence for | |
| | exemplary linker | |
| 33 | amino acid | GGGGSEAAAKGGGGSGGGGS |
| | sequence for | |
| | exemplary linker | |
| 34 | amino acid | EAAAKEAAAKEAAAKGGGGSGGGGS |
| | sequence for | |
| | exemplary linker | |
| 35 | amino acid | EAAAKEAAAKEAAAKEAAAK |
| | sequence for | |
| | exemplary linker | |
| 36 | amino acid | GGGGSEAAAKEAAAKGGGGSEAAAK |
| | sequence for | |
| | exemplary linker | |
| 37 | amino acid | EAAAKEAAAKGGGGSGGGGSGGGGS |
| | sequence for | |
| | exemplary linker | |
| 38 | amino acid | EAAAKEAAAKGGGGSGGGGSEAAAK |
| | sequence for | |
| | exemplary linker | |
| 39 | amino acid | SGGS |
| | sequence for | |
| | exemplary linker | |
| | SGGS | |
| 40 | amino acid acid | PKKKRKV |
| | sequence for | |
| | SV40 NLS | |
| 41 | Amino acid | MDKKYSIGLAIGTNSVGWAVITDEYKVPSKKFKVLGNTDRHSIKKNLI |
| | sequence of Cas9 | GALLFDSGETAEATRLKRTARRRYTRRKNRICYLQEIFSNEMAKVDDS |
| | nickase (D10A) | FFHRLEESFLVEEDKKHERHPIFGNIVDEVAYHEKYPTIYHLRKKLVDS |
| | with 1x NLS as | TDKADLRLIYLALAHMIKFRGHFLIEGDLNPDNSDVDKLFIQLVQTYN |
| | thc C-terminal 7 | QLFEENPINASGVDAKAILSARLSKSRRLENLIAQLPGEKKNGLFGNLIA |
| | amino acids | LSLGLTPNFKSNFDLAEDAKLQLSKDTYDDDLDNLLAQIGDQYADLFL |
| | | AAKNLSDAILLSDILRVNTEITKAPLSASMIKRYDEHHQDLTLLKALVR |
| | | QQLPEKYKEIFFDQSKNGYAGYIDGGASQEEFYKFIKPILEKMDGTEEL |
| | | LVKLNREDLLRKQRTFDNGSIPHQIHLGELHAILRRQEDFYPFLKDNRE |
| | | KIEKILTFRIPYYVGPLARGNSRFAWMTRKSEETITPWNFEEVVDKGAS |
| | | AQSFIERMTNFDKNLPNEKVLPKHSLLYEYFTVYNELTKVKYVTEGMR |
| | | KPAFLSGEQKKAIVDLLFKTNRKVTVKQLKEDYFKKIECFDSVEISGVE |
| | | DRFNASLGTYHDLLKIIKDKDFLDNEENEDILEDIVLTLTLFEDREMIEE |
| | | RLKTYAHLFDDKVMKQLKRRRYTGWGRLSRKLINGIRDKQSGKTILD |
| | | FLKSDGFANRNFMQLIHDDSLTFKEDIQKAQVSGQGDSLHEHIANLAG |
| | | SPAIKKGILQTVKVVDELVKVMGRHKPENIVIEMARENQTTQKGQKNS |
| | | RERMKRIEEGIKELGSQILKEHPVENTQLQNEKLYLYYLQNGRDMYVD |
| | | QELDINRLSDYDVDHIVPQSFLKDDSIDNKVLTRSDKNRGKSDNVPSEE |
| | | VVKKMKNYWRQLLNAKLITQRKFDNLTKAERGGLSELDKAGFIKRQL |
| | | VETRQITKHVAQILDSRMNTKYDENDKLIREVKVITLKSKLVSDFRKDF |
| | | QFYKVREINNYHHAHDAYLNAVVGTALIKKYPKLESEFVYGDYKVYD |
| | | VRKMIAKSEQEIGKATAKYFFYSNIMNFFKTEITLANGEIRKRPLIETNG |
| | | ETGEIVWDKGRDFATVRKVLSMPQVNIVKKTEVQTGGFSKESILPKRN |
| | | SDKLIARKKDWDPKKYGGFDSPTVAYSVLVVAKVEKGKSKKLKSVKE |
| | | LLGITIMERSSFEKNPIDFLEAKGYKEVKKDLIIKLPKYSLFELENGRKR |
| | | MLASAGELQKGNELALPSKYVNFLYLASHYEKLKGSPEDNEQKQLFV |
| | | EQHKHYLDEIIEQISEFSKRVILADANLDKVLSAYNKHRDKPIREQAENI |
| | | IHLFTLTNLGAPAAFKYFDTTIDRKRYTSTKEVLDATLIHQSITGLYETR |
| | | IDLSQLGGDGGGSPKKKRKV |
| 42 | Cas9 nickase | GACAAGAAGUACAGCAUCGGACUGGCAAUCGGAACAAACAGCGU |
| | (D10A) mRNA | CGGAUGGGCAGUCAUCACAGACGAAUACAAGGUCCCGAGCAAGA |
| | coding sequence | AGUUCAAGGUCCUGGGAAACACAGACAGACACAGCAUCAAGAAG |
| | using minimal | AACCUGAUCGGAGCACUGCUGUUCGACAGCGGAGAAACAGCAGA |
| | uridine codons as | AGCAACAAGACUGAAGAGAACAGCAAGAAGAAGAUACACAAGAA |
| | listed in Table 3 | GAAAGAACAGAAUCUGCUACCUGCAGGAAAUCUUCAGCAACGAA |
| | (no start or stop | AUGGCAAAGGUCGACGACAGCUUCUUCCACAGACUGGAAGAAAG |
| | codons; suitable | CUUCCUGGUCGAAGAAGACAAGAAGCACGAAAGACACCCGAUCU |
| | for inclusion in | UCGGAAACAUCGUCGACGAAGUCGCAUACCACGAAAAGUACCCGA |
| | fusion protein | CAAUCUACCACCUGAGAAAGAAGCUGGUCGACAGCACAGACAAG |
| | coding sequence) | GCAGACCUGAGACUGAUCUACCUGGCACUGGCACACAUGAUCAAG |
| | | UUCAGAGGACACUUCCUGAUCGAAGGAGACCUGAACCCGGACAAC |
| | | AGCGACGUCGACAAGCUGUUCAUCCAGCUGGUCCAGACAUACAAC |
| | | CAGCUGUUCGAAGAAAACCCGAUCAACGCAAGCGGAGUCGACGCA |
| | | AAGGCAAUCCUGAGCGCAAGACUGAGCAAGAGCAGAAGACUGGA |
| | | AAACCUGAUCGCACAGCUGCCGGGAGAAAAGAAGAACGGACUGU |
| | | UCGGAAACCUGAUCGCACUGAGCCUGGGACUGACACCGAACUUCA |
| | | AGAGCAACUUCGACCUGGCAGAAGACGCAAAGCUGCAGCUGAGC |
| | | AAGGACACAUACGACGACGACCUGGACAACCUGCUGGCACAGAUC |
| | | GGAGACCAGUACGCAGACCUGUUCCUGGCAGCAAAGAACCUGAGC |
| | | GACGCAAUCCUGCUGAGCGACAUCCUGAGAGUCAACACAGAAAUC |
| | | ACAAAGGCACCGCUGAGCGCAAGCAUGAUCAAGAGAUACGACGA |
| | | ACACCACCAGGACCUGACACUGCUGAAGGCACUGGUCAGACAGCA |
| | | GCUGCCGGAAAAGUACAAGGAAAUCUUCUUCGACCAGAGCAAGA |
| | | ACGGAUACGCAGGAUACAUCGACGGAGGAGCAAGCCAGGAAGAA |
| | | UUCUACAAGUUCAUCAAGCCGAUCCUGGAAAAGAUGGACGGAAC |
| | | AGAAGAACUGCUGGUCAAGCUGAACAGAGAAGACCUGCUGAGAA |
| | | AGCAGAGAACAUUCGACAACGGAAGCAUCCCGCACCAGAUCCACC |
| | | UGGGAGAACUGCACGCAAUCCUGAGAAGACAGGAAGACUUCUAC |
| | | CCGUUCCUGAAGGACAACAGAGAAAAGAUCGAAAAGAUCCUGAC |
| | | AUUCAGAAUCCCGUACUACGUCGGACCGCUGGCAAGAGGAAACA |
| | | GCAGAUUCGCAUGGAUGACAAGAAAGAGCGAAGAAACAAUCACA |
| | | CCGUGGAACUUCGAAGAAGUCGUCGACAAGGGAGCAAGCGCACA |
| | | GAGCUUCAUCGAAAGAAUGACAAACUUCGACAAGAACCUGCCGA |
| | | ACGAAAAGGUCCUGCCGAAGCACAGCCUGCUGUACGAAUACUUCA |
| | | CAGUCUACAACGAACUGACAAAGGUCAAGUACGUCACAGAAGGA |
| | | AUGAGAAAGCCGGCAUUCCUGAGCGGAGAACAGAAGAAGGCAAU |
| | | CGUCGACCUGCUGUUCAAGACAAACAGAAAGGUCACAGUCAAGC |
| | | AGCUGAAGGAAGACUACUUCAAGAAGAUCGAAUGCUUCGACAGC |
| | | GUCGAAAUCAGCGGAGUCGAAGACAGAUUCAACGCAAGCCUGGG |
| | | AACAUACCACGACCUGCUGAAGAUCAUCAAGGACAAGGACUUCCU |
| | | GGACAACGAAGAAAACGAAGACAUCCUGGAAGACAUCGUCCUGA |
| | | CACUGACACUGUUCGAAGACAGAGAAAUGAUCGAAGAAAGACUG |
| | | AAGACAUACGCACACCUGUUCGACGACAAGGUCAUGAAGCAGCU |
| | | GAAGAGAAGAAGAUACACAGGAUGGGGAAGACUGAGCAGAAAGC |
| | | UGAUCAACGGAAUCAGAGACAAGCAGAGCGGAAAGACAAUCCUG |
| | | GACUUCCUGAAGAGCGACGGAUUCGCAAACAGAAACUUCAUGCA |
| | | GCUGAUCCACGACGACAGCCUGACAUUCAAGGAAGACAUCCAGAA |
| | | GGCACAGGUCAGCGGACAGGGAGACAGCCUGCACGAACACAUCGC |
| | | AAACCUGGCAGGAAGCCCGGCAAUCAAGAAGGGAAUCCUGCAGA |
| | | CAGUCAAGGUCGUCGACGAACUGGUCAAGGUCAUGGGAAGACAC |
| | | AAGCCGGAAAACAUCGUCAUCGAAAUGGCAAGAGAAAACCAGAC |
| | | AACACAGAAGGGACAGAAGAACAGCAGAGAAAGAAUGAAGAGAA |
| | | UCGAAGAAGGAAUCAAGGAACUGGGAAGCCAGAUCCUGAAGGAA |
| | | CACCCGGUCGAAAACACACAGCUGCAGAACGAAAAGCUGUACCUG |
| | | UACUACCUGCAGAACGGAAGAGACAUGUACGUCGACCAGGAACU |
| | | GGACAUCAACAGACUGAGCGACUACGACGUCGACCACAUCGUCCC |
| | | GCAGAGCUUCCUGAAGGACGACAGCAUCGACAACAAGGUCCUGAC |
| | | AAGAAGCGACAAGAACAGAGGAAAGAGCGACAACGUCCCGAGCG |
| | | AAGAAGUCGUCAAGAAGAUGAAGAACUACUGGAGACAGCUGCUG |
| | | AACGCAAAGCUGAUCACACAGAGAAAGUUCGACAACCUGACAAA |
| | | GGCAGAGAGAGGAGGACUGAGCGAACUGGACAAGGCAGGAUUCA |
| | | UCAAGAGACAGCUGGUCGAAACAAGACAGAUCACAAAGCACGUC |
| | | GCACAGAUCCUGGACAGCAGAAUGAACACAAAGUACGACGAAAA |
| | | CGACAAGCUGAUCAGAGAAGUCAAGGUCAUCACACUGAAGAGCA |
| | | AGCUGGUCAGCGACUUCAGAAAGGACUUCCAGUUCUACAAGGUC |
| | | AGAGAAAUCAACAACUACCACCACGCACACGACGCAUACCUGAAC |
| | | GCAGUCGUCGGAACAGCACUGAUCAAGAAGUACCCGAAGCUGGA |
| | | AAGCGAAUUCGUCUACGGAGACUACAAGGUCUACGACGUCAGAA |
| | | AGAUGAUCGCAAAGAGCGAACAGGAAAUCGGAAAGGCAACAGCA |
| | | AAGUACUUCUUCUACAGCAACAUCAUGAACUUCUUCAAGACAGA |
| | | AAUCACACUGGCAAACGGAGAAAUCAGAAAGAGACCGCUGAUCG |
| | | AAACAAACGGAGAAACAGGAGAAAUCGUCUGGGACAAGGGAAGA |
| | | GACUUCGCAACAGUCAGAAAGGUCCUGAGCAUGCCGCAGGUCAAC |
| | | AUCGUCAAGAAGACAGAAGUCCAGACAGGAGGAUUCAGCAAGGA |
| | | AAGCAUCCUGCCGAAGAGAAACAGCGACAAGCUGAUCGCAAGAA |
| | | AGAAGGACUGGGACCCGAAGAAGUACGGAGGAUUCGACAGCCCG |
| | | ACAGUCGCAUACAGCGUCCUGGUCGUCGCAAAGGUCGAAAAGGG |
| | | AAAGAGCAAGAAGCUGAAGAGCGUCAAGGAACUGCUGGGAAUCA |
| | | CAAUCAUGGAAAGAAGCAGCUUCGAAAAGAACCCGAUCGACUUC |
| | | CUGGAAGCAAAGGGAUACAAGGAAGUCAAGAAGGACCUGAUCAU |
| | | CAAGCUGCCGAAGUACAGCCUGUUCGAACUGGAAAACGGAAGAA |
| | | AGAGAAUGCUGGCAAGCGCAGGAGAACUGCAGAAGGGAAACGAA |
| | | CUGGCACUGCCGAGCAAGUACGUCAACUUCCUGUACCUGGCAAGC |
| | | CACUACGAAAAGCUGAAGGGAAGCCCGGAAGACAACGAACAGAA |
| | | GCAGCUGUUCGUCGAACAGCACAAGCACUACCUGGACGAAAUCAU |
| | | CGAACAGAUCAGCGAAUUCAGCAAGAGAGUCAUCCUGGCAGACG |
| | | CAAACCUGGACAAGGUCCUGAGCGCAUACAACAAGCACAGAGACA |
| | | AGCCGAUCAGAGAACAGGCAGAAAACAUCAUCCACCUGUUCACAC |
| | | UGACAAACCUGGGAGCACCGGCAGCAUUCAAGUACUUCGACACAA |
| | | CAAUCGACAGAAAGAGAUACACAAGCACAAAGGAAGUCCUGGAC |
| | | GCAACACUGAUCCACCAGAGCAUCACAGGACUGUACGAAACAAGA |
| | | AUCGACCUGAGCCAGCUGGGAGGAGACGGAGGAGGAAGCCCGAA |
| | | GAAGAAGAGAAAGGUC |
| 43 | Amino acid | MDKKYSIGLAIGTNSVGWAVITDEYKVPSKKFKVLGNTDRHSIKKNLI |
| | sequence of Cas9 | GALLFDSGETAEATRLKRTARRRYTRRKNRICYLQEIFSNEMAKVDDS |
| | nickase (without | FFHRLEESFLVEEDKKHERHPIFGNIVDEVAYHEKYPTIYHLRKKLVDS |
| | NLS) | TDKADLRLIYLALAHMIKFRGHFLIEGDLNPDNSDVDKLFIQLVQTYN |
| | | QLFEENPINASGVDAKAILSARLSKSRRLENLIAQLPGEKKNGLFGNLIA |
| | | LSLGLTPNFKSNFDLAEDAKLQLSKDTYDDDLDNLLAQIGDQYADLFL |
| | | AAKNLSDAILLSDILRVNTEITKAPLSASMIKRYDEHHQDLTLLKALVR |
| | | QQLPEKYKEIFFDQSKNGYAGYIDGGASQEEFYKFIKPILEKMDGTEEL |
| | | LVKLNREDLLRKQRTFDNGSIPHQIHLGELHAILRRQEDFYPFLKDNRE |
| | | KIEKILTFRIPYYVGPLARGNSRFAWMTRKSEETITPWNFEEVVDKGAS |
| | | AQSFIERMTNFDKNLPNEKVLPKHSLLYEYFTVYNELTKVKYVTEGMR |
| | | KPAFLSGEQKKAIVDLLFKTNRKVTVKQLKEDYFKKIECFDSVEISGVE |
| | | DRFNASLGTYHDLLKIIKDKDFLDNEENEDILEDIVLTLTLFEDREMIEE |
| | | RLKTYAHLFDDKVMKQLKRRRYTGWGRLSRKLINGIRDKQSGKTILD |
| | | FLKSDGFANRNFMQLIHDDSLTFKEDIQKAQVSGQGDSLHEHIANLAG |
| | | SPAIKKGILQTVKVVDELVKVMGRHKPENIVIEMARENQTTQKGQKNS |
| | | RERMKRIEEGIKELGSQILKEHPVENTQLQNEKLYLYYLQNGRDMYVD |
| | | QELDINRLSDYDVDHIVPQSFLKDDSIDNKVLTRSDKNRGKSDNVPSEE |
| | | VVKKMKNYWRQLLNAKLITQRKFDNLTKAERGGLSELDKAGFIKRQL |
| | | VETRQITKHVAQILDSRMNTKYDENDKLIREVKVITLKSKLVSDFRKDF |
| | | QFYKVREINNYHHAHDAYLNAVVGTALIKKYPKLESEFVYGDYKVYD |
| | | VRKMIAKSEQEIGKATAKYFFYSNIMNFFKTEITLANGEIRKRPLIETNG |
| | | ETGEIVWDKGRDFATVRKVLSMPQVNIVKKTEVQTGGFSKESILPKRN |
| | | SDKLIARKKDWDPKKYGGFDSPTVAYSVLVVAKVEKGKSKKLKSVKE |
| | | LLGITIMERSSFEKNPIDFLEAKGYKEVKKDLIIKLPKYSLFELENGRKR |
| | | MLASAGELQKGNELALPSKYVNFLYLASHYEKLKGSPEDNEQKQLFV |
| | | EQHKHYLDEIIEQISEFSKRVILADANLDKVLSAYNKHRDKPIREQAENI |
| | | IHLFTLTNLGAPAAFKYFDTTIDRKRYTSTKEVLDATLIHQSITGLYETR |
| | | IDLSQLGGD |
| 44 | Cas9 nickase | GACAAGAAGUACAGCAUCGGACUGGCAAUCGGAACAAACAGCGU |
| | coding sequence | CGGAUGGGCAGUCAUCACAGACGAAUACAAGGUCCCGAGCAAGA |
| | encoding SEQ ID | AGUUCAAGGUCCUGGGAAACACAGACAGACACAGCAUCAAGAAG |
| | NO: 43 using | AACCUGAUCGGAGCACUGCUGUUCGACAGCGGAGAAACAGCAGA |
| | minimal uridine | AGCAACAAGACUGAAGAGAACAGCAAGAAGAAGAUACACAAGAA |
| | codons as listed | GAAAGAACAGAAUCUGCUACCUGCAGGAAAUCUUCAGCAACGAA |
| | in Table 3 (no | AUGGCAAAGGUCGACGACAGCUUCUUCCACAGACUGGAAGAAAG |
| | start or stop | CUUCCUGGUCGAAGAAGACAAGAAGCACGAAAGACACCCGAUCU |
| | codons; suitable | UCGGAAACAUCGUCGACGAAGUCGCAUACCACGAAAAGUACCCGA |
| | for inclusion in | CAAUCUACCACCUGAGAAAGAAGCUGGUCGACAGCACAGACAAG |
| | fusion protein | GCAGACCUGAGACUGAUCUACCUGGCACUGGCACACAUGAUCAAG |
| | coding sequence) | UUCAGAGGACACUUCCUGAUCGAAGGAGACCUGAACCCGGACAAC |
| | | AGCGACGUCGACAAGCUGUUCAUCCAGCUGGUCCAGACAUACAAC |
| | | CAGCUGUUCGAAGAAAACCCGAUCAACGCAAGCGGAGUCGACGCA |
| | | AAGGCAAUCCUGAGCGCAAGACUGAGCAAGAGCAGAAGACUGGA |
| | | AAACCUGAUCGCACAGCUGCCGGGAGAAAAGAAGAACGGACUGU |
| | | UCGGAAACCUGAUCGCACUGAGCCUGGGACUGACACCGAACUUCA |
| | | AGAGCAACUUCGACCUGGCAGAAGACGCAAAGCUGCAGCUGAGC |
| | | AAGGACACAUACGACGACGACCUGGACAACCUGCUGGCACAGAUC |
| | | GGAGACCAGUACGCAGACCUGUUCCUGGCAGCAAAGAACCUGAGC |
| | | GACGCAAUCCUGCUGAGCGACAUCCUGAGAGUCAACACAGAAAUC |
| | | ACAAAGGCACCGCUGAGCGCAAGCAUGAUCAAGAGAUACGACGA |
| | | ACACCACCAGGACCUGACACUGCUGAAGGCACUGGUCAGACAGCA |
| | | GCUGCCGGAAAAGUACAAGGAAAUCUUCUUCGACCAGAGCAAGA |
| | | ACGGAUACGCAGGAUACAUCGACGGAGGAGCAAGCCAGGAAGAA |
| | | UUCUACAAGUUCAUCAAGCCGAUCCUGGAAAAGAUGGACGGAAC |
| | | AGAAGAACUGCUGGUCAAGCUGAACAGAGAAGACCUGCUGAGAA |
| | | AGCAGAGAACAUUCGACAACGGAAGCAUCCCGCACCAGAUCCACC |
| | | UGGGAGAACUGCACGCAAUCCUGAGAAGACAGGAAGACUUCUAC |
| | | CCGUUCCUGAAGGACAACAGAGAAAAGAUCGAAAAGAUCCUGAC |
| | | AUUCAGAAUCCCGUACUACGUCGGACCGCUGGCAAGAGGAAACA |
| | | GCAGAUUCGCAUGGAUGACAAGAAAGAGCGAAGAAACAAUCACA |
| | | CCGUGGAACUUCGAAGAAGUCGUCGACAAGGGAGCAAGCGCACA |
| | | GAGCUUCAUCGAAAGAAUGACAAACUUCGACAAGAACCUGCCGA |
| | | ACGAAAAGGUCCUGCCGAAGCACAGCCUGCUGUACGAAUACUUCA |
| | | CAGUCUACAACGAACUGACAAAGGUCAAGUACGUCACAGAAGGA |
| | | AUGAGAAAGCCGGCAUUCCUGAGCGGAGAACAGAAGAAGGCAAU |
| | | CGUCGACCUGCUGUUCAAGACAAACAGAAAGGUCACAGUCAAGC |
| | | AGCUGAAGGAAGACUACUUCAAGAAGAUCGAAUGCUUCGACAGC |
| | | GUCGAAAUCAGCGGAGUCGAAGACAGAUUCAACGCAAGCCUGGG |
| | | AACAUACCACGACCUGCUGAAGAUCAUCAAGGACAAGGACUUCCU |
| | | GGACAACGAAGAAAACGAAGACAUCCUGGAAGACAUCGUCCUGA |
| | | CACUGACACUGUUCGAAGACAGAGAAAUGAUCGAAGAAAGACUG |
| | | AAGACAUACGCACACCUGUUCGACGACAAGGUCAUGAAGCAGCU |
| | | GAAGAGAAGAAGAUACACAGGAUGGGGAAGACUGAGCAGAAAGC |
| | | UGAUCAACGGAAUCAGAGACAAGCAGAGCGGAAAGACAAUCCUG |
| | | GACUUCCUGAAGAGCGACGGAUUCGCAAACAGAAACUUCAUGCA |
| | | GCUGAUCCACGACGACAGCCUGACAUUCAAGGAAGACAUCCAGAA |
| | | GGCACAGGUCAGCGGACAGGGAGACAGCCUGCACGAACACAUCGC |
| | | AAACCUGGCAGGAAGCCCGGCAAUCAAGAAGGGAAUCCUGCAGA |
| | | CAGUCAAGGUCGUCGACGAACUGGUCAAGGUCAUGGGAAGACAC |
| | | AAGCCGGAAAACAUCGUCAUCGAAAUGGCAAGAGAAAACCAGAC |
| | | AACACAGAAGGGACAGAAGAACAGCAGAGAAAGAAUGAAGAGAA |
| | | UCGAAGAAGGAAUCAAGGAACUGGGAAGCCAGAUCCUGAAGGAA |
| | | CACCCGGUCGAAAACACACAGCUGCAGAACGAAAAGCUGUACCUG |
| | | UACUACCUGCAGAACGGAAGAGACAUGUACGUCGACCAGGAACU |
| | | GGACAUCAACAGACUGAGCGACUACGACGUCGACCACAUCGUCCC |
| | | GCAGAGCUUCCUGAAGGACGACAGCAUCGACAACAAGGUCCUGAC |
| | | AAGAAGCGACAAGAACAGAGGAAAGAGCGACAACGUCCCGAGCG |
| | | AAGAAGUCGUCAAGAAGAUGAAGAACUACUGGAGACAGCUGCUG |
| | | AACGCAAAGCUGAUCACACAGAGAAAGUUCGACAACCUGACAAA |
| | | GGCAGAGAGAGGAGGACUGAGCGAACUGGACAAGGCAGGAUUCA |
| | | UCAAGAGACAGCUGGUCGAAACAAGACAGAUCACAAAGCACGUC |
| | | GCACAGAUCCUGGACAGCAGAAUGAACACAAAGUACGACGAAAA |
| | | CGACAAGCUGAUCAGAGAAGUCAAGGUCAUCACACUGAAGAGCA |
| | | AGCUGGUCAGCGACUUCAGAAAGGACUUCCAGUUCUACAAGGUC |
| | | AGAGAAAUCAACAACUACCACCACGCACACGACGCAUACCUGAAC |
| | | GCAGUCGUCGGAACAGCACUGAUCAAGAAGUACCCGAAGCUGGA |
| | | AAGCGAAUUCGUCUACGGAGACUACAAGGUCUACGACGUCAGAA |
| | | AGAUGAUCGCAAAGAGCGAACAGGAAAUCGGAAAGGCAACAGCA |
| | | AAGUACUUCUUCUACAGCAACAUCAUGAACUUCUUCAAGACAGA |
| | | AAUCACACUGGCAAACGGAGAAAUCAGAAAGAGACCGCUGAUCG |
| | | AAACAAACGGAGAAACAGGAGAAAUCGUCUGGGACAAGGGAAGA |
| | | GACUUCGCAACAGUCAGAAAGGUCCUGAGCAUGCCGCAGGUCAAC |
| | | AUCGUCAAGAAGACAGAAGUCCAGACAGGAGGAUUCAGCAAGGA |
| | | AAGCAUCCUGCCGAAGAGAAACAGCGACAAGCUGAUCGCAAGAA |
| | | AGAAGGACUGGGACCCGAAGAAGUACGGAGGAUUCGACAGCCCG |
| | | ACAGUCGCAUACAGCGUCCUGGUCGUCGCAAAGGUCGAAAAGGG |
| | | AAAGAGCAAGAAGCUGAAGAGCGUCAAGGAACUGCUGGGAAUCA |
| | | CAAUCAUGGAAAGAAGCAGCUUCGAAAAGAACCCGAUCGACUUC |
| | | CUGGAAGCAAAGGGAUACAAGGAAGUCAAGAAGGACCUGAUCAU |
| | | CAAGCUGCCGAAGUACAGCCUGUUCGAACUGGAAAACGGAAGAA |
| | | AGAGAAUGCUGGCAAGCGCAGGAGAACUGCAGAAGGGAAACGAA |
| | | CUGGCACUGCCGAGCAAGUACGUCAACUUCCUGUACCUGGCAAGC |
| | | CACUACGAAAAGCUGAAGGGAAGCCCGGAAGACAACGAACAGAA |
| | | GCAGCUGUUCGUCGAACAGCACAAGCACUACCUGGACGAAAUCAU |
| | | CGAACAGAUCAGCGAAUUCAGCAAGAGAGUCAUCCUGGCAGACG |
| | | CAAACCUGGACAAGGUCCUGAGCGCAUACAACAAGCACAGAGACA |
| | | AGCCGAUCAGAGAACAGGCAGAAAACAUCAUCCACCUGUUCACAC |
| | | UGACAAACCUGGGAGCACCGGCAGCAUUCAAGUACUUCGACACAA |
| | | CAAUCGACAGAAAGAGAUACACAAGCACAAAGGAAGUCCUGGAC |
| | | GCAACACUGAUCCACCAGAGCAUCACAGGACUGUACGAAACAAGA |
| | | AUCGACCUGAGCCAGCUGGGAGGAGAC |
| 45 | Amino acid | DKKYSIGLAIGTNSVGWAVITDEYKVPSKKFKVLGNTDRHSIKKNLIG |
| | sequence of Cas9 | ALLFDSGETAEATRLKRTARRRYTRRKNRICYLQEIFSNEMAKVDDSFF |
| | nickase with two | HRLEESFLVEEDKKHERHPIFGNIVDEVAYHEKYPTIYHLRKKLVDSTD |
| | nuclear | KADLRLIYLALAHMIKFRGHFLIEGDLNPDNSDVDKLFIQLVQTYNQLF |
| | localization | EENPINASGVDAKAILSARLSKSRRLENLIAQLPGEKKNGLFGNLIALSL |
| | signals as thc C- | GLTPNFKSNFDLAEDAKLQLSKDTYDDDLDNLLAQIGDQYADLFLAA |
| | terminal amino | KNLSDAILLSDILRVNTEITKAPLSASMIKRYDEHHQDLTLLKALVRQQ |
| | acids | LPEKYKEIFFDQSKNGYAGYIDGGASQEEFYKFIKPILEKMDGTEELLV |
| | | KLNREDLLRKQRTFDNGSIPHQIHLGELHAILRRQEDFYPFLKDNREKIE |
| | | KILTFRIPYYVGPLARGNSRFAWMTRKSEETITPWNFEEVVDKGASAQ |
| | | SFIERMTNFDKNLPNEKVLPKHSLLYEYFTVYNELTKVKYVTEGMRKP |
| | | AFLSGEQKKAIVDLLFKTNRKVTVKQLKEDYFKKIECFDSVEISGVEDR |
| | | FNASLGTYHDLLKIIKDKDFLDNEENEDILEDIVLTLTLFEDREMIEERL |
| | | KTYAHLFDDKVMKQLKRRRYTGWGRLSRKLINGIRDKQSGKTILDFL |
| | | KSDGFANRNFMQLIHDDSLTFKEDIQKAQVSGQGDSLHEHIANLAGSP |
| | | AIKKGILQTVKVVDELVKVMGRHKPENIVIEMARENQTTQKGQKNSR |
| | | ERMKRIEEGIKELGSQILKEHPVENTQLQNEKLYLYYLQNGRDMYVDQ |
| | | ELDINRLSDYDVDHIVPQSFLKDDSIDNKVLTRSDKNRGKSDNVPSEEV |
| | | VKKMKNYWRQLLNAKLITQRKFDNLTKAERGGLSELDKAGFIKRQLV |
| | | ETRQITKHVAQILDSRMNTKYDENDKLIREVKVITLKSKLVSDFRKDFQ |
| | | FYKVREINNYHHAHDAYLNAVVGTALIKKYPKLESEFVYGDYKVYDV |
| | | RKMIAKSEQEIGKATAKYFFYSNIMNFFKTEITLANGEIRKRPLIETNGE |
| | | TGEIVWDKGRDFATVRKVLSMPQVNIVKKTEVQTGGFSKESILPKRNS |
| | | DKLIARKKDWDPKKYGGFDSPTVAYSVLVVAKVEKGKSKKLKSVKEL |
| | | LGITIMERSSFEKNPIDFLEAKGYKEVKKDLIIKLPKYSLFELENGRKRM |
| | | LASAGELQKGNELALPSKYVNFLYLASHYEKLKGSPEDNEQKQLFVEQ |
| | | HKHYLDEIIEQISEFSKRVILADANLDKVLSAYNKHRDKPIREQAENIIH |
| | | LFTLTNLGAPAAFKYFDTTIDRKRYTSTKEVLDATLIHQSITGLYETRID |
| | | LSQLGGDGSGSPKKKRKVDGSPKKKRKVDSG |
| 46 | Cas9 nickase | GACAAGAAGUACAGCAUCGGACUGGCAAUCGGAACAAACAGCGU |
| | coding sequence | CGGAUGGGCAGUCAUCACAGACGAAUACAAGGUCCCGAGCAAGA |
| | encoding SEQ ID | AGUUCAAGGUCCUGGGAAACACAGACAGACACAGCAUCAAGAAG |
| | NO: 45 using | AACCUGAUCGGAGCACUGCUGUUCGACAGCGGAGAAACAGCAGA |
| | minimal uridine | AGCAACAAGACUGAAGAGAACAGCAAGAAGAAGAUACACAAGAA |
| | codons as listed | GAAAGAACAGAAUCUGCUACCUGCAGGAAAUCUUCAGCAACGAA |
| | in Table 3 (no | AUGGCAAAGGUCGACGACAGCUUCUUCCACAGACUGGAAGAAAG |
| | start or stop | CUUCCUGGUCGAAGAAGACAAGAAGCACGAAAGACACCCGAUCU |
| | codons; suitable | UCGGAAACAUCGUCGACGAAGUCGCAUACCACGAAAAGUACCCGA |
| | for inclusion in | CAAUCUACCACCUGAGAAAGAAGCUGGUCGACAGCACAGACAAG |
| | fusion protein | GCAGACCUGAGACUGAUCUACCUGGCACUGGCACACAUGAUCAAG |
| | coding sequence) | UUCAGAGGACACUUCCUGAUCGAAGGAGACCUGAACCCGGACAAC |
| | | AGCGACGUCGACAAGCUGUUCAUCCAGCUGGUCCAGACAUACAAC |
| | | CAGCUGUUCGAAGAAAACCCGAUCAACGCAAGCGGAGUCGACGCA |
| | | AAGGCAAUCCUGAGCGCAAGACUGAGCAAGAGCAGAAGACUGGA |
| | | AAACCUGAUCGCACAGCUGCCGGGAGAAAAGAAGAACGGACUGU |
| | | UCGGAAACCUGAUCGCACUGAGCCUGGGACUGACACCGAACUUCA |
| | | AGAGCAACUUCGACCUGGCAGAAGACGCAAAGCUGCAGCUGAGC |
| | | AAGGACACAUACGACGACGACCUGGACAACCUGCUGGCACAGAUC |
| | | GGAGACCAGUACGCAGACCUGUUCCUGGCAGCAAAGAACCUGAGC |
| | | GACGCAAUCCUGCUGAGCGACAUCCUGAGAGUCAACACAGAAAUC |
| | | ACAAAGGCACCGCUGAGCGCAAGCAUGAUCAAGAGAUACGACGA |
| | | ACACCACCAGGACCUGACACUGCUGAAGGCACUGGUCAGACAGCA |
| | | GCUGCCGGAAAAGUACAAGGAAAUCUUCUUCGACCAGAGCAAGA |
| | | ACGGAUACGCAGGAUACAUCGACGGAGGAGCAAGCCAGGAAGAA |
| | | UUCUACAAGUUCAUCAAGCCGAUCCUGGAAAAGAUGGACGGAAC |
| | | AGAAGAACUGCUGGUCAAGCUGAACAGAGAAGACCUGCUGAGAA |
| | | AGCAGAGAACAUUCGACAACGGAAGCAUCCCGCACCAGAUCCACC |
| | | UGGGAGAACUGCACGCAAUCCUGAGAAGACAGGAAGACUUCUAC |
| | | CCGUUCCUGAAGGACAACAGAGAAAAGAUCGAAAAGAUCCUGAC |
| | | AUUCAGAAUCCCGUACUACGUCGGACCGCUGGCAAGAGGAAACA |
| | | GCAGAUUCGCAUGGAUGACAAGAAAGAGCGAAGAAACAAUCACA |
| | | CCGUGGAACUUCGAAGAAGUCGUCGACAAGGGAGCAAGCGCACA |
| | | GAGCUUCAUCGAAAGAAUGACAAACUUCGACAAGAACCUGCCGA |
| | | ACGAAAAGGUCCUGCCGAAGCACAGCCUGCUGUACGAAUACUUCA |
| | | CAGUCUACAACGAACUGACAAAGGUCAAGUACGUCACAGAAGGA |
| | | AUGAGAAAGCCGGCAUUCCUGAGCGGAGAACAGAAGAAGGCAAU |
| | | CGUCGACCUGCUGUUCAAGACAAACAGAAAGGUCACAGUCAAGC |
| | | AGCUGAAGGAAGACUACUUCAAGAAGAUCGAAUGCUUCGACAGC |
| | | GUCGAAAUCAGCGGAGUCGAAGACAGAUUCAACGCAAGCCUGGG |
| | | AACAUACCACGACCUGCUGAAGAUCAUCAAGGACAAGGACUUCCU |
| | | GGACAACGAAGAAAACGAAGACAUCCUGGAAGACAUCGUCCUGA |
| | | CACUGACACUGUUCGAAGACAGAGAAAUGAUCGAAGAAAGACUG |
| | | AAGACAUACGCACACCUGUUCGACGACAAGGUCAUGAAGCAGCU |
| | | GAAGAGAAGAAGAUACACAGGAUGGGGAAGACUGAGCAGAAAGC |
| | | UGAUCAACGGAAUCAGAGACAAGCAGAGCGGAAAGACAAUCCUG |
| | | GACUUCCUGAAGAGCGACGGAUUCGCAAACAGAAACUUCAUGCA |
| | | GCUGAUCCACGACGACAGCCUGACAUUCAAGGAAGACAUCCAGAA |
| | | GGCACAGGUCAGCGGACAGGGAGACAGCCUGCACGAACACAUCGC |
| | | AAACCUGGCAGGAAGCCCGGCAAUCAAGAAGGGAAUCCUGCAGA |
| | | CAGUCAAGGUCGUCGACGAACUGGUCAAGGUCAUGGGAAGACAC |
| | | AAGCCGGAAAACAUCGUCAUCGAAAUGGCAAGAGAAAACCAGAC |
| | | AACACAGAAGGGACAGAAGAACAGCAGAGAAAGAAUGAAGAGAA |
| | | UCGAAGAAGGAAUCAAGGAACUGGGAAGCCAGAUCCUGAAGGAA |
| | | CACCCGGUCGAAAACACACAGCUGCAGAACGAAAAGCUGUACCUG |
| | | UACUACCUGCAGAACGGAAGAGACAUGUACGUCGACCAGGAACU |
| | | GGACAUCAACAGACUGAGCGACUACGACGUCGACCACAUCGUCCC |
| | | GCAGAGCUUCCUGAAGGACGACAGCAUCGACAACAAGGUCCUGAC |
| | | AAGAAGCGACAAGAACAGAGGAAAGAGCGACAACGUCCCGAGCG |
| | | AAGAAGUCGUCAAGAAGAUGAAGAACUACUGGAGACAGCUGCUG |
| | | AACGCAAAGCUGAUCACACAGAGAAAGUUCGACAACCUGACAAA |
| | | GGCAGAGAGAGGAGGACUGAGCGAACUGGACAAGGCAGGAUUCA |
| | | UCAAGAGACAGCUGGUCGAAACAAGACAGAUCACAAAGCACGUC |
| | | GCACAGAUCCUGGACAGCAGAAUGAACACAAAGUACGACGAAAA |
| | | CGACAAGCUGAUCAGAGAAGUCAAGGUCAUCACACUGAAGAGCA |
| | | AGCUGGUCAGCGACUUCAGAAAGGACUUCCAGUUCUACAAGGUC |
| | | AGAGAAAUCAACAACUACCACCACGCACACGACGCAUACCUGAAC |
| | | GCAGUCGUCGGAACAGCACUGAUCAAGAAGUACCCGAAGCUGGA |
| | | AAGCGAAUUCGUCUACGGAGACUACAAGGUCUACGACGUCAGAA |
| | | AGAUGAUCGCAAAGAGCGAACAGGAAAUCGGAAAGGCAACAGCA |
| | | AAGUACUUCUUCUACAGCAACAUCAUGAACUUCUUCAAGACAGA |
| | | AAUCACACUGGCAAACGGAGAAAUCAGAAAGAGACCGCUGAUCG |
| | | AAACAAACGGAGAAACAGGAGAAAUCGUCUGGGACAAGGGAAGA |
| | | GACUUCGCAACAGUCAGAAAGGUCCUGAGCAUGCCGCAGGUCAAC |
| | | AUCGUCAAGAAGACAGAAGUCCAGACAGGAGGAUUCAGCAAGGA |
| | | AAGCAUCCUGCCGAAGAGAAACAGCGACAAGCUGAUCGCAAGAA |
| | | AGAAGGACUGGGACCCGAAGAAGUACGGAGGAUUCGACAGCCCG |
| | | ACAGUCGCAUACAGCGUCCUGGUCGUCGCAAAGGUCGAAAAGGG |
| | | AAAGAGCAAGAAGCUGAAGAGCGUCAAGGAACUGCUGGGAAUCA |
| | | CAAUCAUGGAAAGAAGCAGCUUCGAAAAGAACCCGAUCGACUUC |
| | | CUGGAAGCAAAGGGAUACAAGGAAGUCAAGAAGGACCUGAUCAU |
| | | CAAGCUGCCGAAGUACAGCCUGUUCGAACUGGAAAACGGAAGAA |
| | | AGAGAAUGCUGGCAAGCGCAGGAGAACUGCAGAAGGGAAACGAA |
| | | CUGGCACUGCCGAGCAAGUACGUCAACUUCCUGUACCUGGCAAGC |
| | | CACUACGAAAAGCUGAAGGGAAGCCCGGAAGACAACGAACAGAA |
| | | GCAGCUGUUCGUCGAACAGCACAAGCACUACCUGGACGAAAUCAU |
| | | CGAACAGAUCAGCGAAUUCAGCAAGAGAGUCAUCCUGGCAGACG |
| | | CAAACCUGGACAAGGUCCUGAGCGCAUACAACAAGCACAGAGACA |
| | | AGCCGAUCAGAGAACAGGCAGAAAACAUCAUCCACCUGUUCACAC |
| | | UGACAAACCUGGGAGCACCGGCAGCAUUCAAGUACUUCGACACAA |
| | | CAAUCGACAGAAAGAGAUACACAAGCACAAAGGAAGUCCUGGAC |
| | | GCAACACUGAUCCACCAGAGCAUCACAGGACUGUACGAAACAAGA |
| | | AUCGACCUGAGCCAGCUGGGAGGAGAC |
| | | GGAAGCGGAAGCCCGAAGAAGAAGAGAAAGGUCGACGGAAGCCC |
| | | GAAGAAGAAGAGAAAGGUCGACAGCGGA |
| 47 | Cas9 nickase | ATGGACAAGAAGTACTCCATCGGCCTGGCCATCGGCACCAACTCCG |
| | ORF using low A | TGGGCTGGGCCGTGATCACCGACGAGTACAAGGTGCCCTCCAAGAA |
| | codons of Table | GTTCAAGGTGCTGGGCAACACCGACCGGCACTCCATCAAGAAGAAC |
| | 4, with start and | CTGATCGGCGCCCTGCTGTTCGACTCCGGCGAGACCGCCGAGGCCA |
| | stop codons | CCCGGCTGAAGCGGACCGCCCGGCGGCGGTACACCCGGCGGAAGA |
| | | ACCGGATCTGCTACCTGCAGGAGATCTTCTCCAACGAGATGGCCAA |
| | | GGTGGACGACTCCTTCTTCCACCGGCTGGAGGAGTCCTTCCTGGTG |
| | | GAGGAGGACAAGAAGCACGAGCGGCACCCCATCTTCGGCAACATC |
| | | GTGGACGAGGTGGCCTACCACGAGAAGTACCCCACCATCTACCACC |
| | | TGCGGAAGAAGCTGGTGGACTCCACCGACAAGGCCGACCTGCGGCT |
| | | GATCTACCTGGCCCTGGCCCACATGATCAAGTTCCGGGGCCACTTC |
| | | CTGATCGAGGGCGACCTGAACCCCGACAACTCCGACGTGGACAAGC |
| | | TGTTCATCCAGCTGGTGCAGACCTACAACCAGCTGTTCGAGGAGAA |
| | | CCCCATCAACGCCTCCGGCGTGGACGCCAAGGCCATCCTGTCCGCC |
| | | CGGCTGTCCAAGTCCCGGCGGCTGGAGAACCTGATCGCCCAGCTGC |
| | | CCGGCGAGAAGAAGAACGGCCTGTTCGGCAACCTGATCGCCCTGTC |
| | | CCTGGGCCTGACCCCCAACTTCAAGTCCAACTTCGACCTGGCCGAG |
| | | GACGCCAAGCTGCAGCTGTCCAAGGACACCTACGACGACGACCTGG |
| | | ACAACCTGCTGGCCCAGATCGGCGACCAGTACGCCGACCTGTTCCT |
| | | GGCCGCCAAGAACCTGTCCGACGCCATCCTGCTGTCCGACATCCTG |
| | | CGGGTGAACACCGAGATCACCAAGGCCCCCCTGTCCGCCTCCATGA |
| | | TCAAGCGGTACGACGAGCACCACCAGGACCTGACCCTGCTGAAGGC |
| | | CCTGGTGCGGCAGCAGCTGCCCGAGAAGTACAAGGAGATCTTCTTC |
| | | GACCAGTCCAAGAACGGCTACGCCGGCTACATCGACGGCGGCGCCT |
| | | CCCAGGAGGAGTTCTACAAGTTCATCAAGCCCATCCTGGAGAAGAT |
| | | GGACGGCACCGAGGAGCTGCTGGTGAAGCTGAACCGGGAGGACCT |
| | | GCTGCGGAAGCAGCGGACCTTCGACAACGGCTCCATCCCCCACCAG |
| | | ATCCACCTGGGCGAGCTGCACGCCATCCTGCGGCGGCAGGAGGACT |
| | | TCTACCCCTTCCTGAAGGACAACCGGGAGAAGATCGAGAAGATCCT |
| | | GACCTTCCGGATCCCCTACTACGTGGGCCCCCTGGCCCGGGGCAAC |
| | | TCCCGGTTCGCCTGGATGACCCGGAAGTCCGAGGAGACCATCACCC |
| | | CCTGGAACTTCGAGGAGGTGGTGGACAAGGGCGCCTCCGCCCAGTC |
| | | CTTCATCGAGCGGATGACCAACTTCGACAAGAACCTGCCCAACGAG |
| | | AAGGTGCTGCCCAAGCACTCCCTGCTGTACGAGTACTTCACCGTGT |
| | | ACAACGAGCTGACCAAGGTGAAGTACGTGACCGAGGGCATGCGGA |
| | | AGCCCGCCTTCCTGTCCGGCGAGCAGAAGAAGGCCATCGTGGACCT |
| | | GCTGTTCAAGACCAACCGGAAGGTGACCGTGAAGCAGCTGAAGGA |
| | | GGACTACTTCAAGAAGATCGAGTGCTTCGACTCCGTGGAGATCTCC |
| | | GGCGTGGAGGACCGGTTCAACGCCTCCCTGGGCACCTACCACGACC |
| | | TGCTGAAGATCATCAAGGACAAGGACTTCCTGGACAACGAGGAGA |
| | | ACGAGGACATCCTGGAGGACATCGTGCTGACCCTGACCCTGTTCGA |
| | | GGACCGGGAGATGATCGAGGAGCGGCTGAAGACCTACGCCCACCT |
| | | GTTCGACGACAAGGTGATGAAGCAGCTGAAGCGGCGGCGGTACAC |
| | | CGGCTGGGGCCGGCTGTCCCGGAAGCTGATCAACGGCATCCGGGAC |
| | | AAGCAGTCCGGCAAGACCATCCTGGACTTCCTGAAGTCCGACGGCT |
| | | TCGCCAACCGGAACTTCATGCAGCTGATCCACGACGACTCCCTGAC |
| | | CTTCAAGGAGGACATCCAGAAGGCCCAGGTGTCCGGCCAGGGCGA |
| | | CTCCCTGCACGAGCACATCGCCAACCTGGCCGGCTCCCCCGCCATC |
| | | AAGAAGGGCATCCTGCAGACCGTGAAGGTGGTGGACGAGCTGGTG |
| | | AAGGTGATGGGCCGGCACAAGCCCGAGAACATCGTGATCGAGATG |
| | | GCCCGGGAGAACCAGACCACCCAGAAGGGCCAGAAGAACTCCCGG |
| | | GAGCGGATGAAGCGGATCGAGGAGGGCATCAAGGAGCTGGGCTCC |
| | | CAGATCCTGAAGGAGCACCCCGTGGAGAACACCCAGCTGCAGAAC |
| | | GAGAAGCTGTACCTGTACTACCTGCAGAACGGCCGGGACATGTACG |
| | | TGGACCAGGAGCTGGACATCAACCGGCTGTCCGACTACGACGTGGA |
| | | CCACATCGTGCCCCAGTCCTTCCTGAAGGACGACTCCATCGACAAC |
| | | AAGGTGCTGACCCGGTCCGACAAGAACCGGGGCAAGTCCGACAAC |
| | | GTGCCCTCCGAGGAGGTGGTGAAGAAGATGAAGAACTACTGGCGG |
| | | CAGCTGCTGAACGCCAAGCTGATCACCCAGCGGAAGTTCGACAACC |
| | | TGACCAAGGCCGAGCGGGGCGGCCTGTCCGAGCTGGACAAGGCCG |
| | | GCTTCATCAAGCGGCAGCTGGTGGAGACCCGGCAGATCACCAAGCA |
| | | CGTGGCCCAGATCCTGGACTCCCGGATGAACACCAAGTACGACGAG |
| | | AACGACAAGCTGATCCGGGAGGTGAAGGTGATCACCCTGAAGTCC |
| | | AAGCTGGTGTCCGACTTCCGGAAGGACTTCCAGTTCTACAAGGTGC |
| | | GGGAGATCAACAACTACCACCACGCCCACGACGCCTACCTGAACGC |
| | | CGTGGTGGGCACCGCCCTGATCAAGAAGTACCCCAAGCTGGAGTCC |
| | | GAGTTCGTGTACGGCGACTACAAGGTGTACGACGTGCGGAAGATGA |
| | | TCGCCAAGTCCGAGCAGGAGATCGGCAAGGCCACCGCCAAGTACTT |
| | | CTTCTACTCCAACATCATGAACTTCTTCAAGACCGAGATCACCCTGG |
| | | CCAACGGCGAGATCCGGAAGCGGCCCCTGATCGAGACCAACGGCG |
| | | AGACCGGCGAGATCGTGTGGGACAAGGGCCGGGACTTCGCCACCG |
| | | TGCGGAAGGTGCTGTCCATGCCCCAGGTGAACATCGTGAAGAAGAC |
| | | CGAGGTGCAGACCGGCGGCTTCTCCAAGGAGTCCATCCTGCCCAAG |
| | | CGGAACTCCGACAAGCTGATCGCCCGGAAGAAGGACTGGGACCCC |
| | | AAGAAGTACGGCGGCTTCGACTCCCCCACCGTGGCCTACTCCGTGC |
| | | TGGTGGTGGCCAAGGTGGAGAAGGGCAAGTCCAAGAAGCTGAAGT |
| | | CCGTGAAGGAGCTGCTGGGCATCACCATCATGGAGCGGTCCTCCTT |
| | | CGAGAAGAACCCCATCGACTTCCTGGAGGCCAAGGGCTACAAGGA |
| | | GGTGAAGAAGGACCTGATCATCAAGCTGCCCAAGTACTCCCTGTTC |
| | | GAGCTGGAGAACGGCCGGAAGCGGATGCTGGCCTCCGCCGGCGAG |
| | | CTGCAGAAGGGCAACGAGCTGGCCCTGCCCTCCAAGTACGTGAACT |
| | | TCCTGTACCTGGCCTCCCACTACGAGAAGCTGAAGGGCTCCCCCGA |
| | | GGACAACGAGCAGAAGCAGCTGTTCGTGGAGCAGCACAAGCACTA |
| | | CCTGGACGAGATCATCGAGCAGATCTCCGAGTTCTCCAAGCGGGTG |
| | | ATCCTGGCCGACGCCAACCTGGACAAGGTGCTGTCCGCCTACAACA |
| | | AGCACCGGGACAAGCCCATCCGGGAGCAGGCCGAGAACATCATCC |
| | | ACCTGTTCACCCTGACCAACCTGGGCGCCCCCGCCGCCTTCAAGTA |
| | | CTTCGACACCACCATCGACCGGAAGCGGTACACCTCCACCAAGGAG |
| | | GTGCTGGACGCCACCCTGATCCACCAGTCCATCACCGGCCTGTACG |
| | | AGACCCGGATCGACCTGTCCCAGCTGGGCGGCGACGGCGGCGGCTC |
| | | CCCCAAGAAGAAGCGGAAGGTGTGA |
| 48 | Cas9 nickase | ATGGACAAGAAGTACTCCATCGGCCTGGCCATCGGCACCAACTCCG |
| | ORF using low A | TGGGCTGGGCCGTGATCACCGACGAGTACAAGGTGCCCTCCAAGAA |
| | codons of Table | GTTCAAGGTGCTGGGCAACACCGACCGGCACTCCATCAAGAAGAAC |
| | 4, with start and | CTGATCGGCGCCCTGCTGTTCGACTCCGGCGAGACCGCCGAGGCCA |
| | stop codons and | CCCGGCTGAAGCGGACCGCCCGGCGGCGGTACACCCGGCGGAAGA |
| | no NLS | ACCGGATCTGCTACCTGCAGGAGATCTTCTCCAACGAGATGGCCAA |
| | | GGTGGACGACTCCTTCTTCCACCGGCTGGAGGAGTCCTTCCTGGTG |
| | | GAGGAGGACAAGAAGCACGAGCGGCACCCCATCTTCGGCAACATC |
| | | GTGGACGAGGTGGCCTACCACGAGAAGTACCCCACCATCTACCACC |
| | | TGCGGAAGAAGCTGGTGGACTCCACCGACAAGGCCGACCTGCGGCT |
| | | GATCTACCTGGCCCTGGCCCACATGATCAAGTTCCGGGGCCACTTC |
| | | CTGATCGAGGGCGACCTGAACCCCGACAACTCCGACGTGGACAAGC |
| | | TGTTCATCCAGCTGGTGCAGACCTACAACCAGCTGTTCGAGGAGAA |
| | | CCCCATCAACGCCTCCGGCGTGGACGCCAAGGCCATCCTGTCCGCC |
| | | CGGCTGTCCAAGTCCCGGCGGCTGGAGAACCTGATCGCCCAGCTGC |
| | | CCGGCGAGAAGAAGAACGGCCTGTTCGGCAACCTGATCGCCCTGTC |
| | | CCTGGGCCTGACCCCCAACTTCAAGTCCAACTTCGACCTGGCCGAG |
| | | GACGCCAAGCTGCAGCTGTCCAAGGACACCTACGACGACGACCTGG |
| | | ACAACCTGCTGGCCCAGATCGGCGACCAGTACGCCGACCTGTTCCT |
| | | GGCCGCCAAGAACCTGTCCGACGCCATCCTGCTGTCCGACATCCTG |
| | | CGGGTGAACACCGAGATCACCAAGGCCCCCCTGTCCGCCTCCATGA |
| | | TCAAGCGGTACGACGAGCACCACCAGGACCTGACCCTGCTGAAGGC |
| | | CCTGGTGCGGCAGCAGCTGCCCGAGAAGTACAAGGAGATCTTCTTC |
| | | GACCAGTCCAAGAACGGCTACGCCGGCTACATCGACGGCGGCGCCT |
| | | CCCAGGAGGAGTTCTACAAGTTCATCAAGCCCATCCTGGAGAAGAT |
| | | GGACGGCACCGAGGAGCTGCTGGTGAAGCTGAACCGGGAGGACCT |
| | | GCTGCGGAAGCAGCGGACCTTCGACAACGGCTCCATCCCCCACCAG |
| | | ATCCACCTGGGCGAGCTGCACGCCATCCTGCGGCGGCAGGAGGACT |
| | | TCTACCCCTTCCTGAAGGACAACCGGGAGAAGATCGAGAAGATCCT |
| | | GACCTTCCGGATCCCCTACTACGTGGGCCCCCTGGCCCGGGGCAAC |
| | | TCCCGGTTCGCCTGGATGACCCGGAAGTCCGAGGAGACCATCACCC |
| | | CCTGGAACTTCGAGGAGGTGGTGGACAAGGGCGCCTCCGCCCAGTC |
| | | CTTCATCGAGCGGATGACCAACTTCGACAAGAACCTGCCCAACGAG |
| | | AAGGTGCTGCCCAAGCACTCCCTGCTGTACGAGTACTTCACCGTGT |
| | | ACAACGAGCTGACCAAGGTGAAGTACGTGACCGAGGGCATGCGGA |
| | | AGCCCGCCTTCCTGTCCGGCGAGCAGAAGAAGGCCATCGTGGACCT |
| | | GCTGTTCAAGACCAACCGGAAGGTGACCGTGAAGCAGCTGAAGGA |
| | | GGACTACTTCAAGAAGATCGAGTGCTTCGACTCCGTGGAGATCTCC |
| | | GGCGTGGAGGACCGGTTCAACGCCTCCCTGGGCACCTACCACGACC |
| | | TGCTGAAGATCATCAAGGACAAGGACTTCCTGGACAACGAGGAGA |
| | | ACGAGGACATCCTGGAGGACATCGTGCTGACCCTGACCCTGTTCGA |
| | | GGACCGGGAGATGATCGAGGAGCGGCTGAAGACCTACGCCCACCT |
| | | GTTCGACGACAAGGTGATGAAGCAGCTGAAGCGGCGGCGGTACAC |
| | | CGGCTGGGGCCGGCTGTCCCGGAAGCTGATCAACGGCATCCGGGAC |
| | | AAGCAGTCCGGCAAGACCATCCTGGACTTCCTGAAGTCCGACGGCT |
| | | TCGCCAACCGGAACTTCATGCAGCTGATCCACGACGACTCCCTGAC |
| | | CTTCAAGGAGGACATCCAGAAGGCCCAGGTGTCCGGCCAGGGCGA |
| | | CTCCCTGCACGAGCACATCGCCAACCTGGCCGGCTCCCCCGCCATC |
| | | AAGAAGGGCATCCTGCAGACCGTGAAGGTGGTGGACGAGCTGGTG |
| | | AAGGTGATGGGCCGGCACAAGCCCGAGAACATCGTGATCGAGATG |
| | | GCCCGGGAGAACCAGACCACCCAGAAGGGCCAGAAGAACTCCCGG |
| | | GAGCGGATGAAGCGGATCGAGGAGGGCATCAAGGAGCTGGGCTCC |
| | | CAGATCCTGAAGGAGCACCCCGTGGAGAACACCCAGCTGCAGAAC |
| | | GAGAAGCTGTACCTGTACTACCTGCAGAACGGCCGGGACATGTACG |
| | | TGGACCAGGAGCTGGACATCAACCGGCTGTCCGACTACGACGTGGA |
| | | CCACATCGTGCCCCAGTCCTTCCTGAAGGACGACTCCATCGACAAC |
| | | AAGGTGCTGACCCGGTCCGACAAGAACCGGGGCAAGTCCGACAAC |
| | | GTGCCCTCCGAGGAGGTGGTGAAGAAGATGAAGAACTACTGGCGG |
| | | CAGCTGCTGAACGCCAAGCTGATCACCCAGCGGAAGTTCGACAACC |
| | | TGACCAAGGCCGAGCGGGGCGGCCTGTCCGAGCTGGACAAGGCCG |
| | | GCTTCATCAAGCGGCAGCTGGTGGAGACCCGGCAGATCACCAAGCA |
| | | CGTGGCCCAGATCCTGGACTCCCGGATGAACACCAAGTACGACGAG |
| | | AACGACAAGCTGATCCGGGAGGTGAAGGTGATCACCCTGAAGTCC |
| | | AAGCTGGTGTCCGACTTCCGGAAGGACTTCCAGTTCTACAAGGTGC |
| | | GGGAGATCAACAACTACCACCACGCCCACGACGCCTACCTGAACGC |
| | | CGTGGTGGGCACCGCCCTGATCAAGAAGTACCCCAAGCTGGAGTCC |
| | | GAGTTCGTGTACGGCGACTACAAGGTGTACGACGTGCGGAAGATGA |
| | | TCGCCAAGTCCGAGCAGGAGATCGGCAAGGCCACCGCCAAGTACTT |
| | | CTTCTACTCCAACATCATGAACTTCTTCAAGACCGAGATCACCCTGG |
| | | CCAACGGCGAGATCCGGAAGCGGCCCCTGATCGAGACCAACGGCG |
| | | AGACCGGCGAGATCGTGTGGGACAAGGGCCGGGACTTCGCCACCG |
| | | TGCGGAAGGTGCTGTCCATGCCCCAGGTGAACATCGTGAAGAAGAC |
| | | CGAGGTGCAGACCGGCGGCTTCTCCAAGGAGTCCATCCTGCCCAAG |
| | | CGGAACTCCGACAAGCTGATCGCCCGGAAGAAGGACTGGGACCCC |
| | | AAGAAGTACGGCGGCTTCGACTCCCCCACCGTGGCCTACTCCGTGC |
| | | TGGTGGTGGCCAAGGTGGAGAAGGGCAAGTCCAAGAAGCTGAAGT |
| | | CCGTGAAGGAGCTGCTGGGCATCACCATCATGGAGCGGTCCTCCTT |
| | | CGAGAAGAACCCCATCGACTTCCTGGAGGCCAAGGGCTACAAGGA |
| | | GGTGAAGAAGGACCTGATCATCAAGCTGCCCAAGTACTCCCTGTTC |
| | | GAGCTGGAGAACGGCCGGAAGCGGATGCTGGCCTCCGCCGGCGAG |
| | | CTGCAGAAGGGCAACGAGCTGGCCCTGCCCTCCAAGTACGTGAACT |
| | | TCCTGTACCTGGCCTCCCACTACGAGAAGCTGAAGGGCTCCCCCGA |
| | | GGACAACGAGCAGAAGCAGCTGTTCGTGGAGCAGCACAAGCACTA |
| | | CCTGGACGAGATCATCGAGCAGATCTCCGAGTTCTCCAAGCGGGTG |
| | | ATCCTGGCCGACGCCAACCTGGACAAGGTGCTGTCCGCCTACAACA |
| | | AGCACCGGGACAAGCCCATCCGGGAGCAGGCCGAGAACATCATCC |
| | | ACCTGTTCACCCTGACCAACCTGGGCGCCCCCGCCGCCTTCAAGTA |
| | | CTTCGACACCACCATCGACCGGAAGCGGTACACCTCCACCAAGGAG |
| | | GTGCTGGACGCCACCCTGATCCACCAGTCCATCACCGGCCTGTACG |
| | | AGACCCGGATCGACCTGTCCCAGCTGGGCGGCGACTGA |
| 49 | Cas9 nickase | ATGGACAAGAAGTACTCCATCGGCCTGGCCATCGGCACCAACTCCG |
| | ORF using low A | TGGGCTGGGCCGTGATCACCGACGAGTACAAGGTGCCCTCCAAGAA |
| | codons of Table | GTTCAAGGTGCTGGGCAACACCGACCGGCACTCCATCAAGAAGAAC |
| | 4, with two C- | CTGATCGGCGCCCTGCTGTTCGACTCCGGCGAGACCGCCGAGGCCA |
| | terminal NLS | CCCGGCTGAAGCGGACCGCCCGGCGGCGGTACACCCGGCGGAAGA |
| | sequences and | ACCGGATCTGCTACCTGCAGGAGATCTTCTCCAACGAGATGGCCAA |
| | start and stop | GGTGGACGACTCCTTCTTCCACCGGCTGGAGGAGTCCTTCCTGGTG |
| | codons | GAGGAGGACAAGAAGCACGAGCGGCACCCCATCTTCGGCAACATC |
| | | GTGGACGAGGTGGCCTACCACGAGAAGTACCCCACCATCTACCACC |
| | | TGCGGAAGAAGCTGGTGGACTCCACCGACAAGGCCGACCTGCGGCT |
| | | GATCTACCTGGCCCTGGCCCACATGATCAAGTTCCGGGGCCACTTC |
| | | CTGATCGAGGGCGACCTGAACCCCGACAACTCCGACGTGGACAAGC |
| | | TGTTCATCCAGCTGGTGCAGACCTACAACCAGCTGTTCGAGGAGAA |
| | | CCCCATCAACGCCTCCGGCGTGGACGCCAAGGCCATCCTGTCCGCC |
| | | CGGCTGTCCAAGTCCCGGCGGCTGGAGAACCTGATCGCCCAGCTGC |
| | | CCGGCGAGAAGAAGAACGGCCTGTTCGGCAACCTGATCGCCCTGTC |
| | | CCTGGGCCTGACCCCCAACTTCAAGTCCAACTTCGACCTGGCCGAG |
| | | GACGCCAAGCTGCAGCTGTCCAAGGACACCTACGACGACGACCTGG |
| | | ACAACCTGCTGGCCCAGATCGGCGACCAGTACGCCGACCTGTTCCT |
| | | GGCCGCCAAGAACCTGTCCGACGCCATCCTGCTGTCCGACATCCTG |
| | | CGGGTGAACACCGAGATCACCAAGGCCCCCCTGTCCGCCTCCATGA |
| | | TCAAGCGGTACGACGAGCACCACCAGGACCTGACCCTGCTGAAGGC |
| | | CCTGGTGCGGCAGCAGCTGCCCGAGAAGTACAAGGAGATCTTCTTC |
| | | GACCAGTCCAAGAACGGCTACGCCGGCTACATCGACGGCGGCGCCT |
| | | CCCAGGAGGAGTTCTACAAGTTCATCAAGCCCATCCTGGAGAAGAT |
| | | GGACGGCACCGAGGAGCTGCTGGTGAAGCTGAACCGGGAGGACCT |
| | | GCTGCGGAAGCAGCGGACCTTCGACAACGGCTCCATCCCCCACCAG |
| | | ATCCACCTGGGCGAGCTGCACGCCATCCTGCGGCGGCAGGAGGACT |
| | | TCTACCCCTTCCTGAAGGACAACCGGGAGAAGATCGAGAAGATCCT |
| | | GACCTTCCGGATCCCCTACTACGTGGGCCCCCTGGCCCGGGGCAAC |
| | | TCCCGGTTCGCCTGGATGACCCGGAAGTCCGAGGAGACCATCACCC |
| | | CCTGGAACTTCGAGGAGGTGGTGGACAAGGGCGCCTCCGCCCAGTC |
| | | CTTCATCGAGCGGATGACCAACTTCGACAAGAACCTGCCCAACGAG |
| | | AAGGTGCTGCCCAAGCACTCCCTGCTGTACGAGTACTTCACCGTGT |
| | | ACAACGAGCTGACCAAGGTGAAGTACGTGACCGAGGGCATGCGGA |
| | | AGCCCGCCTTCCTGTCCGGCGAGCAGAAGAAGGCCATCGTGGACCT |
| | | GCTGTTCAAGACCAACCGGAAGGTGACCGTGAAGCAGCTGAAGGA |
| | | GGACTACTTCAAGAAGATCGAGTGCTTCGACTCCGTGGAGATCTCC |
| | | GGCGTGGAGGACCGGTTCAACGCCTCCCTGGGCACCTACCACGACC |
| | | TGCTGAAGATCATCAAGGACAAGGACTTCCTGGACAACGAGGAGA |
| | | ACGAGGACATCCTGGAGGACATCGTGCTGACCCTGACCCTGTTCGA |
| | | GGACCGGGAGATGATCGAGGAGCGGCTGAAGACCTACGCCCACCT |
| | | GTTCGACGACAAGGTGATGAAGCAGCTGAAGCGGCGGCGGTACAC |
| | | CGGCTGGGGCCGGCTGTCCCGGAAGCTGATCAACGGCATCCGGGAC |
| | | AAGCAGTCCGGCAAGACCATCCTGGACTTCCTGAAGTCCGACGGCT |
| | | TCGCCAACCGGAACTTCATGCAGCTGATCCACGACGACTCCCTGAC |
| | | CTTCAAGGAGGACATCCAGAAGGCCCAGGTGTCCGGCCAGGGCGA |
| | | CTCCCTGCACGAGCACATCGCCAACCTGGCCGGCTCCCCCGCCATC |
| | | AAGAAGGGCATCCTGCAGACCGTGAAGGTGGTGGACGAGCTGGTG |
| | | AAGGTGATGGGCCGGCACAAGCCCGAGAACATCGTGATCGAGATG |
| | | GCCCGGGAGAACCAGACCACCCAGAAGGGCCAGAAGAACTCCCGG |
| | | GAGCGGATGAAGCGGATCGAGGAGGGCATCAAGGAGCTGGGCTCC |
| | | CAGATCCTGAAGGAGCACCCCGTGGAGAACACCCAGCTGCAGAAC |
| | | GAGAAGCTGTACCTGTACTACCTGCAGAACGGCCGGGACATGTACG |
| | | TGGACCAGGAGCTGGACATCAACCGGCTGTCCGACTACGACGTGGA |
| | | CCACATCGTGCCCCAGTCCTTCCTGAAGGACGACTCCATCGACAAC |
| | | AAGGTGCTGACCCGGTCCGACAAGAACCGGGGCAAGTCCGACAAC |
| | | GTGCCCTCCGAGGAGGTGGTGAAGAAGATGAAGAACTACTGGCGG |
| | | CAGCTGCTGAACGCCAAGCTGATCACCCAGCGGAAGTTCGACAACC |
| | | TGACCAAGGCCGAGCGGGGCGGCCTGTCCGAGCTGGACAAGGCCG |
| | | GCTTCATCAAGCGGCAGCTGGTGGAGACCCGGCAGATCACCAAGCA |
| | | CGTGGCCCAGATCCTGGACTCCCGGATGAACACCAAGTACGACGAG |
| | | AACGACAAGCTGATCCGGGAGGTGAAGGTGATCACCCTGAAGTCC |
| | | AAGCTGGTGTCCGACTTCCGGAAGGACTTCCAGTTCTACAAGGTGC |
| | | GGGAGATCAACAACTACCACCACGCCCACGACGCCTACCTGAACGC |
| | | CGTGGTGGGCACCGCCCTGATCAAGAAGTACCCCAAGCTGGAGTCC |
| | | GAGTTCGTGTACGGCGACTACAAGGTGTACGACGTGCGGAAGATGA |
| | | TCGCCAAGTCCGAGCAGGAGATCGGCAAGGCCACCGCCAAGTACTT |
| | | CTTCTACTCCAACATCATGAACTTCTTCAAGACCGAGATCACCCTGG |
| | | CCAACGGCGAGATCCGGAAGCGGCCCCTGATCGAGACCAACGGCG |
| | | AGACCGGCGAGATCGTGTGGGACAAGGGCCGGGACTTCGCCACCG |
| | | TGCGGAAGGTGCTGTCCATGCCCCAGGTGAACATCGTGAAGAAGAC |
| | | CGAGGTGCAGACCGGCGGCTTCTCCAAGGAGTCCATCCTGCCCAAG |
| | | CGGAACTCCGACAAGCTGATCGCCCGGAAGAAGGACTGGGACCCC |
| | | AAGAAGTACGGCGGCTTCGACTCCCCCACCGTGGCCTACTCCGTGC |
| | | TGGTGGTGGCCAAGGTGGAGAAGGGCAAGTCCAAGAAGCTGAAGT |
| | | CCGTGAAGGAGCTGCTGGGCATCACCATCATGGAGCGGTCCTCCTT |
| | | CGAGAAGAACCCCATCGACTTCCTGGAGGCCAAGGGCTACAAGGA |
| | | GGTGAAGAAGGACCTGATCATCAAGCTGCCCAAGTACTCCCTGTTC |
| | | GAGCTGGAGAACGGCCGGAAGCGGATGCTGGCCTCCGCCGGCGAG |
| | | CTGCAGAAGGGCAACGAGCTGGCCCTGCCCTCCAAGTACGTGAACT |
| | | TCCTGTACCTGGCCTCCCACTACGAGAAGCTGAAGGGCTCCCCCGA |
| | | GGACAACGAGCAGAAGCAGCTGTTCGTGGAGCAGCACAAGCACTA |
| | | CCTGGACGAGATCATCGAGCAGATCTCCGAGTTCTCCAAGCGGGTG |
| | | ATCCTGGCCGACGCCAACCTGGACAAGGTGCTGTCCGCCTACAACA |
| | | AGCACCGGGACAAGCCCATCCGGGAGCAGGCCGAGAACATCATCC |
| | | ACCTGTTCACCCTGACCAACCTGGGCGCCCCCGCCGCCTTCAAGTA |
| | | CTTCGACACCACCATCGACCGGAAGCGGTACACCTCCACCAAGGAG |
| | | GTGCTGGACGCCACCCTGATCCACCAGTCCATCACCGGCCTGTACG |
| | | AGACCCGGATCGACCTGTCCCAGCTGGGCGGCGACGGCTCCGGCTC |
| | | CCCCAAGAAGAAGCGGAAGGTGGACGGCTCCCCCAAGAAGAAGCG |
| | | GAAGGTGGACTCCGGCTGA |
| 50 | Cas9 nickase | ATGGACAAGAAGTACAGCATCGGCCTGGCCATCGGCACCAACAGC |
| | ORF using low | GTGGGCTGGGCCGTGATCACCGACGAGTACAAGGTGCCCAGCAAG |
| | A/U codons of | AAGTTCAAGGTGCTGGGCAACACCGACCGGCACAGCATCAAGAAG |
| | Table 4, with start | AACCTGATCGGCGCCCTGCTGTTCGACAGCGGCGAGACCGCCGAGG |
| | and stop codons | CCACCCGGCTGAAGCGGACCGCCCGGCGGCGGTACACCCGGCGGA |
| | | AGAACCGGATCTGCTACCTGCAGGAGATCTTCAGCAACGAGATGGC |
| | | CAAGGTGGACGACAGCTTCTTCCACCGGCTGGAGGAGAGCTTCCTG |
| | | GTGGAGGAGGACAAGAAGCACGAGCGGCACCCCATCTTCGGCAAC |
| | | ATCGTGGACGAGGTGGCCTACCACGAGAAGTACCCCACCATCTACC |
| | | ACCTGCGGAAGAAGCTGGTGGACAGCACCGACAAGGCCGACCTGC |
| | | GGCTGATCTACCTGGCCCTGGCCCACATGATCAAGTTCCGGGGCCA |
| | | CTTCCTGATCGAGGGCGACCTGAACCCCGACAACAGCGACGTGGAC |
| | | AAGCTGTTCATCCAGCTGGTGCAGACCTACAACCAGCTGTTCGAGG |
| | | AGAACCCCATCAACGCCAGCGGCGTGGACGCCAAGGCCATCCTGA |
| | | GCGCCCGGCTGAGCAAGAGCCGGCGGCTGGAGAACCTGATCGCCC |
| | | AGCTGCCCGGCGAGAAGAAGAACGGCCTGTTCGGCAACCTGATCGC |
| | | CCTGAGCCTGGGCCTGACCCCCAACTTCAAGAGCAACTTCGACCTG |
| | | GCCGAGGACGCCAAGCTGCAGCTGAGCAAGGACACCTACGACGAC |
| | | GACCTGGACAACCTGCTGGCCCAGATCGGCGACCAGTACGCCGACC |
| | | TGTTCCTGGCCGCCAAGAACCTGAGCGACGCCATCCTGCTGAGCGA |
| | | CATCCTGCGGGTGAACACCGAGATCACCAAGGCCCCCCTGAGCGCC |
| | | AGCATGATCAAGCGGTACGACGAGCACCACCAGGACCTGACCCTGC |
| | | TGAAGGCCCTGGTGCGGCAGCAGCTGCCCGAGAAGTACAAGGAGA |
| | | TCTTCTTCGACCAGAGCAAGAACGGCTACGCCGGCTACATCGACGG |
| | | CGGCGCCAGCCAGGAGGAGTTCTACAAGTTCATCAAGCCCATCCTG |
| | | GAGAAGATGGACGGCACCGAGGAGCTGCTGGTGAAGCTGAACCGG |
| | | GAGGACCTGCTGCGGAAGCAGCGGACCTTCGACAACGGCAGCATC |
| | | CCCCACCAGATCCACCTGGGCGAGCTGCACGCCATCCTGCGGCGGC |
| | | AGGAGGACTTCTACCCCTTCCTGAAGGACAACCGGGAGAAGATCGA |
| | | GAAGATCCTGACCTTCCGGATCCCCTACTACGTGGGCCCCCTGGCC |
| | | CGGGGCAACAGCCGGTTCGCCTGGATGACCCGGAAGAGCGAGGAG |
| | | ACCATCACCCCCTGGAACTTCGAGGAGGTGGTGGACAAGGGCGCCA |
| | | GCGCCCAGAGCTTCATCGAGCGGATGACCAACTTCGACAAGAACCT |
| | | GCCCAACGAGAAGGTGCTGCCCAAGCACAGCCTGCTGTACGAGTAC |
| | | TTCACCGTGTACAACGAGCTGACCAAGGTGAAGTACGTGACCGAGG |
| | | GCATGCGGAAGCCCGCCTTCCTGAGCGGCGAGCAGAAGAAGGCCA |
| | | TCGTGGACCTGCTGTTCAAGACCAACCGGAAGGTGACCGTGAAGCA |
| | | GCTGAAGGAGGACTACTTCAAGAAGATCGAGTGCTTCGACAGCGTG |
| | | GAGATCAGCGGCGTGGAGGACCGGTTCAACGCCAGCCTGGGCACCT |
| | | ACCACGACCTGCTGAAGATCATCAAGGACAAGGACTTCCTGGACAA |
| | | CGAGGAGAACGAGGACATCCTGGAGGACATCGTGCTGACCCTGAC |
| | | CCTGTTCGAGGACCGGGAGATGATCGAGGAGCGGCTGAAGACCTA |
| | | CGCCCACCTGTTCGACGACAAGGTGATGAAGCAGCTGAAGCGGCG |
| | | GCGGTACACCGGCTGGGGCCGGCTGAGCCGGAAGCTGATCAACGG |
| | | CATCCGGGACAAGCAGAGCGGCAAGACCATCCTGGACTTCCTGAAG |
| | | AGCGACGGCTTCGCCAACCGGAACTTCATGCAGCTGATCCACGACG |
| | | ACAGCCTGACCTTCAAGGAGGACATCCAGAAGGCCCAGGTGAGCG |
| | | GCCAGGGCGACAGCCTGCACGAGCACATCGCCAACCTGGCCGGCA |
| | | GCCCCGCCATCAAGAAGGGCATCCTGCAGACCGTGAAGGTGGTGG |
| | | ACGAGCTGGTGAAGGTGATGGGCCGGCACAAGCCCGAGAACATCG |
| | | TGATCGAGATGGCCCGGGAGAACCAGACCACCCAGAAGGGCCAGA |
| | | AGAACAGCCGGGAGCGGATGAAGCGGATCGAGGAGGGCATCAAGG |
| | | AGCTGGGCAGCCAGATCCTGAAGGAGCACCCCGTGGAGAACACCC |
| | | AGCTGCAGAACGAGAAGCTGTACCTGTACTACCTGCAGAACGGCCG |
| | | GGACATGTACGTGGACCAGGAGCTGGACATCAACCGGCTGAGCGA |
| | | CTACGACGTGGACCACATCGTGCCCCAGAGCTTCCTGAAGGACGAC |
| | | AGCATCGACAACAAGGTGCTGACCCGGAGCGACAAGAACCGGGGC |
| | | AAGAGCGACAACGTGCCCAGCGAGGAGGTGGTGAAGAAGATGAAG |
| | | AACTACTGGCGGCAGCTGCTGAACGCCAAGCTGATCACCCAGCGGA |
| | | AGTTCGACAACCTGACCAAGGCCGAGCGGGGCGGCCTGAGCGAGC |
| | | TGGACAAGGCCGGCTTCATCAAGCGGCAGCTGGTGGAGACCCGGC |
| | | AGATCACCAAGCACGTGGCCCAGATCCTGGACAGCCGGATGAACA |
| | | CCAAGTACGACGAGAACGACAAGCTGATCCGGGAGGTGAAGGTGA |
| | | TCACCCTGAAGAGCAAGCTGGTGAGCGACTTCCGGAAGGACTTCCA |
| | | GTTCTACAAGGTGCGGGAGATCAACAACTACCACCACGCCCACGAC |
| | | GCCTACCTGAACGCCGTGGTGGGCACCGCCCTGATCAAGAAGTACC |
| | | CCAAGCTGGAGAGCGAGTTCGTGTACGGCGACTACAAGGTGTACGA |
| | | CGTGCGGAAGATGATCGCCAAGAGCGAGCAGGAGATCGGCAAGGC |
| | | CACCGCCAAGTACTTCTTCTACAGCAACATCATGAACTTCTTCAAGA |
| | | CCGAGATCACCCTGGCCAACGGCGAGATCCGGAAGCGGCCCCTGAT |
| | | CGAGACCAACGGCGAGACCGGCGAGATCGTGTGGGACAAGGGCCG |
| | | GGACTTCGCCACCGTGCGGAAGGTGCTGAGCATGCCCCAGGTGAAC |
| | | ATCGTGAAGAAGACCGAGGTGCAGACCGGCGGCTTCAGCAAGGAG |
| | | AGCATCCTGCCCAAGCGGAACAGCGACAAGCTGATCGCCCGGAAG |
| | | AAGGACTGGGACCCCAAGAAGTACGGCGGCTTCGACAGCCCCACC |
| | | GTGGCCTACAGCGTGCTGGTGGTGGCCAAGGTGGAGAAGGGCAAG |
| | | AGCAAGAAGCTGAAGAGCGTGAAGGAGCTGCTGGGCATCACCATC |
| | | ATGGAGCGGAGCAGCTTCGAGAAGAACCCCATCGACTTCCTGGAGG |
| | | CCAAGGGCTACAAGGAGGTGAAGAAGGACCTGATCATCAAGCTGC |
| | | CCAAGTACAGCCTGTTCGAGCTGGAGAACGGCCGGAAGCGGATGCT |
| | | GGCCAGCGCCGGCGAGCTGCAGAAGGGCAACGAGCTGGCCCTGCC |
| | | CAGCAAGTACGTGAACTTCCTGTACCTGGCCAGCCACTACGAGAAG |
| | | CTGAAGGGCAGCCCCGAGGACAACGAGCAGAAGCAGCTGTTCGTG |
| | | GAGCAGCACAAGCACTACCTGGACGAGATCATCGAGCAGATCAGC |
| | | GAGTTCAGCAAGCGGGTGATCCTGGCCGACGCCAACCTGGACAAG |
| | | GTGCTGAGCGCCTACAACAAGCACCGGGACAAGCCCATCCGGGAG |
| | | CAGGCCGAGAACATCATCCACCTGTTCACCCTGACCAACCTGGGCG |
| | | CCCCCGCCGCCTTCAAGTACTTCGACACCACCATCGACCGGAAGCG |
| | | GTACACCAGCACCAAGGAGGTGCTGGACGCCACCCTGATCCACCAG |
| | | AGCATCACCGGCCTGTACGAGACCCGGATCGACCTGAGCCAGCTGG |
| | | GCGGCGACGGCGGCGGCAGCCCCAAGAAGAAGCGGAAGGTGTGA |
| 51 | Cas9 nickase | ATGGACAAGAAGTACAGCATCGGCCTGGCCATCGGCACCAACAGC |
| | ORF using low | GTGGGCTGGGCCGTGATCACCGACGAGTACAAGGTGCCCAGCAAG |
| | A/U codons of | AAGTTCAAGGTGCTGGGCAACACCGACCGGCACAGCATCAAGAAG |
| | Table 4, with two | AACCTGATCGGCGCCCTGCTGTTCGACAGCGGCGAGACCGCCGAGG |
| | C-terminal NLS | CCACCCGGCTGAAGCGGACCGCCCGGCGGCGGTACACCCGGCGGA |
| | sequences and | AGAACCGGATCTGCTACCTGCAGGAGATCTTCAGCAACGAGATGGC |
| | start and stop | CAAGGTGGACGACAGCTTCTTCCACCGGCTGGAGGAGAGCTTCCTG |
| | codons | GTGGAGGAGGACAAGAAGCACGAGCGGCACCCCATCTTCGGCAAC |
| | | ATCGTGGACGAGGTGGCCTACCACGAGAAGTACCCCACCATCTACC |
| | | ACCTGCGGAAGAAGCTGGTGGACAGCACCGACAAGGCCGACCTGC |
| | | GGCTGATCTACCTGGCCCTGGCCCACATGATCAAGTTCCGGGGCCA |
| | | CTTCCTGATCGAGGGCGACCTGAACCCCGACAACAGCGACGTGGAC |
| | | AAGCTGTTCATCCAGCTGGTGCAGACCTACAACCAGCTGTTCGAGG |
| | | AGAACCCCATCAACGCCAGCGGCGTGGACGCCAAGGCCATCCTGA |
| | | GCGCCCGGCTGAGCAAGAGCCGGCGGCTGGAGAACCTGATCGCCC |
| | | AGCTGCCCGGCGAGAAGAAGAACGGCCTGTTCGGCAACCTGATCGC |
| | | CCTGAGCCTGGGCCTGACCCCCAACTTCAAGAGCAACTTCGACCTG |
| | | GCCGAGGACGCCAAGCTGCAGCTGAGCAAGGACACCTACGACGAC |
| | | GACCTGGACAACCTGCTGGCCCAGATCGGCGACCAGTACGCCGACC |
| | | TGTTCCTGGCCGCCAAGAACCTGAGCGACGCCATCCTGCTGAGCGA |
| | | CATCCTGCGGGTGAACACCGAGATCACCAAGGCCCCCCTGAGCGCC |
| | | AGCATGATCAAGCGGTACGACGAGCACCACCAGGACCTGACCCTGC |
| | | TGAAGGCCCTGGTGCGGCAGCAGCTGCCCGAGAAGTACAAGGAGA |
| | | TCTTCTTCGACCAGAGCAAGAACGGCTACGCCGGCTACATCGACGG |
| | | CGGCGCCAGCCAGGAGGAGTTCTACAAGTTCATCAAGCCCATCCTG |
| | | GAGAAGATGGACGGCACCGAGGAGCTGCTGGTGAAGCTGAACCGG |
| | | GAGGACCTGCTGCGGAAGCAGCGGACCTTCGACAACGGCAGCATC |
| | | CCCCACCAGATCCACCTGGGCGAGCTGCACGCCATCCTGCGGCGGC |
| | | AGGAGGACTTCTACCCCTTCCTGAAGGACAACCGGGAGAAGATCGA |
| | | GAAGATCCTGACCTTCCGGATCCCCTACTACGTGGGCCCCCTGGCC |
| | | CGGGGCAACAGCCGGTTCGCCTGGATGACCCGGAAGAGCGAGGAG |
| | | ACCATCACCCCCTGGAACTTCGAGGAGGTGGTGGACAAGGGCGCCA |
| | | GCGCCCAGAGCTTCATCGAGCGGATGACCAACTTCGACAAGAACCT |
| | | GCCCAACGAGAAGGTGCTGCCCAAGCACAGCCTGCTGTACGAGTAC |
| | | TTCACCGTGTACAACGAGCTGACCAAGGTGAAGTACGTGACCGAGG |
| | | GCATGCGGAAGCCCGCCTTCCTGAGCGGCGAGCAGAAGAAGGCCA |
| | | TCGTGGACCTGCTGTTCAAGACCAACCGGAAGGTGACCGTGAAGCA |
| | | GCTGAAGGAGGACTACTTCAAGAAGATCGAGTGCTTCGACAGCGTG |
| | | GAGATCAGCGGCGTGGAGGACCGGTTCAACGCCAGCCTGGGCACCT |
| | | ACCACGACCTGCTGAAGATCATCAAGGACAAGGACTTCCTGGACAA |
| | | CGAGGAGAACGAGGACATCCTGGAGGACATCGTGCTGACCCTGAC |
| | | CCTGTTCGAGGACCGGGAGATGATCGAGGAGCGGCTGAAGACCTA |
| | | CGCCCACCTGTTCGACGACAAGGTGATGAAGCAGCTGAAGCGGCG |
| | | GCGGTACACCGGCTGGGGCCGGCTGAGCCGGAAGCTGATCAACGG |
| | | CATCCGGGACAAGCAGAGCGGCAAGACCATCCTGGACTTCCTGAAG |
| | | AGCGACGGCTTCGCCAACCGGAACTTCATGCAGCTGATCCACGACG |
| | | ACAGCCTGACCTTCAAGGAGGACATCCAGAAGGCCCAGGTGAGCG |
| | | GCCAGGGCGACAGCCTGCACGAGCACATCGCCAACCTGGCCGGCA |
| | | GCCCCGCCATCAAGAAGGGCATCCTGCAGACCGTGAAGGTGGTGG |
| | | ACGAGCTGGTGAAGGTGATGGGCCGGCACAAGCCCGAGAACATCG |
| | | TGATCGAGATGGCCCGGGAGAACCAGACCACCCAGAAGGGCCAGA |
| | | AGAACAGCCGGGAGCGGATGAAGCGGATCGAGGAGGGCATCAAGG |
| | | AGCTGGGCAGCCAGATCCTGAAGGAGCACCCCGTGGAGAACACCC |
| | | AGCTGCAGAACGAGAAGCTGTACCTGTACTACCTGCAGAACGGCCG |
| | | GGACATGTACGTGGACCAGGAGCTGGACATCAACCGGCTGAGCGA |
| | | CTACGACGTGGACCACATCGTGCCCCAGAGCTTCCTGAAGGACGAC |
| | | AGCATCGACAACAAGGTGCTGACCCGGAGCGACAAGAACCGGGGC |
| | | AAGAGCGACAACGTGCCCAGCGAGGAGGTGGTGAAGAAGATGAAG |
| | | AACTACTGGCGGCAGCTGCTGAACGCCAAGCTGATCACCCAGCGGA |
| | | AGTTCGACAACCTGACCAAGGCCGAGCGGGGCGGCCTGAGCGAGC |
| | | TGGACAAGGCCGGCTTCATCAAGCGGCAGCTGGTGGAGACCCGGC |
| | | AGATCACCAAGCACGTGGCCCAGATCCTGGACAGCCGGATGAACA |
| | | CCAAGTACGACGAGAACGACAAGCTGATCCGGGAGGTGAAGGTGA |
| | | TCACCCTGAAGAGCAAGCTGGTGAGCGACTTCCGGAAGGACTTCCA |
| | | GTTCTACAAGGTGCGGGAGATCAACAACTACCACCACGCCCACGAC |
| | | GCCTACCTGAACGCCGTGGTGGGCACCGCCCTGATCAAGAAGTACC |
| | | CCAAGCTGGAGAGCGAGTTCGTGTACGGCGACTACAAGGTGTACGA |
| | | CGTGCGGAAGATGATCGCCAAGAGCGAGCAGGAGATCGGCAAGGC |
| | | CACCGCCAAGTACTTCTTCTACAGCAACATCATGAACTTCTTCAAGA |
| | | CCGAGATCACCCTGGCCAACGGCGAGATCCGGAAGCGGCCCCTGAT |
| | | CGAGACCAACGGCGAGACCGGCGAGATCGTGTGGGACAAGGGCCG |
| | | GGACTTCGCCACCGTGCGGAAGGTGCTGAGCATGCCCCAGGTGAAC |
| | | ATCGTGAAGAAGACCGAGGTGCAGACCGGCGGCTTCAGCAAGGAG |
| | | AGCATCCTGCCCAAGCGGAACAGCGACAAGCTGATCGCCCGGAAG |
| | | AAGGACTGGGACCCCAAGAAGTACGGCGGCTTCGACAGCCCCACC |
| | | GTGGCCTACAGCGTGCTGGTGGTGGCCAAGGTGGAGAAGGGCAAG |
| | | AGCAAGAAGCTGAAGAGCGTGAAGGAGCTGCTGGGCATCACCATC |
| | | ATGGAGCGGAGCAGCTTCGAGAAGAACCCCATCGACTTCCTGGAGG |
| | | CCAAGGGCTACAAGGAGGTGAAGAAGGACCTGATCATCAAGCTGC |
| | | CCAAGTACAGCCTGTTCGAGCTGGAGAACGGCCGGAAGCGGATGCT |
| | | GGCCAGCGCCGGCGAGCTGCAGAAGGGCAACGAGCTGGCCCTGCC |
| | | CAGCAAGTACGTGAACTTCCTGTACCTGGCCAGCCACTACGAGAAG |
| | | CTGAAGGGCAGCCCCGAGGACAACGAGCAGAAGCAGCTGTTCGTG |
| | | GAGCAGCACAAGCACTACCTGGACGAGATCATCGAGCAGATCAGC |
| | | GAGTTCAGCAAGCGGGTGATCCTGGCCGACGCCAACCTGGACAAG |
| | | GTGCTGAGCGCCTACAACAAGCACCGGGACAAGCCCATCCGGGAG |
| | | CAGGCCGAGAACATCATCCACCTGTTCACCCTGACCAACCTGGGCG |
| | | CCCCCGCCGCCTTCAAGTACTTCGACACCACCATCGACCGGAAGCG |
| | | GTACACCAGCACCAAGGAGGTGCTGGACGCCACCCTGATCCACCAG |
| | | AGCATCACCGGCCTGTACGAGACCCGGATCGACCTGAGCCAGCTGG |
| | | GCGGCGACGGCAGCGGCAGCCCCAAGAAGAAGCGGAAGGTGGACG |
| | | GCAGCCCCAAGAAGAAGCGGAAGGTGGACAGCGGCTGA |
| 52 | Cas9 nickase | ATGGACAAGAAGTACAGCATCGGCCTGGCCATCGGCACCAACAGC |
| | ORF using low | GTGGGCTGGGCCGTGATCACCGACGAGTACAAGGTGCCCAGCAAG |
| | A/U codons of | AAGTTCAAGGTGCTGGGCAACACCGACCGGCACAGCATCAAGAAG |
| | Table 4, with start | AACCTGATCGGCGCCCTGCTGTTCGACAGCGGCGAGACCGCCGAGG |
| | and stop codons | CCACCCGGCTGAAGCGGACCGCCCGGCGGCGGTACACCCGGCGGA |
| | and no NLS | AGAACCGGATCTGCTACCTGCAGGAGATCTTCAGCAACGAGATGGC |
| | | CAAGGTGGACGACAGCTTCTTCCACCGGCTGGAGGAGAGCTTCCTG |
| | | GTGGAGGAGGACAAGAAGCACGAGCGGCACCCCATCTTCGGCAAC |
| | | ATCGTGGACGAGGTGGCCTACCACGAGAAGTACCCCACCATCTACC |
| | | ACCTGCGGAAGAAGCTGGTGGACAGCACCGACAAGGCCGACCTGC |
| | | GGCTGATCTACCTGGCCCTGGCCCACATGATCAAGTTCCGGGGCCA |
| | | CTTCCTGATCGAGGGCGACCTGAACCCCGACAACAGCGACGTGGAC |
| | | AAGCTGTTCATCCAGCTGGTGCAGACCTACAACCAGCTGTTCGAGG |
| | | AGAACCCCATCAACGCCAGCGGCGTGGACGCCAAGGCCATCCTGA |
| | | GCGCCCGGCTGAGCAAGAGCCGGCGGCTGGAGAACCTGATCGCCC |
| | | AGCTGCCCGGCGAGAAGAAGAACGGCCTGTTCGGCAACCTGATCGC |
| | | CCTGAGCCTGGGCCTGACCCCCAACTTCAAGAGCAACTTCGACCTG |
| | | GCCGAGGACGCCAAGCTGCAGCTGAGCAAGGACACCTACGACGAC |
| | | GACCTGGACAACCTGCTGGCCCAGATCGGCGACCAGTACGCCGACC |
| | | TGTTCCTGGCCGCCAAGAACCTGAGCGACGCCATCCTGCTGAGCGA |
| | | CATCCTGCGGGTGAACACCGAGATCACCAAGGCCCCCCTGAGCGCC |
| | | AGCATGATCAAGCGGTACGACGAGCACCACCAGGACCTGACCCTGC |
| | | TGAAGGCCCTGGTGCGGCAGCAGCTGCCCGAGAAGTACAAGGAGA |
| | | TCTTCTTCGACCAGAGCAAGAACGGCTACGCCGGCTACATCGACGG |
| | | CGGCGCCAGCCAGGAGGAGTTCTACAAGTTCATCAAGCCCATCCTG |
| | | GAGAAGATGGACGGCACCGAGGAGCTGCTGGTGAAGCTGAACCGG |
| | | GAGGACCTGCTGCGGAAGCAGCGGACCTTCGACAACGGCAGCATC |
| | | CCCCACCAGATCCACCTGGGCGAGCTGCACGCCATCCTGCGGCGGC |
| | | AGGAGGACTTCTACCCCTTCCTGAAGGACAACCGGGAGAAGATCGA |
| | | GAAGATCCTGACCTTCCGGATCCCCTACTACGTGGGCCCCCTGGCC |
| | | CGGGGCAACAGCCGGTTCGCCTGGATGACCCGGAAGAGCGAGGAG |
| | | ACCATCACCCCCTGGAACTTCGAGGAGGTGGTGGACAAGGGCGCCA |
| | | GCGCCCAGAGCTTCATCGAGCGGATGACCAACTTCGACAAGAACCT |
| | | GCCCAACGAGAAGGTGCTGCCCAAGCACAGCCTGCTGTACGAGTAC |
| | | TTCACCGTGTACAACGAGCTGACCAAGGTGAAGTACGTGACCGAGG |
| | | GCATGCGGAAGCCCGCCTTCCTGAGCGGCGAGCAGAAGAAGGCCA |
| | | TCGTGGACCTGCTGTTCAAGACCAACCGGAAGGTGACCGTGAAGCA |
| | | GCTGAAGGAGGACTACTTCAAGAAGATCGAGTGCTTCGACAGCGTG |
| | | GAGATCAGCGGCGTGGAGGACCGGTTCAACGCCAGCCTGGGCACCT |
| | | ACCACGACCTGCTGAAGATCATCAAGGACAAGGACTTCCTGGACAA |
| | | CGAGGAGAACGAGGACATCCTGGAGGACATCGTGCTGACCCTGAC |
| | | CCTGTTCGAGGACCGGGAGATGATCGAGGAGCGGCTGAAGACCTA |
| | | CGCCCACCTGTTCGACGACAAGGTGATGAAGCAGCTGAAGCGGCG |
| | | GCGGTACACCGGCTGGGGCCGGCTGAGCCGGAAGCTGATCAACGG |
| | | CATCCGGGACAAGCAGAGCGGCAAGACCATCCTGGACTTCCTGAAG |
| | | AGCGACGGCTTCGCCAACCGGAACTTCATGCAGCTGATCCACGACG |
| | | ACAGCCTGACCTTCAAGGAGGACATCCAGAAGGCCCAGGTGAGCG |
| | | GCCAGGGCGACAGCCTGCACGAGCACATCGCCAACCTGGCCGGCA |
| | | GCCCCGCCATCAAGAAGGGCATCCTGCAGACCGTGAAGGTGGTGG |
| | | ACGAGCTGGTGAAGGTGATGGGCCGGCACAAGCCCGAGAACATCG |
| | | TGATCGAGATGGCCCGGGAGAACCAGACCACCCAGAAGGGCCAGA |
| | | AGAACAGCCGGGAGCGGATGAAGCGGATCGAGGAGGGCATCAAGG |
| | | AGCTGGGCAGCCAGATCCTGAAGGAGCACCCCGTGGAGAACACCC |
| | | AGCTGCAGAACGAGAAGCTGTACCTGTACTACCTGCAGAACGGCCG |
| | | GGACATGTACGTGGACCAGGAGCTGGACATCAACCGGCTGAGCGA |
| | | CTACGACGTGGACCACATCGTGCCCCAGAGCTTCCTGAAGGACGAC |
| | | AGCATCGACAACAAGGTGCTGACCCGGAGCGACAAGAACCGGGGC |
| | | AAGAGCGACAACGTGCCCAGCGAGGAGGTGGTGAAGAAGATGAAG |
| | | AACTACTGGCGGCAGCTGCTGAACGCCAAGCTGATCACCCAGCGGA |
| | | AGTTCGACAACCTGACCAAGGCCGAGCGGGGCGGCCTGAGCGAGC |
| | | TGGACAAGGCCGGCTTCATCAAGCGGCAGCTGGTGGAGACCCGGC |
| | | AGATCACCAAGCACGTGGCCCAGATCCTGGACAGCCGGATGAACA |
| | | CCAAGTACGACGAGAACGACAAGCTGATCCGGGAGGTGAAGGTGA |
| | | TCACCCTGAAGAGCAAGCTGGTGAGCGACTTCCGGAAGGACTTCCA |
| | | GTTCTACAAGGTGCGGGAGATCAACAACTACCACCACGCCCACGAC |
| | | GCCTACCTGAACGCCGTGGTGGGCACCGCCCTGATCAAGAAGTACC |
| | | CCAAGCTGGAGAGCGAGTTCGTGTACGGCGACTACAAGGTGTACGA |
| | | CGTGCGGAAGATGATCGCCAAGAGCGAGCAGGAGATCGGCAAGGC |
| | | CACCGCCAAGTACTTCTTCTACAGCAACATCATGAACTTCTTCAAGA |
| | | CCGAGATCACCCTGGCCAACGGCGAGATCCGGAAGCGGCCCCTGAT |
| | | CGAGACCAACGGCGAGACCGGCGAGATCGTGTGGGACAAGGGCCG |
| | | GGACTTCGCCACCGTGCGGAAGGTGCTGAGCATGCCCCAGGTGAAC |
| | | ATCGTGAAGAAGACCGAGGTGCAGACCGGCGGCTTCAGCAAGGAG |
| | | AGCATCCTGCCCAAGCGGAACAGCGACAAGCTGATCGCCCGGAAG |
| | | AAGGACTGGGACCCCAAGAAGTACGGCGGCTTCGACAGCCCCACC |
| | | GTGGCCTACAGCGTGCTGGTGGTGGCCAAGGTGGAGAAGGGCAAG |
| | | AGCAAGAAGCTGAAGAGCGTGAAGGAGCTGCTGGGCATCACCATC |
| | | ATGGAGCGGAGCAGCTTCGAGAAGAACCCCATCGACTTCCTGGAGG |
| | | CCAAGGGCTACAAGGAGGTGAAGAAGGACCTGATCATCAAGCTGC |
| | | CCAAGTACAGCCTGTTCGAGCTGGAGAACGGCCGGAAGCGGATGCT |
| | | GGCCAGCGCCGGCGAGCTGCAGAAGGGCAACGAGCTGGCCCTGCC |
| | | CAGCAAGTACGTGAACTTCCTGTACCTGGCCAGCCACTACGAGAAG |
| | | CTGAAGGGCAGCCCCGAGGACAACGAGCAGAAGCAGCTGTTCGTG |
| | | GAGCAGCACAAGCACTACCTGGACGAGATCATCGAGCAGATCAGC |
| | | GAGTTCAGCAAGCGGGTGATCCTGGCCGACGCCAACCTGGACAAG |
| | | GTGCTGAGCGCCTACAACAAGCACCGGGACAAGCCCATCCGGGAG |
| | | CAGGCCGAGAACATCATCCACCTGTTCACCCTGACCAACCTGGGCG |
| | | CCCCCGCCGCCTTCAAGTACTTCGACACCACCATCGACCGGAAGCG |
| | | GTACACCAGCACCAAGGAGGTGCTGGACGCCACCCTGATCCACCAG |
| | | AGCATCACCGGCCTGTACGAGACCCGGATCGACCTGAGCCAGCTGG |
| | | GCGGCGACTGA |
| 53 | Cas9 nickase | GACAAGAAGTACTCCATCGGCCTGGCCATCGGCACCAACTCCGTGG |
| | ORF using low A | GCTGGGCCGTGATCACCGACGAGTACAAGGTGCCCTCCAAGAAGTT |
| | codons of Table 4 | CAAGGTGCTGGGCAACACCGACCGGCACTCCATCAAGAAGAACCT |
| | (no start or stop | GATCGGCGCCCTGCTGTTCGACTCCGGCGAGACCGCCGAGGCCACC |
| | codons; suitable | CGGCTGAAGCGGACCGCCCGGCGGCGGTACACCCGGCGGAAGAAC |
| | for inclusion in | CGGATCTGCTACCTGCAGGAGATCTTCTCCAACGAGATGGCCAAGG |
| | fusion protein | TGGACGACTCCTTCTTCCACCGGCTGGAGGAGTCCTTCCTGGTGGA |
| | coding sequence) | GGAGGACAAGAAGCACGAGCGGCACCCCATCTTCGGCAACATCGT |
| | | GGACGAGGTGGCCTACCACGAGAAGTACCCCACCATCTACCACCTG |
| | | CGGAAGAAGCTGGTGGACTCCACCGACAAGGCCGACCTGCGGCTG |
| | | ATCTACCTGGCCCTGGCCCACATGATCAAGTTCCGGGGCCACTTCCT |
| | | GATCGAGGGCGACCTGAACCCCGACAACTCCGACGTGGACAAGCT |
| | | GTTCATCCAGCTGGTGCAGACCTACAACCAGCTGTTCGAGGAGAAC |
| | | CCCATCAACGCCTCCGGCGTGGACGCCAAGGCCATCCTGTCCGCCC |
| | | GGCTGTCCAAGTCCCGGCGGCTGGAGAACCTGATCGCCCAGCTGCC |
| | | CGGCGAGAAGAAGAACGGCCTGTTCGGCAACCTGATCGCCCTGTCC |
| | | CTGGGCCTGACCCCCAACTTCAAGTCCAACTTCGACCTGGCCGAGG |
| | | ACGCCAAGCTGCAGCTGTCCAAGGACACCTACGACGACGACCTGGA |
| | | CAACCTGCTGGCCCAGATCGGCGACCAGTACGCCGACCTGTTCCTG |
| | | GCCGCCAAGAACCTGTCCGACGCCATCCTGCTGTCCGACATCCTGC |
| | | GGGTGAACACCGAGATCACCAAGGCCCCCCTGTCCGCCTCCATGAT |
| | | CAAGCGGTACGACGAGCACCACCAGGACCTGACCCTGCTGAAGGC |
| | | CCTGGTGCGGCAGCAGCTGCCCGAGAAGTACAAGGAGATCTTCTTC |
| | | GACCAGTCCAAGAACGGCTACGCCGGCTACATCGACGGCGGCGCCT |
| | | CCCAGGAGGAGTTCTACAAGTTCATCAAGCCCATCCTGGAGAAGAT |
| | | GGACGGCACCGAGGAGCTGCTGGTGAAGCTGAACCGGGAGGACCT |
| | | GCTGCGGAAGCAGCGGACCTTCGACAACGGCTCCATCCCCCACCAG |
| | | ATCCACCTGGGCGAGCTGCACGCCATCCTGCGGCGGCAGGAGGACT |
| | | TCTACCCCTTCCTGAAGGACAACCGGGAGAAGATCGAGAAGATCCT |
| | | GACCTTCCGGATCCCCTACTACGTGGGCCCCCTGGCCCGGGGCAAC |
| | | TCCCGGTTCGCCTGGATGACCCGGAAGTCCGAGGAGACCATCACCC |
| | | CCTGGAACTTCGAGGAGGTGGTGGACAAGGGCGCCTCCGCCCAGTC |
| | | CTTCATCGAGCGGATGACCAACTTCGACAAGAACCTGCCCAACGAG |
| | | AAGGTGCTGCCCAAGCACTCCCTGCTGTACGAGTACTTCACCGTGT |
| | | ACAACGAGCTGACCAAGGTGAAGTACGTGACCGAGGGCATGCGGA |
| | | AGCCCGCCTTCCTGTCCGGCGAGCAGAAGAAGGCCATCGTGGACCT |
| | | GCTGTTCAAGACCAACCGGAAGGTGACCGTGAAGCAGCTGAAGGA |
| | | GGACTACTTCAAGAAGATCGAGTGCTTCGACTCCGTGGAGATCTCC |
| | | GGCGTGGAGGACCGGTTCAACGCCTCCCTGGGCACCTACCACGACC |
| | | TGCTGAAGATCATCAAGGACAAGGACTTCCTGGACAACGAGGAGA |
| | | ACGAGGACATCCTGGAGGACATCGTGCTGACCCTGACCCTGTTCGA |
| | | GGACCGGGAGATGATCGAGGAGCGGCTGAAGACCTACGCCCACCT |
| | | GTTCGACGACAAGGTGATGAAGCAGCTGAAGCGGCGGCGGTACAC |
| | | CGGCTGGGGCCGGCTGTCCCGGAAGCTGATCAACGGCATCCGGGAC |
| | | AAGCAGTCCGGCAAGACCATCCTGGACTTCCTGAAGTCCGACGGCT |
| | | TCGCCAACCGGAACTTCATGCAGCTGATCCACGACGACTCCCTGAC |
| | | CTTCAAGGAGGACATCCAGAAGGCCCAGGTGTCCGGCCAGGGCGA |
| | | CTCCCTGCACGAGCACATCGCCAACCTGGCCGGCTCCCCCGCCATC |
| | | AAGAAGGGCATCCTGCAGACCGTGAAGGTGGTGGACGAGCTGGTG |
| | | AAGGTGATGGGCCGGCACAAGCCCGAGAACATCGTGATCGAGATG |
| | | GCCCGGGAGAACCAGACCACCCAGAAGGGCCAGAAGAACTCCCGG |
| | | GAGCGGATGAAGCGGATCGAGGAGGGCATCAAGGAGCTGGGCTCC |
| | | CAGATCCTGAAGGAGCACCCCGTGGAGAACACCCAGCTGCAGAAC |
| | | GAGAAGCTGTACCTGTACTACCTGCAGAACGGCCGGGACATGTACG |
| | | TGGACCAGGAGCTGGACATCAACCGGCTGTCCGACTACGACGTGGA |
| | | CCACATCGTGCCCCAGTCCTTCCTGAAGGACGACTCCATCGACAAC |
| | | AAGGTGCTGACCCGGTCCGACAAGAACCGGGGCAAGTCCGACAAC |
| | | GTGCCCTCCGAGGAGGTGGTGAAGAAGATGAAGAACTACTGGCGG |
| | | CAGCTGCTGAACGCCAAGCTGATCACCCAGCGGAAGTTCGACAACC |
| | | TGACCAAGGCCGAGCGGGGCGGCCTGTCCGAGCTGGACAAGGCCG |
| | | GCTTCATCAAGCGGCAGCTGGTGGAGACCCGGCAGATCACCAAGCA |
| | | CGTGGCCCAGATCCTGGACTCCCGGATGAACACCAAGTACGACGAG |
| | | AACGACAAGCTGATCCGGGAGGTGAAGGTGATCACCCTGAAGTCC |
| | | AAGCTGGTGTCCGACTTCCGGAAGGACTTCCAGTTCTACAAGGTGC |
| | | GGGAGATCAACAACTACCACCACGCCCACGACGCCTACCTGAACGC |
| | | CGTGGTGGGCACCGCCCTGATCAAGAAGTACCCCAAGCTGGAGTCC |
| | | GAGTTCGTGTACGGCGACTACAAGGTGTACGACGTGCGGAAGATGA |
| | | TCGCCAAGTCCGAGCAGGAGATCGGCAAGGCCACCGCCAAGTACTT |
| | | CTTCTACTCCAACATCATGAACTTCTTCAAGACCGAGATCACCCTGG |
| | | CCAACGGCGAGATCCGGAAGCGGCCCCTGATCGAGACCAACGGCG |
| | | AGACCGGCGAGATCGTGTGGGACAAGGGCCGGGACTTCGCCACCG |
| | | TGCGGAAGGTGCTGTCCATGCCCCAGGTGAACATCGTGAAGAAGAC |
| | | CGAGGTGCAGACCGGCGGCTTCTCCAAGGAGTCCATCCTGCCCAAG |
| | | CGGAACTCCGACAAGCTGATCGCCCGGAAGAAGGACTGGGACCCC |
| | | AAGAAGTACGGCGGCTTCGACTCCCCCACCGTGGCCTACTCCGTGC |
| | | TGGTGGTGGCCAAGGTGGAGAAGGGCAAGTCCAAGAAGCTGAAGT |
| | | CCGTGAAGGAGCTGCTGGGCATCACCATCATGGAGCGGTCCTCCTT |
| | | CGAGAAGAACCCCATCGACTTCCTGGAGGCCAAGGGCTACAAGGA |
| | | GGTGAAGAAGGACCTGATCATCAAGCTGCCCAAGTACTCCCTGTTC |
| | | GAGCTGGAGAACGGCCGGAAGCGGATGCTGGCCTCCGCCGGCGAG |
| | | CTGCAGAAGGGCAACGAGCTGGCCCTGCCCTCCAAGTACGTGAACT |
| | | TCCTGTACCTGGCCTCCCACTACGAGAAGCTGAAGGGCTCCCCCGA |
| | | GGACAACGAGCAGAAGCAGCTGTTCGTGGAGCAGCACAAGCACTA |
| | | CCTGGACGAGATCATCGAGCAGATCTCCGAGTTCTCCAAGCGGGTG |
| | | ATCCTGGCCGACGCCAACCTGGACAAGGTGCTGTCCGCCTACAACA |
| | | AGCACCGGGACAAGCCCATCCGGGAGCAGGCCGAGAACATCATCC |
| | | ACCTGTTCACCCTGACCAACCTGGGCGCCCCCGCCGCCTTCAAGTA |
| | | CTTCGACACCACCATCGACCGGAAGCGGTACACCTCCACCAAGGAG |
| | | GTGCTGGACGCCACCCTGATCCACCAGTCCATCACCGGCCTGTACG |
| | | AGACCCGGATCGACCTGTCCCAGCTGGGCGGCGACGGCGGCGGCTC |
| | | CCCCAAGAAGAAGCGGAAGGTG |
| 54 | Cas9 nickase | GACAAGAAGTACTCCATCGGCCTGGCCATCGGCACCAACTCCGTGG |
| | ORF using low A | GCTGGGCCGTGATCACCGACGAGTACAAGGTGCCCTCCAAGAAGTT |
| | codons of Table 4 | CAAGGTGCTGGGCAACACCGACCGGCACTCCATCAAGAAGAACCT |
| | (no NLS and no | GATCGGCGCCCTGCTGTTCGACTCCGGCGAGACCGCCGAGGCCACC |
| | start or stop | CGGCTGAAGCGGACCGCCCGGCGGCGGTACACCCGGCGGAAGAAC |
| | codons; suitable | CGGATCTGCTACCTGCAGGAGATCTTCTCCAACGAGATGGCCAAGG |
| | for inclusion in | TGGACGACTCCTTCTTCCACCGGCTGGAGGAGTCCTTCCTGGTGGA |
| | fusion protein | GGAGGACAAGAAGCACGAGCGGCACCCCATCTTCGGCAACATCGT |
| | coding sequence) | GGACGAGGTGGCCTACCACGAGAAGTACCCCACCATCTACCACCTG |
| | | CGGAAGAAGCTGGTGGACTCCACCGACAAGGCCGACCTGCGGCTG |
| | | ATCTACCTGGCCCTGGCCCACATGATCAAGTTCCGGGGCCACTTCCT |
| | | GATCGAGGGCGACCTGAACCCCGACAACTCCGACGTGGACAAGCT |
| | | GTTCATCCAGCTGGTGCAGACCTACAACCAGCTGTTCGAGGAGAAC |
| | | CCCATCAACGCCTCCGGCGTGGACGCCAAGGCCATCCTGTCCGCCC |
| | | GGCTGTCCAAGTCCCGGCGGCTGGAGAACCTGATCGCCCAGCTGCC |
| | | CGGCGAGAAGAAGAACGGCCTGTTCGGCAACCTGATCGCCCTGTCC |
| | | CTGGGCCTGACCCCCAACTTCAAGTCCAACTTCGACCTGGCCGAGG |
| | | ACGCCAAGCTGCAGCTGTCCAAGGACACCTACGACGACGACCTGGA |
| | | CAACCTGCTGGCCCAGATCGGCGACCAGTACGCCGACCTGTTCCTG |
| | | GCCGCCAAGAACCTGTCCGACGCCATCCTGCTGTCCGACATCCTGC |
| | | GGGTGAACACCGAGATCACCAAGGCCCCCCTGTCCGCCTCCATGAT |
| | | CAAGCGGTACGACGAGCACCACCAGGACCTGACCCTGCTGAAGGC |
| | | CCTGGTGCGGCAGCAGCTGCCCGAGAAGTACAAGGAGATCTTCTTC |
| | | GACCAGTCCAAGAACGGCTACGCCGGCTACATCGACGGCGGCGCCT |
| | | CCCAGGAGGAGTTCTACAAGTTCATCAAGCCCATCCTGGAGAAGAT |
| | | GGACGGCACCGAGGAGCTGCTGGTGAAGCTGAACCGGGAGGACCT |
| | | GCTGCGGAAGCAGCGGACCTTCGACAACGGCTCCATCCCCCACCAG |
| | | ATCCACCTGGGCGAGCTGCACGCCATCCTGCGGCGGCAGGAGGACT |
| | | TCTACCCCTTCCTGAAGGACAACCGGGAGAAGATCGAGAAGATCCT |
| | | GACCTTCCGGATCCCCTACTACGTGGGCCCCCTGGCCCGGGGCAAC |
| | | TCCCGGTTCGCCTGGATGACCCGGAAGTCCGAGGAGACCATCACCC |
| | | CCTGGAACTTCGAGGAGGTGGTGGACAAGGGCGCCTCCGCCCAGTC |
| | | CTTCATCGAGCGGATGACCAACTTCGACAAGAACCTGCCCAACGAG |
| | | AAGGTGCTGCCCAAGCACTCCCTGCTGTACGAGTACTTCACCGTGT |
| | | ACAACGAGCTGACCAAGGTGAAGTACGTGACCGAGGGCATGCGGA |
| | | AGCCCGCCTTCCTGTCCGGCGAGCAGAAGAAGGCCATCGTGGACCT |
| | | GCTGTTCAAGACCAACCGGAAGGTGACCGTGAAGCAGCTGAAGGA |
| | | GGACTACTTCAAGAAGATCGAGTGCTTCGACTCCGTGGAGATCTCC |
| | | GGCGTGGAGGACCGGTTCAACGCCTCCCTGGGCACCTACCACGACC |
| | | TGCTGAAGATCATCAAGGACAAGGACTTCCTGGACAACGAGGAGA |
| | | ACGAGGACATCCTGGAGGACATCGTGCTGACCCTGACCCTGTTCGA |
| | | GGACCGGGAGATGATCGAGGAGCGGCTGAAGACCTACGCCCACCT |
| | | GTTCGACGACAAGGTGATGAAGCAGCTGAAGCGGCGGCGGTACAC |
| | | CGGCTGGGGCCGGCTGTCCCGGAAGCTGATCAACGGCATCCGGGAC |
| | | AAGCAGTCCGGCAAGACCATCCTGGACTTCCTGAAGTCCGACGGCT |
| | | TCGCCAACCGGAACTTCATGCAGCTGATCCACGACGACTCCCTGAC |
| | | CTTCAAGGAGGACATCCAGAAGGCCCAGGTGTCCGGCCAGGGCGA |
| | | CTCCCTGCACGAGCACATCGCCAACCTGGCCGGCTCCCCCGCCATC |
| | | AAGAAGGGCATCCTGCAGACCGTGAAGGTGGTGGACGAGCTGGTG |
| | | AAGGTGATGGGCCGGCACAAGCCCGAGAACATCGTGATCGAGATG |
| | | GCCCGGGAGAACCAGACCACCCAGAAGGGCCAGAAGAACTCCCGG |
| | | GAGCGGATGAAGCGGATCGAGGAGGGCATCAAGGAGCTGGGCTCC |
| | | CAGATCCTGAAGGAGCACCCCGTGGAGAACACCCAGCTGCAGAAC |
| | | GAGAAGCTGTACCTGTACTACCTGCAGAACGGCCGGGACATGTACG |
| | | TGGACCAGGAGCTGGACATCAACCGGCTGTCCGACTACGACGTGGA |
| | | CCACATCGTGCCCCAGTCCTTCCTGAAGGACGACTCCATCGACAAC |
| | | AAGGTGCTGACCCGGTCCGACAAGAACCGGGGCAAGTCCGACAAC |
| | | GTGCCCTCCGAGGAGGTGGTGAAGAAGATGAAGAACTACTGGCGG |
| | | CAGCTGCTGAACGCCAAGCTGATCACCCAGCGGAAGTTCGACAACC |
| | | TGACCAAGGCCGAGCGGGGCGGCCTGTCCGAGCTGGACAAGGCCG |
| | | GCTTCATCAAGCGGCAGCTGGTGGAGACCCGGCAGATCACCAAGCA |
| | | CGTGGCCCAGATCCTGGACTCCCGGATGAACACCAAGTACGACGAG |
| | | AACGACAAGCTGATCCGGGAGGTGAAGGTGATCACCCTGAAGTCC |
| | | AAGCTGGTGTCCGACTTCCGGAAGGACTTCCAGTTCTACAAGGTGC |
| | | GGGAGATCAACAACTACCACCACGCCCACGACGCCTACCTGAACGC |
| | | CGTGGTGGGCACCGCCCTGATCAAGAAGTACCCCAAGCTGGAGTCC |
| | | GAGTTCGTGTACGGCGACTACAAGGTGTACGACGTGCGGAAGATGA |
| | | TCGCCAAGTCCGAGCAGGAGATCGGCAAGGCCACCGCCAAGTACTT |
| | | CTTCTACTCCAACATCATGAACTTCTTCAAGACCGAGATCACCCTGG |
| | | CCAACGGCGAGATCCGGAAGCGGCCCCTGATCGAGACCAACGGCG |
| | | AGACCGGCGAGATCGTGTGGGACAAGGGCCGGGACTTCGCCACCG |
| | | TGCGGAAGGTGCTGTCCATGCCCCAGGTGAACATCGTGAAGAAGAC |
| | | CGAGGTGCAGACCGGCGGCTTCTCCAAGGAGTCCATCCTGCCCAAG |
| | | CGGAACTCCGACAAGCTGATCGCCCGGAAGAAGGACTGGGACCCC |
| | | AAGAAGTACGGCGGCTTCGACTCCCCCACCGTGGCCTACTCCGTGC |
| | | TGGTGGTGGCCAAGGTGGAGAAGGGCAAGTCCAAGAAGCTGAAGT |
| | | CCGTGAAGGAGCTGCTGGGCATCACCATCATGGAGCGGTCCTCCTT |
| | | CGAGAAGAACCCCATCGACTTCCTGGAGGCCAAGGGCTACAAGGA |
| | | GGTGAAGAAGGACCTGATCATCAAGCTGCCCAAGTACTCCCTGTTC |
| | | GAGCTGGAGAACGGCCGGAAGCGGATGCTGGCCTCCGCCGGCGAG |
| | | CTGCAGAAGGGCAACGAGCTGGCCCTGCCCTCCAAGTACGTGAACT |
| | | TCCTGTACCTGGCCTCCCACTACGAGAAGCTGAAGGGCTCCCCCGA |
| | | GGACAACGAGCAGAAGCAGCTGTTCGTGGAGCAGCACAAGCACTA |
| | | CCTGGACGAGATCATCGAGCAGATCTCCGAGTTCTCCAAGCGGGTG |
| | | ATCCTGGCCGACGCCAACCTGGACAAGGTGCTGTCCGCCTACAACA |
| | | AGCACCGGGACAAGCCCATCCGGGAGCAGGCCGAGAACATCATCC |
| | | ACCTGTTCACCCTGACCAACCTGGGCGCCCCCGCCGCCTTCAAGTA |
| | | CTTCGACACCACCATCGACCGGAAGCGGTACACCTCCACCAAGGAG |
| | | GTGCTGGACGCCACCCTGATCCACCAGTCCATCACCGGCCTGTACG |
| | | AGACCCGGATCGACCTGTCCCAGCTGGGCGGCGAC |
| 55 | Cas9 nickase | GACAAGAAGTACTCCATCGGCCTGGCCATCGGCACCAACTCCGTGG |
| | ORF using low A | GCTGGGCCGTGATCACCGACGAGTACAAGGTGCCCTCCAAGAAGTT |
| | codons of Table | CAAGGTGCTGGGCAACACCGACCGGCACTCCATCAAGAAGAACCT |
| | 4, with two C- | GATCGGCGCCCTGCTGTTCGACTCCGGCGAGACCGCCGAGGCCACC |
| | terminal NLS | CGGCTGAAGCGGACCGCCCGGCGGCGGTACACCCGGCGGAAGAAC |
| | sequences (no | CGGATCTGCTACCTGCAGGAGATCTTCTCCAACGAGATGGCCAAGG |
| | start or stop | TGGACGACTCCTTCTTCCACCGGCTGGAGGAGTCCTTCCTGGTGGA |
| | codons; suitable | GGAGGACAAGAAGCACGAGCGGCACCCCATCTTCGGCAACATCGT |
| | for inclusion in | GGACGAGGTGGCCTAC |
| | fusion protein | CACGAGAAGTACCCCACCATCTACCACCTGCGGAAGAAGCTGGTGG |
| | coding sequence) | ACTCCACCGACAAGGCCGACCTGCGGCTGATCTACCTGGCCCTGGC |
| | | CCACATGATCAAGTTCCGGGGCCACTTCCTGATCGAGGGCGACCTG |
| | | AACCCCGACAACTCCGACGTGGACAAGCTGTTCATCCAGCTGGTGC |
| | | AGACCTACAACCAGCTGTTCGAGGAGAACCCCATCAACGCCTCCGG |
| | | CGTGGACGCCAAGGCCATCCTGTCCGCCCGGCTGTCCAAGTCCCGG |
| | | CGGCTGGAGAACCTGATCGCCCAGCTGCCCGGCGAGAAGAAGAAC |
| | | GGCCTGTTCGGCAACCTGATCGCCCTGTCCCTGGGCCTGACCCCCA |
| | | ACTTCAAGTCCAACTTCGACCTGGCCGAGGACGCCAAGCTGCAGCT |
| | | GTCCAAGGACACCTACGACGACGACCTGGACAACCTGCTGGCCCAG |
| | | ATCGGCGACCAGTACGCCGACCTGTTCCTGGCCGCCAAGAACCTGT |
| | | CCGACGCCATCCTGCTGTCCGACATCCTGCGGGTGAACACCGAGAT |
| | | CACCAAGGCCCCCCTGTCCGCCTCCATGATCAAGCGGTACGACGAG |
| | | CACCACCAGGACCTGACCCTGCTGAAGGCCCTGGTGCGGCAGCAGC |
| | | TGCCCGAGAAGTACAAGGAGATCTTCTTCGACCAGTCCAAGAACGG |
| | | CTACGCCGGCTACATCGACGGCGGCGCCTCCCAGGAGGAGTTCTAC |
| | | AAGTTCATCAAGCCCATCCTGGAGAAGATGGACGGCACCGAGGAG |
| | | CTGCTGGTGAAGCTGAACCGGGAGGACCTGCTGCGGAAGCAGCGG |
| | | ACCTTCGACAACGGCTCCATCCCCCACCAGATCCACCTGGGCGAGC |
| | | TGCACGCCATCCTGCGGCGGCAGGAGGACTTCTACCCCTTCCTGAA |
| | | GGACAACCGGGAGAAGATCGAGAAGATCCTGACCTTCCGGATCCCC |
| | | TACTACGTGGGCCCCCTGGCCCGGGGCAACTCCCGGTTCGCCTGGA |
| | | TGACCCGGAAGTCCGAGGAGACCATCACCCCCTGGAACTTCGAGGA |
| | | GGTGGTGGACAAGGGCGCCTCCGCCCAGTCCTTCATCGAGCGGATG |
| | | ACCAACTTCGACAAGAACCTGCCCAACGAGAAGGTGCTGCCCAAGC |
| | | ACTCCCTGCTGTACGAGTACTTCACCGTGTACAACGAGCTGACCAA |
| | | GGTGAAGTACGTGACCGAGGGCATGCGGAAGCCCGCCTTCCTGTCC |
| | | GGCGAGCAGAAGAAGGCCATCGTGGACCTGCTGTTCAAGACCAAC |
| | | CGGAAGGTGACCGTGAAGCAGCTGAAGGAGGACTACTTCAAGAAG |
| | | ATCGAGTGCTTCGACTCCGTGGAGATCTCCGGCGTGGAGGACCGGT |
| | | TCAACGCCTCCCTGGGCACCTACCACGACCTGCTGAAGATCATCAA |
| | | GGACAAGGACTTCCTGGACAACGAGGAGAACGAGGACATCCTGGA |
| | | GGACATCGTGCTGACCCTGACCCTGTTCGAGGACCGGGAGATGATC |
| | | GAGGAGCGGCTGAAGACCTACGCCCACCTGTTCGACGACAAGGTG |
| | | ATGAAGCAGCTGAAGCGGCGGCGGTACACCGGCTGGGGCCGGCTG |
| | | TCCCGGAAGCTGATCAACGGCATCCGGGACAAGCAGTCCGGCAAG |
| | | ACCATCCTGGACTTCCTGAAGTCCGACGGCTTCGCCAACCGGAACT |
| | | TCATGCAGCTGATCCACGACGACTCCCTGACCTTCAAGGAGGACAT |
| | | CCAGAAGGCCCAGGTGTCCGGCCAGGGCGACTCCCTGCACGAGCAC |
| | | ATCGCCAACCTGGCCGGCTCCCCCGCCATCAAGAAGGGCATCCTGC |
| | | AGACCGTGAAGGTGGTGGACGAGCTGGTGAAGGTGATGGGCCGGC |
| | | ACAAGCCCGAGAACATCGTGATCGAGATGGCCCGGGAGAACCAGA |
| | | CCACCCAGAAGGGCCAGAAGAACTCCCGGGAGCGGATGAAGCGGA |
| | | TCGAGGAGGGCATCAAGGAGCTGGGCTCCCAGATCCTGAAGGAGC |
| | | ACCCCGTGGAGAACACCCAGCTGCAGAACGAGAAGCTGTACCTGTA |
| | | CTACCTGCAGAACGGCCGGGACATGTACGTGGACCAGGAGCTGGA |
| | | CATCAACCGGCTGTCCGACTACGACGTGGACCACATCGTGCCCCAG |
| | | TCCTTCCTGAAGGACGACTCCATCGACAACAAGGTGCTGACCCGGT |
| | | CCGACAAGAACCGGGGCAAGTCCGACAACGTGCCCTCCGAGGAGG |
| | | TGGTGAAGAAGATGAAGAACTACTGGCGGCAGCTGCTGAACGCCA |
| | | AGCTGATCACCCAGCGGAAGTTCGACAACCTGACCAAGGCCGAGC |
| | | GGGGCGGCCTGTCCGAGCTGGACAAGGCCGGCTTCATCAAGCGGCA |
| | | GCTGGTGGAGACCCGGCAGATCACCAAGCACGTGGCCCAGATCCTG |
| | | GACTCCCGGATGAACACCAAGTACGACGAGAACGACAAGCTGATC |
| | | CGGGAGGTGAAGGTGATCACCCTGAAGTCCAAGCTGGTGTCCGACT |
| | | TCCGGAAGGACTTCCAGTTCTACAAGGTGCGGGAGATCAACAACTA |
| | | CCACCACGCCCACGACGCCTACCTGAACGCCGTGGTGGGCACCGCC |
| | | CTGATCAAGAAGTACCCCAAGCTGGAGTCCGAGTTCGTGTACGGCG |
| | | ACTACAAGGTGTACGACGTGCGGAAGATGATCGCCAAGTCCGAGC |
| | | AGGAGATCGGCAAGGCCACCGCCAAGTACTTCTTCTACTCCAACAT |
| | | CATGAACTTCTTCAAGACCGAGATCACCCTGGCCAACGGCGAGATC |
| | | CGGAAGCGGCCCCTGATCGAGACCAACGGCGAGACCGGCGAGATC |
| | | GTGTGGGACAAGGGCCGGGACTTCGCCACCGTGCGGAAGGTGCTGT |
| | | CCATGCCCCAGGTGAACATCGTGAAGAAGACCGAGGTGCAGACCG |
| | | GCGGCTTCTCCAAGGAGTCCATCCTGCCCAAGCGGAACTCCGACAA |
| | | GCTGATCGCCCGGAAGAAGGACTGGGACCCCAAGAAGTACGGCGG |
| | | CTTCGACTCCCCCACCGTGGCCTACTCCGTGCTGGTGGTGGCCAAG |
| | | GTGGAGAAGGGCAAGTCCAAGAAGCTGAAGTCCGTGAAGGAGCTG |
| | | CTGGGCATCACCATCATGGAGCGGTCCTCCTTCGAGAAGAACCCCA |
| | | TCGACTTCCTGGAGGCCAAGGGCTACAAGGAGGTGAAGAAGGACC |
| | | TGATCATCAAGCTGCCCAAGTACTCCCTGTTCGAGCTGGAGAACGG |
| | | CCGGAAGCGGATGCTGGCCTCCGCCGGCGAGCTGCAGAAGGGCAA |
| | | CGAGCTGGCCCTGCCCTCCAAGTACGTGAACTTCCTGTACCTGGCCT |
| | | CCCACTACGAGAAGCTGAAGGGCTCCCCCGAGGACAACGAGCAGA |
| | | AGCAGCTGTTCGTGGAGCAGCACAAGCACTACCTGGACGAGATCAT |
| | | CGAGCAGATCTCCGAGTTCTCCAAGCGGGTGATCCTGGCCGACGCC |
| | | AACCTGGACAAGGTGCTGTCCGCCTACAACAAGCACCGGGACAAG |
| | | CCCATCCGGGAGCAGGCCGAGAACATCATCCACCTGTTCACCCTGA |
| | | CCAACCTGGGCGCCCCCGCCGCCTTCAAGTACTTCGACACCACCAT |
| | | CGACCGGAAGCGGTACACCTCCACCAAGGAGGTGCTGGACGCCAC |
| | | CCTGATCCACCAGTCCATCACCGGCCTGTACGAGACCCGGATCGAC |
| | | CTGTCCCAGCTGGGCGGCGACGGCTCCGGCTCCCCCAAGAAGAAGC |
| | | GGAAGGTGGACGGCTCCCCCAAGAAGAAGCGGAAGGTGGACTCCG |
| | | GC |
| 56 | Cas9 nickase | GACAAGAAGTACAGCATCGGCCTGGCCATCGGCACCAACAGCGTG |
| | ORF using low | GGCTGGGCCGTGATCACCGACGAGTACAAGGTGCCCAGCAAGAAG |
| | A/U codons of | TTCAAGGTGCTGGGCAACACCGACCGGCACAGCATCAAGAAGAAC |
| | Table 4 (no start | CTGATCGGCGCCCTGCTGTTCGACAGCGGCGAGACCGCCGAGGCCA |
| | or stop codons; | CCCGGCTGAAGCGGACCGCCCGGCGGCGGTACACCCGGCGGAAGA |
| | suitable for | ACCGGATCTGCTACCTGCAGGAGATCTTCAGCAACGAGATGGCCAA |
| | inclusion in | GGTGGACGACAGCTTCTTCCACCGGCTGGAGGAGAGCTTCCTGGTG |
| | fusion protein | GAGGAGGACAAGAAGCACGAGCGGCACCCCATCTTCGGCAACATC |
| | coding sequence) | GTGGACGAGGTGGCCTACCACGAGAAGTACCCCACCATCTACCACC |
| | | TGCGGAAGAAGCTGGTGGACAGCACCGACAAGGCCGACCTGCGGC |
| | | TGATCTACCTGGCCCTGGCCCACATGATCAAGTTCCGGGGCCACTTC |
| | | CTGATCGAGGGCGACCTGAACCCCGACAACAGCGACGTGGACAAG |
| | | CTGTTCATCCAGCTGGTGCAGACCTACAACCAGCTGTTCGAGGAGA |
| | | ACCCCATCAACGCCAGCGGCGTGGACGCCAAGGCCATCCTGAGCGC |
| | | CCGGCTGAGCAAGAGCCGGCGGCTGGAGAACCTGATCGCCCAGCT |
| | | GCCCGGCGAGAAGAAGAACGGCCTGTTCGGCAACCTGATCGCCCTG |
| | | AGCCTGGGCCTGACCCCCAACTTCAAGAGCAACTTCGACCTGGCCG |
| | | AGGACGCCAAGCTGCAGCTGAGCAAGGACACCTACGACGACGACC |
| | | TGGACAACCTGCTGGCCCAGATCGGCGACCAGTACGCCGACCTGTT |
| | | CCTGGCCGCCAAGAACCTGAGCGACGCCATCCTGCTGAGCGACATC |
| | | CTGCGGGTGAACACCGAGATCACCAAGGCCCCCCTGAGCGCCAGCA |
| | | TGATCAAGCGGTACGACGAGCACCACCAGGACCTGACCCTGCTGAA |
| | | GGCCCTGGTGCGGCAGCAGCTGCCCGAGAAGTACAAGGAGATCTTC |
| | | TTCGACCAGAGCAAGAACGGCTACGCCGGCTACATCGACGGCGGC |
| | | GCCAGCCAGGAGGAGTTCTACAAGTTCATCAAGCCCATCCTGGAGA |
| | | AGATGGACGGCACCGAGGAGCTGCTGGTGAAGCTGAACCGGGAGG |
| | | ACCTGCTGCGGAAGCAGCGGACCTTCGACAACGGCAGCATCCCCCA |
| | | CCAGATCCACCTGGGCGAGCTGCACGCCATCCTGCGGCGGCAGGAG |
| | | GACTTCTACCCCTTCCTGAAGGACAACCGGGAGAAGATCGAGAAGA |
| | | TCCTGACCTTCCGGATCCCCTACTACGTGGGCCCCCTGGCCCGGGGC |
| | | AACAGCCGGTTCGCCTGGATGACCCGGAAGAGCGAGGAGACCATC |
| | | ACCCCCTGGAACTTCGAGGAGGTGGTGGACAAGGGCGCCAGCGCC |
| | | CAGAGCTTCATCGAGCGGATGACCAACTTCGACAAGAACCTGCCCA |
| | | ACGAGAAGGTGCTGCCCAAGCACAGCCTGCTGTACGAGTACTTCAC |
| | | CGTGTACAACGAGCTGACCAAGGTGAAGTACGTGACCGAGGGCAT |
| | | GCGGAAGCCCGCCTTCCTGAGCGGCGAGCAGAAGAAGGCCATCGT |
| | | GGACCTGCTGTTCAAGACCAACCGGAAGGTGACCGTGAAGCAGCTG |
| | | AAGGAGGACTACTTCAAGAAGATCGAGTGCTTCGACAGCGTGGAG |
| | | ATCAGCGGCGTGGAGGACCGGTTCAACGCCAGCCTGGGCACCTACC |
| | | ACGACCTGCTGAAGATCATCAAGGACAAGGACTTCCTGGACAACGA |
| | | GGAGAACGAGGACATCCTGGAGGACATCGTGCTGACCCTGACCCTG |
| | | TTCGAGGACCGGGAGATGATCGAGGAGCGGCTGAAGACCTACGCC |
| | | CACCTGTTCGACGACAAGGTGATGAAGCAGCTGAAGCGGCGGCGG |
| | | TACACCGGCTGGGGCCGGCTGAGCCGGAAGCTGATCAACGGCATCC |
| | | GGGACAAGCAGAGCGGCAAGACCATCCTGGACTTCCTGAAGAGCG |
| | | ACGGCTTCGCCAACCGGAACTTCATGCAGCTGATCCACGACGACAG |
| | | CCTGACCTTCAAGGAGGACATCCAGAAGGCCCAGGTGAGCGGCCA |
| | | GGGCGACAGCCTGCACGAGCACATCGCCAACCTGGCCGGCAGCCCC |
| | | GCCATCAAGAAGGGCATCCTGCAGACCGTGAAGGTGGTGGACGAG |
| | | CTGGTGAAGGTGATGGGCCGGCACAAGCCCGAGAACATCGTGATC |
| | | GAGATGGCCCGGGAGAACCAGACCACCCAGAAGGGCCAGAAGAAC |
| | | AGCCGGGAGCGGATGAAGCGGATCGAGGAGGGCATCAAGGAGCTG |
| | | GGCAGCCAGATCCTGAAGGAGCACCCCGTGGAGAACACCCAGCTG |
| | | CAGAACGAGAAGCTGTACCTGTACTACCTGCAGAACGGCCGGGAC |
| | | ATGTACGTGGACCAGGAGCTGGACATCAACCGGCTGAGCGACTACG |
| | | ACGTGGACCACATCGTGCCCCAGAGCTTCCTGAAGGACGACAGCAT |
| | | CGACAACAAGGTGCTGACCCGGAGCGACAAGAACCGGGGCAAGAG |
| | | CGACAACGTGCCCAGCGAGGAGGTGGTGAAGAAGATGAAGAACTA |
| | | CTGGCGGCAGCTGCTGAACGCCAAGCTGATCACCCAGCGGAAGTTC |
| | | GACAACCTGACCAAGGCCGAGCGGGGCGGCCTGAGCGAGCTGGAC |
| | | AAGGCCGGCTTCATCAAGCGGCAGCTGGTGGAGACCCGGCAGATC |
| | | ACCAAGCACGTGGCCCAGATCCTGGACAGCCGGATGAACACCAAG |
| | | TACGACGAGAACGACAAGCTGATCCGGGAGGTGAAGGTGATCACC |
| | | CTGAAGAGCAAGCTGGTGAGCGACTTCCGGAAGGACTTCCAGTTCT |
| | | ACAAGGTGCGGGAGATCAACAACTACCACCACGCCCACGACGCCT |
| | | ACCTGAACGCCGTGGTGGGCACCGCCCTGATCAAGAAGTACCCCAA |
| | | GCTGGAGAGCGAGTTCGTGTACGGCGACTACAAGGTGTACGACGTG |
| | | CGGAAGATGATCGCCAAGAGCGAGCAGGAGATCGGCAAGGCCACC |
| | | GCCAAGTACTTCTTCTACAGCAACATCATGAACTTCTTCAAGACCG |
| | | AGATCACCCTGGCCAACGGCGAGATCCGGAAGCGGCCCCTGATCGA |
| | | GACCAACGGCGAGACCGGCGAGATCGTGTGGGACAAGGGCCGGGA |
| | | CTTCGCCACCGTGCGGAAGGTGCTGAGCATGCCCCAGGTGAACATC |
| | | GTGAAGAAGACCGAGGTGCAGACCGGCGGCTTCAGCAAGGAGAGC |
| | | ATCCTGCCCAAGCGGAACAGCGACAAGCTGATCGCCCGGAAGAAG |
| | | GACTGGGACCCCAAGAAGTACGGCGGCTTCGACAGCCCCACCGTGG |
| | | CCTACAGCGTGCTGGTGGTGGCCAAGGTGGAGAAGGGCAAGAGCA |
| | | AGAAGCTGAAGAGCGTGAAGGAGCTGCTGGGCATCACCATCATGG |
| | | AGCGGAGCAGCTTCGAGAAGAACCCCATCGACTTCCTGGAGGCCAA |
| | | GGGCTACAAGGAGGTGAAGAAGGACCTGATCATCAAGCTGCCCAA |
| | | GTACAGCCTGTTCGAGCTGGAGAACGGCCGGAAGCGGATGCTGGCC |
| | | AGCGCCGGCGAGCTGCAGAAGGGCAACGAGCTGGCCCTGCCCAGC |
| | | AAGTACGTGAACTTCCTGTACCTGGCCAGCCACTACGAGAAGCTGA |
| | | AGGGCAGCCCCGAGGACAACGAGCAGAAGCAGCTGTTCGTGGAGC |
| | | AGCACAAGCACTACCTGGACGAGATCATCGAGCAGATCAGCGAGTT |
| | | CAGCAAGCGGGTGATCCTGGCCGACGCCAACCTGGACAAGGTGCTG |
| | | AGCGCCTACAACAAGCACCGGGACAAGCCCATCCGGGAGCAGGCC |
| | | GAGAACATCATCCACCTGTTCACCCTGACCAACCTGGGCGCCCCCG |
| | | CCGCCTTCAAGTACTTCGACACCACCATCGACCGGAAGCGGTACAC |
| | | CAGCACCAAGGAGGTGCTGGACGCCACCCTGATCCACCAGAGCATC |
| | | ACCGGCCTGTACGAGACCCGGATCGACCTGAGCCAGCTGGGCGGCG |
| | | ACGGCGGCGGCAGCCCCAAGAAGAAGCGGAAGGTG |
| 57 | Cas9 nickase | GACAAGAAGTACAGCATCGGCCTGGCCATCGGCACCAACAGCGTG |
| | ORF using low | GGCTGGGCCGTGATCACCGACGAGTACAAGGTGCCCAGCAAGAAG |
| | A/U codons of | TTCAAGGTGCTGGGCAACACCGACCGGCACAGCATCAAGAAGAAC |
| | Table 4, with two | CTGATCGGCGCCCTGCTGTTCGACAGCGGCGAGACCGCCGAGGCCA |
| | C-terminal NLS | CCCGGCTGAAGCGGACCGCCCGGCGGCGGTACACCCGGCGGAAGA |
| | sequences (no | ACCGGATCTGCTACCTGCAGGAGATCTTCAGCAACGAGATGGCCAA |
| | start or stop | GGTGGACGACAGCTTCTTCCACCGGCTGGAGGAGAGCTTCCTGGTG |
| | codons; suitable | GAGGAGGACAAGAAGCACGAGCGGCACCCCATCTTCGGCAACATC |
| | for inclusion in | GTGGACGAGGTGGCCTACCACGAGAAGTACCCCACCATCTACCACC |
| | fusion protein | TGCGGAAGAAGCTGGTGGACAGCACCGACAAGGCCGACCTGCGGC |
| | coding sequence) | TGATCTACCTGGCCCTGGCCCACATGATCAAGTTCCGGGGCCACTTC |
| | | CTGATCGAGGGCGACCTGAACCCCGACAACAGCGACGTGGACAAG |
| | | CTGTTCATCCAGCTGGTGCAGACCTACAACCAGCTGTTCGAGGAGA |
| | | ACCCCATCAACGCCAGCGGCGTGGACGCCAAGGCCATCCTGAGCGC |
| | | CCGGCTGAGCAAGAGCCGGCGGCTGGAGAACCTGATCGCCCAGCT |
| | | GCCCGGCGAGAAGAAGAACGGCCTGTTCGGCAACCTGATCGCCCTG |
| | | AGCCTGGGCCTGACCCCCAACTTCAAGAGCAACTTCGACCTGGCCG |
| | | AGGACGCCAAGCTGCAGCTGAGCAAGGACACCTACGACGACGACC |
| | | TGGACAACCTGCTGGCCCAGATCGGCGACCAGTACGCCGACCTGTT |
| | | CCTGGCCGCCAAGAACCTGAGCGACGCCATCCTGCTGAGCGACATC |
| | | CTGCGGGTGAACACCGAGATCACCAAGGCCCCCCTGAGCGCCAGCA |
| | | TGATCAAGCGGTACGACGAGCACCACCAGGACCTGACCCTGCTGAA |
| | | GGCCCTGGTGCGGCAGCAGCTGCCCGAGAAGTACAAGGAGATCTTC |
| | | TTCGACCAGAGCAAGAACGGCTACGCCGGCTACATCGACGGCGGC |
| | | GCCAGCCAGGAGGAGTTCTACAAGTTCATCAAGCCCATCCTGGAGA |
| | | AGATGGACGGCACCGAGGAGCTGCTGGTGAAGCTGAACCGGGAGG |
| | | ACCTGCTGCGGAAGCAGCGGACCTTCGACAACGGCAGCATCCCCCA |
| | | CCAGATCCACCTGGGCGAGCTGCACGCCATCCTGCGGCGGCAGGAG |
| | | GACTTCTACCCCTTCCTGAAGGACAACCGGGAGAAGATCGAGAAGA |
| | | TCCTGACCTTCCGGATCCCCTACTACGTGGGCCCCCTGGCCCGGGGC |
| | | AACAGCCGGTTCGCCTGGATGACCCGGAAGAGCGAGGAGACCATC |
| | | ACCCCCTGGAACTTCGAGGAGGTGGTGGACAAGGGCGCCAGCGCC |
| | | CAGAGCTTCATCGAGCGGATGACCAACTTCGACAAGAACCTGCCCA |
| | | ACGAGAAGGTGCTGCCCAAGCACAGCCTGCTGTACGAGTACTTCAC |
| | | CGTGTACAACGAGCTGACCAAGGTGAAGTACGTGACCGAGGGCAT |
| | | GCGGAAGCCCGCCTTCCTGAGCGGCGAGCAGAAGAAGGCCATCGT |
| | | GGACCTGCTGTTCAAGACCAACCGGAAGGTGACCGTGAAGCAGCTG |
| | | AAGGAGGACTACTTCAAGAAGATCGAGTGCTTCGACAGCGTGGAG |
| | | ATCAGCGGCGTGGAGGACCGGTTCAACGCCAGCCTGGGCACCTACC |
| | | ACGACCTGCTGAAGATCATCAAGGACAAGGACTTCCTGGACAACGA |
| | | GGAGAACGAGGACATCCTGGAGGACATCGTGCTGACCCTGACCCTG |
| | | TTCGAGGACCGGGAGATGATCGAGGAGCGGCTGAAGACCTACGCC |
| | | CACCTGTTCGACGACAAGGTGATGAAGCAGCTGAAGCGGCGGCGG |
| | | TACACCGGCTGGGGCCGGCTGAGCCGGAAGCTGATCAACGGCATCC |
| | | GGGACAAGCAGAGCGGCAAGACCATCCTGGACTTCCTGAAGAGCG |
| | | ACGGCTTCGCCAACCGGAACTTCATGCAGCTGATCCACGACGACAG |
| | | CCTGACCTTCAAGGAGGACATCCAGAAGGCCCAGGTGAGCGGCCA |
| | | GGGCGACAGCCTGCACGAGCACATCGCCAACCTGGCCGGCAGCCCC |
| | | GCCATCAAGAAGGGCATCCTGCAGACCGTGAAGGTGGTGGACGAG |
| | | CTGGTGAAGGTGATGGGCCGGCACAAGCCCGAGAACATCGTGATC |
| | | GAGATGGCCCGGGAGAACCAGACCACCCAGAAGGGCCAGAAGAAC |
| | | AGCCGGGAGCGGATGAAGCGGATCGAGGAGGGCATCAAGGAGCTG |
| | | GGCAGCCAGATCCTGAAGGAGCACCCCGTGGAGAACACCCAGCTG |
| | | CAGAACGAGAAGCTGTACCTGTACTACCTGCAGAACGGCCGGGAC |
| | | ATGTACGTGGACCAGGAGCTGGACATCAACCGGCTGAGCGACTACG |
| | | ACGTGGACCACATCGTGCCCCAGAGCTTCCTGAAGGACGACAGCAT |
| | | CGACAACAAGGTGCTGACCCGGAGCGACAAGAACCGGGGCAAGAG |
| | | CGACAACGTGCCCAGCGAGGAGGTGGTGAAGAAGATGAAGAACTA |
| | | CTGGCGGCAGCTGCTGAACGCCAAGCTGATCACCCAGCGGAAGTTC |
| | | GACAACCTGACCAAGGCCGAGCGGGGCGGCCTGAGCGAGCTGGAC |
| | | AAGGCCGGCTTCATCAAGCGGCAGCTGGTGGAGACCCGGCAGATC |
| | | ACCAAGCACGTGGCCCAGATCCTGGACAGCCGGATGAACACCAAG |
| | | TACGACGAGAACGACAAGCTGATCCGGGAGGTGAAGGTGATCACC |
| | | CTGAAGAGCAAGCTGGTGAGCGACTTCCGGAAGGACTTCCAGTTCT |
| | | ACAAGGTGCGGGAGATCAACAACTACCACCACGCCCACGACGCCT |
| | | ACCTGAACGCCGTGGTGGGCACCGCCCTGATCAAGAAGTACCCCAA |
| | | GCTGGAGAGCGAGTTCGTGTACGGCGACTACAAGGTGTACGACGTG |
| | | CGGAAGATGATCGCCAAGAGCGAGCAGGAGATCGGCAAGGCCACC |
| | | GCCAAGTACTTCTTCTACAGCAACATCATGAACTTCTTCAAGACCG |
| | | AGATCACCCTGGCCAACGGCGAGATCCGGAAGCGGCCCCTGATCGA |
| | | GACCAACGGCGAGACCGGCGAGATCGTGTGGGACAAGGGCCGGGA |
| | | CTTCGCCACCGTGCGGAAGGTGCTGAGCATGCCCCAGGTGAACATC |
| | | GTGAAGAAGACCGAGGTGCAGACCGGCGGCTTCAGCAAGGAGAGC |
| | | ATCCTGCCCAAGCGGAACAGCGACAAGCTGATCGCCCGGAAGAAG |
| | | GACTGGGACCCCAAGAAGTACGGCGGCTTCGACAGCCCCACCGTGG |
| | | CCTACAGCGTGCTGGTGGTGGCCAAGGTGGAGAAGGGCAAGAGCA |
| | | AGAAGCTGAAGAGCGTGAAGGAGCTGCTGGGCATCACCATCATGG |
| | | AGCGGAGCAGCTTCGAGAAGAACCCCATCGACTTCCTGGAGGCCAA |
| | | GGGCTACAAGGAGGTGAAGAAGGACCTGATCATCAAGCTGCCCAA |
| | | GTACAGCCTGTTCGAGCTGGAGAACGGCCGGAAGCGGATGCTGGCC |
| | | AGCGCCGGCGAGCTGCAGAAGGGCAACGAGCTGGCCCTGCCCAGC |
| | | AAGTACGTGAACTTCCTGTACCTGGCCAGCCACTACGAGAAGCTGA |
| | | AGGGCAGCCCCGAGGACAACGAGCAGAAGCAGCTGTTCGTGGAGC |
| | | AGCACAAGCACTACCTGGACGAGATCATCGAGCAGATCAGCGAGTT |
| | | CAGCAAGCGGGTGATCCTGGCCGACGCCAACCTGGACAAGGTGCTG |
| | | AGCGCCTACAACAAGCACCGGGACAAGCCCATCCGGGAGCAGGCC |
| | | GAGAACATCATCCACCTGTTCACCCTGACCAACCTGGGCGCCCCCG |
| | | CCGCCTTCAAGTACTTCGACACCACCATCGACCGGAAGCGGTACAC |
| | | CAGCACCAAGGAGGTGCTGGACGCCACCCTGATCCACCAGAGCATC |
| | | ACCGGCCTGTACGAGACCCGGATCGACCTGAGCCAGCTGGGCGGCG |
| | | ACGGCAGCGGCAGCCCCAAGAAGAAGCGGAAGGTGGACGGCAGCC |
| | | CCAAGAAGAAGCGGAAGGTGGACAGCGGC |
| 58 | Cas9 nickase | GACAAGAAGTACAGCATCGGCCTGGCCATCGGCACCAACAGCGTG |
| | ORF using low | GGCTGGGCCGTGATCACCGACGAGTACAAGGTGCCCAGCAAGAAG |
| | A/U codons of | TTCAAGGTGCTGGGCAACACCGACCGGCACAGCATCAAGAAGAAC |
| | Table 4 (no NLS | CTGATCGGCGCCCTGCTGTTCGACAGCGGCGAGACCGCCGAGGCCA |
| | and no start or | CCCGGCTGAAGCGGACCGCCCGGCGGCGGTACACCCGGCGGAAGA |
| | stop codons; | ACCGGATCTGCTACCTGCAGGAGATCTTCAGCAACGAGATGGCCAA |
| | suitable for | GGTGGACGACAGCTTCTTCCACCGGCTGGAGGAGAGCTTCCTGGTG |
| | inclusion in | GAGGAGGACAAGAAGCACGAGCGGCACCCCATCTTCGGCAACATC |
| | fusion protein | GTGGACGAGGTGGCCTACCACGAGAAGTACCCCACCATCTACCACC |
| | coding sequence) | TGCGGAAGAAGCTGGTGGACAGCACCGACAAGGCCGACCTGCGGC |
| | | TGATCTACCTGGCCCTGGCCCACATGATCAAGTTCCGGGGCCACTTC |
| | | CTGATCGAGGGCGACCTGAACCCCGACAACAGCGACGTGGACAAG |
| | | CTGTTCATCCAGCTGGTGCAGACCTACAACCAGCTGTTCGAGGAGA |
| | | ACCCCATCAACGCCAGCGGCGTGGACGCCAAGGCCATCCTGAGCGC |
| | | CCGGCTGAGCAAGAGCCGGCGGCTGGAGAACCTGATCGCCCAGCT |
| | | GCCCGGCGAGAAGAAGAACGGCCTGTTCGGCAACCTGATCGCCCTG |
| | | AGCCTGGGCCTGACCCCCAACTTCAAGAGCAACTTCGACCTGGCCG |
| | | AGGACGCCAAGCTGCAGCTGAGCAAGGACACCTACGACGACGACC |
| | | TGGACAACCTGCTGGCCCAGATCGGCGACCAGTACGCCGACCTGTT |
| | | CCTGGCCGCCAAGAACCTGAGCGACGCCATCCTGCTGAGCGACATC |
| | | CTGCGGGTGAACACCGAGATCACCAAGGCCCCCCTGAGCGCCAGCA |
| | | TGATCAAGCGGTACGACGAGCACCACCAGGACCTGACCCTGCTGAA |
| | | GGCCCTGGTGCGGCAGCAGCTGCCCGAGAAGTACAAGGAGATCTTC |
| | | TTCGACCAGAGCAAGAACGGCTACGCCGGCTACATCGACGGCGGC |
| | | GCCAGCCAGGAGGAGTTCTACAAGTTCATCAAGCCCATCCTGGAGA |
| | | AGATGGACGGCACCGAGGAGCTGCTGGTGAAGCTGAACCGGGAGG |
| | | ACCTGCTGCGGAAGCAGCGGACCTTCGACAACGGCAGCATCCCCCA |
| | | CCAGATCCACCTGGGCGAGCTGCACGCCATCCTGCGGCGGCAGGAG |
| | | GACTTCTACCCCTTCCTGAAGGACAACCGGGAGAAGATCGAGAAGA |
| | | TCCTGACCTTCCGGATCCCCTACTACGTGGGCCCCCTGGCCCGGGGC |
| | | AACAGCCGGTTCGCCTGGATGACCCGGAAGAGCGAGGAGACCATC |
| | | ACCCCCTGGAACTTCGAGGAGGTGGTGGACAAGGGCGCCAGCGCC |
| | | CAGAGCTTCATCGAGCGGATGACCAACTTCGACAAGAACCTGCCCA |
| | | ACGAGAAGGTGCTGCCCAAGCACAGCCTGCTGTACGAGTACTTCAC |
| | | CGTGTACAACGAGCTGACCAAGGTGAAGTACGTGACCGAGGGCAT |
| | | GCGGAAGCCCGCCTTCCTGAGCGGCGAGCAGAAGAAGGCCATCGT |
| | | GGACCTGCTGTTCAAGACCAACCGGAAGGTGACCGTGAAGCAGCTG |
| | | AAGGAGGACTACTTCAAGAAGATCGAGTGCTTCGACAGCGTGGAG |
| | | ATCAGCGGCGTGGAGGACCGGTTCAACGCCAGCCTGGGCACCTACC |
| | | ACGACCTGCTGAAGATCATCAAGGACAAGGACTTCCTGGACAACGA |
| | | GGAGAACGAGGACATCCTGGAGGACATCGTGCTGACCCTGACCCTG |
| | | TTCGAGGACCGGGAGATGATCGAGGAGCGGCTGAAGACCTACGCC |
| | | CACCTGTTCGACGACAAGGTGATGAAGCAGCTGAAGCGGCGGCGG |
| | | TACACCGGCTGGGGCCGGCTGAGCCGGAAGCTGATCAACGGCATCC |
| | | GGGACAAGCAGAGCGGCAAGACCATCCTGGACTTCCTGAAGAGCG |
| | | ACGGCTTCGCCAACCGGAACTTCATGCAGCTGATCCACGACGACAG |
| | | CCTGACCTTCAAGGAGGACATCCAGAAGGCCCAGGTGAGCGGCCA |
| | | GGGCGACAGCCTGCACGAGCACATCGCCAACCTGGCCGGCAGCCCC |
| | | GCCATCAAGAAGGGCATCCTGCAGACCGTGAAGGTGGTGGACGAG |
| | | CTGGTGAAGGTGATGGGCCGGCACAAGCCCGAGAACATCGTGATC |
| | | GAGATGGCCCGGGAGAACCAGACCACCCAGAAGGGCCAGAAGAAC |
| | | AGCCGGGAGCGGATGAAGCGGATCGAGGAGGGCATCAAGGAGCTG |
| | | GGCAGCCAGATCCTGAAGGAGCACCCCGTGGAGAACACCCAGCTG |
| | | CAGAACGAGAAGCTGTACCTGTACTACCTGCAGAACGGCCGGGAC |
| | | ATGTACGTGGACCAGGAGCTGGACATCAACCGGCTGAGCGACTACG |
| | | ACGTGGACCACATCGTGCCCCAGAGCTTCCTGAAGGACGACAGCAT |
| | | CGACAACAAGGTGCTGACCCGGAGCGACAAGAACCGGGGCAAGAG |
| | | CGACAACGTGCCCAGCGAGGAGGTGGTGAAGAAGATGAAGAACTA |
| | | CTGGCGGCAGCTGCTGAACGCCAAGCTGATCACCCAGCGGAAGTTC |
| | | GACAACCTGACCAAGGCCGAGCGGGGCGGCCTGAGCGAGCTGGAC |
| | | AAGGCCGGCTTCATCAAGCGGCAGCTGGTGGAGACCCGGCAGATC |
| | | ACCAAGCACGTGGCCCAGATCCTGGACAGCCGGATGAACACCAAG |
| | | TACGACGAGAACGACAAGCTGATCCGGGAGGTGAAGGTGATCACC |
| | | CTGAAGAGCAAGCTGGTGAGCGACTTCCGGAAGGACTTCCAGTTCT |
| | | ACAAGGTGCGGGAGATCAACAACTACCACCACGCCCACGACGCCT |
| | | ACCTGAACGCCGTGGTGGGCACCGCCCTGATCAAGAAGTACCCCAA |
| | | GCTGGAGAGCGAGTTCGTGTACGGCGACTACAAGGTGTACGACGTG |
| | | CGGAAGATGATCGCCAAGAGCGAGCAGGAGATCGGCAAGGCCACC |
| | | GCCAAGTACTTCTTCTACAGCAACATCATGAACTTCTTCAAGACCG |
| | | AGATCACCCTGGCCAACGGCGAGATCCGGAAGCGGCCCCTGATCGA |
| | | GACCAACGGCGAGACCGGCGAGATCGTGTGGGACAAGGGCCGGGA |
| | | CTTCGCCACCGTGCGGAAGGTGCTGAGCATGCCCCAGGTGAACATC |
| | | GTGAAGAAGACCGAGGTGCAGACCGGCGGCTTCAGCAAGGAGAGC |
| | | ATCCTGCCCAAGCGGAACAGCGACAAGCTGATCGCCCGGAAGAAG |
| | | GACTGGGACCCCAAGAAGTACGGCGGCTTCGACAGCCCCACCGTGG |
| | | CCTACAGCGTGCTGGTGGTGGCCAAGGTGGAGAAGGGCAAGAGCA |
| | | AGAAGCTGAAGAGCGTGAAGGAGCTGCTGGGCATCACCATCATGG |
| | | AGCGGAGCAGCTTCGAGAAGAACCCCATCGACTTCCTGGAGGCCAA |
| | | GGGCTACAAGGAGGTGAAGAAGGACCTGATCATCAAGCTGCCCAA |
| | | GTACAGCCTGTTCGAGCTGGAGAACGGCCGGAAGCGGATGCTGGCC |
| | | AGCGCCGGCGAGCTGCAGAAGGGCAACGAGCTGGCCCTGCCCAGC |
| | | AAGTACGTGAACTTCCTGTACCTGGCCAGCCACTACGAGAAGCTGA |
| | | AGGGCAGCCCCGAGGACAACGAGCAGAAGCAGCTGTTCGTGGAGC |
| | | AGCACAAGCACTACCTGGACGAGATCATCGAGCAGATCAGCGAGTT |
| | | CAGCAAGCGGGTGATCCTGGCCGACGCCAACCTGGACAAGGTGCTG |
| | | AGCGCCTACAACAAGCACCGGGACAAGCCCATCCGGGAGCAGGCC |
| | | GAGAACATCATCCACCTGTTCACCCTGACCAACCTGGGCGCCCCCG |
| | | CCGCCTTCAAGTACTTCGACACCACCATCGACCGGAAGCGGTACAC |
| | | CAGCACCAAGGAGGTGCTGGACGCCACCCTGATCCACCAGAGCATC |
| | | ACCGGCCTGTACGAGACCCGGATCGACCTGAGCCAGCTGGGCGGCG |
| | | AC |
| 59 | Exemplary NLS 1 | LAAKRSRTT |
| 60 | Exemplary NLS 2 | QAAKRSRTT |
| 61 | Exemplary NLS 3 | PAPAKRERTT |
| 62 | Exemplary NLS 4 | QAAKRPRTT |
| 63 | Exemplary NLS 5 | RAAKRPRTT |
| 64 | Exemplary NLS 6 | AAAKRSWSMAA |
| 65 | Exemplary NLS 7 | AAAKRVWSMAF |
| 66 | Exemplary NLS 8 | AAAKRSWSMAF |
| 67 | Exemplary NLS 9 | AAAKRKYFAA |
| 68 | Exemplary NLS | RAAKRKAFAA |
| | 10 | |
| 69 | Exemplary NLS | RAAKRKYFAV |
| | 11 | |
| 70 | Alternate SV40 | PKKKRRV |
| | NLS | |
| 71 | Nucleoplasmin | KRPAATKKAGQAKKKK |
| | NLS | |
| 72 | amino acid | GGS |
| | sequence for | |
| | exemplary linker | |
| 73 | amino acid | GGGGS |
| | sequence for | |
| | exemplary linker | |
| 74 | amino acid | EAAAK |
| | sequence for | |
| | exemplary linker | |
| 75 | amino acid | SEGSA |
| | sequence for | |
| | exemplary linker | |
| 76 | amino acid | SEGSAGTST |
| | sequence for | |
| | exemplary linker | |
| 77 | amino acid | GGGGSGGGGS |
| | sequence for | |
| | exemplary linker | |
| 78 | amino acid | GGGGSEAAAK |
| | sequence for | |
| | exemplary linker | |
| 79 | amino acid | EAAAKGGGGS |
| | sequence for | |
| | exemplary linker | |
| 80 | amino acid | EAAAKEAAAK |
| | sequence for | |
| | exemplary linker | |
| 81 | amino acid | SEGSAGTSTESEGSA |
| | sequence for | |
| | exemplary linker | |
| 82 | amino acid | GGGGSGGGGSGGGGS |
| | sequence for | |
| | exemplary linker | |
| 83 | amino acid | GGGGSGGGGSEAAAK |
| | sequence for | |
| | exemplary linker | |
| 84 | amino acid | GGGGSEAAAKGGGGS |
| | sequence for | |
| | exemplary linker | |
| 85 | amino acid | EAAAKGGGGSEAAAK |
| | sequence for | |
| | exemplary linker | |
| 86 | amino acid | EAAAKEAAAKGGGGS |
| | sequence for | |
| | exemplary linker | |
| 87 | amino acid | SEGSAGTSTESEGSAGTSTE |
| | sequence for | |
| | exemplary linker | |
| 88 | amino acid | GGGGSGGGGSGGGGSEAAAK |
| | sequence for | |
| | exemplary linker | |
| 89 | amino acid | GGGGSGGGGSEAAAKGGGGS |
| | sequence for | |
| | exemplary linker | |
| 90 | amino acid | GGGGSEAAAKGGGGSEAAAK |
| | sequence for | |
| | exemplary linker | |
| 91 | amino acid | GGGGSEAAAKEAAAKGGGGS |
| | sequence for | |
| | exemplary linker | |
| 92 | amino acid | GGGGSEAAAKEAAAKEAAAK |
| | sequence for | |
| | exemplary linker | |
| 93 | amino acid | EAAAKGGGGSGGGGSGGGGS |
| | sequence for | |
| | exemplary linker | |
| 94 | amino acid | EAAAKGGGGSGGGGSEAAAK |
| | sequence for | |
| | exemplary linker | |
| 95 | amino acid | EAAAKGGGGSEAAAKGGGGS |
| | sequence for | |
| | exemplary linker | |
| 96 | amino acid | EAAAKGGGGSEAAAKEAAAK |
| | sequence for | |
| | exemplary linker | |
| 97 | amino acid | EAAAKEAAAKGGGGGGGGS |
| | sequence for | |
| | exemplary linker | |
| 98 | amino acid | EAAAKEAAAKGGGGSEAAAK |
| | sequence for | |
| | exemplary linker | |
| 99 | amino acid | EAAAKEAAAKEAAAKGGGGS |
| | sequence for | |
| | exemplary linker | |
| 100 | amino acid | SEGSAGTSTESEGSAGTSTESEGSA |
| | sequence for | |
| | exemplary linker | |
| 101 | amino acid | GGGGSGGGGSGGGGSGGGGSGGGGS |
| | sequence for | |
| | exemplary linker | |
| 102 | amino acid | GGGGSGGGGSGGGGSGGGGSEAAAK |
| | sequence for | |
| | exemplary linker | |
| 103 | amino acid | GGGGSGGGGSGGGGSEAAAKGGGGS |
| | sequence for | |
| | exemplary linker | |
| 104 | amino acid | GGGGSGGGGSGGGGSEAAAKEAAAK |
| | sequence for | |
| | exemplary linker | |
| 105 | amino acid | GGGGSGGGGSEAAAKGGGGSGGGGS |
| | sequence for | |
| | exemplary linker | |
| 106 | amino acid | GGGGSGGGGSEAAAKGGGGSEAAAK |
| | sequence for | |
| | exemplary linker | |
| 107 | amino acid | GGGGSGGGGSEAAAKEAAAKGGGGS |
| | sequence for | |
| | exemplary linker | |
| 108 | amino acid | GGGGSGGGGSEAAAKEAAAKEAAAK |
| | sequence for | |
| | exemplary linker | |
| 109 | amino acid | GGGGSEAAAKGGGGSGGGGSGGGGS |
| | sequence for | |
| | exemplary linker | |
| 110 | amino acid | GGGGSEAAAKGGGGSGGGGSEAAAK |
| | sequence for | |
| | exemplary linker | |
| 111 | amino acid | GGGGSEAAAKGGGGSEAAAKGGGGS |
| | sequence for | |
| | exemplary linker | |
| 112 | amino acid | GGGGSEAAAKGGGGSEAAAKEAAAK |
| | sequence for | |
| | exemplary linker | |
| 113 | amino acid | GGGGSEAAAKEAAAKGGGGSGGGGS |
| | sequence for | |
| | exemplary linker | |
| 114 | amino acid | GGGGSEAAAKEAAAKEAAAKGGGGS |
| | sequence for | |
| | exemplary linker | |
| 115 | amino acid | GGGGSEAAAKEAAAKEAAAKEAAAK |
| | sequence for | |
| | exemplary linker | |
| 116 | amino acid | EAAAKGGGGSGGGGSGGGGSGGGGS |
| | sequence for | |
| | exemplary linker | |
| 117 | amino acid | EAAAKGGGGSGGGGSGGGGSEAAAK |
| | sequence for | |
| | exemplary linker | |
| 118 | amino acid | EAAAKGGGGGGGGSEAAAKGGGGS |
| | sequence for | |
| | exemplary linker | |
| 119 | amino acid | EAAAKGGGGSGGGGSEAAAKEAAAK |
| | sequence for | |
| | exemplary linker | |
| 120 | amino acid | EAAAKGGGGSEAAAKGGGGSGGGGS |
| | sequence for | |
| | exemplary linker | |
| 121 | amino acid | EAAAKGGGGSEAAAKGGGGSEAAAK |
| | sequence for | |
| | exemplary linker | |
| 122 | amino acid | EAAAKGGGGSEAAAKEAAAKGGGGS |
| | sequence for | |
| | exemplary linker | |
| 123 | amino acid | EAAAKGGGGSEAAAKEAAAKEAAAK |
| | sequence for | |
| | exemplary linker | |
| 124 | amino acid | EAAAKEAAAKGGGGSEAAAKGGGGS |
| | sequence for | |
| | exemplary linker | |
| 125 | amino acid | EAAAKEAAAKGGGGSEAAAKEAAAK |
| | sequence for | |
| | exemplary linker | |
| 126 | amino acid | EAAAKEAAAKEAAAKGGGGSEAAAK |
| | sequence for | |
| | exemplary linker | |
| 127 | amino acid | EAAAKEAAAKEAAAKEAAAKGGGGS |
| | sequence for | |
| | exemplary linker | |
| 128 | amino acid | EAAAKEAAAKEAAAKEAAAKEAAAK |
| | sequence for | |
| | exemplary linker | |
| 129 | amino acid | GTKDSTKDIPETPSKD |
| | sequence for | |
| | exemplary linker | |
| 130 | amino acid | GRDVRQPEVKEEKPES |
| | sequence for | |
| | exemplary linker | |
| 131 | amino acid | EGKSSGSGSESKSTAG |
| | sequence for | |
| | exemplary linker | |
| 132 | amino acid | TPGSPAGSPTSTEEGT |
| | sequence for | |
| | exemplary linker | |
| 133 | amino acid | GSEPATSGSETPGTST |
| | sequence for | |
| | exemplary linker | |
| 134 | Exemplary | GGGAAGCUCAGAAUAAACGCUCAACUUUGGCCGGAUCUGCCACCA |
| | mRNA encoding | UGGAGGCCUCCCCCGCCUCCGGCCCCCGGCACCUGAUGGACCCCC |
| | APOBEC3A- | ACAUCUUCACCUCCAACUUCAACAACGGCAUCGGCCGGCACAAGA |
| | Nme2D16A | CCUACCUGUGCUACGAGGUGGAGCGGCUGGACAACGGCACCUCCG |
| | | UGAAGAUGGACCAGCACCGGGGCUUCCUGCACAACCAGGCCAAGA |
| | | ACCUGCUGUGCGGCUUCUACGGCCGGCACGCCGAGCUGCGGUUCC |
| | | UGGACCUGGUGCCCUCCCUGCAGCUGGACCCCGCCCAGAUCUACC |
| | | GGGUGACCUGGUUCAUCUCCUGGUCCCCCUGCUUCUCCUGGGGCU |
| | | GCGCCGGCGAGGUGCGGGCCUUCCUGCAGGAGAACACCCACGUGC |
| | | GGCUGCGGAUCUUCGCCGCCCGGAUCUACGACUACGACCCCCUGU |
| | | ACAAGGAGGCCCUGCAGAUGCUGCGGGACGCCGGCGCCCAGGUGU |
| | | CCAUCAUGACCUACGACGAGUUCAAGCACUGCUGGGACACCUUCG |
| | | UGGACCACCAGGGCUGCCCCUUCCAGCCCUGGGACGGCCUGGACG |
| | | AGCACUCCCAGGCCCUGUCCGGCCGGCUGCGGGCCAUCCUGCAGA |
| | | ACCAGGGCAACUCCGGCUCCGAGACCCCCGGCACCUCCGAGUCCG |
| | | CCACCCCCGAGUCCGCAGCGUUCAAACCAAAUCCCAUCAACUACA |
| | | UCCUGGGCCUGGCCAUCGGCAUCGCCUCCGUGGGCUGGGCCAUGG |
| | | UGGAGAUCGACGAGGAGGAGAACCCCAUCCGGCUGAUCGACCUG |
| | | GGCGUGCGGGUGUUCGAGCGGGCCGAGGUGCCCAAGACCGGCGAC |
| | | UCCCUGGCCAUGGCCCGGCGGCUGGCCCGGUCCGUGCGGCGGCUG |
| | | ACCCGGCGGCGGGCCCACCGGCUGCUGCGGGCCCGGCGGCUGCUG |
| | | AAGCGGGAGGGCGUGCUGCAGGCCGCCGACUUCGACGAGAACGGC |
| | | CUGAUCAAGUCCCUGCCCAACACCCCCUGGCAGCUGCGGGCCGCC |
| | | GCCCUGGACCGGAAGCUGACCCCCCUGGAGUGGUCCGCCGUGCUG |
| | | CUGCACCUGAUCAAGCACCGGGGCUACCUGUCCCAGCGGAAGAAC |
| | | GAGGGCGAGACCGCCGACAAGGAGCUGGGCGCCCUGCUGAAGGGC |
| | | GUGGCCAACAACGCCCACGCCCUGCAGACCGGCGACUUCCGGACC |
| | | CCCGCCGAGCUGGCCCUGAACAAGUUCGAGAAGGAGUCCGGCCAC |
| | | AUCCGGAACCAGCGGGGCGACUACUCCCACACCUUCUCCCGGAAG |
| | | GACCUGCAGGCCGAGCUGAUCCUGCUGUUCGAGAAGCAGAAGGA |
| | | GUUCGGCAACCCCCACGUGUCCGGCGGCCUGAAGGAGGGCAUCGA |
| | | GACCCUGCUGAUGACCCAGCGGCCCGCCCUGUCCGGCGACGCCGU |
| | | GCAGAAGAUGCUGGGCCACUGCACCUUCGAGCCCGCCGAGCCCAA |
| | | GGCCGCCAAGAACACCUACACCGCCGAGCGGUUCAUCUGGCUGAC |
| | | CAAGCUGAACAACCUGCGGAUCCUGGAGCAGGGCUCCGAGCGGCC |
| | | CCUGACCGACACCGAGCGGGCCACCCUGAUGGACGAGCCCUACCG |
| | | GAAGUCCAAGCUGACCUACGCCCAGGCCCGGAAGCUGCUGGGCCU |
| | | GGAGGACACCGCCUUCUUCAAGGGCCUGCGGUACGGCAAGGACAA |
| | | CGCCGAGGCCUCCACCCUGAUGGAGAUGAAGGCCUACCACGCCAU |
| | | CUCCCGGGCCCUGGAGAAGGAGGGCCUGAAGGACAAGAAGUCCCC |
| | | CCUGAACCUGUCCUCCGAGCUGCAGGACGAGAUCGGCACCGCCUU |
| | | CUCCCUGUUCAAGACCGACGAGGACAUCACCGGCCGGCUGAAGGA |
| | | CCGGGUGCAGCCCGAGAUCCUGGAGGCCCUGCUGAAGCACAUCUC |
| | | CUUCGACAAGUUCGUGCAGAUCUCCCUGAAGGCCCUGCGGCGGAU |
| | | CGUGCCCCUGAUGGAGCAGGGCAAGCGGUACGACGAGGCCUGCGC |
| | | CGAGAUCUACGGCGACCACUACGGCAAGAAGAACACCGAGGAGA |
| | | AGAUCUACCUGCCCCCCAUCCCCGCCGACGAGAUCCGGAACCCCG |
| | | UGGUGCUGCGGGCCCUGUCCCAGGCCCGGAAGGUGAUCAACGGCG |
| | | UGGUGCGGCGGUACGGCUCCCCCGCCCGGAUCCACAUCGAGACCG |
| | | CCCGGGAGGUGGGCAAGUCCUUCAAGGACCGGAAGGAGAUCGAG |
| | | AAGCGGCAGGAGGAGAACCGGAAGGACCGGGAGAAGGCCGCCGC |
| | | CAAGUUCCGGGAGUACUUCCCCAACUUCGUGGGCGAGCCCAAGUC |
| | | CAAGGACAUCCUGAAGCUGCGGCUGUACGAGCAGCAGCACGGCAA |
| | | GUGCCUGUACUCCGGCAAGGAGAUCAACCUGGUGCGGCUGAACG |
| | | AGAAGGGCUACGUGGAGAUCGACCACGCCCUGCCCUUCUCCCGGA |
| | | CCUGGGACGACUCCUUCAACAACAAGGUGCUGGUGCUGGGCUCCG |
| | | AGAACCAGAACAAGGGCAACCAGACCCCCUACGAGUACUUCAACG |
| | | GCAAGGACAACUCCCGGGAGUGGCAGGAGUUCAAGGCCCGGGUG |
| | | GAGACCUCCCGGUUCCCCCGGUCCAAGAAGCAGCGGAUCCUGCUG |
| | | CAGAAGUUCGACGAGGACGGCUUCAAGGAGUGCAACCUGAACGA |
| | | CACCCGGUACGUGAACCGCUUCCUGUGCCAGUUCGUGGCCGACCA |
| | | CAUCCUGCUGACCGGCAAGGGCAAGCGGCGGGUGUUCGCCUCCAA |
| | | CGGCCAGAUCACCAACCUGCUGCGGGGCUUCUGGGGCCUGCGGAA |
| | | GGUGCGGGCCGAGAACGACCGGCACCACGCCCUGGACGCCGUGGU |
| | | GGUGGCCUGCUCCACCGUGGCCAUGCAGCAGAAGAUCACCCGGUU |
| | | CGUGCGGUACAAGGAGAUGAACGCCUUCGACGGCAAGACCAUCG |
| | | ACAAGGAGACCGGCAAGGUGCUGCACCAGAAGACCCACUUCCCCC |
| | | AGCCCUGGGAGUUCUUCGCCCAGGAGGUGAUGAUCCGGGUGUUC |
| | | GGCAAGCCCGACGGCAAGCCCGAGUUCGAGGAGGCCGACACCCCC |
| | | GAGAAGCUGCGGACCCUGCUGGCCGAGAAGCUGUCCUCCCGGCCC |
| | | GAGGCCGUGCACGAGUACGUGACCCCCCUGUUCGUGUCCCGGGCC |
| | | CCCAACCGGAAGAUGUCCGGCGCCCACAAGGACACCCUGCGGUCC |
| | | GCCAAGCGGUUCGUGAAGCACAACGAGAAGAUCUCCGUGAAGCG |
| | | GGUGUGGCUGACCGAGAUCAAGCUGGCCGACCUGGAGAACAUGG |
| | | UGAACUACAAGAACGGCCGGGAGAUCGAGCUGUACGAGGCCCUG |
| | | AAGGCCCGGCUGGAGGCCUACGGCGGCAACGCCAAGCAGGCCUUC |
| | | GACCCCAAGGACAACCCCUUCUACAAGAAGGGCGGCCAGCUGGUG |
| | | AAGGCCGUGCGGGUGGAGAAGACCCAGGAGUCCGGCGUGCUGCU |
| | | GAACAAGAAGAACGCCUACACCAUCGCCGACAACGGCGACAUGGU |
| | | GCGGGUGGACGUGUUCUGCAAGGUGGACAAGAAGGGCAAGAACC |
| | | AGUACUUCAUCGUGCCCAUCUACGCCUGGCAGGUGGCCGAGAACA |
| | | UCCUGCCCGACAUCGACUGCAAGGGCUACCGGAUCGACGACUCCU |
| | | ACACCUUCUGCUUCUCCCUGCACAAGUACGACCUGAUCGCCUUCC |
| | | AGAAGGACGAGAAGUCCAAGGUGGAGUUCGCCUACUACAUCAAC |
| | | UGCGACUCCUCCAACGGCCGGUUCUACCUGGCCUGGCACGACAAG |
| | | GGCUCCAAGGAGCAGCAGUUCCGGAUCUCCACCCAGAACCUGGUG |
| | | CUGAUCCAGAAGUACCAGGUGAACGAGCUGGGCAAGGAGAUCCG |
| | | GCCCUGCCGGCUGAAGAAGCGGCCCCCCGUGCGGUCCGGAAAGCG |
| | | GACCGCCGACGGCUCCGAGUUCGAGUCCCCCAAGAAGAAGCGGAA |
| | | GGUGGAGUAGUGACUAGCACCAGCCUCAAGAACACCCGAAUGGA |
| | | GUCUCUAAGCUACAUAAUACCAACUUACACUUUACAAAAUGUUG |
| | | UCCCCCAAAAUGUAGCCAUUCGUAUCUGCUCCUAAUAAAAAGAA |
| | | AGUUUCUUCACAUUCU |
| 135 | Exemplary open | AUGGAGGCCUCCCCCGCCUCCGGCCCCCGGCACCUGAUGGACCCC |
| | reading frame for | CACAUCUUCACCUCCAACUUCAACAACGGCAUCGGCCGGCACAAG |
| | APOBEC3A- | ACCUACCUGUGCUACGAGGUGGAGCGGCUGGACAACGGCACCUCC |
| | Nme2D16A | GUGAAGAUGGACCAGCACCGGGGCUUCCUGCACAACCAGGCCAAG |
| | | AACCUGCUGUGCGGCUUCUACGGCCGGCACGCCGAGCUGCGGUUC |
| | | CUGGACCUGGUGCCCUCCCUGCAGCUGGACCCCGCCCAGAUCUAC |
| | | CGGGUGACCUGGUUCAUCUCCUGGUCCCCCUGCUUCUCCUGGGGC |
| | | UGCGCCGGCGAGGUGCGGGCCUUCCUGCAGGAGAACACCCACGUG |
| | | CGGCUGCGGAUCUUCGCCGCCCGGAUCUACGACUACGACCCCCUG |
| | | UACAAGGAGGCCCUGCAGAUGCUGCGGGACGCCGGCGCCCAGGUG |
| | | UCCAUCAUGACCUACGACGAGUUCAAGCACUGCUGGGACACCUUC |
| | | GUGGACCACCAGGGCUGCCCCUUCCAGCCCUGGGACGGCCUGGAC |
| | | GAGCACUCCCAGGCCCUGUCCGGCCGGCUGCGGGCCAUCCUGCAG |
| | | AACCAGGGCAACUCCGGCUCCGAGACCCCCGGCACCUCCGAGUCC |
| | | GCCACCCCCGAGUCCGCAGCGUUCAAACCAAAUCCCAUCAACUAC |
| | | AUCCUGGGCCUGGCCAUCGGCAUCGCCUCCGUGGGCUGGGCCAUG |
| | | GUGGAGAUCGACGAGGAGGAGAACCCCAUCCGGCUGAUCGACCU |
| | | GGGCGUGCGGGUGUUCGAGCGGGCCGAGGUGCCCAAGACCGGCG |
| | | ACUCCCUGGCCAUGGCCCGGCGGCUGGCCCGGUCCGUGCGGCGGC |
| | | UGACCCGGCGGCGGGCCCACCGGCUGCUGCGGGCCCGGCGGCUGC |
| | | UGAAGCGGGAGGGCGUGCUGCAGGCCGCCGACUUCGACGAGAAC |
| | | GGCCUGAUCAAGUCCCUGCCCAACACCCCCUGGCAGCUGCGGGCC |
| | | GCCGCCCUGGACCGGAAGCUGACCCCCCUGGAGUGGUCCGCCGUG |
| | | CUGCUGCACCUGAUCAAGCACCGGGGCUACCUGUCCCAGCGGAAG |
| | | AACGAGGGCGAGACCGCCGACAAGGAGCUGGGCGCCCUGCUGAAG |
| | | GGCGUGGCCAACAACGCCCACGCCCUGCAGACCGGCGACUUCCGG |
| | | ACCCCCGCCGAGCUGGCCCUGAACAAGUUCGAGAAGGAGUCCGGC |
| | | CACAUCCGGAACCAGCGGGGCGACUACUCCCACACCUUCUCCCGG |
| | | AAGGACCUGCAGGCCGAGCUGAUCCUGCUGUUCGAGAAGCAGAA |
| | | GGAGUUCGGCAACCCCCACGUGUCCGGCGGCCUGAAGGAGGGCAU |
| | | CGAGACCCUGCUGAUGACCCAGCGGCCCGCCCUGUCCGGCGACGC |
| | | CGUGCAGAAGAUGCUGGGCCACUGCACCUUCGAGCCCGCCGAGCC |
| | | CAAGGCCGCCAAGAACACCUACACCGCCGAGCGGUUCAUCUGGCU |
| | | GACCAAGCUGAACAACCUGCGGAUCCUGGAGCAGGGCUCCGAGCG |
| | | GCCCCUGACCGACACCGAGCGGGCCACCCUGAUGGACGAGCCCUA |
| | | CCGGAAGUCCAAGCUGACCUACGCCCAGGCCCGGAAGCUGCUGGG |
| | | CCUGGAGGACACCGCCUUCUUCAAGGGCCUGCGGUACGGCAAGGA |
| | | CAACGCCGAGGCCUCCACCCUGAUGGAGAUGAAGGCCUACCACGC |
| | | CAUCUCCCGGGCCCUGGAGAAGGAGGGCCUGAAGGACAAGAAGU |
| | | CCCCCCUGAACCUGUCCUCCGAGCUGCAGGACGAGAUCGGCACCG |
| | | CCUUCUCCCUGUUCAAGACCGACGAGGACAUCACCGGCCGGCUGA |
| | | AGGACCGGGUGCAGCCCGAGAUCCUGGAGGCCCUGCUGAAGCACA |
| | | UCUCCUUCGACAAGUUCGUGCAGAUCUCCCUGAAGGCCCUGCGGC |
| | | GGAUCGUGCCCCUGAUGGAGCAGGGCAAGCGGUACGACGAGGCC |
| | | UGCGCCGAGAUCUACGGCGACCACUACGGCAAGAAGAACACCGAG |
| | | GAGAAGAUCUACCUGCCCCCCAUCCCCGCCGACGAGAUCCGGAAC |
| | | CCCGUGGUGCUGCGGGCCCUGUCCCAGGCCCGGAAGGUGAUCAAC |
| | | GGCGUGGUGCGGCGGUACGGCUCCCCCGCCCGGAUCCACAUCGAG |
| | | ACCGCCCGGGAGGUGGGCAAGUCCUUCAAGGACCGGAAGGAGAU |
| | | CGAGAAGCGGCAGGAGGAGAACCGGAAGGACCGGGAGAAGGCCG |
| | | CCGCCAAGUUCCGGGAGUACUUCCCCAACUUCGUGGGCGAGCCCA |
| | | AGUCCAAGGACAUCCUGAAGCUGCGGCUGUACGAGCAGCAGCACG |
| | | GCAAGUGCCUGUACUCCGGCAAGGAGAUCAACCUGGUGCGGCUG |
| | | AACGAGAAGGGCUACGUGGAGAUCGACCACGCCCUGCCCUUCUCC |
| | | CGGACCUGGGACGACUCCUUCAACAACAAGGUGCUGGUGCUGGGC |
| | | UCCGAGAACCAGAACAAGGGCAACCAGACCCCCUACGAGUACUUC |
| | | AACGGCAAGGACAACUCCCGGGAGUGGCAGGAGUUCAAGGCCCG |
| | | GGUGGAGACCUCCCGGUUCCCCCGGUCCAAGAAGCAGCGGAUCCU |
| | | GCUGCAGAAGUUCGACGAGGACGGCUUCAAGGAGUGCAACCUGA |
| | | ACGACACCCGGUACGUGAACCGCUUCCUGUGCCAGUUCGUGGCCG |
| | | ACCACAUCCUGCUGACCGGCAAGGGCAAGCGGCGGGUGUUCGCCU |
| | | CCAACGGCCAGAUCACCAACCUGCUGCGGGGCUUCUGGGGCCUGC |
| | | GGAAGGUGCGGGCCGAGAACGACCGGCACCACGCCCUGGACGCCG |
| | | UGGUGGUGGCCUGCUCCACCGUGGCCAUGCAGCAGAAGAUCACCC |
| | | GGUUCGUGCGGUACAAGGAGAUGAACGCCUUCGACGGCAAGACC |
| | | AUCGACAAGGAGACCGGCAAGGUGCUGCACCAGAAGACCCACUUC |
| | | CCCCAGCCCUGGGAGUUCUUCGCCCAGGAGGUGAUGAUCCGGGUG |
| | | UUCGGCAAGCCCGACGGCAAGCCCGAGUUCGAGGAGGCCGACACC |
| | | CCCGAGAAGCUGCGGACCCUGCUGGCCGAGAAGCUGUCCUCCCGG |
| | | CCCGAGGCCGUGCACGAGUACGUGACCCCCCUGUUCGUGUCCCGG |
| | | GCCCCCAACCGGAAGAUGUCCGGCGCCCACAAGGACACCCUGCGG |
| | | UCCGCCAAGCGGUUCGUGAAGCACAACGAGAAGAUCUCCGUGAA |
| | | GCGGGUGUGGCUGACCGAGAUCAAGCUGGCCGACCUGGAGAACA |
| | | UGGUGAACUACAAGAACGGCCGGGAGAUCGAGCUGUACGAGGCC |
| | | CUGAAGGCCCGGCUGGAGGCCUACGGCGGCAACGCCAAGCAGGCC |
| | | UUCGACCCCAAGGACAACCCCUUCUACAAGAAGGGCGGCCAGCUG |
| | | GUGAAGGCCGUGCGGGUGGAGAAGACCCAGGAGUCCGGCGUGCU |
| | | GCUGAACAAGAAGAACGCCUACACCAUCGCCGACAACGGCGACAU |
| | | GGUGCGGGUGGACGUGUUCUGCAAGGUGGACAAGAAGGGCAAGA |
| | | ACCAGUACUUCAUCGUGCCCAUCUACGCCUGGCAGGUGGCCGAGA |
| | | ACAUCCUGCCCGACAUCGACUGCAAGGGCUACCGGAUCGACGACU |
| | | CCUACACCUUCUGCUUCUCCCUGCACAAGUACGACCUGAUCGCCU |
| | | UCCAGAAGGACGAGAAGUCCAAGGUGGAGUUCGCCUACUACAUC |
| | | AACUGCGACUCCUCCAACGGCCGGUUCUACCUGGCCUGGCACGAC |
| | | AAGGGCUCCAAGGAGCAGCAGUUCCGGAUCUCCACCCAGAACCUG |
| | | GUGCUGAUCCAGAAGUACCAGGUGAACGAGCUGGGCAAGGAGAU |
| | | CCGGCCCUGCCGGCUGAAGAAGCGGCCCCCCGUGCGGUCCGGAAA |
| | | GCGGACCGCCGACGGCUCCGAGUUCGAGUCCCCCAAGAAGAAGCG |
| | | GAAGGUGGAGUAG |
| 136 | Exemplary amino | MEASPASGPRHLMDPHIFTSNFNNGIGRHKTYLCYEVERLDNGTSVKM |
| | acid sequence for | DQHRGFLHNQAKNLLCGFYGRHAELRFLDLVPSLQLDPAQIYRVTWFI |
| | APOBEC3A- | SWSPCFSWGCAGEVRAFLQENTHVRLRIFAARIYDYDPLYKEALQMLR |
| | Nme2D16A | DAGAQVSIMTYDEFKHCWDTFVDHQGCPFQPWDGLDEHSQALSGRL |
| | | RAILQNQGNSGSETPGTSESATPESAAFKPNPINYILGLAIGIASVGWAM |
| | | VEIDEEENPIRLIDLGVRVFERAEVPKTGDSLAMARRLARSVRRLTRRR |
| | | AHRLLRARRLLKREGVLQAADFDENGLIKSLPNTPWQLRAAALDRKL |
| | | TPLEWSAVLLHLIKHRGYLSQRKNEGETADKELGALLKGVANNAHAL |
| | | QTGDFRTPAELALNKFEKESGHIRNQRGDYSHTFSRKDLQAELILLFEK |
| | | QKEFGNPHVSGGLKEGIETLLMTQRPALSGDAVQKMLGHCTFEPAEPK |
| | | AAKNTYTAERFIWLTKLNNLRILEQGSERPLTDTERATLMDEPYRKSK |
| | | LTYAQARKLLGLEDTAFFKGLRYGKDNAEASTLMEMKAYHAISRALE |
| | | KEGLKDKKSPLNLSSELQDEIGTAFSLFKTDEDITGRLKDRVQPEILEAL |
| | | LKHISFDKFVQISLKALRRIVPLMEQGKRYDEACAEIYGDHYGKKNTE |
| | | EKIYLPPIPADEIRNPVVLRALSQARKVINGVVRRYGSPARIHIETAREV |
| | | GKSFKDRKEIEKRQEENRKDREKAAAKFREYFPNFVGEPKSKDILKLR |
| | | LYEQQHGKCLYSGKEINLVRLNEKGYVEIDHALPFSRTWDDSFNNKVL |
| | | VLGSENQNKGNQTPYEYFNGKDNSREWQEFKARVETSRFPRSKKQRIL |
| | | LQKFDEDGFKECNLNDTRYVNRFLCQFVADHILLTGKGKRRVFASNG |
| | | QITNLLRGFWGLRKVRAENDRHHALDAVVVACSTVAMQQKITRFVRY |
| | | KEMNAFDGKTIDKETGKVLHQKTHFPQPWEFFAQEVMIRVFGKPDGK |
| | | PEFEEADTPEKLRTLLAEKLSSRPEAVHEYVTPLFVSRAPNRKMSGAH |
| | | KDTLRSAKRFVKHNEKISVKRVWLTEIKLADLENMVNYKNGREIELYE |
| | | ALKARLEAYGGNAKQAFDPKDNPFYKKGGQLVKAVRVEKTQESGVL |
| | | LNKKNAYTIADNGDMVRVDVFCKVDKKGKNQYFIVPIYAWQVAENIL |
| | | PDIDCKGYRIDDSYTFCFSLHKYDLIAFQKDEKSKVEFAYYINCDSSNG |
| | | RFYLAWHDKGSKEQQFRISTQNLVLIQKYQVNELGKEIRPCRLKKRPP |
| | | VRSGKRTADGSEFESPKKKRKVE* |
| 137 | Exemplary | GGGAAGCUCAGAAUAAACGCUCAACUUUGGCCGGAUCUGCCACCA |
| | mRNA encoding | UGGACGGCUCCGGCGGCGGCUCCCCCAAGAAGAAGCGGAAGGUGG |
| | APOBEC3A- | AGGACAAGCGGCCCGCCGCCACCAAGAAGGCCGGCCAGGCCAAGA |
| | Nme2D16A | AGAAGAAGGGCGGCUCCGGCGGCGGCGAGGCCUCCCCCGCCUCCG |
| | | GCCCCCGGCACCUGAUGGACCCCCACAUCUUCACCUCCAACUUCA |
| | | ACAACGGCAUCGGCCGGCACAAGACCUACCUGUGCUACGAGGUGG |
| | | AGCGGCUGGACAACGGCACCUCCGUGAAGAUGGACCAGCACCGGG |
| | | GCUUCCUGCACAACCAGGCCAAGAACCUGCUGUGCGGCUUCUACG |
| | | GCCGGCACGCCGAGCUGCGGUUCCUGGACCUGGUGCCCUCCCUGC |
| | | AGCUGGACCCCGCCCAGAUCUACCGGGUGACCUGGUUCAUCUCCU |
| | | GGUCCCCCUGCUUCUCCUGGGGCUGCGCCGGCGAGGUGCGGGCCU |
| | | UCCUGCAGGAGAACACCCACGUGCGGCUGCGGAUCUUCGCCGCCC |
| | | GGAUCUACGACUACGACCCCCUGUACAAGGAGGCCCUGCAGAUGC |
| | | UGCGGGACGCCGGCGCCCAGGUGUCCAUCAUGACCUACGACGAGU |
| | | UCAAGCACUGCUGGGACACCUUCGUGGACCACCAGGGCUGCCCCU |
| | | UCCAGCCCUGGGACGGCCUGGACGAGCACUCCCAGGCCCUGUCCG |
| | | GCCGGCUGCGGGCCAUCCUGCAGAACCAGGGCAACUCCGGCUCCG |
| | | AGACCCCCGGCACCUCCGAGUCCGCCACCCCCGAGUCCGCAGCGU |
| | | UCAAACCAAAUCCCAUCAACUACAUCCUGGGCCUGGCCAUCGGCA |
| | | UCGCCUCCGUGGGCUGGGCCAUGGUGGAGAUCGACGAGGAGGAG |
| | | AACCCCAUCCGGCUGAUCGACCUGGGCGUGCGGGUGUUCGAGCGG |
| | | GCCGAGGUGCCCAAGACCGGCGACUCCCUGGCCAUGGCCCGGCGG |
| | | CUGGCCCGGUCCGUGCGGCGGCUGACCCGGCGGCGGGCCCACCGG |
| | | CUGCUGCGGGCCCGGCGGCUGCUGAAGCGGGAGGGCGUGCUGCAG |
| | | GCCGCCGACUUCGACGAGAACGGCCUGAUCAAGUCCCUGCCCAAC |
| | | ACCCCCUGGCAGCUGCGGGCCGCCGCCCUGGACCGGAAGCUGACC |
| | | CCCCUGGAGUGGUCCGCCGUGCUGCUGCACCUGAUCAAGCACCGG |
| | | GGCUACCUGUCCCAGCGGAAGAACGAGGGCGAGACCGCCGACAAG |
| | | GAGCUGGGCGCCCUGCUGAAGGGCGUGGCCAACAACGCCCACGCC |
| | | CUGCAGACCGGCGACUUCCGGACCCCCGCCGAGCUGGCCCUGAAC |
| | | AAGUUCGAGAAGGAGUCCGGCCACAUCCGGAACCAGCGGGGCGAC |
| | | UACUCCCACACCUUCUCCCGGAAGGACCUGCAGGCCGAGCUGAUC |
| | | CUGCUGUUCGAGAAGCAGAAGGAGUUCGGCAACCCCCACGUGUCC |
| | | GGCGGCCUGAAGGAGGGCAUCGAGACCCUGCUGAUGACCCAGCGG |
| | | CCCGCCCUGUCCGGCGACGCCGUGCAGAAGAUGCUGGGCCACUGC |
| | | ACCUUCGAGCCCGCCGAGCCCAAGGCCGCCAAGAACACCUACACC |
| | | GCCGAGCGGUUCAUCUGGCUGACCAAGCUGAACAACCUGCGGAUC |
| | | CUGGAGCAGGGCUCCGAGCGGCCCCUGACCGACACCGAGCGGGCC |
| | | ACCCUGAUGGACGAGCCCUACCGGAAGUCCAAGCUGACCUACGCC |
| | | CAGGCCCGGAAGCUGCUGGGCCUGGAGGACACCGCCUUCUUCAAG |
| | | GGCCUGCGGUACGGCAAGGACAACGCCGAGGCCUCCACCCUGAUG |
| | | GAGAUGAAGGCCUACCACGCCAUCUCCCGGGCCCUGGAGAAGGAG |
| | | GGCCUGAAGGACAAGAAGUCCCCCCUGAACCUGUCCUCCGAGCUG |
| | | CAGGACGAGAUCGGCACCGCCUUCUCCCUGUUCAAGACCGACGAG |
| | | GACAUCACCGGCCGGCUGAAGGACCGGGUGCAGCCCGAGAUCCUG |
| | | GAGGCCCUGCUGAAGCACAUCUCCUUCGACAAGUUCGUGCAGAUC |
| | | UCCCUGAAGGCCCUGCGGCGGAUCGUGCCCCUGAUGGAGCAGGGC |
| | | AAGCGGUACGACGAGGCCUGCGCCGAGAUCUACGGCGACCACUAC |
| | | GGCAAGAAGAACACCGAGGAGAAGAUCUACCUGCCCCCCAUCCCC |
| | | GCCGACGAGAUCCGGAACCCCGUGGUGCUGCGGGCCCUGUCCCAG |
| | | GCCCGGAAGGUGAUCAACGGCGUGGUGCGGCGGUACGGCUCCCCC |
| | | GCCCGGAUCCACAUCGAGACCGCCCGGGAGGUGGGCAAGUCCUUC |
| | | AAGGACCGGAAGGAGAUCGAGAAGCGGCAGGAGGAGAACCGGAA |
| | | GGACCGGGAGAAGGCCGCCGCCAAGUUCCGGGAGUACUUCCCCAA |
| | | CUUCGUGGGCGAGCCCAAGUCCAAGGACAUCCUGAAGCUGCGGCU |
| | | GUACGAGCAGCAGCACGGCAAGUGCCUGUACUCCGGCAAGGAGA |
| | | UCAACCUGGUGCGGCUGAACGAGAAGGGCUACGUGGAGAUCGAC |
| | | CACGCCCUGCCCUUCUCCCGGACCUGGGACGACUCCUUCAACAAC |
| | | AAGGUGCUGGUGCUGGGCUCCGAGAACCAGAACAAGGGCAACCA |
| | | GACCCCCUACGAGUACUUCAACGGCAAGGACAACUCCCGGGAGUG |
| | | GCAGGAGUUCAAGGCCCGGGUGGAGACCUCCCGGUUCCCCCGGUC |
| | | CAAGAAGCAGCGGAUCCUGCUGCAGAAGUUCGACGAGGACGGCU |
| | | UCAAGGAGUGCAACCUGAACGACACCCGGUACGUGAACCGCUUCC |
| | | UGUGCCAGUUCGUGGCCGACCACAUCCUGCUGACCGGCAAGGGCA |
| | | AGCGGCGGGUGUUCGCCUCCAACGGCCAGAUCACCAACCUGCUGC |
| | | GGGGCUUCUGGGGCCUGCGGAAGGUGCGGGCCGAGAACGACCGG |
| | | CACCACGCCCUGGACGCCGUGGUGGUGGCCUGCUCCACCGUGGCC |
| | | AUGCAGCAGAAGAUCACCCGGUUCGUGCGGUACAAGGAGAUGAA |
| | | CGCCUUCGACGGCAAGACCAUCGACAAGGAGACCGGCAAGGUGCU |
| | | GCACCAGAAGACCCACUUCCCCCAGCCCUGGGAGUUCUUCGCCCA |
| | | GGAGGUGAUGAUCCGGGUGUUCGGCAAGCCCGACGGCAAGCCCG |
| | | AGUUCGAGGAGGCCGACACCCCCGAGAAGCUGCGGACCCUGCUGG |
| | | CCGAGAAGCUGUCCUCCCGGCCCGAGGCCGUGCACGAGUACGUGA |
| | | CCCCCCUGUUCGUGUCCCGGGCCCCCAACCGGAAGAUGUCCGGCG |
| | | CCCACAAGGACACCCUGCGGUCCGCCAAGCGGUUCGUGAAGCACA |
| | | ACGAGAAGAUCUCCGUGAAGCGGGUGUGGCUGACCGAGAUCAAG |
| | | CUGGCCGACCUGGAGAACAUGGUGAACUACAAGAACGGCCGGGA |
| | | GAUCGAGCUGUACGAGGCCCUGAAGGCCCGGCUGGAGGCCUACGG |
| | | CGGCAACGCCAAGCAGGCCUUCGACCCCAAGGACAACCCCUUCUA |
| | | CAAGAAGGGCGGCCAGCUGGUGAAGGCCGUGCGGGUGGAGAAGA |
| | | CCCAGGAGUCCGGCGUGCUGCUGAACAAGAAGAACGCCUACACCA |
| | | UCGCCGACAACGGCGACAUGGUGCGGGUGGACGUGUUCUGCAAG |
| | | GUGGACAAGAAGGGCAAGAACCAGUACUUCAUCGUGCCCAUCUA |
| | | CGCCUGGCAGGUGGCCGAGAACAUCCUGCCCGACAUCGACUGCAA |
| | | GGGCUACCGGAUCGACGACUCCUACACCUUCUGCUUCUCCCUGCA |
| | | CAAGUACGACCUGAUCGCCUUCCAGAAGGACGAGAAGUCCAAGG |
| | | UGGAGUUCGCCUACUACAUCAACUGCGACUCCUCCAACGGCCGGU |
| | | UCUACCUGGCCUGGCACGACAAGGGCUCCAAGGAGCAGCAGUUCC |
| | | GGAUCUCCACCCAGAACCUGGUGCUGAUCCAGAAGUACCAGGUGA |
| | | ACGAGCUGGGCAAGGAGAUCCGGCCCUGCCGGCUGAAGAAGCGGC |
| | | CCCCCGUGCGGUCCGGAAAGCGGACCGCCGACGGCUCCGAGUUCG |
| | | AGUCCCCCAAGAAGAAGCGGAAGGUGGAGUAGUGACUAGCACCA |
| | | GCCUCAAGAACACCCGAAUGGAGUCUCUAAGCUACAUAAUACCAA |
| | | CUUACACUUUACAAAAUGUUGUCCCCCAAAAUGUAGCCAUUCGU |
| | | AUCUGCUCCUAAUAAAAAGAAAGUUUCUUCACAUUCU |
| 138 | Exemplary open | AUGGACGGCUCCGGCGGCGGCUCCCCCAAGAAGAAGCGGAAGGUG |
| | reading frame for | GAGGACAAGCGGCCCGCCGCCACCAAGAAGGCCGGCCAGGCCAAG |
| | APOBEC3A- | AAGAAGAAGGGCGGCUCCGGCGGCGGCGAGGCCUCCCCCGCCUCC |
| | Nme2D16A | GGCCCCCGGCACCUGAUGGACCCCCACAUCUUCACCUCCAACUUC |
| | | AACAACGGCAUCGGCCGGCACAAGACCUACCUGUGCUACGAGGUG |
| | | GAGCGGCUGGACAACGGCACCUCCGUGAAGAUGGACCAGCACCGG |
| | | GGCUUCCUGCACAACCAGGCCAAGAACCUGCUGUGCGGCUUCUAC |
| | | GGCCGGCACGCCGAGCUGCGGUUCCUGGACCUGGUGCCCUCCCUG |
| | | CAGCUGGACCCCGCCCAGAUCUACCGGGUGACCUGGUUCAUCUCC |
| | | UGGUCCCCCUGCUUCUCCUGGGGCUGCGCCGGCGAGGUGCGGGCC |
| | | UUCCUGCAGGAGAACACCCACGUGCGGCUGCGGAUCUUCGCCGCC |
| | | CGGAUCUACGACUACGACCCCCUGUACAAGGAGGCCCUGCAGAUG |
| | | CUGCGGGACGCCGGCGCCCAGGUGUCCAUCAUGACCUACGACGAG |
| | | UUCAAGCACUGCUGGGACACCUUCGUGGACCACCAGGGCUGCCCC |
| | | UUCCAGCCCUGGGACGGCCUGGACGAGCACUCCCAGGCCCUGUCC |
| | | GGCCGGCUGCGGGCCAUCCUGCAGAACCAGGGCAACUCCGGCUCC |
| | | GAGACCCCCGGCACCUCCGAGUCCGCCACCCCCGAGUCCGCAGCG |
| | | UUCAAACCAAAUCCCAUCAACUACAUCCUGGGCCUGGCCAUCGGC |
| | | AUCGCCUCCGUGGGCUGGGCCAUGGUGGAGAUCGACGAGGAGGA |
| | | GAACCCCAUCCGGCUGAUCGACCUGGGCGUGCGGGUGUUCGAGCG |
| | | GGCCGAGGUGCCCAAGACCGGCGACUCCCUGGCCAUGGCCCGGCG |
| | | GCUGGCCCGGUCCGUGCGGCGGCUGACCCGGCGGCGGGCCCACCG |
| | | GCUGCUGCGGGCCCGGCGGCUGCUGAAGCGGGAGGGCGUGCUGCA |
| | | GGCCGCCGACUUCGACGAGAACGGCCUGAUCAAGUCCCUGCCCAA |
| | | CACCCCCUGGCAGCUGCGGGCCGCCGCCCUGGACCGGAAGCUGAC |
| | | CCCCCUGGAGUGGUCCGCCGUGCUGCUGCACCUGAUCAAGCACCG |
| | | GGGCUACCUGUCCCAGCGGAAGAACGAGGGCGAGACCGCCGACAA |
| | | GGAGCUGGGCGCCCUGCUGAAGGGCGUGGCCAACAACGCCCACGC |
| | | CCUGCAGACCGGCGACUUCCGGACCCCCGCCGAGCUGGCCCUGAA |
| | | CAAGUUCGAGAAGGAGUCCGGCCACAUCCGGAACCAGCGGGGCGA |
| | | CUACUCCCACACCUUCUCCCGGAAGGACCUGCAGGCCGAGCUGAU |
| | | CCUGCUGUUCGAGAAGCAGAAGGAGUUCGGCAACCCCCACGUGUC |
| | | CGGCGGCCUGAAGGAGGGCAUCGAGACCCUGCUGAUGACCCAGCG |
| | | GCCCGCCCUGUCCGGCGACGCCGUGCAGAAGAUGCUGGGCCACUG |
| | | CACCUUCGAGCCCGCCGAGCCCAAGGCCGCCAAGAACACCUACAC |
| | | CGCCGAGCGGUUCAUCUGGCUGACCAAGCUGAACAACCUGCGGAU |
| | | CCUGGAGCAGGGCUCCGAGCGGCCCCUGACCGACACCGAGCGGGC |
| | | CACCCUGAUGGACGAGCCCUACCGGAAGUCCAAGCUGACCUACGC |
| | | CCAGGCCCGGAAGCUGCUGGGCCUGGAGGACACCGCCUUCUUCAA |
| | | GGGCCUGCGGUACGGCAAGGACAACGCCGAGGCCUCCACCCUGAU |
| | | GGAGAUGAAGGCCUACCACGCCAUCUCCCGGGCCCUGGAGAAGGA |
| | | GGGCCUGAAGGACAAGAAGUCCCCCCUGAACCUGUCCUCCGAGCU |
| | | GCAGGACGAGAUCGGCACCGCCUUCUCCCUGUUCAAGACCGACGA |
| | | GGACAUCACCGGCCGGCUGAAGGACCGGGUGCAGCCCGAGAUCCU |
| | | GGAGGCCCUGCUGAAGCACAUCUCCUUCGACAAGUUCGUGCAGAU |
| | | CUCCCUGAAGGCCCUGCGGCGGAUCGUGCCCCUGAUGGAGCAGGG |
| | | CAAGCGGUACGACGAGGCCUGCGCCGAGAUCUACGGCGACCACUA |
| | | CGGCAAGAAGAACACCGAGGAGAAGAUCUACCUGCCCCCCAUCCC |
| | | CGCCGACGAGAUCCGGAACCCCGUGGUGCUGCGGGCCCUGUCCCA |
| | | GGCCCGGAAGGUGAUCAACGGCGUGGUGCGGCGGUACGGCUCCCC |
| | | CGCCCGGAUCCACAUCGAGACCGCCCGGGAGGUGGGCAAGUCCUU |
| | | CAAGGACCGGAAGGAGAUCGAGAAGCGGCAGGAGGAGAACCGGA |
| | | AGGACCGGGAGAAGGCCGCCGCCAAGUUCCGGGAGUACUUCCCCA |
| | | ACUUCGUGGGCGAGCCCAAGUCCAAGGACAUCCUGAAGCUGCGGC |
| | | UGUACGAGCAGCAGCACGGCAAGUGCCUGUACUCCGGCAAGGAG |
| | | AUCAACCUGGUGCGGCUGAACGAGAAGGGCUACGUGGAGAUCGA |
| | | CCACGCCCUGCCCUUCUCCCGGACCUGGGACGACUCCUUCAACAA |
| | | CAAGGUGCUGGUGCUGGGCUCCGAGAACCAGAACAAGGGCAACC |
| | | AGACCCCCUACGAGUACUUCAACGGCAAGGACAACUCCCGGGAGU |
| | | GGCAGGAGUUCAAGGCCCGGGUGGAGACCUCCCGGUUCCCCCGGU |
| | | CCAAGAAGCAGCGGAUCCUGCUGCAGAAGUUCGACGAGGACGGC |
| | | UUCAAGGAGUGCAACCUGAACGACACCCGGUACGUGAACCGCUUC |
| | | CUGUGCCAGUUCGUGGCCGACCACAUCCUGCUGACCGGCAAGGGC |
| | | AAGCGGCGGGUGUUCGCCUCCAACGGCCAGAUCACCAACCUGCUG |
| | | CGGGGCUUCUGGGGCCUGCGGAAGGUGCGGGCCGAGAACGACCG |
| | | GCACCACGCCCUGGACGCCGUGGUGGUGGCCUGCUCCACCGUGGC |
| | | CAUGCAGCAGAAGAUCACCCGGUUCGUGCGGUACAAGGAGAUGA |
| | | ACGCCUUCGACGGCAAGACCAUCGACAAGGAGACCGGCAAGGUGC |
| | | UGCACCAGAAGACCCACUUCCCCCAGCCCUGGGAGUUCUUCGCCC |
| | | AGGAGGUGAUGAUCCGGGUGUUCGGCAAGCCCGACGGCAAGCCC |
| | | GAGUUCGAGGAGGCCGACACCCCCGAGAAGCUGCGGACCCUGCUG |
| | | GCCGAGAAGCUGUCCUCCCGGCCCGAGGCCGUGCACGAGUACGUG |
| | | ACCCCCCUGUUCGUGUCCCGGGCCCCCAACCGGAAGAUGUCCGGC |
| | | GCCCACAAGGACACCCUGCGGUCCGCCAAGCGGUUCGUGAAGCAC |
| | | AACGAGAAGAUCUCCGUGAAGCGGGUGUGGCUGACCGAGAUCAA |
| | | GCUGGCCGACCUGGAGAACAUGGUGAACUACAAGAACGGCCGGG |
| | | AGAUCGAGCUGUACGAGGCCCUGAAGGCCCGGCUGGAGGCCUACG |
| | | GCGGCAACGCCAAGCAGGCCUUCGACCCCAAGGACAACCCCUUCU |
| | | ACAAGAAGGGCGGCCAGCUGGUGAAGGCCGUGCGGGUGGAGAAG |
| | | ACCCAGGAGUCCGGCGUGCUGCUGAACAAGAAGAACGCCUACACC |
| | | AUCGCCGACAACGGCGACAUGGUGCGGGUGGACGUGUUCUGCAA |
| | | GGUGGACAAGAAGGGCAAGAACCAGUACUUCAUCGUGCCCAUCU |
| | | ACGCCUGGCAGGUGGCCGAGAACAUCCUGCCCGACAUCGACUGCA |
| | | AGGGCUACCGGAUCGACGACUCCUACACCUUCUGCUUCUCCCUGC |
| | | ACAAGUACGACCUGAUCGCCUUCCAGAAGGACGAGAAGUCCAAG |
| | | GUGGAGUUCGCCUACUACAUCAACUGCGACUCCUCCAACGGCCGG |
| | | UUCUACCUGGCCUGGCACGACAAGGGCUCCAAGGAGCAGCAGUUC |
| | | CGGAUCUCCACCCAGAACCUGGUGCUGAUCCAGAAGUACCAGGUG |
| | | AACGAGCUGGGCAAGGAGAUCCGGCCCUGCCGGCUGAAGAAGCG |
| | | GCCCCCCGUGCGGUCCGGAAAGCGGACCGCCGACGGCUCCGAGUU |
| | | CGAGUCCCCCAAGAAGAAGCGGAAGGUGGAGUAG |
| 139 | Exemplary amino | MDGSGGGSPKKKRKVEDKRPAATKKAGQAKKKKGGSGGGEASPASG |
| | acid sequence for | PRHLMDPHIFTSNFNNGIGRHKTYLCYEVERLDNGTSVKMDQHRGFLH |
| | APOBEC3A- | NQAKNLLCGFYGRHAELRFLDLVPSLQLDPAQIYRVTWFISWSPCFSW |
| | Nme2D16A | GCAGEVRAFLQENTHVRLRIFAARIYDYDPLYKEALQMLRDAGAQVSI |
| | | MTYDEFKHCWDTFVDHQGCPFQPWDGLDEHSQALSGRLRAILQNQG |
| | | NSGSETPGTSESATPESAAFKPNPINYILGLAIGIASVGWAMVEIDEEEN |
| | | PIRLIDLGVRVFERAEVPKTGDSLAMARRLARSVRRLTRRRAHRLLRA |
| | | RRLLKREGVLQAADFDENGLIKSLPNTPWQLRAAALDRKLTPLEWSA |
| | | VLLHLIKHRGYLSQRKNEGETADKELGALLKGVANNAHALQTGDFRT |
| | | PAELALNKFEKESGHIRNQRGDYSHTFSRKDLQAELILLFEKQKEFGNP |
| | | HVSGGLKEGIETLLMTQRPALSGDAVQKMLGHCTFEPAEPKAAKNTY |
| | | TAERFIWLTKLNNLRILEQGSERPLTDTERATLMDEPYRKSKLTYAQA |
| | | RKLLGLEDTAFFKGLRYGKDNAEASTLMEMKAYHAISRALEKEGLKD |
| | | KKSPLNLSSELQDEIGTAFSLFKTDEDITGRLKDRVQPEILEALLKHISFD |
| | | KFVQISLKALRRIVPLMEQGKRYDEACAEIYGDHYGKKNTEEKIYLPPI |
| | | PADEIRNPVVLRALSQARKVINGVVRRYGSPARIHIETAREVGKSFKDR |
| | | KEIEKRQEENRKDREKAAAKFREYFPNFVGEPKSKDILKLRLYEQQHG |
| | | KCLYSGKEINLVRLNEKGYVEIDHALPFSRTWDDSFNNKVLVLGSENQ |
| | | NKGNQTPYEYFNGKDNSREWQEFKARVETSRFPRSKKQRILLQKFDED |
| | | GFKECNLNDTRYVNRFLCQFVADHILLTGKGKRRVFASNGQITNLLRG |
| | | FWGLRKVRAENDRHHALDAVVVACSTVAMQQKITRFVRYKEMNAFD |
| | | GKTIDKETGKVLHQKTHFPQPWEFFAQEVMIRVFGKPDGKPEFEEADT |
| | | PEKLRTLLAEKLSSRPEAVHEYVTPLFVSRAPNRKMSGAHKDTLRSAK |
| | | RFVKHNEKISVKRVWLTEIKLADLENMVNYKNGREIELYEALKARLEA |
| | | YGGNAKQAFDPKDNPFYKKGGQLVKAVRVEKTQESGVLLNKKNAYT |
| | | IADNGDMVRVDVFCKVDKKGKNQYFIVPIYAWQVAENILPDIDCKGY |
| | | RIDDSYTFCFSLHKYDLIAFQKDEKSKVEFAYYINCDSSNGRFYLAWH |
| | | DKGSKEQQFRISTQNLVLIQKYQVNELGKEIRPCRLKKRPPVRSGKRTA |
| | | DGSEFESPKKKRKVE* |
| 140 | Exemplary | GGGAAGCUCAGAAUAAACGCUCAACUUUGGCCGGAUCUGCCACCA |
| | mRNA encoding | UGGACGGCUCCGGCGGCGGCUCCCCCAAGAAGAAGCGGAAGGUGG |
| | APOBEC3A- | AGGACAAGCGGCCCGCCGCCACCAAGAAGGCCGGCCAGGCCAAGA |
| | Nme2D16A | AGAAGAAGGGCGGCUCCGGCGGCGGCGAGGCCUCCCCCGCCUCCG |
| | | GCCCCCGGCACCUGAUGGACCCCCACAUCUUCACCUCCAACUUCA |
| | | ACAACGGCAUCGGCCGGCACAAGACCUACCUGUGCUACGAGGUGG |
| | | AGCGGCUGGACAACGGCACCUCCGUGAAGAUGGACCAGCACCGGG |
| | | GCUUCCUGCACAACCAGGCCAAGAACCUGCUGUGCGGCUUCUACG |
| | | GCCGGCACGCCGAGCUGCGGUUCCUGGACCUGGUGCCCUCCCUGC |
| | | AGCUGGACCCCGCCCAGAUCUACCGGGUGACCUGGUUCAUCUCCU |
| | | GGUCCCCCUGCUUCUCCUGGGGCUGCGCCGGCGAGGUGCGGGCCU |
| | | UCCUGCAGGAGAACACCCACGUGCGGCUGCGGAUCUUCGCCGCCC |
| | | GGAUCUACGACUACGACCCCCUGUACAAGGAGGCCCUGCAGAUGC |
| | | UGCGGGACGCCGGCGCCCAGGUGUCCAUCAUGACCUACGACGAGU |
| | | UCAAGCACUGCUGGGACACCUUCGUGGACCACCAGGGCUGCCCCU |
| | | UCCAGCCCUGGGACGGCCUGGACGAGCACUCCCAGGCCCUGUCCG |
| | | GCCGGCUGCGGGCCAUCCUGCAGAACCAGGGCAACUCCGGCUCCG |
| | | AGACCCCCGGCACCUCCGAGUCCGCCACCCCCGAGUCCGCAGCGU |
| | | UCAAACCAAAUCCCAUCAACUACAUCCUGGGCCUGGCCAUCGGCA |
| | | UCGCCUCCGUGGGCUGGGCCAUGGUGGAGAUCGACGAGGAGGAG |
| | | AACCCCAUCCGGCUGAUCGACCUGGGCGUGCGGGUGUUCGAGCGG |
| | | GCCGAGGUGCCCAAGACCGGCGACUCCCUGGCCAUGGCCCGGCGG |
| | | CUGGCCCGGUCCGUGCGGCGGCUGACCCGGCGGCGGGCCCACCGG |
| | | CUGCUGCGGGCCCGGCGGCUGCUGAAGCGGGAGGGCGUGCUGCAG |
| | | GCCGCCGACUUCGACGAGAACGGCCUGAUCAAGUCCCUGCCCAAC |
| | | ACCCCCUGGCAGCUGCGGGCCGCCGCCCUGGACCGGAAGCUGACC |
| | | CCCCUGGAGUGGUCCGCCGUGCUGCUGCACCUGAUCAAGCACCGG |
| | | GGCUACCUGUCCCAGCGGAAGAACGAGGGCGAGACCGCCGACAAG |
| | | GAGCUGGGCGCCCUGCUGAAGGGCGUGGCCAACAACGCCCACGCC |
| | | CUGCAGACCGGCGACUUCCGGACCCCCGCCGAGCUGGCCCUGAAC |
| | | AAGUUCGAGAAGGAGUCCGGCCACAUCCGGAACCAGCGGGGCGAC |
| | | UACUCCCACACCUUCUCCCGGAAGGACCUGCAGGCCGAGCUGAUC |
| | | CUGCUGUUCGAGAAGCAGAAGGAGUUCGGCAACCCCCACGUGUCC |
| | | GGCGGCCUGAAGGAGGGCAUCGAGACCCUGCUGAUGACCCAGCGG |
| | | CCCGCCCUGUCCGGCGACGCCGUGCAGAAGAUGCUGGGCCACUGC |
| | | ACCUUCGAGCCCGCCGAGCCCAAGGCCGCCAAGAACACCUACACC |
| | | GCCGAGCGGUUCAUCUGGCUGACCAAGCUGAACAACCUGCGGAUC |
| | | CUGGAGCAGGGCUCCGAGCGGCCCCUGACCGACACCGAGCGGGCC |
| | | ACCCUGAUGGACGAGCCCUACCGGAAGUCCAAGCUGACCUACGCC |
| | | CAGGCCCGGAAGCUGCUGGGCCUGGAGGACACCGCCUUCUUCAAG |
| | | GGCCUGCGGUACGGCAAGGACAACGCCGAGGCCUCCACCCUGAUG |
| | | GAGAUGAAGGCCUACCACGCCAUCUCCCGGGCCCUGGAGAAGGAG |
| | | GGCCUGAAGGACAAGAAGUCCCCCCUGAACCUGUCCUCCGAGCUG |
| | | CAGGACGAGAUCGGCACCGCCUUCUCCCUGUUCAAGACCGACGAG |
| | | GACAUCACCGGCCGGCUGAAGGACCGGGUGCAGCCCGAGAUCCUG |
| | | GAGGCCCUGCUGAAGCACAUCUCCUUCGACAAGUUCGUGCAGAUC |
| | | UCCCUGAAGGCCCUGCGGCGGAUCGUGCCCCUGAUGGAGCAGGGC |
| | | AAGCGGUACGACGAGGCCUGCGCCGAGAUCUACGGCGACCACUAC |
| | | GGCAAGAAGAACACCGAGGAGAAGAUCUACCUGCCCCCCAUCCCC |
| | | GCCGACGAGAUCCGGAACCCCGUGGUGCUGCGGGCCCUGUCCCAG |
| | | GCCCGGAAGGUGAUCAACGGCGUGGUGCGGCGGUACGGCUCCCCC |
| | | GCCCGGAUCCACAUCGAGACCGCCCGGGAGGUGGGCAAGUCCUUC |
| | | AAGGACCGGAAGGAGAUCGAGAAGCGGCAGGAGGAGAACCGGAA |
| | | GGACCGGGAGAAGGCCGCCGCCAAGUUCCGGGAGUACUUCCCCAA |
| | | CUUCGUGGGCGAGCCCAAGUCCAAGGACAUCCUGAAGCUGCGGCU |
| | | GUACGAGCAGCAGCACGGCAAGUGCCUGUACUCCGGCAAGGAGA |
| | | UCAACCUGGUGCGGCUGAACGAGAAGGGCUACGUGGAGAUCGAC |
| | | CACGCCCUGCCCUUCUCCCGGACCUGGGACGACUCCUUCAACAAC |
| | | AAGGUGCUGGUGCUGGGCUCCGAGAACCAGAACAAGGGCAACCA |
| | | GACCCCCUACGAGUACUUCAACGGCAAGGACAACUCCCGGGAGUG |
| | | GCAGGAGUUCAAGGCCCGGGUGGAGACCUCCCGGUUCCCCCGGUC |
| | | CAAGAAGCAGCGGAUCCUGCUGCAGAAGUUCGACGAGGACGGCU |
| | | UCAAGGAGUGCAACCUGAACGACACCCGGUACGUGAACCGCUUCC |
| | | UGUGCCAGUUCGUGGCCGACCACAUCCUGCUGACCGGCAAGGGCA |
| | | AGCGGCGGGUGUUCGCCUCCAACGGCCAGAUCACCAACCUGCUGC |
| | | GGGGCUUCUGGGGCCUGCGGAAGGUGCGGGCCGAGAACGACCGG |
| | | CACCACGCCCUGGACGCCGUGGUGGUGGCCUGCUCCACCGUGGCC |
| | | AUGCAGCAGAAGAUCACCCGGUUCGUGCGGUACAAGGAGAUGAA |
| | | CGCCUUCGACGGCAAGACCAUCGACAAGGAGACCGGCAAGGUGCU |
| | | GCACCAGAAGACCCACUUCCCCCAGCCCUGGGAGUUCUUCGCCCA |
| | | GGAGGUGAUGAUCCGGGUGUUCGGCAAGCCCGACGGCAAGCCCG |
| | | AGUUCGAGGAGGCCGACACCCCCGAGAAGCUGCGGACCCUGCUGG |
| | | CCGAGAAGCUGUCCUCCCGGCCCGAGGCCGUGCACGAGUACGUGA |
| | | CCCCCCUGUUCGUGUCCCGGGCCCCCAACCGGAAGAUGUCCGGCG |
| | | CCCACAAGGACACCCUGCGGUCCGCCAAGCGGUUCGUGAAGCACA |
| | | ACGAGAAGAUCUCCGUGAAGCGGGUGUGGCUGACCGAGAUCAAG |
| | | CUGGCCGACCUGGAGAACAUGGUGAACUACAAGAACGGCCGGGA |
| | | GAUCGAGCUGUACGAGGCCCUGAAGGCCCGGCUGGAGGCCUACGG |
| | | CGGCAACGCCAAGCAGGCCUUCGACCCCAAGGACAACCCCUUCUA |
| | | CAAGAAGGGCGGCCAGCUGGUGAAGGCCGUGCGGGUGGAGAAGA |
| | | CCCAGGAGUCCGGCGUGCUGCUGAACAAGAAGAACGCCUACACCA |
| | | UCGCCGACAACGGCGACAUGGUGCGGGUGGACGUGUUCUGCAAG |
| | | GUGGACAAGAAGGGCAAGAACCAGUACUUCAUCGUGCCCAUCUA |
| | | CGCCUGGCAGGUGGCCGAGAACAUCCUGCCCGACAUCGACUGCAA |
| | | GGGCUACCGGAUCGACGACUCCUACACCUUCUGCUUCUCCCUGCA |
| | | CAAGUACGACCUGAUCGCCUUCCAGAAGGACGAGAAGUCCAAGG |
| | | UGGAGUUCGCCUACUACAUCAACUGCGACUCCUCCAACGGCCGGU |
| | | UCUACCUGGCCUGGCACGACAAGGGCUCCAAGGAGCAGCAGUUCC |
| | | GGAUCUCCACCCAGAACCUGGUGCUGAUCCAGAAGUACCAGGUGA |
| | | ACGAGCUGGGCAAGGAGAUCCGGCCCUGCCGGCUGAAGAAGCGGC |
| | | CCCCCGUGCGGGAGGACAAGCGGCCCGCCGCCACCAAGAAGGCCG |
| | | GCCAGGCCAAGAAGAAGAAGUACCCCUACGACGUGCCCGACUACG |
| | | CCGGCUACCCCUACGACGUGCCCGACUACGCCGGCUCCUACCCCU |
| | | ACGACGUGCCCGACUACGCCGCCGCCCCCGCCGCCAAGAAGAAGA |
| | | AGCUGGACUAGCUAGUGACUAGCACCAGCCUCAAGAACACCCGAA |
| | | UGGAGUCUCUAAGCUACAUAAUACCAACUUACACUUUACAAAAU |
| | | GUUGUCCCCCAAAAUGUAGCCAUUCGUAUCUGCUCCUAAUAAAA |
| | | AGAAAGUUUCUUCACAUUCU |
| 141 | Exemplary open | AUGGACGGCUCCGGCGGCGGCUCCCCCAAGAAGAAGCGGAAGGUG |
| | reading frame for | GAGGACAAGCGGCCCGCCGCCACCAAGAAGGCCGGCCAGGCCAAG |
| | APOBEC3A- | AAGAAGAAGGGCGGCUCCGGCGGCGGCGAGGCCUCCCCCGCCUCC |
| | Nme2D16A | GGCCCCCGGCACCUGAUGGACCCCCACAUCUUCACCUCCAACUUC |
| | | AACAACGGCAUCGGCCGGCACAAGACCUACCUGUGCUACGAGGUG |
| | | GAGCGGCUGGACAACGGCACCUCCGUGAAGAUGGACCAGCACCGG |
| | | GGCUUCCUGCACAACCAGGCCAAGAACCUGCUGUGCGGCUUCUAC |
| | | GGCCGGCACGCCGAGCUGCGGUUCCUGGACCUGGUGCCCUCCCUG |
| | | CAGCUGGACCCCGCCCAGAUCUACCGGGUGACCUGGUUCAUCUCC |
| | | UGGUCCCCCUGCUUCUCCUGGGGCUGCGCCGGCGAGGUGCGGGCC |
| | | UUCCUGCAGGAGAACACCCACGUGCGGCUGCGGAUCUUCGCCGCC |
| | | CGGAUCUACGACUACGACCCCCUGUACAAGGAGGCCCUGCAGAUG |
| | | CUGCGGGACGCCGGCGCCCAGGUGUCCAUCAUGACCUACGACGAG |
| | | UUCAAGCACUGCUGGGACACCUUCGUGGACCACCAGGGCUGCCCC |
| | | UUCCAGCCCUGGGACGGCCUGGACGAGCACUCCCAGGCCCUGUCC |
| | | GGCCGGCUGCGGGCCAUCCUGCAGAACCAGGGCAACUCCGGCUCC |
| | | GAGACCCCCGGCACCUCCGAGUCCGCCACCCCCGAGUCCGCAGCG |
| | | UUCAAACCAAAUCCCAUCAACUACAUCCUGGGCCUGGCCAUCGGC |
| | | AUCGCCUCCGUGGGCUGGGCCAUGGUGGAGAUCGACGAGGAGGA |
| | | GAACCCCAUCCGGCUGAUCGACCUGGGCGUGCGGGUGUUCGAGCG |
| | | GGCCGAGGUGCCCAAGACCGGCGACUCCCUGGCCAUGGCCCGGCG |
| | | GCUGGCCCGGUCCGUGCGGCGGCUGACCCGGCGGCGGGCCCACCG |
| | | GCUGCUGCGGGCCCGGCGGCUGCUGAAGCGGGAGGGCGUGCUGCA |
| | | GGCCGCCGACUUCGACGAGAACGGCCUGAUCAAGUCCCUGCCCAA |
| | | CACCCCCUGGCAGCUGCGGGCCGCCGCCCUGGACCGGAAGCUGAC |
| | | CCCCCUGGAGUGGUCCGCCGUGCUGCUGCACCUGAUCAAGCACCG |
| | | GGGCUACCUGUCCCAGCGGAAGAACGAGGGCGAGACCGCCGACAA |
| | | GGAGCUGGGCGCCCUGCUGAAGGGCGUGGCCAACAACGCCCACGC |
| | | CCUGCAGACCGGCGACUUCCGGACCCCCGCCGAGCUGGCCCUGAA |
| | | CAAGUUCGAGAAGGAGUCCGGCCACAUCCGGAACCAGCGGGGCGA |
| | | CUACUCCCACACCUUCUCCCGGAAGGACCUGCAGGCCGAGCUGAU |
| | | CCUGCUGUUCGAGAAGCAGAAGGAGUUCGGCAACCCCCACGUGUC |
| | | CGGCGGCCUGAAGGAGGGCAUCGAGACCCUGCUGAUGACCCAGCG |
| | | GCCCGCCCUGUCCGGCGACGCCGUGCAGAAGAUGCUGGGCCACUG |
| | | CACCUUCGAGCCCGCCGAGCCCAAGGCCGCCAAGAACACCUACAC |
| | | CGCCGAGCGGUUCAUCUGGCUGACCAAGCUGAACAACCUGCGGAU |
| | | CCUGGAGCAGGGCUCCGAGCGGCCCCUGACCGACACCGAGCGGGC |
| | | CACCCUGAUGGACGAGCCCUACCGGAAGUCCAAGCUGACCUACGC |
| | | CCAGGCCCGGAAGCUGCUGGGCCUGGAGGACACCGCCUUCUUCAA |
| | | GGGCCUGCGGUACGGCAAGGACAACGCCGAGGCCUCCACCCUGAU |
| | | GGAGAUGAAGGCCUACCACGCCAUCUCCCGGGCCCUGGAGAAGGA |
| | | GGGCCUGAAGGACAAGAAGUCCCCCCUGAACCUGUCCUCCGAGCU |
| | | GCAGGACGAGAUCGGCACCGCCUUCUCCCUGUUCAAGACCGACGA |
| | | GGACAUCACCGGCCGGCUGAAGGACCGGGUGCAGCCCGAGAUCCU |
| | | GGAGGCCCUGCUGAAGCACAUCUCCUUCGACAAGUUCGUGCAGAU |
| | | CUCCCUGAAGGCCCUGCGGCGGAUCGUGCCCCUGAUGGAGCAGGG |
| | | CAAGCGGUACGACGAGGCCUGCGCCGAGAUCUACGGCGACCACUA |
| | | CGGCAAGAAGAACACCGAGGAGAAGAUCUACCUGCCCCCCAUCCC |
| | | CGCCGACGAGAUCCGGAACCCCGUGGUGCUGCGGGCCCUGUCCCA |
| | | GGCCCGGAAGGUGAUCAACGGCGUGGUGCGGCGGUACGGCUCCCC |
| | | CGCCCGGAUCCACAUCGAGACCGCCCGGGAGGUGGGCAAGUCCUU |
| | | CAAGGACCGGAAGGAGAUCGAGAAGCGGCAGGAGGAGAACCGGA |
| | | AGGACCGGGAGAAGGCCGCCGCCAAGUUCCGGGAGUACUUCCCCA |
| | | ACUUCGUGGGCGAGCCCAAGUCCAAGGACAUCCUGAAGCUGCGGC |
| | | UGUACGAGCAGCAGCACGGCAAGUGCCUGUACUCCGGCAAGGAG |
| | | AUCAACCUGGUGCGGCUGAACGAGAAGGGCUACGUGGAGAUCGA |
| | | CCACGCCCUGCCCUUCUCCCGGACCUGGGACGACUCCUUCAACAA |
| | | CAAGGUGCUGGUGCUGGGCUCCGAGAACCAGAACAAGGGCAACC |
| | | AGACCCCCUACGAGUACUUCAACGGCAAGGACAACUCCCGGGAGU |
| | | GGCAGGAGUUCAAGGCCCGGGUGGAGACCUCCCGGUUCCCCCGGU |
| | | CCAAGAAGCAGCGGAUCCUGCUGCAGAAGUUCGACGAGGACGGC |
| | | UUCAAGGAGUGCAACCUGAACGACACCCGGUACGUGAACCGCUUC |
| | | CUGUGCCAGUUCGUGGCCGACCACAUCCUGCUGACCGGCAAGGGC |
| | | AAGCGGCGGGUGUUCGCCUCCAACGGCCAGAUCACCAACCUGCUG |
| | | CGGGGCUUCUGGGGCCUGCGGAAGGUGCGGGCCGAGAACGACCG |
| | | GCACCACGCCCUGGACGCCGUGGUGGUGGCCUGCUCCACCGUGGC |
| | | CAUGCAGCAGAAGAUCACCCGGUUCGUGCGGUACAAGGAGAUGA |
| | | ACGCCUUCGACGGCAAGACCAUCGACAAGGAGACCGGCAAGGUGC |
| | | UGCACCAGAAGACCCACUUCCCCCAGCCCUGGGAGUUCUUCGCCC |
| | | AGGAGGUGAUGAUCCGGGUGUUCGGCAAGCCCGACGGCAAGCCC |
| | | GAGUUCGAGGAGGCCGACACCCCCGAGAAGCUGCGGACCCUGCUG |
| | | GCCGAGAAGCUGUCCUCCCGGCCCGAGGCCGUGCACGAGUACGUG |
| | | ACCCCCCUGUUCGUGUCCCGGGCCCCCAACCGGAAGAUGUCCGGC |
| | | GCCCACAAGGACACCCUGCGGUCCGCCAAGCGGUUCGUGAAGCAC |
| | | AACGAGAAGAUCUCCGUGAAGCGGGUGUGGCUGACCGAGAUCAA |
| | | GCUGGCCGACCUGGAGAACAUGGUGAACUACAAGAACGGCCGGG |
| | | AGAUCGAGCUGUACGAGGCCCUGAAGGCCCGGCUGGAGGCCUACG |
| | | GCGGCAACGCCAAGCAGGCCUUCGACCCCAAGGACAACCCCUUCU |
| | | ACAAGAAGGGCGGCCAGCUGGUGAAGGCCGUGCGGGUGGAGAAG |
| | | ACCCAGGAGUCCGGCGUGCUGCUGAACAAGAAGAACGCCUACACC |
| | | AUCGCCGACAACGGCGACAUGGUGCGGGUGGACGUGUUCUGCAA |
| | | GGUGGACAAGAAGGGCAAGAACCAGUACUUCAUCGUGCCCAUCU |
| | | ACGCCUGGCAGGUGGCCGAGAACAUCCUGCCCGACAUCGACUGCA |
| | | AGGGCUACCGGAUCGACGACUCCUACACCUUCUGCUUCUCCCUGC |
| | | ACAAGUACGACCUGAUCGCCUUCCAGAAGGACGAGAAGUCCAAG |
| | | GUGGAGUUCGCCUACUACAUCAACUGCGACUCCUCCAACGGCCGG |
| | | UUCUACCUGGCCUGGCACGACAAGGGCUCCAAGGAGCAGCAGUUC |
| | | CGGAUCUCCACCCAGAACCUGGUGCUGAUCCAGAAGUACCAGGUG |
| | | AACGAGCUGGGCAAGGAGAUCCGGCCCUGCCGGCUGAAGAAGCG |
| | | GCCCCCCGUGCGGGAGGACAAGCGGCCCGCCGCCACCAAGAAGGC |
| | | CGGCCAGGCCAAGAAGAAGAAGUACCCCUACGACGUGCCCGACUA |
| | | CGCCGGCUACCCCUACGACGUGCCCGACUACGCCGGCUCCUACCC |
| | | CUACGACGUGCCCGACUACGCCGCCGCCCCCGCCGCCAAGAAGAA |
| | | GAAGCUGGACU |
| 142 | Exemplary amino | MDGSGGGSPKKKRKVEDKRPAATKKAGQAKKKKGGSGGGEASPASG |
| | acid sequence for | PRHLMDPHIFTSNFNNGIGRHKTYLCYEVERLDNGTSVKMDQHRGFLH |
| | APOBEC3A- | NQAKNLLCGFYGRHAELRFLDLVPSLQLDPAQIYRVTWFISWSPCFSW |
| | Nme2D16A | GCAGEVRAFLQENTHVRLRIFAARIYDYDPLYKEALQMLRDAGAQVSI |
| | | MTYDEFKHCWDTFVDHQGCPFQPWDGLDEHSQALSGRLRAILQNQG |
| | | NSGSETPGTSESATPESAAFKPNPINYILGLAIGIASVGWAMVEIDEEEN |
| | | PIRLIDLGVRVFERAEVPKTGDSLAMARRLARSVRRLTRRRAHRLLRA |
| | | RRLLKREGVLQAADFDENGLIKSLPNTPWQLRAAALDRKLTPLEWSA |
| | | VLLHLIKHRGYLSQRKNEGETADKELGALLKGVANNAHALQTGDFRT |
| | | PAELALNKFEKESGHIRNQRGDYSHTFSRKDLQAELILLFEKQKEFGNP |
| | | HVSGGLKEGIETLLMTQRPALSGDAVQKMLGHCTFEPAEPKAAKNTY |
| | | TAERFIWLTKLNNLRILEQGSERPLTDTERATLMDEPYRKSKLTYAQA |
| | | RKLLGLEDTAFFKGLRYGKDNAEASTLMEMKAYHAISRALEKEGLKD |
| | | KKSPLNLSSELQDEIGTAFSLFKTDEDITGRLKDRVQPEILEALLKHISFD |
| | | KFVQISLKALRRIVPLMEQGKRYDEACAEIYGDHYGKKNTEEKIYLPPI |
| | | PADEIRNPVVLRALSQARKVINGVVRRYGSPARIHIETAREVGKSFKDR |
| | | KEIEKRQEENRKDREKAAAKFREYFPNFVGEPKSKDILKLRLYEQQHG |
| | | KCLYSGKEINLVRLNEKGYVEIDHALPFSRTWDDSFNNKVLVLGSENQ |
| | | NKGNQTPYEYFNGKDNSREWQEFKARVETSRFPRSKKQRILLQKFDED |
| | | GFKECNLNDTRYVNRFLCQFVADHILLTGKGKRRVFASNGQITNLLRG |
| | | FWGLRKVRAENDRHHALDAVVVACSTVAMQQKITRFVRYKEMNAFD |
| | | GKTIDKETGKVLHQKTHFPQPWEFFAQEVMIRVFGKPDGKPEFEEADT |
| | | PEKLRTLLAEKLSSRPEAVHEYVTPLFVSRAPNRKMSGAHKDTLRSAK |
| | | RFVKHNEKISVKRVWLTEIKLADLENMVNYKNGREIELYEALKARLEA |
| | | YGGNAKQAFDPKDNPFYKKGGQLVKAVRVEKTQESGVLLNKKNAYT |
| | | IADNGDMVRVDVFCKVDKKGKNQYFIVPIYAWQVAENILPDIDCKGY |
| | | RIDDSYTFCFSLHKYDLIAFQKDEKSKVEFAYYINCDSSNGRFYLAWH |
| | | DKGSKEQQFRISTQNLVLIQKYQVNELGKEIRPCRLKKRPPVREDKRPA |
| | | ATKKAGQAKKKKYPYDVPDYAGYPYDVPDYAGSYPYDVPDYAAAPA |
| | | AKKKKLD |
| 143 | EXEMPLARY | GGGAAGCUCAGAAUAAACGCUCAACUUUGGCCGGAUCUGCCACCA |
| | MRNA | UGGACGGCUCCGGCGGCGGCUCCCCCAAGAAGAAGCGGAAGGUGG |
| | ENCODING | AGGACAAGCGGCCCGCCGCCACCAAGAAGGCCGGCCAGGCCAAGA |
| | APOBEC3A- | AGAAGAAGGGCGGCUCCGGCGGCGGCGAGGCCUCCCCCGCCUCCG |
| | NME2D16A | GCCCCCGGCACCUGAUGGACCCCCACAUCUUCACCUCCAACUUCA |
| | | ACAACGGCAUCGGCCGGCACAAGACCUACCUGUGCUACGAGGUGG |
| | | AGCGGCUGGACAACGGCACCUCCGUGAAGAUGGACCAGCACCGGG |
| | | GCUUCCUGCACAACCAGGCCAAGAACCUGCUGUGCGGCUUCUACG |
| | | GCCGGCACGCCGAGCUGCGGUUCCUGGACCUGGUGCCCUCCCUGC |
| | | AGCUGGACCCCGCCCAGAUCUACCGGGUGACCUGGUUCAUCUCCU |
| | | GGUCCCCCUGCUUCUCCUGGGGCUGCGCCGGCGAGGUGCGGGCCU |
| | | UCCUGCAGGAGAACACCCACGUGCGGCUGCGGAUCUUCGCCGCCC |
| | | GGAUCUACGACUACGACCCCCUGUACAAGGAGGCCCUGCAGAUGC |
| | | UGCGGGACGCCGGCGCCCAGGUGUCCAUCAUGACCUACGACGAGU |
| | | UCAAGCACUGCUGGGACACCUUCGUGGACCACCAGGGCUGCCCCU |
| | | UCCAGCCCUGGGACGGCCUGGACGAGCACUCCCAGGCCCUGUCCG |
| | | GCCGGCUGCGGGCCAUCCUGCAGAACCAGGGCAACUCCGGCUCCG |
| | | AGACCCCCGGCACCUCCGAGUCCGCCACCCCCGAGUCCGCAGCGU |
| | | UCAAACCAAAUCCCAUCAACUACAUCCUGGGCCUGGCCAUCGGCA |
| | | UCGCCUCCGUGGGCUGGGCCAUGGUGGAGAUCGACGAGGAGGAG |
| | | AACCCCAUCCGGCUGAUCGACCUGGGCGUGCGGGUGUUCGAGCGG |
| | | GCCGAGGUGCCCAAGACCGGCGACUCCCUGGCCAUGGCCCGGCGG |
| | | CUGGCCCGGUCCGUGCGGCGGCUGACCCGGCGGCGGGCCCACCGG |
| | | CUGCUGCGGGCCCGGCGGCUGCUGAAGCGGGAGGGCGUGCUGCAG |
| | | GCCGCCGACUUCGACGAGAACGGCCUGAUCAAGUCCCUGCCCAAC |
| | | ACCCCCUGGCAGCUGCGGGCCGCCGCCCUGGACCGGAAGCUGACC |
| | | CCCCUGGAGUGGUCCGCCGUGCUGCUGCACCUGAUCAAGCACCGG |
| | | GGCUACCUGUCCCAGCGGAAGAACGAGGGCGAGACCGCCGACAAG |
| | | GAGCUGGGCGCCCUGCUGAAGGGCGUGGCCAACAACGCCCACGCC |
| | | CUGCAGACCGGCGACUUCCGGACCCCCGCCGAGCUGGCCCUGAAC |
| | | AAGUUCGAGAAGGAGUCCGGCCACAUCCGGAACCAGCGGGGCGAC |
| | | UACUCCCACACCUUCUCCCGGAAGGACCUGCAGGCCGAGCUGAUC |
| | | CUGCUGUUCGAGAAGCAGAAGGAGUUCGGCAACCCCCACGUGUCC |
| | | GGCGGCCUGAAGGAGGGCAUCGAGACCCUGCUGAUGACCCAGCGG |
| | | CCCGCCCUGUCCGGCGACGCCGUGCAGAAGAUGCUGGGCCACUGC |
| | | ACCUUCGAGCCCGCCGAGCCCAAGGCCGCCAAGAACACCUACACC |
| | | GCCGAGCGGUUCAUCUGGCUGACCAAGCUGAACAACCUGCGGAUC |
| | | CUGGAGCAGGGCUCCGAGCGGCCCCUGACCGACACCGAGCGGGCC |
| | | ACCCUGAUGGACGAGCCCUACCGGAAGUCCAAGCUGACCUACGCC |
| | | CAGGCCCGGAAGCUGCUGGGCCUGGAGGACACCGCCUUCUUCAAG |
| | | GGCCUGCGGUACGGCAAGGACAACGCCGAGGCCUCCACCCUGAUG |
| | | GAGAUGAAGGCCUACCACGCCAUCUCCCGGGCCCUGGAGAAGGAG |
| | | GGCCUGAAGGACAAGAAGUCCCCCCUGAACCUGUCCUCCGAGCUG |
| | | CAGGACGAGAUCGGCACCGCCUUCUCCCUGUUCAAGACCGACGAG |
| | | GACAUCACCGGCCGGCUGAAGGACCGGGUGCAGCCCGAGAUCCUG |
| | | GAGGCCCUGCUGAAGCACAUCUCCUUCGACAAGUUCGUGCAGAUC |
| | | UCCCUGAAGGCCCUGCGGCGGAUCGUGCCCCUGAUGGAGCAGGGC |
| | | AAGCGGUACGACGAGGCCUGCGCCGAGAUCUACGGCGACCACUAC |
| | | GGCAAGAAGAACACCGAGGAGAAGAUCUACCUGCCCCCCAUCCCC |
| | | GCCGACGAGAUCCGGAACCCCGUGGUGCUGCGGGCCCUGUCCCAG |
| | | GCCCGGAAGGUGAUCAACGGCGUGGUGCGGCGGUACGGCUCCCCC |
| | | GCCCGGAUCCACAUCGAGACCGCCCGGGAGGUGGGCAAGUCCUUC |
| | | AAGGACCGGAAGGAGAUCGAGAAGCGGCAGGAGGAGAACCGGAA |
| | | GGACCGGGAGAAGGCCGCCGCCAAGUUCCGGGAGUACUUCCCCAA |
| | | CUUCGUGGGCGAGCCCAAGUCCAAGGACAUCCUGAAGCUGCGGCU |
| | | GUACGAGCAGCAGCACGGCAAGUGCCUGUACUCCGGCAAGGAGA |
| | | UCAACCUGGUGCGGCUGAACGAGAAGGGCUACGUGGAGAUCGAC |
| | | CACGCCCUGCCCUUCUCCCGGACCUGGGACGACUCCUUCAACAAC |
| | | AAGGUGCUGGUGCUGGGCUCCGAGAACCAGAACAAGGGCAACCA |
| | | GACCCCCUACGAGUACUUCAACGGCAAGGACAACUCCCGGGAGUG |
| | | GCAGGAGUUCAAGGCCCGGGUGGAGACCUCCCGGUUCCCCCGGUC |
| | | CAAGAAGCAGCGGAUCCUGCUGCAGAAGUUCGACGAGGACGGCU |
| | | UCAAGGAGUGCAACCUGAACGACACCCGGUACGUGAACCGCUUCC |
| | | UGUGCCAGUUCGUGGCCGACCACAUCCUGCUGACCGGCAAGGGCA |
| | | AGCGGCGGGUGUUCGCCUCCAACGGCCAGAUCACCAACCUGCUGC |
| | | GGGGCUUCUGGGGCCUGCGGAAGGUGCGGGCCGAGAACGACCGG |
| | | CACCACGCCCUGGACGCCGUGGUGGUGGCCUGCUCCACCGUGGCC |
| | | AUGCAGCAGAAGAUCACCCGGUUCGUGCGGUACAAGGAGAUGAA |
| | | CGCCUUCGACGGCAAGACCAUCGACAAGGAGACCGGCAAGGUGCU |
| | | GCACCAGAAGACCCACUUCCCCCAGCCCUGGGAGUUCUUCGCCCA |
| | | GGAGGUGAUGAUCCGGGUGUUCGGCAAGCCCGACGGCAAGCCCG |
| | | AGUUCGAGGAGGCCGACACCCCCGAGAAGCUGCGGACCCUGCUGG |
| | | CCGAGAAGCUGUCCUCCCGGCCCGAGGCCGUGCACGAGUACGUGA |
| | | CCCCCCUGUUCGUGUCCCGGGCCCCCAACCGGAAGAUGUCCGGCG |
| | | CCCACAAGGACACCCUGCGGUCCGCCAAGCGGUUCGUGAAGCACA |
| | | ACGAGAAGAUCUCCGUGAAGCGGGUGUGGCUGACCGAGAUCAAG |
| | | CUGGCCGACCUGGAGAACAUGGUGAACUACAAGAACGGCCGGGA |
| | | GAUCGAGCUGUACGAGGCCCUGAAGGCCCGGCUGGAGGCCUACGG |
| | | CGGCAACGCCAAGCAGGCCUUCGACCCCAAGGACAACCCCUUCUA |
| | | CAAGAAGGGCGGCCAGCUGGUGAAGGCCGUGCGGGUGGAGAAGA |
| | | CCCAGGAGUCCGGCGUGCUGCUGAACAAGAAGAACGCCUACACCA |
| | | UCGCCGACAACGGCGACAUGGUGCGGGUGGACGUGUUCUGCAAG |
| | | GUGGACAAGAAGGGCAAGAACCAGUACUUCAUCGUGCCCAUCUA |
| | | CGCCUGGCAGGUGGCCGAGAACAUCCUGCCCGACAUCGACUGCAA |
| | | GGGCUACCGGAUCGACGACUCCUACACCUUCUGCUUCUCCCUGCA |
| | | CAAGUACGACCUGAUCGCCUUCCAGAAGGACGAGAAGUCCAAGG |
| | | UGGAGUUCGCCUACUACAUCAACUGCGACUCCUCCAACGGCCGGU |
| | | UCUACCUGGCCUGGCACGACAAGGGCUCCAAGGAGCAGCAGUUCC |
| | | GGAUCUCCACCCAGAACCUGGUGCUGAUCCAGAAGUACCAGGUGA |
| | | ACGAGCUGGGCAAGGAGAUCCGGCCCUGCCGGCUGAAGAAGCGGC |
| | | CCCCCGUGCGGUAGUGACUAGCACCAGCCUCAAGAACACCCGAAU |
| | | GGAGUCUCUAAGCUACAUAAUACCAACUUACACUUUACAAAAUG |
| | | UUGUCCCCCAAAAUGUAGCCAUUCGUAUCUGCUCCUAAUAAAAA |
| | | GAAAGUUUCUUCACAUUCU |
| 144 | Exemplary open | AUGGACGGCUCCGGCGGCGGCUCCCCCAAGAAGAAGCGGAAGGUG |
| | reading frame for | GAGGACAAGCGGCCCGCCGCCACCAAGAAGGCCGGCCAGGCCAAG |
| | APOBEC3A- | AAGAAGAAGGGCGGCUCCGGCGGCGGCGAGGCCUCCCCCGCCUCC |
| | Nme2D16A | GGCCCCCGGCACCUGAUGGACCCCCACAUCUUCACCUCCAACUUC |
| | | AACAACGGCAUCGGCCGGCACAAGACCUACCUGUGCUACGAGGUG |
| | | GAGCGGCUGGACAACGGCACCUCCGUGAAGAUGGACCAGCACCGG |
| | | GGCUUCCUGCACAACCAGGCCAAGAACCUGCUGUGCGGCUUCUAC |
| | | GGCCGGCACGCCGAGCUGCGGUUCCUGGACCUGGUGCCCUCCCUG |
| | | CAGCUGGACCCCGCCCAGAUCUACCGGGUGACCUGGUUCAUCUCC |
| | | UGGUCCCCCUGCUUCUCCUGGGGCUGCGCCGGCGAGGUGCGGGCC |
| | | UUCCUGCAGGAGAACACCCACGUGCGGCUGCGGAUCUUCGCCGCC |
| | | CGGAUCUACGACUACGACCCCCUGUACAAGGAGGCCCUGCAGAUG |
| | | CUGCGGGACGCCGGCGCCCAGGUGUCCAUCAUGACCUACGACGAG |
| | | UUCAAGCACUGCUGGGACACCUUCGUGGACCACCAGGGCUGCCCC |
| | | UUCCAGCCCUGGGACGGCCUGGACGAGCACUCCCAGGCCCUGUCC |
| | | GGCCGGCUGCGGGCCAUCCUGCAGAACCAGGGCAACUCCGGCUCC |
| | | GAGACCCCCGGCACCUCCGAGUCCGCCACCCCCGAGUCCGCAGCG |
| | | UUCAAACCAAAUCCCAUCAACUACAUCCUGGGCCUGGCCAUCGGC |
| | | AUCGCCUCCGUGGGCUGGGCCAUGGUGGAGAUCGACGAGGAGGA |
| | | GAACCCCAUCCGGCUGAUCGACCUGGGCGUGCGGGUGUUCGAGCG |
| | | GGCCGAGGUGCCCAAGACCGGCGACUCCCUGGCCAUGGCCCGGCG |
| | | GCUGGCCCGGUCCGUGCGGCGGCUGACCCGGCGGCGGGCCCACCG |
| | | GCUGCUGCGGGCCCGGCGGCUGCUGAAGCGGGAGGGCGUGCUGCA |
| | | GGCCGCCGACUUCGACGAGAACGGCCUGAUCAAGUCCCUGCCCAA |
| | | CACCCCCUGGCAGCUGCGGGCCGCCGCCCUGGACCGGAAGCUGAC |
| | | CCCCCUGGAGUGGUCCGCCGUGCUGCUGCACCUGAUCAAGCACCG |
| | | GGGCUACCUGUCCCAGCGGAAGAACGAGGGCGAGACCGCCGACAA |
| | | GGAGCUGGGCGCCCUGCUGAAGGGCGUGGCCAACAACGCCCACGC |
| | | CCUGCAGACCGGCGACUUCCGGACCCCCGCCGAGCUGGCCCUGAA |
| | | CAAGUUCGAGAAGGAGUCCGGCCACAUCCGGAACCAGCGGGGCGA |
| | | CUACUCCCACACCUUCUCCCGGAAGGACCUGCAGGCCGAGCUGAU |
| | | CCUGCUGUUCGAGAAGCAGAAGGAGUUCGGCAACCCCCACGUGUC |
| | | CGGCGGCCUGAAGGAGGGCAUCGAGACCCUGCUGAUGACCCAGCG |
| | | GCCCGCCCUGUCCGGCGACGCCGUGCAGAAGAUGCUGGGCCACUG |
| | | CACCUUCGAGCCCGCCGAGCCCAAGGCCGCCAAGAACACCUACAC |
| | | CGCCGAGCGGUUCAUCUGGCUGACCAAGCUGAACAACCUGCGGAU |
| | | CCUGGAGCAGGGCUCCGAGCGGCCCCUGACCGACACCGAGCGGGC |
| | | CACCCUGAUGGACGAGCCCUACCGGAAGUCCAAGCUGACCUACGC |
| | | CCAGGCCCGGAAGCUGCUGGGCCUGGAGGACACCGCCUUCUUCAA |
| | | GGGCCUGCGGUACGGCAAGGACAACGCCGAGGCCUCCACCCUGAU |
| | | GGAGAUGAAGGCCUACCACGCCAUCUCCCGGGCCCUGGAGAAGGA |
| | | GGGCCUGAAGGACAAGAAGUCCCCCCUGAACCUGUCCUCCGAGCU |
| | | GCAGGACGAGAUCGGCACCGCCUUCUCCCUGUUCAAGACCGACGA |
| | | GGACAUCACCGGCCGGCUGAAGGACCGGGUGCAGCCCGAGAUCCU |
| | | GGAGGCCCUGCUGAAGCACAUCUCCUUCGACAAGUUCGUGCAGAU |
| | | CUCCCUGAAGGCCCUGCGGCGGAUCGUGCCCCUGAUGGAGCAGGG |
| | | CAAGCGGUACGACGAGGCCUGCGCCGAGAUCUACGGCGACCACUA |
| | | CGGCAAGAAGAACACCGAGGAGAAGAUCUACCUGCCCCCCAUCCC |
| | | CGCCGACGAGAUCCGGAACCCCGUGGUGCUGCGGGCCCUGUCCCA |
| | | GGCCCGGAAGGUGAUCAACGGCGUGGUGCGGCGGUACGGCUCCCC |
| | | CGCCCGGAUCCACAUCGAGACCGCCCGGGAGGUGGGCAAGUCCUU |
| | | CAAGGACCGGAAGGAGAUCGAGAAGCGGCAGGAGGAGAACCGGA |
| | | AGGACCGGGAGAAGGCCGCCGCCAAGUUCCGGGAGUACUUCCCCA |
| | | ACUUCGUGGGCGAGCCCAAGUCCAAGGACAUCCUGAAGCUGCGGC |
| | | UGUACGAGCAGCAGCACGGCAAGUGCCUGUACUCCGGCAAGGAG |
| | | AUCAACCUGGUGCGGCUGAACGAGAAGGGCUACGUGGAGAUCGA |
| | | CCACGCCCUGCCCUUCUCCCGGACCUGGGACGACUCCUUCAACAA |
| | | CAAGGUGCUGGUGCUGGGCUCCGAGAACCAGAACAAGGGCAACC |
| | | AGACCCCCUACGAGUACUUCAACGGCAAGGACAACUCCCGGGAGU |
| | | GGCAGGAGUUCAAGGCCCGGGUGGAGACCUCCCGGUUCCCCCGGU |
| | | CCAAGAAGCAGCGGAUCCUGCUGCAGAAGUUCGACGAGGACGGC |
| | | UUCAAGGAGUGCAACCUGAACGACACCCGGUACGUGAACCGCUUC |
| | | CUGUGCCAGUUCGUGGCCGACCACAUCCUGCUGACCGGCAAGGGC |
| | | AAGCGGCGGGUGUUCGCCUCCAACGGCCAGAUCACCAACCUGCUG |
| | | CGGGGCUUCUGGGGCCUGCGGAAGGUGCGGGCCGAGAACGACCG |
| | | GCACCACGCCCUGGACGCCGUGGUGGUGGCCUGCUCCACCGUGGC |
| | | CAUGCAGCAGAAGAUCACCCGGUUCGUGCGGUACAAGGAGAUGA |
| | | ACGCCUUCGACGGCAAGACCAUCGACAAGGAGACCGGCAAGGUGC |
| | | UGCACCAGAAGACCCACUUCCCCCAGCCCUGGGAGUUCUUCGCCC |
| | | AGGAGGUGAUGAUCCGGGUGUUCGGCAAGCCCGACGGCAAGCCC |
| | | GAGUUCGAGGAGGCCGACACCCCCGAGAAGCUGCGGACCCUGCUG |
| | | GCCGAGAAGCUGUCCUCCCGGCCCGAGGCCGUGCACGAGUACGUG |
| | | ACCCCCCUGUUCGUGUCCCGGGCCCCCAACCGGAAGAUGUCCGGC |
| | | GCCCACAAGGACACCCUGCGGUCCGCCAAGCGGUUCGUGAAGCAC |
| | | AACGAGAAGAUCUCCGUGAAGCGGGUGUGGCUGACCGAGAUCAA |
| | | GCUGGCCGACCUGGAGAACAUGGUGAACUACAAGAACGGCCGGG |
| | | AGAUCGAGCUGUACGAGGCCCUGAAGGCCCGGCUGGAGGCCUACG |
| | | GCGGCAACGCCAAGCAGGCCUUCGACCCCAAGGACAACCCCUUCU |
| | | ACAAGAAGGGCGGCCAGCUGGUGAAGGCCGUGCGGGUGGAGAAG |
| | | ACCCAGGAGUCCGGCGUGCUGCUGAACAAGAAGAACGCCUACACC |
| | | AUCGCCGACAACGGCGACAUGGUGCGGGUGGACGUGUUCUGCAA |
| | | GGUGGACAAGAAGGGCAAGAACCAGUACUUCAUCGUGCCCAUCU |
| | | ACGCCUGGCAGGUGGCCGAGAACAUCCUGCCCGACAUCGACUGCA |
| | | AGGGCUACCGGAUCGACGACUCCUACACCUUCUGCUUCUCCCUGC |
| | | ACAAGUACGACCUGAUCGCCUUCCAGAAGGACGAGAAGUCCAAG |
| | | GUGGAGUUCGCCUACUACAUCAACUGCGACUCCUCCAACGGCCGG |
| | | UUCUACCUGGCCUGGCACGACAAGGGCUCCAAGGAGCAGCAGUUC |
| | | CGGAUCUCCACCCAGAACCUGGUGCUGAUCCAGAAGUACCAGGUG |
| | | AACGAGCUGGGCAAGGAGAUCCGGCCCUGCCGGCUGAAGAAGCG |
| | | GCCCCCCGUGCGGUAG |
| 145 | Exemplary amino | MDGSGGGSPKKKRKVEDKRPAATKKAGQAKKKKGGSGGGEASPASG |
| | acid sequence for | PRHLMDPHIFTSNFNNGIGRHKTYLCYEVERLDNGTSVKMDQHRGFLH |
| | APOBEC3A- | NQAKNLLCGFYGRHAELRFLDLVPSLQLDPAQIYRVTWFISWSPCFSW |
| | Nme2D16A | GCAGEVRAFLQENTHVRLRIFAARIYDYDPLYKEALQMLRDAGAQVSI |
| | | MTYDEFKHCWDTFVDHQGCPFQPWDGLDEHSQALSGRLRAILQNQG |
| | | NSGSETPGTSESATPESAAFKPNPINYILGLAIGIASVGWAMVEIDEEEN |
| | | PIRLIDLGVRVFERAEVPKTGDSLAMARRLARSVRRLTRRRAHRLLRA |
| | | RRLLKREGVLQAADFDENGLIKSLPNTPWQLRAAALDRKLTPLEWSA |
| | | VLLHLIKHRGYLSQRKNEGETADKELGALLKGVANNAHALQTGDFRT |
| | | PAELALNKFEKESGHIRNQRGDYSHTFSRKDLQAELILLFEKQKEFGNP |
| | | HVSGGLKEGIETLLMTQRPALSGDAVQKMLGHCTFEPAEPKAAKNTY |
| | | TAERFIWLTKLNNLRILEQGSERPLTDTERATLMDEPYRKSKLTYAQA |
| | | RKLLGLEDTAFFKGLRYGKDNAEASTLMEMKAYHAISRALEKEGLKD |
| | | KKSPLNLSSELQDEIGTAFSLFKTDEDITGRLKDRVQPEILEALLKHISFD |
| | | KFVQISLKALRRIVPLMEQGKRYDEACAEIYGDHYGKKNTEEKIYLPPI |
| | | PADEIRNPVVLRALSQARKVINGVVRRYGSPARIHIETAREVGKSFKDR |
| | | KEIEKRQEENRKDREKAAAKFREYFPNFVGEPKSKDILKLRLYEQQHG |
| | | KCLYSGKEINLVRLNEKGYVEIDHALPFSRTWDDSFNNKVLVLGSENQ |
| | | NKGNQTPYEYFNGKDNSREWQEFKARVETSRFPRSKKQRILLQKFDED |
| | | GFKECNLNDTRYVNRFLCQFVADHILLTGKGKRRVFASNGQITNLLRG |
| | | FWGLRKVRAENDRHHALDAVVVACSTVAMQQKITRFVRYKEMNAFD |
| | | GKTIDKETGKVLHQKTHFPQPWEFFAQEVMIRVFGKPDGKPEFEEADT |
| | | PEKLRTLLAEKLSSRPEAVHEYVTPLFVSRAPNRKMSGAHKDTLRSAK |
| | | RFVKHNEKISVKRVWLTEIKLADLENMVNYKNGREIELYEALKARLEA |
| | | YGGNAKQAFDPKDNPFYKKGGQLVKAVRVEKTQESGVLLNKKNAYT |
| | | IADNGDMVRVDVFCKVDKKGKNQYFIVPIYAWQVAENILPDIDCKGY |
| | | RIDDSYTFCFSLHKYDLIAFQKDEKSKVEFAYYINCDSSNGRFYLAWH |
| | | DKGSKEQQFRISTQNLVLIQKYQVNELGKEIRPCRLKKRPPVR* |
| 146 | Exemplary amino | MDGSGGGSPKKKRKVEDKRPAATKKAGQAKKKKGGSGGGEASPASG |
| | acid sequence for | PRHLMDPHIFTSNFNNGIGRHKTYLCYEVERLDNGTSVKMDQHRGFLH |
| | NLS-NLS- | NQAKNLLCGFYGRHAELRFLDLVPSLQLDPAQIYRVTWFISWSPCFSW |
| | APOBEC3A- | GCAGEVRAFLQENTHVRLRIFAARIYDYDPLYKEALQMLRDAGAQVSI |
| | L070-Nme2D16A | MTYDEFKHCWDTFVDHQGCPFQPWDGLDEHSQALSGRLRAILQNQG |
| | | NGTKDSTKDIPETPSKDAAFKPNPINYILGLAIGIASVGWAMVEIDEEEN |
| | | PIRLIDLGVRVFERAEVPKTGDSLAMARRLARSVRRLTRRRAHRLLRA |
| | | RRLLKREGVLQAADFDENGLIKSLPNTPWQLRAAALDRKLTPLEWSA |
| | | VLLHLIKHRGYLSQRKNEGETADKELGALLKGVANNAHALQTGDFRT |
| | | PAELALNKFEKESGHIRNQRGDYSHTFSRKDLQAELILLFEKQKEFGNP |
| | | HVSGGLKEGIETLLMTQRPALSGDAVQKMLGHCTFEPAEPKAAKNTY |
| | | TAERFIWLTKLNNLRILEQGSERPLTDTERATLMDEPYRKSKLTYAQA |
| | | RKLLGLEDTAFFKGLRYGKDNAEASTLMEMKAYHAISRALEKEGLKD |
| | | KKSPLNLSSELQDEIGTAFSLFKTDEDITGRLKDRVQPEILEALLKHISFD |
| | | KFVQISLKALRRIVPLMEQGKRYDEACAEIYGDHYGKKNTEEKIYLPPI |
| | | PADEIRNPVVLRALSQARKVINGVVRRYGSPARIHIETAREVGKSFKDR |
| | | KEIEKRQEENRKDREKAAAKFREYFPNFVGEPKSKDILKLRLYEQQHG |
| | | KCLYSGKEINLVRLNEKGYVEIDHALPFSRTWDDSFNNKVLVLGSENQ |
| | | NKGNQTPYEYFNGKDNSREWQEFKARVETSRFPRSKKQRILLQKFDED |
| | | GFKECNLNDTRYVNRFLCQFVADHILLTGKGKRRVFASNGQITNLLRG |
| | | FWGLRKVRAENDRHHALDAVVVACSTVAMQQKITRFVRYKEMNAFD |
| | | GKTIDKETGKVLHQKTHFPQPWEFFAQEVMIRVFGKPDGKPEFEEADT |
| | | PEKLRTLLAEKLSSRPEAVHEYVTPLFVSRAPNRKMSGAHKDTLRSAK |
| | | RFVKHNEKISVKRVWLTEIKLADLENMVNYKNGREIELYEALKARLEA |
| | | YGGNAKQAFDPKDNPFYKKGGQLVKAVRVEKTQESGVLLNKKNAYT |
| | | IADNGDMVRVDVFCKVDKKGKNQYFIVPIYAWQVAENILPDIDCKGY |
| | | RIDDSYTFCFSLHKYDLIAFQKDEKSKVEFAYYINCDSSNGRFYLAWH |
| | | DKGSKEQQFRISTQNLVLIQKYQVNELGKEIRPCRLKKRPPVR |
| 147 | mRNA encoding | GGGAAGCUCAGAAUAAACGCUCAACUUUGGCCGGAUCUGCCACCA |
| | BC22- | UGGACGGCUCCGGCGGCGGCUCCCCCAAGAAGAAGCGGAAGGUGG |
| | Nme2D16A | AGGACAAGCGGCCCGCCGCCACCAAGAAGGCCGGCCAGGCCAAGA |
| | (Nme2 BC22n) | AGAAGAAGGGCGGCUCCGGCGGCGGCGAGGCCUCCCCCGCCUCCG |
| | | GCCCCCGGCACCUGAUGGACCCCCACAUCUUCACCUCCAACUUCA |
| | | ACAACGGCAUCGGCCGGCACAAGACCUACCUGUGCUACGAGGUGG |
| | | AGCGGCUGGACAACGGCACCUCCGUGAAGAUGGACCAGCACCGGG |
| | | GCUUCCUGCACAACCAGGCCAAGAACCUGCUGUGCGGCUUCUACG |
| | | GCCGGCACGCCGAGCUGCGGUUCCUGGACCUGGUGCCCUCCCUGC |
| | | AGCUGGACCCCGCCCAGAUCUACCGGGUGACCUGGUUCAUCUCCU |
| | | GGUCCCCCUGCUUCUCCUGGGGCUGCGCCGGCGAGGUGCGGGCCU |
| | | UCCUGCAGGAGAACACCCACGUGCGGCUGCGGAUCUUCGCCGCCC |
| | | GGAUCUACGACUACGACCCCCUGUACAAGGAGGCCCUGCAGAUGC |
| | | UGCGGGACGCCGGCGCCCAGGUGUCCAUCAUGACCUACGACGAGU |
| | | UCAAGCACUGCUGGGACACCUUCGUGGACCACCAGGGCUGCCCCU |
| | | UCCAGCCCUGGGACGGCCUGGACGAGCACUCCCAGGCCCUGUCCG |
| | | GCCGGCUGCGGGCCAUCCUGCAGAACCAGGGCAACUCCGGCUCCG |
| | | AGACCCCCGGCACCUCCGAGUCCGCCACCCCCGAGUCCGCAGCGU |
| | | UCAAACCAAAUCCCAUCAACUACAUCCUGGGCCUGGCCAUCGGCA |
| | | UCGCCUCCGUGGGCUGGGCCAUGGUGGAGAUCGACGAGGAGGAG |
| | | AACCCCAUCCGGCUGAUCGACCUGGGCGUGCGGGUGUUCGAGCGG |
| | | GCCGAGGUGCCCAAGACCGGCGACUCCCUGGCCAUGGCCCGGCGG |
| | | CUGGCCCGGUCCGUGCGGCGGCUGACCCGGCGGCGGGCCCACCGG |
| | | CUGCUGCGGGCCCGGCGGCUGCUGAAGCGGGAGGGCGUGCUGCAG |
| | | GCCGCCGACUUCGACGAGAACGGCCUGAUCAAGUCCCUGCCCAAC |
| | | ACCCCCUGGCAGCUGCGGGCCGCCGCCCUGGACCGGAAGCUGACC |
| | | CCCCUGGAGUGGUCCGCCGUGCUGCUGCACCUGAUCAAGCACCGG |
| | | GGCUACCUGUCCCAGCGGAAGAACGAGGGCGAGACCGCCGACAAG |
| | | GAGCUGGGCGCCCUGCUGAAGGGCGUGGCCAACAACGCCCACGCC |
| | | CUGCAGACCGGCGACUUCCGGACCCCCGCCGAGCUGGCCCUGAAC |
| | | AAGUUCGAGAAGGAGUCCGGCCACAUCCGGAACCAGCGGGGCGAC |
| | | UACUCCCACACCUUCUCCCGGAAGGACCUGCAGGCCGAGCUGAUC |
| | | CUGCUGUUCGAGAAGCAGAAGGAGUUCGGCAACCCCCACGUGUCC |
| | | GGCGGCCUGAAGGAGGGCAUCGAGACCCUGCUGAUGACCCAGCGG |
| | | CCCGCCCUGUCCGGCGACGCCGUGCAGAAGAUGCUGGGCCACUGC |
| | | ACCUUCGAGCCCGCCGAGCCCAAGGCCGCCAAGAACACCUACACC |
| | | GCCGAGCGGUUCAUCUGGCUGACCAAGCUGAACAACCUGCGGAUC |
| | | CUGGAGCAGGGCUCCGAGCGGCCCCUGACCGACACCGAGCGGGCC |
| | | ACCCUGAUGGACGAGCCCUACCGGAAGUCCAAGCUGACCUACGCC |
| | | CAGGCCCGGAAGCUGCUGGGCCUGGAGGACACCGCCUUCUUCAAG |
| | | GGCCUGCGGUACGGCAAGGACAACGCCGAGGCCUCCACCCUGAUG |
| | | GAGAUGAAGGCCUACCACGCCAUCUCCCGGGCCCUGGAGAAGGAG |
| | | GGCCUGAAGGACAAGAAGUCCCCCCUGAACCUGUCCUCCGAGCUG |
| | | CAGGACGAGAUCGGCACCGCCUUCUCCCUGUUCAAGACCGACGAG |
| | | GACAUCACCGGCCGGCUGAAGGACCGGGUGCAGCCCGAGAUCCUG |
| | | GAGGCCCUGCUGAAGCACAUCUCCUUCGACAAGUUCGUGCAGAUC |
| | | UCCCUGAAGGCCCUGCGGCGGAUCGUGCCCCUGAUGGAGCAGGGC |
| | | AAGCGGUACGACGAGGCCUGCGCCGAGAUCUACGGCGACCACUAC |
| | | GGCAAGAAGAACACCGAGGAGAAGAUCUACCUGCCCCCCAUCCCC |
| | | GCCGACGAGAUCCGGAACCCCGUGGUGCUGCGGGCCCUGUCCCAG |
| | | GCCCGGAAGGUGAUCAACGGCGUGGUGCGGCGGUACGGCUCCCCC |
| | | GCCCGGAUCCACAUCGAGACCGCCCGGGAGGUGGGCAAGUCCUUC |
| | | AAGGACCGGAAGGAGAUCGAGAAGCGGCAGGAGGAGAACCGGAA |
| | | GGACCGGGAGAAGGCCGCCGCCAAGUUCCGGGAGUACUUCCCCAA |
| | | CUUCGUGGGCGAGCCCAAGUCCAAGGACAUCCUGAAGCUGCGGCU |
| | | GUACGAGCAGCAGCACGGCAAGUGCCUGUACUCCGGCAAGGAGA |
| | | UCAACCUGGUGCGGCUGAACGAGAAGGGCUACGUGGAGAUCGAC |
| | | CACGCCCUGCCCUUCUCCCGGACCUGGGACGACUCCUUCAACAAC |
| | | AAGGUGCUGGUGCUGGGCUCCGAGAACCAGAACAAGGGCAACCA |
| | | GACCCCCUACGAGUACUUCAACGGCAAGGACAACUCCCGGGAGUG |
| | | GCAGGAGUUCAAGGCCCGGGUGGAGACCUCCCGGUUCCCCCGGUC |
| | | CAAGAAGCAGCGGAUCCUGCUGCAGAAGUUCGACGAGGACGGCU |
| | | UCAAGGAGUGCAACCUGAACGACACCCGGUACGUGAACCGCUUCC |
| | | UGUGCCAGUUCGUGGCCGACCACAUCCUGCUGACCGGCAAGGGCA |
| | | AGCGGCGGGUGUUCGCCUCCAACGGCCAGAUCACCAACCUGCUGC |
| | | GGGGCUUCUGGGGCCUGCGGAAGGUGCGGGCCGAGAACGACCGG |
| | | CACCACGCCCUGGACGCCGUGGUGGUGGCCUGCUCCACCGUGGCC |
| | | AUGCAGCAGAAGAUCACCCGGUUCGUGCGGUACAAGGAGAUGAA |
| | | CGCCUUCGACGGCAAGACCAUCGACAAGGAGACCGGCAAGGUGCU |
| | | GCACCAGAAGACCCACUUCCCCCAGCCCUGGGAGUUCUUCGCCCA |
| | | GGAGGUGAUGAUCCGGGUGUUCGGCAAGCCCGACGGCAAGCCCG |
| | | AGUUCGAGGAGGCCGACACCCCCGAGAAGCUGCGGACCCUGCUGG |
| | | CCGAGAAGCUGUCCUCCCGGCCCGAGGCCGUGCACGAGUACGUGA |
| | | CCCCCCUGUUCGUGUCCCGGGCCCCCAACCGGAAGAUGUCCGGCG |
| | | CCCACAAGGACACCCUGCGGUCCGCCAAGCGGUUCGUGAAGCACA |
| | | ACGAGAAGAUCUCCGUGAAGCGGGUGUGGCUGACCGAGAUCAAG |
| | | CUGGCCGACCUGGAGAACAUGGUGAACUACAAGAACGGCCGGGA |
| | | GAUCGAGCUGUACGAGGCCCUGAAGGCCCGGCUGGAGGCCUACGG |
| | | CGGCAACGCCAAGCAGGCCUUCGACCCCAAGGACAACCCCUUCUA |
| | | CAAGAAGGGCGGCCAGCUGGUGAAGGCCGUGCGGGUGGAGAAGA |
| | | CCCAGGAGUCCGGCGUGCUGCUGAACAAGAAGAACGCCUACACCA |
| | | UCGCCGACAACGGCGACAUGGUGCGGGUGGACGUGUUCUGCAAG |
| | | GUGGACAAGAAGGGCAAGAACCAGUACUUCAUCGUGCCCAUCUA |
| | | CGCCUGGCAGGUGGCCGAGAACAUCCUGCCCGACAUCGACUGCAA |
| | | GGGCUACCGGAUCGACGACUCCUACACCUUCUGCUUCUCCCUGCA |
| | | CAAGUACGACCUGAUCGCCUUCCAGAAGGACGAGAAGUCCAAGG |
| | | UGGAGUUCGCCUACUACAUCAACUGCGACUCCUCCAACGGCCGGU |
| | | UCUACCUGGCCUGGCACGACAAGGGCUCCAAGGAGCAGCAGUUCC |
| | | GGAUCUCCACCCAGAACCUGGUGCUGAUCCAGAAGUACCAGGUGA |
| | | ACGAGCUGGGCAAGGAGAUCCGGCCCUGCCGGCUGAAGAAGCGGC |
| | | CCCCCGUGCGGUAGUGACUAGCACCAGCCUCAAGAACACCCGAAU |
| | | GGAGUCUCUAAGCUACAUAAUACCAACUUACACUUUACAAAAUG |
| | | UUGUCCCCCAAAAUGUAGCCAUUCGUAUCUGCUCCUAAUAAAAA |
| | | GAAAGUUUCUUCACAUUCUCUCGAGAAAAAAAAAAAAUGGAAAA |
| | | AAAAAAAACGGAAAAAAAAAAAGGUAAAAAAAAAAAAUAUAAAA |
| | | AAAAAAACAUAAAAAAAAAAAACGAAAAAAAAAAAACGUAAAAA |
| | | AAAAAAACUCAAAAAAAAAAAGAUAAAAAAAAAAAACCUAAAAA |
| | | AAAAAAAUGUAAAAAAAAAAAAGGGAAAAAAAAAAACGCAAAAA |
| | | AAAAAAACACAAAAAAAAAAAAUGCAAAAAAAAAAAAUCGAAAA |
| | | AAAAAAAAUCUAAAAAAAAAAAACGAAAAAAAAAAAACCCAAAA |
| | | AAAAAAAAGACAAAAAAAAAAAAUAGAAAAAAAAAAAGUUAAAA |
| | | AAAAAAAACUGAAAAAAAAAAAAUUUAAAAAAAAAAAAUCUAG |
| 148 | Amino acid | MEASPASGPRHLMDPHIFTSNFNNGIGRHKTYLCYEVERLDNGTSVKM |
| | sequence for base | DQHRGFLHNQAKNLLCGFYGRHAELRFLDLVPSLQLDPAQIYRVTWFI |
| | editor with linker | SWSPCFSWGCAGEVRAFLQENTHVRLRIFAARIYDYDPLYKEALQMLR |
| | L070 | DAGAQVSIMTYDEFKHCWDTFVDHQGCPFQPWDGLDEHSQALSGRL |
| | | RAILQNQGNGTKDSTKDIPETPSKDDKKYSIGLAIGTNSVGWAVITDEY |
| | | KVPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGNIV |
| | | DEVAYHEKYPTIYHLRKKLVDSTDKADLRLIYLALAHMIKFRGHFLIE |
| | | GDLNPDNSDVDKLFIQLVQTYNQLFEENPINASGVDAKAILSARLSKSR |
| | | RLENLIAQLPGEKKNGLFGNLIALSLGLTPNFKSNFDLAEDAKLQLSKD |
| | | TYDDDLDNLLAQIGDQYADLFLAAKNLSDAILLSDILRVNTEITKAPLS |
| | | ASMIKRYDEHHQDLTLLKALVRQQLPEKYKEIFFDQSKNGYAGYIDGG |
| | | ASQEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAWM |
| | | TRKSEETITPWNFEEVVDKGASAQSFIERMTNFDKNLPNEKVLPKHSLL |
| | | YEYFTVYNELTKVKYVTEGMRKPAFLSGEQKKAIVDLLFKTNRKVTV |
| | | KQLKEDYFKKIECFDSVEISGVEDRFNASLGTYHDLLKIIKDKDFLDNE |
| | | ENEDILEDIVLTLTLFEDREMIEERLKTYAHLFDDKVMKQLKRRRYTG |
| | | WGRLSRKLINGIRDKQSGKTILDFLKSDGFANRNFMQLIHDDSLTFKED |
| | | IQKAQVSGQGDSLHEHIANLAGSPAIKKGILQTVKVVDELVKVMGRHK |
| | | PENIVIEMARENQTTQKGQKNSRERMKRIEEGIKELGSQILKEHPVENT |
| | | QLQNEKLYLYYLQNGRDMYVDQELDINRLSDYDVDHIVPQSFLKDDSI |
| | | DNKVLTRSDKNRGKSDNVPSEEVVKKMKNYWRQLLNAKLITQRKFD |
| | | NLTKAERGGLSELDKAGFIKRQLVETRQITKHVAQILDSRMNTKYDEN |
| | | DKLIREVKVITLKSKLVSDFRKDFQFYKVREINNYHHAHDAYLNAVVG |
| | | TALIKKYPKLESEFVYGDYKVYDVRKMIAKSEQEIGKATAKYFFYSNI |
| | | MNFFKTEITLANGEIRKRPLIETNGETGEIVWDKGRDFATVRKVLSMPQ |
| | | VNIVKKTEVQTGGFSKESILPKRNSDKLIARKKDWDPKKYGGFDSPTV |
| | | AYSVLVVAKVEKGKSKKLKSVKELLGITIMERSSFEKNPIDFLEAKGYK |
| | | EVKKDLIIKLPKYSLFELENGRKRMLASAGELQKGNELALPSKYVNFL |
| | | YLASHYEKLKGSPEDNEQKQLFVEQHKHYLDEIIEQISEFSKRVILADA |
| | | NLDKVLSAYNKHRDKPIREQAENIIHLFTLTNLGAPAAFKYFDTTIDRK |
| | | RYTSTKEVLDATLIHQSITGLYETRIDLSQLGGDGGGSPKKKRKV |
| 149 | amino acid | MAAFKPNPINYILGLAIGIASVGWAMVEIDEEENPIRLIDLGVRVFERAE |
| | sequence for | VPKTGDSLAMARRLARSVRRLTRRRAHRLLRARRLLKREGVLQAADF |
| | D16A Nme2Cas9 | DENGLIKSLPNTPWQLRAAALDRKLTPLEWSAVLLHLIKHRGYLSQRK |
| | nickase | NEGETADKELGALLKGVANNAHALQTGDFRTPAELALNKFEKESGHI |
| | | RNQRGDYSHTFSRKDLQAELILLFEKQKEFGNPHVSGGLKEGIETLLMT |
| | | QRPALSGDAVQKMLGHCTFEPAEPKAAKNTYTAERFIWLTKLNNLRIL |
| | | EQGSERPLTDTERATLMDEPYRKSKLTYAQARKLLGLEDTAFFKGLRY |
| | | GKDNAEASTLMEMKAYHAISRALEKEGLKDKKSPLNLSSELQDEIGTA |
| | | FSLFKTDEDITGRLKDRVQPEILEALLKHISFDKFVQISLKALRRIVPLME |
| | | QGKRYDEACAEIYGDHYGKKNTEEKIYLPPIPADEIRNPVVLRALSQAR |
| | | KVINGVVRRYGSPARIHIETAREVGKSFKDRKEIEKRQEENRKDREKA |
| | | AAKFREYFPNFVGEPKSKDILKLRLYEQQHGKCLYSGKEINLVRLNEK |
| | | GYVEIDHALPFSRTWDDSFNNKVLVLGSENQNKGNQTPYEYFNGKDN |
| | | SREWQEFKARVETSRFPRSKKQRILLQKFDEDGFKECNLNDTRYVNRF |
| | | LCQFVADHILLTGKGKRRVFASNGQITNLLRGFWGLRKVRAENDRHH |
| | | ALDAVVVACSTVAMQQKITRFVRYKEMNAFDGKTIDKETGKVLHQK |
| | | THFPQPWEFFAQEVMIRVFGKPDGKPEFEEADTPEKLRTLLAEKLSSRP |
| | | EAVHEYVTPLFVSRAPNRKMSGAHKDTLRSAKRFVKHNEKISVKRVW |
| | | LTEIKLADLENMVNYKNGREIELYEALKARLEAYGGNAKQAFDPKDN |
| | | PFYKKGGQLVKAVRVEKTQESGVLLNKKNAYTIADNGDMVRVDVFC |
| | | KVDKKGKNQYFIVPIYAWQVAENILPDIDCKGYRIDDSYTFCFSLHKY |
| | | DLIAFQKDEKSKVEFAYYINCDSSNGRFYLAWHDKGSKEQQFRISTQN |
| | | LVLIQKYQVNELGKEIRPCRLKKRPPVR |
| 150 | coding sequence | GCTGCTTTTAAGCCTAATCCTATTAATTATATTCTTGGTCTTGCTATT |
| | for D16A | GGTATTGCTTCTGTTGGTTGGGCTATGGTTGAGATTGATGAGGAGG |
| | Nme2Cas9 | AGAATCCTATTCGTCTTATTGATCTTGGTGTTCGTGTTTTTGAGCGT |
| | nickase | GCTGAGGTTCCTAAGACTGGTGATTCTCTTGCTATGGCTCGTCGTCT |
| | | TGCTCGTTCTGTTCGTCGTCTTACTCGTCGTCGTGCTCATCGTCTTCT |
| | | TCGTGCTCGTCGTCTTCTTAAGCGTGAGGGTGTTCTTCAGGCTGCTG |
| | | ATTTTGATGAGAATGGTCTTATTAAGTCTCTTCCTAATACTCCTTGG |
| | | CAGCTTCGTGCTGCTGCTCTTGATCGTAAGCTTACTCCTCTTGAGTG |
| | | GTCTGCTGTTCTTCTTCATCTTATTAAGCATCGTGGTTATCTTTCTCA |
| | | GCGTAAGAATGAGGGTGAGACTGCTGATAAGGAGCTTGGTGCTCTT |
| | | CTTAAGGGTGTTGCTAATAATGCTCATGCTCTTCAGACTGGTGATTT |
| | | TCGTACTCCTGCTGAGCTTGCTCTTAATAAGTTTGAGAAGGAGTCTG |
| | | GTCATATTCGTAATCAGCGTGGTGATTATTCTCATACTTTTTCTCGT |
| | | AAGGATCTTCAGGCTGAGCTTATTCTTCTTTTTGAGAAGCAGAAGG |
| | | AGTTTGGTAATCCTCATGTTTCTGGTGGTCTTAAGGAGGGTATTGAG |
| | | ACTCTTCTTATGACTCAGCGTCCTGCTCTTTCTGGTGATGCTGTTCA |
| | | GAAGATGCTTGGTCATTGTACTTTTGAGCCTGCTGAGCCTAAGGCTG |
| | | CTAAGAATACTTATACTGCTGAGCGTTTTATTTGGCTTACTAAGCTT |
| | | AATAATCTTCGTATTCTTGAGCAGGGTTCTGAGCGTCCTCTTACTGA |
| | | TACTGAGCGTGCTACTCTTATGGATGAGCCTTATCGTAAGTCTAAGC |
| | | TTACTTATGCTCAGGCTCGTAAGCTTCTTGGTCTTGAGGATACTGCT |
| | | TTTTTTAAGGGTCTTCGTTATGGTAAGGATAATGCTGAGGCTTCTAC |
| | | TCTTATGGAGATGAAGGCTTATCATGCTATTTCTCGTGCTCTTGAGA |
| | | AGGAGGGTCTTAAGGATAAGAAGTCTCCTCTTAATCTTTCTTCTGAG |
| | | CTTCAGGATGAGATTGGTACTGCTTTTTCTCTTTTTAAGACTGATGA |
| | | GGATATTACTGGTCGTCTTAAGGATCGTGTTCAGCCTGAGATTCTTG |
| | | AGGCTCTTCTTAAGCATATTTCTTTTGATAAGTTTGTTCAGATTTCTC |
| | | TTAAGGCTCTTCGTCGTATTGTTCCTCTTATGGAGCAGGGTAAGCGT |
| | | TATGATGAGGCTTGTGCTGAGATTTATGGTGATCATTATGGTAAGA |
| | | AGAATACTGAGGAGAAGATTTATCTTCCTCCTATTCCTGCTGATGAG |
| | | ATTCGTAATCCTGTTGTTCTTCGTGCTCTTTCTCAGGCTCGTAAGGTT |
| | | ATTAATGGTGTTGTTCGTCGTTATGGTTCTCCTGCTCGTATTCATATT |
| | | GAGACTGCTCGTGAGGTTGGTAAGTCTTTTAAGGATCGTAAGGAGA |
| | | TTGAGAAGCGTCAGGAGGAGAATCGTAAGGATCGTGAGAAGGCTG |
| | | CTGCTAAGTTTCGTGAGTATTTTCCTAATTTTGTTGGTGAGCCTAAG |
| | | TCTAAGGATATTCTTAAGCTTCGTCTTTATGAGCAGCAGCATGGTAA |
| | | GTGTCTTTATTCTGGTAAGGAGATTAATCTTGTTCGTCTTAATGAGA |
| | | AGGGTTATGTTGAGATTGATCATGCTCTTCCTTTTTCTCGTACTTGG |
| | | GATGATTCTTTTAATAATAAGGTTCTTGTTCTTGGTTCTGAGAATCA |
| | | GAATAAGGGTAATCAGACTCCTTATGAGTATTTTAATGGTAAGGAT |
| | | AATTCTCGTGAGTGGCAGGAGTTTAAGGCTCGTGTTGAGACTTCTC |
| | | GTTTTCCTCGTTCTAAGAAGCAGCGTATTCTTCTTCAGAAGTTTGAT |
| | | GAGGATGGTTTTAAGGAGTGTAATCTTAATGATACTCGTTATGTTAA |
| | | TCGTTTTCTTTGTCAGTTTGTTGCTGATCATATTCTTCTTACTGGTAA |
| | | GGGTAAGCGTCGTGTTTTTGCTTCTAATGGTCAGATTACTAATCTTC |
| | | TTCGTGGTTTTTGGGGTCTTCGTAAGGTTCGTGCTGAGAATGATCGT |
| | | CATCATGCTCTTGATGCTGTTGTTGTTGCTTGTTCTACTGTTGCTATG |
| | | CAGCAGAAGATTACTCGTTTTGTTCGTTATAAGGAGATGAATGCTTT |
| | | TGATGGTAAGACTATTGATAAGGAGACTGGTAAGGTTCTTCATCAG |
| | | AAGACTCATTTTCCTCAGCCTTGGGAGTTTTTTGCTCAGGAGGTTAT |
| | | GATTCGTGTTTTTGGTAAGCCTGATGGTAAGCCTGAGTTTGAGGAG |
| | | GCTGATACTCCTGAGAAGCTTCGTACTCTTCTTGCTGAGAAGCTTTC |
| | | TTCTCGTCCTGAGGCTGTTCATGAGTATGTTACTCCTCTTTTTGTTTC |
| | | TCGTGCTCCTAATCGTAAGATGTCTGGTGCTCATAAGGATACTCTTC |
| | | GTTCTGCTAAGCGTTTTGTTAAGCATAATGAGAAGATTTCTGTTAAG |
| | | CGTGTTTGGCTTACTGAGATTAAGCTTGCTGATCTTGAGAATATGGT |
| | | TAATTATAAGAATGGTCGTGAGATTGAGCTTTATGAGGCTCTTAAG |
| | | GCTCGTCTTGAGGCTTATGGTGGTAATGCTAAGCAGGCTTTTGATCC |
| | | TAAGGATAATCCTTTTTATAAGAAGGGTGGTCAGCTTGTTAAGGCT |
| | | GTTCGTGTTGAGAAGACTCAGGAGTCTGGTGTTCTTCTTAATAAGA |
| | | AGAATGCTTATACTATTGCTGATAATGGTGATATGGTTCGTGTTGAT |
| | | GTTTTTTGTAAGGTTGATAAGAAGGGTAAGAATCAGTATTTTATTGT |
| | | TCCTATTTATGCTTGGCAGGTTGCTGAGAATATTCTTCCTGATATTG |
| | | ATTGTAAGGGTTATCGTATTGATGATTCTTATACTTTTTGTTTTTCTC |
| | | TTCATAAGTATGATCTTATTGCTTTTCAGAAGGATGAGAAGTCTAAG |
| | | GTTGAGTTTGCTTATTATATTAATTGTGATTCTTCTAATGGTCGTTTT |
| | | TATCTTGCTTGGCATGATAAGGGTTCTAAGGAGCAGCAGTTTCGTAT |
| | | TTCTACTCAGAATCTTGTTCTTATTCAGAAGTATCAGGTTAATGAGC |
| | | TTGGTAAGGAGATTCGTCCTTGTCGTCTTAAGAAGCGTCCTCCTGTT |
| | | CGT |
| 151 | coding sequence | GCCGCCTTCAAGCCCAACCCCATCAACTACATCCTGGGCCTGGCCA |
| | for D16A | TCGGCATCGCCTCCGTGGGCTGGGCCATGGTGGAGATCGACGAGGA |
| | Nme2Cas9 | GGAGAACCCCATCCGGCTGATCGACCTGGGCGTGCGGGTGTTCGAG |
| | nickase | CGGGCCGAGGTGCCCAAGACCGGCGACTCCCTGGCCATGGCCCGGC |
| | | GGCTGGCCCGGTCCGTGCGGCGGCTGACCCGGCGGCGGGCCCACCG |
| | | GCTGCTGCGGGCCCGGCGGCTGCTGAAGCGGGAGGGCGTGCTGCA |
| | | GGCCGCCGACTTCGACGAGAACGGCCTGATCAAGTCCCTGCCCAAC |
| | | ACCCCCTGGCAGCTGCGGGCCGCCGCCCTGGACCGGAAGCTGACCC |
| | | CCCTGGAGTGGTCCGCCGTGCTGCTGCACCTGATCAAGCACCGGGG |
| | | CTACCTGTCCCAGCGGAAGAACGAGGGCGAGACCGCCGACAAGGA |
| | | GCTGGGCGCCCTGCTGAAGGGCGTGGCCAACAACGCCCACGCCCTG |
| | | CAGACCGGCGACTTCCGGACCCCCGCCGAGCTGGCCCTGAACAAGT |
| | | TCGAGAAGGAGTCCGGCCACATCCGGAACCAGCGGGGCGACTACT |
| | | CCCACACCTTCTCCCGGAAGGACCTGCAGGCCGAGCTGATCCTGCT |
| | | GTTCGAGAAGCAGAAGGAGTTCGGCAACCCCCACGTGTCCGGCGGC |
| | | CTGAAGGAGGGCATCGAGACCCTGCTGATGACCCAGCGGCCCGCCC |
| | | TGTCCGGCGACGCCGTGCAGAAGATGCTGGGCCACTGCACCTTCGA |
| | | GCCCGCCGAGCCCAAGGCCGCCAAGAACACCTACACCGCCGAGCG |
| | | GTTCATCTGGCTGACCAAGCTGAACAACCTGCGGATCCTGGAGCAG |
| | | GGCTCCGAGCGGCCCCTGACCGACACCGAGCGGGCCACCCTGATGG |
| | | ACGAGCCCTACCGGAAGTCCAAGCTGACCTACGCCCAGGCCCGGAA |
| | | GCTGCTGGGCCTGGAGGACACCGCCTTCTTCAAGGGCCTGCGGTAC |
| | | GGCAAGGACAACGCCGAGGCCTCCACCCTGATGGAGATGAAGGCC |
| | | TACCACGCCATCTCCCGGGCCCTGGAGAAGGAGGGCCTGAAGGAC |
| | | AAGAAGTCCCCCCTGAACCTGTCCTCCGAGCTGCAGGACGAGATCG |
| | | GCACCGCCTTCTCCCTGTTCAAGACCGACGAGGACATCACCGGCCG |
| | | GCTGAAGGACCGGGTGCAGCCCGAGATCCTGGAGGCCCTGCTGAA |
| | | GCACATCTCCTTCGACAAGTTCGTGCAGATCTCCCTGAAGGCCCTGC |
| | | GGCGGATCGTGCCCCTGATGGAGCAGGGCAAGCGGTACGACGAGG |
| | | CCTGCGCCGAGATCTACGGCGACCACTACGGCAAGAAGAACACCG |
| | | AGGAGAAGATCTACCTGCCCCCCATCCCCGCCGACGAGATCCGGAA |
| | | CCCCGTGGTGCTGCGGGCCCTGTCCCAGGCCCGGAAGGTGATCAAC |
| | | GGCGTGGTGCGGCGGTACGGCTCCCCCGCCCGGATCCACATCGAGA |
| | | CCGCCCGGGAGGTGGGCAAGTCCTTCAAGGACCGGAAGGAGATCG |
| | | AGAAGCGGCAGGAGGAGAACCGGAAGGACCGGGAGAAGGCCGCC |
| | | GCCAAGTTCCGGGAGTACTTCCCCAACTTCGTGGGCGAGCCCAAGT |
| | | CCAAGGACATCCTGAAGCTGCGGCTGTACGAGCAGCAGCACGGCA |
| | | AGTGCCTGTACTCCGGCAAGGAGATCAACCTGGTGCGGCTGAACGA |
| | | GAAGGGCTACGTGGAGATCGACCACGCCCTGCCCTTCTCCCGGACC |
| | | TGGGACGACTCCTTCAACAACAAGGTGCTGGTGCTGGGCTCCGAGA |
| | | ACCAGAACAAGGGCAACCAGACCCCCTACGAGTACTTCAACGGCA |
| | | AGGACAACTCCCGGGAGTGGCAGGAGTTCAAGGCCCGGGTGGAGA |
| | | CCTCCCGGTTCCCCCGGTCCAAGAAGCAGCGGATCCTGCTGCAGAA |
| | | GTTCGACGAGGACGGCTTCAAGGAGTGCAACCTGAACGACACCCG |
| | | GTACGTGAACCGGTTCCTGTGCCAGTTCGTGGCCGACCACATCCTG |
| | | CTGACCGGCAAGGGCAAGCGGCGGGTGTTCGCCTCCAACGGCCAG |
| | | ATCACCAACCTGCTGCGGGGCTTCTGGGGCCTGCGGAAGGTGCGGG |
| | | CCGAGAACGACCGGCACCACGCCCTGGACGCCGTGGTGGTGGCCTG |
| | | CTCCACCGTGGCCATGCAGCAGAAGATCACCCGGTTCGTGCGGTAC |
| | | AAGGAGATGAACGCCTTCGACGGCAAGACCATCGACAAGGAGACC |
| | | GGCAAGGTGCTGCACCAGAAGACCCACTTCCCCCAGCCCTGGGAGT |
| | | TCTTCGCCCAGGAGGTGATGATCCGGGTGTTCGGCAAGCCCGACGG |
| | | CAAGCCCGAGTTCGAGGAGGCCGACACCCCCGAGAAGCTGCGGAC |
| | | CCTGCTGGCCGAGAAGCTGTCCTCCCGGCCCGAGGCCGTGCACGAG |
| | | TACGTGACCCCCCTGTTCGTGTCCCGGGCCCCCAACCGGAAGATGT |
| | | CCGGCGCCCACAAGGACACCCTGCGGTCCGCCAAGCGGTTCGTGAA |
| | | GCACAACGAGAAGATCTCCGTGAAGCGGGTGTGGCTGACCGAGAT |
| | | CAAGCTGGCCGACCTGGAGAACATGGTGAACTACAAGAACGGCCG |
| | | GGAGATCGAGCTGTACGAGGCCCTGAAGGCCCGGCTGGAGGCCTA |
| | | CGGCGGCAACGCCAAGCAGGCCTTCGACCCCAAGGACAACCCCTTC |
| | | TACAAGAAGGGCGGCCAGCTGGTGAAGGCCGTGCGGGTGGAGAAG |
| | | ACCCAGGAGTCCGGCGTGCTGCTGAACAAGAAGAACGCCTACACC |
| | | ATCGCCGACAACGGCGACATGGTGCGGGTGGACGTGTTCTGCAAGG |
| | | TGGACAAGAAGGGCAAGAACCAGTACTTCATCGTGCCCATCTACGC |
| | | CTGGCAGGTGGCCGAGAACATCCTGCCCGACATCGACTGCAAGGGC |
| | | TACCGGATCGACGACTCCTACACCTTCTGCTTCTCCCTGCACAAGTA |
| | | CGACCTGATCGCCTTCCAGAAGGACGAGAAGTCCAAGGTGGAGTTC |
| | | GCCTACTACATCAACTGCGACTCCTCCAACGGCCGGTTCTACCTGGC |
| | | CTGGCACGACAAGGGCTCCAAGGAGCAGCAGTTCCGGATCTCCACC |
| | | CAGAACCTGGTGCTGATCCAGAAGTACCAGGTGAACGAGCTGGGC |
| | | AAGGAGATCCGGCCCTGCCGGCTGAAGAAGCGGCCCCCCGTGCGG |
| 152 | coding sequence | GCCGCCTTCAAGCCCAACCCCATCAACTACATCCTGGGCCTGGCCA |
| | for D16A | TCGGCATCGCCTCCGTGGGCTGGGCCATGGTGGAGATCGACGAGGA |
| | Nme2Cas9 | GGAGAACCCCATCCGGCTGATCGACCTGGGCGTGCGGGTGTTCGAG |
| | nickase | CGGGCCGAGGTGCCCAAGACCGGCGACTCCCTGGCCATGGCCCGGC |
| | | GGCTGGCCCGGTCCGTGCGGCGGCTGACCCGGCGGCGGGCCCACCG |
| | | GCTGCTGCGGGCCCGGCGGCTGCTGAAGCGGGAGGGCGTGCTGCA |
| | | GGCCGCCGACTTCGACGAGAACGGCCTGATCAAGTCCCTGCCCAAC |
| | | ACCCCCTGGCAGCTGCGGGCCGCCGCCCTGGACCGGAAGCTGACCC |
| | | CCCTGGAGTGGTCCGCCGTGCTGCTGCACCTGATCAAGCACCGGGG |
| | | CTACCTGTCCCAGCGGAAGAACGAGGGCGAGACCGCCGACAAGGA |
| | | GCTGGGCGCCCTGCTGAAGGGCGTGGCCAACAACGCCCACGCCCTG |
| | | CAGACCGGCGACTTCCGGACCCCCGCCGAGCTGGCCCTGAACAAGT |
| | | TCGAGAAGGAGTCCGGCCACATCCGGAACCAGCGGGGCGACTACT |
| | | CCCACACCTTCTCCCGGAAGGACCTGCAGGCCGAGCTGATCCTGCT |
| | | GTTCGAGAAGCAGAAGGAGTTCGGCAACCCCCACGTGTCCGGCGGC |
| | | CTGAAGGAGGGCATCGAGACCCTGCTGATGACCCAGCGGCCCGCCC |
| | | TGTCCGGCGACGCCGTGCAGAAGATGCTGGGCCACTGCACCTTCGA |
| | | GCCCGCCGAGCCCAAGGCCGCCAAGAACACCTACACCGCCGAGCG |
| | | GTTCATCTGGCTGACCAAGCTGAACAACCTGCGGATCCTGGAGCAG |
| | | GGCTCCGAGCGGCCCCTGACCGACACCGAGCGGGCCACCCTGATGG |
| | | ACGAGCCCTACCGGAAGTCCAAGCTGACCTACGCCCAGGCCCGGAA |
| | | GCTGCTGGGCCTGGAGGACACCGCCTTCTTCAAGGGCCTGCGGTAC |
| | | GGCAAGGACAACGCCGAGGCCTCCACCCTGATGGAGATGAAGGCC |
| | | TACCACGCCATCTCCCGGGCCCTGGAGAAGGAGGGCCTGAAGGAC |
| | | AAGAAGTCCCCCCTGAACCTGTCCTCCGAGCTGCAGGACGAGATCG |
| | | GCACCGCCTTCTCCCTGTTCAAGACCGACGAGGACATCACCGGCCG |
| | | GCTGAAGGACCGGGTGCAGCCCGAGATCCTGGAGGCCCTGCTGAA |
| | | GCACATCTCCTTCGACAAGTTCGTGCAGATCTCCCTGAAGGCCCTGC |
| | | GGCGGATCGTGCCCCTGATGGAGCAGGGCAAGCGGTACGACGAGG |
| | | CCTGCGCCGAGATCTACGGCGACCACTACGGCAAGAAGAACACCG |
| | | AGGAGAAGATCTACCTGCCCCCCATCCCCGCCGACGAGATCCGGAA |
| | | CCCCGTGGTGCTGCGGGCCCTGTCCCAGGCCCGGAAGGTGATCAAC |
| | | GGCGTGGTGCGGCGGTACGGCTCCCCCGCCCGGATCCACATCGAGA |
| | | CCGCCCGGGAGGTGGGCAAGTCCTTCAAGGACCGGAAGGAGATCG |
| | | AGAAGCGGCAGGAGGAGAACCGGAAGGACCGGGAGAAGGCCGCC |
| | | GCCAAGTTCCGGGAGTACTTCCCCAACTTCGTGGGCGAGCCCAAGT |
| | | CCAAGGACATCCTGAAGCTGCGGCTGTACGAGCAGCAGCACGGCA |
| | | AGTGCCTGTACTCCGGCAAGGAGATCAACCTGGTGCGGCTGAACGA |
| | | GAAGGGCTACGTGGAGATCGACCACGCCCTGCCCTTCTCCCGGACC |
| | | TGGGACGACTCCTTCAACAACAAGGTGCTGGTGCTGGGCTCCGAGA |
| | | ACCAGAACAAGGGCAACCAGACCCCCTACGAGTACTTCAACGGCA |
| | | AGGACAACTCCCGGGAGTGGCAGGAGTTCAAGGCCCGGGTGGAGA |
| | | CCTCCCGGTTCCCCCGGTCCAAGAAGCAGCGGATCCTGCTGCAGAA |
| | | GTTCGACGAGGACGGCTTCAAGGAGTGCAACCTGAACGACACCCG |
| | | GTACGTGAACCGGTTCCTGTGCCAGTTCGTGGCCGACCACATCCTG |
| | | CTGACCGGCAAGGGCAAGCGGCGGGTGTTCGCCTCCAACGGCCAG |
| | | ATCACCAACCTGCTGCGGGGCTTCTGGGGCCTGCGGAAGGTGCGGG |
| | | CCGAGAACGACCGGCACCACGCCCTGGACGCCGTGGTGGTGGCCTG |
| | | CTCCACCGTGGCCATGCAGCAGAAGATCACCCGGTTCGTGCGGTAC |
| | | AAGGAGATGAACGCCTTCGACGGCAAGACCATCGACAAGGAGACC |
| | | GGCAAGGTGCTGCACCAGAAGACCCACTTCCCCCAGCCCTGGGAGT |
| | | TCTTCGCCCAGGAGGTGATGATCCGGGTGTTCGGCAAGCCCGACGG |
| | | CAAGCCCGAGTTCGAGGAGGCCGACACCCCCGAGAAGCTGCGGAC |
| | | CCTGCTGGCCGAGAAGCTGTCCTCCCGGCCCGAGGCCGTGCACGAG |
| | | TACGTGACCCCCCTGTTCGTGTCCCGGGCCCCCAACCGGAAGATGT |
| | | CCGGCGCCCACAAGGACACCCTGCGGTCCGCCAAGCGGTTCGTGAA |
| | | GCACAACGAGAAGATCTCCGTGAAGCGGGTGTGGCTGACCGAGAT |
| | | CAAGCTGGCCGACCTGGAGAACATGGTGAACTACAAGAACGGCCG |
| | | GGAGATCGAGCTGTACGAGGCCCTGAAGGCCCGGCTGGAGGCCTA |
| | | CGGCGGCAACGCCAAGCAGGCCTTCGACCCCAAGGACAACCCCTTC |
| | | TACAAGAAGGGCGGCCAGCTGGTGAAGGCCGTGCGGGTGGAGAAG |
| | | ACCCAGGAGTCCGGCGTGCTGCTGAACAAGAAGAACGCCTACACC |
| | | ATCGCCGACAACGGCGACATGGTGCGGGTGGACGTGTTCTGCAAGG |
| | | TGGACAAGAAGGGCAAGAACCAGTACTTCATCGTGCCCATCTACGC |
| | | CTGGCAGGTGGCCGAGAACATCCTGCCCGACATCGACTGCAAGGGC |
| | | TACCGGATCGACGACTCCTACACCTTCTGCTTCTCCCTGCACAAGTA |
| | | CGACCTGATCGCCTTCCAGAAGGACGAGAAGTCCAAGGTGGAGTTC |
| | | GCCTACTACATCAACTGCGACTCCTCCAACGGCCGGTTCTACCTGGC |
| | | CTGGCACGACAAGGGCTCCAAGGAGCAGCAGTTCCGGATCTCCACC |
| | | CAGAACCTGGTGCTGATCCAGAAGTACCAGGTGAACGAGCTGGGC |
| | | AAGGAGATCCGGCCCTGCCGGCTGAAGAAGCGGCCCCCCGTGCGG |
| 153 | open reading | ATGGCTGCTTTTAAGCCTAATCCTATTAATTATATTCTTGGTCTTGCT |
| | frame for D16A | ATTGGTATTGCTTCTGTTGGTTGGGCTATGGTTGAGATTGATGAGGA |
| | Nme2Cas9 | GGAGAATCCTATTCGTCTTATTGATCTTGGTGTTCGTGTTTTTGAGC |
| | nickase | GTGCTGAGGTTCCTAAGACTGGTGATTCTCTTGCTATGGCTCGTCGT |
| | | CTTGCTCGTTCTGTTCGTCGTCTTACTCGTCGTCGTGCTCATCGTCTT |
| | | CTTCGTGCTCGTCGTCTTCTTAAGCGTGAGGGTGTTCTTCAGGCTGC |
| | | TGATTTTGATGAGAATGGTCTTATTAAGTCTCTTCCTAATACTCCTT |
| | | GGCAGCTTCGTGCTGCTGCTCTTGATCGTAAGCTTACTCCTCTTGAG |
| | | TGGTCTGCTGTTCTTCTTCATCTTATTAAGCATCGTGGTTATCTTTCT |
| | | CAGCGTAAGAATGAGGGTGAGACTGCTGATAAGGAGCTTGGTGCTC |
| | | TTCTTAAGGGTGTTGCTAATAATGCTCATGCTCTTCAGACTGGTGAT |
| | | TTTCGTACTCCTGCTGAGCTTGCTCTTAATAAGTTTGAGAAGGAGTC |
| | | TGGTCATATTCGTAATCAGCGTGGTGATTATTCTCATACTTTTTCTC |
| | | GTAAGGATCTTCAGGCTGAGCTTATTCTTCTTTTTGAGAAGCAGAAG |
| | | GAGTTTGGTAATCCTCATGTTTCTGGTGGTCTTAAGGAGGGTATTGA |
| | | GACTCTTCTTATGACTCAGCGTCCTGCTCTTTCTGGTGATGCTGTTC |
| | | AGAAGATGCTTGGTCATTGTACTTTTGAGCCTGCTGAGCCTAAGGCT |
| | | GCTAAGAATACTTATACTGCTGAGCGTTTTATTTGGCTTACTAAGCT |
| | | TAATAATCTTCGTATTCTTGAGCAGGGTTCTGAGCGTCCTCTTACTG |
| | | ATACTGAGCGTGCTACTCTTATGGATGAGCCTTATCGTAAGTCTAAG |
| | | CTTACTTATGCTCAGGCTCGTAAGCTTCTTGGTCTTGAGGATACTGC |
| | | TTTTTTTAAGGGTCTTCGTTATGGTAAGGATAATGCTGAGGCTTCTA |
| | | CTCTTATGGAGATGAAGGCTTATCATGCTATTTCTCGTGCTCTTGAG |
| | | AAGGAGGGTCTTAAGGATAAGAAGTCTCCTCTTAATCTTTCTTCTGA |
| | | GCTTCAGGATGAGATTGGTACTGCTTTTTCTCTTTTTAAGACTGATG |
| | | AGGATATTACTGGTCGTCTTAAGGATCGTGTTCAGCCTGAGATTCTT |
| | | GAGGCTCTTCTTAAGCATATTTCTTTTGATAAGTTTGTTCAGATTTCT |
| | | CTTAAGGCTCTTCGTCGTATTGTTCCTCTTATGGAGCAGGGTAAGCG |
| | | TTATGATGAGGCTTGTGCTGAGATTTATGGTGATCATTATGGTAAGA |
| | | AGAATACTGAGGAGAAGATTTATCTTCCTCCTATTCCTGCTGATGAG |
| | | ATTCGTAATCCTGTTGTTCTTCGTGCTCTTTCTCAGGCTCGTAAGGTT |
| | | ATTAATGGTGTTGTTCGTCGTTATGGTTCTCCTGCTCGTATTCATATT |
| | | GAGACTGCTCGTGAGGTTGGTAAGTCTTTTAAGGATCGTAAGGAGA |
| | | TTGAGAAGCGTCAGGAGGAGAATCGTAAGGATCGTGAGAAGGCTG |
| | | CTGCTAAGTTTCGTGAGTATTTTCCTAATTTTGTTGGTGAGCCTAAG |
| | | TCTAAGGATATTCTTAAGCTTCGTCTTTATGAGCAGCAGCATGGTAA |
| | | GTGTCTTTATTCTGGTAAGGAGATTAATCTTGTTCGTCTTAATGAGA |
| | | AGGGTTATGTTGAGATTGATCATGCTCTTCCTTTTTCTCGTACTTGG |
| | | GATGATTCTTTTAATAATAAGGTTCTTGTTCTTGGTTCTGAGAATCA |
| | | GAATAAGGGTAATCAGACTCCTTATGAGTATTTTAATGGTAAGGAT |
| | | AATTCTCGTGAGTGGCAGGAGTTTAAGGCTCGTGTTGAGACTTCTC |
| | | GTTTTCCTCGTTCTAAGAAGCAGCGTATTCTTCTTCAGAAGTTTGAT |
| | | GAGGATGGTTTTAAGGAGTGTAATCTTAATGATACTCGTTATGTTAA |
| | | TCGTTTTCTTTGTCAGTTTGTTGCTGATCATATTCTTCTTACTGGTAA |
| | | GGGTAAGCGTCGTGTTTTTGCTTCTAATGGTCAGATTACTAATCTTC |
| | | TTCGTGGTTTTTGGGGTCTTCGTAAGGTTCGTGCTGAGAATGATCGT |
| | | CATCATGCTCTTGATGCTGTTGTTGTTGCTTGTTCTACTGTTGCTATG |
| | | CAGCAGAAGATTACTCGTTTTGTTCGTTATAAGGAGATGAATGCTTT |
| | | TGATGGTAAGACTATTGATAAGGAGACTGGTAAGGTTCTTCATCAG |
| | | AAGACTCATTTTCCTCAGCCTTGGGAGTTTTTTGCTCAGGAGGTTAT |
| | | GATTCGTGTTTTTGGTAAGCCTGATGGTAAGCCTGAGTTTGAGGAG |
| | | GCTGATACTCCTGAGAAGCTTCGTACTCTTCTTGCTGAGAAGCTTTC |
| | | TTCTCGTCCTGAGGCTGTTCATGAGTATGTTACTCCTCTTTTTGTTTC |
| | | TCGTGCTCCTAATCGTAAGATGTCTGGTGCTCATAAGGATACTCTTC |
| | | GTTCTGCTAAGCGTTTTGTTAAGCATAATGAGAAGATTTCTGTTAAG |
| | | CGTGTTTGGCTTACTGAGATTAAGCTTGCTGATCTTGAGAATATGGT |
| | | TAATTATAAGAATGGTCGTGAGATTGAGCTTTATGAGGCTCTTAAG |
| | | GCTCGTCTTGAGGCTTATGGTGGTAATGCTAAGCAGGCTTTTGATCC |
| | | TAAGGATAATCCTTTTTATAAGAAGGGTGGTCAGCTTGTTAAGGCT |
| | | GTTCGTGTTGAGAAGACTCAGGAGTCTGGTGTTCTTCTTAATAAGA |
| | | AGAATGCTTATACTATTGCTGATAATGGTGATATGGTTCGTGTTGAT |
| | | GTTTTTTGTAAGGTTGATAAGAAGGGTAAGAATCAGTATTTTATTGT |
| | | TCCTATTTATGCTTGGCAGGTTGCTGAGAATATTCTTCCTGATATTG |
| | | ATTGTAAGGGTTATCGTATTGATGATTCTTATACTTTTTGTTTTTCTC |
| | | TTCATAAGTATGATCTTATTGCTTTTCAGAAGGATGAGAAGTCTAAG |
| | | GTTGAGTTTGCTTATTATATTAATTGTGATTCTTCTAATGGTCGTTTT |
| | | TATCTTGCTTGGCATGATAAGGGTTCTAAGGAGCAGCAGTTTCGTAT |
| | | TTCTACTCAGAATCTTGTTCTTATTCAGAAGTATCAGGTTAATGAGC |
| | | TTGGTAAGGAGATTCGTCCTTGTCGTCTTAAGAAGCGTCCTCCTGTT |
| | | CGTUGA |
| 154 | open reading | ATGGCCGCCTTCAAGCCCAACCCCATCAACTACATCCTGGGCCTGG |
| | frame for D16A | CCATCGGCATCGCCTCCGTGGGCTGGGCCATGGTGGAGATCGACGA |
| | Nme2Cas9 | GGAGGAGAACCCCATCCGGCTGATCGACCTGGGCGTGCGGGTGTTC |
| | nickase | GAGCGGGCCGAGGTGCCCAAGACCGGCGACTCCCTGGCCATGGCCC |
| | | GGCGGCTGGCCCGGTCCGTGCGGCGGCTGACCCGGCGGCGGGCCCA |
| | | CCGGCTGCTGCGGGCCCGGCGGCTGCTGAAGCGGGAGGGCGTGCTG |
| | | CAGGCCGCCGACTTCGACGAGAACGGCCTGATCAAGTCCCTGCCCA |
| | | ACACCCCCTGGCAGCTGCGGGCCGCCGCCCTGGACCGGAAGCTGAC |
| | | CCCCCTGGAGTGGTCCGCCGTGCTGCTGCACCTGATCAAGCACCGG |
| | | GGCTACCTGTCCCAGCGGAAGAACGAGGGCGAGACCGCCGACAAG |
| | | GAGCTGGGCGCCCTGCTGAAGGGCGTGGCCAACAACGCCCACGCCC |
| | | TGCAGACCGGCGACTTCCGGACCCCCGCCGAGCTGGCCCTGAACAA |
| | | GTTCGAGAAGGAGTCCGGCCACATCCGGAACCAGCGGGGCGACTA |
| | | CTCCCACACCTTCTCCCGGAAGGACCTGCAGGCCGAGCTGATCCTG |
| | | CTGTTCGAGAAGCAGAAGGAGTTCGGCAACCCCCACGTGTCCGGCG |
| | | GCCTGAAGGAGGGCATCGAGACCCTGCTGATGACCCAGCGGCCCGC |
| | | CCTGTCCGGCGACGCCGTGCAGAAGATGCTGGGCCACTGCACCTTC |
| | | GAGCCCGCCGAGCCCAAGGCCGCCAAGAACACCTACACCGCCGAG |
| | | CGGTTCATCTGGCTGACCAAGCTGAACAACCTGCGGATCCTGGAGC |
| | | AGGGCTCCGAGCGGCCCCTGACCGACACCGAGCGGGCCACCCTGAT |
| | | GGACGAGCCCTACCGGAAGTCCAAGCTGACCTACGCCCAGGCCCGG |
| | | AAGCTGCTGGGCCTGGAGGACACCGCCTTCTTCAAGGGCCTGCGGT |
| | | ACGGCAAGGACAACGCCGAGGCCTCCACCCTGATGGAGATGAAGG |
| | | CCTACCACGCCATCTCCCGGGCCCTGGAGAAGGAGGGCCTGAAGGA |
| | | CAAGAAGTCCCCCCTGAACCTGTCCTCCGAGCTGCAGGACGAGATC |
| | | GGCACCGCCTTCTCCCTGTTCAAGACCGACGAGGACATCACCGGCC |
| | | GGCTGAAGGACCGGGTGCAGCCCGAGATCCTGGAGGCCCTGCTGA |
| | | AGCACATCTCCTTCGACAAGTTCGTGCAGATCTCCCTGAAGGCCCT |
| | | GCGGCGGATCGTGCCCCTGATGGAGCAGGGCAAGCGGTACGACGA |
| | | GGCCTGCGCCGAGATCTACGGCGACCACTACGGCAAGAAGAACAC |
| | | CGAGGAGAAGATCTACCTGCCCCCCATCCCCGCCGACGAGATCCGG |
| | | AACCCCGTGGTGCTGCGGGCCCTGTCCCAGGCCCGGAAGGTGATCA |
| | | ACGGCGTGGTGCGGCGGTACGGCTCCCCCGCCCGGATCCACATCGA |
| | | GACCGCCCGGGAGGTGGGCAAGTCCTTCAAGGACCGGAAGGAGAT |
| | | CGAGAAGCGGCAGGAGGAGAACCGGAAGGACCGGGAGAAGGCCG |
| | | CCGCCAAGTTCCGGGAGTACTTCCCCAACTTCGTGGGCGAGCCCAA |
| | | GTCCAAGGACATCCTGAAGCTGCGGCTGTACGAGCAGCAGCACGGC |
| | | AAGTGCCTGTACTCCGGCAAGGAGATCAACCTGGTGCGGCTGAACG |
| | | AGAAGGGCTACGTGGAGATCGACCACGCCCTGCCCTTCTCCCGGAC |
| | | CTGGGACGACTCCTTCAACAACAAGGTGCTGGTGCTGGGCTCCGAG |
| | | AACCAGAACAAGGGCAACCAGACCCCCTACGAGTACTTCAACGGC |
| | | AAGGACAACTCCCGGGAGTGGCAGGAGTTCAAGGCCCGGGTGGAG |
| | | ACCTCCCGGTTCCCCCGGTCCAAGAAGCAGCGGATCCTGCTGCAGA |
| | | AGTTCGACGAGGACGGCTTCAAGGAGTGCAACCTGAACGACACCC |
| | | GGTACGTGAACCGGTTCCTGTGCCAGTTCGTGGCCGACCACATCCT |
| | | GCTGACCGGCAAGGGCAAGCGGCGGGTGTTCGCCTCCAACGGCCA |
| | | GATCACCAACCTGCTGCGGGGCTTCTGGGGCCTGCGGAAGGTGCGG |
| | | GCCGAGAACGACCGGCACCACGCCCTGGACGCCGTGGTGGTGGCCT |
| | | GCTCCACCGTGGCCATGCAGCAGAAGATCACCCGGTTCGTGCGGTA |
| | | CAAGGAGATGAACGCCTTCGACGGCAAGACCATCGACAAGGAGAC |
| | | CGGCAAGGTGCTGCACCAGAAGACCCACTTCCCCCAGCCCTGGGAG |
| | | TTCTTCGCCCAGGAGGTGATGATCCGGGTGTTCGGCAAGCCCGACG |
| | | GCAAGCCCGAGTTCGAGGAGGCCGACACCCCCGAGAAGCTGCGGA |
| | | CCCTGCTGGCCGAGAAGCTGTCCTCCCGGCCCGAGGCCGTGCACGA |
| | | GTACGTGACCCCCCTGTTCGTGTCCCGGGCCCCCAACCGGAAGATG |
| | | TCCGGCGCCCACAAGGACACCCTGCGGTCCGCCAAGCGGTTCGTGA |
| | | AGCACAACGAGAAGATCTCCGTGAAGCGGGTGTGGCTGACCGAGA |
| | | TCAAGCTGGCCGACCTGGAGAACATGGTGAACTACAAGAACGGCC |
| | | GGGAGATCGAGCTGTACGAGGCCCTGAAGGCCCGGCTGGAGGCCT |
| | | ACGGCGGCAACGCCAAGCAGGCCTTCGACCCCAAGGACAACCCCTT |
| | | CTACAAGAAGGGCGGCCAGCTGGTGAAGGCCGTGCGGGTGGAGAA |
| | | GACCCAGGAGTCCGGCGTGCTGCTGAACAAGAAGAACGCCTACAC |
| | | CATCGCCGACAACGGCGACATGGTGCGGGTGGACGTGTTCTGCAAG |
| | | GTGGACAAGAAGGGCAAGAACCAGTACTTCATCGTGCCCATCTACG |
| | | CCTGGCAGGTGGCCGAGAACATCCTGCCCGACATCGACTGCAAGGG |
| | | CTACCGGATCGACGACTCCTACACCTTCTGCTTCTCCCTGCACAAGT |
| | | ACGACCTGATCGCCTTCCAGAAGGACGAGAAGTCCAAGGTGGAGTT |
| | | CGCCTACTACATCAACTGCGACTCCTCCAACGGCCGGTTCTACCTGG |
| | | CCTGGCACGACAAGGGCTCCAAGGAGCAGCAGTTCCGGATCTCCAC |
| | | CCAGAACCTGGTGCTGATCCAGAAGTACCAGGTGAACGAGCTGGGC |
| | | AAGGAGATCCGGCCCTGCCGGCTGAAGAAGCGGCCCCCCGTGCGG |
| | | UGA |
| 155 | open reading | ATGGCCGCCTTCAAGCCCAACCCCATCAACTACATCCTGGGCCTGG |
| | frame for D16A | CCATCGGCATCGCCTCCGTGGGCTGGGCCATGGTGGAGATCGACGA |
| | Nme2Cas9 | GGAGGAGAACCCCATCCGGCTGATCGACCTGGGCGTGCGGGTGTTC |
| | nickase | GAGCGGGCCGAGGTGCCCAAGACCGGCGACTCCCTGGCCATGGCCC |
| | | GGCGGCTGGCCCGGTCCGTGCGGCGGCTGACCCGGCGGCGGGCCCA |
| | | CCGGCTGCTGCGGGCCCGGCGGCTGCTGAAGCGGGAGGGCGTGCTG |
| | | CAGGCCGCCGACTTCGACGAGAACGGCCTGATCAAGTCCCTGCCCA |
| | | ACACCCCCTGGCAGCTGCGGGCCGCCGCCCTGGACCGGAAGCTGAC |
| | | CCCCCTGGAGTGGTCCGCCGTGCTGCTGCACCTGATCAAGCACCGG |
| | | GGCTACCTGTCCCAGCGGAAGAACGAGGGCGAGACCGCCGACAAG |
| | | GAGCTGGGCGCCCTGCTGAAGGGCGTGGCCAACAACGCCCACGCCC |
| | | TGCAGACCGGCGACTTCCGGACCCCCGCCGAGCTGGCCCTGAACAA |
| | | GTTCGAGAAGGAGTCCGGCCACATCCGGAACCAGCGGGGCGACTA |
| | | CTCCCACACCTTCTCCCGGAAGGACCTGCAGGCCGAGCTGATCCTG |
| | | CTGTTCGAGAAGCAGAAGGAGTTCGGCAACCCCCACGTGTCCGGCG |
| | | GCCTGAAGGAGGGCATCGAGACCCTGCTGATGACCCAGCGGCCCGC |
| | | CCTGTCCGGCGACGCCGTGCAGAAGATGCTGGGCCACTGCACCTTC |
| | | GAGCCCGCCGAGCCCAAGGCCGCCAAGAACACCTACACCGCCGAG |
| | | CGGTTCATCTGGCTGACCAAGCTGAACAACCTGCGGATCCTGGAGC |
| | | AGGGCTCCGAGCGGCCCCTGACCGACACCGAGCGGGCCACCCTGAT |
| | | GGACGAGCCCTACCGGAAGTCCAAGCTGACCTACGCCCAGGCCCGG |
| | | AAGCTGCTGGGCCTGGAGGACACCGCCTTCTTCAAGGGCCTGCGGT |
| | | ACGGCAAGGACAACGCCGAGGCCTCCACCCTGATGGAGATGAAGG |
| | | CCTACCACGCCATCTCCCGGGCCCTGGAGAAGGAGGGCCTGAAGGA |
| | | CAAGAAGTCCCCCCTGAACCTGTCCTCCGAGCTGCAGGACGAGATC |
| | | GGCACCGCCTTCTCCCTGTTCAAGACCGACGAGGACATCACCGGCC |
| | | GGCTGAAGGACCGGGTGCAGCCCGAGATCCTGGAGGCCCTGCTGA |
| | | AGCACATCTCCTTCGACAAGTTCGTGCAGATCTCCCTGAAGGCCCT |
| | | GCGGCGGATCGTGCCCCTGATGGAGCAGGGCAAGCGGTACGACGA |
| | | GGCCTGCGCCGAGATCTACGGCGACCACTACGGCAAGAAGAACAC |
| | | CGAGGAGAAGATCTACCTGCCCCCCATCCCCGCCGACGAGATCCGG |
| | | AACCCCGTGGTGCTGCGGGCCCTGTCCCAGGCCCGGAAGGTGATCA |
| | | ACGGCGTGGTGCGGCGGTACGGCTCCCCCGCCCGGATCCACATCGA |
| | | GACCGCCCGGGAGGTGGGCAAGTCCTTCAAGGACCGGAAGGAGAT |
| | | CGAGAAGCGGCAGGAGGAGAACCGGAAGGACCGGGAGAAGGCCG |
| | | CCGCCAAGTTCCGGGAGTACTTCCCCAACTTCGTGGGCGAGCCCAA |
| | | GTCCAAGGACATCCTGAAGCTGCGGCTGTACGAGCAGCAGCACGGC |
| | | AAGTGCCTGTACTCCGGCAAGGAGATCAACCTGGTGCGGCTGAACG |
| | | AGAAGGGCTACGTGGAGATCGACCACGCCCTGCCCTTCTCCCGGAC |
| | | CTGGGACGACTCCTTCAACAACAAGGTGCTGGTGCTGGGCTCCGAG |
| | | AACCAGAACAAGGGCAACCAGACCCCCTACGAGTACTTCAACGGC |
| | | AAGGACAACTCCCGGGAGTGGCAGGAGTTCAAGGCCCGGGTGGAG |
| | | ACCTCCCGGTTCCCCCGGTCCAAGAAGCAGCGGATCCTGCTGCAGA |
| | | AGTTCGACGAGGACGGCTTCAAGGAGTGCAACCTGAACGACACCC |
| | | GGTACGTGAACCGGTTCCTGTGCCAGTTCGTGGCCGACCACATCCT |
| | | GCTGACCGGCAAGGGCAAGCGGCGGGTGTTCGCCTCCAACGGCCA |
| | | GATCACCAACCTGCTGCGGGGCTTCTGGGGCCTGCGGAAGGTGCGG |
| | | GCCGAGAACGACCGGCACCACGCCCTGGACGCCGTGGTGGTGGCCT |
| | | GCTCCACCGTGGCCATGCAGCAGAAGATCACCCGGTTCGTGCGGTA |
| | | CAAGGAGATGAACGCCTTCGACGGCAAGACCATCGACAAGGAGAC |
| | | CGGCAAGGTGCTGCACCAGAAGACCCACTTCCCCCAGCCCTGGGAG |
| | | TTCTTCGCCCAGGAGGTGATGATCCGGGTGTTCGGCAAGCCCGACG |
| | | GCAAGCCCGAGTTCGAGGAGGCCGACACCCCCGAGAAGCTGCGGA |
| | | CCCTGCTGGCCGAGAAGCTGTCCTCCCGGCCCGAGGCCGTGCACGA |
| | | GTACGTGACCCCCCTGTTCGTGTCCCGGGCCCCCAACCGGAAGATG |
| | | TCCGGCGCCCACAAGGACACCCTGCGGTCCGCCAAGCGGTTCGTGA |
| | | AGCACAACGAGAAGATCTCCGTGAAGCGGGTGTGGCTGACCGAGA |
| | | TCAAGCTGGCCGACCTGGAGAACATGGTGAACTACAAGAACGGCC |
| | | GGGAGATCGAGCTGTACGAGGCCCTGAAGGCCCGGCTGGAGGCCT |
| | | ACGGCGGCAACGCCAAGCAGGCCTTCGACCCCAAGGACAACCCCTT |
| | | CTACAAGAAGGGCGGCCAGCTGGTGAAGGCCGTGCGGGTGGAGAA |
| | | GACCCAGGAGTCCGGCGTGCTGCTGAACAAGAAGAACGCCTACAC |
| | | CATCGCCGACAACGGCGACATGGTGCGGGTGGACGTGTTCTGCAAG |
| | | GTGGACAAGAAGGGCAAGAACCAGTACTTCATCGTGCCCATCTACG |
| | | CCTGGCAGGTGGCCGAGAACATCCTGCCCGACATCGACTGCAAGGG |
| | | CTACCGGATCGACGACTCCTACACCTTCTGCTTCTCCCTGCACAAGT |
| | | ACGACCTGATCGCCTTCCAGAAGGACGAGAAGTCCAAGGTGGAGTT |
| | | CGCCTACTACATCAACTGCGACTCCTCCAACGGCCGGTTCTACCTGG |
| | | CCTGGCACGACAAGGGCTCCAAGGAGCAGCAGTTCCGGATCTCCAC |
| | | CCAGAACCTGGTGCTGATCCAGAAGTACCAGGTGAACGAGCTGGGC |
| | | AAGGAGATCCGGCCCTGCCGGCTGAAGAAGCGGCCCCCCGTGCGG |
| | | UAA |
| 156 | Cas9 amino acid | MDKKYSIGLDIGTNSVGWAVITDEYKVPSKKFKVLGNTDRHSIKKNLI |
| | sequence | GALLFDSGETAEATRLKRTARRRYTRRKNRICYLQEIFSNEMAKVDDS |
| | | FFHRLEESFLVEEDKKHERHPIFGNIVDEVAYHEKYPTIYHLRKKLVDS |
| | | TDKADLRLIYLALAHMIKFRGHFLIEGDLNPDNSDVDKLFIQLVQTYN |
| | | QLFEENPINASGVDAKAILSARLSKSRRLENLIAQLPGEKKNGLFGNLIA |
| | | LSLGLTPNFKSNFDLAEDAKLQLSKDTYDDDLDNLLAQIGDQYADLFL |
| | | AAKNLSDAILLSDILRVNTEITKAPLSASMIKRYDEHHQDLTLLKALVR |
| | | QQLPEKYKEIFFDQSKNGYAGYIDGGASQEEFYKFIKPILEKMDGTEEL |
| | | LVKLNREDLLRKQRTFDNGSIPHQIHLGELHAILRRQEDFYPFLKDNRE |
| | | KIEKILTFRIPYYVGPLARGNSRFAWMTRKSEETITPWNFEEVVDKGAS |
| | | AQSFIERMTNFDKNLPNEKVLPKHSLLYEYFTVYNELTKVKYVTEGMR |
| | | KPAFLSGEQKKAIVDLLFKTNRKVTVKQLKEDYFKKIECFDSVEISGVE |
| | | DRFNASLGTYHDLLKIIKDKDFLDNEENEDILEDIVLTLTLFEDREMIEE |
| | | RLKTYAHLFDDKVMKQLKRRRYTGWGRLSRKLINGIRDKQSGKTILD |
| | | FLKSDGFANRNFMQLIHDDSLTFKEDIQKAQVSGQGDSLHEHIANLAG |
| | | SPAIKKGILQTVKVVDELVKVMGRHKPENIVIEMARENQTTQKGQKNS |
| | | RERMKRIEEGIKELGSQILKEHPVENTQLQNEKLYLYYLQNGRDMYVD |
| | | QELDINRLSDYDVDHIVPQSFLKDDSIDNKVLTRSDKNRGKSDNVPSEE |
| | | VVKKMKNYWRQLLNAKLITQRKFDNLTKAERGGLSELDKAGFIKRQL |
| | | VETRQITKHVAQILDSRMNTKYDENDKLIREVKVITLKSKLVSDFRKDF |
| | | QFYKVREINNYHHAHDAYLNAVVGTALIKKYPKLESEFVYGDYKVYD |
| | | VRKMIAKSEQEIGKATAKYFFYSNIMNFFKTEITLANGEIRKRPLIETNG |
| | | ETGEIVWDKGRDFATVRKVLSMPQVNIVKKTEVQTGGFSKESILPKRN |
| | | SDKLIARKKDWDPKKYGGFDSPTVAYSVLVVAKVEKGKSKKLKSVKE |
| | | LLGITIMERSSFEKNPIDFLEAKGYKEVKKDLIIKLPKYSLFELENGRKR |
| | | MLASAGELQKGNELALPSKYVNFLYLASHYEKLKGSPEDNEQKQLFV |
| | | EQHKHYLDEIIEQISEFSKRVILADANLDKVLSAYNKHRDKPIREQAENI |
| | | IHLFTLTNLGAPAAFKYFDTTIDRKRYTSTKEVLDATLIHQSITGLYETR |
| | | IDLSQLGGDGGGSPKKKRKV |
| 157 | Amino acid | MTGAAFKPNPINYILGLDIGIASVGWAMVEIDEEENPIRLIDLGVRVFER |
| | sequence for | AEVPKTGDSLAMARRLARSVRRLTRRRAHRLLRARRLLKREGVLQAA |
| | Nme2Cas9 | DFDENGLIKSLPNTPWQLRAAALDRKLTPLEWSAVLLHLIKHRGYLSQ |
| | encoded by | RKNEGETADKELGALLKGVANNAHALQTGDFRTPAELALNKFEKESG |
| | mRNA C | HIRNQRGDYSHTFSRKDLQAELILLFEKQKEFGNPHVSGGLKEGIETLL |
| | | MTQRPALSGDAVQKMLGHCTFEPAEPKAAKNTYTAERFIWLTKLNNL |
| | | RILEQGSERPLTDTERATLMDEPYRKSKLTYAQARKLLGLEDTAFFKG |
| | | LRYGKDNAEASTLMEMKAYHAISRALEKEGLKDKKSPLNLSSELQDEI |
| | | GTAFSLFKTDEDITGRLKDRVQPEILEALLKHISFDKFVQISLKALRRIVP |
| | | LMEQGKRYDEACAEIYGDHYGKKNTEEKIYLPPIPADEIRNPVVLRALS |
| | | QARKVINGVVRRYGSPARIHIETAREVGKSFKDRKEIEKRQEENRKDRE |
| | | KAAAKFREYFPNFVGEPKSKDILKLRLYEQQHGKCLYSGKEINLVRLN |
| | | EKGYVEIDHALPFSRTWDDSFNNKVLVLGSENQNKGNQTPYEYFNGK |
| | | DNSREWQEFKARVETSRFPRSKKQRILLQKFDEDGFKECNLNDTRYVN |
| | | RFLCQFVADHILLTGKGKRRVFASNGQITNLLRGFWGLRKVRAENDR |
| | | HHALDAVVVACSTVAMQQKITRFVRYKEMNAFDGKTIDKETGKVLH |
| | | QKTHFPQPWEFFAQEVMIRVFGKPDGKPEFEEADTPEKLRTLLAEKLSS |
| | | RPEAVHEYVTPLFVSRAPNRKMSGAHKDTLRSAKRFVKHNEKISVKR |
| | | VWLTEIKLADLENMVNYKNGREIELYEALKARLEAYGGNAKQAFDPK |
| | | DNPFYKKGGQLVKAVRVEKTQESGVLLNKKNAYTIADNGDMVRVDV |
| | | FCKVDKKGKNQYFIVPIYAWQVAENILPDIDCKGYRIDDSYTFCFSLHK |
| | | YDLIAFQKDEKSKVEFAYYINCDSSNGRFYLAWHDKGSKEQQFRISTQ |
| | | NLVLIQKYQVNELGKEIRPCRLKKRPPVRSGKRTADGSEFESPKKKRK |
| | | VE |
| 158 | Amino acid | MAAFKPNPINYILGLDIGIASVGWAMVEIDEEENPIRLIDLGVRVFERAE |
| | sequence for | VPKTGDSLAMARRLARSVRRLTRRRAHRLLRARRLLKREGVLQAADF |
| | Nme2Cas9 | DENGLIKSLPNTPWQLRAAALDRKLTPLEWSAVLLHLIKHRGYLSQRK |
| | encoded by | NEGETADKELGALLKGVANNAHALQTGDFRTPAELALNKFEKESGHI |
| | mRNA H | RNQRGDYSHTFSRKDLQAELILLFEKQKEFGNPHVSGGLKEGIETLLMT |
| | | QRPALSGDAVQKMLGHCTFEPAEPKAAKNTYTAERFIWLTKLNNLRIL |
| | | EQGSERPLTDTERATLMDEPYRKSKLTYAQARKLLGLEDTAFFKGLRY |
| | | GKDNAEASTLMEMKAYHAISRALEKEGLKDKKSPLNLSSELQDEIGTA |
| | | FSLFKTDEDITGRLKDRVQPEILEALLKHISFDKFVQISLKALRRIVPLME |
| | | QGKRYDEACAEIYGDHYGKKNTEEKIYLPPIPADEIRNPVVLRALSQAR |
| | | KVINGVVRRYGSPARIHIETAREVGKSFKDRKEIEKRQEENRKDREKA |
| | | AAKFREYFPNFVGEPKSKDILKLRLYEQQHGKCLYSGKEINLVRLNEK |
| | | GYVEIDHALPFSRTWDDSFNNKVLVLGSENQNKGNQTPYEYFNGKDN |
| | | SREWQEFKARVETSRFPRSKKQRILLQKFDEDGFKECNLNDTRYVNRF |
| | | LCQFVADHILLTGKGKRRVFASNGQITNLLRGFWGLRKVRAENDRHH |
| | | ALDAVVVACSTVAMQQKITRFVRYKEMNAFDGKTIDKETGKVLHQK |
| | | THFPQPWEFFAQEVMIRVFGKPDGKPEFEEADTPEKLRTLLAEKLSSRP |
| | | EAVHEYVTPLFVSRAPNRKMSGAHKDTLRSAKRFVKHNEKISVKRVW |
| | | LTEIKLADLENMVNYKNGREIELYEALKARLEAYGGNAKQAFDPKDN |
| | | PFYKKGGQLVKAVRVEKTQESGVLLNKKNAYTIADNGDMVRVDVFC |
| | | KVDKSGGGSPKKKRKVSGGSGKNQYFIVPIYAWQVAENILPDIDCKGY |
| | | RIDDSYTFCFSLHKYDLIAFQKDEKSKVEFAYYINCDSSNGRFYLAWH |
| | | DKGSKEQQFRISTQNLVLIQKYQVNELGKEIRPCRLKKRPPVR |
| 159 | Amino acid | MVPKKKRKVAAFKPNPINYILGLDIGIASVGWAMVEIDEEENPIRLIDL |
| | sequence for | GVRVFERAEVPKTGDSLAMARRLARSVRRLTRRRAHRLLRARRLLKR |
| | Nme2Cas9 | EGVLQAADFDENGLIKSLPNTPWQLRAAALDRKLTPLEWSAVLLHLIK |
| | encoded by | HRGYLSQRKNEGETADKELGALLKGVANNAHALQTGDFRTPAELALN |
| | mRNA I | KFEKESGHIRNQRGDYSHTFSRKDLQAELILLFEKQKEFGNPHVSGGLK |
| | | EGIETLLMTQRPALSGDAVQKMLGHCTFEPAEPKAAKNTYTAERFIWL |
| | | TKLNNLRILEQGSERPLTDTERATLMDEPYRKSKLTYAQARKLLGLED |
| | | TAFFKGLRYGKDNAEASTLMEMKAYHAISRALEKEGLKDKKSPLNLS |
| | | SELQDEIGTAFSLFKTDEDITGRLKDRVQPEILEALLKHISFDKFVQISLK |
| | | ALRRIVPLMEQGKRYDEACAEIYGDHYGKKNTEEKIYLPPIPADEIRNP |
| | | VVLRALSQARKVINGVVRRYGSPARIHIETAREVGKSFKDRKEIEKRQE |
| | | ENRKDREKAAAKFREYFPNFVGEPKSKDILKLRLYEQQHGKCLYSGKE |
| | | INLVRLNEKGYVEIDHALPFSRTWDDSFNNKVLVLGSENQNKGNQTPY |
| | | EYFNGKDNSREWQEFKARVETSRFPRSKKQRILLQKFDEDGFKECNLN |
| | | DTRYVNRFLCQFVADHILLTGKGKRRVFASNGQITNLLRGFWGLRKV |
| | | RAENDRHHALDAVVVACSTVAMQQKITRFVRYKEMNAFDGKTIDKE |
| | | TGKVLHQKTHFPQPWEFFAQEVMIRVFGKPDGKPEFEEADTPEKLRTL |
| | | LAEKLSSRPEAVHEYVTPLFVSRAPNRKMSGAHKDTLRSAKRFVKHNE |
| | | KISVKRVWLTEIKLADLENMVNYKNGREIELYEALKARLEAYGGNAK |
| | | QAFDPKDNPFYKKGGQLVKAVRVEKTQESGVLLNKKNAYTIADNGD |
| | | MVRVDVFCKVDKKGKNQYFIVPIYAWQVAENILPDIDCKGYRIDDSYT |
| | | FCFSLHKYDLIAFQKDEKSKVEFAYYINCDSSNGRFYLAWHDKGSKEQ |
| | | QFRISTQNLVLIQKYQVNELGKEIRPCRLKKRPPVRYPYDVPDYAAAPA |
| | | AKKKKLD |
| 160 | Amino acid | MAAFKPNPINYILGLDIGIASVGWAMVEIDEEENPIRLIDLGVRVFERAE |
| | sequence for | VPKTGDSLAMARRLARSVRRLTRRRAHRLLRARRLLKREGVLQAADF |
| | Nme2Cas9 | DENGLIKSLPNTPWQLRAAALDRKLTPLEWSAVLLHLIKHRGYLSQRK |
| | encoded by | NEGETADKELGALLKGVANNAHALQTGDFRTPAELALNKFEKESGHI |
| | mRNA J | RNQRGDYSHTFSRKDLQAELILLFEKQKEFGNPHVSGGLKEGIETLLMT |
| | | QRPALSGDAVQKMLGHCTFEPAEPKAAKNTYTAERFIWLTKLNNLRIL |
| | | EQGSERPLTDTERATLMDEPYRKSKLTYAQARKLLGLEDTAFFKGLRY |
| | | GKDNAEASTLMEMKAYHAISRALEKEGLKDKKSPLNLSSELQDEIGTA |
| | | FSLFKTDEDITGRLKDRVQPEILEALLKHISFDKFVQISLKALRRIVPLME |
| | | QGKRYDEACAEIYGDHYGKKNTEEKIYLPPIPADEIRNPVVLRALSQAR |
| | | KVINGVVRRYGSPARIHIETAREVGKSFKDRKEIEKRQEENRKDREKA |
| | | AAKFREYFPNFVGEPKSKDILKLRLYEQQHGKCLYSGKEINLVRLNEK |
| | | GYVEIDHALPFSRTWDDSFNNKVLVLGSENQNKGNQTPYEYFNGKDN |
| | | SREWQEFKARVETSRFPRSKKQRILLQKFDEDGFKECNLNDTRYVNRF |
| | | LCQFVADHILLTGKGKRRVFASNGQITNLLRGFWGLRKVRAENDRHH |
| | | ALDAVVVACSTVAMQQKITRFVRYKEMNAFDGKTIDKETGKVLHQK |
| | | THFPQPWEFFAQEVMIRVFGKPDGKPEFEEADTPEKLRTLLAEKLSSRP |
| | | EAVHEYVTPLFVSRAPNRKMSGAHKDTLRSAKRFVKHNEKISVKRVW |
| | | LTEIKLADLENMVNYKNGREIELYEALKARLEAYGGNAKQAFDPKDN |
| | | PFYKKGGQLVKAVRVEKTQESGVLLNKKNAYTIADNGDMVRVDVFC |
| | | KVDKKGKNQYFIVPIYAWQVAENILPDIDCKGYRIDDSYTFCFSLHKY |
| | | DLIAFQKDEKSKVEFAYYINCDSSNGRFYLAWHDKGSKEQQFRISTQN |
| | | LVLIQKYQVNELGKEIRPCRLKKRPPVR |
| 161 | Amino acid | MAAFKPNPINYILGLDIGIASVGWAMVEIDEEENPIRLIDLGVRVFERAE |
| | sequence for | VPKTGDSLAMARRLARSVRRLTRRRAHRLLRARRLLKREGVLQAADF |
| | Nme2Cas9 | DENGLIKSLPNTPWQLRAAALDRKLTPLEWSAVLLHLIKHRGYLSQRK |
| | encoded by | NEGETADKELGALLKGVANNAHALQTGDFRTPAELALNKFEKESGHI |
| | mRNA K | RNQRGDYSHTFSRKDLQAELILLFEKQKEFGNPHVSGGLKEGIETLLMT |
| | | QRPALSGDAVQKMLGHCTFEPAEPKAAKNTYTAERFIWLTKLNNLRIL |
| | | EQGSERPLTDTERATLMDEPYRKSKLTYAQARKLLGLEDTAFFKGLRY |
| | | GKDNAEASTLMEMKAYHAISRALEKEGLKDKKSPLNLSSELQDEIGTA |
| | | FSLFKTDEDITGRLKDRVQPEILEALLKHISFDKFVQISLKALRRIVPLME |
| | | QGKRYDEACAEIYGDHYGKKNTEEKIYLPPIPADEIRNPVVLRALSQAR |
| | | KVINGVVRRYGSPARIHIETAREVGKSFKDRKEIEKRQEENRKDREKA |
| | | AAKFREYFPNFVGEPKSKDILKLRLYEQQHGKCLYSGKEINLVRLNEK |
| | | GYVEIDHALPFSRTWDDSFNNKVLVLGSENQNKGNQTPYEYFNGKDN |
| | | SREWQEFKARVETSRFPRSKKQRILLQKFDEDGFKECNLNDTRYVNRF |
| | | LCQFVADHILLTGKGKRRVFASNGQITNLLRGFWGLRKVRAENDRHH |
| | | ALDAVVVACSTVAMQQKITRFVRYKEMNAFDGKTIDKETGKVLHQK |
| | | THFPQPWEFFAQEVMIRVFGKPDGKPEFEEADTPEKLRTLLAEKLSSRP |
| | | EAVHEYVTPLFVSRAPNRKMSGAHKDTLRSAKRFVKHNEKISVKRVW |
| | | LTEIKLADLENMVNYKNGREIELYEALKARLEAYGGNAKQAFDPKDN |
| | | PFYKKGGQLVKAVRVEKTQESGVLLNKKNAYTIADNGDMVRVDVFC |
| | | KVDKKGKNQYFIVPIYAWQVAENILPDIDCKGYRIDDSYTFCFSLHKY |
| | | DLIAFQKDEKSKVEFAYYINCDSSNGRFYLAWHDKGSKEQQFRISTQN |
| | | LVLIQKYQVNELGKEIRPCRLKKRPPVR |
| 162 | Amino acid | MDGSGGGSPKKKRKVGGSGGGAAFKPNPINYILGLDIGIASVGWAMV |
| | sequence for | EIDEEENPIRLIDLGVRVFERAEVPKTGDSLAMARRLARSVRRLTRRRA |
| | Nme2Cas9 | HRLLRARRLLKREGVLQAADFDENGLIKSLPNTPWQLRAAALDRKLTP |
| | encoded by | LEWSAVLLHLIKHRGYLSQRKNEGETADKELGALLKGVANNAHALQT |
| | mRNA L | GDFRTPAELALNKFEKESGHIRNQRGDYSHTFSRKDLQAELILLFEKQK |
| | | EFGNPHVSGGLKEGIETLLMTQRPALSGDAVQKMLGHCTFEPAEPKAA |
| | | KNTYTAERFIWLTKLNNLRILEQGSERPLTDTERATLMDEPYRKSKLTY |
| | | AQARKLLGLEDTAFFKGLRYGKDNAEASTLMEMKAYHAISRALEKEG |
| | | LKDKKSPLNLSSELQDEIGTAFSLFKTDEDITGRLKDRVQPEILEALLKH |
| | | ISFDKFVQISLKALRRIVPLMEQGKRYDEACAEIYGDHYGKKNTEEKIY |
| | | LPPIPADEIRNPVVLRALSQARKVINGVVRRYGSPARIHIETAREVGKSF |
| | | KDRKEIEKRQEENRKDREKAAAKFREYFPNFVGEPKSKDILKLRLYEQ |
| | | QHGKCLYSGKEINLVRLNEKGYVEIDHALPFSRTWDDSFNNKVLVLGS |
| | | ENQNKGNQTPYEYFNGKDNSREWQEFKARVETSRFPRSKKQRILLQKF |
| | | DEDGFKECNLNDTRYVNRFLCQFVADHILLTGKGKRRVFASNGQITNL |
| | | LRGFWGLRKVRAENDRHHALDAVVVACSTVAMQQKITRFVRYKEMN |
| | | AFDGKTIDKETGKVLHQKTHFPQPWEFFAQEVMIRVFGKPDGKPEFEE |
| | | ADTPEKLRTLLAEKLSSRPEAVHEYVTPLFVSRAPNRKMSGAHKDTLR |
| | | SAKRFVKHNEKISVKRVWLTEIKLADLENMVNYKNGREIELYEALKA |
| | | RLEAYGGNAKQAFDPKDNPFYKKGGQLVKAVRVEKTQESGVLLNKK |
| | | NAYTIADNGDMVRVDVFCKVDKKGKNQYFIVPIYAWQVAENILPDID |
| | | CKGYRIDDSYTFCFSLHKYDLIAFQKDEKSKVEFAYYINCDSSNGRFYL |
| | | AWHDKGSKEQQFRISTQNLVLIQKYQVNELGKEIRPCRLKKRPPVR |
| 163 | Amino acid | MDGSGGGSPKKKRKVGGSGGGAAFKPNPINYILGLDIGIASVGWAMV |
| | sequence for | EIDEEENPIRLIDLGVRVFERAEVPKTGDSLAMARRLARSVRRLTRRRA |
| | Nme2Cas9 with | HRLLRARRLLKREGVLQAADFDENGLIKSLPNTPWQLRAAALDRKLTP |
| | HiBiT tag | LEWSAVLLHLIKHRGYLSQRKNEGETADKELGALLKGVANNAHALQT |
| | encoded by | GDFRTPAELALNKFEKESGHIRNQRGDYSHTFSRKDLQAELILLFEKQK |
| | mRNA M | EFGNPHVSGGLKEGIETLLMTQRPALSGDAVQKMLGHCTFEPAEPKAA |
| | | KNTYTAERFIWLTKLNNLRILEQGSERPLTDTERATLMDEPYRKSKLTY |
| | | AQARKLLGLEDTAFFKGLRYGKDNAEASTLMEMKAYHAISRALEKEG |
| | | LKDKKSPLNLSSELQDEIGTAFSLFKTDEDITGRLKDRVQPEILEALLKH |
| | | ISFDKFVQISLKALRRIVPLMEQGKRYDEACAEIYGDHYGKKNTEEKIY |
| | | LPPIPADEIRNPVVLRALSQARKVINGVVRRYGSPARIHIETAREVGKSF |
| | | KDRKEIEKRQEENRKDREKAAAKFREYFPNFVGEPKSKDILKLRLYEQ |
| | | QHGKCLYSGKEINLVRLNEKGYVEIDHALPFSRTWDDSFNNKVLVLGS |
| | | ENQNKGNQTPYEYFNGKDNSREWQEFKARVETSRFPRSKKQRILLQKF |
| | | DEDGFKECNLNDTRYVNRFLCQFVADHILLTGKGKRRVFASNGQITNL |
| | | LRGFWGLRKVRAENDRHHALDAVVVACSTVAMQQKITRFVRYKEMN |
| | | AFDGKTIDKETGKVLHQKTHFPQPWEFFAQEVMIRVFGKPDGKPEFEE |
| | | ADTPEKLRTLLAEKLSSRPEAVHEYVTPLFVSRAPNRKMSGAHKDTLR |
| | | SAKRFVKHNEKISVKRVWLTEIKLADLENMVNYKNGREIELYEALKA |
| | | RLEAYGGNAKQAFDPKDNPFYKKGGQLVKAVRVEKTQESGVLLNKK |
| | | NAYTIADNGDMVRVDVFCKVDKKGKNQYFIVPIYAWQVAENILPDID |
| | | CKGYRIDDSYTFCFSLHKYDLIAFQKDEKSKVEFAYYINCDSSNGRFYL |
| | | AWHDKGSKEQQFRISTQNLVLIQKYQVNELGKEIRPCRLKKRPPVRSES |
| | | ATPESVSGWRLFKKIS |
| 164 | Amino acid | MDGSGGGSPKKKRKVGGSGGGAAFKPNPINYILGLDIGIASVGWAMV |
| | sequence for | EIDEEENPIRLIDLGVRVFERAEVPKTGDSLAMARRLARSVRRLTRRRA |
| | Nme2Cas9 | HRLLRARRLLKREGVLQAADFDENGLIKSLPNTPWQLRAAALDRKLTP |
| | encoded by | LEWSAVLLHLIKHRGYLSQRKNEGETADKELGALLKGVANNAHALQT |
| | mRNA N | GDFRTPAELALNKFEKESGHIRNQRGDYSHTFSRKDLQAELILLFEKQK |
| | | EFGNPHVSGGLKEGIETLLMTQRPALSGDAVQKMLGHCTFEPAEPKAA |
| | | KNTYTAERFIWLTKLNNLRILEQGSERPLTDTERATLMDEPYRKSKLTY |
| | | AQARKLLGLEDTAFFKGLRYGKDNAEASTLMEMKAYHAISRALEKEG |
| | | LKDKKSPLNLSSELQDEIGTAFSLFKTDEDITGRLKDRVQPEILEALLKH |
| | | ISFDKFVQISLKALRRIVPLMEQGKRYDEACAEIYGDHYGKKNTEEKIY |
| | | LPPIPADEIRNPVVLRALSQARKVINGVVRRYGSPARIHIETAREVGKSF |
| | | KDRKEIEKRQEENRKDREKAAAKFREYFPNFVGEPKSKDILKLRLYEQ |
| | | QHGKCLYSGKEINLVRLNEKGYVEIDHALPFSRTWDDSFNNKVLVLGS |
| | | ENQNKGNQTPYEYFNGKDNSREWQEFKARVETSRFPRSKKQRILLQKF |
| | | DEDGFKECNLNDTRYVNRFLCQFVADHILLTGKGKRRVFASNGQITNL |
| | | LRGFWGLRKVRAENDRHHALDAVVVACSTVAMQQKITRFVRYKEMN |
| | | AFDGKTIDKETGKVLHQKTHFPQPWEFFAQEVMIRVFGKPDGKPEFEE |
| | | ADTPEKLRTLLAEKLSSRPEAVHEYVTPLFVSRAPNRKMSGAHKDTLR |
| | | SAKRFVKHNEKISVKRVWLTEIKLADLENMVNYKNGREIELYEALKA |
| | | RLEAYGGNAKQAFDPKDNPFYKKGGQLVKAVRVEKTQESGVLLNKK |
| | | NAYTIADNGDMVRVDVFCKVDKKGKNQYFIVPIYAWQVAENILPDID |
| | | CKGYRIDDSYTFCFSLHKYDLIAFQKDEKSKVEFAYYINCDSSNGRFYL |
| | | AWHDKGSKEQQFRISTQNLVLIQKYQVNELGKEIRPCRLKKRPPVRSG |
| | | KRTADGSGGGSPAAKKKKLD |
| 165 | Amino acid | MDGSGGGSPKKKRKVEDKRPAATKKAGQAKKKKGGSGGGAAFKPNP |
| | sequence for | INYILGLDIGIASVGWAMVEIDEEENPIRLIDLGVRVFERAEVPKTGDSL |
| | Nme2Cas9 | AMARRLARSVRRLTRRRAHRLLRARRLLKREGVLQAADFDENGLIKS |
| | encoded by | LPNTPWQLRAAALDRKLTPLEWSAVLLHLIKHRGYLSQRKNEGETAD |
| | mRNA O | KELGALLKGVANNAHALQTGDFRTPAELALNKFEKESGHIRNQRGDY |
| | | SHTFSRKDLQAELILLFEKQKEFGNPHVSGGLKEGIETLLMTQRPALSG |
| | | DAVQKMLGHCTFEPAEPKAAKNTYTAERFIWLTKLNNLRILEQGSERP |
| | | LTDTERATLMDEPYRKSKLTYAQARKLLGLEDTAFFKGLRYGKDNAE |
| | | ASTLMEMKAYHAISRALEKEGLKDKKSPLNLSSELQDEIGTAFSLFKTD |
| | | EDITGRLKDRVQPEILEALLKHISFDKFVQISLKALRRIVPLMEQGKRYD |
| | | EACAEIYGDHYGKKNTEEKIYLPPIPADEIRNPVVLRALSQARKVINGV |
| | | VRRYGSPARIHIETAREVGKSFKDRKEIEKRQEENRKDREKAAAKFRE |
| | | YFPNFVGEPKSKDILKLRLYEQQHGKCLYSGKEINLVRLNEKGYVEID |
| | | HALPFSRTWDDSFNNKVLVLGSENQNKGNQTPYEYFNGKDNSREWQE |
| | | FKARVETSRFPRSKKQRILLQKFDEDGFKECNLNDTRYVNRFLCQFVA |
| | | DHILLTGKGKRRVFASNGQITNLLRGFWGLRKVRAENDRHHALDAVV |
| | | VACSTVAMQQKITRFVRYKEMNAFDGKTIDKETGKVLHQKTHFPQPW |
| | | EFFAQEVMIRVFGKPDGKPEFEEADTPEKLRTLLAEKLSSRPEAVHEYV |
| | | TPLFVSRAPNRKMSGAHKDTLRSAKRFVKHNEKISVKRVWLTEIKLAD |
| | | LENMVNYKNGREIELYEALKARLEAYGGNAKQAFDPKDNPFYKKGG |
| | | QLVKAVRVEKTQESGVLLNKKNAYTIADNGDMVRVDVFCKVDKKGK |
| | | NQYFIVPIYAWQVAENILPDIDCKGYRIDDSYTFCFSLHKYDLIAFQKD |
| | | EKSKVEFAYYINCDSSNGRFYLAWHDKGSKEQQFRISTQNLVLIQKYQ |
| | | VNELGKEIRPCRLKKRPPVR |
| 166 | Amino acid | MDGSGGGSPKKKRKVEDKRPAATKKAGQAKKKKGGSGGGAAFKPNP |
| | sequence for | INYILGLDIGIASVGWAMVEIDEEENPIRLIDLGVRVFERAEVPKTGDSL |
| | Nme2Cas9 with | AMARRLARSVRRLTRRRAHRLLRARRLLKREGVLQAADFDENGLIKS |
| | HiBiT tag | LPNTPWQLRAAALDRKLTPLEWSAVLLHLIKHRGYLSQRKNEGETAD |
| | encoded by | KELGALLKGVANNAHALQTGDFRTPAELALNKFEKESGHIRNQRGDY |
| | mRNA P | SHTFSRKDLQAELILLFEKQKEFGNPHVSGGLKEGIETLLMTQRPALSG |
| | | DAVQKMLGHCTFEPAEPKAAKNTYTAERFIWLTKLNNLRILEQGSERP |
| | | LTDTERATLMDEPYRKSKLTYAQARKLLGLEDTAFFKGLRYGKDNAE |
| | | ASTLMEMKAYHAISRALEKEGLKDKKSPLNLSSELQDEIGTAFSLFKTD |
| | | EDITGRLKDRVQPEILEALLKHISFDKFVQISLKALRRIVPLMEQGKRYD |
| | | EACAEIYGDHYGKKNTEEKIYLPPIPADEIRNPVVLRALSQARKVINGV |
| | | VRRYGSPARIHIETAREVGKSFKDRKEIEKRQEENRKDREKAAAKFRE |
| | | YFPNFVGEPKSKDILKLRLYEQQHGKCLYSGKEINLVRLNEKGYVEID |
| | | HALPFSRTWDDSFNNKVLVLGSENQNKGNQTPYEYFNGKDNSREWQE |
| | | FKARVETSRFPRSKKQRILLQKFDEDGFKECNLNDTRYVNRFLCQFVA |
| | | DHILLTGKGKRRVFASNGQITNLLRGFWGLRKVRAENDRHHALDAVV |
| | | VACSTVAMQQKITRFVRYKEMNAFDGKTIDKETGKVLHQKTHFPQPW |
| | | EFFAQEVMIRVFGKPDGKPEFEEADTPEKLRTLLAEKLSSRPEAVHEYV |
| | | TPLFVSRAPNRKMSGAHKDTLRSAKRFVKHNEKISVKRVWLTEIKLAD |
| | | LENMVNYKNGREIELYEALKARLEAYGGNAKQAFDPKDNPFYKKGG |
| | | QLVKAVRVEKTQESGVLLNKKNAYTIADNGDMVRVDVFCKVDKKGK |
| | | NQYFIVPIYAWQVAENILPDIDCKGYRIDDSYTFCFSLHKYDLIAFQKD |
| | | EKSKVEFAYYINCDSSNGRFYLAWHDKGSKEQQFRISTQNLVLIQKYQ |
| | | VNELGKEIRPCRLKKRPPVRSESATPESVSGWRLFKKIS |
| 167 | Amino acid | MDGSGGGSEDKRPAATKKAGQAKKKKGGSGGGAAFKPNPINYILGLD |
| | sequence for | IGIASVGWAMVEIDEEENPIRLIDLGVRVFERAEVPKTGDSLAMARRLA |
| | Nme2Cas9 | RSVRRLTRRRAHRLLRARRLLKREGVLQAADFDENGLIKSLPNTPWQL |
| | encoded by | RAAALDRKLTPLEWSAVLLHLIKHRGYLSQRKNEGETADKELGALLK |
| | mRNA Q | GVANNAHALQTGDFRTPAELALNKFEKESGHIRNQRGDYSHTFSRKDL |
| | | QAELILLFEKQKEFGNPHVSGGLKEGIETLLMTQRPALSGDAVQKMLG |
| | | HCTFEPAEPKAAKNTYTAERFIWLTKLNNLRILEQGSERPLTDTERATL |
| | | MDEPYRKSKLTYAQARKLLGLEDTAFFKGLRYGKDNAEASTLMEMK |
| | | AYHAISRALEKEGLKDKKSPLNLSSELQDEIGTAFSLFKTDEDITGRLK |
| | | DRVQPEILEALLKHISFDKFVQISLKALRRIVPLMEQGKRYDEACAEIY |
| | | GDHYGKKNTEEKIYLPPIPADEIRNPVVLRALSQARKVINGVVRRYGSP |
| | | ARIHIETAREVGKSFKDRKEIEKRQEENRKDREKAAAKFREYFPNFVGE |
| | | PKSKDILKLRLYEQQHGKCLYSGKEINLVRLNEKGYVEIDHALPFSRT |
| | | WDDSFNNKVLVLGSENQNKGNQTPYEYFNGKDNSREWQEFKARVET |
| | | SRFPRSKKQRILLQKFDEDGFKECNLNDTRYVNRFLCQFVADHILLTGK |
| | | GKRRVFASNGQITNLLRGFWGLRKVRAENDRHHALDAVVVACSTVA |
| | | MQQKITRFVRYKEMNAFDGKTIDKETGKVLHQKTHFPQPWEFFAQEV |
| | | MIRVFGKPDGKPEFEEADTPEKLRTLLAEKLSSRPEAVHEYVTPLFVSR |
| | | APNRKMSGAHKDTLRSAKRFVKHNEKISVKRVWLTEIKLADLENMVN |
| | | YKNGREIELYEALKARLEAYGGNAKQAFDPKDNPFYKKGGQLVKAV |
| | | RVEKTQESGVLLNKKNAYTIADNGDMVRVDVFCKVDKKGKNQYFIV |
| | | PIYAWQVAENILPDIDCKGYRIDDSYTFCFSLHKYDLIAFQKDEKSKVE |
| | | FAYYINCDSSNGRFYLAWHDKGSKEQQFRISTQNLVLIQKYQVNELGK |
| | | EIRPCRLKKRPPVR |
| 168 | mRNA C | GGGAAGCUCAGAAUAAACGCUCAACUUUGGCCGGAUCUGCCACCA |
| | encoding | UGACCGGUGCCGCCUUCAAGCCCAACCCCAUCAACUACAUCCUGG |
| | Nme2Cas9 | GCCUGGACAUCGGCAUCGCCUCCGUGGGCUGGGCCAUGGUGGAGA |
| | | UCGACGAGGAGGAGAACCCCAUCCGGCUGAUCGACCUGGGCGUGC |
| | | GGGUGUUCGAGCGGGCCGAGGUGCCCAAGACCGGCGACUCCCUGG |
| | | CCAUGGCCCGGCGGCUGGCCCGGUCCGUGCGGCGGCUGACCCGGC |
| | | GGCGGGCCCACCGGCUGCUGCGGGCCCGGCGGCUGCUGAAGCGGG |
| | | AGGGCGUGCUGCAGGCCGCCGACUUCGACGAGAACGGCCUGAUCA |
| | | AGUCCCUGCCCAACACCCCCUGGCAGCUGCGGGCCGCCGCCCUGG |
| | | ACCGGAAGCUGACCCCCCUGGAGUGGUCCGCCGUGCUGCUGCACC |
| | | UGAUCAAGCACCGGGGCUACCUGUCCCAGCGGAAGAACGAGGGCG |
| | | AGACCGCCGACAAGGAGCUGGGCGCCCUGCUGAAGGGCGUGGCCA |
| | | ACAACGCCCACGCCCUGCAGACCGGCGACUUCCGGACCCCCGCCG |
| | | AGCUGGCCCUGAACAAGUUCGAGAAGGAGUCCGGCCACAUCCGGA |
| | | ACCAGCGGGGCGACUACUCCCACACCUUCUCCCGGAAGGACCUGC |
| | | AGGCCGAGCUGAUCCUGCUGUUCGAGAAGCAGAAGGAGUUCGGC |
| | | AACCCCCACGUGUCCGGCGGCCUGAAGGAGGGCAUCGAGACCCUG |
| | | CUGAUGACCCAGCGGCCCGCCCUGUCCGGCGACGCCGUGCAGAAG |
| | | AUGCUGGGCCACUGCACCUUCGAGCCCGCCGAGCCCAAGGCCGCC |
| | | AAGAACACCUACACCGCCGAGCGGUUCAUCUGGCUGACCAAGCUG |
| | | AACAACCUGCGGAUCCUGGAGCAGGGCUCCGAGCGGCCCCUGACC |
| | | GACACCGAGCGGGCCACCCUGAUGGACGAGCCCUACCGGAAGUCC |
| | | AAGCUGACCUACGCCCAGGCCCGGAAGCUGCUGGGCCUGGAGGAC |
| | | ACCGCCUUCUUCAAGGGCCUGCGGUACGGCAAGGACAACGCCGAG |
| | | GCCUCCACCCUGAUGGAGAUGAAGGCCUACCACGCCAUCUCCCGG |
| | | GCCCUGGAGAAGGAGGGCCUGAAGGACAAGAAGUCCCCCCUGAAC |
| | | CUGUCCUCCGAGCUGCAGGACGAGAUCGGCACCGCCUUCUCCCUG |
| | | UUCAAGACCGACGAGGACAUCACCGGCCGGCUGAAGGACCGGGUG |
| | | CAGCCCGAGAUCCUGGAGGCCCUGCUGAAGCACAUCUCCUUCGAC |
| | | AAGUUCGUGCAGAUCUCCCUGAAGGCCCUGCGGCGGAUCGUGCCC |
| | | CUGAUGGAGCAGGGCAAGCGGUACGACGAGGCCUGCGCCGAGAU |
| | | CUACGGCGACCACUACGGCAAGAAGAACACCGAGGAGAAGAUCU |
| | | ACCUGCCCCCCAUCCCCGCCGACGAGAUCCGGAACCCCGUGGUGC |
| | | UGCGGGCCCUGUCCCAGGCCCGGAAGGUGAUCAACGGCGUGGUGC |
| | | GGCGGUACGGCUCCCCCGCCCGGAUCCACAUCGAGACCGCCCGGG |
| | | AGGUGGGCAAGUCCUUCAAGGACCGGAAGGAGAUCGAGAAGCGG |
| | | CAGGAGGAGAACCGGAAGGACCGGGAGAAGGCCGCCGCCAAGUU |
| | | CCGGGAGUACUUCCCCAACUUCGUGGGCGAGCCCAAGUCCAAGGA |
| | | CAUCCUGAAGCUGCGGCUGUACGAGCAGCAGCACGGCAAGUGCCU |
| | | GUACUCCGGCAAGGAGAUCAACCUGGUGCGGCUGAACGAGAAGG |
| | | GCUACGUGGAGAUCGACCACGCCCUGCCCUUCUCCCGGACCUGGG |
| | | ACGACUCCUUCAACAACAAGGUGCUGGUGCUGGGCUCCGAGAACC |
| | | AGAACAAGGGCAACCAGACCCCCUACGAGUACUUCAACGGCAAGG |
| | | ACAACUCCCGGGAGUGGCAGGAGUUCAAGGCCCGGGUGGAGACC |
| | | UCCCGGUUCCCCCGGUCCAAGAAGCAGCGGAUCCUGCUGCAGAAG |
| | | UUCGACGAGGACGGCUUCAAGGAGUGCAACCUGAACGACACCCGG |
| | | UACGUGAACCGCUUCCUGUGCCAGUUCGUGGCCGACCACAUCCUG |
| | | CUGACCGGCAAGGGCAAGCGGCGGGUGUUCGCCUCCAACGGCCAG |
| | | AUCACCAACCUGCUGCGGGGCUUCUGGGGCCUGCGGAAGGUGCGG |
| | | GCCGAGAACGACCGGCACCACGCCCUGGACGCCGUGGUGGUGGCC |
| | | UGCUCCACCGUGGCCAUGCAGCAGAAGAUCACCCGGUUCGUGCGG |
| | | UACAAGGAGAUGAACGCCUUCGACGGCAAGACCAUCGACAAGGA |
| | | GACCGGCAAGGUGCUGCACCAGAAGACCCACUUCCCCCAGCCCUG |
| | | GGAGUUCUUCGCCCAGGAGGUGAUGAUCCGGGUGUUCGGCAAGC |
| | | CCGACGGCAAGCCCGAGUUCGAGGAGGCCGACACCCCCGAGAAGC |
| | | UGCGGACCCUGCUGGCCGAGAAGCUGUCCUCCCGGCCCGAGGCCG |
| | | UGCACGAGUACGUGACCCCCCUGUUCGUGUCCCGGGCCCCCAACC |
| | | GGAAGAUGUCCGGCGCCCACAAGGACACCCUGCGGUCCGCCAAGC |
| | | GGUUCGUGAAGCACAACGAGAAGAUCUCCGUGAAGCGGGUGUGG |
| | | CUGACCGAGAUCAAGCUGGCCGACCUGGAGAACAUGGUGAACUA |
| | | CAAGAACGGCCGGGAGAUCGAGCUGUACGAGGCCCUGAAGGCCCG |
| | | GCUGGAGGCCUACGGCGGCAACGCCAAGCAGGCCUUCGACCCCAA |
| | | GGACAACCCCUUCUACAAGAAGGGCGGCCAGCUGGUGAAGGCCGU |
| | | GCGGGUGGAGAAGACCCAGGAGUCCGGCGUGCUGCUGAACAAGA |
| | | AGAACGCCUACACCAUCGCCGACAACGGCGACAUGGUGCGGGUGG |
| | | ACGUGUUCUGCAAGGUGGACAAGAAGGGCAAGAACCAGUACUUC |
| | | AUCGUGCCCAUCUACGCCUGGCAGGUGGCCGAGAACAUCCUGCCC |
| | | GACAUCGACUGCAAGGGCUACCGGAUCGACGACUCCUACACCUUC |
| | | UGCUUCUCCCUGCACAAGUACGACCUGAUCGCCUUCCAGAAGGAC |
| | | GAGAAGUCCAAGGUGGAGUUCGCCUACUACAUCAACUGCGACUCC |
| | | UCCAACGGCCGGUUCUACCUGGCCUGGCACGACAAGGGCUCCAAG |
| | | GAGCAGCAGUUCCGGAUCUCCACCCAGAACCUGGUGCUGAUCCAG |
| | | AAGUACCAGGUGAACGAGCUGGGCAAGGAGAUCCGGCCCUGCCG |
| | | GCUGAAGAAGCGGCCCCCCGUGCGGUCCGGAAAGCGGACCGCCGA |
| | | CGGCUCCGAGUUCGAGUCCCCCAAGAAGAAGCGGAAGGUGGAGU |
| | | AGCUAGCACCAGCCUCAAGAACACCCGAAUGGAGUCUCUAAGCUA |
| | | CAUAAUACCAACUUACACUUUACAAAAUGUUGUCCCCCAAAAUG |
| | | UAGCCAUUCGUAUCUGCUCCUAAUAAAAAGAAAGUUUCUUCACA |
| | | UUCUCUCGAGAAAAAAAAAAAAUGGAAAAAAAAAAAACGGAAAA |
| | | AAAAAAAAGGUAAAAAAAAAAAAUAUAAAAAAAAAAAACAUAAA |
| | | AAAAAAAAACGAAAAAAAAAAAACGUAAAAAAAAAAAACUCAAA |
| | | AAAAAAAAAGAUAAAAAAAAAAAACCUAAAAAAAAAAAAUGUAA |
| | | AAAAAAAAAAGGGAAAAAAAAAAAACGCAAAAAAAAAAAACACA |
| | | AAAAAAAAAAAUGCAAAAAAAAAAAAUCGAAAAAAAAAAAAUCU |
| | | AAAAAAAAAAAACGAAAAAAAAAAAACCCAAAAAAAAAAAAGAC |
| | | AAAAAAAAAAAAUAGAAAAAAAAAAAAGUUAAAAAA |
| 169 | mRNA H | GGGaagctcagaataaacgctcaactttggccggatctgccacCATGGCCGCCTTCAAGCC |
| | encoding | CAACCCCATCAACTACATCCTGGGCCTGGACATCGGCATCGCCTCC |
| | Nme2Cas9 | GTGGGCTGGGCCATGGTGGAGATCGACGAGGAGGAGAACCCCATC |
| | | CGGCTGATCGACCTGGGCGTGCGGGTGTTCGAGCGGGCCGAGGTGC |
| | | CCAAGACCGGCGACTCCCTGGCCATGGCCCGGCGGCTGGCCCGGTC |
| | | CGTGCGGCGGCTGACCCGGCGGCGGGCCCACCGGCTGCTGCGGGCC |
| | | CGGCGGCTGCTGAAGCGGGAGGGCGTGCTGCAGGCCGCCGACTTCG |
| | | ACGAGAACGGCCTGATCAAGTCCCTGCCCAACACCCCCTGGCAGCT |
| | | GCGGGCCGCCGCCCTGGACCGGAAGCTGACCCCCCTGGAGTGGTCC |
| | | GCCGTGCTGCTGCACCTGATCAAGCACCGGGGCTACCTGTCCCAGC |
| | | GGAAGAACGAGGGCGAGACCGCCGACAAGGAGCTGGGCGCCCTGC |
| | | TGAAGGGCGTGGCCAACAACGCCCACGCCCTGCAGACCGGCGACTT |
| | | CCGGACCCCCGCCGAGCTGGCCCTGAACAAGTTCGAGAAGGAGTCC |
| | | GGCCACATCCGGAACCAGCGGGGCGACTACTCCCACACCTTCTCCC |
| | | GGAAGGACCTGCAGGCCGAGCTGATCCTGCTGTTCGAGAAGCAGA |
| | | AGGAGTTCGGCAACCCCCACGTGTCCGGCGGCCTGAAGGAGGGCAT |
| | | CGAGACCCTGCTGATGACCCAGCGGCCCGCCCTGTCCGGCGACGCC |
| | | GTGCAGAAGATGCTGGGCCACTGCACCTTCGAGCCCGCCGAGCCCA |
| | | AGGCCGCCAAGAACACCTACACCGCCGAGCGGTTCATCTGGCTGAC |
| | | CAAGCTGAACAACCTGCGGATCCTGGAGCAGGGCTCCGAGCGGCCC |
| | | CTGACCGACACCGAGCGGGCCACCCTGATGGACGAGCCCTACCGGA |
| | | AGTCCAAGCTGACCTACGCCCAGGCCCGGAAGCTGCTGGGCCTGGA |
| | | GGACACCGCCTTCTTCAAGGGCCTGCGGTACGGCAAGGACAACGCC |
| | | GAGGCCTCCACCCTGATGGAGATGAAGGCCTACCACGCCATCTCCC |
| | | GGGCCCTGGAGAAGGAGGGCCTGAAGGACAAGAAGTCCCCCCTGA |
| | | ACCTGTCCTCCGAGCTGCAGGACGAGATCGGCACCGCCTTCTCCCT |
| | | GTTCAAGACCGACGAGGACATCACCGGCCGGCTGAAGGACCGGGT |
| | | GCAGCCCGAGATCCTGGAGGCCCTGCTGAAGCACATCTCCTTCGAC |
| | | AAGTTCGTGCAGATCTCCCTGAAGGCCCTGCGGCGGATCGTGCCCC |
| | | TGATGGAGCAGGGCAAGCGGTACGACGAGGCCTGCGCCGAGATCT |
| | | ACGGCGACCACTACGGCAAGAAGAACACCGAGGAGAAGATCTACC |
| | | TGCCCCCCATCCCCGCCGACGAGATCCGGAACCCCGTGGTGCTGCG |
| | | GGCCCTGTCCCAGGCCCGGAAGGTGATCAACGGCGTGGTGCGGCGG |
| | | TACGGCTCCCCCGCCCGGATCCACATCGAGACCGCCCGGGAGGTGG |
| | | GCAAGTCCTTCAAGGACCGGAAGGAGATCGAGAAGCGGCAGGAGG |
| | | AGAACCGGAAGGACCGGGAGAAGGCCGCCGCCAAGTTCCGGGAGT |
| | | ACTTCCCCAACTTCGTGGGCGAGCCCAAGTCCAAGGACATCCTGAA |
| | | GCTGCGGCTGTACGAGCAGCAGCACGGCAAGTGCCTGTACTCCGGC |
| | | AAGGAGATCAACCTGGTGCGGCTGAACGAGAAGGGCTACGTGGAG |
| | | ATCGACCACGCCCTGCCCTTCTCCCGGACCTGGGACGACTCCTTCAA |
| | | CAACAAGGTGCTGGTGCTGGGCTCCGAGAACCAGAACAAGGGCAA |
| | | CCAGACCCCCTACGAGTACTTCAACGGCAAGGACAACTCCCGGGAG |
| | | TGGCAGGAGTTCAAGGCCCGGGTGGAGACCTCCCGGTTCCCCCGGT |
| | | CCAAGAAGCAGCGGATCCTGCTGCAGAAGTTCGACGAGGACGGCTT |
| | | CAAGGAGTGCAACCTGAACGACACCCGGTACGTGAACCGGTTCCTG |
| | | TGCCAGTTCGTGGCCGACCACATCCTGCTGACCGGCAAGGGCAAGC |
| | | GGCGGGTGTTCGCCTCCAACGGCCAGATCACCAACCTGCTGCGGGG |
| | | CTTCTGGGGCCTGCGGAAGGTGCGGGCCGAGAACGACCGGCACCA |
| | | CGCCCTGGACGCCGTGGTGGTGGCCTGCTCCACCGTGGCCATGCAG |
| | | CAGAAGATCACCCGGTTCGTGCGGTACAAGGAGATGAACGCCTTCG |
| | | ACGGCAAGACCATCGACAAGGAGACCGGCAAGGTGCTGCACCAGA |
| | | AGACCCACTTCCCCCAGCCCTGGGAGTTCTTCGCCCAGGAGGTGAT |
| | | GATCCGGGTGTTCGGCAAGCCCGACGGCAAGCCCGAGTTCGAGGA |
| | | GGCCGACACCCCCGAGAAGCTGCGGACCCTGCTGGCCGAGAAGCT |
| | | GTCCTCCCGGCCCGAGGCCGTGCACGAGTACGTGACCCCCCTGTTC |
| | | GTGTCCCGGGCCCCCAACCGGAAGATGTCCGGCGCCCACAAGGACA |
| | | CCCTGCGGTCCGCCAAGCGGTTCGTGAAGCACAACGAGAAGATCTC |
| | | CGTGAAGCGGGTGTGGCTGACCGAGATCAAGCTGGCCGACCTGGA |
| | | GAACATGGTGAACTACAAGAACGGCCGGGAGATCGAGCTGTACGA |
| | | GGCCCTGAAGGCCCGGCTGGAGGCCTACGGCGGCAACGCCAAGCA |
| | | GGCCTTCGACCCCAAGGACAACCCCTTCTACAAGAAGGGCGGCCAG |
| | | CTGGTGAAGGCCGTGCGGGTGGAGAAGACCCAGGAGTCCGGCGTG |
| | | CTGCTGAACAAGAAGAACGCCTACACCATCGCCGACAACGGCGAC |
| | | ATGGTGCGGGTGGACGTGTTCTGCAAGGTGGACAAGTCCGGCGGCG |
| | | GCTCCCCCAAGAAGAAGCGGAAGGTGTCCGGCGGCTCCGGCAAGA |
| | | ACCAGTACTTCATCGTGCCCATCTACGCCTGGCAGGTGGCCGAGAA |
| | | CATCCTGCCCGACATCGACTGCAAGGGCTACCGGATCGACGACTCC |
| | | TACACCTTCTGCTTCTCCCTGCACAAGTACGACCTGATCGCCTTCCA |
| | | GAAGGACGAGAAGTCCAAGGTGGAGTTCGCCTACTACATCAACTGC |
| | | GACTCCTCCAACGGCCGGTTCTACCTGGCCTGGCACGACAAGGGCT |
| | | CCAAGGAGCAGCAGTTCCGGATCTCCACCCAGAACCTGGTGCTGAT |
| | | CCAGAAGTACCAGGTGAACGAGCTGGGCAAGGAGATCCGGCCCTG |
| | | CCGGCTGAAGAAGCGGCCCCCCGTGCGGTAGCTAGCaccagcctcaagaac |
| | | acccgaatggagtctctaagctacataataccaacttacactttacaaaatgttgtcccccaaaatgtagccattcgt |
| | | atctgctcctaataaaaagaaagtttcttcacattctCTCGAGAAAAAAAAAAAATGGAAA |
| | | AAAAAAAAACGGAAAAAAAAAAAAGGTAAAAAAAAAAAATATAA |
| | | AAAAAAAAAACATAAAAAAAAAAAACGAAAAAAAAAAAACGTAA |
| | | AAAAAAAAAACTCAAAAAAAAAAAAGATAAAAAAAAAAAACCTA |
| | | AAAAAAAAAAATGTAAAAAAAAAAAAGGGAAAAAAAAAAAACGC |
| | | AAAAAAAAAAAACACAAAAAAAAAAAATGCAAAAAAAAAAAATC |
| | | GAAAAAAAAAAAATCTAAAAAAAAAAAACGAAAAAAAAAAAACC |
| | | CAAAAAAAAAAAAGACAAAAAAAAAAAATAGAAAAAAAAAAAAG |
| | | TTAAAAAAAAAAAACTGAAAAAAAAAAAATTTAAAAAAAAAAAAT |
| 170 | mRNA I | GGGaagctcagaataaacgctcaactttggccggatctgccacCATGGTGCCCAAGAAGAA |
| | encoding | GCGGAAGGTGGCCGCCTTCAAGCCCAACCCCATCAACTACATCCTG |
| | Nme2Cas9 | GGCCTGGACATCGGCATCGCCTCCGTGGGCTGGGCCATGGTGGAGA |
| | | TCGACGAGGAGGAGAACCCCATCCGGCTGATCGACCTGGGCGTGCG |
| | | GGTGTTCGAGCGGGCCGAGGTGCCCAAGACCGGCGACTCCCTGGCC |
| | | ATGGCCCGGCGGCTGGCCCGGTCCGTGCGGCGGCTGACCCGGCGGC |
| | | GGGCCCACCGGCTGCTGCGGGCCCGGCGGCTGCTGAAGCGGGAGG |
| | | GCGTGCTGCAGGCCGCCGACTTCGACGAGAACGGCCTGATCAAGTC |
| | | CCTGCCCAACACCCCCTGGCAGCTGCGGGCCGCCGCCCTGGACCGG |
| | | AAGCTGACCCCCCTGGAGTGGTCCGCCGTGCTGCTGCACCTGATCA |
| | | AGCACCGGGGCTACCTGTCCCAGCGGAAGAACGAGGGCGAGACCG |
| | | CCGACAAGGAGCTGGGCGCCCTGCTGAAGGGCGTGGCCAACAACG |
| | | CCCACGCCCTGCAGACCGGCGACTTCCGGACCCCCGCCGAGCTGGC |
| | | CCTGAACAAGTTCGAGAAGGAGTCCGGCCACATCCGGAACCAGCG |
| | | GGGCGACTACTCCCACACCTTCTCCCGGAAGGACCTGCAGGCCGAG |
| | | CTGATCCTGCTGTTCGAGAAGCAGAAGGAGTTCGGCAACCCCCACG |
| | | TGTCCGGCGGCCTGAAGGAGGGCATCGAGACCCTGCTGATGACCCA |
| | | GCGGCCCGCCCTGTCCGGCGACGCCGTGCAGAAGATGCTGGGCCAC |
| | | TGCACCTTCGAGCCCGCCGAGCCCAAGGCCGCCAAGAACACCTACA |
| | | CCGCCGAGCGGTTCATCTGGCTGACCAAGCTGAACAACCTGCGGAT |
| | | CCTGGAGCAGGGCTCCGAGCGGCCCCTGACCGACACCGAGCGGGC |
| | | CACCCTGATGGACGAGCCCTACCGGAAGTCCAAGCTGACCTACGCC |
| | | CAGGCCCGGAAGCTGCTGGGCCTGGAGGACACCGCCTTCTTCAAGG |
| | | GCCTGCGGTACGGCAAGGACAACGCCGAGGCCTCCACCCTGATGGA |
| | | GATGAAGGCCTACCACGCCATCTCCCGGGCCCTGGAGAAGGAGGG |
| | | CCTGAAGGACAAGAAGTCCCCCCTGAACCTGTCCTCCGAGCTGCAG |
| | | GACGAGATCGGCACCGCCTTCTCCCTGTTCAAGACCGACGAGGACA |
| | | TCACCGGCCGGCTGAAGGACCGGGTGCAGCCCGAGATCCTGGAGG |
| | | CCCTGCTGAAGCACATCTCCTTCGACAAGTTCGTGCAGATCTCCCTG |
| | | AAGGCCCTGCGGCGGATCGTGCCCCTGATGGAGCAGGGCAAGCGG |
| | | TACGACGAGGCCTGCGCCGAGATCTACGGCGACCACTACGGCAAG |
| | | AAGAACACCGAGGAGAAGATCTACCTGCCCCCCATCCCCGCCGACG |
| | | AGATCCGGAACCCCGTGGTGCTGCGGGCCCTGTCCCAGGCCCGGAA |
| | | GGTGATCAACGGCGTGGTGCGGCGGTACGGCTCCCCCGCCCGGATC |
| | | CACATCGAGACCGCCCGGGAGGTGGGCAAGTCCTTCAAGGACCGG |
| | | AAGGAGATCGAGAAGCGGCAGGAGGAGAACCGGAAGGACCGGGA |
| | | GAAGGCCGCCGCCAAGTTCCGGGAGTACTTCCCCAACTTCGTGGGC |
| | | GAGCCCAAGTCCAAGGACATCCTGAAGCTGCGGCTGTACGAGCAGC |
| | | AGCACGGCAAGTGCCTGTACTCCGGCAAGGAGATCAACCTGGTGCG |
| | | GCTGAACGAGAAGGGCTACGTGGAGATCGACCACGCCCTGCCCTTC |
| | | TCCCGGACCTGGGACGACTCCTTCAACAACAAGGTGCTGGTGCTGG |
| | | GCTCCGAGAACCAGAACAAGGGCAACCAGACCCCCTACGAGTACTT |
| | | CAACGGCAAGGACAACTCCCGGGAGTGGCAGGAGTTCAAGGCCCG |
| | | GGTGGAGACCTCCCGGTTCCCCCGGTCCAAGAAGCAGCGGATCCTG |
| | | CTGCAGAAGTTCGACGAGGACGGCTTCAAGGAGTGCAACCTGAAC |
| | | GACACCCGGTACGTGAACCGGTTCCTGTGCCAGTTCGTGGCCGACC |
| | | ACATCCTGCTGACCGGCAAGGGCAAGCGGCGGGTGTTCGCCTCCAA |
| | | CGGCCAGATCACCAACCTGCTGCGGGGCTTCTGGGGCCTGCGGAAG |
| | | GTGCGGGCCGAGAACGACCGGCACCACGCCCTGGACGCCGTGGTG |
| | | GTGGCCTGCTCCACCGTGGCCATGCAGCAGAAGATCACCCGGTTCG |
| | | TGCGGTACAAGGAGATGAACGCCTTCGACGGCAAGACCATCGACA |
| | | AGGAGACCGGCAAGGTGCTGCACCAGAAGACCCACTTCCCCCAGCC |
| | | CTGGGAGTTCTTCGCCCAGGAGGTGATGATCCGGGTGTTCGGCAAG |
| | | CCCGACGGCAAGCCCGAGTTCGAGGAGGCCGACACCCCCGAGAAG |
| | | CTGCGGACCCTGCTGGCCGAGAAGCTGTCCTCCCGGCCCGAGGCCG |
| | | TGCACGAGTACGTGACCCCCCTGTTCGTGTCCCGGGCCCCCAACCG |
| | | GAAGATGTCCGGCGCCCACAAGGACACCCTGCGGTCCGCCAAGCG |
| | | GTTCGTGAAGCACAACGAGAAGATCTCCGTGAAGCGGGTGTGGCTG |
| | | ACCGAGATCAAGCTGGCCGACCTGGAGAACATGGTGAACTACAAG |
| | | AACGGCCGGGAGATCGAGCTGTACGAGGCCCTGAAGGCCCGGCTG |
| | | GAGGCCTACGGCGGCAACGCCAAGCAGGCCTTCGACCCCAAGGAC |
| | | AACCCCTTCTACAAGAAGGGCGGCCAGCTGGTGAAGGCCGTGCGG |
| | | GTGGAGAAGACCCAGGAGTCCGGCGTGCTGCTGAACAAGAAGAAC |
| | | GCCTACACCATCGCCGACAACGGCGACATGGTGCGGGTGGACGTGT |
| | | TCTGCAAGGTGGACAAGAAGGGCAAGAACCAGTACTTCATCGTGCC |
| | | CATCTACGCCTGGCAGGTGGCCGAGAACATCCTGCCCGACATCGAC |
| | | TGCAAGGGCTACCGGATCGACGACTCCTACACCTTCTGCTTCTCCCT |
| | | GCACAAGTACGACCTGATCGCCTTCCAGAAGGACGAGAAGTCCAA |
| | | GGTGGAGTTCGCCTACTACATCAACTGCGACTCCTCCAACGGCCGG |
| | | TTCTACCTGGCCTGGCACGACAAGGGCTCCAAGGAGCAGCAGTTCC |
| | | GGATCTCCACCCAGAACCTGGTGCTGATCCAGAAGTACCAGGTGAA |
| | | CGAGCTGGGCAAGGAGATCCGGCCCTGCCGGCTGAAGAAGCGGCC |
| | | CCCCGTGCGGTACCCCTACGACGTGCCCGACTACGCCGCCGCCCCC |
| | | GCCGCCAAGAAGAAGAAGCTGGACTAGCTAGCaccagcctcaagaacacccga |
| | | atggagtctctaagctacataataccaacttacactttacaaaatgttgtcccccaaaatgtagccattcgtatctgct |
| | | cctaataaaaagaaagtttcttcacattctCTCGAGAAAAAAAAAAAATGGAAAAAAA |
| | | AAAAACGGAAAAAAAAAAAAGGTAAAAAAAAAAAATATAAAAAA |
| | | AAAAAACATAAAAAAAAAAAACGAAAAAAAAAAAACGTAAAAAA |
| | | AAAAAACTCAAAAAAAAAAAAGATAAAAAAAAAAAACCTAAAAA |
| | | AAAAAAATGTAAAAAAAAAAAAGGGAAAAAAAAAAAACGCAAAA |
| | | AAAAAAAACACAAAAAAAAAAAATGCAAAAAAAAAAAATCGAAA |
| | | AAAAAAAAATCTAAAAAAAAAAAACGAAAAAAAAAAAACCCAAA |
| | | AAAAAAAAAGACAAAAAAAAAAAATAGAAAAAAAAAAAAGTTAA |
| | | AAAAAAAAAACTGAAAAAAAAAAAATTTAAAAAAAAAAAAT |
| 171 | mRNA J | GGGAAGCUCAGAAUAAACGCUCAACUUUGGCCGGAUCUGCCACCA |
| | encoding | UGGUGCCCAAGAAGAAGCGGAAGGUGGAGGACAAGCGGCCCGCC |
| | Nme2Cas9 | GCCACCAAGAAGGCCGGCCAGGCCAAGAAGAAGAAGAUGGCCGCC |
| | | UUCAAGCCCAACCCCAUCAACUACAUCCUGGGCCUGGACAUCGGC |
| | | AUCGCCUCCGUGGGCUGGGCCAUGGUGGAGAUCGACGAGGAGGA |
| | | GAACCCCAUCCGGCUGAUCGACCUGGGCGUGCGGGUGUUCGAGCG |
| | | GGCCGAGGUGCCCAAGACCGGCGACUCCCUGGCCAUGGCCCGGCG |
| | | GCUGGCCCGGUCCGUGCGGCGGCUGACCCGGCGGCGGGCCCACCG |
| | | GCUGCUGCGGGCCCGGCGGCUGCUGAAGCGGGAGGGCGUGCUGCA |
| | | GGCCGCCGACUUCGACGAGAACGGCCUGAUCAAGUCCCUGCCCAA |
| | | CACCCCCUGGCAGCUGCGGGCCGCCGCCCUGGACCGGAAGCUGAC |
| | | CCCCCUGGAGUGGUCCGCCGUGCUGCUGCACCUGAUCAAGCACCG |
| | | GGGCUACCUGUCCCAGCGGAAGAACGAGGGCGAGACCGCCGACAA |
| | | GGAGCUGGGCGCCCUGCUGAAGGGCGUGGCCAACAACGCCCACGC |
| | | CCUGCAGACCGGCGACUUCCGGACCCCCGCCGAGCUGGCCCUGAA |
| | | CAAGUUCGAGAAGGAGUCCGGCCACAUCCGGAACCAGCGGGGCGA |
| | | CUACUCCCACACCUUCUCCCGGAAGGACCUGCAGGCCGAGCUGAU |
| | | CCUGCUGUUCGAGAAGCAGAAGGAGUUCGGCAACCCCCACGUGUC |
| | | CGGCGGCCUGAAGGAGGGCAUCGAGACCCUGCUGAUGACCCAGCG |
| | | GCCCGCCCUGUCCGGCGACGCCGUGCAGAAGAUGCUGGGCCACUG |
| | | CACCUUCGAGCCCGCCGAGCCCAAGGCCGCCAAGAACACCUACAC |
| | | CGCCGAGCGGUUCAUCUGGCUGACCAAGCUGAACAACCUGCGGAU |
| | | CCUGGAGCAGGGCUCCGAGCGGCCCCUGACCGACACCGAGCGGGC |
| | | CACCCUGAUGGACGAGCCCUACCGGAAGUCCAAGCUGACCUACGC |
| | | CCAGGCCCGGAAGCUGCUGGGCCUGGAGGACACCGCCUUCUUCAA |
| | | GGGCCUGCGGUACGGCAAGGACAACGCCGAGGCCUCCACCCUGAU |
| | | GGAGAUGAAGGCCUACCACGCCAUCUCCCGGGCCCUGGAGAAGGA |
| | | GGGCCUGAAGGACAAGAAGUCCCCCCUGAACCUGUCCUCCGAGCU |
| | | GCAGGACGAGAUCGGCACCGCCUUCUCCCUGUUCAAGACCGACGA |
| | | GGACAUCACCGGCCGGCUGAAGGACCGGGUGCAGCCCGAGAUCCU |
| | | GGAGGCCCUGCUGAAGCACAUCUCCUUCGACAAGUUCGUGCAGAU |
| | | CUCCCUGAAGGCCCUGCGGCGGAUCGUGCCCCUGAUGGAGCAGGG |
| | | CAAGCGGUACGACGAGGCCUGCGCCGAGAUCUACGGCGACCACUA |
| | | CGGCAAGAAGAACACCGAGGAGAAGAUCUACCUGCCCCCCAUCCC |
| | | CGCCGACGAGAUCCGGAACCCCGUGGUGCUGCGGGCCCUGUCCCA |
| | | GGCCCGGAAGGUGAUCAACGGCGUGGUGCGGCGGUACGGCUCCCC |
| | | CGCCCGGAUCCACAUCGAGACCGCCCGGGAGGUGGGCAAGUCCUU |
| | | CAAGGACCGGAAGGAGAUCGAGAAGCGGCAGGAGGAGAACCGGA |
| | | AGGACCGGGAGAAGGCCGCCGCCAAGUUCCGGGAGUACUUCCCCA |
| | | ACUUCGUGGGCGAGCCCAAGUCCAAGGACAUCCUGAAGCUGCGGC |
| | | UGUACGAGCAGCAGCACGGCAAGUGCCUGUACUCCGGCAAGGAG |
| | | AUCAACCUGGUGCGGCUGAACGAGAAGGGCUACGUGGAGAUCGA |
| | | CCACGCCCUGCCCUUCUCCCGGACCUGGGACGACUCCUUCAACAA |
| | | CAAGGUGCUGGUGCUGGGCUCCGAGAACCAGAACAAGGGCAACC |
| | | AGACCCCCUACGAGUACUUCAACGGCAAGGACAACUCCCGGGAGU |
| | | GGCAGGAGUUCAAGGCCCGGGUGGAGACCUCCCGGUUCCCCCGGU |
| | | CCAAGAAGCAGCGGAUCCUGCUGCAGAAGUUCGACGAGGACGGC |
| | | UUCAAGGAGUGCAACCUGAACGACACCCGGUACGUGAACCGGUUC |
| | | CUGUGCCAGUUCGUGGCCGACCACAUCCUGCUGACCGGCAAGGGC |
| | | AAGCGGCGGGUGUUCGCCUCCAACGGCCAGAUCACCAACCUGCUG |
| | | CGGGGCUUCUGGGGCCUGCGGAAGGUGCGGGCCGAGAACGACCG |
| | | GCACCACGCCCUGGACGCCGUGGUGGUGGCCUGCUCCACCGUGGC |
| | | CAUGCAGCAGAAGAUCACCCGGUUCGUGCGGUACAAGGAGAUGA |
| | | ACGCCUUCGACGGCAAGACCAUCGACAAGGAGACCGGCAAGGUGC |
| | | UGCACCAGAAGACCCACUUCCCCCAGCCCUGGGAGUUCUUCGCCC |
| | | AGGAGGUGAUGAUCCGGGUGUUCGGCAAGCCCGACGGCAAGCCC |
| | | GAGUUCGAGGAGGCCGACACCCCCGAGAAGCUGCGGACCCUGCUG |
| | | GCCGAGAAGCUGUCCUCCCGGCCCGAGGCCGUGCACGAGUACGUG |
| | | ACCCCCCUGUUCGUGUCCCGGGCCCCCAACCGGAAGAUGUCCGGC |
| | | GCCCACAAGGACACCCUGCGGUCCGCCAAGCGGUUCGUGAAGCAC |
| | | AACGAGAAGAUCUCCGUGAAGCGGGUGUGGCUGACCGAGAUCAA |
| | | GCUGGCCGACCUGGAGAACAUGGUGAACUACAAGAACGGCCGGG |
| | | AGAUCGAGCUGUACGAGGCCCUGAAGGCCCGGCUGGAGGCCUACG |
| | | GCGGCAACGCCAAGCAGGCCUUCGACCCCAAGGACAACCCCUUCU |
| | | ACAAGAAGGGCGGCCAGCUGGUGAAGGCCGUGCGGGUGGAGAAG |
| | | ACCCAGGAGUCCGGCGUGCUGCUGAACAAGAAGAACGCCUACACC |
| | | AUCGCCGACAACGGCGACAUGGUGCGGGUGGACGUGUUCUGCAA |
| | | GGUGGACAAGAAGGGCAAGAACCAGUACUUCAUCGUGCCCAUCU |
| | | ACGCCUGGCAGGUGGCCGAGAACAUCCUGCCCGACAUCGACUGCA |
| | | AGGGCUACCGGAUCGACGACUCCUACACCUUCUGCUUCUCCCUGC |
| | | ACAAGUACGACCUGAUCGCCUUCCAGAAGGACGAGAAGUCCAAG |
| | | GUGGAGUUCGCCUACUACAUCAACUGCGACUCCUCCAACGGCCGG |
| | | UUCUACCUGGCCUGGCACGACAAGGGCUCCAAGGAGCAGCAGUUC |
| | | CGGAUCUCCACCCAGAACCUGGUGCUGAUCCAGAAGUACCAGGUG |
| | | AACGAGCUGGGCAAGGAGAUCCGGCCCUGCCGGCUGAAGAAGCG |
| | | GCCCCCCGUGCGGGAGGACAAGCGGCCCGCCGCCACCAAGAAGGC |
| | | CGGCCAGGCCAAGAAGAAGAAGUACCCCUACGACGUGCCCGACUA |
| | | CGCCGGCUACCCCUACGACGUGCCCGACUACGCCGGCUCCUACCC |
| | | CUACGACGUGCCCGACUACGCCGCCGCCCCCGCCGCCAAGAAGAA |
| | | GAAGCUGGACUAGCUAGCACCAGCCUCAAGAACACCCGAAUGGAG |
| | | UCUCUAAGCUACAUAAUACCAACUUACACUUUACAAAAUGUUGU |
| | | CCCCCAAAAUGUAGCCAUUCGUAUCUGCUCCUAAUAAAAAGAAA |
| | | GUUUCUUCACAUUCUCUCGAGAAAAAAAAAAAAUGGAAAAAAAA |
| | | AAAACGGAAAAAAAAAAAAGGUAAAAAAAAAAAAUAUAAAAAAA |
| | | AAAAACAUAAAAAAAAAAAACGAAAAAAAAAAAACGUAAAAAAA |
| | | AAAAACUCAAAAAAAAAAAAGAUAAAAAAAAAAAACCUAAAAAA |
| | | AAAAAAUGUAAAAAAAAAAAAGGGAAAAAAAAAAAACGCAAAAA |
| | | AAAAAAACACAAAAAAAAAAAAUGCAAAAAAAAAAAAUCGAAAA |
| | | AAAAAAAAUCUAAAAAAAAAAAACGAAAAAAAAAAAACCCAAAA |
| | | AAAAAAAAGACAAAAAAAAAAAAUAGAAAAAAAAAAAAGUUAAA |
| | | AAAAAAAAACUGAAAAAAAAAAAAUUUAAAAAAAAAAAAUCUAG |
| 172 | mRNA K | GGGaagctcagaataaacgctcaactttggccggatctgccacCatggccgccttcaagcccaaccccatca |
| | encoding | actacatcctgggcctggacatcggcatcgcctccgtgggctgggccatggtggagatcgacgaggaggaga |
| | Nme2Cas9 | accccatccggctgatcgacctgggcgtgcgggtgttcgagcgggccgaggtgcccaagaccggcgactccc |
| | | tggccatggcccggcggctggcccggtccgtgcggcggctgacccggcggcgggcccaccggctgctgcg |
| | | ggcccggcggctgctgaagcgggagggcgtgctgcaggccgccgacttcgacgagaacggcctgatcaagt |
| | | ccctgcccaacaccccctggcagctgcgggccgccgccctggaccggaagctgacccccctggagtggtcc |
| | | gccgtgctgctgcacctgatcaagcaccggggctacctgtcccagcggaagaacgagggcgagaccgccga |
| | | caaggagctgggcgccctgctgaagggcgtggccaacaacgcccacgccctgcagaccggcgacttccgga |
| | | cccccgccgagctggccctgaacaagttcgagaaggagtccggccacatccggaaccagcggggcgactac |
| | | tcccacaccttctcccggaaggacctgcaggccgagctgatcctgctgttcgagaagcagaaggagttcggca |
| | | acccccacgtgtccggcggcctgaaggagggcatcgagaccctgctgatgacccagcggcccgccctgtccg |
| | | gcgacgccgtgcagaagatgctgggccactgcaccttcgagcccgccgagcccaaggccgccaagaacacc |
| | | tacaccgccgagcggttcatctggctgaccaagctgaacaacctgcggatcctggagcagggctccgagcgg |
| | | cccctgaccgacaccgagcgggccaccctgatggacgagccctaccggaagtccaagctgacctacgccca |
| | | ggcccggaagctgctgggcctggaggacaccgccttcttcaagggcctgcggtacggcaaggacaacgccg |
| | | aggcctccaccctgatggagatgaaggcctaccacgccatctcccgggccctggagaaggagggcctgaagg |
| | | acaagaagtcccccctgaacctgtcctccgagctgcaggacgagatcggcaccgccttctccctgttcaagacc |
| | | gacgaggacatcaccggccggctgaaggaccgggtgcagcccgagatcctggaggccctgctgaagcacat |
| | | ctccttcgacaagttcgtgcagatctccctgaaggccctgcggcggatgtgcccctgatggagcagggcaagc |
| | | ggtacgacgaggcctgcgccgagatctacggcgaccactacggcaagaagaacaccgaggagaagatctac |
| | | ctgccccccatccccgccgacgagatccggaaccccgtggtgctggggccctgtcccaggcccggaaggtg |
| | | atcaacggcgtggtgcggcggtacggctcccccgcccggatccacatcgagaccgcccgggaggtgggcaa |
| | | gtccttcaaggaccggaaggagatcgagaagcggcaggaggagaaccggaaggaccgggagaaggccgc |
| | | cgccaagttccgggagtacttccccaacttcgtgggcgagcccaagtccaaggacatcctgaagctgcggctgt |
| | | acgagcagcagcacggcaagtgcctgtactccggcaaggagatcaacctggtgcggctgaacgagaagggc |
| | | tacgtggagatcgaccacgccctgcccttctcccggacctgggacgactccttcaacaacaaggtgctggtgct |
| | | gggctccgagaaccagaacaagggcaaccagaccccctacgagtacttcaacggcaaggacaactcccggg |
| | | agtggcaggagttcaaggcccgggtggagacctcccggttcccccggtccaagaagcagcggatcctgctgc |
| | | agaagttcgacgaggacggcttcaaggagtgcaacctgaacgacacccggtacgtgaaccggttcctgtgcca |
| | | gttcgtggccgaccacatcctgctgaccggcaagggcaagcggcgggtgttcgcctccaacggccagatcac |
| | | caacctgctgcggggcttctggggcctgcggaaggtgcgggccgagaacgaccggcaccacgccctggacg |
| | | ccgtggtggtggcctgctccaccgtggccatgcagcagaagatcacccggttcgtgcggtacaaggagatgaa |
| | | cgccttcgacggcaagaccatcgacaaggagaccggcaaggtgctgcaccagaagacccacttcccccagcc |
| | | ctgggagttcttcgcccaggaggtgatgatccgggtgttcggcaagcccgacggcaagcccgagttcgaggag |
| | | gccgacacccccgagaagctgcggaccctgctggccgagaagctgtcctcccggcccgaggccgtgcacga |
| | | gtacgtgacccccctgttcgtgtcccgggcccccaaccggaagatgtccggcgcccacaaggacaccctgcg |
| | | gtccgccaagcggttcgtgaagcacaacgagaagatctccgtgaagcgggtgtggctgaccgagatcaagctg |
| | | gccgacctggagaacatggtgaactacaagaacggccgggagatcgagctgtacgaggccctgaaggcccg |
| | | gctggaggcctacggcggcaacgccaagcaggccttcgaccccaaggacaaccccttctacaagaagggcg |
| | | gccagctggtgaaggccgtgcgggtggagaagacccaggagtccggcgtgctgctgaacaagaagaacgcc |
| | | tacaccatcgccgacaacggcgacatggtgcgggtggacgtgttctgcaaggtggacaagaagggcaagaac |
| | | cagtacttcatcgtgcccatctacgcctggcaggtggccgagaacatcctgcccgacatcgactgcaagggcta |
| | | ccggatcgacgactcctacaccttctgcttctccctgcacaagtacgacctgatcgccttccagaaggacgagaa |
| | | gtccaaggtggagttcgcctactacatcaactgcgactcctccaacggccggttctacctggcctggcacgacaa |
| | | gggctccaaggagcagcagttccggatctccacccagaacctggtgctgatccagaagtaccaggtgaacgag |
| | | ctgggcaaggagatccggccctgccggctgaagaagcggccccccgtgcggTCCGGAAAGCGG |
| | | ACCGCCGACGGCTCCGGAGGAGGAAGCCCCAAGAAGAAGCGGAAG |
| | | GTGtagctagcaccagcctcaagaacacccgaatggagtctctaagctacataataccaacttacactttacaa |
| | | aatgttgtcccccaaaatgtagccattcgtatctgctcctaataaaaagaaagtttcttcacattctctcgagAAA |
| | | AAAAAAAAATGGAAAAAAAAAAAACGGAAAAAAAAAAAAGGTAA |
| | | AAAAAAAAAATATAAAAAAAAAAAACATAAAAAAAAAAAACGAA |
| | | AAAAAAAAAACGTAAAAAAAAAAACTCAAAAAAAAAAAGATAAA |
| | | AAAAAAAAACCTAAAAAAAAAAAATGTAAAAAAAAAAAAGGGAA |
| | | AAAAAAAAAACGCAAAAAAAAAAAACACAAAAAAAAAAAATGCA |
| | | AAAAAAAAAAATCGAAAAAAAAAAAATCTAAAAAAAAAAAACGA |
| | | AAAAAAAAAAACCCAAAAAAAAAAAAGACAAAAAAAAAAAATAG |
| | | AAAAAAAAAAAGTTAAAAAAAAAAAACTGAAAAAAAAAAAATTTA |
| | | AAAAAAAAAAAT |
| 173 | mRNA L | GGGaagctcagaataaacgctcaactttggccggatctgccacCatgGACGGCTCCGGCGGC |
| | encoding | GGCTCCCCCAAGAAGAAGCGGAAGGTGGGCGGCTCCGGCGGCGGC |
| | Nme2Cas9 | gccgccttcaagcccaaccccatcaactacatcctgggcctggacatcggcatcgcctccgtgggctgggccat |
| | | ggtggagatcgacgaggaggagaaccccatccggctgatcgacctgggcgtgcgggtgttcgagcgggccg |
| | | aggtgcccaagaccggcgactccctggccatggcccggcggctggcccggtccgtgcggcggctgacccgg |
| | | cggcgggcccaccggctgctgcgggcccggcggctgctgaagcgggagggcgtgctgcaggccgccgact |
| | | tcgacgagaacggcctgatcaagtccctgcccaacaccccctggcagctgcgggccgccgccctggaccgga |
| | | agctgacccccctggagtggtccgccgtgctgctgcacctgatcaagcaccggggctacctgtcccagcggaa |
| | | gaacgagggcgagaccgccgacaaggagctgggcgccctgctgaagggcgtggccaacaacgcccacgc |
| | | cctgcagaccggcgacttccggacccccgccgagctggccctgaacaagttcgagaaggagtccggccacat |
| | | ccggaaccagcggggcgactactcccacaccttctcccggaaggacctgcaggccgagctgatcctgctgttc |
| | | gagaagcagaaggagttcggcaacccccacgtgtccggcggcctgaaggagggcatcgagaccctgctgat |
| | | gacccagcggcccgccctgtccggcgacgccgtgcagaagatgctgggccactgcaccttcgagcccgccg |
| | | agcccaaggccgccaagaacacctacaccgccgagcggttcatctggctgaccaagctgaacaacctgcgga |
| | | tcctggagcagggctccgagcggcccctgaccgacaccgagcgggccaccctgatggacgagccctaccgg |
| | | aagtccaagctgacctacgcccaggcccggaagctgctgggcctggaggacaccgccttcttcaagggcctgc |
| | | ggtacggcaaggacaacgccgaggcctccaccctgatggagatgaaggcctaccacgccatctcccgggccc |
| | | tggagaaggagggcctgaaggacaagaagtcccccctgaacctgtcctccgagctgcaggacgagatcggca |
| | | ccgccttctccctgttcaagaccgacgaggacatcaccggccggctgaaggaccgggtgcagcccgagatcct |
| | | ggaggccctgctgaagcacatctccttcgacaagttcgtgcagatctccctgaaggccctgcggcggatcgtgc |
| | | ccctgatggagcagggcaagcggtacgacgaggcctgcgccgagatctacggcgaccactacggcaagaag |
| | | aacaccgaggagaagatctacctgccccccatccccgccgacgagatccggaaccccgtggtgctgcgggcc |
| | | ctgtcccaggcccggaaggtgatcaacggcgtggtgcggcggtacggctcccccgcccggatccacatcgag |
| | | accgcccgggaggtgggcaagtccttcaaggaccggaaggagatcgagaagcggcaggaggagaaccgg |
| | | aaggaccgggagaaggccgccgccaagttccgggagtacttccccaacttcgtggggagcccaagtccaag |
| | | gacatcctgaagctgcggctgtacgagcagcagcacggcaagtgcctgtactccggcaaggagatcaacctg |
| | | gtgcggctgaacgagaagggctacgtggagatcgaccacgccctgcccttctcccggacctgggacgactcct |
| | | tcaacaacaaggtgctggtgctgggctccgagaaccagaacaagggcaaccagaccccctacgagtacttcaa |
| | | cggcaaggacaactcccgggagtggcaggagttcaaggcccgggtggagacctcccggttcccccggtcca |
| | | agaagcagcggatcctgctgcagaagttcgacgaggacggcttcaaggagtgcaacctgaacgacacccggt |
| | | acgtgaaccggttcctgtgccagttcgtggccgaccacatcctgctgaccggcaagggcaagcggcgggtgtt |
| | | cgcctccaacggccagatcaccaacctgctgcggggcttctggggcctgcggaaggtgcgggccgagaacg |
| | | accggcaccacgccctggacgccgtggtggtggcctgctccaccgtggccatgcagcagaagatcacccggt |
| | | tcgtgcggtacaaggagatgaacgccttcgacggcaagaccatcgacaaggagaccggcaaggtgctgcacc |
| | | agaagacccacttcccccagccctgggagttcttcgcccaggaggtgatgatccgggtgttcggcaagcccga |
| | | cggcaagcccgagttcgaggaggccgacacccccgagaagctgcggaccctgctggccgagaagctgtcct |
| | | cccggcccgaggccgtgcacgagtacgtgacccccctgttcgtgtcccgggcccccaaccggaagatgtccg |
| | | gcgcccacaaggacaccctgcggtccgccaagcggttcgtgaagcacaacgagaagatctccgtgaagcgg |
| | | gtgtggctgaccgagatcaagctggccgacctggagaacatggtgaactacaagaacggccgggagatcgag |
| | | ctgtacgaggccctgaaggcccggctggaggcctacggcggcaacgccaagcaggccttcgaccccaagga |
| | | caaccccttctacaagaagggcggccagctggtgaaggccgtgcgggtggagaagacccaggagtccggcg |
| | | tgctgctgaacaagaagaacgcctacaccatcgccgacaacggcgacatggtgcgggtggacgtgttctgcaa |
| | | ggtggacaagaagggcaagaaccagtacttcatcgtgcccatctacgcctggcaggtggccgagaacatcctg |
| | | cccgacatcgactgcaagggctaccggatcgacgactcctacaccttctgcttctccctgcacaagtacgacctg |
| | | atcgccttccagaaggacgagaagtccaaggtggagttcgcctactacatcaactgcgactcctccaacggccg |
| | | gttctacctggcctggcacgacaagggctccaaggagcagcagttccggatctccacccagaacctggtgctg |
| | | atccagaagtaccaggtgaacgagctgggcaaggagatccggccctgccggctgaagaagcggccccccgt |
| | | gcggtagctagcaccagcctcaagaacacccgaatggagtctctaagctacataataccaacttacactttacaa |
| | | aatgttgtcccccaaaatgtagccattcgtatctgctcctaataaaaagaaagtttcttcacattctctcgagAAA |
| | | AAAAAAAAATGGAAAAAAAAAAAACGGAAAAAAAAAAAAGGTAA |
| | | AAAAAAAAAATATAAAAAAAAAAAACATAAAAAAAAAAAACGAA |
| | | AAAAAAAAAACGTAAAAAAAAAAAACTCAAAAAAAAAAAGATAA |
| | | AAAAAAAAAACCTAAAAAAAAAAAATGTAAAAAAAAAAAAGGGA |
| | | AAAAAAAAAAACGCAAAAAAAAAAAACACAAAAAAAAAAAATGC |
| | | AAAAAAAAAAAATCGAAAAAAAAAAAATCTAAAAAAAAAAAACG |
| | | AAAAAAAAAAAACCCAAAAAAAAAAAAGACAAAAAAAAAAAATA |
| | | GAAAAAAAAAAAGTTAAAAAAAAAAAACTGAAAAAAAAAAAATTT |
| | | AAAAAAAAAAAAT |
| 174 | mRNA M | GGGAAGCUCAGAAUAAACGCUCAACUUUGGCCGGAUCUGCCACCA |
| | encoding | UGGACGGCUCCGGCGGCGGCUCCCCCAAGAAGAAGCGGAAGGUGG |
| | Nme2Cas9 with | GCGGCUCCGGCGGCGGCGCCGCCUUCAAGCCCAACCCCAUCAACU |
| | HiBiT tag | ACAUCCUGGGCCUGGACAUCGGCAUCGCCUCCGUGGGCUGGGCCA |
| | | UGGUGGAGAUCGACGAGGAGGAGAACCCCAUCCGGCUGAUCGAC |
| | | CUGGGCGUGCGGGUGUUCGAGCGGGCCGAGGUGCCCAAGACCGGC |
| | | GACUCCCUGGCCAUGGCCCGGCGGCUGGCCCGGUCCGUGCGGCGG |
| | | CUGACCCGGCGGCGGGCCCACCGGCUGCUGCGGGCCCGGCGGCUG |
| | | CUGAAGCGGGAGGGCGUGCUGCAGGCCGCCGACUUCGACGAGAAC |
| | | GGCCUGAUCAAGUCCCUGCCCAACACCCCCUGGCAGCUGCGGGCC |
| | | GCCGCCCUGGACCGGAAGCUGACCCCCCUGGAGUGGUCCGCCGUG |
| | | CUGCUGCACCUGAUCAAGCACCGGGGCUACCUGUCCCAGCGGAAG |
| | | AACGAGGGCGAGACCGCCGACAAGGAGCUGGGCGCCCUGCUGAAG |
| | | GGCGUGGCCAACAACGCCCACGCCCUGCAGACCGGCGACUUCCGG |
| | | ACCCCCGCCGAGCUGGCCCUGAACAAGUUCGAGAAGGAGUCCGGC |
| | | CACAUCCGGAACCAGCGGGGCGACUACUCCCACACCUUCUCCCGG |
| | | AAGGACCUGCAGGCCGAGCUGAUCCUGCUGUUCGAGAAGCAGAA |
| | | GGAGUUCGGCAACCCCCACGUGUCCGGCGGCCUGAAGGAGGGCAU |
| | | CGAGACCCUGCUGAUGACCCAGCGGCCCGCCCUGUCCGGCGACGC |
| | | CGUGCAGAAGAUGCUGGGCCACUGCACCUUCGAGCCCGCCGAGCC |
| | | CAAGGCCGCCAAGAACACCUACACCGCCGAGCGGUUCAUCUGGCU |
| | | GACCAAGCUGAACAACCUGCGGAUCCUGGAGCAGGGCUCCGAGCG |
| | | GCCCCUGACCGACACCGAGCGGGCCACCCUGAUGGACGAGCCCUA |
| | | CCGGAAGUCCAAGCUGACCUACGCCCAGGCCCGGAAGCUGCUGGG |
| | | CCUGGAGGACACCGCCUUCUUCAAGGGCCUGCGGUACGGCAAGGA |
| | | CAACGCCGAGGCCUCCACCCUGAUGGAGAUGAAGGCCUACCACGC |
| | | CAUCUCCCGGGCCCUGGAGAAGGAGGGCCUGAAGGACAAGAAGU |
| | | CCCCCCUGAACCUGUCCUCCGAGCUGCAGGACGAGAUCGGCACCG |
| | | CCUUCUCCCUGUUCAAGACCGACGAGGACAUCACCGGCCGGCUGA |
| | | AGGACCGGGUGCAGCCCGAGAUCCUGGAGGCCCUGCUGAAGCACA |
| | | UCUCCUUCGACAAGUUCGUGCAGAUCUCCCUGAAGGCCCUGCGGC |
| | | GGAUCGUGCCCCUGAUGGAGCAGGGCAAGCGGUACGACGAGGCC |
| | | UGCGCCGAGAUCUACGGCGACCACUACGGCAAGAAGAACACCGAG |
| | | GAGAAGAUCUACCUGCCCCCCAUCCCCGCCGACGAGAUCCGGAAC |
| | | CCCGUGGUGCUGCGGGCCCUGUCCCAGGCCCGGAAGGUGAUCAAC |
| | | GGCGUGGUGCGGCGGUACGGCUCCCCCGCCCGGAUCCACAUCGAG |
| | | ACCGCCCGGGAGGUGGGCAAGUCCUUCAAGGACCGGAAGGAGAU |
| | | CGAGAAGCGGCAGGAGGAGAACCGGAAGGACCGGGAGAAGGCCG |
| | | CCGCCAAGUUCCGGGAGUACUUCCCCAACUUCGUGGGCGAGCCCA |
| | | AGUCCAAGGACAUCCUGAAGCUGCGGCUGUACGAGCAGCAGCACG |
| | | GCAAGUGCCUGUACUCCGGCAAGGAGAUCAACCUGGUGCGGCUG |
| | | AACGAGAAGGGCUACGUGGAGAUCGACCACGCCCUGCCCUUCUCC |
| | | CGGACCUGGGACGACUCCUUCAACAACAAGGUGCUGGUGCUGGGC |
| | | UCCGAGAACCAGAACAAGGGCAACCAGACCCCCUACGAGUACUUC |
| | | AACGGCAAGGACAACUCCCGGGAGUGGCAGGAGUUCAAGGCCCG |
| | | GGUGGAGACCUCCCGGUUCCCCCGGUCCAAGAAGCAGCGGAUCCU |
| | | GCUGCAGAAGUUCGACGAGGACGGCUUCAAGGAGUGCAACCUGA |
| | | ACGACACCCGGUACGUGAACCGGUUCCUGUGCCAGUUCGUGGCCG |
| | | ACCACAUCCUGCUGACCGGCAAGGGCAAGCGGCGGGUGUUCGCCU |
| | | CCAACGGCCAGAUCACCAACCUGCUGCGGGGCUUCUGGGGCCUGC |
| | | GGAAGGUGCGGGCCGAGAACGACCGGCACCACGCCCUGGACGCCG |
| | | UGGUGGUGGCCUGCUCCACCGUGGCCAUGCAGCAGAAGAUCACCC |
| | | GGUUCGUGCGGUACAAGGAGAUGAACGCCUUCGACGGCAAGACC |
| | | AUCGACAAGGAGACCGGCAAGGUGCUGCACCAGAAGACCCACUUC |
| | | CCCCAGCCCUGGGAGUUCUUCGCCCAGGAGGUGAUGAUCCGGGUG |
| | | UUCGGCAAGCCCGACGGCAAGCCCGAGUUCGAGGAGGCCGACACC |
| | | CCCGAGAAGCUGCGGACCCUGCUGGCCGAGAAGCUGUCCUCCCGG |
| | | CCCGAGGCCGUGCACGAGUACGUGACCCCCCUGUUCGUGUCCCGG |
| | | GCCCCCAACCGGAAGAUGUCCGGCGCCCACAAGGACACCCUGCGG |
| | | UCCGCCAAGCGGUUCGUGAAGCACAACGAGAAGAUCUCCGUGAA |
| | | GCGGGUGUGGCUGACCGAGAUCAAGCUGGCCGACCUGGAGAACA |
| | | UGGUGAACUACAAGAACGGCCGGGAGAUCGAGCUGUACGAGGCC |
| | | CUGAAGGCCCGGCUGGAGGCCUACGGCGGCAACGCCAAGCAGGCC |
| | | UUCGACCCCAAGGACAACCCCUUCUACAAGAAGGGCGGCCAGCUG |
| | | GUGAAGGCCGUGCGGGUGGAGAAGACCCAGGAGUCCGGCGUGCU |
| | | GCUGAACAAGAAGAACGCCUACACCAUCGCCGACAACGGCGACAU |
| | | GGUGCGGGUGGACGUGUUCUGCAAGGUGGACAAGAAGGGCAAGA |
| | | ACCAGUACUUCAUCGUGCCCAUCUACGCCUGGCAGGUGGCCGAGA |
| | | ACAUCCUGCCCGACAUCGACUGCAAGGGCUACCGGAUCGACGACU |
| | | CCUACACCUUCUGCUUCUCCCUGCACAAGUACGACCUGAUCGCCU |
| | | UCCAGAAGGACGAGAAGUCCAAGGUGGAGUUCGCCUACUACAUC |
| | | AACUGCGACUCCUCCAACGGCCGGUUCUACCUGGCCUGGCACGAC |
| | | AAGGGCUCCAAGGAGCAGCAGUUCCGGAUCUCCACCCAGAACCUG |
| | | GUGCUGAUCCAGAAGUACCAGGUGAACGAGCUGGGCAAGGAGAU |
| | | CCGGCCCUGCCGGCUGAAGAAGCGGCCCCCCGUGCGGUCCGAGUC |
| | | CGCCACCCCCGAGUCCGUGUCCGGCUGGCGGCUGUUCAAGAAGAU |
| | | CUCCUAGCUAGCACCAGCCUCAAGAACACCCGAAUGGAGUCUCUA |
| | | AGCUACAUAAUACCAACUUACACUUUACAAAAUGUUGUCCCCCAA |
| | | AAUGUAGCCAUUCGUAUCUGCUCCUAAUAAAAAGAAAGUUUCUU |
| | | CACAUUCUCUCGAGAAAAAAAAAAAAUGGAAAAAAAAAAAACGG |
| | | AAAAAAAAAAAAGGUAAAAAAAAAAAAUAUAAAAAAAAAAAACA |
| | | UAAAAAAAAAAAACGAAAAAAAAAAAACGUAAAAAAAAAAAACU |
| | | CAAAAAAAAAAAAGAUAAAAAAAAAAAACCUAAAAAAAAAAAAU |
| | | GUAAAAAAAAAAAAGGGAAAAAAAAAAAACGCAAAAAAAAAAAA |
| | | CACAAAAAAAAAAAAUGCAAAAAAAAAAAAUCGAAAAAAAAAAA |
| | | AUCUAAAAAAAAAAAACGAAAAAAAAAAAACCCAAAAAAAAAAA |
| | | AGACAAAAAAAAAAAAUAGAAAAAAAAAAAAGUUAAAAAAAAAA |
| | | AACUGAAAAAAAAAAAAUUUAAAAAAAAAAAAUCUAG |
| 175 | mRNA N | GGGAAGCUCAGAAUAAACGCUCAACUUUGGCCGGAUCUGCCACCA |
| | encoding | UGGACGGCUCCGGCGGCGGCUCCCCCAAGAAGAAGCGGAAGGUGG |
| | Nme2Cas9 | GCGGCUCCGGCGGCGGCGCCGCCUUCAAGCCCAACCCCAUCAACU |
| | | ACAUCCUGGGCCUGGACAUCGGCAUCGCCUCCGUGGGCUGGGCCA |
| | | UGGUGGAGAUCGACGAGGAGGAGAACCCCAUCCGGCUGAUCGAC |
| | | CUGGGCGUGCGGGUGUUCGAGCGGGCCGAGGUGCCCAAGACCGGC |
| | | GACUCCCUGGCCAUGGCCCGGCGGCUGGCCCGGUCCGUGCGGCGG |
| | | CUGACCCGGCGGCGGGCCCACCGGCUGCUGCGGGCCCGGCGGCUG |
| | | CUGAAGCGGGAGGGCGUGCUGCAGGCCGCCGACUUCGACGAGAAC |
| | | GGCCUGAUCAAGUCCCUGCCCAACACCCCCUGGCAGCUGCGGGCC |
| | | GCCGCCCUGGACCGGAAGCUGACCCCCCUGGAGUGGUCCGCCGUG |
| | | CUGCUGCACCUGAUCAAGCACCGGGGCUACCUGUCCCAGCGGAAG |
| | | AACGAGGGCGAGACCGCCGACAAGGAGCUGGGCGCCCUGCUGAAG |
| | | GGCGUGGCCAACAACGCCCACGCCCUGCAGACCGGCGACUUCCGG |
| | | ACCCCCGCCGAGCUGGCCCUGAACAAGUUCGAGAAGGAGUCCGGC |
| | | CACAUCCGGAACCAGCGGGGCGACUACUCCCACACCUUCUCCCGG |
| | | AAGGACCUGCAGGCCGAGCUGAUCCUGCUGUUCGAGAAGCAGAA |
| | | GGAGUUCGGCAACCCCCACGUGUCCGGCGGCCUGAAGGAGGGCAU |
| | | CGAGACCCUGCUGAUGACCCAGCGGCCCGCCCUGUCCGGCGACGC |
| | | CGUGCAGAAGAUGCUGGGCCACUGCACCUUCGAGCCCGCCGAGCC |
| | | CAAGGCCGCCAAGAACACCUACACCGCCGAGCGGUUCAUCUGGCU |
| | | GACCAAGCUGAACAACCUGCGGAUCCUGGAGCAGGGCUCCGAGCG |
| | | GCCCCUGACCGACACCGAGCGGGCCACCCUGAUGGACGAGCCCUA |
| | | CCGGAAGUCCAAGCUGACCUACGCCCAGGCCCGGAAGCUGCUGGG |
| | | CCUGGAGGACACCGCCUUCUUCAAGGGCCUGCGGUACGGCAAGGA |
| | | CAACGCCGAGGCCUCCACCCUGAUGGAGAUGAAGGCCUACCACGC |
| | | CAUCUCCCGGGCCCUGGAGAAGGAGGGCCUGAAGGACAAGAAGU |
| | | CCCCCCUGAACCUGUCCUCCGAGCUGCAGGACGAGAUCGGCACCG |
| | | CCUUCUCCCUGUUCAAGACCGACGAGGACAUCACCGGCCGGCUGA |
| | | AGGACCGGGUGCAGCCCGAGAUCCUGGAGGCCCUGCUGAAGCACA |
| | | UCUCCUUCGACAAGUUCGUGCAGAUCUCCCUGAAGGCCCUGCGGC |
| | | GGAUCGUGCCCCUGAUGGAGCAGGGCAAGCGGUACGACGAGGCC |
| | | UGCGCCGAGAUCUACGGCGACCACUACGGCAAGAAGAACACCGAG |
| | | GAGAAGAUCUACCUGCCCCCCAUCCCCGCCGACGAGAUCCGGAAC |
| | | CCCGUGGUGCUGCGGGCCCUGUCCCAGGCCCGGAAGGUGAUCAAC |
| | | GGCGUGGUGCGGCGGUACGGCUCCCCCGCCCGGAUCCACAUCGAG |
| | | ACCGCCCGGGAGGUGGGCAAGUCCUUCAAGGACCGGAAGGAGAU |
| | | CGAGAAGCGGCAGGAGGAGAACCGGAAGGACCGGGAGAAGGCCG |
| | | CCGCCAAGUUCCGGGAGUACUUCCCCAACUUCGUGGGCGAGCCCA |
| | | AGUCCAAGGACAUCCUGAAGCUGCGGCUGUACGAGCAGCAGCACG |
| | | GCAAGUGCCUGUACUCCGGCAAGGAGAUCAACCUGGUGCGGCUG |
| | | AACGAGAAGGGCUACGUGGAGAUCGACCACGCCCUGCCCUUCUCC |
| | | CGGACCUGGGACGACUCCUUCAACAACAAGGUGCUGGUGCUGGGC |
| | | UCCGAGAACCAGAACAAGGGCAACCAGACCCCCUACGAGUACUUC |
| | | AACGGCAAGGACAACUCCCGGGAGUGGCAGGAGUUCAAGGCCCG |
| | | GGUGGAGACCUCCCGGUUCCCCCGGUCCAAGAAGCAGCGGAUCCU |
| | | GCUGCAGAAGUUCGACGAGGACGGCUUCAAGGAGUGCAACCUGA |
| | | ACGACACCCGGUACGUGAACCGGUUCCUGUGCCAGUUCGUGGCCG |
| | | ACCACAUCCUGCUGACCGGCAAGGGCAAGCGGCGGGUGUUCGCCU |
| | | CCAACGGCCAGAUCACCAACCUGCUGCGGGGCUUCUGGGGCCUGC |
| | | GGAAGGUGCGGGCCGAGAACGACCGGCACCACGCCCUGGACGCCG |
| | | UGGUGGUGGCCUGCUCCACCGUGGCCAUGCAGCAGAAGAUCACCC |
| | | GGUUCGUGCGGUACAAGGAGAUGAACGCCUUCGACGGCAAGACC |
| | | AUCGACAAGGAGACCGGCAAGGUGCUGCACCAGAAGACCCACUUC |
| | | CCCCAGCCCUGGGAGUUCUUCGCCCAGGAGGUGAUGAUCCGGGUG |
| | | UUCGGCAAGCCCGACGGCAAGCCCGAGUUCGAGGAGGCCGACACC |
| | | CCCGAGAAGCUGCGGACCCUGCUGGCCGAGAAGCUGUCCUCCCGG |
| | | CCCGAGGCCGUGCACGAGUACGUGACCCCCCUGUUCGUGUCCCGG |
| | | GCCCCCAACCGGAAGAUGUCCGGCGCCCACAAGGACACCCUGCGG |
| | | UCCGCCAAGCGGUUCGUGAAGCACAACGAGAAGAUCUCCGUGAA |
| | | GCGGGUGUGGCUGACCGAGAUCAAGCUGGCCGACCUGGAGAACA |
| | | UGGUGAACUACAAGAACGGCCGGGAGAUCGAGCUGUACGAGGCC |
| | | CUGAAGGCCCGGCUGGAGGCCUACGGCGGCAACGCCAAGCAGGCC |
| | | UUCGACCCCAAGGACAACCCCUUCUACAAGAAGGGCGGCCAGCUG |
| | | GUGAAGGCCGUGCGGGUGGAGAAGACCCAGGAGUCCGGCGUGCU |
| | | GCUGAACAAGAAGAACGCCUACACCAUCGCCGACAACGGCGACAU |
| | | GGUGCGGGUGGACGUGUUCUGCAAGGUGGACAAGAAGGGCAAGA |
| | | ACCAGUACUUCAUCGUGCCCAUCUACGCCUGGCAGGUGGCCGAGA |
| | | ACAUCCUGCCCGACAUCGACUGCAAGGGCUACCGGAUCGACGACU |
| | | CCUACACCUUCUGCUUCUCCCUGCACAAGUACGACCUGAUCGCCU |
| | | UCCAGAAGGACGAGAAGUCCAAGGUGGAGUUCGCCUACUACAUC |
| | | AACUGCGACUCCUCCAACGGCCGGUUCUACCUGGCCUGGCACGAC |
| | | AAGGGCUCCAAGGAGCAGCAGUUCCGGAUCUCCACCCAGAACCUG |
| | | GUGCUGAUCCAGAAGUACCAGGUGAACGAGCUGGGCAAGGAGAU |
| | | CCGGCCCUGCCGGCUGAAGAAGCGGCCCCCCGUGCGGUCCGGAAA |
| | | GCGGACCGCCGACGGCUCCGGAGGAGGAAGCCCCGCCGCCAAGAA |
| | | GAAGAAGCUGGACUAGCUAGCACCAGCCUCAAGAACACCCGAAUG |
| | | GAGUCUCUAAGCUACAUAAUACCAACUUACACUUUACAAAAUGU |
| | | UGUCCCCCAAAAUGUAGCCAUUCGUAUCUGCUCCUAAUAAAAAG |
| | | AAAGUUUCUUCACAUUCUCUCGAGAAAAAAAAAAAAUGGAAAAA |
| | | AAAAAAACGGAAAAAAAAAAAAGGUAAAAAAAAAAAAUAUAAAA |
| | | AAAAAAAACAUAAAAAAAAAAAACGAAAAAAAAAAAACGUAAAA |
| | | AAAAAAAACUCAAAAAAAAAAAGAUAAAAAAAAAAAACCUAAAA |
| | | AAAAAAAAUGUAAAAAAAAAAAAGGGAAAAAAAAAAAACGCAAA |
| | | AAAAAAAAACACAAAAAAAAAAAAUGCAAAAAAAAAAAAUCGAA |
| | | AAAAAAAAAAUCUAAAAAAAAAAAACGAAAAAAAAAAAACCCAA |
| | | AAAAAAAAAAGACAAAAAAAAAAAAUAGAAAAAAAAAAAGUUAA |
| | | AAAAAAAAAACUGAAAAAAAAAAAAUUUAAAAAAAAAAAAUCUA |
| | | G |
| 176 | mRNA O | GGGAAGCUCAGAAUAAACGCUCAACUUUGGCCGGAUCUGCCACCA |
| | encoding | UGGACGGCUCCGGCGGCGGCUCCCCCAAGAAGAAGCGGAAGGUGG |
| | Nme2Cas9 | AGGACAAGCGGCCCGCCGCCACCAAGAAGGCCGGCCAGGCCAAGA |
| | | AGAAGAAGGGCGGCUCCGGCGGCGGCGCCGCCUUCAAGCCCAACC |
| | | CCAUCAACUACAUCCUGGGCCUGGACAUCGGCAUCGCCUCCGUGG |
| | | GCUGGGCCAUGGUGGAGAUCGACGAGGAGGAGAACCCCAUCCGG |
| | | CUGAUCGACCUGGGCGUGCGGGUGUUCGAGCGGGCCGAGGUGCCC |
| | | AAGACCGGCGACUCCCUGGCCAUGGCCCGGCGGCUGGCCCGGUCC |
| | | GUGCGGCGGCUGACCCGGCGGCGGGCCCACCGGCUGCUGCGGGCC |
| | | CGGCGGCUGCUGAAGCGGGAGGGCGUGCUGCAGGCCGCCGACUUC |
| | | GACGAGAACGGCCUGAUCAAGUCCCUGCCCAACACCCCCUGGCAG |
| | | CUGCGGGCCGCCGCCCUGGACCGGAAGCUGACCCCCCUGGAGUGG |
| | | UCCGCCGUGCUGCUGCACCUGAUCAAGCACCGGGGCUACCUGUCC |
| | | CAGCGGAAGAACGAGGGCGAGACCGCCGACAAGGAGCUGGGCGCC |
| | | CUGCUGAAGGGCGUGGCCAACAACGCCCACGCCCUGCAGACCGGC |
| | | GACUUCCGGACCCCCGCCGAGCUGGCCCUGAACAAGUUCGAGAAG |
| | | GAGUCCGGCCACAUCCGGAACCAGCGGGGCGACUACUCCCACACC |
| | | UUCUCCCGGAAGGACCUGCAGGCCGAGCUGAUCCUGCUGUUCGAG |
| | | AAGCAGAAGGAGUUCGGCAACCCCCACGUGUCCGGCGGCCUGAAG |
| | | GAGGGCAUCGAGACCCUGCUGAUGACCCAGCGGCCCGCCCUGUCC |
| | | GGCGACGCCGUGCAGAAGAUGCUGGGCCACUGCACCUUCGAGCCC |
| | | GCCGAGCCCAAGGCCGCCAAGAACACCUACACCGCCGAGCGGUUC |
| | | AUCUGGCUGACCAAGCUGAACAACCUGCGGAUCCUGGAGCAGGGC |
| | | UCCGAGCGGCCCCUGACCGACACCGAGCGGGCCACCCUGAUGGAC |
| | | GAGCCCUACCGGAAGUCCAAGCUGACCUACGCCCAGGCCCGGAAG |
| | | CUGCUGGGCCUGGAGGACACCGCCUUCUUCAAGGGCCUGCGGUAC |
| | | GGCAAGGACAACGCCGAGGCCUCCACCCUGAUGGAGAUGAAGGCC |
| | | UACCACGCCAUCUCCCGGGCCCUGGAGAAGGAGGGCCUGAAGGAC |
| | | AAGAAGUCCCCCCUGAACCUGUCCUCCGAGCUGCAGGACGAGAUC |
| | | GGCACCGCCUUCUCCCUGUUCAAGACCGACGAGGACAUCACCGGC |
| | | CGGCUGAAGGACCGGGUGCAGCCCGAGAUCCUGGAGGCCCUGCUG |
| | | AAGCACAUCUCCUUCGACAAGUUCGUGCAGAUCUCCCUGAAGGCC |
| | | CUGCGGCGGAUCGUGCCCCUGAUGGAGCAGGGCAAGCGGUACGAC |
| | | GAGGCCUGCGCCGAGAUCUACGGCGACCACUACGGCAAGAAGAAC |
| | | ACCGAGGAGAAGAUCUACCUGCCCCCCAUCCCCGCCGACGAGAUC |
| | | CGGAACCCCGUGGUGCUGCGGGCCCUGUCCCAGGCCCGGAAGGUG |
| | | AUCAACGGCGUGGUGCGGCGGUACGGCUCCCCCGCCCGGAUCCAC |
| | | AUCGAGACCGCCCGGGAGGUGGGCAAGUCCUUCAAGGACCGGAA |
| | | GGAGAUCGAGAAGCGGCAGGAGGAGAACCGGAAGGACCGGGAGA |
| | | AGGCCGCCGCCAAGUUCCGGGAGUACUUCCCCAACUUCGUGGGCG |
| | | AGCCCAAGUCCAAGGACAUCCUGAAGCUGCGGCUGUACGAGCAGC |
| | | AGCACGGCAAGUGCCUGUACUCCGGCAAGGAGAUCAACCUGGUGC |
| | | GGCUGAACGAGAAGGGCUACGUGGAGAUCGACCACGCCCUGCCCU |
| | | UCUCCCGGACCUGGGACGACUCCUUCAACAACAAGGUGCUGGUGC |
| | | UGGGCUCCGAGAACCAGAACAAGGGCAACCAGACCCCCUACGAGU |
| | | ACUUCAACGGCAAGGACAACUCCCGGGAGUGGCAGGAGUUCAAG |
| | | GCCCGGGUGGAGACCUCCCGGUUCCCCCGGUCCAAGAAGCAGCGG |
| | | AUCCUGCUGCAGAAGUUCGACGAGGACGGCUUCAAGGAGUGCAA |
| | | CCUGAACGACACCCGGUACGUGAACCGGUUCCUGUGCCAGUUCGU |
| | | GGCCGACCACAUCCUGCUGACCGGCAAGGGCAAGCGGCGGGUGUU |
| | | CGCCUCCAACGGCCAGAUCACCAACCUGCUGCGGGGCUUCUGGGG |
| | | CCUGCGGAAGGUGCGGGCCGAGAACGACCGGCACCACGCCCUGGA |
| | | CGCCGUGGUGGUGGCCUGCUCCACCGUGGCCAUGCAGCAGAAGAU |
| | | CACCCGGUUCGUGCGGUACAAGGAGAUGAACGCCUUCGACGGCAA |
| | | GACCAUCGACAAGGAGACCGGCAAGGUGCUGCACCAGAAGACCCA |
| | | CUUCCCCCAGCCCUGGGAGUUCUUCGCCCAGGAGGUGAUGAUCCG |
| | | GGUGUUCGGCAAGCCCGACGGCAAGCCCGAGUUCGAGGAGGCCGA |
| | | CACCCCCGAGAAGCUGCGGACCCUGCUGGCCGAGAAGCUGUCCUC |
| | | CCGGCCCGAGGCCGUGCACGAGUACGUGACCCCCCUGUUCGUGUC |
| | | CCGGGCCCCCAACCGGAAGAUGUCCGGCGCCCACAAGGACACCCU |
| | | GCGGUCCGCCAAGCGGUUCGUGAAGCACAACGAGAAGAUCUCCGU |
| | | GAAGCGGGUGUGGCUGACCGAGAUCAAGCUGGCCGACCUGGAGA |
| | | ACAUGGUGAACUACAAGAACGGCCGGGAGAUCGAGCUGUACGAG |
| | | GCCCUGAAGGCCCGGCUGGAGGCCUACGGCGGCAACGCCAAGCAG |
| | | GCCUUCGACCCCAAGGACAACCCCUUCUACAAGAAGGGCGGCCAG |
| | | CUGGUGAAGGCCGUGCGGGUGGAGAAGACCCAGGAGUCCGGCGU |
| | | GCUGCUGAACAAGAAGAACGCCUACACCAUCGCCGACAACGGCGA |
| | | CAUGGUGCGGGUGGACGUGUUCUGCAAGGUGGACAAGAAGGGCA |
| | | AGAACCAGUACUUCAUCGUGCCCAUCUACGCCUGGCAGGUGGCCG |
| | | AGAACAUCCUGCCCGACAUCGACUGCAAGGGCUACCGGAUCGACG |
| | | ACUCCUACACCUUCUGCUUCUCCCUGCACAAGUACGACCUGAUCG |
| | | CCUUCCAGAAGGACGAGAAGUCCAAGGUGGAGUUCGCCUACUAC |
| | | AUCAACUGCGACUCCUCCAACGGCCGGUUCUACCUGGCCUGGCAC |
| | | GACAAGGGCUCCAAGGAGCAGCAGUUCCGGAUCUCCACCCAGAAC |
| | | CUGGUGCUGAUCCAGAAGUACCAGGUGAACGAGCUGGGCAAGGA |
| | | GAUCCGGCCCUGCCGGCUGAAGAAGCGGCCCCCCGUGCGGUAGCU |
| | | AGCACCAGCCUCAAGAACACCCGAAUGGAGUCUCUAAGCUACAUA |
| | | AUACCAACUUACACUUUACAAAAUGUUGUCCCCCAAAAUGUAGCC |
| | | AUUCGUAUCUGCUCCUAAUAAAAAGAAAGUUUCUUCACAUUCUC |
| | | UCGAGAAAAAAAAAAAAUGGAAAAAAAAAAAACGGAAAAAAAAA |
| | | AAAGGUAAAAAAAAAAAAUAUAAAAAAAAAAAACAUAAAAAAAA |
| | | AAAACGAAAAAAAAAAAACGUAAAAAAAAAAAACUCAAAAAAAA |
| | | AAAGAUAAAAAAAAAAAACCUAAAAAAAAAAAAUGUAAAAAAAA |
| | | AAAAGGGAAAAAAAAAAAACGCAAAAAAAAAAAACACAAAAAAA |
| | | AAAAAUGCAAAAAAAAAAAAUCGAAAAAAAAAAAAUCUAAAAAA |
| | | AAAAAACGAAAAAAAAAAAACCCAAAAAAAAAAAAGACAAAAAA |
| | | AAAAAAUAGAAAAAAAAAAAGUUAAAAAAAAAAAACUGAAAAAA |
| | | AAAAAAUUUAAAAAAAAAAAAUCUAG |
| 177 | mRNA P | GGGAAGCUCAGAAUAAACGCUCAACUUUGGCCGGAUCUGCCACCA |
| | encoding | UGGACGGCUCCGGCGGCGGCUCCCCCAAGAAGAAGCGGAAGGUGG |
| | Nme2Cas9 with | AGGACAAGCGGCCCGCCGCCACCAAGAAGGCCGGCCAGGCCAAGA |
| | HiBiT tag | AGAAGAAGGGCGGCUCCGGCGGCGGCGCCGCCUUCAAGCCCAACC |
| | | CCAUCAACUACAUCCUGGGCCUGGACAUCGGCAUCGCCUCCGUGG |
| | | GCUGGGCCAUGGUGGAGAUCGACGAGGAGGAGAACCCCAUCCGG |
| | | CUGAUCGACCUGGGCGUGCGGGUGUUCGAGCGGGCCGAGGUGCCC |
| | | AAGACCGGCGACUCCCUGGCCAUGGCCCGGCGGCUGGCCCGGUCC |
| | | GUGCGGCGGCUGACCCGGCGGCGGGCCCACCGGCUGCUGCGGGCC |
| | | CGGCGGCUGCUGAAGCGGGAGGGCGUGCUGCAGGCCGCCGACUUC |
| | | GACGAGAACGGCCUGAUCAAGUCCCUGCCCAACACCCCCUGGCAG |
| | | CUGCGGGCCGCCGCCCUGGACCGGAAGCUGACCCCCCUGGAGUGG |
| | | UCCGCCGUGCUGCUGCACCUGAUCAAGCACCGGGGCUACCUGUCC |
| | | CAGCGGAAGAACGAGGGCGAGACCGCCGACAAGGAGCUGGGCGCC |
| | | CUGCUGAAGGGCGUGGCCAACAACGCCCACGCCCUGCAGACCGGC |
| | | GACUUCCGGACCCCCGCCGAGCUGGCCCUGAACAAGUUCGAGAAG |
| | | GAGUCCGGCCACAUCCGGAACCAGCGGGGCGACUACUCCCACACC |
| | | UUCUCCCGGAAGGACCUGCAGGCCGAGCUGAUCCUGCUGUUCGAG |
| | | AAGCAGAAGGAGUUCGGCAACCCCCACGUGUCCGGCGGCCUGAAG |
| | | GAGGGCAUCGAGACCCUGCUGAUGACCCAGCGGCCCGCCCUGUCC |
| | | GGCGACGCCGUGCAGAAGAUGCUGGGCCACUGCACCUUCGAGCCC |
| | | GCCGAGCCCAAGGCCGCCAAGAACACCUACACCGCCGAGCGGUUC |
| | | AUCUGGCUGACCAAGCUGAACAACCUGCGGAUCCUGGAGCAGGGC |
| | | UCCGAGCGGCCCCUGACCGACACCGAGCGGGCCACCCUGAUGGAC |
| | | GAGCCCUACCGGAAGUCCAAGCUGACCUACGCCCAGGCCCGGAAG |
| | | CUGCUGGGCCUGGAGGACACCGCCUUCUUCAAGGGCCUGCGGUAC |
| | | GGCAAGGACAACGCCGAGGCCUCCACCCUGAUGGAGAUGAAGGCC |
| | | UACCACGCCAUCUCCCGGGCCCUGGAGAAGGAGGGCCUGAAGGAC |
| | | AAGAAGUCCCCCCUGAACCUGUCCUCCGAGCUGCAGGACGAGAUC |
| | | GGCACCGCCUUCUCCCUGUUCAAGACCGACGAGGACAUCACCGGC |
| | | CGGCUGAAGGACCGGGUGCAGCCCGAGAUCCUGGAGGCCCUGCUG |
| | | AAGCACAUCUCCUUCGACAAGUUCGUGCAGAUCUCCCUGAAGGCC |
| | | CUGCGGCGGAUCGUGCCCCUGAUGGAGCAGGGCAAGCGGUACGAC |
| | | GAGGCCUGCGCCGAGAUCUACGGCGACCACUACGGCAAGAAGAAC |
| | | ACCGAGGAGAAGAUCUACCUGCCCCCCAUCCCCGCCGACGAGAUC |
| | | CGGAACCCCGUGGUGCUGCGGGCCCUGUCCCAGGCCCGGAAGGUG |
| | | AUCAACGGCGUGGUGCGGCGGUACGGCUCCCCCGCCCGGAUCCAC |
| | | AUCGAGACCGCCCGGGAGGUGGGCAAGUCCUUCAAGGACCGGAA |
| | | GGAGAUCGAGAAGCGGCAGGAGGAGAACCGGAAGGACCGGGAGA |
| | | AGGCCGCCGCCAAGUUCCGGGAGUACUUCCCCAACUUCGUGGGCG |
| | | AGCCCAAGUCCAAGGACAUCCUGAAGCUGCGGCUGUACGAGCAGC |
| | | AGCACGGCAAGUGCCUGUACUCCGGCAAGGAGAUCAACCUGGUGC |
| | | GGCUGAACGAGAAGGGCUACGUGGAGAUCGACCACGCCCUGCCCU |
| | | UCUCCCGGACCUGGGACGACUCCUUCAACAACAAGGUGCUGGUGC |
| | | UGGGCUCCGAGAACCAGAACAAGGGCAACCAGACCCCCUACGAGU |
| | | ACUUCAACGGCAAGGACAACUCCCGGGAGUGGCAGGAGUUCAAG |
| | | GCCCGGGUGGAGACCUCCCGGUUCCCCCGGUCCAAGAAGCAGCGG |
| | | AUCCUGCUGCAGAAGUUCGACGAGGACGGCUUCAAGGAGUGCAA |
| | | CCUGAACGACACCCGGUACGUGAACCGGUUCCUGUGCCAGUUCGU |
| | | GGCCGACCACAUCCUGCUGACCGGCAAGGGCAAGCGGCGGGUGUU |
| | | CGCCUCCAACGGCCAGAUCACCAACCUGCUGCGGGGCUUCUGGGG |
| | | CCUGCGGAAGGUGCGGGCCGAGAACGACCGGCACCACGCCCUGGA |
| | | CGCCGUGGUGGUGGCCUGCUCCACCGUGGCCAUGCAGCAGAAGAU |
| | | CACCCGGUUCGUGCGGUACAAGGAGAUGAACGCCUUCGACGGCAA |
| | | GACCAUCGACAAGGAGACCGGCAAGGUGCUGCACCAGAAGACCCA |
| | | CUUCCCCCAGCCCUGGGAGUUCUUCGCCCAGGAGGUGAUGAUCCG |
| | | GGUGUUCGGCAAGCCCGACGGCAAGCCCGAGUUCGAGGAGGCCGA |
| | | CACCCCCGAGAAGCUGCGGACCCUGCUGGCCGAGAAGCUGUCCUC |
| | | CCGGCCCGAGGCCGUGCACGAGUACGUGACCCCCCUGUUCGUGUC |
| | | CCGGGCCCCCAACCGGAAGAUGUCCGGCGCCCACAAGGACACCCU |
| | | GCGGUCCGCCAAGCGGUUCGUGAAGCACAACGAGAAGAUCUCCGU |
| | | GAAGCGGGUGUGGCUGACCGAGAUCAAGCUGGCCGACCUGGAGA |
| | | ACAUGGUGAACUACAAGAACGGCCGGGAGAUCGAGCUGUACGAG |
| | | GCCCUGAAGGCCCGGCUGGAGGCCUACGGCGGCAACGCCAAGCAG |
| | | GCCUUCGACCCCAAGGACAACCCCUUCUACAAGAAGGGCGGCCAG |
| | | CUGGUGAAGGCCGUGCGGGUGGAGAAGACCCAGGAGUCCGGCGU |
| | | GCUGCUGAACAAGAAGAACGCCUACACCAUCGCCGACAACGGCGA |
| | | CAUGGUGCGGGUGGACGUGUUCUGCAAGGUGGACAAGAAGGGCA |
| | | AGAACCAGUACUUCAUCGUGCCCAUCUACGCCUGGCAGGUGGCCG |
| | | AGAACAUCCUGCCCGACAUCGACUGCAAGGGCUACCGGAUCGACG |
| | | ACUCCUACACCUUCUGCUUCUCCCUGCACAAGUACGACCUGAUCG |
| | | CCUUCCAGAAGGACGAGAAGUCCAAGGUGGAGUUCGCCUACUAC |
| | | AUCAACUGCGACUCCUCCAACGGCCGGUUCUACCUGGCCUGGCAC |
| | | GACAAGGGCUCCAAGGAGCAGCAGUUCCGGAUCUCCACCCAGAAC |
| | | CUGGUGCUGAUCCAGAAGUACCAGGUGAACGAGCUGGGCAAGGA |
| | | GAUCCGGCCCUGCCGGCUGAAGAAGCGGCCCCCCGUGCGGUCCGA |
| | | GUCCGCCACCCCCGAGUCCGUGUCCGGCUGGCGGCUGUUCAAGAA |
| | | GAUCUCCUAGCUAGCACCAGCCUCAAGAACACCCGAAUGGAGUCU |
| | | CUAAGCUACAUAAUACCAACUUACACUUUACAAAAUGUUGUCCCC |
| | | CAAAAUGUAGCCAUUCGUAUCUGCUCCUAAUAAAAAGAAAGUUU |
| | | CUUCACAUUCUCUCGAGAAAAAAAAAAAAUGGAAAAAAAAAAAA |
| | | CGGAAAAAAAAAAAAGGUAAAAAAAAAAAAUAUAAAAAAAAAAA |
| | | ACAUAAAAAAAAAAAACGAAAAAAAAAAAACGUAAAAAAAAAAA |
| | | ACUCAAAAAAAAAAAAGAUAAAAAAAAAAAACCUAAAAAAAAAA |
| | | AAUGUAAAAAAAAAAAAGGGAAAAAAAAAAAACGCAAAAAAAAA |
| | | AAACACAAAAAAAAAAAAUGCAAAAAAAAAAAAUCGAAAAAAAA |
| | | AAAAUCUAAAAAAAAAAAACGAAAAAAAAAAAACCCAAAAAAAA |
| | | AAAAGACAAAAAAAAAAAAUAGAAAAAAAAAAAAGUUAAAAAAA |
| | | AAAAACUGAAAAAAAAAAAAUUUAAAAAAAAAAAAUCUAG |
| 178 | mRNA Q | GGGAAGCUCAGAAUAAACGCUCAACUUUGGCCGGAUCUGCCACCA |
| | encoding | UGGACGGCUCCGGCGGCGGCUCCCCCAAGAAGAAGCGGAAGGUGG |
| | Nme2Cas9 | GCGGCUCCGGCGGCGGCGCCGCCUUCAAGCCCAACCCCAUCAACU |
| | | ACAUCCUGGGCCUGGACAUCGGCAUCGCCUCCGUGGGCUGGGCCA |
| | | UGGUGGAGAUCGACGAGGAGGAGAACCCCAUCCGGCUGAUCGAC |
| | | CUGGGCGUGCGGGUGUUCGAGCGGGCCGAGGUGCCCAAGACCGGC |
| | | GACUCCCUGGCCAUGGCCCGGCGGCUGGCCCGGUCCGUGCGGCGG |
| | | CUGACCCGGCGGCGGGCCCACCGGCUGCUGCGGGCCCGGCGGCUG |
| | | CUGAAGCGGGAGGGCGUGCUGCAGGCCGCCGACUUCGACGAGAAC |
| | | GGCCUGAUCAAGUCCCUGCCCAACACCCCCUGGCAGCUGCGGGCC |
| | | GCCGCCCUGGACCGGAAGCUGACCCCCCUGGAGUGGUCCGCCGUG |
| | | CUGCUGCACCUGAUCAAGCACCGGGGCUACCUGUCCCAGCGGAAG |
| | | AACGAGGGCGAGACCGCCGACAAGGAGCUGGGCGCCCUGCUGAAG |
| | | GGCGUGGCCAACAACGCCCACGCCCUGCAGACCGGCGACUUCCGG |
| | | ACCCCCGCCGAGCUGGCCCUGAACAAGUUCGAGAAGGAGUCCGGC |
| | | CACAUCCGGAACCAGCGGGGCGACUACUCCCACACCUUCUCCCGG |
| | | AAGGACCUGCAGGCCGAGCUGAUCCUGCUGUUCGAGAAGCAGAA |
| | | GGAGUUCGGCAACCCCCACGUGUCCGGCGGCCUGAAGGAGGGCAU |
| | | CGAGACCCUGCUGAUGACCCAGCGGCCCGCCCUGUCCGGCGACGC |
| | | CGUGCAGAAGAUGCUGGGCCACUGCACCUUCGAGCCCGCCGAGCC |
| | | CAAGGCCGCCAAGAACACCUACACCGCCGAGCGGUUCAUCUGGCU |
| | | GACCAAGCUGAACAACCUGCGGAUCCUGGAGCAGGGCUCCGAGCG |
| | | GCCCCUGACCGACACCGAGCGGGCCACCCUGAUGGACGAGCCCUA |
| | | CCGGAAGUCCAAGCUGACCUACGCCCAGGCCCGGAAGCUGCUGGG |
| | | CCUGGAGGACACCGCCUUCUUCAAGGGCCUGCGGUACGGCAAGGA |
| | | CAACGCCGAGGCCUCCACCCUGAUGGAGAUGAAGGCCUACCACGC |
| | | CAUCUCCCGGGCCCUGGAGAAGGAGGGCCUGAAGGACAAGAAGU |
| | | CCCCCCUGAACCUGUCCUCCGAGCUGCAGGACGAGAUCGGCACCG |
| | | CCUUCUCCCUGUUCAAGACCGACGAGGACAUCACCGGCCGGCUGA |
| | | AGGACCGGGUGCAGCCCGAGAUCCUGGAGGCCCUGCUGAAGCACA |
| | | UCUCCUUCGACAAGUUCGUGCAGAUCUCCCUGAAGGCCCUGCGGC |
| | | GGAUCGUGCCCCUGAUGGAGCAGGGCAAGCGGUACGACGAGGCC |
| | | UGCGCCGAGAUCUACGGCGACCACUACGGCAAGAAGAACACCGAG |
| | | GAGAAGAUCUACCUGCCCCCCAUCCCCGCCGACGAGAUCCGGAAC |
| | | CCCGUGGUGCUGCGGGCCCUGUCCCAGGCCCGGAAGGUGAUCAAC |
| | | GGCGUGGUGCGGCGGUACGGCUCCCCCGCCCGGAUCCACAUCGAG |
| | | ACCGCCCGGGAGGUGGGCAAGUCCUUCAAGGACCGGAAGGAGAU |
| | | CGAGAAGCGGCAGGAGGAGAACCGGAAGGACCGGGAGAAGGCCG |
| | | CCGCCAAGUUCCGGGAGUACUUCCCCAACUUCGUGGGCGAGCCCA |
| | | AGUCCAAGGACAUCCUGAAGCUGCGGCUGUACGAGCAGCAGCACG |
| | | GCAAGUGCCUGUACUCCGGCAAGGAGAUCAACCUGGUGCGGCUG |
| | | AACGAGAAGGGCUACGUGGAGAUCGACCACGCCCUGCCCUUCUCC |
| | | CGGACCUGGGACGACUCCUUCAACAACAAGGUGCUGGUGCUGGGC |
| | | UCCGAGAACCAGAACAAGGGCAACCAGACCCCCUACGAGUACUUC |
| | | AACGGCAAGGACAACUCCCGGGAGUGGCAGGAGUUCAAGGCCCG |
| | | GGUGGAGACCUCCCGGUUCCCCCGGUCCAAGAAGCAGCGGAUCCU |
| | | GCUGCAGAAGUUCGACGAGGACGGCUUCAAGGAGUGCAACCUGA |
| | | ACGACACCCGGUACGUGAACCGGUUCCUGUGCCAGUUCGUGGCCG |
| | | ACCACAUCCUGCUGACCGGCAAGGGCAAGCGGCGGGUGUUCGCCU |
| | | CCAACGGCCAGAUCACCAACCUGCUGCGGGGCUUCUGGGGCCUGC |
| | | GGAAGGUGCGGGCCGAGAACGACCGGCACCACGCCCUGGACGCCG |
| | | UGGUGGUGGCCUGCUCCACCGUGGCCAUGCAGCAGAAGAUCACCC |
| | | GGUUCGUGCGGUACAAGGAGAUGAACGCCUUCGACGGCAAGACC |
| | | AUCGACAAGGAGACCGGCAAGGUGCUGCACCAGAAGACCCACUUC |
| | | CCCCAGCCCUGGGAGUUCUUCGCCCAGGAGGUGAUGAUCCGGGUG |
| | | UUCGGCAAGCCCGACGGCAAGCCCGAGUUCGAGGAGGCCGACACC |
| | | CCCGAGAAGCUGCGGACCCUGCUGGCCGAGAAGCUGUCCUCCCGG |
| | | CCCGAGGCCGUGCACGAGUACGUGACCCCCCUGUUCGUGUCCCGG |
| | | GCCCCCAACCGGAAGAUGUCCGGCGCCCACAAGGACACCCUGCGG |
| | | UCCGCCAAGCGGUUCGUGAAGCACAACGAGAAGAUCUCCGUGAA |
| | | GCGGGUGUGGCUGACCGAGAUCAAGCUGGCCGACCUGGAGAACA |
| | | UGGUGAACUACAAGAACGGCCGGGAGAUCGAGCUGUACGAGGCC |
| | | CUGAAGGCCCGGCUGGAGGCCUACGGCGGCAACGCCAAGCAGGCC |
| | | UUCGACCCCAAGGACAACCCCUUCUACAAGAAGGGCGGCCAGCUG |
| | | GUGAAGGCCGUGCGGGUGGAGAAGACCCAGGAGUCCGGCGUGCU |
| | | GCUGAACAAGAAGAACGCCUACACCAUCGCCGACAACGGCGACAU |
| | | GGUGCGGGUGGACGUGUUCUGCAAGGUGGACAAGAAGGGCAAGA |
| | | ACCAGUACUUCAUCGUGCCCAUCUACGCCUGGCAGGUGGCCGAGA |
| | | ACAUCCUGCCCGACAUCGACUGCAAGGGCUACCGGAUCGACGACU |
| | | CCUACACCUUCUGCUUCUCCCUGCACAAGUACGACCUGAUCGCCU |
| | | UCCAGAAGGACGAGAAGUCCAAGGUGGAGUUCGCCUACUACAUC |
| | | AACUGCGACUCCUCCAACGGCCGGUUCUACCUGGCCUGGCACGAC |
| | | AAGGGCUCCAAGGAGCAGCAGUUCCGGAUCUCCACCCAGAACCUG |
| | | GUGCUGAUCCAGAAGUACCAGGUGAACGAGCUGGGCAAGGAGAU |
| | | CCGGCCCUGCCGGCUGAAGAAGCGGCCCCCCGUGCGGUAGCUAGC |
| | | ACCAGCCUCAAGAACACCCGAAUGGAGUCUCUAAGCUACAUAAUA |
| | | CCAACUUACACUUUACAAAAUGUUGUCCCCCAAAAUGUAGCCAUU |
| | | CGUAUCUGCUCCUAAUAAAAAGAAAGUUUCUUCACAUUCUCUCG |
| | | AGAAAAAAAAAAAAUGGAAAAAAAAAAAACGGAAAAAAAAAAAA |
| | | GGUAAAAAAAAAAAAUAUAAAAAAAAAAAACAUAAAAAAAAAAA |
| | | ACGAAAAAAAAAAAACGUAAAAAAAAAAAACUCAAAAAAAAAAA |
| | | GAUAAAAAAAAAAAACCUAAAAAAAAAAAAUGUAAAAAAAAAAA |
| | | AGGGAAAAAAAAAAAACGCAAAAAAAAAAAACACAAAAAAAAAA |
| | | AAUGCAAAAAAAAAAAAUCGAAAAAAAAAAAAUCUAAAAAAAAA |
| | | AAACGAAAAAAAAAAAACCCAAAAAAAAAAAAGACAAAAAAAAA |
| | | AAAUAGAAAAAAAAAAAGUUAAAAAAAAAAAACUGAAAAAAAAA |
| | | AAAUUUAAAAAAAAAAAAUCUAG |
| 179 | mRNA S | GGGAAGCUCAGAAUAAACGCUCAACUUUGGCCGGAUCUGCCACCA |
| | encoding | UGGACGGCUCCGGCGGCGGCUCCCCCAAGAAGAAGCGGAAGGUGG |
| | Nme2Cas9 base | AGGACAAGCGGCCCGCCGCCACCAAGAAGGCCGGCCAGGCCAAGA |
| | editor | AGAAGAAGGGCGGCUCCGGCGGCGGCGAGGCCUCCCCCGCCUCCG |
| | | GCCCCCGGCACCUGAUGGACCCCCACAUCUUCACCUCCAACUUCA |
| | | ACAACGGCAUCGGCCGGCACAAGACCUACCUGUGCUACGAGGUGG |
| | | AGCGGCUGGACAACGGCACCUCCGUGAAGAUGGACCAGCACCGGG |
| | | GCUUCCUGCACAACCAGGCCAAGAACCUGCUGUGCGGCUUCUACG |
| | | GCCGGCACGCCGAGCUGCGGUUCCUGGACCUGGUGCCCUCCCUGC |
| | | AGCUGGACCCCGCCCAGAUCUACCGGGUGACCUGGUUCAUCUCCU |
| | | GGUCCCCCUGCUUCUCCUGGGGCUGCGCCGGCGAGGUGCGGGCCU |
| | | UCCUGCAGGAGAACACCCACGUGCGGCUGCGGAUCUUCGCCGCCC |
| | | GGAUCUACGACUACGACCCCCUGUACAAGGAGGCCCUGCAGAUGC |
| | | UGCGGGACGCCGGCGCCCAGGUGUCCAUCAUGACCUACGACGAGU |
| | | UCAAGCACUGCUGGGACACCUUCGUGGACCACCAGGGCUGCCCCU |
| | | UCCAGCCCUGGGACGGCCUGGACGAGCACUCCCAGGCCCUGUCCG |
| | | GCCGGCUGCGGGCCAUCCUGCAGAACCAGGGCAACUCCGGCUCCG |
| | | AGACCCCCGGCACCUCCGAGUCCGCCACCCCCGAGUCCGCAGCGU |
| | | UCAAACCAAAUCCCAUCAACUACAUCCUGGGCCUGGCCAUCGGCA |
| | | UCGCCUCCGUGGGCUGGGCCAUGGUGGAGAUCGACGAGGAGGAG |
| | | AACCCCAUCCGGCUGAUCGACCUGGGCGUGCGGGUGUUCGAGCGG |
| | | GCCGAGGUGCCCAAGACCGGCGACUCCCUGGCCAUGGCCCGGCGG |
| | | CUGGCCCGGUCCGUGCGGCGGCUGACCCGGCGGCGGGCCCACCGG |
| | | CUGCUGCGGGCCCGGCGGCUGCUGAAGCGGGAGGGCGUGCUGCAG |
| | | GCCGCCGACUUCGACGAGAACGGCCUGAUCAAGUCCCUGCCCAAC |
| | | ACCCCCUGGCAGCUGCGGGCCGCCGCCCUGGACCGGAAGCUGACC |
| | | CCCCUGGAGUGGUCCGCCGUGCUGCUGCACCUGAUCAAGCACCGG |
| | | GGCUACCUGUCCCAGCGGAAGAACGAGGGCGAGACCGCCGACAAG |
| | | GAGCUGGGCGCCCUGCUGAAGGGCGUGGCCAACAACGCCCACGCC |
| | | CUGCAGACCGGCGACUUCCGGACCCCCGCCGAGCUGGCCCUGAAC |
| | | AAGUUCGAGAAGGAGUCCGGCCACAUCCGGAACCAGCGGGGCGAC |
| | | UACUCCCACACCUUCUCCCGGAAGGACCUGCAGGCCGAGCUGAUC |
| | | CUGCUGUUCGAGAAGCAGAAGGAGUUCGGCAACCCCCACGUGUCC |
| | | GGCGGCCUGAAGGAGGGCAUCGAGACCCUGCUGAUGACCCAGCGG |
| | | CCCGCCCUGUCCGGCGACGCCGUGCAGAAGAUGCUGGGCCACUGC |
| | | ACCUUCGAGCCCGCCGAGCCCAAGGCCGCCAAGAACACCUACACC |
| | | GCCGAGCGGUUCAUCUGGCUGACCAAGCUGAACAACCUGCGGAUC |
| | | CUGGAGCAGGGCUCCGAGCGGCCCCUGACCGACACCGAGCGGGCC |
| | | ACCCUGAUGGACGAGCCCUACCGGAAGUCCAAGCUGACCUACGCC |
| | | CAGGCCCGGAAGCUGCUGGGCCUGGAGGACACCGCCUUCUUCAAG |
| | | GGCCUGCGGUACGGCAAGGACAACGCCGAGGCCUCCACCCUGAUG |
| | | GAGAUGAAGGCCUACCACGCCAUCUCCCGGGCCCUGGAGAAGGAG |
| | | GGCCUGAAGGACAAGAAGUCCCCCCUGAACCUGUCCUCCGAGCUG |
| | | CAGGACGAGAUCGGCACCGCCUUCUCCCUGUUCAAGACCGACGAG |
| | | GACAUCACCGGCCGGCUGAAGGACCGGGUGCAGCCCGAGAUCCUG |
| | | GAGGCCCUGCUGAAGCACAUCUCCUUCGACAAGUUCGUGCAGAUC |
| | | UCCCUGAAGGCCCUGCGGCGGAUCGUGCCCCUGAUGGAGCAGGGC |
| | | AAGCGGUACGACGAGGCCUGCGCCGAGAUCUACGGCGACCACUAC |
| | | GGCAAGAAGAACACCGAGGAGAAGAUCUACCUGCCCCCCAUCCCC |
| | | GCCGACGAGAUCCGGAACCCCGUGGUGCUGCGGGCCCUGUCCCAG |
| | | GCCCGGAAGGUGAUCAACGGCGUGGUGCGGCGGUACGGCUCCCCC |
| | | GCCCGGAUCCACAUCGAGACCGCCCGGGAGGUGGGCAAGUCCUUC |
| | | AAGGACCGGAAGGAGAUCGAGAAGCGGCAGGAGGAGAACCGGAA |
| | | GGACCGGGAGAAGGCCGCCGCCAAGUUCCGGGAGUACUUCCCCAA |
| | | CUUCGUGGGCGAGCCCAAGUCCAAGGACAUCCUGAAGCUGCGGCU |
| | | GUACGAGCAGCAGCACGGCAAGUGCCUGUACUCCGGCAAGGAGA |
| | | UCAACCUGGUGCGGCUGAACGAGAAGGGCUACGUGGAGAUCGAC |
| | | CACGCCCUGCCCUUCUCCCGGACCUGGGACGACUCCUUCAACAAC |
| | | AAGGUGCUGGUGCUGGGCUCCGAGAACCAGAACAAGGGCAACCA |
| | | GACCCCCUACGAGUACUUCAACGGCAAGGACAACUCCCGGGAGUG |
| | | GCAGGAGUUCAAGGCCCGGGUGGAGACCUCCCGGUUCCCCCGGUC |
| | | CAAGAAGCAGCGGAUCCUGCUGCAGAAGUUCGACGAGGACGGCU |
| | | UCAAGGAGUGCAACCUGAACGACACCCGGUACGUGAACCGCUUCC |
| | | UGUGCCAGUUCGUGGCCGACCACAUCCUGCUGACCGGCAAGGGCA |
| | | AGCGGCGGGUGUUCGCCUCCAACGGCCAGAUCACCAACCUGCUGC |
| | | GGGGCUUCUGGGGCCUGCGGAAGGUGCGGGCCGAGAACGACCGG |
| | | CACCACGCCCUGGACGCCGUGGUGGUGGCCUGCUCCACCGUGGCC |
| | | AUGCAGCAGAAGAUCACCCGGUUCGUGCGGUACAAGGAGAUGAA |
| | | CGCCUUCGACGGCAAGACCAUCGACAAGGAGACCGGCAAGGUGCU |
| | | GCACCAGAAGACCCACUUCCCCCAGCCCUGGGAGUUCUUCGCCCA |
| | | GGAGGUGAUGAUCCGGGUGUUCGGCAAGCCCGACGGCAAGCCCG |
| | | AGUUCGAGGAGGCCGACACCCCCGAGAAGCUGCGGACCCUGCUGG |
| | | CCGAGAAGCUGUCCUCCCGGCCCGAGGCCGUGCACGAGUACGUGA |
| | | CCCCCCUGUUCGUGUCCCGGGCCCCCAACCGGAAGAUGUCCGGCG |
| | | CCCACAAGGACACCCUGCGGUCCGCCAAGCGGUUCGUGAAGCACA |
| | | ACGAGAAGAUCUCCGUGAAGCGGGUGUGGCUGACCGAGAUCAAG |
| | | CUGGCCGACCUGGAGAACAUGGUGAACUACAAGAACGGCCGGGA |
| | | GAUCGAGCUGUACGAGGCCCUGAAGGCCCGGCUGGAGGCCUACGG |
| | | CGGCAACGCCAAGCAGGCCUUCGACCCCAAGGACAACCCCUUCUA |
| | | CAAGAAGGGCGGCCAGCUGGUGAAGGCCGUGCGGGUGGAGAAGA |
| | | CCCAGGAGUCCGGCGUGCUGCUGAACAAGAAGAACGCCUACACCA |
| | | UCGCCGACAACGGCGACAUGGUGCGGGUGGACGUGUUCUGCAAG |
| | | GUGGACAAGAAGGGCAAGAACCAGUACUUCAUCGUGCCCAUCUA |
| | | CGCCUGGCAGGUGGCCGAGAACAUCCUGCCCGACAUCGACUGCAA |
| | | GGGCUACCGGAUCGACGACUCCUACACCUUCUGCUUCUCCCUGCA |
| | | CAAGUACGACCUGAUCGCCUUCCAGAAGGACGAGAAGUCCAAGG |
| | | UGGAGUUCGCCUACUACAUCAACUGCGACUCCUCCAACGGCCGGU |
| | | UCUACCUGGCCUGGCACGACAAGGGCUCCAAGGAGCAGCAGUUCC |
| | | GGAUCUCCACCCAGAACCUGGUGCUGAUCCAGAAGUACCAGGUGA |
| | | ACGAGCUGGGCAAGGAGAUCCGGCCCUGCCGGCUGAAGAAGCGGC |
| | | CCCCCGUGCGGUAGUGACUAGCACCAGCCUCAAGAACACCCGAAU |
| | | GGAGUCUCUAAGCUACAUAAUACCAACUUACACUUUACAAAAUG |
| | | UUGUCCCCCAAAAUGUAGCCAUUCGUAUCUGCUCCUAAUAAAAA |
| | | GAAAGUUUCUUCACAUUCUCUCGAGAAAAAAAAAAAAUGGAAAA |
| | | AAAAAAAACGGAAAAAAAAAAAGGUAAAAAAAAAAAAUAUAAAA |
| | | AAAAAAACAUAAAAAAAAAAAACGAAAAAAAAAAAACGUAAAAA |
| | | AAAAAAACUCAAAAAAAAAAAGAUAAAAAAAAAAAACCUAAAAA |
| | | AAAAAAAUGUAAAAAAAAAAAAGGGAAAAAAAAAAACGCAAAAA |
| | | AAAAAAACACAAAAAAAAAAAAUGCAAAAAAAAAAAAUCGAAAA |
| | | AAAAAAAAUCUAAAAAAAAAAAACGAAAAAAAAAAAACCCAAAA |
| | | AAAAAAAAGACAAAAAAAAAAAAUAGAAAAAAAAAAAGUUAAAA |
| | | AAAAAAAACUGAAAAAAAAAAAAUUUAAAAAAAAAAAAUCUAG |
| 180 | Open reading | atgaccggtgccgccttcaagcccaaccccatcaactacatcctgggcctggacatcggcatcgcctccgtggg |
| | frame for | ctgggccatggtggagatcgacgaggaggagaaccccatccggctgatcgacctgggcgtgcgggtgttcga |
| | Nme2Cas9 | gcgggccgaggtgcccaagaccggcgactccctggccatggcccggcggctggcccggtccgtgcggcgg |
| | encoded by | ctgacccggcggcgggcccaccggctgctgcgggcccggcggctgctgaagcgggagggcgtgctgcagg |
| | mRNA C | ccgccgacttcgacgagaacggcctgatcaagtccctgcccaacaccccctggcagctgcgggccgccgccc |
| | | tggaccggaagctgacccccctggagtggtccgccgtgctgctgcacctgatcaagcaccggggctacctgtc |
| | | ccagcggaagaacgagggcgagaccgccgacaaggagctgggcgccctgctgaagggcgtggccaacaa |
| | | cgcccacgccctgcagaccggcgacttccggacccccgccgagctggccctgaacaagttcgagaaggagtc |
| | | cggccacatccggaaccagcggggcgactactcccacaccttctcccggaaggacctgcaggccgagctgat |
| | | cctgctgttcgagaagcagaaggagttcggcaacccccacgtgtccggcggcctgaaggagggcatcgagac |
| | | cctgctgatgacccagcggcccgccctgtccggcgacgccgtgcagaagatgctgggccactgcaccttcga |
| | | gcccgccgagcccaaggccgccaagaacacctacaccgccgagcggttcatctggctgaccaagctgaacaa |
| | | cctgcggatcctggagcagggctccgagcggcccctgaccgacaccgagcgggccaccctgatggacgagc |
| | | cctaccggaagtccaagctgacctacgcccaggcccggaagctgctgggcctggaggacaccgccttcttcaa |
| | | gggcctgcggtacggcaaggacaacgccgaggcctccaccctgatggagatgaaggcctaccacgccatctc |
| | | ccgggccctggagaaggagggcctgaaggacaagaagtcccccctgaacctgtcctccgagctgcaggacg |
| | | agatcggcaccgccttctccctgttcaagaccgacgaggacatcaccggccggctgaaggaccgggtgcagc |
| | | ccgagatcctggaggccctgctgaagcacatctccttcgacaagttcgtgcagatctccctgaaggccctgcgg |
| | | cggatcgtgcccctgatggagcagggcaagcggtacgacgaggcctgcgccgagatctacggcgaccacta |
| | | cggcaagaagaacaccgaggagaagatctacctgccccccatccccgccgacgagatccggaaccccgtgg |
| | | tgctgcgggccctgtcccaggcccggaaggtgatcaacggcgtggtgcggcggtacggctcccccgcccgg |
| | | atccacatcgagaccgcccgggaggtgggcaagtccttcaaggaccggaaggagatcgagaagcggcagga |
| | | ggagaaccggaaggaccgggagaaggccgccgccaagttccgggagtacttccccaacttcgtgggcgagc |
| | | ccaagtccaaggacatcctgaagctgcggctgtacgagcagcagcacggcaagtgcctgtactccggcaagg |
| | | agatcaacctggtgcggctgaacgagaagggctacgtggagatcgaccacgccctgcccttctcccggacctg |
| | | ggacgactccttcaacaacaaggtgctggtgctgggctccgagaaccagaacaagggcaaccagacccccta |
| | | cgagtacttcaacggcaaggacaactcccgggagtggcaggagttcaaggcccgggtggagacctcccggtt |
| | | cccccggtccaagaagcagcggatcctgctgcagaagttcgacgaggacggcttcaaggagtgcaacctgaa |
| | | cgacacccggtacgtgaaccgcttcctgtgccagttcgtggccgaccacatcctgctgaccggcaagggcaag |
| | | cggcgggtgttcgcctccaacggccagatcaccaacctgctgcggggcttctggggcctgcggaaggtgcgg |
| | | gccgagaacgaccggcaccacgccctggacgccgtggtggtggcctgctccaccgtggccatgcagcagaa |
| | | gatcacccggttcgtgcggtacaaggagatgaacgccttcgacggcaagaccatcgacaaggagaccggcaa |
| | | ggtgctgcaccagaagacccacttcccccagccctgggagttcttcgcccaggaggtgatgatccgggtgttcg |
| | | gcaagcccgacggcaagcccgagttcgaggaggccgacacccccgagaagctgcggaccctgctggccga |
| | | gaagctgtcctcccggcccgaggccgtgcacgagtacgtgacccccctgttcgtgtcccgggcccccaaccgg |
| | | aagatgtccggcgcccacaaggacaccctgcggtccgccaagcggttcgtgaagcacaacgagaagatctcc |
| | | gtgaagcgggtgtggctgaccgagatcaagctggccgacctggagaacatggtgaactacaagaacggccgg |
| | | gagatcgagctgtacgaggccctgaaggcccggctggaggcctacggcggcaacgccaagcaggccttcga |
| | | ccccaaggacaaccccttctacaagaagggcggccagctggtgaaggccgtgcgggtggagaagacccagg |
| | | agtccggcgtgctgctgaacaagaagaacgcctacaccatcgccgacaacggcgacatggtgcgggtggacg |
| | | tgttctgcaaggtggacaagaagggcaagaaccagtacttcatcgtgcccatctacgcctggcaggtggccgag |
| | | aacatcctgcccgacatcgactgcaagggctaccggatcgacgactcctacaccttctgcttctccctgcacaag |
| | | tacgacctgatcgccttccagaaggacgagaagtccaaggtggagttcgcctactacatcaactgcgactcctcc |
| | | aacggccggttctacctggcctggcacgacaagggctccaaggagcagcagttccggatctccacccagaac |
| | | ctggtgctgatccagaagtaccaggtgaacgagctgggcaaggagatccggccctgccggctgaagaagcgg |
| | | ccccccgtgcggtccggaaagcggaccgccgacggctccgagttcgagtcccccaagaagaagcggaaggt |
| | | ggagtag |
| 181 | Open reading | ATGGCCGCCTTCAAGCCCAACCCCATCAACTACATCCTGGGCCTGG |
| | frame for | ACATCGGCATCGCCTCCGTGGGCTGGGCCATGGTGGAGATCGACGA |
| | Nme2Cas9 | GGAGGAGAACCCCATCCGGCTGATCGACCTGGGCGTGCGGGTGTTC |
| | encoded by | GAGCGGGCCGAGGTGCCCAAGACCGGCGACTCCCTGGCCATGGCCC |
| | mRNA H | GGCGGCTGGCCCGGTCCGTGCGGCGGCTGACCCGGCGGCGGGCCCA |
| | | CCGGCTGCTGCGGGCCCGGCGGCTGCTGAAGCGGGAGGGCGTGCTG |
| | | CAGGCCGCCGACTTCGACGAGAACGGCCTGATCAAGTCCCTGCCCA |
| | | ACACCCCCTGGCAGCTGCGGGCCGCCGCCCTGGACCGGAAGCTGAC |
| | | CCCCCTGGAGTGGTCCGCCGTGCTGCTGCACCTGATCAAGCACCGG |
| | | GGCTACCTGTCCCAGCGGAAGAACGAGGGCGAGACCGCCGACAAG |
| | | GAGCTGGGCGCCCTGCTGAAGGGCGTGGCCAACAACGCCCACGCCC |
| | | TGCAGACCGGCGACTTCCGGACCCCCGCCGAGCTGGCCCTGAACAA |
| | | GTTCGAGAAGGAGTCCGGCCACATCCGGAACCAGCGGGGCGACTA |
| | | CTCCCACACCTTCTCCCGGAAGGACCTGCAGGCCGAGCTGATCCTG |
| | | CTGTTCGAGAAGCAGAAGGAGTTCGGCAACCCCCACGTGTCCGGCG |
| | | GCCTGAAGGAGGGCATCGAGACCCTGCTGATGACCCAGCGGCCCGC |
| | | CCTGTCCGGCGACGCCGTGCAGAAGATGCTGGGCCACTGCACCTTC |
| | | GAGCCCGCCGAGCCCAAGGCCGCCAAGAACACCTACACCGCCGAG |
| | | CGGTTCATCTGGCTGACCAAGCTGAACAACCTGCGGATCCTGGAGC |
| | | AGGGCTCCGAGCGGCCCCTGACCGACACCGAGCGGGCCACCCTGAT |
| | | GGACGAGCCCTACCGGAAGTCCAAGCTGACCTACGCCCAGGCCCGG |
| | | AAGCTGCTGGGCCTGGAGGACACCGCCTTCTTCAAGGGCCTGCGGT |
| | | ACGGCAAGGACAACGCCGAGGCCTCCACCCTGATGGAGATGAAGG |
| | | CCTACCACGCCATCTCCCGGGCCCTGGAGAAGGAGGGCCTGAAGGA |
| | | CAAGAAGTCCCCCCTGAACCTGTCCTCCGAGCTGCAGGACGAGATC |
| | | GGCACCGCCTTCTCCCTGTTCAAGACCGACGAGGACATCACCGGCC |
| | | GGCTGAAGGACCGGGTGCAGCCCGAGATCCTGGAGGCCCTGCTGA |
| | | AGCACATCTCCTTCGACAAGTTCGTGCAGATCTCCCTGAAGGCCCT |
| | | GCGGCGGATCGTGCCCCTGATGGAGCAGGGCAAGCGGTACGACGA |
| | | GGCCTGCGCCGAGATCTACGGCGACCACTACGGCAAGAAGAACAC |
| | | CGAGGAGAAGATCTACCTGCCCCCCATCCCCGCCGACGAGATCCGG |
| | | AACCCCGTGGTGCTGCGGGCCCTGTCCCAGGCCCGGAAGGTGATCA |
| | | ACGGCGTGGTGCGGCGGTACGGCTCCCCCGCCCGGATCCACATCGA |
| | | GACCGCCCGGGAGGTGGGCAAGTCCTTCAAGGACCGGAAGGAGAT |
| | | CGAGAAGCGGCAGGAGGAGAACCGGAAGGACCGGGAGAAGGCCG |
| | | CCGCCAAGTTCCGGGAGTACTTCCCCAACTTCGTGGGCGAGCCCAA |
| | | GTCCAAGGACATCCTGAAGCTGCGGCTGTACGAGCAGCAGCACGGC |
| | | AAGTGCCTGTACTCCGGCAAGGAGATCAACCTGGTGCGGCTGAACG |
| | | AGAAGGGCTACGTGGAGATCGACCACGCCCTGCCCTTCTCCCGGAC |
| | | CTGGGACGACTCCTTCAACAACAAGGTGCTGGTGCTGGGCTCCGAG |
| | | AACCAGAACAAGGGCAACCAGACCCCCTACGAGTACTTCAACGGC |
| | | AAGGACAACTCCCGGGAGTGGCAGGAGTTCAAGGCCCGGGTGGAG |
| | | ACCTCCCGGTTCCCCCGGTCCAAGAAGCAGCGGATCCTGCTGCAGA |
| | | AGTTCGACGAGGACGGCTTCAAGGAGTGCAACCTGAACGACACCC |
| | | GGTACGTGAACCGGTTCCTGTGCCAGTTCGTGGCCGACCACATCCT |
| | | GCTGACCGGCAAGGGCAAGCGGCGGGTGTTCGCCTCCAACGGCCA |
| | | GATCACCAACCTGCTGCGGGGCTTCTGGGGCCTGCGGAAGGTGCGG |
| | | GCCGAGAACGACCGGCACCACGCCCTGGACGCCGTGGTGGTGGCCT |
| | | GCTCCACCGTGGCCATGCAGCAGAAGATCACCCGGTTCGTGCGGTA |
| | | CAAGGAGATGAACGCCTTCGACGGCAAGACCATCGACAAGGAGAC |
| | | CGGCAAGGTGCTGCACCAGAAGACCCACTTCCCCCAGCCCTGGGAG |
| | | TTCTTCGCCCAGGAGGTGATGATCCGGGTGTTCGGCAAGCCCGACG |
| | | GCAAGCCCGAGTTCGAGGAGGCCGACACCCCCGAGAAGCTGCGGA |
| | | CCCTGCTGGCCGAGAAGCTGTCCTCCCGGCCCGAGGCCGTGCACGA |
| | | GTACGTGACCCCCCTGTTCGTGTCCCGGGCCCCCAACCGGAAGATG |
| | | TCCGGCGCCCACAAGGACACCCTGCGGTCCGCCAAGCGGTTCGTGA |
| | | AGCACAACGAGAAGATCTCCGTGAAGCGGGTGTGGCTGACCGAGA |
| | | TCAAGCTGGCCGACCTGGAGAACATGGTGAACTACAAGAACGGCC |
| | | GGGAGATCGAGCTGTACGAGGCCCTGAAGGCCCGGCTGGAGGCCT |
| | | ACGGCGGCAACGCCAAGCAGGCCTTCGACCCCAAGGACAACCCCTT |
| | | CTACAAGAAGGGCGGCCAGCTGGTGAAGGCCGTGCGGGTGGAGAA |
| | | GACCCAGGAGTCCGGCGTGCTGCTGAACAAGAAGAACGCCTACAC |
| | | CATCGCCGACAACGGCGACATGGTGCGGGTGGACGTGTTCTGCAAG |
| | | GTGGACAAGTCCGGCGGCGGCTCCCCCAAGAAGAAGCGGAAGGTG |
| | | TCCGGCGGCTCCGGCAAGAACCAGTACTTCATCGTGCCCATCTACG |
| | | CCTGGCAGGTGGCCGAGAACATCCTGCCCGACATCGACTGCAAGGG |
| | | CTACCGGATCGACGACTCCTACACCTTCTGCTTCTCCCTGCACAAGT |
| | | ACGACCTGATCGCCTTCCAGAAGGACGAGAAGTCCAAGGTGGAGTT |
| | | CGCCTACTACATCAACTGCGACTCCTCCAACGGCCGGTTCTACCTGG |
| | | CCTGGCACGACAAGGGCTCCAAGGAGCAGCAGTTCCGGATCTCCAC |
| | | CCAGAACCTGGTGCTGATCCAGAAGTACCAGGTGAACGAGCTGGGC |
| | | AAGGAGATCCGGCCCTGCCGGCTGAAGAAGCGGCCCCCCGTGCGGT |
| | | AG |
| 182 | Open reading | ATGGTGCCCAAGAAGAAGCGGAAGGTGGCCGCCTTCAAGCCCAAC |
| | frame for | CCCATCAACTACATCCTGGGCCTGGACATCGGCATCGCCTCCGTGG |
| | Nme2Cas9 | GCTGGGCCATGGTGGAGATCGACGAGGAGGAGAACCCCATCCGGC |
| | encoded by | TGATCGACCTGGGCGTGCGGGTGTTCGAGCGGGCCGAGGTGCCCAA |
| | mRNA I | GACCGGCGACTCCCTGGCCATGGCCCGGCGGCTGGCCCGGTCCGTG |
| | | CGGCGGCTGACCCGGCGGCGGGCCCACCGGCTGCTGCGGGCCCGGC |
| | | GGCTGCTGAAGCGGGAGGGCGTGCTGCAGGCCGCCGACTTCGACG |
| | | AGAACGGCCTGATCAAGTCCCTGCCCAACACCCCCTGGCAGCTGCG |
| | | GGCCGCCGCCCTGGACCGGAAGCTGACCCCCCTGGAGTGGTCCGCC |
| | | GTGCTGCTGCACCTGATCAAGCACCGGGGCTACCTGTCCCAGCGGA |
| | | AGAACGAGGGCGAGACCGCCGACAAGGAGCTGGGCGCCCTGCTGA |
| | | AGGGCGTGGCCAACAACGCCCACGCCCTGCAGACCGGCGACTTCCG |
| | | GACCCCCGCCGAGCTGGCCCTGAACAAGTTCGAGAAGGAGTCCGGC |
| | | CACATCCGGAACCAGCGGGGCGACTACTCCCACACCTTCTCCCGGA |
| | | AGGACCTGCAGGCCGAGCTGATCCTGCTGTTCGAGAAGCAGAAGG |
| | | AGTTCGGCAACCCCCACGTGTCCGGCGGCCTGAAGGAGGGCATCGA |
| | | GACCCTGCTGATGACCCAGCGGCCCGCCCTGTCCGGCGACGCCGTG |
| | | CAGAAGATGCTGGGCCACTGCACCTTCGAGCCCGCCGAGCCCAAGG |
| | | CCGCCAAGAACACCTACACCGCCGAGCGGTTCATCTGGCTGACCAA |
| | | GCTGAACAACCTGCGGATCCTGGAGCAGGGCTCCGAGCGGCCCCTG |
| | | ACCGACACCGAGCGGGCCACCCTGATGGACGAGCCCTACCGGAAG |
| | | TCCAAGCTGACCTACGCCCAGGCCCGGAAGCTGCTGGGCCTGGAGG |
| | | ACACCGCCTTCTTCAAGGGCCTGCGGTACGGCAAGGACAACGCCGA |
| | | GGCCTCCACCCTGATGGAGATGAAGGCCTACCACGCCATCTCCCGG |
| | | GCCCTGGAGAAGGAGGGCCTGAAGGACAAGAAGTCCCCCCTGAAC |
| | | CTGTCCTCCGAGCTGCAGGACGAGATCGGCACCGCCTTCTCCCTGTT |
| | | CAAGACCGACGAGGACATCACCGGCCGGCTGAAGGACCGGGTGCA |
| | | GCCCGAGATCCTGGAGGCCCTGCTGAAGCACATCTCCTTCGACAAG |
| | | TTCGTGCAGATCTCCCTGAAGGCCCTGCGGCGGATCGTGCCCCTGA |
| | | TGGAGCAGGGCAAGCGGTACGACGAGGCCTGCGCCGAGATCTACG |
| | | GCGACCACTACGGCAAGAAGAACACCGAGGAGAAGATCTACCTGC |
| | | CCCCCATCCCCGCCGACGAGATCCGGAACCCCGTGGTGCTGCGGGC |
| | | CCTGTCCCAGGCCCGGAAGGTGATCAACGGCGTGGTGCGGCGGTAC |
| | | GGCTCCCCCGCCCGGATCCACATCGAGACCGCCCGGGAGGTGGGCA |
| | | AGTCCTTCAAGGACCGGAAGGAGATCGAGAAGCGGCAGGAGGAGA |
| | | ACCGGAAGGACCGGGAGAAGGCCGCCGCCAAGTTCCGGGAGTACT |
| | | TCCCCAACTTCGTGGGCGAGCCCAAGTCCAAGGACATCCTGAAGCT |
| | | GCGGCTGTACGAGCAGCAGCACGGCAAGTGCCTGTACTCCGGCAAG |
| | | GAGATCAACCTGGTGCGGCTGAACGAGAAGGGCTACGTGGAGATC |
| | | GACCACGCCCTGCCCTTCTCCCGGACCTGGGACGACTCCTTCAACA |
| | | ACAAGGTGCTGGTGCTGGGCTCCGAGAACCAGAACAAGGGCAACC |
| | | AGACCCCCTACGAGTACTTCAACGGCAAGGACAACTCCCGGGAGTG |
| | | GCAGGAGTTCAAGGCCCGGGTGGAGACCTCCCGGTTCCCCCGGTCC |
| | | AAGAAGCAGCGGATCCTGCTGCAGAAGTTCGACGAGGACGGCTTC |
| | | AAGGAGTGCAACCTGAACGACACCCGGTACGTGAACCGGTTCCTGT |
| | | GCCAGTTCGTGGCCGACCACATCCTGCTGACCGGCAAGGGCAAGCG |
| | | GCGGGTGTTCGCCTCCAACGGCCAGATCACCAACCTGCTGCGGGGC |
| | | TTCTGGGGCCTGCGGAAGGTGCGGGCCGAGAACGACCGGCACCAC |
| | | GCCCTGGACGCCGTGGTGGTGGCCTGCTCCACCGTGGCCATGCAGC |
| | | AGAAGATCACCCGGTTCGTGCGGTACAAGGAGATGAACGCCTTCGA |
| | | CGGCAAGACCATCGACAAGGAGACCGGCAAGGTGCTGCACCAGAA |
| | | GACCCACTTCCCCCAGCCCTGGGAGTTCTTCGCCCAGGAGGTGATG |
| | | ATCCGGGTGTTCGGCAAGCCCGACGGCAAGCCCGAGTTCGAGGAG |
| | | GCCGACACCCCCGAGAAGCTGCGGACCCTGCTGGCCGAGAAGCTGT |
| | | CCTCCCGGCCCGAGGCCGTGCACGAGTACGTGACCCCCCTGTTCGT |
| | | GTCCCGGGCCCCCAACCGGAAGATGTCCGGCGCCCACAAGGACACC |
| | | CTGCGGTCCGCCAAGCGGTTCGTGAAGCACAACGAGAAGATCTCCG |
| | | TGAAGCGGGTGTGGCTGACCGAGATCAAGCTGGCCGACCTGGAGA |
| | | ACATGGTGAACTACAAGAACGGCCGGGAGATCGAGCTGTACGAGG |
| | | CCCTGAAGGCCCGGCTGGAGGCCTACGGCGGCAACGCCAAGCAGG |
| | | CCTTCGACCCCAAGGACAACCCCTTCTACAAGAAGGGCGGCCAGCT |
| | | GGTGAAGGCCGTGCGGGTGGAGAAGACCCAGGAGTCCGGCGTGCT |
| | | GCTGAACAAGAAGAACGCCTACACCATCGCCGACAACGGCGACAT |
| | | GGTGCGGGTGGACGTGTTCTGCAAGGTGGACAAGAAGGGCAAGAA |
| | | CCAGTACTTCATCGTGCCCATCTACGCCTGGCAGGTGGCCGAGAAC |
| | | ATCCTGCCCGACATCGACTGCAAGGGCTACCGGATCGACGACTCCT |
| | | ACACCTTCTGCTTCTCCCTGCACAAGTACGACCTGATCGCCTTCCAG |
| | | AAGGACGAGAAGTCCAAGGTGGAGTTCGCCTACTACATCAACTGCG |
| | | ACTCCTCCAACGGCCGGTTCTACCTGGCCTGGCACGACAAGGGCTC |
| | | CAAGGAGCAGCAGTTCCGGATCTCCACCCAGAACCTGGTGCTGATC |
| | | CAGAAGTACCAGGTGAACGAGCTGGGCAAGGAGATCCGGCCCTGC |
| | | CGGCTGAAGAAGCGGCCCCCCGTGCGGTACCCCTACGACGTGCCCG |
| | | ACTACGCCGCCGCCCCCGCCGCCAAGAAGAAGAAGCTGGACTAG |
| 183 | Open reading | ATGGCCGCCTTCAAGCCCAACCCCATCAACTACATCCTGGGCCTGG |
| | frame for | ACATCGGCATCGCCTCCGTGGGCTGGGCCATGGTGGAGATCGACGA |
| | Nme2Cas9 | GGAGGAGAACCCCATCCGGCTGATCGACCTGGGCGTGCGGGTGTTC |
| | encoded by | GAGCGGGCCGAGGTGCCCAAGACCGGCGACTCCCTGGCCATGGCCC |
| | mRNA J | GGCGGCTGGCCCGGTCCGTGCGGCGGCTGACCCGGCGGCGGGCCCA |
| | | CCGGCTGCTGCGGGCCCGGCGGCTGCTGAAGCGGGAGGGCGTGCTG |
| | | CAGGCCGCCGACTTCGACGAGAACGGCCTGATCAAGTCCCTGCCCA |
| | | ACACCCCCTGGCAGCTGCGGGCCGCCGCCCTGGACCGGAAGCTGAC |
| | | CCCCCTGGAGTGGTCCGCCGTGCTGCTGCACCTGATCAAGCACCGG |
| | | GGCTACCTGTCCCAGCGGAAGAACGAGGGCGAGACCGCCGACAAG |
| | | GAGCTGGGCGCCCTGCTGAAGGGCGTGGCCAACAACGCCCACGCCC |
| | | TGCAGACCGGCGACTTCCGGACCCCCGCCGAGCTGGCCCTGAACAA |
| | | GTTCGAGAAGGAGTCCGGCCACATCCGGAACCAGCGGGGCGACTA |
| | | CTCCCACACCTTCTCCCGGAAGGACCTGCAGGCCGAGCTGATCCTG |
| | | CTGTTCGAGAAGCAGAAGGAGTTCGGCAACCCCCACGTGTCCGGCG |
| | | GCCTGAAGGAGGGCATCGAGACCCTGCTGATGACCCAGCGGCCCGC |
| | | CCTGTCCGGCGACGCCGTGCAGAAGATGCTGGGCCACTGCACCTTC |
| | | GAGCCCGCCGAGCCCAAGGCCGCCAAGAACACCTACACCGCCGAG |
| | | CGGTTCATCTGGCTGACCAAGCTGAACAACCTGCGGATCCTGGAGC |
| | | AGGGCTCCGAGCGGCCCCTGACCGACACCGAGCGGGCCACCCTGAT |
| | | GGACGAGCCCTACCGGAAGTCCAAGCTGACCTACGCCCAGGCCCGG |
| | | AAGCTGCTGGGCCTGGAGGACACCGCCTTCTTCAAGGGCCTGCGGT |
| | | ACGGCAAGGACAACGCCGAGGCCTCCACCCTGATGGAGATGAAGG |
| | | CCTACCACGCCATCTCCCGGGCCCTGGAGAAGGAGGGCCTGAAGGA |
| | | CAAGAAGTCCCCCCTGAACCTGTCCTCCGAGCTGCAGGACGAGATC |
| | | GGCACCGCCTTCTCCCTGTTCAAGACCGACGAGGACATCACCGGCC |
| | | GGCTGAAGGACCGGGTGCAGCCCGAGATCCTGGAGGCCCTGCTGA |
| | | AGCACATCTCCTTCGACAAGTTCGTGCAGATCTCCCTGAAGGCCCT |
| | | GCGGCGGATCGTGCCCCTGATGGAGCAGGGCAAGCGGTACGACGA |
| | | GGCCTGCGCCGAGATCTACGGCGACCACTACGGCAAGAAGAACAC |
| | | CGAGGAGAAGATCTACCTGCCCCCCATCCCCGCCGACGAGATCCGG |
| | | AACCCCGTGGTGCTGCGGGCCCTGTCCCAGGCCCGGAAGGTGATCA |
| | | ACGGCGTGGTGCGGCGGTACGGCTCCCCCGCCCGGATCCACATCGA |
| | | GACCGCCCGGGAGGTGGGCAAGTCCTTCAAGGACCGGAAGGAGAT |
| | | CGAGAAGCGGCAGGAGGAGAACCGGAAGGACCGGGAGAAGGCCG |
| | | CCGCCAAGTTCCGGGAGTACTTCCCCAACTTCGTGGGCGAGCCCAA |
| | | GTCCAAGGACATCCTGAAGCTGCGGCTGTACGAGCAGCAGCACGGC |
| | | AAGTGCCTGTACTCCGGCAAGGAGATCAACCTGGTGCGGCTGAACG |
| | | AGAAGGGCTACGTGGAGATCGACCACGCCCTGCCCTTCTCCCGGAC |
| | | CTGGGACGACTCCTTCAACAACAAGGTGCTGGTGCTGGGCTCCGAG |
| | | AACCAGAACAAGGGCAACCAGACCCCCTACGAGTACTTCAACGGC |
| | | AAGGACAACTCCCGGGAGTGGCAGGAGTTCAAGGCCCGGGTGGAG |
| | | ACCTCCCGGTTCCCCCGGTCCAAGAAGCAGCGGATCCTGCTGCAGA |
| | | AGTTCGACGAGGACGGCTTCAAGGAGTGCAACCTGAACGACACCC |
| | | GGTACGTGAACCGGTTCCTGTGCCAGTTCGTGGCCGACCACATCCT |
| | | GCTGACCGGCAAGGGCAAGCGGCGGGTGTTCGCCTCCAACGGCCA |
| | | GATCACCAACCTGCTGCGGGGCTTCTGGGGCCTGCGGAAGGTGCGG |
| | | GCCGAGAACGACCGGCACCACGCCCTGGACGCCGTGGTGGTGGCCT |
| | | GCTCCACCGTGGCCATGCAGCAGAAGATCACCCGGTTCGTGCGGTA |
| | | CAAGGAGATGAACGCCTTCGACGGCAAGACCATCGACAAGGAGAC |
| | | CGGCAAGGTGCTGCACCAGAAGACCCACTTCCCCCAGCCCTGGGAG |
| | | TTCTTCGCCCAGGAGGTGATGATCCGGGTGTTCGGCAAGCCCGACG |
| | | GCAAGCCCGAGTTCGAGGAGGCCGACACCCCCGAGAAGCTGCGGA |
| | | CCCTGCTGGCCGAGAAGCTGTCCTCCCGGCCCGAGGCCGTGCACGA |
| | | GTACGTGACCCCCCTGTTCGTGTCCCGGGCCCCCAACCGGAAGATG |
| | | TCCGGCGCCCACAAGGACACCCTGCGGTCCGCCAAGCGGTTCGTGA |
| | | AGCACAACGAGAAGATCTCCGTGAAGCGGGTGTGGCTGACCGAGA |
| | | TCAAGCTGGCCGACCTGGAGAACATGGTGAACTACAAGAACGGCC |
| | | GGGAGATCGAGCTGTACGAGGCCCTGAAGGCCCGGCTGGAGGCCT |
| | | ACGGCGGCAACGCCAAGCAGGCCTTCGACCCCAAGGACAACCCCTT |
| | | CTACAAGAAGGGCGGCCAGCTGGTGAAGGCCGTGCGGGTGGAGAA |
| | | GACCCAGGAGTCCGGCGTGCTGCTGAACAAGAAGAACGCCTACAC |
| | | CATCGCCGACAACGGCGACATGGTGCGGGTGGACGTGTTCTGCAAG |
| | | GTGGACAAGAAGGGCAAGAACCAGTACTTCATCGTGCCCATCTACG |
| | | CCTGGCAGGTGGCCGAGAACATCCTGCCCGACATCGACTGCAAGGG |
| | | CTACCGGATCGACGACTCCTACACCTTCTGCTTCTCCCTGCACAAGT |
| | | ACGACCTGATCGCCTTCCAGAAGGACGAGAAGTCCAAGGTGGAGTT |
| | | CGCCTACTACATCAACTGCGACTCCTCCAACGGCCGGTTCTACCTGG |
| | | CCTGGCACGACAAGGGCTCCAAGGAGCAGCAGTTCCGGATCTCCAC |
| | | CCAGAACCTGGTGCTGATCCAGAAGTACCAGGTGAACGAGCTGGGC |
| | | AAGGAGATCCGGCCCTGCCGGCTGAAGAAGCGGCCCCCCGTGCGG |
| 184 | Open reading | atggccgccttcaagcccaaccccatcaactacatcctgggcctggacatcggcatcgcctccgtgggctgggc |
| | frame for | catggtggagatcgacgaggaggagaaccccatccggctgatcgacctgggcgtgcgggtgttcgagcgggc |
| | Nme2Cas9 | cgaggtgcccaagaccggcgactccctggccatggcccggcggctggcccggtccgtgcggcggctgaccc |
| | encoded by | ggcggcgggcccaccggctgctgcgggcccggcggctgctgaagcgggagggcgtgctgcaggccgccg |
| | mRNA K | acttcgacgagaacggcctgatcaagtccctgcccaacaccccctggcagctgcgggccgccgccctggacc |
| | | ggaagctgacccccctggagtggtccgccgtgctgctgcacctgatcaagcaccggggctacctgtcccagcg |
| | | gaagaacgagggcgagaccgccgacaaggagctgggcgccctgctgaagggcgtggccaacaacgccca |
| | | cgccctgcagaccggcgacttccggacccccgccgagctggccctgaacaagttcgagaaggagtccggcc |
| | | acatccggaaccagcggggcgactactcccacaccttctcccggaaggacctgcaggccgagctgatcctgct |
| | | gttcgagaagcagaaggagttcggcaacccccacgtgtccggcggcctgaaggagggcatcgagaccctgct |
| | | gatgacccagcggcccgccctgtccggcgacgccgtgcagaagatgctgggccactgcaccttcgagcccgc |
| | | cgagcccaaggccgccaagaacacctacaccgccgagcggttcatctggctgaccaagctgaacaacctgcg |
| | | gatcctggagcagggctccgagcggcccctgaccgacaccgagcgggccaccctgatggacgagccctacc |
| | | ggaagtccaagctgacctacgcccaggcccggaagctgctgggcctggaggacaccgccttcttcaagggcc |
| | | tgcggtacggcaaggacaacgccgaggcctccaccctgatggagatgaaggcctaccacgccatctcccggg |
| | | ccctggagaaggagggcctgaaggacaagaagtcccccctgaacctgtcctccgagctgcaggacgagatcg |
| | | gcaccgccttctccctgttcaagaccgacgaggacatcaccggccggctgaaggaccgggtgcagcccgaga |
| | | tcctggaggccctgctgaagcacatctccttcgacaagttcgtgcagatctccctgaaggccctgcggcggatcg |
| | | tgcccctgatggagcagggcaagcggtacgacgaggcctgcgccgagatctacggcgaccactacggcaag |
| | | aagaacaccgaggagaagatctacctgccccccatccccgccgacgagatccggaaccccgtggtgctgcgg |
| | | gccctgtcccaggcccggaaggtgatcaacggcgtggtgcggcggtacggctcccccgcccggatccacatc |
| | | gagaccgcccgggaggtgggcaagtccttcaaggaccggaaggagatcgagaagcggcaggaggagaac |
| | | cggaaggaccgggagaaggccgccgccaagttccgggagtacttccccaacttcgtgggcgagcccaagtcc |
| | | aaggacatcctgaagctgcggctgtacgagcagcagcacggcaagtgcctgtactccggcaaggagatcaac |
| | | ctggtgcggctgaacgagaagggctacgtggagatcgaccacgccctgcccttctcccggacctgggacgact |
| | | ccttcaacaacaaggtgctggtgctgggctccgagaaccagaacaagggcaaccagaccccctacgagtactt |
| | | caacggcaaggacaactcccgggagtggcaggagttcaaggcccgggtggagacctcccggttcccccggt |
| | | ccaagaagcagcggatcctgctgcagaagttcgacgaggacggcttcaaggagtgcaacctgaacgacaccc |
| | | ggtacgtgaaccggttcctgtgccagttcgtggccgaccacatcctgctgaccggcaagggcaagcggcgggt |
| | | gttcgcctccaacggccagatcaccaacctgctgcggggcttctggggcctgcggaaggtgcgggccgagaa |
| | | cgaccggcaccacgccctggacgccgtggtggtggcctgctccaccgtggccatgcagcagaagatcacccg |
| | | gttcgtgcggtacaaggagatgaacgccttcgacggcaagaccatcgacaaggagaccggcaaggtgctgca |
| | | ccagaagacccacttcccccagccctgggagttcttcgcccaggaggtgatgatccgggtgttcggcaagccc |
| | | gacggcaagcccgagttcgaggaggccgacacccccgagaagctgcggaccctgctggccgagaagctgtc |
| | | ctcccggcccgaggccgtgcacgagtacgtgacccccctgttcgtgtcccgggcccccaaccggaagatgtcc |
| | | ggcgcccacaaggacaccctgcggtccgccaagcggttcgtgaagcacaacgagaagatctccgtgaagcg |
| | | ggtgtggctgaccgagatcaagctggccgacctggagaacatggtgaactacaagaacggccgggagatcga |
| | | gctgtacgaggccctgaaggcccggctggaggcctacggcggcaacgccaagcaggccttcgaccccaagg |
| | | acaaccccttctacaagaagggcggccagctggtgaaggccgtgcgggtggagaagacccaggagtccggc |
| | | gtgctgctgaacaagaagaacgcctacaccatcgccgacaacggcgacatggtgcgggtggacgtgttctgca |
| | | aggtggacaagaagggcaagaaccagtacttcatcgtgcccatctacgcctggcaggtggccgagaacatcct |
| | | gcccgacatcgactgcaagggctaccggatcgacgactcctacaccttctgcttctccctgcacaagtacgacct |
| | | gatcgccttccagaaggacgagaagtccaaggtggagttcgcctactacatcaactgcgactcctccaacggcc |
| | | ggttctacctggcctggcacgacaagggctccaaggagcagcagttccggatctccacccagaacctggtgct |
| | | gatccagaagtaccaggtgaacgagctgggcaaggagatccggccctgccggctgaagaagcggccccccg |
| | | tgcgg |
| 185 | Open reading | atgGACGGCTCCGGCGGCGGCTCCCCCAAGAAGAAGCGGAAGGTGG |
| | frame for | GCGGCTCCGGCGGCGGCgccgccttcaagcccaaccccatcaactacatcctgggcctggacat |
| | Nme2Cas9 | cggcatcgcctccgtgggctgggccatggtggagatcgacgaggaggagaaccccatccggctgatcgacct |
| | encoded by | gggcgtgcgggtgttcgagcgggccgaggtgcccaagaccggcgactccctggccatggcccggcggctgg |
| | mRNA L | cccggtccgtgcggcggctgacccggcggcgggcccaccggctgctgcgggcccggggctgctgaagcg |
| | | ggagggcgtgctgcaggccgccgacttcgacgagaacggcctgatcaagtccctgcccaacaccccctggca |
| | | gctgcgggccgccgccctggaccggaagctgacccccctggagtggtccgccgtgctgctgcacctgatcaa |
| | | gcaccggggctacctgtcccagcggaagaacgagggcgagaccgccgacaaggagctgggcgccctgctg |
| | | aagggcgtggccaacaacgcccacgccctgcagaccggcgacttccggacccccgccgagctggccctgaa |
| | | caagttcgagaaggagtccggccacatccggaaccagcggggcgactactcccacaccttctcccggaagga |
| | | cctgcaggccgagctgatcctgctgttcgagaagcagaaggagttcggcaacccccacgtgtccggcggcctg |
| | | aaggagggcatcgagaccctgctgatgacccagcggcccgccctgtccggcgacgccgtgcagaagatgct |
| | | gggccactgcaccttcgagcccgccgagcccaaggccgccaagaacacctacaccgccgagcggttcatctg |
| | | gctgaccaagctgaacaacctgcggatcctggagcagggctccgagcggcccctgaccgacaccgagcggg |
| | | ccaccctgatggacgagccctaccggaagtccaagctgacctacgcccaggcccggaagctgctgggcctgg |
| | | aggacaccgccttcttcaagggcctgcggtacggcaaggacaacgccgaggcctccaccctgatggagatga |
| | | aggcctaccacgccatctcccgggccctggagaaggagggcctgaaggacaagaagtcccccctgaacctgt |
| | | cctccgagctgcaggacgagatcggcaccgccttctccctgttcaagaccgacgaggacatcaccggccggct |
| | | gaaggaccgggtgcagcccgagatcctggaggccctgctgaagcacatctccttcgacaagttcgtgcagatct |
| | | ccctgaaggccctgcggcggatcgtgcccctgatggagcagggcaagcggtacgacgaggcctgcgccgag |
| | | atctacggcgaccactacggcaagaagaacaccgaggagaagatctacctgccccccatccccgccgacgag |
| | | atccggaaccccgtggtgctgcgggccctgtcccaggcccggaaggtgatcaacggcgtggtgcggcggtac |
| | | ggctcccccgcccggatccacatcgagaccgcccgggaggtgggcaagtccttcaaggaccggaaggagat |
| | | cgagaagcggcaggaggagaaccggaaggaccgggagaaggccgccgccaagttccgggagtacttcccc |
| | | aacttcgtgggcgagcccaagtccaaggacatcctgaagctgcggctgtacgagcagcagcacggcaagtgc |
| | | ctgtactccggcaaggagatcaacctggtgcggctgaacgagaagggctacgtggagatcgaccacgccctg |
| | | cccttctcccggacctgggacgactccttcaacaacaaggtgctggtgctgggctccgagaaccagaacaagg |
| | | gcaaccagaccccctacgagtacttcaacggcaaggacaactcccgggagtggcaggagttcaaggcccgg |
| | | gtggagacctcccggttcccccggtccaagaagcagcggatcctgctgcagaagttcgacgaggacggcttca |
| | | aggagtgcaacctgaacgacacccggtacgtgaaccggttcctgtgccagttcgtggccgaccacatcctgctg |
| | | accggcaagggcaagcggcgggtgttcgcctccaacggccagatcaccaacctgctgcggggcttctggggc |
| | | ctgcggaaggtgcgggccgagaacgaccggcaccacgccctggacgccgtggtggtggcctgctccaccgt |
| | | ggccatgcagcagaagatcacccggttcgtgcggtacaaggagatgaacgccttcgacggcaagaccatcga |
| | | caaggagaccggcaaggtgctgcaccagaagacccacttcccccagccctgggagttcttcgcccaggaggt |
| | | gatgatccgggtgttcggcaagcccgacggcaagcccgagttcgaggaggccgacacccccgagaagctgc |
| | | ggaccctgctggccgagaagctgtcctcccggcccgaggccgtgcacgagtacgtgacccccctgttcgtgtc |
| | | ccgggcccccaaccggaagatgtccggcgcccacaaggacaccctgcggtccgccaagcggttcgtgaagc |
| | | acaacgagaagatctccgtgaagcgggtgtggctgaccgagatcaagctggccgacctggagaacatggtga |
| | | actacaagaacggccgggagatcgagctgtacgaggccctgaaggcccggctggaggcctacggcggcaac |
| | | gccaagcaggccttcgaccccaaggacaaccccttctacaagaagggcggccagctggtgaaggccgtgcg |
| | | ggtggagaagacccaggagtccggcgtgctgctgaacaagaagaacgcctacaccatcgccgacaacggcg |
| | | acatggtgcgggtggacgtgttctgcaaggtggacaagaagggcaagaaccagtacttcatcgtgcccatctac |
| | | gcctggcaggtggccgagaacatcctgcccgacatcgactgcaagggctaccggatcgacgactcctacacct |
| | | tctgcttctccctgcacaagtacgacctgatcgccttccagaaggacgagaagtccaaggtggagttcgcctact |
| | | acatcaactgcgactcctccaacggccggttctacctggcctggcacgacaagggctccaaggagcagcagtt |
| | | ccggatctccacccagaacctggtgctgatccagaagtaccaggtgaacgagctgggcaaggagatccggcc |
| | | ctgccggctgaagaagcggccccccgtgcggtag |
| 186 | Open reading | ATGGACGGCTCCGGCGGCGGCTCCCCCAAGAAGAAGCGGAAGGTG |
| | frame for | GGCGGCTCCGGCGGCGGCGCCGCCTTCAAGCCCAACCCCATCAACT |
| | Nme2Cas9 with | ACATCCTGGGCCTGGACATCGGCATCGCCTCCGTGGGCTGGGCCAT |
| | HiBiT tag | GGTGGAGATCGACGAGGAGGAGAACCCCATCCGGCTGATCGACCT |
| | encoded by | GGGCGTGCGGGTGTTCGAGCGGGCCGAGGTGCCCAAGACCGGCGA |
| | mRNA M | CTCCCTGGCCATGGCCCGGCGGCTGGCCCGGTCCGTGCGGCGGCTG |
| | | ACCCGGCGGCGGGCCCACCGGCTGCTGCGGGCCCGGCGGCTGCTGA |
| | | AGCGGGAGGGCGTGCTGCAGGCCGCCGACTTCGACGAGAACGGCC |
| | | TGATCAAGTCCCTGCCCAACACCCCCTGGCAGCTGCGGGCCGCCGC |
| | | CCTGGACCGGAAGCTGACCCCCCTGGAGTGGTCCGCCGTGCTGCTG |
| | | CACCTGATCAAGCACCGGGGCTACCTGTCCCAGCGGAAGAACGAG |
| | | GGCGAGACCGCCGACAAGGAGCTGGGCGCCCTGCTGAAGGGCGTG |
| | | GCCAACAACGCCCACGCCCTGCAGACCGGCGACTTCCGGACCCCCG |
| | | CCGAGCTGGCCCTGAACAAGTTCGAGAAGGAGTCCGGCCACATCCG |
| | | GAACCAGCGGGGCGACTACTCCCACACCTTCTCCCGGAAGGACCTG |
| | | CAGGCCGAGCTGATCCTGCTGTTCGAGAAGCAGAAGGAGTTCGGCA |
| | | ACCCCCACGTGTCCGGCGGCCTGAAGGAGGGCATCGAGACCCTGCT |
| | | GATGACCCAGCGGCCCGCCCTGTCCGGCGACGCCGTGCAGAAGATG |
| | | CTGGGCCACTGCACCTTCGAGCCCGCCGAGCCCAAGGCCGCCAAGA |
| | | ACACCTACACCGCCGAGCGGTTCATCTGGCTGACCAAGCTGAACAA |
| | | CCTGCGGATCCTGGAGCAGGGCTCCGAGCGGCCCCTGACCGACACC |
| | | GAGCGGGCCACCCTGATGGACGAGCCCTACCGGAAGTCCAAGCTG |
| | | ACCTACGCCCAGGCCCGGAAGCTGCTGGGCCTGGAGGACACCGCCT |
| | | TCTTCAAGGGCCTGCGGTACGGCAAGGACAACGCCGAGGCCTCCAC |
| | | CCTGATGGAGATGAAGGCCTACCACGCCATCTCCCGGGCCCTGGAG |
| | | AAGGAGGGCCTGAAGGACAAGAAGTCCCCCCTGAACCTGTCCTCCG |
| | | AGCTGCAGGACGAGATCGGCACCGCCTTCTCCCTGTTCAAGACCGA |
| | | CGAGGACATCACCGGCCGGCTGAAGGACCGGGTGCAGCCCGAGAT |
| | | CCTGGAGGCCCTGCTGAAGCACATCTCCTTCGACAAGTTCGTGCAG |
| | | ATCTCCCTGAAGGCCCTGCGGCGGATCGTGCCCCTGATGGAGCAGG |
| | | GCAAGCGGTACGACGAGGCCTGCGCCGAGATCTACGGCGACCACT |
| | | ACGGCAAGAAGAACACCGAGGAGAAGATCTACCTGCCCCCCATCC |
| | | CCGCCGACGAGATCCGGAACCCCGTGGTGCTGCGGGCCCTGTCCCA |
| | | GGCCCGGAAGGTGATCAACGGCGTGGTGCGGCGGTACGGCTCCCCC |
| | | GCCCGGATCCACATCGAGACCGCCCGGGAGGTGGGCAAGTCCTTCA |
| | | AGGACCGGAAGGAGATCGAGAAGCGGCAGGAGGAGAACCGGAAG |
| | | GACCGGGAGAAGGCCGCCGCCAAGTTCCGGGAGTACTTCCCCAACT |
| | | TCGTGGGCGAGCCCAAGTCCAAGGACATCCTGAAGCTGCGGCTGTA |
| | | CGAGCAGCAGCACGGCAAGTGCCTGTACTCCGGCAAGGAGATCAA |
| | | CCTGGTGCGGCTGAACGAGAAGGGCTACGTGGAGATCGACCACGC |
| | | CCTGCCCTTCTCCCGGACCTGGGACGACTCCTTCAACAACAAGGTG |
| | | CTGGTGCTGGGCTCCGAGAACCAGAACAAGGGCAACCAGACCCCCT |
| | | ACGAGTACTTCAACGGCAAGGACAACTCCCGGGAGTGGCAGGAGT |
| | | TCAAGGCCCGGGTGGAGACCTCCCGGTTCCCCCGGTCCAAGAAGCA |
| | | GCGGATCCTGCTGCAGAAGTTCGACGAGGACGGCTTCAAGGAGTGC |
| | | AACCTGAACGACACCCGGTACGTGAACCGGTTCCTGTGCCAGTTCG |
| | | TGGCCGACCACATCCTGCTGACCGGCAAGGGCAAGCGGCGGGTGTT |
| | | CGCCTCCAACGGCCAGATCACCAACCTGCTGCGGGGCTTCTGGGGC |
| | | CTGCGGAAGGTGCGGGCCGAGAACGACCGGCACCACGCCCTGGAC |
| | | GCCGTGGTGGTGGCCTGCTCCACCGTGGCCATGCAGCAGAAGATCA |
| | | CCCGGTTCGTGCGGTACAAGGAGATGAACGCCTTCGACGGCAAGAC |
| | | CATCGACAAGGAGACCGGCAAGGTGCTGCACCAGAAGACCCACTT |
| | | CCCCCAGCCCTGGGAGTTCTTCGCCCAGGAGGTGATGATCCGGGTG |
| | | TTCGGCAAGCCCGACGGCAAGCCCGAGTTCGAGGAGGCCGACACC |
| | | CCCGAGAAGCTGCGGACCCTGCTGGCCGAGAAGCTGTCCTCCCGGC |
| | | CCGAGGCCGTGCACGAGTACGTGACCCCCCTGTTCGTGTCCCGGGC |
| | | CCCCAACCGGAAGATGTCCGGCGCCCACAAGGACACCCTGCGGTCC |
| | | GCCAAGCGGTTCGTGAAGCACAACGAGAAGATCTCCGTGAAGCGG |
| | | GTGTGGCTGACCGAGATCAAGCTGGCCGACCTGGAGAACATGGTGA |
| | | ACTACAAGAACGGCCGGGAGATCGAGCTGTACGAGGCCCTGAAGG |
| | | CCCGGCTGGAGGCCTACGGCGGCAACGCCAAGCAGGCCTTCGACCC |
| | | CAAGGACAACCCCTTCTACAAGAAGGGCGGCCAGCTGGTGAAGGC |
| | | CGTGCGGGTGGAGAAGACCCAGGAGTCCGGCGTGCTGCTGAACAA |
| | | GAAGAACGCCTACACCATCGCCGACAACGGCGACATGGTGCGGGT |
| | | GGACGTGTTCTGCAAGGTGGACAAGAAGGGCAAGAACCAGTACTT |
| | | CATCGTGCCCATCTACGCCTGGCAGGTGGCCGAGAACATCCTGCCC |
| | | GACATCGACTGCAAGGGCTACCGGATCGACGACTCCTACACCTTCT |
| | | GCTTCTCCCTGCACAAGTACGACCTGATCGCCTTCCAGAAGGACGA |
| | | GAAGTCCAAGGTGGAGTTCGCCTACTACATCAACTGCGACTCCTCC |
| | | AACGGCCGGTTCTACCTGGCCTGGCACGACAAGGGCTCCAAGGAGC |
| | | AGCAGTTCCGGATCTCCACCCAGAACCTGGTGCTGATCCAGAAGTA |
| | | CCAGGTGAACGAGCTGGGCAAGGAGATCCGGCCCTGCCGGCTGAA |
| | | GAAGCGGCCCCCCGTGCGGTCCGAGTCCGCCACCCCCGAGTCCGTG |
| | | TCCGGCTGGCGGCTGTTCAAGAAGATCTCCTAG |
| 187 | Open reading | atgGACGGCTCCGGCGGCGGCTCCCCCAAGAAGAAGCGGAAGGTGG |
| | frame for | GCGGCTCCGGCGGCGGCgccgccttcaagcccaaccccatcaactacatcctgggcctggacat |
| | Nme2Cas9 | cggcatcgcctccgtgggctgggccatggtggagatcgacgaggaggagaaccccatccggctgatcgacct |
| | encoded by | gggcgtgcgggtgttcgagcgggccgaggtgcccaagaccggcgactccctggccatggcccggcggctgg |
| | mRNA N | cccggtccgtgcggggctgacccggcggcgggcccaccggctgctgcgggcccggcggctgctgaagcg |
| | | ggagggcgtgctgcaggccgccgacttcgacgagaacggcctgatcaagtccctgcccaacaccccctggca |
| | | gctgcgggccgccgccctggaccggaagctgacccccctggagtggtccgccgtgctgctgcacctgatcaa |
| | | gcaccggggctacctgtcccagcggaagaacgagggcgagaccgccgacaaggagctgggcgccctgctg |
| | | aagggcgtggccaacaacgcccacgccctgcagaccggcgacttccggacccccgccgagctggccctgaa |
| | | caagttcgagaaggagtccggccacatccggaaccagcggggcgactactcccacaccttctcccggaagga |
| | | cctgcaggccgagctgatcctgctgttcgagaagcagaaggagttcggcaacccccacgtgtccggcggcctg |
| | | aaggagggcatcgagaccctgctgatgacccagcggcccgccctgtccggcgacgccgtgcagaagatgct |
| | | gggccactgcaccttcgagcccgccgagcccaaggccgccaagaacacctacaccgccgagcggttcatctg |
| | | gctgaccaagctgaacaacctgcggatcctggagcagggctccgagcggcccctgaccgacaccgagcggg |
| | | ccaccctgatggacgagccctaccggaagtccaagctgacctacgcccaggcccggaagctgctgggcctgg |
| | | aggacaccgccttcttcaagggcctgcggtacggcaaggacaacgccgaggcctccaccctgatggagatga |
| | | aggcctaccacgccatctcccgggccctggagaaggagggcctgaaggacaagaagtcccccctgaacctgt |
| | | cctccgagctgcaggacgagatcggcaccgccttctccctgttcaagaccgacgaggacatcaccggccggct |
| | | gaaggaccgggtgcagcccgagatcctggaggccctgctgaagcacatctccttcgacaagttcgtgcagatct |
| | | ccctgaaggccctgcggcggatcgtgcccctgatggagcagggcaagcggtacgacgaggcctgcgccgag |
| | | atctacggcgaccactacggcaagaagaacaccgaggagaagatctacctgccccccatccccgccgacgag |
| | | atccggaaccccgtggtgctgcgggccctgtcccaggcccggaaggtgatcaacggcgtggtgcggggtac |
| | | ggctcccccgcccggatccacatcgagaccgcccgggaggtgggcaagtccttcaaggaccggaaggagat |
| | | cgagaagcggcaggaggagaaccggaaggaccgggagaaggccgccgccaagttccgggagtacttcccc |
| | | aacttcgtgggcgagcccaagtccaaggacatcctgaagctgcggctgtacgagcagcagcacggcaagtgc |
| | | ctgtactccggcaaggagatcaacctggtgcggctgaacgagaagggctacgtggagatcgaccacgccctg |
| | | cccttctcccggacctgggacgactccttcaacaacaaggtgctggtgctgggctccgagaaccagaacaagg |
| | | gcaaccagaccccctacgagtacttcaacggcaaggacaactcccgggagtggcaggagttcaaggcccgg |
| | | gtggagacctcccggttcccccggtccaagaagcagcggatcctgctgcagaagttcgacgaggacggcttca |
| | | aggagtgcaacctgaacgacacccggtacgtgaaccggttcctgtgccagttcgtggccgaccacatcctgctg |
| | | accggcaagggcaagcggcgggtgttcgcctccaacggccagatcaccaacctgctgcggggcttctggggc |
| | | ctgcggaaggtgcgggccgagaacgaccggcaccacgccctggacgccgtggtggtggcctgctccaccgt |
| | | ggccatgcagcagaagatcacccggttcgtgcggtacaaggagatgaacgccttcgacggcaagaccatcga |
| | | caaggagaccggcaaggtgctgcaccagaagacccacttcccccagccctgggagttcttcgcccaggaggt |
| | | gatgatccgggtgttcggcaagcccgacggcaagcccgagttcgaggaggccgacacccccgagaagctgc |
| | | ggaccctgctggccgagaagctgtcctcccggcccgaggccgtgcacgagtacgtgacccccctgttcgtgtc |
| | | ccgggcccccaaccggaagatgtccggcgcccacaaggacaccctgcggtccgccaagcggttcgtgaagc |
| | | acaacgagaagatctccgtgaagcgggtgtggctgaccgagatcaagctggccgacctggagaacatggtga |
| | | actacaagaacggccgggagatcgagctgtacgaggccctgaaggcccggctggaggcctacggcggcaac |
| | | gccaagcaggccttcgaccccaaggacaaccccttctacaagaagggcggccagctggtgaaggccgtgcg |
| | | ggtggagaagacccaggagtccggcgtgctgctgaacaagaagaacgcctacaccatcgccgacaacggcg |
| | | acatggtgcgggtggacgtgttctgcaaggtggacaagaagggcaagaaccagtacttcatcgtgcccatctac |
| | | gcctggcaggtggccgagaacatcctgcccgacatcgactgcaagggctaccggatcgacgactcctacacct |
| | | tctgcttctccctgcacaagtacgacctgatcgccttccagaaggacgagaagtccaaggtggagttcgcctact |
| | | acatcaactgcgactcctccaacggccggttctacctggcctggcacgacaagggctccaaggagcagcagtt |
| | | ccggatctccacccagaacctggtgctgatccagaagtaccaggtgaacgagctgggcaaggagatccggcc |
| | | ctgccggctgaagaagcggccccccgtgcggTCCGGAAAGCGGACCGCCGACGGCT |
| | | CCGGAGGAGGAAGCCCCGCCGCCAAGAAGAAGAAGCTGGACtag |
| 188 | Open reading | atgGACGGCTCCGGCGGCGGCTCCCCCAAGAAGAAGCGGAAGGTGG |
| | frame for | AGGACAAGCGGCCCGCCGCCACCAAGAAGGCCGGCCAGGCCAAGA |
| | Nme2Cas9 | AGAAGAAGGGCGGCTCCGGCGGCGGCgccgccttcaagcccaaccccatcaactacat |
| | encoded by | cctgggcctggacatcggcatcgcctccgtgggctgggccatggtggagatcgacgaggaggagaaccccat |
| | mRNA O | ccggctgatcgacctgggcgtgcgggtgttcgagcgggccgaggtgcccaagaccggcgactccctggccat |
| | | ggcccggggctggcccggtccgtgcggcggctgacccggcgggggcccaccggctgctgcgggcccg |
| | | gcggctgctgaagcgggagggcgtgctgcaggccgccgacttcgacgagaacggcctgatcaagtccctgcc |
| | | caacaccccctggcagctgcgggccgccgccctggaccggaagctgacccccctggagtggtccgccgtgct |
| | | gctgcacctgatcaagcaccggggctacctgtcccagcggaagaacgagggcgagaccgccgacaaggag |
| | | ctgggcgccctgctgaagggcgtggccaacaacgcccacgccctgcagaccggcgacttccggacccccgc |
| | | cgagctggccctgaacaagttcgagaaggagtccggccacatccggaaccagcggggcgactactcccacac |
| | | cttctcccggaaggacctgcaggccgagctgatcctgctgttcgagaagcagaaggagttcggcaacccccac |
| | | gtgtccggcggcctgaaggagggcatcgagaccctgctgatgacccagcggcccgccctgtccggcgacgc |
| | | cgtgcagaagatgctgggccactgcaccttcgagcccgccgagcccaaggccgccaagaacacctacaccg |
| | | ccgagcggttcatctggctgaccaagctgaacaacctgcggatcctggagcagggctccgagcggcccctga |
| | | ccgacaccgagcgggccaccctgatggacgagccctaccggaagtccaagctgacctacgcccaggcccgg |
| | | aagctgctgggcctggaggacaccgccttcttcaagggcctgcggtacggcaaggacaacgccgaggcctcc |
| | | accctgatggagatgaaggcctaccacgccatctcccgggccctggagaaggagggcctgaaggacaagaa |
| | | gtcccccctgaacctgtcctccgagctgcaggacgagatcggcaccgccttctccctgttcaagaccgacgagg |
| | | acatcaccggccggctgaaggaccgggtgcagcccgagatcctggaggccctgctgaagcacatctccttcga |
| | | caagttcgtgcagatctccctgaaggccctgcggcggatcgtgcccctgatggagcagggcaagcggtacgac |
| | | gaggcctgcgccgagatctacggcgaccactacggcaagaagaacaccgaggagaagatctacctgccccc |
| | | catccccgccgacgagatccggaaccccgtggtgctgcgggccctgtcccaggcccggaaggtgatcaacgg |
| | | cgtggtgcggcggtacggctcccccgcccggatccacatcgagaccgcccgggaggtgggcaagtccttcaa |
| | | ggaccggaaggagatcgagaagcggcaggaggagaaccggaaggaccgggagaaggccgccgccaagtt |
| | | ccgggagtacttccccaacttcgtgggcgagcccaagtccaaggacatcctgaagctgcggctgtacgagcag |
| | | cagcacggcaagtgcctgtactccggcaaggagatcaacctggtgcggctgaacgagaagggctacgtggag |
| | | atcgaccacgccctgcccttctcccggacctgggacgactccttcaacaacaaggtgctggtgctgggctccga |
| | | gaaccagaacaagggcaaccagaccccctacgagtacttcaacggcaaggacaactcccgggagtggcagg |
| | | agttcaaggcccgggtggagacctcccggttcccccggtccaagaagcagcggatcctgctgcagaagttcga |
| | | cgaggacggcttcaaggagtgcaacctgaacgacacccggtacgtgaaccggttcctgtgccagttcgtggcc |
| | | gaccacatcctgctgaccggcaagggcaagcggcgggtgttcgcctccaacggccagatcaccaacctgctg |
| | | cggggcttctggggcctgcggaaggtgcgggccgagaacgaccggcaccacgccctggacgccgtggtggt |
| | | ggcctgctccaccgtggccatgcagcagaagatcacccggttcgtgcggtacaaggagatgaacgccttcgac |
| | | ggcaagaccatcgacaaggagaccggcaaggtgctgcaccagaagacccacttcccccagccctgggagttc |
| | | ttcgcccaggaggtgatgatccgggtgttcggcaagcccgacggcaagcccgagttcgaggaggccgacacc |
| | | cccgagaagctgcggaccctgctggccgagaagctgtcctcccggcccgaggccgtgcacgagtacgtgacc |
| | | cccctgttcgtgtcccgggcccccaaccggaagatgtccggcgcccacaaggacaccctgcggtccgccaag |
| | | cggttcgtgaagcacaacgagaagatctccgtgaagcgggtgtggctgaccgagatcaagctggccgacctg |
| | | gagaacatggtgaactacaagaacggccgggagatcgagctgtacgaggccctgaaggcccggctggaggc |
| | | ctacggcggcaacgccaagcaggccttcgaccccaaggacaaccccttctacaagaagggggccagctggt |
| | | gaaggccgtgcgggtggagaagacccaggagtccggcgtgctgctgaacaagaagaacgcctacaccatcg |
| | | ccgacaacggcgacatggtgcgggtggacgtgttctgcaaggtggacaagaagggcaagaaccagtacttcat |
| | | cgtgcccatctacgcctggcaggtggccgagaacatcctgcccgacatcgactgcaagggctaccggatcgac |
| | | gactcctacaccttctgcttctccctgcacaagtacgacctgatcgccttccagaaggacgagaagtccaaggtg |
| | | gagttcgcctactacatcaactgcgactcctccaacggccggttctacctggcctggcacgacaagggctccaa |
| | | ggagcagcagttccggatctccacccagaacctggtgctgatccagaagtaccaggtgaacgagctgggcaa |
| | | ggagatccggccctgccggctgaagaagcggccccccgtgcggtag |
| 189 | Open reading | atgGACGGCTCCGGCGGCGGCTCCCCCAAGAAGAAGCGGAAGGTGG |
| | frame for | AGGACAAGCGGCCCGCCGCCACCAAGAAGGCCGGCCAGGCCAAGA |
| | Nme2Cas9 with | AGAAGAAGGGCGGCTCCGGCGGCGGCGCCGCCTTCAAGCCCAACC |
| | HiBiT tag | CCATCAACTACATCCTGGGCCTGGACATCGGCATCGCCTCCGTGGG |
| | encoded by | CTGGGCCATGGTGGAGATCGACGAGGAGGAGAACCCCATCCGGCT |
| | mRNA P | GATCGACCTGGGCGTGCGGGTGTTCGAGCGGGCCGAGGTGCCCAAG |
| | | ACCGGCGACTCCCTGGCCATGGCCCGGCGGCTGGCCCGGTCCGTGC |
| | | GGCGGCTGACCCGGCGGCGGGCCCACCGGCTGCTGCGGGCCCGGC |
| | | GGCTGCTGAAGCGGGAGGGCGTGCTGCAGGCCGCCGACTTCGACG |
| | | AGAACGGCCTGATCAAGTCCCTGCCCAACACCCCCTGGCAGCTGCG |
| | | GGCCGCCGCCCTGGACCGGAAGCTGACCCCCCTGGAGTGGTCCGCC |
| | | GTGCTGCTGCACCTGATCAAGCACCGGGGCTACCTGTCCCAGCGGA |
| | | AGAACGAGGGCGAGACCGCCGACAAGGAGCTGGGCGCCCTGCTGA |
| | | AGGGCGTGGCCAACAACGCCCACGCCCTGCAGACCGGCGACTTCCG |
| | | GACCCCCGCCGAGCTGGCCCTGAACAAGTTCGAGAAGGAGTCCGGC |
| | | CACATCCGGAACCAGCGGGGCGACTACTCCCACACCTTCTCCCGGA |
| | | AGGACCTGCAGGCCGAGCTGATCCTGCTGTTCGAGAAGCAGAAGG |
| | | AGTTCGGCAACCCCCACGTGTCCGGCGGCCTGAAGGAGGGCATCGA |
| | | GACCCTGCTGATGACCCAGCGGCCCGCCCTGTCCGGCGACGCCGTG |
| | | CAGAAGATGCTGGGCCACTGCACCTTCGAGCCCGCCGAGCCCAAGG |
| | | CCGCCAAGAACACCTACACCGCCGAGCGGTTCATCTGGCTGACCAA |
| | | GCTGAACAACCTGCGGATCCTGGAGCAGGGCTCCGAGCGGCCCCTG |
| | | ACCGACACCGAGCGGGCCACCCTGATGGACGAGCCCTACCGGAAG |
| | | TCCAAGCTGACCTACGCCCAGGCCCGGAAGCTGCTGGGCCTGGAGG |
| | | ACACCGCCTTCTTCAAGGGCCTGCGGTACGGCAAGGACAACGCCGA |
| | | GGCCTCCACCCTGATGGAGATGAAGGCCTACCACGCCATCTCCCGG |
| | | GCCCTGGAGAAGGAGGGCCTGAAGGACAAGAAGTCCCCCCTGAAC |
| | | CTGTCCTCCGAGCTGCAGGACGAGATCGGCACCGCCTTCTCCCTGTT |
| | | CAAGACCGACGAGGACATCACCGGCCGGCTGAAGGACCGGGTGCA |
| | | GCCCGAGATCCTGGAGGCCCTGCTGAAGCACATCTCCTTCGACAAG |
| | | TTCGTGCAGATCTCCCTGAAGGCCCTGCGGCGGATCGTGCCCCTGA |
| | | TGGAGCAGGGCAAGCGGTACGACGAGGCCTGCGCCGAGATCTACG |
| | | GCGACCACTACGGCAAGAAGAACACCGAGGAGAAGATCTACCTGC |
| | | CCCCCATCCCCGCCGACGAGATCCGGAACCCCGTGGTGCTGCGGGC |
| | | CCTGTCCCAGGCCCGGAAGGTGATCAACGGCGTGGTGCGGCGGTAC |
| | | GGCTCCCCCGCCCGGATCCACATCGAGACCGCCCGGGAGGTGGGCA |
| | | AGTCCTTCAAGGACCGGAAGGAGATCGAGAAGCGGCAGGAGGAGA |
| | | ACCGGAAGGACCGGGAGAAGGCCGCCGCCAAGTTCCGGGAGTACT |
| | | TCCCCAACTTCGTGGGCGAGCCCAAGTCCAAGGACATCCTGAAGCT |
| | | GCGGCTGTACGAGCAGCAGCACGGCAAGTGCCTGTACTCCGGCAAG |
| | | GAGATCAACCTGGTGCGGCTGAACGAGAAGGGCTACGTGGAGATC |
| | | GACCACGCCCTGCCCTTCTCCCGGACCTGGGACGACTCCTTCAACA |
| | | ACAAGGTGCTGGTGCTGGGCTCCGAGAACCAGAACAAGGGCAACC |
| | | AGACCCCCTACGAGTACTTCAACGGCAAGGACAACTCCCGGGAGTG |
| | | GCAGGAGTTCAAGGCCCGGGTGGAGACCTCCCGGTTCCCCCGGTCC |
| | | AAGAAGCAGCGGATCCTGCTGCAGAAGTTCGACGAGGACGGCTTC |
| | | AAGGAGTGCAACCTGAACGACACCCGGTACGTGAACCGGTTCCTGT |
| | | GCCAGTTCGTGGCCGACCACATCCTGCTGACCGGCAAGGGCAAGCG |
| | | GCGGGTGTTCGCCTCCAACGGCCAGATCACCAACCTGCTGCGGGGC |
| | | TTCTGGGGCCTGCGGAAGGTGCGGGCCGAGAACGACCGGCACCAC |
| | | GCCCTGGACGCCGTGGTGGTGGCCTGCTCCACCGTGGCCATGCAGC |
| | | AGAAGATCACCCGGTTCGTGCGGTACAAGGAGATGAACGCCTTCGA |
| | | CGGCAAGACCATCGACAAGGAGACCGGCAAGGTGCTGCACCAGAA |
| | | GACCCACTTCCCCCAGCCCTGGGAGTTCTTCGCCCAGGAGGTGATG |
| | | ATCCGGGTGTTCGGCAAGCCCGACGGCAAGCCCGAGTTCGAGGAG |
| | | GCCGACACCCCCGAGAAGCTGCGGACCCTGCTGGCCGAGAAGCTGT |
| | | CCTCCCGGCCCGAGGCCGTGCACGAGTACGTGACCCCCCTGTTCGT |
| | | GTCCCGGGCCCCCAACCGGAAGATGTCCGGCGCCCACAAGGACACC |
| | | CTGCGGTCCGCCAAGCGGTTCGTGAAGCACAACGAGAAGATCTCCG |
| | | TGAAGCGGGTGTGGCTGACCGAGATCAAGCTGGCCGACCTGGAGA |
| | | ACATGGTGAACTACAAGAACGGCCGGGAGATCGAGCTGTACGAGG |
| | | CCCTGAAGGCCCGGCTGGAGGCCTACGGCGGCAACGCCAAGCAGG |
| | | CCTTCGACCCCAAGGACAACCCCTTCTACAAGAAGGGCGGCCAGCT |
| | | GGTGAAGGCCGTGCGGGTGGAGAAGACCCAGGAGTCCGGCGTGCT |
| | | GCTGAACAAGAAGAACGCCTACACCATCGCCGACAACGGCGACAT |
| | | GGTGCGGGTGGACGTGTTCTGCAAGGTGGACAAGAAGGGCAAGAA |
| | | CCAGTACTTCATCGTGCCCATCTACGCCTGGCAGGTGGCCGAGAAC |
| | | ATCCTGCCCGACATCGACTGCAAGGGCTACCGGATCGACGACTCCT |
| | | ACACCTTCTGCTTCTCCCTGCACAAGTACGACCTGATCGCCTTCCAG |
| | | AAGGACGAGAAGTCCAAGGTGGAGTTCGCCTACTACATCAACTGCG |
| | | ACTCCTCCAACGGCCGGTTCTACCTGGCCTGGCACGACAAGGGCTC |
| | | CAAGGAGCAGCAGTTCCGGATCTCCACCCAGAACCTGGTGCTGATC |
| | | CAGAAGTACCAGGTGAACGAGCTGGGCAAGGAGATCCGGCCCTGC |
| | | CGGCTGAAGAAGCGGCCCCCCGTGCGGTCCGAGTCCGCCACCCCCG |
| | | AGTCCGTGTCCGGCTGGCGGCTGTTCAAGAAGATCTCCTAG |
| 190 | Open reading | atgGACGGCTCCGGCGGCGGCTCCGAGGACAAGCGGCCCGCCGCCAC |
| | frame for | CAAGAAGGCCGGCCAGGCCAAGAAGAAGAAGGGCGGCTCCGGCGG |
| | Nme2Cas9 | CGGCgccgccttcaagcccaaccccatcaactacatcctgggcctggacatcggcatcgcctccgtgggct |
| | encoded by | gggccatggtggagatcgacgaggaggagaaccccatccggctgatcgacctgggcgtgcgggtgttcgagc |
| | mRNA Q | gggccgaggtgcccaagaccggcgactccctggccatggcccggcggctggcccggtccgtgcggcggct |
| | | gacccggcgggggcccaccggctgctgcgggcccggcggctgctgaagcgggagggcgtgctgcaggc |
| | | cgccgacttcgacgagaacggcctgatcaagtccctgcccaacaccccctggcagctgcgggccgccgccct |
| | | ggaccggaagctgacccccctggagtggtccgccgtgctgctgcacctgatcaagcaccggggctacctgtcc |
| | | cagcggaagaacgagggcgagaccgccgacaaggagctgggcgccctgctgaagggcgtggccaacaac |
| | | gcccacgccctgcagaccggcgacttccggacccccgccgagctggccctgaacaagttcgagaaggagtcc |
| | | ggccacatccggaaccagcggggcgactactcccacaccttctcccggaaggacctgcaggccgagctgatc |
| | | ctgctgttcgagaagcagaaggagttcggcaacccccacgtgtccggcggcctgaaggagggcatcgagacc |
| | | ctgctgatgacccagcggcccgccctgtccggcgacgccgtgcagaagatgctgggccactgcaccttcgag |
| | | cccgccgagcccaaggccgccaagaacacctacaccgccgagcggttcatctggctgaccaagctgaacaac |
| | | ctgcggatcctggagcagggctccgagcggcccctgaccgacaccgagcgggccaccctgatggacgagcc |
| | | ctaccggaagtccaagctgacctacgcccaggcccggaagctgctgggcctggaggacaccgccttcttcaag |
| | | ggcctgcggtacggcaaggacaacgccgaggcctccaccctgatggagatgaaggcctaccacgccatctcc |
| | | cgggccctggagaaggagggcctgaaggacaagaagtcccccctgaacctgtcctccgagctgcaggacga |
| | | gatcggcaccgccttctccctgttcaagaccgacgaggacatcaccggccggctgaaggaccgggtgcagcc |
| | | cgagatcctggaggccctgctgaagcacatctccttcgacaagttcgtgcagatctccctgaaggccctgcggc |
| | | ggatcgtgcccctgatggagcagggcaagcggtacgacgaggcctgcgccgagatctacggcgaccactac |
| | | ggcaagaagaacaccgaggagaagatctacctgccccccatccccgccgacgagatccggaaccccgtggt |
| | | gctgcgggccctgtcccaggcccggaaggtgatcaacggcgtggtgcggcggtacggctcccccgcccgga |
| | | tccacatcgagaccgcccgggaggtgggcaagtccttcaaggaccggaaggagatcgagaagcggcagga |
| | | ggagaaccggaaggaccgggagaaggccgccgccaagttccgggagtacttccccaacttcgtgggcgagc |
| | | ccaagtccaaggacatcctgaagctgcggctgtacgagcagcagcacggcaagtgcctgtactccggcaagg |
| | | agatcaacctggtgcggctgaacgagaagggctacgtggagatcgaccacgccctgcccttctcccggacctg |
| | | ggacgactccttcaacaacaaggtgctggtgctgggctccgagaaccagaacaagggcaaccagacccccta |
| | | cgagtacttcaacggcaaggacaactcccgggagtggcaggagttcaaggcccgggtggagacctcccggtt |
| | | cccccggtccaagaagcagcggatcctgctgcagaagttcgacgaggacggcttcaaggagtgcaacctgaa |
| | | cgacacccggtacgtgaaccggttcctgtgccagttcgtggccgaccacatcctgctgaccggcaagggcaag |
| | | cggcgggtgttcgcctccaacggccagatcaccaacctgctgcggggcttctggggcctgcggaaggtgcgg |
| | | gccgagaacgaccggcaccacgccctggacgccgtggtggtggcctgctccaccgtggccatgcagcagaa |
| | | gatcacccggttcgtgcggtacaaggagatgaacgccttcgacggcaagaccatcgacaaggagaccggcaa |
| | | ggtgctgcaccagaagacccacttcccccagccctgggagttcttcgcccaggaggtgatgatccgggtgttcg |
| | | gcaagcccgacggcaagcccgagttcgaggaggccgacacccccgagaagctgcggaccctgctggccga |
| | | gaagctgtcctcccggcccgaggccgtgcacgagtacgtgacccccctgttcgtgtcccgggcccccaaccgg |
| | | aagatgtccggcgcccacaaggacaccctgcggtccgccaagcggttcgtgaagcacaacgagaagatctcc |
| | | gtgaagcgggtgtggctgaccgagatcaagctggccgacctggagaacatggtgaactacaagaacggccgg |
| | | gagatcgagctgtacgaggccctgaaggcccggctggaggcctacggcggcaacgccaagcaggccttcga |
| | | ccccaaggacaaccccttctacaagaagggcggccagctggtgaaggccgtgcgggtggagaagacccagg |
| | | agtccggcgtgctgctgaacaagaagaacgcctacaccatcgccgacaacggcgacatggtgcgggtggacg |
| | | tgttctgcaaggtggacaagaagggcaagaaccagtacttcatcgtgcccatctacgcctggcaggtggccgag |
| | | aacatcctgcccgacatcgactgcaagggctaccggatcgacgactcctacaccttctgcttctccctgcacaag |
| | | tacgacctgatcgccttccagaaggacgagaagtccaaggtggagttcgcctactacatcaactgcgactcctcc |
| | | aacggccggttctacctggcctggcacgacaagggctccaaggagcagcagttccggatctccacccagaac |
| | | ctggtgctgatccagaagtaccaggtgaacgagctgggcaaggagatccggccctgccggctgaagaagcgg |
| | | ccccccgtgcggtag |
| 191 | Exemplary amino | MAAFKPNSINYILGLDIGIASVGWAMVEIDEEENPIRLIDLGVRVFERAE |
| | acid sequence of | VPKTGDSLAMARRLARSVRRLTRRRAHRLLRTRRLLKREGVLQAANF |
| | Nme1Cas9 | DENGLIKSLPNTPWQLRAAALDRKLTPLEWSAVLLHLIKHRGYLSQRK |
| | cleavase | NEGETADKELGALLKGVAGNAHALQTGDFRTPAELALNKFEKESGHI |
| | | RNQRSDYSHTFSRKDLQAELILLFEKQKEFGNPHVSGGLKEGIETLLMT |
| | | QRPALSGDAVQKMLGHCTFEPAEPKAAKNTYTAERFIWLTKLNNLRIL |
| | | EQGSERPLTDTERATLMDEPYRKSKLTYAQARKLLGLEDTAFFKGLRY |
| | | GKDNAEASTLMEMKAYHAISRALEKEGLKDKKSPLNLSPELQDEIGTA |
| | | FSLFKTDEDITGRLKDRIQPEILEALLKHISFDKFVQISLKALRRIVPLME |
| | | QGKRYDEACAEIYGDHYGKKNTEEKIYLPPIPADEIRNPVVLRALSQAR |
| | | KVINGVVRRYGSPARIHIETAREVGKSFKDRKEIEKRQEENRKDREKA |
| | | AAKFREYFPNFVGEPKSKDILKLRLYEQQHGKCLYSGKEINLGRLNEK |
| | | GYVEIDHALPFSRTWDDSFNNKVLVLGSENQNKGNQTPYEYFNGKDN |
| | | SREWQEFKARVETSRFPRSKKQRILLQKFDEDGFKERNLNDTRYVNRF |
| | | LCQFVADRMRLTGKGKKRVFASNGQITNLLRGFWGLRKVRAENDRH |
| | | HALDAVVVACSTVAMQQKITRFVRYKEMNAFDGKTIDKETGEVLHQ |
| | | KTHFPQPWEFFAQEVMIRVFGKPDGKPEFEEADTLEKLRTLLAEKLSSR |
| | | PEAVHEYVTPLFVSRAPNRKMSGQGHMETVKSAKRLDEGVSVLRVPL |
| | | TQLKLKDLEKMVNREREPKLYEALKARLEAHKDDPAKAFAEPFYKYD |
| | | KAGNRTQQVKAVRVEQVQKTGVWVRNHNGIADNATMVRVDVFEKG |
| | | DKYYLVPIYSWQVAKGILPDRAVVQGKDEEDWQLIDDSFNFKFSLHPN |
| | | DLVEVITKKARMFGYFASCHRGTGNINIRIHDLDHKIGKNGILEGIGVK |
| | | TALSFQKYQIDELGKEIRPCRLKKRPPVR |
| 192 | Exemplary | GCTGCTTTTAAGCCTAATTCTATTAATTATATTCTTGGTCTTGATATT |
| | coding sequence | GGTATTGCTTCTGTTGGTTGGGCTATGGTTGAGATTGATGAGGAGG |
| | encoding | AGAATCCTATTCGTCTTATTGATCTTGGTGTTCGTGTTTTTGAGCGT |
| | Nme1Cas9 | GCTGAGGTTCCTAAGACTGGTGATTCTCTTGCTATGGCTCGTCGTCT |
| | cleavase | TGCTCGTTCTGTTCGTCGTCTTACTCGTCGTCGTGCTCATCGTCTTCT |
| | | TCGTACTCGTCGTCTTCTTAAGCGTGAGGGTGTTCTTCAGGCTGCTA |
| | | ATTTTGATGAGAATGGTCTTATTAAGTCTCTTCCTAATACTCCTTGG |
| | | CAGCTTCGTGCTGCTGCTCTTGATCGTAAGCTTACTCCTCTTGAGTG |
| | | GTCTGCTGTTCTTCTTCATCTTATTAAGCATCGTGGTTATCTTTCTCA |
| | | GCGTAAGAATGAGGGTGAGACTGCTGATAAGGAGCTTGGTGCTCTT |
| | | CTTAAGGGTGTTGCTGGTAATGCTCATGCTCTTCAGACTGGTGATTT |
| | | TCGTACTCCTGCTGAGCTTGCTCTTAATAAGTTTGAGAAGGAGTCTG |
| | | GTCATATTCGTAATCAGCGTTCTGATTATTCTCATACTTTTTCTCGTA |
| | | AGGATCTTCAGGCTGAGCTTATTCTTCTTTTTGAGAAGCAGAAGGA |
| | | GTTTGGTAATCCTCATGTTTCTGGTGGTCTTAAGGAGGGTATTGAGA |
| | | CTCTTCTTATGACTCAGCGTCCTGCTCTTTCTGGTGATGCTGTTCAG |
| | | AAGATGCTTGGTCATTGTACTTTTGAGCCTGCTGAGCCTAAGGCTGC |
| | | TAAGAATACTTATACTGCTGAGCGTTTTATTTGGCTTACTAAGCTTA |
| | | ATAATCTTCGTATTCTTGAGCAGGGTTCTGAGCGTCCTCTTACTGAT |
| | | ACTGAGCGTGCTACTCTTATGGATGAGCCTTATCGTAAGTCTAAGCT |
| | | TACTTATGCTCAGGCTCGTAAGCTTCTTGGTCTTGAGGATACTGCTT |
| | | TTTTTAAGGGTCTTCGTTATGGTAAGGATAATGCTGAGGCTTCTACT |
| | | CTTATGGAGATGAAGGCTTATCATGCTATTTCTCGTGCTCTTGAGAA |
| | | GGAGGGTCTTAAGGATAAGAAGTCTCCTCTTAATCTTTCTCCTGAGC |
| | | TTCAGGATGAGATTGGTACTGCTTTTTCTCTTTTTAAGACTGATGAG |
| | | GATATTACTGGTCGTCTTAAGGATCGTATTCAGCCTGAGATTCTTGA |
| | | GGCTCTTCTTAAGCATATTTCTTTTGATAAGTTTGTTCAGATTTCTCT |
| | | TAAGGCTCTTCGTCGTATTGTTCCTCTTATGGAGCAGGGTAAGCGTT |
| | | ATGATGAGGCTTGTGCTGAGATTTATGGTGATCATTATGGTAAGAA |
| | | GAATACTGAGGAGAAGATTTATCTTCCTCCTATTCCTGCTGATGAGA |
| | | TTCGTAATCCTGTTGTTCTTCGTGCTCTTTCTCAGGCTCGTAAGGTTA |
| | | TTAATGGTGTTGTTCGTCGTTATGGTTCTCCTGCTCGTATTCATATTG |
| | | AGACTGCTCGTGAGGTTGGTAAGTCTTTTAAGGATCGTAAGGAGAT |
| | | TGAGAAGCGTCAGGAGGAGAATCGTAAGGATCGTGAGAAGGCTGC |
| | | TGCTAAGTTTCGTGAGTATTTTCCTAATTTTGTTGGTGAGCCTAAGT |
| | | CTAAGGATATTCTTAAGCTTCGTCTTTATGAGCAGCAGCATGGTAA |
| | | GTGTCTTTATTCTGGTAAGGAGATTAATCTTGGTCGTCTTAATGAGA |
| | | AGGGTTATGTTGAGATTGATCATGCTCTTCCTTTTTCTCGTACTTGG |
| | | GATGATTCTTTTAATAATAAGGTTCTTGTTCTTGGTTCTGAGAATCA |
| | | GAATAAGGGTAATCAGACTCCTTATGAGTATTTTAATGGTAAGGAT |
| | | AATTCTCGTGAGTGGCAGGAGTTTAAGGCTCGTGTTGAGACTTCTC |
| | | GTTTTCCTCGTTCTAAGAAGCAGCGTATTCTTCTTCAGAAGTTTGAT |
| | | GAGGATGGTTTTAAGGAGCGTAATCTTAATGATACTCGTTATGTTA |
| | | ATCGTTTTCTTTGTCAGTTTGTTGCTGATCGTATGCGTCTTACTGGTA |
| | | AGGGTAAGAAGCGTGTTTTTGCTTCTAATGGTCAGATTACTAATCTT |
| | | CTTCGTGGTTTTTGGGGTCTTCGTAAGGTTCGTGCTGAGAATGATCG |
| | | TCATCATGCTCTTGATGCTGTTGTTGTTGCTTGTTCTACTGTTGCTAT |
| | | GCAGCAGAAGATTACTCGTTTTGTTCGTTATAAGGAGATGAATGCT |
| | | TTTGATGGTAAGACTATTGATAAGGAGACTGGTGAGGTTCTTCATC |
| | | AGAAGACTCATTTTCCTCAGCCTTGGGAGTTTTTTGCTCAGGAGGTT |
| | | ATGATTCGTGTTTTTGGTAAGCCTGATGGTAAGCCTGAGTTTGAGGA |
| | | GGCTGATACTCTTGAGAAGCTTCGTACTCTTCTTGCTGAGAAGCTTT |
| | | CTTCTCGTCCTGAGGCTGTTCATGAGTATGTTACTCCTCTTTTTGTTT |
| | | CTCGTGCTCCTAATCGTAAGATGTCTGGTCAGGGTCATATGGAGAC |
| | | TGTTAAGTCTGCTAAGCGTCTTGATGAGGGTGTTTCTGTTCTTCGTG |
| | | TTCCTCTTACTCAGCTTAAGCTTAAGGATCTTGAGAAGATGGTTAAT |
| | | CGTGAGCGTGAGCCTAAGCTTTATGAGGCTCTTAAGGCTCGTCTTG |
| | | AGGCTCATAAGGATGATCCTGCTAAGGCTTTTGCTGAGCCTTTTTAT |
| | | AAGTATGATAAGGCTGGTAATCGTACTCAGCAGGTTAAGGCTGTTC |
| | | GTGTTGAGCAGGTTCAGAAGACTGGTGTTTGGGTTCGTAATCATAA |
| | | TGGTATTGCTGATAATGCTACTATGGTTCGTGTTGATGTTTTTGAGA |
| | | AGGGTGATAAGTATTATCTTGTTCCTATTTATTCTTGGCAGGTTGCT |
| | | AAGGGTATTCTTCCTGATCGTGCTGTTGTTCAGGGTAAGGATGAGG |
| | | AGGATTGGCAGCTTATTGATGATTCTTTTAATTTTAAGTTTTCTCTTC |
| | | ATCCTAATGATCTTGTTGAGGTTATTACTAAGAAGGCTCGTATGTTT |
| | | GGTTATTTTGCTTCTTGTCATCGTGGTACTGGTAATATTAATATTCGT |
| | | ATTCATGATCTTGATCATAAGATTGGTAAGAATGGTATTCTTGAGG |
| | | GTATTGGTGTTAAGACTGCTCTTTCTTTTCAGAAGTATCAGATTGAT |
| | | GAGCTTGGTAAGGAGATTCGTCCTTGTCGTCTTAAGAAGCGTCCTCC |
| | | TGTTCGT |
| 193 | Exemplary | GCCGCCTTCAAGCCCAACTCCATCAACTACATCCTGGGCCTGGACA |
| | coding sequence | TCGGCATCGCCTCCGTGGGCTGGGCCATGGTGGAGATCGACGAGGA |
| | encoding | GGAGAACCCCATCCGGCTGATCGACCTGGGCGTGCGGGTGTTCGAG |
| | Nme1Cas9 | CGGGCCGAGGTGCCCAAGACCGGCGACTCCCTGGCCATGGCCCGGC |
| | cleavase | GGCTGGCCCGGTCCGTGCGGCGGCTGACCCGGCGGCGGGCCCACCG |
| | | GCTGCTGCGGACCCGGCGGCTGCTGAAGCGGGAGGGCGTGCTGCA |
| | | GGCCGCCAACTTCGACGAGAACGGCCTGATCAAGTCCCTGCCCAAC |
| | | ACCCCCTGGCAGCTGCGGGCCGCCGCCCTGGACCGGAAGCTGACCC |
| | | CCCTGGAGTGGTCCGCCGTGCTGCTGCACCTGATCAAGCACCGGGG |
| | | CTACCTGTCCCAGCGGAAGAACGAGGGCGAGACCGCCGACAAGGA |
| | | GCTGGGCGCCCTGCTGAAGGGCGTGGCCGGCAACGCCCACGCCCTG |
| | | CAGACCGGCGACTTCCGGACCCCCGCCGAGCTGGCCCTGAACAAGT |
| | | TCGAGAAGGAGTCCGGCCACATCCGGAACCAGCGGTCCGACTACTC |
| | | CCACACCTTCTCCCGGAAGGACCTGCAGGCCGAGCTGATCCTGCTG |
| | | TTCGAGAAGCAGAAGGAGTTCGGCAACCCCCACGTGTCCGGCGGCC |
| | | TGAAGGAGGGCATCGAGACCCTGCTGATGACCCAGCGGCCCGCCCT |
| | | GTCCGGCGACGCCGTGCAGAAGATGCTGGGCCACTGCACCTTCGAG |
| | | CCCGCCGAGCCCAAGGCCGCCAAGAACACCTACACCGCCGAGCGG |
| | | TTCATCTGGCTGACCAAGCTGAACAACCTGCGGATCCTGGAGCAGG |
| | | GCTCCGAGCGGCCCCTGACCGACACCGAGCGGGCCACCCTGATGGA |
| | | CGAGCCCTACCGGAAGTCCAAGCTGACCTACGCCCAGGCCCGGAAG |
| | | CTGCTGGGCCTGGAGGACACCGCCTTCTTCAAGGGCCTGCGGTACG |
| | | GCAAGGACAACGCCGAGGCCTCCACCCTGATGGAGATGAAGGCCT |
| | | ACCACGCCATCTCCCGGGCCCTGGAGAAGGAGGGCCTGAAGGACA |
| | | AGAAGTCCCCCCTGAACCTGTCCCCCGAGCTGCAGGACGAGATCGG |
| | | CACCGCCTTCTCCCTGTTCAAGACCGACGAGGACATCACCGGCCGG |
| | | CTGAAGGACCGGATCCAGCCCGAGATCCTGGAGGCCCTGCTGAAGC |
| | | ACATCTCCTTCGACAAGTTCGTGCAGATCTCCCTGAAGGCCCTGCG |
| | | GCGGATCGTGCCCCTGATGGAGCAGGGCAAGCGGTACGACGAGGC |
| | | CTGCGCCGAGATCTACGGCGACCACTACGGCAAGAAGAACACCGA |
| | | GGAGAAGATCTACCTGCCCCCCATCCCCGCCGACGAGATCCGGAAC |
| | | CCCGTGGTGCTGCGGGCCCTGTCCCAGGCCCGGAAGGTGATCAACG |
| | | GCGTGGTGCGGCGGTACGGCTCCCCCGCCCGGATCCACATCGAGAC |
| | | CGCCCGGGAGGTGGGCAAGTCCTTCAAGGACCGGAAGGAGATCGA |
| | | GAAGCGGCAGGAGGAGAACCGGAAGGACCGGGAGAAGGCCGCCG |
| | | CCAAGTTCCGGGAGTACTTCCCCAACTTCGTGGGCGAGCCCAAGTC |
| | | CAAGGACATCCTGAAGCTGCGGCTGTACGAGCAGCAGCACGGCAA |
| | | GTGCCTGTACTCCGGCAAGGAGATCAACCTGGGCCGGCTGAACGAG |
| | | AAGGGCTACGTGGAGATCGACCACGCCCTGCCCTTCTCCCGGACCT |
| | | GGGACGACTCCTTCAACAACAAGGTGCTGGTGCTGGGCTCCGAGAA |
| | | CCAGAACAAGGGCAACCAGACCCCCTACGAGTACTTCAACGGCAA |
| | | GGACAACTCCCGGGAGTGGCAGGAGTTCAAGGCCCGGGTGGAGAC |
| | | CTCCCGGTTCCCCCGGTCCAAGAAGCAGCGGATCCTGCTGCAGAAG |
| | | TTCGACGAGGACGGCTTCAAGGAGCGGAACCTGAACGACACCCGG |
| | | TACGTGAACCGGTTCCTGTGCCAGTTCGTGGCCGACCGGATGCGGC |
| | | TGACCGGCAAGGGCAAGAAGCGGGTGTTCGCCTCCAACGGCCAGA |
| | | TCACCAACCTGCTGCGGGGCTTCTGGGGCCTGCGGAAGGTGCGGGC |
| | | CGAGAACGACCGGCACCACGCCCTGGACGCCGTGGTGGTGGCCTGC |
| | | TCCACCGTGGCCATGCAGCAGAAGATCACCCGGTTCGTGCGGTACA |
| | | AGGAGATGAACGCCTTCGACGGCAAGACCATCGACAAGGAGACCG |
| | | GCGAGGTGCTGCACCAGAAGACCCACTTCCCCCAGCCCTGGGAGTT |
| | | CTTCGCCCAGGAGGTGATGATCCGGGTGTTCGGCAAGCCCGACGGC |
| | | AAGCCCGAGTTCGAGGAGGCCGACACCCTGGAGAAGCTGCGGACC |
| | | CTGCTGGCCGAGAAGCTGTCCTCCCGGCCCGAGGCCGTGCACGAGT |
| | | ACGTGACCCCCCTGTTCGTGTCCCGGGCCCCCAACCGGAAGATGTC |
| | | CGGCCAGGGCCACATGGAGACCGTGAAGTCCGCCAAGCGGCTGGA |
| | | CGAGGGCGTGTCCGTGCTGCGGGTGCCCCTGACCCAGCTGAAGCTG |
| | | AAGGACCTGGAGAAGATGGTGAACCGGGAGCGGGAGCCCAAGCTG |
| | | TACGAGGCCCTGAAGGCCCGGCTGGAGGCCCACAAGGACGACCCC |
| | | GCCAAGGCCTTCGCCGAGCCCTTCTACAAGTACGACAAGGCCGGCA |
| | | ACCGGACCCAGCAGGTGAAGGCCGTGCGGGTGGAGCAGGTGCAGA |
| | | AGACCGGCGTGTGGGTGCGGAACCACAACGGCATCGCCGACAACG |
| | | CCACCATGGTGCGGGTGGACGTGTTCGAGAAGGGCGACAAGTACTA |
| | | CCTGGTGCCCATCTACTCCTGGCAGGTGGCCAAGGGCATCCTGCCC |
| | | GACCGGGCCGTGGTGCAGGGCAAGGACGAGGAGGACTGGCAGCTG |
| | | ATCGACGACTCCTTCAACTTCAAGTTCTCCCTGCACCCCAACGACCT |
| | | GGTGGAGGTGATCACCAAGAAGGCCCGGATGTTCGGCTACTTCGCC |
| | | TCCTGCCACCGGGGCACCGGCAACATCAACATCCGGATCCACGACC |
| | | TGGACCACAAGATCGGCAAGAACGGCATCCTGGAGGGCATCGGCG |
| | | TGAAGACCGCCCTGTCCTTCCAGAAGTACCAGATCGACGAGCTGGG |
| | | CAAGGAGATCCGGCCCTGCCGGCTGAAGAAGCGGCCCCCCGTGCG |
| | | G |
| 194 | Exemplary | GCAGCATTCAAACCAAACTCAATCAACTACATCCTAGGACTAGACA |
| | coding sequence | TCGGAATCGCATCAGTAGGATGAGCAATGGTAGAAATCGACGAAG |
| | encoding | AAGAAAACCCAATCCGACTAATCGACCTAGGAGTACGAGTATTCGA |
| | Nme1Cas9 | ACGAGCAGAAGTACCAAAAACAGGAGACTCACTAGCAATGGCACG |
| | cleavase | ACGACTAGCACGATCAGTACGACGACTAACACGACGACGAGCACA |
| | | CCGACTACTACGAACACGACGACTACTAAAACGAGAAGGAGTACT |
| | | ACAAGCAGCAAACTTCGACGAAAACGGACTAATCAAATCACTACC |
| | | AAACACACCATGACAACTACGAGCAGCAGCACTAGACCGAAAACT |
| | | AACACCACTAGAATGATCAGCAGTACTACTACACCTAATCAAACAC |
| | | CGAGGATACCTATCACAACGAAAAAACGAAGGAGAAACAGCAGAC |
| | | AAAGAACTAGGAGCACTACTAAAAGGAGTAGCAGGAAACGCACAC |
| | | GCACTACAAACAGGAGACTTCCGAACACCAGCAGAACTAGCACTA |
| | | AACAAATTCGAAAAAGAATCAGGACACATCCGAAACCAACGATCA |
| | | GACTACTCACACACATTCTCACGAAAAGACCTACAAGCAGAACTAA |
| | | TCCTACTATTCGAAAAACAAAAAGAATTCGGAAACCCACACGTATC |
| | | AGGAGGACTAAAAGAAGGAATCGAAACACTACTAATGACACAACG |
| | | ACCAGCACTATCAGGAGACGCAGTACAAAAAATGCTAGGACACTG |
| | | CACATTCGAACCAGCAGAACCAAAAGCAGCAAAAAACACATACAC |
| | | AGCAGAACGATTCATCTGACTAACAAAACTAAACAACCTACGAATC |
| | | CTAGAACAAGGATCAGAACGACCACTAACAGACACAGAACGAGCA |
| | | ACACTAATGGACGAACCATACCGAAAATCAAAACTAACATACGCA |
| | | CAAGCACGAAAACTACTAGGACTAGAAGACACAGCATTCTTCAAA |
| | | GGACTACGATACGGAAAAGACAACGCAGAAGCATCAACACTAATG |
| | | GAAATGAAAGCATACCACGCAATCTCACGAGCACTAGAAAAAGAA |
| | | GGACTAAAAGACAAAAAATCACCACTAAACCTATCACCAGAACTA |
| | | CAAGACGAAATCGGAACAGCATTCTCACTATTCAAAACAGACGAA |
| | | GACATCACAGGACGACTAAAAGACCGAATCCAACCAGAAATCCTA |
| | | GAAGCACTACTAAAACACATCTCATTCGACAAATTCGTACAAATCT |
| | | CACTAAAAGCACTACGACGAATCGTACCACTAATGGAACAAGGAA |
| | | AACGATACGACGAAGCATGCGCAGAAATCTACGGAGACCACTACG |
| | | GAAAAAAAAACACAGAAGAAAAAATCTACCTACCACCAATCCCAG |
| | | CAGACGAAATCCGAAACCCAGTAGTACTACGAGCACTATCACAAGC |
| | | ACGAAAAGTAATCAACGGAGTAGTACGACGATACGGATCACCAGC |
| | | ACGAATCCACATCGAAACAGCACGAGAAGTAGGAAAATCATTCAA |
| | | AGACCGAAAAGAAATCGAAAAACGACAAGAAGAAAACCGAAAAG |
| | | ACCGAGAAAAAGCAGCAGCAAAATTCCGAGAATACTTCCCAAACTT |
| | | CGTAGGAGAACCAAAATCAAAAGACATCCTAAAACTACGACTATA |
| | | CGAACAACAACACGGAAAATGCCTATACTCAGGAAAAGAAATCAA |
| | | CCTAGGACGACTAAACGAAAAAGGATACGTAGAAATCGACCACGC |
| | | ACTACCATTCTCACGAACATGAGACGACTCATTCAACAACAAAGTA |
| | | CTAGTACTAGGATCAGAAAACCAAAACAAAGGAAACCAAACACCA |
| | | TACGAATACTTCAACGGAAAAGACAACTCACGAGAATGACAAGAA |
| | | TTCAAAGCACGAGTAGAAACATCACGATTCCCACGATCAAAAAAAC |
| | | AACGAATCCTACTACAAAAATTCGACGAAGACGGATTCAAAGAAC |
| | | GAAACCTAAACGACACACGATACGTAAACCGATTCCTATGCCAATT |
| | | CGTAGCAGACCGAATGCGACTAACAGGAAAAGGAAAAAAACGAGT |
| | | ATTCGCATCAAACGGACAAATCACAAACCTACTACGAGGATTCTGA |
| | | GGACTACGAAAAGTACGAGCAGAAAACGACCGACACCACGCACTA |
| | | GACGCAGTAGTAGTAGCATGCTCAACAGTAGCAATGCAACAAAAA |
| | | ATCACACGATTCGTACGATACAAAGAAATGAACGCATTCGACGGAA |
| | | AAACAATCGACAAAGAAACAGGAGAAGTACTACACCAAAAAACAC |
| | | ACTTCCCACAACCATGAGAATTCTTCGCACAAGAAGTAATGATCCG |
| | | AGTATTCGGAAAACCAGACGGAAAACCAGAATTCGAAGAAGCAGA |
| | | CACACTAGAAAAACTACGAACACTACTAGCAGAAAAACTATCATCA |
| | | CGACCAGAAGCAGTACACGAATACGTAACACCACTATTCGTATCAC |
| | | GAGCACCAAACCGAAAAATGTCAGGACAAGGACACATGGAAACAG |
| | | TAAAATCAGCAAAACGACTAGACGAAGGAGTATCAGTACTACGAG |
| | | TACCACTAACACAACTAAAACTAAAAGACCTAGAAAAAATGGTAA |
| | | ACCGAGAACGAGAACCAAAACTATACGAAGCACTAAAAGCACGAC |
| | | TAGAAGCACACAAAGACGACCCAGCAAAAGCATTCGCAGAACCAT |
| | | TCTACAAATACGACAAAGCAGGAAACCGAACACAACAAGTAAAAG |
| | | CAGTACGAGTAGAACAAGTACAAAAAACAGGAGTATGAGTACGAA |
| | | ACCACAACGGAATCGCAGACAACGCAACAATGGTACGAGTAGACG |
| | | TATTCGAAAAAGGAGACAAATACTACCTAGTACCAATCTACTCATG |
| | | ACAAGTAGCAAAAGGAATCCTACCAGACCGAGCAGTAGTACAAGG |
| | | AAAAGACGAAGAAGACTGACAACTAATCGACGACTCATTCAACTTC |
| | | AAATTCTCACTACACCCAAACGACCTAGTAGAAGTAATCACAAAAA |
| | | AAGCACGAATGTTCGGATACTTCGCATCATGCCACCGAGGAACAGG |
| | | AAACATCAACATCCGAATCCACGACCTAGACCACAAAATCGGAAA |
| | | AAACGGAATCCTAGAAGGAATCGGAGTAAAAACAGCACTATCATT |
| | | CCAAAAATACCAAATCGACGAACTAGGAAAAGAAATCCGACCATG |
| | | CCGACTAAAAAAACGACCACCAGTACGA |
| 195 | Exemplary open | ATGGCTGCTTTTAAGCCTAATTCTATTAATTATATTCTTGGTCTTGAT |
| | reading frame for | ATTGGTATTGCTTCTGTTGGTTGGGCTATGGTTGAGATTGATGAGGA |
| | Nme1Cas9 | GGAGAATCCTATTCGTCTTATTGATCTTGGTGTTCGTGTTTTTGAGC |
| | cleavase | GTGCTGAGGTTCCTAAGACTGGTGATTCTCTTGCTATGGCTCGTCGT |
| | | CTTGCTCGTTCTGTTCGTCGTCTTACTCGTCGTCGTGCTCATCGTCTT |
| | | CTTCGTACTCGTCGTCTTCTTAAGCGTGAGGGTGTTCTTCAGGCTGC |
| | | TAATTTTGATGAGAATGGTCTTATTAAGTCTCTTCCTAATACTCCTT |
| | | GGCAGCTTCGTGCTGCTGCTCTTGATCGTAAGCTTACTCCTCTTGAG |
| | | TGGTCTGCTGTTCTTCTTCATCTTATTAAGCATCGTGGTTATCTTTCT |
| | | CAGCGTAAGAATGAGGGTGAGACTGCTGATAAGGAGCTTGGTGCTC |
| | | TTCTTAAGGGTGTTGCTGGTAATGCTCATGCTCTTCAGACTGGTGAT |
| | | TTTCGTACTCCTGCTGAGCTTGCTCTTAATAAGTTTGAGAAGGAGTC |
| | | TGGTCATATTCGTAATCAGCGTTCTGATTATTCTCATACTTTTTCTCG |
| | | TAAGGATCTTCAGGCTGAGCTTATTCTTCTTTTTGAGAAGCAGAAGG |
| | | AGTTTGGTAATCCTCATGTTTCTGGTGGTCTTAAGGAGGGTATTGAG |
| | | ACTCTTCTTATGACTCAGCGTCCTGCTCTTTCTGGTGATGCTGTTCA |
| | | GAAGATGCTTGGTCATTGTACTTTTGAGCCTGCTGAGCCTAAGGCTG |
| | | CTAAGAATACTTATACTGCTGAGCGTTTTATTTGGCTTACTAAGCTT |
| | | AATAATCTTCGTATTCTTGAGCAGGGTTCTGAGCGTCCTCTTACTGA |
| | | TACTGAGCGTGCTACTCTTATGGATGAGCCTTATCGTAAGTCTAAGC |
| | | TTACTTATGCTCAGGCTCGTAAGCTTCTTGGTCTTGAGGATACTGCT |
| | | TTTTTTAAGGGTCTTCGTTATGGTAAGGATAATGCTGAGGCTTCTAC |
| | | TCTTATGGAGATGAAGGCTTATCATGCTATTTCTCGTGCTCTTGAGA |
| | | AGGAGGGTCTTAAGGATAAGAAGTCTCCTCTTAATCTTTCTCCTGAG |
| | | CTTCAGGATGAGATTGGTACTGCTTTTTCTCTTTTTAAGACTGATGA |
| | | GGATATTACTGGTCGTCTTAAGGATCGTATTCAGCCTGAGATTCTTG |
| | | AGGCTCTTCTTAAGCATATTTCTTTTGATAAGTTTGTTCAGATTTCTC |
| | | TTAAGGCTCTTCGTCGTATTGTTCCTCTTATGGAGCAGGGTAAGCGT |
| | | TATGATGAGGCTTGTGCTGAGATTTATGGTGATCATTATGGTAAGA |
| | | AGAATACTGAGGAGAAGATTTATCTTCCTCCTATTCCTGCTGATGAG |
| | | ATTCGTAATCCTGTTGTTCTTCGTGCTCTTTCTCAGGCTCGTAAGGTT |
| | | ATTAATGGTGTTGTTCGTCGTTATGGTTCTCCTGCTCGTATTCATATT |
| | | GAGACTGCTCGTGAGGTTGGTAAGTCTTTTAAGGATCGTAAGGAGA |
| | | TTGAGAAGCGTCAGGAGGAGAATCGTAAGGATCGTGAGAAGGCTG |
| | | CTGCTAAGTTTCGTGAGTATTTTCCTAATTTTGTTGGTGAGCCTAAG |
| | | TCTAAGGATATTCTTAAGCTTCGTCTTTATGAGCAGCAGCATGGTAA |
| | | GTGTCTTTATTCTGGTAAGGAGATTAATCTTGGTCGTCTTAATGAGA |
| | | AGGGTTATGTTGAGATTGATCATGCTCTTCCTTTTTCTCGTACTTGG |
| | | GATGATTCTTTTAATAATAAGGTTCTTGTTCTTGGTTCTGAGAATCA |
| | | GAATAAGGGTAATCAGACTCCTTATGAGTATTTTAATGGTAAGGAT |
| | | AATTCTCGTGAGTGGCAGGAGTTTAAGGCTCGTGTTGAGACTTCTC |
| | | GTTTTCCTCGTTCTAAGAAGCAGCGTATTCTTCTTCAGAAGTTTGAT |
| | | GAGGATGGTTTTAAGGAGCGTAATCTTAATGATACTCGTTATGTTA |
| | | ATCGTTTTCTTTGTCAGTTTGTTGCTGATCGTATGCGTCTTACTGGTA |
| | | AGGGTAAGAAGCGTGTTTTTGCTTCTAATGGTCAGATTACTAATCTT |
| | | CTTCGTGGTTTTTGGGGTCTTCGTAAGGTTCGTGCTGAGAATGATCG |
| | | TCATCATGCTCTTGATGCTGTTGTTGTTGCTTGTTCTACTGTTGCTAT |
| | | GCAGCAGAAGATTACTCGTTTTGTTCGTTATAAGGAGATGAATGCT |
| | | TTTGATGGTAAGACTATTGATAAGGAGACTGGTGAGGTTCTTCATC |
| | | AGAAGACTCATTTTCCTCAGCCTTGGGAGTTTTTTGCTCAGGAGGTT |
| | | ATGATTCGTGTTTTTGGTAAGCCTGATGGTAAGCCTGAGTTTGAGGA |
| | | GGCTGATACTCTTGAGAAGCTTCGTACTCTTCTTGCTGAGAAGCTTT |
| | | CTTCTCGTCCTGAGGCTGTTCATGAGTATGTTACTCCTCTTTTTGTTT |
| | | CTCGTGCTCCTAATCGTAAGATGTCTGGTCAGGGTCATATGGAGAC |
| | | TGTTAAGTCTGCTAAGCGTCTTGATGAGGGTGTTTCTGTTCTTCGTG |
| | | TTCCTCTTACTCAGCTTAAGCTTAAGGATCTTGAGAAGATGGTTAAT |
| | | CGTGAGCGTGAGCCTAAGCTTTATGAGGCTCTTAAGGCTCGTCTTG |
| | | AGGCTCATAAGGATGATCCTGCTAAGGCTTTTGCTGAGCCTTTTTAT |
| | | AAGTATGATAAGGCTGGTAATCGTACTCAGCAGGTTAAGGCTGTTC |
| | | GTGTTGAGCAGGTTCAGAAGACTGGTGTTTGGGTTCGTAATCATAA |
| | | TGGTATTGCTGATAATGCTACTATGGTTCGTGTTGATGTTTTTGAGA |
| | | AGGGTGATAAGTATTATCTTGTTCCTATTTATTCTTGGCAGGTTGCT |
| | | AAGGGTATTCTTCCTGATCGTGCTGTTGTTCAGGGTAAGGATGAGG |
| | | AGGATTGGCAGCTTATTGATGATTCTTTTAATTTTAAGTTTTCTCTTC |
| | | ATCCTAATGATCTTGTTGAGGTTATTACTAAGAAGGCTCGTATGTTT |
| | | GGTTATTTTGCTTCTTGTCATCGTGGTACTGGTAATATTAATATTCGT |
| | | ATTCATGATCTTGATCATAAGATTGGTAAGAATGGTATTCTTGAGG |
| | | GTATTGGTGTTAAGACTGCTCTTTCTTTTCAGAAGTATCAGATTGAT |
| | | GAGCTTGGTAAGGAGATTCGTCCTTGTCGTCTTAAGAAGCGTCCTCC |
| | | TGTTCGTUGA |
| 196 | Exemplary open | ATGGCCGCCTTCAAGCCCAACTCCATCAACTACATCCTGGGCCTGG |
| | reading frame for | ACATCGGCATCGCCTCCGTGGGCTGGGCCATGGTGGAGATCGACGA |
| | Nme1Cas9 | GGAGGAGAACCCCATCCGGCTGATCGACCTGGGCGTGCGGGTGTTC |
| | cleavase | GAGCGGGCCGAGGTGCCCAAGACCGGCGACTCCCTGGCCATGGCCC |
| | | GGCGGCTGGCCCGGTCCGTGCGGCGGCTGACCCGGCGGCGGGCCCA |
| | | CCGGCTGCTGCGGACCCGGCGGCTGCTGAAGCGGGAGGGCGTGCTG |
| | | CAGGCCGCCAACTTCGACGAGAACGGCCTGATCAAGTCCCTGCCCA |
| | | ACACCCCCTGGCAGCTGCGGGCCGCCGCCCTGGACCGGAAGCTGAC |
| | | CCCCCTGGAGTGGTCCGCCGTGCTGCTGCACCTGATCAAGCACCGG |
| | | GGCTACCTGTCCCAGCGGAAGAACGAGGGCGAGACCGCCGACAAG |
| | | GAGCTGGGCGCCCTGCTGAAGGGCGTGGCCGGCAACGCCCACGCCC |
| | | TGCAGACCGGCGACTTCCGGACCCCCGCCGAGCTGGCCCTGAACAA |
| | | GTTCGAGAAGGAGTCCGGCCACATCCGGAACCAGCGGTCCGACTAC |
| | | TCCCACACCTTCTCCCGGAAGGACCTGCAGGCCGAGCTGATCCTGC |
| | | TGTTCGAGAAGCAGAAGGAGTTCGGCAACCCCCACGTGTCCGGCGG |
| | | CCTGAAGGAGGGCATCGAGACCCTGCTGATGACCCAGCGGCCCGCC |
| | | CTGTCCGGCGACGCCGTGCAGAAGATGCTGGGCCACTGCACCTTCG |
| | | AGCCCGCCGAGCCCAAGGCCGCCAAGAACACCTACACCGCCGAGC |
| | | GGTTCATCTGGCTGACCAAGCTGAACAACCTGCGGATCCTGGAGCA |
| | | GGGCTCCGAGCGGCCCCTGACCGACACCGAGCGGGCCACCCTGATG |
| | | GACGAGCCCTACCGGAAGTCCAAGCTGACCTACGCCCAGGCCCGGA |
| | | AGCTGCTGGGCCTGGAGGACACCGCCTTCTTCAAGGGCCTGCGGTA |
| | | CGGCAAGGACAACGCCGAGGCCTCCACCCTGATGGAGATGAAGGC |
| | | CTACCACGCCATCTCCCGGGCCCTGGAGAAGGAGGGCCTGAAGGAC |
| | | AAGAAGTCCCCCCTGAACCTGTCCCCCGAGCTGCAGGACGAGATCG |
| | | GCACCGCCTTCTCCCTGTTCAAGACCGACGAGGACATCACCGGCCG |
| | | GCTGAAGGACCGGATCCAGCCCGAGATCCTGGAGGCCCTGCTGAAG |
| | | CACATCTCCTTCGACAAGTTCGTGCAGATCTCCCTGAAGGCCCTGCG |
| | | GCGGATCGTGCCCCTGATGGAGCAGGGCAAGCGGTACGACGAGGC |
| | | CTGCGCCGAGATCTACGGCGACCACTACGGCAAGAAGAACACCGA |
| | | GGAGAAGATCTACCTGCCCCCCATCCCCGCCGACGAGATCCGGAAC |
| | | CCCGTGGTGCTGCGGGCCCTGTCCCAGGCCCGGAAGGTGATCAACG |
| | | GCGTGGTGCGGCGGTACGGCTCCCCCGCCCGGATCCACATCGAGAC |
| | | CGCCCGGGAGGTGGGCAAGTCCTTCAAGGACCGGAAGGAGATCGA |
| | | GAAGCGGCAGGAGGAGAACCGGAAGGACCGGGAGAAGGCCGCCG |
| | | CCAAGTTCCGGGAGTACTTCCCCAACTTCGTGGGCGAGCCCAAGTC |
| | | CAAGGACATCCTGAAGCTGCGGCTGTACGAGCAGCAGCACGGCAA |
| | | GTGCCTGTACTCCGGCAAGGAGATCAACCTGGGCCGGCTGAACGAG |
| | | AAGGGCTACGTGGAGATCGACCACGCCCTGCCCTTCTCCCGGACCT |
| | | GGGACGACTCCTTCAACAACAAGGTGCTGGTGCTGGGCTCCGAGAA |
| | | CCAGAACAAGGGCAACCAGACCCCCTACGAGTACTTCAACGGCAA |
| | | GGACAACTCCCGGGAGTGGCAGGAGTTCAAGGCCCGGGTGGAGAC |
| | | CTCCCGGTTCCCCCGGTCCAAGAAGCAGCGGATCCTGCTGCAGAAG |
| | | TTCGACGAGGACGGCTTCAAGGAGCGGAACCTGAACGACACCCGG |
| | | TACGTGAACCGGTTCCTGTGCCAGTTCGTGGCCGACCGGATGCGGC |
| | | TGACCGGCAAGGGCAAGAAGCGGGTGTTCGCCTCCAACGGCCAGA |
| | | TCACCAACCTGCTGCGGGGCTTCTGGGGCCTGCGGAAGGTGCGGGC |
| | | CGAGAACGACCGGCACCACGCCCTGGACGCCGTGGTGGTGGCCTGC |
| | | TCCACCGTGGCCATGCAGCAGAAGATCACCCGGTTCGTGCGGTACA |
| | | AGGAGATGAACGCCTTCGACGGCAAGACCATCGACAAGGAGACCG |
| | | GCGAGGTGCTGCACCAGAAGACCCACTTCCCCCAGCCCTGGGAGTT |
| | | CTTCGCCCAGGAGGTGATGATCCGGGTGTTCGGCAAGCCCGACGGC |
| | | AAGCCCGAGTTCGAGGAGGCCGACACCCTGGAGAAGCTGCGGACC |
| | | CTGCTGGCCGAGAAGCTGTCCTCCCGGCCCGAGGCCGTGCACGAGT |
| | | ACGTGACCCCCCTGTTCGTGTCCCGGGCCCCCAACCGGAAGATGTC |
| | | CGGCCAGGGCCACATGGAGACCGTGAAGTCCGCCAAGCGGCTGGA |
| | | CGAGGGCGTGTCCGTGCTGCGGGTGCCCCTGACCCAGCTGAAGCTG |
| | | AAGGACCTGGAGAAGATGGTGAACCGGGAGCGGGAGCCCAAGCTG |
| | | TACGAGGCCCTGAAGGCCCGGCTGGAGGCCCACAAGGACGACCCC |
| | | GCCAAGGCCTTCGCCGAGCCCTTCTACAAGTACGACAAGGCCGGCA |
| | | ACCGGACCCAGCAGGTGAAGGCCGTGCGGGTGGAGCAGGTGCAGA |
| | | AGACCGGCGTGTGGGTGCGGAACCACAACGGCATCGCCGACAACG |
| | | CCACCATGGTGCGGGTGGACGTGTTCGAGAAGGGCGACAAGTACTA |
| | | CCTGGTGCCCATCTACTCCTGGCAGGTGGCCAAGGGCATCCTGCCC |
| | | GACCGGGCCGTGGTGCAGGGCAAGGACGAGGAGGACTGGCAGCTG |
| | | ATCGACGACTCCTTCAACTTCAAGTTCTCCCTGCACCCCAACGACCT |
| | | GGTGGAGGTGATCACCAAGAAGGCCCGGATGTTCGGCTACTTCGCC |
| | | TCCTGCCACCGGGGCACCGGCAACATCAACATCCGGATCCACGACC |
| | | TGGACCACAAGATCGGCAAGAACGGCATCCTGGAGGGCATCGGCG |
| | | TGAAGACCGCCCTGTCCTTCCAGAAGTACCAGATCGACGAGCTGGG |
| | | CAAGGAGATCCGGCCCTGCCGGCTGAAGAAGCGGCCCCCCGTGCG |
| | | GUGA |
| 197 | Exemplary open | ATGGCAGCATTCAAACCAAACTCAATCAACTACATCCTAGGACTAG |
| | reading frame for | ACATCGGAATCGCATCAGTAGGATGAGCAATGGTAGAAATCGACG |
| | Nme1Cas9 | AAGAAGAAAACCCAATCCGACTAATCGACCTAGGAGTACGAGTATT |
| | cleavase | CGAACGAGCAGAAGTACCAAAAACAGGAGACTCACTAGCAATGGC |
| | | ACGACGACTAGCACGATCAGTACGACGACTAACACGACGACGAGC |
| | | ACACCGACTACTACGAACACGACGACTACTAAAACGAGAAGGAGT |
| | | ACTACAAGCAGCAAACTTCGACGAAAACGGACTAATCAAATCACTA |
| | | CCAAACACACCATGACAACTACGAGCAGCAGCACTAGACCGAAAA |
| | | CTAACACCACTAGAATGATCAGCAGTACTACTACACCTAATCAAAC |
| | | ACCGAGGATACCTATCACAACGAAAAAACGAAGGAGAAACAGCAG |
| | | ACAAAGAACTAGGAGCACTACTAAAAGGAGTAGCAGGAAACGCAC |
| | | ACGCACTACAAACAGGAGACTTCCGAACACCAGCAGAACTAGCAC |
| | | TAAACAAATTCGAAAAAGAATCAGGACACATCCGAAACCAACGAT |
| | | CAGACTACTCACACACATTCTCACGAAAAGACCTACAAGCAGAACT |
| | | AATCCTACTATTCGAAAAACAAAAAGAATTCGGAAACCCACACGTA |
| | | TCAGGAGGACTAAAAGAAGGAATCGAAACACTACTAATGACACAA |
| | | CGACCAGCACTATCAGGAGACGCAGTACAAAAAATGCTAGGACAC |
| | | TGCACATTCGAACCAGCAGAACCAAAAGCAGCAAAAAACACATAC |
| | | ACAGCAGAACGATTCATCTGACTAACAAAACTAAACAACCTACGAA |
| | | TCCTAGAACAAGGATCAGAACGACCACTAACAGACACAGAACGAG |
| | | CAACACTAATGGACGAACCATACCGAAAATCAAAACTAACATACG |
| | | CACAAGCACGAAAACTACTAGGACTAGAAGACACAGCATTCTTCAA |
| | | AGGACTACGATACGGAAAAGACAACGCAGAAGCATCAACACTAAT |
| | | GGAAATGAAAGCATACCACGCAATCTCACGAGCACTAGAAAAAGA |
| | | AGGACTAAAAGACAAAAAATCACCACTAAACCTATCACCAGAACT |
| | | ACAAGACGAAATCGGAACAGCATTCTCACTATTCAAAACAGACGA |
| | | AGACATCACAGGACGACTAAAAGACCGAATCCAACCAGAAATCCT |
| | | AGAAGCACTACTAAAACACATCTCATTCGACAAATTCGTACAAATC |
| | | TCACTAAAAGCACTACGACGAATCGTACCACTAATGGAACAAGGA |
| | | AAACGATACGACGAAGCATGCGCAGAAATCTACGGAGACCACTAC |
| | | GGAAAAAAAAACACAGAAGAAAAAATCTACCTACCACCAATCCCA |
| | | GCAGACGAAATCCGAAACCCAGTAGTACTACGAGCACTATCACAA |
| | | GCACGAAAAGTAATCAACGGAGTAGTACGACGATACGGATCACCA |
| | | GCACGAATCCACATCGAAACAGCACGAGAAGTAGGAAAATCATTC |
| | | AAAGACCGAAAAGAAATCGAAAAACGACAAGAAGAAAACCGAAA |
| | | AGACCGAGAAAAAGCAGCAGCAAAATTCCGAGAATACTTCCCAAA |
| | | CTTCGTAGGAGAACCAAAATCAAAAGACATCCTAAAACTACGACTA |
| | | TACGAACAACAACACGGAAAATGCCTATACTCAGGAAAAGAAATC |
| | | AACCTAGGACGACTAAACGAAAAAGGATACGTAGAAATCGACCAC |
| | | GCACTACCATTCTCACGAACATGAGACGACTCATTCAACAACAAAG |
| | | TACTAGTACTAGGATCAGAAAACCAAAACAAAGGAAACCAAACAC |
| | | CATACGAATACTTCAACGGAAAAGACAACTCACGAGAATGACAAG |
| | | AATTCAAAGCACGAGTAGAAACATCACGATTCCCACGATCAAAAA |
| | | AACAACGAATCCTACTACAAAAATTCGACGAAGACGGATTCAAAG |
| | | AACGAAACCTAAACGACACACGATACGTAAACCGATTCCTATGCCA |
| | | ATTCGTAGCAGACCGAATGCGACTAACAGGAAAAGGAAAAAAACG |
| | | AGTATTCGCATCAAACGGACAAATCACAAACCTACTACGAGGATTC |
| | | TGAGGACTACGAAAAGTACGAGCAGAAAACGACCGACACCACGCA |
| | | CTAGACGCAGTAGTAGTAGCATGCTCAACAGTAGCAATGCAACAAA |
| | | AAATCACACGATTCGTACGATACAAAGAAATGAACGCATTCGACGG |
| | | AAAAACAATCGACAAAGAAACAGGAGAAGTACTACACCAAAAAAC |
| | | ACACTTCCCACAACCATGAGAATTCTTCGCACAAGAAGTAATGATC |
| | | CGAGTATTCGGAAAACCAGACGGAAAACCAGAATTCGAAGAAGCA |
| | | GACACACTAGAAAAACTACGAACACTACTAGCAGAAAAACTATCA |
| | | TCACGACCAGAAGCAGTACACGAATACGTAACACCACTATTCGTAT |
| | | CACGAGCACCAAACCGAAAAATGTCAGGACAAGGACACATGGAAA |
| | | CAGTAAAATCAGCAAAACGACTAGACGAAGGAGTATCAGTACTAC |
| | | GAGTACCACTAACACAACTAAAACTAAAAGACCTAGAAAAAATGG |
| | | TAAACCGAGAACGAGAACCAAAACTATACGAAGCACTAAAAGCAC |
| | | GACTAGAAGCACACAAAGACGACCCAGCAAAAGCATTCGCAGAAC |
| | | CATTCTACAAATACGACAAAGCAGGAAACCGAACACAACAAGTAA |
| | | AAGCAGTACGAGTAGAACAAGTACAAAAAACAGGAGTATGAGTAC |
| | | GAAACCACAACGGAATCGCAGACAACGCAACAATGGTACGAGTAG |
| | | ACGTATTCGAAAAAGGAGACAAATACTACCTAGTACCAATCTACTC |
| | | ATGACAAGTAGCAAAAGGAATCCTACCAGACCGAGCAGTAGTACA |
| | | AGGAAAAGACGAAGAAGACTGACAACTAATCGACGACTCATTCAA |
| | | CTTCAAATTCTCACTACACCCAAACGACCTAGTAGAAGTAATCACA |
| | | AAAAAAGCACGAATGTTCGGATACTTCGCATCATGCCACCGAGGAA |
| | | CAGGAAACATCAACATCCGAATCCACGACCTAGACCACAAAATCG |
| | | GAAAAAACGGAATCCTAGAAGGAATCGGAGTAAAAACAGCACTAT |
| | | CATTCCAAAAATACCAAATCGACGAACTAGGAAAAGAAATCCGAC |
| | | CATGCCGACTAAAAAAACGACCACCAGTACGAUAA |
| 198 | Exemplary amino | MAAFKPNSINYILGLDIGIASVGWAMVEIDEEENPIRLIDLGVRVFERAE |
| | acid sequence of | VPKTGDSLAMARRLARSVRRLTRRRAHRLLRTRRLLKREGVLQAANF |
| | Nme1Cas9 HNH | DENGLIKSLPNTPWQLRAAALDRKLTPLEWSAVLLHLIKHRGYLSQRK |
| | nickase | NEGETADKELGALLKGVAGNAHALQTGDFRTPAELALNKFEKESGHI |
| | | RNQRSDYSHTFSRKDLQAELILLFEKQKEFGNPHVSGGLKEGIETLLMT |
| | | QRPALSGDAVQKMLGHCTFEPAEPKAAKNTYTAERFIWLTKLNNLRIL |
| | | EQGSERPLTDTERATLMDEPYRKSKLTYAQARKLLGLEDTAFFKGLRY |
| | | GKDNAEASTLMEMKAYHAISRALEKEGLKDKKSPLNLSPELQDEIGTA |
| | | FSLFKTDEDITGRLKDRIQPEILEALLKHISFDKFVQISLKALRRIVPLME |
| | | QGKRYDEACAEIYGDHYGKKNTEEKIYLPPIPADEIRNPVVLRALSQAR |
| | | KVINGVVRRYGSPARIHIETAREVGKSFKDRKEIEKRQEENRKDREKA |
| | | AAKFREYFPNFVGEPKSKDILKLRLYEQQHGKCLYSGKEINLGRLNEK |
| | | GYVEIDAALPFSRTWDDSFNNKVLVLGSENQNKGNQTPYEYFNGKDN |
| | | SREWQEFKARVETSRFPRSKKQRILLQKFDEDGFKERNLNDTRYVNRF |
| | | LCQFVADRMRLTGKGKKRVFASNGQITNLLRGFWGLRKVRAENDRH |
| | | HALDAVVVACSTVAMQQKITRFVRYKEMNAFDGKTIDKETGEVLHQ |
| | | KTHFPQPWEFFAQEVMIRVFGKPDGKPEFEEADTLEKLRTLLAEKLSSR |
| | | PEAVHEYVTPLFVSRAPNRKMSGQGHMETVKSAKRLDEGVSVLRVPL |
| | | TQLKLKDLEKMVNREREPKLYEALKARLEAHKDDPAKAFAEPFYKYD |
| | | KAGNRTQQVKAVRVEQVQKTGVWVRNHNGIADNATMVRVDVFEKG |
| | | DKYYLVPIYSWQVAKGILPDRAVVQGKDEEDWQLIDDSFNFKFSLHPN |
| | | DLVEVITKKARMFGYFASCHRGTGNINIRIHDLDHKIGKNGILEGIGVK |
| | | TALSFQKYQIDELGKEIRPCRLKKRPPVR |
| 199 | Exemplary | GCTGCTTTTAAGCCTAATTCTATTAATTATATTCTTGGTCTTGATATT |
| | coding sequence | GGTATTGCTTCTGTTGGTTGGGCTATGGTTGAGATTGATGAGGAGG |
| | encoding | AGAATCCTATTCGTCTTATTGATCTTGGTGTTCGTGTTTTTGAGCGT |
| | Nme1Cas9 HNH | GCTGAGGTTCCTAAGACTGGTGATTCTCTTGCTATGGCTCGTCGTCT |
| | nickase | TGCTCGTTCTGTTCGTCGTCTTACTCGTCGTCGTGCTCATCGTCTTCT |
| | | TCGTACTCGTCGTCTTCTTAAGCGTGAGGGTGTTCTTCAGGCTGCTA |
| | | ATTTTGATGAGAATGGTCTTATTAAGTCTCTTCCTAATACTCCTTGG |
| | | CAGCTTCGTGCTGCTGCTCTTGATCGTAAGCTTACTCCTCTTGAGTG |
| | | GTCTGCTGTTCTTCTTCATCTTATTAAGCATCGTGGTTATCTTTCTCA |
| | | GCGTAAGAATGAGGGTGAGACTGCTGATAAGGAGCTTGGTGCTCTT |
| | | CTTAAGGGTGTTGCTGGTAATGCTCATGCTCTTCAGACTGGTGATTT |
| | | TCGTACTCCTGCTGAGCTTGCTCTTAATAAGTTTGAGAAGGAGTCTG |
| | | GTCATATTCGTAATCAGCGTTCTGATTATTCTCATACTTTTTCTCGTA |
| | | AGGATCTTCAGGCTGAGCTTATTCTTCTTTTTGAGAAGCAGAAGGA |
| | | GTTTGGTAATCCTCATGTTTCTGGTGGTCTTAAGGAGGGTATTGAGA |
| | | CTCTTCTTATGACTCAGCGTCCTGCTCTTTCTGGTGATGCTGTTCAG |
| | | AAGATGCTTGGTCATTGTACTTTTGAGCCTGCTGAGCCTAAGGCTGC |
| | | TAAGAATACTTATACTGCTGAGCGTTTTATTTGGCTTACTAAGCTTA |
| | | ATAATCTTCGTATTCTTGAGCAGGGTTCTGAGCGTCCTCTTACTGAT |
| | | ACTGAGCGTGCTACTCTTATGGATGAGCCTTATCGTAAGTCTAAGCT |
| | | TACTTATGCTCAGGCTCGTAAGCTTCTTGGTCTTGAGGATACTGCTT |
| | | TTTTTAAGGGTCTTCGTTATGGTAAGGATAATGCTGAGGCTTCTACT |
| | | CTTATGGAGATGAAGGCTTATCATGCTATTTCTCGTGCTCTTGAGAA |
| | | GGAGGGTCTTAAGGATAAGAAGTCTCCTCTTAATCTTTCTCCTGAGC |
| | | TTCAGGATGAGATTGGTACTGCTTTTTCTCTTTTTAAGACTGATGAG |
| | | GATATTACTGGTCGTCTTAAGGATCGTATTCAGCCTGAGATTCTTGA |
| | | GGCTCTTCTTAAGCATATTTCTTTTGATAAGTTTGTTCAGATTTCTCT |
| | | TAAGGCTCTTCGTCGTATTGTTCCTCTTATGGAGCAGGGTAAGCGTT |
| | | ATGATGAGGCTTGTGCTGAGATTTATGGTGATCATTATGGTAAGAA |
| | | GAATACTGAGGAGAAGATTTATCTTCCTCCTATTCCTGCTGATGAGA |
| | | TTCGTAATCCTGTTGTTCTTCGTGCTCTTTCTCAGGCTCGTAAGGTTA |
| | | TTAATGGTGTTGTTCGTCGTTATGGTTCTCCTGCTCGTATTCATATTG |
| | | AGACTGCTCGTGAGGTTGGTAAGTCTTTTAAGGATCGTAAGGAGAT |
| | | TGAGAAGCGTCAGGAGGAGAATCGTAAGGATCGTGAGAAGGCTGC |
| | | TGCTAAGTTTCGTGAGTATTTTCCTAATTTTGTTGGTGAGCCTAAGT |
| | | CTAAGGATATTCTTAAGCTTCGTCTTTATGAGCAGCAGCATGGTAA |
| | | GTGTCTTTATTCTGGTAAGGAGATTAATCTTGGTCGTCTTAATGAGA |
| | | AGGGTTATGTTGAGATTGATGCTGCTCTTCCTTTTTCTCGTACTTGG |
| | | GATGATTCTTTTAATAATAAGGTTCTTGTTCTTGGTTCTGAGAATCA |
| | | GAATAAGGGTAATCAGACTCCTTATGAGTATTTTAATGGTAAGGAT |
| | | AATTCTCGTGAGTGGCAGGAGTTTAAGGCTCGTGTTGAGACTTCTC |
| | | GTTTTCCTCGTTCTAAGAAGCAGCGTATTCTTCTTCAGAAGTTTGAT |
| | | GAGGATGGTTTTAAGGAGCGTAATCTTAATGATACTCGTTATGTTA |
| | | ATCGTTTTCTTTGTCAGTTTGTTGCTGATCGTATGCGTCTTACTGGTA |
| | | AGGGTAAGAAGCGTGTTTTTGCTTCTAATGGTCAGATTACTAATCTT |
| | | CTTCGTGGTTTTTGGGGTCTTCGTAAGGTTCGTGCTGAGAATGATCG |
| | | TCATCATGCTCTTGATGCTGTTGTTGTTGCTTGTTCTACTGTTGCTAT |
| | | GCAGCAGAAGATTACTCGTTTTGTTCGTTATAAGGAGATGAATGCT |
| | | TTTGATGGTAAGACTATTGATAAGGAGACTGGTGAGGTTCTTCATC |
| | | AGAAGACTCATTTTCCTCAGCCTTGGGAGTTTTTTGCTCAGGAGGTT |
| | | ATGATTCGTGTTTTTGGTAAGCCTGATGGTAAGCCTGAGTTTGAGGA |
| | | GGCTGATACTCTTGAGAAGCTTCGTACTCTTCTTGCTGAGAAGCTTT |
| | | CTTCTCGTCCTGAGGCTGTTCATGAGTATGTTACTCCTCTTTTTGTTT |
| | | CTCGTGCTCCTAATCGTAAGATGTCTGGTCAGGGTCATATGGAGAC |
| | | TGTTAAGTCTGCTAAGCGTCTTGATGAGGGTGTTTCTGTTCTTCGTG |
| | | TTCCTCTTACTCAGCTTAAGCTTAAGGATCTTGAGAAGATGGTTAAT |
| | | CGTGAGCGTGAGCCTAAGCTTTATGAGGCTCTTAAGGCTCGTCTTG |
| | | AGGCTCATAAGGATGATCCTGCTAAGGCTTTTGCTGAGCCTTTTTAT |
| | | AAGTATGATAAGGCTGGTAATCGTACTCAGCAGGTTAAGGCTGTTC |
| | | GTGTTGAGCAGGTTCAGAAGACTGGTGTTTGGGTTCGTAATCATAA |
| | | TGGTATTGCTGATAATGCTACTATGGTTCGTGTTGATGTTTTTGAGA |
| | | AGGGTGATAAGTATTATCTTGTTCCTATTTATTCTTGGCAGGTTGCT |
| | | AAGGGTATTCTTCCTGATCGTGCTGTTGTTCAGGGTAAGGATGAGG |
| | | AGGATTGGCAGCTTATTGATGATTCTTTTAATTTTAAGTTTTCTCTTC |
| | | ATCCTAATGATCTTGTTGAGGTTATTACTAAGAAGGCTCGTATGTTT |
| | | GGTTATTTTGCTTCTTGTCATCGTGGTACTGGTAATATTAATATTCGT |
| | | ATTCATGATCTTGATCATAAGATTGGTAAGAATGGTATTCTTGAGG |
| | | GTATTGGTGTTAAGACTGCTCTTTCTTTTCAGAAGTATCAGATTGAT |
| | | GAGCTTGGTAAGGAGATTCGTCCTTGTCGTCTTAAGAAGCGTCCTCC |
| | | TGTTCGT |
| 200 | Exemplary | GCCGCCTTCAAGCCCAACTCCATCAACTACATCCTGGGCCTGGACA |
| | coding sequence | TCGGCATCGCCTCCGTGGGCTGGGCCATGGTGGAGATCGACGAGGA |
| | encoding | GGAGAACCCCATCCGGCTGATCGACCTGGGCGTGCGGGTGTTCGAG |
| | Nme1Cas9 HNH | CGGGCCGAGGTGCCCAAGACCGGCGACTCCCTGGCCATGGCCCGGC |
| | nickase | GGCTGGCCCGGTCCGTGCGGCGGCTGACCCGGCGGCGGGCCCACCG |
| | | GCTGCTGCGGACCCGGCGGCTGCTGAAGCGGGAGGGCGTGCTGCA |
| | | GGCCGCCAACTTCGACGAGAACGGCCTGATCAAGTCCCTGCCCAAC |
| | | ACCCCCTGGCAGCTGCGGGCCGCCGCCCTGGACCGGAAGCTGACCC |
| | | CCCTGGAGTGGTCCGCCGTGCTGCTGCACCTGATCAAGCACCGGGG |
| | | CTACCTGTCCCAGCGGAAGAACGAGGGCGAGACCGCCGACAAGGA |
| | | GCTGGGCGCCCTGCTGAAGGGCGTGGCCGGCAACGCCCACGCCCTG |
| | | CAGACCGGCGACTTCCGGACCCCCGCCGAGCTGGCCCTGAACAAGT |
| | | TCGAGAAGGAGTCCGGCCACATCCGGAACCAGCGGTCCGACTACTC |
| | | CCACACCTTCTCCCGGAAGGACCTGCAGGCCGAGCTGATCCTGCTG |
| | | TTCGAGAAGCAGAAGGAGTTCGGCAACCCCCACGTGTCCGGCGGCC |
| | | TGAAGGAGGGCATCGAGACCCTGCTGATGACCCAGCGGCCCGCCCT |
| | | GTCCGGCGACGCCGTGCAGAAGATGCTGGGCCACTGCACCTTCGAG |
| | | CCCGCCGAGCCCAAGGCCGCCAAGAACACCTACACCGCCGAGCGG |
| | | TTCATCTGGCTGACCAAGCTGAACAACCTGCGGATCCTGGAGCAGG |
| | | GCTCCGAGCGGCCCCTGACCGACACCGAGCGGGCCACCCTGATGGA |
| | | CGAGCCCTACCGGAAGTCCAAGCTGACCTACGCCCAGGCCCGGAAG |
| | | CTGCTGGGCCTGGAGGACACCGCCTTCTTCAAGGGCCTGCGGTACG |
| | | GCAAGGACAACGCCGAGGCCTCCACCCTGATGGAGATGAAGGCCT |
| | | ACCACGCCATCTCCCGGGCCCTGGAGAAGGAGGGCCTGAAGGACA |
| | | AGAAGTCCCCCCTGAACCTGTCCCCCGAGCTGCAGGACGAGATCGG |
| | | CACCGCCTTCTCCCTGTTCAAGACCGACGAGGACATCACCGGCCGG |
| | | CTGAAGGACCGGATCCAGCCCGAGATCCTGGAGGCCCTGCTGAAGC |
| | | ACATCTCCTTCGACAAGTTCGTGCAGATCTCCCTGAAGGCCCTGCG |
| | | GCGGATCGTGCCCCTGATGGAGCAGGGCAAGCGGTACGACGAGGC |
| | | CTGCGCCGAGATCTACGGCGACCACTACGGCAAGAAGAACACCGA |
| | | GGAGAAGATCTACCTGCCCCCCATCCCCGCCGACGAGATCCGGAAC |
| | | CCCGTGGTGCTGCGGGCCCTGTCCCAGGCCCGGAAGGTGATCAACG |
| | | GCGTGGTGCGGCGGTACGGCTCCCCCGCCCGGATCCACATCGAGAC |
| | | CGCCCGGGAGGTGGGCAAGTCCTTCAAGGACCGGAAGGAGATCGA |
| | | GAAGCGGCAGGAGGAGAACCGGAAGGACCGGGAGAAGGCCGCCG |
| | | CCAAGTTCCGGGAGTACTTCCCCAACTTCGTGGGCGAGCCCAAGTC |
| | | CAAGGACATCCTGAAGCTGCGGCTGTACGAGCAGCAGCACGGCAA |
| | | GTGCCTGTACTCCGGCAAGGAGATCAACCTGGGCCGGCTGAACGAG |
| | | AAGGGCTACGTGGAGATCGACGCCGCCCTGCCCTTCTCCCGGACCT |
| | | GGGACGACTCCTTCAACAACAAGGTGCTGGTGCTGGGCTCCGAGAA |
| | | CCAGAACAAGGGCAACCAGACCCCCTACGAGTACTTCAACGGCAA |
| | | GGACAACTCCCGGGAGTGGCAGGAGTTCAAGGCCCGGGTGGAGAC |
| | | CTCCCGGTTCCCCCGGTCCAAGAAGCAGCGGATCCTGCTGCAGAAG |
| | | TTCGACGAGGACGGCTTCAAGGAGCGGAACCTGAACGACACCCGG |
| | | TACGTGAACCGGTTCCTGTGCCAGTTCGTGGCCGACCGGATGCGGC |
| | | TGACCGGCAAGGGCAAGAAGCGGGTGTTCGCCTCCAACGGCCAGA |
| | | TCACCAACCTGCTGCGGGGCTTCTGGGGCCTGCGGAAGGTGCGGGC |
| | | CGAGAACGACCGGCACCACGCCCTGGACGCCGTGGTGGTGGCCTGC |
| | | TCCACCGTGGCCATGCAGCAGAAGATCACCCGGTTCGTGCGGTACA |
| | | AGGAGATGAACGCCTTCGACGGCAAGACCATCGACAAGGAGACCG |
| | | GCGAGGTGCTGCACCAGAAGACCCACTTCCCCCAGCCCTGGGAGTT |
| | | CTTCGCCCAGGAGGTGATGATCCGGGTGTTCGGCAAGCCCGACGGC |
| | | AAGCCCGAGTTCGAGGAGGCCGACACCCTGGAGAAGCTGCGGACC |
| | | CTGCTGGCCGAGAAGCTGTCCTCCCGGCCCGAGGCCGTGCACGAGT |
| | | ACGTGACCCCCCTGTTCGTGTCCCGGGCCCCCAACCGGAAGATGTC |
| | | CGGCCAGGGCCACATGGAGACCGTGAAGTCCGCCAAGCGGCTGGA |
| | | CGAGGGCGTGTCCGTGCTGCGGGTGCCCCTGACCCAGCTGAAGCTG |
| | | AAGGACCTGGAGAAGATGGTGAACCGGGAGCGGGAGCCCAAGCTG |
| | | TACGAGGCCCTGAAGGCCCGGCTGGAGGCCCACAAGGACGACCCC |
| | | GCCAAGGCCTTCGCCGAGCCCTTCTACAAGTACGACAAGGCCGGCA |
| | | ACCGGACCCAGCAGGTGAAGGCCGTGCGGGTGGAGCAGGTGCAGA |
| | | AGACCGGCGTGTGGGTGCGGAACCACAACGGCATCGCCGACAACG |
| | | CCACCATGGTGCGGGTGGACGTGTTCGAGAAGGGCGACAAGTACTA |
| | | CCTGGTGCCCATCTACTCCTGGCAGGTGGCCAAGGGCATCCTGCCC |
| | | GACCGGGCCGTGGTGCAGGGCAAGGACGAGGAGGACTGGCAGCTG |
| | | ATCGACGACTCCTTCAACTTCAAGTTCTCCCTGCACCCCAACGACCT |
| | | GGTGGAGGTGATCACCAAGAAGGCCCGGATGTTCGGCTACTTCGCC |
| | | TCCTGCCACCGGGGCACCGGCAACATCAACATCCGGATCCACGACC |
| | | TGGACCACAAGATCGGCAAGAACGGCATCCTGGAGGGCATCGGCG |
| | | TGAAGACCGCCCTGTCCTTCCAGAAGTACCAGATCGACGAGCTGGG |
| | | CAAGGAGATCCGGCCCTGCCGGCTGAAGAAGCGGCCCCCCGTGCG |
| | | G |
| 201 | Exemplary | GCAGCATTCAAACCAAACTCAATCAACTACATCCTAGGACTAGACA |
| | coding sequence | TCGGAATCGCATCAGTAGGATGAGCAATGGTAGAAATCGACGAAG |
| | encoding | AAGAAAACCCAATCCGACTAATCGACCTAGGAGTACGAGTATTCGA |
| | Nme1Cas9 HNH | ACGAGCAGAAGTACCAAAAACAGGAGACTCACTAGCAATGGCACG |
| | nickase | ACGACTAGCACGATCAGTACGACGACTAACACGACGACGAGCACA |
| | | CCGACTACTACGAACACGACGACTACTAAAACGAGAAGGAGTACT |
| | | ACAAGCAGCAAACTTCGACGAAAACGGACTAATCAAATCACTACC |
| | | AAACACACCATGACAACTACGAGCAGCAGCACTAGACCGAAAACT |
| | | AACACCACTAGAATGATCAGCAGTACTACTACACCTAATCAAACAC |
| | | CGAGGATACCTATCACAACGAAAAAACGAAGGAGAAACAGCAGAC |
| | | AAAGAACTAGGAGCACTACTAAAAGGAGTAGCAGGAAACGCACAC |
| | | GCACTACAAACAGGAGACTTCCGAACACCAGCAGAACTAGCACTA |
| | | AACAAATTCGAAAAAGAATCAGGACACATCCGAAACCAACGATCA |
| | | GACTACTCACACACATTCTCACGAAAAGACCTACAAGCAGAACTAA |
| | | TCCTACTATTCGAAAAACAAAAAGAATTCGGAAACCCACACGTATC |
| | | AGGAGGACTAAAAGAAGGAATCGAAACACTACTAATGACACAACG |
| | | ACCAGCACTATCAGGAGACGCAGTACAAAAAATGCTAGGACACTG |
| | | CACATTCGAACCAGCAGAACCAAAAGCAGCAAAAAACACATACAC |
| | | AGCAGAACGATTCATCTGACTAACAAAACTAAACAACCTACGAATC |
| | | CTAGAACAAGGATCAGAACGACCACTAACAGACACAGAACGAGCA |
| | | ACACTAATGGACGAACCATACCGAAAATCAAAACTAACATACGCA |
| | | CAAGCACGAAAACTACTAGGACTAGAAGACACAGCATTCTTCAAA |
| | | GGACTACGATACGGAAAAGACAACGCAGAAGCATCAACACTAATG |
| | | GAAATGAAAGCATACCACGCAATCTCACGAGCACTAGAAAAAGAA |
| | | GGACTAAAAGACAAAAAATCACCACTAAACCTATCACCAGAACTA |
| | | CAAGACGAAATCGGAACAGCATTCTCACTATTCAAAACAGACGAA |
| | | GACATCACAGGACGACTAAAAGACCGAATCCAACCAGAAATCCTA |
| | | GAAGCACTACTAAAACACATCTCATTCGACAAATTCGTACAAATCT |
| | | CACTAAAAGCACTACGACGAATCGTACCACTAATGGAACAAGGAA |
| | | AACGATACGACGAAGCATGCGCAGAAATCTACGGAGACCACTACG |
| | | GAAAAAAAAACACAGAAGAAAAAATCTACCTACCACCAATCCCAG |
| | | CAGACGAAATCCGAAACCCAGTAGTACTACGAGCACTATCACAAGC |
| | | ACGAAAAGTAATCAACGGAGTAGTACGACGATACGGATCACCAGC |
| | | ACGAATCCACATCGAAACAGCACGAGAAGTAGGAAAATCATTCAA |
| | | AGACCGAAAAGAAATCGAAAAACGACAAGAAGAAAACCGAAAAG |
| | | ACCGAGAAAAAGCAGCAGCAAAATTCCGAGAATACTTCCCAAACTT |
| | | CGTAGGAGAACCAAAATCAAAAGACATCCTAAAACTACGACTATA |
| | | CGAACAACAACACGGAAAATGCCTATACTCAGGAAAAGAAATCAA |
| | | CCTAGGACGACTAAACGAAAAAGGATACGTAGAAATCGACGCAGC |
| | | ACTACCATTCTCACGAACATGAGACGACTCATTCAACAACAAAGTA |
| | | CTAGTACTAGGATCAGAAAACCAAAACAAAGGAAACCAAACACCA |
| | | TACGAATACTTCAACGGAAAAGACAACTCACGAGAATGACAAGAA |
| | | TTCAAAGCACGAGTAGAAACATCACGATTCCCACGATCAAAAAAAC |
| | | AACGAATCCTACTACAAAAATTCGACGAAGACGGATTCAAAGAAC |
| | | GAAACCTAAACGACACACGATACGTAAACCGATTCCTATGCCAATT |
| | | CGTAGCAGACCGAATGCGACTAACAGGAAAAGGAAAAAAACGAGT |
| | | ATTCGCATCAAACGGACAAATCACAAACCTACTACGAGGATTCTGA |
| | | GGACTACGAAAAGTACGAGCAGAAAACGACCGACACCACGCACTA |
| | | GACGCAGTAGTAGTAGCATGCTCAACAGTAGCAATGCAACAAAAA |
| | | ATCACACGATTCGTACGATACAAAGAAATGAACGCATTCGACGGAA |
| | | AAACAATCGACAAAGAAACAGGAGAAGTACTACACCAAAAAACAC |
| | | ACTTCCCACAACCATGAGAATTCTTCGCACAAGAAGTAATGATCCG |
| | | AGTATTCGGAAAACCAGACGGAAAACCAGAATTCGAAGAAGCAGA |
| | | CACACTAGAAAAACTACGAACACTACTAGCAGAAAAACTATCATCA |
| | | CGACCAGAAGCAGTACACGAATACGTAACACCACTATTCGTATCAC |
| | | GAGCACCAAACCGAAAAATGTCAGGACAAGGACACATGGAAACAG |
| | | TAAAATCAGCAAAACGACTAGACGAAGGAGTATCAGTACTACGAG |
| | | TACCACTAACACAACTAAAACTAAAAGACCTAGAAAAAATGGTAA |
| | | ACCGAGAACGAGAACCAAAACTATACGAAGCACTAAAAGCACGAC |
| | | TAGAAGCACACAAAGACGACCCAGCAAAAGCATTCGCAGAACCAT |
| | | TCTACAAATACGACAAAGCAGGAAACCGAACACAACAAGTAAAAG |
| | | CAGTACGAGTAGAACAAGTACAAAAAACAGGAGTATGAGTACGAA |
| | | ACCACAACGGAATCGCAGACAACGCAACAATGGTACGAGTAGACG |
| | | TATTCGAAAAAGGAGACAAATACTACCTAGTACCAATCTACTCATG |
| | | ACAAGTAGCAAAAGGAATCCTACCAGACCGAGCAGTAGTACAAGG |
| | | AAAAGACGAAGAAGACTGACAACTAATCGACGACTCATTCAACTTC |
| | | AAATTCTCACTACACCCAAACGACCTAGTAGAAGTAATCACAAAAA |
| | | AAGCACGAATGTTCGGATACTTCGCATCATGCCACCGAGGAACAGG |
| | | AAACATCAACATCCGAATCCACGACCTAGACCACAAAATCGGAAA |
| | | AAACGGAATCCTAGAAGGAATCGGAGTAAAAACAGCACTATCATT |
| | | CCAAAAATACCAAATCGACGAACTAGGAAAAGAAATCCGACCATG |
| | | CCGACTAAAAAAACGACCACCAGTACGA |
| 202 | Exemplary open | ATGGCTGCTTTTAAGCCTAATTCTATTAATTATATTCTTGGTCTTGAT |
| | reading frame for | ATTGGTATTGCTTCTGTTGGTTGGGCTATGGTTGAGATTGATGAGGA |
| | Nme1Cas9 HNH | GGAGAATCCTATTCGTCTTATTGATCTTGGTGTTCGTGTTTTTGAGC |
| | nickase | GTGCTGAGGTTCCTAAGACTGGTGATTCTCTTGCTATGGCTCGTCGT |
| | | CTTGCTCGTTCTGTTCGTCGTCTTACTCGTCGTCGTGCTCATCGTCTT |
| | | CTTCGTACTCGTCGTCTTCTTAAGCGTGAGGGTGTTCTTCAGGCTGC |
| | | TAATTTTGATGAGAATGGTCTTATTAAGTCTCTTCCTAATACTCCTT |
| | | GGCAGCTTCGTGCTGCTGCTCTTGATCGTAAGCTTACTCCTCTTGAG |
| | | TGGTCTGCTGTTCTTCTTCATCTTATTAAGCATCGTGGTTATCTTTCT |
| | | CAGCGTAAGAATGAGGGTGAGACTGCTGATAAGGAGCTTGGTGCTC |
| | | TTCTTAAGGGTGTTGCTGGTAATGCTCATGCTCTTCAGACTGGTGAT |
| | | TTTCGTACTCCTGCTGAGCTTGCTCTTAATAAGTTTGAGAAGGAGTC |
| | | TGGTCATATTCGTAATCAGCGTTCTGATTATTCTCATACTTTTTCTCG |
| | | TAAGGATCTTCAGGCTGAGCTTATTCTTCTTTTTGAGAAGCAGAAGG |
| | | AGTTTGGTAATCCTCATGTTTCTGGTGGTCTTAAGGAGGGTATTGAG |
| | | ACTCTTCTTATGACTCAGCGTCCTGCTCTTTCTGGTGATGCTGTTCA |
| | | GAAGATGCTTGGTCATTGTACTTTTGAGCCTGCTGAGCCTAAGGCTG |
| | | CTAAGAATACTTATACTGCTGAGCGTTTTATTTGGCTTACTAAGCTT |
| | | AATAATCTTCGTATTCTTGAGCAGGGTTCTGAGCGTCCTCTTACTGA |
| | | TACTGAGCGTGCTACTCTTATGGATGAGCCTTATCGTAAGTCTAAGC |
| | | TTACTTATGCTCAGGCTCGTAAGCTTCTTGGTCTTGAGGATACTGCT |
| | | TTTTTTAAGGGTCTTCGTTATGGTAAGGATAATGCTGAGGCTTCTAC |
| | | TCTTATGGAGATGAAGGCTTATCATGCTATTTCTCGTGCTCTTGAGA |
| | | AGGAGGGTCTTAAGGATAAGAAGTCTCCTCTTAATCTTTCTCCTGAG |
| | | CTTCAGGATGAGATTGGTACTGCTTTTTCTCTTTTTAAGACTGATGA |
| | | GGATATTACTGGTCGTCTTAAGGATCGTATTCAGCCTGAGATTCTTG |
| | | AGGCTCTTCTTAAGCATATTTCTTTTGATAAGTTTGTTCAGATTTCTC |
| | | TTAAGGCTCTTCGTCGTATTGTTCCTCTTATGGAGCAGGGTAAGCGT |
| | | TATGATGAGGCTTGTGCTGAGATTTATGGTGATCATTATGGTAAGA |
| | | AGAATACTGAGGAGAAGATTTATCTTCCTCCTATTCCTGCTGATGAG |
| | | ATTCGTAATCCTGTTGTTCTTCGTGCTCTTTCTCAGGCTCGTAAGGTT |
| | | ATTAATGGTGTTGTTCGTCGTTATGGTTCTCCTGCTCGTATTCATATT |
| | | GAGACTGCTCGTGAGGTTGGTAAGTCTTTTAAGGATCGTAAGGAGA |
| | | TTGAGAAGCGTCAGGAGGAGAATCGTAAGGATCGTGAGAAGGCTG |
| | | CTGCTAAGTTTCGTGAGTATTTTCCTAATTTTGTTGGTGAGCCTAAG |
| | | TCTAAGGATATTCTTAAGCTTCGTCTTTATGAGCAGCAGCATGGTAA |
| | | GTGTCTTTATTCTGGTAAGGAGATTAATCTTGGTCGTCTTAATGAGA |
| | | AGGGTTATGTTGAGATTGATGCTGCTCTTCCTTTTTCTCGTACTTGG |
| | | GATGATTCTTTTAATAATAAGGTTCTTGTTCTTGGTTCTGAGAATCA |
| | | GAATAAGGGTAATCAGACTCCTTATGAGTATTTTAATGGTAAGGAT |
| | | AATTCTCGTGAGTGGCAGGAGTTTAAGGCTCGTGTTGAGACTTCTC |
| | | GTTTTCCTCGTTCTAAGAAGCAGCGTATTCTTCTTCAGAAGTTTGAT |
| | | GAGGATGGTTTTAAGGAGCGTAATCTTAATGATACTCGTTATGTTA |
| | | ATCGTTTTCTTTGTCAGTTTGTTGCTGATCGTATGCGTCTTACTGGTA |
| | | AGGGTAAGAAGCGTGTTTTTGCTTCTAATGGTCAGATTACTAATCTT |
| | | CTTCGTGGTTTTTGGGGTCTTCGTAAGGTTCGTGCTGAGAATGATCG |
| | | TCATCATGCTCTTGATGCTGTTGTTGTTGCTTGTTCTACTGTTGCTAT |
| | | GCAGCAGAAGATTACTCGTTTTGTTCGTTATAAGGAGATGAATGCT |
| | | TTTGATGGTAAGACTATTGATAAGGAGACTGGTGAGGTTCTTCATC |
| | | AGAAGACTCATTTTCCTCAGCCTTGGGAGTTTTTTGCTCAGGAGGTT |
| | | ATGATTCGTGTTTTTGGTAAGCCTGATGGTAAGCCTGAGTTTGAGGA |
| | | GGCTGATACTCTTGAGAAGCTTCGTACTCTTCTTGCTGAGAAGCTTT |
| | | CTTCTCGTCCTGAGGCTGTTCATGAGTATGTTACTCCTCTTTTTGTTT |
| | | CTCGTGCTCCTAATCGTAAGATGTCTGGTCAGGGTCATATGGAGAC |
| | | TGTTAAGTCTGCTAAGCGTCTTGATGAGGGTGTTTCTGTTCTTCGTG |
| | | TTCCTCTTACTCAGCTTAAGCTTAAGGATCTTGAGAAGATGGTTAAT |
| | | CGTGAGCGTGAGCCTAAGCTTTATGAGGCTCTTAAGGCTCGTCTTG |
| | | AGGCTCATAAGGATGATCCTGCTAAGGCTTTTGCTGAGCCTTTTTAT |
| | | AAGTATGATAAGGCTGGTAATCGTACTCAGCAGGTTAAGGCTGTTC |
| | | GTGTTGAGCAGGTTCAGAAGACTGGTGTTTGGGTTCGTAATCATAA |
| | | TGGTATTGCTGATAATGCTACTATGGTTCGTGTTGATGTTTTTGAGA |
| | | AGGGTGATAAGTATTATCTTGTTCCTATTTATTCTTGGCAGGTTGCT |
| | | AAGGGTATTCTTCCTGATCGTGCTGTTGTTCAGGGTAAGGATGAGG |
| | | AGGATTGGCAGCTTATTGATGATTCTTTTAATTTTAAGTTTTCTCTTC |
| | | ATCCTAATGATCTTGTTGAGGTTATTACTAAGAAGGCTCGTATGTTT |
| | | GGTTATTTTGCTTCTTGTCATCGTGGTACTGGTAATATTAATATTCGT |
| | | ATTCATGATCTTGATCATAAGATTGGTAAGAATGGTATTCTTGAGG |
| | | GTATTGGTGTTAAGACTGCTCTTTCTTTTCAGAAGTATCAGATTGAT |
| | | GAGCTTGGTAAGGAGATTCGTCCTTGTCGTCTTAAGAAGCGTCCTCC |
| | | TGTTCGTUGA |
| 203 | Exemplary open | ATGGCCGCCTTCAAGCCCAACTCCATCAACTACATCCTGGGCCTGG |
| | reading frame for | ACATCGGCATCGCCTCCGTGGGCTGGGCCATGGTGGAGATCGACGA |
| | Nme1Cas9 HNH | GGAGGAGAACCCCATCCGGCTGATCGACCTGGGCGTGCGGGTGTTC |
| | nickase | GAGCGGGCCGAGGTGCCCAAGACCGGCGACTCCCTGGCCATGGCCC |
| | | GGCGGCTGGCCCGGTCCGTGCGGCGGCTGACCCGGCGGCGGGCCCA |
| | | CCGGCTGCTGCGGACCCGGCGGCTGCTGAAGCGGGAGGGCGTGCTG |
| | | CAGGCCGCCAACTTCGACGAGAACGGCCTGATCAAGTCCCTGCCCA |
| | | ACACCCCCTGGCAGCTGCGGGCCGCCGCCCTGGACCGGAAGCTGAC |
| | | CCCCCTGGAGTGGTCCGCCGTGCTGCTGCACCTGATCAAGCACCGG |
| | | GGCTACCTGTCCCAGCGGAAGAACGAGGGCGAGACCGCCGACAAG |
| | | GAGCTGGGCGCCCTGCTGAAGGGCGTGGCCGGCAACGCCCACGCCC |
| | | TGCAGACCGGCGACTTCCGGACCCCCGCCGAGCTGGCCCTGAACAA |
| | | GTTCGAGAAGGAGTCCGGCCACATCCGGAACCAGCGGTCCGACTAC |
| | | TCCCACACCTTCTCCCGGAAGGACCTGCAGGCCGAGCTGATCCTGC |
| | | TGTTCGAGAAGCAGAAGGAGTTCGGCAACCCCCACGTGTCCGGCGG |
| | | CCTGAAGGAGGGCATCGAGACCCTGCTGATGACCCAGCGGCCCGCC |
| | | CTGTCCGGCGACGCCGTGCAGAAGATGCTGGGCCACTGCACCTTCG |
| | | AGCCCGCCGAGCCCAAGGCCGCCAAGAACACCTACACCGCCGAGC |
| | | GGTTCATCTGGCTGACCAAGCTGAACAACCTGCGGATCCTGGAGCA |
| | | GGGCTCCGAGCGGCCCCTGACCGACACCGAGCGGGCCACCCTGATG |
| | | GACGAGCCCTACCGGAAGTCCAAGCTGACCTACGCCCAGGCCCGGA |
| | | AGCTGCTGGGCCTGGAGGACACCGCCTTCTTCAAGGGCCTGCGGTA |
| | | CGGCAAGGACAACGCCGAGGCCTCCACCCTGATGGAGATGAAGGC |
| | | CTACCACGCCATCTCCCGGGCCCTGGAGAAGGAGGGCCTGAAGGAC |
| | | AAGAAGTCCCCCCTGAACCTGTCCCCCGAGCTGCAGGACGAGATCG |
| | | GCACCGCCTTCTCCCTGTTCAAGACCGACGAGGACATCACCGGCCG |
| | | GCTGAAGGACCGGATCCAGCCCGAGATCCTGGAGGCCCTGCTGAAG |
| | | CACATCTCCTTCGACAAGTTCGTGCAGATCTCCCTGAAGGCCCTGCG |
| | | GCGGATCGTGCCCCTGATGGAGCAGGGCAAGCGGTACGACGAGGC |
| | | CTGCGCCGAGATCTACGGCGACCACTACGGCAAGAAGAACACCGA |
| | | GGAGAAGATCTACCTGCCCCCCATCCCCGCCGACGAGATCCGGAAC |
| | | CCCGTGGTGCTGCGGGCCCTGTCCCAGGCCCGGAAGGTGATCAACG |
| | | GCGTGGTGCGGCGGTACGGCTCCCCCGCCCGGATCCACATCGAGAC |
| | | CGCCCGGGAGGTGGGCAAGTCCTTCAAGGACCGGAAGGAGATCGA |
| | | GAAGCGGCAGGAGGAGAACCGGAAGGACCGGGAGAAGGCCGCCG |
| | | CCAAGTTCCGGGAGTACTTCCCCAACTTCGTGGGCGAGCCCAAGTC |
| | | CAAGGACATCCTGAAGCTGCGGCTGTACGAGCAGCAGCACGGCAA |
| | | GTGCCTGTACTCCGGCAAGGAGATCAACCTGGGCCGGCTGAACGAG |
| | | AAGGGCTACGTGGAGATCGACGCCGCCCTGCCCTTCTCCCGGACCT |
| | | GGGACGACTCCTTCAACAACAAGGTGCTGGTGCTGGGCTCCGAGAA |
| | | CCAGAACAAGGGCAACCAGACCCCCTACGAGTACTTCAACGGCAA |
| | | GGACAACTCCCGGGAGTGGCAGGAGTTCAAGGCCCGGGTGGAGAC |
| | | CTCCCGGTTCCCCCGGTCCAAGAAGCAGCGGATCCTGCTGCAGAAG |
| | | TTCGACGAGGACGGCTTCAAGGAGCGGAACCTGAACGACACCCGG |
| | | TACGTGAACCGGTTCCTGTGCCAGTTCGTGGCCGACCGGATGCGGC |
| | | TGACCGGCAAGGGCAAGAAGCGGGTGTTCGCCTCCAACGGCCAGA |
| | | TCACCAACCTGCTGCGGGGCTTCTGGGGCCTGCGGAAGGTGCGGGC |
| | | CGAGAACGACCGGCACCACGCCCTGGACGCCGTGGTGGTGGCCTGC |
| | | TCCACCGTGGCCATGCAGCAGAAGATCACCCGGTTCGTGCGGTACA |
| | | AGGAGATGAACGCCTTCGACGGCAAGACCATCGACAAGGAGACCG |
| | | GCGAGGTGCTGCACCAGAAGACCCACTTCCCCCAGCCCTGGGAGTT |
| | | CTTCGCCCAGGAGGTGATGATCCGGGTGTTCGGCAAGCCCGACGGC |
| | | AAGCCCGAGTTCGAGGAGGCCGACACCCTGGAGAAGCTGCGGACC |
| | | CTGCTGGCCGAGAAGCTGTCCTCCCGGCCCGAGGCCGTGCACGAGT |
| | | ACGTGACCCCCCTGTTCGTGTCCCGGGCCCCCAACCGGAAGATGTC |
| | | CGGCCAGGGCCACATGGAGACCGTGAAGTCCGCCAAGCGGCTGGA |
| | | CGAGGGCGTGTCCGTGCTGCGGGTGCCCCTGACCCAGCTGAAGCTG |
| | | AAGGACCTGGAGAAGATGGTGAACCGGGAGCGGGAGCCCAAGCTG |
| | | TACGAGGCCCTGAAGGCCCGGCTGGAGGCCCACAAGGACGACCCC |
| | | GCCAAGGCCTTCGCCGAGCCCTTCTACAAGTACGACAAGGCCGGCA |
| | | ACCGGACCCAGCAGGTGAAGGCCGTGCGGGTGGAGCAGGTGCAGA |
| | | AGACCGGCGTGTGGGTGCGGAACCACAACGGCATCGCCGACAACG |
| | | CCACCATGGTGCGGGTGGACGTGTTCGAGAAGGGCGACAAGTACTA |
| | | CCTGGTGCCCATCTACTCCTGGCAGGTGGCCAAGGGCATCCTGCCC |
| | | GACCGGGCCGTGGTGCAGGGCAAGGACGAGGAGGACTGGCAGCTG |
| | | ATCGACGACTCCTTCAACTTCAAGTTCTCCCTGCACCCCAACGACCT |
| | | GGTGGAGGTGATCACCAAGAAGGCCCGGATGTTCGGCTACTTCGCC |
| | | TCCTGCCACCGGGGCACCGGCAACATCAACATCCGGATCCACGACC |
| | | TGGACCACAAGATCGGCAAGAACGGCATCCTGGAGGGCATCGGCG |
| | | TGAAGACCGCCCTGTCCTTCCAGAAGTACCAGATCGACGAGCTGGG |
| | | CAAGGAGATCCGGCCCTGCCGGCTGAAGAAGCGGCCCCCCGTGCG |
| | | GUGA |
| 204 | Exemplary open | ATGGCAGCATTCAAACCAAACTCAATCAACTACATCCTAGGACTAG |
| | reading frame for | ACATCGGAATCGCATCAGTAGGATGAGCAATGGTAGAAATCGACG |
| | Nme1Cas9 HNH | AAGAAGAAAACCCAATCCGACTAATCGACCTAGGAGTACGAGTATT |
| | nickase | CGAACGAGCAGAAGTACCAAAAACAGGAGACTCACTAGCAATGGC |
| | | ACGACGACTAGCACGATCAGTACGACGACTAACACGACGACGAGC |
| | | ACACCGACTACTACGAACACGACGACTACTAAAACGAGAAGGAGT |
| | | ACTACAAGCAGCAAACTTCGACGAAAACGGACTAATCAAATCACTA |
| | | CCAAACACACCATGACAACTACGAGCAGCAGCACTAGACCGAAAA |
| | | CTAACACCACTAGAATGATCAGCAGTACTACTACACCTAATCAAAC |
| | | ACCGAGGATACCTATCACAACGAAAAAACGAAGGAGAAACAGCAG |
| | | ACAAAGAACTAGGAGCACTACTAAAAGGAGTAGCAGGAAACGCAC |
| | | ACGCACTACAAACAGGAGACTTCCGAACACCAGCAGAACTAGCAC |
| | | TAAACAAATTCGAAAAAGAATCAGGACACATCCGAAACCAACGAT |
| | | CAGACTACTCACACACATTCTCACGAAAAGACCTACAAGCAGAACT |
| | | AATCCTACTATTCGAAAAACAAAAAGAATTCGGAAACCCACACGTA |
| | | TCAGGAGGACTAAAAGAAGGAATCGAAACACTACTAATGACACAA |
| | | CGACCAGCACTATCAGGAGACGCAGTACAAAAAATGCTAGGACAC |
| | | TGCACATTCGAACCAGCAGAACCAAAAGCAGCAAAAAACACATAC |
| | | ACAGCAGAACGATTCATCTGACTAACAAAACTAAACAACCTACGAA |
| | | TCCTAGAACAAGGATCAGAACGACCACTAACAGACACAGAACGAG |
| | | CAACACTAATGGACGAACCATACCGAAAATCAAAACTAACATACG |
| | | CACAAGCACGAAAACTACTAGGACTAGAAGACACAGCATTCTTCAA |
| | | AGGACTACGATACGGAAAAGACAACGCAGAAGCATCAACACTAAT |
| | | GGAAATGAAAGCATACCACGCAATCTCACGAGCACTAGAAAAAGA |
| | | AGGACTAAAAGACAAAAAATCACCACTAAACCTATCACCAGAACT |
| | | ACAAGACGAAATCGGAACAGCATTCTCACTATTCAAAACAGACGA |
| | | AGACATCACAGGACGACTAAAAGACCGAATCCAACCAGAAATCCT |
| | | AGAAGCACTACTAAAACACATCTCATTCGACAAATTCGTACAAATC |
| | | TCACTAAAAGCACTACGACGAATCGTACCACTAATGGAACAAGGA |
| | | AAACGATACGACGAAGCATGCGCAGAAATCTACGGAGACCACTAC |
| | | GGAAAAAAAAACACAGAAGAAAAAATCTACCTACCACCAATCCCA |
| | | GCAGACGAAATCCGAAACCCAGTAGTACTACGAGCACTATCACAA |
| | | GCACGAAAAGTAATCAACGGAGTAGTACGACGATACGGATCACCA |
| | | GCACGAATCCACATCGAAACAGCACGAGAAGTAGGAAAATCATTC |
| | | AAAGACCGAAAAGAAATCGAAAAACGACAAGAAGAAAACCGAAA |
| | | AGACCGAGAAAAAGCAGCAGCAAAATTCCGAGAATACTTCCCAAA |
| | | CTTCGTAGGAGAACCAAAATCAAAAGACATCCTAAAACTACGACTA |
| | | TACGAACAACAACACGGAAAATGCCTATACTCAGGAAAAGAAATC |
| | | AACCTAGGACGACTAAACGAAAAAGGATACGTAGAAATCGACGCA |
| | | GCACTACCATTCTCACGAACATGAGACGACTCATTCAACAACAAAG |
| | | TACTAGTACTAGGATCAGAAAACCAAAACAAAGGAAACCAAACAC |
| | | CATACGAATACTTCAACGGAAAAGACAACTCACGAGAATGACAAG |
| | | AATTCAAAGCACGAGTAGAAACATCACGATTCCCACGATCAAAAA |
| | | AACAACGAATCCTACTACAAAAATTCGACGAAGACGGATTCAAAG |
| | | AACGAAACCTAAACGACACACGATACGTAAACCGATTCCTATGCCA |
| | | ATTCGTAGCAGACCGAATGCGACTAACAGGAAAAGGAAAAAAACG |
| | | AGTATTCGCATCAAACGGACAAATCACAAACCTACTACGAGGATTC |
| | | TGAGGACTACGAAAAGTACGAGCAGAAAACGACCGACACCACGCA |
| | | CTAGACGCAGTAGTAGTAGCATGCTCAACAGTAGCAATGCAACAAA |
| | | AAATCACACGATTCGTACGATACAAAGAAATGAACGCATTCGACGG |
| | | AAAAACAATCGACAAAGAAACAGGAGAAGTACTACACCAAAAAAC |
| | | ACACTTCCCACAACCATGAGAATTCTTCGCACAAGAAGTAATGATC |
| | | CGAGTATTCGGAAAACCAGACGGAAAACCAGAATTCGAAGAAGCA |
| | | GACACACTAGAAAAACTACGAACACTACTAGCAGAAAAACTATCA |
| | | TCACGACCAGAAGCAGTACACGAATACGTAACACCACTATTCGTAT |
| | | CACGAGCACCAAACCGAAAAATGTCAGGACAAGGACACATGGAAA |
| | | CAGTAAAATCAGCAAAACGACTAGACGAAGGAGTATCAGTACTAC |
| | | GAGTACCACTAACACAACTAAAACTAAAAGACCTAGAAAAAATGG |
| | | TAAACCGAGAACGAGAACCAAAACTATACGAAGCACTAAAAGCAC |
| | | GACTAGAAGCACACAAAGACGACCCAGCAAAAGCATTCGCAGAAC |
| | | CATTCTACAAATACGACAAAGCAGGAAACCGAACACAACAAGTAA |
| | | AAGCAGTACGAGTAGAACAAGTACAAAAAACAGGAGTATGAGTAC |
| | | GAAACCACAACGGAATCGCAGACAACGCAACAATGGTACGAGTAG |
| | | ACGTATTCGAAAAAGGAGACAAATACTACCTAGTACCAATCTACTC |
| | | ATGACAAGTAGCAAAAGGAATCCTACCAGACCGAGCAGTAGTACA |
| | | AGGAAAAGACGAAGAAGACTGACAACTAATCGACGACTCATTCAA |
| | | CTTCAAATTCTCACTACACCCAAACGACCTAGTAGAAGTAATCACA |
| | | AAAAAAGCACGAATGTTCGGATACTTCGCATCATGCCACCGAGGAA |
| | | CAGGAAACATCAACATCCGAATCCACGACCTAGACCACAAAATCG |
| | | GAAAAAACGGAATCCTAGAAGGAATCGGAGTAAAAACAGCACTAT |
| | | CATTCCAAAAATACCAAATCGACGAACTAGGAAAAGAAATCCGAC |
| | | CATGCCGACTAAAAAAACGACCACCAGTACGAUAA |
| 205 | Exemplary amino | MAAFKPNPINYILGLDIGIASVGWAMVEIDEEENPIRLIDLGVRVFERAE |
| | acid sequence of | VPKTGDSLAMARRLARSVRRLTRRRAHRLLRARRLLKREGVLQAADF |
| | Nme2Cas9 | DENGLIKSLPNTPWQLRAAALDRKLTPLEWSAVLLHLIKHRGYLSQRK |
| | cleavase | NEGETADKELGALLKGVANNAHALQTGDFRTPAELALNKFEKESGHI |
| | | RNQRGDYSHTFSRKDLQAELILLFEKQKEFGNPHVSGGLKEGIETLLMT |
| | | QRPALSGDAVQKMLGHCTFEPAEPKAAKNTYTAERFIWLTKLNNLRIL |
| | | EQGSERPLTDTERATLMDEPYRKSKLTYAQARKLLGLEDTAFFKGLRY |
| | | GKDNAEASTLMEMKAYHAISRALEKEGLKDKKSPLNLSSELQDEIGTA |
| | | FSLFKTDEDITGRLKDRVQPEILEALLKHISFDKFVQISLKALRRIVPLME |
| | | QGKRYDEACAEIYGDHYGKKNTEEKIYLPPIPADEIRNPVVLRALSQAR |
| | | KVINGVVRRYGSPARIHIETAREVGKSFKDRKEIEKRQEENRKDREKA |
| | | AAKFREYFPNFVGEPKSKDILKLRLYEQQHGKCLYSGKEINLVRLNEK |
| | | GYVEIDHALPFSRTWDDSFNNKVLVLGSENQNKGNQTPYEYFNGKDN |
| | | SREWQEFKARVETSRFPRSKKQRILLQKFDEDGFKECNLNDTRYVNRF |
| | | LCQFVADHILLTGKGKRRVFASNGQITNLLRGFWGLRKVRAENDRHH |
| | | ALDAVVVACSTVAMQQKITRFVRYKEMNAFDGKTIDKETGKVLHQK |
| | | THFPQPWEFFAQEVMIRVFGKPDGKPEFEEADTPEKLRTLLAEKLSSRP |
| | | EAVHEYVTPLFVSRAPNRKMSGAHKDTLRSAKRFVKHNEKISVKRVW |
| | | LTEIKLADLENMVNYKNGREIELYEALKARLEAYGGNAKQAFDPKDN |
| | | PFYKKGGQLVKAVRVEKTQESGVLLNKKNAYTIADNGDMVRVDVFC |
| | | KVDKKGKNQYFIVPIYAWQVAENILPDIDCKGYRIDDSYTFCFSLHKY |
| | | DLIAFQKDEKSKVEFAYYINCDSSNGRFYLAWHDKGSKEQQFRISTQN |
| | | LVLIQKYQVNELGKEIRPCRLKKRPPVR |
| 206 | Exemplary | GCTGCTTTTAAGCCTAATCCTATTAATTATATTCTTGGTCTTGATATT |
| | coding sequence | GGTATTGCTTCTGTTGGTTGGGCTATGGTTGAGATTGATGAGGAGG |
| | encoding | AGAATCCTATTCGTCTTATTGATCTTGGTGTTCGTGTTTTTGAGCGT |
| | Nme2Cas9 | GCTGAGGTTCCTAAGACTGGTGATTCTCTTGCTATGGCTCGTCGTCT |
| | cleavase | TGCTCGTTCTGTTCGTCGTCTTACTCGTCGTCGTGCTCATCGTCTTCT |
| | | TCGTGCTCGTCGTCTTCTTAAGCGTGAGGGTGTTCTTCAGGCTGCTG |
| | | ATTTTGATGAGAATGGTCTTATTAAGTCTCTTCCTAATACTCCTTGG |
| | | CAGCTTCGTGCTGCTGCTCTTGATCGTAAGCTTACTCCTCTTGAGTG |
| | | GTCTGCTGTTCTTCTTCATCTTATTAAGCATCGTGGTTATCTTTCTCA |
| | | GCGTAAGAATGAGGGTGAGACTGCTGATAAGGAGCTTGGTGCTCTT |
| | | CTTAAGGGTGTTGCTAATAATGCTCATGCTCTTCAGACTGGTGATTT |
| | | TCGTACTCCTGCTGAGCTTGCTCTTAATAAGTTTGAGAAGGAGTCTG |
| | | GTCATATTCGTAATCAGCGTGGTGATTATTCTCATACTTTTTCTCGT |
| | | AAGGATCTTCAGGCTGAGCTTATTCTTCTTTTTGAGAAGCAGAAGG |
| | | AGTTTGGTAATCCTCATGTTTCTGGTGGTCTTAAGGAGGGTATTGAG |
| | | ACTCTTCTTATGACTCAGCGTCCTGCTCTTTCTGGTGATGCTGTTCA |
| | | GAAGATGCTTGGTCATTGTACTTTTGAGCCTGCTGAGCCTAAGGCTG |
| | | CTAAGAATACTTATACTGCTGAGCGTTTTATTTGGCTTACTAAGCTT |
| | | AATAATCTTCGTATTCTTGAGCAGGGTTCTGAGCGTCCTCTTACTGA |
| | | TACTGAGCGTGCTACTCTTATGGATGAGCCTTATCGTAAGTCTAAGC |
| | | TTACTTATGCTCAGGCTCGTAAGCTTCTTGGTCTTGAGGATACTGCT |
| | | TTTTTTAAGGGTCTTCGTTATGGTAAGGATAATGCTGAGGCTTCTAC |
| | | TCTTATGGAGATGAAGGCTTATCATGCTATTTCTCGTGCTCTTGAGA |
| | | AGGAGGGTCTTAAGGATAAGAAGTCTCCTCTTAATCTTTCTTCTGAG |
| | | CTTCAGGATGAGATTGGTACTGCTTTTTCTCTTTTTAAGACTGATGA |
| | | GGATATTACTGGTCGTCTTAAGGATCGTGTTCAGCCTGAGATTCTTG |
| | | AGGCTCTTCTTAAGCATATTTCTTTTGATAAGTTTGTTCAGATTTCTC |
| | | TTAAGGCTCTTCGTCGTATTGTTCCTCTTATGGAGCAGGGTAAGCGT |
| | | TATGATGAGGCTTGTGCTGAGATTTATGGTGATCATTATGGTAAGA |
| | | AGAATACTGAGGAGAAGATTTATCTTCCTCCTATTCCTGCTGATGAG |
| | | ATTCGTAATCCTGTTGTTCTTCGTGCTCTTTCTCAGGCTCGTAAGGTT |
| | | ATTAATGGTGTTGTTCGTCGTTATGGTTCTCCTGCTCGTATTCATATT |
| | | GAGACTGCTCGTGAGGTTGGTAAGTCTTTTAAGGATCGTAAGGAGA |
| | | TTGAGAAGCGTCAGGAGGAGAATCGTAAGGATCGTGAGAAGGCTG |
| | | CTGCTAAGTTTCGTGAGTATTTTCCTAATTTTGTTGGTGAGCCTAAG |
| | | TCTAAGGATATTCTTAAGCTTCGTCTTTATGAGCAGCAGCATGGTAA |
| | | GTGTCTTTATTCTGGTAAGGAGATTAATCTTGTTCGTCTTAATGAGA |
| | | AGGGTTATGTTGAGATTGATCATGCTCTTCCTTTTTCTCGTACTTGG |
| | | GATGATTCTTTTAATAATAAGGTTCTTGTTCTTGGTTCTGAGAATCA |
| | | GAATAAGGGTAATCAGACTCCTTATGAGTATTTTAATGGTAAGGAT |
| | | AATTCTCGTGAGTGGCAGGAGTTTAAGGCTCGTGTTGAGACTTCTC |
| | | GTTTTCCTCGTTCTAAGAAGCAGCGTATTCTTCTTCAGAAGTTTGAT |
| | | GAGGATGGTTTTAAGGAGTGTAATCTTAATGATACTCGTTATGTTAA |
| | | TCGTTTTCTTTGTCAGTTTGTTGCTGATCATATTCTTCTTACTGGTAA |
| | | GGGTAAGCGTCGTGTTTTTGCTTCTAATGGTCAGATTACTAATCTTC |
| | | TTCGTGGTTTTTGGGGTCTTCGTAAGGTTCGTGCTGAGAATGATCGT |
| | | CATCATGCTCTTGATGCTGTTGTTGTTGCTTGTTCTACTGTTGCTATG |
| | | CAGCAGAAGATTACTCGTTTTGTTCGTTATAAGGAGATGAATGCTTT |
| | | TGATGGTAAGACTATTGATAAGGAGACTGGTAAGGTTCTTCATCAG |
| | | AAGACTCATTTTCCTCAGCCTTGGGAGTTTTTTGCTCAGGAGGTTAT |
| | | GATTCGTGTTTTTGGTAAGCCTGATGGTAAGCCTGAGTTTGAGGAG |
| | | GCTGATACTCCTGAGAAGCTTCGTACTCTTCTTGCTGAGAAGCTTTC |
| | | TTCTCGTCCTGAGGCTGTTCATGAGTATGTTACTCCTCTTTTTGTTTC |
| | | TCGTGCTCCTAATCGTAAGATGTCTGGTGCTCATAAGGATACTCTTC |
| | | GTTCTGCTAAGCGTTTTGTTAAGCATAATGAGAAGATTTCTGTTAAG |
| | | CGTGTTTGGCTTACTGAGATTAAGCTTGCTGATCTTGAGAATATGGT |
| | | TAATTATAAGAATGGTCGTGAGATTGAGCTTTATGAGGCTCTTAAG |
| | | GCTCGTCTTGAGGCTTATGGTGGTAATGCTAAGCAGGCTTTTGATCC |
| | | TAAGGATAATCCTTTTTATAAGAAGGGTGGTCAGCTTGTTAAGGCT |
| | | GTTCGTGTTGAGAAGACTCAGGAGTCTGGTGTTCTTCTTAATAAGA |
| | | AGAATGCTTATACTATTGCTGATAATGGTGATATGGTTCGTGTTGAT |
| | | GTTTTTTGTAAGGTTGATAAGAAGGGTAAGAATCAGTATTTTATTGT |
| | | TCCTATTTATGCTTGGCAGGTTGCTGAGAATATTCTTCCTGATATTG |
| | | ATTGTAAGGGTTATCGTATTGATGATTCTTATACTTTTTGTTTTTCTC |
| | | TTCATAAGTATGATCTTATTGCTTTTCAGAAGGATGAGAAGTCTAAG |
| | | GTTGAGTTTGCTTATTATATTAATTGTGATTCTTCTAATGGTCGTTTT |
| | | TATCTTGCTTGGCATGATAAGGGTTCTAAGGAGCAGCAGTTTCGTAT |
| | | TTCTACTCAGAATCTTGTTCTTATTCAGAAGTATCAGGTTAATGAGC |
| | | TTGGTAAGGAGATTCGTCCTTGTCGTCTTAAGAAGCGTCCTCCTGTT |
| | | CGT |
| 207 | Exemplary | GCCGCCTTCAAGCCCAACCCCATCAACTACATCCTGGGCCTGGACA |
| | coding sequence | TCGGCATCGCCTCCGTGGGCTGGGCCATGGTGGAGATCGACGAGGA |
| | encoding | GGAGAACCCCATCCGGCTGATCGACCTGGGCGTGCGGGTGTTCGAG |
| | Nme2Cas9 | CGGGCCGAGGTGCCCAAGACCGGCGACTCCCTGGCCATGGCCCGGC |
| | cleavase | GGCTGGCCCGGTCCGTGCGGCGGCTGACCCGGCGGCGGGCCCACCG |
| | | GCTGCTGCGGGCCCGGCGGCTGCTGAAGCGGGAGGGCGTGCTGCA |
| | | GGCCGCCGACTTCGACGAGAACGGCCTGATCAAGTCCCTGCCCAAC |
| | | ACCCCCTGGCAGCTGCGGGCCGCCGCCCTGGACCGGAAGCTGACCC |
| | | CCCTGGAGTGGTCCGCCGTGCTGCTGCACCTGATCAAGCACCGGGG |
| | | CTACCTGTCCCAGCGGAAGAACGAGGGCGAGACCGCCGACAAGGA |
| | | GCTGGGCGCCCTGCTGAAGGGCGTGGCCAACAACGCCCACGCCCTG |
| | | CAGACCGGCGACTTCCGGACCCCCGCCGAGCTGGCCCTGAACAAGT |
| | | TCGAGAAGGAGTCCGGCCACATCCGGAACCAGCGGGGCGACTACT |
| | | CCCACACCTTCTCCCGGAAGGACCTGCAGGCCGAGCTGATCCTGCT |
| | | GTTCGAGAAGCAGAAGGAGTTCGGCAACCCCCACGTGTCCGGCGGC |
| | | CTGAAGGAGGGCATCGAGACCCTGCTGATGACCCAGCGGCCCGCCC |
| | | TGTCCGGCGACGCCGTGCAGAAGATGCTGGGCCACTGCACCTTCGA |
| | | GCCCGCCGAGCCCAAGGCCGCCAAGAACACCTACACCGCCGAGCG |
| | | GTTCATCTGGCTGACCAAGCTGAACAACCTGCGGATCCTGGAGCAG |
| | | GGCTCCGAGCGGCCCCTGACCGACACCGAGCGGGCCACCCTGATGG |
| | | ACGAGCCCTACCGGAAGTCCAAGCTGACCTACGCCCAGGCCCGGAA |
| | | GCTGCTGGGCCTGGAGGACACCGCCTTCTTCAAGGGCCTGCGGTAC |
| | | GGCAAGGACAACGCCGAGGCCTCCACCCTGATGGAGATGAAGGCC |
| | | TACCACGCCATCTCCCGGGCCCTGGAGAAGGAGGGCCTGAAGGAC |
| | | AAGAAGTCCCCCCTGAACCTGTCCTCCGAGCTGCAGGACGAGATCG |
| | | GCACCGCCTTCTCCCTGTTCAAGACCGACGAGGACATCACCGGCCG |
| | | GCTGAAGGACCGGGTGCAGCCCGAGATCCTGGAGGCCCTGCTGAA |
| | | GCACATCTCCTTCGACAAGTTCGTGCAGATCTCCCTGAAGGCCCTGC |
| | | GGCGGATCGTGCCCCTGATGGAGCAGGGCAAGCGGTACGACGAGG |
| | | CCTGCGCCGAGATCTACGGCGACCACTACGGCAAGAAGAACACCG |
| | | AGGAGAAGATCTACCTGCCCCCCATCCCCGCCGACGAGATCCGGAA |
| | | CCCCGTGGTGCTGCGGGCCCTGTCCCAGGCCCGGAAGGTGATCAAC |
| | | GGCGTGGTGCGGCGGTACGGCTCCCCCGCCCGGATCCACATCGAGA |
| | | CCGCCCGGGAGGTGGGCAAGTCCTTCAAGGACCGGAAGGAGATCG |
| | | AGAAGCGGCAGGAGGAGAACCGGAAGGACCGGGAGAAGGCCGCC |
| | | GCCAAGTTCCGGGAGTACTTCCCCAACTTCGTGGGCGAGCCCAAGT |
| | | CCAAGGACATCCTGAAGCTGCGGCTGTACGAGCAGCAGCACGGCA |
| | | AGTGCCTGTACTCCGGCAAGGAGATCAACCTGGTGCGGCTGAACGA |
| | | GAAGGGCTACGTGGAGATCGACCACGCCCTGCCCTTCTCCCGGACC |
| | | TGGGACGACTCCTTCAACAACAAGGTGCTGGTGCTGGGCTCCGAGA |
| | | ACCAGAACAAGGGCAACCAGACCCCCTACGAGTACTTCAACGGCA |
| | | AGGACAACTCCCGGGAGTGGCAGGAGTTCAAGGCCCGGGTGGAGA |
| | | CCTCCCGGTTCCCCCGGTCCAAGAAGCAGCGGATCCTGCTGCAGAA |
| | | GTTCGACGAGGACGGCTTCAAGGAGTGCAACCTGAACGACACCCG |
| | | GTACGTGAACCGGTTCCTGTGCCAGTTCGTGGCCGACCACATCCTG |
| | | CTGACCGGCAAGGGCAAGCGGCGGGTGTTCGCCTCCAACGGCCAG |
| | | ATCACCAACCTGCTGCGGGGCTTCTGGGGCCTGCGGAAGGTGCGGG |
| | | CCGAGAACGACCGGCACCACGCCCTGGACGCCGTGGTGGTGGCCTG |
| | | CTCCACCGTGGCCATGCAGCAGAAGATCACCCGGTTCGTGCGGTAC |
| | | AAGGAGATGAACGCCTTCGACGGCAAGACCATCGACAAGGAGACC |
| | | GGCAAGGTGCTGCACCAGAAGACCCACTTCCCCCAGCCCTGGGAGT |
| | | TCTTCGCCCAGGAGGTGATGATCCGGGTGTTCGGCAAGCCCGACGG |
| | | CAAGCCCGAGTTCGAGGAGGCCGACACCCCCGAGAAGCTGCGGAC |
| | | CCTGCTGGCCGAGAAGCTGTCCTCCCGGCCCGAGGCCGTGCACGAG |
| | | TACGTGACCCCCCTGTTCGTGTCCCGGGCCCCCAACCGGAAGATGT |
| | | CCGGCGCCCACAAGGACACCCTGCGGTCCGCCAAGCGGTTCGTGAA |
| | | GCACAACGAGAAGATCTCCGTGAAGCGGGTGTGGCTGACCGAGAT |
| | | CAAGCTGGCCGACCTGGAGAACATGGTGAACTACAAGAACGGCCG |
| | | GGAGATCGAGCTGTACGAGGCCCTGAAGGCCCGGCTGGAGGCCTA |
| | | CGGCGGCAACGCCAAGCAGGCCTTCGACCCCAAGGACAACCCCTTC |
| | | TACAAGAAGGGCGGCCAGCTGGTGAAGGCCGTGCGGGTGGAGAAG |
| | | ACCCAGGAGTCCGGCGTGCTGCTGAACAAGAAGAACGCCTACACC |
| | | ATCGCCGACAACGGCGACATGGTGCGGGTGGACGTGTTCTGCAAGG |
| | | TGGACAAGAAGGGCAAGAACCAGTACTTCATCGTGCCCATCTACGC |
| | | CTGGCAGGTGGCCGAGAACATCCTGCCCGACATCGACTGCAAGGGC |
| | | TACCGGATCGACGACTCCTACACCTTCTGCTTCTCCCTGCACAAGTA |
| | | CGACCTGATCGCCTTCCAGAAGGACGAGAAGTCCAAGGTGGAGTTC |
| | | GCCTACTACATCAACTGCGACTCCTCCAACGGCCGGTTCTACCTGGC |
| | | CTGGCACGACAAGGGCTCCAAGGAGCAGCAGTTCCGGATCTCCACC |
| | | CAGAACCTGGTGCTGATCCAGAAGTACCAGGTGAACGAGCTGGGC |
| | | AAGGAGATCCGGCCCTGCCGGCTGAAGAAGCGGCCCCCCGTGCGG |
| 208 | Exemplary | GCAGCATTCAAACCAAACCCAATCAACTACATCCTAGGACTAGACA |
| | coding sequence | TCGGAATCGCATCAGTAGGATGAGCAATGGTAGAAATCGACGAAG |
| | encoding | AAGAAAACCCAATCCGACTAATCGACCTAGGAGTACGAGTATTCGA |
| | Nme2Cas9 | ACGAGCAGAAGTACCAAAAACAGGAGACTCACTAGCAATGGCACG |
| | cleavase | ACGACTAGCACGATCAGTACGACGACTAACACGACGACGAGCACA |
| | | CCGACTACTACGAGCACGACGACTACTAAAACGAGAAGGAGTACT |
| | | ACAAGCAGCAGACTTCGACGAAAACGGACTAATCAAATCACTACC |
| | | AAACACACCATGACAACTACGAGCAGCAGCACTAGACCGAAAACT |
| | | AACACCACTAGAATGATCAGCAGTACTACTACACCTAATCAAACAC |
| | | CGAGGATACCTATCACAACGAAAAAACGAAGGAGAAACAGCAGAC |
| | | AAAGAACTAGGAGCACTACTAAAAGGAGTAGCAAACAACGCACAC |
| | | GCACTACAAACAGGAGACTTCCGAACACCAGCAGAACTAGCACTA |
| | | AACAAATTCGAAAAAGAATCAGGACACATCCGAAACCAACGAGGA |
| | | GACTACTCACACACATTCTCACGAAAAGACCTACAAGCAGAACTAA |
| | | TCCTACTATTCGAAAAACAAAAAGAATTCGGAAACCCACACGTATC |
| | | AGGAGGACTAAAAGAAGGAATCGAAACACTACTAATGACACAACG |
| | | ACCAGCACTATCAGGAGACGCAGTACAAAAAATGCTAGGACACTG |
| | | CACATTCGAACCAGCAGAACCAAAAGCAGCAAAAAACACATACAC |
| | | AGCAGAACGATTCATCTGACTAACAAAACTAAACAACCTACGAATC |
| | | CTAGAACAAGGATCAGAACGACCACTAACAGACACAGAACGAGCA |
| | | ACACTAATGGACGAACCATACCGAAAATCAAAACTAACATACGCA |
| | | CAAGCACGAAAACTACTAGGACTAGAAGACACAGCATTCTTCAAA |
| | | GGACTACGATACGGAAAAGACAACGCAGAAGCATCAACACTAATG |
| | | GAAATGAAAGCATACCACGCAATCTCACGAGCACTAGAAAAAGAA |
| | | GGACTAAAAGACAAAAAATCACCACTAAACCTATCATCAGAACTAC |
| | | AAGACGAAATCGGAACAGCATTCTCACTATTCAAAACAGACGAAG |
| | | ACATCACAGGACGACTAAAAGACCGAGTACAACCAGAAATCCTAG |
| | | AAGCACTACTAAAACACATCTCATTCGACAAATTCGTACAAATCTC |
| | | ACTAAAAGCACTACGACGAATCGTACCACTAATGGAACAAGGAAA |
| | | ACGATACGACGAAGCATGCGCAGAAATCTACGGAGACCACTACGG |
| | | AAAAAAAAACACAGAAGAAAAAATCTACCTACCACCAATCCCAGC |
| | | AGACGAAATCCGAAACCCAGTAGTACTACGAGCACTATCACAAGC |
| | | ACGAAAAGTAATCAACGGAGTAGTACGACGATACGGATCACCAGC |
| | | ACGAATCCACATCGAAACAGCACGAGAAGTAGGAAAATCATTCAA |
| | | AGACCGAAAAGAAATCGAAAAACGACAAGAAGAAAACCGAAAAG |
| | | ACCGAGAAAAAGCAGCAGCAAAATTCCGAGAATACTTCCCAAACTT |
| | | CGTAGGAGAACCAAAATCAAAAGACATCCTAAAACTACGACTATA |
| | | CGAACAACAACACGGAAAATGCCTATACTCAGGAAAAGAAATCAA |
| | | CCTAGTACGACTAAACGAAAAAGGATACGTAGAAATCGACCACGC |
| | | ACTACCATTCTCACGAACATGAGACGACTCATTCAACAACAAAGTA |
| | | CTAGTACTAGGATCAGAAAACCAAAACAAAGGAAACCAAACACCA |
| | | TACGAATACTTCAACGGAAAAGACAACTCACGAGAATGACAAGAA |
| | | TTCAAAGCACGAGTAGAAACATCACGATTCCCACGATCAAAAAAAC |
| | | AACGAATCCTACTACAAAAATTCGACGAAGACGGATTCAAAGAAT |
| | | GCAACCTAAACGACACACGATACGTAAACCGATTCCTATGCCAATT |
| | | CGTAGCAGACCACATCCTACTAACAGGAAAAGGAAAACGACGAGT |
| | | ATTCGCATCAAACGGACAAATCACAAACCTACTACGAGGATTCTGA |
| | | GGACTACGAAAAGTACGAGCAGAAAACGACCGACACCACGCACTA |
| | | GACGCAGTAGTAGTAGCATGCTCAACAGTAGCAATGCAACAAAAA |
| | | ATCACACGATTCGTACGATACAAAGAAATGAACGCATTCGACGGAA |
| | | AAACAATCGACAAAGAAACAGGAAAAGTACTACACCAAAAAACAC |
| | | ACTTCCCACAACCATGAGAATTCTTCGCACAAGAAGTAATGATCCG |
| | | AGTATTCGGAAAACCAGACGGAAAACCAGAATTCGAAGAAGCAGA |
| | | CACACCAGAAAAACTACGAACACTACTAGCAGAAAAACTATCATC |
| | | ACGACCAGAAGCAGTACACGAATACGTAACACCACTATTCGTATCA |
| | | CGAGCACCAAACCGAAAAATGTCAGGAGCACACAAAGACACACTA |
| | | CGATCAGCAAAACGATTCGTAAAACACAACGAAAAAATCTCAGTA |
| | | AAACGAGTATGACTAACAGAAATCAAACTAGCAGACCTAGAAAAC |
| | | ATGGTAAACTACAAAAACGGACGAGAAATCGAACTATACGAAGCA |
| | | CTAAAAGCACGACTAGAAGCATACGGAGGAAACGCAAAACAAGCA |
| | | TTCGACCCAAAAGACAACCCATTCTACAAAAAAGGAGGACAACTA |
| | | GTAAAAGCAGTACGAGTAGAAAAAACACAAGAATCAGGAGTACTA |
| | | CTAAACAAAAAAAACGCATACACAATCGCAGACAACGGAGACATG |
| | | GTACGAGTAGACGTATTCTGCAAAGTAGACAAAAAAGGAAAAAAC |
| | | CAATACTTCATCGTACCAATCTACGCATGACAAGTAGCAGAAAACA |
| | | TCCTACCAGACATCGACTGCAAAGGATACCGAATCGACGACTCATA |
| | | CACATTCTGCTTCTCACTACACAAATACGACCTAATCGCATTCCAAA |
| | | AAGACGAAAAATCAAAAGTAGAATTCGCATACTACATCAACTGCG |
| | | ACTCATCAAACGGACGATTCTACCTAGCATGACACGACAAAGGATC |
| | | AAAAGAACAACAATTCCGAATCTCAACACAAAACCTAGTACTAATC |
| | | CAAAAATACCAAGTAAACGAACTAGGAAAAGAAATCCGACCATGC |
| | | CGACTAAAAAAACGACCACCAGTACGA |
| 209 | Exemplary open | ATGGCTGCTTTTAAGCCTAATCCTATTAATTATATTCTTGGTCTTGAT |
| | reading frame for | ATTGGTATTGCTTCTGTTGGTTGGGCTATGGTTGAGATTGATGAGGA |
| | Nme2Cas9 | GGAGAATCCTATTCGTCTTATTGATCTTGGTGTTCGTGTTTTTGAGC |
| | cleavase | GTGCTGAGGTTCCTAAGACTGGTGATTCTCTTGCTATGGCTCGTCGT |
| | | CTTGCTCGTTCTGTTCGTCGTCTTACTCGTCGTCGTGCTCATCGTCTT |
| | | CTTCGTGCTCGTCGTCTTCTTAAGCGTGAGGGTGTTCTTCAGGCTGC |
| | | TGATTTTGATGAGAATGGTCTTATTAAGTCTCTTCCTAATACTCCTT |
| | | GGCAGCTTCGTGCTGCTGCTCTTGATCGTAAGCTTACTCCTCTTGAG |
| | | TGGTCTGCTGTTCTTCTTCATCTTATTAAGCATCGTGGTTATCTTTCT |
| | | CAGCGTAAGAATGAGGGTGAGACTGCTGATAAGGAGCTTGGTGCTC |
| | | TTCTTAAGGGTGTTGCTAATAATGCTCATGCTCTTCAGACTGGTGAT |
| | | TTTCGTACTCCTGCTGAGCTTGCTCTTAATAAGTTTGAGAAGGAGTC |
| | | TGGTCATATTCGTAATCAGCGTGGTGATTATTCTCATACTTTTTCTC |
| | | GTAAGGATCTTCAGGCTGAGCTTATTCTTCTTTTTGAGAAGCAGAAG |
| | | GAGTTTGGTAATCCTCATGTTTCTGGTGGTCTTAAGGAGGGTATTGA |
| | | GACTCTTCTTATGACTCAGCGTCCTGCTCTTTCTGGTGATGCTGTTC |
| | | AGAAGATGCTTGGTCATTGTACTTTTGAGCCTGCTGAGCCTAAGGCT |
| | | GCTAAGAATACTTATACTGCTGAGCGTTTTATTTGGCTTACTAAGCT |
| | | TAATAATCTTCGTATTCTTGAGCAGGGTTCTGAGCGTCCTCTTACTG |
| | | ATACTGAGCGTGCTACTCTTATGGATGAGCCTTATCGTAAGTCTAAG |
| | | CTTACTTATGCTCAGGCTCGTAAGCTTCTTGGTCTTGAGGATACTGC |
| | | TTTTTTTAAGGGTCTTCGTTATGGTAAGGATAATGCTGAGGCTTCTA |
| | | CTCTTATGGAGATGAAGGCTTATCATGCTATTTCTCGTGCTCTTGAG |
| | | AAGGAGGGTCTTAAGGATAAGAAGTCTCCTCTTAATCTTTCTTCTGA |
| | | GCTTCAGGATGAGATTGGTACTGCTTTTTCTCTTTTTAAGACTGATG |
| | | AGGATATTACTGGTCGTCTTAAGGATCGTGTTCAGCCTGAGATTCTT |
| | | GAGGCTCTTCTTAAGCATATTTCTTTTGATAAGTTTGTTCAGATTTCT |
| | | CTTAAGGCTCTTCGTCGTATTGTTCCTCTTATGGAGCAGGGTAAGCG |
| | | TTATGATGAGGCTTGTGCTGAGATTTATGGTGATCATTATGGTAAGA |
| | | AGAATACTGAGGAGAAGATTTATCTTCCTCCTATTCCTGCTGATGAG |
| | | ATTCGTAATCCTGTTGTTCTTCGTGCTCTTTCTCAGGCTCGTAAGGTT |
| | | ATTAATGGTGTTGTTCGTCGTTATGGTTCTCCTGCTCGTATTCATATT |
| | | GAGACTGCTCGTGAGGTTGGTAAGTCTTTTAAGGATCGTAAGGAGA |
| | | TTGAGAAGCGTCAGGAGGAGAATCGTAAGGATCGTGAGAAGGCTG |
| | | CTGCTAAGTTTCGTGAGTATTTTCCTAATTTTGTTGGTGAGCCTAAG |
| | | TCTAAGGATATTCTTAAGCTTCGTCTTTATGAGCAGCAGCATGGTAA |
| | | GTGTCTTTATTCTGGTAAGGAGATTAATCTTGTTCGTCTTAATGAGA |
| | | AGGGTTATGTTGAGATTGATCATGCTCTTCCTTTTTCTCGTACTTGG |
| | | GATGATTCTTTTAATAATAAGGTTCTTGTTCTTGGTTCTGAGAATCA |
| | | GAATAAGGGTAATCAGACTCCTTATGAGTATTTTAATGGTAAGGAT |
| | | AATTCTCGTGAGTGGCAGGAGTTTAAGGCTCGTGTTGAGACTTCTC |
| | | GTTTTCCTCGTTCTAAGAAGCAGCGTATTCTTCTTCAGAAGTTTGAT |
| | | GAGGATGGTTTTAAGGAGTGTAATCTTAATGATACTCGTTATGTTAA |
| | | TCGTTTTCTTTGTCAGTTTGTTGCTGATCATATTCTTCTTACTGGTAA |
| | | GGGTAAGCGTCGTGTTTTTGCTTCTAATGGTCAGATTACTAATCTTC |
| | | TTCGTGGTTTTTGGGGTCTTCGTAAGGTTCGTGCTGAGAATGATCGT |
| | | CATCATGCTCTTGATGCTGTTGTTGTTGCTTGTTCTACTGTTGCTATG |
| | | CAGCAGAAGATTACTCGTTTTGTTCGTTATAAGGAGATGAATGCTTT |
| | | TGATGGTAAGACTATTGATAAGGAGACTGGTAAGGTTCTTCATCAG |
| | | AAGACTCATTTTCCTCAGCCTTGGGAGTTTTTTGCTCAGGAGGTTAT |
| | | GATTCGTGTTTTTGGTAAGCCTGATGGTAAGCCTGAGTTTGAGGAG |
| | | GCTGATACTCCTGAGAAGCTTCGTACTCTTCTTGCTGAGAAGCTTTC |
| | | TTCTCGTCCTGAGGCTGTTCATGAGTATGTTACTCCTCTTTTTGTTTC |
| | | TCGTGCTCCTAATCGTAAGATGTCTGGTGCTCATAAGGATACTCTTC |
| | | GTTCTGCTAAGCGTTTTGTTAAGCATAATGAGAAGATTTCTGTTAAG |
| | | CGTGTTTGGCTTACTGAGATTAAGCTTGCTGATCTTGAGAATATGGT |
| | | TAATTATAAGAATGGTCGTGAGATTGAGCTTTATGAGGCTCTTAAG |
| | | GCTCGTCTTGAGGCTTATGGTGGTAATGCTAAGCAGGCTTTTGATCC |
| | | TAAGGATAATCCTTTTTATAAGAAGGGTGGTCAGCTTGTTAAGGCT |
| | | GTTCGTGTTGAGAAGACTCAGGAGTCTGGTGTTCTTCTTAATAAGA |
| | | AGAATGCTTATACTATTGCTGATAATGGTGATATGGTTCGTGTTGAT |
| | | GTTTTTTGTAAGGTTGATAAGAAGGGTAAGAATCAGTATTTTATTGT |
| | | TCCTATTTATGCTTGGCAGGTTGCTGAGAATATTCTTCCTGATATTG |
| | | ATTGTAAGGGTTATCGTATTGATGATTCTTATACTTTTTGTTTTTCTC |
| | | TTCATAAGTATGATCTTATTGCTTTTCAGAAGGATGAGAAGTCTAAG |
| | | GTTGAGTTTGCTTATTATATTAATTGTGATTCTTCTAATGGTCGTTTT |
| | | TATCTTGCTTGGCATGATAAGGGTTCTAAGGAGCAGCAGTTTCGTAT |
| | | TTCTACTCAGAATCTTGTTCTTATTCAGAAGTATCAGGTTAATGAGC |
| | | TTGGTAAGGAGATTCGTCCTTGTCGTCTTAAGAAGCGTCCTCCTGTT |
| | | CGTUGA |
| 210 | Exemplary open | ATGGCCGCCTTCAAGCCCAACCCCATCAACTACATCCTGGGCCTGG |
| | reading frame for | ACATCGGCATCGCCTCCGTGGGCTGGGCCATGGTGGAGATCGACGA |
| | Nme2Cas9 | GGAGGAGAACCCCATCCGGCTGATCGACCTGGGCGTGCGGGTGTTC |
| | cleavase | GAGCGGGCCGAGGTGCCCAAGACCGGCGACTCCCTGGCCATGGCCC |
| | | GGCGGCTGGCCCGGTCCGTGCGGCGGCTGACCCGGCGGCGGGCCCA |
| | | CCGGCTGCTGCGGGCCCGGCGGCTGCTGAAGCGGGAGGGCGTGCTG |
| | | CAGGCCGCCGACTTCGACGAGAACGGCCTGATCAAGTCCCTGCCCA |
| | | ACACCCCCTGGCAGCTGCGGGCCGCCGCCCTGGACCGGAAGCTGAC |
| | | CCCCCTGGAGTGGTCCGCCGTGCTGCTGCACCTGATCAAGCACCGG |
| | | GGCTACCTGTCCCAGCGGAAGAACGAGGGCGAGACCGCCGACAAG |
| | | GAGCTGGGCGCCCTGCTGAAGGGCGTGGCCAACAACGCCCACGCCC |
| | | TGCAGACCGGCGACTTCCGGACCCCCGCCGAGCTGGCCCTGAACAA |
| | | GTTCGAGAAGGAGTCCGGCCACATCCGGAACCAGCGGGGCGACTA |
| | | CTCCCACACCTTCTCCCGGAAGGACCTGCAGGCCGAGCTGATCCTG |
| | | CTGTTCGAGAAGCAGAAGGAGTTCGGCAACCCCCACGTGTCCGGCG |
| | | GCCTGAAGGAGGGCATCGAGACCCTGCTGATGACCCAGCGGCCCGC |
| | | CCTGTCCGGCGACGCCGTGCAGAAGATGCTGGGCCACTGCACCTTC |
| | | GAGCCCGCCGAGCCCAAGGCCGCCAAGAACACCTACACCGCCGAG |
| | | CGGTTCATCTGGCTGACCAAGCTGAACAACCTGCGGATCCTGGAGC |
| | | AGGGCTCCGAGCGGCCCCTGACCGACACCGAGCGGGCCACCCTGAT |
| | | GGACGAGCCCTACCGGAAGTCCAAGCTGACCTACGCCCAGGCCCGG |
| | | AAGCTGCTGGGCCTGGAGGACACCGCCTTCTTCAAGGGCCTGCGGT |
| | | ACGGCAAGGACAACGCCGAGGCCTCCACCCTGATGGAGATGAAGG |
| | | CCTACCACGCCATCTCCCGGGCCCTGGAGAAGGAGGGCCTGAAGGA |
| | | CAAGAAGTCCCCCCTGAACCTGTCCTCCGAGCTGCAGGACGAGATC |
| | | GGCACCGCCTTCTCCCTGTTCAAGACCGACGAGGACATCACCGGCC |
| | | GGCTGAAGGACCGGGTGCAGCCCGAGATCCTGGAGGCCCTGCTGA |
| | | AGCACATCTCCTTCGACAAGTTCGTGCAGATCTCCCTGAAGGCCCT |
| | | GCGGCGGATCGTGCCCCTGATGGAGCAGGGCAAGCGGTACGACGA |
| | | GGCCTGCGCCGAGATCTACGGCGACCACTACGGCAAGAAGAACAC |
| | | CGAGGAGAAGATCTACCTGCCCCCCATCCCCGCCGACGAGATCCGG |
| | | AACCCCGTGGTGCTGCGGGCCCTGTCCCAGGCCCGGAAGGTGATCA |
| | | ACGGCGTGGTGCGGCGGTACGGCTCCCCCGCCCGGATCCACATCGA |
| | | GACCGCCCGGGAGGTGGGCAAGTCCTTCAAGGACCGGAAGGAGAT |
| | | CGAGAAGCGGCAGGAGGAGAACCGGAAGGACCGGGAGAAGGCCG |
| | | CCGCCAAGTTCCGGGAGTACTTCCCCAACTTCGTGGGCGAGCCCAA |
| | | GTCCAAGGACATCCTGAAGCTGCGGCTGTACGAGCAGCAGCACGGC |
| | | AAGTGCCTGTACTCCGGCAAGGAGATCAACCTGGTGCGGCTGAACG |
| | | AGAAGGGCTACGTGGAGATCGACCACGCCCTGCCCTTCTCCCGGAC |
| | | CTGGGACGACTCCTTCAACAACAAGGTGCTGGTGCTGGGCTCCGAG |
| | | AACCAGAACAAGGGCAACCAGACCCCCTACGAGTACTTCAACGGC |
| | | AAGGACAACTCCCGGGAGTGGCAGGAGTTCAAGGCCCGGGTGGAG |
| | | ACCTCCCGGTTCCCCCGGTCCAAGAAGCAGCGGATCCTGCTGCAGA |
| | | AGTTCGACGAGGACGGCTTCAAGGAGTGCAACCTGAACGACACCC |
| | | GGTACGTGAACCGGTTCCTGTGCCAGTTCGTGGCCGACCACATCCT |
| | | GCTGACCGGCAAGGGCAAGCGGCGGGTGTTCGCCTCCAACGGCCA |
| | | GATCACCAACCTGCTGCGGGGCTTCTGGGGCCTGCGGAAGGTGCGG |
| | | GCCGAGAACGACCGGCACCACGCCCTGGACGCCGTGGTGGTGGCCT |
| | | GCTCCACCGTGGCCATGCAGCAGAAGATCACCCGGTTCGTGCGGTA |
| | | CAAGGAGATGAACGCCTTCGACGGCAAGACCATCGACAAGGAGAC |
| | | CGGCAAGGTGCTGCACCAGAAGACCCACTTCCCCCAGCCCTGGGAG |
| | | TTCTTCGCCCAGGAGGTGATGATCCGGGTGTTCGGCAAGCCCGACG |
| | | GCAAGCCCGAGTTCGAGGAGGCCGACACCCCCGAGAAGCTGCGGA |
| | | CCCTGCTGGCCGAGAAGCTGTCCTCCCGGCCCGAGGCCGTGCACGA |
| | | GTACGTGACCCCCCTGTTCGTGTCCCGGGCCCCCAACCGGAAGATG |
| | | TCCGGCGCCCACAAGGACACCCTGCGGTCCGCCAAGCGGTTCGTGA |
| | | AGCACAACGAGAAGATCTCCGTGAAGCGGGTGTGGCTGACCGAGA |
| | | TCAAGCTGGCCGACCTGGAGAACATGGTGAACTACAAGAACGGCC |
| | | GGGAGATCGAGCTGTACGAGGCCCTGAAGGCCCGGCTGGAGGCCT |
| | | ACGGCGGCAACGCCAAGCAGGCCTTCGACCCCAAGGACAACCCCTT |
| | | CTACAAGAAGGGCGGCCAGCTGGTGAAGGCCGTGCGGGTGGAGAA |
| | | GACCCAGGAGTCCGGCGTGCTGCTGAACAAGAAGAACGCCTACAC |
| | | CATCGCCGACAACGGCGACATGGTGCGGGTGGACGTGTTCTGCAAG |
| | | GTGGACAAGAAGGGCAAGAACCAGTACTTCATCGTGCCCATCTACG |
| | | CCTGGCAGGTGGCCGAGAACATCCTGCCCGACATCGACTGCAAGGG |
| | | CTACCGGATCGACGACTCCTACACCTTCTGCTTCTCCCTGCACAAGT |
| | | ACGACCTGATCGCCTTCCAGAAGGACGAGAAGTCCAAGGTGGAGTT |
| | | CGCCTACTACATCAACTGCGACTCCTCCAACGGCCGGTTCTACCTGG |
| | | CCTGGCACGACAAGGGCTCCAAGGAGCAGCAGTTCCGGATCTCCAC |
| | | CCAGAACCTGGTGCTGATCCAGAAGTACCAGGTGAACGAGCTGGGC |
| | | AAGGAGATCCGGCCCTGCCGGCTGAAGAAGCGGCCCCCCGTGCGG |
| | | UGA |
| 211 | Exemplary open | ATGGCAGCATTCAAACCAAACCCAATCAACTACATCCTAGGACTAG |
| | reading frame for | ACATCGGAATCGCATCAGTAGGATGAGCAATGGTAGAAATCGACG |
| | Nme2Cas9 | AAGAAGAAAACCCAATCCGACTAATCGACCTAGGAGTACGAGTATT |
| | cleavase | CGAACGAGCAGAAGTACCAAAAACAGGAGACTCACTAGCAATGGC |
| | | ACGACGACTAGCACGATCAGTACGACGACTAACACGACGACGAGC |
| | | ACACCGACTACTACGAGCACGACGACTACTAAAACGAGAAGGAGT |
| | | ACTACAAGCAGCAGACTTCGACGAAAACGGACTAATCAAATCACTA |
| | | CCAAACACACCATGACAACTACGAGCAGCAGCACTAGACCGAAAA |
| | | CTAACACCACTAGAATGATCAGCAGTACTACTACACCTAATCAAAC |
| | | ACCGAGGATACCTATCACAACGAAAAAACGAAGGAGAAACAGCAG |
| | | ACAAAGAACTAGGAGCACTACTAAAAGGAGTAGCAAACAACGCAC |
| | | ACGCACTACAAACAGGAGACTTCCGAACACCAGCAGAACTAGCAC |
| | | TAAACAAATTCGAAAAAGAATCAGGACACATCCGAAACCAACGAG |
| | | GAGACTACTCACACACATTCTCACGAAAAGACCTACAAGCAGAACT |
| | | AATCCTACTATTCGAAAAACAAAAAGAATTCGGAAACCCACACGTA |
| | | TCAGGAGGACTAAAAGAAGGAATCGAAACACTACTAATGACACAA |
| | | CGACCAGCACTATCAGGAGACGCAGTACAAAAAATGCTAGGACAC |
| | | TGCACATTCGAACCAGCAGAACCAAAAGCAGCAAAAAACACATAC |
| | | ACAGCAGAACGATTCATCTGACTAACAAAACTAAACAACCTACGAA |
| | | TCCTAGAACAAGGATCAGAACGACCACTAACAGACACAGAACGAG |
| | | CAACACTAATGGACGAACCATACCGAAAATCAAAACTAACATACG |
| | | CACAAGCACGAAAACTACTAGGACTAGAAGACACAGCATTCTTCAA |
| | | AGGACTACGATACGGAAAAGACAACGCAGAAGCATCAACACTAAT |
| | | GGAAATGAAAGCATACCACGCAATCTCACGAGCACTAGAAAAAGA |
| | | AGGACTAAAAGACAAAAAATCACCACTAAACCTATCATCAGAACT |
| | | ACAAGACGAAATCGGAACAGCATTCTCACTATTCAAAACAGACGA |
| | | AGACATCACAGGACGACTAAAAGACCGAGTACAACCAGAAATCCT |
| | | AGAAGCACTACTAAAACACATCTCATTCGACAAATTCGTACAAATC |
| | | TCACTAAAAGCACTACGACGAATCGTACCACTAATGGAACAAGGA |
| | | AAACGATACGACGAAGCATGCGCAGAAATCTACGGAGACCACTAC |
| | | GGAAAAAAAAACACAGAAGAAAAAATCTACCTACCACCAATCCCA |
| | | GCAGACGAAATCCGAAACCCAGTAGTACTACGAGCACTATCACAA |
| | | GCACGAAAAGTAATCAACGGAGTAGTACGACGATACGGATCACCA |
| | | GCACGAATCCACATCGAAACAGCACGAGAAGTAGGAAAATCATTC |
| | | AAAGACCGAAAAGAAATCGAAAAACGACAAGAAGAAAACCGAAA |
| | | AGACCGAGAAAAAGCAGCAGCAAAATTCCGAGAATACTTCCCAAA |
| | | CTTCGTAGGAGAACCAAAATCAAAAGACATCCTAAAACTACGACTA |
| | | TACGAACAACAACACGGAAAATGCCTATACTCAGGAAAAGAAATC |
| | | AACCTAGTACGACTAAACGAAAAAGGATACGTAGAAATCGACCAC |
| | | GCACTACCATTCTCACGAACATGAGACGACTCATTCAACAACAAAG |
| | | TACTAGTACTAGGATCAGAAAACCAAAACAAAGGAAACCAAACAC |
| | | CATACGAATACTTCAACGGAAAAGACAACTCACGAGAATGACAAG |
| | | AATTCAAAGCACGAGTAGAAACATCACGATTCCCACGATCAAAAA |
| | | AACAACGAATCCTACTACAAAAATTCGACGAAGACGGATTCAAAG |
| | | AATGCAACCTAAACGACACACGATACGTAAACCGATTCCTATGCCA |
| | | ATTCGTAGCAGACCACATCCTACTAACAGGAAAAGGAAAACGACG |
| | | AGTATTCGCATCAAACGGACAAATCACAAACCTACTACGAGGATTC |
| | | TGAGGACTACGAAAAGTACGAGCAGAAAACGACCGACACCACGCA |
| | | CTAGACGCAGTAGTAGTAGCATGCTCAACAGTAGCAATGCAACAAA |
| | | AAATCACACGATTCGTACGATACAAAGAAATGAACGCATTCGACGG |
| | | AAAAACAATCGACAAAGAAACAGGAAAAGTACTACACCAAAAAAC |
| | | ACACTTCCCACAACCATGAGAATTCTTCGCACAAGAAGTAATGATC |
| | | CGAGTATTCGGAAAACCAGACGGAAAACCAGAATTCGAAGAAGCA |
| | | GACACACCAGAAAAACTACGAACACTACTAGCAGAAAAACTATCA |
| | | TCACGACCAGAAGCAGTACACGAATACGTAACACCACTATTCGTAT |
| | | CACGAGCACCAAACCGAAAAATGTCAGGAGCACACAAAGACACAC |
| | | TACGATCAGCAAAACGATTCGTAAAACACAACGAAAAAATCTCAGT |
| | | AAAACGAGTATGACTAACAGAAATCAAACTAGCAGACCTAGAAAA |
| | | CATGGTAAACTACAAAAACGGACGAGAAATCGAACTATACGAAGC |
| | | ACTAAAAGCACGACTAGAAGCATACGGAGGAAACGCAAAACAAGC |
| | | ATTCGACCCAAAAGACAACCCATTCTACAAAAAAGGAGGACAACT |
| | | AGTAAAAGCAGTACGAGTAGAAAAAACACAAGAATCAGGAGTACT |
| | | ACTAAACAAAAAAAACGCATACACAATCGCAGACAACGGAGACAT |
| | | GGTACGAGTAGACGTATTCTGCAAAGTAGACAAAAAAGGAAAAAA |
| | | CCAATACTTCATCGTACCAATCTACGCATGACAAGTAGCAGAAAAC |
| | | ATCCTACCAGACATCGACTGCAAAGGATACCGAATCGACGACTCAT |
| | | ACACATTCTGCTTCTCACTACACAAATACGACCTAATCGCATTCCAA |
| | | AAAGACGAAAAATCAAAAGTAGAATTCGCATACTACATCAACTGC |
| | | GACTCATCAAACGGACGATTCTACCTAGCATGACACGACAAAGGAT |
| | | CAAAAGAACAACAATTCCGAATCTCAACACAAAACCTAGTACTAAT |
| | | CCAAAAATACCAAGTAAACGAACTAGGAAAAGAAATCCGACCATG |
| | | CCGACTAAAAAAACGACCACCAGTACGAUAA |
| 212 | Exemplary amino | MAAFKPNPINYILGLDIGIASVGWAMVEIDEEENPIRLIDLGVRVFERAE |
| | acid sequence of | VPKTGDSLAMARRLARSVRRLTRRRAHRLLRARRLLKREGVLQAADF |
| | Nme3Cas9 | DENGLIKSLPNTPWQLRAAALDRKLTPLEWSAVLLHLIKHRGYLSQRK |
| | cleavase | NEGETADKELGALLKGVADNAHALQTGDFRTPAELALNKFEKECGHI |
| | | RNQRGDYSHTFSRKDLQAELNLLFEKQKEFGNPHVSGGLKEGIETLLM |
| | | TQRPALSGDAVQKMLGHCTFEPAEPKAAKNTYTAERFIWLTKLNNLRI |
| | | LEQGSERPLTDTERATLMDEPYRKSKLTYAQARKLLSLEDTAFFKGLR |
| | | YGKDNAEASTLMEMKAYHTISRALEKEGLKDKKSPLNLSPELQDEIGT |
| | | AFSLFKTDEDITGRLKDRIQPEILEALLKHISFDKFVQISLKALRRIVPLM |
| | | EQGKRYDEACAEIYGDHYGKKNTEEKIYLPPIPADEIRNPVVLRALSQA |
| | | RKVINGVVRRYGSPARIHIETAREVGKSFKDRKEIEKRQEENRKDREKA |
| | | AAKFREYFPNFVGEPKSKDILKLRLYEQQHGKCLYSGKEINLGRLNEK |
| | | GYVEIDHALPFSRTWDDSFNNKVLVLGSENQNKGNQTPYEYFNGKDN |
| | | SREWQEFKARVETSRFPRSKKQRILLQKFDEDGFKERNLNDTRYVNRF |
| | | LCQFVADRMRLTGKGKKRVFASNGQITNLLRGFWGLRKVRAENDRH |
| | | HALDAVVVACSTVAMQQKITRFVRYKEMNAFDGKTIDKETGEVLHQ |
| | | KTHFPQPWEFFAQEVMIRVFGKPDGKPEFEEADTPEKLRTLLAEKLSSR |
| | | PEAVHEYVTPLFVSRAPNRKMSGQGHMETVKSAKRLDEGVSVLRVPL |
| | | TQLKLKDLEKMVNREREPKLYEALKARLEAHKDDPAKAFAEPFYKYD |
| | | KAGNRTQQVKAVRVEQVQKTGVWVRNHNGIADNATMVRVDVFEKG |
| | | DKYYLVPIYSWQVAKGILPDRAVVAYADEEDWTVIDESFRFKFVLYSN |
| | | DLIKVQLKKDSFLGYFSGLDRATGAISLREHDLEKSKGKDGMHRIGVK |
| | | TALSFQKYQIDEMGKEIRPCRLKKRPPVR |
| 213 | Exemplary | GCTGCTTTTAAGCCTAATCCTATTAATTATATTCTTGGTCTTGATATT |
| | coding sequence | GGTATTGCTTCTGTTGGTTGGGCTATGGTTGAGATTGATGAGGAGG |
| | encoding | AGAATCCTATTCGTCTTATTGATCTTGGTGTTCGTGTTTTTGAGCGT |
| | Nme3Cas9 | GCTGAGGTTCCTAAGACTGGTGATTCTCTTGCTATGGCTCGTCGTCT |
| | cleavase | TGCTCGTTCTGTTCGTCGTCTTACTCGTCGTCGTGCTCATCGTCTTCT |
| | | TCGTGCTCGTCGTCTTCTTAAGCGTGAGGGTGTTCTTCAGGCTGCTG |
| | | ATTTTGATGAGAATGGTCTTATTAAGTCTCTTCCTAATACTCCTTGG |
| | | CAGCTTCGTGCTGCTGCTCTTGATCGTAAGCTTACTCCTCTTGAGTG |
| | | GTCTGCTGTTCTTCTTCATCTTATTAAGCATCGTGGTTATCTTTCTCA |
| | | GCGTAAGAATGAGGGTGAGACTGCTGATAAGGAGCTTGGTGCTCTT |
| | | CTTAAGGGTGTTGCTGATAATGCTCATGCTCTTCAGACTGGTGATTT |
| | | TCGTACTCCTGCTGAGCTTGCTCTTAATAAGTTTGAGAAGGAGTGTG |
| | | GTCATATTCGTAATCAGCGTGGTGATTATTCTCATACTTTTTCTCGT |
| | | AAGGATCTTCAGGCTGAGCTTAATCTTCTTTTTGAGAAGCAGAAGG |
| | | AGTTTGGTAATCCTCATGTTTCTGGTGGTCTTAAGGAGGGTATTGAG |
| | | ACTCTTCTTATGACTCAGCGTCCTGCTCTTTCTGGTGATGCTGTTCA |
| | | GAAGATGCTTGGTCATTGTACTTTTGAGCCTGCTGAGCCTAAGGCTG |
| | | CTAAGAATACTTATACTGCTGAGCGTTTTATTTGGCTTACTAAGCTT |
| | | AATAATCTTCGTATTCTTGAGCAGGGTTCTGAGCGTCCTCTTACTGA |
| | | TACTGAGCGTGCTACTCTTATGGATGAGCCTTATCGTAAGTCTAAGC |
| | | TTACTTATGCTCAGGCTCGTAAGCTTCTTTCTCTTGAGGATACTGCT |
| | | TTTTTTAAGGGTCTTCGTTATGGTAAGGATAATGCTGAGGCTTCTAC |
| | | TCTTATGGAGATGAAGGCTTATCATACTATTTCTCGTGCTCTTGAGA |
| | | AGGAGGGTCTTAAGGATAAGAAGTCTCCTCTTAATCTTTCTCCTGAG |
| | | CTTCAGGATGAGATTGGTACTGCTTTTTCTCTTTTTAAGACTGATGA |
| | | GGATATTACTGGTCGTCTTAAGGATCGTATTCAGCCTGAGATTCTTG |
| | | AGGCTCTTCTTAAGCATATTTCTTTTGATAAGTTTGTTCAGATTTCTC |
| | | TTAAGGCTCTTCGTCGTATTGTTCCTCTTATGGAGCAGGGTAAGCGT |
| | | TATGATGAGGCTTGTGCTGAGATTTATGGTGATCATTATGGTAAGA |
| | | AGAATACTGAGGAGAAGATTTATCTTCCTCCTATTCCTGCTGATGAG |
| | | ATTCGTAATCCTGTTGTTCTTCGTGCTCTTTCTCAGGCTCGTAAGGTT |
| | | ATTAATGGTGTTGTTCGTCGTTATGGTTCTCCTGCTCGTATTCATATT |
| | | GAGACTGCTCGTGAGGTTGGTAAGTCTTTTAAGGATCGTAAGGAGA |
| | | TTGAGAAGCGTCAGGAGGAGAATCGTAAGGATCGTGAGAAGGCTG |
| | | CTGCTAAGTTTCGTGAGTATTTTCCTAATTTTGTTGGTGAGCCTAAG |
| | | TCTAAGGATATTCTTAAGCTTCGTCTTTATGAGCAGCAGCATGGTAA |
| | | GTGTCTTTATTCTGGTAAGGAGATTAATCTTGGTCGTCTTAATGAGA |
| | | AGGGTTATGTTGAGATTGATCATGCTCTTCCTTTTTCTCGTACTTGG |
| | | GATGATTCTTTTAATAATAAGGTTCTTGTTCTTGGTTCTGAGAATCA |
| | | GAATAAGGGTAATCAGACTCCTTATGAGTATTTTAATGGTAAGGAT |
| | | AATTCTCGTGAGTGGCAGGAGTTTAAGGCTCGTGTTGAGACTTCTC |
| | | GTTTTCCTCGTTCTAAGAAGCAGCGTATTCTTCTTCAGAAGTTTGAT |
| | | GAGGATGGTTTTAAGGAGCGTAATCTTAATGATACTCGTTATGTTA |
| | | ATCGTTTTCTTTGTCAGTTTGTTGCTGATCGTATGCGTCTTACTGGTA |
| | | AGGGTAAGAAGCGTGTTTTTGCTTCTAATGGTCAGATTACTAATCTT |
| | | CTTCGTGGTTTTTGGGGTCTTCGTAAGGTTCGTGCTGAGAATGATCG |
| | | TCATCATGCTCTTGATGCTGTTGTTGTTGCTTGTTCTACTGTTGCTAT |
| | | GCAGCAGAAGATTACTCGTTTTGTTCGTTATAAGGAGATGAATGCT |
| | | TTTGATGGTAAGACTATTGATAAGGAGACTGGTGAGGTTCTTCATC |
| | | AGAAGACTCATTTTCCTCAGCCTTGGGAGTTTTTTGCTCAGGAGGTT |
| | | ATGATTCGTGTTTTTGGTAAGCCTGATGGTAAGCCTGAGTTTGAGGA |
| | | GGCTGATACTCCTGAGAAGCTTCGTACTCTTCTTGCTGAGAAGCTTT |
| | | CTTCTCGTCCTGAGGCTGTTCATGAGTATGTTACTCCTCTTTTTGTTT |
| | | CTCGTGCTCCTAATCGTAAGATGTCTGGTCAGGGTCATATGGAGAC |
| | | TGTTAAGTCTGCTAAGCGTCTTGATGAGGGTGTTTCTGTTCTTCGTG |
| | | TTCCTCTTACTCAGCTTAAGCTTAAGGATCTTGAGAAGATGGTTAAT |
| | | CGTGAGCGTGAGCCTAAGCTTTATGAGGCTCTTAAGGCTCGTCTTG |
| | | AGGCTCATAAGGATGATCCTGCTAAGGCTTTTGCTGAGCCTTTTTAT |
| | | AAGTATGATAAGGCTGGTAATCGTACTCAGCAGGTTAAGGCTGTTC |
| | | GTGTTGAGCAGGTTCAGAAGACTGGTGTTTGGGTTCGTAATCATAA |
| | | TGGTATTGCTGATAATGCTACTATGGTTCGTGTTGATGTTTTTGAGA |
| | | AGGGTGATAAGTATTATCTTGTTCCTATTTATTCTTGGCAGGTTGCT |
| | | AAGGGTATTCTTCCTGATCGTGCTGTTGTTGCTTATGCTGATGAGGA |
| | | GGATTGGACTGTTATTGATGAGTCTTTTCGTTTTAAGTTTGTTCTTTA |
| | | TTCTAATGATCTTATTAAGGTTCAGCTTAAGAAGGATTCTTTTCTTG |
| | | GTTATTTTTCTGGTCTTGATCGTGCTACTGGTGCTATTTCTCTTCGTG |
| | | AGCATGATCTTGAGAAGTCTAAGGGTAAGGATGGTATGCATCGTAT |
| | | TGGTGTTAAGACTGCTCTTTCTTTTCAGAAGTATCAGATTGATGAGA |
| | | TGGGTAAGGAGATTCGTCCTTGTCGTCTTAAGAAGCGTCCTCCTGTT |
| | | CGT |
| 214 | Exemplary | GCCGCCTTCAAGCCCAACCCCATCAACTACATCCTGGGCCTGGACA |
| | coding sequence | TCGGCATCGCCTCCGTGGGCTGGGCCATGGTGGAGATCGACGAGGA |
| | encoding | GGAGAACCCCATCCGGCTGATCGACCTGGGCGTGCGGGTGTTCGAG |
| | Nme3Cas9 | CGGGCCGAGGTGCCCAAGACCGGCGACTCCCTGGCCATGGCCCGGC |
| | cleavase | GGCTGGCCCGGTCCGTGCGGCGGCTGACCCGGCGGCGGGCCCACCG |
| | | GCTGCTGCGGGCCCGGCGGCTGCTGAAGCGGGAGGGCGTGCTGCA |
| | | GGCCGCCGACTTCGACGAGAACGGCCTGATCAAGTCCCTGCCCAAC |
| | | ACCCCCTGGCAGCTGCGGGCCGCCGCCCTGGACCGGAAGCTGACCC |
| | | CCCTGGAGTGGTCCGCCGTGCTGCTGCACCTGATCAAGCACCGGGG |
| | | CTACCTGTCCCAGCGGAAGAACGAGGGCGAGACCGCCGACAAGGA |
| | | GCTGGGCGCCCTGCTGAAGGGCGTGGCCGACAACGCCCACGCCCTG |
| | | CAGACCGGCGACTTCCGGACCCCCGCCGAGCTGGCCCTGAACAAGT |
| | | TCGAGAAGGAGTGCGGCCACATCCGGAACCAGCGGGGCGACTACT |
| | | CCCACACCTTCTCCCGGAAGGACCTGCAGGCCGAGCTGAACCTGCT |
| | | GTTCGAGAAGCAGAAGGAGTTCGGCAACCCCCACGTGTCCGGCGGC |
| | | CTGAAGGAGGGCATCGAGACCCTGCTGATGACCCAGCGGCCCGCCC |
| | | TGTCCGGCGACGCCGTGCAGAAGATGCTGGGCCACTGCACCTTCGA |
| | | GCCCGCCGAGCCCAAGGCCGCCAAGAACACCTACACCGCCGAGCG |
| | | GTTCATCTGGCTGACCAAGCTGAACAACCTGCGGATCCTGGAGCAG |
| | | GGCTCCGAGCGGCCCCTGACCGACACCGAGCGGGCCACCCTGATGG |
| | | ACGAGCCCTACCGGAAGTCCAAGCTGACCTACGCCCAGGCCCGGAA |
| | | GCTGCTGTCCCTGGAGGACACCGCCTTCTTCAAGGGCCTGCGGTAC |
| | | GGCAAGGACAACGCCGAGGCCTCCACCCTGATGGAGATGAAGGCC |
| | | TACCACACCATCTCCCGGGCCCTGGAGAAGGAGGGCCTGAAGGAC |
| | | AAGAAGTCCCCCCTGAACCTGTCCCCCGAGCTGCAGGACGAGATCG |
| | | GCACCGCCTTCTCCCTGTTCAAGACCGACGAGGACATCACCGGCCG |
| | | GCTGAAGGACCGGATCCAGCCCGAGATCCTGGAGGCCCTGCTGAAG |
| | | CACATCTCCTTCGACAAGTTCGTGCAGATCTCCCTGAAGGCCCTGCG |
| | | GCGGATCGTGCCCCTGATGGAGCAGGGCAAGCGGTACGACGAGGC |
| | | CTGCGCCGAGATCTACGGCGACCACTACGGCAAGAAGAACACCGA |
| | | GGAGAAGATCTACCTGCCCCCCATCCCCGCCGACGAGATCCGGAAC |
| | | CCCGTGGTGCTGCGGGCCCTGTCCCAGGCCCGGAAGGTGATCAACG |
| | | GCGTGGTGCGGCGGTACGGCTCCCCCGCCCGGATCCACATCGAGAC |
| | | CGCCCGGGAGGTGGGCAAGTCCTTCAAGGACCGGAAGGAGATCGA |
| | | GAAGCGGCAGGAGGAGAACCGGAAGGACCGGGAGAAGGCCGCCG |
| | | CCAAGTTCCGGGAGTACTTCCCCAACTTCGTGGGCGAGCCCAAGTC |
| | | CAAGGACATCCTGAAGCTGCGGCTGTACGAGCAGCAGCACGGCAA |
| | | GTGCCTGTACTCCGGCAAGGAGATCAACCTGGGCCGGCTGAACGAG |
| | | AAGGGCTACGTGGAGATCGACCACGCCCTGCCCTTCTCCCGGACCT |
| | | GGGACGACTCCTTCAACAACAAGGTGCTGGTGCTGGGCTCCGAGAA |
| | | CCAGAACAAGGGCAACCAGACCCCCTACGAGTACTTCAACGGCAA |
| | | GGACAACTCCCGGGAGTGGCAGGAGTTCAAGGCCCGGGTGGAGAC |
| | | CTCCCGGTTCCCCCGGTCCAAGAAGCAGCGGATCCTGCTGCAGAAG |
| | | TTCGACGAGGACGGCTTCAAGGAGCGGAACCTGAACGACACCCGG |
| | | TACGTGAACCGGTTCCTGTGCCAGTTCGTGGCCGACCGGATGCGGC |
| | | TGACCGGCAAGGGCAAGAAGCGGGTGTTCGCCTCCAACGGCCAGA |
| | | TCACCAACCTGCTGCGGGGCTTCTGGGGCCTGCGGAAGGTGCGGGC |
| | | CGAGAACGACCGGCACCACGCCCTGGACGCCGTGGTGGTGGCCTGC |
| | | TCCACCGTGGCCATGCAGCAGAAGATCACCCGGTTCGTGCGGTACA |
| | | AGGAGATGAACGCCTTCGACGGCAAGACCATCGACAAGGAGACCG |
| | | GCGAGGTGCTGCACCAGAAGACCCACTTCCCCCAGCCCTGGGAGTT |
| | | CTTCGCCCAGGAGGTGATGATCCGGGTGTTCGGCAAGCCCGACGGC |
| | | AAGCCCGAGTTCGAGGAGGCCGACACCCCCGAGAAGCTGCGGACC |
| | | CTGCTGGCCGAGAAGCTGTCCTCCCGGCCCGAGGCCGTGCACGAGT |
| | | ACGTGACCCCCCTGTTCGTGTCCCGGGCCCCCAACCGGAAGATGTC |
| | | CGGCCAGGGCCACATGGAGACCGTGAAGTCCGCCAAGCGGCTGGA |
| | | CGAGGGCGTGTCCGTGCTGCGGGTGCCCCTGACCCAGCTGAAGCTG |
| | | AAGGACCTGGAGAAGATGGTGAACCGGGAGCGGGAGCCCAAGCTG |
| | | TACGAGGCCCTGAAGGCCCGGCTGGAGGCCCACAAGGACGACCCC |
| | | GCCAAGGCCTTCGCCGAGCCCTTCTACAAGTACGACAAGGCCGGCA |
| | | ACCGGACCCAGCAGGTGAAGGCCGTGCGGGTGGAGCAGGTGCAGA |
| | | AGACCGGCGTGTGGGTGCGGAACCACAACGGCATCGCCGACAACG |
| | | CCACCATGGTGCGGGTGGACGTGTTCGAGAAGGGCGACAAGTACTA |
| | | CCTGGTGCCCATCTACTCCTGGCAGGTGGCCAAGGGCATCCTGCCC |
| | | GACCGGGCCGTGGTGGCCTACGCCGACGAGGAGGACTGGACCGTG |
| | | ATCGACGAGTCCTTCCGGTTCAAGTTCGTGCTGTACTCCAACGACCT |
| | | GATCAAGGTGCAGCTGAAGAAGGACTCCTTCCTGGGCTACTTCTCC |
| | | GGCCTGGACCGGGCCACCGGCGCCATCTCCCTGCGGGAGCACGACC |
| | | TGGAGAAGTCCAAGGGCAAGGACGGCATGCACCGGATCGGCGTGA |
| | | AGACCGCCCTGTCCTTCCAGAAGTACCAGATCGACGAGATGGGCAA |
| | | GGAGATCCGGCCCTGCCGGCTGAAGAAGCGGCCCCCCGTGCGG |
| 215 | Exemplary | GCAGCATTCAAACCAAACCCAATCAACTACATCCTAGGACTAGACA |
| | coding sequence | TCGGAATCGCATCAGTAGGATGAGCAATGGTAGAAATCGACGAAG |
| | encoding | AAGAAAACCCAATCCGACTAATCGACCTAGGAGTACGAGTATTCGA |
| | Nme3Cas9 | ACGAGCAGAAGTACCAAAAACAGGAGACTCACTAGCAATGGCACG |
| | cleavase | ACGACTAGCACGATCAGTACGACGACTAACACGACGACGAGCACA |
| | | CCGACTACTACGAGCACGACGACTACTAAAACGAGAAGGAGTACT |
| | | ACAAGCAGCAGACTTCGACGAAAACGGACTAATCAAATCACTACC |
| | | AAACACACCATGACAACTACGAGCAGCAGCACTAGACCGAAAACT |
| | | AACACCACTAGAATGATCAGCAGTACTACTACACCTAATCAAACAC |
| | | CGAGGATACCTATCACAACGAAAAAACGAAGGAGAAACAGCAGAC |
| | | AAAGAACTAGGAGCACTACTAAAAGGAGTAGCAGACAACGCACAC |
| | | GCACTACAAACAGGAGACTTCCGAACACCAGCAGAACTAGCACTA |
| | | AACAAATTCGAAAAAGAATGCGGACACATCCGAAACCAACGAGGA |
| | | GACTACTCACACACATTCTCACGAAAAGACCTACAAGCAGAACTAA |
| | | ACCTACTATTCGAAAAACAAAAAGAATTCGGAAACCCACACGTATC |
| | | AGGAGGACTAAAAGAAGGAATCGAAACACTACTAATGACACAACG |
| | | ACCAGCACTATCAGGAGACGCAGTACAAAAAATGCTAGGACACTG |
| | | CACATTCGAACCAGCAGAACCAAAAGCAGCAAAAAACACATACAC |
| | | AGCAGAACGATTCATCTGACTAACAAAACTAAACAACCTACGAATC |
| | | CTAGAACAAGGATCAGAACGACCACTAACAGACACAGAACGAGCA |
| | | ACACTAATGGACGAACCATACCGAAAATCAAAACTAACATACGCA |
| | | CAAGCACGAAAACTACTATCACTAGAAGACACAGCATTCTTCAAAG |
| | | GACTACGATACGGAAAAGACAACGCAGAAGCATCAACACTAATGG |
| | | AAATGAAAGCATACCACACAATCTCACGAGCACTAGAAAAAGAAG |
| | | GACTAAAAGACAAAAAATCACCACTAAACCTATCACCAGAACTAC |
| | | AAGACGAAATCGGAACAGCATTCTCACTATTCAAAACAGACGAAG |
| | | ACATCACAGGACGACTAAAAGACCGAATCCAACCAGAAATCCTAG |
| | | AAGCACTACTAAAACACATCTCATTCGACAAATTCGTACAAATCTC |
| | | ACTAAAAGCACTACGACGAATCGTACCACTAATGGAACAAGGAAA |
| | | ACGATACGACGAAGCATGCGCAGAAATCTACGGAGACCACTACGG |
| | | AAAAAAAAACACAGAAGAAAAAATCTACCTACCACCAATCCCAGC |
| | | AGACGAAATCCGAAACCCAGTAGTACTACGAGCACTATCACAAGC |
| | | ACGAAAAGTAATCAACGGAGTAGTACGACGATACGGATCACCAGC |
| | | ACGAATCCACATCGAAACAGCACGAGAAGTAGGAAAATCATTCAA |
| | | AGACCGAAAAGAAATCGAAAAACGACAAGAAGAAAACCGAAAAG |
| | | ACCGAGAAAAAGCAGCAGCAAAATTCCGAGAATACTTCCCAAACTT |
| | | CGTAGGAGAACCAAAATCAAAAGACATCCTAAAACTACGACTATA |
| | | CGAACAACAACACGGAAAATGCCTATACTCAGGAAAAGAAATCAA |
| | | CCTAGGACGACTAAACGAAAAAGGATACGTAGAAATCGACCACGC |
| | | ACTACCATTCTCACGAACATGAGACGACTCATTCAACAACAAAGTA |
| | | CTAGTACTAGGATCAGAAAACCAAAACAAAGGAAACCAAACACCA |
| | | TACGAATACTTCAACGGAAAAGACAACTCACGAGAATGACAAGAA |
| | | TTCAAAGCACGAGTAGAAACATCACGATTCCCACGATCAAAAAAAC |
| | | AACGAATCCTACTACAAAAATTCGACGAAGACGGATTCAAAGAAC |
| | | GAAACCTAAACGACACACGATACGTAAACCGATTCCTATGCCAATT |
| | | CGTAGCAGACCGAATGCGACTAACAGGAAAAGGAAAAAAACGAGT |
| | | ATTCGCATCAAACGGACAAATCACAAACCTACTACGAGGATTCTGA |
| | | GGACTACGAAAAGTACGAGCAGAAAACGACCGACACCACGCACTA |
| | | GACGCAGTAGTAGTAGCATGCTCAACAGTAGCAATGCAACAAAAA |
| | | ATCACACGATTCGTACGATACAAAGAAATGAACGCATTCGACGGAA |
| | | AAACAATCGACAAAGAAACAGGAGAAGTACTACACCAAAAAACAC |
| | | ACTTCCCACAACCATGAGAATTCTTCGCACAAGAAGTAATGATCCG |
| | | AGTATTCGGAAAACCAGACGGAAAACCAGAATTCGAAGAAGCAGA |
| | | CACACCAGAAAAACTACGAACACTACTAGCAGAAAAACTATCATC |
| | | ACGACCAGAAGCAGTACACGAATACGTAACACCACTATTCGTATCA |
| | | CGAGCACCAAACCGAAAAATGTCAGGACAAGGACACATGGAAACA |
| | | GTAAAATCAGCAAAACGACTAGACGAAGGAGTATCAGTACTACGA |
| | | GTACCACTAACACAACTAAAACTAAAAGACCTAGAAAAAATGGTA |
| | | AACCGAGAACGAGAACCAAAACTATACGAAGCACTAAAAGCACGA |
| | | CTAGAAGCACACAAAGACGACCCAGCAAAAGCATTCGCAGAACCA |
| | | TTCTACAAATACGACAAAGCAGGAAACCGAACACAACAAGTAAAA |
| | | GCAGTACGAGTAGAACAAGTACAAAAAACAGGAGTATGAGTACGA |
| | | AACCACAACGGAATCGCAGACAACGCAACAATGGTACGAGTAGAC |
| | | GTATTCGAAAAAGGAGACAAATACTACCTAGTACCAATCTACTCAT |
| | | GACAAGTAGCAAAAGGAATCCTACCAGACCGAGCAGTAGTAGCAT |
| | | ACGCAGACGAAGAAGACTGAACAGTAATCGACGAATCATTCCGATT |
| | | CAAATTCGTACTATACTCAAACGACCTAATCAAAGTACAACTAAAA |
| | | AAAGACTCATTCCTAGGATACTTCTCAGGACTAGACCGAGCAACAG |
| | | GAGCAATCTCACTACGAGAACACGACCTAGAAAAATCAAAAGGAA |
| | | AAGACGGAATGCACCGAATCGGAGTAAAAACAGCACTATCATTCC |
| | | AAAAATACCAAATCGACGAAATGGGAAAAGAAATCCGACCATGCC |
| | | GACTAAAAAAACGACCACCAGTACGA |
| 216 | Exemplary open | ATGGCTGCTTTTAAGCCTAATCCTATTAATTATATTCTTGGTCTTGAT |
| | reading frame for | ATTGGTATTGCTTCTGTTGGTTGGGCTATGGTTGAGATTGATGAGGA |
| | Nme3Cas9 | GGAGAATCCTATTCGTCTTATTGATCTTGGTGTTCGTGTTTTTGAGC |
| | cleavase | GTGCTGAGGTTCCTAAGACTGGTGATTCTCTTGCTATGGCTCGTCGT |
| | | CTTGCTCGTTCTGTTCGTCGTCTTACTCGTCGTCGTGCTCATCGTCTT |
| | | CTTCGTGCTCGTCGTCTTCTTAAGCGTGAGGGTGTTCTTCAGGCTGC |
| | | TGATTTTGATGAGAATGGTCTTATTAAGTCTCTTCCTAATACTCCTT |
| | | GGCAGCTTCGTGCTGCTGCTCTTGATCGTAAGCTTACTCCTCTTGAG |
| | | TGGTCTGCTGTTCTTCTTCATCTTATTAAGCATCGTGGTTATCTTTCT |
| | | CAGCGTAAGAATGAGGGTGAGACTGCTGATAAGGAGCTTGGTGCTC |
| | | TTCTTAAGGGTGTTGCTGATAATGCTCATGCTCTTCAGACTGGTGAT |
| | | TTTCGTACTCCTGCTGAGCTTGCTCTTAATAAGTTTGAGAAGGAGTG |
| | | TGGTCATATTCGTAATCAGCGTGGTGATTATTCTCATACTTTTTCTC |
| | | GTAAGGATCTTCAGGCTGAGCTTAATCTTCTTTTTGAGAAGCAGAA |
| | | GGAGTTTGGTAATCCTCATGTTTCTGGTGGTCTTAAGGAGGGTATTG |
| | | AGACTCTTCTTATGACTCAGCGTCCTGCTCTTTCTGGTGATGCTGTT |
| | | CAGAAGATGCTTGGTCATTGTACTTTTGAGCCTGCTGAGCCTAAGG |
| | | CTGCTAAGAATACTTATACTGCTGAGCGTTTTATTTGGCTTACTAAG |
| | | CTTAATAATCTTCGTATTCTTGAGCAGGGTTCTGAGCGTCCTCTTAC |
| | | TGATACTGAGCGTGCTACTCTTATGGATGAGCCTTATCGTAAGTCTA |
| | | AGCTTACTTATGCTCAGGCTCGTAAGCTTCTTTCTCTTGAGGATACT |
| | | GCTTTTTTTAAGGGTCTTCGTTATGGTAAGGATAATGCTGAGGCTTC |
| | | TACTCTTATGGAGATGAAGGCTTATCATACTATTTCTCGTGCTCTTG |
| | | AGAAGGAGGGTCTTAAGGATAAGAAGTCTCCTCTTAATCTTTCTCCT |
| | | GAGCTTCAGGATGAGATTGGTACTGCTTTTTCTCTTTTTAAGACTGA |
| | | TGAGGATATTACTGGTCGTCTTAAGGATCGTATTCAGCCTGAGATTC |
| | | TTGAGGCTCTTCTTAAGCATATTTCTTTTGATAAGTTTGTTCAGATTT |
| | | CTCTTAAGGCTCTTCGTCGTATTGTTCCTCTTATGGAGCAGGGTAAG |
| | | CGTTATGATGAGGCTTGTGCTGAGATTTATGGTGATCATTATGGTAA |
| | | GAAGAATACTGAGGAGAAGATTTATCTTCCTCCTATTCCTGCTGATG |
| | | AGATTCGTAATCCTGTTGTTCTTCGTGCTCTTTCTCAGGCTCGTAAG |
| | | GTTATTAATGGTGTTGTTCGTCGTTATGGTTCTCCTGCTCGTATTCAT |
| | | ATTGAGACTGCTCGTGAGGTTGGTAAGTCTTTTAAGGATCGTAAGG |
| | | AGATTGAGAAGCGTCAGGAGGAGAATCGTAAGGATCGTGAGAAGG |
| | | CTGCTGCTAAGTTTCGTGAGTATTTTCCTAATTTTGTTGGTGAGCCT |
| | | AAGTCTAAGGATATTCTTAAGCTTCGTCTTTATGAGCAGCAGCATG |
| | | GTAAGTGTCTTTATTCTGGTAAGGAGATTAATCTTGGTCGTCTTAAT |
| | | GAGAAGGGTTATGTTGAGATTGATCATGCTCTTCCTTTTTCTCGTAC |
| | | TTGGGATGATTCTTTTAATAATAAGGTTCTTGTTCTTGGTTCTGAGA |
| | | ATCAGAATAAGGGTAATCAGACTCCTTATGAGTATTTTAATGGTAA |
| | | GGATAATTCTCGTGAGTGGCAGGAGTTTAAGGCTCGTGTTGAGACT |
| | | TCTCGTTTTCCTCGTTCTAAGAAGCAGCGTATTCTTCTTCAGAAGTT |
| | | TGATGAGGATGGTTTTAAGGAGCGTAATCTTAATGATACTCGTTAT |
| | | GTTAATCGTTTTCTTTGTCAGTTTGTTGCTGATCGTATGCGTCTTACT |
| | | GGTAAGGGTAAGAAGCGTGTTTTTGCTTCTAATGGTCAGATTACTA |
| | | ATCTTCTTCGTGGTTTTTGGGGTCTTCGTAAGGTTCGTGCTGAGAAT |
| | | GATCGTCATCATGCTCTTGATGCTGTTGTTGTTGCTTGTTCTACTGTT |
| | | GCTATGCAGCAGAAGATTACTCGTTTTGTTCGTTATAAGGAGATGA |
| | | ATGCTTTTGATGGTAAGACTATTGATAAGGAGACTGGTGAGGTTCT |
| | | TCATCAGAAGACTCATTTTCCTCAGCCTTGGGAGTTTTTTGCTCAGG |
| | | AGGTTATGATTCGTGTTTTTGGTAAGCCTGATGGTAAGCCTGAGTTT |
| | | GAGGAGGCTGATACTCCTGAGAAGCTTCGTACTCTTCTTGCTGAGA |
| | | AGCTTTCTTCTCGTCCTGAGGCTGTTCATGAGTATGTTACTCCTCTTT |
| | | TTGTTTCTCGTGCTCCTAATCGTAAGATGTCTGGTCAGGGTCATATG |
| | | GAGACTGTTAAGTCTGCTAAGCGTCTTGATGAGGGTGTTTCTGTTCT |
| | | TCGTGTTCCTCTTACTCAGCTTAAGCTTAAGGATCTTGAGAAGATGG |
| | | TTAATCGTGAGCGTGAGCCTAAGCTTTATGAGGCTCTTAAGGCTCGT |
| | | CTTGAGGCTCATAAGGATGATCCTGCTAAGGCTTTTGCTGAGCCTTT |
| | | TTATAAGTATGATAAGGCTGGTAATCGTACTCAGCAGGTTAAGGCT |
| | | GTTCGTGTTGAGCAGGTTCAGAAGACTGGTGTTTGGGTTCGTAATC |
| | | ATAATGGTATTGCTGATAATGCTACTATGGTTCGTGTTGATGTTTTT |
| | | GAGAAGGGTGATAAGTATTATCTTGTTCCTATTTATTCTTGGCAGGT |
| | | TGCTAAGGGTATTCTTCCTGATCGTGCTGTTGTTGCTTATGCTGATG |
| | | AGGAGGATTGGACTGTTATTGATGAGTCTTTTCGTTTTAAGTTTGTT |
| | | CTTTATTCTAATGATCTTATTAAGGTTCAGCTTAAGAAGGATTCTTT |
| | | TCTTGGTTATTTTTCTGGTCTTGATCGTGCTACTGGTGCTATTTCTCT |
| | | TCGTGAGCATGATCTTGAGAAGTCTAAGGGTAAGGATGGTATGCAT |
| | | CGTATTGGTGTTAAGACTGCTCTTTCTTTTCAGAAGTATCAGATTGA |
| | | TGAGATGGGTAAGGAGATTCGTCCTTGTCGTCTTAAGAAGCGTCCT |
| | | CCTGTTCGTUGA |
| 217 | Exemplary open | ATGGCCGCCTTCAAGCCCAACCCCATCAACTACATCCTGGGCCTGG |
| | reading frame for | ACATCGGCATCGCCTCCGTGGGCTGGGCCATGGTGGAGATCGACGA |
| | Nme3Cas9 | GGAGGAGAACCCCATCCGGCTGATCGACCTGGGCGTGCGGGTGTTC |
| | cleavase | GAGCGGGCCGAGGTGCCCAAGACCGGCGACTCCCTGGCCATGGCCC |
| | | GGCGGCTGGCCCGGTCCGTGCGGCGGCTGACCCGGCGGCGGGCCCA |
| | | CCGGCTGCTGCGGGCCCGGCGGCTGCTGAAGCGGGAGGGCGTGCTG |
| | | CAGGCCGCCGACTTCGACGAGAACGGCCTGATCAAGTCCCTGCCCA |
| | | ACACCCCCTGGCAGCTGCGGGCCGCCGCCCTGGACCGGAAGCTGAC |
| | | CCCCCTGGAGTGGTCCGCCGTGCTGCTGCACCTGATCAAGCACCGG |
| | | GGCTACCTGTCCCAGCGGAAGAACGAGGGCGAGACCGCCGACAAG |
| | | GAGCTGGGCGCCCTGCTGAAGGGCGTGGCCGACAACGCCCACGCCC |
| | | TGCAGACCGGCGACTTCCGGACCCCCGCCGAGCTGGCCCTGAACAA |
| | | GTTCGAGAAGGAGTGCGGCCACATCCGGAACCAGCGGGGCGACTA |
| | | CTCCCACACCTTCTCCCGGAAGGACCTGCAGGCCGAGCTGAACCTG |
| | | CTGTTCGAGAAGCAGAAGGAGTTCGGCAACCCCCACGTGTCCGGCG |
| | | GCCTGAAGGAGGGCATCGAGACCCTGCTGATGACCCAGCGGCCCGC |
| | | CCTGTCCGGCGACGCCGTGCAGAAGATGCTGGGCCACTGCACCTTC |
| | | GAGCCCGCCGAGCCCAAGGCCGCCAAGAACACCTACACCGCCGAG |
| | | CGGTTCATCTGGCTGACCAAGCTGAACAACCTGCGGATCCTGGAGC |
| | | AGGGCTCCGAGCGGCCCCTGACCGACACCGAGCGGGCCACCCTGAT |
| | | GGACGAGCCCTACCGGAAGTCCAAGCTGACCTACGCCCAGGCCCGG |
| | | AAGCTGCTGTCCCTGGAGGACACCGCCTTCTTCAAGGGCCTGCGGT |
| | | ACGGCAAGGACAACGCCGAGGCCTCCACCCTGATGGAGATGAAGG |
| | | CCTACCACACCATCTCCCGGGCCCTGGAGAAGGAGGGCCTGAAGGA |
| | | CAAGAAGTCCCCCCTGAACCTGTCCCCCGAGCTGCAGGACGAGATC |
| | | GGCACCGCCTTCTCCCTGTTCAAGACCGACGAGGACATCACCGGCC |
| | | GGCTGAAGGACCGGATCCAGCCCGAGATCCTGGAGGCCCTGCTGAA |
| | | GCACATCTCCTTCGACAAGTTCGTGCAGATCTCCCTGAAGGCCCTGC |
| | | GGCGGATCGTGCCCCTGATGGAGCAGGGCAAGCGGTACGACGAGG |
| | | CCTGCGCCGAGATCTACGGCGACCACTACGGCAAGAAGAACACCG |
| | | AGGAGAAGATCTACCTGCCCCCCATCCCCGCCGACGAGATCCGGAA |
| | | CCCCGTGGTGCTGCGGGCCCTGTCCCAGGCCCGGAAGGTGATCAAC |
| | | GGCGTGGTGCGGCGGTACGGCTCCCCCGCCCGGATCCACATCGAGA |
| | | CCGCCCGGGAGGTGGGCAAGTCCTTCAAGGACCGGAAGGAGATCG |
| | | AGAAGCGGCAGGAGGAGAACCGGAAGGACCGGGAGAAGGCCGCC |
| | | GCCAAGTTCCGGGAGTACTTCCCCAACTTCGTGGGCGAGCCCAAGT |
| | | CCAAGGACATCCTGAAGCTGCGGCTGTACGAGCAGCAGCACGGCA |
| | | AGTGCCTGTACTCCGGCAAGGAGATCAACCTGGGCCGGCTGAACGA |
| | | GAAGGGCTACGTGGAGATCGACCACGCCCTGCCCTTCTCCCGGACC |
| | | TGGGACGACTCCTTCAACAACAAGGTGCTGGTGCTGGGCTCCGAGA |
| | | ACCAGAACAAGGGCAACCAGACCCCCTACGAGTACTTCAACGGCA |
| | | AGGACAACTCCCGGGAGTGGCAGGAGTTCAAGGCCCGGGTGGAGA |
| | | CCTCCCGGTTCCCCCGGTCCAAGAAGCAGCGGATCCTGCTGCAGAA |
| | | GTTCGACGAGGACGGCTTCAAGGAGCGGAACCTGAACGACACCCG |
| | | GTACGTGAACCGGTTCCTGTGCCAGTTCGTGGCCGACCGGATGCGG |
| | | CTGACCGGCAAGGGCAAGAAGCGGGTGTTCGCCTCCAACGGCCAG |
| | | ATCACCAACCTGCTGCGGGGCTTCTGGGGCCTGCGGAAGGTGCGGG |
| | | CCGAGAACGACCGGCACCACGCCCTGGACGCCGTGGTGGTGGCCTG |
| | | CTCCACCGTGGCCATGCAGCAGAAGATCACCCGGTTCGTGCGGTAC |
| | | AAGGAGATGAACGCCTTCGACGGCAAGACCATCGACAAGGAGACC |
| | | GGCGAGGTGCTGCACCAGAAGACCCACTTCCCCCAGCCCTGGGAGT |
| | | TCTTCGCCCAGGAGGTGATGATCCGGGTGTTCGGCAAGCCCGACGG |
| | | CAAGCCCGAGTTCGAGGAGGCCGACACCCCCGAGAAGCTGCGGAC |
| | | CCTGCTGGCCGAGAAGCTGTCCTCCCGGCCCGAGGCCGTGCACGAG |
| | | TACGTGACCCCCCTGTTCGTGTCCCGGGCCCCCAACCGGAAGATGT |
| | | CCGGCCAGGGCCACATGGAGACCGTGAAGTCCGCCAAGCGGCTGG |
| | | ACGAGGGCGTGTCCGTGCTGCGGGTGCCCCTGACCCAGCTGAAGCT |
| | | GAAGGACCTGGAGAAGATGGTGAACCGGGAGCGGGAGCCCAAGCT |
| | | GTACGAGGCCCTGAAGGCCCGGCTGGAGGCCCACAAGGACGACCC |
| | | CGCCAAGGCCTTCGCCGAGCCCTTCTACAAGTACGACAAGGCCGGC |
| | | AACCGGACCCAGCAGGTGAAGGCCGTGCGGGTGGAGCAGGTGCAG |
| | | AAGACCGGCGTGTGGGTGCGGAACCACAACGGCATCGCCGACAAC |
| | | GCCACCATGGTGCGGGTGGACGTGTTCGAGAAGGGCGACAAGTACT |
| | | ACCTGGTGCCCATCTACTCCTGGCAGGTGGCCAAGGGCATCCTGCC |
| | | CGACCGGGCCGTGGTGGCCTACGCCGACGAGGAGGACTGGACCGT |
| | | GATCGACGAGTCCTTCCGGTTCAAGTTCGTGCTGTACTCCAACGACC |
| | | TGATCAAGGTGCAGCTGAAGAAGGACTCCTTCCTGGGCTACTTCTC |
| | | CGGCCTGGACCGGGCCACCGGCGCCATCTCCCTGCGGGAGCACGAC |
| | | CTGGAGAAGTCCAAGGGCAAGGACGGCATGCACCGGATCGGCGTG |
| | | AAGACCGCCCTGTCCTTCCAGAAGTACCAGATCGACGAGATGGGCA |
| | | AGGAGATCCGGCCCTGCCGGCTGAAGAAGCGGCCCCCCGTGCGGU |
| | | GA |
| 218 | Exemplary open | ATGGCAGCATTCAAACCAAACCCAATCAACTACATCCTAGGACTAG |
| | reading frame for | ACATCGGAATCGCATCAGTAGGATGAGCAATGGTAGAAATCGACG |
| | Nme3Cas9 | AAGAAGAAAACCCAATCCGACTAATCGACCTAGGAGTACGAGTATT |
| | cleavase | CGAACGAGCAGAAGTACCAAAAACAGGAGACTCACTAGCAATGGC |
| | | ACGACGACTAGCACGATCAGTACGACGACTAACACGACGACGAGC |
| | | ACACCGACTACTACGAGCACGACGACTACTAAAACGAGAAGGAGT |
| | | ACTACAAGCAGCAGACTTCGACGAAAACGGACTAATCAAATCACTA |
| | | CCAAACACACCATGACAACTACGAGCAGCAGCACTAGACCGAAAA |
| | | CTAACACCACTAGAATGATCAGCAGTACTACTACACCTAATCAAAC |
| | | ACCGAGGATACCTATCACAACGAAAAAACGAAGGAGAAACAGCAG |
| | | ACAAAGAACTAGGAGCACTACTAAAAGGAGTAGCAGACAACGCAC |
| | | ACGCACTACAAACAGGAGACTTCCGAACACCAGCAGAACTAGCAC |
| | | TAAACAAATTCGAAAAAGAATGCGGACACATCCGAAACCAACGAG |
| | | GAGACTACTCACACACATTCTCACGAAAAGACCTACAAGCAGAACT |
| | | AAACCTACTATTCGAAAAACAAAAAGAATTCGGAAACCCACACGT |
| | | ATCAGGAGGACTAAAAGAAGGAATCGAAACACTACTAATGACACA |
| | | ACGACCAGCACTATCAGGAGACGCAGTACAAAAAATGCTAGGACA |
| | | CTGCACATTCGAACCAGCAGAACCAAAAGCAGCAAAAAACACATA |
| | | CACAGCAGAACGATTCATCTGACTAACAAAACTAAACAACCTACGA |
| | | ATCCTAGAACAAGGATCAGAACGACCACTAACAGACACAGAACGA |
| | | GCAACACTAATGGACGAACCATACCGAAAATCAAAACTAACATAC |
| | | GCACAAGCACGAAAACTACTATCACTAGAAGACACAGCATTCTTCA |
| | | AAGGACTACGATACGGAAAAGACAACGCAGAAGCATCAACACTAA |
| | | TGGAAATGAAAGCATACCACACAATCTCACGAGCACTAGAAAAAG |
| | | AAGGACTAAAAGACAAAAAATCACCACTAAACCTATCACCAGAAC |
| | | TACAAGACGAAATCGGAACAGCATTCTCACTATTCAAAACAGACGA |
| | | AGACATCACAGGACGACTAAAAGACCGAATCCAACCAGAAATCCT |
| | | AGAAGCACTACTAAAACACATCTCATTCGACAAATTCGTACAAATC |
| | | TCACTAAAAGCACTACGACGAATCGTACCACTAATGGAACAAGGA |
| | | AAACGATACGACGAAGCATGCGCAGAAATCTACGGAGACCACTAC |
| | | GGAAAAAAAAACACAGAAGAAAAAATCTACCTACCACCAATCCCA |
| | | GCAGACGAAATCCGAAACCCAGTAGTACTACGAGCACTATCACAA |
| | | GCACGAAAAGTAATCAACGGAGTAGTACGACGATACGGATCACCA |
| | | GCACGAATCCACATCGAAACAGCACGAGAAGTAGGAAAATCATTC |
| | | AAAGACCGAAAAGAAATCGAAAAACGACAAGAAGAAAACCGAAA |
| | | AGACCGAGAAAAAGCAGCAGCAAAATTCCGAGAATACTTCCCAAA |
| | | CTTCGTAGGAGAACCAAAATCAAAAGACATCCTAAAACTACGACTA |
| | | TACGAACAACAACACGGAAAATGCCTATACTCAGGAAAAGAAATC |
| | | AACCTAGGACGACTAAACGAAAAAGGATACGTAGAAATCGACCAC |
| | | GCACTACCATTCTCACGAACATGAGACGACTCATTCAACAACAAAG |
| | | TACTAGTACTAGGATCAGAAAACCAAAACAAAGGAAACCAAACAC |
| | | CATACGAATACTTCAACGGAAAAGACAACTCACGAGAATGACAAG |
| | | AATTCAAAGCACGAGTAGAAACATCACGATTCCCACGATCAAAAA |
| | | AACAACGAATCCTACTACAAAAATTCGACGAAGACGGATTCAAAG |
| | | AACGAAACCTAAACGACACACGATACGTAAACCGATTCCTATGCCA |
| | | ATTCGTAGCAGACCGAATGCGACTAACAGGAAAAGGAAAAAAACG |
| | | AGTATTCGCATCAAACGGACAAATCACAAACCTACTACGAGGATTC |
| | | TGAGGACTACGAAAAGTACGAGCAGAAAACGACCGACACCACGCA |
| | | CTAGACGCAGTAGTAGTAGCATGCTCAACAGTAGCAATGCAACAAA |
| | | AAATCACACGATTCGTACGATACAAAGAAATGAACGCATTCGACGG |
| | | AAAAACAATCGACAAAGAAACAGGAGAAGTACTACACCAAAAAAC |
| | | ACACTTCCCACAACCATGAGAATTCTTCGCACAAGAAGTAATGATC |
| | | CGAGTATTCGGAAAACCAGACGGAAAACCAGAATTCGAAGAAGCA |
| | | GACACACCAGAAAAACTACGAACACTACTAGCAGAAAAACTATCA |
| | | TCACGACCAGAAGCAGTACACGAATACGTAACACCACTATTCGTAT |
| | | CACGAGCACCAAACCGAAAAATGTCAGGACAAGGACACATGGAAA |
| | | CAGTAAAATCAGCAAAACGACTAGACGAAGGAGTATCAGTACTAC |
| | | GAGTACCACTAACACAACTAAAACTAAAAGACCTAGAAAAAATGG |
| | | TAAACCGAGAACGAGAACCAAAACTATACGAAGCACTAAAAGCAC |
| | | GACTAGAAGCACACAAAGACGACCCAGCAAAAGCATTCGCAGAAC |
| | | CATTCTACAAATACGACAAAGCAGGAAACCGAACACAACAAGTAA |
| | | AAGCAGTACGAGTAGAACAAGTACAAAAAACAGGAGTATGAGTAC |
| | | GAAACCACAACGGAATCGCAGACAACGCAACAATGGTACGAGTAG |
| | | ACGTATTCGAAAAAGGAGACAAATACTACCTAGTACCAATCTACTC |
| | | ATGACAAGTAGCAAAAGGAATCCTACCAGACCGAGCAGTAGTAGC |
| | | ATACGCAGACGAAGAAGACTGAACAGTAATCGACGAATCATTCCG |
| | | ATTCAAATTCGTACTATACTCAAACGACCTAATCAAAGTACAACTA |
| | | AAAAAAGACTCATTCCTAGGATACTTCTCAGGACTAGACCGAGCAA |
| | | CAGGAGCAATCTCACTACGAGAACACGACCTAGAAAAATCAAAAG |
| | | GAAAAGACGGAATGCACCGAATCGGAGTAAAAACAGCACTATCAT |
| | | TCCAAAAATACCAAATCGACGAAATGGGAAAAGAAATCCGACCAT |
| | | GCCGACTAAAAAAACGACCACCAGTACGAUAA |
| 219 | Exemplary amino | MAAFKPNPINYILGLDIGIASVGWAMVEIDEEENPIRLIDLGVRVFERAE |
| | acid sequence of | VPKTGDSLAMARRLARSVRRLTRRRAHRLLRARRLLKREGVLQAADF |
| | Nme3Cas9 HNH | DENGLIKSLPNTPWQLRAAALDRKLTPLEWSAVLLHLIKHRGYLSQRK |
| | nickase | NEGETADKELGALLKGVADNAHALQTGDFRTPAELALNKFEKECGHI |
| | | RNQRGDYSHTFSRKDLQAELNLLFEKQKEFGNPHVSGGLKEGIETLLM |
| | | TQRPALSGDAVQKMLGHCTFEPAEPKAAKNTYTAERFIWLTKLNNLRI |
| | | LEQGSERPLTDTERATLMDEPYRKSKLTYAQARKLLSLEDTAFFKGLR |
| | | YGKDNAEASTLMEMKAYHTISRALEKEGLKDKKSPLNLSPELQDEIGT |
| | | AFSLFKTDEDITGRLKDRIQPEILEALLKHISFDKFVQISLKALRRIVPLM |
| | | EQGKRYDEACAEIYGDHYGKKNTEEKIYLPPIPADEIRNPVVLRALSQA |
| | | RKVINGVVRRYGSPARIHIETAREVGKSFKDRKEIEKRQEENRKDREKA |
| | | AAKFREYFPNFVGEPKSKDILKLRLYEQQHGKCLYSGKEINLGRLNEK |
| | | GYVEIDAALPFSRTWDDSFNNKVLVLGSENQNKGNQTPYEYFNGKDN |
| | | SREWQEFKARVETSRFPRSKKQRILLQKFDEDGFKERNLNDTRYVNRF |
| | | LCQFVADRMRLTGKGKKRVFASNGQITNLLRGFWGLRKVRAENDRH |
| | | HALDAVVVACSTVAMQQKITRFVRYKEMNAFDGKTIDKETGEVLHQ |
| | | KTHFPQPWEFFAQEVMIRVFGKPDGKPEFEEADTPEKLRTLLAEKLSSR |
| | | PEAVHEYVTPLFVSRAPNRKMSGQGHMETVKSAKRLDEGVSVLRVPL |
| | | TQLKLKDLEKMVNREREPKLYEALKARLEAHKDDPAKAFAEPFYKYD |
| | | KAGNRTQQVKAVRVEQVQKTGVWVRNHNGIADNATMVRVDVFEKG |
| | | DKYYLVPIYSWQVAKGILPDRAVVAYADEEDWTVIDESFRFKFVLYSN |
| | | DLIKVQLKKDSFLGYFSGLDRATGAISLREHDLEKSKGKDGMHRIGVK |
| | | TALSFQKYQIDEMGKEIRPCRLKKRPPVR |
| 220 | Exemplary | GCTGCTTTTAAGCCTAATCCTATTAATTATATTCTTGGTCTTGATATT |
| | coding sequence | GGTATTGCTTCTGTTGGTTGGGCTATGGTTGAGATTGATGAGGAGG |
| | encoding | AGAATCCTATTCGTCTTATTGATCTTGGTGTTCGTGTTTTTGAGCGT |
| | Nme3Cas9 HNH | GCTGAGGTTCCTAAGACTGGTGATTCTCTTGCTATGGCTCGTCGTCT |
| | nickase | TGCTCGTTCTGTTCGTCGTCTTACTCGTCGTCGTGCTCATCGTCTTCT |
| | | TCGTGCTCGTCGTCTTCTTAAGCGTGAGGGTGTTCTTCAGGCTGCTG |
| | | ATTTTGATGAGAATGGTCTTATTAAGTCTCTTCCTAATACTCCTTGG |
| | | CAGCTTCGTGCTGCTGCTCTTGATCGTAAGCTTACTCCTCTTGAGTG |
| | | GTCTGCTGTTCTTCTTCATCTTATTAAGCATCGTGGTTATCTTTCTCA |
| | | GCGTAAGAATGAGGGTGAGACTGCTGATAAGGAGCTTGGTGCTCTT |
| | | CTTAAGGGTGTTGCTGATAATGCTCATGCTCTTCAGACTGGTGATTT |
| | | TCGTACTCCTGCTGAGCTTGCTCTTAATAAGTTTGAGAAGGAGTGTG |
| | | GTCATATTCGTAATCAGCGTGGTGATTATTCTCATACTTTTTCTCGT |
| | | AAGGATCTTCAGGCTGAGCTTAATCTTCTTTTTGAGAAGCAGAAGG |
| | | AGTTTGGTAATCCTCATGTTTCTGGTGGTCTTAAGGAGGGTATTGAG |
| | | ACTCTTCTTATGACTCAGCGTCCTGCTCTTTCTGGTGATGCTGTTCA |
| | | GAAGATGCTTGGTCATTGTACTTTTGAGCCTGCTGAGCCTAAGGCTG |
| | | CTAAGAATACTTATACTGCTGAGCGTTTTATTTGGCTTACTAAGCTT |
| | | AATAATCTTCGTATTCTTGAGCAGGGTTCTGAGCGTCCTCTTACTGA |
| | | TACTGAGCGTGCTACTCTTATGGATGAGCCTTATCGTAAGTCTAAGC |
| | | TTACTTATGCTCAGGCTCGTAAGCTTCTTTCTCTTGAGGATACTGCT |
| | | TTTTTTAAGGGTCTTCGTTATGGTAAGGATAATGCTGAGGCTTCTAC |
| | | TCTTATGGAGATGAAGGCTTATCATACTATTTCTCGTGCTCTTGAGA |
| | | AGGAGGGTCTTAAGGATAAGAAGTCTCCTCTTAATCTTTCTCCTGAG |
| | | CTTCAGGATGAGATTGGTACTGCTTTTTCTCTTTTTAAGACTGATGA |
| | | GGATATTACTGGTCGTCTTAAGGATCGTATTCAGCCTGAGATTCTTG |
| | | AGGCTCTTCTTAAGCATATTTCTTTTGATAAGTTTGTTCAGATTTCTC |
| | | TTAAGGCTCTTCGTCGTATTGTTCCTCTTATGGAGCAGGGTAAGCGT |
| | | TATGATGAGGCTTGTGCTGAGATTTATGGTGATCATTATGGTAAGA |
| | | AGAATACTGAGGAGAAGATTTATCTTCCTCCTATTCCTGCTGATGAG |
| | | ATTCGTAATCCTGTTGTTCTTCGTGCTCTTTCTCAGGCTCGTAAGGTT |
| | | ATTAATGGTGTTGTTCGTCGTTATGGTTCTCCTGCTCGTATTCATATT |
| | | GAGACTGCTCGTGAGGTTGGTAAGTCTTTTAAGGATCGTAAGGAGA |
| | | TTGAGAAGCGTCAGGAGGAGAATCGTAAGGATCGTGAGAAGGCTG |
| | | CTGCTAAGTTTCGTGAGTATTTTCCTAATTTTGTTGGTGAGCCTAAG |
| | | TCTAAGGATATTCTTAAGCTTCGTCTTTATGAGCAGCAGCATGGTAA |
| | | GTGTCTTTATTCTGGTAAGGAGATTAATCTTGGTCGTCTTAATGAGA |
| | | AGGGTTATGTTGAGATTGATGCTGCTCTTCCTTTTTCTCGTACTTGG |
| | | GATGATTCTTTTAATAATAAGGTTCTTGTTCTTGGTTCTGAGAATCA |
| | | GAATAAGGGTAATCAGACTCCTTATGAGTATTTTAATGGTAAGGAT |
| | | AATTCTCGTGAGTGGCAGGAGTTTAAGGCTCGTGTTGAGACTTCTC |
| | | GTTTTCCTCGTTCTAAGAAGCAGCGTATTCTTCTTCAGAAGTTTGAT |
| | | GAGGATGGTTTTAAGGAGCGTAATCTTAATGATACTCGTTATGTTA |
| | | ATCGTTTTCTTTGTCAGTTTGTTGCTGATCGTATGCGTCTTACTGGTA |
| | | AGGGTAAGAAGCGTGTTTTTGCTTCTAATGGTCAGATTACTAATCTT |
| | | CTTCGTGGTTTTTGGGGTCTTCGTAAGGTTCGTGCTGAGAATGATCG |
| | | TCATCATGCTCTTGATGCTGTTGTTGTTGCTTGTTCTACTGTTGCTAT |
| | | GCAGCAGAAGATTACTCGTTTTGTTCGTTATAAGGAGATGAATGCT |
| | | TTTGATGGTAAGACTATTGATAAGGAGACTGGTGAGGTTCTTCATC |
| | | AGAAGACTCATTTTCCTCAGCCTTGGGAGTTTTTTGCTCAGGAGGTT |
| | | ATGATTCGTGTTTTTGGTAAGCCTGATGGTAAGCCTGAGTTTGAGGA |
| | | GGCTGATACTCCTGAGAAGCTTCGTACTCTTCTTGCTGAGAAGCTTT |
| | | CTTCTCGTCCTGAGGCTGTTCATGAGTATGTTACTCCTCTTTTTGTTT |
| | | CTCGTGCTCCTAATCGTAAGATGTCTGGTCAGGGTCATATGGAGAC |
| | | TGTTAAGTCTGCTAAGCGTCTTGATGAGGGTGTTTCTGTTCTTCGTG |
| | | TTCCTCTTACTCAGCTTAAGCTTAAGGATCTTGAGAAGATGGTTAAT |
| | | CGTGAGCGTGAGCCTAAGCTTTATGAGGCTCTTAAGGCTCGTCTTG |
| | | AGGCTCATAAGGATGATCCTGCTAAGGCTTTTGCTGAGCCTTTTTAT |
| | | AAGTATGATAAGGCTGGTAATCGTACTCAGCAGGTTAAGGCTGTTC |
| | | GTGTTGAGCAGGTTCAGAAGACTGGTGTTTGGGTTCGTAATCATAA |
| | | TGGTATTGCTGATAATGCTACTATGGTTCGTGTTGATGTTTTTGAGA |
| | | AGGGTGATAAGTATTATCTTGTTCCTATTTATTCTTGGCAGGTTGCT |
| | | AAGGGTATTCTTCCTGATCGTGCTGTTGTTGCTTATGCTGATGAGGA |
| | | GGATTGGACTGTTATTGATGAGTCTTTTCGTTTTAAGTTTGTTCTTTA |
| | | TTCTAATGATCTTATTAAGGTTCAGCTTAAGAAGGATTCTTTTCTTG |
| | | GTTATTTTTCTGGTCTTGATCGTGCTACTGGTGCTATTTCTCTTCGTG |
| | | AGCATGATCTTGAGAAGTCTAAGGGTAAGGATGGTATGCATCGTAT |
| | | TGGTGTTAAGACTGCTCTTTCTTTTCAGAAGTATCAGATTGATGAGA |
| | | TGGGTAAGGAGATTCGTCCTTGTCGTCTTAAGAAGCGTCCTCCTGTT |
| | | CGT |
| 221 | Exemplary | GCCGCCTTCAAGCCCAACCCCATCAACTACATCCTGGGCCTGGACA |
| | coding sequence | TCGGCATCGCCTCCGTGGGCTGGGCCATGGTGGAGATCGACGAGGA |
| | encoding | GGAGAACCCCATCCGGCTGATCGACCTGGGCGTGCGGGTGTTCGAG |
| | Nme3Cas9 HNH | CGGGCCGAGGTGCCCAAGACCGGCGACTCCCTGGCCATGGCCCGGC |
| | nickase | GGCTGGCCCGGTCCGTGCGGCGGCTGACCCGGCGGCGGGCCCACCG |
| | | GCTGCTGCGGGCCCGGCGGCTGCTGAAGCGGGAGGGCGTGCTGCA |
| | | GGCCGCCGACTTCGACGAGAACGGCCTGATCAAGTCCCTGCCCAAC |
| | | ACCCCCTGGCAGCTGCGGGCCGCCGCCCTGGACCGGAAGCTGACCC |
| | | CCCTGGAGTGGTCCGCCGTGCTGCTGCACCTGATCAAGCACCGGGG |
| | | CTACCTGTCCCAGCGGAAGAACGAGGGCGAGACCGCCGACAAGGA |
| | | GCTGGGCGCCCTGCTGAAGGGCGTGGCCGACAACGCCCACGCCCTG |
| | | CAGACCGGCGACTTCCGGACCCCCGCCGAGCTGGCCCTGAACAAGT |
| | | TCGAGAAGGAGTGCGGCCACATCCGGAACCAGCGGGGCGACTACT |
| | | CCCACACCTTCTCCCGGAAGGACCTGCAGGCCGAGCTGAACCTGCT |
| | | GTTCGAGAAGCAGAAGGAGTTCGGCAACCCCCACGTGTCCGGCGGC |
| | | CTGAAGGAGGGCATCGAGACCCTGCTGATGACCCAGCGGCCCGCCC |
| | | TGTCCGGCGACGCCGTGCAGAAGATGCTGGGCCACTGCACCTTCGA |
| | | GCCCGCCGAGCCCAAGGCCGCCAAGAACACCTACACCGCCGAGCG |
| | | GTTCATCTGGCTGACCAAGCTGAACAACCTGCGGATCCTGGAGCAG |
| | | GGCTCCGAGCGGCCCCTGACCGACACCGAGCGGGCCACCCTGATGG |
| | | ACGAGCCCTACCGGAAGTCCAAGCTGACCTACGCCCAGGCCCGGAA |
| | | GCTGCTGTCCCTGGAGGACACCGCCTTCTTCAAGGGCCTGCGGTAC |
| | | GGCAAGGACAACGCCGAGGCCTCCACCCTGATGGAGATGAAGGCC |
| | | TACCACACCATCTCCCGGGCCCTGGAGAAGGAGGGCCTGAAGGAC |
| | | AAGAAGTCCCCCCTGAACCTGTCCCCCGAGCTGCAGGACGAGATCG |
| | | GCACCGCCTTCTCCCTGTTCAAGACCGACGAGGACATCACCGGCCG |
| | | GCTGAAGGACCGGATCCAGCCCGAGATCCTGGAGGCCCTGCTGAAG |
| | | CACATCTCCTTCGACAAGTTCGTGCAGATCTCCCTGAAGGCCCTGCG |
| | | GCGGATCGTGCCCCTGATGGAGCAGGGCAAGCGGTACGACGAGGC |
| | | CTGCGCCGAGATCTACGGCGACCACTACGGCAAGAAGAACACCGA |
| | | GGAGAAGATCTACCTGCCCCCCATCCCCGCCGACGAGATCCGGAAC |
| | | CCCGTGGTGCTGCGGGCCCTGTCCCAGGCCCGGAAGGTGATCAACG |
| | | GCGTGGTGCGGCGGTACGGCTCCCCCGCCCGGATCCACATCGAGAC |
| | | CGCCCGGGAGGTGGGCAAGTCCTTCAAGGACCGGAAGGAGATCGA |
| | | GAAGCGGCAGGAGGAGAACCGGAAGGACCGGGAGAAGGCCGCCG |
| | | CCAAGTTCCGGGAGTACTTCCCCAACTTCGTGGGCGAGCCCAAGTC |
| | | CAAGGACATCCTGAAGCTGCGGCTGTACGAGCAGCAGCACGGCAA |
| | | GTGCCTGTACTCCGGCAAGGAGATCAACCTGGGCCGGCTGAACGAG |
| | | AAGGGCTACGTGGAGATCGACGCCGCCCTGCCCTTCTCCCGGACCT |
| | | GGGACGACTCCTTCAACAACAAGGTGCTGGTGCTGGGCTCCGAGAA |
| | | CCAGAACAAGGGCAACCAGACCCCCTACGAGTACTTCAACGGCAA |
| | | GGACAACTCCCGGGAGTGGCAGGAGTTCAAGGCCCGGGTGGAGAC |
| | | CTCCCGGTTCCCCCGGTCCAAGAAGCAGCGGATCCTGCTGCAGAAG |
| | | TTCGACGAGGACGGCTTCAAGGAGCGGAACCTGAACGACACCCGG |
| | | TACGTGAACCGGTTCCTGTGCCAGTTCGTGGCCGACCGGATGCGGC |
| | | TGACCGGCAAGGGCAAGAAGCGGGTGTTCGCCTCCAACGGCCAGA |
| | | TCACCAACCTGCTGCGGGGCTTCTGGGGCCTGCGGAAGGTGCGGGC |
| | | CGAGAACGACCGGCACCACGCCCTGGACGCCGTGGTGGTGGCCTGC |
| | | TCCACCGTGGCCATGCAGCAGAAGATCACCCGGTTCGTGCGGTACA |
| | | AGGAGATGAACGCCTTCGACGGCAAGACCATCGACAAGGAGACCG |
| | | GCGAGGTGCTGCACCAGAAGACCCACTTCCCCCAGCCCTGGGAGTT |
| | | CTTCGCCCAGGAGGTGATGATCCGGGTGTTCGGCAAGCCCGACGGC |
| | | AAGCCCGAGTTCGAGGAGGCCGACACCCCCGAGAAGCTGCGGACC |
| | | CTGCTGGCCGAGAAGCTGTCCTCCCGGCCCGAGGCCGTGCACGAGT |
| | | ACGTGACCCCCCTGTTCGTGTCCCGGGCCCCCAACCGGAAGATGTC |
| | | CGGCCAGGGCCACATGGAGACCGTGAAGTCCGCCAAGCGGCTGGA |
| | | CGAGGGCGTGTCCGTGCTGCGGGTGCCCCTGACCCAGCTGAAGCTG |
| | | AAGGACCTGGAGAAGATGGTGAACCGGGAGCGGGAGCCCAAGCTG |
| | | TACGAGGCCCTGAAGGCCCGGCTGGAGGCCCACAAGGACGACCCC |
| | | GCCAAGGCCTTCGCCGAGCCCTTCTACAAGTACGACAAGGCCGGCA |
| | | ACCGGACCCAGCAGGTGAAGGCCGTGCGGGTGGAGCAGGTGCAGA |
| | | AGACCGGCGTGTGGGTGCGGAACCACAACGGCATCGCCGACAACG |
| | | CCACCATGGTGCGGGTGGACGTGTTCGAGAAGGGCGACAAGTACTA |
| | | CCTGGTGCCCATCTACTCCTGGCAGGTGGCCAAGGGCATCCTGCCC |
| | | GACCGGGCCGTGGTGGCCTACGCCGACGAGGAGGACTGGACCGTG |
| | | ATCGACGAGTCCTTCCGGTTCAAGTTCGTGCTGTACTCCAACGACCT |
| | | GATCAAGGTGCAGCTGAAGAAGGACTCCTTCCTGGGCTACTTCTCC |
| | | GGCCTGGACCGGGCCACCGGCGCCATCTCCCTGCGGGAGCACGACC |
| | | TGGAGAAGTCCAAGGGCAAGGACGGCATGCACCGGATCGGCGTGA |
| | | AGACCGCCCTGTCCTTCCAGAAGTACCAGATCGACGAGATGGGCAA |
| | | GGAGATCCGGCCCTGCCGGCTGAAGAAGCGGCCCCCCGTGCGG |
| 222 | Exemplary | GCAGCATTCAAACCAAACCCAATCAACTACATCCTAGGACTAGACA |
| | coding sequence | TCGGAATCGCATCAGTAGGATGAGCAATGGTAGAAATCGACGAAG |
| | encoding | AAGAAAACCCAATCCGACTAATCGACCTAGGAGTACGAGTATTCGA |
| | Nme3Cas9 HNH | ACGAGCAGAAGTACCAAAAACAGGAGACTCACTAGCAATGGCACG |
| | nickase | ACGACTAGCACGATCAGTACGACGACTAACACGACGACGAGCACA |
| | | CCGACTACTACGAGCACGACGACTACTAAAACGAGAAGGAGTACT |
| | | ACAAGCAGCAGACTTCGACGAAAACGGACTAATCAAATCACTACC |
| | | AAACACACCATGACAACTACGAGCAGCAGCACTAGACCGAAAACT |
| | | AACACCACTAGAATGATCAGCAGTACTACTACACCTAATCAAACAC |
| | | CGAGGATACCTATCACAACGAAAAAACGAAGGAGAAACAGCAGAC |
| | | AAAGAACTAGGAGCACTACTAAAAGGAGTAGCAGACAACGCACAC |
| | | GCACTACAAACAGGAGACTTCCGAACACCAGCAGAACTAGCACTA |
| | | AACAAATTCGAAAAAGAATGCGGACACATCCGAAACCAACGAGGA |
| | | GACTACTCACACACATTCTCACGAAAAGACCTACAAGCAGAACTAA |
| | | ACCTACTATTCGAAAAACAAAAAGAATTCGGAAACCCACACGTATC |
| | | AGGAGGACTAAAAGAAGGAATCGAAACACTACTAATGACACAACG |
| | | ACCAGCACTATCAGGAGACGCAGTACAAAAAATGCTAGGACACTG |
| | | CACATTCGAACCAGCAGAACCAAAAGCAGCAAAAAACACATACAC |
| | | AGCAGAACGATTCATCTGACTAACAAAACTAAACAACCTACGAATC |
| | | CTAGAACAAGGATCAGAACGACCACTAACAGACACAGAACGAGCA |
| | | ACACTAATGGACGAACCATACCGAAAATCAAAACTAACATACGCA |
| | | CAAGCACGAAAACTACTATCACTAGAAGACACAGCATTCTTCAAAG |
| | | GACTACGATACGGAAAAGACAACGCAGAAGCATCAACACTAATGG |
| | | AAATGAAAGCATACCACACAATCTCACGAGCACTAGAAAAAGAAG |
| | | GACTAAAAGACAAAAAATCACCACTAAACCTATCACCAGAACTAC |
| | | AAGACGAAATCGGAACAGCATTCTCACTATTCAAAACAGACGAAG |
| | | ACATCACAGGACGACTAAAAGACCGAATCCAACCAGAAATCCTAG |
| | | AAGCACTACTAAAACACATCTCATTCGACAAATTCGTACAAATCTC |
| | | ACTAAAAGCACTACGACGAATCGTACCACTAATGGAACAAGGAAA |
| | | ACGATACGACGAAGCATGCGCAGAAATCTACGGAGACCACTACGG |
| | | AAAAAAAAACACAGAAGAAAAAATCTACCTACCACCAATCCCAGC |
| | | AGACGAAATCCGAAACCCAGTAGTACTACGAGCACTATCACAAGC |
| | | ACGAAAAGTAATCAACGGAGTAGTACGACGATACGGATCACCAGC |
| | | ACGAATCCACATCGAAACAGCACGAGAAGTAGGAAAATCATTCAA |
| | | AGACCGAAAAGAAATCGAAAAACGACAAGAAGAAAACCGAAAAG |
| | | ACCGAGAAAAAGCAGCAGCAAAATTCCGAGAATACTTCCCAAACTT |
| | | CGTAGGAGAACCAAAATCAAAAGACATCCTAAAACTACGACTATA |
| | | CGAACAACAACACGGAAAATGCCTATACTCAGGAAAAGAAATCAA |
| | | CCTAGGACGACTAAACGAAAAAGGATACGTAGAAATCGACGCAGC |
| | | ACTACCATTCTCACGAACATGAGACGACTCATTCAACAACAAAGTA |
| | | CTAGTACTAGGATCAGAAAACCAAAACAAAGGAAACCAAACACCA |
| | | TACGAATACTTCAACGGAAAAGACAACTCACGAGAATGACAAGAA |
| | | TTCAAAGCACGAGTAGAAACATCACGATTCCCACGATCAAAAAAAC |
| | | AACGAATCCTACTACAAAAATTCGACGAAGACGGATTCAAAGAAC |
| | | GAAACCTAAACGACACACGATACGTAAACCGATTCCTATGCCAATT |
| | | CGTAGCAGACCGAATGCGACTAACAGGAAAAGGAAAAAAACGAGT |
| | | ATTCGCATCAAACGGACAAATCACAAACCTACTACGAGGATTCTGA |
| | | GGACTACGAAAAGTACGAGCAGAAAACGACCGACACCACGCACTA |
| | | GACGCAGTAGTAGTAGCATGCTCAACAGTAGCAATGCAACAAAAA |
| | | ATCACACGATTCGTACGATACAAAGAAATGAACGCATTCGACGGAA |
| | | AAACAATCGACAAAGAAACAGGAGAAGTACTACACCAAAAAACAC |
| | | ACTTCCCACAACCATGAGAATTCTTCGCACAAGAAGTAATGATCCG |
| | | AGTATTCGGAAAACCAGACGGAAAACCAGAATTCGAAGAAGCAGA |
| | | CACACCAGAAAAACTACGAACACTACTAGCAGAAAAACTATCATC |
| | | ACGACCAGAAGCAGTACACGAATACGTAACACCACTATTCGTATCA |
| | | CGAGCACCAAACCGAAAAATGTCAGGACAAGGACACATGGAAACA |
| | | GTAAAATCAGCAAAACGACTAGACGAAGGAGTATCAGTACTACGA |
| | | GTACCACTAACACAACTAAAACTAAAAGACCTAGAAAAAATGGTA |
| | | AACCGAGAACGAGAACCAAAACTATACGAAGCACTAAAAGCACGA |
| | | CTAGAAGCACACAAAGACGACCCAGCAAAAGCATTCGCAGAACCA |
| | | TTCTACAAATACGACAAAGCAGGAAACCGAACACAACAAGTAAAA |
| | | GCAGTACGAGTAGAACAAGTACAAAAAACAGGAGTATGAGTACGA |
| | | AACCACAACGGAATCGCAGACAACGCAACAATGGTACGAGTAGAC |
| | | GTATTCGAAAAAGGAGACAAATACTACCTAGTACCAATCTACTCAT |
| | | GACAAGTAGCAAAAGGAATCCTACCAGACCGAGCAGTAGTAGCAT |
| | | ACGCAGACGAAGAAGACTGAACAGTAATCGACGAATCATTCCGATT |
| | | CAAATTCGTACTATACTCAAACGACCTAATCAAAGTACAACTAAAA |
| | | AAAGACTCATTCCTAGGATACTTCTCAGGACTAGACCGAGCAACAG |
| | | GAGCAATCTCACTACGAGAACACGACCTAGAAAAATCAAAAGGAA |
| | | AAGACGGAATGCACCGAATCGGAGTAAAAACAGCACTATCATTCC |
| | | AAAAATACCAAATCGACGAAATGGGAAAAGAAATCCGACCATGCC |
| | | GACTAAAAAAACGACCACCAGTACGA |
| 223 | Exemplary open | ATGGCTGCTTTTAAGCCTAATCCTATTAATTATATTCTTGGTCTTGAT |
| | reading frame for | ATTGGTATTGCTTCTGTTGGTTGGGCTATGGTTGAGATTGATGAGGA |
| | Nme3Cas9 HNH | GGAGAATCCTATTCGTCTTATTGATCTTGGTGTTCGTGTTTTTGAGC |
| | nickase | GTGCTGAGGTTCCTAAGACTGGTGATTCTCTTGCTATGGCTCGTCGT |
| | | CTTGCTCGTTCTGTTCGTCGTCTTACTCGTCGTCGTGCTCATCGTCTT |
| | | CTTCGTGCTCGTCGTCTTCTTAAGCGTGAGGGTGTTCTTCAGGCTGC |
| | | TGATTTTGATGAGAATGGTCTTATTAAGTCTCTTCCTAATACTCCTT |
| | | GGCAGCTTCGTGCTGCTGCTCTTGATCGTAAGCTTACTCCTCTTGAG |
| | | TGGTCTGCTGTTCTTCTTCATCTTATTAAGCATCGTGGTTATCTTTCT |
| | | CAGCGTAAGAATGAGGGTGAGACTGCTGATAAGGAGCTTGGTGCTC |
| | | TTCTTAAGGGTGTTGCTGATAATGCTCATGCTCTTCAGACTGGTGAT |
| | | TTTCGTACTCCTGCTGAGCTTGCTCTTAATAAGTTTGAGAAGGAGTG |
| | | TGGTCATATTCGTAATCAGCGTGGTGATTATTCTCATACTTTTTCTC |
| | | GTAAGGATCTTCAGGCTGAGCTTAATCTTCTTTTTGAGAAGCAGAA |
| | | GGAGTTTGGTAATCCTCATGTTTCTGGTGGTCTTAAGGAGGGTATTG |
| | | AGACTCTTCTTATGACTCAGCGTCCTGCTCTTTCTGGTGATGCTGTT |
| | | CAGAAGATGCTTGGTCATTGTACTTTTGAGCCTGCTGAGCCTAAGG |
| | | CTGCTAAGAATACTTATACTGCTGAGCGTTTTATTTGGCTTACTAAG |
| | | CTTAATAATCTTCGTATTCTTGAGCAGGGTTCTGAGCGTCCTCTTAC |
| | | TGATACTGAGCGTGCTACTCTTATGGATGAGCCTTATCGTAAGTCTA |
| | | AGCTTACTTATGCTCAGGCTCGTAAGCTTCTTTCTCTTGAGGATACT |
| | | GCTTTTTTTAAGGGTCTTCGTTATGGTAAGGATAATGCTGAGGCTTC |
| | | TACTCTTATGGAGATGAAGGCTTATCATACTATTTCTCGTGCTCTTG |
| | | AGAAGGAGGGTCTTAAGGATAAGAAGTCTCCTCTTAATCTTTCTCCT |
| | | GAGCTTCAGGATGAGATTGGTACTGCTTTTTCTCTTTTTAAGACTGA |
| | | TGAGGATATTACTGGTCGTCTTAAGGATCGTATTCAGCCTGAGATTC |
| | | TTGAGGCTCTTCTTAAGCATATTTCTTTTGATAAGTTTGTTCAGATTT |
| | | CTCTTAAGGCTCTTCGTCGTATTGTTCCTCTTATGGAGCAGGGTAAG |
| | | CGTTATGATGAGGCTTGTGCTGAGATTTATGGTGATCATTATGGTAA |
| | | GAAGAATACTGAGGAGAAGATTTATCTTCCTCCTATTCCTGCTGATG |
| | | AGATTCGTAATCCTGTTGTTCTTCGTGCTCTTTCTCAGGCTCGTAAG |
| | | GTTATTAATGGTGTTGTTCGTCGTTATGGTTCTCCTGCTCGTATTCAT |
| | | ATTGAGACTGCTCGTGAGGTTGGTAAGTCTTTTAAGGATCGTAAGG |
| | | AGATTGAGAAGCGTCAGGAGGAGAATCGTAAGGATCGTGAGAAGG |
| | | CTGCTGCTAAGTTTCGTGAGTATTTTCCTAATTTTGTTGGTGAGCCT |
| | | AAGTCTAAGGATATTCTTAAGCTTCGTCTTTATGAGCAGCAGCATG |
| | | GTAAGTGTCTTTATTCTGGTAAGGAGATTAATCTTGGTCGTCTTAAT |
| | | GAGAAGGGTTATGTTGAGATTGATGCTGCTCTTCCTTTTTCTCGTAC |
| | | TTGGGATGATTCTTTTAATAATAAGGTTCTTGTTCTTGGTTCTGAGA |
| | | ATCAGAATAAGGGTAATCAGACTCCTTATGAGTATTTTAATGGTAA |
| | | GGATAATTCTCGTGAGTGGCAGGAGTTTAAGGCTCGTGTTGAGACT |
| | | TCTCGTTTTCCTCGTTCTAAGAAGCAGCGTATTCTTCTTCAGAAGTT |
| | | TGATGAGGATGGTTTTAAGGAGCGTAATCTTAATGATACTCGTTAT |
| | | GTTAATCGTTTTCTTTGTCAGTTTGTTGCTGATCGTATGCGTCTTACT |
| | | GGTAAGGGTAAGAAGCGTGTTTTTGCTTCTAATGGTCAGATTACTA |
| | | ATCTTCTTCGTGGTTTTTGGGGTCTTCGTAAGGTTCGTGCTGAGAAT |
| | | GATCGTCATCATGCTCTTGATGCTGTTGTTGTTGCTTGTTCTACTGTT |
| | | GCTATGCAGCAGAAGATTACTCGTTTTGTTCGTTATAAGGAGATGA |
| | | ATGCTTTTGATGGTAAGACTATTGATAAGGAGACTGGTGAGGTTCT |
| | | TCATCAGAAGACTCATTTTCCTCAGCCTTGGGAGTTTTTTGCTCAGG |
| | | AGGTTATGATTCGTGTTTTTGGTAAGCCTGATGGTAAGCCTGAGTTT |
| | | GAGGAGGCTGATACTCCTGAGAAGCTTCGTACTCTTCTTGCTGAGA |
| | | AGCTTTCTTCTCGTCCTGAGGCTGTTCATGAGTATGTTACTCCTCTTT |
| | | TTGTTTCTCGTGCTCCTAATCGTAAGATGTCTGGTCAGGGTCATATG |
| | | GAGACTGTTAAGTCTGCTAAGCGTCTTGATGAGGGTGTTTCTGTTCT |
| | | TCGTGTTCCTCTTACTCAGCTTAAGCTTAAGGATCTTGAGAAGATGG |
| | | TTAATCGTGAGCGTGAGCCTAAGCTTTATGAGGCTCTTAAGGCTCGT |
| | | CTTGAGGCTCATAAGGATGATCCTGCTAAGGCTTTTGCTGAGCCTTT |
| | | TTATAAGTATGATAAGGCTGGTAATCGTACTCAGCAGGTTAAGGCT |
| | | GTTCGTGTTGAGCAGGTTCAGAAGACTGGTGTTTGGGTTCGTAATC |
| | | ATAATGGTATTGCTGATAATGCTACTATGGTTCGTGTTGATGTTTTT |
| | | GAGAAGGGTGATAAGTATTATCTTGTTCCTATTTATTCTTGGCAGGT |
| | | TGCTAAGGGTATTCTTCCTGATCGTGCTGTTGTTGCTTATGCTGATG |
| | | AGGAGGATTGGACTGTTATTGATGAGTCTTTTCGTTTTAAGTTTGTT |
| | | CTTTATTCTAATGATCTTATTAAGGTTCAGCTTAAGAAGGATTCTTT |
| | | TCTTGGTTATTTTTCTGGTCTTGATCGTGCTACTGGTGCTATTTCTCT |
| | | TCGTGAGCATGATCTTGAGAAGTCTAAGGGTAAGGATGGTATGCAT |
| | | CGTATTGGTGTTAAGACTGCTCTTTCTTTTCAGAAGTATCAGATTGA |
| | | TGAGATGGGTAAGGAGATTCGTCCTTGTCGTCTTAAGAAGCGTCCT |
| | | CCTGTTCGTUGA |
| 224 | Exemplary open | ATGGCCGCCTTCAAGCCCAACCCCATCAACTACATCCTGGGCCTGG |
| | reading frame for | ACATCGGCATCGCCTCCGTGGGCTGGGCCATGGTGGAGATCGACGA |
| | Nme3Cas9 HNH | GGAGGAGAACCCCATCCGGCTGATCGACCTGGGCGTGCGGGTGTTC |
| | nickase | GAGCGGGCCGAGGTGCCCAAGACCGGCGACTCCCTGGCCATGGCCC |
| | | GGCGGCTGGCCCGGTCCGTGCGGCGGCTGACCCGGCGGCGGGCCCA |
| | | CCGGCTGCTGCGGGCCCGGCGGCTGCTGAAGCGGGAGGGCGTGCTG |
| | | CAGGCCGCCGACTTCGACGAGAACGGCCTGATCAAGTCCCTGCCCA |
| | | ACACCCCCTGGCAGCTGCGGGCCGCCGCCCTGGACCGGAAGCTGAC |
| | | CCCCCTGGAGTGGTCCGCCGTGCTGCTGCACCTGATCAAGCACCGG |
| | | GGCTACCTGTCCCAGCGGAAGAACGAGGGCGAGACCGCCGACAAG |
| | | GAGCTGGGCGCCCTGCTGAAGGGCGTGGCCGACAACGCCCACGCCC |
| | | TGCAGACCGGCGACTTCCGGACCCCCGCCGAGCTGGCCCTGAACAA |
| | | GTTCGAGAAGGAGTGCGGCCACATCCGGAACCAGCGGGGCGACTA |
| | | CTCCCACACCTTCTCCCGGAAGGACCTGCAGGCCGAGCTGAACCTG |
| | | CTGTTCGAGAAGCAGAAGGAGTTCGGCAACCCCCACGTGTCCGGCG |
| | | GCCTGAAGGAGGGCATCGAGACCCTGCTGATGACCCAGCGGCCCGC |
| | | CCTGTCCGGCGACGCCGTGCAGAAGATGCTGGGCCACTGCACCTTC |
| | | GAGCCCGCCGAGCCCAAGGCCGCCAAGAACACCTACACCGCCGAG |
| | | CGGTTCATCTGGCTGACCAAGCTGAACAACCTGCGGATCCTGGAGC |
| | | AGGGCTCCGAGCGGCCCCTGACCGACACCGAGCGGGCCACCCTGAT |
| | | GGACGAGCCCTACCGGAAGTCCAAGCTGACCTACGCCCAGGCCCGG |
| | | AAGCTGCTGTCCCTGGAGGACACCGCCTTCTTCAAGGGCCTGCGGT |
| | | ACGGCAAGGACAACGCCGAGGCCTCCACCCTGATGGAGATGAAGG |
| | | CCTACCACACCATCTCCCGGGCCCTGGAGAAGGAGGGCCTGAAGGA |
| | | CAAGAAGTCCCCCCTGAACCTGTCCCCCGAGCTGCAGGACGAGATC |
| | | GGCACCGCCTTCTCCCTGTTCAAGACCGACGAGGACATCACCGGCC |
| | | GGCTGAAGGACCGGATCCAGCCCGAGATCCTGGAGGCCCTGCTGAA |
| | | GCACATCTCCTTCGACAAGTTCGTGCAGATCTCCCTGAAGGCCCTGC |
| | | GGCGGATCGTGCCCCTGATGGAGCAGGGCAAGCGGTACGACGAGG |
| | | CCTGCGCCGAGATCTACGGCGACCACTACGGCAAGAAGAACACCG |
| | | AGGAGAAGATCTACCTGCCCCCCATCCCCGCCGACGAGATCCGGAA |
| | | CCCCGTGGTGCTGCGGGCCCTGTCCCAGGCCCGGAAGGTGATCAAC |
| | | GGCGTGGTGCGGCGGTACGGCTCCCCCGCCCGGATCCACATCGAGA |
| | | CCGCCCGGGAGGTGGGCAAGTCCTTCAAGGACCGGAAGGAGATCG |
| | | AGAAGCGGCAGGAGGAGAACCGGAAGGACCGGGAGAAGGCCGCC |
| | | GCCAAGTTCCGGGAGTACTTCCCCAACTTCGTGGGCGAGCCCAAGT |
| | | CCAAGGACATCCTGAAGCTGCGGCTGTACGAGCAGCAGCACGGCA |
| | | AGTGCCTGTACTCCGGCAAGGAGATCAACCTGGGCCGGCTGAACGA |
| | | GAAGGGCTACGTGGAGATCGACGCCGCCCTGCCCTTCTCCCGGACC |
| | | TGGGACGACTCCTTCAACAACAAGGTGCTGGTGCTGGGCTCCGAGA |
| | | ACCAGAACAAGGGCAACCAGACCCCCTACGAGTACTTCAACGGCA |
| | | AGGACAACTCCCGGGAGTGGCAGGAGTTCAAGGCCCGGGTGGAGA |
| | | CCTCCCGGTTCCCCCGGTCCAAGAAGCAGCGGATCCTGCTGCAGAA |
| | | GTTCGACGAGGACGGCTTCAAGGAGCGGAACCTGAACGACACCCG |
| | | GTACGTGAACCGGTTCCTGTGCCAGTTCGTGGCCGACCGGATGCGG |
| | | CTGACCGGCAAGGGCAAGAAGCGGGTGTTCGCCTCCAACGGCCAG |
| | | ATCACCAACCTGCTGCGGGGCTTCTGGGGCCTGCGGAAGGTGCGGG |
| | | CCGAGAACGACCGGCACCACGCCCTGGACGCCGTGGTGGTGGCCTG |
| | | CTCCACCGTGGCCATGCAGCAGAAGATCACCCGGTTCGTGCGGTAC |
| | | AAGGAGATGAACGCCTTCGACGGCAAGACCATCGACAAGGAGACC |
| | | GGCGAGGTGCTGCACCAGAAGACCCACTTCCCCCAGCCCTGGGAGT |
| | | TCTTCGCCCAGGAGGTGATGATCCGGGTGTTCGGCAAGCCCGACGG |
| | | CAAGCCCGAGTTCGAGGAGGCCGACACCCCCGAGAAGCTGCGGAC |
| | | CCTGCTGGCCGAGAAGCTGTCCTCCCGGCCCGAGGCCGTGCACGAG |
| | | TACGTGACCCCCCTGTTCGTGTCCCGGGCCCCCAACCGGAAGATGT |
| | | CCGGCCAGGGCCACATGGAGACCGTGAAGTCCGCCAAGCGGCTGG |
| | | ACGAGGGCGTGTCCGTGCTGCGGGTGCCCCTGACCCAGCTGAAGCT |
| | | GAAGGACCTGGAGAAGATGGTGAACCGGGAGCGGGAGCCCAAGCT |
| | | GTACGAGGCCCTGAAGGCCCGGCTGGAGGCCCACAAGGACGACCC |
| | | CGCCAAGGCCTTCGCCGAGCCCTTCTACAAGTACGACAAGGCCGGC |
| | | AACCGGACCCAGCAGGTGAAGGCCGTGCGGGTGGAGCAGGTGCAG |
| | | AAGACCGGCGTGTGGGTGCGGAACCACAACGGCATCGCCGACAAC |
| | | GCCACCATGGTGCGGGTGGACGTGTTCGAGAAGGGCGACAAGTACT |
| | | ACCTGGTGCCCATCTACTCCTGGCAGGTGGCCAAGGGCATCCTGCC |
| | | CGACCGGGCCGTGGTGGCCTACGCCGACGAGGAGGACTGGACCGT |
| | | GATCGACGAGTCCTTCCGGTTCAAGTTCGTGCTGTACTCCAACGACC |
| | | TGATCAAGGTGCAGCTGAAGAAGGACTCCTTCCTGGGCTACTTCTC |
| | | CGGCCTGGACCGGGCCACCGGCGCCATCTCCCTGCGGGAGCACGAC |
| | | CTGGAGAAGTCCAAGGGCAAGGACGGCATGCACCGGATCGGCGTG |
| | | AAGACCGCCCTGTCCTTCCAGAAGTACCAGATCGACGAGATGGGCA |
| | | AGGAGATCCGGCCCTGCCGGCTGAAGAAGCGGCCCCCCGTGCGGU |
| | | GA |
| 225 | Exemplary open | ATGGCAGCATTCAAACCAAACCCAATCAACTACATCCTAGGACTAG |
| | reading frame for | ACATCGGAATCGCATCAGTAGGATGAGCAATGGTAGAAATCGACG |
| | Nme3Cas9 HNH | AAGAAGAAAACCCAATCCGACTAATCGACCTAGGAGTACGAGTATT |
| | nickase | CGAACGAGCAGAAGTACCAAAAACAGGAGACTCACTAGCAATGGC |
| | | ACGACGACTAGCACGATCAGTACGACGACTAACACGACGACGAGC |
| | | ACACCGACTACTACGAGCACGACGACTACTAAAACGAGAAGGAGT |
| | | ACTACAAGCAGCAGACTTCGACGAAAACGGACTAATCAAATCACTA |
| | | CCAAACACACCATGACAACTACGAGCAGCAGCACTAGACCGAAAA |
| | | CTAACACCACTAGAATGATCAGCAGTACTACTACACCTAATCAAAC |
| | | ACCGAGGATACCTATCACAACGAAAAAACGAAGGAGAAACAGCAG |
| | | ACAAAGAACTAGGAGCACTACTAAAAGGAGTAGCAGACAACGCAC |
| | | ACGCACTACAAACAGGAGACTTCCGAACACCAGCAGAACTAGCAC |
| | | TAAACAAATTCGAAAAAGAATGCGGACACATCCGAAACCAACGAG |
| | | GAGACTACTCACACACATTCTCACGAAAAGACCTACAAGCAGAACT |
| | | AAACCTACTATTCGAAAAACAAAAAGAATTCGGAAACCCACACGT |
| | | ATCAGGAGGACTAAAAGAAGGAATCGAAACACTACTAATGACACA |
| | | ACGACCAGCACTATCAGGAGACGCAGTACAAAAAATGCTAGGACA |
| | | CTGCACATTCGAACCAGCAGAACCAAAAGCAGCAAAAAACACATA |
| | | CACAGCAGAACGATTCATCTGACTAACAAAACTAAACAACCTACGA |
| | | ATCCTAGAACAAGGATCAGAACGACCACTAACAGACACAGAACGA |
| | | GCAACACTAATGGACGAACCATACCGAAAATCAAAACTAACATAC |
| | | GCACAAGCACGAAAACTACTATCACTAGAAGACACAGCATTCTTCA |
| | | AAGGACTACGATACGGAAAAGACAACGCAGAAGCATCAACACTAA |
| | | TGGAAATGAAAGCATACCACACAATCTCACGAGCACTAGAAAAAG |
| | | AAGGACTAAAAGACAAAAAATCACCACTAAACCTATCACCAGAAC |
| | | TACAAGACGAAATCGGAACAGCATTCTCACTATTCAAAACAGACGA |
| | | AGACATCACAGGACGACTAAAAGACCGAATCCAACCAGAAATCCT |
| | | AGAAGCACTACTAAAACACATCTCATTCGACAAATTCGTACAAATC |
| | | TCACTAAAAGCACTACGACGAATCGTACCACTAATGGAACAAGGA |
| | | AAACGATACGACGAAGCATGCGCAGAAATCTACGGAGACCACTAC |
| | | GGAAAAAAAAACACAGAAGAAAAAATCTACCTACCACCAATCCCA |
| | | GCAGACGAAATCCGAAACCCAGTAGTACTACGAGCACTATCACAA |
| | | GCACGAAAAGTAATCAACGGAGTAGTACGACGATACGGATCACCA |
| | | GCACGAATCCACATCGAAACAGCACGAGAAGTAGGAAAATCATTC |
| | | AAAGACCGAAAAGAAATCGAAAAACGACAAGAAGAAAACCGAAA |
| | | AGACCGAGAAAAAGCAGCAGCAAAATTCCGAGAATACTTCCCAAA |
| | | CTTCGTAGGAGAACCAAAATCAAAAGACATCCTAAAACTACGACTA |
| | | TACGAACAACAACACGGAAAATGCCTATACTCAGGAAAAGAAATC |
| | | AACCTAGGACGACTAAACGAAAAAGGATACGTAGAAATCGACGCA |
| | | GCACTACCATTCTCACGAACATGAGACGACTCATTCAACAACAAAG |
| | | TACTAGTACTAGGATCAGAAAACCAAAACAAAGGAAACCAAACAC |
| | | CATACGAATACTTCAACGGAAAAGACAACTCACGAGAATGACAAG |
| | | AATTCAAAGCACGAGTAGAAACATCACGATTCCCACGATCAAAAA |
| | | AACAACGAATCCTACTACAAAAATTCGACGAAGACGGATTCAAAG |
| | | AACGAAACCTAAACGACACACGATACGTAAACCGATTCCTATGCCA |
| | | ATTCGTAGCAGACCGAATGCGACTAACAGGAAAAGGAAAAAAACG |
| | | AGTATTCGCATCAAACGGACAAATCACAAACCTACTACGAGGATTC |
| | | TGAGGACTACGAAAAGTACGAGCAGAAAACGACCGACACCACGCA |
| | | CTAGACGCAGTAGTAGTAGCATGCTCAACAGTAGCAATGCAACAAA |
| | | AAATCACACGATTCGTACGATACAAAGAAATGAACGCATTCGACGG |
| | | AAAAACAATCGACAAAGAAACAGGAGAAGTACTACACCAAAAAAC |
| | | ACACTTCCCACAACCATGAGAATTCTTCGCACAAGAAGTAATGATC |
| | | CGAGTATTCGGAAAACCAGACGGAAAACCAGAATTCGAAGAAGCA |
| | | GACACACCAGAAAAACTACGAACACTACTAGCAGAAAAACTATCA |
| | | TCACGACCAGAAGCAGTACACGAATACGTAACACCACTATTCGTAT |
| | | CACGAGCACCAAACCGAAAAATGTCAGGACAAGGACACATGGAAA |
| | | CAGTAAAATCAGCAAAACGACTAGACGAAGGAGTATCAGTACTAC |
| | | GAGTACCACTAACACAACTAAAACTAAAAGACCTAGAAAAAATGG |
| | | TAAACCGAGAACGAGAACCAAAACTATACGAAGCACTAAAAGCAC |
| | | GACTAGAAGCACACAAAGACGACCCAGCAAAAGCATTCGCAGAAC |
| | | CATTCTACAAATACGACAAAGCAGGAAACCGAACACAACAAGTAA |
| | | AAGCAGTACGAGTAGAACAAGTACAAAAAACAGGAGTATGAGTAC |
| | | GAAACCACAACGGAATCGCAGACAACGCAACAATGGTACGAGTAG |
| | | ACGTATTCGAAAAAGGAGACAAATACTACCTAGTACCAATCTACTC |
| | | ATGACAAGTAGCAAAAGGAATCCTACCAGACCGAGCAGTAGTAGC |
| | | ATACGCAGACGAAGAAGACTGAACAGTAATCGACGAATCATTCCG |
| | | ATTCAAATTCGTACTATACTCAAACGACCTAATCAAAGTACAACTA |
| | | AAAAAAGACTCATTCCTAGGATACTTCTCAGGACTAGACCGAGCAA |
| | | CAGGAGCAATCTCACTACGAGAACACGACCTAGAAAAATCAAAAG |
| | | GAAAAGACGGAATGCACCGAATCGGAGTAAAAACAGCACTATCAT |
| | | TCCAAAAATACCAAATCGACGAAATGGGAAAAGAAATCCGACCAT |
| | | GCCGACTAAAAAAACGACCACCAGTACGAUAA |
| 226 | Exemplary | GUUUUAGAGCUAGAAAUAGCAAGUUAAAAUAAGGCUAGUCCGUU |
| | SpyCas9 sgRNA- | AUCAACUUGAAAAAGUGGCACCGAGUCGGUGC |
| | 1 | |
| 227 | Exemplary | GUUUUAGAGCUAGAAAUAGCAAGUUAAAAUAAGGCUAGUCCGUU |
| | nucleotide | AUCAACUUGAAAAAGUGGCACCGAGUCGGUGCUUUU |
| | sequence | |
| | following 3′ end | |
| | of guide sequence | |
| 228 | Exemplary | mN*mN*mN*NNNNNNNNNNNNNNNNNGUUUUAGAmGmCmUmAmG |
| | modified | mAmAmAmUmAmGmCAAGUUAAAAUAAGGCUAGUCCGUUAUCAm |
| | SpyCas9 motif | AmCmUmUmGmAmAmAmAmAmGmUmGmGmCmAmCmCmGmAmGm |
| | | UmCmGmGmUmGmCmU*mU*mU*mU |
| 229 | Exemplary | (mN*3N17GUUUUAGAmGmCmUmAmGmAmAmAmUmAmGmCAAGU |
| | modified | UAAAAUAAGGCUAGUCCGUUAUCAmAmCmUmUmGmAmA |
| | SpyCas9 | mAmAmAmGmUmGmGmCmAmCmCmGmAmGmUmCmGmGmUmGmC |
| | conserved portion | |
| | motif | |
| 230 | Exemplary | (mN*3N17GUUUUAGAmGmCmUmAmGmAmAmAmUmAmGmCAAGU |
| | modified | UAAAAUAAGGCUAGUCCGUUAUCAmAmCmUmUmGmAmA |
| | SpyCas9 | mAmAmAmGmUmGmGmCmAmCmCmGmAmGmUmCmGmGmUmGmC |
| | conserved portion | mU |
| | motif | |
| 231 | Exemplary | (mN*3N17GUUUUAGAmGmCmUmAmGmAmAmAmUmAmGmCAAGU |
| | modified | UAAAAUAAGGCUAGUCCGUUAUCAACUUGGCACCGAGUCGG*mU* |
| | SpyCas9 | mG*mC |
| | conserved portion | |
| | motif | |
| 232 | Exemplary | (mN*3N17GUUUUAGAmGmCmUmAmGmAmAmAmUmAmGmCAAGU |
| | modified | UAAAAUAAGGCUAGUCCGUUAUCAAAAUGGCACCGAGUC |
| | SpyCas9 | GG*mU*mG*mC |
| | conserved portion | |
| | motif | |
| 233 | Exemplary | (mN*3N17GUUUUAGAmGmCmUmAmGmAmAmAmUmAmGmCAAGU |
| | modified | UAAAAUAAGGCUAGUCCGUUAUCAmAmAmAmUmGmGmC |
| | SpyCas9 | mAmCmCmGmAmGmUmCmGmG*mU*mG*mC |
| | conserved portion | |
| | motif | |
| 234 | Exemplary | (mN*3N17GUUUUAGAmGmCmUmAmGmAmAmAmUmAmGmCAAGU |
| | modified | UAAAAUAAGGCUAGUCCGUUAUCACGAAAGGGCACCGAGUCGG* |
| | SpyCas9 | mU*mG*mC |
| | conserved portion | |
| | motif | |
| 235 | Exemplary | (mN*3N17GUUUUAGAmGmCmUmAmGmAmAmAmUmAmGmCAAGU |
| | modified | UAAAAUAAGGCUAGUCCGUUAUCAmCmGmAmAmAmGmG |
| | SpyCas9 | mGmCmAmCmCmGmAmGmUmCmGmG*mU*mG*mC |
| | conserved portion | |
| | motif | |
| 236 | Exemplary | (mN*3N17GUUUUAGAmGmCmUmAmGmAmAmAmUmAmGmCAAGU |
| | modified | UAAAAUAAGGCUAGUCCGUUAUCACGAAAGGGCACCG |
| | SpyCas9 | AGUCGGU*mG*mC*mU |
| | conserved portion | |
| | motif | |
| 237 | Exemplary | (mN*3N17GUUUUAGAmGmCmUmAmGmAmAmAmUmAmGmCAAGU |
| | modified | UAAAAUAAGGCUAGUCCGUUAUCACGAAAGGGCACCGAGUCGGm |
| | SpyCas9 | UmGmC*mU |
| | conserved portion | |
| | motif | |
| 238 | Exemplary | (mN*3N17GUUUfUAGmAmGmCmUmAmGmAmAmAmUmAmGmCmAm |
| | modified | AGUfUmAfAmAfAmUAmAmGmGmCmUmAGUmCmCGUfUAmUmCAm |
| | SpyCas9 | CmGmAmAmAmGmGmGmCmAmCmCmGmAmGmUmCmGmGmU*mG* |
| | conserved portion | mC*mU |
| | motif | |
| 239 | Exemplary | (mN*3N17GUUUUAGAmGmCmUmAmGmAmAmAmUmAmGmCAAGU |
| | modified | UAAAAUAAGGCUAGUCCGUUAUCACGAAAGGGCACCGAGUCGGm |
| | SpyCas9 | U*mG*mC*mU |
| | conserved portion | |
| | motif | |
| 240 | Exemplary | (mN*3N17GUUUUAGAmGmCmUmAmGmAmAmAmUmAmGmCAAGU |
| | modified | UAAAAUAAGGCUAGUCCGUUAUCAmAmCmUmUmGmAmAmAmAm |
| | SpyCas9 | AmGmUmGmGmCmAmCmCmGmAmGmUmCmGmGmUmGmC |
| | conserved portion | |
| | motif | |
| 241 | Exemplary | (mN*3N17GUUUUAGAmGmCmUmAmGmAmAmAmUmAmGmCAAGU |
| | modified | UAAAAUAAGGCUAGUCCGUUAUCAmAmCmUmUmGmAmAmAmAm |
| | SpyCas9 | AmGmUmGmGmCmAmCmCmGmAmGmUmCmGmGmUmGmCmU |
| | conserved portion | |
| | motif | |
| 242 | Exemplary | (mN*3N17GUUUUAGAmGmCmUmAmGmAmAmAmUmAmGmCAAGU |
| | modified | UAAAAUAAGGCUAGUCCGUUAUCAACUUGGCACCGAGUCGG*mU* |
| | SpyCas9 | mG*mC |
| | conserved portion | |
| | motif | |
| 243 | Exemplary | GUUGUAGCUCCCUUUCUCAUUUCGGAAACGAAAUGAGAACCGUU |
| | NmeCas9 | GCUACAAUAAGGCCGUCUGAAAAGAUGUGCCGCAACGCUCUGCCC |
| | sgRNA-1 | CUUAAAGCUUCUGCUUUAAGGGGCAUCGUUUA |
| 244 | Exemplary | GUUGUAGCUCCCUGAAACCGUUGCUACAAUAAGGCCGUCGAAAG |
| | unmodified | AUGUGCCGCAACGCUCUGCCUUCUGGCAUCGUU |
| | conserved portion | |
| | nucleotide | |
| | sequence | |
| 245 | Exemplary | GUUGUAGCUCCCUGGAAACCCGUUGCUACAAUAAGGCCGUCGAA |
| | unmodified | AGAUGUGCCGCAACGCUCUGCCUUCUGGCAUCGUUUAUU |
| | conserved portion | |
| | nucleotide | |
| | sequence | |
| 246 | Exemplary | GUUGmUmAmGmCUCCCmUmGmAmAmAmCmCGUUmGmCUAmCAA |
| | modified | U*AAGmGmCCmGmUmCmGmAmAmAmGmAmUGUGCmCGCmAmAm |
| | conserved portion | CmGCUCUmGmCCmUmUmCmUGmGCmAmUC*mG*mU*mU |
| | motif | |
| 247 | Exemplary | GUUGmUmAmGmCUCCCmUmGmAmAmAmCmCGUUmGmCUAmCAA |
| | modified | U*AAGmGmCCmGmUmCmGmAmAmAmGmAmUGUGCmCGmCAAmC |
| | conserved portion | GCUCUmGmCCmUmUmCmUGGCAUCG*mU*mU |
| | motif | |
| 248 | Exemplary | mN*mNNNNNNNNmNNNmNNNNNNNNNNNN |
| | modified | mGUUGmUmAmGmCUCCCmUmGmAmAmAmCmCGUUmGmCUAmCA |
| | conserved portion | AU*AAGmGmCCmGmUmCmGmAmAmAmGmAmUGUGCmCGCmAmA |
| | motif | mCmGCUCUmGmCCmUmUmCmUGmGCmAmUC*mG*mU*mU |
| 249 | Exemplary | (N)20- |
| | modified | 25GUUGmUmAmGmCUCCCmUmGmAmAmAmCmCGUUmGmCUAmCA |
| | conserved portion | AU*AAGmGmCCmGmUmCmGm |
| | motif | AmAmAmGmAmUGUGCmCGCmAmAmCmGCUCUmGmCCmUmUmCm |
| | | UGmGCmAmUC*mG*mU*mU |
| 250 | Exemplary | mN*mN*mN*mNmNNNmNmNNmNNmNNNNNmNNNNmNNNmGUUG |
| | modified | mUmAmGmCUCCCmUmGmAmAmAmCmCGUUmGmCUAmCAAU*AA |
| | conserved portion | GmGmCCmGmUmCmGmAmAmAmGmAmUGUGCmCGmCAAmCGCUC |
| | motif | UmGmCCmUmUmCmUGGCAUCG*mU*mU |
| 251 | G000562 | CCAAUAUCAGGAGACUAGGA |
| 252 | G013515 | CCAUCGUAAGCAAACCUUAG |
| 253 | G013519 | GCAAGGAGAGAGAUGGCUCC |
| 254 | G013520 | GAGAGAUGGCUCCAGGAAAU |
| 255 | G013523 | GGUGACACACCCCCAUUUCC |
| 256 | G013533 | AGACCCAAUAUCAGGAGACU |
| 257 | G013543 | UGUCCCUAGUGGCCCCACUG |
| 258 | G013559 | CCGGCCCUGGGAAUAUAAGG |
| 259 | G013562 | AAUAUAAGGUGGUCCCAGCU |
| 260 | G013563 | AUAUAAGGUGGUCCCAGCUC |
| 261 | G013564 | UAUAAGGUGGUCCCAGCUCG |
| 262 | G013565 | GGAUCCUGUGUCCCCGAGCU |
| 263 | G013582 | CCUGUCAUGGCAUCUUCCAG |
| 264 | G013584 | CCCUGGAAGAUGCCAUGACA |
| 265 | G000562 | CCAAUAUCAGGAGACUAGGAGUUUUAGAGCUAGAAAUAGCAAGU |
| | (exemplary full | UAAAAUAAGGCUAGUCCGUUAUCAACUUGAAAAAGUGGCACCGA |
| | sequence) | GUCGGUGCUUUU |
| 266 | G013515 | CCAUCGUAAGCAAACCUUAGGUUUUAGAGCUAGAAAUAGCAAGU |
| | (exemplary full | UAAAAUAAGGCUAGUCCGUUAUCAACUUGAAAAAGUGGCACCGA |
| | sequence) | GUCGGUGCUUUU |
| 267 | G013519 | GCAAGGAGAGAGAUGGCUCCGUUUUAGAGCUAGAAAUAGCAAGU |
| | (exemplary full | UAAAAUAAGGCUAGUCCGUUAUCAACUUGAAAAAGUGGCACCGA |
| | sequence) | GUCGGUGCUUUU |
| 268 | G013520 | GAGAGAUGGCUCCAGGAAAUGUUUUAGAGCUAGAAAUAGCAAGU |
| | (exemplary full | UAAAAUAAGGCUAGUCCGUUAUCAACUUGAAAAAGUGGCACCGA |
| | sequence) | GUCGGUGCUUUU |
| 269 | G013523 | GGUGACACACCCCCAUUUCCGUUUUAGAGCUAGAAAUAGCAAGU |
| | (exemplary full | UAAAAUAAGGCUAGUCCGUUAUCAACUUGAAAAAGUGGCACCGA |
| | sequence) | GUCGGUGCUUUU |
| 270 | G013533 | AGACCCAAUAUCAGGAGACUGUUUUAGAGCUAGAAAUAGCAAGU |
| | (exemplary full | UAAAAUAAGGCUAGUCCGUUAUCAACUUGAAAAAGUGGCACCGA |
| | sequence) | GUCGGUGCUUUU |
| 271 | G013543 | UGUCCCUAGUGGCCCCACUGGUUUUAGAGCUAGAAAUAGCAAGU |
| | (exemplary full | UAAAAUAAGGCUAGUCCGUUAUCAACUUGAAAAAGUGGCACCGA |
| | sequence) | GUCGGUGCUUUU |
| 272 | G013559 | CCGGCCCUGGGAAUAUAAGGGUUUUAGAGCUAGAAAUAGCAAGU |
| | (exemplary full | UAAAAUAAGGCUAGUCCGUUAUCAACUUGAAAAAGUGGCACCGA |
| | sequence) | GUCGGUGCUUUU |
| 273 | G013562 | AAUAUAAGGUGGUCCCAGCUGUUUUAGAGCUAGAAAUAGCAAGU |
| | (exemplary full | UAAAAUAAGGCUAGUCCGUUAUCAACUUGAAAAAGUGGCACCGA |
| | sequence) | GUCGGUGCUUUU |
| 274 | G013563 | AUAUAAGGUGGUCCCAGCUCGUUUUAGAGCUAGAAAUAGCAAGU |
| | (exemplary full | UAAAAUAAGGCUAGUCCGUUAUCAACUUGAAAAAGUGGCACCGA |
| | sequence) | GUCGGUGCUUUU |
| 275 | G013564 | UAUAAGGUGGUCCCAGCUCGGUUUUAGAGCUAGAAAUAGCAAGU |
| | (exemplary full | UAAAAUAAGGCUAGUCCGUUAUCAACUUGAAAAAGUGGCACCGA |
| | sequence) | GUCGGUGCUUUU |
| 276 | G013565 | GGAUCCUGUGUCCCCGAGCUGUUUUAGAGCUAGAAAUAGCAAGU |
| | (exemplary full | UAAAAUAAGGCUAGUCCGUUAUCAACUUGAAAAAGUGGCACCGA |
| | sequence) | GUCGGUGCUUUU |
| 277 | G013582 | CCUGUCAUGGCAUCUUCCAGGUUUUAGAGCUAGAAAUAGCAAGU |
| | (exemplary full | UAAAAUAAGGCUAGUCCGUUAUCAACUUGAAAAAGUGGCACCGA |
| | sequence) | GUCGGUGCUUUU |
| 278 | G013584 | CCCUGGAAGAUGCCAUGACAGUUUUAGAGCUAGAAAUAGCAAGU |
| | (exemplary full | UAAAAUAAGGCUAGUCCGUUAUCAACUUGAAAAAGUGGCACCGA |
| | sequence) | GUCGGUGCUUUU |
| 279 | G000562 | mC*mC*mA*AUAUCAGGAGACUAGGAGUUUUAGAmGmCmUmAmG |
| | (exemplary mod | mAmAmAmUmAmGmCAAGUUAAAAUAAGGCUAGUCCGUUAUCAm |
| | sequence) | AmCmUmUmGmAmAmAmAmAmGmUmGmGmCmAmCmCmGmAmGm |
| | | UmCmGmGmUmGmCmU*mU*mU*mU |
| 280 | G013515 | mC*mC*mA*UCGUAAGCAAACCUUAGGUUUUAGAmGmCmUmAmG |
| | (exemplary mod | mAmAmAmUmAmGmCAAGUUAAAAUAAGGCUAGUCCGUUAUCAm |
| | sequence) | AmCmUmUmGmAmAmAmAmAmGmUmGmGmCmAmCmCmGmAmGm |
| | | UmCmGmGmUmGmCmU*mU*mU*mU |
| 281 | G013519 | mG*mC*mA*AGGAGAGAGAUGGCUCCGUUUUAGAmGmCmUmAmG |
| | (exemplary mod | mAmAmAmUmAmGmCAAGUUAAAAUAAGGCUAGUCCGUUAUCAm |
| | sequence) | AmCmUmUmGmAmAmAmAmAmGmUmGmGmCmAmCmCmGmAmGm |
| | | UmCmGmGmUmGmCmU*mU*mU*mU |
| 282 | G013520 | mG*mA*mG*AGAUGGCUCCAGGAAAUGUUUUAGAmGmCmUmAmG |
| | (exemplary mod | mAmAmAmUmAmGmCAAGUUAAAAUAAGGCUAGUCCGUUAUCAm |
| | sequence) | AmCmUmUmGmAmAmAmAmAmGmUmGmGmCmAmCmCmGmAmGm |
| | | UmCmGmGmUmGmCmU*mU*mU*mU |
| 283 | G013523 | mG*mG*mU*GACACACCCCCAUUUCCGUUUUAGAmGmCmUmAmGm |
| | (exemplary mod | AmAmAmUmAmGmCAAGUUAAAAUAAGGCUAGUCCGUUAUCAmA |
| | sequence) | mCmUmUmGmAmAmAmAmAmGmUmGmGmCmAmCmCmGmAmGmU |
| | | mCmGmGmUmGmCmU*mU*mU*mU |
| 284 | G013533 | mA*mG*mA*CCCAAUAUCAGGAGACUGUUUUAGAmGmCmUmAmG |
| | (exemplary mod | mAmAmAmUmAmGmCAAGUUAAAAUAAGGCUAGUCCGUUAUCAm |
| | sequence) | AmCmUmUmGmAmAmAmAmAmGmUmGmGmCmAmCmCmGmAmGm |
| | | UmCmGmGmUmGmCmU*mU*mU*mU |
| 285 | G013543 | mU*mG*mU*CCCUAGUGGCCCCACUGGUUUUAGAmGmCmUmAmG |
| | (exemplary mod | mAmAmAmUmAmGmCAAGUUAAAAUAAGGCUAGUCCGUUAUCAm |
| | sequence) | AmCmUmUmGmAmAmAmAmAmGmUmGmGmCmAmCmCmGmAmGm |
| | | UmCmGmGmUmGmCmU*mU*mU*mU |
| 286 | G013559 | mC*mC*mG*GCCCUGGGAAUAUAAGGGUUUUAGAmGmCmUmAmG |
| | (exemplary mod | mAmAmAmUmAmGmCAAGUUAAAAUAAGGCUAGUCCGUUAUCAm |
| | sequence) | AmCmUmUmGmAmAmAmAmAmGmUmGmGmCmAmCmCmGmAmGm |
| | | UmCmGmGmUmGmCmU*mU*mU*mU |
| 287 | G013562 | mA*mA*mU*AUAAGGUGGUCCCAGCUGUUUUAGAmGmCmUmAmG |
| | (exemplary mod | mAmAmAmUmAmGmCAAGUUAAAAUAAGGCUAGUCCGUUAUCAm |
| | sequence) | AmCmUmUmGmAmAmAmAmAmGmUmGmGmCmAmCmCmGmAmGm |
| | | UmCmGmGmUmGmCmU*mU*mU*mU |
| 288 | G013563 | mA*mU*mA*UAAGGUGGUCCCAGCUCGUUUUAGAmGmCmUmAmG |
| | (exemplary mod | mAmAmAmUmAmGmCAAGUUAAAAUAAGGCUAGUCCGUUAUCAm |
| | sequence) | AmCmUmUmGmAmAmAmAmAmGmUmGmGmCmAmCmCmGmAmGm |
| | | UmCmGmGmUmGmCmU*mU*mU*mU |
| 289 | G013564 | mU*mA*mU*AAGGUGGUCCCAGCUCGGUUUUAGAmGmCmUmAmG |
| | (exemplary mod | mAmAmAmUmAmGmCAAGUUAAAAUAAGGCUAGUCCGUUAUCAm |
| | sequence) | AmCmUmUmGmAmAmAmAmAmGmUmGmGmCmAmCmCmGmAmGm |
| | | UmCmGmGmUmGmCmU*mU*mU*mU |
| 290 | G013565 | mG*mG*mA*UCCUGUGUCCCCGAGCUGUUUUAGAmGmCmUmAmG |
| | (exemplary mod | mAmAmAmUmAmGmCAAGUUAAAAUAAGGCUAGUCCGUUAUCAm |
| | sequence) | AmCmUmUmGmAmAmAmAmAmGmUmGmGmCmAmCmCmGmAmGm |
| | | UmCmGmGmUmGmCmU*mU*mU*mU |
| 291 | G013582 | mC*mC*mU*GUCAUGGCAUCUUCCAGGUUUUAGAmGmCmUmAmG |
| | (exemplary mod | mAmAmAmUmAmGmCAAGUUAAAAUAAGGCUAGUCCGUUAUCAm |
| | sequence) | AmCmUmUmGmAmAmAmAmAmGmUmGmGmCmAmCmCmGmAmGm |
| | | UmCmGmGmUmGmCmU*mU*mU*mU |
| 292 | G013584 | mC*mC*mC*UGGAAGAUGCCAUGACAGUUUUAGAmGmCmUmAmG |
| | (exemplary mod | mAmAmAmUmAmGmCAAGUUAAAAUAAGGCUAGUCCGUUAUCAm |
| | sequence) | AmCmUmUmGmAmAmAmAmAmGmUmGmGmCmAmCmCmGmAmGm |
| | | UmCmGmGmUmGmCmU*mU*mU*mU |
| 293 | Open reading | AUGGACAAGAAGUACAGCAUCGGACUGGACAUCGGAACAAACAG |
| | frame for Cas9 | CGUCGGAUGGGCAGUCAUCACAGACGAAUACAAGGUCCCGAGCA |
| | | AGAAGUUCAAGGUCCUGGGAAACACAGACAGACACAGCAUCAAG |
| | | AAGAACCUGAUCGGAGCACUGCUGUUCGACAGCGGAGAAACAGC |
| | | AGAAGCAACAAGACUGAAGAGAACAGCAAGAAGAAGAUACACAA |
| | | GAAGAAAGAACAGAAUCUGCUACCUGCAGGAAAUCUUCAGCAAC |
| | | GAAAUGGCAAAGGUCGACGACAGCUUCUUCCACAGACUGGAAGA |
| | | AAGCUUCCUGGUCGAAGAAGACAAGAAGCACGAAAGACACCCGA |
| | | UCUUCGGAAACAUCGUCGACGAAGUCGCAUACCACGAAAAGUACC |
| | | CGACAAUCUACCACCUGAGAAAGAAGCUGGUCGACAGCACAGACA |
| | | AGGCAGACCUGAGACUGAUCUACCUGGCACUGGCACACAUGAUCA |
| | | AGUUCAGAGGACACUUCCUGAUCGAAGGAGACCUGAACCCGGAC |
| | | AACAGCGACGUCGACAAGCUGUUCAUCCAGCUGGUCCAGACAUAC |
| | | AACCAGCUGUUCGAAGAAAACCCGAUCAACGCAAGCGGAGUCGAC |
| | | GCAAAGGCAAUCCUGAGCGCAAGACUGAGCAAGAGCAGAAGACU |
| | | GGAAAACCUGAUCGCACAGCUGCCGGGAGAAAAGAAGAACGGAC |
| | | UGUUCGGAAACCUGAUCGCACUGAGCCUGGGACUGACACCGAACU |
| | | UCAAGAGCAACUUCGACCUGGCAGAAGACGCAAAGCUGCAGCUG |
| | | AGCAAGGACACAUACGACGACGACCUGGACAACCUGCUGGCACAG |
| | | AUCGGAGACCAGUACGCAGACCUGUUCCUGGCAGCAAAGAACCUG |
| | | AGCGACGCAAUCCUGCUGAGCGACAUCCUGAGAGUCAACACAGAA |
| | | AUCACAAAGGCACCGCUGAGCGCAAGCAUGAUCAAGAGAUACGA |
| | | CGAACACCACCAGGACCUGACACUGCUGAAGGCACUGGUCAGACA |
| | | GCAGCUGCCGGAAAAGUACAAGGAAAUCUUCUUCGACCAGAGCA |
| | | AGAACGGAUACGCAGGAUACAUCGACGGAGGAGCAAGCCAGGAA |
| | | GAAUUCUACAAGUUCAUCAAGCCGAUCCUGGAAAAGAUGGACGG |
| | | AACAGAAGAACUGCUGGUCAAGCUGAACAGAGAAGACCUGCUGA |
| | | GAAAGCAGAGAACAUUCGACAACGGAAGCAUCCCGCACCAGAUCC |
| | | ACCUGGGAGAACUGCACGCAAUCCUGAGAAGACAGGAAGACUUC |
| | | UACCCGUUCCUGAAGGACAACAGAGAAAAGAUCGAAAAGAUCCU |
| | | GACAUUCAGAAUCCCGUACUACGUCGGACCGCUGGCAAGAGGAA |
| | | ACAGCAGAUUCGCAUGGAUGACAAGAAAGAGCGAAGAAACAAUC |
| | | ACACCGUGGAACUUCGAAGAAGUCGUCGACAAGGGAGCAAGCGC |
| | | ACAGAGCUUCAUCGAAAGAAUGACAAACUUCGACAAGAACCUGC |
| | | CGAACGAAAAGGUCCUGCCGAAGCACAGCCUGCUGUACGAAUACU |
| | | UCACAGUCUACAACGAACUGACAAAGGUCAAGUACGUCACAGAA |
| | | GGAAUGAGAAAGCCGGCAUUCCUGAGCGGAGAACAGAAGAAGGC |
| | | AAUCGUCGACCUGCUGUUCAAGACAAACAGAAAGGUCACAGUCA |
| | | AGCAGCUGAAGGAAGACUACUUCAAGAAGAUCGAAUGCUUCGAC |
| | | AGCGUCGAAAUCAGCGGAGUCGAAGACAGAUUCAACGCAAGCCU |
| | | GGGAACAUACCACGACCUGCUGAAGAUCAUCAAGGACAAGGACU |
| | | UCCUGGACAACGAAGAAAACGAAGACAUCCUGGAAGACAUCGUC |
| | | CUGACACUGACACUGUUCGAAGACAGAGAAAUGAUCGAAGAAAG |
| | | ACUGAAGACAUACGCACACCUGUUCGACGACAAGGUCAUGAAGC |
| | | AGCUGAAGAGAAGAAGAUACACAGGAUGGGGAAGACUGAGCAGA |
| | | AAGCUGAUCAACGGAAUCAGAGACAAGCAGAGCGGAAAGACAAU |
| | | CCUGGACUUCCUGAAGAGCGACGGAUUCGCAAACAGAAACUUCA |
| | | UGCAGCUGAUCCACGACGACAGCCUGACAUUCAAGGAAGACAUCC |
| | | AGAAGGCACAGGUCAGCGGACAGGGAGACAGCCUGCACGAACAC |
| | | AUCGCAAACCUGGCAGGAAGCCCGGCAAUCAAGAAGGGAAUCCU |
| | | GCAGACAGUCAAGGUCGUCGACGAACUGGUCAAGGUCAUGGGAA |
| | | GACACAAGCCGGAAAACAUCGUCAUCGAAAUGGCAAGAGAAAAC |
| | | CAGACAACACAGAAGGGACAGAAGAACAGCAGAGAAAGAAUGAA |
| | | GAGAAUCGAAGAAGGAAUCAAGGAACUGGGAAGCCAGAUCCUGA |
| | | AGGAACACCCGGUCGAAAACACACAGCUGCAGAACGAAAAGCUG |
| | | UACCUGUACUACCUGCAGAACGGAAGAGACAUGUACGUCGACCA |
| | | GGAACUGGACAUCAACAGACUGAGCGACUACGACGUCGACCACAU |
| | | CGUCCCGCAGAGCUUCCUGAAGGACGACAGCAUCGACAACAAGGU |
| | | CCUGACAAGAAGCGACAAGAACAGAGGAAAGAGCGACAACGUCC |
| | | CGAGCGAAGAAGUCGUCAAGAAGAUGAAGAACUACUGGAGACAG |
| | | CUGCUGAACGCAAAGCUGAUCACACAGAGAAAGUUCGACAACCU |
| | | GACAAAGGCAGAGAGAGGAGGACUGAGCGAACUGGACAAGGCAG |
| | | GAUUCAUCAAGAGACAGCUGGUCGAAACAAGACAGAUCACAAAG |
| | | CACGUCGCACAGAUCCUGGACAGCAGAAUGAACACAAAGUACGAC |
| | | GAAAACGACAAGCUGAUCAGAGAAGUCAAGGUCAUCACACUGAA |
| | | GAGCAAGCUGGUCAGCGACUUCAGAAAGGACUUCCAGUUCUACA |
| | | AGGUCAGAGAAAUCAACAACUACCACCACGCACACGACGCAUACC |
| | | UGAACGCAGUCGUCGGAACAGCACUGAUCAAGAAGUACCCGAAG |
| | | CUGGAAAGCGAAUUCGUCUACGGAGACUACAAGGUCUACGACGU |
| | | CAGAAAGAUGAUCGCAAAGAGCGAACAGGAAAUCGGAAAGGCAA |
| | | CAGCAAAGUACUUCUUCUACAGCAACAUCAUGAACUUCUUCAAG |
| | | ACAGAAAUCACACUGGCAAACGGAGAAAUCAGAAAGAGACCGCU |
| | | GAUCGAAACAAACGGAGAAACAGGAGAAAUCGUCUGGGACAAGG |
| | | GAAGAGACUUCGCAACAGUCAGAAAGGUCCUGAGCAUGCCGCAG |
| | | GUCAACAUCGUCAAGAAGACAGAAGUCCAGACAGGAGGAUUCAG |
| | | CAAGGAAAGCAUCCUGCCGAAGAGAAACAGCGACAAGCUGAUCG |
| | | CAAGAAAGAAGGACUGGGACCCGAAGAAGUACGGAGGAUUCGAC |
| | | AGCCCGACAGUCGCAUACAGCGUCCUGGUCGUCGCAAAGGUCGAA |
| | | AAGGGAAAGAGCAAGAAGCUGAAGAGCGUCAAGGAACUGCUGGG |
| | | AAUCACAAUCAUGGAAAGAAGCAGCUUCGAAAAGAACCCGAUCG |
| | | ACUUCCUGGAAGCAAAGGGAUACAAGGAAGUCAAGAAGGACCUG |
| | | AUCAUCAAGCUGCCGAAGUACAGCCUGUUCGAACUGGAAAACGG |
| | | AAGAAAGAGAAUGCUGGCAAGCGCAGGAGAACUGCAGAAGGGAA |
| | | ACGAACUGGCACUGCCGAGCAAGUACGUCAACUUCCUGUACCUGG |
| | | CAAGCCACUACGAAAAGCUGAAGGGAAGCCCGGAAGACAACGAA |
| | | CAGAAGCAGCUGUUCGUCGAACAGCACAAGCACUACCUGGACGAA |
| | | AUCAUCGAACAGAUCAGCGAAUUCAGCAAGAGAGUCAUCCUGGC |
| | | AGACGCAAACCUGGACAAGGUCCUGAGCGCAUACAACAAGCACAG |
| | | AGACAAGCCGAUCAGAGAACAGGCAGAAAACAUCAUCCACCUGU |
| | | UCACACUGACAAACCUGGGAGCACCGGCAGCAUUCAAGUACUUCG |
| | | ACACAACAAUCGACAGAAAGAGAUACACAAGCACAAAGGAAGUC |
| | | CUGGACGCAACACUGAUCCACCAGAGCAUCACAGGACUGUACGAA |
| | | ACAAGAAUCGACCUGAGCCAGCUGGGAGGAGACGGAGGAGGAAG |
| | | CCCGAAGAAGAAGAGAAAGGUCUAG |
| 294 | Amino acid | MDKKYSIGLDIGTNSVGWAVITDEYKVPSKKFKVLGNTDRHSIKKNLI |
| | sequence for Cas9 | GALLFDSGETAEATRLKRTARRRYTRRKNRICYLQEIFSNEMAKVDDS |
| | | FFHRLEESFLVEEDKKHERHPIFGNIVDEVAYHEKYPTIYHLRKKLVDS |
| | | TDKADLRLIYLALAHMIKFRGHFLIEGDLNPDNSDVDKLFIQLVQTYN |
| | | QLFEENPINASGVDAKAILSARLSKSRRLENLIAQLPGEKKNGLFGNLIA |
| | | LSLGLTPNFKSNFDLAEDAKLQLSKDTYDDDLDNLLAQIGDQYADLFL |
| | | AAKNLSDAILLSDILRVNTEITKAPLSASMIKRYDEHHQDLTLLKALVR |
| | | QQLPEKYKEIFFDQSKNGYAGYIDGGASQEEFYKFIKPILEKMDGTEEL |
| | | LVKLNREDLLRKQRTFDNGSIPHQIHLGELHAILRRQEDFYPFLKDNRE |
| | | KIEKILTFRIPYYVGPLARGNSRFAWMTRKSEETITPWNFEEVVDKGAS |
| | | AQSFIERMTNFDKNLPNEKVLPKHSLLYEYFTVYNELTKVKYVTEGMR |
| | | KPAFLSGEQKKAIVDLLFKTNRKVTVKQLKEDYFKKIECFDSVEISGVE |
| | | DRFNASLGTYHDLLKIIKDKDFLDNEENEDILEDIVLTLTLFEDREMIEE |
| | | RLKTYAHLFDDKVMKQLKRRRYTGWGRLSRKLINGIRDKQSGKTILD |
| | | FLKSDGFANRNFMQLIHDDSLTFKEDIQKAQVSGQGDSLHEHIANLAG |
| | | SPAIKKGILQTVKVVDELVKVMGRHKPENIVIEMARENQTTQKGQKNS |
| | | RERMKRIEEGIKELGSQILKEHPVENTQLQNEKLYLYYLQNGRDMYVD |
| | | QELDINRLSDYDVDHIVPQSFLKDDSIDNKVLTRSDKNRGKSDNVPSEE |
| | | VVKKMKNYWRQLLNAKLITQRKFDNLTKAERGGLSELDKAGFIKRQL |
| | | VETRQITKHVAQILDSRMNTKYDENDKLIREVKVITLKSKLVSDFRKDF |
| | | QFYKVREINNYHHAHDAYLNAVVGTALIKKYPKLESEFVYGDYKVYD |
| | | VRKMIAKSEQEIGKATAKYFFYSNIMNFFKTEITLANGEIRKRPLIETNG |
| | | ETGEIVWDKGRDFATVRKVLSMPQVNIVKKTEVQTGGFSKESILPKRN |
| | | SDKLIARKKDWDPKKYGGFDSPTVAYSVLVVAKVEKGKSKKLKSVKE |
| | | LLGITIMERSSFEKNPIDFLEAKGYKEVKKDLIIKLPKYSLFELENGRKR |
| | | MLASAGELQKGNELALPSKYVNFLYLASHYEKLKGSPEDNEQKQLFV |
| | | EQHKHYLDEIIEQISEFSKRVILADANLDKVLSAYNKHRDKPIREQAENI |
| | | IHLFTLTNLGAPAAFKYFDTTIDRKRYTSTKEVLDATLIHQSITGLYETR |
| | | IDLSQLGGDGGGSPKKKRKV |
| 295 | Open reading | AUGGACAAGAAGUACUCCAUCGGCCUGGACAUCGGCACCAACUCC |
| | frame for Cas9 | GUGGGCUGGGCCGUGAUCACCGACGAGUACAAGGUGCCCUCCAAG |
| | | AAGUUCAAGGUGCUGGGCAACACCGACCGGCACUCCAUCAAGAAG |
| | | AACCUGAUCGGCGCCCUGCUGUUCGACUCCGGCGAGACCGCCGAG |
| | | GCCACCCGGCUGAAGCGGACCGCCCGGCGGCGGUACACCCGGCGG |
| | | AAGAACCGGAUCUGCUACCUGCAGGAGAUCUUCUCCAACGAGAU |
| | | GGCCAAGGUGGACGACUCCUUCUUCCACCGGCUGGAGGAGUCCUU |
| | | CCUGGUGGAGGAGGACAAGAAGCACGAGCGGCACCCCAUCUUCGG |
| | | CAACAUCGUGGACGAGGUGGCCUACCACGAGAAGUACCCCACCAU |
| | | CUACCACCUGCGGAAGAAGCUGGUGGACUCCACCGACAAGGCCGA |
| | | CCUGCGGCUGAUCUACCUGGCCCUGGCCCACAUGAUCAAGUUCCG |
| | | GGGCCACUUCCUGAUCGAGGGCGACCUGAACCCCGACAACUCCGA |
| | | CGUGGACAAGCUGUUCAUCCAGCUGGUGCAGACCUACAACCAGCU |
| | | GUUCGAGGAGAACCCCAUCAACGCCUCCGGCGUGGACGCCAAGGC |
| | | CAUCCUGUCCGCCCGGCUGUCCAAGUCCCGGCGGCUGGAGAACCU |
| | | GAUCGCCCAGCUGCCCGGCGAGAAGAAGAACGGCCUGUUCGGCAA |
| | | CCUGAUCGCCCUGUCCCUGGGCCUGACCCCCAACUUCAAGUCCAA |
| | | CUUCGACCUGGCCGAGGACGCCAAGCUGCAGCUGUCCAAGGACAC |
| | | CUACGACGACGACCUGGACAACCUGCUGGCCCAGAUCGGCGACCA |
| | | GUACGCCGACCUGUUCCUGGCCGCCAAGAACCUGUCCGACGCCAU |
| | | CCUGCUGUCCGACAUCCUGCGGGUGAACACCGAGAUCACCAAGGC |
| | | CCCCCUGUCCGCCUCCAUGAUCAAGCGGUACGACGAGCACCACCA |
| | | GGACCUGACCCUGCUGAAGGCCCUGGUGCGGCAGCAGCUGCCCGA |
| | | GAAGUACAAGGAGAUCUUCUUCGACCAGUCCAAGAACGGCUACG |
| | | CCGGCUACAUCGACGGCGGCGCCUCCCAGGAGGAGUUCUACAAGU |
| | | UCAUCAAGCCCAUCCUGGAGAAGAUGGACGGCACCGAGGAGCUGC |
| | | UGGUGAAGCUGAACCGGGAGGACCUGCUGCGGAAGCAGCGGACC |
| | | UUCGACAACGGCUCCAUCCCCCACCAGAUCCACCUGGGCGAGCUG |
| | | CACGCCAUCCUGCGGCGGCAGGAGGACUUCUACCCCUUCCUGAAG |
| | | GACAACCGGGAGAAGAUCGAGAAGAUCCUGACCUUCCGGAUCCCC |
| | | UACUACGUGGGCCCCCUGGCCCGGGGCAACUCCCGGUUCGCCUGG |
| | | AUGACCCGGAAGUCCGAGGAGACCAUCACCCCCUGGAACUUCGAG |
| | | GAGGUGGUGGACAAGGGCGCCUCCGCCCAGUCCUUCAUCGAGCGG |
| | | AUGACCAACUUCGACAAGAACCUGCCCAACGAGAAGGUGCUGCCC |
| | | AAGCACUCCCUGCUGUACGAGUACUUCACCGUGUACAACGAGCUG |
| | | ACCAAGGUGAAGUACGUGACCGAGGGCAUGCGGAAGCCCGCCUUC |
| | | CUGUCCGGCGAGCAGAAGAAGGCCAUCGUGGACCUGCUGUUCAA |
| | | GACCAACCGGAAGGUGACCGUGAAGCAGCUGAAGGAGGACUACU |
| | | UCAAGAAGAUCGAGUGCUUCGACUCCGUGGAGAUCUCCGGCGUG |
| | | GAGGACCGGUUCAACGCCUCCCUGGGCACCUACCACGACCUGCUG |
| | | AAGAUCAUCAAGGACAAGGACUUCCUGGACAACGAGGAGAACGA |
| | | GGACAUCCUGGAGGACAUCGUGCUGACCCUGACCCUGUUCGAGGA |
| | | CCGGGAGAUGAUCGAGGAGCGGCUGAAGACCUACGCCCACCUGUU |
| | | CGACGACAAGGUGAUGAAGCAGCUGAAGCGGCGGCGGUACACCG |
| | | GCUGGGGCCGGCUGUCCCGGAAGCUGAUCAACGGCAUCCGGGACA |
| | | AGCAGUCCGGCAAGACCAUCCUGGACUUCCUGAAGUCCGACGGCU |
| | | UCGCCAACCGGAACUUCAUGCAGCUGAUCCACGACGACUCCCUGA |
| | | CCUUCAAGGAGGACAUCCAGAAGGCCCAGGUGUCCGGCCAGGGCG |
| | | ACUCCCUGCACGAGCACAUCGCCAACCUGGCCGGCUCCCCCGCCA |
| | | UCAAGAAGGGCAUCCUGCAGACCGUGAAGGUGGUGGACGAGCUG |
| | | GUGAAGGUGAUGGGCCGGCACAAGCCCGAGAACAUCGUGAUCGA |
| | | GAUGGCCCGGGAGAACCAGACCACCCAGAAGGGCCAGAAGAACUC |
| | | CCGGGAGCGGAUGAAGCGGAUCGAGGAGGGCAUCAAGGAGCUGG |
| | | GCUCCCAGAUCCUGAAGGAGCACCCCGUGGAGAACACCCAGCUGC |
| | | AGAACGAGAAGCUGUACCUGUACUACCUGCAGAACGGCCGGGAC |
| | | AUGUACGUGGACCAGGAGCUGGACAUCAACCGGCUGUCCGACUAC |
| | | GACGUGGACCACAUCGUGCCCCAGUCCUUCCUGAAGGACGACUCC |
| | | AUCGACAACAAGGUGCUGACCCGGUCCGACAAGAACCGGGGCAAG |
| | | UCCGACAACGUGCCCUCCGAGGAGGUGGUGAAGAAGAUGAAGAA |
| | | CUACUGGCGGCAGCUGCUGAACGCCAAGCUGAUCACCCAGCGGAA |
| | | GUUCGACAACCUGACCAAGGCCGAGCGGGGCGGCCUGUCCGAGCU |
| | | GGACAAGGCCGGCUUCAUCAAGCGGCAGCUGGUGGAGACCCGGCA |
| | | GAUCACCAAGCACGUGGCCCAGAUCCUGGACUCCCGGAUGAACAC |
| | | CAAGUACGACGAGAACGACAAGCUGAUCCGGGAGGUGAAGGUGA |
| | | UCACCCUGAAGUCCAAGCUGGUGUCCGACUUCCGGAAGGACUUCC |
| | | AGUUCUACAAGGUGCGGGAGAUCAACAACUACCACCACGCCCACG |
| | | ACGCCUACCUGAACGCCGUGGUGGGCACCGCCCUGAUCAAGAAGU |
| | | ACCCCAAGCUGGAGUCCGAGUUCGUGUACGGCGACUACAAGGUG |
| | | UACGACGUGCGGAAGAUGAUCGCCAAGUCCGAGCAGGAGAUCGG |
| | | CAAGGCCACCGCCAAGUACUUCUUCUACUCCAACAUCAUGAACUU |
| | | CUUCAAGACCGAGAUCACCCUGGCCAACGGCGAGAUCCGGAAGCG |
| | | GCCCCUGAUCGAGACCAACGGCGAGACCGGCGAGAUCGUGUGGGA |
| | | CAAGGGCCGGGACUUCGCCACCGUGCGGAAGGUGCUGUCCAUGCC |
| | | CCAGGUGAACAUCGUGAAGAAGACCGAGGUGCAGACCGGCGGCU |
| | | UCUCCAAGGAGUCCAUCCUGCCCAAGCGGAACUCCGACAAGCUGA |
| | | UCGCCCGGAAGAAGGACUGGGACCCCAAGAAGUACGGCGGCUUCG |
| | | ACUCCCCCACCGUGGCCUACUCCGUGCUGGUGGUGGCCAAGGUGG |
| | | AGAAGGGCAAGUCCAAGAAGCUGAAGUCCGUGAAGGAGCUGCUG |
| | | GGCAUCACCAUCAUGGAGCGGUCCUCCUUCGAGAAGAACCCCAUC |
| | | GACUUCCUGGAGGCCAAGGGCUACAAGGAGGUGAAGAAGGACCU |
| | | GAUCAUCAAGCUGCCCAAGUACUCCCUGUUCGAGCUGGAGAACGG |
| | | CCGGAAGCGGAUGCUGGCCUCCGCCGGCGAGCUGCAGAAGGGCAA |
| | | CGAGCUGGCCCUGCCCUCCAAGUACGUGAACUUCCUGUACCUGGC |
| | | CUCCCACUACGAGAAGCUGAAGGGCUCCCCCGAGGACAACGAGCA |
| | | GAAGCAGCUGUUCGUGGAGCAGCACAAGCACUACCUGGACGAGA |
| | | UCAUCGAGCAGAUCUCCGAGUUCUCCAAGCGGGUGAUCCUGGCCG |
| | | ACGCCAACCUGGACAAGGUGCUGUCCGCCUACAACAAGCACCGGG |
| | | ACAAGCCCAUCCGGGAGCAGGCCGAGAACAUCAUCCACCUGUUCA |
| | | CCCUGACCAACCUGGGCGCCCCCGCCGCCUUCAAGUACUUCGACA |
| | | CCACCAUCGACCGGAAGCGGUACACCUCCACCAAGGAGGUGCUGG |
| | | ACGCCACCCUGAUCCACCAGUCCAUCACCGGCCUGUACGAGACCC |
| | | GGAUCGACCUGUCCCAGCUGGGCGGCGACGGCGGCGGCUCCCCCA |
| | | AGAAGAAGCGGAAGGUGUGA |
| 296 | Amino acid | MDKKYSIGLDIGTNSVGWAVITDEYKVPSKKFKVLGNTDRHSIKKNLI |
| | sequence for | GALLFDSGETAEATRLKRTARRRYTRRKNRICYLQEIFSNEMAKVDDS |
| | Cas9-NLS | FFHRLEESFLVEEDKKHERHPIFGNIVDEVAYHEKYPTIYHLRKKLVDS |
| | | TDKADLRLIYLALAHMIKFRGHFLIEGDLNPDNSDVDKLFIQLVQTYN |
| | | QLFEENPINASGVDAKAILSARLSKSRRLENLIAQLPGEKKNGLFGNLIA |
| | | LSLGLTPNFKSNFDLAEDAKLQLSKDTYDDDLDNLLAQIGDQYADLFL |
| | | AAKNLSDAILLSDILRVNTEITKAPLSASMIKRYDEHHQDLTLLKALVR |
| | | QQLPEKYKEIFFDQSKNGYAGYIDGGASQEEFYKFIKPILEKMDGTEEL |
| | | LVKLNREDLLRKQRTFDNGSIPHQIHLGELHAILRRQEDFYPFLKDNRE |
| | | KIEKILTFRIPYYVGPLARGNSRFAWMTRKSEETTTPWNFEEVVDKGAS |
| | | AQSFIERMTNFDKNLPNEKVLPKHSLLYEYFTVYNELTKVKYVTEGMR |
| | | KPAFLSGEQKKAIVDLLFKTNRKVTVKQLKEDYFKKIECFDSVEISGVE |
| | | DRFNASLGTYHDLLKIIKDKDFLDNEENEDILEDIVLTLTLFEDREMIEE |
| | | RLKTYAHLFDDKVMKQLKRRRYTGWGRLSRKLINGIRDKQSGKTILD |
| | | FLKSDGFANRNFMQLIHDDSLTFKEDIQKAQVSGQGDSLHEHIANLAG |
| | | SPAIKKGILQTVKVVDELVKVMGRHKPENIVIEMARENQTTQKGQKNS |
| | | RERMKRIEEGIKELGSQILKEHPVENTQLQNEKLYLYYLQNGRDMYVD |
| | | QELDINRLSDYDVDHIVPQSFLKDDSIDNKVLTRSDKNRGKSDNVPSEE |
| | | VVKKMKNYWRQLLNAKLITQRKFDNLTKAERGGLSELDKAGFIKRQL |
| | | VETRQITKHVAQILDSRMNTKYDENDKLIREVKVITLKSKLVSDFRKDF |
| | | QFYKVREINNYHHAHDAYLNAVVGTALIKKYPKLESEFVYGDYKVYD |
| | | VRKMIAKSEQEIGKATAKYFFYSNIMNFFKTEITLANGEIRKRPLIETNG |
| | | ETGEIVWDKGRDFATVRKVLSMPQVNIVKKTEVQTGGFSKESILPKRN |
| | | SDKLIARKKDWDPKKYGGFDSPTVAYSVLVVAKVEKGKSKKLKSVKE |
| | | LLGITIMERSSFEKNPIDFLEAKGYKEVKKDLIIKLPKYSLFELENGRKR |
| | | MLASAGELQKGNELALPSKYVNFLYLASHYEKLKGSPEDNEQKQLFV |
| | | EQHKHYLDEIIEQISEFSKRVILADANLDKVLSAYNKHRDKPIREQAENI |
| | | IHLFTLTNLGAPAAFKYFDTTIDRKRYTSTKEVLDATLIHQSITGLYETR |
| | | IDLSQLGGDGGGSPKKKRKV |
| 297 | TCR insertion | TTGGCCACTCCCTCTCTGCGCGCTCGCTCGCTCACTGAGGCCGGGCG |
| | construct with | ACCAAAGGTCGCCCGACGCCCGGGCTTTGCCCGGGCGGCCTCAGTG |
| | homology arms | AGCGAGCGAGCGCGCAGAGAGGGAGTGGCCAACTCCATCACTAGG |
| | flanking TRAC | GGTTCCTagatcttgccaacataccataaacctcccattctgctaatgcccagcctaagttggggagaccac |
| | G013006 cut site - | tccagattccaagatgtacagtttgctttgctgggcctttttcccatgcctgcctttactctgccagagttatattgctg |
| | ITR included | gggttttgaagaagatcctattaaataaaagaataagcagtattattaagtagccctgcatttcaggtttccttgagtg |
| | | gcaggccaggcctggccgtgaacgttcactgaaatcatggcctcttggccaagattgatagcttgtgcctgtccct |
| | | gagtcccagtccatcacgagcagctggtttctaagatgctatttcccgtataaagcatgagaccgtgacttgccag |
| | | ccccacagagccccgcccttgtccatcactggcatctggactccagcctgggttggggcaaagagggaaatga |
| | | gatcatgtcctaaccctgatcctcttgtcccacagATATCCAGAACCCTGACCCTGCGGCT |
| | | CCGGTGCCCGTCAGTGGGCAGAGCGCACATCGCCCACAGTCCCCGA |
| | | GAAGTTGGGGGGAGGGGTCGGCAATTGAACCGGTGCCTAGAGAAG |
| | | GTGGCGCGGGGTAAACTGGGAAAGTGATGTCGTGTACTGGCTCCGC |
| | | CTTTTTCCCGAGGGTGGGGGAGAACCGTATATAAGTGCAGTAGTCG |
| | | CCGTGAACGTTCTTTTTCGCAACGGGTTTGCCGCCAGAACACAGGT |
| | | AAGTGCCGTGTGTGGTTCCCGCGGGCCTGGCCTCTTTACGGGTTATG |
| | | GCCCTTGCGTGCCTTGAATTACTTCCACGCCCCTGGCTGCAGTACGT |
| | | GATTCTTGATCCCGAGCTTCGGGTTGGAAGTGGGTGGGAGAGTTCG |
| | | AGGCCTTGCGCTTAAGGAGCCCCTTCGCCTCGTGCTTGAGTTGAGG |
| | | CCTGGCTTGGGCGCTGGGGCCGCCGCGTGCGAATCTGGTGGCACCT |
| | | TCGCGCCTGTCTCGCTGCTTTCGATAAGTCTCTAGCCATTTAAAATT |
| | | TTTGATGACCTGCTGCGACGCTTTTTTTCTGGCAAGATAGTCTTGTA |
| | | AATGCGGGCCAAGATgTGCACACTGGTATTTCGGTTTTTGGGGCCGC |
| | | GGGCGGCGACGGGGCCCGTGCGTCCCAGCGCACATGTTCGGCGAG |
| | | GCGGGGCCTGCGAGCGCGGCCACCGAGAATCGGACGGGGGTAGTC |
| | | TCAAGCTGGCCGGCCTGCTCTGGTGCCTGGCCTCGCGCCGCCGTGT |
| | | ATCGCCCCGCCCTGGGCGGCAAGGCTGGCCCGGTCGGCACCAGTTG |
| | | CGTGAGCGGAAAGATGGCCGCTTCCCGGCCCTGCTGCAGGGAGCTC |
| | | AAAATGGAGGACGCGGCGCTCGGGAGAGCGGGCGGGTGAGTCACC |
| | | CACACAAAGGAAAAGGGCCTTTCCGTCCTCAGCCGTCGCTTCATGT |
| | | GACTCCACGGAGTACCGGGCGCCGTCCAGGCACCTCGATTAGTTCT |
| | | CGAGCTTTTGGAGTACGTCGTCTTTAGGTTGGGGGGAGGGGTTTTAT |
| | | GCGATGGAGTTTCCCCACACTGAGTGGGTGGAGACTGAAGTTAGGC |
| | | CAGCTTGGCACTTGATGTAATTCTCCTTGGAATTTGCCCTTTTTGAG |
| | | TTTGGATCTTGGTTCATTCTCAAGCCTCAGACAGTGGTTCAAAGTTT |
| | | TTTTCTTCCATTTCAGGTGTCGTGAtGCGGCCGCCACCATGGCCCTGC |
| | | CCGTGACCGCCCTGCTGCTGCCCCTGGCCCTGCTGCTGCACGCCGCC |
| | | CGGCCCCAGGTGCAGCTGGTGGAGAGCGGCGGCGGCCTGGTGCAG |
| | | CCCGGCGGCAGCCTGCGGCTGAGCTGCGTGGCCAGCGGCTTCACCT |
| | | TCAGCAGCAACGCCATGAGCTGGGTGCGGCAGGCCCCCGGCAAGG |
| | | GCCTGGAGTGGGTGAGCGCCATCAGCGGCAGCGGCGACTACACCC |
| | | ACTACAGCGACAGCGTGAAGGGCCGGTTCACCATCAGCCGGGACA |
| | | ACAGCAAGAACACCCTGTACCTGCAGATGAACAGCCTGCGGGCCG |
| | | AGGACACCGCCGTGTACTACTGCGCCAAGGAGGTGCCCGGCGGCCC |
| | | CCTGGTGGACTTCGACAGCCGGGGCCAGGGCACCCTGGTGACCGTG |
| | | AGCAGCGGCGGCGGCGGCAGCCAGGTGCAGCTGGTGGAGAGCGGC |
| | | GGCGGCCTGGTGCAGCCCGGCGGCAGCCTGCGGCTGAGCTGCGTGG |
| | | CCAGCGGCTTCACCTTCAGCAGCAACGCCATGAGCTGGGTGCGGCA |
| | | GGCCCCCGGCAAGGGCCTGGAGTGGGTGAGCGCCATCAGCGGCAG |
| | | CGGCGACTACACCCACTACAGCGACAGCGTGAAGGGCCGGTTCACC |
| | | ATCAGCCGGGACAACAGCAAGAACACCCTGTACCTGCAGATGAAC |
| | | AGCCTGCGGGCCGAGGACACCGCCGTGTACTACTGCGCCAAGGAG |
| | | GTGCCCGGCGGCCCCCTGGTGGACTTCGACAGCCGGGGCCAGGGCA |
| | | CCCTGGTGACCGTGAGCAGCACCACCACCCCCGCCCCCCGGCCCCC |
| | | CACCCCCGCCCCCACCATCGCCAGCCAGCCCCTGAGCCTGCGGCCC |
| | | GAGGCCTGCCGGCCCGCCGCCGGCGGCGCCGTGCACACCCGGGGCC |
| | | TGGACTTCGCCTGCGACTTCTGGGTGCTGGTGGTGGTGGGCGGCGT |
| | | GCTGGCCTGCTACAGCCTGCTGGTGACCGTGGCCTTCATCATCTTCT |
| | | GGGTGCGGAGCAAGCGGAGCCGGCTGCTGCACAGCGACTACATGA |
| | | ACATGACCCCCCGGCGGCCCGGCCCCACCCGGAAGCACTACCAGCC |
| | | CTACGCCCCCCCCCGGGACTTCGCCGCCTACCGGAGCCGGGTGAAG |
| | | TTCAGCCGGAGCGCCGACGCCCCCGCCTACCAGCAGGGCCAGAACC |
| | | AGCTGTACAACGAGCTGAACCTGGGCCGGCGGGAGGAGTACGACG |
| | | TGCTGGACAAGCGGCGGGGCCGGGACCCCGAGATGGGCGGCAAGC |
| | | CCCGGCGGAAGAACCCCCAGGAGGGCCTGTACAACGAGCTGCAGA |
| | | AGGACAAGATGGCCGAGGCCTACAGCGAGATCGGCATGAAGGGCG |
| | | AGCGGCGGCGGGGCAAGGGCCACGACGGCCTGTACCAGGGCCTGA |
| | | GCACCGCCACCAAGGACACCTACGACGCCCTGCACATGCAGGCCCT |
| | | GCCCCCCCGGTAATGACCTCGACTGTGCCTTCTAGTTGCCAGCCATC |
| | | TGTTGTTTGCCCCTCCCCCGTGCCTTCCTTGACCCTGGAAGGTGCCA |
| | | CTCCCACTGTCCTTTCCTAATAAAATGAGGAAATTGCATCGCATTGT |
| | | CTGAGTAGGTGTCATTCTATTCTGGGGGGTGGGGTGGGGCAGGACA |
| | | GCAAGGGGGAGGATTGGGAAGACAATAGCAGGCATGCTGGGGATG |
| | | CGGTGGGCTCTATGGCTTCTGAGGCGGAAAGAACCAGCTGGGGCTC |
| | | TAGGGGGTATCCCCACTAGTCGTGTACCAGCTGAGAGACTCTAAAT |
| | | CCAGTGACAAGTCTGTCTGCCTATTCACCGATTTTGATTCTCAAACA |
| | | AATGTGTCACAAAGTAAGGATTCTGATGTGTATATCACAGACAAAA |
| | | CTGTGCTAGACATGAGGTCTATGGACTTCAAGAGCAACAGTGCTGT |
| | | GGCCTGGAGCAACAAATCTGACTTTGCATGTGCAAACGCCTTCAAC |
| | | AACAGCATTATTCCAGAAGACACCTTCTTCCCCAGCCCAGgtaagggcag |
| | | ctttggtgccttcgcaggctgtttccttgcttcaggaatggccaggttctgcccagagctctggtcaatgatgtctaa |
| | | aactcctctgattggtggtctcggccttatccattgccaccaaaaccctctttttactaagaaacagtgagccttgttc |
| | | tggcagtccagagaatgacacgggaaaaaagcagatgaagagaaggtggcaggagagggcacgtggccca |
| | | gcctcagtctctAGATCTAGGAACCCCTAGTGATGGAGTTGGCCACTCCCT |
| | | CTCTGCGCGCTCGCTCGCTCACTGAGGCCGCCCGGGCAAAGCCCGG |
| | | GCGTCGGGCGACCTTTGGTCGCCCGGCCTCAGTGAGCGAGCGAGCG |
| | | CGCAGAGAGGGAGTGGCCAA |
| 298 | bidirectional | taggtcagtgaagagaagaacaaaaagcagcatattacagttagttgtcttcatcaatctttaaatatgttgtgtggtt |
| | SERPINA | tttctctccctgtttccacagttGAGGACCCCCAGGGCGACGCCGCCCAGAAGACC |
| | insertion | GACACCAGCCACCACGACCAGGACCACCCCACCTTCAACAAGATCA |
| | construct | CCCCCAACCTGGCCGAGTTCGCCTTCAGCCTGTACAGGCAGCTGGC |
| | | CCACCAGAGCAACAGCACCAACATCTTCTTCAGCCCCGTGAGCATC |
| | | GCCACCGCCTTCGCCATGCTGAGCCTGGGCACCAAGGCCGACACCC |
| | | ACGACGAGATCCTGGAGGGCCTGAACTTCAACCTGACCGAGATCCC |
| | | CGAGGCCCAGATCCACGAGGGCTTCCAGGAGCTGCTGAGGACCCTG |
| | | AACCAGCCCGACAGCCAGCTGCAGCTGACCACCGGCAACGGCCTGT |
| | | TCCTGAGCGAGGGCCTGAAGCTGGTGGACAAGTTCCTGGAGGACGT |
| | | GAAGAAGCTGTACCACAGCGAGGCCTTCACCGTGAACTTCGGCGAC |
| | | ACCGAGGAGGCCAAGAAGCAGATCAACGACTACGTGGAGAAGGGC |
| | | ACCCAGGGCAAGATCGTGGACCTGGTGAAGGAGCTGGACAGGGAC |
| | | ACCGTGTTCGCCCTGGTGAACTACATCTTCTTCAAGGGCAAGTGGG |
| | | AGAGGCCCTTCGAGGTGAAGGACACCGAGGAGGAGGACTTCCACG |
| | | TGGACCAGGTGACCACCGTGAAGGTGCCCATGATGAAGAGGCTGG |
| | | GCATGTTCAACATCCAGCACTGCAAGAAGCTGAGCAGCTGGGTGCT |
| | | GCTGATGAAGTACCTGGGCAACGCCACCGCCATCTTCTTCCTGCCC |
| | | GACGAGGGCAAGCTGCAGCACCTGGAGAACGAGCTGACCCACGAC |
| | | ATCATCACCAAGTTCCTGGAGAACGAGGACAGGAGGAGCGCCAGC |
| | | CTGCACCTGCCCAAGCTGAGCATCACCGGCACCTACGACCTGAAGA |
| | | GCGTGCTGGGCCAGCTGGGCATCACCAAGGTGTTCAGCAACGGCGC |
| | | CGACCTGAGCGGCGTGACCGAGGAGGCCCCCCTGAAGCTGAGCAA |
| | | GGCCGTGCACAAGGCCGTGCTGACCATCGACGAGAAGGGCACCGA |
| | | GGCCGCCGGCGCCATGTTCCTGGAGGCCATCCCCATGAGCATCCCC |
| | | CCCGAGGTGAAGTTCAACAAGCCCTTCGTGTTCCTGATGATCGAGC |
| | | AGAACACCAAGAGCCCCCTGTTCATGGGCAAGGTGGTGAACCCCAC |
| | | CCAGAAGTAACAGACATGATAAGATACATTGATGAGTTTGGACAAA |
| | | CCACAACTAGAATGCAGTGAAAAAAATGCTTTATTTGTGAAATTTG |
| | | TGATGCTATTGCTTTATTTGTAACCATTATAAGCTGCAATAAACAAG |
| | | TTAACAACAACAATTGCATTCATTTTATGTTTCAGGTTCAGGGGGAG |
| | | GTGTGGGAGGTTTTTTggggataccccctagagccccagctggttctttccgcctcagaagCC |
| | | ATAGAGCCCACCGCATCCCCAGCATGCCTGCTATTGTCTTCCCAATC |
| | | CTCCCCCTTGCTGTCCTGCCCCACCCCACCCCCCAGAATAGAATGAC |
| | | ACCTACTCAGACAATGCGATGCAATTTCCTCATTTTATTAGGAAAG |
| | | GACAGTGGGAGTGGCACCTTCCAGGGTCAAGGAAGGCACGGGGGA |
| | | GGGGCAAACAACAGATGGCTGGCAACTAGAAGGCACAGTCGaggttaT |
| | | TTTTGGGTGGGATTCACCACTTTTCCCATGAAGAGGGGAGACTTGG |
| | | TATTTTGTTCAATCATTAAGAAGACAAAGGGTTTGTTGAACTTGACC |
| | | TCGGGGGGGATAGACATGGGTATGGCCTCTAAAAACATGGCCCCAG |
| | | CAGCTTCAGTCCCTTTCTCGTCGATGGTCAGCACAGCCTTATGCACG |
| | | GCCTTGGAGAGCTTCAGGGGTGCCTCCTCTGTGACCCCGGAGAGGT |
| | | CAGCCCCATTGCTGAAGACCTTAGTGATGCCCAGTTGACCCAGGAC |
| | | GCTCTTCAGATCATAGGTTCCAGTAATGGACAGTTTGGGTAAATGT |
| | | AAGCTGGCAGACCTTCTGTCTTCATTTTCCAGGAACTTGGTGATGAT |
| | | ATCGTGGGTGAGTTCATTTTCCAGGTGCTGTAGTTTCCCCTCATCAG |
| | | GCAGGAAGAAGATGGCGGTGGCATTGCCCAGGTATTTCATCAGCAG |
| | | CACCCAGCTGGACAGCTTCTTACAGTGCTGGATGTTAAACATGCCT |
| | | AAACGCTTCATCATAGGCACCTTCACGGTGGTCACCTGGTCCACGT |
| | | GGAAGTCCTCTTCCTCGGTGTCCTTGACTTCAAAGGGTCTCTCCCAT |
| | | TTGCCTTTAAAGAAGATGTAATTCACCAGAGCAAAAACTGTGTCTC |
| | | TGTCAAGCTCCTTGACCAAATCCACAATTTTCCCTTGAGTACCCTTC |
| | | TCCACGTAATCGTTGATCTGTTTCTTGGCCTCTTCGGTGTCCCCGAA |
| | | GTTGACAGTGAAGGCTTCTGAGTGGTACAACTTTTTAACATCCTCCA |
| | | AAAACTTATCCACTAGCTTCAGGCCCTCGCTGAGGAACAGGCCATT |
| | | GCCGGTGGTCAGCTGGAGCTGGCTGTCTGGCTGGTTGAGGGTACGG |
| | | AGGAGTTCCTGGAAGCCTTCATGGATCTGAGCCTCCGGAATCTCCG |
| | | TGAGGTTGAAATTCAGGCCCTCCAGGATTTCATCGTGAGTGTCAGC |
| | | CTTGGTCCCCAGGGAGAGCATTGCAAAGGCTGTAGCGATGCTCACT |
| | | GGGGAGAAGAAGATATTGGTGCTGTTGGACTGGTGTGCCAGCTGGC |
| | | GGTATAGGCTGAAGGCGAACTCAGCCAGGTTGGGGGTGATCTTGTT |
| | | GAAGGTTGGGTGATCCTGATCATGGTGGGATGTATCTGTCTTCTGG |
| | | GCAGCATCTCCCTGGGGATCCTCaactgtggaaacagggagagaaaaaccacacaacat |
| | | atttaaagattgatgaagacaactaactgtaatatgctgctttttgttcttctcttcactgaccta |
| 299 | Template A eGFP | tggccctggctttggcagcctgtgctgacccatgcagtcctccttaccatccctccctcgacttcccctcttccgatg |
| | insertion | ttgagcccctccagccggtcctggactttgtctccttccctgccctgccctctcctgaacctgagccagctcccata |
| | construct with | gctcagtctggtctatctgcctggccctggccattgtcactttgcgctgccctcctctcgcccccgagtgcccttgct |
| | homology arms to | gtgccgccggaactctgccctctaacgctgccgtctctctcctgagtccggaccactttgagctctactggcttctg |
| | mouse AAVS1 | cgccgcctctggcccactgtttccccttcccaggcaggtcctgctttctctgacctgcattctctcccctgggcctgt |
| | | gccgctttctgtctgcagcttgtggcctgggtcacctctacggctggcccagatccttccctgccgcctccttcagg |
| | | ttccgtcttcctccactccctcttccccttgctctctgctgtgttgctgcccaaggatgctctttccggagcacttcctt |
| | | ctcggcgctgcaccacgtgatgtcctctgagcggatcctccccgtgtctgggtcctctccgggcatctctcctccc |
| | | tcacccaaccccatgccgtcttcactcgctgggttcccttttccttctccttctggggcctgtgccatctctcgtttctt |
| | | aggatggccttctccgacggatgtctcccttgcgtcccgcctccccttcttgtaggcctgcatcatcaccgtttttct |
| | | ggacaaccccaaagtaccccgtctccctggctttagccaccttccatcctcttgctttctttgcctggacaccccgt |
| | | tctcctgtggattcgggtcacctctcactcctttcatttgggcagctcccctaccccccttacctctctagtctgtgcta |
| | | gctcttccagccctagttattaatgagtaattcatacaaaaggactcgcccctgccttggggaatcccagggaccg |
| | | tcgttaaactcccactaacgtagaacccagagatcgctgcgttcccgccccctcacccgcccgctctcgtcatca |
| | | ctgaggtggagaagagcatgcgtgaggctccggtgcccgtcagtgggcagagcgcacatcgcccacagtccc |
| | | cgagaagttggggggaggggtcggcaattgaaccggtgcctagagaaggtggcgcggggtaaactgggaaa |
| | | gtgatgtcgtgtactggctccgcctttttcccgaggggggggagaaccgtatataagtgcagtagtcgccgtga |
| | | acgttctttttcgcaacgggtttgccgccagaacacaggtaagtgccgtgtgtggttcccgcgggcctggcctcttt |
| | | acgggttatggcccttgcgtgccttgaattacttccacgcccctggctgcagtacgtgattcttgatcccgagcttc |
| | | gggttggaagtgggtgggagagttcgaggccttgcgcttaaggagccccttcgcctcgtgcttgagttgaggcct |
| | | ggcttgggcgctggggccgccgcgtgcgaatctggtggcaccttcgcgcctgtctcgctgctttcgataagtctc |
| | | tagccatttaaaatttttgatgacctgctgcgacgctttttttctggcaagatagtcttgtaaatgcgggccaaCatct |
| | | gcacactggtatttcggtttttggggccgcgggcggcgacggggcccgtgcgtcccagcgcacatgttcggcg |
| | | aggcggggcctgcgagcgcggccaccgagaatcggacgggggtagtctcaagctggccggcctgctctggt |
| | | gcctggcctcgcgccgccgtgtatcgccccgccctgggcggcaaggctggcccggtcggcaccagttgcgtg |
| | | agcggaaagatggccgcttcccggccctgctgcagggagctcaaaatggaggacgcggcgctcgggagagc |
| | | ggggggtgagtcacccacacaaaggaaaagggcctttccgtcctcagccgtcgcttcatgtgactccacgga |
| | | gtaccgggcgccgtccaggcacctcgattagttctcgagcttttggagtacgtcgtctttaggttggggggaggg |
| | | gttttatgcgatggagtttccccacactgagtgggtggagactgaagttaggccagcttggcacttgatgtaattct |
| | | ccttggaatttgccctttttgagtttggatcttggttcattctcaagcctcagacagtggttcaaagtttttttcttccattt |
| | | caggtgtcgtgacgctagcgctaccggactcaatctcgagctcaagcttcgaattctgcagtcgacggtaccgcg |
| | | ggcccgggatccaccggtcgccaccATGgtgAGCAAGGGCGAGGAGCTGTTCACC |
| | | GGGGTGGTGCCCATCCTGGTCGAGCTGGACGGCGACGTAAACGGCC |
| | | ACAAGTTCAGCGTGTCCGGCGAGGGCGAGGGCGATGCCACCTACG |
| | | GCAAGCTGACCCTGAAGTTCATCTGCACCACCGGCAAGCTGCCCGT |
| | | GCCCTGGCCCACCCTCGTGACCACCCTGACCTACGGCGTGCAGTGC |
| | | TTCAGCCGCTACCCCGACCACATGAAGCAGCACGACTTCTTCAAGT |
| | | CCGCCATGCCCGAAGGCTACGTCCAGGAGCGCACCATCTTCTTCAA |
| | | GGACGACGGCAACTACAAGACCCGCGCCGAGGTGAAGTTCGAGGG |
| | | CGACACCCTGGTGAACCGCATCGAGCTGAAGGGCATCGACTTCAAG |
| | | GAGGACGGCAACATCCTGGGGCACAAGCTGGAGTACAACTACAAC |
| | | AGCCACAACGTCTATATCATGGCCGACAAGCAGAAGAACGGCATC |
| | | AAGGTGAACTTCAAGATCCGCCACAACATCGAGGACGGCAGCGTG |
| | | CAGCTCGCCGACCACTACCAGCAGAACACCCCCATCGGCGACGGCC |
| | | CCGTGCTGCTGCCCGACAACCACTACCTGAGCACCCAGTCCGCCCT |
| | | GAGCAAAGACCCCAACGAGAAGCGCGATCACATGGTCCTGCTGGA |
| | | GTTCGTGACCGCCGCCGGGATCACTCTCGGCATGGACGAGCTGTAC |
| | | AAGTAAtagcggccgcgactctagatcataatcagccataccacatttgtagaggttttacttgctttaaaaaa |
| | | cctcccacacctccccctgaacctgaaacataaaatgaatgcaattgttgttgttaacttgtttattgcagcttataatg |
| | | gttacaaataaagcaatagcatcacaaatttcacaaataaagcatttttttcactgcattctagttgtggtttgtccaaa |
| | | ctcatcaatgtatcttaaggcgttgggaccaccttatattcccagggccggttaatgtggctctggttctgggtacttt |
| | | tatctgtcccctccaccccacagtggggccactagggacaggattggtgacagaaaagccccatccttaggcct |
| | | cctccttcctagtctcctgatattgggtctaacccccacctcctgttaggcagattccttatctggtgacacaccccc |
| | | atttcctggagccatctctctccttgccagaacctctaaggtttgcttacgatggagccagagaggatcctgggag |
| | | ggagagcttggcaggggggggagggaagggggggatgcgtgacctgcccggttctcagtggccaccctgc |
| | | gctaccctctcccagaacctgagctgctctgacgcggccgtctggtgcgtttcactgatcctggtgctgcagcttc |
| | | cttacacttcccaagaggagaagcagtttggaaaaacaaaatcagaataagttggtcctgagttctaactttggctc |
| | | ttcacctttctagtccccaatttatattgttcctccgtgcgtcagttttacctgtgagataaggccagtagccagcccc |
| | | gtcctggcagggctgtggtgaggaggggggtgtccgtgtggaaaactccctttgtgagaatggtgcgtcctagg |
| | | tgttcaccaggtcgtggccgcctctactccctttctctttctccatccttctttccttaaagagtccccagtgctatctg |
| | | ggacatattcctccgcccagagcagggtcccgcttccctaaggccctgctctgggcttctgggtttgagtccttgg |
| | | caagcccaggagaggcgctcaggcttccctgtcccccttcctcgtccaccatctcatgcccctggctctcctgcc |
| | | ccttccctacaggggttcctggctctgctcttcagactgagccccgttcccctgcatc |
| 300 | Template B eGFP | ccctgccctgccctctcctgaacctgagccagctcccatagctcagtctggtctatctgcctggccctggccattgt |
| | insertion | cactttgcgctgccctcctctcgcccccgagtgcccttgctgtgccgccggaactctgccctctaacgctgccgtc |
| | construct with | tctctcctgagtccggaccactttgagctctactggcttctgcgccgcctctggcccactgtttccccttcccaggc |
| | homology arms tc | aggtcctgctttctctgacctgcattctctcccctgggcctgtgccgctttctgtctgcagcttgtggcctgggtcac |
| | mouse AAVS1 | ctctacggctggcccagatccttccctgccgcctccttcaggttccgtcttcctccactccctcttccccttgctctct |
| | | gctgtgttgctgcccaaggatgctctttccggagcacttccttctcggcgctgcaccacgtgatgtcctctgagcg |
| | | gatcctccccgtgtctgggtcctctccgggcatctctcctccctcacccaaccccatgccgtcttcactcgctgggt |
| | | tcccttttccttctccttctggggcctgtgccatctctcgtttcttaggatggccttctccgacggatgtctcccttgcgt |
| | | cccgcctccccttcttgtaggcctgcatcatcaccgtttttctggacaaccccaaagtaccccgtctccctggcttta |
| | | gccacctctccatcctcttgctttctttgcctggacaccccgttctcctgtggattcgggtcacctctcactcctttcatt |
| | | tgggcagctcccctaccccccttacctctctagtctgtgctagctcttccagccccctgtcatggcatcttccaggg |
| | | gtccgagagctcagctagtcttcttcctccaacccgggcccctatgtccacttcaggacagcatgtttgctgcctcc |
| | | agggatcctgtgtccctagttattaatgagtaattcatacaaaaggactcgcccctgccttggggaatcccaggga |
| | | ccgtcgttaaactcccactaacgtagaacccagagatcgctgcgttcccgccccctcacccgcccgctctcgtca |
| | | tcactgaggtggagaagagcatgcgtgaggctccggtgcccgtcagtgggcagagcgcacatcgcccacagt |
| | | ccccgagaagttggggggaggggtcggcaattgaaccggtgcctagagaaggtggcgcggggtaaactggg |
| | | aaagtgatgtcgtgtactggctccgcctttttcccgaggggggggagaaccgtatataagtgcagtagtcgccgt |
| | | gaacgttctttttcgcaacgggtttgccgccagaacacaggtaagtgccgtgtgtggttcccgcgggcctggcct |
| | | ctttacgggttatggcccttgcgtgccttgaattacttccacgcccctggctgcagtacgtgattcttgatcccgagc |
| | | ttcgggttggaagtgggtgggagagttcgaggccttgcgcttaaggagccccttcgcctcgtgcttgagttgagg |
| | | cctggcttgggcgctggggccgccgcgtgcgaatctggtggcaccttcgcgcctgtctcgctgctttcgataagt |
| | | ctctagccatttaaaatttttgatgacctgctgcgacgctttttttctggcaagatagtcttgtaaatgcgggccaaCa |
| | | tctgcacactggtatttcggtttttggggccggggggcgacggggcccgtgcgtcccagcgcacatgttcgg |
| | | cgaggcggggcctgcgagcgcggccaccgagaatcggacgggggtagtctcaagctggccggcctgctctg |
| | | gtgcctggcctcgcgccgccgtgtatcgccccgccctgggcggcaaggctggcccggtcggcaccagttgcg |
| | | tgagcggaaagatggccgcttcccggccctgctgcagggagctcaaaatggaggacgcggcgctcgggaga |
| | | gcggggggtgagtcacccacacaaaggaaaagggcctttccgtcctcagccgtcgcttcatgtgactccacg |
| | | gagtaccgggcgccgtccaggcacctcgattagttctcgagcttttggagtacgtcgtctttaggttggggggag |
| | | gggttttatgcgatggagtttccccacactgagtgggtggagactgaagttaggccagcttggcacttgatgtaatt |
| | | ctccttggaatttgccctttttgagtttggatcttggttcattctcaagcctcagacagtggttcaaagtttttttcttccat |
| | | ttcaggtgtcgtgacgctagcgctaccggactcaatctcgagctcaagcttcgaattctgcagtcgacggtaccg |
| | | cgggcccgggatccaccggtcgccaccATGgtgAGCAAGGGCGAGGAGCTGTTCAC |
| | | CGGGGTGGTGCCCATCCTGGTCGAGCTGGACGGCGACGTAAACGGC |
| | | CACAAGTTCAGCGTGTCCGGCGAGGGCGAGGGCGATGCCACCTACG |
| | | GCAAGCTGACCCTGAAGTTCATCTGCACCACCGGCAAGCTGCCCGT |
| | | GCCCTGGCCCACCCTCGTGACCACCCTGACCTACGGCGTGCAGTGC |
| | | TTCAGCCGCTACCCCGACCACATGAAGCAGCACGACTTCTTCAAGT |
| | | CCGCCATGCCCGAAGGCTACGTCCAGGAGCGCACCATCTTCTTCAA |
| | | GGACGACGGCAACTACAAGACCCGCGCCGAGGTGAAGTTCGAGGG |
| | | CGACACCCTGGTGAACCGCATCGAGCTGAAGGGCATCGACTTCAAG |
| | | GAGGACGGCAACATCCTGGGGCACAAGCTGGAGTACAACTACAAC |
| | | AGCCACAACGTCTATATCATGGCCGACAAGCAGAAGAACGGCATC |
| | | AAGGTGAACTTCAAGATCCGCCACAACATCGAGGACGGCAGCGTG |
| | | CAGCTCGCCGACCACTACCAGCAGAACACCCCCATCGGCGACGGCC |
| | | CCGTGCTGCTGCCCGACAACCACTACCTGAGCACCCAGTCCGCCCT |
| | | GAGCAAAGACCCCAACGAGAAGCGCGATCACATGGTCCTGCTGGA |
| | | GTTCGTGACCGCCGCCGGGATCACTCTCGGCATGGACGAGCTGTAC |
| | | AAGTAAtagcggccgcgactctagatcataatcagccataccacatttgtagaggttttacttgctttaaaaaa |
| | | cctcccacacctccccctgaacctgaaacataaaatgaatgcaattgttgttgttaacttgtttattgcagcttataatg |
| | | gttacaaataaagcaatagcatcacaaatttcacaaataaagcatttttttcactgcattctagttgtggtttgtccaaa |
| | | ctcatcaatgtatcttaaggcgtagaaaagccccatccttaggcctcctccttcctagtctcctgatattgggtctaac |
| | | ccccacctcctgttaggcagattccttatctggtgacacacccccatttcctggagccatctctctccttgccagaac |
| | | ctctaaggtttgcttacgatggagccagagaggatcctgggagggagagcttggcagggggtgggagggaag |
| | | ggggggatgcgtgacctgcccggttctcagtggccaccctgcgctaccctctcccagaacctgagctgctctga |
| | | cgcggccgtctggtgcgtttcactgatcctggtgctgcagcttccttacacttcccaagaggagaagcagtttgga |
| | | aaaacaaaatcagaataagttggtcctgagttctaactttggctcttcacctttctagtccccaatttatattgttcctcc |
| | | gtgcgtcagttttacctgtgagataaggccagtagccagccccgtcctggcagggctgtggtgaggagggggg |
| | | tgtccgtgtggaaaactccctttgtgagaatggtgcgtcctaggtgttcaccaggtcgtggccgcctctactcccttt |
| | | ctctttctccatccttctttccttaaagagtccccagtgctatctgggacatattcctccgcccagagcagggtcccg |
| | | cttccctaaggccctgctctgggcttctgggtttgagtccttggcaagcccaggagaggcgctcaggcttccctgt |
| | | cccccttcctcgtccaccatctcatgcccctggctctcctgccccttccctacaggggttcctggctctgctcttcag |
| | | actgagccccgttcccctgcatccccgttcccctgcatcccccttcccctgcatcccccagaggccccaggccac |
| | | ctacttggcctggaccccacgagaggccaccccagccctgtctaccaggctgcct |
| 301 | Template C eGFP | taacgctgccgtctctctcctgagtccggaccactttgagctctactggcttctgcgccgcctctggcccactgtttc |
| | insertion | cccttcccaggcaggtcctgctttctctgacctgcattctctcccctgggcctgtgccgctttctgtctgcagcttgtg |
| | construct with | gcctgggtcacctctacggctggcccagatccttccctgccgcctccttcaggttccgtcttcctccactccctcttc |
| | homology arms to | cccttgctctctgctgtgttgctgcccaaggatgctctttccggagcacttccttctcggcgctgcaccacgtgatgt |
| | mouse AAVS1 | cctctgagcggatcctccccgtgtctgggtcctctccgggcatctctcctccctcacccaaccccatgccgtcttca |
| | | ctcgctgggttcccttttccttctccttctggggcctgtgccatctctcgtttcttaggatggccttctccgacggatgt |
| | | ctcccttgcgtcccgcctccccttcttgtaggcctgcatcatcaccgtttttctggacaaccccaaagtaccccgtct |
| | | ccctggctttagccacctctccatcctcttgctttctttgcctggacaccccgttctcctgtggattcgggtcacctctc |
| | | actcctttcatttgggcagctcccctaccccccttacctctctagtctgtgctagctcttccagccccctgtcatggca |
| | | tcttccaggggtccgagagctcagctagtcttcttcctccaacccgggcccctatgtccacttcaggacagcatgtt |
| | | tgctgcctccagggatcctgtgtccccgagctgggaccaccttatattcccagggccggttaatgtggctctggtt |
| | | ctgggtacttttatctgtcccctccaccccacagtggggccactagggacaggattggtgacagaaaagccccat |
| | | ccttaggcctcctcctagttattaatgagtaattcatacaaaaggactcgcccctgccttggggaatcccagggac |
| | | cgtcgttaaactcccactaacgtagaacccagagatcgctgcgttcccgccccctcacccgcccgctctcgtcat |
| | | cactgaggtggagaagagcatgcgtgaggctccggtgcccgtcagtgggcagagcgcacatcgcccacagtc |
| | | cccgagaagttggggggaggggtcggcaattgaaccggtgcctagagaaggtggcgcggggtaaactggga |
| | | aagtgatgtcgtgtactggctccgcctttttcccgaggggggggagaaccgtatataagtgcagtagtcgccgt |
| | | gaacgttctttttcgcaacgggtttgccgccagaacacaggtaagtgccgtgtgtggttcccgcgggcctggcct |
| | | ctttacgggttatggcccttgcgtgccttgaattacttccacgcccctggctgcagtacgtgattcttgatcccgagc |
| | | ttcgggttggaagtgggtgggagagttcgaggccttgcgcttaaggagccccttcgcctcgtgcttgagttgagg |
| | | cctggcttgggcgctggggccgccgcgtgcgaatctggtggcaccttcgcgcctgtctcgctgctttcgataagt |
| | | ctctagccatttaaaatttttgatgacctgctgcgacgctttttttctggcaagatagtcttgtaaatgcgggccaaCa |
| | | tctgcacactggtatttcggtttttggggccgcgggcggcgacggggcccgtgcgtcccagcgcacatgttcgg |
| | | cgaggggggcctgcgagcgcggccaccgagaatcggacgggggtagtctcaagctggccggcctgctctg |
| | | gtgcctggcctcgcgccgccgtgtatcgccccgccctgggggcaaggctggcccggtcggcaccagttgcg |
| | | tgagcggaaagatggccgcttcccggccctgctgcagggagctcaaaatggaggacgcggcgctcgggaga |
| | | gcggggggtgagtcacccacacaaaggaaaagggcctttccgtcctcagccgtcgcttcatgtgactccacg |
| | | gagtaccgggcgccgtccaggcacctcgattagttctcgagcttttggagtacgtcgtctttaggttggggggag |
| | | gggttttatgcgatggagtttccccacactgagtgggtggagactgaagttaggccagcttggcacttgatgtaatt |
| | | ctccttggaatttgccctttttgagtttggatcttggttcattctcaagcctcagacagtggttcaaagtttttttcttccat |
| | | ttcaggtgtcgtgacgctagcgctaccggactcaatctcgagctcaagcttcgaattctgcagtcgacggtaccg |
| | | cgggcccgggatccaccggtcgccaccATGgtgAGCAAGGGCGAGGAGCTGTTCAC |
| | | CGGGGTGGTGCCCATCCTGGTCGAGCTGGACGGCGACGTAAACGGC |
| | | CACAAGTTCAGCGTGTCCGGCGAGGGCGAGGGCGATGCCACCTACG |
| | | GCAAGCTGACCCTGAAGTTCATCTGCACCACCGGCAAGCTGCCCGT |
| | | GCCCTGGCCCACCCTCGTGACCACCCTGACCTACGGCGTGCAGTGC |
| | | TTCAGCCGCTACCCCGACCACATGAAGCAGCACGACTTCTTCAAGT |
| | | CCGCCATGCCCGAAGGCTACGTCCAGGAGCGCACCATCTTCTTCAA |
| | | GGACGACGGCAACTACAAGACCCGCGCCGAGGTGAAGTTCGAGGG |
| | | CGACACCCTGGTGAACCGCATCGAGCTGAAGGGCATCGACTTCAAG |
| | | GAGGACGGCAACATCCTGGGGCACAAGCTGGAGTACAACTACAAC |
| | | AGCCACAACGTCTATATCATGGCCGACAAGCAGAAGAACGGCATC |
| | | AAGGTGAACTTCAAGATCCGCCACAACATCGAGGACGGCAGCGTG |
| | | CAGCTCGCCGACCACTACCAGCAGAACACCCCCATCGGCGACGGCC |
| | | CCGTGCTGCTGCCCGACAACCACTACCTGAGCACCCAGTCCGCCCT |
| | | GAGCAAAGACCCCAACGAGAAGCGCGATCACATGGTCCTGCTGGA |
| | | GTTCGTGACCGCCGCCGGGATCACTCTCGGCATGGACGAGCTGTAC |
| | | AAGTAAtagcggccgcgactctagatcataatcagccataccacatttgtagaggttttacttgctttaaaaaa |
| | | cctcccacacctccccctgaacctgaaacataaaatgaatgcaattgttgttgttaacttgtttattgcagcttataatg |
| | | gttacaaataaagcaatagcatcacaaatttcacaaataaagcatttttttcactgcattctagttgtggtttgtccaaa |
| | | ctcatcaatgtatcttaaggcgttacgatggagccagagaggatcctgggagggagagcttggcagggggtgg |
| | | gagggaagggggggatgcgtgacctgcccggttctcagtggccaccctgcgctaccctctcccagaacctgag |
| | | ctgctctgacgcggccgtctggtgcgtttcactgatcctggtgctgcagcttccttacacttcccaagaggagaag |
| | | cagtttggaaaaacaaaatcagaataagttggtcctgagttctaactttggctcttcacctttctagtccccaatttata |
| | | ttgttcctccgtgcgtcagttttacctgtgagataaggccagtagccagccccgtcctggcagggctgtggtgagg |
| | | aggggggtgtccgtgtggaaaactccctttgtgagaatggtgcgtcctaggtgttcaccaggtcgtggccgcctc |
| | | tactccctttctctttctccatccttctttccttaaagagtccccagtgctatctgggacatattcctccgcccagagca |
| | | gggtcccgcttccctaaggccctgctctgggcttctgggtttgagtccttggcaagcccaggagaggcgctcag |
| | | gcttccctgtcccccttcctcgtccaccatctcatgcccctggctctcctgccccttccctacaggggttcctggctc |
| | | tgctcttcagactgagccccgttcccctgcatccccgttcccctgcatcccccttcccctgcatcccccagaggcc |
| | | ccaggccacctacttggcctggaccccacgagaggccaccccagccctgtctaccaggctgccttttgggtgga |
| | | ttctcctccaactgtggggtgactgcttggcaaactcactcttcggggtatcccaggaggcctggagcattggggt |
| | | gggctggggttcagagaggagggattcccttctcaggttacgtggccaagaagcaggggagc |
| 302 | Template D eGFP | gtttccccttcccaggcaggtcctgctttctctgacctgcattctctcccctgggcctgtgccgctttctgtctgcagc |
| | insertion | ttgtggcctgggtcacctctacggctggcccagatccttccctgccgcctccttcaggttccgtcttcctccactcc |
| | construct with | ctcttccccttgctctctgctgtgttgctgcccaaggatgctctttccggagcacttccttctcggcgctgcaccacg |
| | homology arms to | tgatgtcctctgagcggatcctccccgtgtctgggtcctctccgggcatctctcctccctcacccaaccccatgcc |
| | mouse AAVS1 | gtcttcactcgctgggttcccttttccttctccttctggggcctgtgccatctctcgtttcttaggatggccttctccgac |
| | | ggatgtctcccttgcgtcccgcctccccttcttgtaggcctgcatcatcaccgtttttctggacaaccccaaagtacc |
| | | ccgtctccctggctttagccacctctccatcctcttgctttctttgcctggacaccccgttctcctgtggattcgggtc |
| | | acctctcactcctttcatttgggcagctcccctaccccccttacctctctagtctgtgctagctcttccagccccctgt |
| | | catggcatcttccaggggtccgagagctcagctagtcttcttcctccaacccgggcccctatgtccacttcaggac |
| | | agcatgtttgctgcctccagggatcctgtgtccccgagctgggaccaccttatattcccagggccggttaatgtgg |
| | | ctctggttctgggtacttttatctgtcccctccaccccacagtggggccactagggacaggattggtgacagaaaa |
| | | gccccatccttaggcctcctccttcctagtctcctgatattgggtctaacccccacctcctgttaggcagattccttat |
| | | ctggtgacacaccccctagttattaatgagtaattcatacaaaaggactcgcccctgccttggggaatcccaggg |
| | | accgtcgttaaactcccactaacgtagaacccagagatcgctgcgttcccgccccctcacccgcccgcttcgtc |
| | | atcactgaggtggagaagagcatgcgtgaggctccggtgcccgtcagtgggcagagcgcacatcgcccacag |
| | | tccccgagaagttggggggaggggtcggcaattgaaccggtgcctagagaaggtggcgcggggtaaactgg |
| | | gaaagtgatgtcgtgtactggctccgcctttttcccgaggggggggagaaccgtatataagtgcagtagtcgcc |
| | | gtgaacgttctttttcgcaacgggtttgccgccagaacacaggtaagtgccgtgtgtggttcccgcgggcctggc |
| | | ctctttacgggttatggcccttgcgtgccttgaattacttccacgcccctggctgcagtacgtgattcttgatcccga |
| | | gcttcgggttggaagtgggtgggagagttcgaggccttgcgcttaaggagccccttcgcctcgtgcttgagttga |
| | | ggcctggcttgggcgctggggccgccgcgtgcgaatctggtggcaccttcgcgcctgtctcgctgctttcgataa |
| | | gtctctagccatttaaaatttttgatgacctgctgcgacgctttttttctggcaagatagtcttgtaaatgcgggccaa |
| | | Catctgcacactggtatttcggtttttggggccgcgggcggcgacggggcccgtgcgtcccagcgcacatgttc |
| | | ggcgaggcggggcctgcgagcgcggccaccgagaatcggacgggggtagtctcaagctggccggcctgct |
| | | ctggtgcctggcctcgcgccgccgtgtatcgccccgccctgggcggcaaggctggcccggtcggcaccagtt |
| | | gcgtgagcggaaagatggccgcttcccggccctgctgcagggagctcaaaatggaggacgcggcgctcggg |
| | | agagcggggggtgagtcacccacacaaaggaaaagggcctttccgtcctcagccgtcgcttcatgtgactcc |
| | | acggagtaccgggcgccgtccaggcacctcgattagttctcgagcttttggagtacgtcgtctttaggttggggg |
| | | gaggggttttatgcgatggagtttccccacactgagtgggtggagactgaagttaggccagcttggcacttgatgt |
| | | aattctccttggaatttgccctttttgagtttggatcttggttcattctcaagcctcagacagtggttcaaagtttttttctt |
| | | ccatttcaggtgtcgtgacgctagcgctaccggactcaatctcgagctcaagcttcgaattctgcagtcgacggta |
| | | ccgcgggcccgggatccaccggtcgccaccATGgtgAGCAAGGGCGAGGAGCTGTTC |
| | | ACCGGGGTGGTGCCCATCCTGGTCGAGCTGGACGGCGACGTAAACG |
| | | GCCACAAGTTCAGCGTGTCCGGCGAGGGCGAGGGCGATGCCACCTA |
| | | CGGCAAGCTGACCCTGAAGTTCATCTGCACCACCGGCAAGCTGCCC |
| | | GTGCCCTGGCCCACCCTCGTGACCACCCTGACCTACGGCGTGCAGT |
| | | GCTTCAGCCGCTACCCCGACCACATGAAGCAGCACGACTTCTTCAA |
| | | GTCCGCCATGCCCGAAGGCTACGTCCAGGAGCGCACCATCTTCTTC |
| | | AAGGACGACGGCAACTACAAGACCCGCGCCGAGGTGAAGTTCGAG |
| | | GGCGACACCCTGGTGAACCGCATCGAGCTGAAGGGCATCGACTTCA |
| | | AGGAGGACGGCAACATCCTGGGGCACAAGCTGGAGTACAACTACA |
| | | ACAGCCACAACGTCTATATCATGGCCGACAAGCAGAAGAACGGCA |
| | | TCAAGGTGAACTTCAAGATCCGCCACAACATCGAGGACGGCAGCGT |
| | | GCAGCTCGCCGACCACTACCAGCAGAACACCCCCATCGGCGACGGC |
| | | CCCGTGCTGCTGCCCGACAACCACTACCTGAGCACCCAGTCCGCCC |
| | | TGAGCAAAGACCCCAACGAGAAGCGCGATCACATGGTCCTGCTGG |
| | | AGTTCGTGACCGCCGCCGGGATCACTCTCGGCATGGACGAGCTGTA |
| | | CAAGTAAtagcggccgcgactctagatcataatcagccataccacatttgtagaggttttacttgctttaaaa |
| | | aacctcccacacctccccctgaacctgaaacataaaatgaatgcaattgttgttgttaacttgtttattgcagcttata |
| | | atggttacaaataaagcaatagcatcacaaatttcacaaataaagcatttttttcactgcattctagttgtggtttgtcc |
| | | aaactcatcaatgtatcttaaggcgtcggttctcagtggccaccctgcgctaccctctcccagaacctgagctgct |
| | | ctgacgcggccgtctggtgcgtttcactgatcctggtgctgcagcttccttacacttcccaagaggagaagcagtt |
| | | tggaaaaacaaaatcagaataagttggtcctgagttctaactttggctcttcacctttctagtccccaatttatattgttc |
| | | ctccgtgcgtcagttttacctgtgagataaggccagtagccagccccgtcctggcagggctgtggtgaggaggg |
| | | gggtgtccgtgtggaaaactccctttgtgagaatggtgcgtcctaggtgttcaccaggtcgtggccgcctctactc |
| | | cctttctctttctccatccttctttccttaaagagtccccagtgctatctgggacatattcctccgcccagagcagggt |
| | | cccgcttccctaaggccctgctctgggcttctgggtttgagtccttggcaagcccaggagaggcgctcaggcttc |
| | | cctgtcccccttcctcgtccaccatctcatgcccctggctctcctgccccttccctacaggggttcctggctctgctc |
| | | ttcagactgagccccgttcccctgcatccccgttcccctgcatcccccttcccctgcatcccccagaggccccag |
| | | gccacctacttggcctggaccccacgagaggccaccccagccctgtctaccaggctgccttttgggtggattctc |
| | | ctccaactgtggggtgactgcttggcaaactcactcttcggggtatcccaggaggcctggagcattggggtggg |
| | | ctggggttcagagaggagggattcccttctcaggttacgtggccaagaagcaggggagctgggtttgggtcag |
| | | gtctgggtgtggggtgaccagcttatgctgtttgcccaggacagcctagttttagcactgaaac |
| 303 | Template OG | actagtccgcgggcggccgcgctgccctcctctcgcccccgagtgcccttgctgtgccgccggaactctgccct |
| | eGFP insertion | ctaacgctgccgtctctctcctgagtccggaccactttgagctctactggcttctgcgccgcctctggcccactgttt |
| | construct with | ccccttcccaggcaggtcctgctttctctgacctgcattctctcccctgggcctgtgccgctttctgtctgcagcttgt |
| | homology arms to | ggcctgggtcacctctacggctggcccagatccttccctgccgcctccttcaggttccgtcttcctccactccctctt |
| | mouse AAVS1 | ccccttgctctctgctgtgttgctgcccaaggatgctctttccggagcacttccttctcggcgctgcaccacgtgat |
| | | gtcctctgagcggatcctccccgtgtctgggtcctctccgggcatctctcctccctcacccaaccccatgccgtctt |
| | | cactcgctgggttcccttttccttctccttctggggcctgtgccatctctcgtttcttaggatggccttctccgacggat |
| | | gtctcccttgcgtcccgcctccccttcttgtaggcctgcatcatcaccgtttttctggacaaccccaaagtaccccgt |
| | | ctccctggctttagccacctctccatcctcttgctttctttgcctggacaccccgttctcctgtggattcgggtcacctc |
| | | tcactcctttcatttgggcagctcccctaccccccttacctctctagtctgtgctagctcttccagccccctgtcatgg |
| | | catcttccaggggtccgagagctcagctagtcttcttcctccaacccgggcccctatgtccacttcaggacagcat |
| | | gtttgctgcctccagggatcctgtgtccccgagctgggaccaccttatattcccagggccggttaatgtggctctg |
| | | gttctgggtacttttatctgtcccctccaccccacagttagttattaatgagtaattcatacaaaaggactcgcccctg |
| | | ccttggggaatcccagggaccgtcgttaaactcccactaacgtagaacccagagatcgctgcgttcccgccccc |
| | | tcacccgcccgctctcgtcatcactgaggtggagaagagcatgcgtgaggctccggtgcccgtcagtgggcag |
| | | agcgcacatcgcccacagtccccgagaagttggggggaggggtcggcaattgaaccggtgcctagagaaggt |
| | | ggcgcggggtaaactgggaaagtgatgtcgtgtactggctccgcctttttcccgagggtgggggagaaccgtat |
| | | ataagtgcagtagtcgccgtgaacgttctttttcgcaacgggtttgccgccagaacacaggtaagtgccgtgtgtg |
| | | gttcccggggcctggcctctttacgggttatggcccttgcgtgccttgaattacttccacgcccctggctgcagta |
| | | cgtgattcttgatcccgagcttcgggttggaagtgggtgggagagttcgaggccttgcgcttaaggagccccttc |
| | | gcctcgtgcttgagttgaggcctggcttgggcgctggggccgccgcgtgcgaatctggtggcaccttcgcgcct |
| | | gtctcgctgctttcgataagtctctagccatttaaaatttttgatgacctgctgcgacgctttttttctggcaagatagtc |
| | | ttgtaaatgcgggccaacatctgcacactggtatttcggtttttggggccgcgggcggcgacggggcccgtgcg |
| | | tcccagcgcacatgttcggcgaggcggggcctgcgagcgcggccaccgagaatcggacgggggtagtctca |
| | | agctggccggcctgctctggtgcctggcctcgcgccgccgtgtatcgccccgccctgggcggcaaggctggc |
| | | ccggtcggcaccagttgcgtgagcggaaagatggccgcttcccggccctgctgcagggagctcaaaatggag |
| | | gacgcggcgctcgggagagcggggggtgagtcacccacacaaaggaaaagggcctttccgtcctcagccg |
| | | tcgcttcatgtgactccacggagtaccgggcgccgtccaggcacctcgattagttctcgagcttttggagtacgtc |
| | | gtctttaggttggggggaggggttttatgcgatggagtttccccacactgagtgggtggagactgaagttaggcc |
| | | agcttggcacttgatgtaattctccttggaatttgccctttttgagtttggatcttggttcattctcaagcctcagacagt |
| | | ggttcaaagtttttttcttccatttcaggtgtcgtgacaccggtcgccaccatggtgAGCAAGGGCGAG |
| | | GAGCTGTTCACCGGGGTGGTGCCCATCCTGGTCGAGCTGGACGGCG |
| | | ACGTAAACGGCCACAAGTTCAGCGTGTCCGGCGAGGGCGAGGGCG |
| | | ATGCCACCTACGGCAAGCTGACCCTGAAGTTCATCTGCACCACCGG |
| | | CAAGCTGCCCGTGCCCTGGCCCACCCTCGTGACCACCCTGACCTAC |
| | | GGCGTGCAGTGCTTCAGCCGCTACCCCGACCACATGAAGCAGCACG |
| | | ACTTCTTCAAGTCCGCCATGCCCGAAGGCTACGTCCAGGAGCGCAC |
| | | CATCTTCTTCAAGGACGACGGCAACTACAAGACCCGCGCCGAGGTG |
| | | AAGTTCGAGGGCGACACCCTGGTGAACCGCATCGAGCTGAAGGGC |
| | | ATCGACTTCAAGGAGGACGGCAACATCCTGGGGCACAAGCTGGAG |
| | | TACAACTACAACAGCCACAACGTCTATATCATGGCCGACAAGCAGA |
| | | AGAACGGCATCAAGGTGAACTTCAAGATCCGCCACAACATCGAGG |
| | | ACGGCAGCGTGCAGCTCGCCGACCACTACCAGCAGAACACCCCCAT |
| | | CGGCGACGGCCCCGTGCTGCTGCCCGACAACCACTACCTGAGCACC |
| | | CAGTCCGCCCTGAGCAAAGACCCCAACGAGAAGCGCGATCACATG |
| | | GTCCTGCTGGAGTTCGTGACCGCCGCCGGGATCACTCTCGGCATGG |
| | | ACGAGCTGTACAAGtaatagcggccgcgactctagatcataatcagccataccacatttgtagaggt |
| | | tttacttgctttaaaaaacctcccacacctccccctgaacctgaaacataaaatgaatgcaattgttgttgttaacttgt |
| | | ttattgcagcttataatggttacaaataaagcaatagcatcacaaatttcacaaataaagcatttttttcactgcattcta |
| | | gttgtggtttgtccaaactcatcaatgtatcttaaggcgttttcctggagccatctctctccttgccagaacctctaag |
| | | gtttgcttacgatggagccagagaggatcctgggagggagagcttggcaggggggggagggaaggggggg |
| | | atgcgtgacctgcccggttctcagtggccaccctgcgctaccctctcccagaacctgagctgctctgacgcggc |
| | | cgtctggtgcgtttcactgatcctggtgctgcagcttccttacacttcccaagaggagaagcagtttggaaaaaca |
| | | aaatcagaataagttggtcctgagttctaactttggctcttcacctttctagtccccaatttatattgttcctccgtgcgt |
| | | cagttttacctgtgagataaggccagtagccagccccgtcctggcagggctgtggtgaggaggggggtgtccgt |
| | | gtggaaaactccctttgtgagaatggtgcgtcctaggtgttcaccaggtcgtggccgcctctactccctttctctttct |
| | | ccatccttctttccttaaagagtccccagtgctatctgggacatattcctccgcccagagcagggtcccgcttccct |
| | | aaggccctgctctgggcttctgggtttgagtccttggcaagcccaggagaggcgctcaggcttccctgtccccct |
| | | tcctcgtccaccatctcatgcccctggctctcctgccccttccctacaggggttcctggctctgctcttcagactgag |
| | | ccccgttcccctgcatccccgttcccctgcatcccccttcccctgcatcccccagaggccccaggccacctactt |
| | | ggcctggaccccacgagaggccaccccagccctgtctaccaggctgccttttgggggattctcctccaactgtg |
| | | gggtgactgcttggcaaactcactcttcggggtatcccaggaggcctggagcattggggtgggctggggttcag |
| | | aggcggccgcccgcggactagt |
| 304 | Bidirectional | TTGGCCACTCCCTCTCTGCGCGCTCGCTCGCTCACTGAGGCCGGGCG |
| | NanoLuc | ACCAAAGGTCGCCCGACGCCCGGGCTTTGCCCGGGCGGCCTCAGTG |
| | insertion | AGCGAGCGAGCGCGCAGAGAGGGAGTGGCCAACTCCATCACTAGG |
| | Construct | GGTTCCTagatctctATAACTTCGTATAGCATACATTATACGAAGTTATA |
| | | TGTATGCtaggtcagtgaagagaagaacaaaaagcagcatattacagttagttgtcttcatcaatctttaaat |
| | | atgttgtgtggtttttctctccctgtttccacagtttttcttgatcatgaaaacgccaacaaaattctgaatcggccaaa |
| | | gaggtataattcaggtaaattggaagagtttgttcaagggaaccttgagagagaatgtatggaagaaaagtgtagt |
| | | tttgaagaagcaGTATTCACTTTGGAGGACTTTGTCGGTGACTGGAGGCAA |
| | | ACCGCTGGTTATAATCTCGACCAaGTACTGGAACAGGGCGGGGTAA |
| | | GTTCCCTCTTTCAGAATTTGGGTGTAAGCGTCACACCAATCCAGCGG |
| | | ATTGTGTTGTCTGGAGAGAACGGACTCAAAATTGACATCCATGTTA |
| | | TCATTCCATATGAAGGTCTCAGTGGAGACCAAATGGGGCAGATCGA |
| | | GAAGATTTTCAAGGTAGTTTACCCAGTCGACGATCACCACTTCAAA |
| | | GTCATtCTCCACTATGGCACACTTGTTATCGACGGAGTAACTCCTAA |
| | | TATGATTGATTACTTTGGTCGCCCGTATGAGGGCATCGCAGTGTTTG |
| | | ATGGCAAAAAGATCACCGTAACAGGAACGTTGTGGAATGGGAACA |
| | | AGATAATCGACGAGAGATTGATAAATCCAGACGGGTCACTCCTGTT |
| | | CAGGGTTACAATTAACGGCGTCACAGGATGGAGACTCTGTGAACGA |
| | | ATACTGGCCacaaatttttcactcctgaagcaggccggagacgtggaggaaaacccagggcccgtgA |
| | | GCAAGGGCGAGGAGCTGTTCACCGGGGTGGTGCCCATCCTGGTCGA |
| | | GCTGGACGGCGACGTAAACGGCCACAAGTTCAGCGTGTCCGGCGA |
| | | GGGCGAGGGCGATGCCACCTACGGCAAGCTGACCCTGAAGTTCATC |
| | | TGCACCACCGGCAAGCTGCCCGTGCCCTGGCCCACCCTCGTGACCA |
| | | CCCTGACCTACGGCGTGCAGTGCTTCAGCCGCTACCCCGACCACAT |
| | | GAAGCAGCACGACTTCTTCAAGTCCGCCATGCCCGAAGGCTACGTC |
| | | CAGGAGCGCACCATCTTCTTCAAGGACGACGGCAACTACAAGACCC |
| | | GCGCCGAGGTGAAGTTCGAGGGCGACACCCTGGTGAACCGCATCG |
| | | AGCTGAAGGGCATCGACTTCAAGGAGGACGGCAACATCCTGGGGC |
| | | ACAAGCTGGAGTACAACTACAACAGCCACAACGTCTATATCATGGC |
| | | CGACAAGCAGAAGAACGGCATCAAGGTGAACTTCAAGATCCGCCA |
| | | CAACATCGAGGACGGCAGCGTGCAGCTCGCCGACCACTACCAGCA |
| | | GAACACCCCCATCGGCGACGGCCCCGTGCTGCTGCCCGACAACCAC |
| | | TACCTGAGCACCCAGTCCGCCCTGAGCAAAGACCCCAACGAGAAGC |
| | | GCGATCACATGGTCCTGCTGGAGTTCGTGACCGCCGCCGGGATCAC |
| | | TCTCGGCATGGACGAGCTGTACAAGGGAGGAGGAAGCCCGAAGAA |
| | | GAAGAGAAAGGTCTAAcctCGACTGTGCCTTCTAGTTGCCAGCCATC |
| | | TGTTGTTTGCCCCTCCCCCGTGCCTTCCTTGACCCTGGAAGGTGCCA |
| | | CTCCCACTGTCCTTTCCTAATAAAATGAGGAAATTGCATCGCATTGT |
| | | CTGAGTAGGTGTCATTCTATTCTGGGGGGTGGGGTGGGGCAGGACA |
| | | GCAAGGGGGAGGATTGGGAAGACAATAGCAGGCATGCTGGGGATG |
| | | CGGTGGGCTCTATGGcttctgaggcggaaagaaccagctggggctctagggggtatccccAA |
| | | AAAACCTCCCACACCTCCCCCTGAACCTGAAACATAAAATGAATGC |
| | | AATTGTTGTTGTTAACTTGTTTATTGCAGCTTATAATGGTTACAAAT |
| | | AAAGCAATAGCATCACAAATTTCACAAATAAAGCATTTTTTTCACT |
| | | GCATTCTAGTTGTGGTTTGTCCAAACTCATCAATGTATCTTATCATG |
| | | TCTGTTACACCTTCCTCTTCTTCTTGGGGCTGCCGCCGCCCTTGTACA |
| | | GCTCGTCCATGCCCAGGGTGATGCCGGCGGCGGTCACGAACTCCAG |
| | | CAGCACCATGTGGTCCCTCTTCTCGTTGGGGTCCTTGCTCAGGGCGC |
| | | TCTGGGTGCTCAGGTAGTGGTTGTCGGGCAGCAGCACGGGGCCGTC |
| | | GCCGATGGGGGTGTTCTGCTGGTAGTGGTCGGCCAGCTGCACGCTG |
| | | CCGTCCTCGATGTTGTGCCTGATCTTGAAGTTCACCTTGATGCCGTT |
| | | CTTCTGCTTGTCGGCCATGATGTACACGTTGTGGCTGTTGTAGTTGT |
| | | ACTCCAGCTTGTGGCCCAGGATGTTGCCGTCCTCCTTGAAGTCGATG |
| | | CCCTTCAGCTCGATCCTGTTCACCAGGGTGTCGCCCTCGAACTTCAC |
| | | CTCGGCCCTGGTCTTGTAGTTGCCGTCGTCCTTGAAGAAGATGGTCC |
| | | TCTCCTGCACGTAGCCCTCGGGCATGGCGCTCTTGAAGAAGTCGTG |
| | | CTGCTTCATGTGGTCGGGGTACCTGCTGAAGCACTGCACGCCGTAG |
| | | GTCAGGGTGGTCACCAGGGTGGGCCAGGGCACGGGCAGCTTGCCG |
| | | GTGGTGCAGATGAACTTCAGGGTCAGCTTGCCGTAGGTGGCGTCGC |
| | | CCTCGCCCTCGCCGCTCACGCTGAACTTGTGGCCGTTCACGTCGCCG |
| | | TCCAGCTCCACCAGGATGGGCACCACGCCGGTGAACAGCTCCTCGC |
| | | CCTTGCTCACGGGGCCGGGGTTCTCCTCCACGTCGCCGGCCTGCTTC |
| | | AGCAGGCTGAAGTTGGTGGCCAGGATCCTCTCGCACAGCCTCCAGC |
| | | CGGTCACGCCGTTGATGGTCACCCTGAACAGCAGGCTGCCGTCGGG |
| | | GTTGATCAGCCTCTCGTCGATGATCTTGTTGCCGTTCCACAGGGTGC |
| | | CGGTCACGGTGATCTTCTTGCCGTCGAACACGGCGATGCCCTCGTA |
| | | GGGCCTGCCGAAGTAGTCGATCATGTTGGGGGTCACGCCGTCGATC |
| | | ACCAGGGTGCCGTAGTGCAGGATCACCTTGAAGTGGTGGTCGTCCA |
| | | CGGGGTACACCACCTTGAAAATCTTCTCGATCTGGCCCATCTGGTCG |
| | | CCGCTCAGGCCCTCGTAGGGGATGATCACGTGGATGTCGATCTTCA |
| | | GGCCGTTCTCGCCGCTCAGCACGATCCTCTGGATGGGGGTCACGCT |
| | | CACGCCCAGGTTCTGGAACAGGCTGCTCACGCCGCCCTGCTCCAGC |
| | | ACCTGGTCCAGGTTGTAGCCGGCGGTCTGCCTCCAGTCGCCCACGA |
| | | AGTCCTCCAGGGTGAACACGGCCTCCTCGAAGCTGCACTTCTCCTCC |
| | | ATGCACTCCCTCTCCAGGTTGCCCTGCACGAACTCCTCCAGCTTGCC |
| | | GCTGTTGTACCTCTTGGGCCTGTTCAGGATCTTGTTGGCGTTCTCGT |
| | | GGTCCAGGAAaactgtggaaacagggagagaaaaaccacacaacatatttaaagattgatgaagaca |
| | | actaactgtaatatgctgctttttgttcttctcttcactgacctaATGTATGCATAACTTCGTATAG |
| | | CATACATTATACGAAGTTATagagatctAGGAACCCCTAGTGATGGAGT |
| | | TGGCCACTCCCTCTCTGCGCGCTCGCTCGCTCACTGAGGCCGCCCGG |
| | | GCAAAGCCCGGGCGTCGGGCGACCTTTGGTCGCCCGGCCTCAGTGA |
| | | GCGAGCGAGCGCGCAGAGAGGGAGTGGCCAA |
| 305 | Open reading | AUGGAGGCCUCCCCCGCCUCCGGCCCCCGGCACCUGAUGGACCCC |
| | frame for Nme2 | CACAUCUUCACCUCCAACUUCAACAACGGCAUCGGCCGGCACAAG |
| | Cas9 | ACCUACCUGUGCUACGAGGUGGAGCGGCUGGACAACGGCACCUCC |
| | | GUGAAGAUGGACCAGCACCGGGGCUUCCUGCACAACCAGGCCAAG |
| | | AACCUGCUGUGCGGCUUCUACGGCCGGCACGCCGAGCUGCGGUUC |
| | | CUGGACCUGGUGCCCUCCCUGCAGCUGGACCCCGCCCAGAUCUAC |
| | | CGGGUGACCUGGUUCAUCUCCUGGUCCCCCUGCUUCUCCUGGGGC |
| | | UGCGCCGGCGAGGUGCGGGCCUUCCUGCAGGAGAACACCCACGUG |
| | | CGGCUGCGGAUCUUCGCCGCCCGGAUCUACGACUACGACCCCCUG |
| | | UACAAGGAGGCCCUGCAGAUGCUGCGGGACGCCGGCGCCCAGGUG |
| | | UCCAUCAUGACCUACGACGAGUUCAAGCACUGCUGGGACACCUUC |
| | | GUGGACCACCAGGGCUGCCCCUUCCAGCCCUGGGACGGCCUGGAC |
| | | GAGCACUCCCAGGCCCUGUCCGGCCGGCUGCGGGCCAUCCUGCAG |
| | | AACCAGGGCAACUCCGGCUCCGAGACCCCCGGCACCUCCGAGUCC |
| | | GCCACCCCCGAGUCCGACAAGAAGUACUCCAUCGGCCUGGCCAUC |
| | | GGCACCAACUCCGUGGGCUGGGCCGUGAUCACCGACGAGUACAAG |
| | | GUGCCCUCCAAGAAGUUCAAGGUGCUGGGCAACACCGACCGGCAC |
| | | UCCAUCAAGAAGAACCUGAUCGGCGCCCUGCUGUUCGACUCCGGC |
| | | GAGACCGCCGAGGCCACCCGGCUGAAGCGGACCGCCCGGCGGCGG |
| | | UACACCCGGCGGAAGAACCGGAUCUGCUACCUGCAGGAGAUCUUC |
| | | UCCAACGAGAUGGCCAAGGUGGACGACUCCUUCUUCCACCGGCUG |
| | | GAGGAGUCCUUCCUGGUGGAGGAGGACAAGAAGCACGAGCGGCA |
| | | CCCCAUCUUCGGCAACAUCGUGGACGAGGUGGCCUACCACGAGAA |
| | | GUACCCCACCAUCUACCACCUGCGGAAGAAGCUGGUGGACUCCAC |
| | | CGACAAGGCCGACCUGCGGCUGAUCUACCUGGCCCUGGCCCACAU |
| | | GAUCAAGUUCCGGGGCCACUUCCUGAUCGAGGGCGACCUGAACCC |
| | | CGACAACUCCGACGUGGACAAGCUGUUCAUCCAGCUGGUGCAGAC |
| | | CUACAACCAGCUGUUCGAGGAGAACCCCAUCAACGCCUCCGGCGU |
| | | GGACGCCAAGGCCAUCCUGUCCGCCCGGCUGUCCAAGUCCCGGCG |
| | | GCUGGAGAACCUGAUCGCCCAGCUGCCCGGCGAGAAGAAGAACGG |
| | | CCUGUUCGGCAACCUGAUCGCCCUGUCCCUGGGCCUGACCCCCAA |
| | | CUUCAAGUCCAACUUCGACCUGGCCGAGGACGCCAAGCUGCAGCU |
| | | GUCCAAGGACACCUACGACGACGACCUGGACAACCUGCUGGCCCA |
| | | GAUCGGCGACCAGUACGCCGACCUGUUCCUGGCCGCCAAGAACCU |
| | | GUCCGACGCCAUCCUGCUGUCCGACAUCCUGCGGGUGAACACCGA |
| | | GAUCACCAAGGCCCCCCUGUCCGCCUCCAUGAUCAAGCGGUACGA |
| | | CGAGCACCACCAGGACCUGACCCUGCUGAAGGCCCUGGUGCGGCA |
| | | GCAGCUGCCCGAGAAGUACAAGGAGAUCUUCUUCGACCAGUCCAA |
| | | GAACGGCUACGCCGGCUACAUCGACGGCGGCGCCUCCCAGGAGGA |
| | | GUUCUACAAGUUCAUCAAGCCCAUCCUGGAGAAGAUGGACGGCA |
| | | CCGAGGAGCUGCUGGUGAAGCUGAACCGGGAGGACCUGCUGCGG |
| | | AAGCAGCGGACCUUCGACAACGGCUCCAUCCCCCACCAGAUCCAC |
| | | CUGGGCGAGCUGCACGCCAUCCUGCGGCGGCAGGAGGACUUCUAC |
| | | CCCUUCCUGAAGGACAACCGGGAGAAGAUCGAGAAGAUCCUGACC |
| | | UUCCGGAUCCCCUACUACGUGGGCCCCCUGGCCCGGGGCAACUCC |
| | | CGGUUCGCCUGGAUGACCCGGAAGUCCGAGGAGACCAUCACCCCC |
| | | UGGAACUUCGAGGAGGUGGUGGACAAGGGCGCCUCCGCCCAGUCC |
| | | UUCAUCGAGCGGAUGACCAACUUCGACAAGAACCUGCCCAACGAG |
| | | AAGGUGCUGCCCAAGCACUCCCUGCUGUACGAGUACUUCACCGUG |
| | | UACAACGAGCUGACCAAGGUGAAGUACGUGACCGAGGGCAUGCG |
| | | GAAGCCCGCCUUCCUGUCCGGCGAGCAGAAGAAGGCCAUCGUGGA |
| | | CCUGCUGUUCAAGACCAACCGGAAGGUGACCGUGAAGCAGCUGA |
| | | AGGAGGACUACUUCAAGAAGAUCGAGUGCUUCGACUCCGUGGAG |
| | | AUCUCCGGCGUGGAGGACCGGUUCAACGCCUCCCUGGGCACCUAC |
| | | CACGACCUGCUGAAGAUCAUCAAGGACAAGGACUUCCUGGACAAC |
| | | GAGGAGAACGAGGACAUCCUGGAGGACAUCGUGCUGACCCUGAC |
| | | CCUGUUCGAGGACCGGGAGAUGAUCGAGGAGCGGCUGAAGACCU |
| | | ACGCCCACCUGUUCGACGACAAGGUGAUGAAGCAGCUGAAGCGGC |
| | | GGCGGUACACCGGCUGGGGCCGGCUGUCCCGGAAGCUGAUCAACG |
| | | GCAUCCGGGACAAGCAGUCCGGCAAGACCAUCCUGGACUUCCUGA |
| | | AGUCCGACGGCUUCGCCAACCGGAACUUCAUGCAGCUGAUCCACG |
| | | ACGACUCCCUGACCUUCAAGGAGGACAUCCAGAAGGCCCAGGUGU |
| | | CCGGCCAGGGCGACUCCCUGCACGAGCACAUCGCCAACCUGGCCG |
| | | GCUCCCCCGCCAUCAAGAAGGGCAUCCUGCAGACCGUGAAGGUGG |
| | | UGGACGAGCUGGUGAAGGUGAUGGGCCGGCACAAGCCCGAGAAC |
| | | AUCGUGAUCGAGAUGGCCCGGGAGAACCAGACCACCCAGAAGGGC |
| | | CAGAAGAACUCCCGGGAGCGGAUGAAGCGGAUCGAGGAGGGCAU |
| | | CAAGGAGCUGGGCUCCCAGAUCCUGAAGGAGCACCCCGUGGAGAA |
| | | CACCCAGCUGCAGAACGAGAAGCUGUACCUGUACUACCUGCAGAA |
| | | CGGCCGGGACAUGUACGUGGACCAGGAGCUGGACAUCAACCGGCU |
| | | GUCCGACUACGACGUGGACCACAUCGUGCCCCAGUCCUUCCUGAA |
| | | GGACGACUCCAUCGACAACAAGGUGCUGACCCGGUCCGACAAGAA |
| | | CCGGGGCAAGUCCGACAACGUGCCCUCCGAGGAGGUGGUGAAGA |
| | | AGAUGAAGAACUACUGGCGGCAGCUGCUGAACGCCAAGCUGAUC |
| | | ACCCAGCGGAAGUUCGACAACCUGACCAAGGCCGAGCGGGGCGGC |
| | | CUGUCCGAGCUGGACAAGGCCGGCUUCAUCAAGCGGCAGCUGGUG |
| | | GAGACCCGGCAGAUCACCAAGCACGUGGCCCAGAUCCUGGACUCC |
| | | CGGAUGAACACCAAGUACGACGAGAACGACAAGCUGAUCCGGGA |
| | | GGUGAAGGUGAUCACCCUGAAGUCCAAGCUGGUGUCCGACUUCC |
| | | GGAAGGACUUCCAGUUCUACAAGGUGCGGGAGAUCAACAACUAC |
| | | CACCACGCCCACGACGCCUACCUGAACGCCGUGGUGGGCACCGCC |
| | | CUGAUCAAGAAGUACCCCAAGCUGGAGUCCGAGUUCGUGUACGG |
| | | CGACUACAAGGUGUACGACGUGCGGAAGAUGAUCGCCAAGUCCG |
| | | AGCAGGAGAUCGGCAAGGCCACCGCCAAGUACUUCUUCUACUCCA |
| | | ACAUCAUGAACUUCUUCAAGACCGAGAUCACCCUGGCCAACGGCG |
| | | AGAUCCGGAAGCGGCCCCUGAUCGAGACCAACGGCGAGACCGGCG |
| | | AGAUCGUGUGGGACAAGGGCCGGGACUUCGCCACCGUGCGGAAG |
| | | GUGCUGUCCAUGCCCCAGGUGAACAUCGUGAAGAAGACCGAGGU |
| | | GCAGACCGGCGGCUUCUCCAAGGAGUCCAUCCUGCCCAAGCGGAA |
| | | CUCCGACAAGCUGAUCGCCCGGAAGAAGGACUGGGACCCCAAGAA |
| | | GUACGGCGGCUUCGACUCCCCCACCGUGGCCUACUCCGUGCUGGU |
| | | GGUGGCCAAGGUGGAGAAGGGCAAGUCCAAGAAGCUGAAGUCCG |
| | | UGAAGGAGCUGCUGGGCAUCACCAUCAUGGAGCGGUCCUCCUUCG |
| | | AGAAGAACCCCAUCGACUUCCUGGAGGCCAAGGGCUACAAGGAG |
| | | GUGAAGAAGGACCUGAUCAUCAAGCUGCCCAAGUACUCCCUGUUC |
| | | GAGCUGGAGAACGGCCGGAAGCGGAUGCUGGCCUCCGCCGGCGAG |
| | | CUGCAGAAGGGCAACGAGCUGGCCCUGCCCUCCAAGUACGUGAAC |
| | | UUCCUGUACCUGGCCUCCCACUACGAGAAGCUGAAGGGCUCCCCC |
| | | GAGGACAACGAGCAGAAGCAGCUGUUCGUGGAGCAGCACAAGCA |
| | | CUACCUGGACGAGAUCAUCGAGCAGAUCUCCGAGUUCUCCAAGCG |
| | | GGUGAUCCUGGCCGACGCCAACCUGGACAAGGUGCUGUCCGCCUA |
| | | CAACAAGCACCGGGACAAGCCCAUCCGGGAGCAGGCCGAGAACAU |
| | | CAUCCACCUGUUCACCCUGACCAACCUGGGCGCCCCCGCCGCCUU |
| | | CAAGUACUUCGACACCACCAUCGACCGGAAGCGGUACACCUCCAC |
| | | CAAGGAGGUGCUGGACGCCACCCUGAUCCACCAGUCCAUCACCGG |
| | | CCUGUACGAGACCCGGAUCGACCUGUCCCAGCUGGGCGGCGACGG |
| | | CGGCGGCUCCCCCAAGAAGAAGCGGAAGGUGUGA |
| 306 | Open reading | ATGgaggccTcccccgccTccggcccccggcaccTgaTggacccccacaTcTTcaccTccAAC |
| | frame for Sp. | TTCAACAACggcATCggccggCACAAGaccTACCTGTGCTACgaggTggagcg |
| | BC22n base | gCTGGACAACggcaccTccgTgAAGATGGACCAGCACcggggcTTCCTGCA |
| | editor | CAACCAGgccAAGAACCTGCTGTGCggcTTCTACggccggCACgccgagCTG |
| | | cggTTCCTGGACCTGgTgcccTccCTGCAGCTGGACcccgccCAGATCTACc |
| | | gggTgaccTGGTTCATCTccTGGTcccccTGCTTCTccTGGggcTGCgccggcgag |
| | | gTgcgggccTTCCTGCAGgagAACaccCACgTgcggCTGcggATCTTCgccgcccg |
| | | gATCTACGACTACGACcccCTGTACAAGgaggccCTGCAGATGCTGcggG |
| | | ACgccggcgccCAGgTgTccATCATGaccTACGACgagTTCAAGCACTGCTG |
| | | GGACaccTTCgTgGACCACCAGggcTGCcccTTCCAGcccTGGGACggcCTG |
| | | GACgagCACTccCAGgccCTGTccggccggCTGcgggccATCCTGCAGAACCA |
| | | GggcAACTccggcTccgagacccccggcaccTccgagTccgccacccccgagTccgacaagaagT |
| | | acTccaTcggccTggCcaTcggcaccaacTccgTgggcTgggccgTgaTcaccgacgagTacaag |
| | | gTgcccTccaagaagTTcaaggTgcTgggcaacaccgaccggcacTccaTcaagaagaaccTgaT |
| | | cggcgcccTgcTgTTcgacTccggcgagaccgccgaggccacccggcTgaagcggaccgcccggcg |
| | | gcggTacacccggcggaagaaccggaTcTgcTaccTgcaggagaTcTTcTccaacgagaTggcca |
| | | aggTggacgacTccTTcTTccaccggcTggaggagTccTTccTggTggaggaggacaagaagca |
| | | cgagcggcaccccaTcTTcggcaacaTcgTggacgaggTggccTaccacgagaagTaccccaccaT |
| | | cTaccaccTgcggaagaagcTggTggacTccaccgacaaggccgaccTgcggcTgaTcTaccTgg |
| | | cccTggcccacaTgaTcaagTTccggggccacTTccTgaTcgagggcgaccTgaaccccgacaac |
| | | TccgacgTggacaagcTgTTcaTccagcTggTgcagaccTacaaccagcTgTTcgaggagaaccc |
| | | caTcaacgccTccggcgTggacgccaaggccaTccTgTccgcccggcTgTccaagTcccggggc |
| | | TggagaaccTgaTcgcccagcTgcccggcgagaagaagaacggccTgTTcggcaaccTgaTcgcc |
| | | cTgTcccTgggccTgacccccaacTTcaagTccaacTTcgaccTggccgaggacgccaagcTgca |
| | | gcTgTccaaggacaccTacgacgacgaccTggacaaccTgcTggcccagaTcggcgaccagTacgc |
| | | cgaccTgTTccTggccgccaagaaccTgTccgacgccaTccTgcTgTccgacaTccTgcgggTg |
| | | aacaccgagaTcaccaaggccccccTgTccgccTccaTgaTcaagcggTacgacgagcaccaccagg |
| | | accTgacccTgcTgaaggcccTggTgcggcagcagcTgcccgagaagTacaaggagaTcTTcTT |
| | | cgaccagTccaagaacggcTacgccggcTacaTcgacggcggcgccTcccaggaggagTTcTacaa |
| | | gTTcaTcaagcccaTccTggagaagaTggacggcaccgaggagcTgcTggTgaagcTgaaccggg |
| | | aggaccTgcTgcggaagcagcggaccTTcgacaacggcTccaTcccccaccagaTccaccTgggg |
| | | agcTgcacgccaTccTgcggcggcaggaggacTTcTaccccTTccTgaaggacaaccgggagaag |
| | | aTcgagaagaTccTgaccTTccggaTccccTacTacgTgggcccccTggcccggggcaacTcccg |
| | | gTTcgccTggaTgacccggaagTccgaggagaccaTcacccccTggaacTTcgaggaggTggTg |
| | | gacaagggcgccTccgcccagTccTTcaTcgagcggaTgaccaacTTcgacaagaaccTgcccaac |
| | | gagaaggTgcTgcccaagcacTcccTgcTgTacgagTacTTcaccgTgTacaacgagcTgaccaa |
| | | ggTgaagTacgTgaccgagggcaTgcggaagcccgccTTccTgTccggcgagcagaagaaggcca |
| | | TcgTggaccTgcTgTTcaagaccaaccggaaggTgaccgTgaagcagcTgaaggaggacTacTT |
| | | caagaagaTcgagTgcTTcgacTccgTggagaTcTccggcgTggaggaccggTTcaacgccTcc |
| | | cTgggcaccTaccacgaccTgcTgaagaTcaTcaaggacaaggacTTccTggacaacgaggagaac |
| | | gaggacaTccTggaggacaTcgTgcTgacccTgacccTgTTcgaggaccgggagaTgaTcgagg |
| | | agcggcTgaagaccTacgcccaccTgTTcgacgacaaggTgaTgaagcagcTgaagcggcggcgg |
| | | TacaccggcTggggccggcTgTcccggaagcTgaTcaacggcaTccgggacaagcagTccggcaa |
| | | gaccaTccTggacTTccTgaagTccgacggcTTcgccaaccggaacTTcaTgcagcTgaTccac |
| | | gacgacTcccTgaccTTcaaggaggacaTccagaaggcccaggTgTccggccagggcgacTcccT |
| | | gcacgagcacaTcgccaaccTggccggcTcccccgccaTcaagaagggcaTccTgcagaccgTgaa |
| | | ggTggTggacgagcTggTgaaggTgaTgggccggcacaagcccgagaacaTcgTgaTcgagaTg |
| | | gcccgggagaaccagaccacccagaagggccagaagaacTcccgggagcggaTgaagcggaTcgagg |
| | | agggcaTcaaggagcTgggcTcccagaTccTgaaggagcaccccgTggagaacacccagcTgcaga |
| | | acgagaagcTgTaccTgTacTaccTgcagaacggccgggacaTgTacgTggaccaggagcTggac |
| | | aTcaaccggcTgTccgacTacgacgTggaccacaTcgTgccccagTccTTccTgaaggacgacTc |
| | | caTcgacaacaaggTgcTgacccggTccgacaagaaccggggcaagTccgacaacgTgcccTccga |
| | | ggaggTggTgaagaagaTgaagaacTacTggcggcagcTgcTgaacgccaagcTgaTcacccagc |
| | | ggaagTTcgacaaccTgaccaaggccgagcggggggccTgTccgagcTggacaaggccggcTTc |
| | | aTcaagcggcagcTggTggagacccggcagaTcaccaagcacgTggcccagaTccTggacTcccgg |
| | | aTgaacaccaagTacgacgagaacgacaagcTgaTccgggaggTgaaggTgaTcacccTgaagTc |
| | | caagcTggTgTccgacTTccggaaggacTTccagTTcTacaaggTgcgggagaTcaacaacTac |
| | | caccacgcccacgacgccTaccTgaacgccgTggTgggcaccgcccTgaTcaagaagTaccccaagc |
| | | TggagTccgagTTcgTgTacggcgacTacaaggTgTacgacgTgcggaagaTgaTcgccaagTc |
| | | cgagcaggagaTcggcaaggccaccgccaagTacTTcTTcTacTccaacaTcaTgaacTTcTTc |
| | | aagaccgagaTcacccTggccaacggcgagaTccggaagcggccccTgaTcgagaccaacggcgaga |
| | | ccggcgagaTcgTgTgggacaagggccgggacTTcgccaccgTgcggaaggTgcTgTccaTgcc |
| | | ccaggTgaacaTcgTgaagaagaccgaggTgcagaccggggcTTcTccaaggagTccaTccTgc |
| | | ccaagcggaacTccgacaagcTgaTcgcccggaagaaggacTgggaccccaagaagTacggcggcT |
| | | TcgacTcccccaccgTggccTacTccgTgcTggTggTggccaaggTggagaagggcaagTccaa |
| | | gaagcTgaagTccgTgaaggagcTgcTgggcaTcaccaTcaTggagcggTccTccTTcgagaag |
| | | aaccccaTcgacTTccTggaggccaagggcTacaaggaggTgaagaaggaccTgaTcaTcaagcT |
| | | gcccaagTacTcccTgTTcgagcTggagaacggccggaagcggaTgcTggccTccgccggcgagc |
| | | TgcagaagggcaacgagcTggcccTgcccTccaagTacgTgaacTTccTgTaccTggccTcccac |
| | | TacgagaagcTgaagggcTcccccgaggacaacgagcagaagcagcTgTTcgTggagcagcacaag |
| | | cacTaccTggacgagaTcaTcgagcagaTcTccgagTTcTccaagcgggTgaTccTggccgacg |
| | | ccaaccTggacaaggTgcTgTccgccTacaacaagcaccgggacaagcccaTccgggagcaggccga |
| | | gaacaTcaTccaccTgTTcacccTgaccaaccTgggcgcccccgccgccTTcaagTacTTcgaca |
| | | ccaccaTcgaccggaagcggTacaccTccaccaaggaggTgcTggacgccacccTgaTccaccagT |
| | | ccaTcaccggccTgTacgagacccggaTcgaccTgTcccagcTgggcggcgacggcggcggcTcc |
| | | cccaagaagaagcggaaggTgTgA |
| 307 | Open reading | aTggacaagaagTacTccaTcggccTggacaTcggcaccaacTccgTgggcTgggccgTgaTcac |
| | frame for Sp. | cgacgagTacaaggTgcccTccaagaagTTcaaggTgcTgggcaacaccgaccggcacTccaTcaa |
| | Cas9 | gaagaaccTgaTcggcgcccTgcTgTTcgacTccggcgagaccgccgaggccacccggcTgaagc |
| | | ggaccgcccggcggcggTacacccggcggaagaaccggaTcTgcTaccTgcaggagaTcTTcTcc |
| | | aacgagaTggccaaggTggacgacTccTTcTTccaccggcTggaggagTccTTccTggTggagg |
| | | aggacaagaagcacgagcggcaccccaTcTTcggcaacaTcgTggacgaggTggccTaccacgaga |
| | | agTaccccaccaTcTaccaccTgcggaagaagcTggTggacTccaccgacaaggccgaccTgcggc |
| | | TgaTcTaccTggcccTggcccacaTgaTcaagTTccggggccacTTccTgaTcgagggcgaccT |
| | | gaaccccgacaacTccgacgTggacaagcTgTTcaTccagcTggTgcagaccTacaaccagcTgT |
| | | TcgaggagaaccccaTcaacgccTccggcgTggacgccaaggccaTccTgTccgcccggcTgTcca |
| | | agTcccggcggcTggagaaccTgaTcgcccagcTgcccggcgagaagaagaacggccTgTTcggc |
| | | aaccTgaTcgcccTgTcccTgggccTgacccccaacTTcaagTccaacTTcgaccTggccgagga |
| | | cgccaagcTgcagcTgTccaaggacaccTacgacgacgaccTggacaaccTgcTggcccagaTcgg |
| | | cgaccagTacgccgaccTgTTccTggccgccaagaaccTgTccgacgccaTccTgcTgTccgaca |
| | | TccTgcgggTgaacaccgagaTcaccaaggccccccTgTccgccTccaTgaTcaagcggTacgac |
| | | gagcaccaccaggaccTgacccTgcTgaaggcccTggTgcggcagcagcTgcccgagaagTacaag |
| | | gagaTcTTcTTcgaccagTccaagaacggcTacgccggcTacaTcgacggcggcgccTcccagga |
| | | ggagTTcTacaagTTcaTcaagcccaTccTggagaagaTggacggcaccgaggagcTgcTggTg |
| | | aagcTgaaccgggaggaccTgcTgcggaagcagcggaccTTcgacaacggcTccaTcccccaccag |
| | | aTccaccTgggcgagcTgcacgccaTccTgcggcggcaggaggacTTcTaccccTTccTgaagga |
| | | caaccgggagaagaTcgagaagaTccTgaccTTccggaTccccTacTacgTgggcccccTggccc |
| | | ggggcaacTcccggTTcgccTggaTgacccggaagTccgaggagaccaTcacccccTggaacTTc |
| | | gaggaggTggTggacaagggcgccTccgcccagTccTTcaTcgagcggaTgaccaacTTcgacaa |
| | | gaaccTgcccaacgagaaggTgcTgcccaagcacTcccTgcTgTacgagTacTTcaccgTgTaca |
| | | acgagcTgaccaaggTgaagTacgTgaccgagggcaTgcggaagcccgccTTccTgTccggcgag |
| | | cagaagaaggccaTcgTggaccTgcTgTTcaagaccaaccggaaggTgaccgTgaagcagcTgaa |
| | | ggaggacTacTTcaagaagaTcgagTgcTTcgacTccgTggagaTcTccggcgTggaggaccgg |
| | | TTcaacgccTcccTgggcaccTaccacgaccTgcTgaagaTcaTcaaggacaaggacTTccTgga |
| | | caacgaggagaacgaggacaTccTggaggacaTcgTgcTgacccTgacccTgTTcgaggaccggg |
| | | agaTgaTcgaggagcggcTgaagaccTacgcccaccTgTTcgacgacaaggTgaTgaagcagcTg |
| | | aagcggcggcggTacaccggcTggggccggcTgTcccggaagcTgaTcaacggcaTccgggacaa |
| | | gcagTccggcaagaccaTccTggacTTccTgaagTccgacggcTTcgccaaccggaacTTcaTgc |
| | | agcTgaTccacgacgacTcccTgaccTTcaaggaggacaTccagaaggcccaggTgTccggccag |
| | | ggcgacTcccTgcacgagcacaTcgccaaccTggccggcTcccccgccaTcaagaagggcaTccTg |
| | | cagaccgTgaaggTggTggacgagcTggTgaaggTgaTgggccggcacaagcccgagaacaTcgT |
| | | gaTcgagaTggcccgggagaaccagaccacccagaagggccagaagaacTcccgggagcggaTgaag |
| | | cggaTcgaggagggcaTcaaggagcTgggcTcccagaTccTgaaggagcaccccgTggagaacacc |
| | | cagcTgcagaacgagaagcTgTaccTgTacTaccTgcagaacggccgggacaTgTacgTggacca |
| | | ggagcTggacaTcaaccggcTgTccgacTacgacgTggaccacaTcgTgccccagTccTTccTga |
| | | aggacgacTccaTcgacaacaaggTgcTgacccggTccgacaagaaccggggcaagTccgacaacg |
| | | TgcccTccgaggaggTggTgaagaagaTgaagaacTacTggcggcagcTgcTgaacgccaagcTg |
| | | aTcacccagcggaagTTcgacaaccTgaccaaggccgagcggggcggccTgTccgagcTggacaag |
| | | gccggcTTcaTcaagcggcagcTggTggagacccggcagaTcaccaagcacgTggcccagaTccT |
| | | ggacTcccggaTgaacaccaagTacgacgagaacgacaagcTgaTccgggaggTgaaggTgaTcac |
| | | ccTgaagTccaagcTggTgTccgacTTccggaaggacTTccagTTcTacaaggTgcgggagaTc |
| | | aacaac TaccaccacgcccacgacgccTaccTgaacgccgTggTgggcaccgcccTgaTcaagaag |
| | | TaccccaagcTggagTccgagTTcgTgTacggcgacTacaaggTgTacgacgTgcggaagaTga |
| | | TcgccaagTccgagcaggagaTcggcaaggccaccgccaagTacTTcTTcTacTccaacaTcaTg |
| | | aacTTcTTcaagaccgagaTcacccTggccaacggcgagaTccggaagcggccccTgaTcgagacc |
| | | aacggcgagaccggcgagaTcgTgTgggacaagggccgggacTTcgccaccgTgcggaaggTgcT |
| | | gTccaTgccccaggTgaacaTcgTgaagaagaccgaggTgcagaccggcggcTTcTccaaggagT |
| | | ccaTccTgcccaagcggaacTccgacaagcTgaTcgcccggaagaaggacTgggaccccaagaagT |
| | | acggcggcTTcgacTcccccaccgTggccTacTccgTgcTggTggTggccaaggTggagaaggg |
| | | caagTccaagaagcTgaagTccgTgaaggagcTgcTgggcaTcaccaTcaTggagcggTccTcc |
| | | TTcgagaagaaccccaTcgacTTccTggaggccaagggcTacaaggaggTgaagaaggaccTgaT |
| | | caTcaagcTgcccaagTacTcccTgTTcgagcTggagaacggccggaagcggaTgcTggccTccg |
| | | ccggcgagcTgcagaagggcaacgagcTggcccTgcccTccaagTacgTgaacTTccTgTaccTg |
| | | gccTcccacTacgagaagcTgaagggcTcccccgaggacaacgagcagaagcagcTgTTcgTggag |
| | | cagcacaagcacTaccTggacgagaTcaTcgagcagaTcTccgagTTcTccaagcgggTgaTccT |
| | | ggccgacgccaaccTggacaaggTgcTgTccgccTacaacaagcaccgggacaagcccaTccggga |
| | | gcaggccgagaacaTcaTccaccTgTTcacccTgaccaaccTgggcgcccccgccgccTTcaagTa |
| | | cTTcgacaccaccaTcgaccggaagcggTacaccTccaccaaggaggTgcTggacgccacccTgaT |
| | | ccaccagTccaTcaccggccTgTacgagacccggaTcgaccTgTcccagcTgggcggcgacggcgg |
| | | cggcTcccccaagaagaagcggaaggTgTga |
| 308 | Open reading | AUGGACGGCUCCGGCGGCGGCUCCCCCAAGAAGAAGCGGAAGGUG |
| | frame for Nme2 | GAGGACAAGCGGCCCGCCGCCACCAAGAAGGCCGGCCAGGCCAAG |
| | BC22n base | AAGAAGAAGGGCGGCUCCGGCGGCGGCGAGGCCUCCCCCGCCUCC |
| | editor | GGCCCCCGGCACCUGAUGGACCCCCACAUCUUCACCUCCAACUUC |
| | | AACAACGGCAUCGGCCGGCACAAGACCUACCUGUGCUACGAGGUG |
| | | GAGCGGCUGGACAACGGCACCUCCGUGAAGAUGGACCAGCACCGG |
| | | GGCUUCCUGCACAACCAGGCCAAGAACCUGCUGUGCGGCUUCUAC |
| | | GGCCGGCACGCCGAGCUGCGGUUCCUGGACCUGGUGCCCUCCCUG |
| | | CAGCUGGACCCCGCCCAGAUCUACCGGGUGACCUGGUUCAUCUCC |
| | | UGGUCCCCCUGCUUCUCCUGGGGCUGCGCCGGCGAGGUGCGGGCC |
| | | UUCCUGCAGGAGAACACCCACGUGCGGCUGCGGAUCUUCGCCGCC |
| | | CGGAUCUACGACUACGACCCCCUGUACAAGGAGGCCCUGCAGAUG |
| | | CUGCGGGACGCCGGCGCCCAGGUGUCCAUCAUGACCUACGACGAG |
| | | UUCAAGCACUGCUGGGACACCUUCGUGGACCACCAGGGCUGCCCC |
| | | UUCCAGCCCUGGGACGGCCUGGACGAGCACUCCCAGGCCCUGUCC |
| | | GGCCGGCUGCGGGCCAUCCUGCAGAACCAGGGCAACUCCGGCUCC |
| | | GAGACCCCCGGCACCUCCGAGUCCGCCACCCCCGAGUCCGCAGCG |
| | | UUCAAACCAAAUcccaucaacuacauccugggccuggccaucggcaucgccuccgugggcu |
| | | gggccaugguggagaucgacgaggaggagaaccccauccggcugaucgaccugggcgugcgggugu |
| | | ucgagcgggccgaggugcccaagaccggcgacucccuggccauggcccggggcuggcccgguccgu |
| | | gcggcggcugacccgggggggcccaccggcugcugcgggcccggggcugcugaagcgggaggg |
| | | cgugcugcaggccgccgacuucgacgagaacggccugaucaagucccugcccaacacccccuggcagc |
| | | ugcgggccgccgcccuggaccggaagcugaccccccuggagugguccgccgugcugcugcaccugau |
| | | caagcaccggggcuaccugucccagcggaagaacgagggcgagaccgccgacaaggagcugggcgccc |
| | | ugcugaagggcguggccaacaacgcccacgcccugcagaccggcgacuuccggacccccgccgagcug |
| | | gcccugaacaaguucgagaaggaguccggccacauccggaaccagcgggggacuacucccacaccuu |
| | | cucccggaaggaccugcaggccgagcugauccugcuguucgagaagcagaaggaguucggcaacccc |
| | | cacguguccggcggccugaaggagggcaucgagacccugcugaugacccagcggcccgcccuguccg |
| | | gcgacgccgugcagaagaugcugggccacugcaccuucgagcccgccgagcccaaggccgccaagaac |
| | | accuacaccgccgagcgguucaucuggcugaccaagcugaacaaccugcggauccuggagcagggcu |
| | | ccgagcggccccugaccgacaccgagcgggccacccugauggacgagcccuaccggaaguccaagcug |
| | | accuacgcccaggcccggaagcugcugggccuggaggacaccgccuucuucaagggccugcgguacg |
| | | gcaaggacaacgccgaggccuccacccugauggagaugaaggccuaccacgccaucucccgggcccug |
| | | gagaaggagggccugaaggacaagaaguccccccugaaccuguccuccgagcugcaggacgagaucg |
| | | gcaccgccuucucccuguucaagaccgacgaggacaucaccggccggcugaaggaccgggugcagccc |
| | | gagauccuggaggcccugcugaagcacaucuccuucgacaaguucgugcagaucucccugaaggccc |
| | | ugcggcggaucgugccccugauggagcagggcaagcgguacgacgaggccugcgccgagaucuacgg |
| | | cgaccacuacggcaagaagaacaccgaggagaagaucuaccugccccccauccccgccgacgagaucc |
| | | ggaaccccguggugcugcgggcccugucccaggcccggaaggugaucaacggcguggugcggggu |
| | | acggcucccccgcccggauccacaucgagaccgcccgggaggugggcaaguccuucaaggaccggaag |
| | | gagaucgagaagcggcaggaggagaaccggaaggaccgggagaaggccgccgccaaguuccgggagu |
| | | acuuccccaacuucgugggcgagcccaaguccaaggacauccugaagcugcggcuguacgagcagca |
| | | gcacggcaagugccuguacuccggcaaggagaucaaccuggugcggcugaacgagaagggcuacgug |
| | | gagaucgaccacgcccugcccuucucccggaccugggacgacuccuucaacaacaaggugcuggugc |
| | | ugggcuccgagaaccagaacaagggcaaccagacccccuacgaguacuucaacggcaaggacaacucc |
| | | cgggaguggcaggaguucaaggcccggguggagaccucccgguucccccgguccaagaagcagcgga |
| | | uccugcugcagaaguucgacgaggacggcuucaaggagugcaaccugaacgacacccgguacgugaa |
| | | ccgcuuccugugccaguucguggccgaccacauccugcugaccggcaagggcaagggggguguuc |
| | | gccuccaacggccagaucaccaaccugcugggggcuucuggggccugcggaagguggggccgaga |
| | | acgaccggcaccacgcccuggacgccguggugguggccugcuccaccguggccaugcagcagaagau |
| | | cacccgguucgugcgguacaaggagaugaacgccuucgacggcaagaccaucgacaaggagaccggca |
| | | aggugcugcaccagaagacccacuucccccagcccugggaguuuucgcccaggaggugaugauccg |
| | | gguguucggcaagcccgacggcaagcccgaguucgaggaggccgacacccccgagaagcugcggacc |
| | | cugcuggccgagaagcuguccucccggcccgaggccgugcacgaguacgugaccccccuguucgugu |
| | | cccgggcccccaaccggaagauguccggcgcccacaaggacacccugcgguccgccaagcgguucgug |
| | | aagcacaacgagaagaucuccgugaagcggguguggcugaccgagaucaagcuggccgaccuggaga |
| | | acauggugaacuacaagaacggccgggagaucgagcuguacgaggcccugaaggcccggcuggaggc |
| | | cuacggcggcaacgccaagcaggccuucgaccccaaggacaaccccuucuacaagaagggcggccagc |
| | | uggugaaggccgugggguggagaagacccaggaguccggcgugcugcugaacaagaagaacgccua |
| | | caccaucgccgacaacggcgacauggugcggguggacguguucugcaagguggacaagaagggcaag |
| | | aaccaguacuucaucgugcccaucuacgccuggcagguggcgagaacauccugcccgacaucgacug |
| | | caagggcuaccggaucgacgacuccuacaccuucugcuucucccugcacaaguacgaccugaucgccu |
| | | uccagaaggacgagaaguccaagguggaguucgccuacuacaucaacugcgacuccuccaacggccgg |
| | | uucuaccuggccuggcacgacaagggcuccaaggagcagcaguuccggaucuccacccagaaccugg |
| | | ugcugauccagaaguaccaggugaacgagcugggcaaggagauccggcccugccggcugaagaagcg |
| | | gccccccgugcgguag |
| 309 | Open reading | AUGACCAACCUGUCCGACAUCAUCGAGAAGGAGACCGGCAAGCAG |
| | frame for uracil | CUGGUGAUCCAGGAGUCCAUCCUGAUGCUGCCCGAGGAGGUGGA |
| | glycosylase | GGAGGUGAUCGGCAACAAGCCCGAGUCCGACAUCCUGGUGCACAC |
| | inhibitor (UGI) | CGCCUACGACGAGUCCACCGACGAGAACGUGAUGCUGCUGACCUC |
| | | CGACGCCCCCGAGUACAAGCCCUGGGCCCUGGUGAUCCAGGACUC |
| | | CAACGGCGAGAACAAGAUCAAGAUGCUGUCCGGCGGCUCCAAGCG |
| | | GACCGCCGACGGCUCCGAGUUCGAGUCCCCCAAGAAGAAGCGGAA |
| | | GGUGGAGUGAUAG |
| 310 | Open reading | AUGGACGGCUCCGGCGGCGGCUCCCCCAAGAAGAAGCGGAAGGUG |
| | frame encoding | GAGGACAAGCGGCCCGCCGCCACCAAGAAGGCCGGCCAGGCCAAG |
| | Nme2 base editor | AAGAAGAAGGGCGGCUCCGGCGGCGGCGAGGCCUCCCCCGCCUCC |
| | | GGCCCCCGGCACCUGAUGGACCCCCACAUCUUCACCUCCAACUUC |
| | | AACAACGGCAUCGGCCGGCACAAGACCUACCUGUGCUACGAGGUG |
| | | GAGCGGCUGGACAACGGCACCUCCGUGAAGAUGGACCAGCACCGG |
| | | GGCUUCCUGCACAACCAGGCCAAGAACCUGCUGUGCGGCUUCUAC |
| | | GGCCGGCACGCCGAGCUGCGGUUCCUGGACCUGGUGCCCUCCCUG |
| | | CAGCUGGACCCCGCCCAGAUCUACCGGGUGACCUGGUUCAUCUCC |
| | | UGGUCCCCCUGCUUCUCCUGGGGCUGCGCCGGCGAGGUGCGGGCC |
| | | UUCCUGCAGGAGAACACCCACGUGCGGCUGCGGAUCUUCGCCGCC |
| | | CGGAUCUACGACUACGACCCCCUGUACAAGGAGGCCCUGCAGAUG |
| | | CUGCGGGACGCCGGCGCCCAGGUGUCCAUCAUGACCUACGACGAG |
| | | UUCAAGCACUGCUGGGACACCUUCGUGGACCACCAGGGCUGCCCC |
| | | UUCCAGCCCUGGGACGGCCUGGACGAGCACUCCCAGGCCCUGUCC |
| | | GGCCGGCUGCGGGCCAUCCUGCAGAACCAGGGCAACggcaccaaggacuc |
| | | caccaaggacauccccgagacccccuccaaggacGCAGCGUUCAAACCAAAUcccauca |
| | | acuacauccugggccuggccaucggcaucgccuccgugggugggccaugguggagaucgacgagga |
| | | ggagaaccccauccggcugaucgaccugggggggguguucgagcgggccgaggugcccaagacc |
| | | ggcgacucccuggccauggcccggcggcuggcccgguccgugcggcggcugacccggcggcgggccc |
| | | accggcugcuggggcccggcggcugcugaaggggaggggugcugcaggccgccgacuucgacga |
| | | gaacggccugaucaagucccugcccaacacccccuggcagcugcgggccgccgcccuggaccggaagc |
| | | ugaccccccuggagugguccgccgugcugcugcaccugaucaagcaccggggcuaccugucccagcg |
| | | gaagaacgagggcgagaccgccgacaaggagcugggcgcccugcugaagggguggccaacaacgcc |
| | | cacgcccugcagaccggcgacuuccggacccccgccgagcuggcccugaacaaguucgagaaggaguc |
| | | cggccacauccggaaccagcggggcgacuacucccacaccuucucccggaaggaccugcaggccgagc |
| | | ugauccugcuguucgagaagcagaaggaguucggcaacccccacguguccggggccugaaggaggg |
| | | caucgagacccugcugaugacccagcggcccgcccuguccggcgacgccgugcagaagaugcugggc |
| | | cacugcaccuucgagcccgccgagcccaaggccgccaagaacaccuacaccgccgagcgguucaucug |
| | | gcugaccaagcugaacaaccugcggauccuggagcagggcuccgagcggccccugaccgacaccgagc |
| | | gggccacccugauggacgagcccuaccggaaguccaagcugaccuacgcccaggcccggaagcugcug |
| | | ggccuggaggacaccgccuucuucaagggccugcgguacggcaaggacaacgccgaggccuccaccc |
| | | ugauggagaugaaggccuaccacgccaucucccgggcccuggagaaggagggccugaaggacaagaa |
| | | guccccccugaaccuguccuccgagcugcaggacgagaucggcaccgccuucucccuguucaagaccg |
| | | acgaggacaucaccggccggcugaaggaccgggugcagcccgagauccuggaggcccugcugaagca |
| | | caucuccuucgacaaguucgugcagaucucccugaaggcccugggggaugugccccugauggag |
| | | cagggcaagcgguacgacgaggccugcgccgagaucuacggcgaccacuacggcaagaagaacaccga |
| | | ggagaagaucuaccugccccccauccccgccgacgagauccggaaccccguggugcugcgggcccug |
| | | ucccaggcccggaaggugaucaacggcguggugcgggguacggcucccccgcccggauccacaucg |
| | | agaccgcccgggaggugggcaaguccuucaaggaccggaaggagaucgagaagcggcaggaggagaa |
| | | ccggaaggaccgggagaaggccgccgccaaguuccgggaguacuuccccaacuucgugggcgagccc |
| | | aaguccaaggacauccugaagcugcggcuguacgagcagcagcacggcaagugccuguacuccggca |
| | | aggagaucaaccuggugcggcugaacgagaagggcuacguggagaucgaccacgcccugcccuucuc |
| | | ccggaccugggacgacuccuucaacaacaaggugcuggugcugggcuccgagaaccagaacaagggc |
| | | aaccagacccccuacgaguacuucaacggcaaggacaacucccgggaguggcaggaguucaaggcccg |
| | | gguggagaccucccgguucccccgguccaagaagcagcggauccugcugcagaaguucgacgaggac |
| | | ggcuucaaggagugcaaccugaacgacacccgguacgugaaccgcuuccugugccaguucguggccg |
| | | accacauccugcugaccggcaagggcaagcggcggguguucgccuccaacggccagaucaccaaccug |
| | | cugcggggcuucuggggccugcggaagguggggccgagaacgaccggcaccacgcccuggacgccg |
| | | uggugguggccugcuccaccguggccaugcagcagaagaucacccgguucguggguacaaggagau |
| | | gaacgccuucgacggcaagaccaucgacaaggagaccggcaaggugcugcaccagaagacccacuucc |
| | | cccagcccugggaguucuucgcccaggaggugaugauccggguguucggcaagcccgacggcaagcc |
| | | cgaguucgaggaggccgacacccccgagaagcugcggacccugcuggccgagaagcuguccucccgg |
| | | cccgaggccgugcacgaguacgugaccccccuguucgugucccgggcccccaaccggaagauguccg |
| | | gcgcccacaaggacacccugcgguccgccaagcgguucgugaagcacaacgagaagaucuccgugaag |
| | | cggguguggcugaccgagaucaagcuggccgaccuggagaacauggugaacuacaagaacggccggg |
| | | agaucgagcuguacgaggcccugaaggcccggcuggaggccuacggcggcaacgccaagcaggccuu |
| | | cgaccccaaggacaaccccuucuacaagaagggcggccagcuggugaaggccgugcggguggagaag |
| | | acccaggaguccggcgugcugcugaacaagaagaacgccuacaccaucgccgacaacggcgacauggu |
| | | gggguggacguguucugcaagguggacaagaagggcaagaaccaguacuucaucgugcccaucuac |
| | | gccuggcagguggccgagaacauccugcccgacaucgacugcaagggcuaccggaucgacgacuccu |
| | | acaccuucugcuucucccugcacaaguacgaccugaucgccuuccagaaggacgagaaguccaaggug |
| | | gaguucgccuacuacaucaacugcgacuccuccaacggccgguucuaccuggccuggcacgacaagg |
| | | gcuccaaggagcagcaguuccggaucuccacccagaaccuggugcugauccagaaguaccaggugaac |
| | | gagcugggcaaggagauccggcccugccggcugaagaagcggccccccgugcgguag |
| 311 | Amino acid | MDGSGGGSPKKKRKVEDKRPAATKKAGQAKKKKGGSGGGEASPASG |
| | sequence of | PRHLMDPHIFTSNFNNGIGRHKTYLCYEVERLDNGTSVKMDQHRGFLH |
| | Nme2 base editor | NQAKNLLCGFYGRHAELRFLDLVPSLQLDPAQIYRVTWFISWSPCFSW |
| | | GCAGEVRAFLQENTHVRLRIFAARIYDYDPLYKEALQMLRDAGAQVSI |
| | | MTYDEFKHCWDTFVDHQGCPFQPWDGLDEHSQALSGRLRAILQNQG |
| | | NGTKDSTKDIPETPSKDAAFKPNPINYILGLAIGIASVGWAMVEIDEEEN |
| | | PIRLIDLGVRVFERAEVPKTGDSLAMARRLARSVRRLTRRRAHRLLRA |
| | | RRLLKREGVLQAADFDENGLIKSLPNTPWQLRAAALDRKLTPLEWSA |
| | | VLLHLIKHRGYLSQRKNEGETADKELGALLKGVANNAHALQTGDFRT |
| | | PAELALNKFEKESGHIRNQRGDYSHTFSRKDLQAELILLFEKQKEFGNP |
| | | HVSGGLKEGIETLLMTQRPALSGDAVQKMLGHCTFEPAEPKAAKNTY |
| | | TAERFIWLTKLNNLRILEQGSERPLTDTERATLMDEPYRKSKLTYAQA |
| | | RKLLGLEDTAFFKGLRYGKDNAEASTLMEMKAYHAISRALEKEGLKD |
| | | KKSPLNLSSELQDEIGTAFSLFKTDEDITGRLKDRVQPEILEALLKHISFD |
| | | KFVQISLKALRRIVPLMEQGKRYDEACAEIYGDHYGKKNTEEKIYLPPI |
| | | PADEIRNPVVLRALSQARKVINGVVRRYGSPARIHIETAREVGKSFKDR |
| | | KEIEKRQEENRKDREKAAAKFREYFPNFVGEPKSKDILKLRLYEQQHG |
| | | KCLYSGKEINLVRLNEKGYVEIDHALPFSRTWDDSFNNKVLVLGSENQ |
| | | NKGNQTPYEYFNGKDNSREWQEFKARVETSRFPRSKKQRILLQKFDED |
| | | GFKECNLNDTRYVNRFLCQFVADHILLTGKGKRRVFASNGQITNLLRG |
| | | FWGLRKVRAENDRHHALDAVVVACSTVAMQQKITRFVRYKEMNAFD |
| | | GKTIDKETGKVLHQKTHFPQPWEFFAQEVMIRVFGKPDGKPEFEEADT |
| | | PEKLRTLLAEKLSSRPEAVHEYVTPLFVSRAPNRKMSGAHKDTLRSAK |
| | | RFVKHNEKISVKRVWLTEIKLADLENMVNYKNGREIELYEALKARLEA |
| | | YGGNAKQAFDPKDNPFYKKGGQLVKAVRVEKTQESGVLLNKKNAYT |
| | | IADNGDMVRVDVFCKVDKKGKNQYFIVPIYAWQVAENILPDIDCKGY |
| | | RIDDSYTFCFSLHKYDLIAFQKDEKSKVEFAYYINCDSSNGRFYLAWH |
| | | DKGSKEQQFRISTQNLVLIQKYQVNELGKEIRPCRLKKRPPVR* |
| 312 | Exemplary | mN*mN*mN*mNmNNNmNmNNmNNmNNNNNmNNNNmNNNmGUUG |
| | modified Nme | mUmAmGmCUCCCmUmGmAmAmAmCmCGUUmGmCUAmCAAUAAG |
| | guide sgRNA | mGmCCmGmUmCmGmAmAmAmGmAmUGUGCmCGmCAAmCGCUCU |
| | | mGmCCmUmUmCmUGGCAUCG*mU*mU |
| 313 | Exemplary | mN*mN*mN*mNmNNNmNmNNmNNmNNNNNmNNNNmNNNmGUUG |
| | modified Nme | mUmAmGmCUCCCmUmGmAmAmAmCmCGUUmGmCUAmCAAU*AA |
| | guide sgRNA | GmGmCCmGmUmCmGmAmAmAmGmAmUGUGCmCGmCAAmCGmCm |
| | | UmCmUmGmCCmUmUmCmUGGCAUCG*mU*mU |
| 314 | Exemplary | mN*mN*mN*mNmNNNmNmNNmNNmNNNNNmNNNNmNNNmGUUG |
| | modified Nme | mUmAmGmCUCCCmUmGmAmAmAmCmCGUUmGmCUAmCAAUAAG |
| | guide sgRNA | mGmCCmGmUmCmGmAmAmAmGmAmUGUGCmCGmCAAmCGmCmU |
| | | mCmUmGmCCmUmUmCmUGGCAUCG*mU*mU |

TABLE 19
Exemplary SpyCas9 sgRNA conserved portion (SEQ ID NO: 226)

| 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 | 26 | 27 | 28 | 29 | 30 |
|---|---|---|---|---|---|---|---|---|---|---|---|---|---|---|---|---|---|---|---|---|---|---|---|---|---|---|---|---|---|
| G | U | U | U | U | A | G | A | G | C | U | A | G | A | A | A | U | A | G | C | A | A | G | U | U | A | A | A | A | U |
| LS1-LS6 | | | | | | B1-B2 | | US1-US12 | | | | | | | | | | | | B2-B6 | | | | LS7-LS12 | | | | | |
| 31 | 32 | 33 | 34 | 35 | 36 | 37 | 38 | 39 | 40 | 41 | 42 | 43 | 44 | 45 | 46 | 47 | 48 | 49 | 50 | 51 | 52 | 53 | 54 | 55 | 56 | 57 | 58 | 59 | 60 |
| A | A | G | G | C | U | A | G | U | C | C | G | U | U | A | U | C | A | A | C | U | U | G | A | A | A | A | A | G | U |

| Nexus | | | | | | | | | H1-1 through H1-12 | | | | | | |
|---|---|---|---|---|---|---|---|---|---|---|---|---|---|---|---|
| 61 | 62 | 63 | 64 | 65 | 66 | 67 | 68 | 69 | 70 | 71 | 72 | 73 | 74 | 75 | 76 |
| G | G | C | A | C | C | G | A | G | U | C | G | G | U | G | C |

| | |
|---|---|
| N | H2-1 through H2-15 |

TABLE 20
Exemplary NmeCas9 sgRNA conserved portion (SEQ ID NO: 400 (“Exemplary NmeCas9 sgRNA-1”)

| 1-24 | 25 | 26 | 27 | 28 | 29 | 30 | 31 | 32 | 33 |
|---|---|---|---|---|---|---|---|---|---|
| NNNNNNNNNNNNNNNNNNNNNNNN | G | U | U | G | U | A | G | C | U |

| | |
|---|---|
| | Lower stem |
| Guide region | Repeat/Anti-Repeat region |

| 34 | 35 | 36 | 37 | 38 | 39 | 40 | 41 | 42 | 43 | 44 | 45 | 46 | 47 | 48 |
|---|---|---|---|---|---|---|---|---|---|---|---|---|---|---|
| C | C | C | U | U | U | C | U | C | A | U | U | U | C | G |

| | |
|---|---|
| Lower stem | Upper stem |

Repeat/Anti-Repeat region

| 49 | 50 | 51 | 52 | 53 | 54 | 55 | 56 | 57 | 58 | 59 | 60 | 61 | 62 | 63 | 64 | 65 | 66 | 67 | 68 | 69 | 70 | 71 | 72 | 73 | 74 | 75 | 76 | 77 |
|---|---|---|---|---|---|---|---|---|---|---|---|---|---|---|---|---|---|---|---|---|---|---|---|---|---|---|---|---|
| G | A | A | A | C | G | A | A | A | U | G | A | G | A | A | C | C | G | U | U | G | C | U | A | C | A | A | U | A |

| | | |
|---|---|---|
| Loop | Upper stem | Lower stem |

Repeat/Anti-Repeat region

| 78 | 79 | 80 | 81 | 82 | 83 | 84 | 85 | 86 | 87 | 88 | 89 | 90 | 91 | 92 | 93 | 94 |
|---|---|---|---|---|---|---|---|---|---|---|---|---|---|---|---|---|
| A | G | G | C | C | G | U | C | U | G | A | A | A | A | G | A | U |

| | | |
|---|---|---|
| Stem | Loop | Stem (96: unpaired) |

Hairpin 1

| 95 | 96 | 97 | 98 | 99 | 100 | 101 | 102 | 103 | 104 | 105 | 106 | 107 | 108 |
|---|---|---|---|---|---|---|---|---|---|---|---|---|---|
| G | U | G | C | C | G | C | A | A | C | G | C | U | C |

| | | |
|---|---|---|
| Stem (96: unpaired) | Lower stem | Bulge |

| | |
|---|---|
| Hairpin 1 | Hairpin 2 |

| 109 | 110 | 111 | 112 | 113 | 114 | 115 | 116 | 117 | 118 | 119 | 120 | 121 | 122 |
|---|---|---|---|---|---|---|---|---|---|---|---|---|---|
| U | G | C | C | C | C | U | U | A | A | A | G | C | U |

| | |
|---|---|
| Upper Stem | Loop |

Hairpin 2

| 123 | 124 | 125 | 126 | 127 | 128 | 129 | 130 | 131 | 132 | 133 | 134 |
|---|---|---|---|---|---|---|---|---|---|---|---|
| U | C | U | G | C | U | U | U | A | A | G | G |

| | |
|---|---|
| Loop | Upper Stem |

Hairpin 2

| 135 | 136 | 137 | 138 | 139 | 140 | 141 | 142 | 143 | 144 | 145 |
|---|---|---|---|---|---|---|---|---|---|---|
| G | G | C | A | U | C | G | U | U | U | A |

| | | |
|---|---|---|
| Upper Stem | Bulge | Lower Stem |

| | |
|---|---|
| Hairpin 2 | Tail |

TABLE 21
Additional Exemplary Nme Guide RNAs

| | | | | | Genomic |
|---|---|---|---|---|---|
| | | Guide | Exemplary Guide RNA Full | | Coordinates |
| Guide ID | Target | Sequence | Sequence | Exemplary Guide RNA Modified Sequence | (hg38) |
| G013006 | TRAC | CUCUCAGCUG | CUCUCAGCUGGUACACGGCAG | mC*mU*mC*UCAGCUGGUACACGGCAGUUU | chr14: 22547524- |
| | | GUACACGGC | UUUUAGAGCUAGAAAUAGCAA | UAGAmGmCmUmAmGmAmAmAmUmAmGmC | 22547544 |
| | | A (SEQ ID NO: | GUUAAAAUAAGGCUAGUCCGU | AAGUUAAAAUAAGGCUAGUCCGUUAUCAm | |
| | | 315) | UAUCAACUUGAAAAAGUGGCA | AmCmUmUmGmAmAmAmAmAmGmUmGmGm | |
| | | | CCGAGUCGGUGCUUUU (SEQ ID | CmAmCmCmGmAmGmUmCmGmGmUmGmCm | |
| | | | NO: 316) | U*mU*mU*mU (SEQ ID NO: 317) | |
| G013675 | CIITA | CCCCCGGACG | CCCCCGGACGGUUCAAGCAAG | mC*mC*mC*CCGGACGGUUCAAGCAAGUUU | chr16: 10906853- |
| | | GUUCAAGCA | UUUUAGAGCUAGAAAUAGCAA | UAGAmGmCmUmAmGmAmAmAmUmAmGmC | 10906873 |
| | | A (SEQ ID NO: | GUUAAAAUAAGGCUAGUCCGU | AAGUUAAAAUAAGGCUAGUCCGUUAUCAm | |
| | | 318) | UAUCAACUUGAAAAAGUGGCA | AmCmUmUmGmAmAmAmAmAmGmUmGmGm | |
| | | | CCGAGUCGGUGCUUUU (SEQ ID | CmAmCmCmGmAmGmUmCmGmGmUmGmCm | |
| | | | NO: 319) | U*mU*mU*mU (SEQ ID NO: 320) | |
| G014832 | TRBC1 | GGCUCUCGG | GGCUCUCGGAGAAUGACGAGG | mG*mG*mC*UCUCGGAGAAUGACGAGGUUU | chr7: 142791996- |
| | | AGAAUGACG | UUUUAGAGCUAGAAAUAGCAA | UAGAmGmCmUmAmGmAmAmAmUmAmGmC | 142792016 |
| | | AG (SEQ ID | GUUAAAAUAAGGCUAGUCCGU | AAGUUAAAAUAAGGCUAGUCCGUUAUCAm | |
| | | NO: 321) | UAUCAACUUGAAAAAGUGGCA | AmCmUmUmGmAmAmAmAmAmGmUmGmGm | |
| | | | CCGAGUCGGUGCUUUU (SEQ ID | CmAmCmCmGmAmGmUmCmGmGmUmGmCm | |
| | | | NO: 322) | U*mU*mU*mU (SEQ ID NO: 323) | |
| G016239 | TRBC1 | GGCCUCGGCG | GGCCUCGGCGCUGACGAUCUG | mG*mG*mC*CUCGGCGCUGACGAUCUGUUU | chr7: 142792047- |
| | | CUGACGAUC | UUUUAGAGCUAGAAAUAGCAA | UAGAmGmCmUmAmGmAmAmAmUmAmGmC | 142792067 |
| | | U (SEQ ID NO: | GUUAAAAUAAGGCUAGUCCGU | AAGUUAAAAUAAGGCUAGUCCGUUAUCAm | |
| | | 324) | UAUCAACUUGAAAAAGUGGCA | AmCmUmUmGmAmAmAmAmAmGmUmGmGm | |
| | | | CCGAGUCGGUGCUUUU (SEQ ID | CmAmCmCmGmAmGmUmCmGmGmUmGmCm | |
| | | | NO: 325) | U*mU*mU*mU (SEQ ID NO: 326) | |
| G018995 | HLA-A | ACAGCGACGC | ACAGCGACGCCGCGAGCCAGG | mA*mC*mA*GCGACGCCGCGAGCCAGGUUU | chr6: 29942864- |
| | | CGCGAGCCAG | UUUUAGAGCUAGAAAUAGCAA | UAGAmGmCmUmAmGmAmAmAmUmAmGmC | 29942884 |
| | | (SEQ ID NO: | GUUAAAAUAAGGCUAGUCCGU | AAGUUAAAAUAAGGCUAGUCCGUUAUCAm | |
| | | 327) | UAUCAACUUGAAAAAGUGGCA | AmCmUmUmGmAmAmAmAmAmGmUmGmGm | |
| | | | CCGAGUCGGUGCUUUU (SEQ ID | CmAmCmCmGmAmGmUmCmGmGmUmGmCm | |
| | | | NO: 328) | U*mU*mU*mU (SEQ ID NO: 329) | |
| G021469 | TRAC | AUAUCCAGA | AUAUCCAGAACCCUGACCCUG | mA*mU*mA*mUmCCAmGmAAmCCmCUGACm | chr14: 22547505- |
| | | ACCCUGACCC | CCGGUUGUAGCUCCCUGAAAC | CCUGmCCGmGUUGmUmAmGmCUCCCmUmG | 22547529 |
| | | UGCCG (SEQ | CGUUGCUACAAUAAGGCCGUC | mAmAmAmCmCGUUmGmCUAmCAAU*AAGm | |
| | | ID NO: 330) | GAAAGAUGUGCCGCAACGCUC | GmCCmGmUmCmGmAmAmAmGmAmUGUGCm | |
| | | | UGCCUUCUGGCAUCGUU (SEQ | CGmCAAmCGCUCUmGmCCmUmUmCmUGGC | |
| | | | ID NO: 331) | AUCG*mU*mU (SEQ ID NO: 332) | |
| G023520 | TRAC | UUCAAAACC | UUCAAAACCUGUCAGUGAUUG | mU*mU*mC*AAAACCUGUCAGUGAUUGUUU | chr14: 22550571- |
| | | UGUCAGUGA | UUUUAGAGCUAGAAAUAGCAA | UAGAmGmCmUmAmGmAmAmAmUmAmGmC | 22550591 |
| | | UU (SEQ ID | GUUAAAAUAAGGCUAGUCCGU | AAGUUAAAAUAAGGCUAGUCCGUUAUCACG | |
| | | NO: 333) | UAUCACGAAAGGGCACCGAGU | AAAGGGCACCGAGUCGGmUmGmC*mU (SEQ | |
| | | | CGGUGCU (SEQ ID NO: 334) | ID NO: 335) | |
| G023521 | CIITA | CGCCCAGGUC | CGCCCAGGUCCUCACGUCUGG | mC*mG*mC*CCAGGUCCUCACGUCUGGUUUU | chr16: 10907539- |
| | | CUCACGUCUG | UUUUAGAGCUAGAAAUAGCAA | AGAmGmCmUmAmGmAmAmAmUmAmGmCA | 10907559 |
| | | (SEQ ID NO: | GUUAAAAUAAGGCUAGUCCGU | AGUUAAAAUAAGGCUAGUCCGUUAUCACGA | |
| | | 336) | UAUCACGAAAGGGCACCGAGU | AAGGGCACCGAGUCGGmUmGmC*mU (SEQ | |
| | | | CGGUGCU (SEQ ID NO: 337) | ID NO: 338) | |
| G023523 | HLA-A | GCUGCAGCGC | GCUGCAGCGCACGGGUACCAG | mG*mC*mU*GCAGCGCACGGGUACCAGUUU | chr6: 29943529- |
| | | ACGGGUACC | UUUUAGAGCUAGAAAUAGCAA | UAGAmGmCmUmAmGmAmAmAmUmAmGmC | 29943549 |
| | | A (SEQ ID NO: | GUUAAAAUAAGGCUAGUCCGU | AAGUUAAAAUAAGGCUAGUCCGUUAUCACG | |
| | | 339) | UAUCACGAAAGGGCACCGAGU | AAAGGGCACCGAGUCGGmUmGmC*mU (SEQ | |
| | | | CGGUGCU (SEQ ID NO: 340) | ID NO: 341) | |
| G023524 | TRBC2 | CCACACCCAA | CCACACCCAAAAGGCCACACG | mC*mC*mA*CACCCAAAAGGCCACACGUUUU | chr7: 142801104- |
| | | AAGGCCACAC | UUUUAGAGCUAGAAAUAGCAA | AGAmGmCmUmAmGmAmAmAmUmAmGmCA | 142801124 |
| | | (SEQ ID NO: | GUUAAAAUAAGGCUAGUCCGU | AGUUAAAAUAAGGCUAGUCCGUUAUCACGA | |
| | | 342) | UAUCACGAAAGGGCACCGAGU | AAGGGCACCGAGUCGGmUmGmC*mU (SEQ | |
| | | | CGGUGCU (SEQ ID NO: 343) | ID NO: 344) | |
| G026584 | CIITA | UCAAAGUAC | UCAAAGUACCCUACAGGAGGA | mU*mC*mA*mAmAGUmAmCCmCUmACAGGm | chr16: 10907504- |
| | | CCUACAGGA | CCAGUUGUAGCUCCCUGAAAC | AGGAmCCAmGUUGmUmAmGmCUCCCmUmG | 10907528 |
| | | GGACCA (SEQ | CGUUGCUACAAUAAGGCCGUC | mAmAmAmCmCGUUmGmCUAmCAAU*AAGm | |
| | | ID NO: 345) | GAAAGAUGUGCCGCAACGCUC | GmCCmGmUmCmGmAmAmAmGmAmUGUGCm | |
| | | | UGCCUUCUGGCAUCGUU (SEQ | CGmCAAmCGCUCUmGmCCmUmUmCmUGGC | |
| | | | ID NO: 346) | AUCG*mU*mU (SEQ ID NO: 347) | |
| G027891 | TRAC | UUCAAAACC | UUCAAAACCUGUCAGUGAUUG | mU*mU*mC*AAAACCUGUCAGUGAUUGUUU | chr14: 22550571- |
| | | UGUCAGUGA | UUUUAGAGCUAGAAAUAGCAA | UAGAmGmCmUmAmGmAmAmAmUmAmGmC | 22550591 |
| | | UU (SEQ ID | GUUAAAAUAAGGCUAGUCCGU | AAGUUAAAAUAAGGCUAGUCCGUUAUCACG | |
| | | NO: 348) | UAUCACGAAAGGGCACCGAGU | AAAGGGCACCGAGUCGGmU*mG*mC*mU | |
| | | | CGGUGCU (SEQ ID NO: 349) | (SEQ ID NO: 350) | |
| G027904 | TRBC2 | CCACACCCAA | CCACACCCAAAAGGCCACACG | mC*mC*mA*CACCCAAAAGGCCACACGUUUU | chr7: 142801104- |
| | | AAGGCCACAC | UUUUAGAGCUAGAAAUAGCAA | AGAmGmCmUmAmGmAmAmAmUmAmGmCA | 142801124 |
| | | | GUUAAAAUAAGGCUAGUCCGU | AGUUAAAAUAAGGCUAGUCCGUUAUCACGA | |
| | | (SEQ ID NO: | UAUCACGAAAGGGCACCGAGU | AAGGGCACCGAGUCGGmU*mG*mC*mU (SEQ | |
| | | 351) | CGGUGCU (SEQ ID NO: 352) | ID NO: 353) | |
| G028535 | CIITA | CGCCCAGGUC | CGCCCAGGUCCUCACGUCUGG | mC*mG*mC*CCAGGUCCUCACGUCUGGUUUU | chr16: 10907539- |
| | | CUCACGUCUG | UUUUAGAGCUAGAAAUAGCAA | AGAmGmCmUmAmGmAmAmAmUmAmGmCA | 10907559 |
| | | (SEQ ID NO: | GUUAAAAUAAGGCUAGUCCGU | AGUUAAAAUAAGGCUAGUCCGUUAUCACGA | |
| | | 354) | UAUCACGAAAGGGCACCGAGU | AAGGGCACCGAGUCGGmU*mG*mC*mU (SEQ | |
| | | | CGGUGCU (SEQ ID NO: 355) | ID NO: 356) | |
| G028536 | HLA-A | GCUGCAGCGC | GCUGCAGCGCACGGGUACCAG | mG*mC*mU*GCAGCGCACGGGUACCAGUUU | chr6: 29943529- |
| | | ACGGGUACC | UUUUAGAGCUAGAAAUAGCAA | UAGAmGmCmUmAmGmAmAmAmUmAmGmC | 29943549 |
| | | A (SEQ ID NO: | GUUAAAAUAAGGCUAGUCCGU | AAGUUAAAAUAAGGCUAGUCCGUUAUCACG | |
| | | 357) | UAUCACGAAAGGGCACCGAGU | AAAGGGCACCGAGUCGGmU*mG*mC*mU | |
| | | | CGGUGCU (SEQ ID NO: 358) | (SEQ ID NO: 359) | |
| G028907 | HLA-A | UCCUGCUCUA | UCCUGCUCUAUCCACGGCGCC | mU*mC*mC*mUmGCUmCmUAmUCmCACGGm | chr6: 29942895- |
| | | UCCACGGCGC | CGCGUUGUAGCUCCCUGAAAC | CGCCmCGCmGUUGmUmAmGmCUCCCmUmG | 29942919 |
| | | CCGC (SEQ ID | CGUUGCUACAAUAAGGCCGUC | mAmAmAmCmCGUUmGmCUAmCAAU*AAGm | |
| | | NO: 360) | GAAAGAUGUGCCGCAACGCUC | GmCCmGmUmCmGmAmAmAmGmAmUGUGCm | |
| | | | UGCCUUCUGGCAUCGUU (SEQ | CGmCAAmCGCUCUmGmCCmUmUmCmUGGC | |
| | | | ID NO: 361) | AUCG*mU*mU (SEQ ID NO: 362) | |
| G028986 | TRBC1 | GUGUCCUACC | GUGUCCUACCAGCAAGGGGUC | mG*mU*mG*mUmCCUmAmCCmAGmCAAGGm | chr7: 142792690- |
| | | AGCAAGGGG | CUGGUUGUAGCUCCCUGAAAC | GGUCmCUGmGUUGmUmAmGmCUCCCmUmG | 142792714 |
| | | UCCUG (SEQ | CGUUGCUACAAUAAGGCCGUC | mAmAmAmCmCGUUmGmCUAmCAAU*AAGm | |
| | | ID NO: 363) | GAAAGAUGUGCCGCAACGCUC | GmCCmGmUmCmGmAmAmAmGmAmUGUGCm | |
| | | | UGCCUUCUGGCAUCGUU (SEQ | CGmCAAmCGCUCUmGmCCmUmUmCmUGGC | |
| | | | ID NO: 364) | AUCG*mU*mU (SEQ ID NO: 365) | |
| G028918 | HLA-A | GCUCUAUCCA | GCUCUAUCCACGGCGCCCGCG | mG*mC*mU*mCmUAUmCmCAmCGmGCGCCm | chr6: 29942891- |
| | | CGGCGCCCGC | GCUGUUGUAGCUCCCUGAAAC | CGCGmGCUmGUUGmUmAmGmCUCCCmUmG | 29942915 |
| | | GGCU (SEQ ID | CGUUGCUACAAUAAGGCCGUC | mAmAmAmCmCGUUmGmCUAmCAAU*AAGm | |
| | | NO: 366) | GAAAGAUGUGCCGCAACGCUC | GmCCmGmUmCmGmAmAmAmGmAmUGUGCm | |
| | | | UGCCUUCUGGCAUCGUU (SEQ | CGmCAAmCGCUCUmGmCCmUmUmCmUGGC | |
| | | | ID NO: 367) | AUCG*mU*mU (SEQ ID NO: 368) | |
| G034202 | HLA-A | GCUCUAUCCA | GCUCUAUCCACGGCGCCCGCG | mG*mC*mU*mCmUAUmCmCAmCGmGCGCCm | chr6: 29942891- |
| | | CGGCGCCCGC | GCUGUUGUAGCUCCCUGAAAC | CGCGmGCUmGUUGmUmAmGmCUCCCmUmG | 29942915 |
| | | GGCU (SEQ ID | CGUUGCUACAAUAAGGCCGUC | mAmAmAmCmCGUUmGmCUAmCAAUAAGmG | |
| | | NO: 366) | GAAAGAUGUGCCGCAACGCUC | mCCmGmUmCmGmAmAmAmGmAmUGUGCmC | |
| | | | UGCCUUCUGGCAUCGUU (SEQ | GmCAAmCGCUCUmGmCCmUmUmCmUGGCA | |
| | | | ID NO: 367) | UCG*mU*mU (SEQ ID NO: 369) | |
| G028913 | HLA-A | CACUCACCCG | CACUCACCCGCCCAGGUCUGG | mC*mA*mC*mUmCACmCmCGmCCmCAGGUm | chr6: 29942609- |
| | | CCCAGGUCUG | GUCGUUGUAGCUCCCUGAAAC | CUGGmGUCmGUUGmUmAmGmCUCCCmUmG | 29942633 |
| | | GGUC (SEQ ID | CGUUGCUACAAUAAGGCCGUC | mAmAmAmCmCGUUmGmCUAmCAAU*AAGm | |
| | | NO: 370) | GAAAGAUGUGCCGCAACGCUC | GmCCmGmUmCmGmAmAmAmGmAmUGUGCm | |
| | | | UGCCUUCUGGCAUCGUU (SEQ | CGmCAAmCGCUCUmGmCCmUmUmCmUGGC | |
| | | | ID NO: 371) | AUCG *mU*mU (SEQ ID NO: 372) | |
| G034617 | HLA-A | CACUCACCCG | CACUCACCCGCCCAGGUCUGG | mC*mA*mC*mUmCACmCmCGmCCmCAGGUm | chr6: 29942609- |
| | | CCCAGGUCUG | GUCGUUGUAGCUCCCUGAAAC | CUGGmGUCmGUUGmUmAmGmCUCCCmUmG | 29942633 |
| | | GGUC (SEQ ID | CGUUGCUACAAUAAGGCCGUC | mAmAmAmCmCGUUmGmCUAmCAAUAAGmG | |
| | | NO: 370) | GAAAGAUGUGCCGCAACGCUC | mCCmGmUmCmGmAmAmAmGmAmUGUGCmC | |
| | | | UGCCUUCUGGCAUCGUU (SEQ | GmCAAmCGCUCUmGmCCmUmUmCmUGGCA | |
| | | | ID NO: 371) | UCG*mU*mU (SEQ ID NO: 373) | |
| G028943 | TRAC | AAAACCUGU | AAAACCUGUCAGUGAUUGGGU | mA*mA*mA*mAmCCUmGmUCmAGmUGAUUm | chr14: 22550574- |
| | | CAGUGAUUG | UCCGUUGUAGCUCCCUGAAAC | GGGUmUCCmGUUGmUmAmGmCUCCCmUmG | 22550598 |
| | | GGUUCC (SEQ | CGUUGCUACAAUAAGGCCGUC | mAmAmAmCmCGUUmGmCUAmCAAU*AAGm | |
| | | ID NO: 374) | GAAAGAUGUGCCGCAACGCUC | GmCCmGmUmCmGmAmAmAmGmAmUGUGCm | |
| | | | UGCCUUCUGGCAUCGUU (SEQ | CGmCAAmCGCUCUmGmCCmUmUmCmUGGC | |
| | | | ID NO: 375) | AUCG*mU*mU (SEQ ID NO: 376) | |
| G034982 | TRAC | AAAACCUGU | AAAACCUGUCAGUGAUUGGGU | mA*mA*mA*mAmCCUmGmUCmAGmUGAUUm | chr14: 22550574- |
| | | CAGUGAUUG | UCCGUUGUAGCUCCCUGAAAC | GGGUmUCCmGUUGmUmAmGmCUCCCmUmG | 22550598 |
| | | GGUUCC (SEQ | CGUUGCUACAAUAAGGCCGUC | mAmAmAmCmCGUUmGmCUAmCAAUAAGmG | |
| | | ID NO: 374) | GAAAGAUGUGCCGCAACGCUC | mCCmGmUmCmGmAmAmAmGmAmUGUGCmC | |
| | | | UGCCUUCUGGCAUCGUU (SEQ | GmCAAmCGCUCUmGmCCmUmUmCmUGGCA | |
| | | | ID NO: 375) | UCG*mU*mU (SEQ ID NO: 377) | |
| G028939 | TRAC | UUAGGUUCG | UUAGGUUCGUAUCUGUAAAAC | mU*mU*mA*mGmGUUmCmGUmAUmCUGUA | chr14: 22550544- |
| | | UAUCUGUAA | CAAGUUGUAGCUCCCUGAAAC | mAAACmCAAmGUUGmUmAmGmCUCCCmUm | 22550568 |
| | | AACCAA (SEQ | CGUUGCUACAAUAAGGCCGUC | GmAmAmAmCmCGUUmGmCUAmCAAU*AAG | |
| | | ID NO: 378) | GAAAGAUGUGCCGCAACGCUC | mGmCCmGmUmCmGmAmAmAmGmAmUGUGC | |
| | | | UGCCUUCUGGCAUCGUU (SEQ | mCGmCAAmCGCUCUmGmCCmUmUmCmUGG | |
| | | | ID NO: 379) | CAUCG*mU*mU (SEQ ID NO: 380) | |
| G034981 | TRAC | UUAGGUUCG | UUAGGUUCGUAUCUGUAAAAC | mU*mU*mA*mGmGUUmCmGUmAUmCUGUA | chr14: 22550544- |
| | | UAUCUGUAA | CAAGUUGUAGCUCCCUGAAAC | mAAACmCAAmGUUGmUmAmGmCUCCCmUm | 22550568 |
| | | AACCAA (SEQ | CGUUGCUACAAUAAGGCCGUC | GmAmAmAmCmCGUUmGmCUAmCAAUAAGm | |
| | | ID NO: 378) | GAAAGAUGUGCCGCAACGCUC | GmCCmGmUmCmGmAmAmAmGmAmUGUGCm | |
| | | | UGCCUUCUGGCAUCGUU (SEQ | CGmCAAmCGCUCUmGmCCmUmUmCmUGGC | |
| | | | ID NO: 379) | AUCG*mU*mU (SEQ ID NO: 381) | |
| G028986 | TRBC1 | GUGUCCUACC | GUGUCCUACCAGCAAGGGGUC | mG*mU*mG*mUmCCUmAmCCmAGmCAAGGm | chr7: 142792690- |
| | | AGCAAGGGG | CUGGUUGUAGCUCCCUGAAAC | GGUCmCUGmGUUGmUmAmGmCUCCCmUmG | 142792714 |
| | | UCCUG (SEQ | CGUUGCUACAAUAAGGCCGUC | mAmAmAmCmCGUUmGmCUAmCAAU*AAGm | |
| | | ID NO: 363) | GAAAGAUGUGCCGCAACGCUC | GmCCmGmUmCmGmAmAmAmGmAmUGUGCm | |
| | | | UGCCUUCUGGCAUCGUU (SEQ | CGmCAAmCGCUCUmGmCCmUmUmCmUGGC | |
| | | | ID NO: 364) | AUCG*mU*mU (SEQ ID NO: 365) | |
| G034618 | TRBC1 | GUGUCCUACC | GUGUCCUACCAGCAAGGGGUC | mG*mU*mG*mUmCCUmAmCCmAGmCAAGGm | chr7: 142792690- |
| | | AGCAAGGGG | CUGGUUGUAGCUCCCUGAAAC | GGUCmCUGmGUUGmUmAmGmCUCCCmUmG | 142792714 |
| | | UCCUG (SEQ | CGUUGCUACAAUAAGGCCGUC | mAmAmAmCmCGUUmGmCUAmCAAUAAGmG | |
| | | ID NO: 363) | GAAAGAUGUGCCGCAACGCUC | mCCmGmUmCmGmAmAmAmGmAmUGUGCmC | |
| | | | UGCCUUCUGGCAUCGUU (SEQ | GmCAAmCGCUCUmGmCCmUmUmCmUGGCA | |
| | | | ID NO: 364) | UCG*mU*mU (SEQ ID NO: 382) | |
| G026584 | CIITA | UCAAAGUAC | UCAAAGUACCCUACAGGAGGA | mUmC*mA*mAmAGUmAmCCmCUmACAGGm | chr16: 10907504- |
| | | CCUACAGGA | CCAGUUGUAGCUCCCUGAAAC | AGGAmCCAmGUUGmUmAmGmCUCCCmUmG | 10907528 |
| | | GGACCA (SEQ | CGUUGCUACAAUAAGGCCGUC | mAmAmAmCmCGUUmGmCUAmCAAU*AAGm | |
| | | ID NO: 345) | GAAAGAUGUGCCGCAACGCUC | GmCCmGmUmCmGmAmAmAmGmAmUGUGCm | |
| | | | UGCCUUCUGGCAUCGUU (SEQ | CGmCAAmCGCUCUmGmCCmUmUmCmUGGC | |
| | | | ID NO: 346) | AUCG*mU*mU (SEQ ID NO: 347) | |
| G034201 | CIITA | UCAAAGUAC | UCAAAGUACCCUACAGGAGGA | mU*mC*mA*mAmAGUmAmCCmCUmACAGGm | chr16: 10907504- |
| | | CCUACAGGA | CCAGUUGUAGCUCCCUGAAAC | AGGAmCCAmGUUGmUmAmGmCUCCCmUmG | 10907528 |
| | | GGACCA (SEQ | CGUUGCUACAAUAAGGCCGUC | mAmAmAmCmCGUUmGmCUAmCAAUAAGmG | |
| | | ID NO: 345) | GAAAGAUGUGCCGCAACGCUC | mCCmGmUmCmGmAmAmAmGmAmUGUGCmC | |
| | | | UGCCUUCUGGCAUCGUU (SEQ | GmCAAmCGCUCUmGmCCmUmUmCmUGGCA | |
| | | | ID NO: 346) | UCG*mU*mU (SEQ ID NO: 383) | |
| G029131 | CIITA | AGCUGCCGU | AGCUGCCGUUCUGCCCAGUCC | mA*mG*mC*mUmGCCmGmUUmCUmGCCCAm | chr16: 10906643- |
| | | UCUGCCCAGU | GGGGUUGUAGCUCCCUGAAAC | GUCCmGGGmGUUGmUmAmGmCUCCCmUmG | 10906667 |
| | | CCGGG (SEQ | CGUUGCUACAAUAAGGCCGUC | mAmAmAmCmCGUUmGmCUAmCAAU*AAGm | |
| | | ID NO: 384) | GAAAGAUGUGCCGCAACGCUC | GmCCmGmUmCmGmAmAmAmGmAmUGUGCm | |
| | | | UGCCUUCUGGCAUCGUU (SEQ | CGmCAAmCGCUCUmGmCCmUmUmCmUGGC | |
| | | | ID NO: 385) | AUCG*mU*mU (SEQ ID NO: 386) | |
| G034619 | CIITA | AGCUGCCGU | AGCUGCCGUUCUGCCCAGUCC | mA*mG*mC*mUmGCCmGmUUmCUmGCCCAm | chr16: 10906643- |
| | | UCUGCCCAGU | GGGGUUGUAGCUCCCUGAAAC | GUCCmGGGmGUUGmUmAmGmCUCCCmUmG | 10906667 |
| | | CCGGG (SEQ | CGUUGCUACAAUAAGGCCGUC | mAmAmAmCmCGUUmGmCUAmCAAUAAGmG | |
| | | ID NO: 384) | GAAAGAUGUGCCGCAACGCUC | mCCmGmUmCmGmAmAmAmGmAmUGUGCmC | |
| | | | UGCCUUCUGGCAUCGUU (SEQ | GmCAAmCGCUCUmGmCCmUmUmCmUGGCA | |
| | | | ID NO: 385) | UCG*mU*mU (SEQ ID NO: 387) | |
| G032794 | HLA-B | UCUGGGAAA | UCUGGGAAAGGAGGGGAAGAU | mU*mC*mU*mGmGGAmAmAGmGAmGGGGA | chr6: 31355222- |
| | | GGAGGGGAA | GAGGUUGUAGCUCCCUGAAAC | mAGAUmGAGmGUUGmUmAmGmCUCCCmUm | 31355246 |
| | | GAUGAG (SEQ | CGUUGCUACAAUAAGGCCGUC | GmAmAmAmCmCGUUmGmCUAmCAAU*AAG | |
| | | ID NO: 388) | GAAAGAUGUGCCGCAACGCUC | mGmCCmGmUmCmGmAmAmAmGmAmUGUGC | |
| | | | UGCCUUCUGGCAUCGUU (SEQ | mCGmCAAmCGCUCUmGmCCmUmUmCmUGG | |
| | | | ID NO: 389) | CAUCG*mU*mU (SEQ ID NO: 390) | |
| G034208 | HLA-B | UCUGGGAAA | UCUGGGAAAGGAGGGGAAGAU | mU*mC*mU*mGmGGAmAmAGmGAmGGGGA | chr6: 31355222- |
| | | GGAGGGGAA | GAGGUUGUAGCUCCCUGAAAC | mAGAUmGAGmGUUGmUmAmGmCUCCCmUm | 31355246 |
| | | GAUGAG (SEQ | CGUUGCUACAAUAAGGCCGUC | GmAmAmAmCmCGUUmGmCUAmCAAUAAGm | |
| | | ID NO: 388) | GAAAGAUGUGCCGCAACGCUC | GmCCmGmUmCmGmAmAmAmGmAmUGUGCm | |
| | | | UGCCUUCUGGCAUCGUU (SEQ | CGmCAAmCGCUCUmGmCCmUmUmCmUGGC | |
| | | | ID NO: 389) | AUCG*mU*mU (SEQ ID NO: 391) | |
| G032795 | HLA-B | CUCUGGGAA | CUCUGGGAAAGGAGGGGAAGA | mC*mU*mC*mUmGGGmAmAAmGGmAGGGG | chr6: 31355221- |
| | | AGGAGGGGA | UGAGUUGUAGCUCCCUGAAAC | mAAGAmUGAmGUUGmUmAmGmCUCCCmUm | 31355245 |
| | | AGAUGA (SEQ | CGUUGCUACAAUAAGGCCGUC | GmAmAmAmCmCGUUmGmCUAmCAAU*AAG | |
| | | ID NO: 392) | GAAAGAUGUGCCGCAACGCUC | mGmCCmGmUmCmGmAmAmAmGmAmUGUGC | |
| | | | UGCCUUCUGGCAUCGUU (SEQ | mCGmCAAmCGCUCUmGmCCmUmUmCmUGG | |
| | | | ID NO: 393) | CAUCG*mU*mU (SEQ ID NO: 394) | |
| G034209 | HLA-B | CUCUGGGAA | CUCUGGGAAAGGAGGGGAAGA | mC*mU*mC*mUmGGGmAmAAmGGmAGGGG | chr6: 31355221- |
| | | AGGAGGGGA | UGAGUUGUAGCUCCCUGAAAC | mAAGAmUGAmGUUGmUmAmGmCUCCCmUm | 31355245 |
| | | AGAUGA (SEQ | CGUUGCUACAAUAAGGCCGUC | GmAmAmAmCmCGUUmGmCUAmCAAUAAGm | |
| | | ID NO: 392) | GAAAGAUGUGCCGCAACGCUC | GmCCmGmUmCmGmAmAmAmGmAmUGUGCm | |
| | | | UGCCUUCUGGCAUCGUU (SEQ | CGmCAAmCGCUCUmGmCCmUmUmCmUGGC | |
| | | | ID NO: 393) | AUCG*mU*mU (SEQ ID NO: 395) | |
| G032806 | HLA-B | UCCCAGAGCC | UCCCAGAGCCGUCUUCCCAGU | mU*mC*mC*mCmAGAmGmCCmGUmCUUCCm | chr6: 31355205- |
| | | GUCUUCCCAG | CCAGUUGUAGCUCCCUGAAAC | CAGUmCCAmGUUGmUmAmGmCUCCCmUmG | 31355229 |
| | | UCCA (SEQ ID | CGUUGCUACAAUAAGGCCGUC | mAmAmAmCmCGUUmGmCUAmCAAU*AAGm | |
| | | NO: 396) | GAAAGAUGUGCCGCAACGCUC | GmCCmGmUmCmGmAmAmAmGmAmUGUGCm | |
| | | | UGCCUUCUGGCAUCGUU (SEQ | CGmCAAmCGCUCUmGmCCmUmUmCmUGGC | |
| | | | ID NO: 397) | AUCG*mU*mU (SEQ ID NO: 398) | |
| G034211 | HLA-B | UCCCAGAGCC | UCCCAGAGCCGUCUUCCCAGU | mU*mC*mC*mCmAGAmGmCCmGUmCUUCCm | chr6: 31355205- |
| | | GUCUUCCCAG | CCAGUUGUAGCUCCCUGAAAC | CAGUmCCAmGUUGmUmAmGmCUCCCmUmG | 31355229 |
| | | UCCA (SEQ ID | CGUUGCUACAAUAAGGCCGUC | mAmAmAmCmCGUUmGmCUAmCAAUAAGmG | |
| | | NO: 396) | GAAAGAUGUGCCGCAACGCUC | mCCmGmUmCmGmAmAmAmGmAmUGUGCmC | |
| | | | UGCCUUCUGGCAUCGUU (SEQ | GmCAAmCGCUCUmGmCCmUmUmCmUGGCA | |
| | | | ID NO: 397) | UCG*mU*mU (SEQ ID NO: 399) | |

1. A method of genetically modifying a cell, comprising:
(a) contacting the cell with a first genome editing tool, wherein the first genome editing tool comprises a first genomic editor and at least one guide RNA (gRNA) that targets at least one genomic locus and that is cognate to the first genomic editor; and
(b) contacting the cell with a second genome editing tool, wherein the second genome editing tool comprises a second genomic editor and at least one gRNA that targets at least one genomic locus and that is cognate to the second genomic editor, wherein the first genomic editor is orthogonal to the second genomic editor,
thereby producing at least two genome edits in the cell.

2. The method of claim 1, wherein the first genomic editor or the second genomic editor is delivered to the cell as at least one polypeptide or at least one polynucleotide that encodes the polypeptide.

3. (canceled)

4. (canceled)

5. The method of claim 1, wherein:
(a) the first genomic editor comprises a cleavase, a nickase, a catalytically inactive nuclease, a base editor, or a fusion protein comprising a DNA polymerase and a nickase; and/or
(b) the second genomic editor comprises a cleavase, a nickase, a catalytically inactive nuclease, a base editor, or a fusion protein comprising a DNA polymerase and a nickase.

6. (canceled)

7. The method of claim 1, wherein one of the first genomic editor and the second genomic editor comprises a base editor, and the other of the first genomic editor and the second genomic editor comprises a cleavase.

8. The method of claim 7, further comprising contacting the cell with a nucleic acid encoding an exogenous gene.

9. The method of claim 1, wherein one of the first genomic editor and second genomic editor comprises an N. meningitidis (Nme) RNA-guided nickase or cleavase, and the other of the first genomic editor and the second genomic editor comprises an S. pyogenes (Spy) RNA-guided nickase or cleavase.

10. (canceled)

11. The method of claim 1, wherein
(a) the first genomic editor comprises a base editor, and the at least one guide RNA (gRNA) that targets at least one genomic locus is cognate to the base editor; and
(b) the second genomic editor comprises an RNA-guided cleavase, and the at least one gRNA that targets at least one genomic locus is cognate to the RNA-guided cleavase, wherein the base editor is orthogonal to the RNA-guided cleavase.

12. The method of claim 11, further comprising:
culturing the cell, thereby producing a population of cells comprising edited cells comprising at least two genome edits per cell.

13. The method of claim 11, wherein the base editor is a C to T base editor, or is an A to G base editor.

14. The method of claim 1, wherein one of the at least two genome edits comprises a double-stranded break, and another one of the at least two genome edits comprises a transition or base edit.

15. (canceled)

16. The method of claim 1, wherein step (a) and step (b) are performed simultaneously.

17. (canceled)

18. The method of claim 1, wherein the first genomic editor is delivered to the cell as a nucleic acid comprising: (a) a nucleotide sequence that is at least 80%, 85%, 90%, 95%, 98%, or 100% identical to SEQ ID NO: 1, and the second genomic editor is delivered to the cell as a nucleic acid comprising a nucleotide sequence that is at least 80%, 85%, 90%, 95%, 98%, or 100% identical to any one of SEQ ID NOs: 180-190; or (b) a nucleotide sequence that is at least 80%, 85%, 90%, 95%, 98%, or 100% identical to SEQ ID NO: 147 or 310, and the second genomic editor is delivered to the cell as a nucleic acid comprising a nucleotide sequence that is at least 80%, 85%, 90%, 95%, 98%, or 100% identical to SEQ ID NO: 293 or 295.

19. (canceled)

20. (canceled)

21. (canceled)

22. (canceled)

23. The method of claim 1, wherein the first genomic editor comprises a Cas9 nickase.

24. (canceled)

25. (canceled)

26. The method of claim 1, wherein the second genomic editor comprises a Cas9 cleavase.

27. (canceled)

28. (canceled)

29. (canceled)

30. (canceled)

31. The method of claim 1, wherein: (a) at least one gRNA that is cognate to the first genomic editor is non-cognate to the second genomic editor; and/or (b) wherein at least one gRNA that is cognate to the second genomic editor is non-cognate to the first genomic editor.

32. (canceled)

33. The method of claim 1, wherein the at least one gRNA that is cognate to the first genomic editor comprises: (a) at least two gRNAs that target at least two different genomic loci, (b) at least three gRNAs that target at least three different genomic loci, (c) at least four gRNAs that target at least four different genomic loci, or (d) at least five gRNAs that target at least five different genomic loci.

34. (canceled)

35. (canceled)

36. (canceled)

37. A composition, comprising:
(a) a first genome editing tool, wherein the first genome editing tool comprises a first genomic editor, and at least one guide RNA (gRNA) that targets at least one genomic locus and that is cognate to the first genomic editor; and
(b) a second genome editing tool, wherein the second genome editing tool comprises a second genomic editor, and at least one gRNA that targets at least one genomic locus and that is cognate to the second genomic editor, wherein the first genomic editor is orthogonal to the second genomic editor.

38. The composition of claim 37, wherein the first genomic editor or the second genomic editor comprises at least one polypeptide or at least one mRNA; and/or
wherein the at least one gRNA comprises at least one polynucleotide that encodes the gRNA.

39. (canceled)

40. The composition of claim 37, wherein:
(a) the first genomic editor comprises a cleavase, a nickase, a catalytically inactive nuclease, a base editor, or a fusion protein comprising a DNA polymerase and a nickase; and/or
(b) the second genomic editor comprises a cleavase, a nickase, a catalytically inactive nuclease, a base editor, or a fusion protein comprising a DNA polymerase and a nickase.

41. (canceled)

42. The composition of claim 37, wherein one of the first genomic editor and the second genomic editor comprises a base editor, and the other of the first genomic editor and the second genomic editor comprises a cleavase.

43. The composition of claim 42, further comprising a nucleic acid encoding an exogenous gene.

44. The composition of claim 37, wherein one of the first genomic editor and the second genomic editor comprises a C to T base editor, and the other of the first genomic editor and the second genomic editor comprises an A to G base editor.

45. The composition of claim 37, wherein one of the first genomic editor and the second genomic editor comprises an N. meningitidis (Nme) RNA-guided nickase, and the other of the first genomic editor and the second genomic editor comprises an S. pyogenes (Spy) RNA-guided nickase.

46. (canceled)

47. A composition, comprising:
(a) a first genome editing tool, wherein the first genome editing tool comprises a first genomic editor comprising a base editor, and at least one guide RNA (gRNA) that targets at least one genomic locus and that is cognate to the base editor; and
(b) a second genome editing tool comprising a second genomic editor comprising an RNA-guided cleavase, and at least one gRNA that targets at least one genomic locus and that is cognate to the RNA-guided cleavase, wherein the base editor is orthogonal to the RNA-guided cleavase.

48. The composition of claim 47, wherein the base editor is a C to T base editor or is an A to G base editor.

49. (canceled)

50. (canceled)

51. The composition of claim 37, wherein the first genomic editor is a nucleic acid comprising: (a) a nucleotide sequence that is at least 80%, 85%, 90%, 95%, 98%, or 100% identical to SEQ ID NO: 1, and the second genomic editor is a nucleic acid comprising a nucleotide sequence that is at least 80%, 85%, 90%, 95%, 98%, or 100% identical to any one of SEQ ID NOs: 180-190; or (b) a nucleotide sequence that is at least 80%, 85%, 90%, 95%, 98%, or 100% identical to SEQ ID NO: 147 or 310, and the second genomic editor is a nucleic acid comprising a nucleotide sequence that is at least 80%, 85%, 90%, 95%, 98%, or 100% identical to SEQ ID NO: 293 or 295.

52. (canceled)

53. (canceled)

54. (canceled)

55. (canceled)

56. The composition of claim 37, wherein the first genomic editor comprises a Cas9 nickase.

57. (canceled)

58. (canceled)

59. The composition of claim 37, wherein the second genomic editor comprises a Cas9 cleavase.

60. (canceled)

61. (canceled)

62. (canceled)

63. (canceled)

64. The composition of claim 37, wherein: (a) the at least one gRNA that is cognate to the first genomic editor is non-cognate to the second genomic editor; and/or (b) the at least one gRNA that is cognate to the second genomic editor is non-cognate to the first genomic editor.

65. (canceled)

66. (canceled)

67. The composition of claim 37, wherein the at least one gRNA that is cognate to the first genomic editor comprises: (a) at least two gRNAs that target at least two different genomic loci, (b) at least three gRNAs that target at least three different genomic loci, (c) at least four gRNAs that target at least four different genomic loci, or (d) at least five gRNAs that target at least five different genomic loci.

68. (canceled)

69. (canceled)

70. (canceled)

71. The composition of claim 67, wherein the first genomic editor and one, two, three, four, five, or six of the at least one gRNA that are cognate to the first genomic editor and target different genomic loci are contained in a same lipid nanoparticle (LNP).

72. (canceled)

73. (canceled)

74. (canceled)

75. (canceled)

76. A cell, wherein the cell is treated in vitro with the method er of claim 1.

77. The cell of claim 76, wherein the cell is a human cell.

78. (canceled)

79. (canceled)

80. A population of cells, comprising the cell of claim 76.

81. (canceled)

82. A method for treating cancer, comprising administering to a subject in need thereof the cell of claim 76.

83. (canceled)

84. (canceled)
